(19) United States
(12) Patent Application Publication
(10) Pub. No.: US 2025/0296959 A1
(43) Pub. Date: Sep. 25, 2025

(54) B7-H3 TARGETING PEPTIDES AND CONSTRUCTS THEREOF

(71) Applicant: Mariana Oncology Inc., Watertown, MA (US)

(72) Inventors: Chunhui Huang, Arlington, MA (US); Chester A. Metcalf, III, Needham, MA (US); Punit Upadhyaya, Watertown, MA (US); Zhong Ma, Bedford, MA (US); Alonso Ricardo, Winchester, MA (US); Lihua Wu, Belmont, MA (US); Alok Singh, Sudbury, MA (US); Murray Wan, Medford, MA (US); Elisabetta Bianchi, Rome (IT); Willy Constantini, Rome (IT); Emanuela Nizi, Rome (IT)

(21) Appl. No.: 19/037,030

(22) Filed: Jan. 24, 2025

Related U.S. Application Data
(60) Provisional application No. 63/625,649, filed on Jan. 26, 2024.
(60) Provisional application No. 63/625,696, filed on Jan. 26, 2024.
(60) Provisional application No. 63/728,093, filed on Dec. 4, 2024.

(51) Int. Cl. C07K 7/64; A61K 38/00; A61K 51/08

(52) U.S. Cl. CPC C07K 7/64; A61K 51/088; A61K 38/00; A61K 2123/00

(57) ABSTRACT

The present disclosure relates to targeting moieties such as peptides that can bind to B7-H3. The disclosure also provides targeting constructs, which may include a targeting moiety attached, via an optional linker, to a chelating agent for association of a cargo. Methods of making the constructs and formulations thereof are also provided. Methods of using the constructs and/or formulations thereof to treat subjects, for example, to treat or prevent cancer, are also described.

### RELATED APPLICATIONS

This application claims priority to U.S. Provisional Application No. 63/625,649 filed on Jan. 26, 2024, U.S. Provisional Application No. 63/625,696 filed on Jan. 26, 2024, and U.S. Provisional Application No. 63/728,093 filed on Dec. 4, 2024. The entire contents of each application are hereby incorporated by reference in their entirety.

### SEQUENCE LISTING

This application contains a computer readable Sequence Listing that has been submitted in XML file format with the application, the content of which is incorporated by reference in its entirety. The Sequence Listing XML file submitted with this application is entitled “PAT059768-US-NP-SEQLIST,” was created on Jun. 9, 2025, and is 1,844,953 bytes in size.

### BACKGROUND

B7-H3, also known as CD276, is a transmembrane protein composed of either one or two pair(s) of IgV-like and IgC-like extracellular immunoglobulin domains, a transmembrane region, and a cytoplasmic tail. B7-H3 is an immune checkpoint molecule that produces a coinhibitory signal that decreases the activity of the Major Histocompatibility Complex and T cell receptor (MHC-TCR) signal between an antigen presenting cell (APC) and a T cell. By reducing the activity of the MHC-TCR signal, B7-H3 attenuates immune responses, thus preventing T cell proliferation and cytokine production while promoting interleukin 10 (IL-10) and transforming growth factor beta-1 (TGF-s1) production.

B7-H3 can be found in several different cellular compartments, but its expression is generally low in healthy cells. However, B7-H3 is highly expressed in tumor cell types, especially metastatic tumor cells, as well as activated immune cells in the tumor microenvironment. B7-H3 promotes the survival and progression of cancer cells by suppressing the immune system and promoting tumorigenic functions including epithelial-to-mesenchymal transition, migration, invasion, angiogenesis, and chemoresistance. Such behaviors have been documented in many types of cancer, including cervical cancer, gastric cancer, pancreatic carcinoma, and colorectal cancer (Li, et al., Oncol. Rep. 2017, 38(2):1043-1050; Li, et al., Oncotarget, 2017, 8(42): 71725-71735; Xie, et al., Sci Rep. 2016, 6:27528; Wang, et al., Cell Death Dis. 2020). As the expression and role of B7-H3 in cancer progression is ubiquitous, it has led to the correlation between B7-H3 expression and poor overall survival in cancer patients.

As B7-H3 plays a role in cancer progression and is specifically upregulated in tumor cells, it is a promising target for cancer treatment. Several therapeutics directed at B7-H3, including small molecule inhibitors and the monoclonal antibody Enoblituzumab, have reached early clinical trials. However, there remains a need for an effective cancer treatment that targets B7-H3.

### SUMMARY

The present disclosure relates to targeting moieties such as peptides, proteins and antibodies that can bind to B7-H3. The disclosure also provides targeting constructs, which may include a targeting moiety attached, via an optional linker, to a chelating agent for association of a cargo. In a particular aspect, the targeting construct comprises a targeting moiety that is a cyclic peptide that targets B7-H3, which is attached, via an optional linker, to a chelating agent for association of a radionuclide.

Accordingly, provided herein are cyclic peptides that target B7-H3. These peptides are useful in the treatment of a variety of indications, including cancer.

In an aspect, provided herein is a cyclic peptide comprising the amino acid sequence of Formula A:

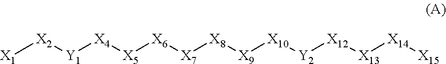

or a pharmaceutically acceptable salt and/or solvate thereof, wherein the cyclic peptide is cyclized via a linker between Y₁ and Y₂, wherein the remaining variables are defined herein, wherein a chelator is optionally linked to the cyclic peptide via a linking group.

In another aspect, provided herein is a cyclic peptide of Formula I:

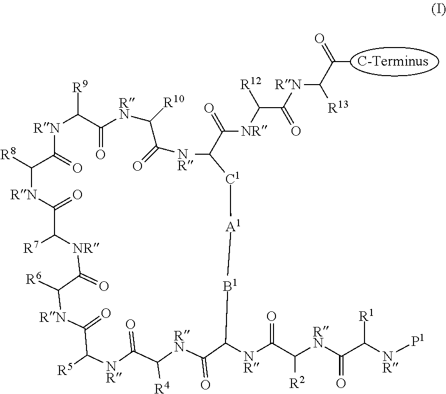

or a pharmaceutically acceptable salt thereof, where the variables are defined herein.

In some embodiments, the cyclic peptides provided herein are cyclic peptides of Formula I, or a pharmaceutically acceptable salt and/or solvate thereof.

In yet another aspect, provided herein is a cyclic peptide comprising the amino acid sequence of Formula B:

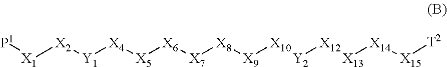

or a pharmaceutically acceptable salt and/or solvate thereof, wherein the cyclic peptide is cyclized via a linker between Y₁ and Y₂, wherein the remaining variables are defined herein, wherein a chelator is optionally linked to the cyclic peptide via a linking group. The chelator can be labeled with a radionuclide.

In still another aspect, provided herein is a cyclic peptide of Formula VI:

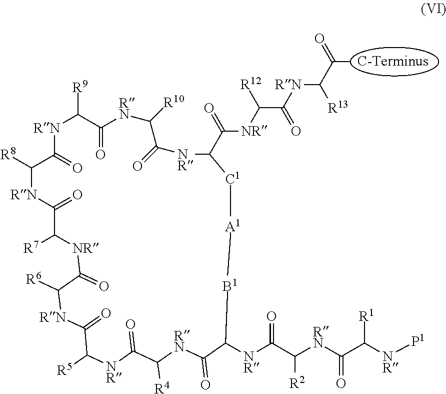

or a pharmaceutically acceptable salt thereof, wherein the variables are defined herein.

In some embodiments, the cyclic peptides provided herein are cyclic peptides of Formula VI, or a pharmaceutically acceptable salt and/or solvate thereof.

In an embodiment, the cyclic peptide of Formula I or Formula VI is attached, via an optional linker, to a chelating agent for labeling with a radionuclide.

In yet another embodiment, the cyclic peptide of Formula I or Formula VI is selected from a cyclic peptide in Table 1, or a pharmaceutically acceptable salt and/or solvate thereof. In yet another embodiment, the cyclic peptide of Formula I or Formula VI is selected from a cyclic peptide in Table 2, or a pharmaceutically acceptable salt and/or solvate thereof.

In still another embodiment, the chelating agent is selected from a chelating agent in Table 3. In some embodiments, the chelating agent labeled with a radionuclide. In some embodiments, the radionuclide is selected from the group consisting of ¹¹¹In, ^{99m}Tc, ^{94m}Tc, ⁶⁶Ga, ⁶⁷Ga, ⁶⁸Ga, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh, ¹⁶⁵Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹²³I, ¹²⁴I, ¹²⁵I, ¹³¹I, ⁷⁵Br, ⁷⁶Br, ⁷⁷Br, ⁸⁰Br, ⁸²Br, ¹⁸F, ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac. In some embodiments, the radionuclide is ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, or ²²⁷Th.

In an embodiment, the radionuclide is selected from a radionuclide in Table 4.

In another embodiment, the cyclic peptide, or a pharmaceutically acceptable salt or solvate thereof, is radiolabeled with F-18, Ga-68, In-111, Lu-177, or Ac-225. In a particular embodiment, the cyclic peptide, or a pharmaceutically acceptable salt or solvate thereof, is radiolabeled with Lu-177. In a particular embodiment, the cyclic peptide, or a pharmaceutically acceptable salt or solvate thereof, is radiolabeled with Ac-225.

In another aspect, provided herein is a pharmaceutical composition comprising a cyclic peptide described herein, or a pharmaceutically acceptable salt thereof, and a pharmaceutically acceptable carrier.

In yet another aspect, provided herein is a method of targeting B7-H3 in a subject in need thereof comprising administering to the subject a therapeutically effective amount of a compound described herein, or a pharmaceutically acceptable salt or solvate thereof.

In an aspect, provided herein is a method of imaging a subject, comprising administering to the subject a cyclic peptide of the present disclosure, or a pharmaceutical composition thereof, and obtaining an image of the subject

In still another aspect, provided herein is a method of treating cancer in a subject in need thereof comprising administering to the subject a therapeutically effective amount of a compound described herein. The cancer may include at least one cell comprising B7-H3. The cancer may be urothelial cancer, melanoma or squamous cell carcinoma. In other embodiments, the cancer is urothelial cancer, melanoma, lung cancer, squamous cell carcinoma, breast cancer, esophageal cancer, prostate cancer, liver cancer, endometrial cancer, sarcoma, bladder cancer, salivary gland cancer, renal cell carcinoma, gastric cancer, or pancreatic cancer. In an embodiment, the cancer is non-small cell lung cancer (NSCLC), small cell lung cancer (SCLC), triple-negative breast cancer (TNBC), Luminal A breast cancer, Luminal B breast cancer, HER2+ breast cancer, head and neck squamous cell carcinoma (HNSCC), or osteosarcoma.

In an aspect, provided herein is a peptide having binding specificity for B7-H3 isoform 4lg (4lg-B7-H3). In an embodiment, the peptide binds to one or more amino acids of D154, Q286, K291, M147, S234, T236, T238, and T290 of a 4lg-B7-H3 amino acid sequence of SEQ ID NO: 553. In another embodiment, the peptide binds to amino acids D154, Q286, K291, M147, S234, T236, T238, and T290 of a 4lg-B7-H3 amino acid sequence of SEQ ID NO: 553. In yet another embodiment, the peptide is cyclic. In still another embodiment, the peptide is non-cyclic. The chelating agent may include a polyaminocarboxylate agent. The chelating agent may include ethylenediamine tetraacetic acid (EDTA), diethylenetriamine pentaacetic acid (DTPA), 1,4,7,10-tetra-azacylcododecane-N,N′,N″,N″-tetraacetic acid (DOTA), 6-((16-((6-Carboxypyridin-2-yl)methyl)-1,4,10,13-tetraoxa-7,16-diazacyclooctadecan-7-yl)methyl)-4-isothiocyanatopicolinic acid (Macropa), Macrodipa, 2,2′,2″,2′-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, α-(2-carboxyethyl) (DOTAGA), 1,4,7-Triazacyclononane-N,N′,N″-triacetic acid (NOTA), 1,4,7,10-tetraazacyclododecane-N,N′,N″,N″-tetraacetic acid (TETA), 1,4,7,10,13-pentaazacyclopentadecane-N,N′,N″,N″,N″-pentaacetic acid (PEPA), and 1,4,7,10,13,16-hexaazacyclohexadecane-N,N′,N″,N″,N″,N″-hexaacetic acid (HEHA). In another embodiment, the chelator is 5,11,16,22-Tetraazahexacosanediamide (DFO*) or N,N′-1,4-Butanediylbis[N-[3-[[(1,6-dihydro-1-hydroxy-6-oxo-2-pyridinyl)carbonyl]amino]propyl]-1,6-dihydro-1-hydroxy-6-oxo-2-pyridinecarboxamide] (HOPO), or a derivative thereof.

The cargo may include a radioactive agent. The radioactive agent may include a radionuclide. Accordingly, in some embodiments, the constructs or compounds disclosed herein optionally comprise a radionuclide. In some embodiments, the constructs or compounds disclosed herein comprise a radionuclide. The radionuclide may be any of those listed in Table 4. In some embodiments, the radionuclide is selected from the group consisting of ¹¹¹In, ^{99m}Tc, ^{94m}Tc, ⁶⁶Ga, ⁶⁷Ga, ⁶⁸Ga, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh, ¹⁶⁵Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹²³I, ¹²⁴I, ¹²⁵I, ¹³¹I, ⁷⁵Br, ⁷⁶Br, ⁷⁷Br, ⁸⁰Br, ⁸²Br, ¹⁸F, ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac. In some embodiments, the radionuclide is ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, or ²²⁷Th. The optional linker may include a cleavable linker. The optional linker may include a non-cleavable linker. The optional linker may comprise at least one amino acid.

In some embodiments, the present disclosure provides a pharmaceutical composition including a construct and a pharmaceutically acceptable excipient.

In some embodiments, the present disclosure provides a method of delivering a cargo to a cell that includes contacting the cell or a subject comprising the cell with a construct or the pharmaceutical composition thereof. In an embodiment, the cargo can be a radioactive agent, such as a radionuclide. In other embodiments, the cargo is a cytotoxic agent.

In some embodiments, the present disclosure provides a method of treating a subject that includes administering a construct or the pharmaceutical composition thereof. The subject may have cancer. The cancer may include at least one cell comprising B7-H3. The cancer may be urothelial cancer, melanoma or squamous cell carcinoma. In other embodiments, the cancer is urothelial cancer, melanoma, lung cancer, squamous cell carcinoma, breast cancer, esophageal cancer, prostate cancer, liver cancer, endometrial cancer, sarcoma, bladder cancer, salivary gland cancer, renal cell carcinoma, gastric cancer, or pancreatic cancer. In an embodiment, the cancer is non-small cell lung cancer (NSCLC), small cell lung cancer (SCLC), triple-negative breast cancer (TNBC), Luminal A breast cancer, Luminal B breast cancer, HER2+ breast cancer, head and neck squamous cell carcinoma (HNSCC), or osteosarcoma.

### BRIEF DESCRIPTION OF THE DRAWINGS

FIG. 1A and FIG. 1B show a comparison of dose-dependent anti-cancer activity in CDX mouse models with high and regular specific activity [177Lu]Lu-496. FIG. 1A shows data for a SHP77 cell model. FIG. 1B shows data for a NCI-H1650 model.

FIG. 2 is an RP-HPLC chromatogram of small-scale HSA [²²⁵Ac]Ac-496 (Run #1).

FIG. 3 is an RP-HPLC chromatogram of small-scale HSA [²²⁵Ac]Ac-496 (Run #2).

FIG. 4A and FIG. 4B show tumor volume over time after a single IV administration of vehicle, [²²⁵Ac]Ac-496 in Study 1 (A), and Study 2 (B) in NCI-H1650 Tumor-Bearing Mice (MEAN±SEM).

### DETAILED DESCRIPTION

Provided here are compounds that target B7-H3. In particular, provided herein are targeting constructs (also referred to herein as “compounds”) comprising a targeting moiety that is a cyclic peptide that targets B7-H3, which is attached, via an optional linker, to a chelating agent for association of a radionuclide. As such, these compounds, as well as pharmaceutical compositions that comprise these compounds, are useful in the treatment of a variety of indications, including cancer. In an embodiment, the cyclic peptide can comprise one or more chelators, wherein the chelator may be associated with a radionuclide.

### I. Compounds and Compositions

In some embodiments, the present disclosure relates to targeting moieties such as peptides, proteins and antibodies that can bind to targets. In some embodiments, the present disclosure provides constructs capable of localizing to and/or associating with targets. Such constructs that include any combination of a targeting moiety and a cargo are referred to herein as “targeting constructs.” As used herein, the term “targeting moiety” refers to a component of a targeting construct or combination of components involved in targeting construct localization to or association with a target. Cargo components of targeting constructs may include any one of a variety of compounds, including, but not limited to, chemical compounds, biomolecules, metals, polymeric molecules, therapeutic agents, cytotoxic agents, and radioactive agents. The chelator can be associated with a payload such as, e.g., a radionuclide or cytotoxic agent.

In a particular embodiment, the targeting construct comprises a targeting moiety that is a cyclic peptide that targets B7-H3, which is attached, via an optional linker, to a chelating agent for association of a radionuclide.

### Targets

Targeting constructs may be directed to a variety of targets. In some embodiments, targeting constructs may target cells. In an embodiment, the cargo can be a radioactive agent, such as a radionuclide. Such targeting constructs may include targeting moieties that may target cell antigens, including those associated with target cell surfaces. In this case, the cell antigen is the target of the targeting moieties and the targeting constructs. An “antigen,” as referred to herein, is any entity that binds to a specific antibody or T-cell receptor in an organic and therefore is capable of inducing an immune response in an organism. Immune responses are reactions of cells, tissues and/or organs of an organism to an antigen, such as a foreign entity. Immune responses typically lead to the production of one or more antibodies against the foreign entity by an organism. As used herein, the term “target antigen” refers to a molecule, peptide, protein, or epitope to which an antibody binds or for which an antibody is desired, designed, or developed to have affinity for. Such target antigens may include cancer cell antigens, for example, those expressed on cancer cell surfaces.

In some embodiments, target antigens of the present disclosure include B7-H3 or portions thereof. B7-H3 antigens may include B7-H3 extracellular domains. B7-H3 antigens may include fusion proteins of B7-H3 or other entities comprising B7-H3 portions.

### Targeting Moieties

In some embodiments, targeting moieties localize targeting constructs to targets by binding such targets or associated components. Targeting moieties may bind to cells or biomolecules or other structures associated with cells. For example, in some embodiments, targeting moieties bind to cell antigens. Such cell antigens may be specifically expressed by, expressed on, or otherwise associated with specific cell types. Specific cell types may be characterized by one or more of cell size, age, shape, location, tissue of origin, organ of origin, function, activity, genotype, phenotype, or association with disfunction or disease. Targeting moieties may bind to cancer cell antigens. In some embodiments, targeting moieties bind to B7-H3. In some embodiments, targeting moieties bind to human B7-H3.

Targeting moieties may include or consist of proteins, peptides, antibodies, nucleic acids, nucleic acid analogs, aptamers, lipids, carbohydrates, glycoproteins, or small molecules. In some embodiments, the targeting moieties include or consist of peptides, antibodies or fragments or variants thereof. In a particular embodiment, the targeting moiety is a cyclic peptide.

In some embodiments, targeting moieties of the disclosure, such as peptides and antibodies, have an affinity for human B7-H3. In some embodiments, targeting moieties of the disclosure have an affinity for human B7-H3 within identified ranges as measured in conventional assays. “Affinity” or “binding affinity” means the strength of the sum total of noncovalent interactions between a single binding site of a molecule (e.g., an antibody or peptide binding compound) and its binding partner (e.g., an antigen). Unless indicated otherwise, as used herein, “binding affinity” refers to intrinsic binding affinity which reflects all interaction between members of a binding pair (e.g., antibody or peptide binding compound and antigen). The affinity of a molecule X for its partner Y can generally be represented by the dissociation constant (K_{D}). The equilibrium dissociation constant (K_{D}) is calculated as the ratio k_{off}/kₒₙ.

Low-affinity targeting moieties generally bind antigen slowly and tend to dissociate readily, whereas high-affinity targeting moieties generally bind antigen faster and tend to remain bound longer. A variety of methods of measuring binding affinity are known in the art, any of which can be used for purposes of the present disclosure.

In some embodiments, the targeting moieties disclosed herein may bind to a target protein with an equilibrium dissociation constant (K_{D}) of from about 0.001 nM to about 0.01 nM, from about 0.005 nM to about 0.05 nM, from about 0.01 nM to about 0.1 nM, from about 0.05 nM to about 0.5 nM, from about 0.1 nM to about 1.0 nM, from about 0.5 nM to about 5.0 nM, from about 2 nM to about 10 nM, from about 8 nM to about 20 nM, from about 15 nM to about 45 nM, from about 30 nM to about 60 nM, from about 40 nM to about 80 nM, from about 50 nM to about 100 nM, from about 75 nM to about 150 nM, from about 100 nM to about 500 nM, from about 200 nM to about 800 nM, from about 400 nM to about 1,000 nM or at least 1,000 nM. In some embodiments, the target protein is B7-H3.

In some embodiments, the K_{D} is determined by Surface Plasmon Resonance (SPR). An exemplary SPR protocol is provided in Example 2.

### Peptides

In some embodiments, targeting moieties of the present disclosure are peptides. According to the present disclosure, any amino acid-based molecule (natural or unnatural) may be termed a “peptide” and this term embraces “peptides,” “peptidomimetics,” and “proteins.” “Peptides” are traditionally considered to range in size from about 4 to about 50 amino acids. Peptides larger than about 50 amino acids are generally termed “proteins.”

Peptides of the present disclosure may be cyclic. In particular, provided herein are cyclic peptides that target B7-H3. Cyclic peptides include any peptides that have as part of their structure one or more cyclic features such as a loop and/or an internal linkage. In some embodiments, cyclic peptides are formed when a molecule acts as a bridging moiety to link two or more regions of the peptide.

As used herein, the term “bridging moiety” refers to one or more components of a bridge formed between two adjacent or non-adjacent amino acids, unnatural amino acids or non-amino acids in a peptide. Bridging moieties may be of any size or composition. In some embodiments, bridging moieties may comprise one or more chemical bonds between two adjacent or non-adjacent amino acids, unnatural amino acids, non-amino acid residues or combinations thereof. In some embodiments, such chemical bonds may be between one or more functional groups on adjacent or non-adjacent amino acids, unnatural amino acids, non-amino acid residues or combinations thereof. Bridging moieties may include one or more of an amide bond (lactam), disulfide bond, thioether bond, aromatic ring, triazole ring, and hydrocarbon chain. In some embodiments, bridging moieties include an amide bond between an amine functionality and a carboxylate functionality, each present in an amino acid, unnatural amino acid or non-amino acid residue side chain. In some embodiments, the amine or carboxylate functionalities are part of a non-amino acid residue or unnatural amino acid residue.

In some embodiments, the present disclosure provides peptides that bind to human B7-H3. Such cells may include cancer cells, such as but not limited to lung cancer cells, breast cancer cells, bladder cancer cells, colon cancer cells, urothelial cancer cells, melanoma cells, esophageal cancer cells, head and neck cancer cells, or squamous cell carcinoma cells.

### Cyclic Peptides

In some embodiments, peptides of the present disclosure can comprise cyclic peptides having one or more bridging moieties (e.g., cyclic structure, staple, bridge, etc.). Peptide stapling/bridging is a macrocyclization approach in which peptides are covalently modified through the formation of a chemical linkage (e.g., staple, bridge moiety, etc.) between the side chains of two amino acids. More specifically, peptides are rendered macrocyclic by formation of covalent bonds between atoms present within the linear peptide and atoms of a bridging moiety. Stapling/bridging can be used to constrain peptides into preferred bioactive conformations (reducing conformational flexibility and degrees of rotational freedom), thereby improving affinity for specific receptor targets and improving overall pharmacokinetics. The residues being linked are generally located on the same face of the peptide helix and separated by one, two, or three helical turns (e.g., a first amino acid at position (z) is linked to a second amino acid at position z+4, z+7, or z+11). In some embodiments, bridging moieties may comprise one or more chemical bonds between two adjacent or non-adjacent amino acids, unnatural amino acids, non-amino acid residues or combinations thereof. In some embodiments, such chemical bonds may be between one or more functional groups on adjacent or non-adjacent amino acids, unnatural amino acids, non-amino acid residues or combinations thereof.

Accordingly, in an aspect, provided herein is a cyclic peptide comprising the amino acid sequence of Formula A:

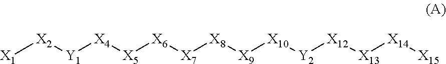

or a pharmaceutically acceptable salt and/or solvate thereof,
wherein:
X₁, X₂, X₅, X₆, X₇, X₉, X₁₀, X₁₂, and X₁₃ are each independently a natural or unnatural amino acid;
Y₁ is Cys or NMeCys;
X₄ is Val or Tbg;
X₈ is Asp or CysAcid;
Y₂ is Cys or NMeCys;
X₁₄ and X₁₅ are each independently any natural or unnatural amino acid or absent;
wherein one of X₅, X₆, X₇, X₉, or X₁₀ is substituted with a Chelator, wherein the Chelator is optionally linked to the cyclic peptide via a linking group; and
wherein the cyclic peptide is cyclized via a linker between Y₁ and Y₂.

In an embodiment, the cyclic peptide binds to B7-H3.

In an embodiment, one of X₅, X₆, X₇, X₉, or X₁₀ is substituted with a Chelator and an optional linking group, wherein the amino acid side chain of X₅, X₆, X₇, X₉, or X₁₀ is selected from L³-L³-Chelator,

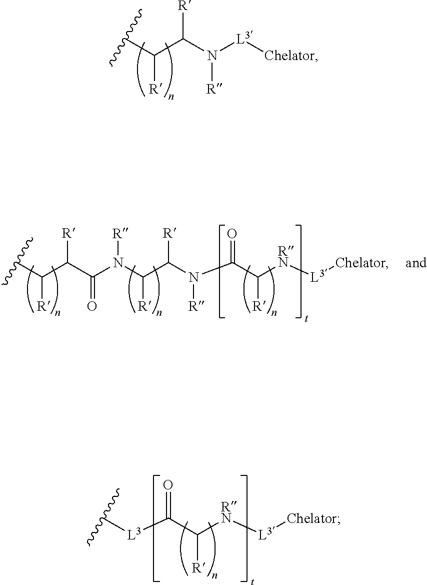

wherein:
L³ is absent or selected from

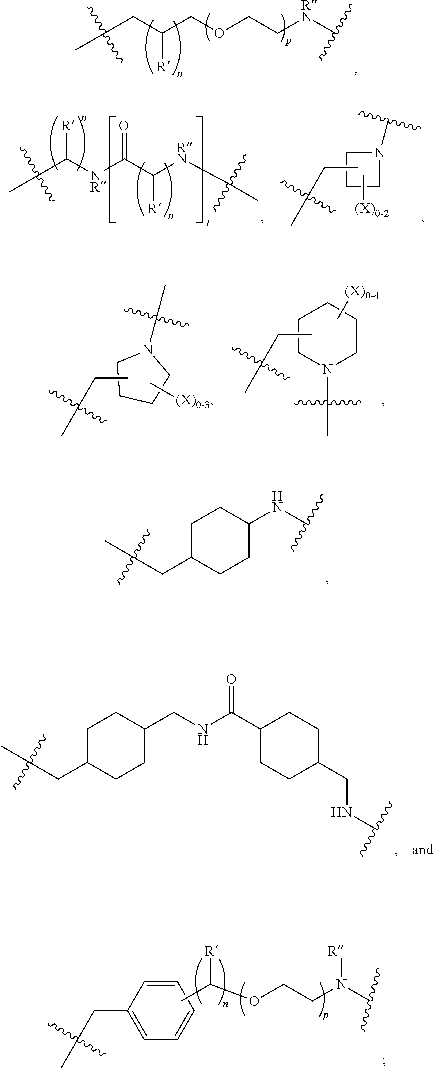

L^{3′} is absent or selected from

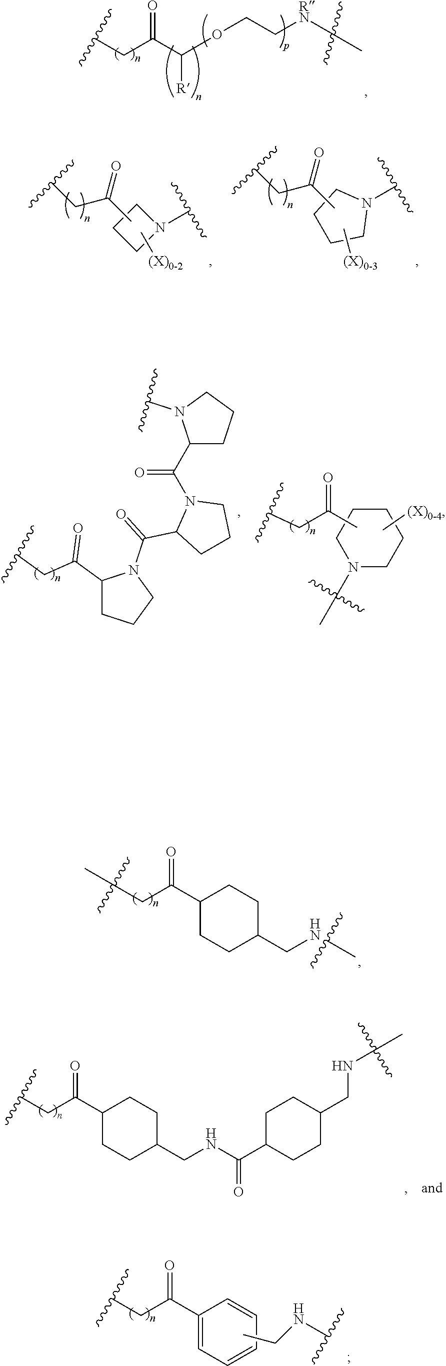

each n is independently an integer from 0 to 16;
each p is independently an integer from 0 to 30;
each t is independently 0, 1, 2, 3, 4, 5, or 6;
X is independently, for each occurrence, selected from halo, OH, C₁-C₆ alkyl, C₁-C₆ haloalkyl, N(R″)₂, and —O—(C₁-C₆ alkyl);
R′ is independently, for each occurrence, selected from H, OH, C₁-C₆ alkyl, C₁-C₆ haloalkyl, C₁-C₈ alkylamine, C₁-C₈ alkyl-OH, C(O)H, C(O)(C₁-C₆ alkyl), C(O)OH, C(O)O(C₁-C₆ alkyl), (C₁-C₆ alkyl)C(O)OH, (C₁-C₆ alkyl)C(O)O(C₁-C₆ alkyl), (C₁-C₆ alkyl)CO(C₁-C₆ alkyl), (C₁-C₆ alkyl)CS(C₁-C₆ alkyl), C(O)N(R″)₂, N(R″)₂, and N(R″)₃⁺; and
R″ is independently, for each occurrence, selected from H, C₁-C₆ alkyl, C₁-C₆ haloalkyl, (C₁-C₆ alkyl)C(O)OH, (C₁-C₆ alkyl)C(O)N(alkyl)₂, and C(O)O(C₁-C₆ alkyl).

In another embodiment, at least one of X₆ or X₇ is substituted with a Chelator and an optional linking group.

In yet another embodiment, X₁ is hLeu or hCha; and X₂ is Tyr.

In still another embodiment, the N-terminus and/or C-terminus of the cyclic peptide is capped. In still another embodiment, the N-terminus of the cyclic peptide is capped with P¹, wherein P¹ is selected from R″, —SO₂R″,

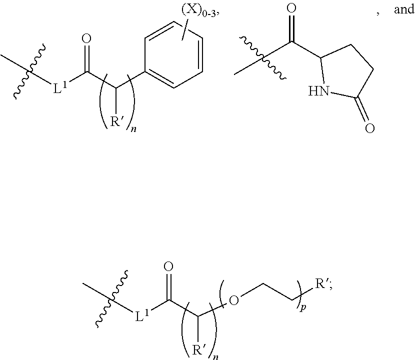

L¹ is absent or selected from:

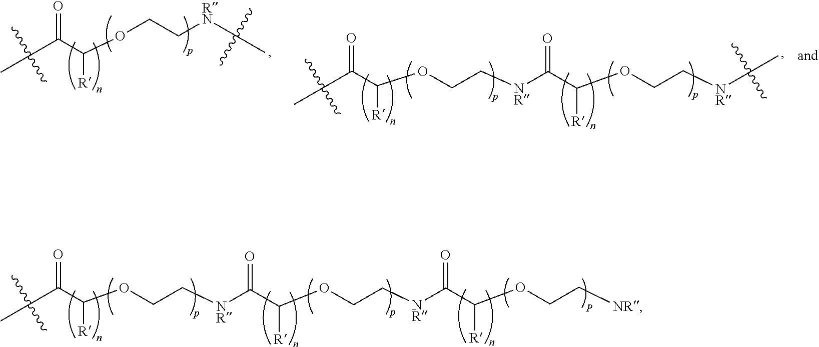

wherein the amino group of L¹ connects to the carbonyl group of P¹ to form an amide bond;
X is independently, for each occurrence, selected from halo, OH, C₁-C₆ alkyl, C₁-C₆ haloalkyl, N(R″)₂, and —O—(C₁-C₆ alkyl);
each n is independently an integer from 0 to 16;
each p is independently an integer from 0 to 30;
R′ is independently, for each occurrence, selected from H, OH, C₁-C₆ alkyl, C₁-C₆ haloalkyl, C₁-C₈ alkylamine, C₁-C₈ alkyl-OH, C(O)H, C(O)(C₁-C₆ alkyl), C(O)OH, C(O)O(C₁-C₆ alkyl), (C₁-C₆ alkyl)C(O)OH, (C₁-C₆ alkyl)C(O)O(C₁-C₆ alkyl), (C₁-C₆ alkyl)CO(C₁-C₆ alkyl), (C₁-C₆ alkyl)CS(C₁-C₆ alkyl), C(O)N(R″)₂, N(R″)₂, and N(R″)₃⁺; and
R″ is independently, for each occurrence, selected from H, C₁-C₆ alkyl, C₁-C₆ haloalkyl, (C₁-C₆ alkyl)C(O)OH, (C₁-C₆ alkyl)C(O)N(alkyl)₂, and C(O)O(C₁-C₆ alkyl).

In an embodiment, the C-terminus of the peptide is capped with R or N(R″)P²; P² is selected from R″,

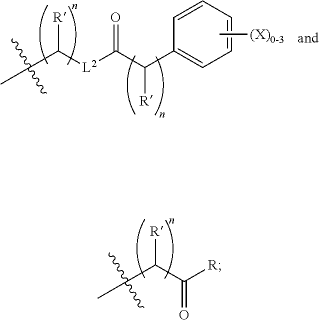

L² is absent or is selected from:

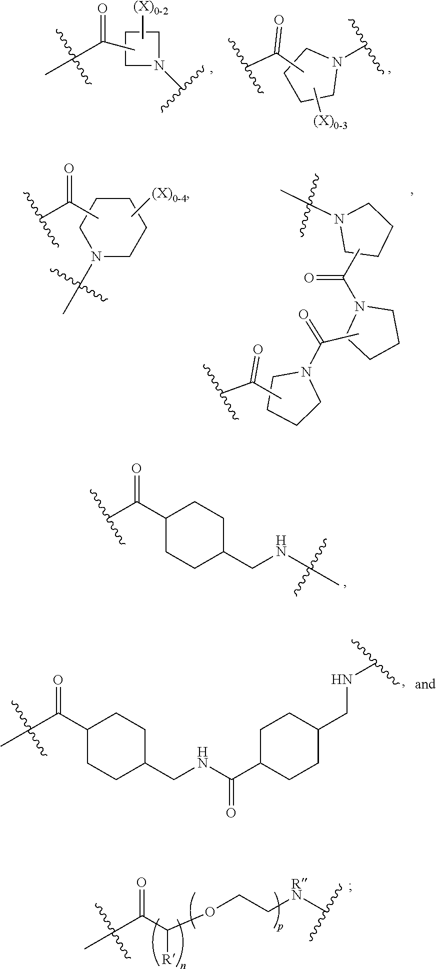

wherein the amino group of L² connects to the carbonyl group of P² to form an amide bond;
each n is independently an integer from 0 to 16;
p is an integer from 0 to 30;
X is independently, for each occurrence, selected from halo, OH, C₁-C₆ alkyl, C₁-C₆ haloalkyl, N(R″)₂, and —O—(C₁-C₆ alkyl);
R is selected from H, C₁-C₂₀ alkyl, C₁-C₆ haloalkyl, OH, —O—(C₁-C₆ alkyl), (C₁-C₆ alkyl)-OH, and N(R″)₂;
R′ is independently, for each occurrence, selected from H, OH, C₁-C₆ alkyl, C₁-C₆ haloalkyl, C₁-C₈ alkylamine, C₁-C₈ alkyl-OH, C(O)H, C(O)(C₁-C₆ alkyl), C(O)OH, C(O)O(C₁-C₆ alkyl), (C₁-C₆ alkyl)C(O)OH, (C₁-C₆ alkyl)C(O)O(C₁-C₆ alkyl), (C₁-C₆ alkyl)CO(C₁-C₆ alkyl), (C₁-C₆ alkyl)CS(C₁-C₆ alkyl), C(O)N(R″)₂, N(R″)₂, and N(R″)₃⁺; and
R″ is independently, for each occurrence, selected from H, C₁-C₆ alkyl, C₁-C₆ haloalkyl, (C₁-C₆ alkyl)C(O)OH, (C₁-C₆ alkyl)C(O)N(alkyl)₂, and C(O)O(C₁-C₆ alkyl).

In another embodiment, X₁ is selected from Ala, Cha, dAla, hCba, hCha, hLeu, HomoArg, HomoGlu, hTba, Leu, Met, and Nle. In yet another embodiment, X₁ is selected from hLeu, hCha, hCba and Nle, In still another embodiment, X₁ is hLeu or hCha. In an embodiment, X₁ is hLeu. In an embodiment, X₁ is hCha. In an embodiment, X₁ is substituted with an N-terminus group selected from P¹.

In another embodiment, X₂ is selected from 3CIY, 3FY, 4COOHPhe, 4FPhe, 4PyA, 7AzaW, Ala, alloThr, aMeNle, aMeTyr, Cba, Chg, dAla, FSY, HomoArg, HomoGlu, hTyr, hyVal, Nle, NMeNle, OMeTyr, Tba, ThpA, ThpG, Thr, and Tyr. In yet another embodiment, X₂ is selected from 4COOHPhe, 4FPhe, aMeTyr, hTyr, OMeTyr, ThpA, and Tyr. In still another embodiment, X₂ is Tyr or 4FPhe. In an embodiment, X₂ is Tyr.

In another embodiment, the linker between Y₁ and Y₂ is selected from a bond, C₁-6 alkylene, and

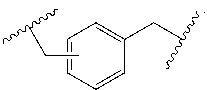

In yet another embodiment, Y₁ is Cys. In still another embodiment, Y₁ is NMeCys.

In another embodiment, Y₂ is Cys. In still another embodiment, Y₂ is NMeCys.

In an embodiment, the linker between Y₁ and Y₂ is a bond.

In an embodiment, X₄ is Tbg. In another embodiment, X₄ is Val.

In yet another embodiment, X₅ is selected from Ala, dAla, Gly, HomoGlu, N(CH₂)₂COOHGly, NetGly, Ser, and Sar. In still another embodiment, X₅ is Gly or Sar. In an embodiment, X₅ is Gly. In another embodiment, X₅ is Sar.

In some embodiments, X₅ can be substituted with a chelator, wherein the chelator is optionally linked to the cyclic peptide via a linking group. In an embodiment, X₅ is Lys(DOTA) or dLys(DOTA). In another embodiment, X₅ is Lys(DOTA). In yet another embodiment, X₅ is dLys(DOTA).

In still another embodiment, X₆ is selected from Ala, aMeLys, Arg, Aze, Cit, Dab, dAla, Dap, dPro, HomoGlu, hSer, Lys, Lys(Ac), Lys(Me)₂, Lys(Me)₃, Nle, NMeLys, Orn, Pip, Pip(Ac)Ala, Pip(C₄diacid)Ala, Pip(CH₂COOH)Ala, Pip(CONH₂)Ala, Pip(Me)Ala, PipAla, Pro, ThpA, and ₄PyA. In an embodiment, X₆ is selected from Lys, PipAla, ThpA, Cit, Pip(CH₂COOH)Ala, Lys(Ac), Pip(Ac)Ala, Pip(CONH₂)Ala, Pip(Me)Ala, Lys(Me)₂, and Lys(Me)₃. In another embodiment, X₆ is selected from Lys, Pip(CH₂COOH)Ala, and PipAla. In yet another embodiment, X₆ is Pip(CH₂COOH)Ala or Lys. In still another embodiment, X₆ is Pip(CH₂COOH)Ala. In some embodiments, X₆ can be substituted with a chelator, wherein the chelator is optionally linked to the cyclic peptide via a linking group. In an embodiment, X₆ is selected from Lys(gE-DOTA), Lys(gE-gE-DOTA), Lys(PEG4-DOTA), Pip(DOTA)Ala, Pip(PEG4-DOTA)Ala, Pip(PEG2-DOTA)Ala, Pip(gE-DOTA)Ala, Pip(R-DOTAGA)Ala, Pip(gE-gE-DOTA)Ala, and Glu[εLys(OH)-Val-Met-AmBz-DOTA]. In another embodiment, X₆ is Lys(DOTA) or Pip(PEG4-DOTA)Ala. In yet another embodiment, X₆ is Lys(DOTA). In still another embodiment, X₆ is Pip(PEG4-DOTA)Ala.

In an embodiment, the cyclic peptide comprising the amino acid sequence of Formula A is a cyclic peptide comprising the amino acid sequence of Formula Ai:

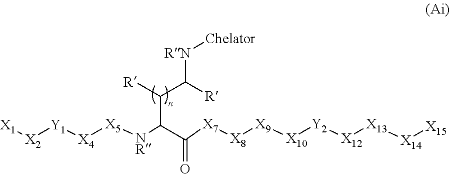

or a pharmaceutically acceptable salt and/or solvate thereof,
wherein:
R′ is independently, for each occurrence, selected from H, C(O)OH, (CH₂)OH, and NHAc;
R″ is independently, for each occurrence, H or CH₃; and
n is an integer from 0 to 16.

In another embodiment, the cyclic peptide comprising the amino acid sequence of Formula A is a cyclic peptide comprising the amino acid sequence of Formula Aii:

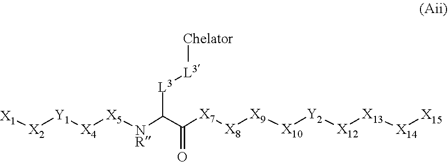

or a pharmaceutically acceptable salt and/or solvate thereof,
wherein:
L³ is absent or selected from

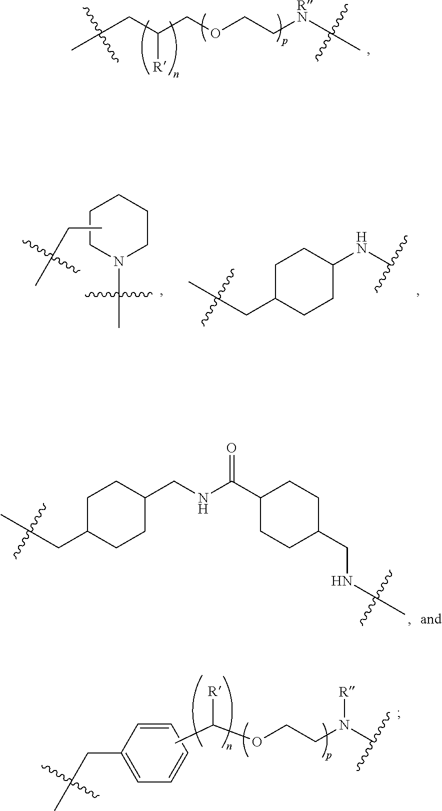

L^{3′} is absent or selected from

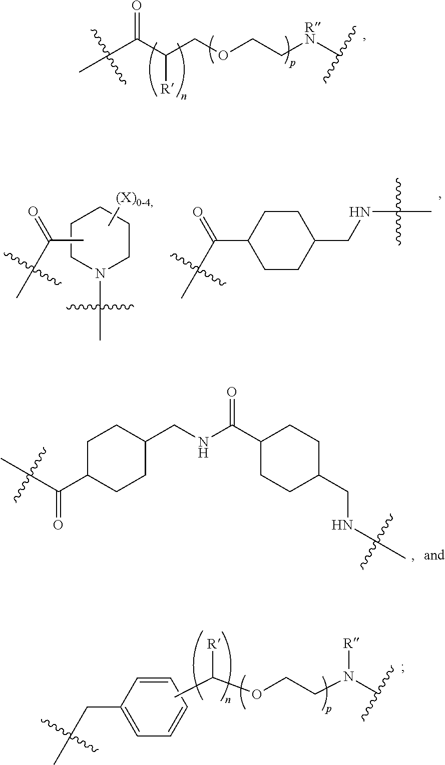

R′ is independently, for each occurrence, selected from H, C(O)OH, (CH₂)OH, and NHAc;
R″ is independently, for each occurrence, H or CH₃; and
each n is independently an integer from 0 to 16;
each p is independently an integer from 0 to 30.

In yet another embodiment, the cyclic peptide comprising the amino acid sequence of Formula A is a cyclic peptide comprising the amino acid sequence of Formula Aiii:

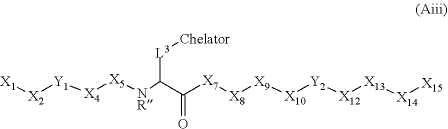

or a pharmaceutically acceptable salt and/or solvate thereof,
wherein:
L³ is selected from

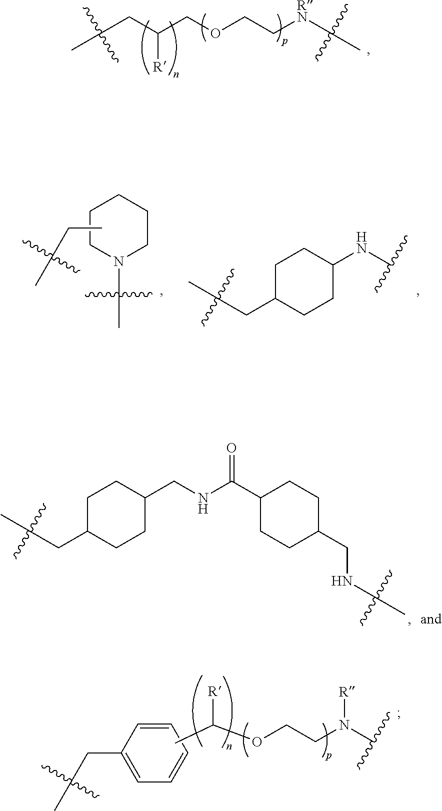

each n is independently an integer from 0 to 16;
each p is independently an integer from 0 to 30;
R′ is independently, for each occurrence, selected from H, C(O)OH, (CH₂)OH, and NHAc; and
R″ is independently, for each occurrence, H or CH₃.

In still another embodiment of the above formulae, X₇ is selected from (N-Me-ThpA), 4COOHPhe, 4OHCha, 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, and Tyr. In an embodiment, X₇ is selected from Tyr, CHA, PipAla, 4PyA, ThpA, 4COOHPhe, Pip(CH2COOH)Ala, Pip(DOTA)Ala, and 4OHCha. In another embodiment, X₇ is selected from 4PyA and ThpA. In yet another embodiment, X₇ is ThpA.

In some embodiments of the above formulae, X₇ can be substituted with a chelator, wherein the chelator is optionally linked to the cyclic peptide via a linking group. In an embodiment, X₇ is selected from AEF(DOTA), Lys(DOTA), MAF(DOTA), MAF(gE-DOTA), MAF(gE-gE-DOTA), MAF(PEG4-DOTA), MAF(gE-gE-gE-DOTA), MAF(PEG8-DOTA), Pip(DOTA)Ala, Pip(PEG4-DOTA)Ala, Pip(PEG2-DOTA)Ala, Pip(gE-DOTA)Ala, Pip(R-DOTAGA)Ala, Glu[εLys(OH)-Val-Met-AmBz-DOTA], Lys(PEG4-DPTA), and Pip(Glu-DOTA)Ala. In another embodiment, X₇ is Lys(DOTA). In another embodiment, X₇ is Pip(PEG4-DOTA)Ala.

In yet another embodiment, the cyclic peptide comprising the amino acid sequence of Formula A is a cyclic peptide comprising the amino acid sequence of Formula Aiv:

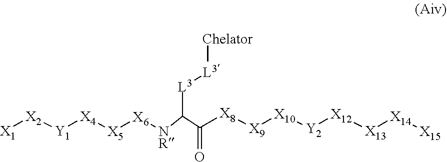

or a pharmaceutically acceptable salt and/or solvate thereof,
wherein:
L³ is absent or selected from

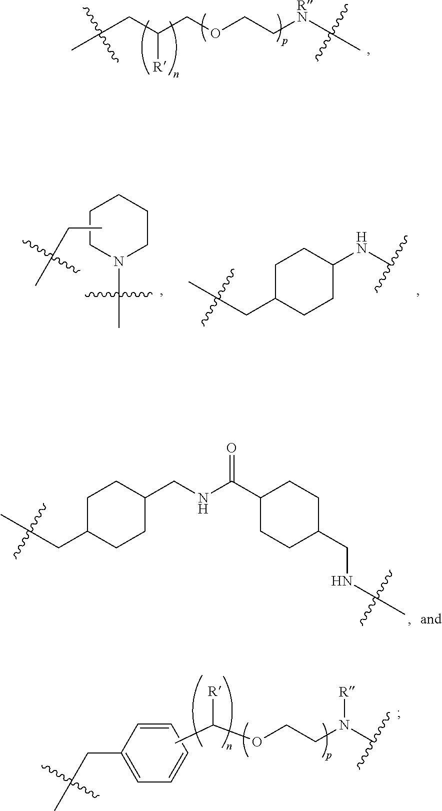

L^{3′} is absent or selected from

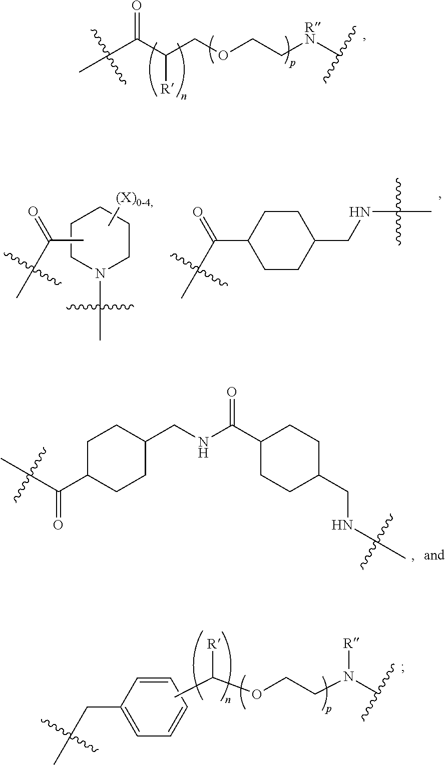

each n is independently an integer from 0 to 16;
each p is independently an integer from 0 to 30;
X is independently, for each occurrence, selected from halo, OH, C₁-C₆ alkyl, C₁-C₆ haloalkyl, N(R″)₂, and —O—(C₁-C₆ alkyl);
R′ is independently, for each occurrence, selected from H, C(O)OH, (CH₂)OH, and NHAc; and
R″ is independently, for each occurrence, H or CH₃.

In yet another embodiment, the cyclic peptide comprising the amino acid sequence of Formula A is a cyclic peptide comprising the amino acid sequence of Formula Av:

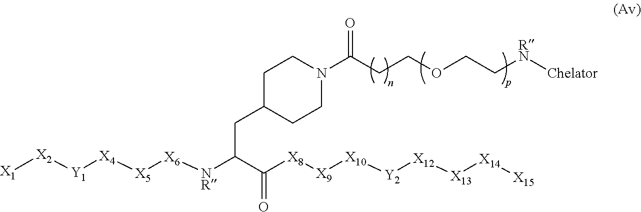

or a pharmaceutically acceptable salt and/or solvate thereof,
wherein:
n is an integer from 0 to 16;
p is an integer from 0 to 30; and
R″ is independently, for each occurrence, H or CH₃.

In an embodiment of the cyclic peptide of Formula Av, or a pharmaceutically acceptable salt thereof, n is 1. In yet another embodiment of the cyclic peptide of Formula Av or a pharmaceutically acceptable salt thereof, p is 4.

In still another embodiment, the cyclic peptide comprising the amino acid sequence of Formula A is a cyclic peptide comprising the amino acid sequence of Formula Avi:

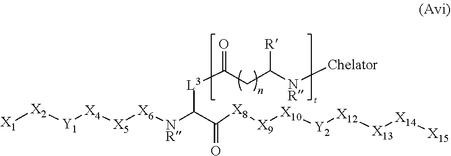

or a pharmaceutically acceptable salt and/or solvate thereof,
wherein:
L³ is absent or is selected from

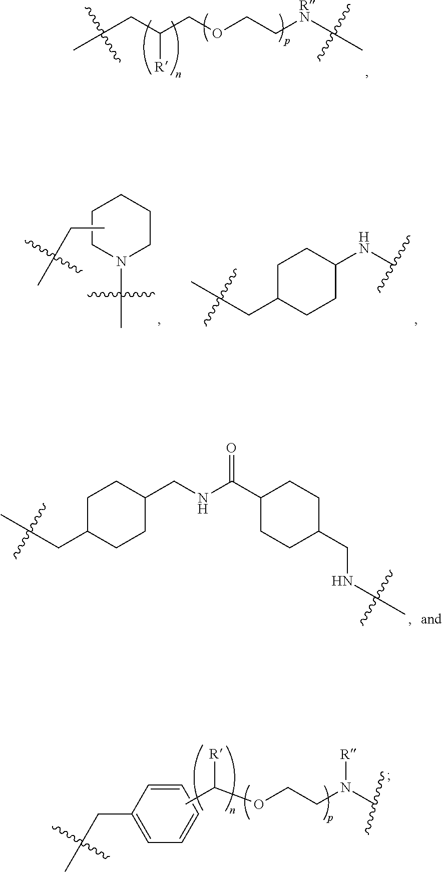

each n is independently an integer from 0 to 16;
p is an integer from 0 to 30;
t is 0, 1, 2, 3, 4, 5, or 6;
R′ is independently, for each occurrence, selected from H, C(O)OH, (CH₂)OH, and NHAc; and
R″ is independently, for each occurrence, H or CH₃.

In an embodiment, the cyclic peptide comprising the amino acid sequence of Formula A is a cyclic peptide comprising the amino acid sequence of Formula Avi:

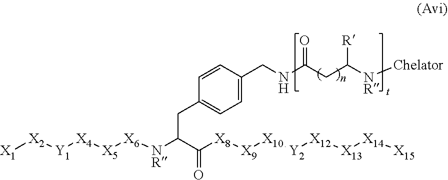

or a pharmaceutically acceptable salt and/or solvate thereof;
wherein:
n is an integer from 0 to 16;
t is 0, 1, 2, 3, 4, 5, or 6;
R′ is independently, for each occurrence, selected from H, C(O)OH, (CH₂)OH, and NHAc; and
R″ is independently, for each occurrence, H or CH₃.

In another embodiment, the cyclic peptide comprising the amino acid sequence of Formula A is a cyclic peptide comprising the amino acid sequence of Formula Avii:

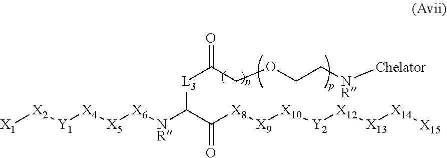

or a pharmaceutically acceptable salt and/or solvate thereof,
wherein:
L³ is absent or is selected from

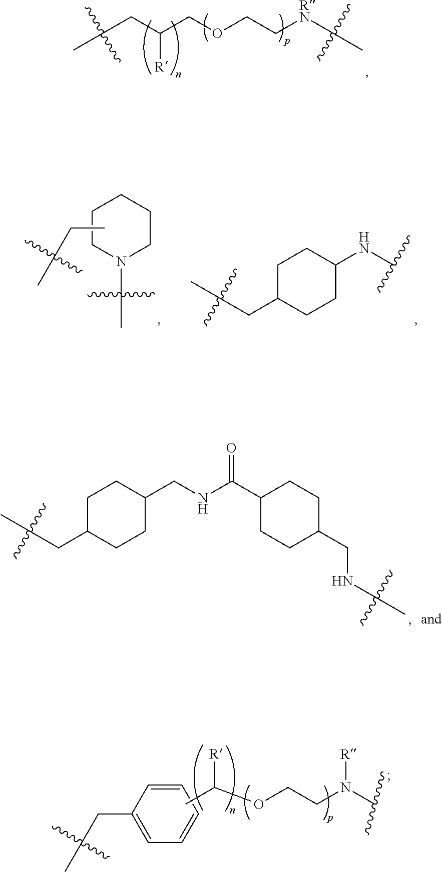

each n is independently an integer from 0 to 16;
each p is independently an integer from 0 to 30;
R′ is independently, for each occurrence, selected from H, C(O)OH, (CH₂)OH, and NHAc; and
R″ is independently, for each occurrence, H or CH₃.

In an embodiment of the above formulae, X₈ is Asp. In an embodiment, X₈ is CysAcid.

In another embodiment of the above formulae, X₉ is selected from Abu, Aib, Ala, allolle, alloThr, aMeSer, aMeVal, ChG, Cpg, dAla, HomoGlu, hSer, hydroVal, Nle, NMeVal, Nva, Ser, Tbg, Thr, and Val. In yet another embodiment, X₉ is Val.

In some embodiments of the above formulae, X, can be substituted with a chelator, wherein the chelator is optionally linked to the cyclic peptide via a linking group. In an embodiment, X₉ is Lys(DOTA).

In another embodiment of the above formulae, X₁₀ is (4-carbamoyl-Phe), 20HPhe, 3FY, 3OHPhe, 3PyA, 4FPhe, Ala, alloThr, aMeLeu, aMeTyr, Aph(Cbm), Cha, Chg, CyanoButric, dAla, FSY, HomoArg, HomoGlu, hyVal, Nle, NMeTyr, OMeTyr, Tba, ThpA, Thr, and Tyr. In yet another embodiment, X₁₀ is selected from Tyr, OMeTyr, Aph(Cbm), and Nle. In still another embodiment, X₁₀ is Tyr or Aph(Cbm). In an embodiment, X₁₀ is Tyr or Aph(Cbm). In another embodiment, X₁₀ is Tyr. In yet another embodiment, X₁₀ is Aph(Cbm). In some embodiments of the above formulae, X₁₀ can be substituted with a chelator, wherein the chelator is optionally linked to the cyclic peptide via a linking group. In an embodiment, X₁₀ is selected from Lys(DOTA), MAF(DOTA), and AEF(DOTA).

In another embodiment of the above formulae, X₁₂ is selected from (2S,3S-betaMe-5FW), 1Nal, 2MeW, 2Nal, 4CIW, 4FW, 4MeOW, 4MeW, 5CIW, 5FW, 5MeOW, 5MeW, 7NW, Ala, aMeW, dAla, hF, HomoArg, HomoGlu, Trp(CH₂COOH), Trp(Me), and Trp. In yet another embodiment, X₁₂ is selected from Trp, Lys(DOTA), 5FW, and 1Nal. In still another embodiment, X₁₂ is selected from 1Nal, 2MeW, 2Nal, 4CIW, 4FW, 4MeOW, 4MeW, 5CIW, 5FW, 5MeOW, 5MeW, 7NW, aMeW, Trp(CH₂COOH), Trp(Me), and Trp. In an embodiment, X₁₂ is selected from Trp, 5FW, and 1Nal. In another embodiment, X₁₂ is 5FW or 1Nal. In another embodiment, X₁₂ is 5FW.

In yet another embodiment of the above formulae, X₁₃ is selected from [(2S, 3R)beta-Me-Phe], [(2S, 3R)beta-OH-Phe], [(2S, 3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, ChA, dAla, Phe, His, HomoArg, HomoGlu, HomoPhe, Leu, Nle, NMeNle, NMePhe, Tbg, and tBuAla. In still another embodiment, X₁₃ is selected from [(2S, 3R)beta-Me-Phe], [(2S, 3R)beta-OH-Phe], [(2S, 3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, aMeNle, aMePhe, Phe, HomoPhe, and NMePhe. In an embodiment, X₁₃ is selected from [(2S, 3R)beta-Me-Phe], [(2S, 3R)beta-OH-Phe], [(2S, 3S)beta-Me-Phe], 2FPhe, 2MePhe, 3ClPhe, and Phe. In another embodiment, X₁₃ is selected from Phe, Nle, and [(2S, 3S)beta-Me-Phe]. In yet another embodiment, X₁₃ is Phe or [(2S, 3S)beta-Me-Phe]. In yet another embodiment, X₁₃ is [(2S, 3S)beta-Me-Phe].

In still another embodiment of the above formulae, X₁₄ is selected from Pro, alpha-Me-Pro, trans4Fluoro-Pro, cis4Fluoro-Pro, trans4OH-Pro, cis4OH-Pro, 5,5-diMe-Pro, trans4NH2-Pro, cis4NH2-Pro, 4PipAla, alloThr, aMeSer, Dab, Dap, dGlu, dLys, dThr, Glu, gE, hydroxyVal, Lys, NMeSer, Orn, Ser, and Thr. In an embodiment of the above formulae, X₁₄ is selected from Pro, alpha-Me-Pro, trans4Fluoro-Pro, cis4Fluoro-Pro, trans4OH-Pro, cis4OH-Pro, 5,5-diMe-Pro, trans4NH2-Pro, cis4NH2-Pro, hydroxyVal, and alloThr.

In another embodiment of the above formulae, X₁₄ is selected from hydroxyVal, Pro, and Thr. In yet another embodiment of the above formulae, X₁₄ is hydroxyVal or Thr. In yet another embodiment, X₁₄ is hydroxyVal. If X₁₅ is absent, then X₁₄ can be substituted with a C-terminus group selected from P². In an embodiment of the above formulae, X₁₄ is absent.

In another embodiment of the above formulae, X₁₅ is selected from dAla, dLys, dSer, dGlu, Gly, and betaAla. In yet another embodiment, X₁₅ is selected from dAla, dSer, and dGlu. In yet another embodiment, X₁₅ is dGlu. In still another embodiment of the above formulae, X₁₅ is substituted with a C-terminus group selected from P². In an embodiment, X₁₅ is absent. If X₁₄ is absent, then X₁₅ is absent.

In another aspect, provided herein is a cyclic peptide of Formula I:

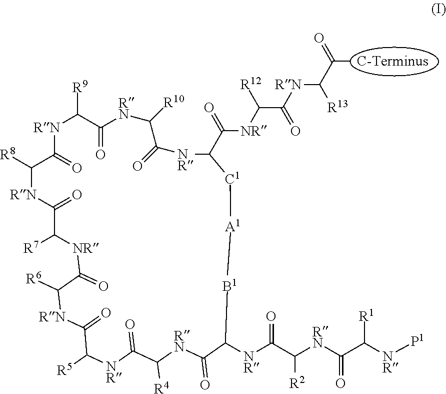

or a pharmaceutically acceptable salt thereof,
wherein:
the C-Terminus is selected from R, N(R″)P²,

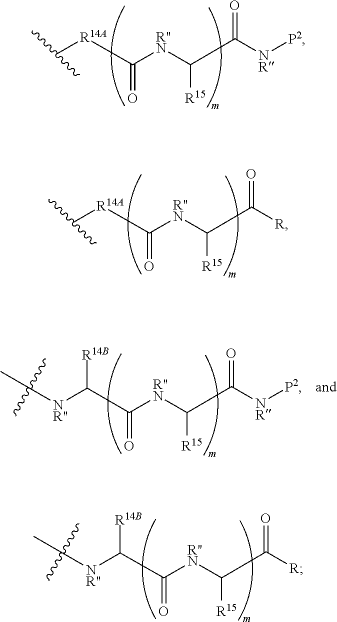

P¹ is selected from R″, —SO₂R″,

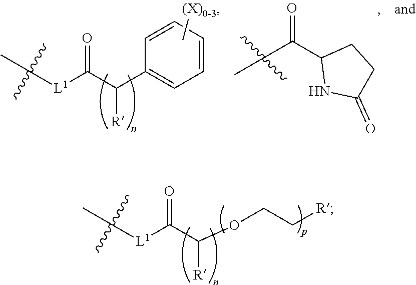

L¹ is absent or selected from:

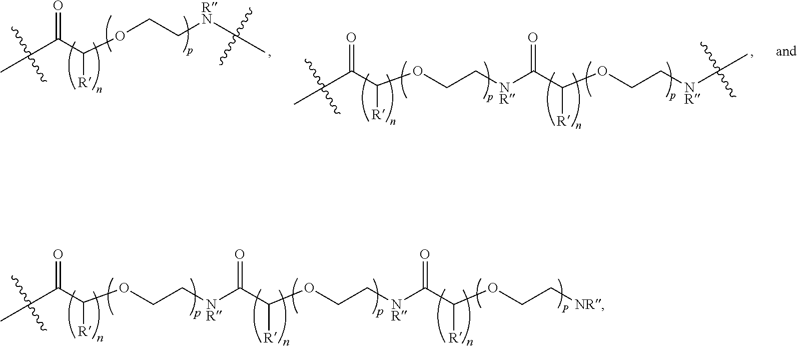

wherein the amino group of L¹ connects to the carbonyl group of P¹ to form an amide bond;
P² is selected from R″,

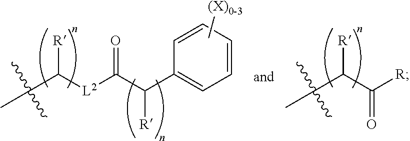

L² is absent or is selected from:

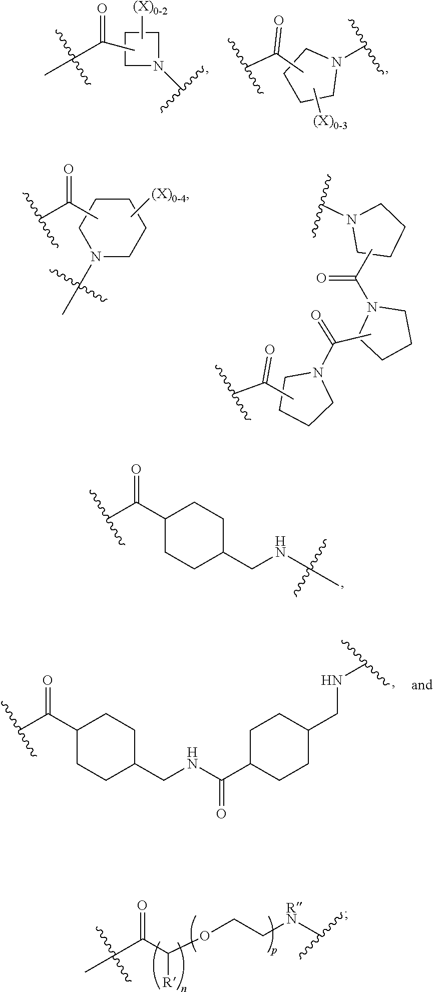

wherein the amino group of L² connects to the carbonyl group of P^{2′} to form an amide bond;
R¹ is an amino acid side chain of a natural amino acid or an amino acid side chain of an unnatural amino acid;
R² is an amino acid side chain of a natural amino acid or an amino acid side chain of an unnatural amino acid;
B¹ is C₁-6 alkylene;
C¹ is C₁-6 alkylene;
A¹ is selected from:

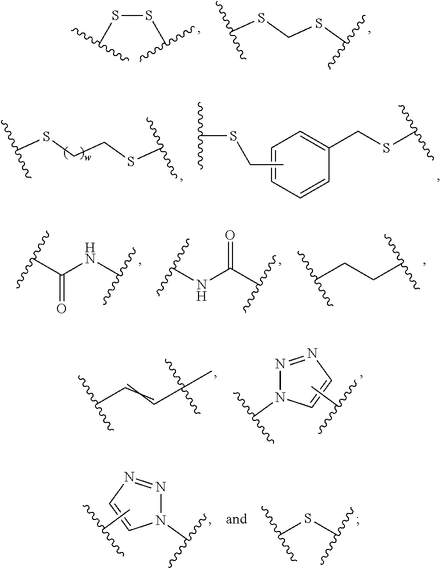

w is 1, 2, or 3;
R⁴ is selected from an amino acid side chain of a natural amino acid or an amino acid side chain of an unnatural amino acid;
R⁵ is selected from:
(i) an amino acid side chain of a natural amino acid,
(ii) an amino acid side chain of an unnatural amino acid,
(iii) L³-L³-Chelator,

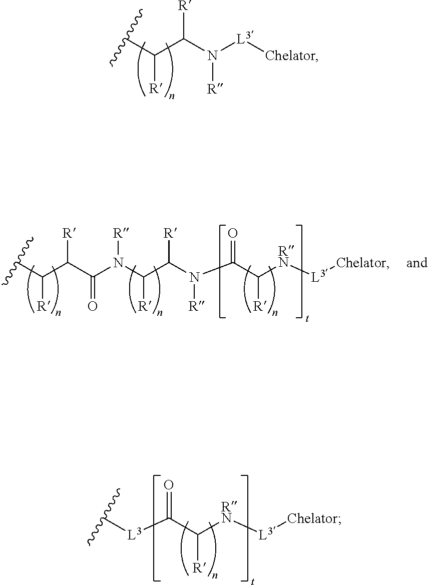

R⁶ is selected from:
(i) an amino acid side chain of a natural amino acid,
(ii) an amino acid side chain of an unnatural amino acid,
(iii) L³-L^{3′}-Chelator,

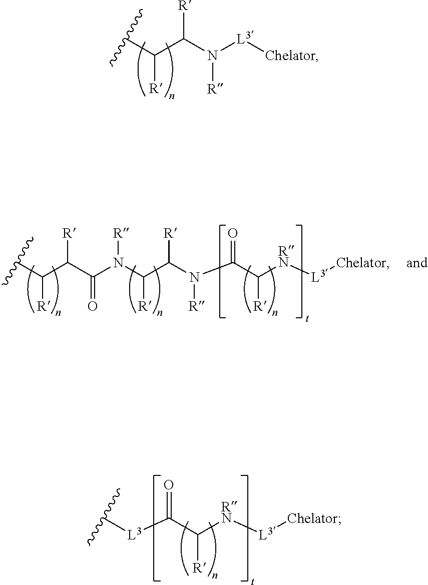

R⁷ is selected from:
(i) an amino acid side chain of a natural amino acid,
(ii) an amino acid side chain of an unnatural amino acid,
(iii) L³-L³-Chelator,

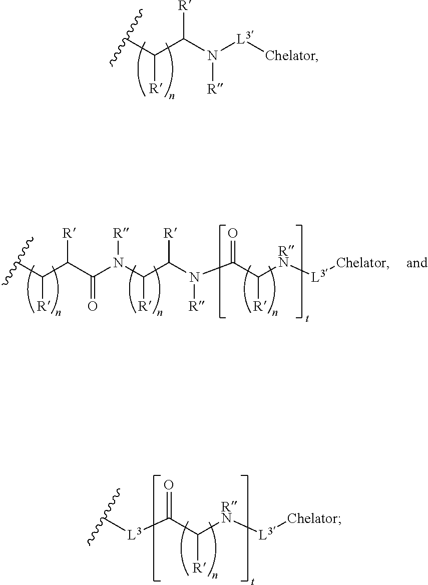

R⁸ is selected from an amino acid side chain of a natural amino acid or an amino acid side chain of an unnatural amino acid;
R⁹ is selected from:
(i) an amino acid side chain of a natural amino acid,
(ii) an amino acid side chain of an unnatural amino acid,
(iii) L³-L^{3′}-Chelator,

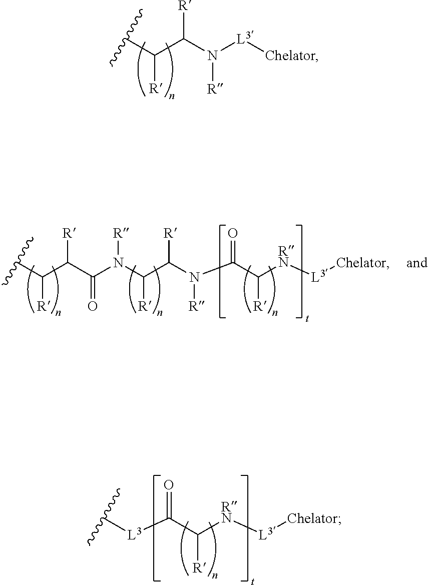

R¹⁰ is selected from:
(iii) an amino acid side chain of a natural amino acid,
(ii) an amino acid side chain of an unnatural amino acid,
(iii) L³-L³-Chelator,

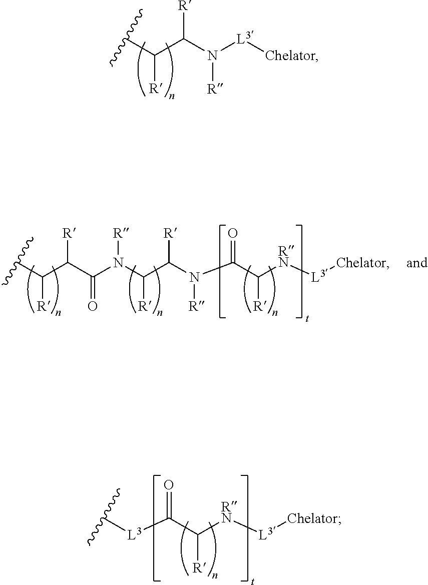

R¹² is selected from an amino acid side chain of a natural amino acid or an amino acid side chain of an unnatural amino acid;
R¹³ is selected from an amino acid side chain of a natural amino acid or an amino acid side chain of an unnatural amino acid;
R^{14A} is selected from:

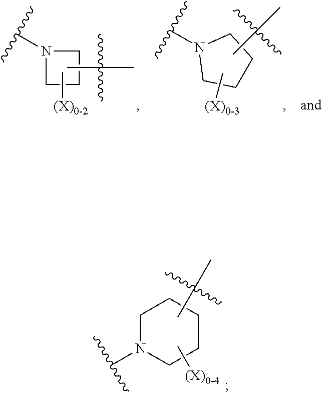

R^{14B} is selected from an amino acid side chain of a natural amino acid or an amino acid side chain of an unnatural amino acid;
R¹⁵ is an amino acid side chain of a natural amino acid or an amino acid side chain of an unnatural amino acid;
L³ is absent or selected from

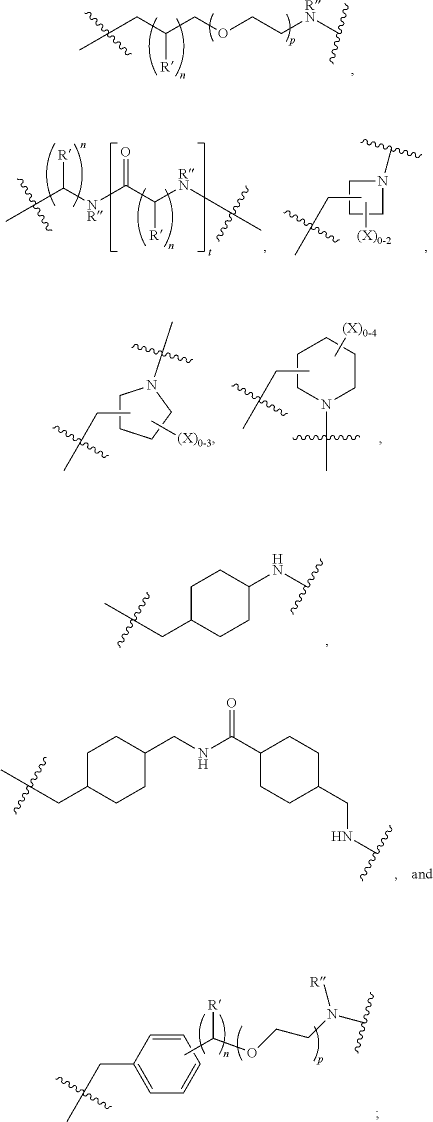

L^{3′} is absent or selected from

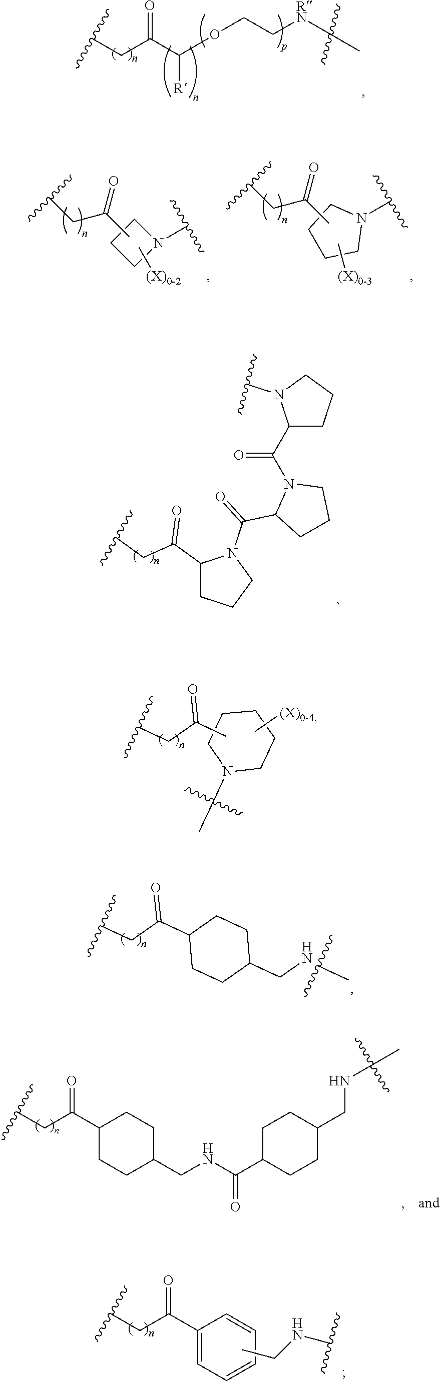

m is 0 or 1;
each n is independently an integer from 0 to 16;
each p is independently an integer from 0 to 30;
each t is independently 0, 1, 2, 3, 4, 5, or 6;
X is independently, for each occurrence, selected from halo, OH, C₁-C₆ alkyl, C₁-C₆ haloalkyl, N(R″)₂, —O—(C₁-C₆ alkyl);
R is independently, for each occurrence, selected from H, C₁-C₂₀ alkyl, C₁-C₆ haloalkyl, OH, —O—(C₁-C₆ alkyl), (C₁-C₆ alkyl)-OH, and N(R″)₂;
R′ is independently, for each occurrence, selected from H, OH, C₁-C₆ alkyl, C₁-C₆ haloalkyl, C₁-C₈ alkylamine, C₁-C₈ alkyl-OH, C(O)H, C(O)(C₁-C₆ alkyl), C(O)OH, C(O)O(C₁-C₆ alkyl), (C₁-C₆ alkyl)C(O)OH, (C₁-C₆ alkyl)C(O)O(C₁-C₆ alkyl), (C₁-C₆ alkyl)CO(C₁-C₆ alkyl), (C₁-C₆ alkyl)CS(C₁-C₆ alkyl), C(O)N(R″)₂, N(R″)₂, and N(R″)₃⁺; and R″ is independently, for each occurrence, selected from H, C₁-C₆ alkyl, C₁-C₆ haloalkyl, (C₁-C₆ alkyl)C(O)OH, (C₁-C₆ alkyl)C(O)N(alkyl)₂, and C(O)O(C₁-C₆ alkyl);
wherein the cyclic peptide comprises one Chelator;
wherein when a variable group R¹, R², R⁴, R⁵, R⁶, R⁷, R⁸, R⁹, R¹⁰, R¹², R¹³, or R¹⁵ is the amino acid side chain of a cyclic amino acid, the corresponding amino acid nitrogen of the peptide backbone forms part of the cyclic group;
each alpha-carbon atom on the peptide backbone is optionally substituted with methyl; and
wherein the cyclic peptide optionally comprises a radionuclide.

In some embodiments, the cyclic peptides provided herein are cyclic peptides of Formula I, or a pharmaceutically acceptable salt and/or solvate thereof.

In an embodiment, the cyclic peptide of Formula I is a cyclic peptide of Formula l′:

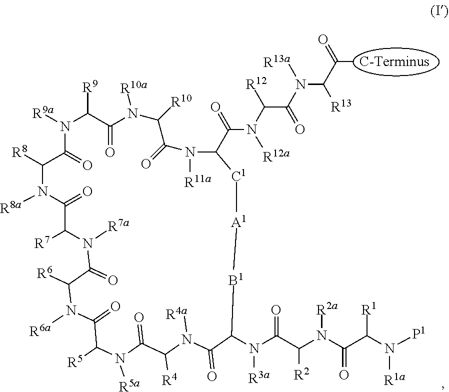

or a pharmaceutically acceptable salt and/or solvate thereof;
wherein
the C-terminus is selected from R, N(R″)P²,

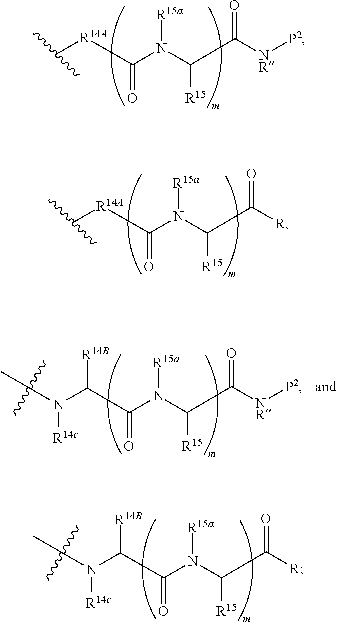

wherein each R^{1a}, R^{2a}, R^{3a}, R^{4a}, R^{5a}, R^{6a}, R^{7a}, R^{8a}, R^{9a}, R^{10a}, R^{11a}, R^{12a}, R^{13a}, R^{15a}, or R^{14c}, is, independently for each occurrence, selected from H and CH₃. In an embodiment, 0, 1, 2, 3, or 4 of R^{1a}, R^{2a}, R^{3a}, R^{4a}, R^{5a}, R^{6a}, R^{7a}, R^{8a}, R^{9a}, R^{10a}, R^{11a}, R^{12a}, R^{13a}, R^{15a}, or R^{14c}, are CH₃.

In an embodiment, the cyclic peptide of Formula I is a cyclic peptide of Formula IIa:

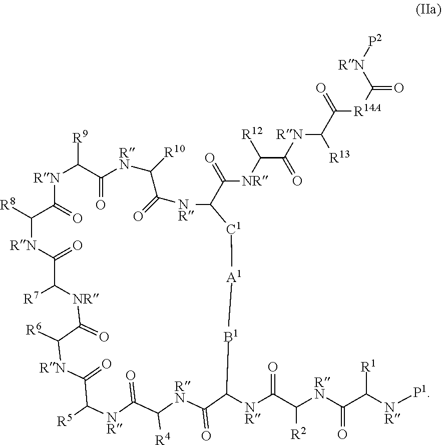

In some embodiments, the cyclic peptides provided herein are cyclic peptides of Formula IIa, or a pharmaceutically acceptable salt and/or solvate thereof.

In another embodiment, the cyclic peptide of Formula I is a cyclic peptide of Formula IIb:

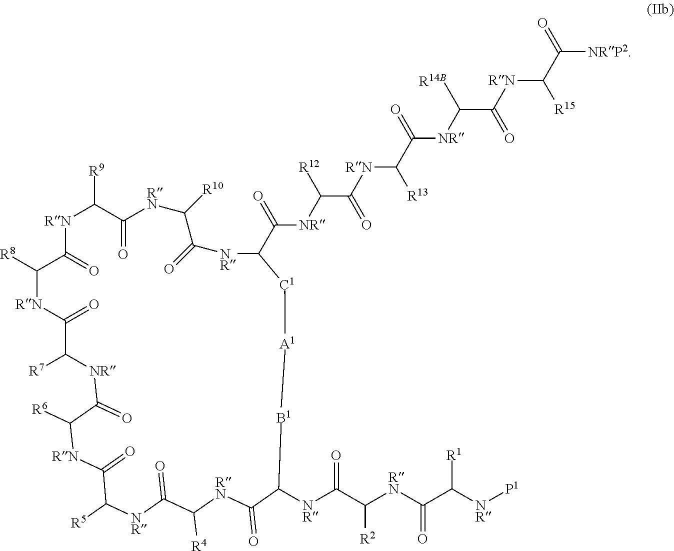

In some embodiments, the cyclic peptides provided herein are cyclic peptides of Formula IIb, or a pharmaceutically acceptable salt and/or solvate thereof.

In yet another embodiment, the cyclic peptide of Formula I is a cyclic peptide of Formula IIc:

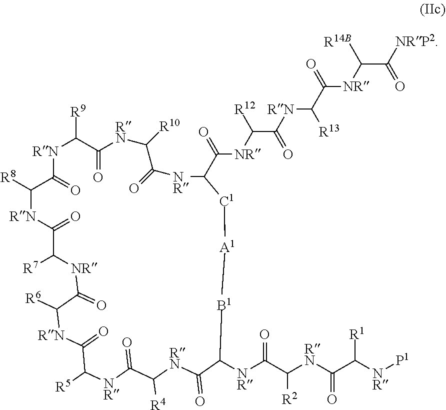

In some embodiments, the cyclic peptides provided herein are cyclic peptides of Formula IIc, or a pharmaceutically acceptable salt and/or solvate thereof.

In still another embodiment, the cyclic peptide of Formula I is a cyclic peptide of Formula IIIa:

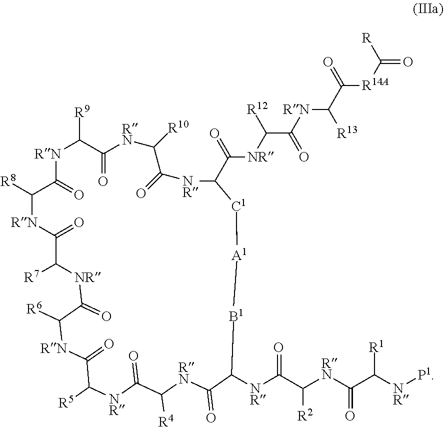

In some embodiments, the cyclic peptides provided herein are cyclic peptides of Formula IIIa, or a pharmaceutically acceptable salt and/or solvate thereof.

In an embodiment, the cyclic peptide of Formula I is a cyclic peptide of Formula IIIb:

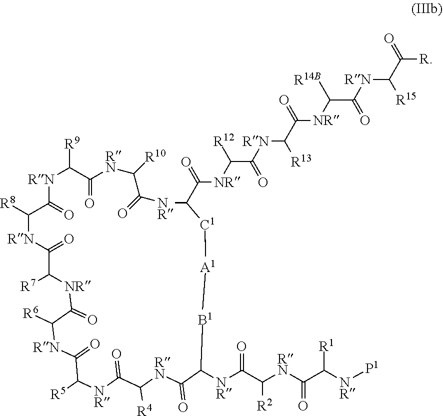

In some embodiments, the cyclic peptides provided herein are cyclic peptides of Formula IIIb, or a pharmaceutically acceptable salt and/or solvate thereof.

In another embodiment, the cyclic peptide of Formula I is a cyclic peptide of Formula IIIc:

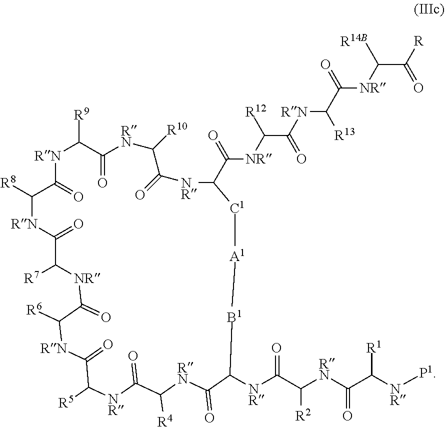

In some embodiments, the cyclic peptides provided herein are cyclic peptides of Formula IIIc, or a pharmaceutically acceptable salt and/or solvate thereof.

In yet another embodiment of the above formulae, P¹ is selected from —SO₂R″,

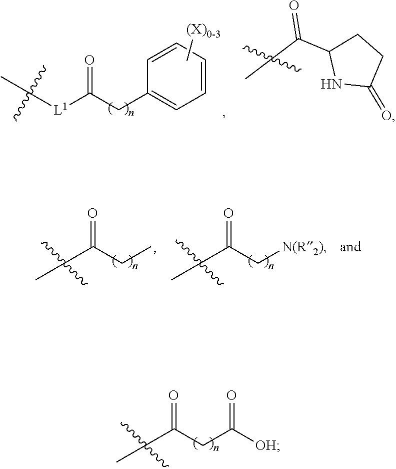

n is an integer from 0 to 15; and
R″ is H or C₁-C₆ alkyl.

In still another embodiment of the above formulae, P¹ is selected from —SO₂R′,

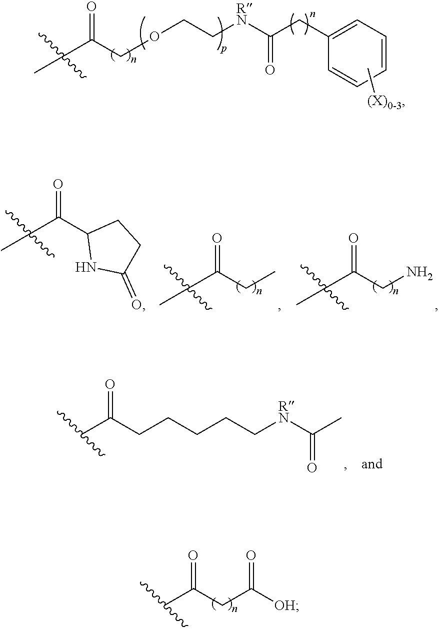

R′ is H, C₁-C₆ alkyl, C(O)(C₁-C₆ alkyl), C(O)OH, (C₁-C₆ alkyl)C(O)OH, and C(O)N(R″)₂;
R″ is independently, for each occurrence, H or C₁-C₆ alkyl;
n is an integer from 1 to 10; and
p is an integer from 1 to 12.

In an embodiment of the above formulae, P¹ is selected from —SO₂Me,

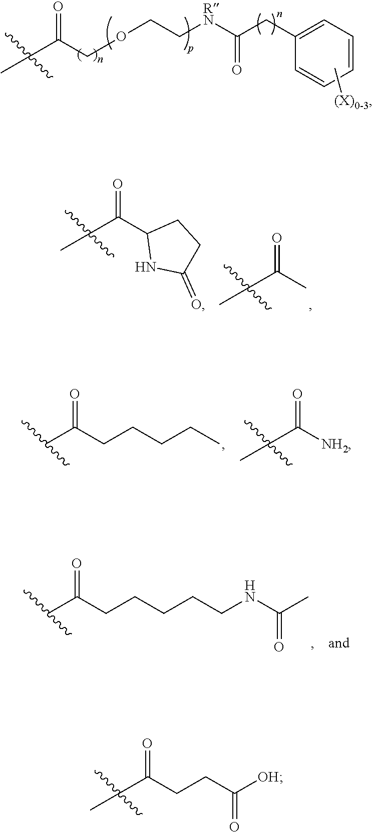

R″ is H or C₁-C₆ alky;
each n is independently an integer from 1 to 4; and
p is an integer from 1 to 12.

In another embodiment of the above formulae, the C-Terminus is selected from OH, N(R″)₂,

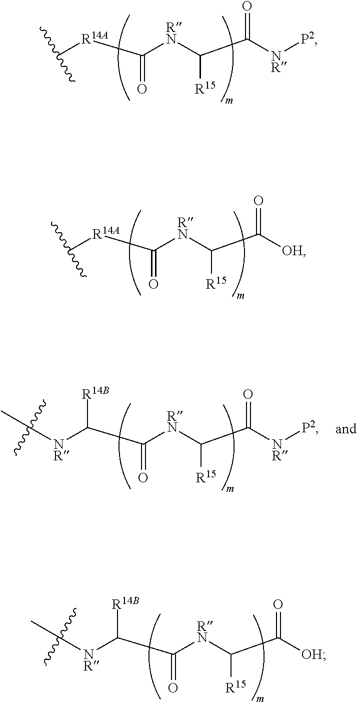

P² is selected from R″,

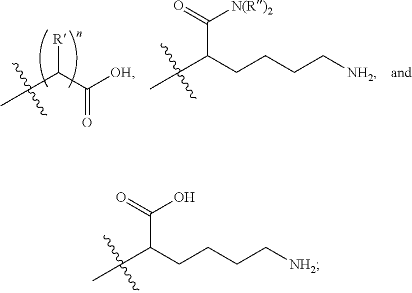

R′ is H or C₁-C₈ alkyl-OH;
R″ is independently, for each occurrence, H or C₁-C₆ alkyl;
each m is independently an integer from 0 to 1; and
n is an integer from 1 to 6.

In yet another embodiment of the above formulae, L³ is absent or selected from

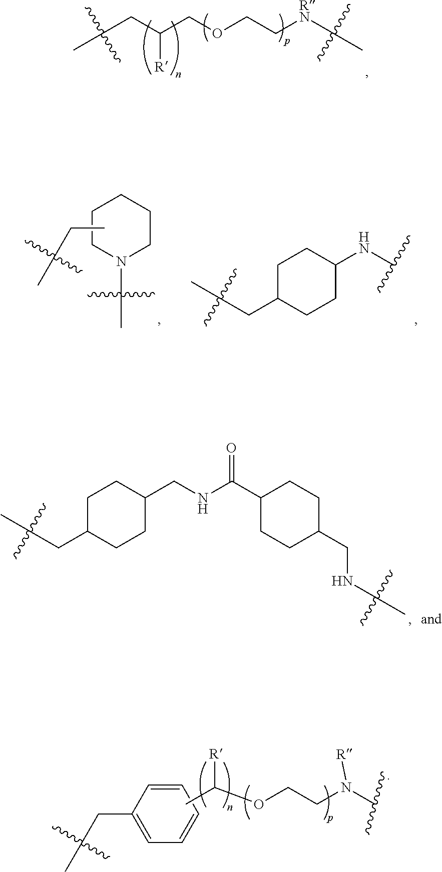

In still another embodiment of the above formulae, L^{3′} is absent or selected from

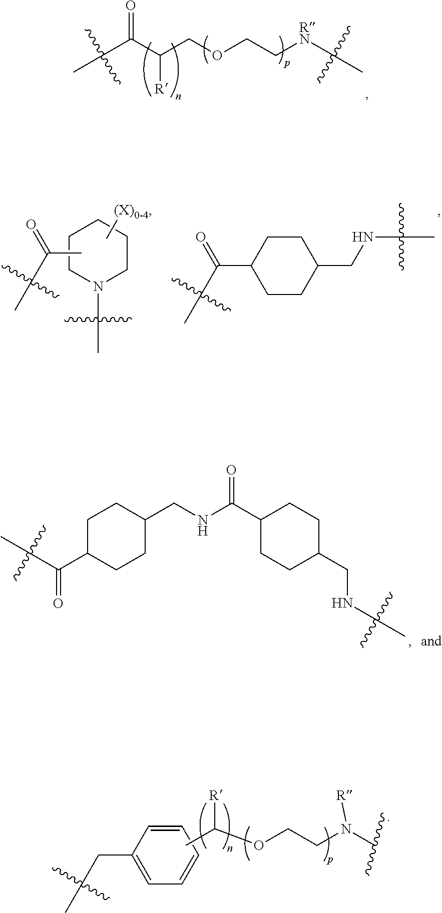

In an embodiment of the above formulae, one of R⁵, R⁶, R⁷, R⁹, and R¹⁰ is selected from

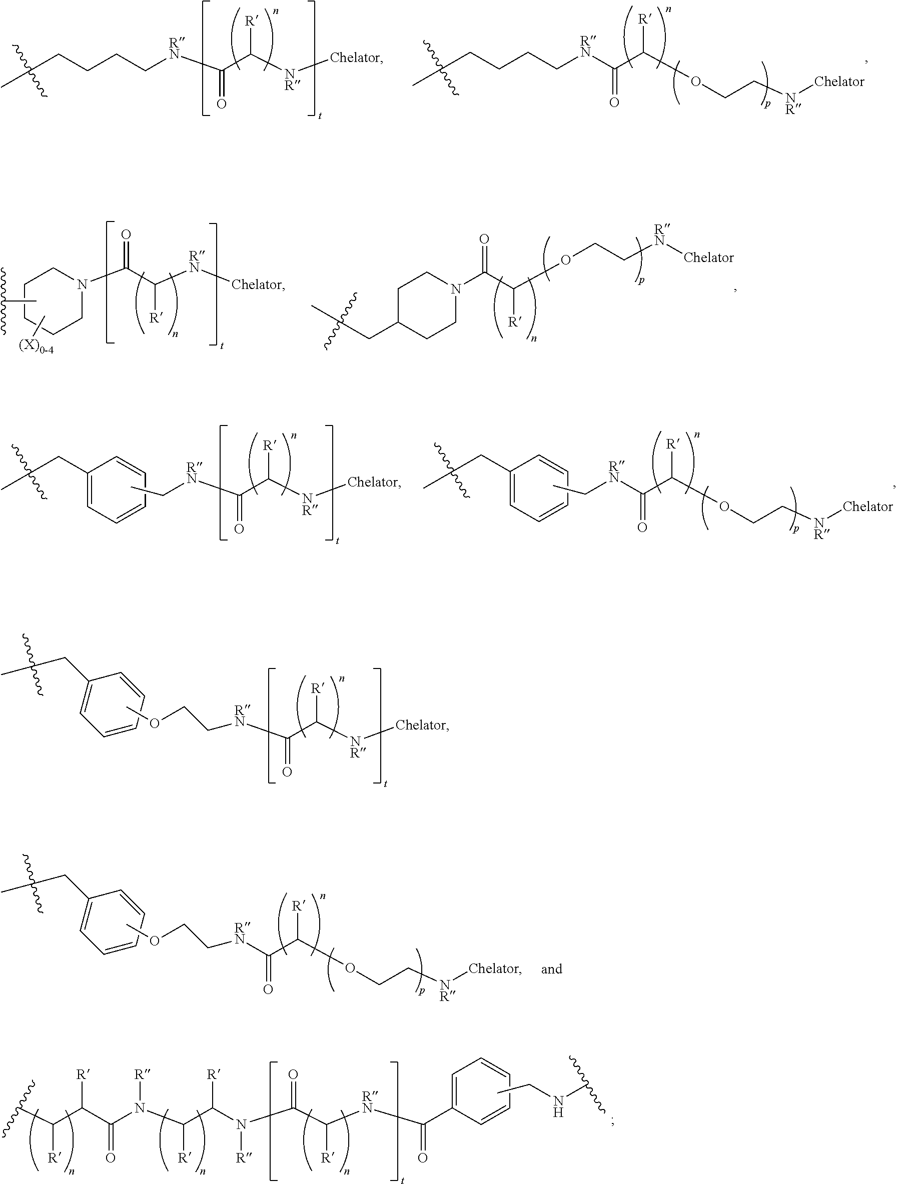

wherein R′ is selected from H, C(O)OH, (C₁-C₆ alkyl)C(O)OH, C₁-C₆ alkyl, and (C₁-C₆ alkyl)S(C₁-C₆ alkyl);
R″ is H or C₁-C₆ alkyl;
each n is independently an integer from 1 to 8;
each p is independently an integer from 0 to 12; and
each t is independently 0, 1, 2, or 3.

In an embodiment of the above formulae, one of R⁵, R⁶, R⁷, R⁹, and R¹⁰ is selected from

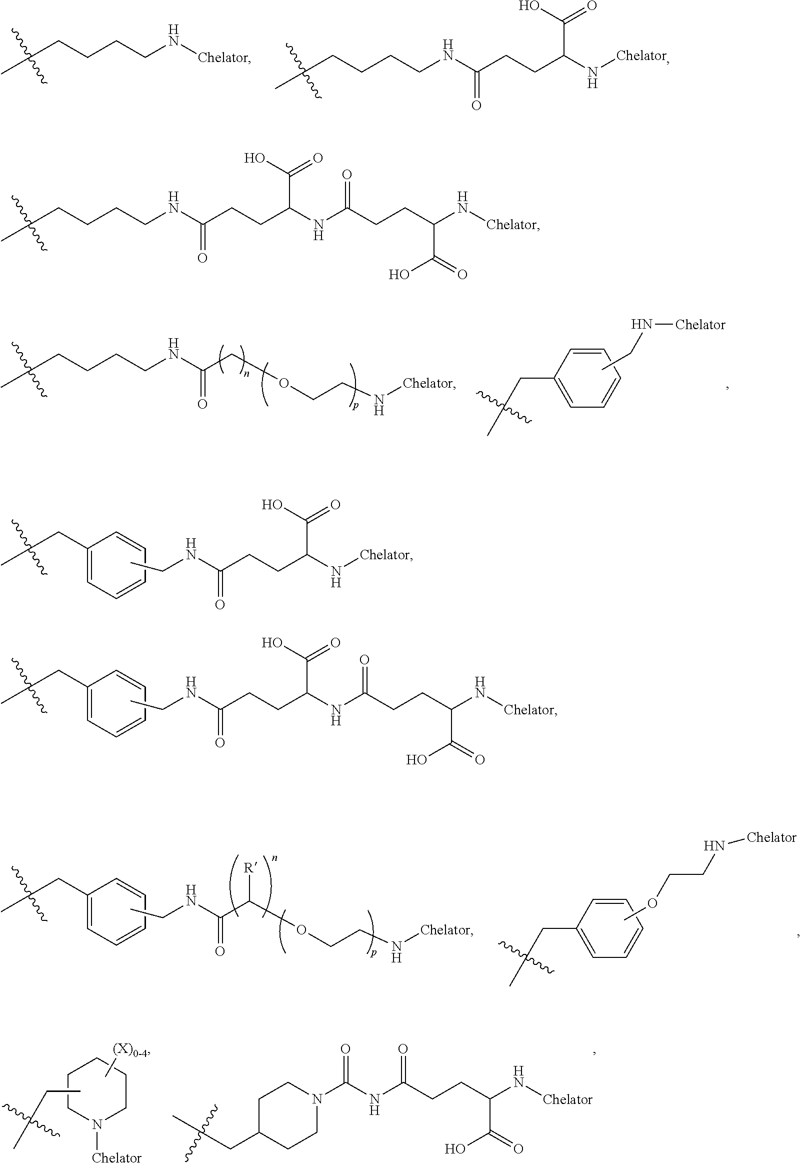

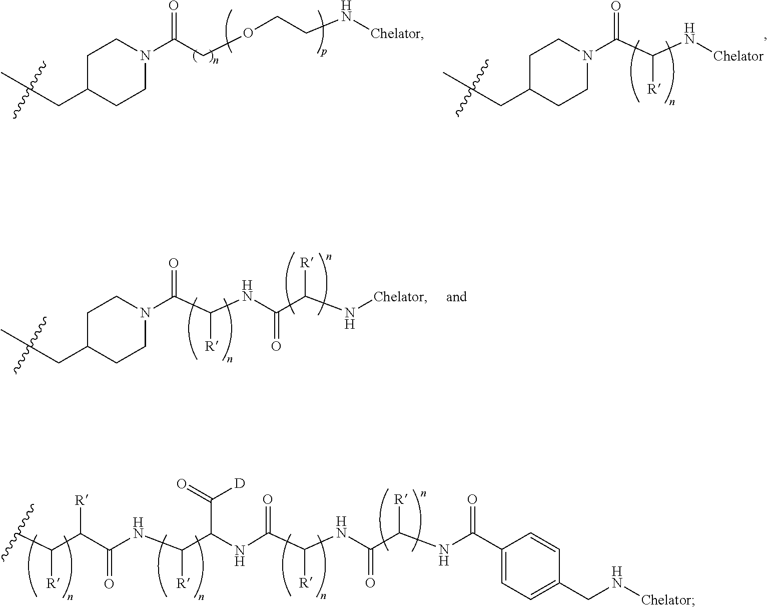

wherein each R′ is independently selected from H, C(O)OH, C₁-C₆ alkyl, and (C₁-C₆ alkyl)S(C₁-C₆ alkyl);
each n is independently an integer from 1 to 8; and
each p is independently an integer from 0 to 12.

In another embodiment, the cyclic peptide of Formula I is a cyclic peptide of Formula IVa:

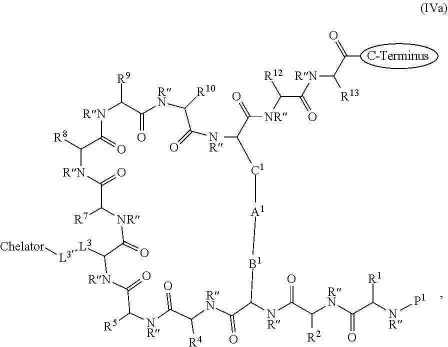

or a pharmaceutically acceptable salt and/or solvate thereof.

In another embodiment, the cyclic peptide of Formula I is a cyclic peptide of Formula IVb:

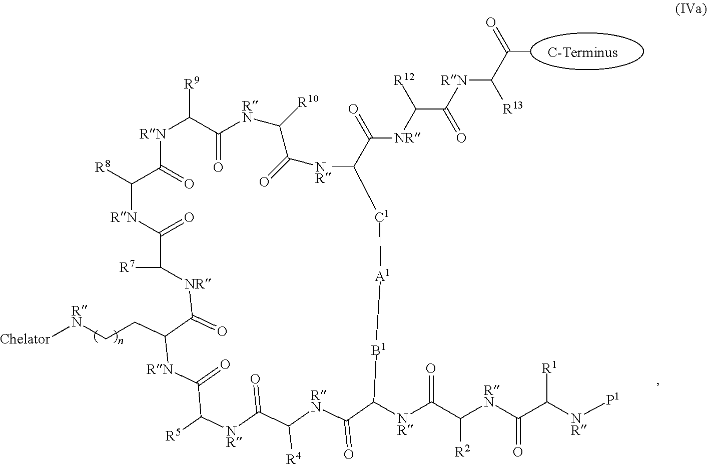

or a pharmaceutically acceptable salt and/or solvate thereof.

In another embodiment, the cyclic peptide of Formula I is a cyclic peptide of Formula IVc:

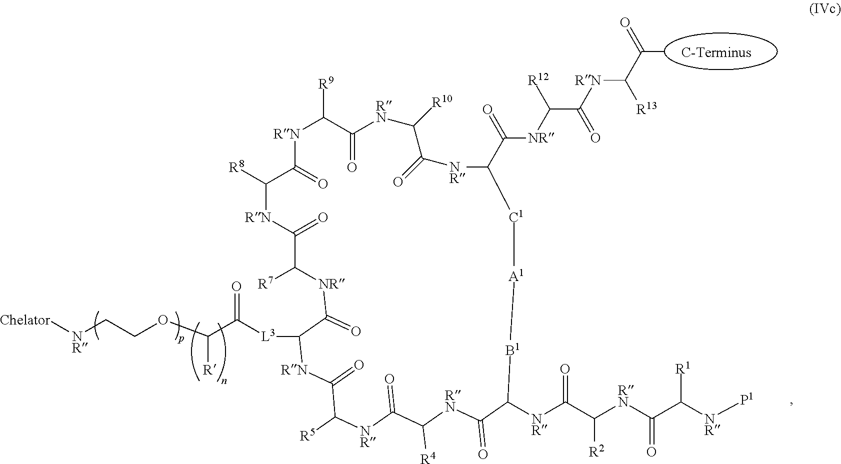

or a pharmaceutically acceptable salt and/or solvate thereof.

In another embodiment, the cyclic peptide of Formula I is a cyclic peptide of Formula IVd:

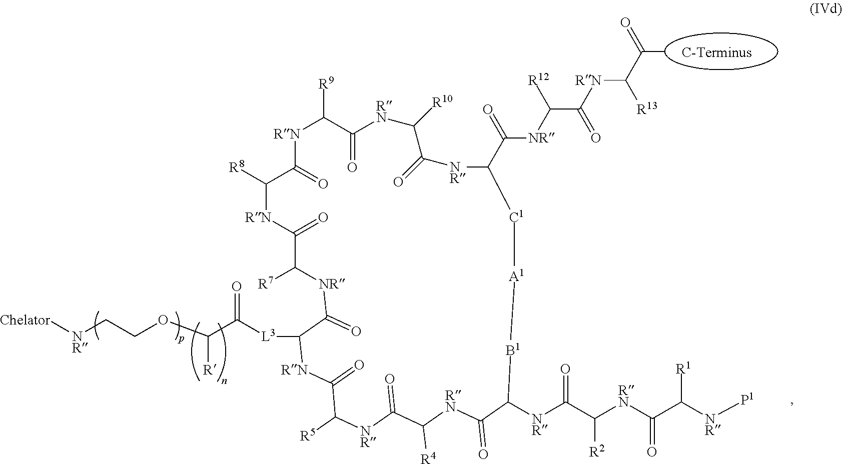

or a pharmaceutically acceptable salt and/or solvate thereof.

In another embodiment, the cyclic peptide of Formula I is a cyclic peptide of Formula Va:

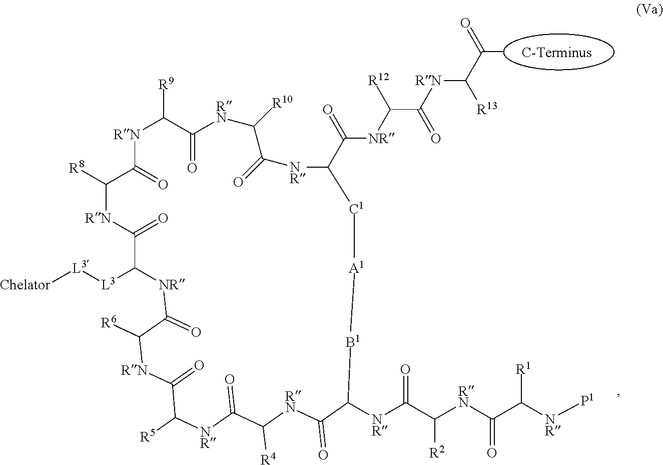

or a pharmaceutically acceptable salt and/or solvate thereof.

In another embodiment, the cyclic peptide of Formula I is a cyclic peptide of Formula Vb:

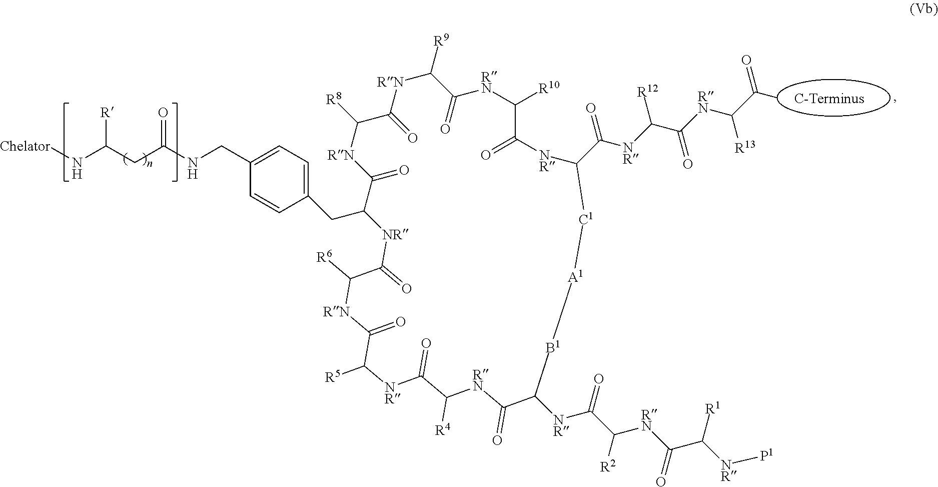

or a pharmaceutically acceptable salt and/or solvate thereof.

In another embodiment, the cyclic peptide of Formula I is a cyclic peptide of Formula Vc:

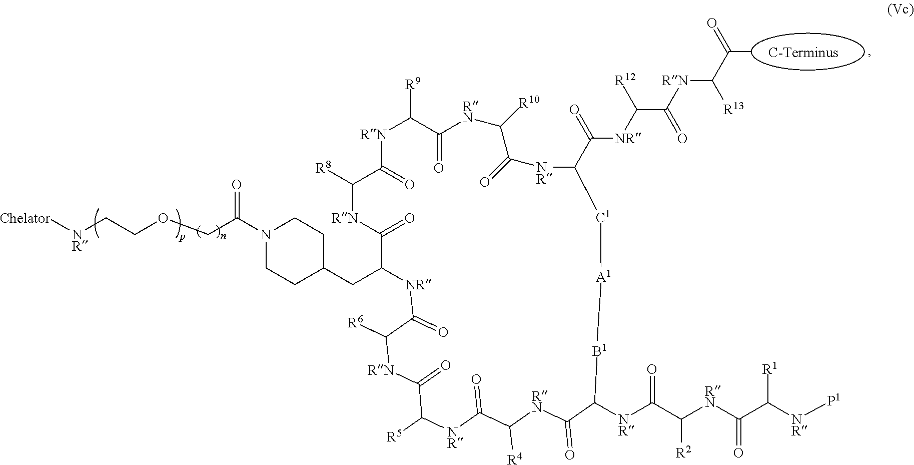

or a pharmaceutically acceptable salt and/or solvate thereof.

In another embodiment, the cyclic peptide of Formula I is a cyclic peptide of Formula Vd:

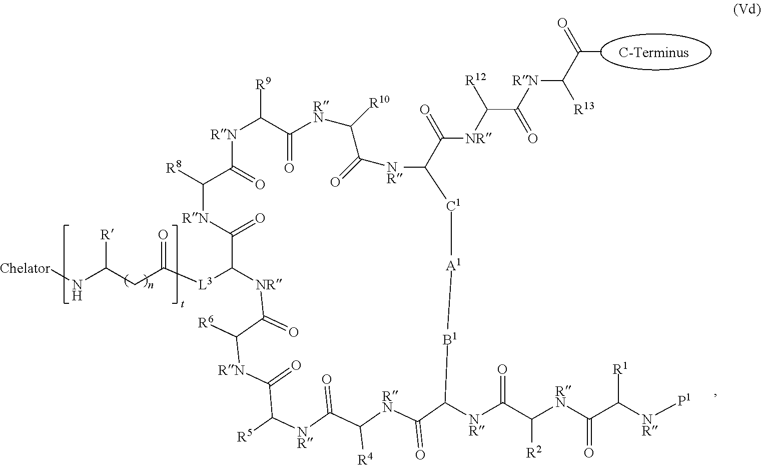

or a pharmaceutically acceptable salt and/or solvate thereof.

In another embodiment, the cyclic peptide of Formula I is a cyclic peptide of Formula Ve:

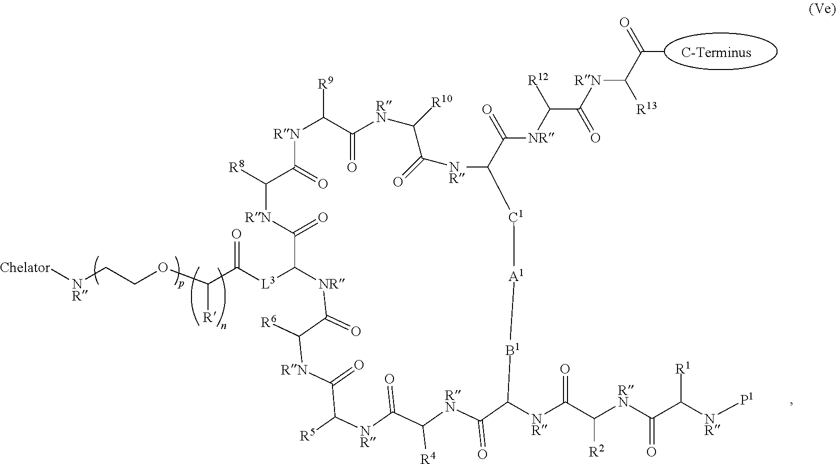

or a pharmaceutically acceptable salt and/or solvate thereof.

In another embodiment of the above formulae, R¹ is selected from the group consisting of an amino acid side chain of Ala, Cha, dAla, hCba, hCha, hLeu, HomoArg, HomoGlu, hTba, Leu, Met, and Nle;
R² is selected from the group consisting of an amino acid side chain of 3CIY, 3FY, 4COOHPhe, 4FPhe, 4PyA, 7AzaW, Ala, alloThr, aMeNle, aMeTyr, Cba, Chg, dAla, FSY, HomoArg, HomoGlu, hTyr, hyVal, Nle, NMeNle, OMeTyr, Tba, ThpA, ThpG, Thr, and Tyr;
R⁴ is selected from the group consisting of an amino acid side chain ofAbu, allolle, aMeVal, alloThr, betaMelle, ChG, Cpg, dAla, HomoGlu, hyVal, Ile, Nle, NMeNle, NMeVal, Nva, PipAla, Tbg, tBuAla, Thr, and Val;
R⁵ is:
(i) selected from the group consisting of an amino acid side chain of Ala, dAla, Gly, HomoGlu, N(CH2)2COOHGly, NetGly, Ser, or Sar; or
(ii) selected from

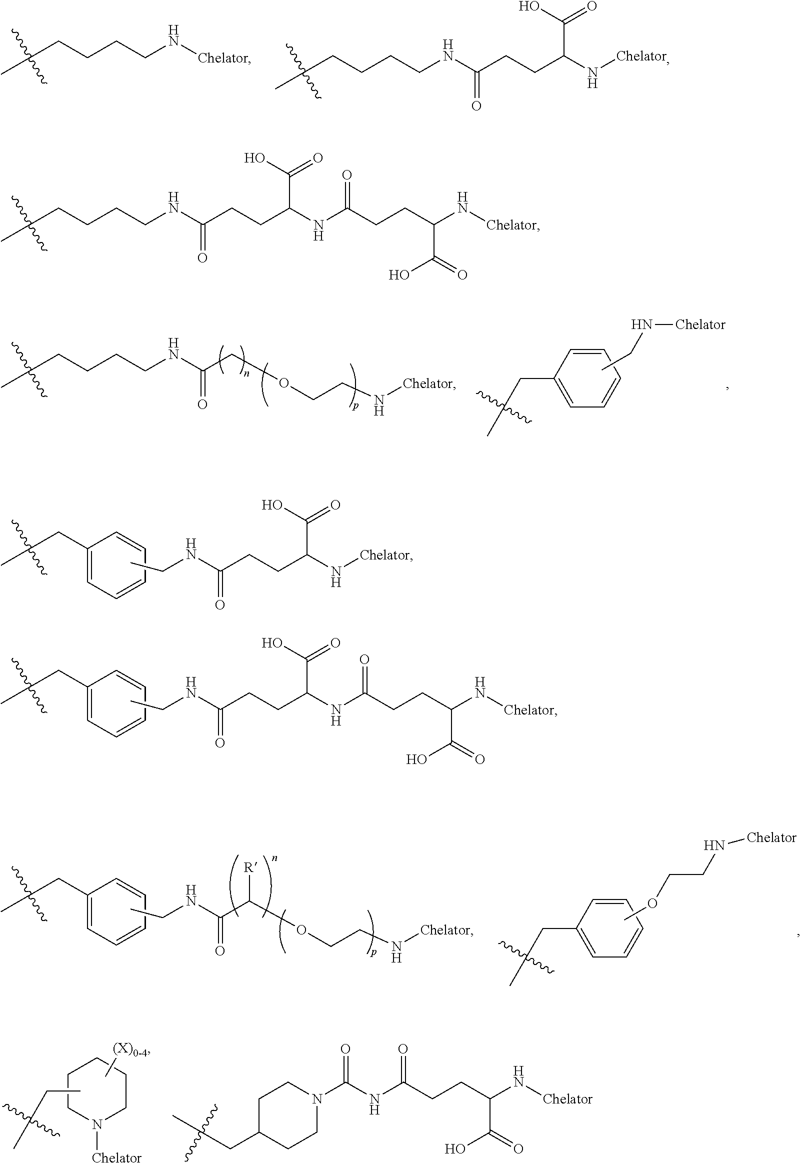

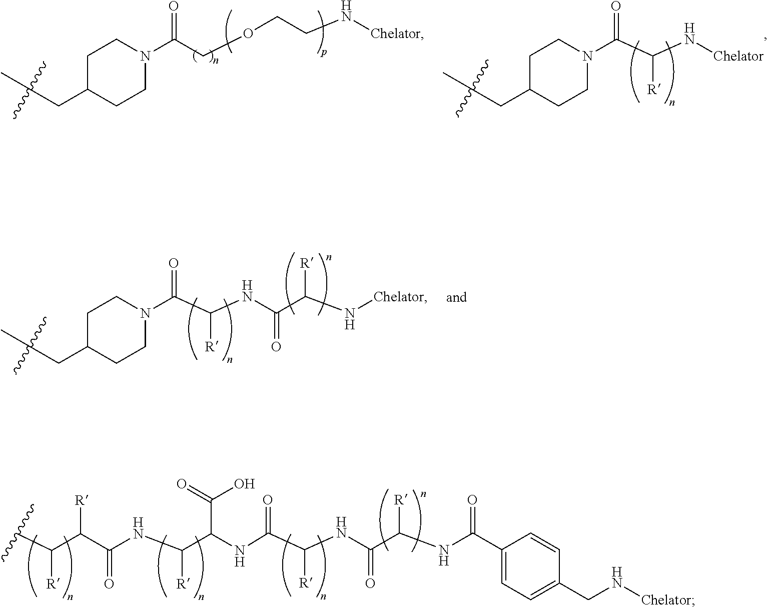

R⁶ is:
(i) selected from the group consisting of an amino acid side chain of Ala, aMeLys, Arg, Aze, Cit, Dab, dAla, Dap, dPro, HomoGlu, hSer, Lys, Lys(Ac), Lys(Me)2, Lys(Me)3, Nle, NMeLys, Orn, Pip, Pip(Ac)Ala, Pip(C4diacid)Ala, Pip(CH2COOH)Ala, Pip(CONH₂)Ala, Pip(Me)Ala, PipAla, Pro, ThpA, and 4PyA; or
(ii) selected from the group consisting of:

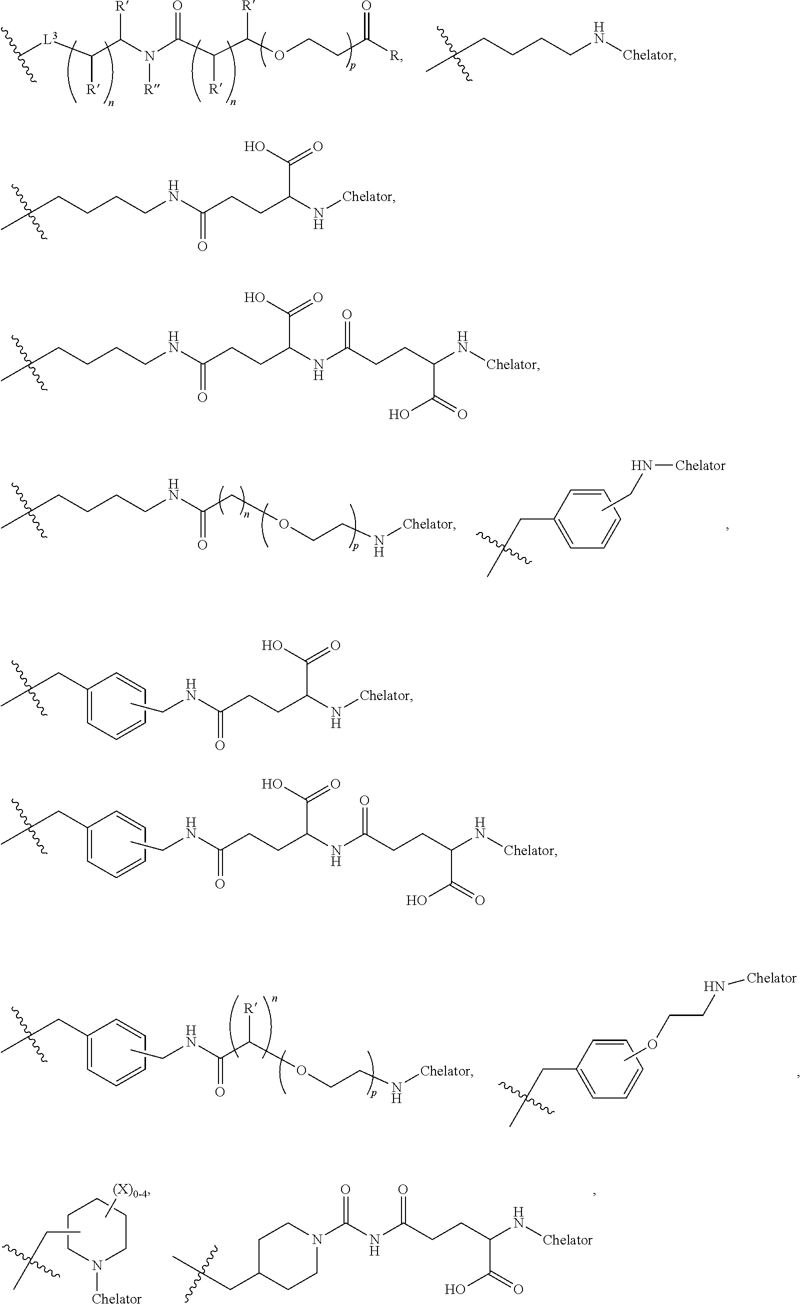

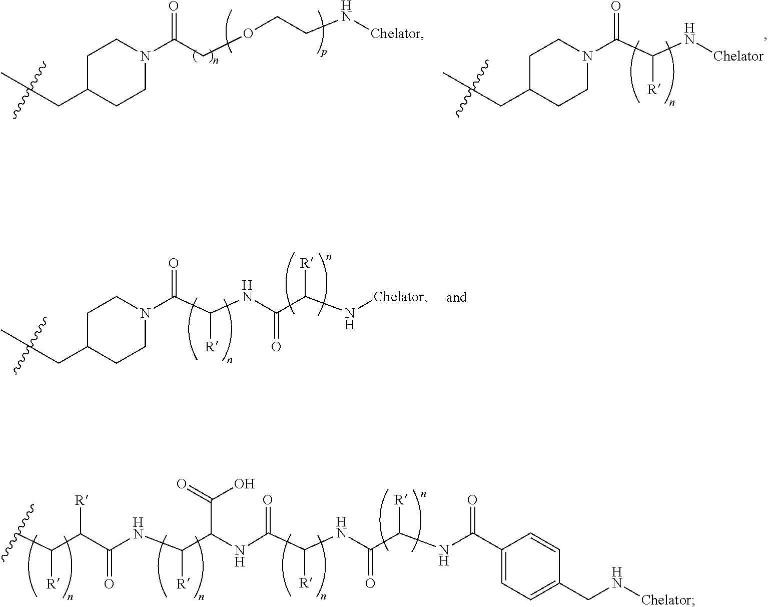

R⁷ is:
(i) selected the group consisting of an amino acid side chain of from (N-Me-ThpA), 4COOHPhe, 4OHCha, 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, and Tyr; or
(ii) selected from the group consisting of:

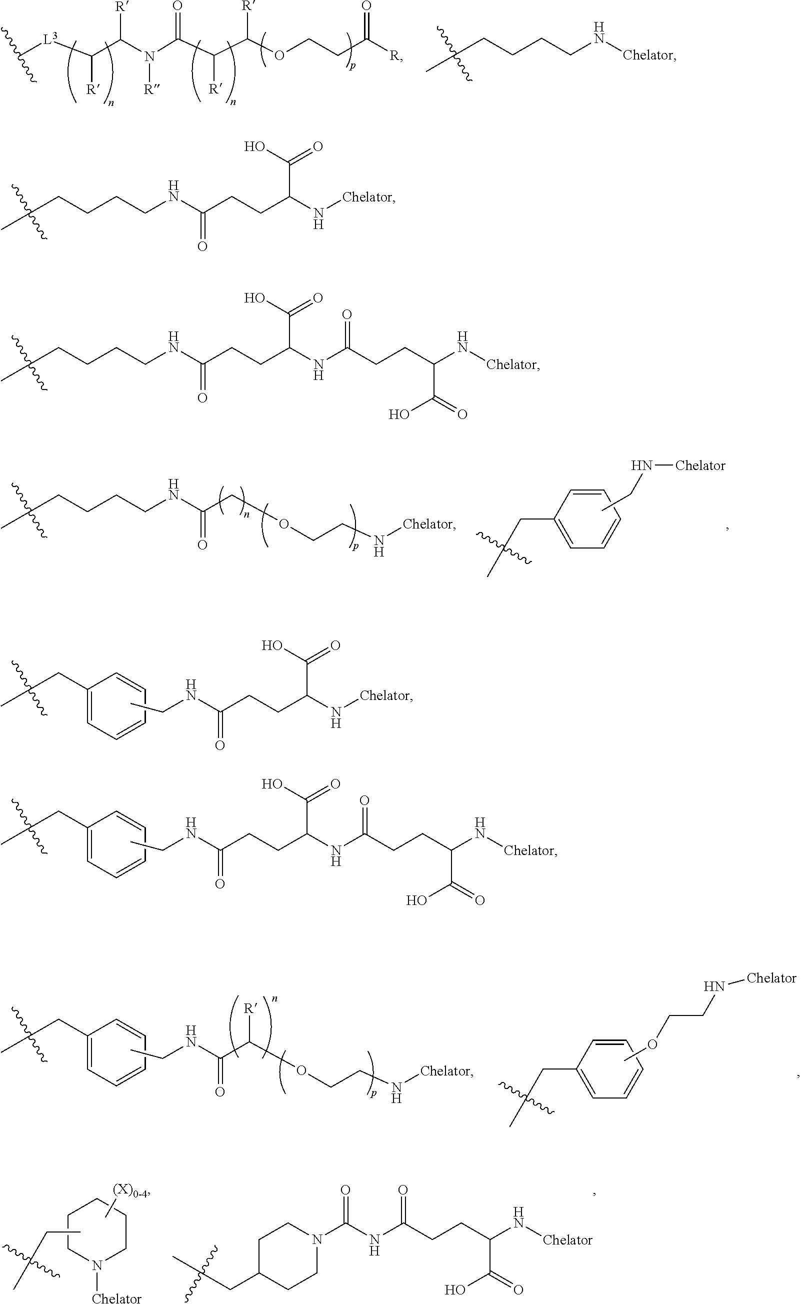

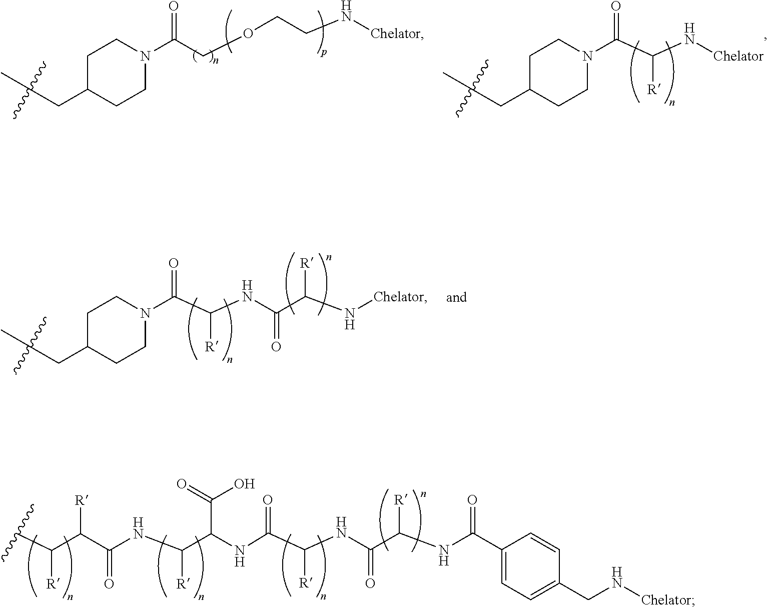

R⁸ is selected from the group consisting of an amino acid side chain of Ala, CysAcid, Asp, Dab, dAla, FSY, HomoGlu, Asn, and Ser;
R⁹ is:
(i) selected from the group consisting of an amino acid side chain of Abu, Aib, Ala, allolle, alloThr, aMeSer, aMeVal, ChG, Cpg, dAla, HomoGlu, hSer, hydroVal, Nle, NMeVal, Nva, Ser, Tbg, Thr, and Val; or
(ii) selected from the group consisting of:

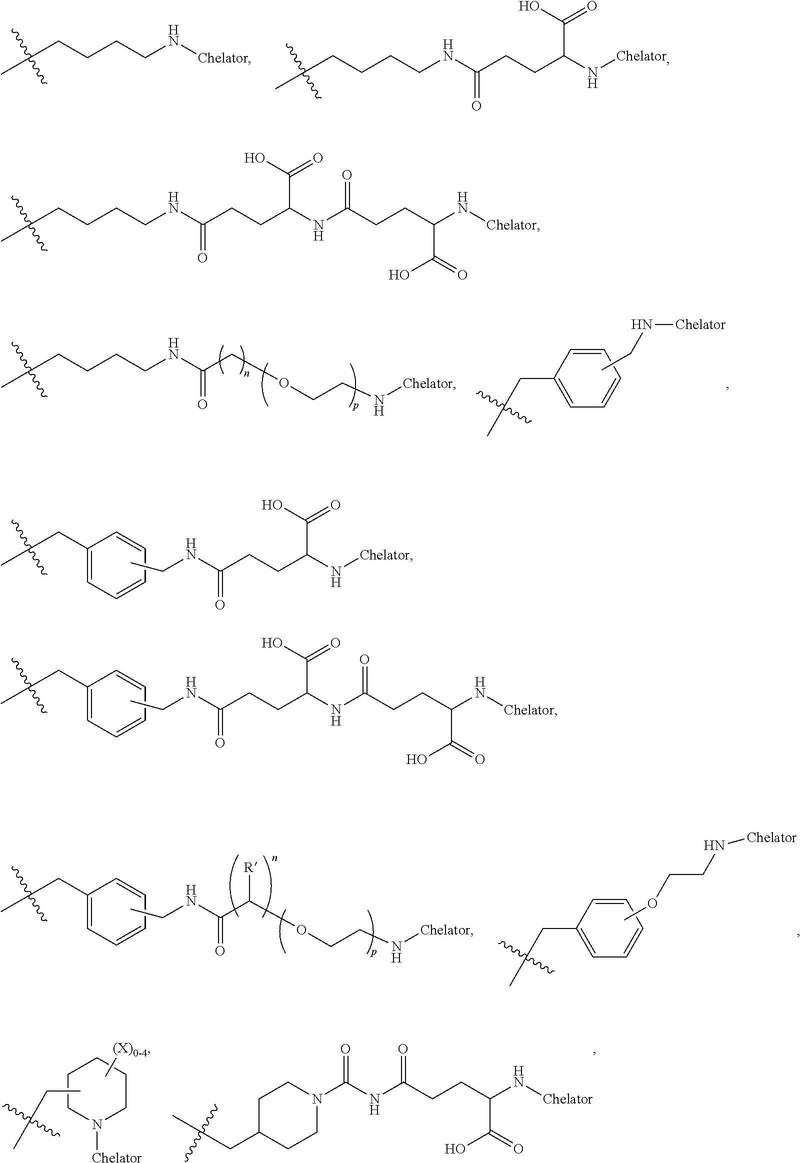

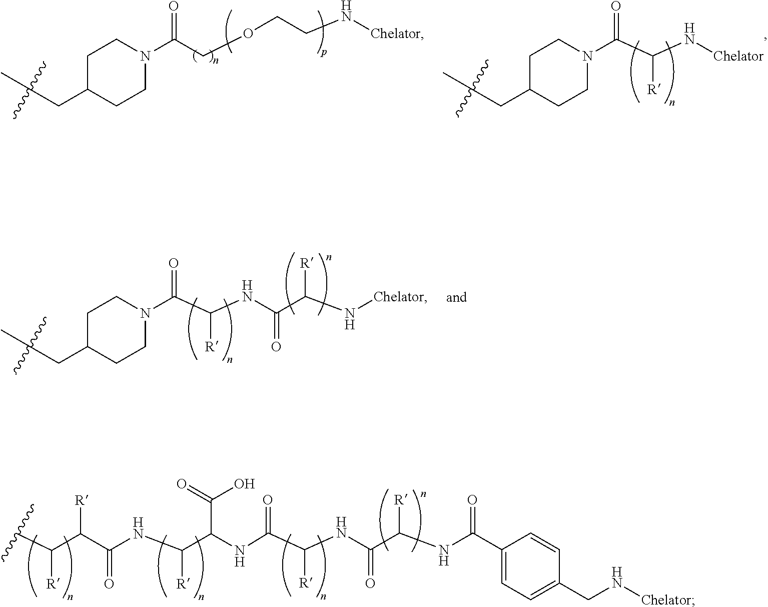

R¹⁰ is:
(i) selected from the group consisting of an amino acid side chain of (4-carbamoyl-Phe), 20HPhe, 3FY, 3OHPhe, 3PyA, 4FPhe, Ala, alloThr, aMeLeu, aMeTyr, Aph(Cbm), Cha, Chg, CyanoButric, dAla, FSY, HomoArg, HomoGlu, hyVal, Nle, NMeTyr, OMeTyr, Tba, ThpA, Thr, and Tyr; or
(ii) selected from the group consisting of:

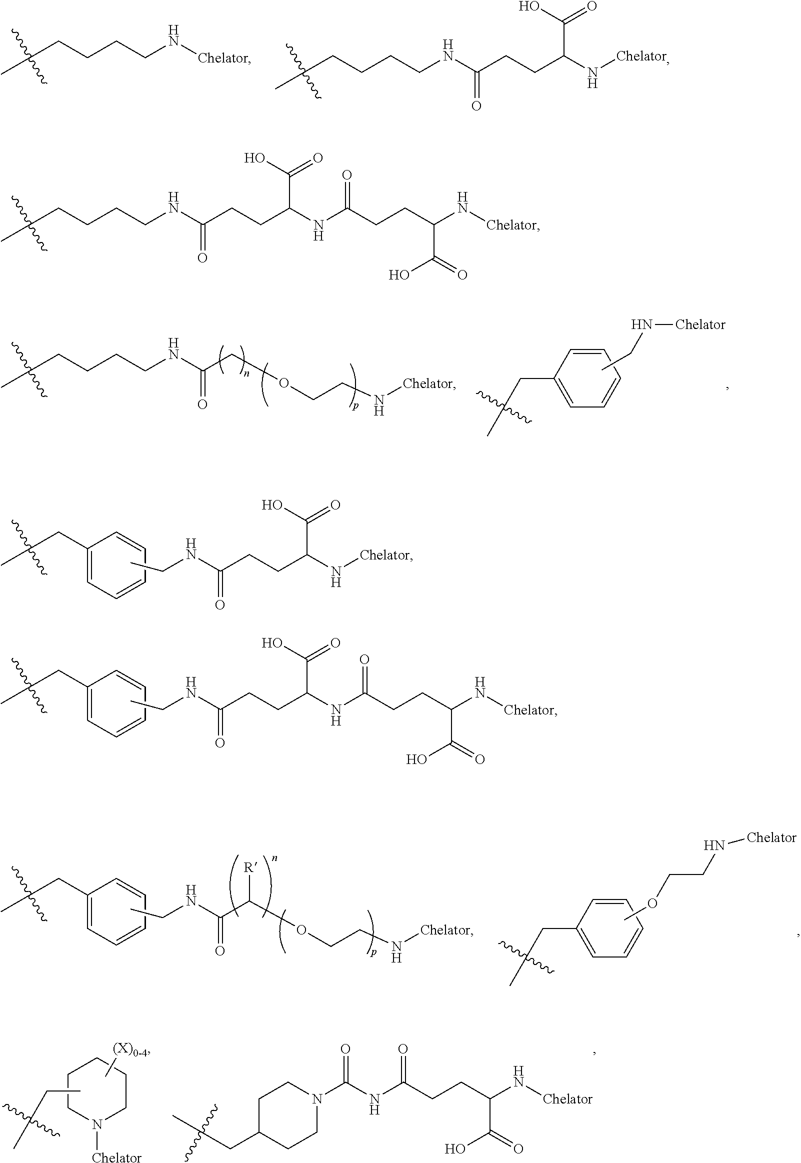

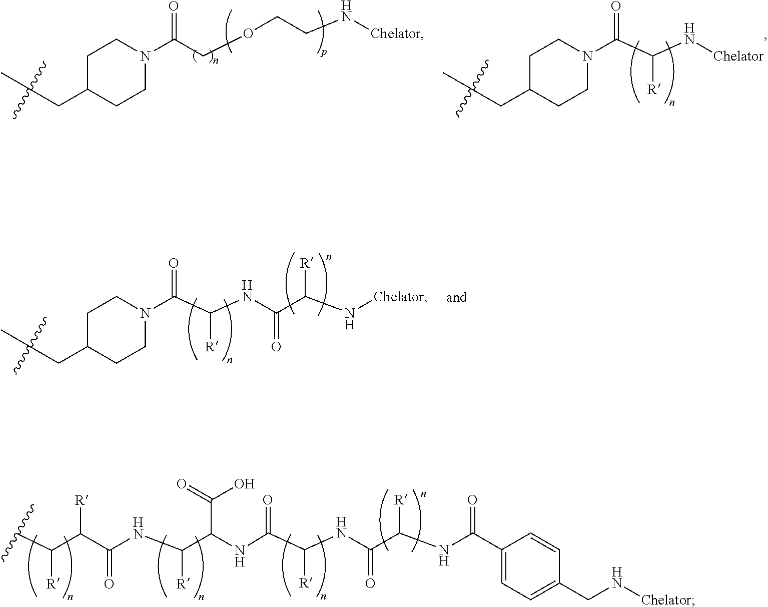

R¹² is selected from the group consisting of an amino acid side chain of (2S,3S-betaMe-5FW), 1Nal, 2MeW, 2Nal, 4CIW, 4FW, 4MeOW, 4MeW, 5CIW, 5FW, 5MeOW, 5MeW 7NW, Ala, aMeW, dAla, hF, HomoArg, HomoGlu, Trp(CH₂COOH), Trp(Me), and Trp; and
R¹³ is selected from the group consisting of an amino acid side chain of [(2S, 3R)beta-Me-Phe], [(2S, 3R)beta-OH-Phe], [(2S, 3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, ChA, dAla, Phe, His, HomoArg, HomoGlu, HomoPhe, Leu, Nle, NMeNle, NMePhe, Tbg, and tBuAla; and
R^{14A} is

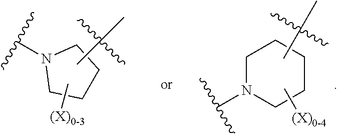

In yet another embodiment, R¹ is selected from the group consisting of an amino acid side chain of hLeu, hCha, hCba and Nle;
R² is an amino acid side chain of Tyr;
R⁴ is selected from the group consisting of an amino acid side chain of Ile, Tbg, and Val;
R⁵ is an amino acid side chain of Gly or Sar;
R⁶ is:
(i) selected from the group consisting of an amino acid side chain of PipAla, Lys, ThpA, Pip(CH2COOH)Ala, Cit, Lys(Ac), Pip(Ac)Ala, Pip(CONH₂)Ala, Pip(Me)Ala, Lys(Me)2, and Lys(Me)3; or
(ii) selected from the group consisting of:

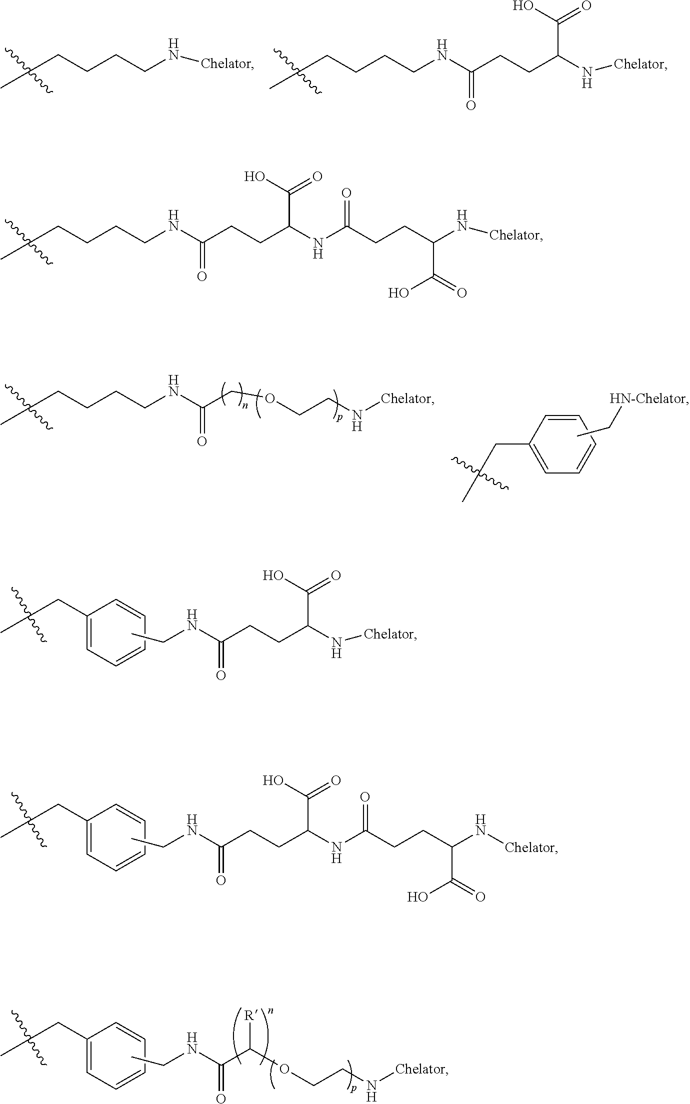

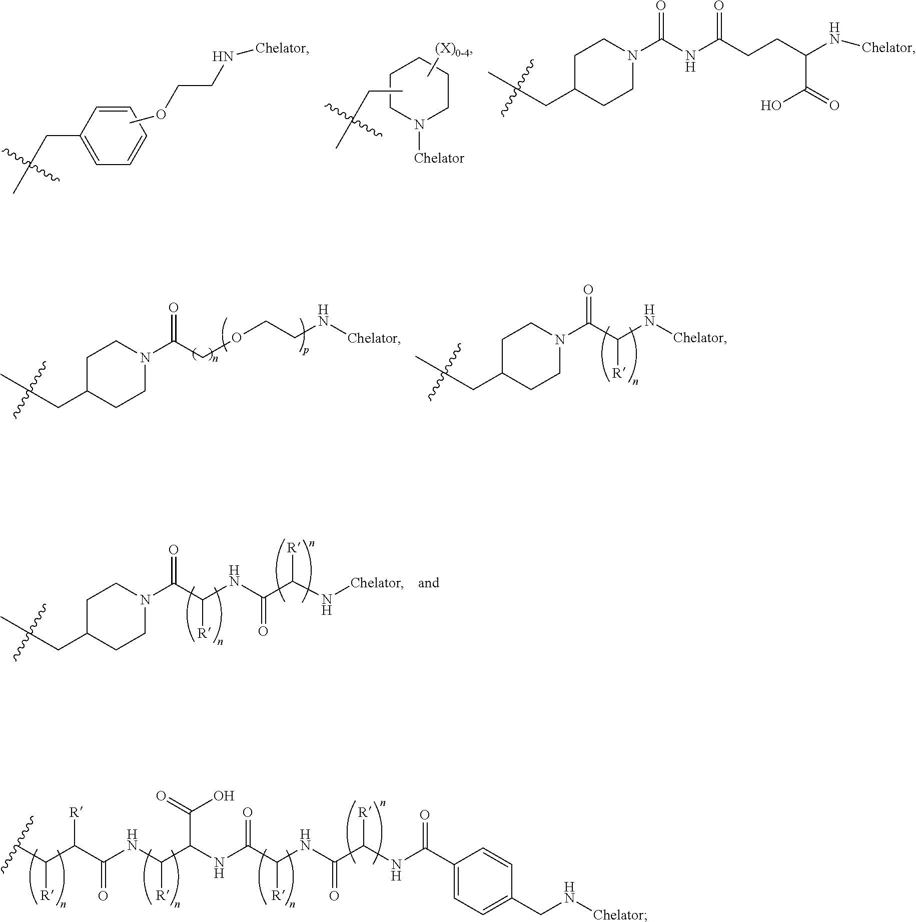

R⁷ is:
(i) selected from the group consisting of an amino acid side chain of 4COOHPhe, 4Pya, PipAla, and ThpA; or
(ii) selected from the group consisting of:

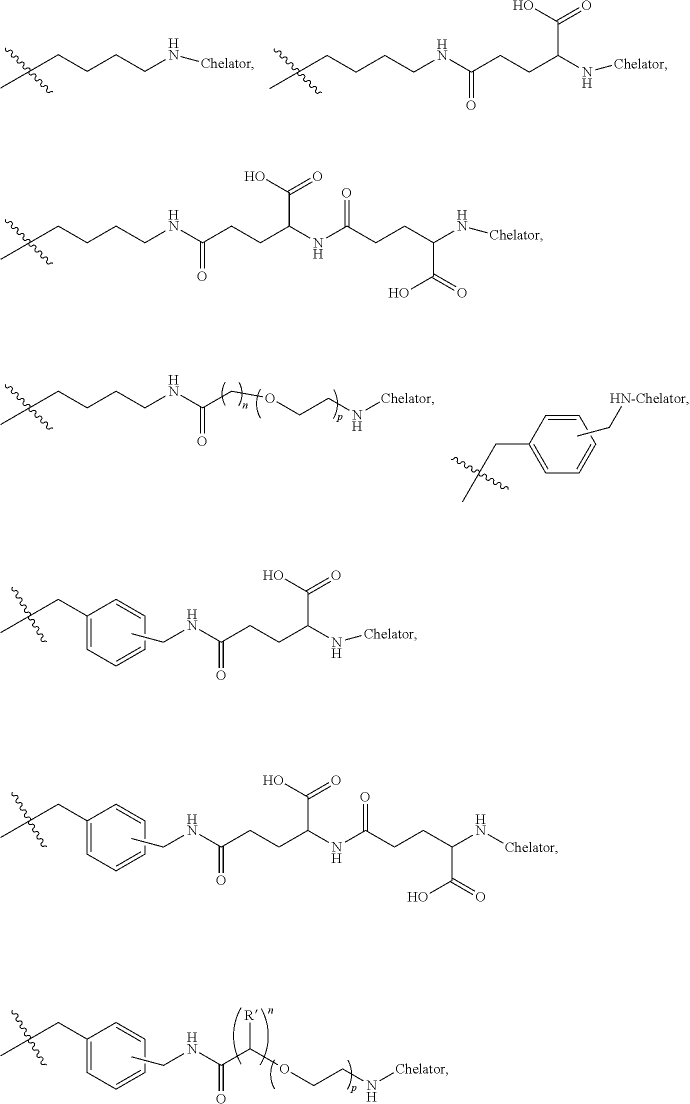

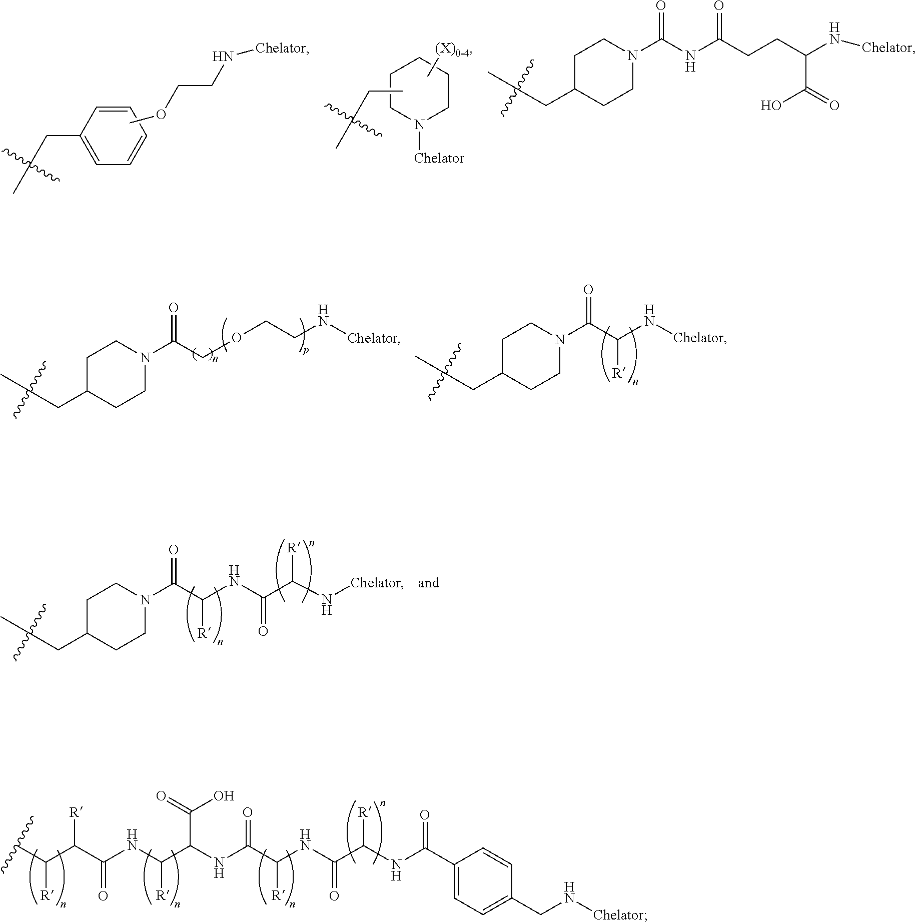

R⁸ is an amino acid side chain of Asp or CysAcid;
R⁹ is an amino acid side chain of Val;
R¹⁰ is an amino acid side chain of Tyr;
R¹² is an amino acid side chain of 5FW or 1Nal;
R¹³ is an amino acid side chain of Phe or [(2S,3S)beta-Me-Phe]; and
R^{14A} is

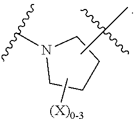

In another embodiment of the above formulae, P¹ is

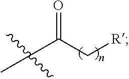

n is an integer from 1 to 6;
the C-terminus is selected from OH, N(R″)₂,

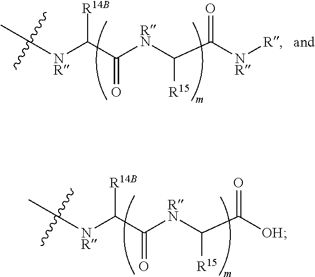

R′ is H;
R¹ is selected from the group consisting of an amino acid side chain of Ala, Cha, dAla, hCba, hCha, hLeu, HomoArg, HomoGlu, hTba, Leu, Met, and Nle;
R² is selected from the group consisting of an amino acid side chain of 3CIY, 3FY, 4COOHPhe, 4FPhe, 4PyA, 7AzaW, Ala, alloThr, aMeNle, aMeTyr, Cba, Chg, dAla, FSY, HomoArg, HomoGlu, hTyr, hyVal, Nle, NMeNle, OMeTyr, Tba, ThpA, ThpG, Thr, and Tyr;
R⁴ is selected from the group consisting of an amino acid side chain of Abu, allolle, aMeVal, alloThr, betaMelle, ChG, Cpg, dAla, HomoGlu, hyVal, Ile, Nle, NMeNle, NMeVal, Nva, PipAla, Tbg, tBuAla, Thr, and Val;
R⁵ is:
(i) selected from the group consisting of an amino acid side chain of Ala, dAla, Gly, HomoGlu, N(CH₂)₂COOHGly, NetGly, Ser, and Sar; or
(ii) selected from the group consisting of:

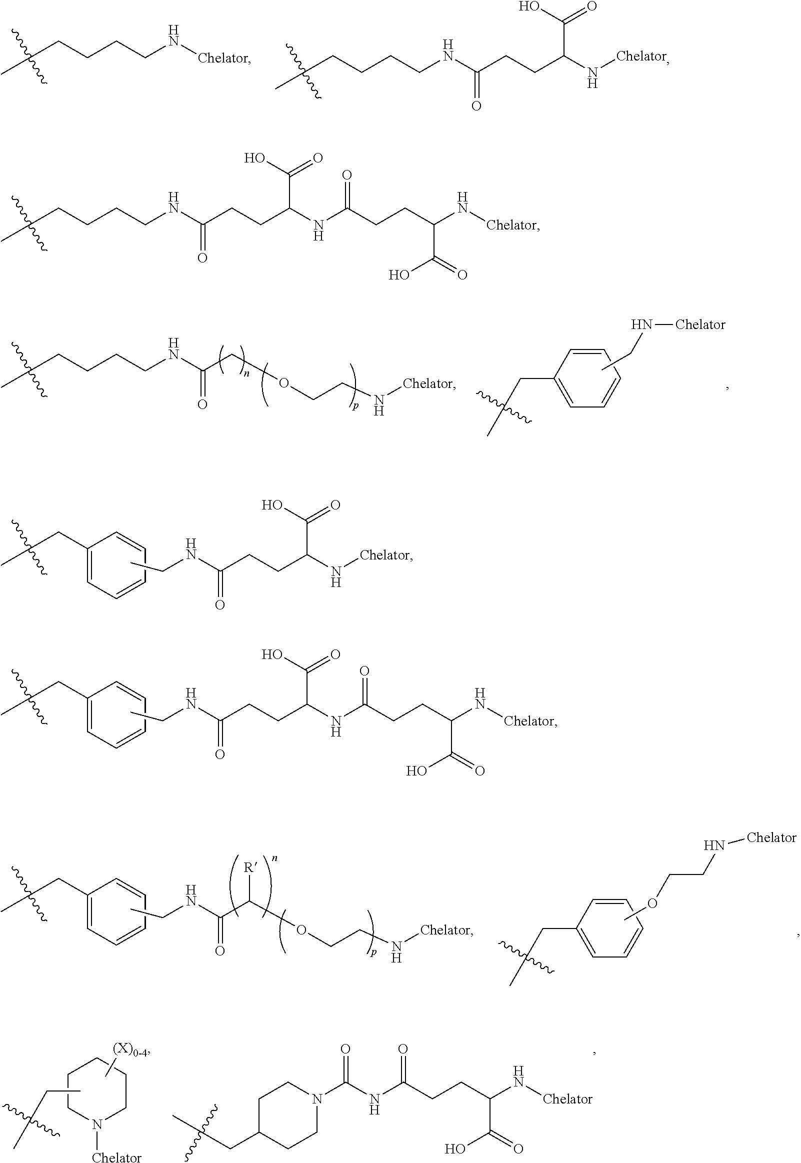

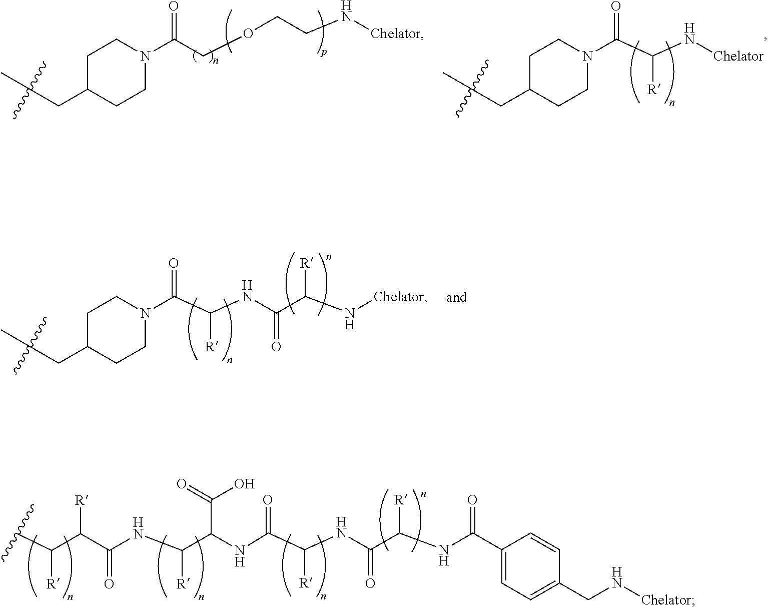

R⁶ is:
(i) selected from the group consisting of an amino acid side chain of Ala, aMeLys, Arg, Aze, Cit, Dab, dAla, Dap, dPro, HomoGlu, hSer, Lys, Lys(Ac), Lys(Me)2, Lys(Me)3, Nle, NMeLys, Orn, Pip, Pip(Ac)Ala, Pip(C4diacid)Ala, Pip(CH2COOH)Ala, Pip(CONH2)Ala, Pip(Me)Ala, PipAla, Pro, ThpA, and 4PyA; or
(ii) selected from the group consisting of:

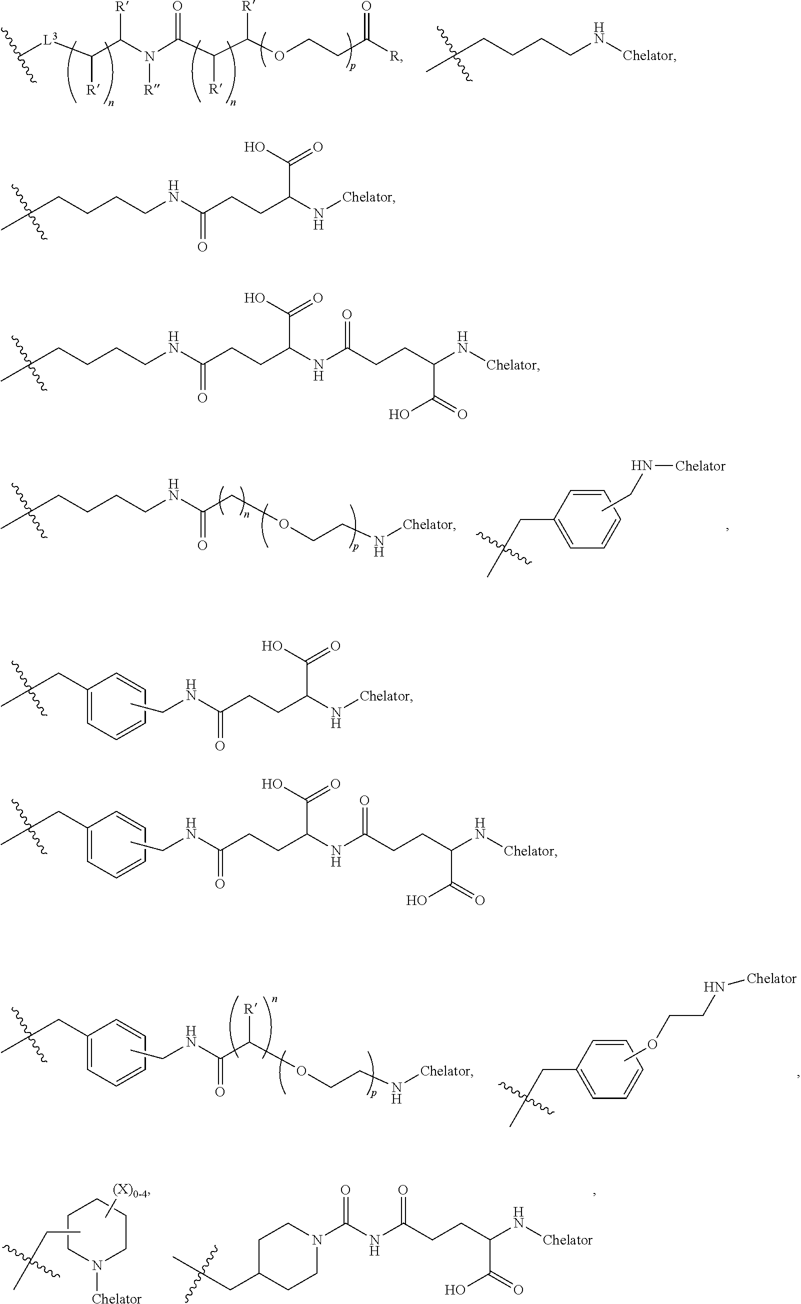

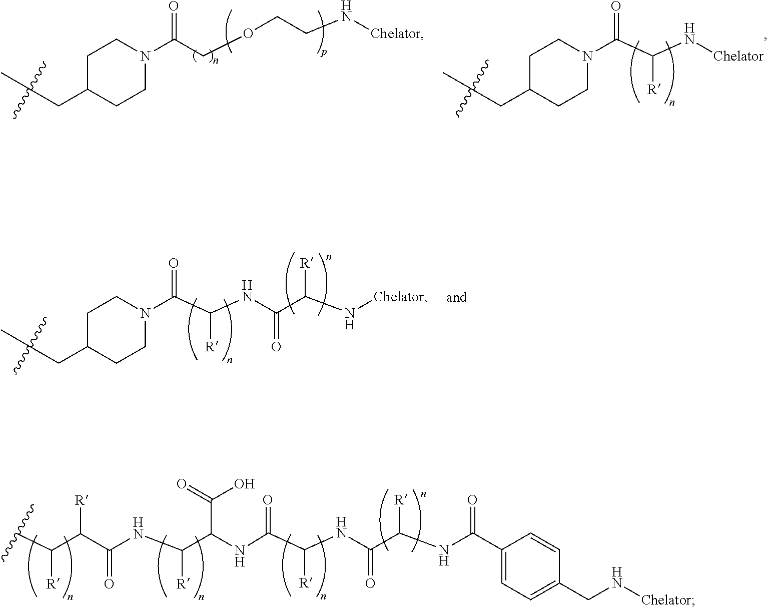

R⁷ is:
(i) selected from the group consisting of an amino acid side chain of (N-Me-ThpA), 4COOHPhe, 4OHCha, 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, and Tyr; or
(ii) selected from the group consisting of:

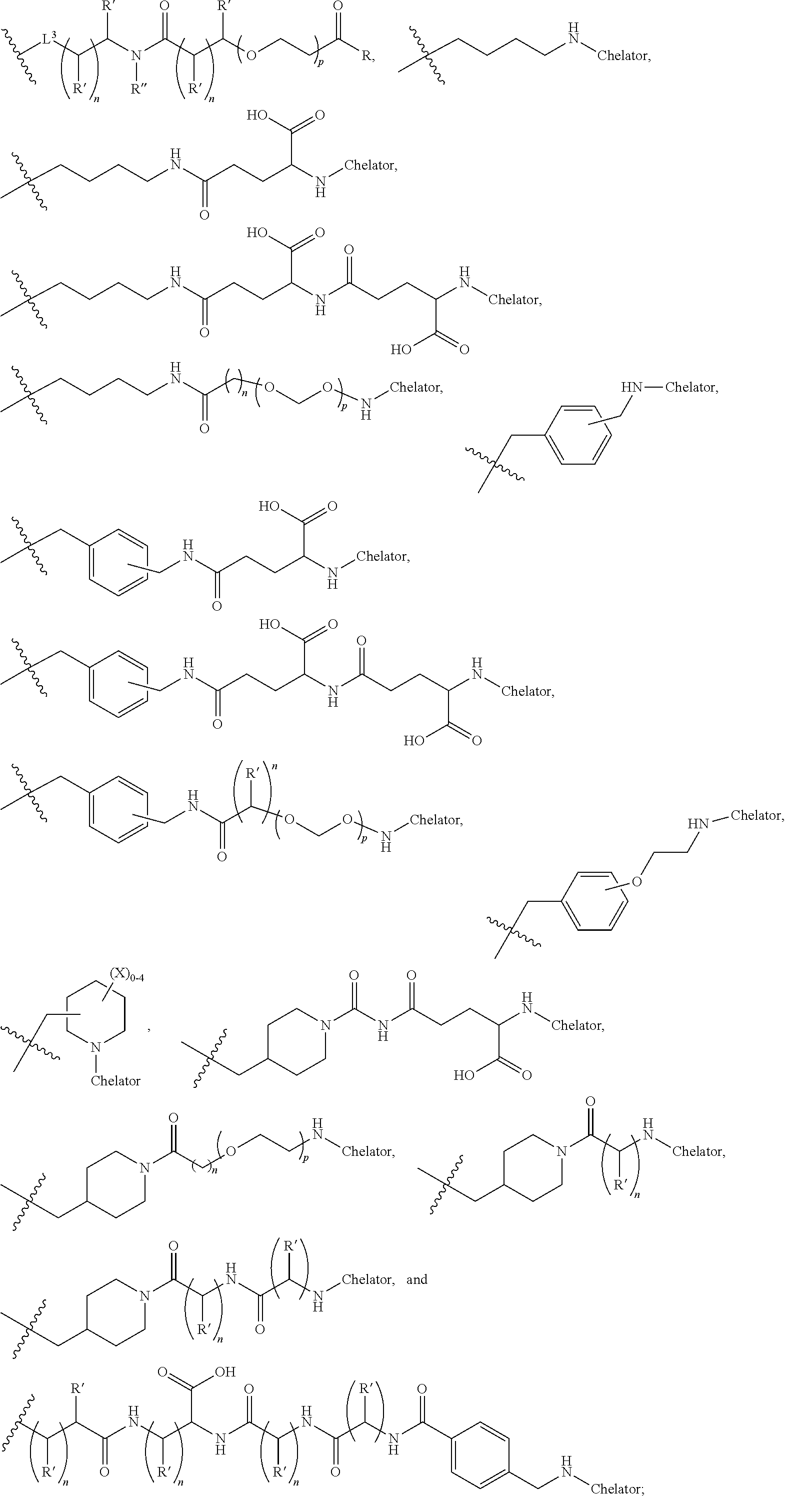

R⁸ is selected from the group consisting of an amino acid side chain of Ala, CysAcid, Asp, Dab, dAla, FSY, HomoGlu, Asn, and Ser;
R⁹ is:
(i) selected from the group consisting of an amino acid side chain of Abu, Aib, Ala, allolle, alloThr, aMeSer, aMeVal, ChG, Cpg, dAla, HomoGlu, hSer, hydroVal, Nle, NmeVal, Nva, Ser, Tbg, Thr, and Val; or
(ii) selected from the group consisting of:

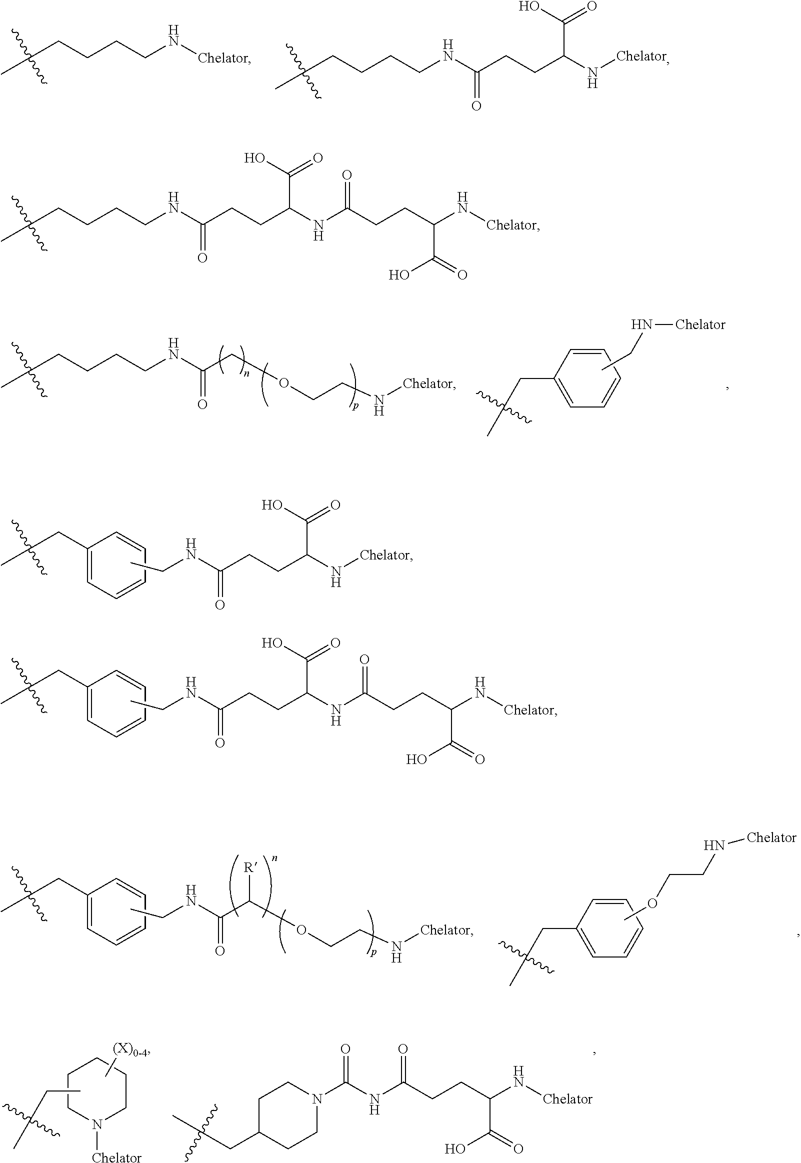

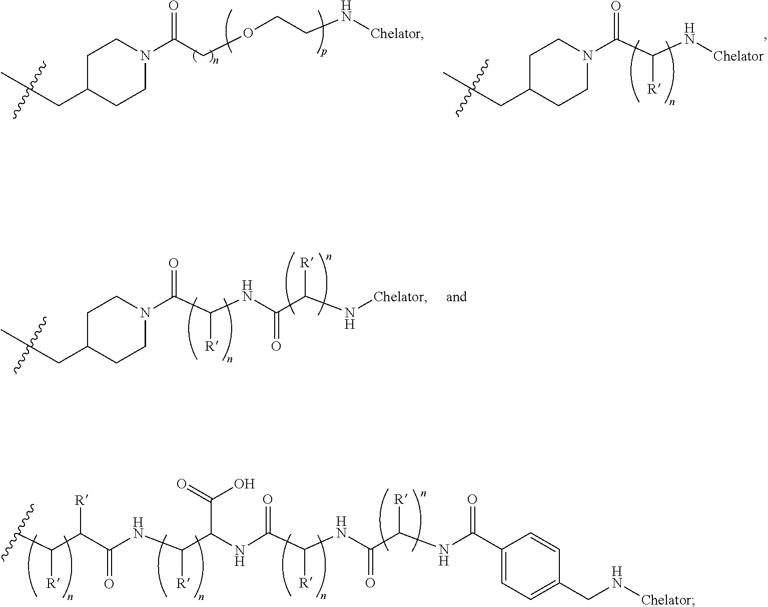

R¹⁰ is:
(i) selected from the group consisting of an amino acid side chain of (4-carbamoyl-Phe), 2OHPhe, 3FY, 3OHPhe, 3PyA, 4FPhe, Ala, alloThr, aMeLeu, aMeTyr, Aph(Cbm), Cha, Chg, CyanoButric, dAla, FSY, HomoArg, HomoGlu, hyVal, Nle, NMeTyr, OMeTyr, Tba, ThpA, Thr, and Tyr; or
(ii) selected from the group consisting of:

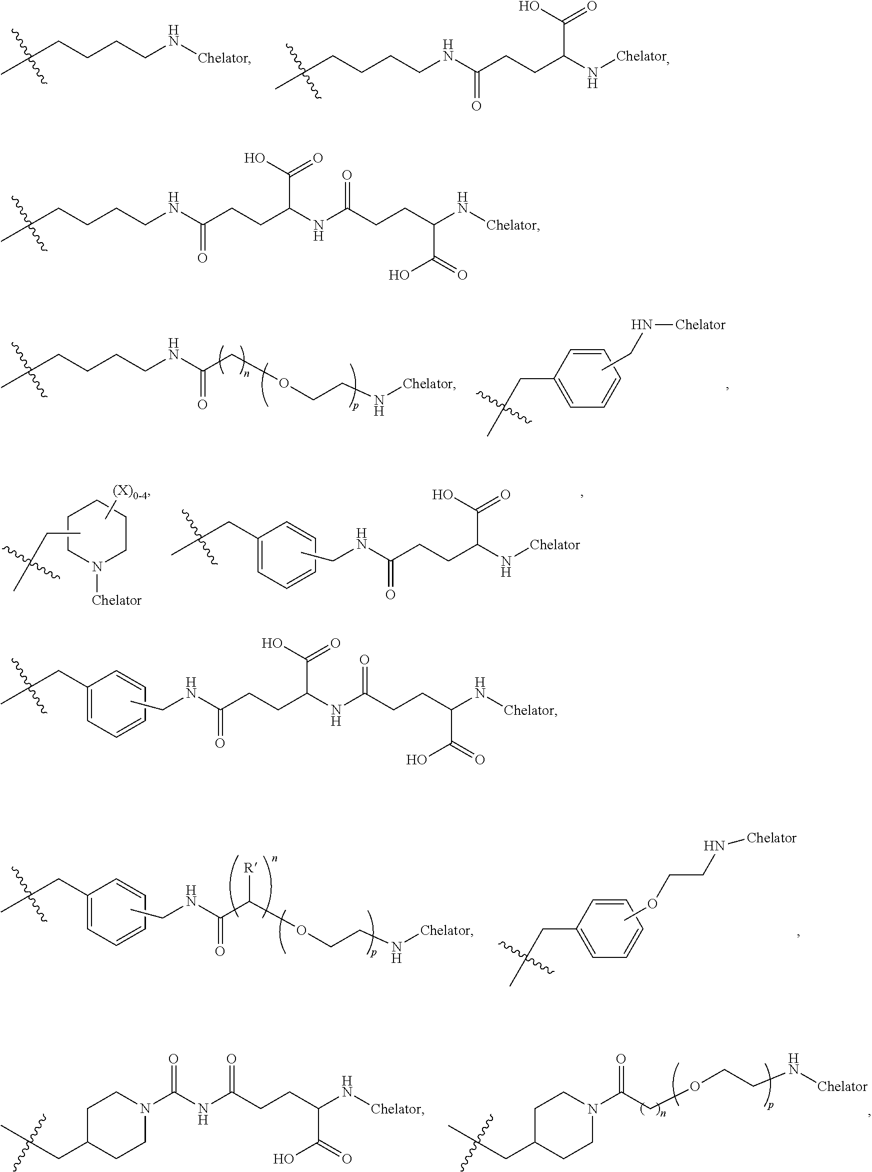

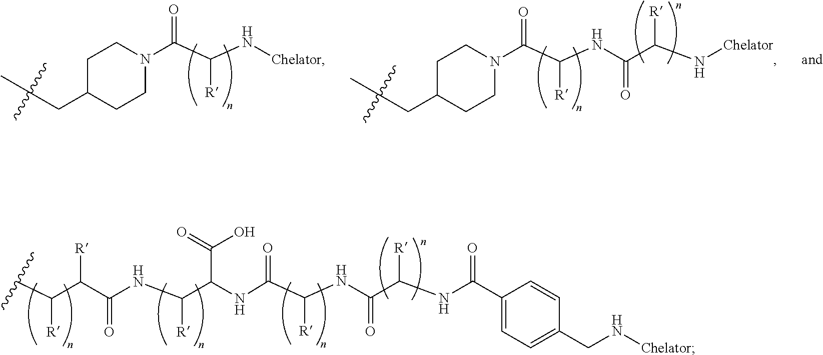

R¹² is selected from the group consisting of an amino acid side chain of (2S,3S-betaMe-5FW), 1Nal, 2MeW, 2Nal, 4CIW, 4FW, 4MeOW, 4MeW, 5CIW, 5FW, 5MeOW, 5MeW, 7NW, Ala, aMeW, dAla, hF, HomoArg, HomoGlu, Trp(CH₂COOH), Trp(Me), and Trp;
R¹³ is selected from the group consisting of an amino acid side chain of [(2S, 3R)beta-Me-Phe], [(2S, 3R)beta-OH-Phe], [(2S, 3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, ChA, dAla, Phe, His, HomoArg, HomoGlu, HomoPhe, Leu, Nle, NMeNle, NMePhe, Tbg, and tBuAla;
R^{14B} is selected from the group consisting of an amino acid side chain of 4PipAla, alloThr, aMeSer, Dab, Dap, dGlu, dLys, dThr, Glu, gE, hydroxyVal, Lys, NMeSer, Orn, Ser, and Thr;
R¹⁵ is selected from the group consisting of an amino acid side chain of dAla, dLys, dSer, dGlu, Gly, and betaAla;
B¹ is CH₂ or C(CH₃)₂;
C¹ is CH₂ or C(CH₃)₂; and
A¹ is

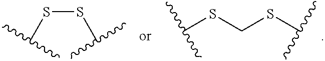

In still another embodiment of the above formulae, R¹ is selected from the group consisting of an amino acid side chain of Ala, Cha, dAla, hCba, hCha, hLeu, HomoArg, HomoGlu, hTba, Leu, Met, and Nle;
R² is selected from the group consisting of an amino acid side chain of 3CIY, 3FY, 4COOHPhe, 4FPhe, 4PyA, 7AzaW, Ala, alloThr, aMeNle, aMeTyr, Cba, Chg, dAla, FSY, HomoArg, HomoGlu, hTyr, hyVal, Nle, NMeNle, OMeTyr, Tba, ThpA, ThpG, Thr, and Tyr;
R⁴ is selected from the group consisting of an amino acid side chain ofAbu, allolle, aMeVal, alloThr, betaMelle, ChG, Cpg, dAla, HomoGlu, hyVal, Ile, Nle, NMeNle, NMeVal, Nva, PipAla, Tbg, tBuAla, Thr, and Val;
R⁵ is:
(i) selected from the group consisting of an amino acid side chain of Ala, dAla, Gly, HomoGlu, N(CH₂)₂COOHGly, NetGly, Ser, and Sar; or
(ii) selected from the group consisting of:

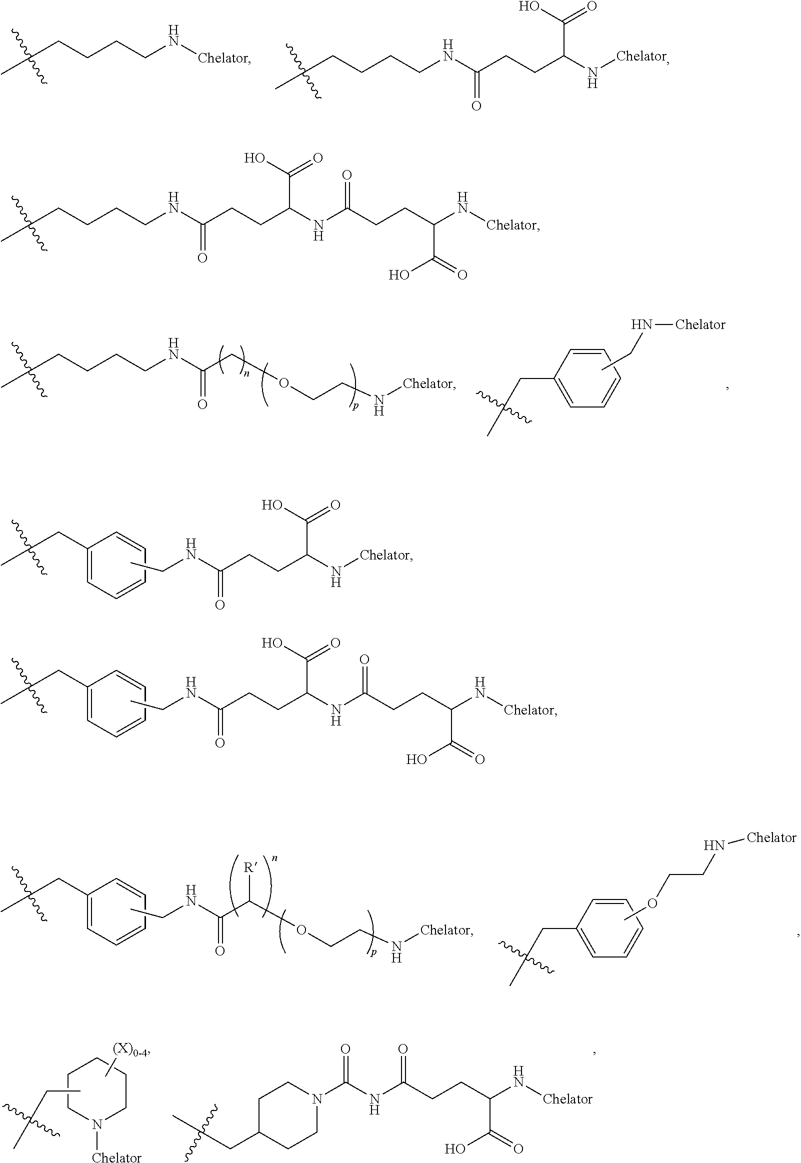

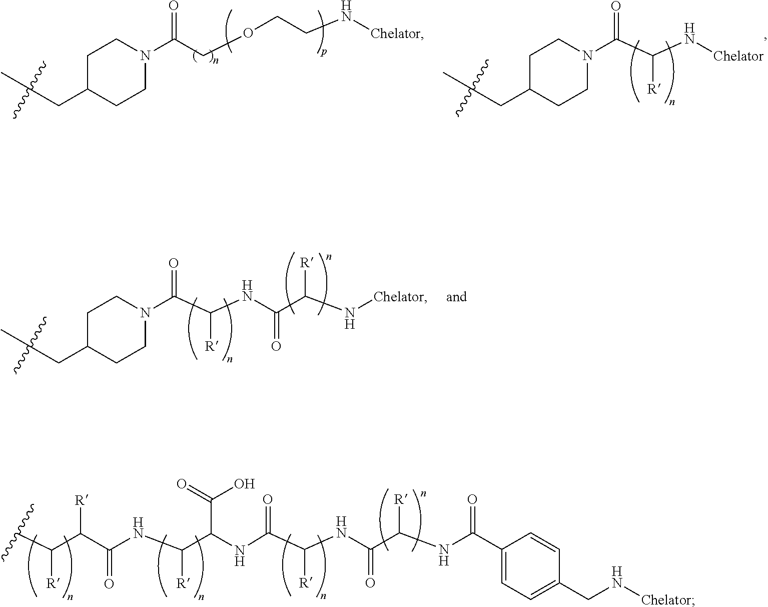

R⁶ is:
(i) selected from the group consisting of an amino acid side chain of Ala, aMeLys, Arg, Aze, Cit, Dab, dAla, Dap, dPro, HomoGlu, hSer, Lys, Lys(Ac), Lys(Me)2, Lys(Me)3, Nle, NMeLys, Orn, Pip, Pip(Ac)Ala, Pip(C4diacid)Ala, Pip(CH2COOH)Ala, Pip(CONH2)Ala, Pip(Me)Ala, PipAla, Pro, ThpA, and 4PyA;
(ii) selected from the group consisting of:

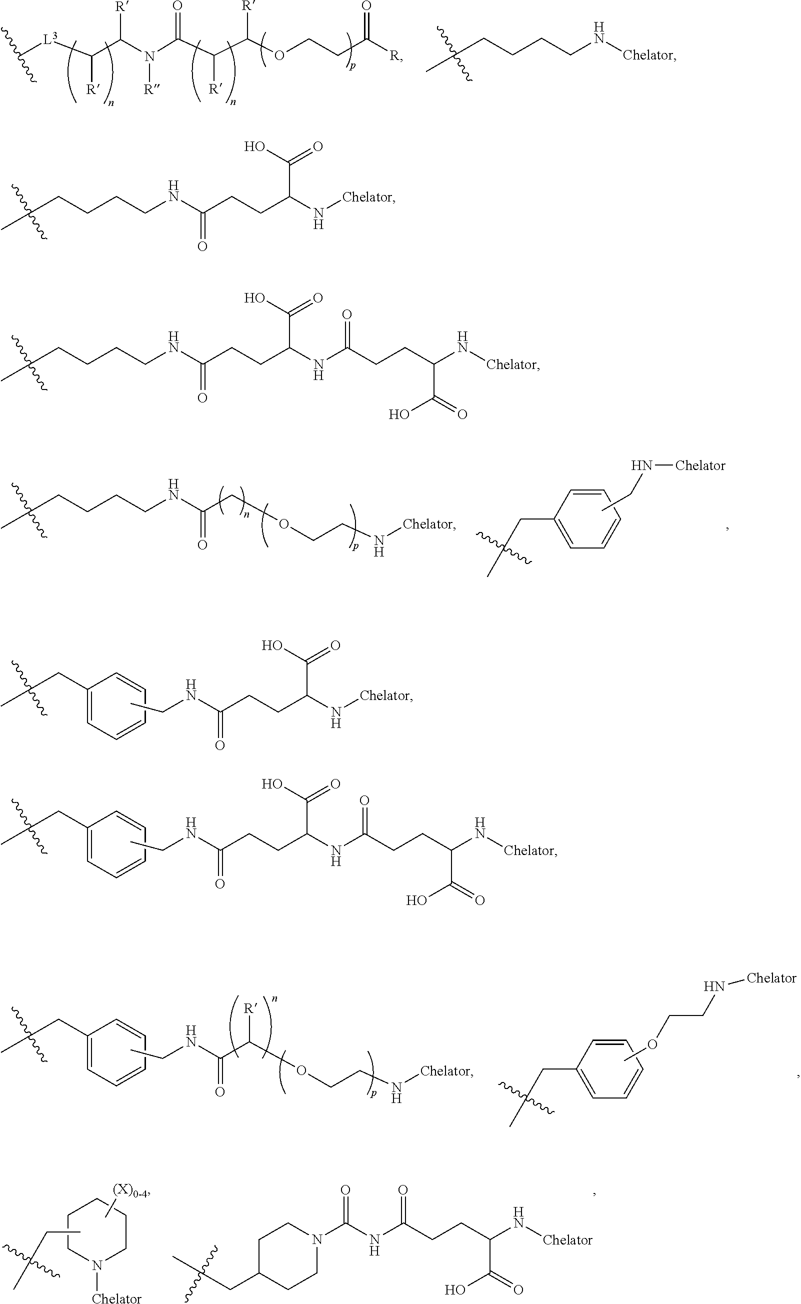

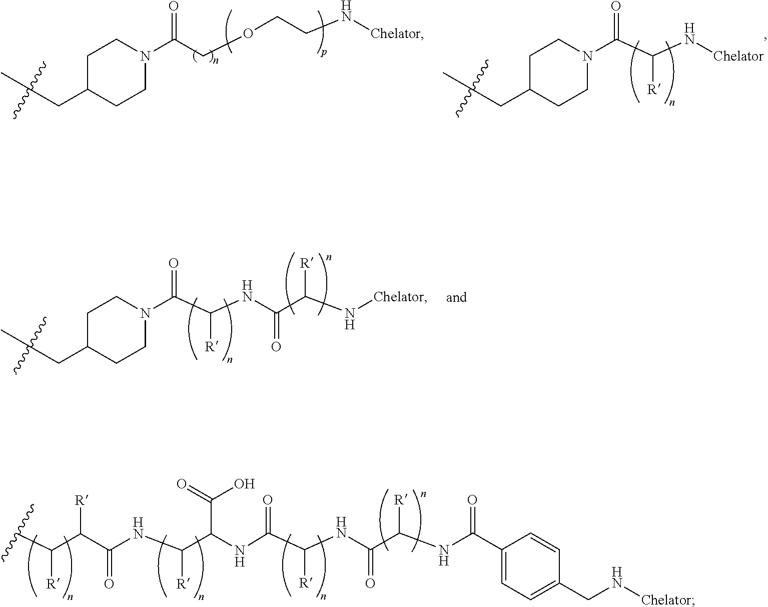

R⁷ is:
(i) selected from the group consisting of an amino acid side chain of (N-Me-ThpA), 4COOHPhe, 4OHCha, 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, and Tyr; or
(ii) selected from the group consisting of:

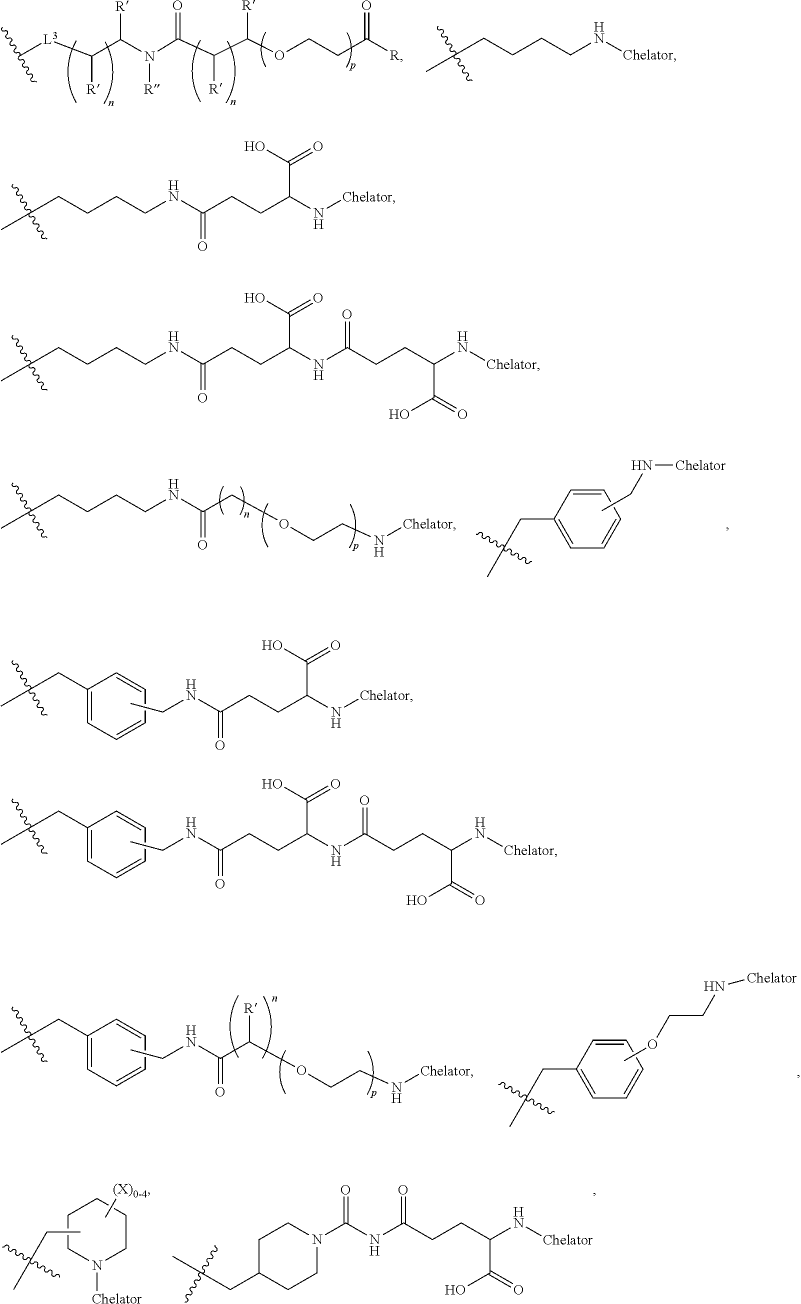

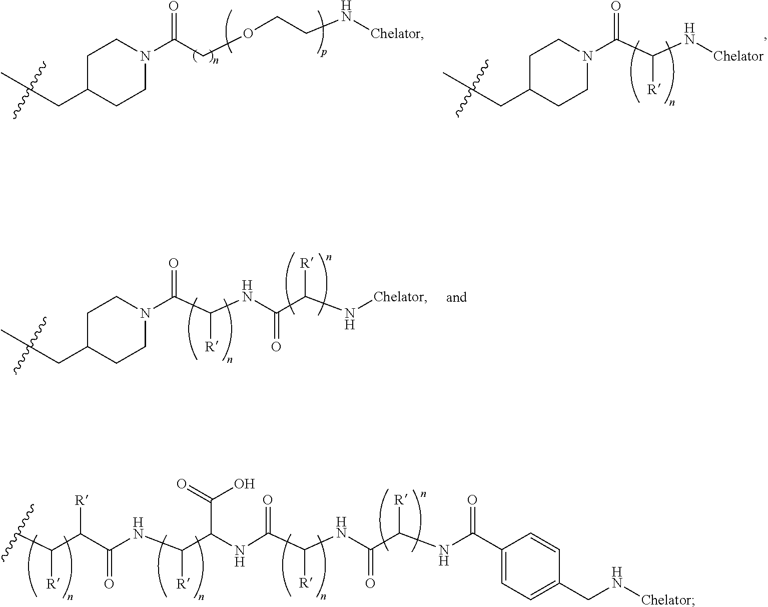

R⁸ is the group consisting of an amino acid side chain of Ala, CysAcid, Asp, Dab, dAla, FSY, HomoGlu, Asn, and Ser;
R⁹ is:
(i) selected from the group consisting of an amino acid side chain of Abu, Aib, Ala, allolle, alloThr, aMeSer, aMeVal, ChG, Cpg, dAla, HomoGlu, hSer, hydroVal, Nle, NmeVal, Nva, Ser, Tbg, Thr, and Val; or
(ii) selected from the group consisting of:

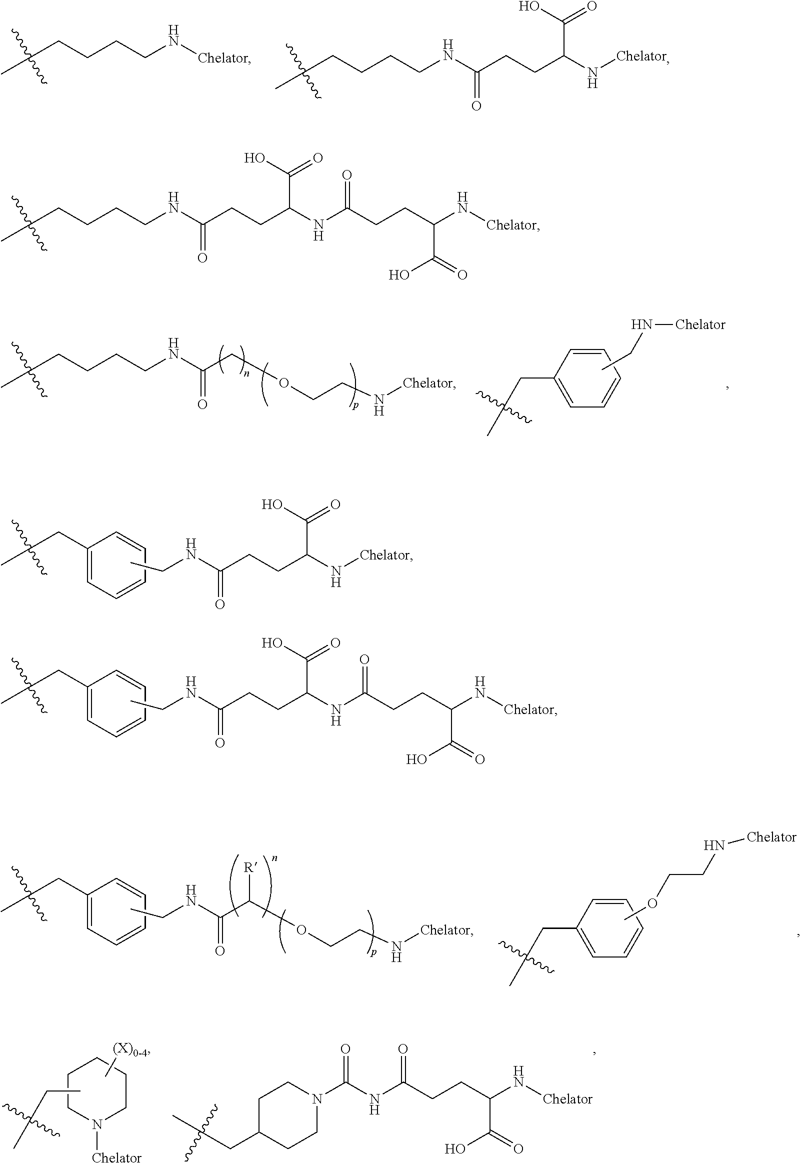

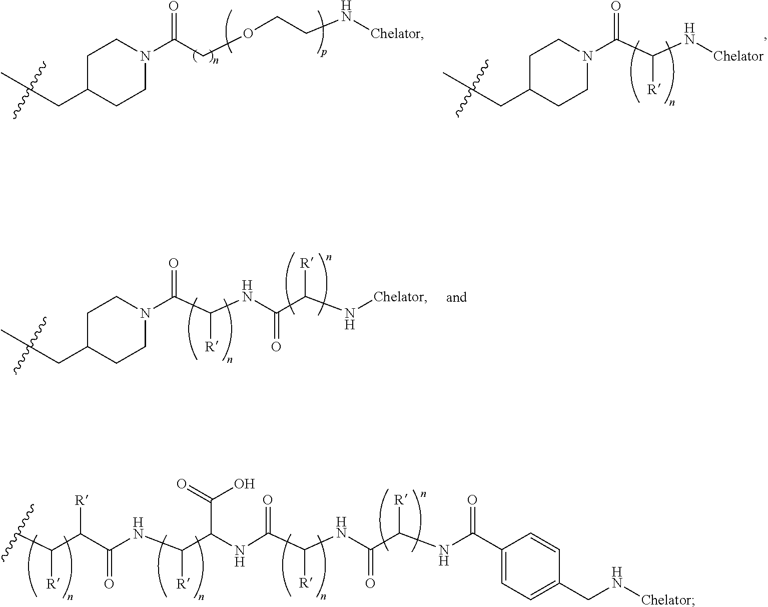

R¹⁰ is:
(i) selected from the group consisting of an amino acid side chain of (4-carbamoyl-Phe), 2OHPhe, 3FY, 3OHPhe, 3PyA, 4FPhe, Ala, alloThr, aMeLeu, aMeTyr, Aph(Cbm), Cha, Chg, CyanoButric, dAla, FSY, HomoArg, HomoGlu, hyVal, Nle, NMeTyr, OMeTyr, Tba, ThpA, Thr, and Tyr; or
(ii) selected from the group consisting of:

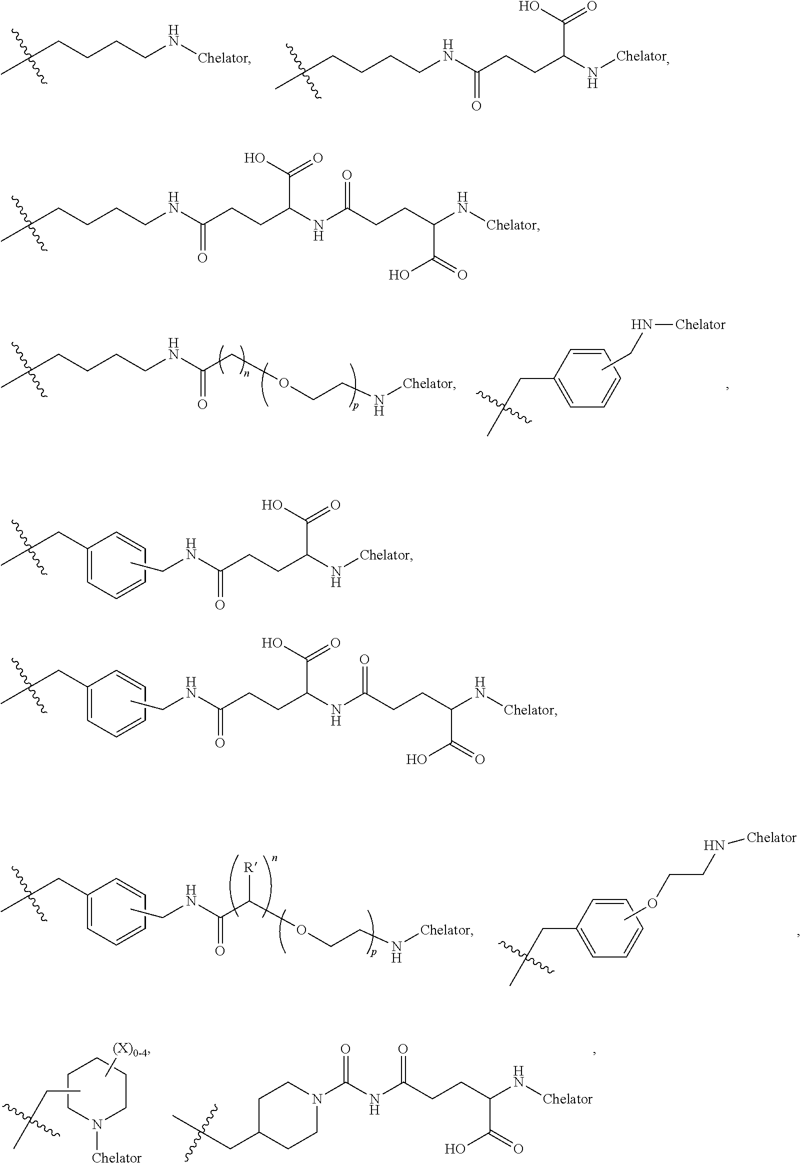

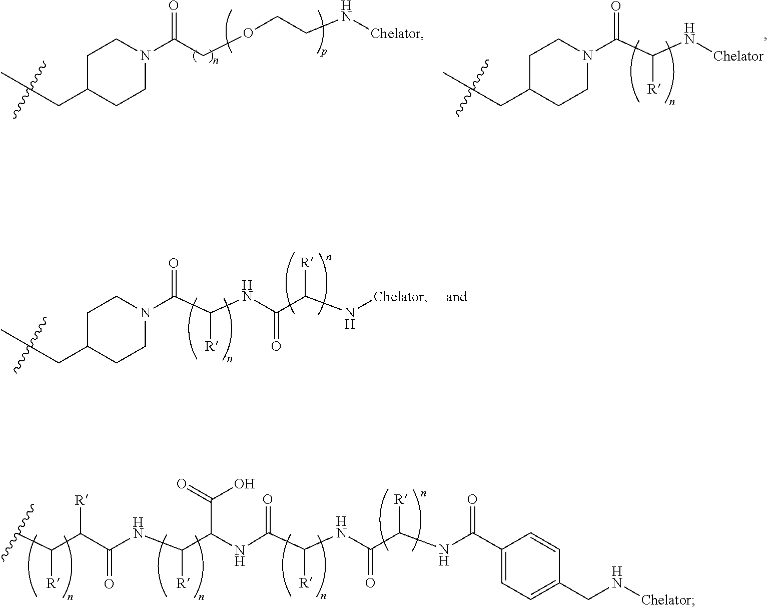

R¹² is selected from the group consisting of an amino acid side chain of (2S,3S-betaMe-5FW), 1Nal, 2MeW, 2Nal, 4CIW, 4FW, 4MeOW, 4MeW, 5CIW, 5FW, 5MeOW, 5MeW 7NW, Ala, aMeW, dAla, hF, HomoArg, HomoGlu, Trp(CH₂COOH), Trp(Me), and Trp;
R¹³ is selected from the group consisting of an amino acid side chain of [(2S, 3R)beta-Me-Phe], [(2S, 3R)beta-OH-Phe], [(2S, 3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, ChA, dAla, Phe, His, HomoArg, HomoGlu, HomoPhe, Leu, Nle, NMeNle, NMePhe, Tbg, and tBuAla;
R^{14B} is selected from the group consisting of an amino acid side chain of 4PipAla, alloThr, aMeSer, Dab, Dap, dGlu, dLys, dThr, Glu, gE, hydroxyVal, Lys, NMeSer, Orn, Ser, and Thr; and
R¹⁵ is selected from the group consisting of an amino acid side chain of dAla, dLys, dSer, dGlu, Gly, and betaAla.

In an embodiment of the above formulae, R¹ is selected from the group consisting of an amino acid side chain of hLeu, hCha, hCba, and or Nle;
R² is an amino acid side chain of Tyr;
R⁴ is selected from the group consisting of an amino acid side chain of Ile, Tbg, and Val;
R⁵ is an amino acid side chain of Gly or Sar;
R⁶ is:
(i) selected from the group consisting of an amino acid side chain of PipAla, Lys, ThpA, Pip(CH2COOH)Ala, Cit, Lys(Ac), Pip(Ac)Ala, Pip(CONH2)Ala, Pip(Me)Ala, Lys(Me)2, and Lys(Me)3; or
(ii) selected from the group consisting of:

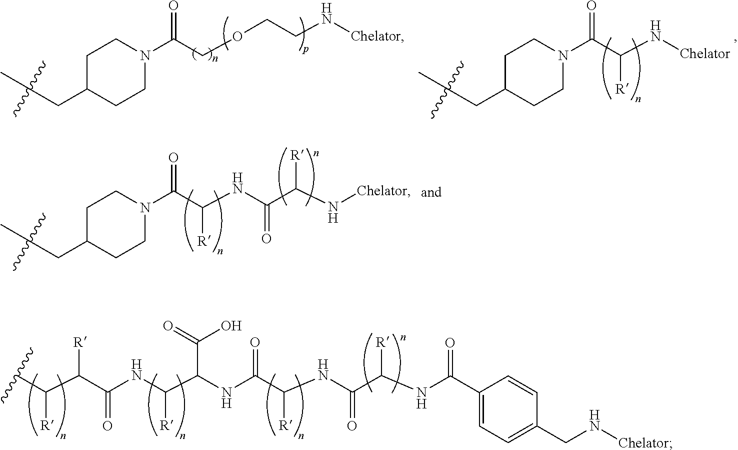

R⁷ is:
(i) selected from the group consisting of an amino acid side chain of 4COOHPhe, 4Pya, PipAla, and ThpA; or
(ii) selected from the group consisting of:

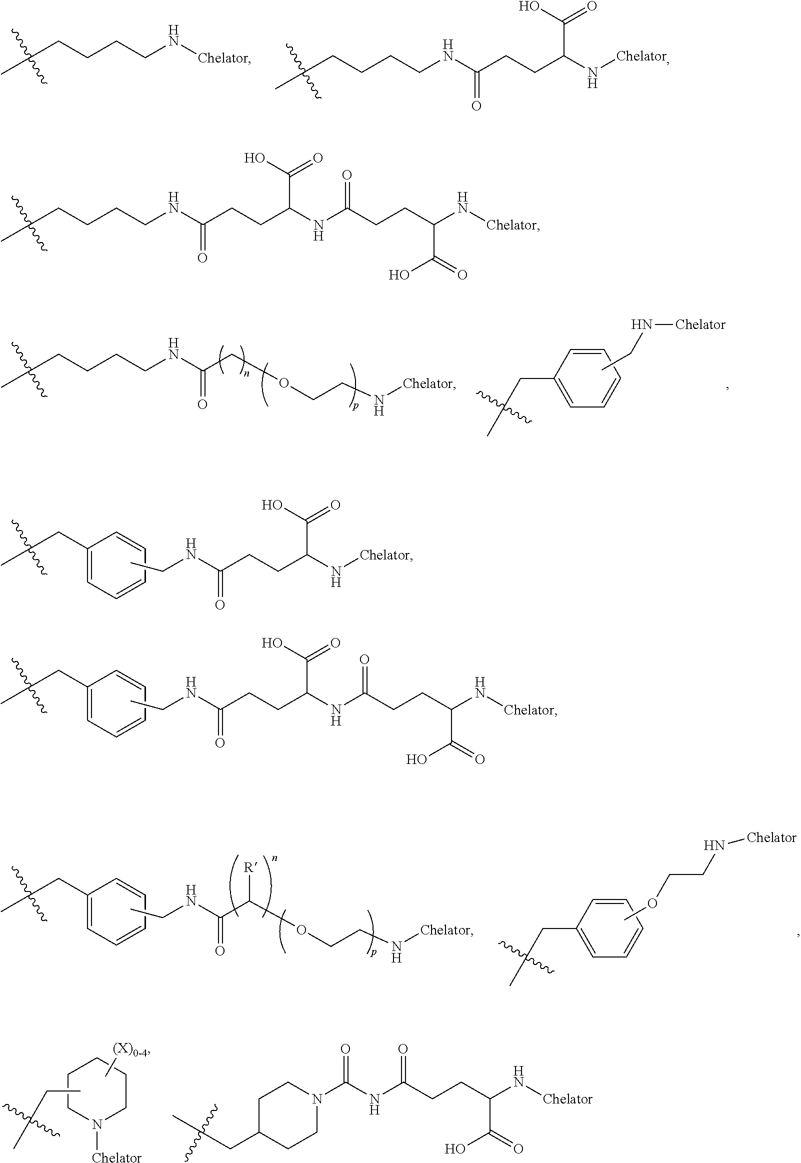

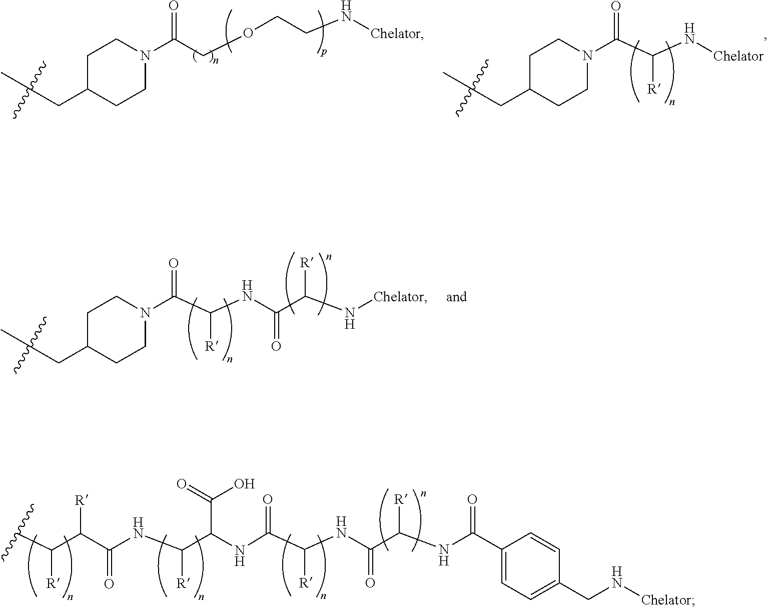

R⁸ is an amino acid side chain of Asp or CysAcid;
R⁹ is an amino acid side chain of Val;
R¹⁰ is an amino acid side chain of Tyr;
R¹² is an amino acid side chain of 5FW or 1Nal;
R¹³ is an amino acid side chain of Phe or [(2S, 3S)beta-Me-Phe];
R^{14B} is an amino acid side chain of hydroxyVal or Thr; and
R¹⁵ is selected from the group consisting of an amino acid side chain of dAla, dSer, dGlu, Gly, and betaAla.

In another embodiment of the above formulae, P¹ is

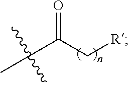

n is an integer from 1 to 6;
the C-terminus is selected from OH, N(R″)₂,

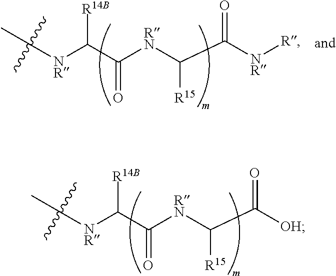

R′ is H;
R¹ is an amino acid side chain of hLeu or hCha;
R² is an amino acid side chain of Tyr;
R⁴ is an amino acid side chain of Val or Tbg;
R⁵ is an amino acid side chain of Gly or Sar;
R⁶ is an amino acid side chain of Pip(CH2COOH)Ala or Pip(PEG4-DOTA)Ala;
R⁷ is an amino acid side chain of Pip(PEG4-DOTA)Ala or ThpA;
R⁸ is an amino acid side chain of Asp or CysAcid;
R⁹ is an amino acid side chain of Val or Lys(DOTA);
R¹⁰ is an amino acid side chain of Tyr or Aph(Cbm).
R¹² is an amino acid side chain of 5FW or 1Nal;
R¹³ is an amino acid side chain of Phe or [(2S, 3S)beta-Me-Phe];
R^{14B} is an amino acid side chain of hydroxyVal or Thr;
R¹⁵ is selected from the group consisting of an amino acid side chain of dAla, dSer, and dGlu;
B¹ is CH₂ or C(CH₃)₂;
C¹ is CH₂ or C(CH₃)₂; and
A¹ is

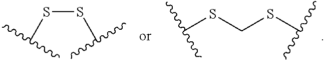

In another embodiment of the above formulae, A¹ is

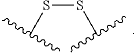 [SMILES: *SS*]

In an embodiment of the above formulae, R¹ is selected from the group consisting of an amino acid side chain of Ala, Cha, dAla, hCba, hCha, hLeu, HomoArg, HomoGlu, hTba, Leu, Met, and Nle. In another embodiment, R¹ is selected from the group consisting of an amino acid side chain of hLeu, hCha, hCba and Nle. In yet another embodiment, R¹ is an amino acid side chain of hLeu or hCha.

In still another embodiment of the above formulae, R² is selected from the group consisting of an amino acid side chain of 3CIY, 3FY, 4COOHPhe, 4FPhe, 4PyA, 7AzaW, Ala, alloThr, aMeNle, aMeTyr, Cba, Chg, dAla, FSY, HomoArg, HomoGlu, hTyr, hyVal, Nle, NMeNle, OMeTyr, Tba, ThpA, ThpG, Thr, and Tyr. In an embodiment, an amino acid side chain of Tyr.

In another embodiment of the above formulae, R⁴ is selected from the group consisting of an amino acid side chain of Abu, allolle, aMeVal, alloThr, betaMelle, ChG, Cpg, dAla, HomoGlu, hyVal, Ile, Nle, NMeNle, NMeVal, Nva, PipAla, Tbg, tBuAla, Thr, and Val.

In another embodiment, R⁴ is selected from the group consisting of an amino acid side chain of Val, PipAla, Ile, Tbg, and NMelle. In yet another embodiment, R⁴ is the amino acid side chain of Val or Tbg. In still another embodiment, R⁴ is the amino acid side chain of Tbg.

In another embodiment of the above formulae, R⁵ is:
(i) selected from the group consisting of an amino acid side chain of Ala, dAla, Gly, HomoGlu, N(CH₂)₂COOHGly, NetGly, Ser, and Sar; or
(ii) selected from the group consisting of:

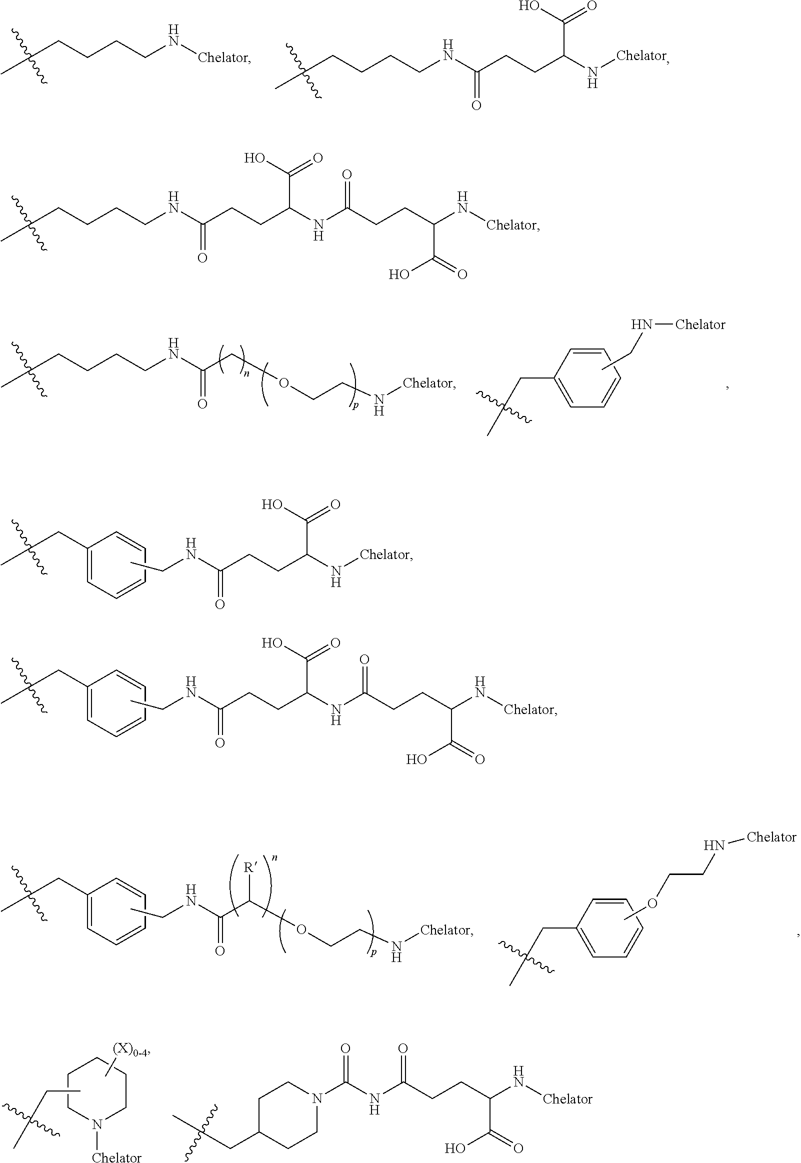

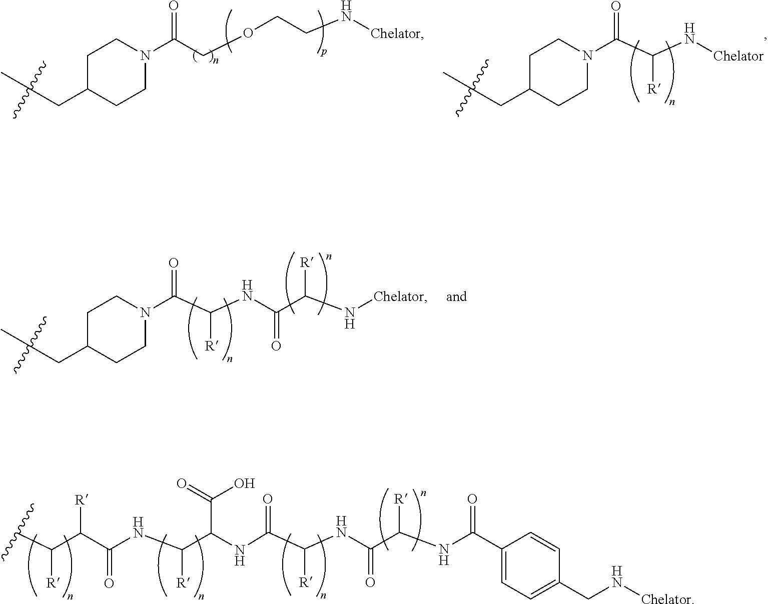

In yet another embodiment, R⁵ is an amino acid side chain of Gly or Sar. In still another embodiment, R⁵ is an amino acid side chain of Gly or dLys(DOTA). In an embodiment, R⁵ is the amino acid side chain of Gly. In another embodiment, R⁵ is an amino acid side chain of dLys(DOTA).

In still another embodiment of the above formulae, R⁶ is:
(i) selected from the group consisting of an amino acid side chain of Ala, aMeLys, Arg, Aze, Cit, Dab, dAla, Dap, dPro, HomoGlu, hSer, Lys, Lys(Ac), Lys(Me)2, Lys(Me)3, Nle, NMeLys, Orn, Pip, Pip(Ac)Ala, Pip(C4diacid)Ala, Pip(CH2COOH)Ala, Pip(CONH2)Ala, Pip(Me)Ala, PipAla, Pro, ThpA, and 4PyA; or
(ii) selected from the group consisting of:

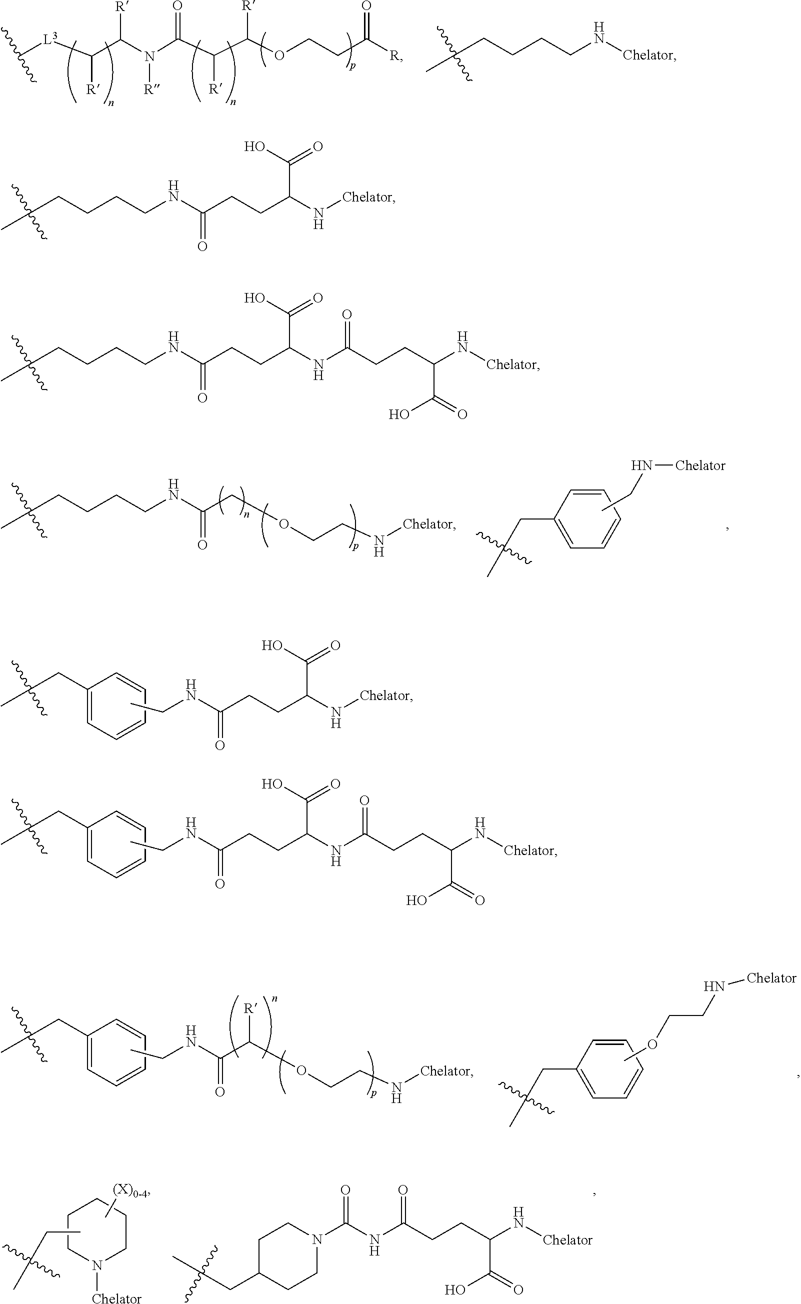

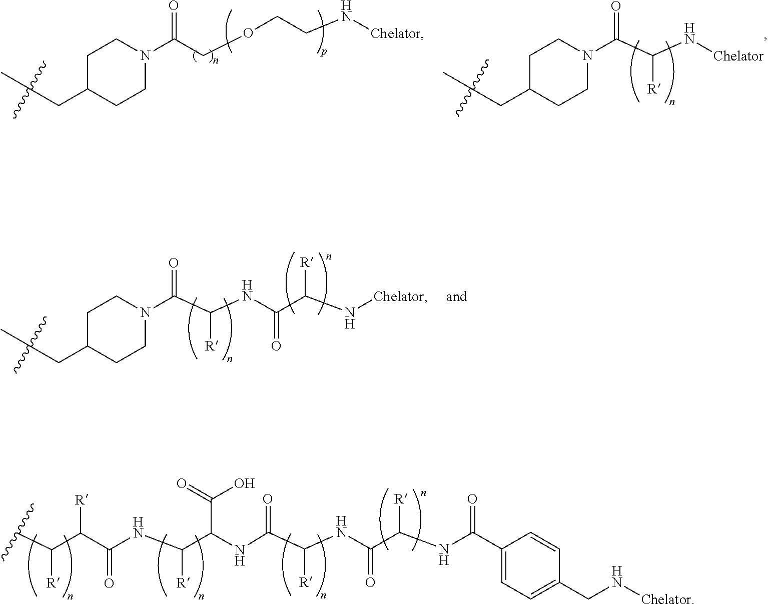

In another embodiment of the above formulae, R⁶ is selected from the group consisting of an amino acid side chain of Lys, Lys(DOTA), Lys(gE-DOTA), Lys(gE-gE-DOTA), Lys(PEG4-DOTA), PipAla, Pip(DOTA)Ala, Pip(PEG4-DOTA)Ala, Pip(PEG2-DOTA)Ala, Pip(gE-DOTA)Ala, Pip(R-DOTAGA)Ala, ThpA, Pip(gE-gE-DOTA)Ala, Cit, Pip(CH2COOH)Ala, Lys(Ac), Pip(Ac)Ala, Pip(CONH2)Ala, Glu[εLys(OH)-Val-Met-AmBz-DOTA], Pip(Me)Ala, Lys(Me)2, and Lys(Me)3. In yet another embodiment, R⁶ is selected from the group consisting of the amino acid side chain of Lys, Lys(DOTA), Pip(PEG4-DOTA)Ala, Pip(CH2COOH)Ala, and PipAla. In still another embodiment, R⁶ is selected from the group consisting of the amino acid side chain of Pip(CH2COOH)Ala, Lys, Lys(DOTA), or Pip(PEG4-DOTA)Ala. In an embodiment, R⁶ is the amino acid side chain of Pip(CH2COOH)Ala or Pip(PEG4-DOTA)Ala.

In another embodiment of the above formulae, R⁷ is:
(i) selected from the group consisting of an amino acid side chain of (N-Me-ThpA), 4COOHPhe, 4OHCha, 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, and Tyr; or
(ii) selected from the group consisting of:

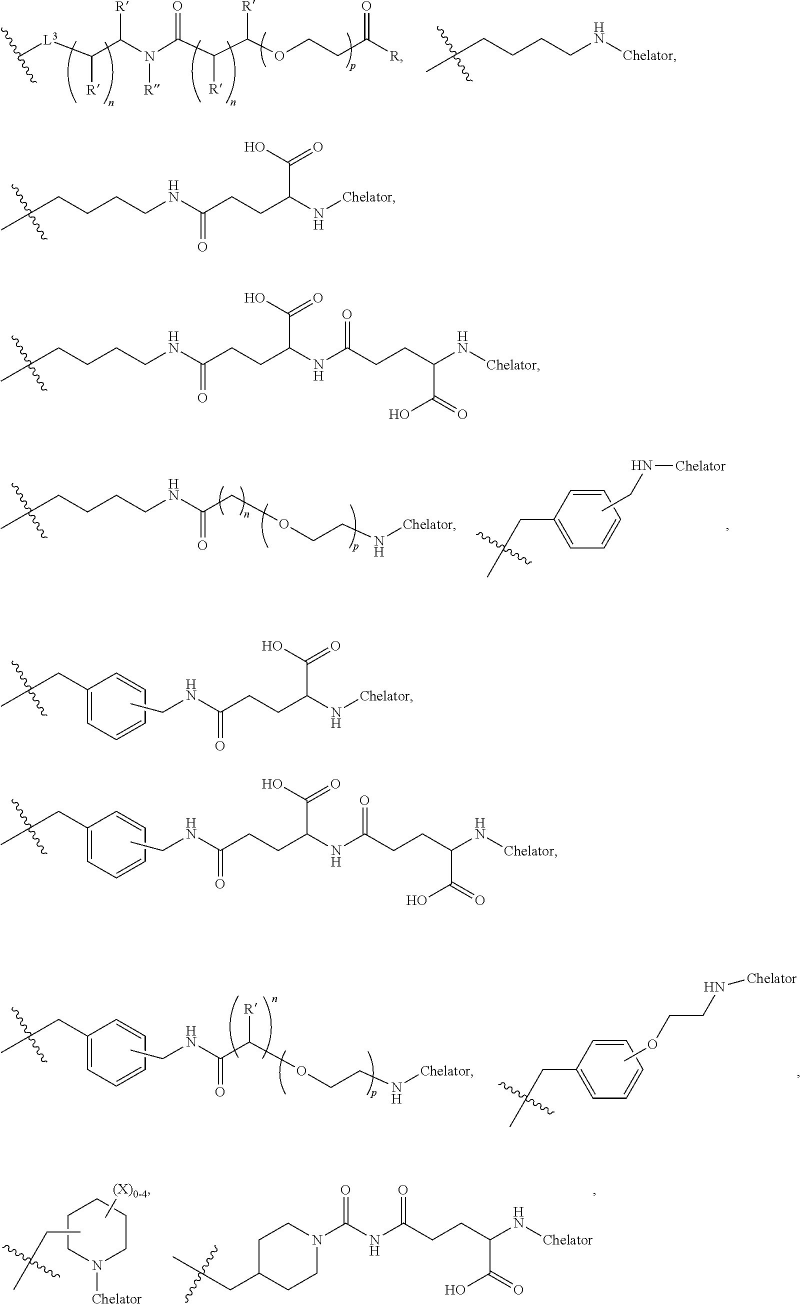

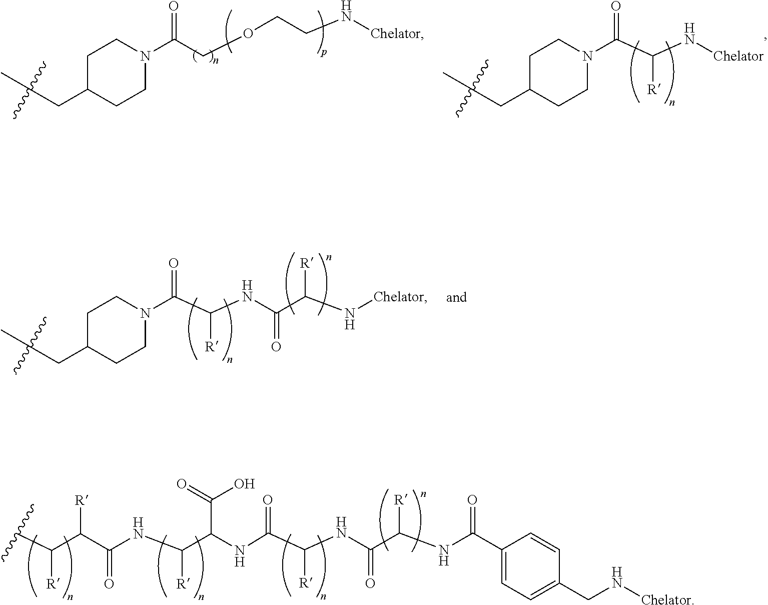

In an embodiment of the above formulae, R⁷ is selected from the group consisting of an amino acid side chain of Tyr, AEF(DOTA), Lys(DOTA), MAF(DOTA), CHA, PipAla, 4PyA, MAF(gE-DOTA), MAF(gE-gE-DOTA), MAF(PEG4-DOTA), MAF(gE-gE-gE-DOTA), ThpA, MAF(PEG8-DOTA), 4COOHPhe, Pip(CH2COOH)Ala, Pip(DOTA)Ala, Pip(PEG4-DOTA)Ala, 4OHCha, Pip(PEG2-DOTA)Ala, Pip(gE-DOTA)Ala, Pip(R-DOTAGA)Ala, Glu[εLys(OH)-Val-Met-AmBz-DOTA], Lys(PEG4-DPTA), and Pip(Glu-DOTA)Ala. In another embodiment, R⁷ is selected from the group consisting of the amino acid side chain of 4PyA, Pip(PEG4-DOTA)Ala, and ThpA. In yet another embodiment, R⁷ is the amino acid side chain of Pip(PEG4-DOTA)Ala or ThpA. In still another embodiment, R⁷ is an amino acid side chain of Pip(PEG4-DOTA)Ala. In an embodiment, R⁷ is an amino acid side chain of ThpA.

In yet another embodiment of the above formulae, R⁸ is the group consisting of an amino acid side chain of Ala, CysAcid, Asp, Dab, dAla, FSY, HomoGlu, Asn, and Ser. In still another embodiment, R⁸ is an amino acid side chain of Asp or CysAcid. In still another embodiment, R⁸ is the amino acid side chain of Asp. In yet another embodiment, R⁸ is an amino acid side chain of CysAcid.

In another embodiment of the above formulae, R⁹ is:
(i) selected from the group consisting of an amino acid side chain of Abu, Aib, Ala, allolle, alloThr, aMeSer, aMeVal, ChG, Cpg, dAla, HomoGlu, hSer, hydroVal, Nle, NMeVal, Nva, Ser, Tbg, Thr, and Val; or
(ii) selected from the group consisting of:

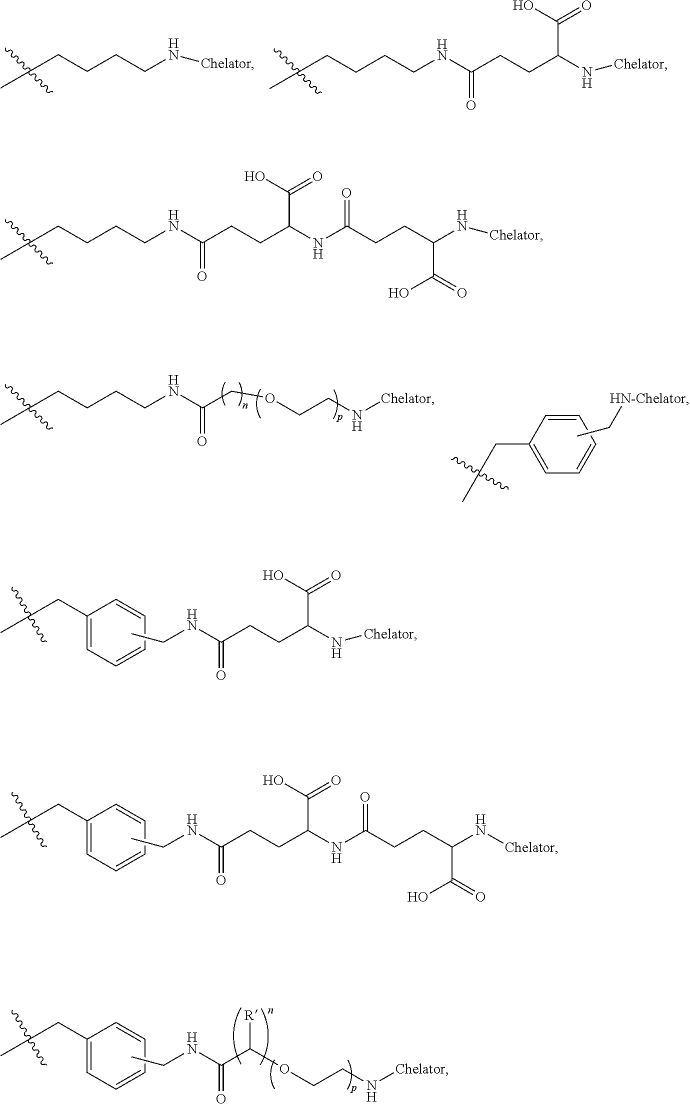

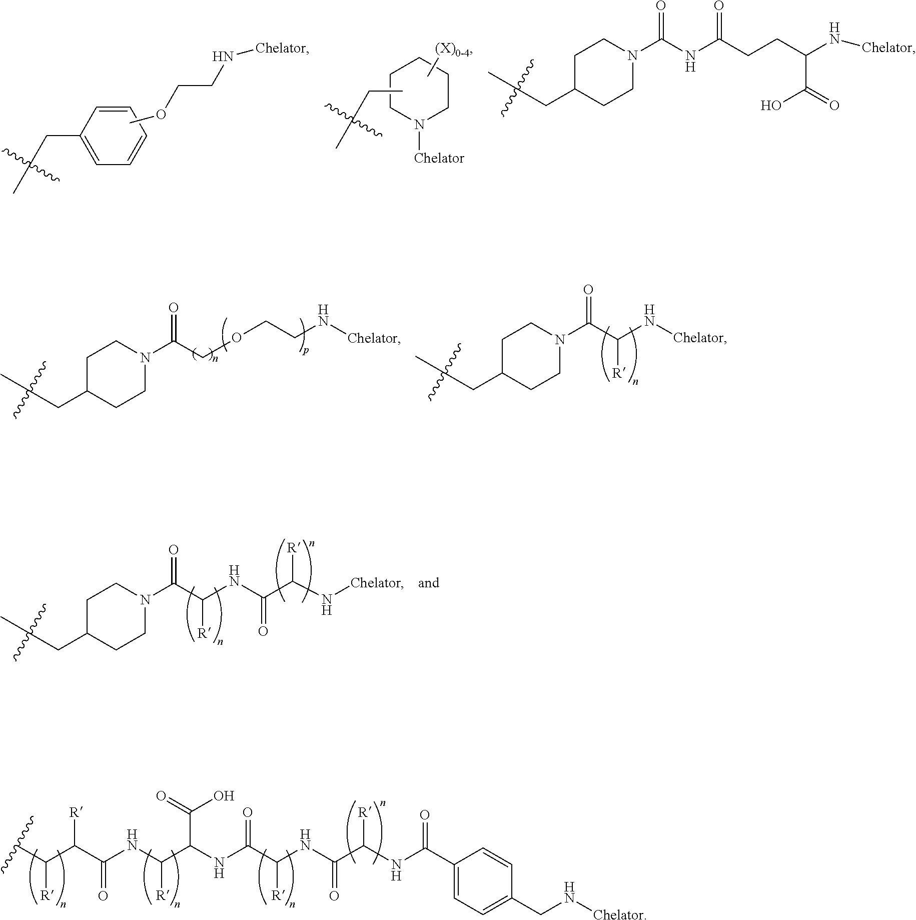

In an embodiment of the above formulae, R⁹ is an amino acid side chain of Val or Lys(DOTA). In another embodiment, R⁹ is the amino acid side chain of Val. In another embodiment, R⁹ is an amino acid side chain Lys(DOTA).

In an embodiment of the above formulae, R¹⁰ is:
(i) selected from the group consisting of an amino acid side chain of (4-carbamoyl-Phe), 2OHPhe, 3FY, 3OHPhe, 3PyA, 4FPhe, Ala, alloThr, aMeLeu, aMeTyr, Aph(Cbm), Cha, Chg, CyanoButric, dAla, FSY, HomoArg, HomoGlu, hyVal, Nle, NMeTyr, OMeTyr, Tba, ThpA, Thr, and Tyr; or
(ii) selected from the group consisting of:

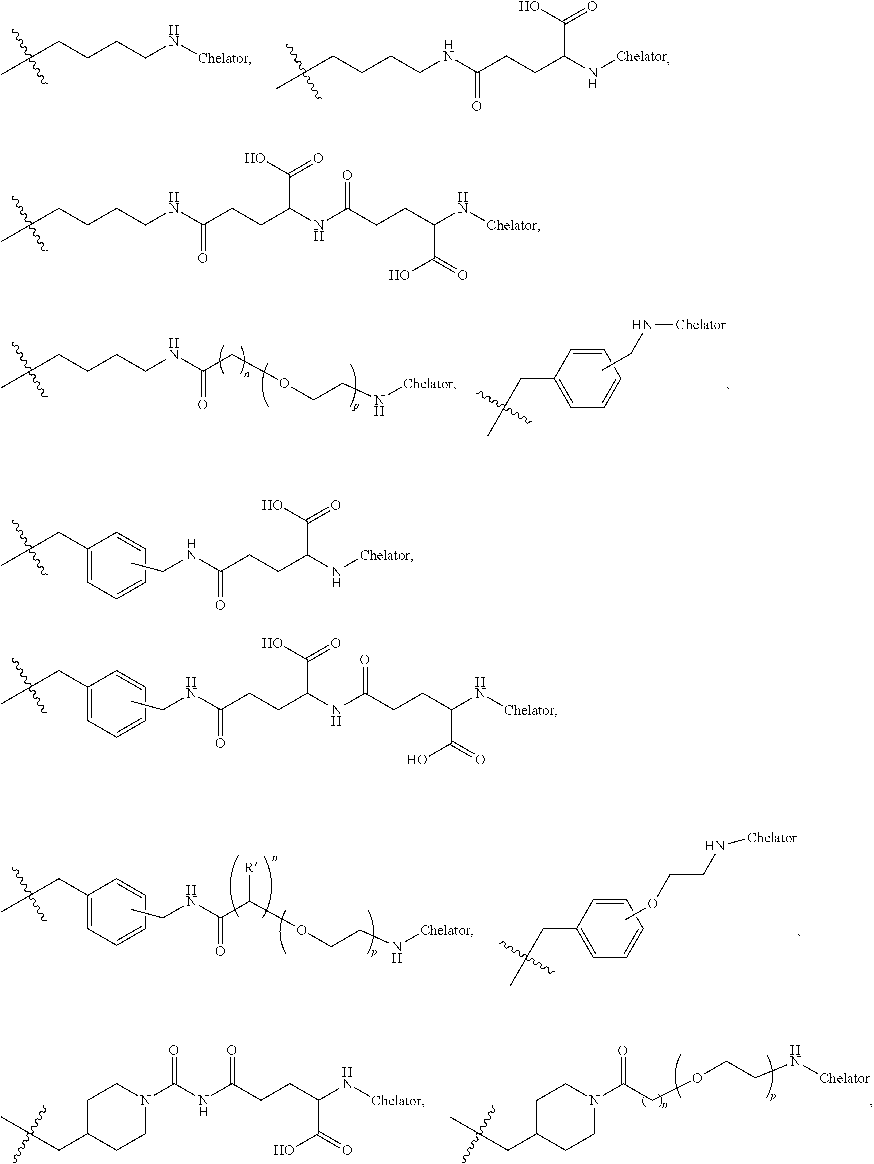

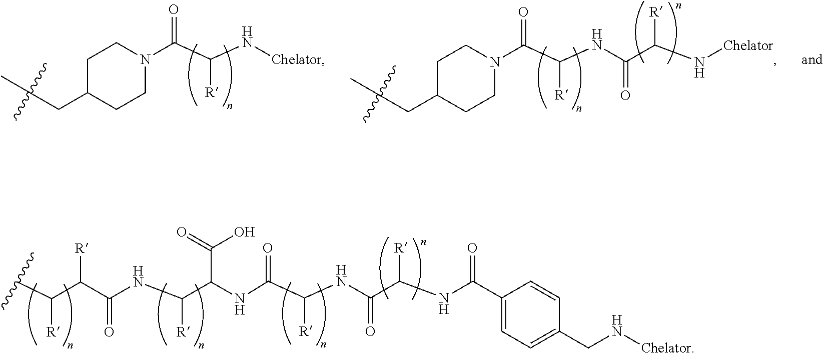

In yet another embodiment of the above formulae, R¹⁰ is selected from the group consisting of an amino acid side chain of Tyr, Lys(DOTA), MAF(DOTA), AEF(DOTA), OMeTyr, Aph(Cbm), and Nle. In still another embodiment, R¹⁰ is the amino acid side chain of Tyr or Aph(Cbm). In an embodiment, R¹⁰ is an amino acid side chain of Tyr. In an embodiment, R¹⁰ is an amino acid side chain of Aph(Cbm).

In an embodiment of the above formulae, R¹² is selected from the group consisting of an amino acid side chain of (2S,3S-betaMe-5FW), 1Nal, 2MeW, 2Nal, 4CIW, 4FW, 4MeOW, 4MeW, 5CIW, 5FW, 5MeOW, 5MeW, 7NW, Ala, aMeW, dAla, hF, HomoArg, HomoGlu, Trp(CH₂COOH), Trp(Me), and Trp. In an embodiment, R¹² is selected from the group consisting of an amino acid side chain of Trp, Lys(DOTA), 5FW, and 1Nal. In another embodiment, R¹² is selected from the group consisting of the amino acid side chain of Trp, 5FW, and 1Nal. In still another embodiment, R¹² is an amino acid side chain of 5FW or 1Nal.

In an embodiment of the above formulae, R¹³ is selected from the group consisting of an amino acid side chain of [(2S, 3R)beta-Me-Phe], [(2S, 3R)beta-OH-Phe], [(2S, 3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, ChA, dAla, Phe, His, HomoArg, HomoGlu, HomoPhe, Leu, Nle, NMeNle, NMePhe, Tbg, and tBuAla. In yet another embodiment of the above formulae, R¹³ is selected from the group consisting of an amino acid side chain of Phe, Nle, and [(2S, 3S)beta-Me-Phe]. In still another embodiment, R¹³ is the amino acid side chain of Phe or [(2S, 3S)beta-Me-Phe].

In an embodiment of the above formulae, R^{14A} is

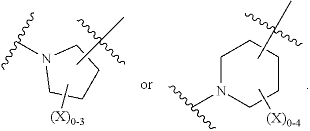

In another embodiment of the above formulae, R^{14A} is

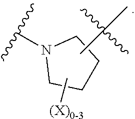

In yet another embodiment of the above formulae, R^{14B} is selected from the group consisting of an amino acid side chain of 4PipAla, alloThr, aMeSer, Dab, Dap, dGlu, dLys, dThr, Glu, gE, hydroxyVal, Lys, NMeSer, Orn, Ser, and Thr. In still another embodiment, R^{14B} is the amino acid side chain of hydroxyVal or Thr.

In an embodiment of the above formulae, R¹⁵ is selected from the group consisting of an amino acid side chain of dAla, dLys, dSer, dGlu, Gly, and betaAla.

In another embodiment of the above formulae, B¹ is CH₂ or C(CH₃)₂; C¹ is CH₂ or C(CH₃)₂; and A¹ is

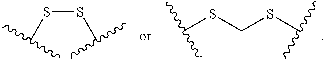

In an embodiment of the above formulae, B¹ is CH₂ or C(CH₃)₂; C¹ is CH₂ or C(CH₃)₂; and A¹ is

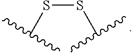 [SMILES: *SS*]

In still another embodiment of the above formulae, B¹ is CH₂; C¹ is CH₂; and A¹ is

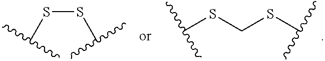

In another embodiment of the above formulae, B¹ is CH₂; C¹ is CH₂; and A¹ is

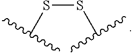 [SMILES: *SS*]

In yet another aspect, provided herein is a cyclic peptide comprising the amino acid sequence of Formula B:

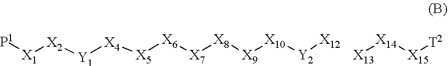

or a pharmaceutically acceptable salt and/or solvate thereof,
wherein:
X₁, X₂, X₅, X₆, X₇, X₉, X₁₀, X₁₂, and X₁₃ are each independently any natural or unnatural amino acids;
Y₁ is Cys or NMeCys;
X₄ is Val or Tbg;
X₈ is Asp or CysAcid;
Y₂ is Cys or NMeCys;
X₁₄ and X₁₅ are each independently any natural or unnatural amino acid or absent;
P¹ is selected from R″, —SO₂R″, -L¹-Chelator,

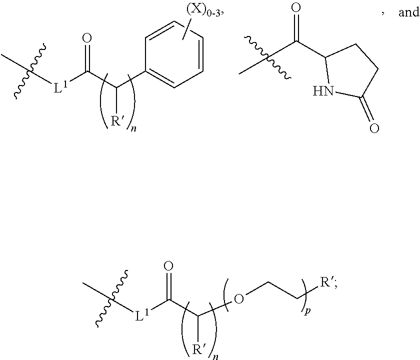

L¹ is absent or selected from

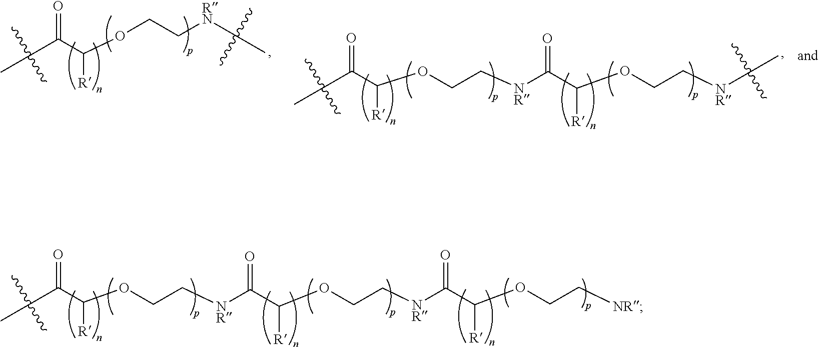

wherein the amino group of L¹ connects to the carbonyl group of P¹ or Chelator to form an amide bond;
T² is R or N(R″)P²;
P² is selected from -L²-Chelator,

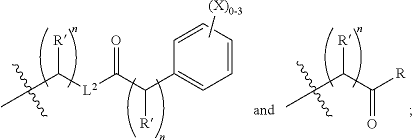

L² is absent or selected from

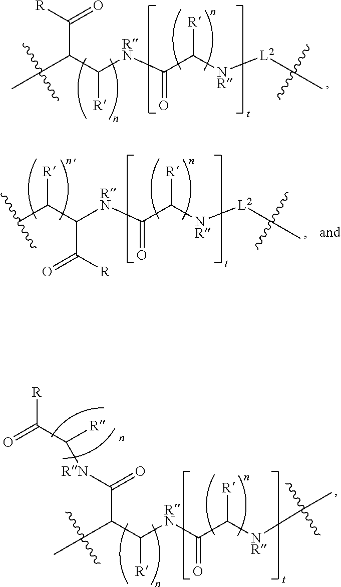

wherein the amino group of L² connects to the carbonyl group of L^{2′} or P² to form an amide bond;
L^{2′} is absent or is selected from

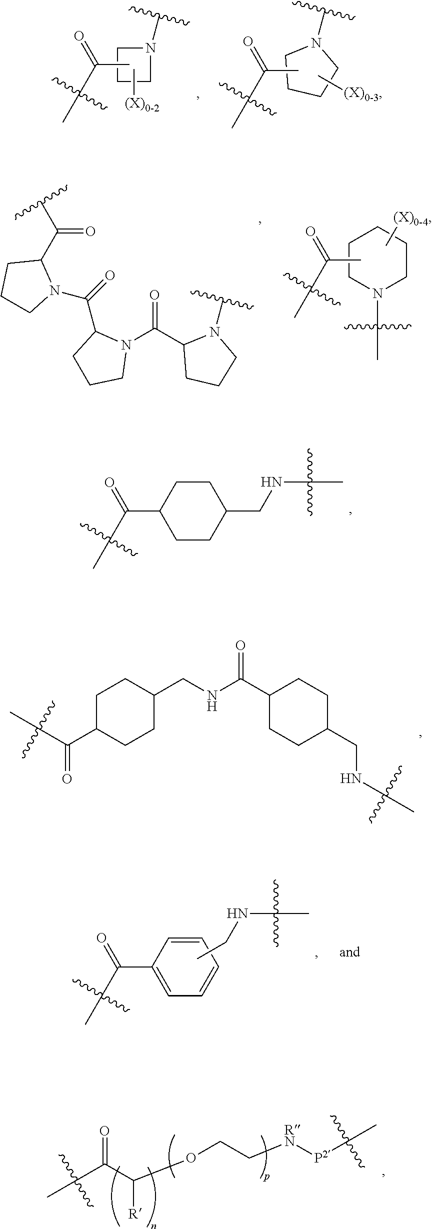

wherein the amino group of L^{2′} connects to the carbonyl group of P^{2′} or Chelator to form an amide bond;
P^{2′} is absent or is

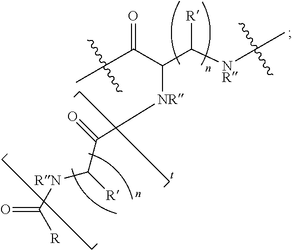

wherein the amino group of P^{2′} connects to the carbonyl group of Chelator to form an amide bond;
each n is independently an integer from 0 to 16;
each p is independently an integer from 0 to 30;
each t is independently 0, 1, 2, 3, 4, 5, or 6;
X is independently, for each occurrence, selected from halo, OH, C₁-C₆ alkyl, and C₁-C₆ haloalkyl;
R is selected from H, C₁-C₂₀ alkyl, C₁-C₆ haloalkyl, OH, —O—(C₁-C₆ alkyl), (C₁-C₆ alkyl)-OH, and N(R″)₂;
R′ is independently, for each occurrence, selected from H, OH, C₁-C₆ alkyl, C₁-C₆ haloalkyl, C₁-C₈ alkylamine, C₁-C₈ alkyl-OH, C(O)H, C(O)(C₁-C₆ alkyl), C(O)OH, C(O)O(C₁-C₆ alkyl), (C₁-C₆ alkyl)C(O)OH, (C₁-C₆ alkyl)C(O)O(C₁-C₆ alkyl), (C₁-C₆ alkyl)CO(C₁-C₆ alkyl), (C₁-C₆ alkyl)CS(C₁-C₆ alkyl), C(O)N(R″)₂, N(R″)₂, and N(R″)₃⁺; and
R″ is independently, for each occurrence, selected from H, C₁-C₆ alkyl, C₁-C₆ haloalkyl, (C₁-C₆ alkyl)C(O)OH;
wherein the cyclic peptide is cyclized via a linker between Y₁ and Y₂; and
wherein the cyclic peptide does not comprise a Chelator or wherein one of P¹, P², X₂, X₁₂, X₁₃, X₁₄, or X₁₅ is substituted with a Chelator, wherein the Chelator is optionally linked to
the cyclic peptide via a linking group.

In an embodiment, the cyclic peptide binds to B7-H3.

In an embodiment, the cyclic peptide does not comprise a Chelator. In another embodiment, the cyclic peptide does not comprise a Chelator or wherein P² is substituted with a Chelator, wherein the Chelator is optionally linked to the cyclic peptide via a linking group.

In another embodiment, the cyclic peptide comprises one Chelator. In yet another embodiment, X₂, X₁₂, X₁₃, X₁₄, or X₁₅ is substituted with a Chelator and an optional linking group, wherein the side chain of the amino acid is selected from L³-Chelator,

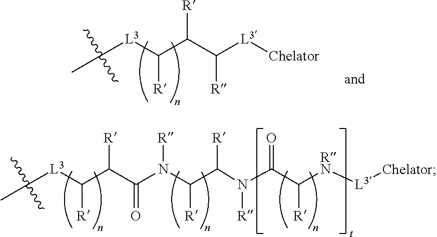

L³ is absent or selected from

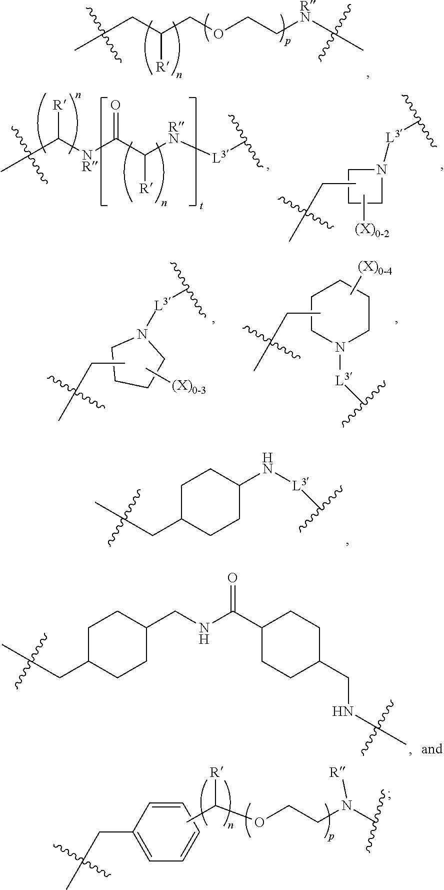

L^{3′} is absent or selected from

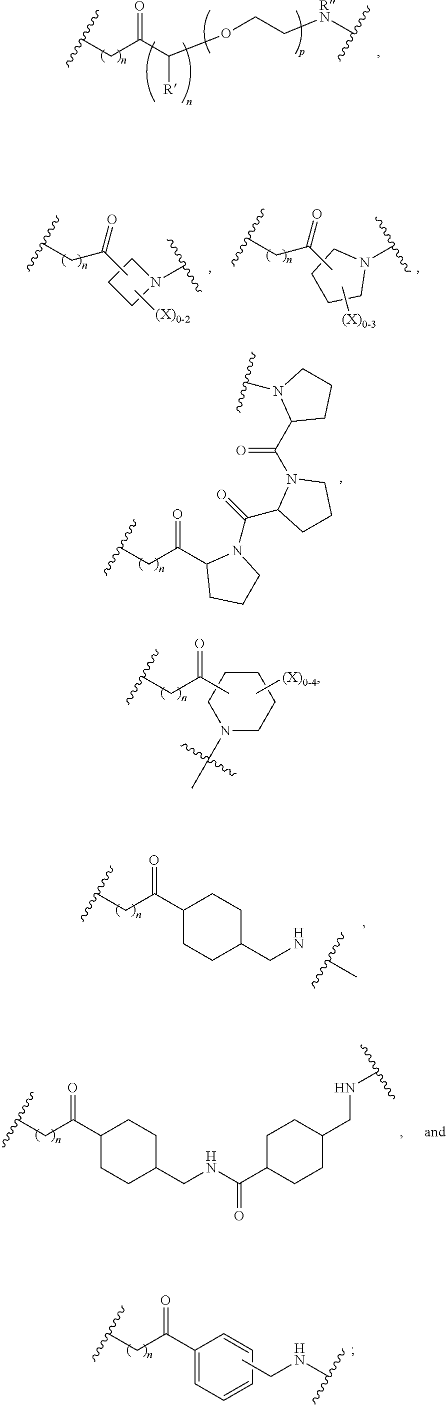

each n is independently an integer from 0 to 16;
each p is independently an integer from 0 to 30;
each t is independently 0, 1, 2, 3, 4, 5, or 6;
X is independently, for each occurrence, selected from halo, OH, C₁-C₆ alkyl, and C₁-C₆ haloalkyl;
R′ is independently, for each occurrence, selected from H, OH, C₁-C₆ alkyl, C₁-C₆ haloalkyl, C₁-C₈ alkylamine, C₁-C₈ alkyl-OH, C(O)H, C(O)(C₁-C₆ alkyl), C(O)OH, C(O)O(C₁-C₆ alkyl), (C₁-C₆ alkyl)C(O)OH, (C₁-C₆ alkyl)C(O)O(C₁-C₆ alkyl), (C₁-C₆ alkyl)CO(C₁-C₆ alkyl), (C₁-C₆ alkyl)CS(C₁-C₆ alkyl), C(O)N(R″)₂, N(R″)₂, and N(R″)₃⁺; and
R″ is independently, for each occurrence, selected from H, C₁-C₆ alkyl, C₁-C₆ haloalkyl, (C₁-C₆ alkyl)C(O)OH.

In still another embodiment, X₁ is hLeu or hCha and X₂ is Tyr.

In another embodiment, X₁ is selected from Ala, Cha, dAla, hCba, hCha, hLeu, HomoArg, HomoGlu, hTba, Leu, Met, and Nle. In yet another embodiment, X₁ is selected from hLeu, hCha, hCba and Nle, In still another embodiment, X₁ is hLeu or hCha.

In another embodiment, X₂ is selected from 3CIY, 3FY, 4COOHPhe, 4FPhe, 4PyA, 7AzaW, Ala, alloThr, aMeNle, aMeTyr, Cba, Chg, dAla, FSY, HomoArg, HomoGlu, hTyr, hyVal, Nle, NMeNle, OMeTyr, Tba, ThpA, ThpG, Thr, and Tyr. In yet another embodiment, X₂ is selected from 4COOHPhe, 4FPhe, aMeTyr, hTyr, OMeTyr, ThpA, and Tyr. In still another embodiment, X₂ is Tyr or 4FPhe. In an embodiment, X₂ is Tyr. X₂ can be substituted with a Chelator, wherein the Chelator is optionally linked to the cyclic peptide via a linking group. In an embodiment, the side chain of X₂ is a structure selected from

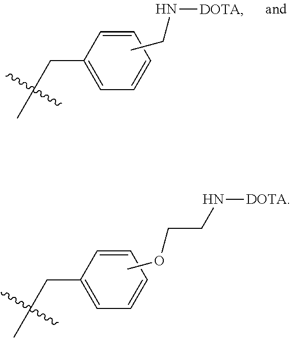

In another embodiment, the linker between Y₁ and Y₂ is selected from a bond, C₁-6 alkylene, and

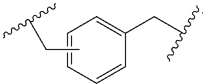

In yet another embodiment, Y₁ is Cys. In still another embodiment, Y₁ is NMeCys.

In another embodiment, Y₂ is Cys. In still another embodiment, Y₂ is NMeCys.

In an embodiment, X₄ is Tbg. In another embodiment, X₄ is Val.

In yet another embodiment, X₅ is selected from Ala, dAla, Gly, HomoGlu, N(CH2)2COOHGly, NetGly, Ser, and Sar. In still another embodiment, X₅ is Gly or Sar. In an embodiment, X₅ is Gly. In another embodiment, X₅ is Sar.

In still another embodiment, X₆ is selected from Ala, aMeLys, Arg, Aze, Cit, Dab, dAla, Dap, dPro, HomoGlu, hSer, Lys, Lys(Ac), Lys(Me)2, Lys(Me)3, Nle, NMeLys, Orn, Pip, Pip(Ac)Ala, Pip(C4diacid)Ala, Pip(CH2COOH)Ala, Pip(CONH2)Ala, Pip(Me)Ala, PipAla, Pro, ThpA, and 4PyA. In an embodiment, X₆ is selected from Lys, PipAla, ThpA, Cit, Pip(CH2COOH)Ala, Lys(Ac), Pip(Ac)Ala, Pip(CONH2)Ala, Pip(Me)Ala, Lys(Me)2, and Lys(Me)3. In another embodiment, X₆ is selected from Lys, Pip(CH2COOH)Ala, and PipAla. In yet another embodiment, X₆ is Pip(CH2COOH)Ala or Lys. In still another embodiment, X₆ is Pip(CH2COOH)Ala.

In an embodiment, the cyclic peptide comprising the amino acid sequence of Formula B is a cyclic peptide comprising the amino acid sequence of Formula Bi:

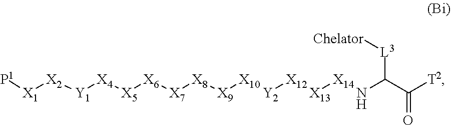

or a pharmaceutically acceptable salt and/or solvate thereof.

In another embodiment, the cyclic peptide comprising the amino acid sequence of Formula B is a cyclic peptide comprising the amino acid sequence of Formula Bii:

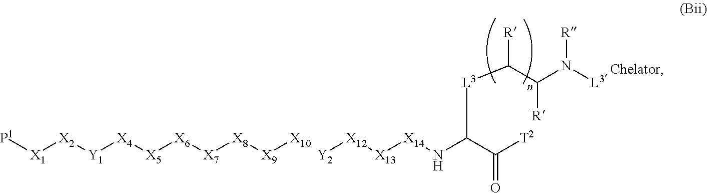

or a pharmaceutically acceptable salt and/or solvate thereof.

In yet another embodiment, the cyclic peptide comprising the amino acid sequence of Formula B is a cyclic peptide comprising the amino acid sequence of Formula Bili:

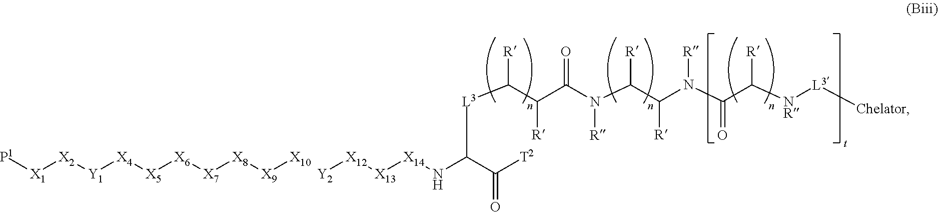

or a pharmaceutically acceptable salt and/or solvate thereof.

In still another embodiment, X₇ is selected from (N-Me-ThpA), 4COOHPhe, 4OHCha, 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, and Tyr. In an embodiment, X₇ is selected from Tyr, Cha, PipAla, 4PyA, ThpA, 4COOHPhe, Pip(CH2COOH)Ala, Pip(DOTA)Ala, and 4OHCha. In another embodiment, X₇ is selected from 4PyA and ThpA. In yet another embodiment, X₇ is ThpA.

In yet another embodiment, the cyclic peptide comprising the amino acid sequence of Formula B is a cyclic peptide comprising the amino acid sequence of Formula Biv:

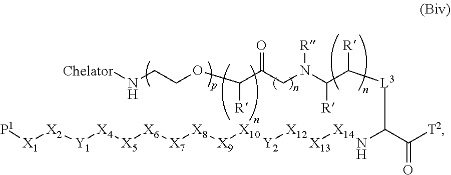

or a pharmaceutically acceptable salt and/or solvate thereof.

In an embodiment, the cyclic peptide comprising the amino acid sequence of Formula B is a cyclic peptide comprising the amino acid sequence of Formula Bv:

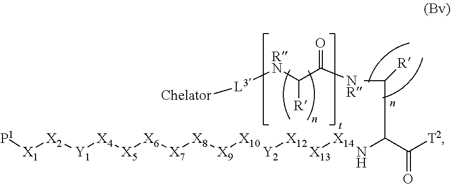

or a pharmaceutically acceptable salt and/or solvate thereof. In still another embodiment, X₈ is Asp. In an embodiment, X₈ is CysAcid.

In another embodiment, X₉ is selected from Abu, Aib, Ala, allolle, alloThr, aMeSer, aMeVal, ChG, Cpg, dAla, HomoGlu, hSer, hydroVal, Nle, NMeVal, Nva, Ser, Tbg, Thr, and Val. In yet another embodiment, X₉ is Val.

In another embodiment, X₁₀ is (4-carbamoyl-Phe), 2OHPhe, 3FY, 3OHPhe, 3PyA, 4FPhe, Ala, alloThr, aMeLeu, aMeTyr, Aph(Cbm), Cha, Chg, CyanoButric, dAla, FSY, HomoArg, HomoGlu, hyVal, Nle, NMeTyr, OMeTyr, Tba, ThpA, Thr, and Tyr. In yet another embodiment, X₁₀ is selected from Tyr, OMeTyr, Aph(Cbm), and Nle. In still another embodiment, X₁₀ is Tyr or Aph(Cbm). In an embodiment, X₁₀ is Tyr or Aph(Cbm). In another embodiment, X₁₀ is Tyr. In yet another embodiment, X₁₀ is Aph(Cbm).

In another embodiment, X₁₂ is selected from (2S,3S-betaMe-5FW), 1Nal, 2MeW, 2Nal, 4CIW, 4FW, 4MeOW, 4MeW, 5CIW, 5FW, 5MeOW, 5MeW, 7NW, Ala, aMeW, dAla, hF, HomoArg, HomoGlu, Trp(CH₂COOH), Trp(Me), and Trp. In yet another embodiment, X₁₂ is selected from Trp, Lys(DOTA), 5FW, and 1Nal. In still another embodiment, X₁₂ is selected from 1Nal, 2MeW, 2Nal, 4CIW, 4FW, 4MeOW, 4MeW, 5CIW, 5FW, 5MeOW, 5MeW, 7NW, aMeW, Trp(CH₂COOH), Trp(Me), and Trp. In an embodiment, X₁₂ is selected from Trp, 5FW, and 1Nal. In another embodiment, X₁₂ is 5FW or 1Nal. X₁₂ can be substituted with a Chelator, wherein the Chelator is optionally linked to the cyclic peptide via a linking group. In an embodiment, the side chain of X₁₂ is the structure:

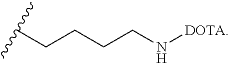

In yet another embodiment, X₁₃ is selected from [(2S, 3R)beta-Me-Phe], [(2S, 3R)beta-OH-Phe], [(2S, 3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, Cha, dAla, Phe, His, HomoArg, HomoGlu, HomoPhe, Leu, Nle, NMeNle, NMePhe, Tbg, and tBuAla. In still another embodiment, X₁₃ is selected from [(2S, 3R)beta-Me-Phe], [(2S,3R)beta-OH-Phe], [(2S,3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, aMeNle, aMePhe, Phe, HomoPhe, and NMePhe. In an embodiment, X₁₃ is selected from [(2S,3R)beta-Me-Phe], [(2S,3R)beta-OH-Phe], [(2S,3S)beta-Me-Phe], 2FPhe, 2MePhe, 3ClPhe, and Phe. In another embodiment, X₁₃ is selected from Phe, Nle, and [(2S,3S)beta-Me-Phe]. In yet another embodiment, X₁₃ is Phe or [(2S,3S)beta-Me-Phe]. X₁₃ can be substituted with a Chelator, wherein the Chelator is optionally linked to the cyclic peptide via a linking group. In an embodiment, the side chain of X₁₃ is the structure:

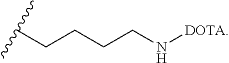

In still another embodiment, X₁₄ is selected from Pro, alpha-Me-Pro, trans4Fluoro-Pro, cis4Fluoro-Pro, trans4OH-Pro, cis4OH-Pro, 5,5-diMe-Pro, trans4NH2-Pro, cis4NH2-Pro, 4PipAla, alloThr, aMeSer, Dab, Dap, dGlu, dLys, dThr, Glu, gE, hydroxyVal, Lys, NMeSer, Orn, Ser, and Thr. In an embodiment, X₁₄ is selected from Pro, alpha-Me-Pro, trans4Fluoro-Pro, cis4Fluoro-Pro, trans4OH-Pro, cis4OH-Pro, 5,5-diMe-Pro, trans4NH2-Pro, cis4NH2-Pro, hydroxyVal, and alloThr, In another embodiment, X₁₄ is selected from hydroxyVal, Pro, and Thr. In yet another embodiment, X₁₄ is hydroxyVal or Thr. In an embodiment, X₁₄ is absent.

In another embodiment, X₁₅ is selected from dAla, dLys, dSer, dGlu, Gly, and betaAla. In yet another embodiment, X₁₅ is selected from dAla, dSer, and dGlu. In an embodiment, X₁₅ is absent. If X₁₄ is absent, then X₁₅ is absent. X₁₅ can be substituted with a Chelator, wherein the Chelator is optionally linked to the cyclic peptide via a linking group. In an embodiment, the side chain of X₁₅ is the structure:

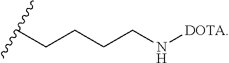

In still another aspect, provided herein is a cyclic peptide of Formula VI:

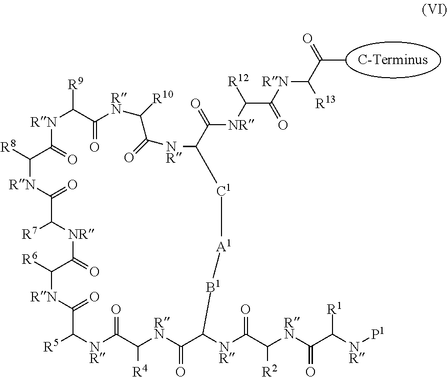

or a pharmaceutically acceptable salt thereof,
wherein:
the C-Terminus is selected from R, N(R″)P²,

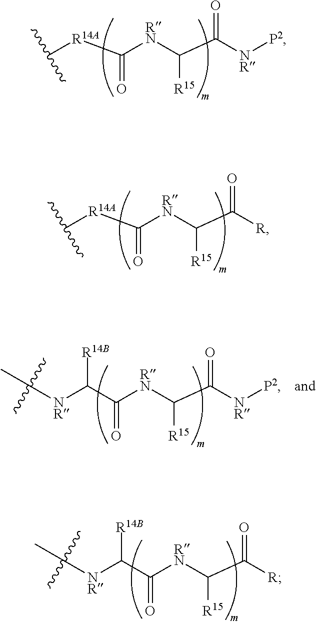

P¹ is selected from R″, —SO₂R″, -L¹-Chelator,

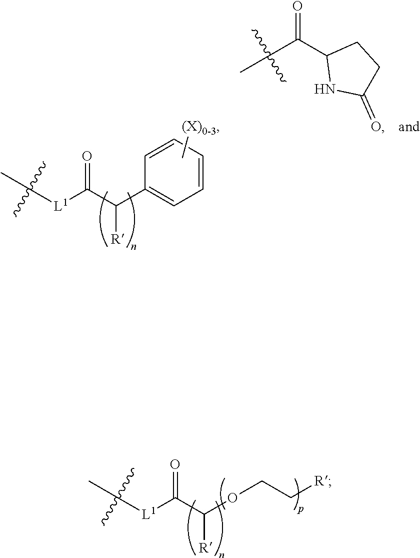

L¹ is absent or selected from

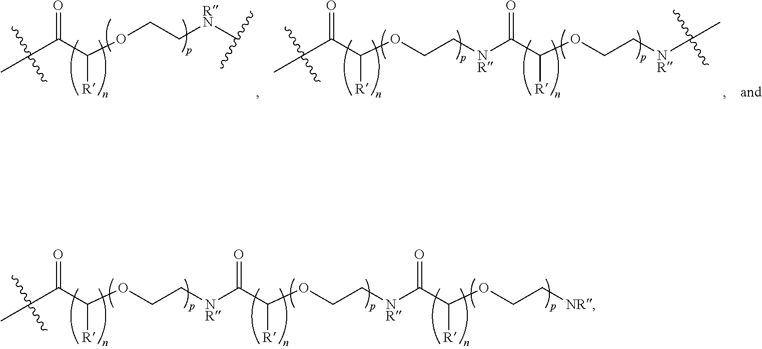

wherein the amino group of L¹ connects to the carbonyl group of P¹ or Chelator to form an amide bond;
P² is selected from -L²-Chelator,

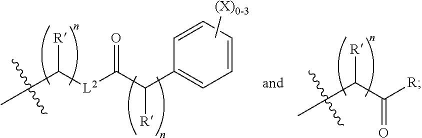

L² is absent or selected from

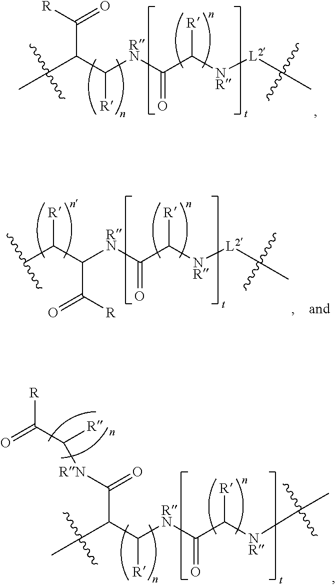

wherein the amino group of L² connects to the carbonyl group of L^{2′} or P² to form an amide bond;
L^{2′} is absent or is selected from

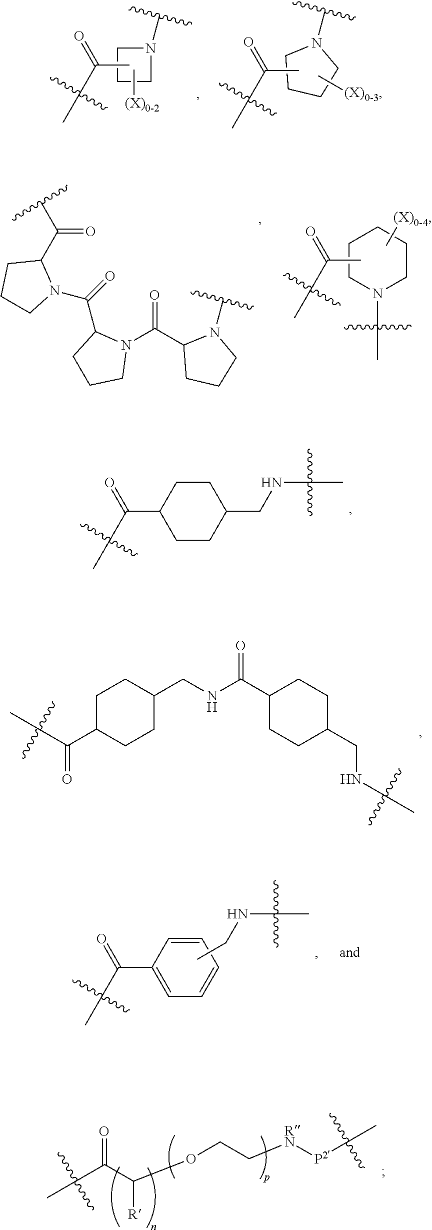

wherein the amino group of L^{2′} connects to the carbonyl group of P^{2′} or Chelator to form an amide bond;
P^{2′} is absent or is

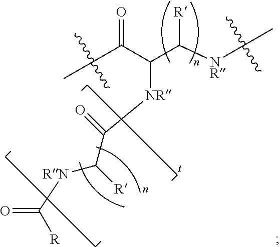

wherein the amino group of P^{2′} connects to the carbonyl group of Chelator to form an amide bond;
R¹ is an amino acid side chain of a natural amino acid or an amino acid side chain of an unnatural amino acid;
R² is selected from the group consisting of:
(i) an amino acid side chain of a natural amino acid,
(ii) an amino acid side chain of an unnatural amino acid,
(iii) L³-Chelator,

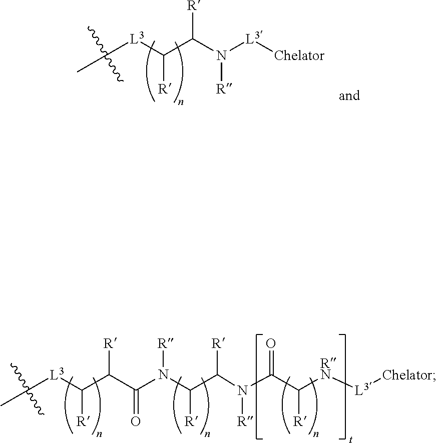

B¹ is C₁-6 alkylene;
C¹ is C₁-6 alkylene;
A¹ is selected from:

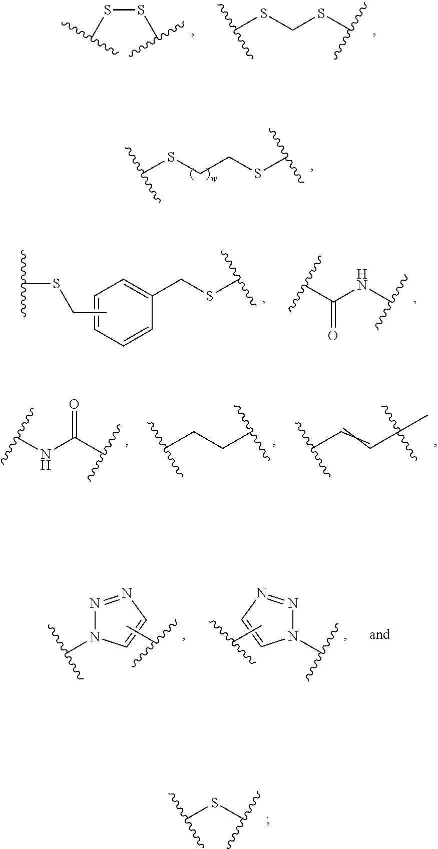

w is 1, 2, or 3;
R⁴ is selected from an amino acid side chain of a natural amino acid and an amino acid side chain of an unnatural amino acid;
R⁵ is selected from an amino acid side chain of a natural amino acid and an amino acid side chain of an unnatural amino acid;
R⁶ is selected from the group consisting of:
(i) an amino acid side chain of a natural amino acid,
(ii) an amino acid side chain of an unnatural amino acid, and
(iii) a structure selected from
1.

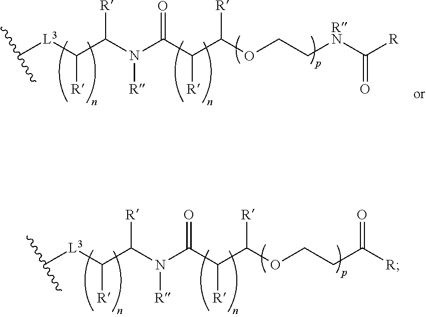

R⁷ is selected from:
(i) an amino acid side chain of a natural amino acid,
(ii) and an amino acid side chain of an unnatural amino acid, and
(iii)

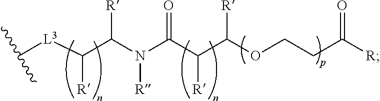

R⁸ is selected from an amino acid side chain of a natural amino acid and an amino acid side chain of an unnatural amino acid;
R⁹ is selected from an amino acid side chain of a natural amino acid and an amino acid side chain of an unnatural amino acid;
R¹⁰ is selected from an amino acid side chain of a natural amino acid and an amino acid side chain of an unnatural amino acid;
R¹² is selected from the group consisting of:
(i) an amino acid side chain of a natural amino acid,
(ii) an amino acid side chain of an unnatural amino acid,
(iii) L³-Chelator,

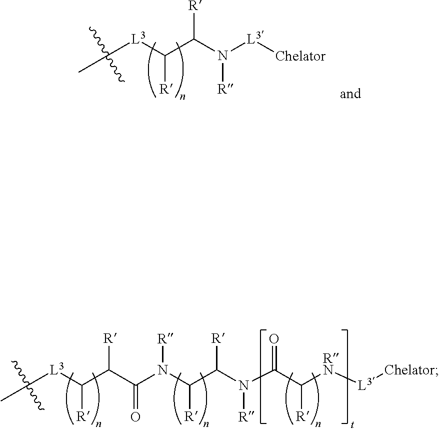

R¹³ is selected from:
(i) an amino acid side chain of a natural amino acid,
(ii) an amino acid side chain of an unnatural amino acid,
(iii) L³-Chelator,

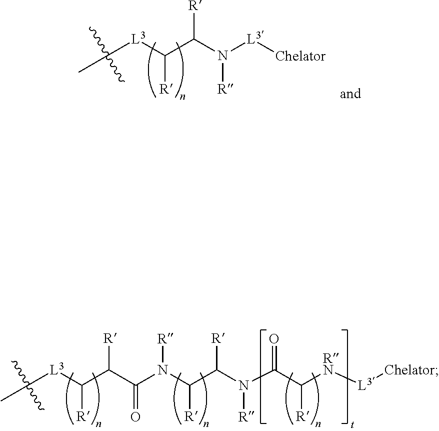

R^{14A} is selected from:

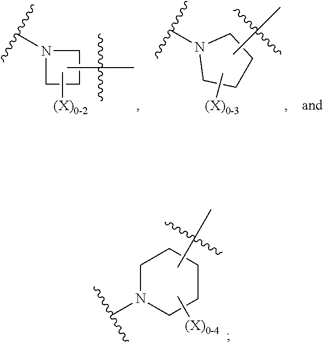

R^{14B} is selected from:
(i) an amino acid side chain of a natural amino acid,
(ii) an amino acid side chain of an unnatural amino acid,
R¹⁵ is an amino acid side chain of a natural amino acid or an amino acid side chain of an unnatural amino acid;
L³ is absent or selected from

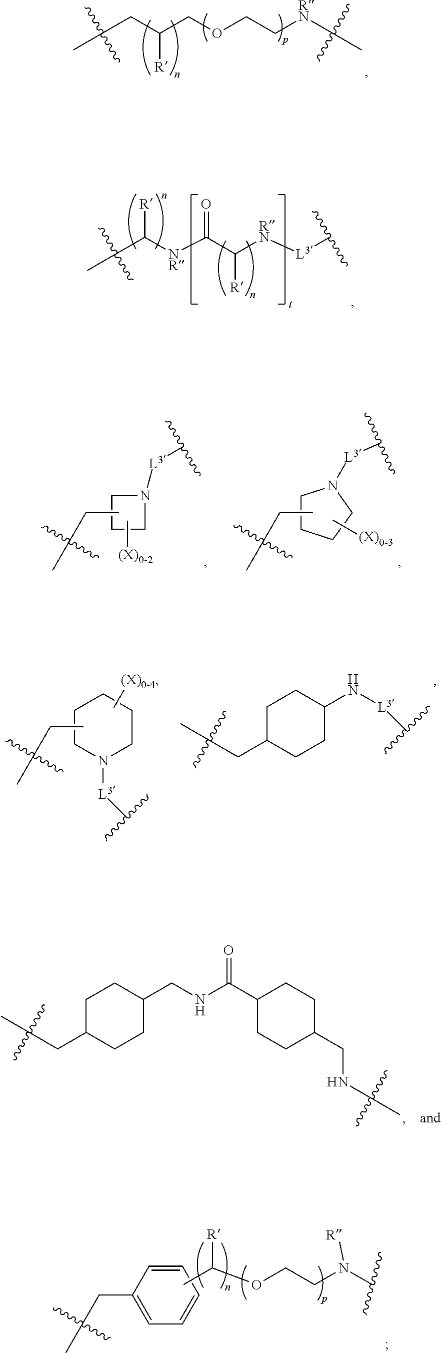

L^{3′} is absent or selected from

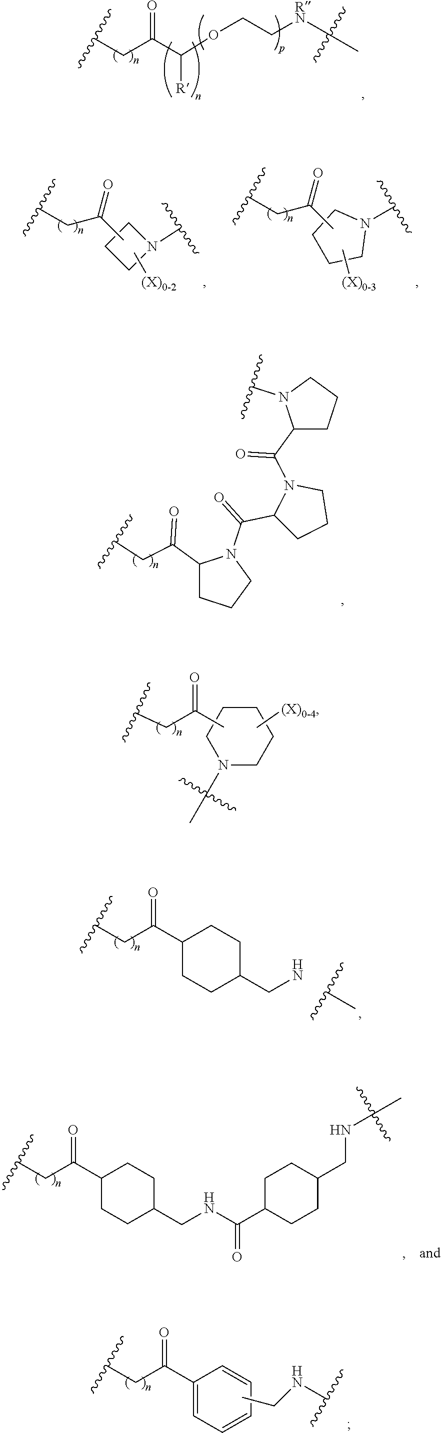

m is 0 or 1;
n′ is independently an integer from 1 to 16;
each n is independently an integer from 0 to 16;
each p is independently an integer from 0 to 30;
each t is independently 0, 1, 2, 3, 4, 5, or 6;
X is independently, for each occurrence, selected from halo, OH, C₁-C₆ alkyl, and C₁-C₆ haloalkyl;
R is independently, for each occurrence, selected from H, C₁-C₂₀ alkyl, C₁-C₆ haloalkyl, OH, —O—(C₁-C₆ alkyl), (C₁-C₆ alkyl)-OH, and N(R″)₂;
R′ is independently, for each occurrence, selected from H, OH, C₁-C₆ alkyl, C₁-C₆ haloalkyl, C₁-C₈ alkylamine, C₁-C₈ alkyl-OH, C(O)H, C(O)(C₁-C₆ alkyl), C(O)OH, C(O)O(C₁-C₆ alkyl), (C₁-C₆ alkyl)C(O)OH, (C₁-C₆ alkyl)C(O)O(C₁-C₆ alkyl), (C₁-C₆ alkyl)CO(C₁-C₆ alkyl), (C₁-C₆ alkyl)CS(C₁-C₆ alkyl), C(O)N(R″)₂, N(R″)₂, and N(R″)₃⁺; and
R″ is independently, for each occurrence, selected from H, C₁-C₆ alkyl, C₁-C₆ haloalkyl, (C₁-C₆ alkyl)C(O)OH, and;
wherein either the cyclic peptide does not comprise a Chelator or comprises one Chelator.

In an embodiment, when a variable group R¹, R², R⁴, R⁵, R⁶, R⁷, R⁸, R⁹, R¹⁰, R¹², R¹³, or R¹⁵ is the amino acid side chain of a cyclic amino acid, the corresponding amino acid nitrogen of the peptide backbone of forms part of the cyclic group;
each alpha-carbon atom on the peptide backbone is optionally substituted with methyl; and wherein the cyclic peptide optionally comprises a radionuclide.

In some embodiments, the cyclic peptides provided herein are cyclic peptides of Formula I, or a pharmaceutically acceptable salt and/or solvate thereof.

In an embodiment, the cyclic peptide of Formula VI is a cyclic peptide of Formula VI′:

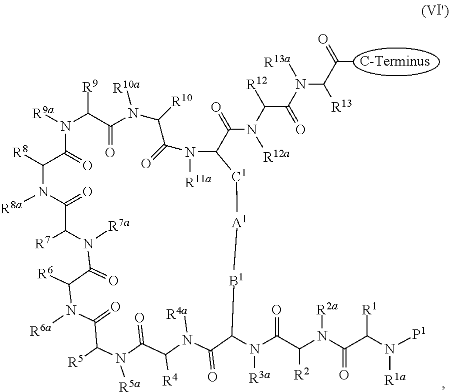

or a pharmaceutically acceptable salt and/or solvate thereof;
wherein
the C-Terminus is selected from R, N(R″)P²,

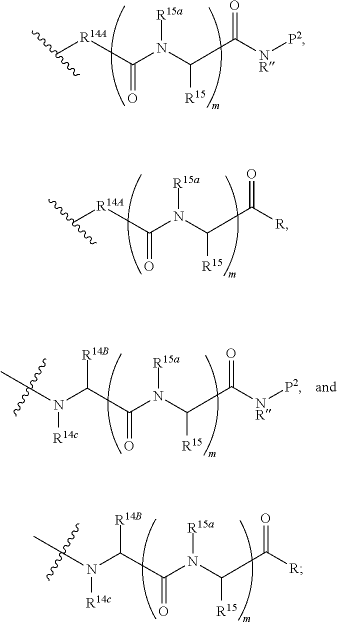

wherein each R^{1a}, R^{2a}, R^{3a}, R^{4a}, R^{5a}, R^{6a}, R^{7a}, R^{8a}, R^{9a}, R^{10a}, R^{11a}, R^{12a}, R^{13a}, R^{15a}, or R^{14c}, is, independently for each occurrence, selected from H and CH₃. In an embodiment, 0, 1, 2, 3, or 4 of R^{1a}, R^{2a}, R^{3a}, R^{4a}, R^{5a}, R^{6a}, R^{7a}, R^{8a}, R^{9a}, R^{10a}, R^{11a}, R^{12a}, R^{13a}, R^{15a}, or R^{14c}, are CH₃.

In an embodiment, the C-Terminus is-L²-Chelator; L² is

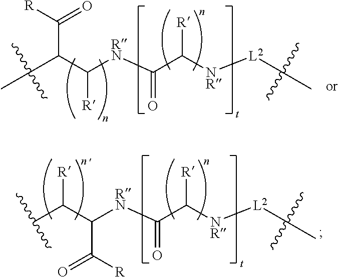

wherein
L^{2′} is absent or

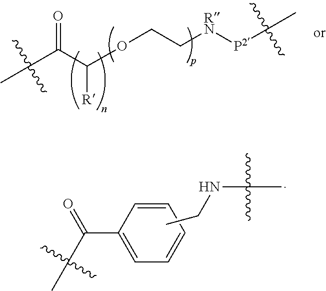

In another embodiment, the cyclic peptide of Formula VI is a cyclic peptide of Formula VIIa:

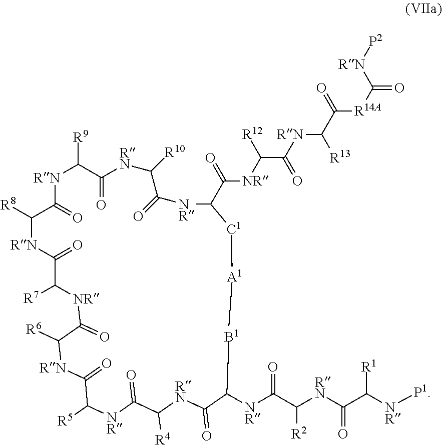

In some embodiments, the cyclic peptides provided herein are cyclic peptides of Formula VIIa, or a pharmaceutically acceptable salt and/or solvate thereof.

In yet another embodiment, the cyclic peptide of Formula I is a cyclic peptide of Formula VIIb:

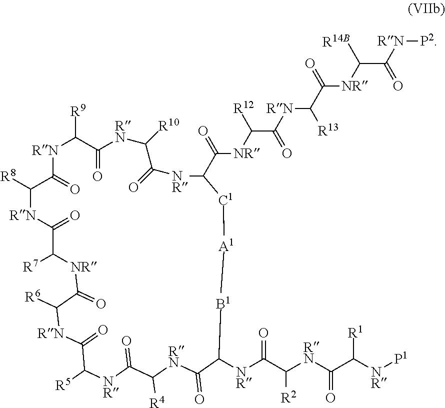

In some embodiments, the cyclic peptides provided herein are cyclic peptides of Formula VIIb, or a pharmaceutically acceptable salt and/or solvate thereof.

In still another embodiment, the cyclic peptide of Formula VI is a cyclic peptide of Formula VIIIa:

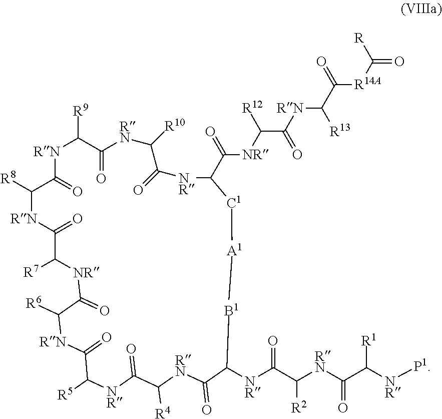

In some embodiments, the cyclic peptides provided herein are cyclic peptides of Formula VIIIa, or a pharmaceutically acceptable salt and/or solvate thereof.

In an embodiment, the cyclic peptide of Formula VI is a cyclic peptide of Formula

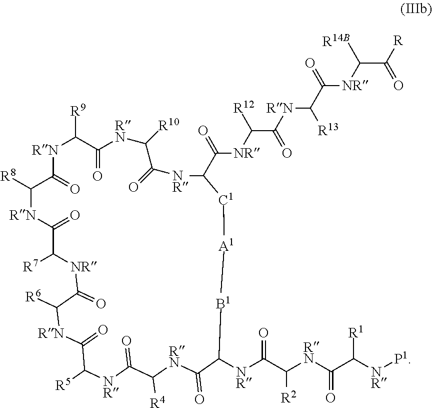

In some embodiments, the cyclic peptides provided herein are cyclic peptides of Formula IIIb, or a pharmaceutically acceptable salt and/or solvate thereof.

In another embodiment, P¹ is selected from —SO₂R″, -L¹-Chelator,

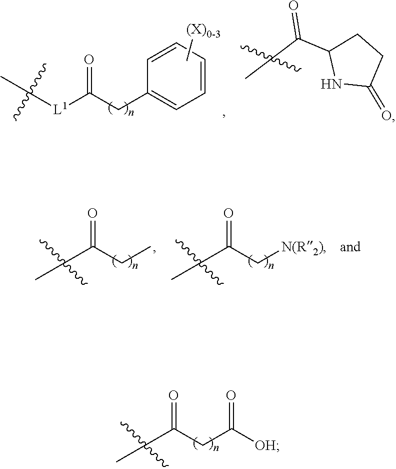

and
n is an integer from 0 to 15; and
R″ is, independently, for each occurrence, H or C₁-C₆ alkyl.

In yet another embodiment, P¹ is-L¹-Chelator; L¹ is absent or is

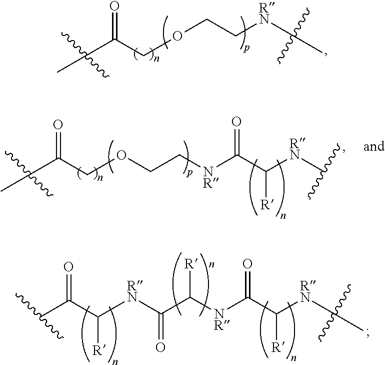

R′ is independently, for each occurrence, selected from H, C₁-C₆ alkyl, C(O)(C₁-C₆ alkyl), C(O)OH, (C₁-C₆ alkyl)C(O)OH, and C(O)N(R″)₂;
R″ is, independently, for each occurrence, H or C₁-C₆ alkyl;
n is an integer from 1 to 15; and
p is an integer from 0 to 12.

In still another embodiment, P¹ is selected from —SO₂R′, Chelator,

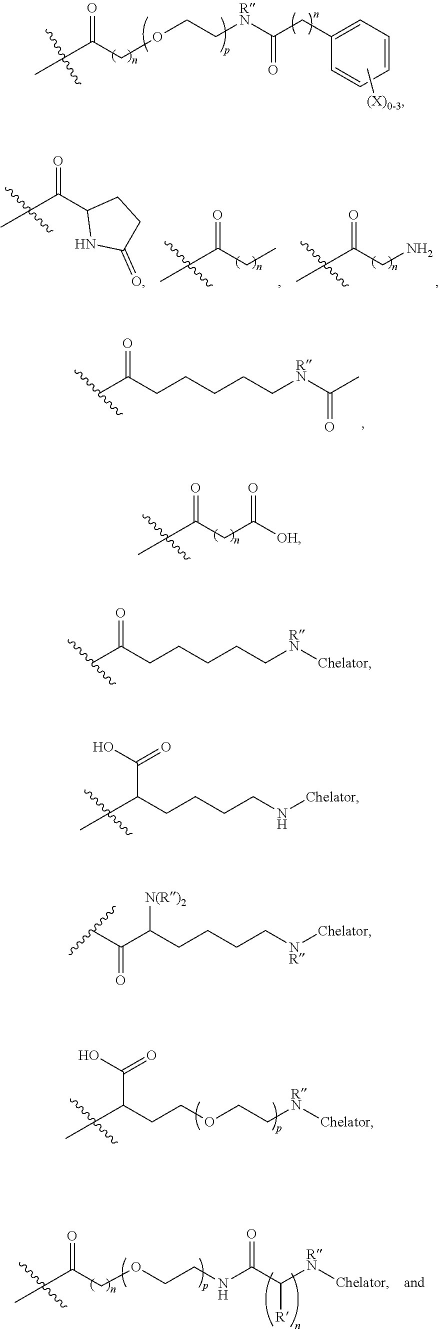

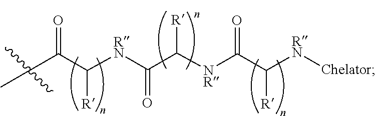

R′ is H, C₁-C₆ alkyl, C(O)(C₁-C₆ alkyl), C(O)OH, (C₁-C₆ alkyl)C(O)OH, and C(O)N(R″)₂;
R″ is independently, for each occurrence, H or C₁-C₆ alkyl;
n is an integer from 1 to 10; and
p is an integer from 1 to 12.

In an embodiment, P¹ is selected from —SO₂Me, Chelator,

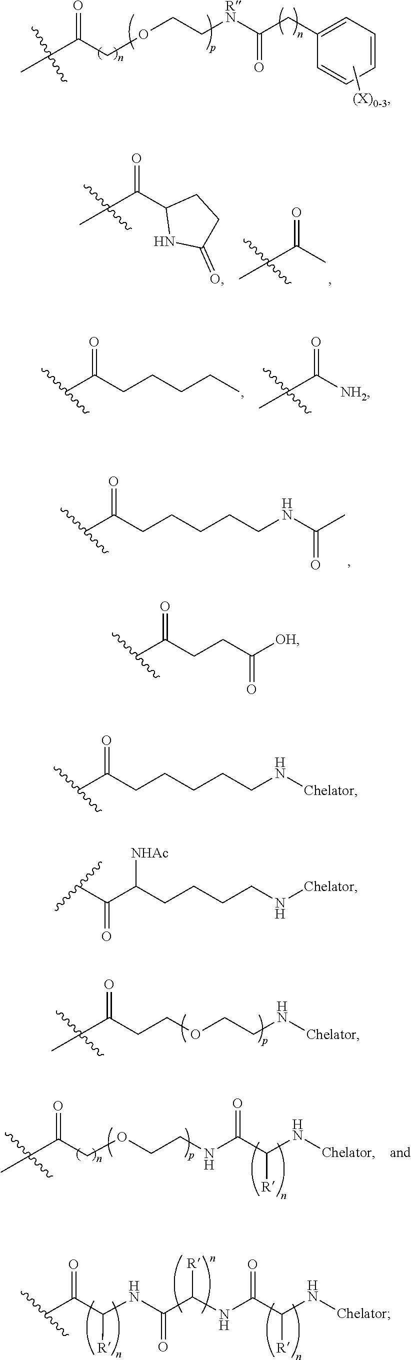

R′ is independently, for each occurrence, selected from H, C(O)OH, (C₁-C₆ alkyl)C(O)OH, and C(O)N(R″)₂;
n is an integer from 1 to 4; and
p is an integer from 1 to 12.

In another embodiment, P¹ is selected from DOTA,

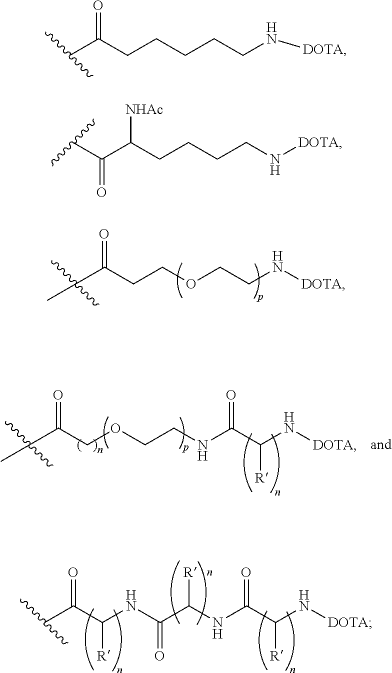

R′ is independently, for each occurrence, selected from H, C(O)OH, (C₁-C₆ alkyl)C(O)OH, and C(O)N(R″)₂;
n is an integer from 1 to 4; and
p is an integer from 1 to 12.

In yet another embodiment, the C-Terminus is selected from OH, N(R″)₂,

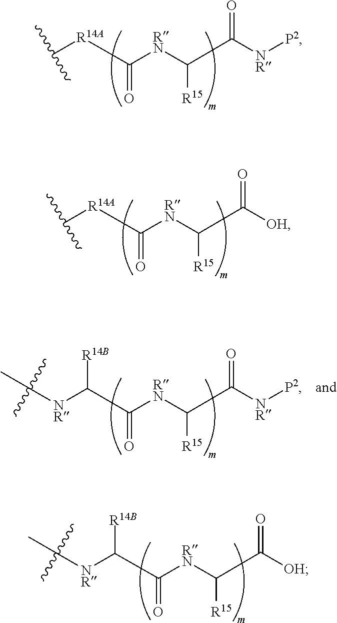

P² is R″ or -L²-Chelator;
L² is selected from

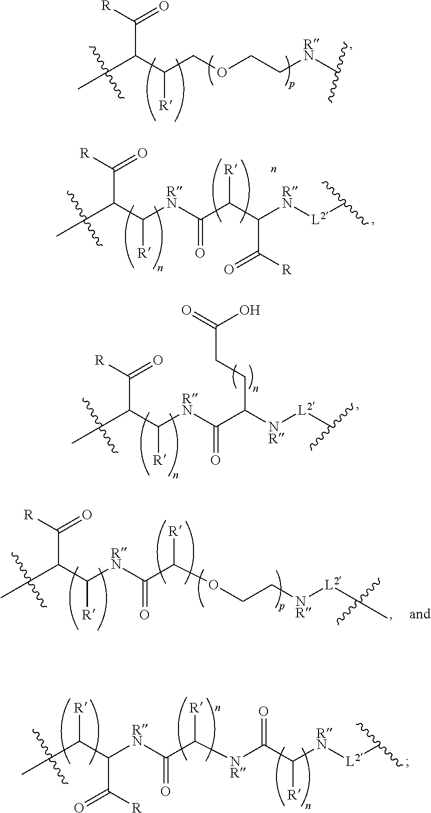

R is selected from C₁-C₆ alkyl, OH, and N(R″)₂;
R′ is individually, for each occurrence, selected from H, C₁-C₆ alkyl, C(O)(C₁-C₆ alkyl), (C₁-C₆ alkyl)S(C₁-C₆ alkyl), C(O)OH, and C(O)N(R″)₂;
R″ is individually, for each occurrence, selected from H and C₁-C₆ alkyl;
n is an integer from 0 to 15; and
p is an integer from 0 to 12.

In still another embodiment, P² is selected from Chelator, N(R″)₂, OH,

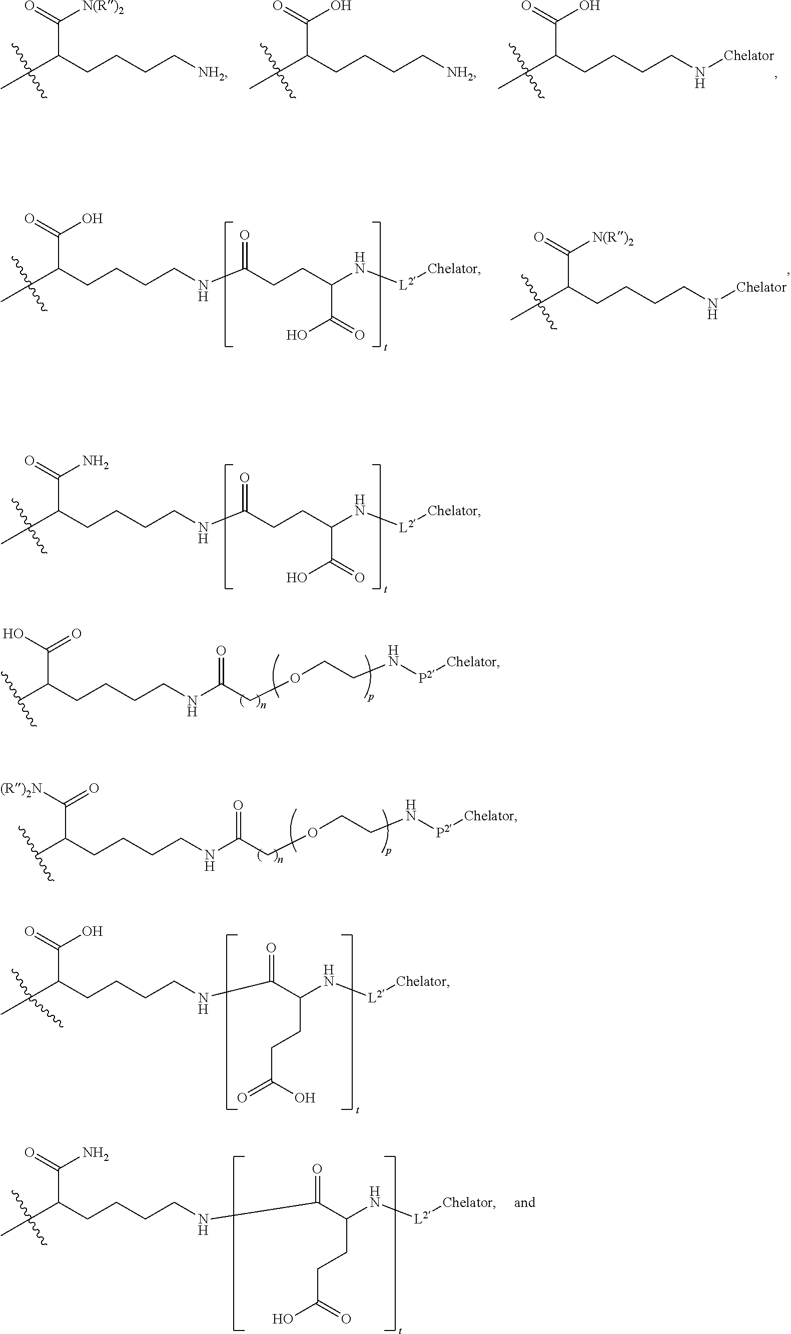

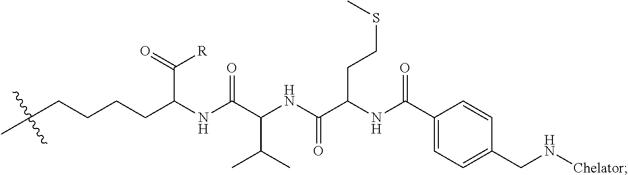

R′ is independently, for each occurrence, H or C₁-C₆ alkyl;
R″ is independently, for each occurrence, H or C₁-C₆ alkyl;
L^{2′} is absent or is

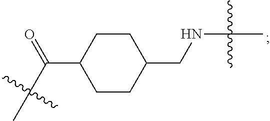 [SMILES: *NCC1CCC(C(*)=O)CC1]

P^{2′} is absent or is

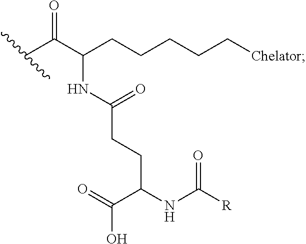

R is C₁-C₂₀ alkyl;
n is an integer from 1 to 4; and
t is an integer from 1 to 3.

In another embodiment, the cyclic peptide of Formula VI is a cyclic peptide of Formula IXa:

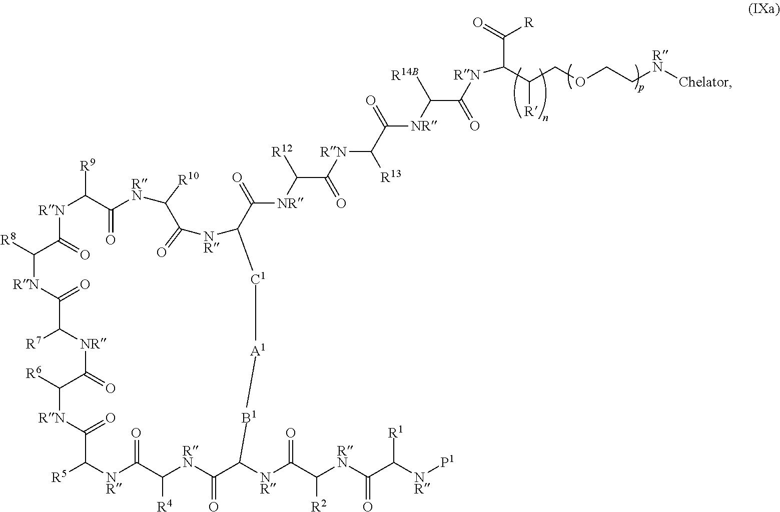

or a pharmaceutically acceptable salt and/or solvate thereof.

In another embodiment, the cyclic peptide of Formula VI is a cyclic peptide of Formula IXb:

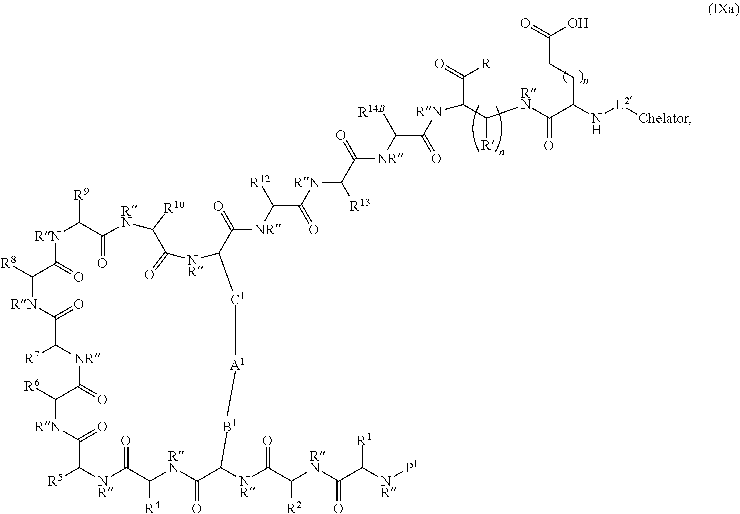

or a pharmaceutically acceptable salt and/or solvate thereof.

In another embodiment, the cyclic peptide of Formula VI is a cyclic peptide of Formula IXc:

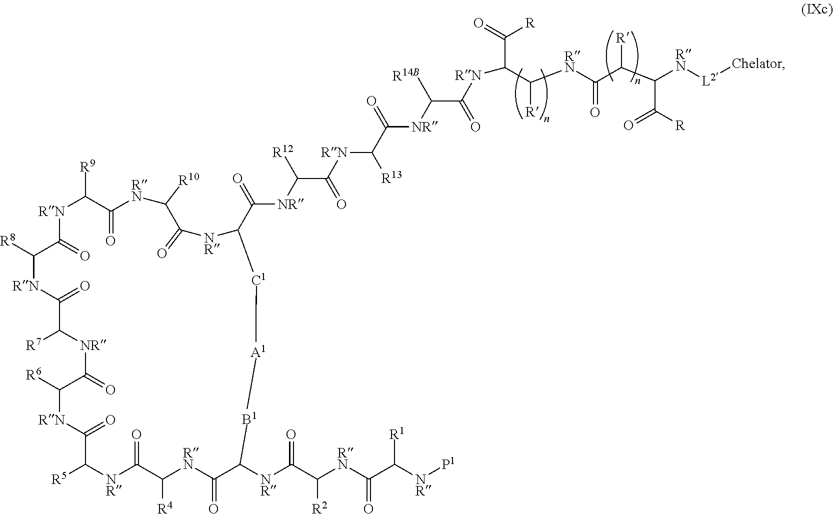

or a pharmaceutically acceptable salt and/or solvate thereof.

In another embodiment, the cyclic peptide of Formula VI is a cyclic peptide of Formula IXd:

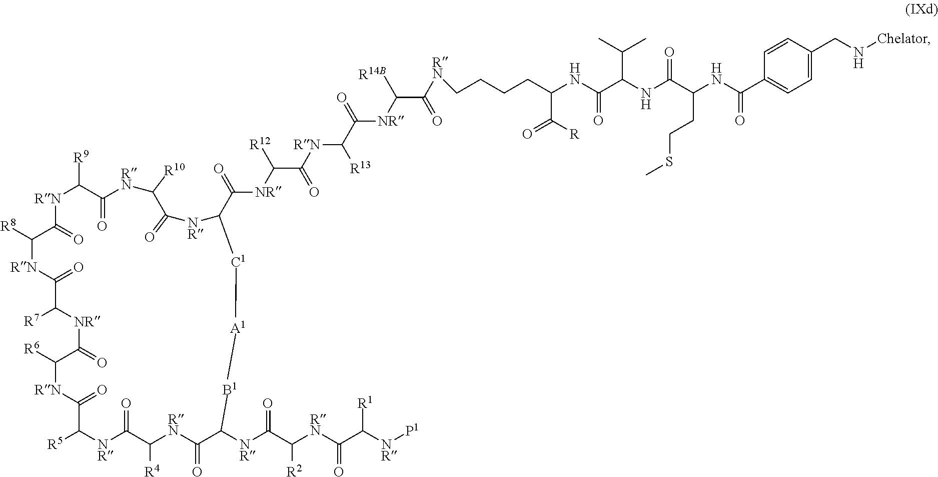

or a pharmaceutically acceptable salt and/or solvate thereof.

In an embodiment, R¹ is selected from the group consisting of an amino acid side chain of Ala, Cha, dAla, hCba, hCha, hLeu, HomoArg, HomoGlu, hTba, Leu, Met, and Nle;
R² is selected from the group consisting of an amino acid side chain of 3CIY, 3FY, 4COOHPhe, 4FPhe, 4PyA, 7AzaW, Ala, alloThr, aMeNle, aMeTyr, Cba, Chg, dAla, FSY, HomoArg, HomoGlu, hTyr, hyVal, Nle, NMeNle, OMeTyr, Tba, ThpA, ThpG, Thr, and Tyr;
R⁴ is selected from the group consisting of an amino acid side chain of Abu, allolle, aMeVal, alloThr, betaMelle, ChG, Cpg, dAla, HomoGlu, hyVal, Ile, Nle, NMeNle, NMeVal, Nva, PipAla, Tbg, tBuAla, Thr, and Val;
R⁵ is selected from the group consisting of an amino acid side chain of Ala, dAla, Gly, HomoGlu, N(CH₂)₂COOHGly, NetGly, Ser, or Sar;
R⁶ is:
(i) selected from the group consisting of an amino acid side chain of Ala, aMeLys, Arg, Aze, Cit, Dab, dAla, Dap, dPro, HomoGlu, hSer, Lys, Lys(Ac), Lys(Me)3, Nle, NMeLys, Orn, Pip, Pip(Ac)Ala, Pip(C4diacid)Ala, Pip(CH2COOH)Ala, Pip(CONH2)Ala, Pip(Me)Ala, PipAla, Pro, ThpA, and 4PyA; or
(ii) the structure:

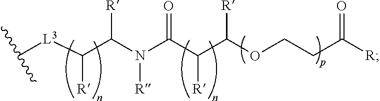

R⁷ is:
(i) selected from the group consisting of an amino acid side chain of (N-Me-ThpA), 4COOHPhe, 4OHCha, 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, and Tyr; or
(ii) the structure:

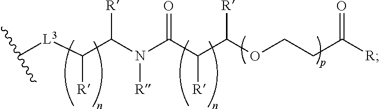

R⁸ is selected from the group consisting of an amino acid side chain of Ala, CysAcid, Asp, Dab, dAla, FSY, HomoGlu, Asn, and Ser;
R⁹ is selected from the group consisting of an amino acid side chain of Abu, Aib, Ala, allolle, alloThr, aMeSer, aMeVal, ChG, Cpg, dAla, HomoGlu, hSer, hydroVal, Nle, NmeVal, Nva, Ser, Tbg, Thr, and Val;
R¹⁰ is selected from the group consisting of an amino acid side chain of (4-carbamoyl-Phe), 2OHPhe, 3FY, 3OHPhe, 3PyA, 4FPhe, Ala, alloThr, aMeLeu, aMeTyr, Aph(Cbm), Cha, Chg, CyanoButric, dAla, FSY, HomoArg, HomoGlu, hyVal, Nle, NMeTyr, OMeTyr, Tba, ThpA, Thr, and Tyr;
R¹² is selected from the group consisting of an amino acid side chain of (2S,3S-betaMe-5FW), 1Nal, 2MeW, 2Nal, 4CIW, 4FW, 4MeOW, 4MeW, 5CIW, 5FW, 5MeOW, 5MeW, 7NW, Ala, aMeW, dAla, hF, HomoArg, HomoGlu, Trp(CH₂COOH), Trp(Me), and Trp; and
R¹³ is selected from the group consisting of an amino acid side chain of [(2S,3R)beta-Me-Phe], [(2S,3R)beta-OH-Phe], [(2S,3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, ChA, dAla, Phe, His, HomoArg, HomoGlu, HomoPhe, Leu, Nle, NMeNle, NMePhe, Tbg, tBuAla; and
R^{14A} is

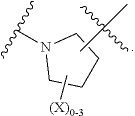

In another embodiment, R¹ is selected from the group consisting of an amino acid side chain of hLeu, hCha, hCba, and Nle;
R² is an amino acid side chain of Tyr;
R⁴ is selected from the group consisting of an amino acid side chain of allolle, Ile, Tbg, and Val;
R⁵ is an amino acid side chain of Gly or Sar;
R⁶ is selected from the group consisting of an amino acid side chain of Dab, Dap, Lys, Pip(CH₂COOH)Ala, and PipAla;
R⁷ is selected from the group consisting of an amino acid side chain of 4COOHPhe, 4OHCha, 4Pya, ThpA, Tyr, and

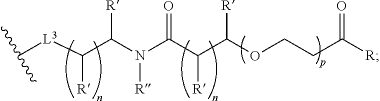

R⁸ is an amino acid side chain of Asp or CysAcid;
R⁹ is an amino acid side chain of ChG or Val;
R¹⁰ is an amino acid side chain of Tyr or Aph(Cbm);
R¹² is selected from the group consisting of an amino acid side chain of 1Nal, 5FW, Trp(Me), and Trp;
R¹³ is selected from the group consisting of an amino acid side chain of Phe, [(2S,3S)beta-Me-Phe], 2MePhe, and 2FPhe; and
R^{14A} is

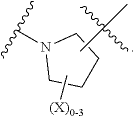

In yet another embodiment, R¹ is selected from the group consisting of an amino acid side chain of Ala, Cha, dAla, hCba, hCha, hLeu, HomoArg, HomoGlu, hTba, Leu, Met, and Nle;
R² is:
(i) selected from the group consisting of an amino acid side chain of 3CIY, 3FY, 4COOHPhe, 4FPhe, 4PyA, 7AzaW, Ala, alloThr, aMeNle, aMeTyr, Cba, Chg, dAla, FSY, HomoArg, HomoGlu, hTyr, hyVal, Nle, NMeNle, OMeTyr, Tba, ThpA, ThpG, Thr, and Tyr; or
(ii) the structure:

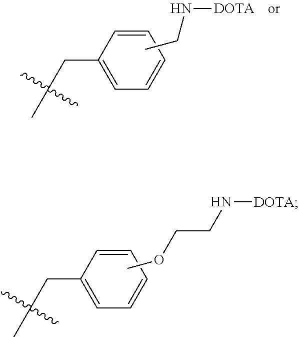

R⁴ is selected from the group consisting of an amino acid side chain of Abu, allolle, aMeVal, alloThr, betaMelle, ChG, Cpg, dAla, HomoGlu, hyVal, Ile, Nle, NMeNle, NMeVal, Nva, PipAla, Tbg, tBuAla, Thr, and Val;
R⁵ is selected from the group consisting of an amino acid side chain of Ala, dAla, Gly, HomoGlu, N(CH₂)₂COOHGly, NetGly, Ser, or Sar;
R⁶ is:
(i) selected from the group consisting of an amino acid side chain of Ala, aMeLys, Arg, Aze, Cit, Dab, dAla, Dap, dPro, HomoGlu, hSer, Lys, Lys(Ac), Lys(Me)3, Nle, NMeLys, Orn, Pip, Pip(Ac)Ala, Pip(C4diacid)Ala, Pip(CH2COOH)Ala, Pip(CONH2)Ala, Pip(Me)Ala, PipAla, Pro, ThpA, and 4PyA; or
(ii) the structure:

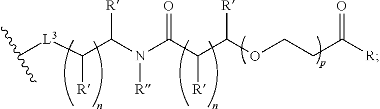

R⁷ is:
(i) selected from the group consisting of an amino acid side chain of (N-Me-ThpA), 4COOHPhe, 4OHCha (pk1), 4OHCha (pk2), 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, and Tyr; or
(ii) the structure:

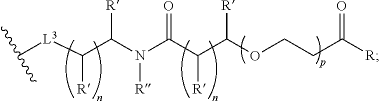

R⁸ is selected from the group consisting of an amino acid side chain of Ala, CysAcid, Asp, Dab, dAla, FSY, HomoGlu, Asn, and Ser;
R⁹ is selected from the group consisting of an amino acid side chain of Abu, Aib, Ala, allolle, alloThr, aMeSer, aMeVal, ChG, Cpg, dAla, HomoGlu, hSer, hydroVal, Nle, NmeVal, Nva, Ser, Tbg, Thr, and Val;
R¹⁰ is selected from the group consisting of an amino acid side chain of (4-carbamoyl-Phe), 2OHPhe, 3FY, 3OHPhe, 3PyA, 4FPhe, Ala, alloThr, aMeLeu, aMeTyr, Aph(Cbm), Cha, Chg, CyanoButric, dAla, FSY, HomoArg, HomoGlu, hyVal, Nle, NMeTyr, OMeTyr, Tba, ThpA, Thr, and Tyr;
R¹² is:
(i) selected from the group consisting of an amino acid side chain of (2S,3S-betaMe-5FW), 1Nal, 2MeW, 2Nal, 4CIW, 4FW, 4MeOW, 4MeW, 5CIW, 5FW, 5MeOW, 5MeW, 7NW, Ala, aMeW, dAla, hF, HomoArg, HomoGlu, Trp(CH₂COOH), Trp(Me), and Trp; or
(ii) the structure:

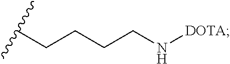

R¹³ is:
(i) selected from the group consisting of an amino acid side chain of [(2S,3R)beta-Me-Phe], [(2S,3R)beta-OH-Phe], [(2S,3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, ChA, dAla, Phe, His, HomoArg, HomoGlu, HomoPhe, Leu, Nle, NMeNle, NMePhe, Tbg, and tBuAla; or
(ii) the structure:

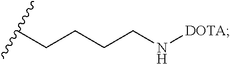

R^{14B} is selected from the group consisting of an amino acid side chain of 4PipAla, alloThr, aMeSer, Dab, Dap, dGlu, dLys, dThr, Glu, gE, hydroxyVal, Lys, NMeSer, Orn, Ser, and Thr; and
R¹⁵ is selected from the group consisting of an amino acid side chain of dAla, dLys, dSer, dGlu, Gly, and betaAla.

In still another embodiment, R¹ is selected from the group consisting of an amino acid side chain of hCba, hCha, hLeu, and Nle;
R² is an amino acid side chain of Tyr;
R⁴ is selected from the group consisting of an amino acid side chain of allolle, betaMelle, Ile, Tbg, and Val;
R⁵ is an amino acid side chain of Gly or Sar;
R⁶ is selected from the group consisting of an amino acid side chain of PipAla, Pip(CH2COOH)Ala, and Lys;
R⁷ is selected from the group consisting of an amino acid side chain of 4COOHPhe, 4Pya, PipAla, ThpA, and Tyr;
R⁸ is an amino acid side chain of Asp or CysAcid;
R⁹ is an amino acid side chain of ChG or Val;
R¹⁰ is an amino acid side chain of Tyr;
R¹² is selected from the group consisting of an amino acid side chain of 1Nal, 5FW, and Trp;
R¹³ is an amino acid side chain of Phe or [(2S,3S)beta-Me-Phe]; and
R^{14B} is selected from the group consisting of an amino acid side chain of alloThr, hydroxyVal, and Thr.

In still another embodiment, R¹ is selected from the group consisting of an amino acid side chain of Ala, Cha, dAla, hCba, hCha, hLeu, HomoArg, HomoGlu, hTba, Leu, Met, and Nle. In an embodiment, R¹ is selected from the group consisting of an amino acid side chain of hLeu, Leu, hCha, hCba, Nle, hTba, HomoArg, HomoGlu, Ala, dAla, and Met. In another embodiment, R¹ is selected from the group consisting of an amino acid side chain of Met, Nle, hLeu, hCha, hCba, Leu, and Nle. In yet another embodiment, R¹ is selected from the group consisting of an amino acid side chain of hLeu, hCha, hCba, and Nle. In still another embodiment, R¹ is an amino acid side chain of hLeu.

In still another embodiment, R² is selected from the group consisting of an amino acid side chain of 3CIY, 4FY, 4COOHPhe, 4FPhe, 4PyA, 7AzaW, Ala, alloThr, aMeNle, aMeTyr, Cba, Chg, dA, FSY, HomoArg, HomoGlu, hTyr, hyVal, Nle, NmeNle, OmeTyr, Tba, ThpA, ThpG, Thr, and Tyr. In an embodiment, R² is an amino acid side chain of Tyr.

In yet another embodiment, R⁴ is selected from the group consisting of an amino acid side chain of Abu, allolle, aMeVal, alloThr, betaMelle, ChG, Cpg, dAla, HomoGlu, hyVal, Ile, Nle, NMeNle, NMeVal, Nva, PipAla, Tbg, tBuAla, Thr, and Val. In another embodiment, R⁴ is selected from the group consisting of an amino acid side chain of Val, dAla, ChG, Tbg, Abu, NmeVal, HomoGlu, Cpg, alloThr, allolle, hydroVal, tBuAla, Nva, Nle, Ile, aMeVal, Thr, and betaMelle. In still another embodiment, R⁴ is selected from the group consisting of an amino acid side chain of allolle, Ile, Tbg, and Val. In yet another embodiment, R⁴ is an amino acid side chain of Val or Tbg.

In another embodiment, R⁵ is selected from the group consisting of an amino acid side chain of Ala, dAla, Gly, HomoGlu, N(CH₂)₂COOHGly, NetGly, Ser, or Sar. In still another embodiment, R⁵ is selected from the group consisting of an amino acid side chain of Gly, dAla, Sar, Ala, NetGly, N(CH₂)₂COOHGly, and HomoGlu. In another embodiment, R⁵ is an amino acid side chain of Gly or Sar. In an embodiment, R⁵ is an amino acid side chain of Gly.

In yet another embodiment, R⁶ is:
(i) selected from the group consisting of an amino acid side chain of Ala, aMeLys, Arg, Aze, Cit, Dab, dAla, Dap, dPro, HomoGlu, hSer, Lys, Lys(Ac), Lys(Me)3, Nle, NMeLys, Orn, Pip, Pip(Ac)Ala, Pip(C4diacid)Ala, Pip(CH2COOH)Ala, Pip(CONH2)Ala, Pip(Me)Ala, PipAla, Pro, ThpA, and 4PyA; or
(ii) the structure:

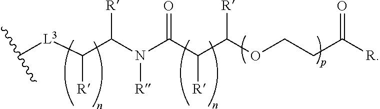

In another embodiment, R⁶ is selected from the group consisting of an amino acid side chain of PipAla, Pip(CH2COOH)Ala, Pip(C4diacid)Ala, Lys, Pya, Ala, aMeLys, Arg, Aze, Dab, dAla, Dap, dPro, HomoGlu, hSer, K(PEG21-COOH), K(yE-yE-yE-C18OH), Lys(Ac), Nle, NmeLys, Orn, Pip, Pip(PEG4-AB)Ala, and Pro. In still another embodiment, R⁶ is selected from the group consisting of an amino acid side chain of Dab, Dap, Lys, Pip(CH2COOH)Ala, and PipAla. In yet another embodiment, R⁶ is selected from the group consisting of an amino acid side chain of PipAla, Pip(CH2COOH)Ala, and Lys.

In another embodiment, R⁷ is:
(i) selected from the group consisting of an amino acid side chain of (N-Me-ThpA), 4COOHPhe, 4OHCha, 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, and Tyr; or
(ii) the structure:

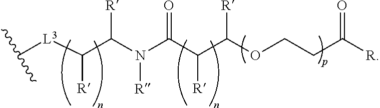

In still another embodiment, R⁷ is selected from the group consisting of an amino acid side chain of Lys, Ala, dAla, 4COOHPhe, 4PyA, HomoGlu, PipAla, hTyr, alloThr, hSer, ThpA, ChA, aMeLeu, Pro, 4OHCha (pk1), 4OHCha (pk2), (N-Me-ThpA), Pip(PEG4-Ac)Ala, Pip(C4diacid)Ala, and Pip(PEG4-C₄diacid)Ala. In yet another embodiment R⁷ is:
(i) selected from the group consisting of an amino acid side chain of 4COOHPhe, 4OHCha, 4Pya, ThpA, and Tyr; or
(ii) the structure:

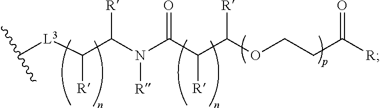

In an embodiment, R⁷ is an amino acid side chain of Lys or ThpA.

In yet another embodiment, R⁸ is selected from the group consisting of an amino acid side chain of Ala, CysAcid, Asp, Dab, dAla, FSY, HomoGlu, Asn, and Ser. In another embodiment, R⁸ is selected from the group consisting of an amino acid side chain of Asp, Ala, dAla, Asn, Ser, HomoGlu, FSY, CysAcid, and Dab. In yet another embodiment, R⁸ is an amino acid side chain of Asp or CysAcid.

In an embodiment, R⁹ is selected from the group consisting of an amino acid side chain of Abu, Aib, Ala, allolle, alloThr, aMeSer, aMeVal, ChG, Cpg, dAla, HomoGlu, hSer, hydroVal, Nle, NmeVal, Nva, Ser, Tbg, Thr, and Val. In still another embodiment, R⁹ is selected from the group consisting of an amino acid side chain of Val, aMeVal, hSer, Thr, Ser, ChG, and aMeSer. In yet another embodiment, R⁹ is an amino acid side chain of ChG or Val. In an embodiment, R⁹ is an amino acid side chain of Val.

In another embodiment, R¹⁰ is selected from the group consisting of an amino acid side chain of Tyr, Ala, dAla, HomoArg, HomoGlu, 3FY, 3PyA, OMeTyr, 4FPhe, Cha, CyanoButric, Thr, ThpA, Tba, Nle, alloThr, hyVal, Chg, NMeTyr, aMeTyr, aMeLeu, FSY, 3OHPhe, (4-carbamoyl-Phe), Aph(Cbm), and 2OHPhe. In still another embodiment, R¹⁰ is an amino acid side chain of Tyr or Aph(Cbm). In yet another embodiment, R¹⁰ is an amino acid side chain of Tyr.

In still another embodiment, R¹² is selected from the group consisting of an amino acid side chain of Trp, Ala, (2S,3S-betaMe-5FW), 1Nal, 2MeW, 2Nal, 4CIW, 4FW, 4MeOW, 4MeW, 5CIW, 5FW, 5MeOW, 5MeW, 7NW, aMeW, dAla, hF, HomoArg, HomoGlu, Trp(CH₂COOH), and TrpMe. In yet another embodiment, R¹² is selected from the group consisting of an amino acid side chain of 1Nal, 5FW, Trp(Me), and Trp. In an embodiment, R¹² is selected from the group consisting of an amino acid side chain of 1Nal, 5FW, and Trp. In another embodiment, R¹² is an amino acid side chain of Trp or 5FW.

In still another embodiment, R¹³ is selected from the group consisting of an amino acid side chain of [(2S,3R)beta-Me-Phe], [(2S,3R)beta-OH-Phe], [(2S,3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, ChA, dAla, Phe, His, HomoArg, HomoGlu, HomoPhe, Leu, Nle, NMeNle, NMePhe, Tbg, and tBuAla. In yet another embodiment, R¹³ is selected from the group consisting of an amino acid side chain of Phe, Ala, dAla, homoPhe, 4CNPhe, 3CNPhe, ChA, 3aminomethylPhe, 4aminomethylPhe, HomoArg, and HomoGlu. In another embodiment, R¹³ is selected from the group consisting of an amino acid side chain of Phe, [(2S, 3S)beta-Me-Phe], 2MePhe, and 2FPhe. In still another embodiment, R¹³ is an amino acid side chain of Phe.

In an embodiment, R^{14A} is

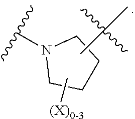

In another embodiment, R^{14B} is selected from the group consisting of an amino acid side chain of 4PipAla, alloThr, aMeSer, Dab, Dap, dGlu, dLys, dThr, Glu, gE, hydroxyVal, Lys, NMeSer, Orn, Ser, and Thr. In yet another embodiment, R^{14B} is selected from the group consisting of an amino acid side chain of alloThr, hydroxyVal, and Thr.

In still another embodiment, R¹⁵ is selected from the group consisting of an amino acid side chain of dAla, dLys, dSer, dGlu, Gly, and betaAla.

In another embodiment, B¹ is CH₂ or C(CH₃)₂; C¹ is CH₂ or C(CH₃)₂; and A¹ is

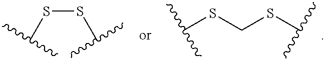

In an embodiment, B¹ is CH₂ or C(CH₃)₂; C¹ is CH₂ or C(CH₃)₂; and A¹ is

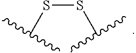 [SMILES: *SS*]

In still another embodiment, B¹ is CH₂; C¹ is CH₂; and A¹ is

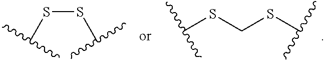

In another embodiment, B¹ is CH₂; C¹ is CH₂; and A¹ is

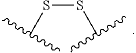 [SMILES: *SS*]

In yet another embodiment of the above formulae, the chelator is independently selected from a group consisting of ethylenediamine tetraacetic acid (EDTA), diethylenetriamine pentaacetic acid (DTPA), 1,4,7,10-tetra-azacylcododecane-N,N′,N″,N″-tetraacetic acid (DOTA), 6-((16-((6-Carboxypyridin-2-yl)methyl)-1,4,10,13-tetraoxa-7,16-diazacyclooctadecan-7-yl)methyl)-4-isothiocyanatopicolinic acid (Macropa), Macrodipa, 2,2′,2″,2′-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, -(2-carboxyethyl) (DOTAGA), 1,4,7-Triazacyclononane-N,N′,N″-triacetic acid (NOTA), 1,4,7,10-tetraazacyclododecane-N,N′,N″,N″-tetraacetic acid (TETA), 1,4,7,10,13-pentaazacyclopentadecane-N,N′,N″,N″,N″-pentaacetic acid (PEPA), 1,4,7,10,13,16-hexaazacyclohexadecane-N, N′,N″,N″,N″,N′″-hexaacetic acid (HEHA), N′-[5-(Acetyl-hydroxy-amino)pentyl]-N-[5-[3-(5-aminopentyl-hydroxy-carbamoyl)propanoylamino]pentyl]-N-hydroxy-butane diamide (DFO), and 1-(1-carboxy-3-carboxypropyl)-4,7-bis-(carboxymethyl)-1,4,7-triazacyclononane (NODAGA).

In yet another embodiment of the above formulae, the chelator is selected from a chelator in Table 3.

In still another embodiment of the above formulae, the chelator is DOTA. In an embodiment, the chelator is DOTAGA. In another embodiment, the chelator is Macrodipa. In yet another embodiment, the chelator is macropa. In still another embodiment, the chelator is DTPA.

In the formulae provided herein, a variable, e.g., an R¹, R², R⁴, R⁵, R⁶, R⁷, R⁸, R⁹, R¹⁰, R¹², R¹³, R^{14A}, or R¹⁵ group, can be defined as the side chain of a cyclic amino acid, e.g., proline. In that instance, the corresponding amino acid nitrogen of the peptide backbone of the generic formula provided herein forms part of the cyclic group. For example, “R⁹ is selected from the group consisting of an amino acid side chain of Pro, alpha-Me-Pro, trans4Fluoro-Pro, cis4Fluoro-Pro,” etc., is defined as follows:

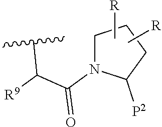

wherein, in this depiction, each R is independently hydrogen or a substituent.

In yet another embodiment, the compound of Formula I is selected from the group consisting of a compound of Table 1. In yet another embodiment, the compound of Formula VI is selected from the group consisting of a compound from Table 1.

In an aspect, provided herein is a cyclic peptide selected from the cyclic peptides of Table 1, or a pharmaceutically acceptable salt and/or solvate thereof.

TABLE 1

| Ex | Structure |
|---|---|
| 1 | 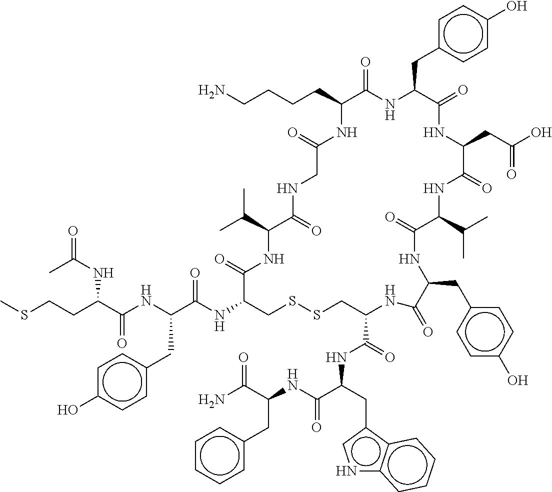 |
| 2 | 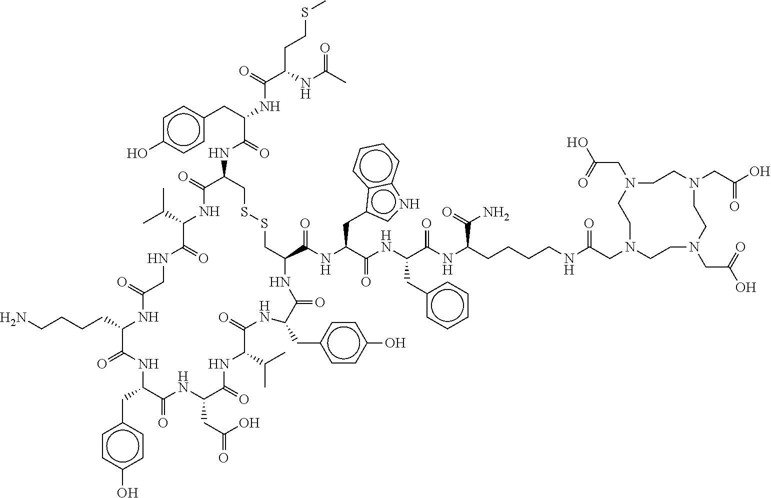 |
| 3 | 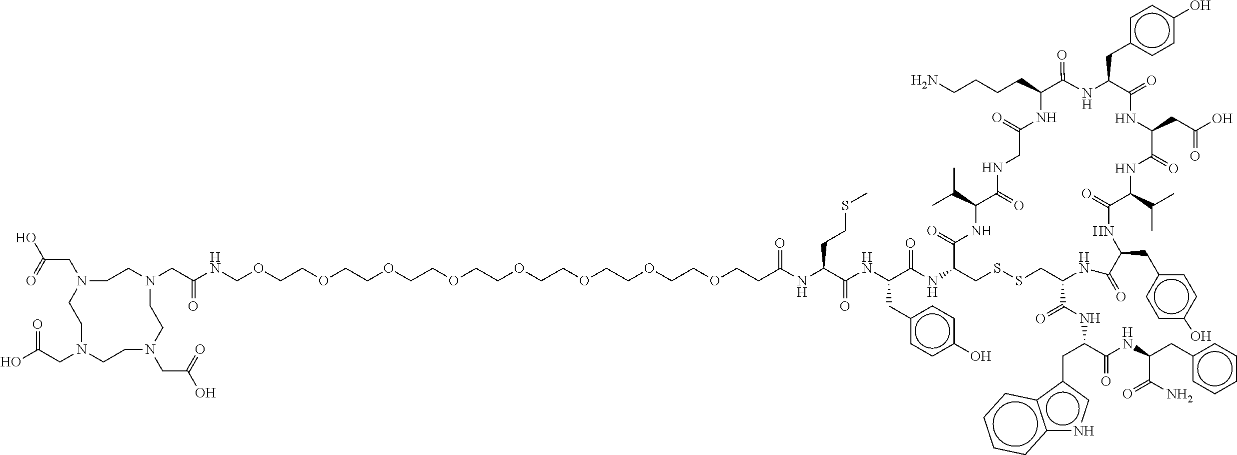 |
| 4 | 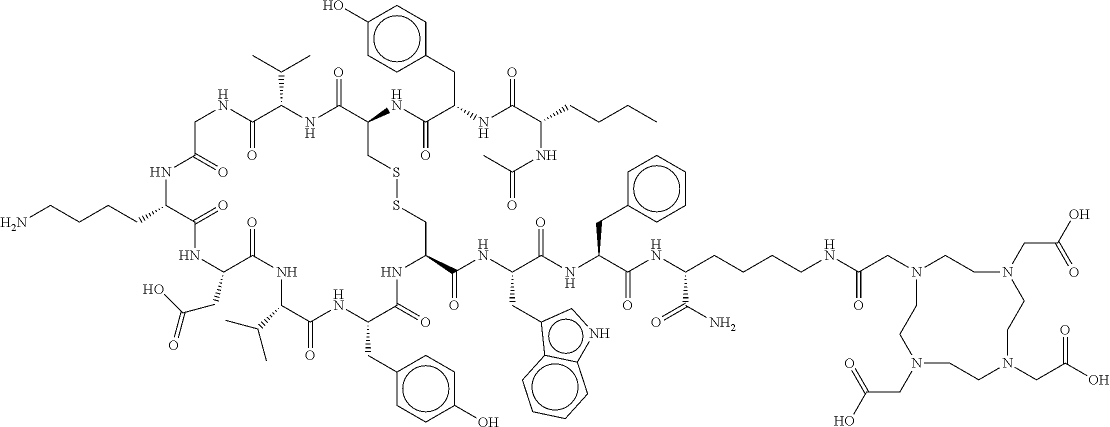 |
| 5 | 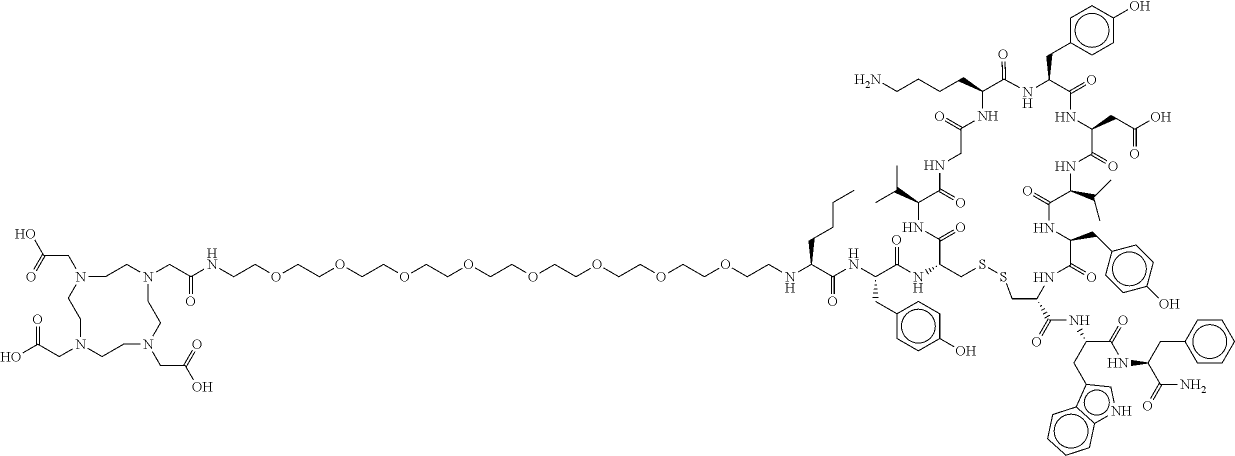 |
| 6 | 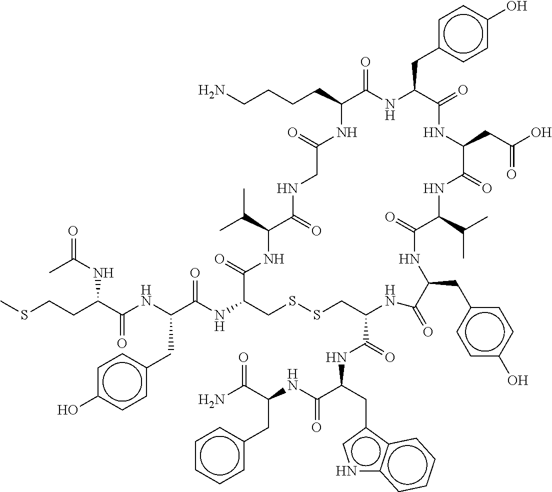 |
| 7 | 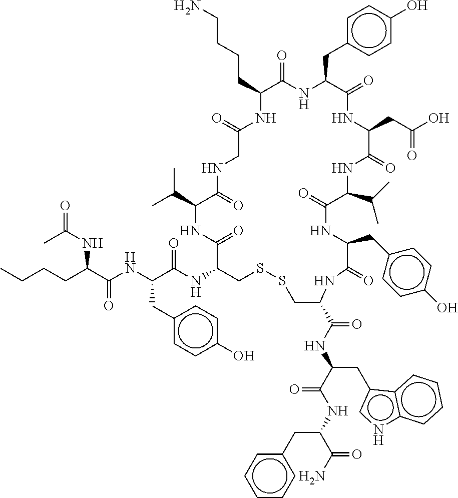 |
| 8 | 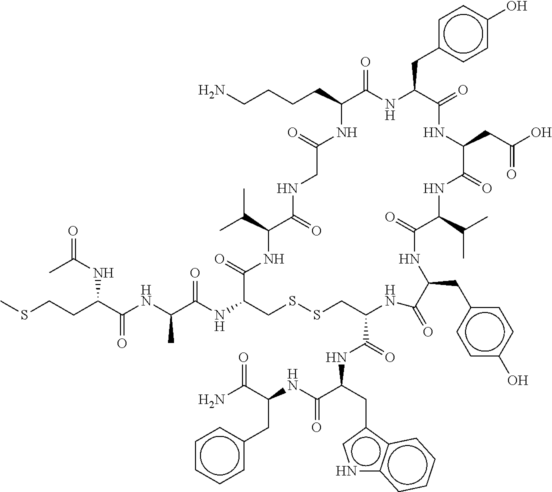 |
| 9 | 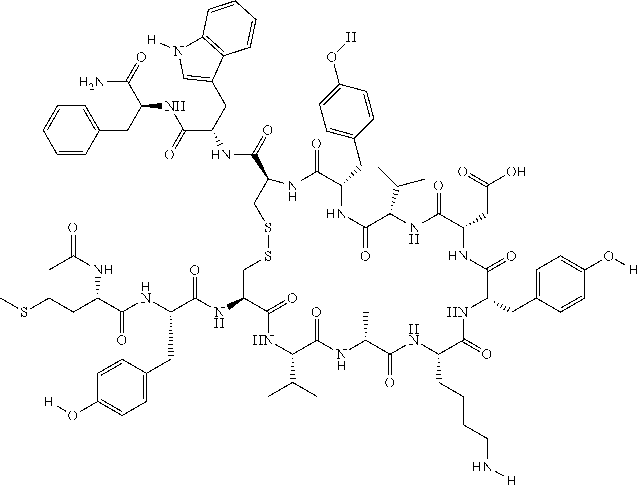 [SMILES: CSCC[C@H](NC(C)=O)C(=O)N[C@@H](Cc1ccc(O)cc1)C(=O)N[C@H]1CSSC[C@@H](C(=O)N[C@@H](Cc2c[nH]c3ccccc23)C(=O)N[C@@H](Cc2ccccc2)C(N)=O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCN)NC(=O)[C@@H](C)NC(=O)[C@H](C(C)C)NC1=O] |
| 10 | 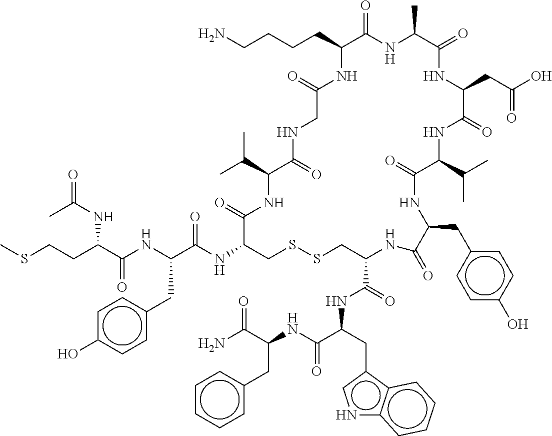 |
| 11 | 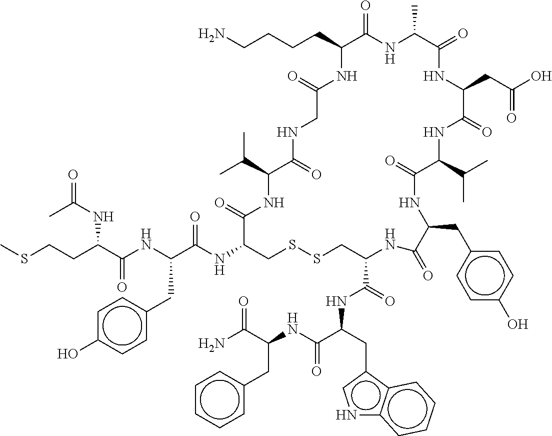 |
| 12 | 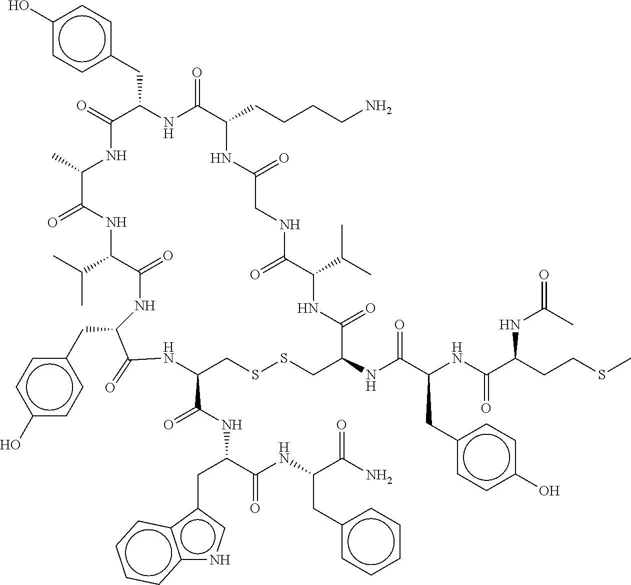 |
| 13 | 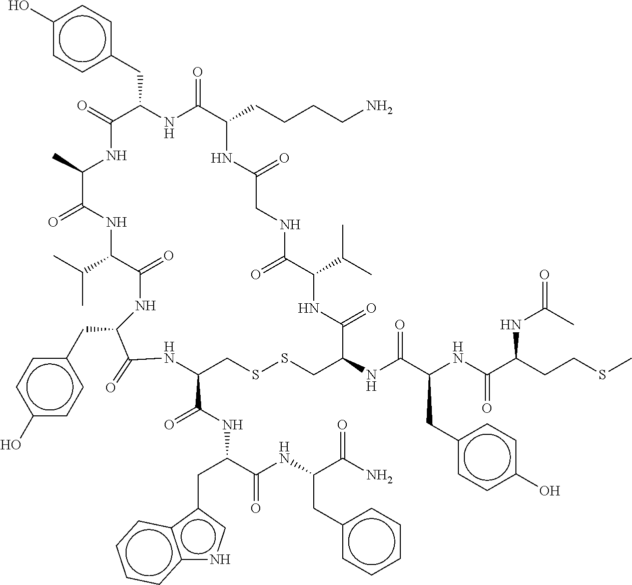 |
| 14 | 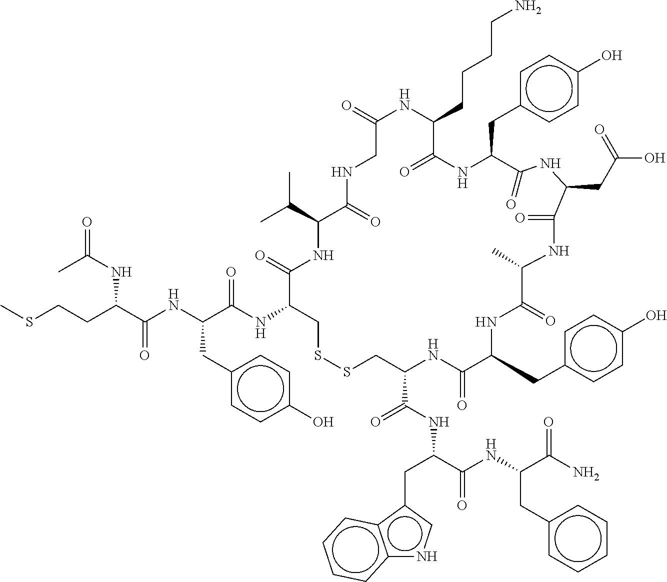 |
| 15 | 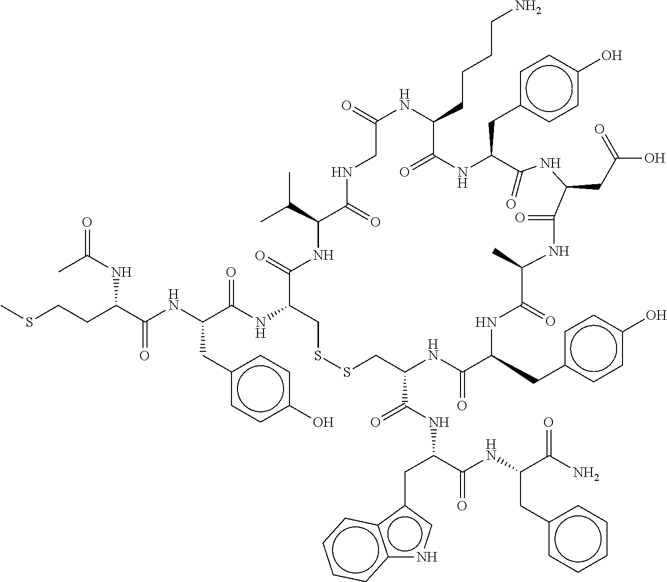 |
| 16 | 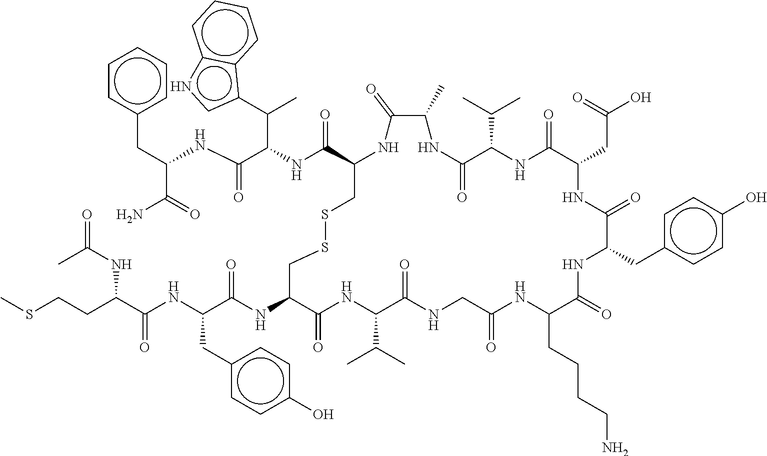 |
| 17 | 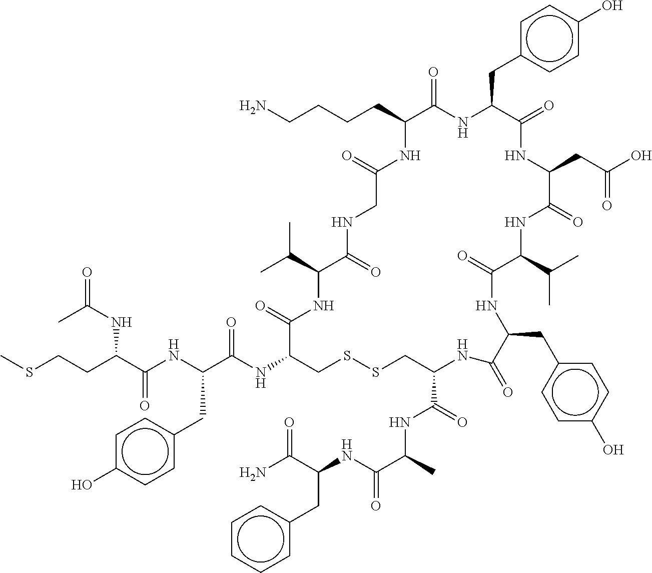 |
| 18 | 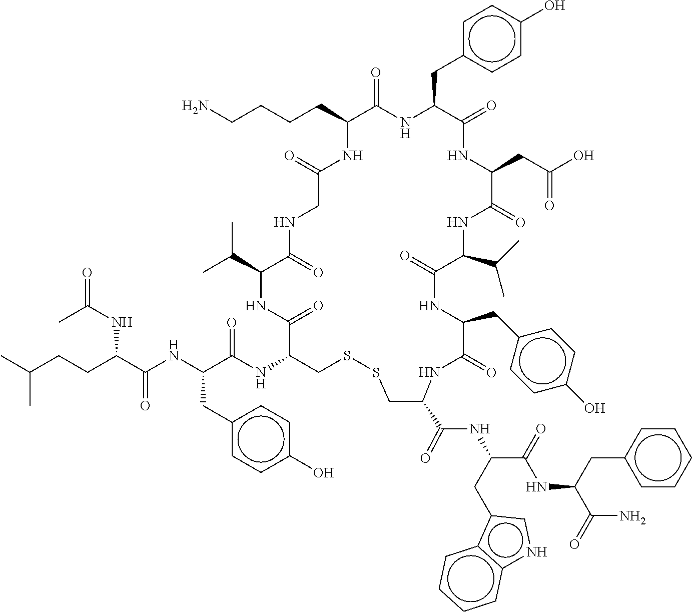 |
| 19 | 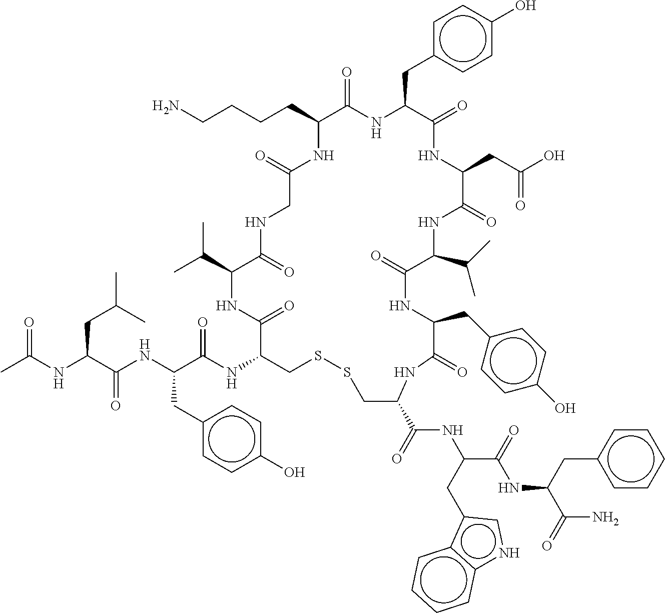 |
| 20 | 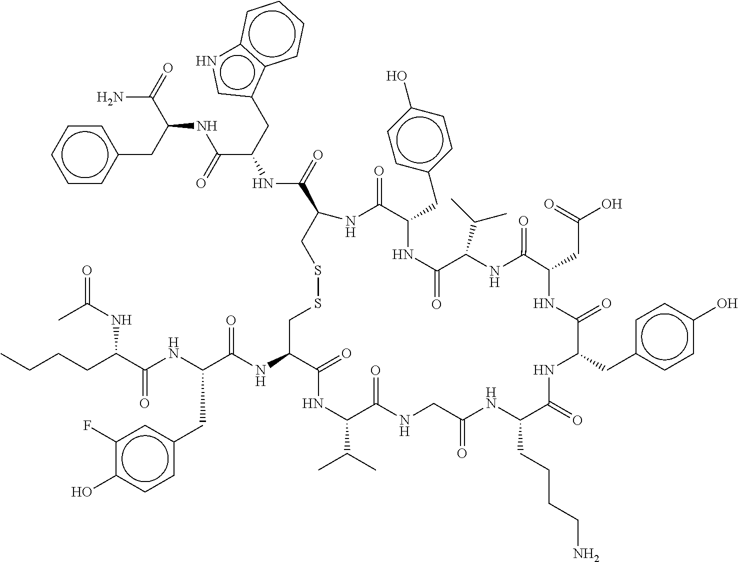 |
| 21 | 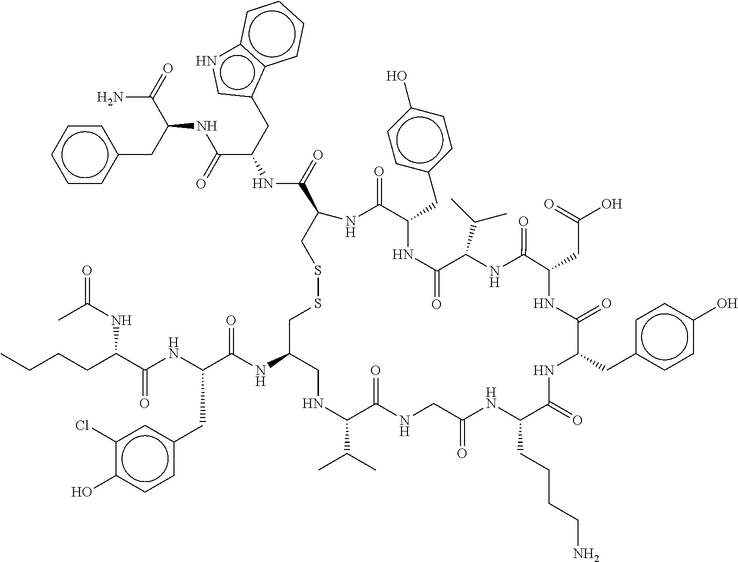 |
| 22 | 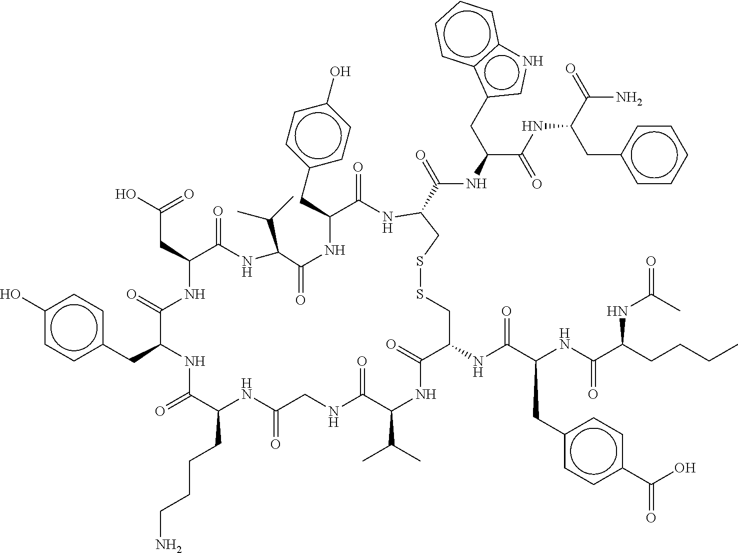 |
| 23 | 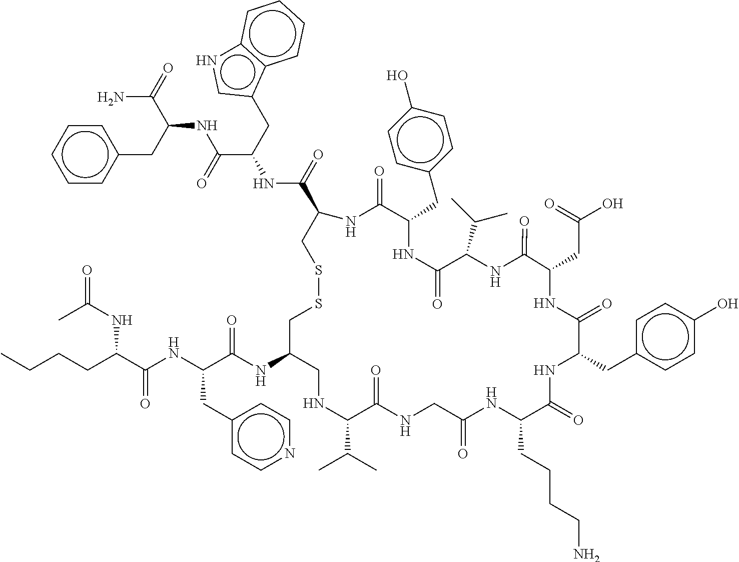 |
| 24 | 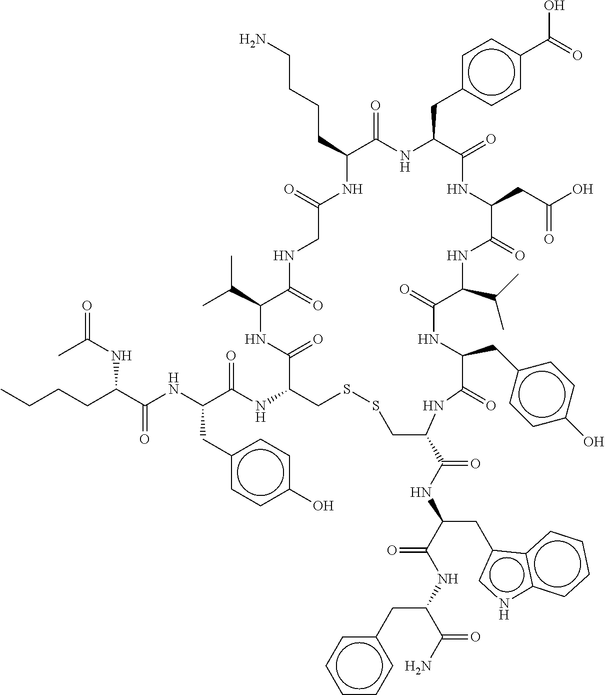 |
| 25 | 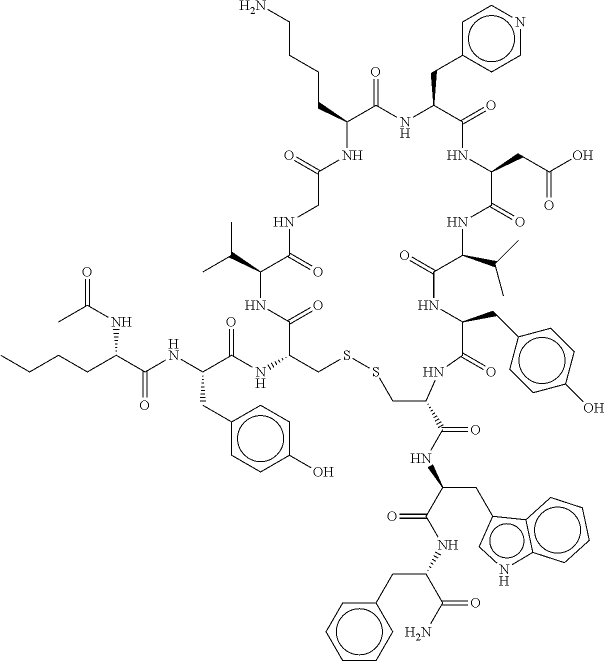 |
| 26 | 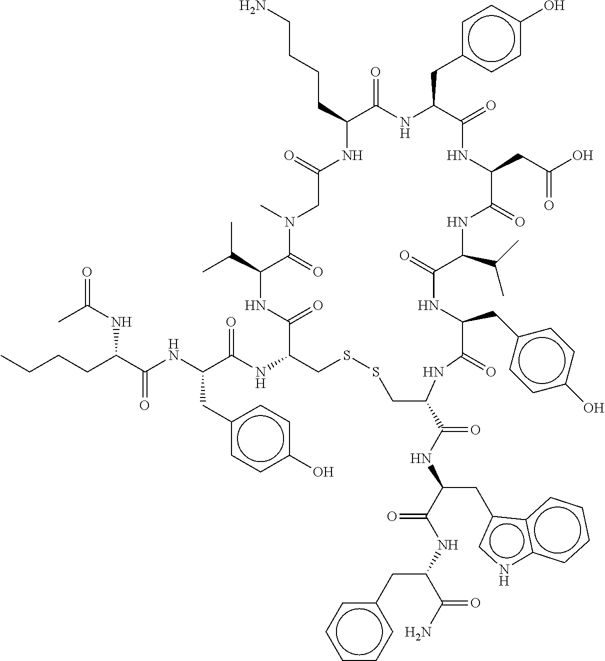 |
| 27 | 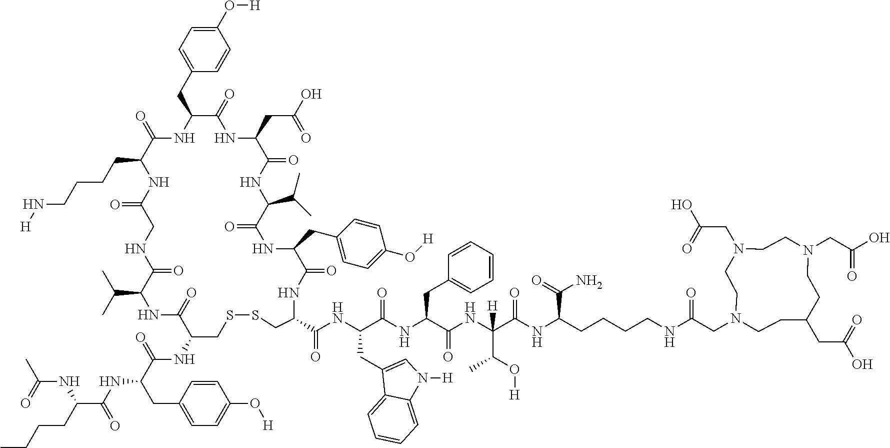 [SMILES: CCCC[C@H](NC(C)=O)C(=O)N[C@@H](Cc1ccc(O)cc1)C(=O)N[C@H]1CSSC[C@@H](C(=O)N[C@@H](Cc2c[nH]c3ccccc23)C(=O)N[C@@H](Cc2ccccc2)C(=O)N[C@H](C(=O)N[C@H](CCCCNC(=O)CN2CCC(CC(=O)O)CCN(CC(=O)O)CCN(CC(=O)O)CC2)C(N)=O)[C@@H](C)O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCN)NC(=O)CNC(=O)[C@H](C(C)C)NC1=O] |
| 28 | 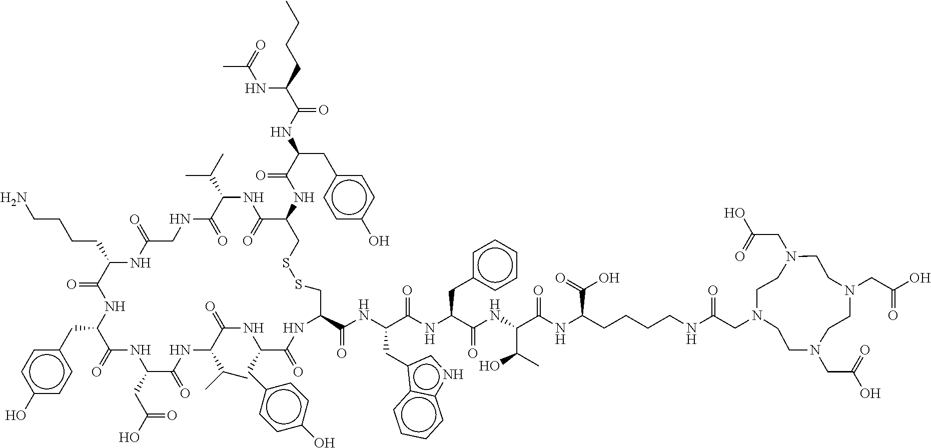 |
| 29 | 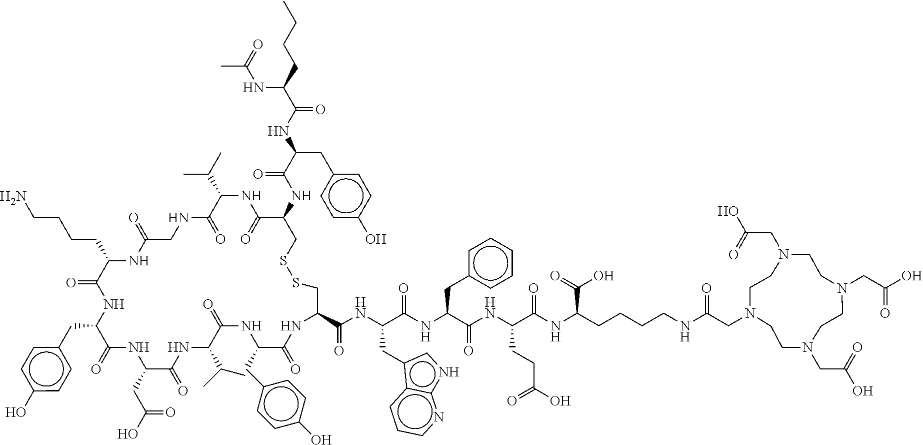 |
| 30 | 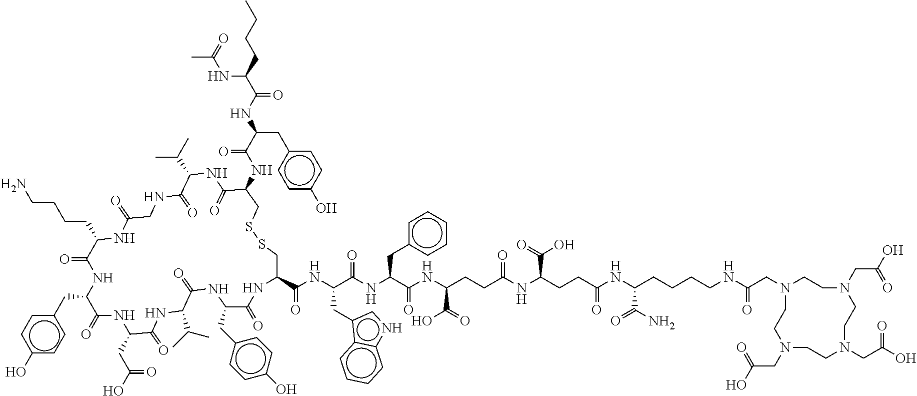 |
| 31 | 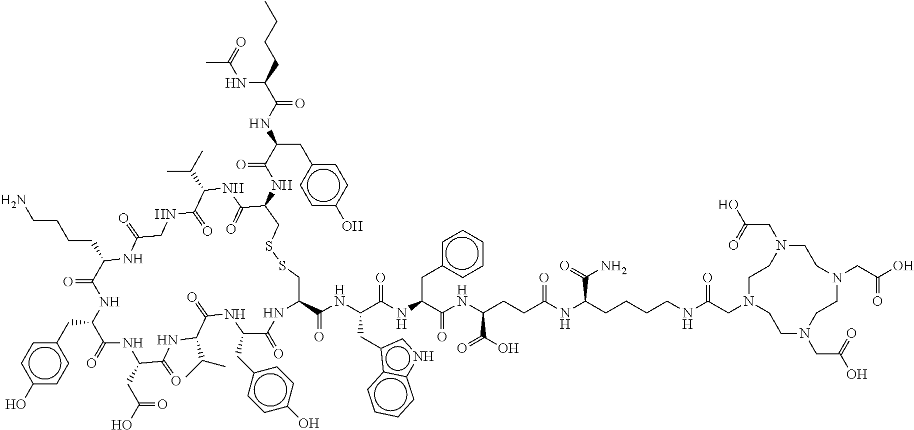 |
| 32 | 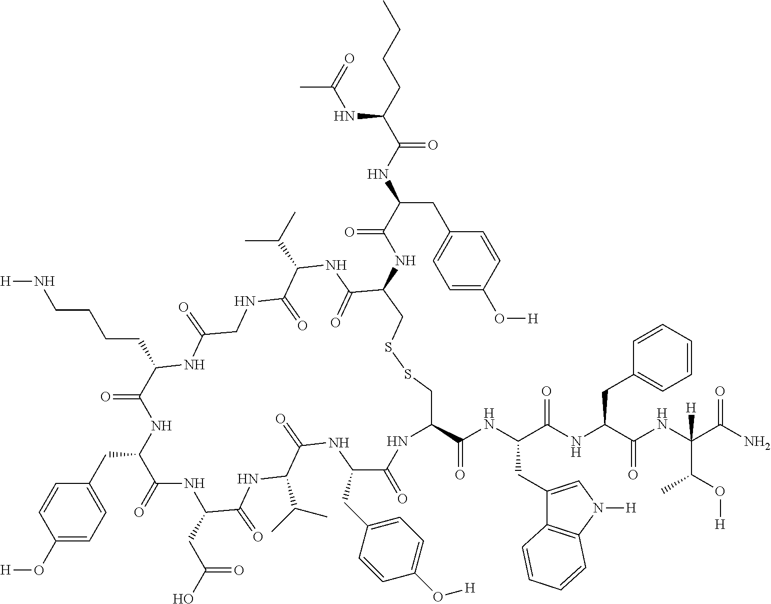 [SMILES: CCCC[C@H](NC(C)=O)C(=O)N[C@@H](Cc1ccc(O)cc1)C(=O)N[C@H]1CSSC[C@@H](C(=O)N[C@@H](Cc2c[nH]c3ccccc23)C(=O)N[C@@H](Cc2ccccc2)C(=O)N[C@H](C(N)=O)[C@@H](C)O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCN)NC(=O)CNC(=O)[C@H](C(C)C)NC1=O] |
| 33 | 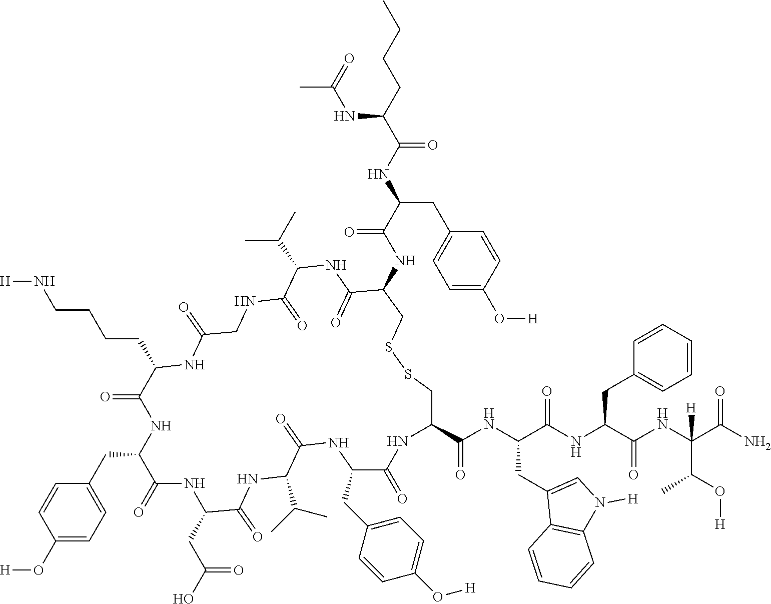 [SMILES: CCCC[C@H](NC(C)=O)C(=O)N[C@@H](Cc1ccc(O)cc1)C(=O)N[C@H]1CSSC[C@@H](C(=O)N[C@@H](Cc2c[nH]c3ccccc23)C(=O)N[C@@H](Cc2ccccc2)C(=O)N[C@H](C(N)=O)[C@@H](C)O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCN)NC(=O)CNC(=O)[C@H](C(C)C)NC1=O] |
| 34 | 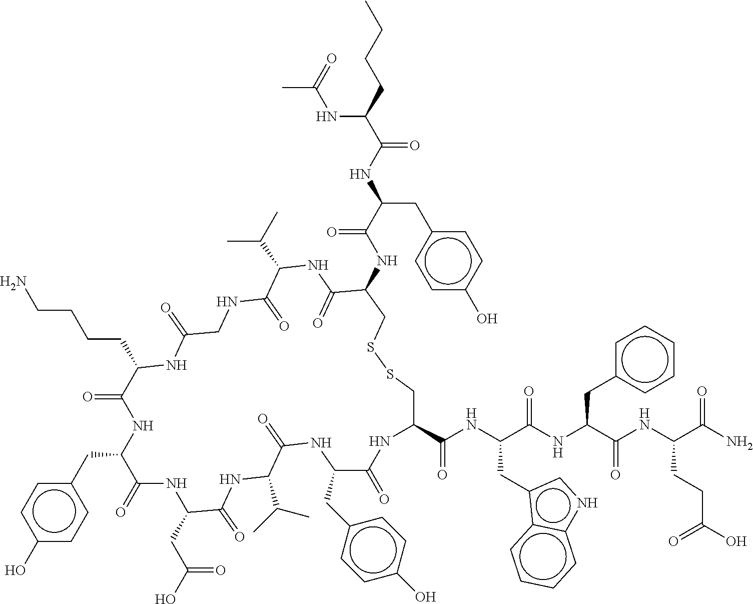 |
| 35 | 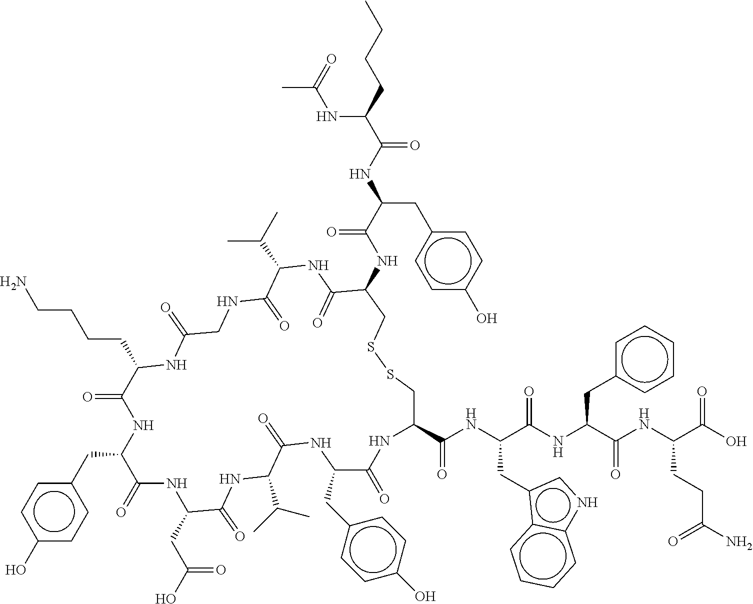 |
| 36 | 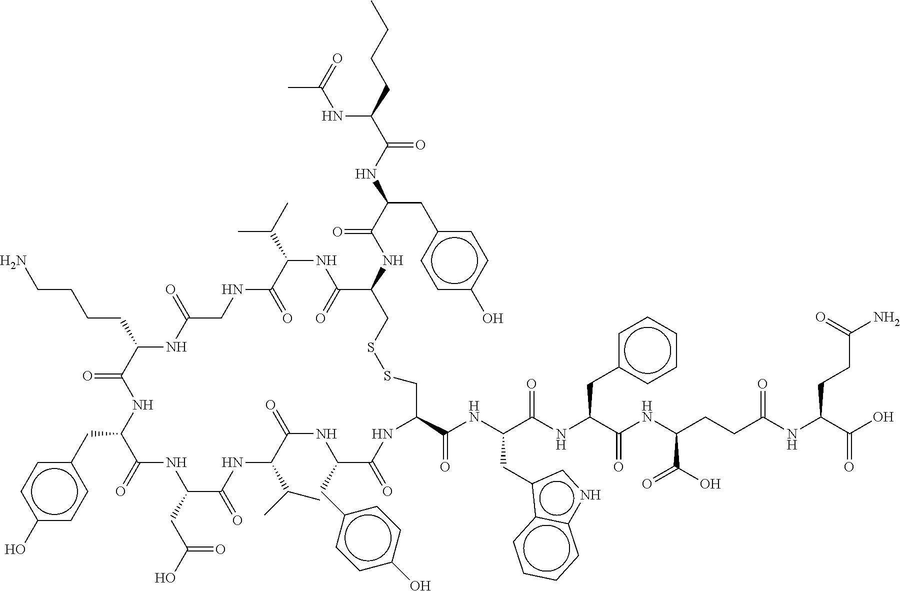 |
| 37 | 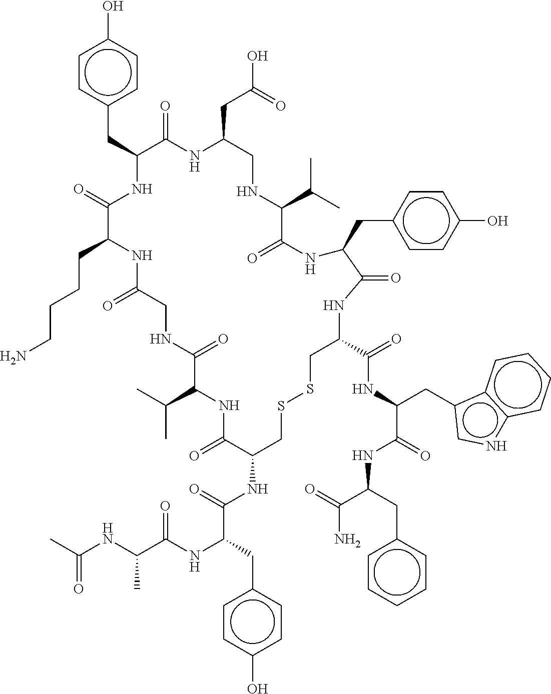 |
| 38 | 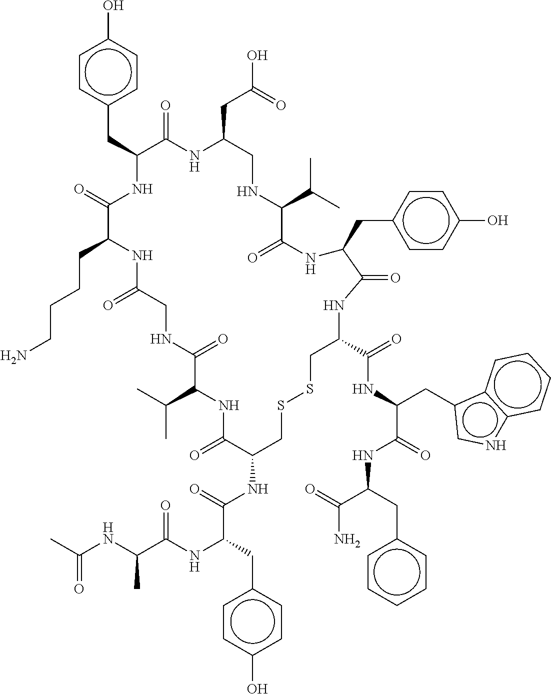 |
| 39 | 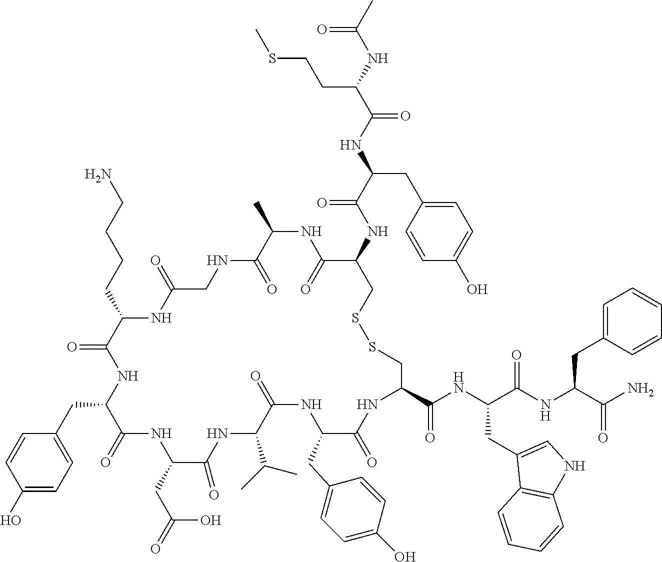 [SMILES: CSCC[C@H](NC(C)=O)C(=O)N[C@@H](Cc1ccc(O)cc1)C(=O)N[C@H]1CSSC[C@@H](C(=O)N[C@@H](Cc2c[nH]c3ccccc23)C(=O)N[C@@H](Cc2ccccc2)C(N)=O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCN)NC(=O)CNC(=O)[C@@H](C)NC1=O] |
| 40 | 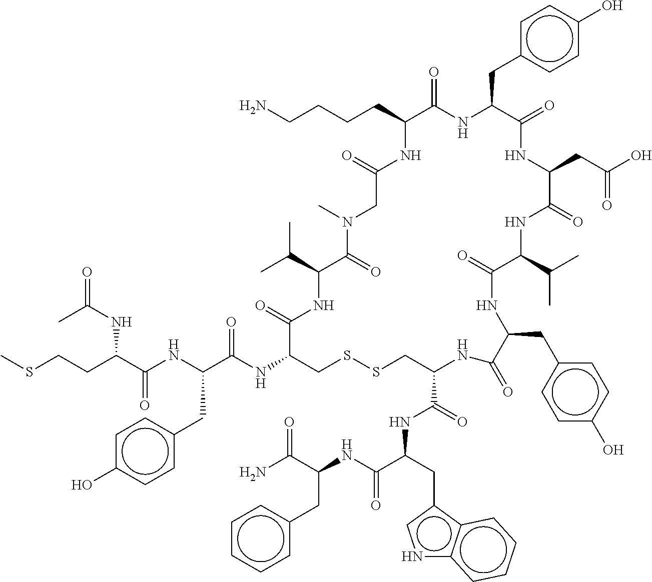 |
| 41 | 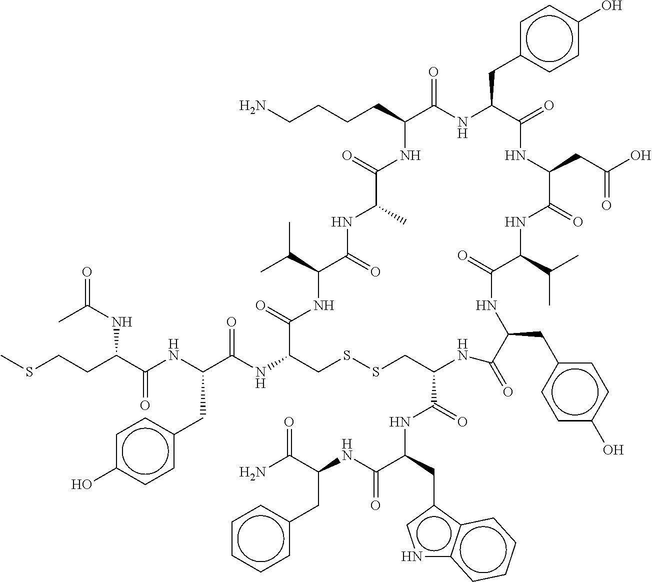 |
| 42 | 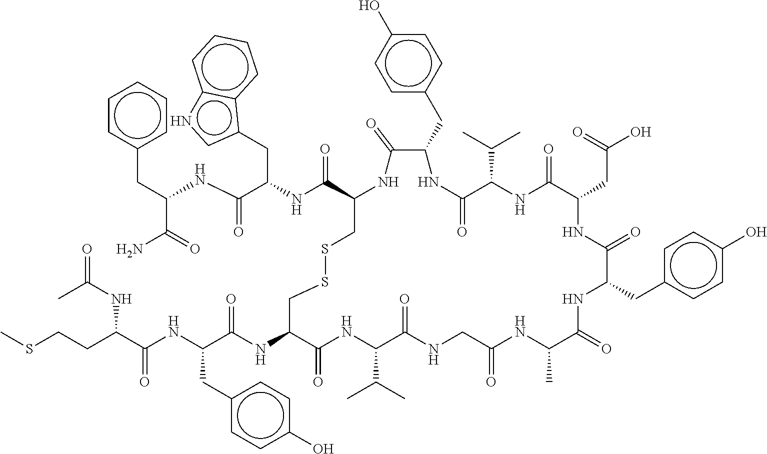 |
| 43 | 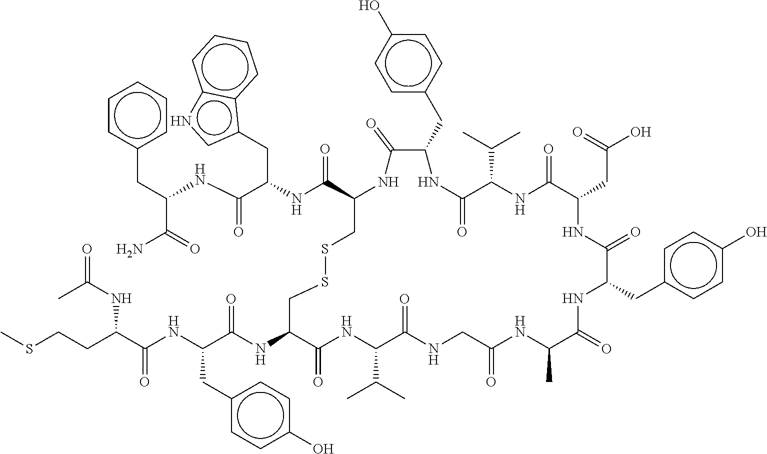 |
| 44 | 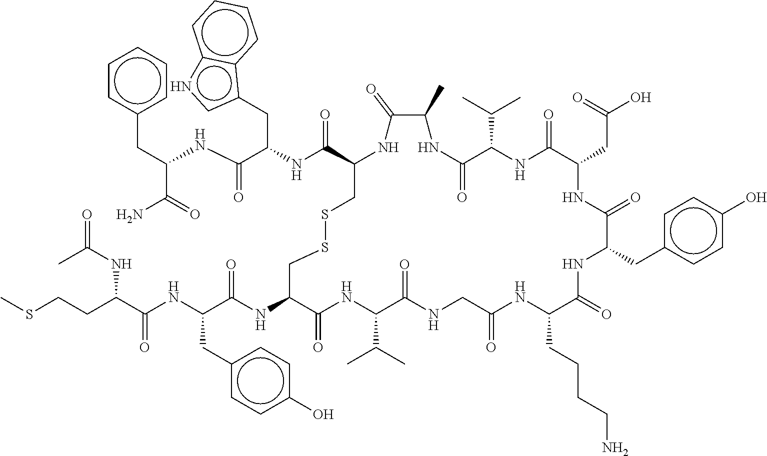 |
| 45 | 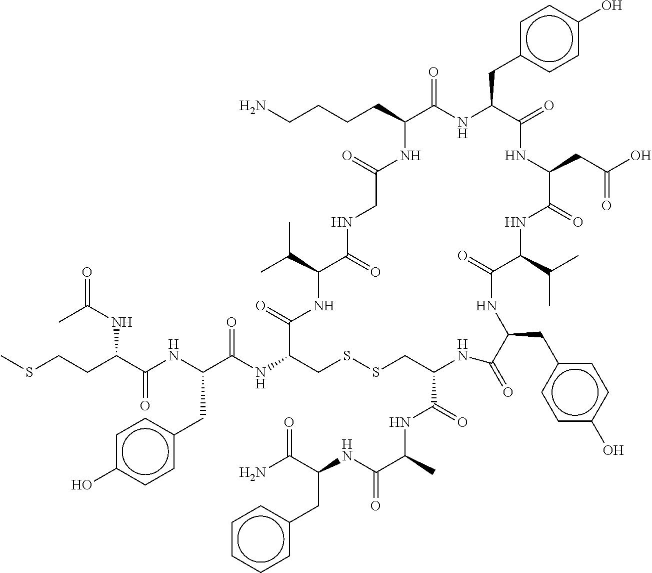 |
| 46 | 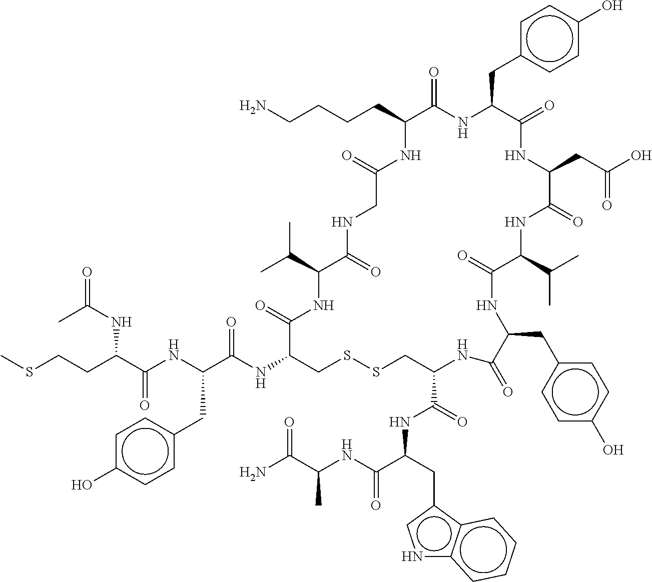 |
| 47 | 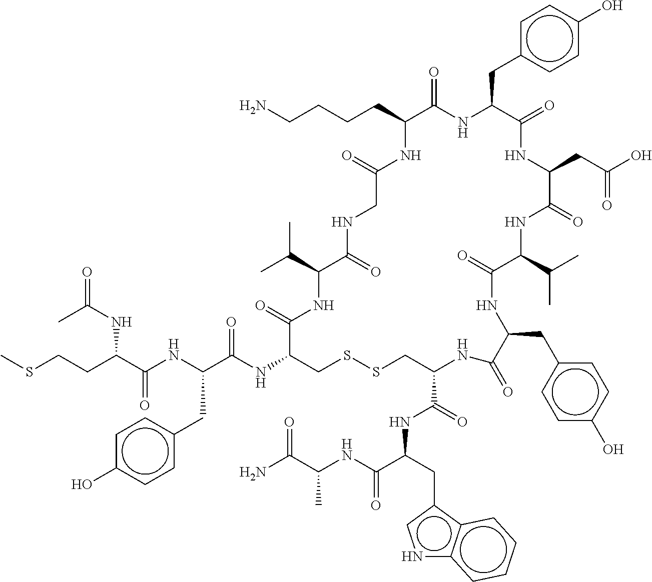 |
| 48 | 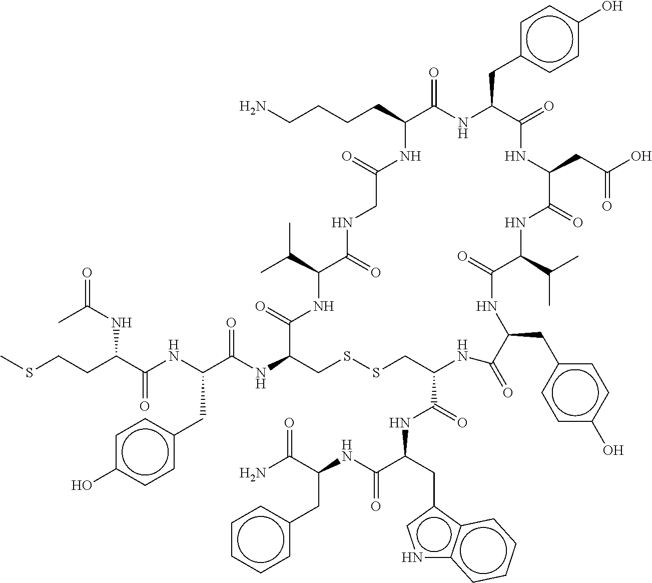 |
| 49 | 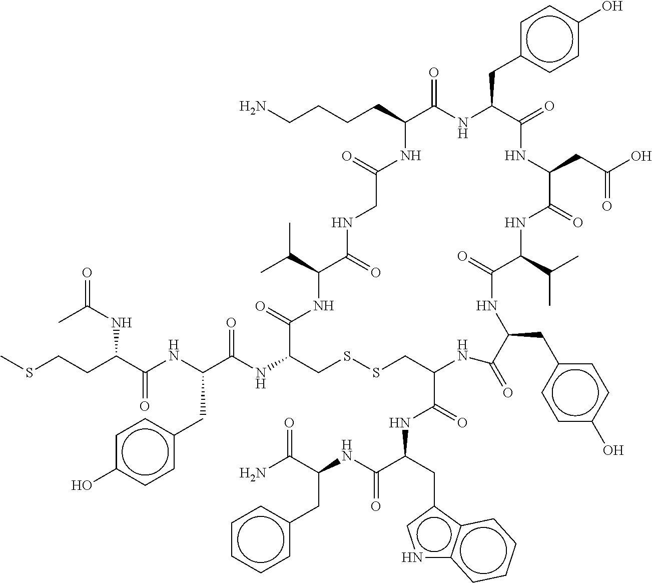 |
| 50 | 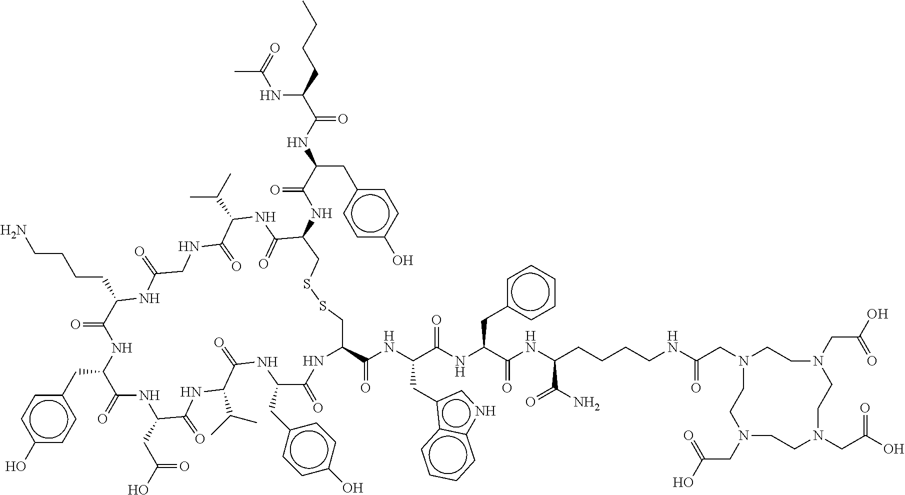 |
| 51 | 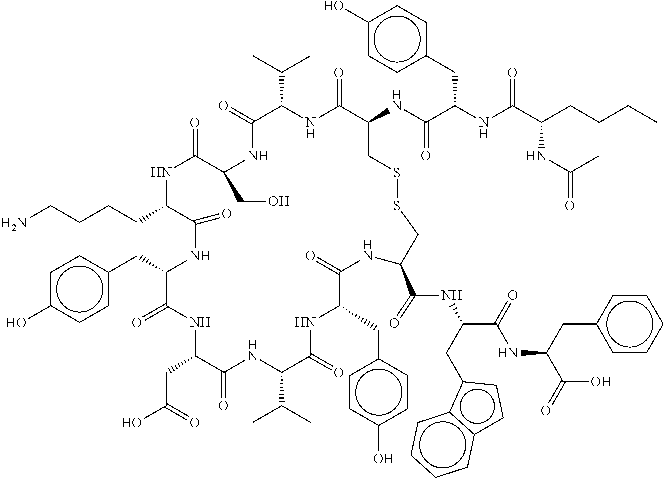 |
| 52 | 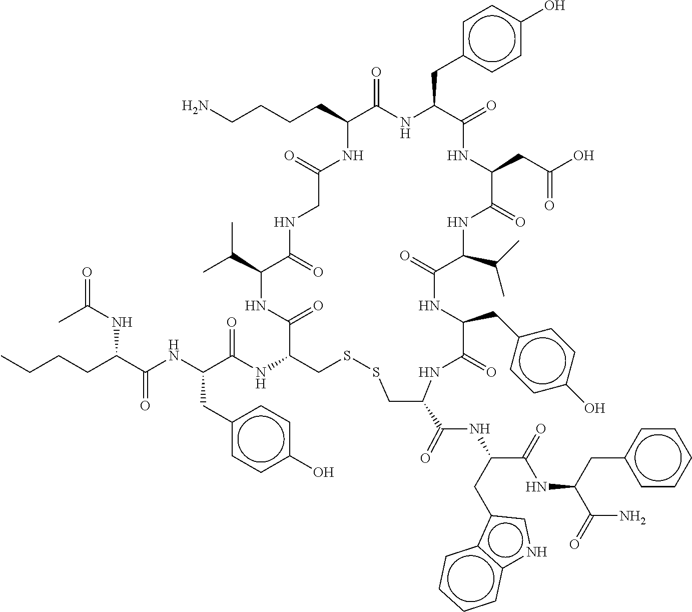 |
| 53 | 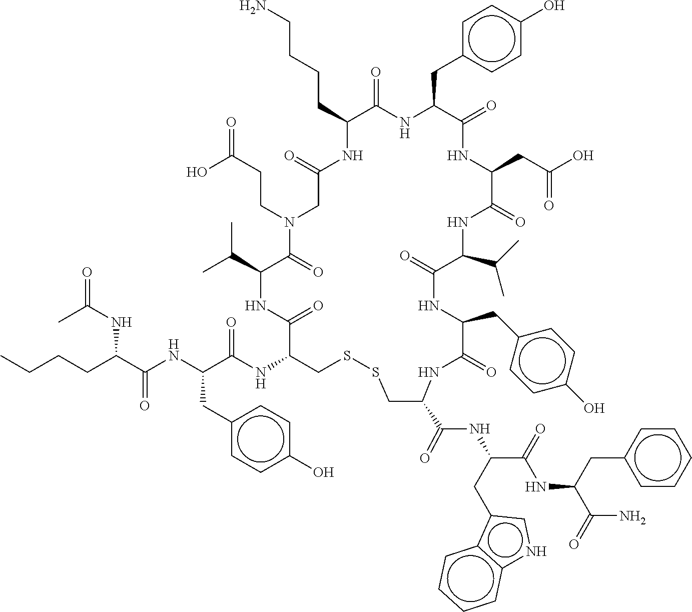 |
| 54 | 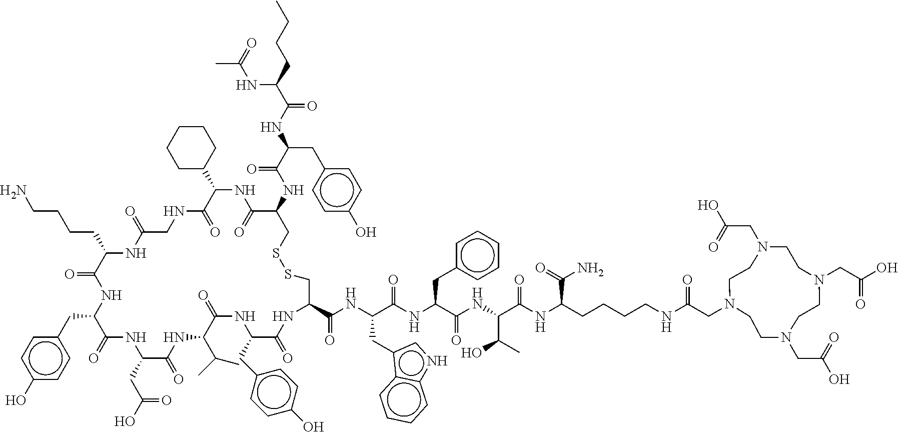 |
| 55 | 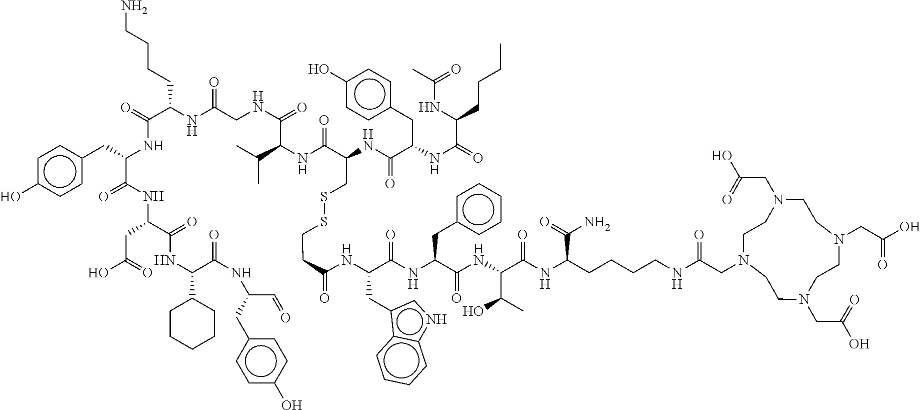 |
| 56 | 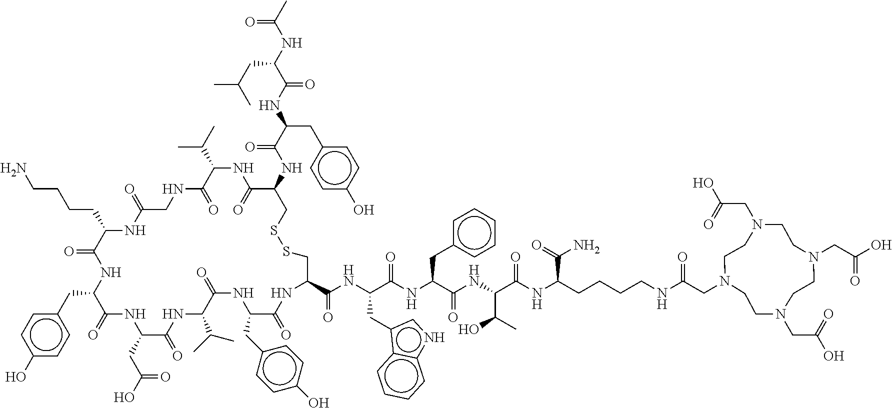 |
| 57 | 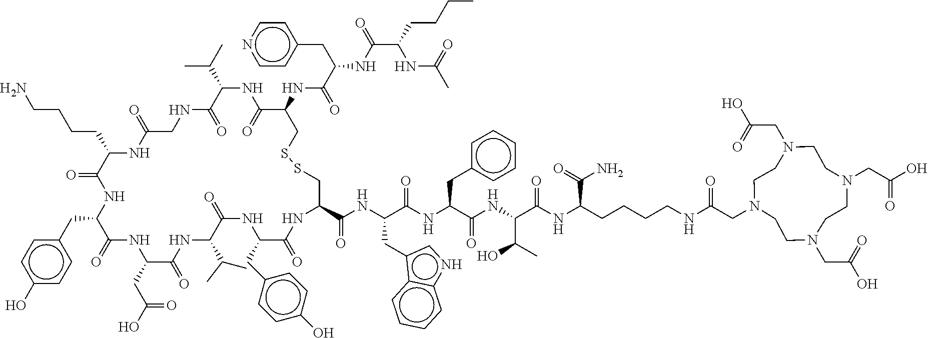 |
| 58 | 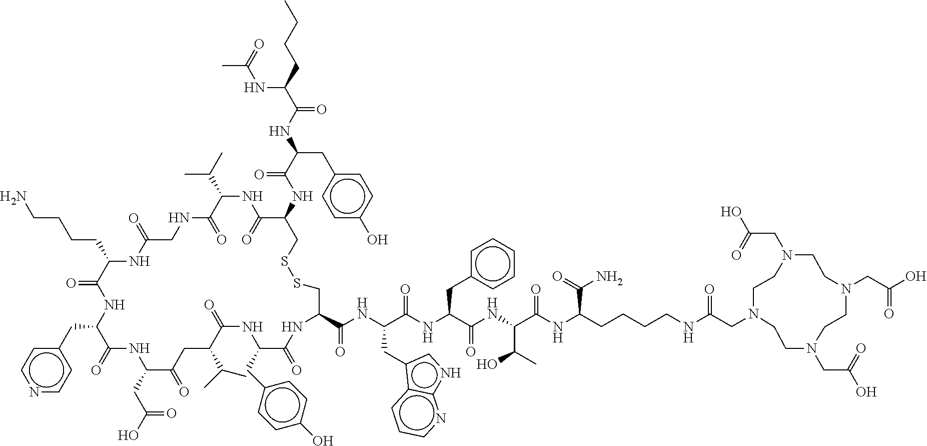 |
| 59 | 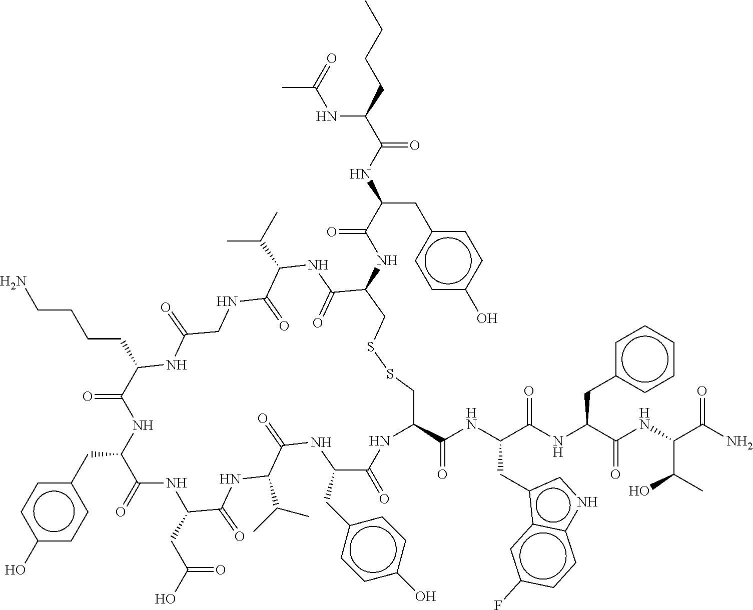 |
| 60 | 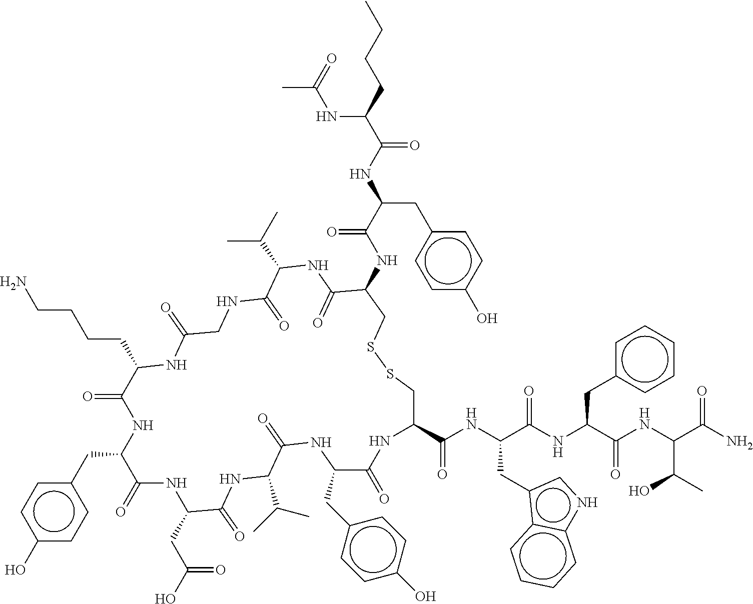 |
| 61 | 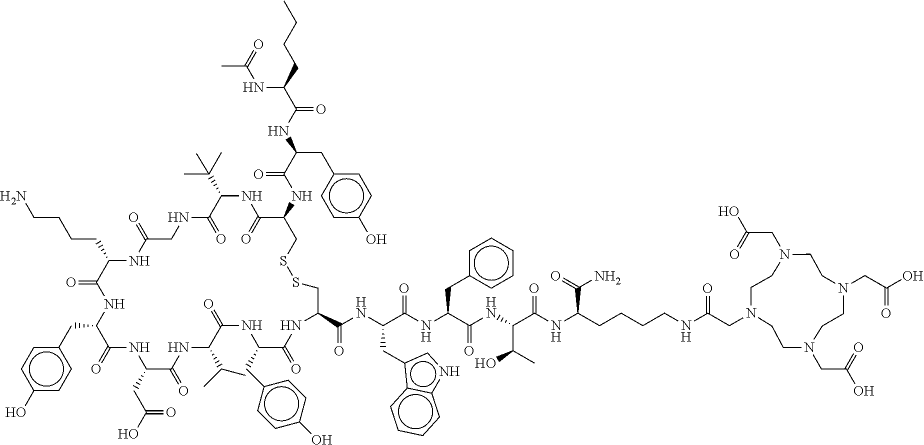 |
| 62 | 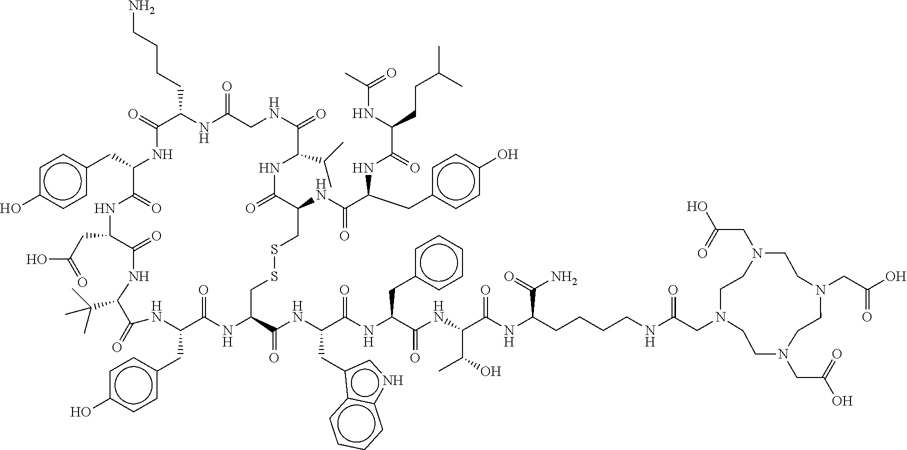 |
| 63 | 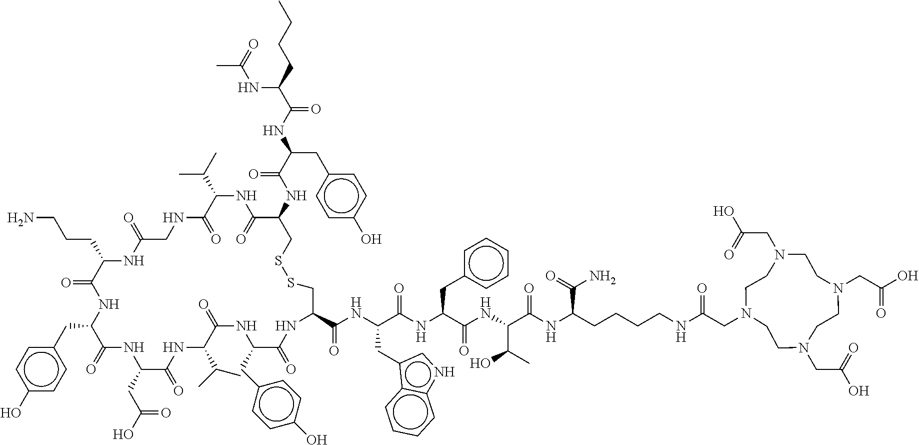 |
| 64 | 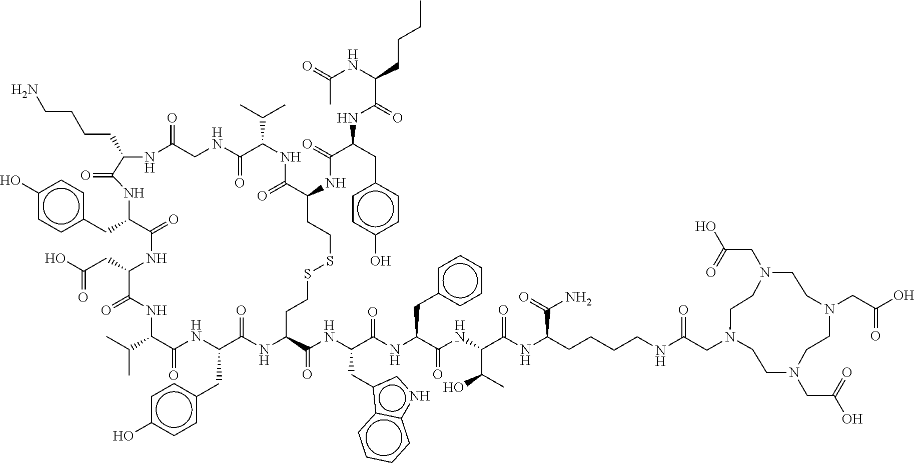 |
| 65 | 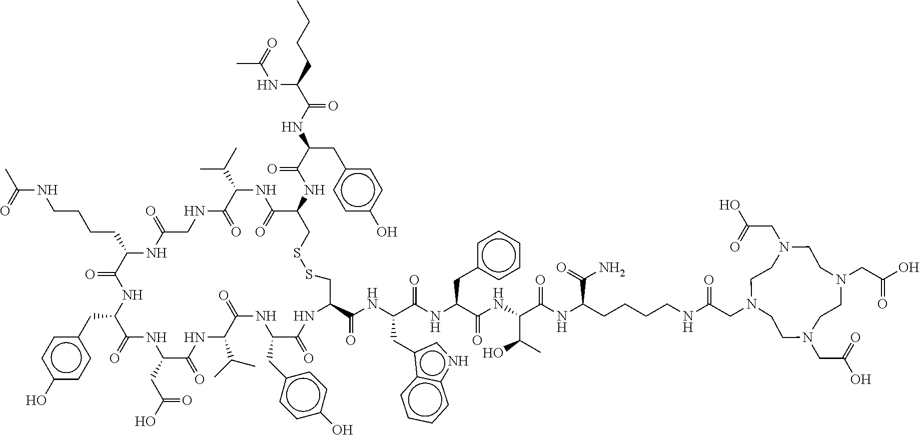 |
| 66 | 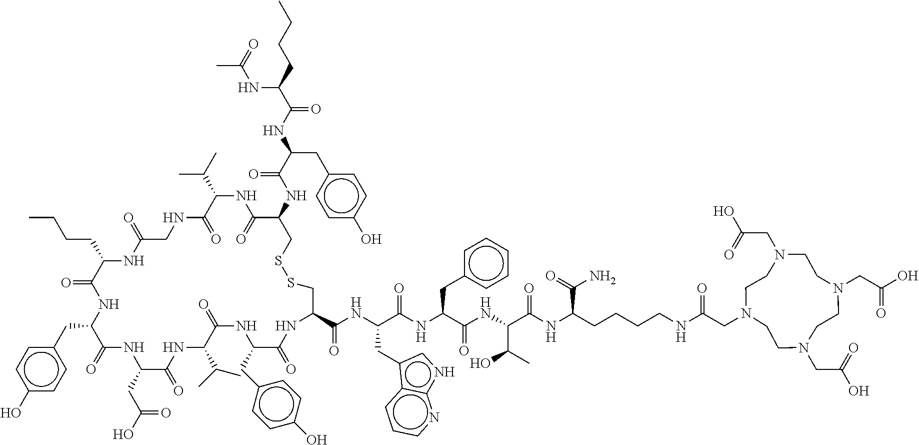 |
| 67 | 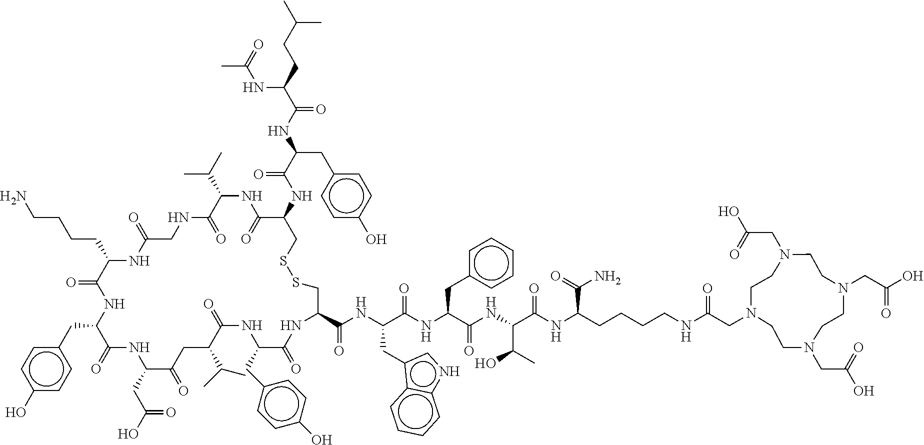 |
| 68 | 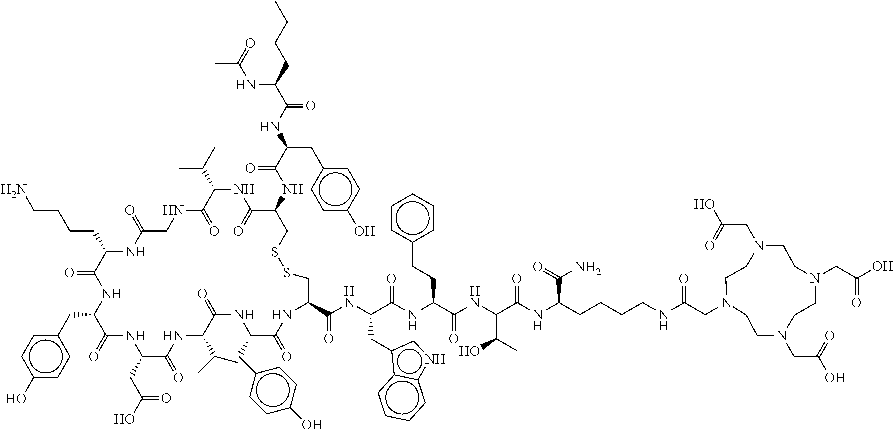 |
| 69 | 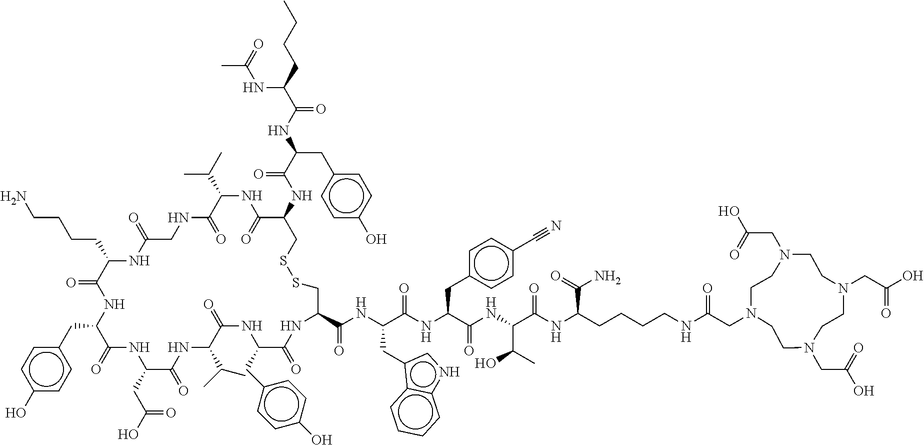 |
| 70 | 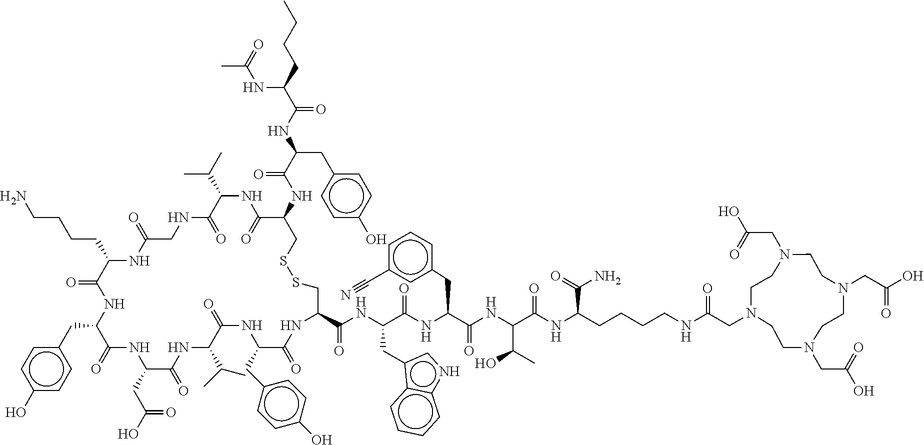 |
| 71 | 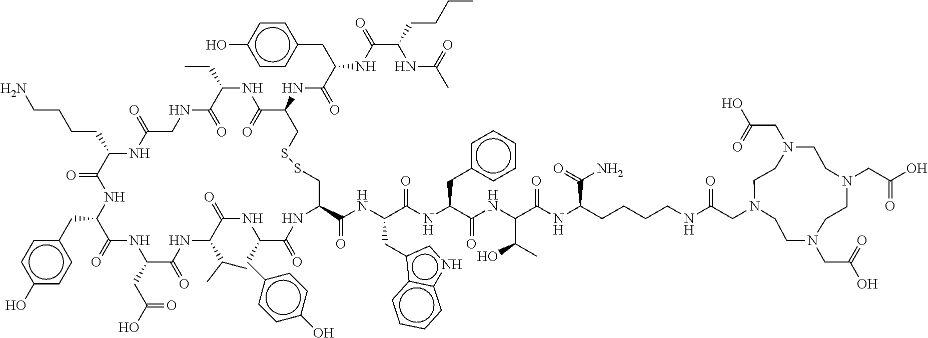 |
| 72 | 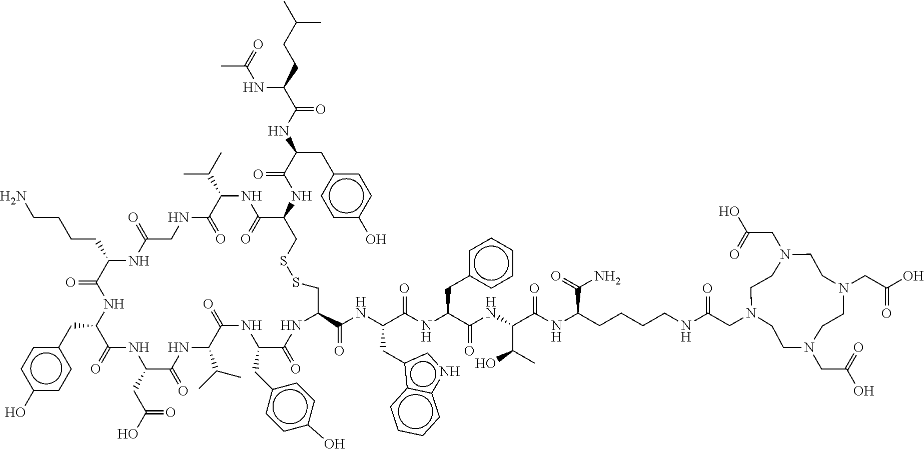 |
| 73 | 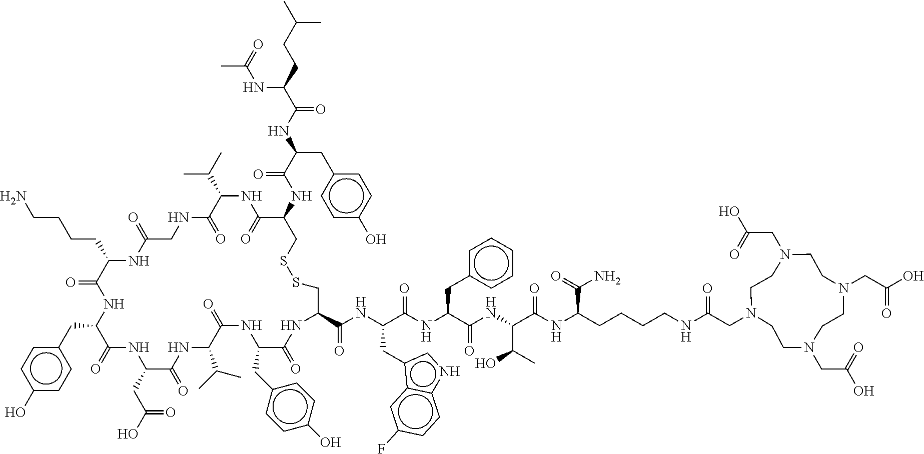 |
| 74 | 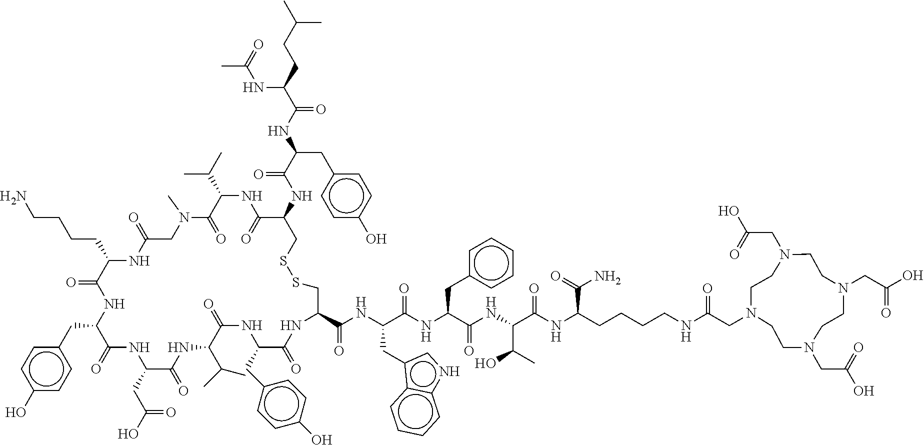 |
| 75 | 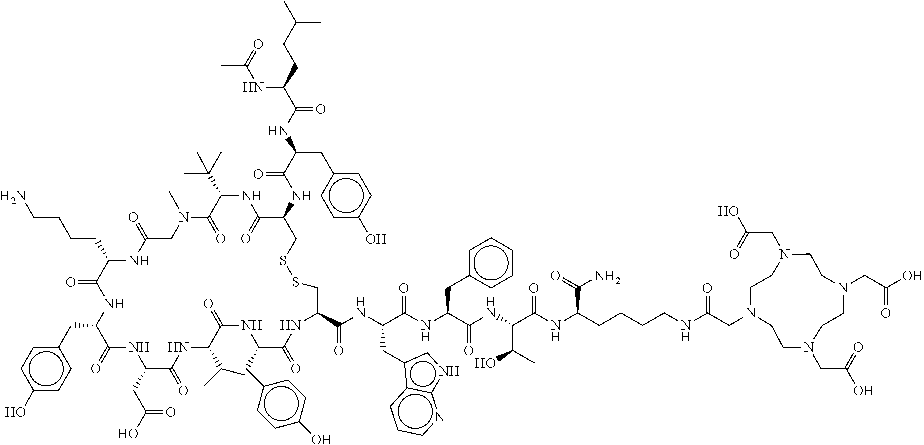 |
| 76 | 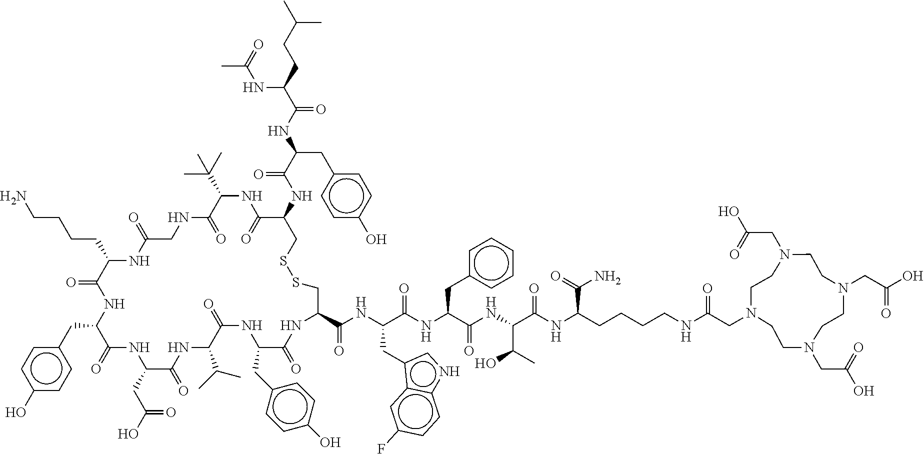 |
| 77 | 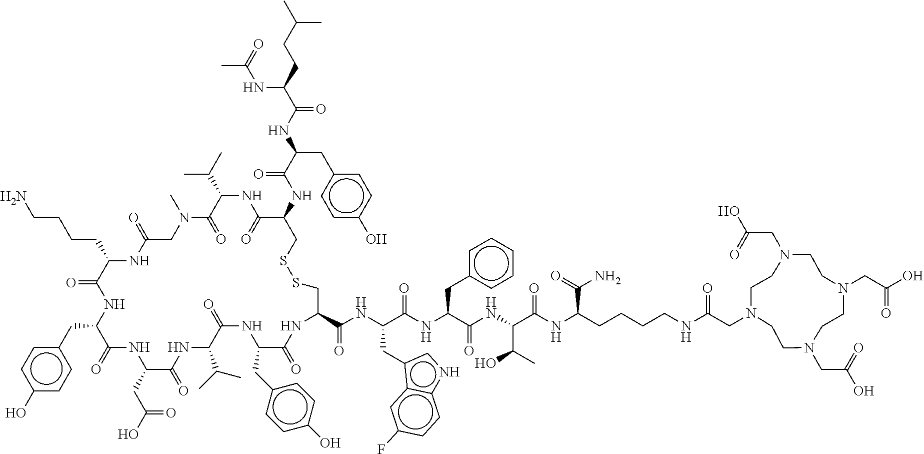 |
| 78 | 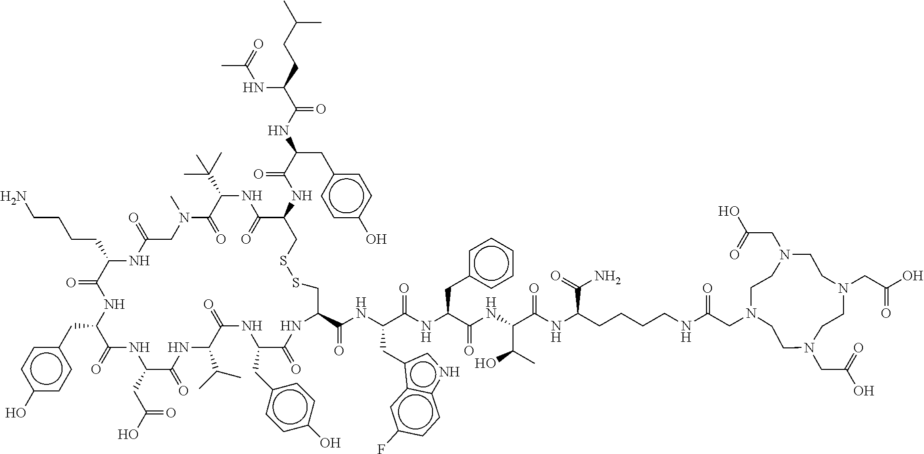 |
| 79 | 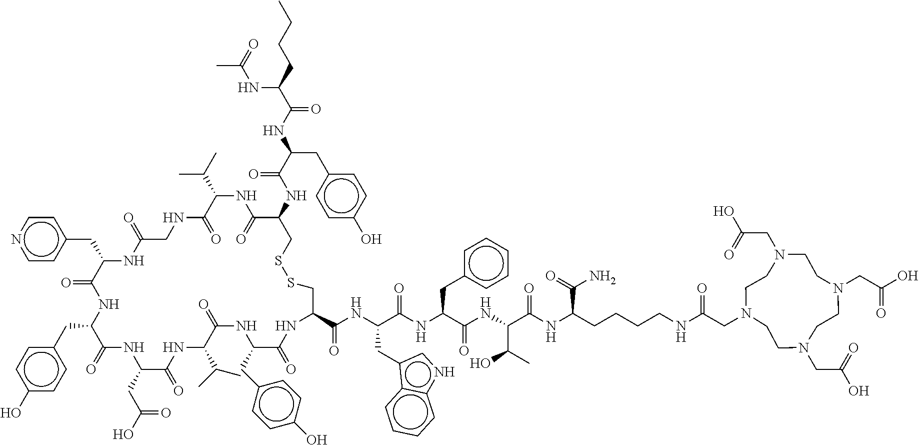 |
| 80 | 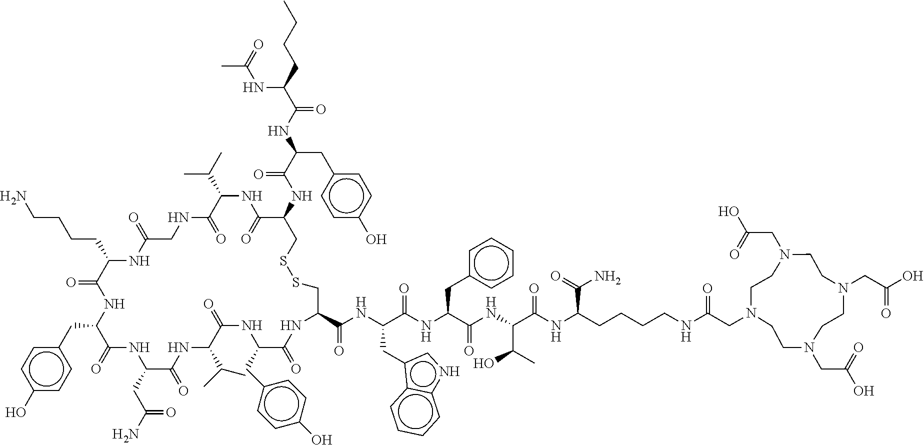 |
| 81 | 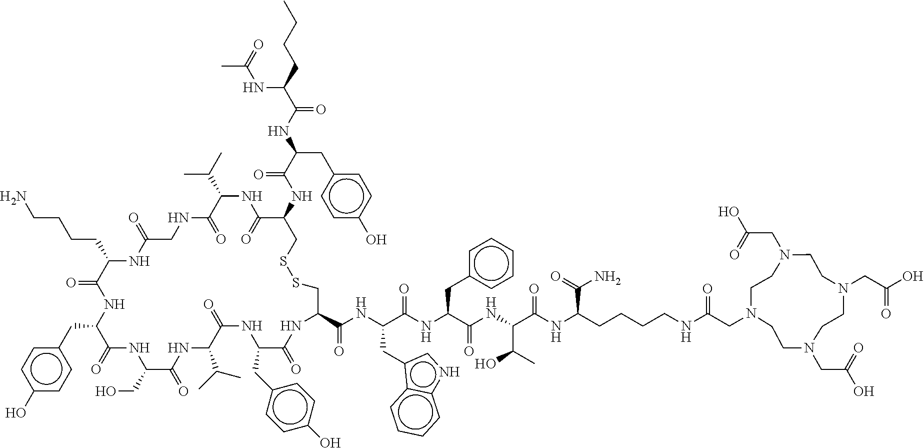 |
| 82 | 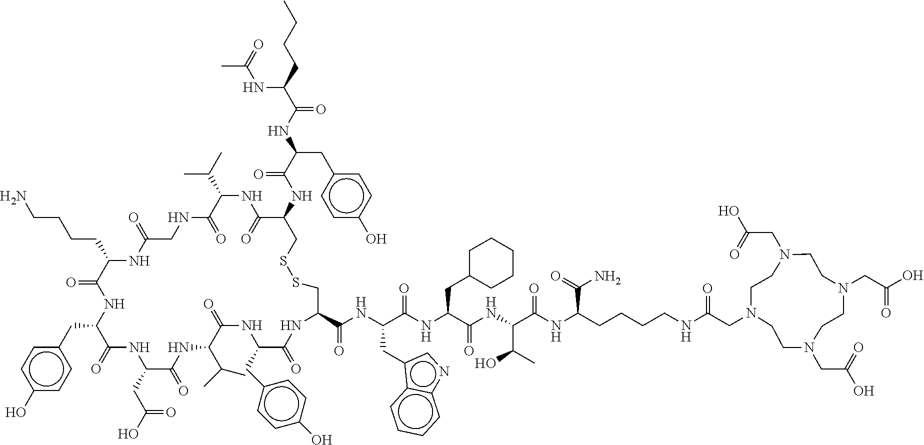 |
| 83 | 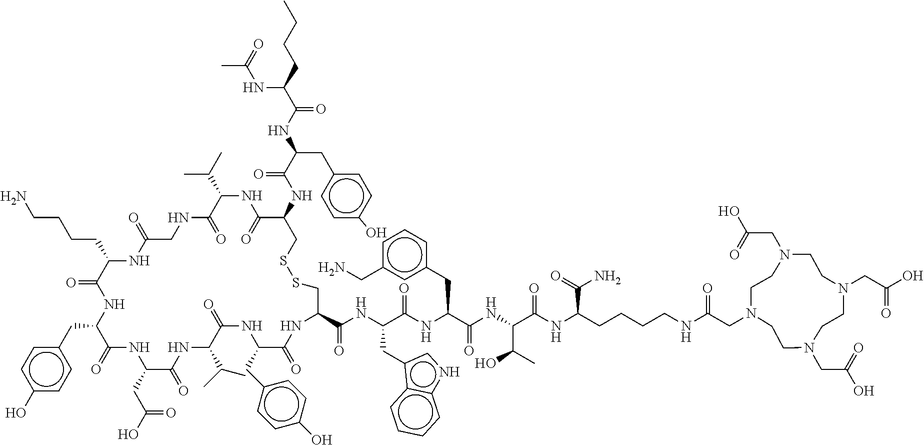 |
| 84 | 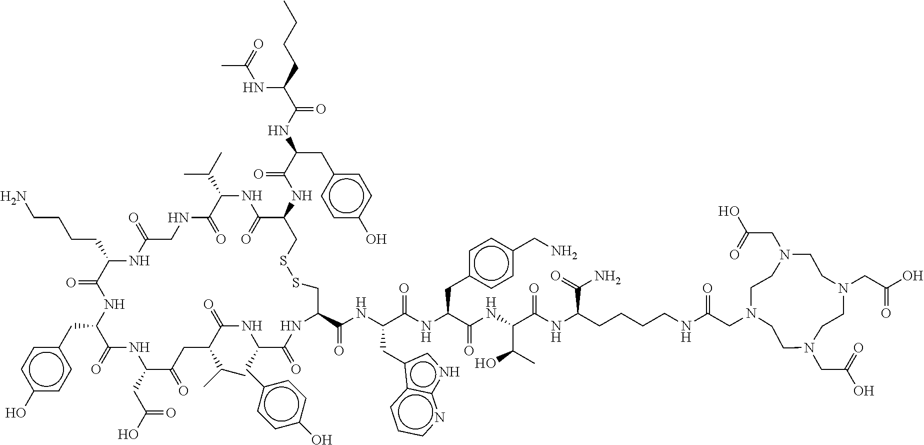 |
| 85 | 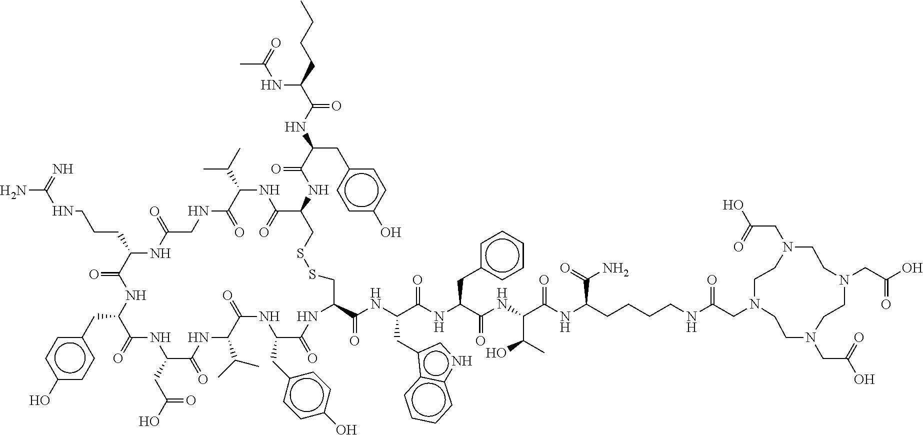 |
| 86 | 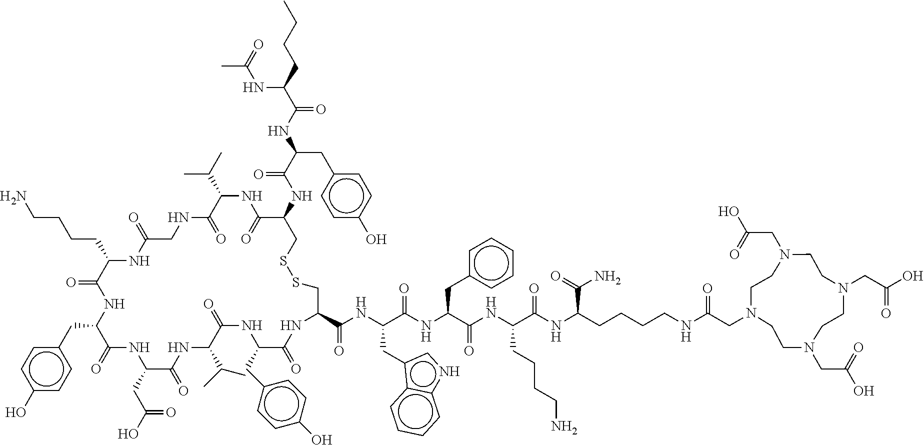 |
| 87 | 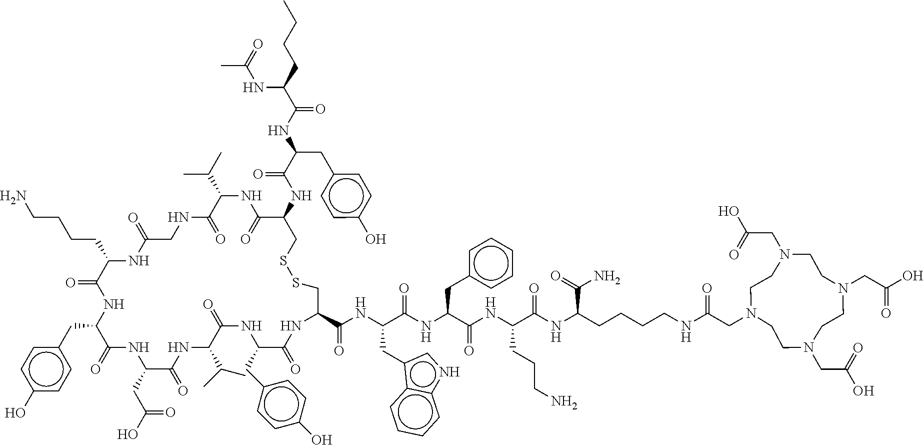 |
| 88 | 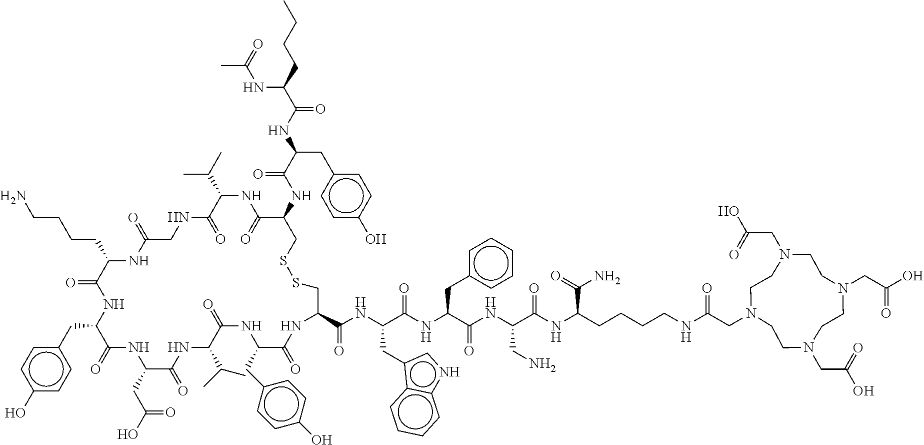 |
| 89 | 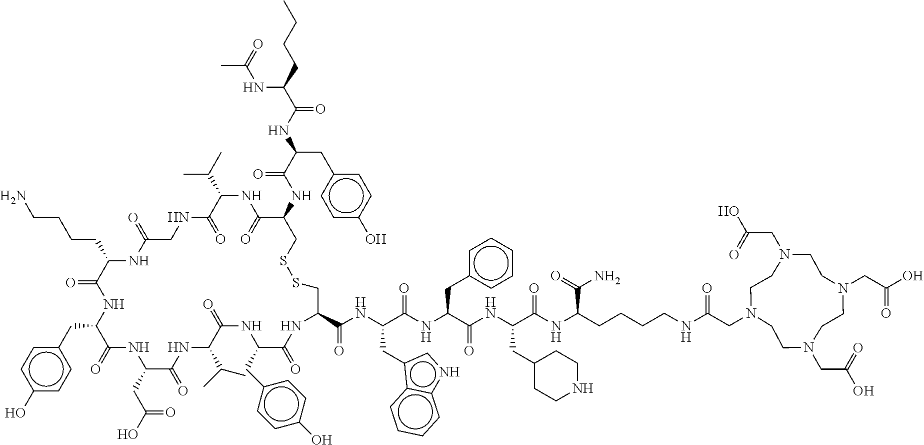 |
| 90 | 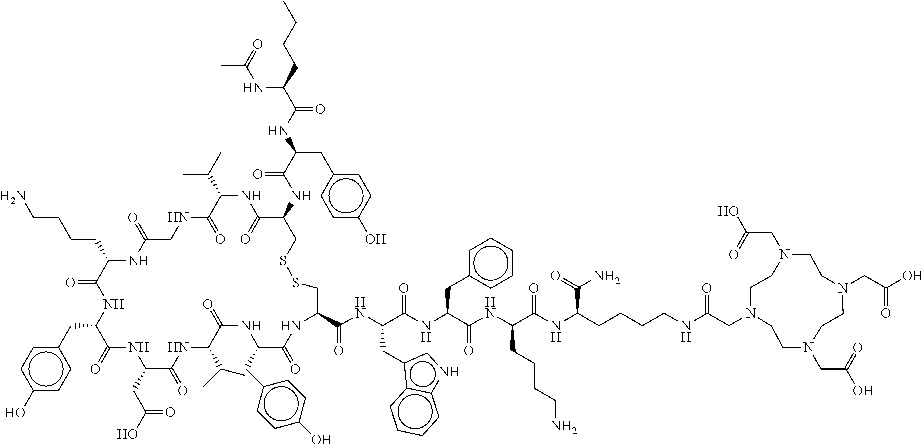 |
| 91 | 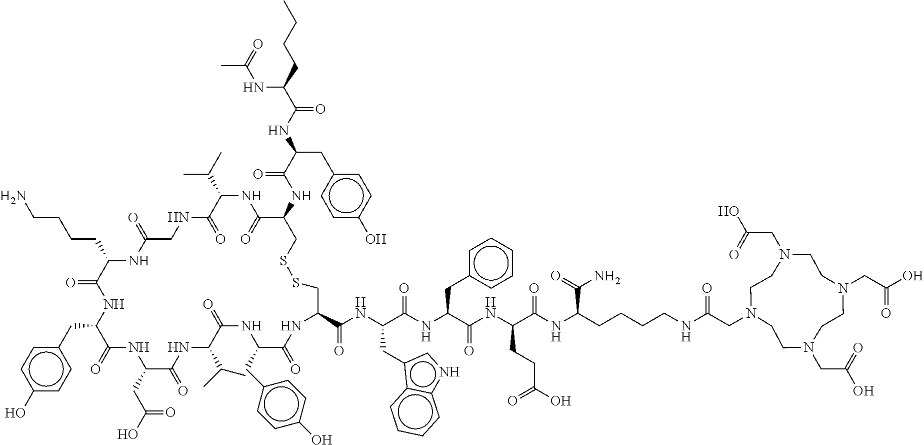 |
| 92 | 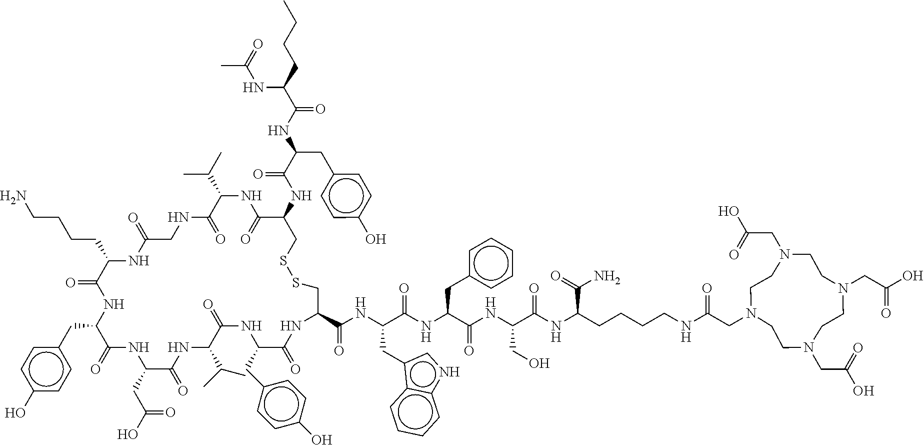 |
| 93 | 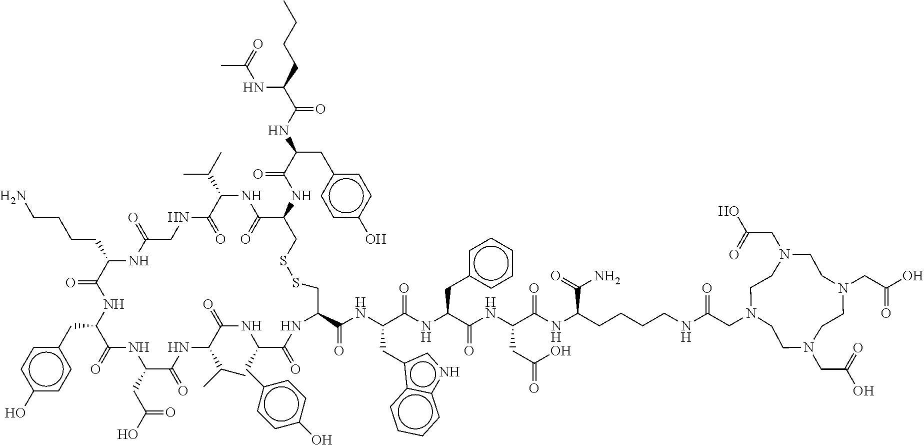 |
| 94 | 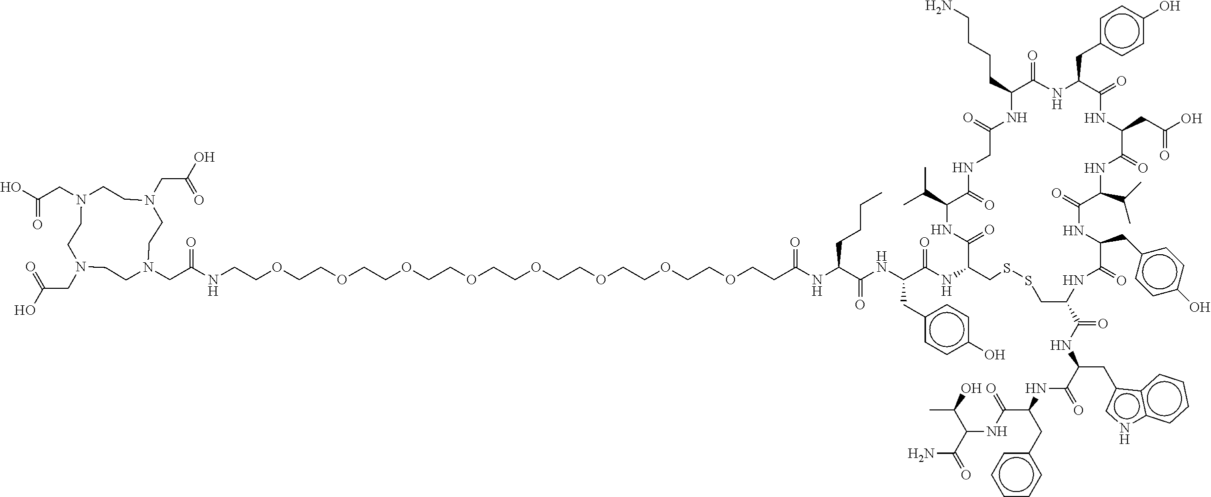 |
| 95 | 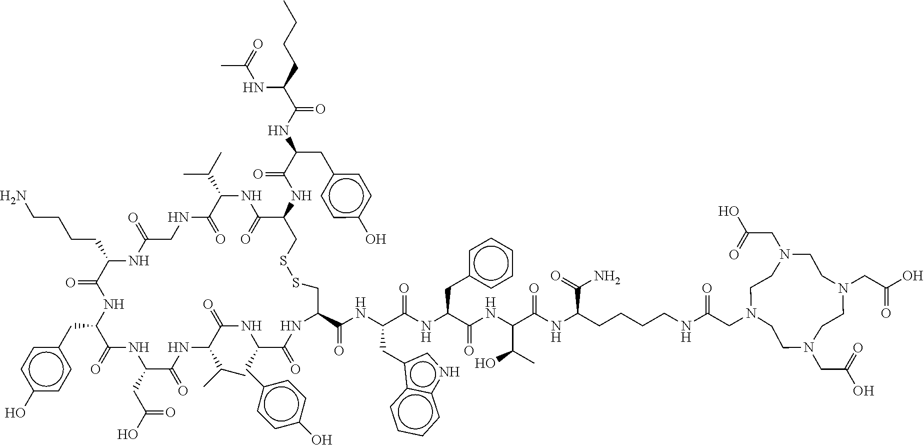 |
| 96 | 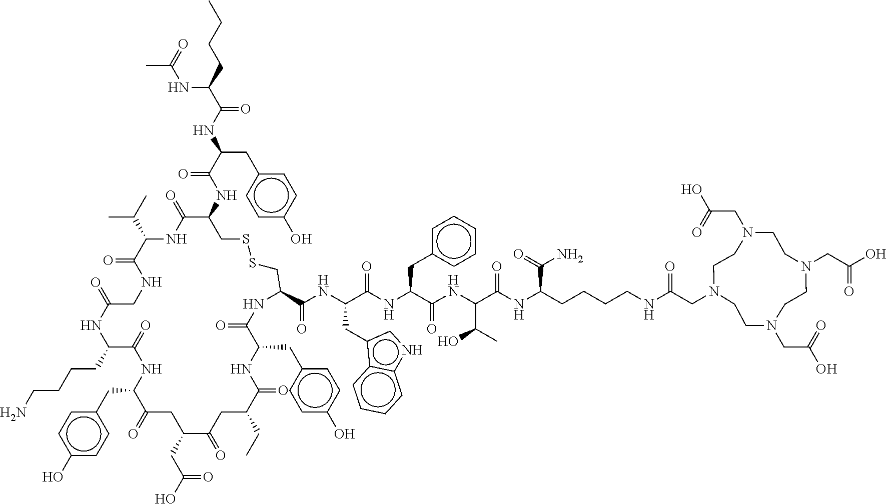 |
| 97 | 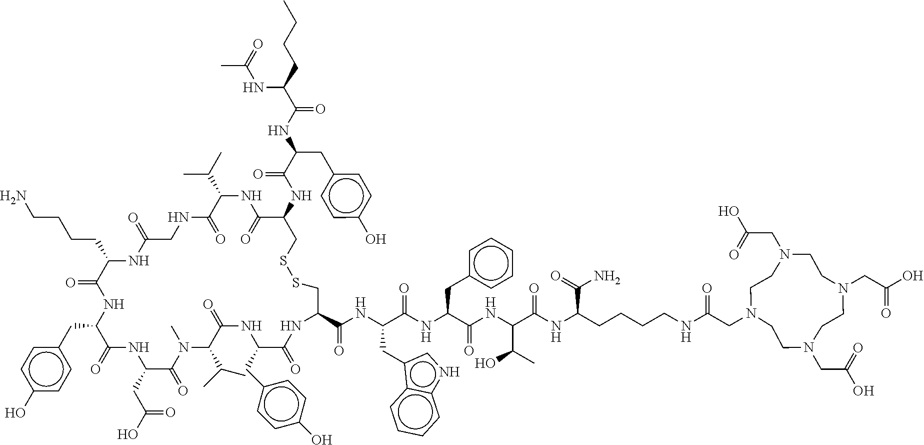 |
| 98 | 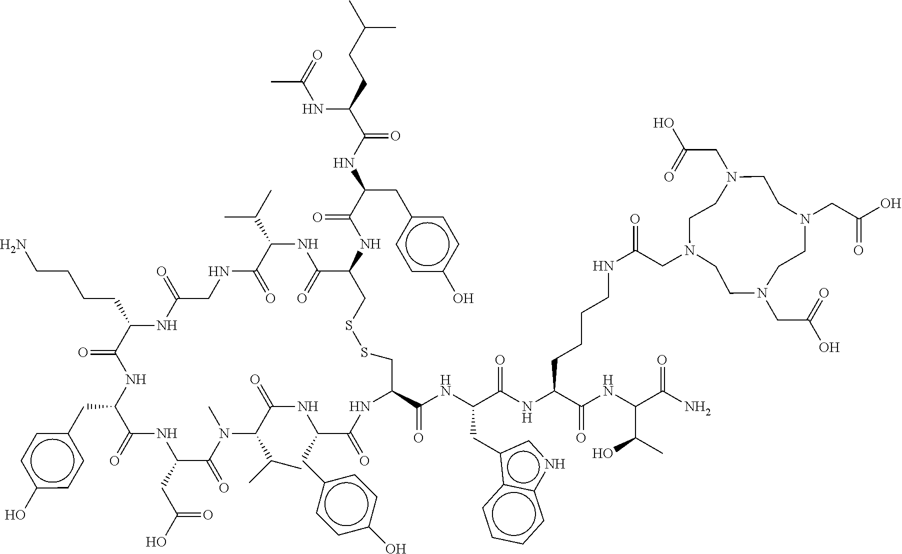 |
| 99 | 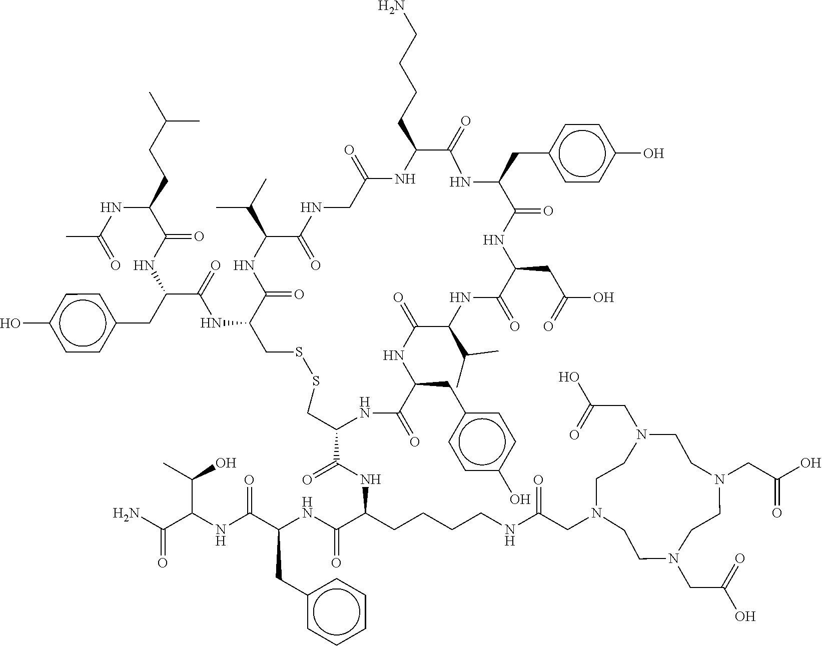 |
| 100 | 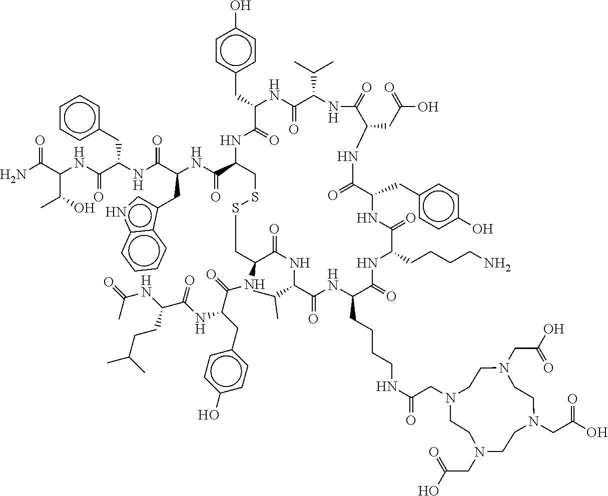 |
| 101 | 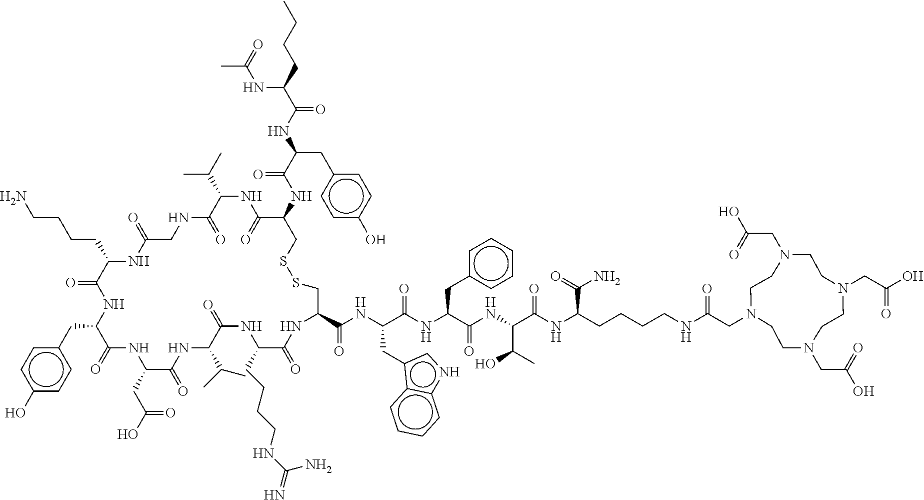 |
| 102 | 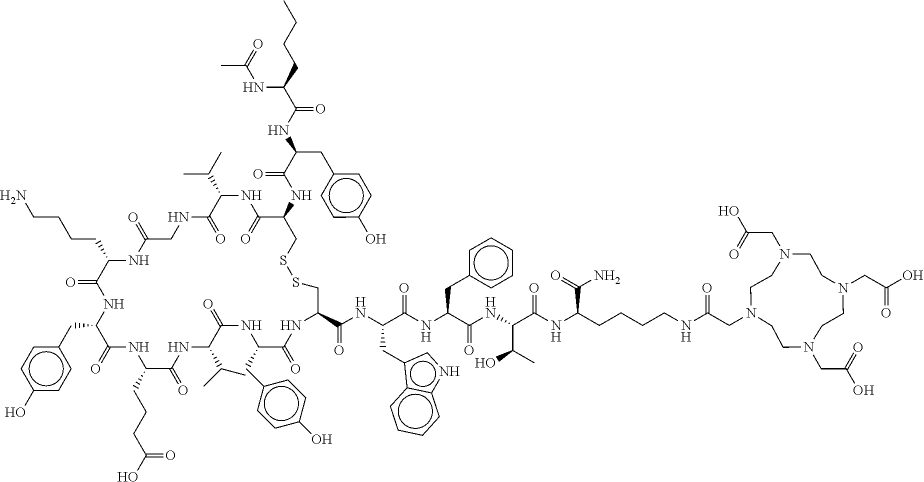 |
| 103 | 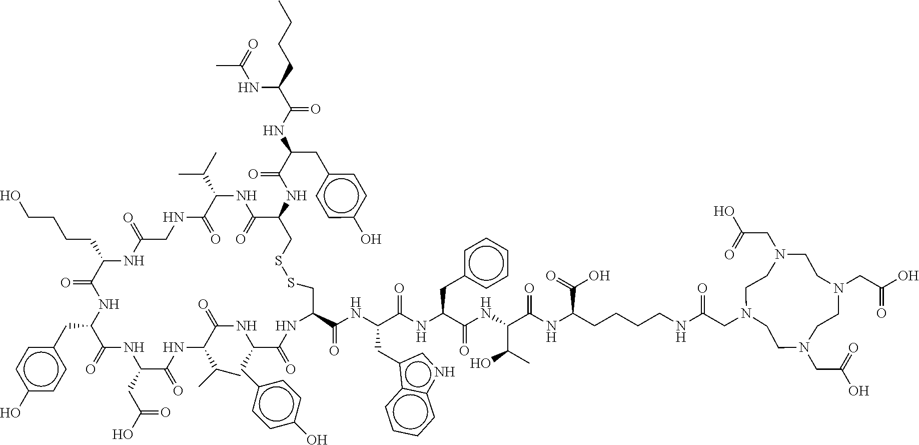 |
| 104 | 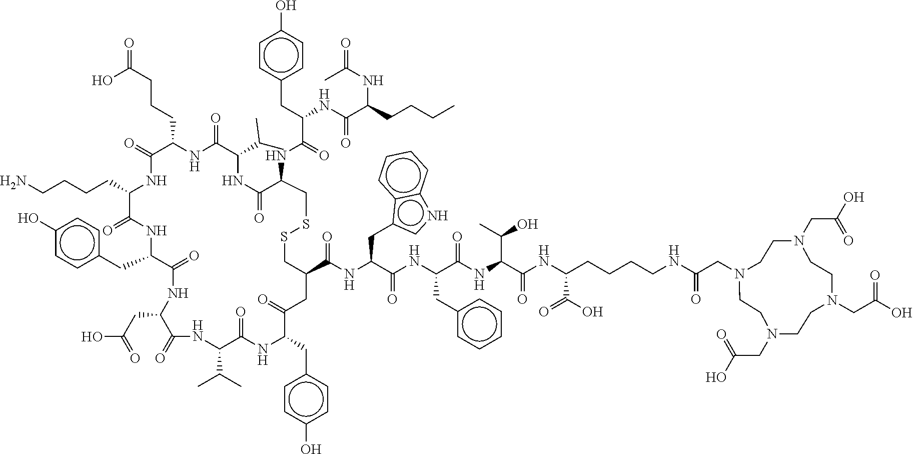 |
| 105 | 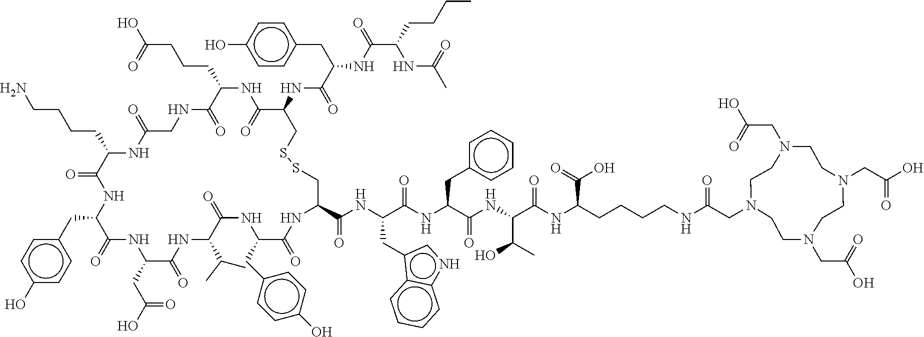 |
| 106 | 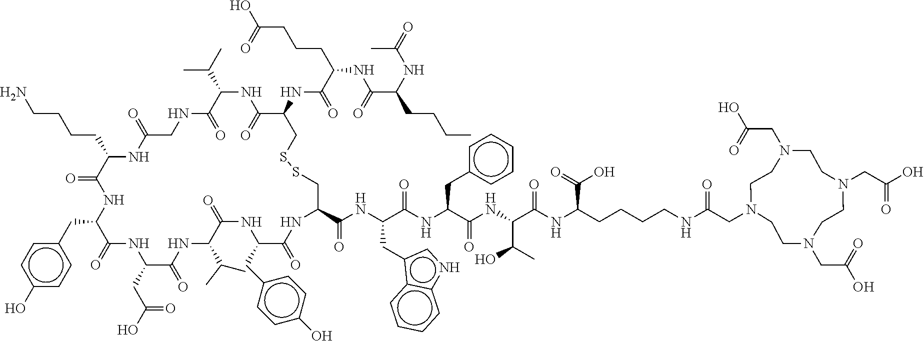 |
| 107 | 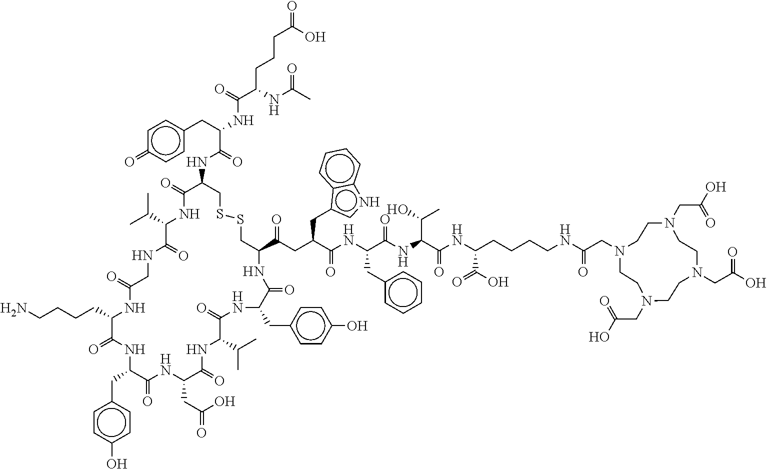 |
| 108 | 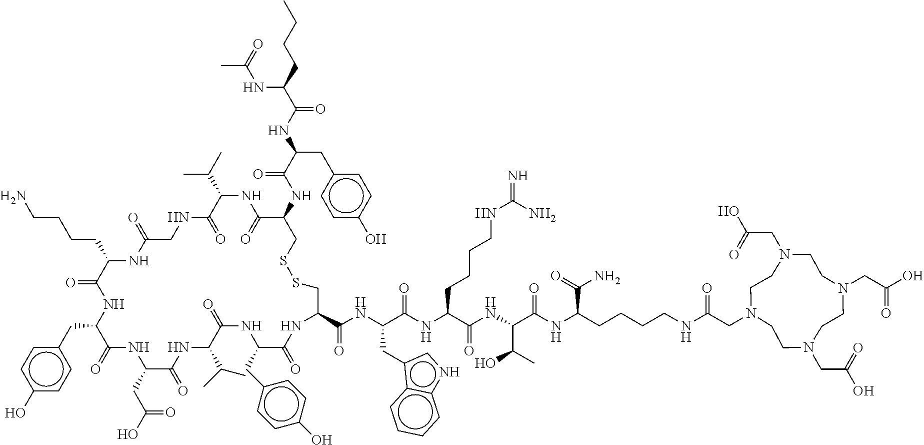 |
| 109 | 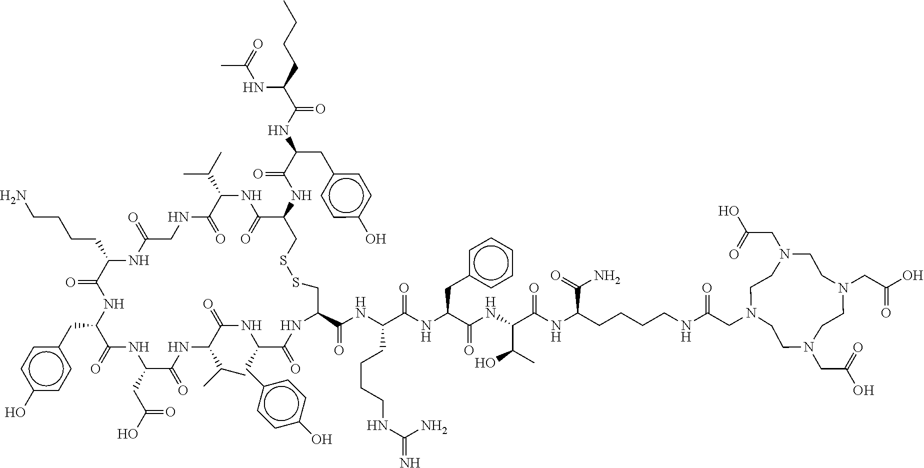 |
| 110 | 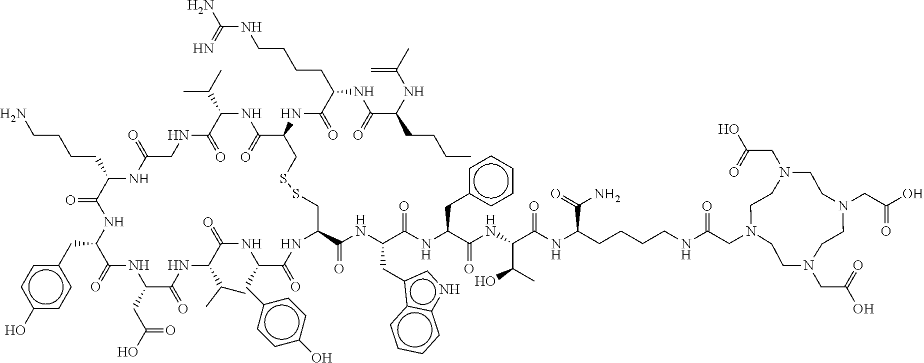 |
| 111 | 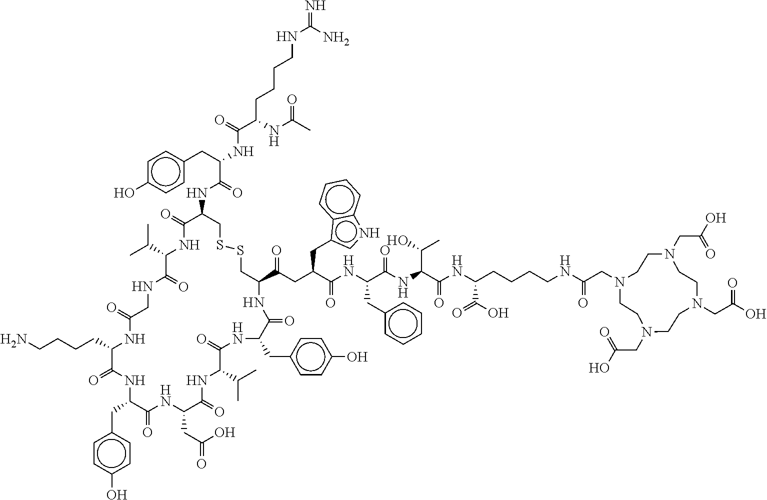 |
| 112 | 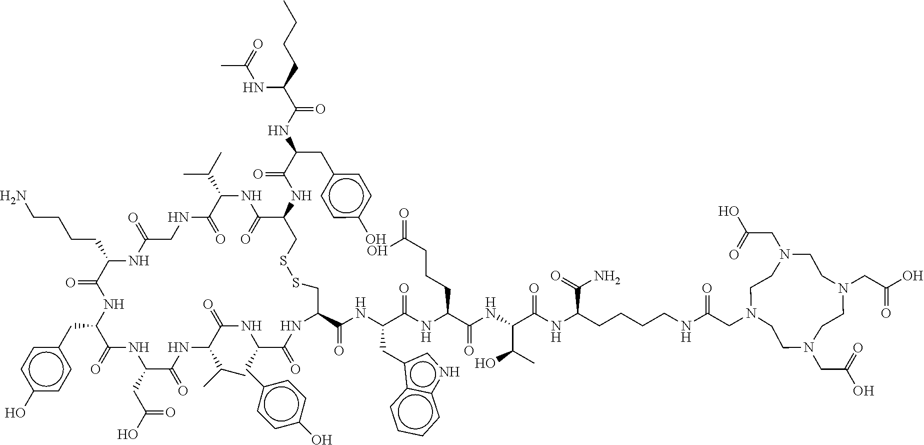 |
| 113 | 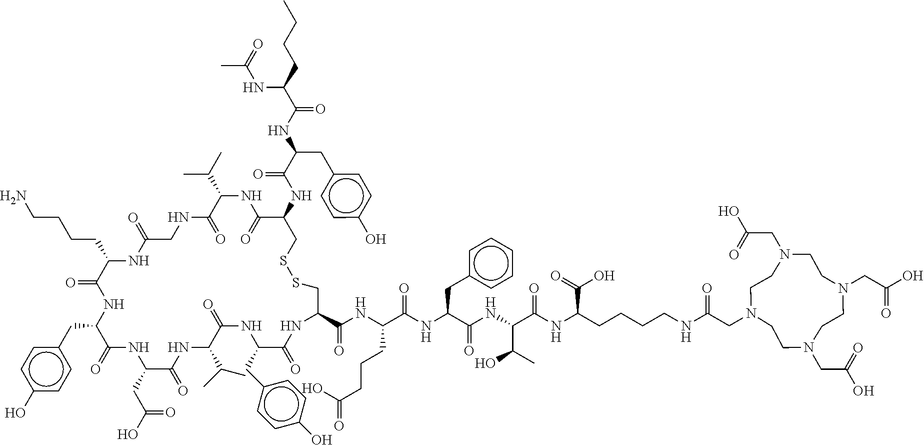 |
| 114 | 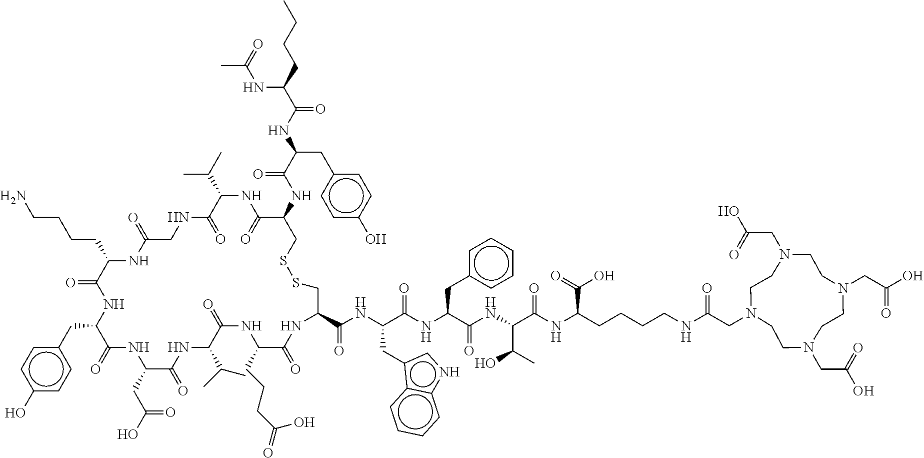 |
| 115 | 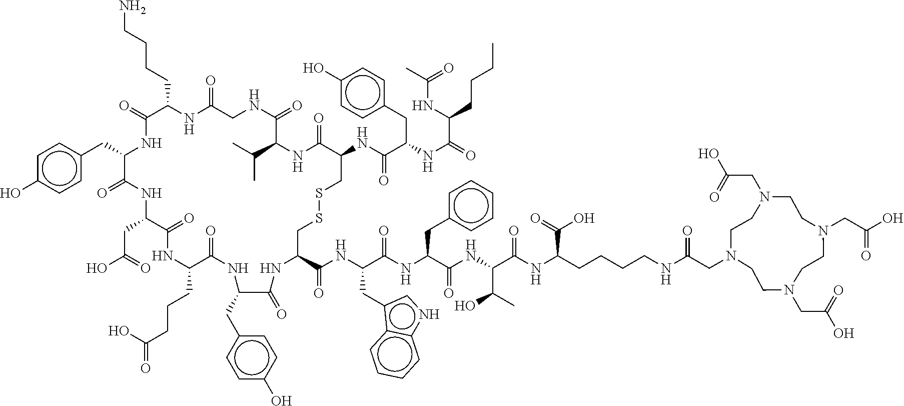 |
| 116 | 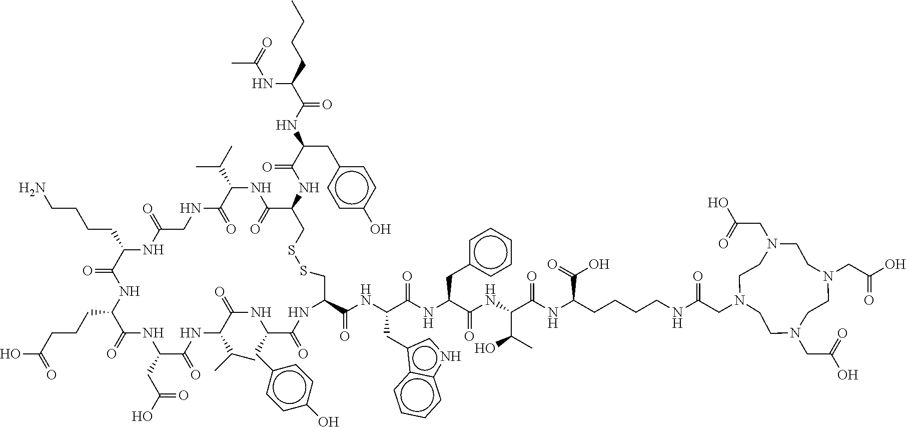 |
| 117 | 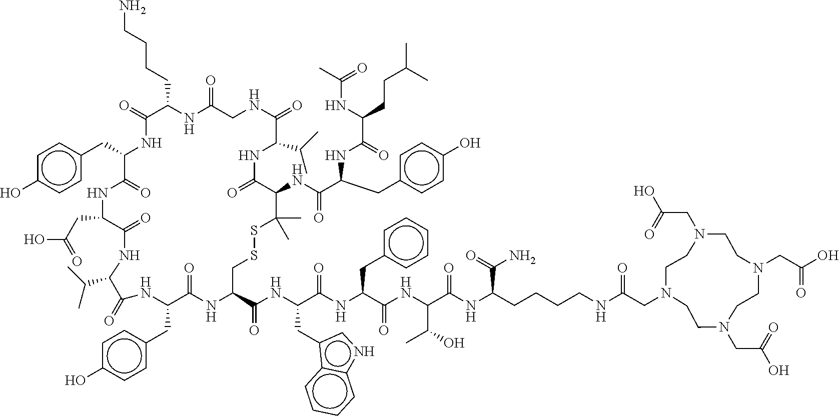 |
| 118 | 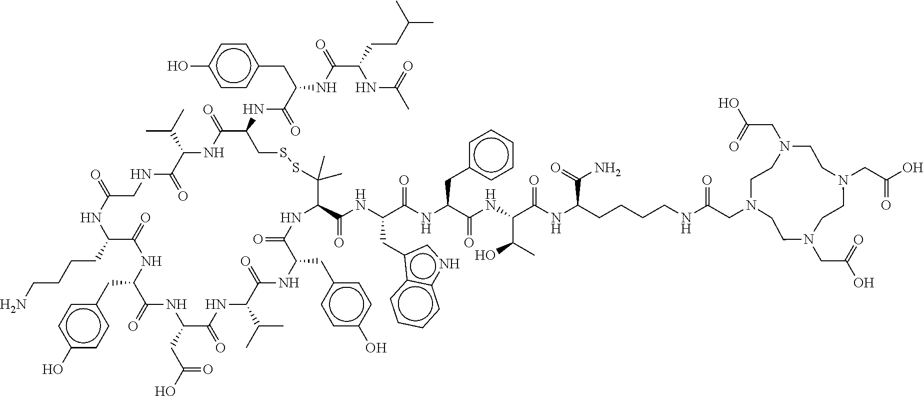 |
| 119 | 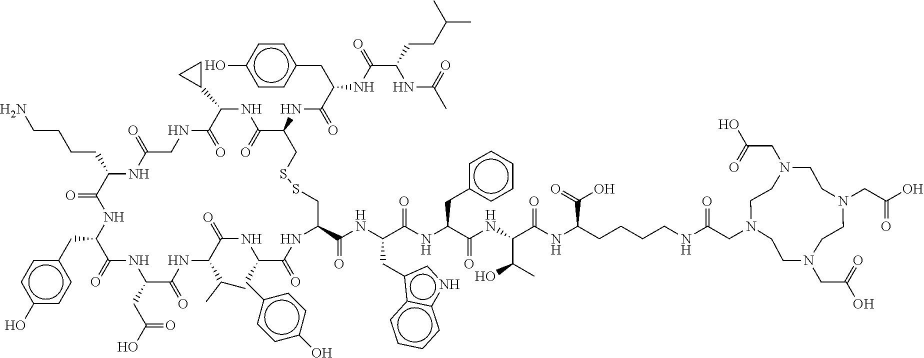 |
| 120 | 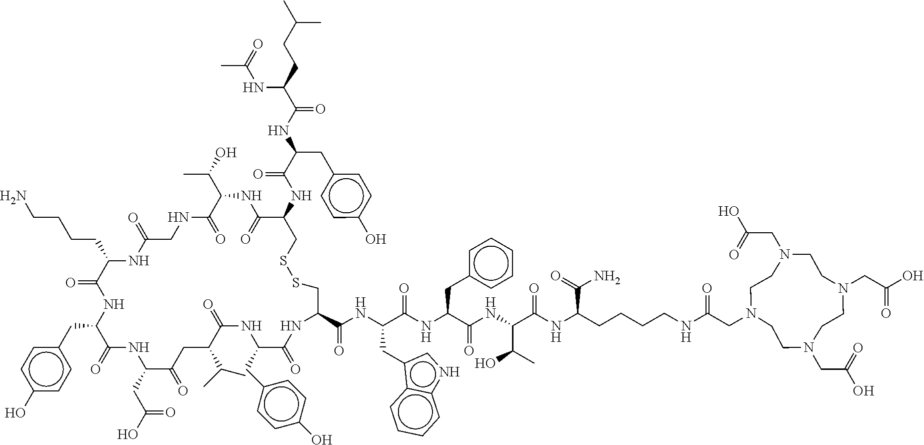 |
| 121 | 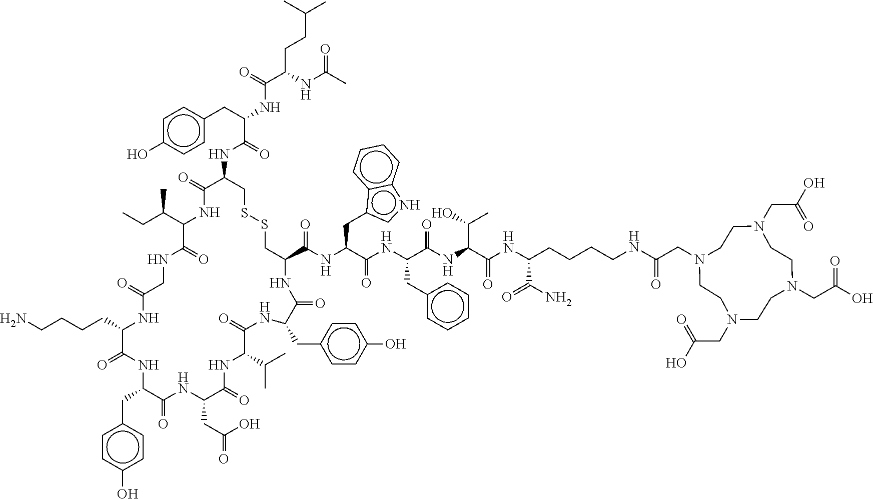 |
| 122 | 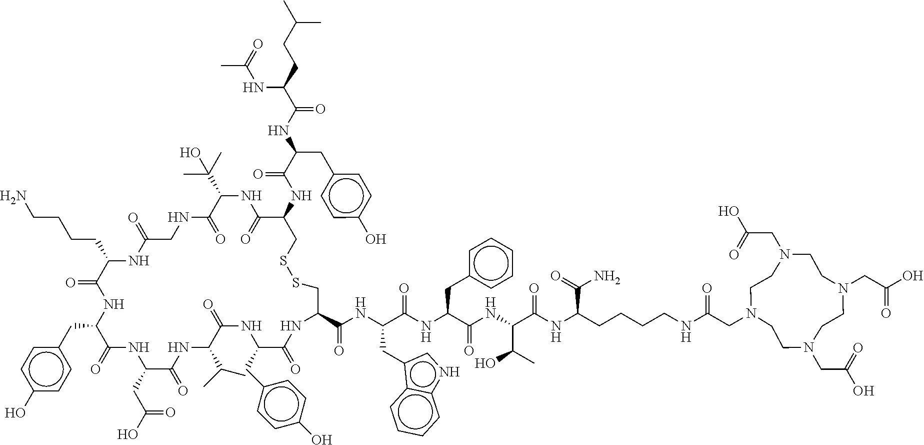 |
| 123 | 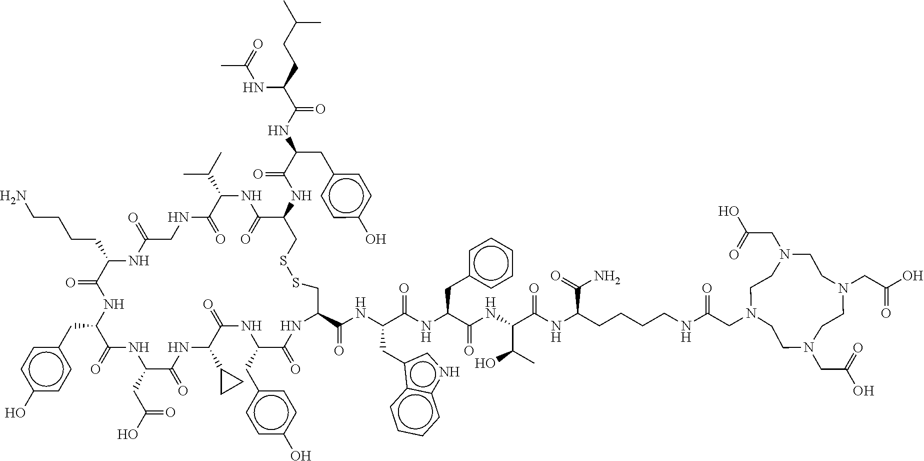 |
| 124 | 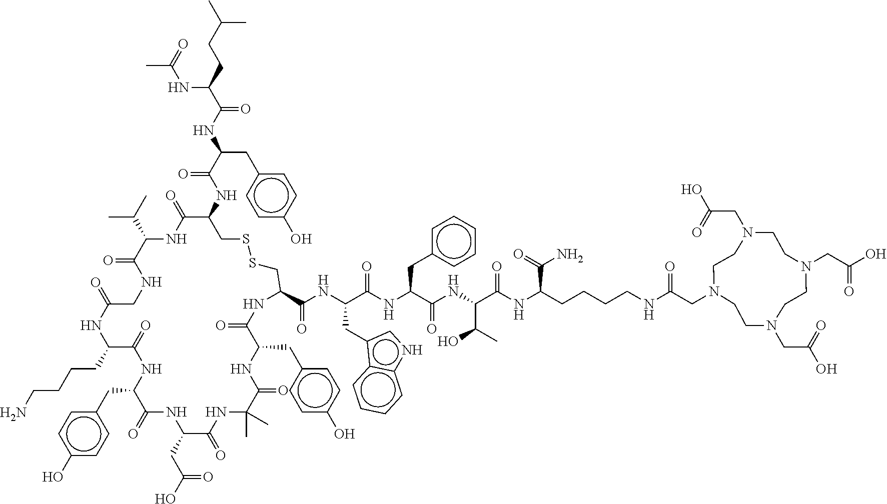 |
| 125 | 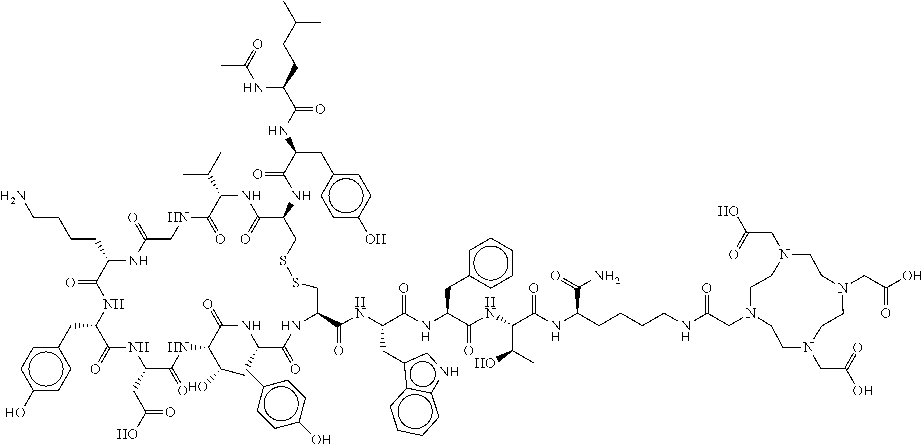 |
| 126 | 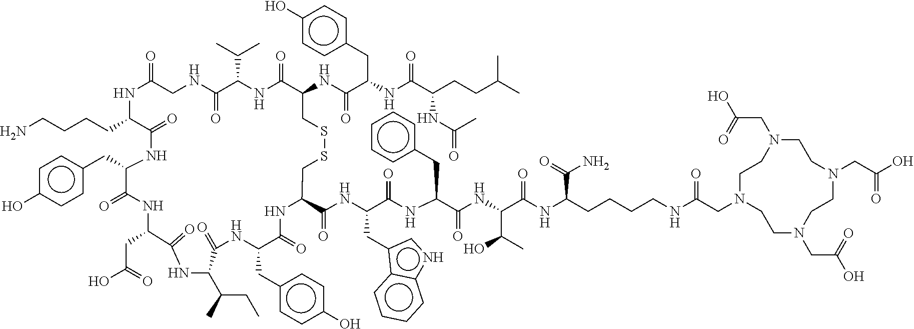 |
| 127 | 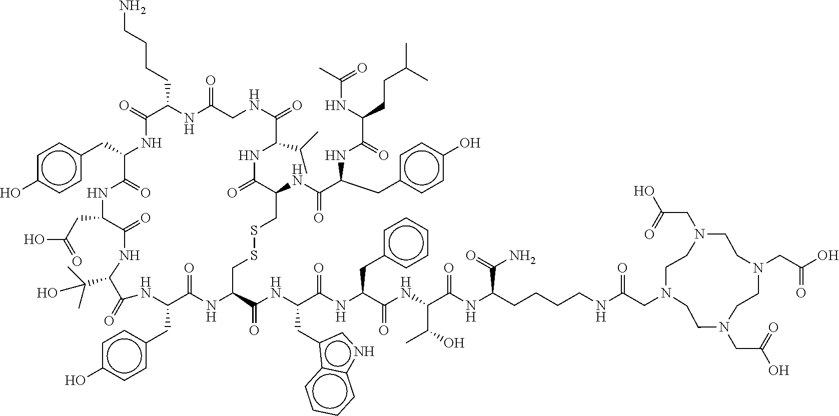 |
| 128 | 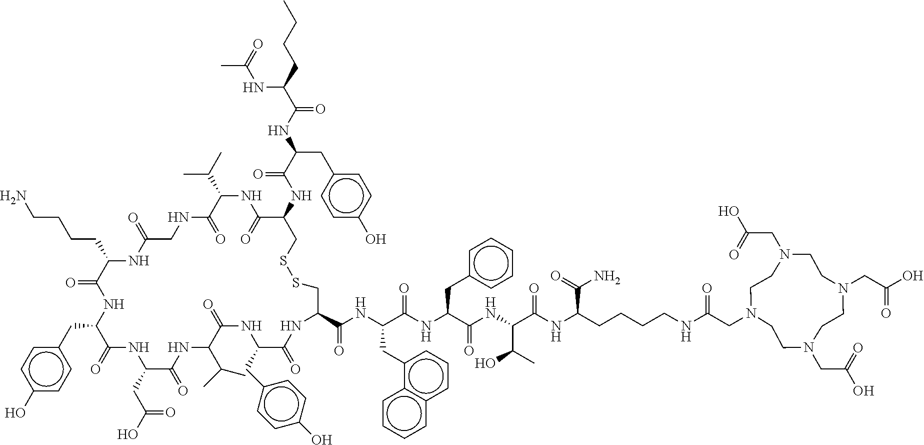 |
| 129 | 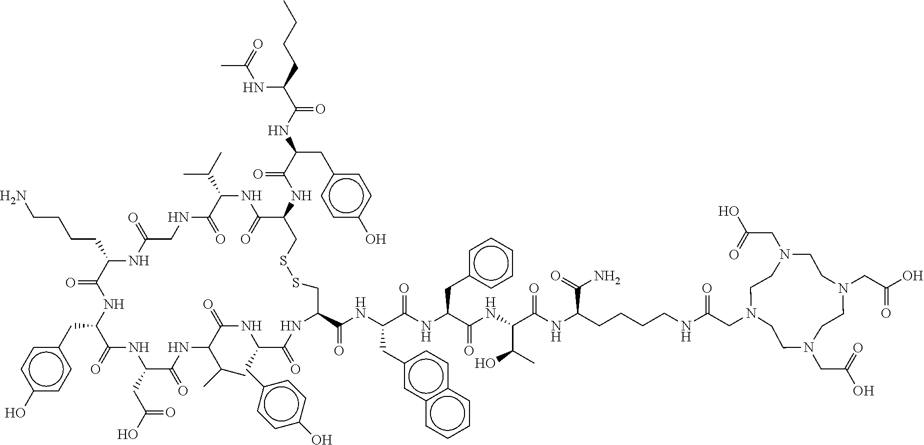 |
| 130 | 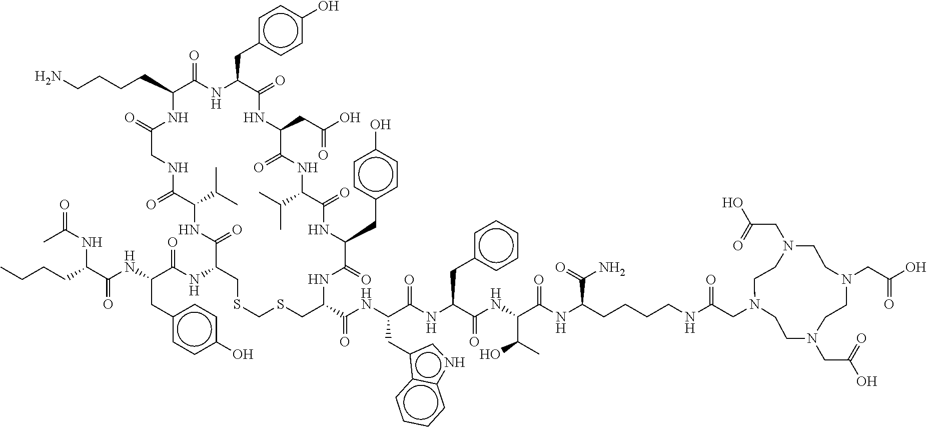 |
| 131 | 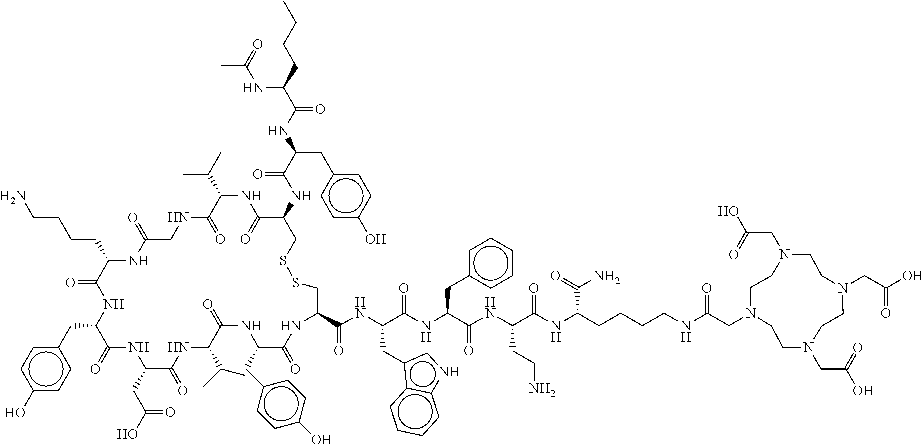 |
| 132 | 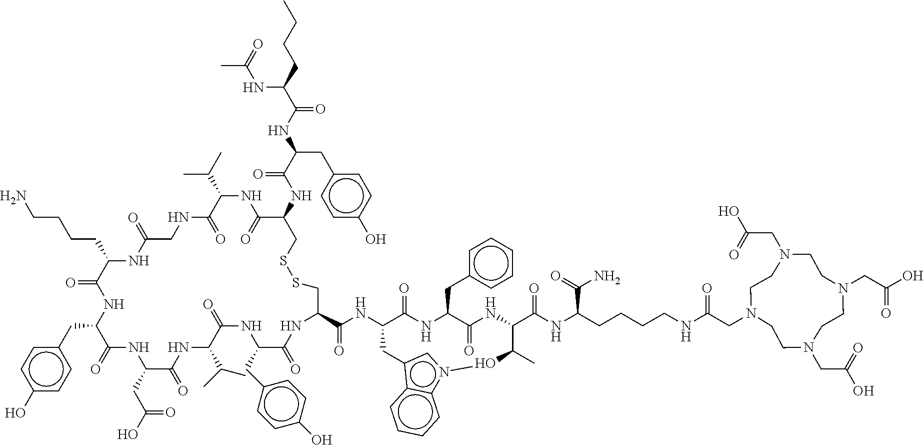 |
| 133 | 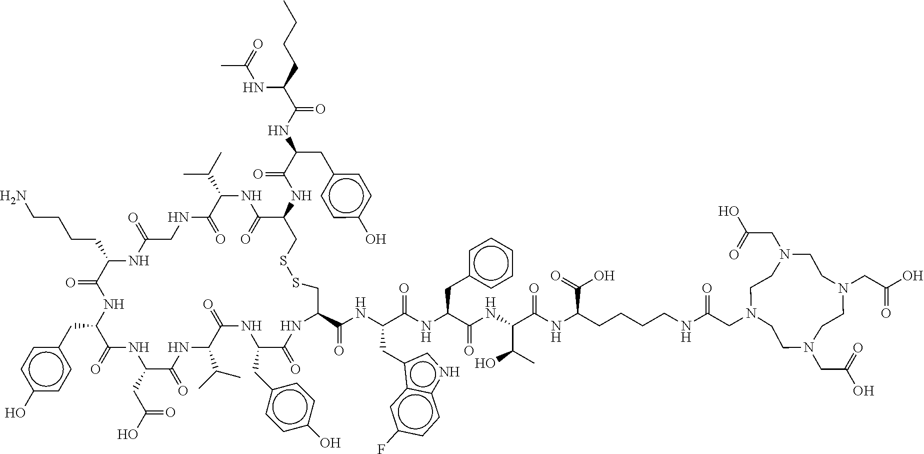 |
| 134 | 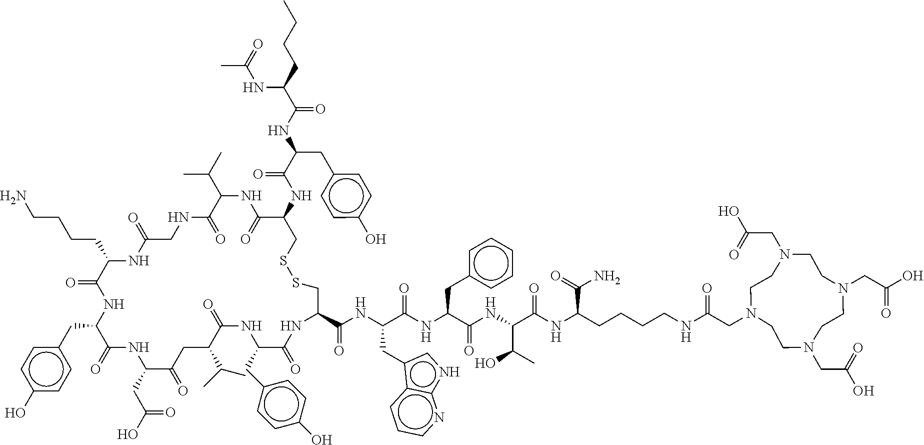 |
| 135 | 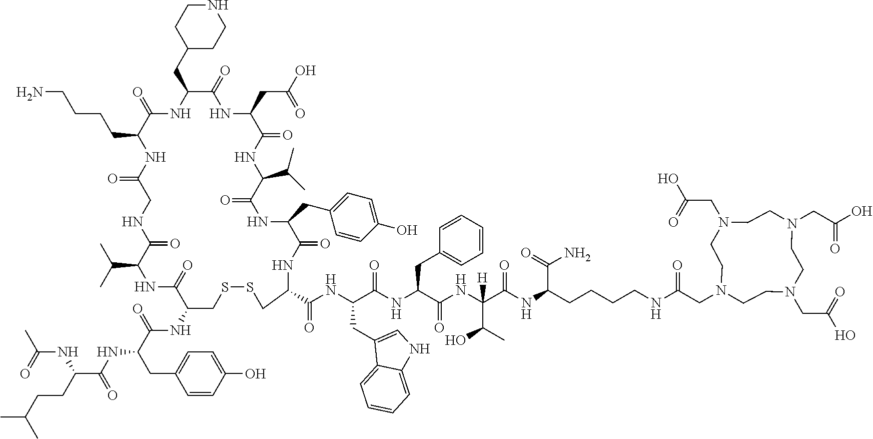 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccccc23)C(=O)N[C@@H](Cc2ccccc2)C(=O)N[C@H](C(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](Cc2ccc(O)cc2)NC(=O)[C@H](C(C)C)NC(=O)[C@H](CC(=O)O)NC(=O)[C@H](CC2CCNCC2)NC(=O)[C@H](CCCCN)NC(=O)CNC(=O)[C@H](C(C)C)NC1=O] |
| 136 | 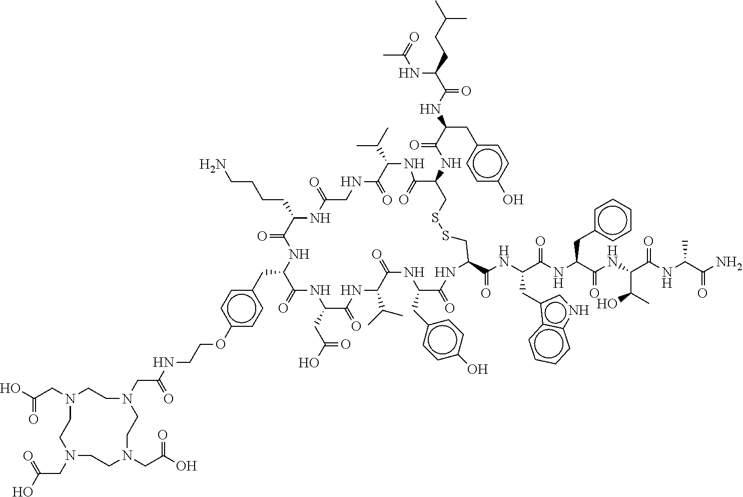 |
| 137 | 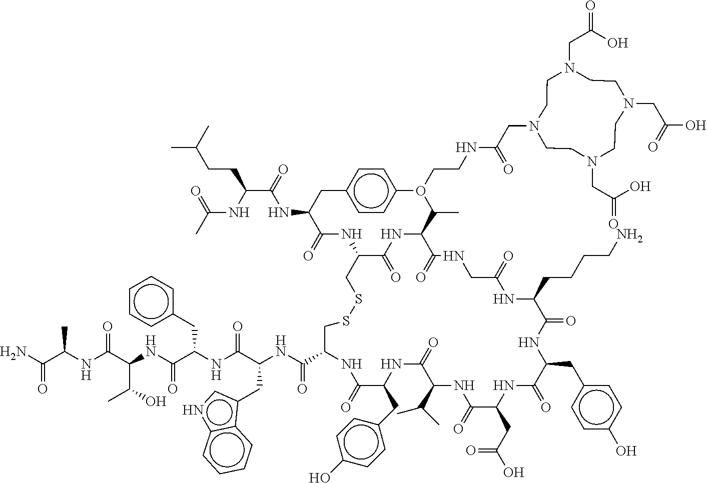 |
| 138 | 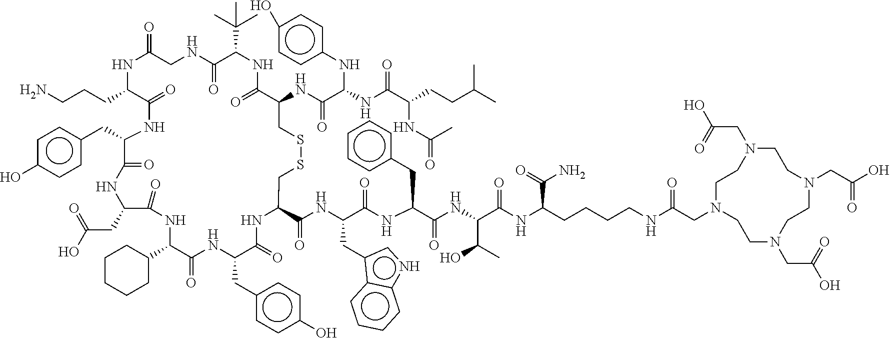 |
| 139 | 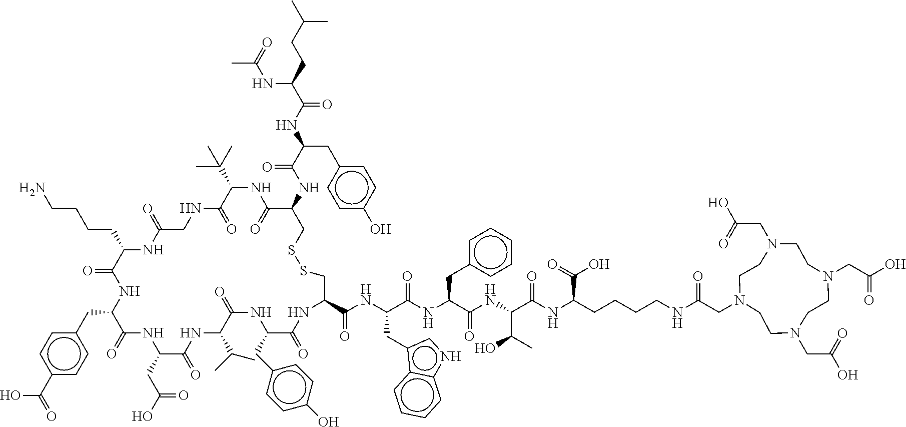 |
| 140 | 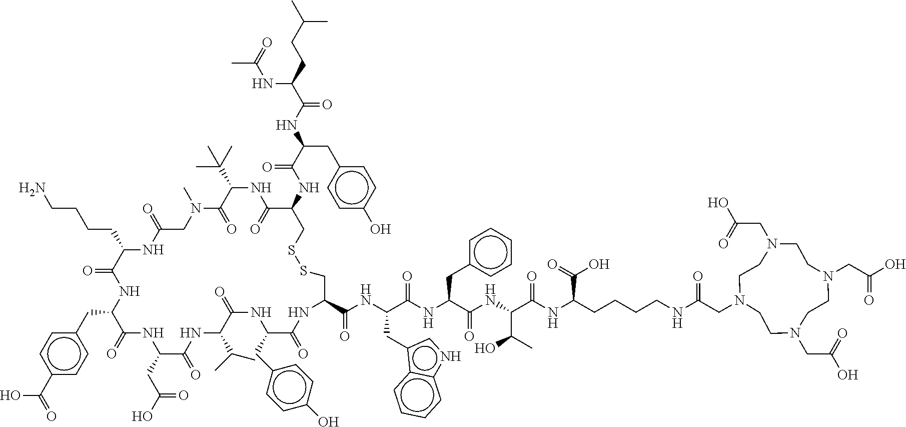 |
| 141 | 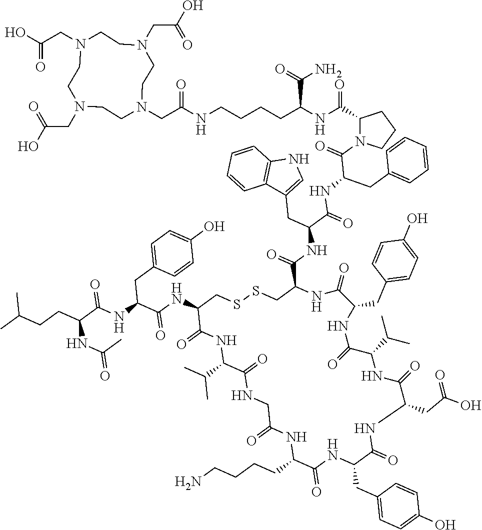 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccccc23)C(=O)N[C@@H](Cc2ccccc2)C(=O)N2CCC[C@H]2C(=O)N[C@@H](CCCCNC(=O)CN2CCN(CC(=O)O)CCN(CC(=O)O)CCN(CC(=O)O)CC2)C(N)=O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCN)NC(=O)CNC(=O)[C@H](C(C)C)NC1=O] |
| 142 | 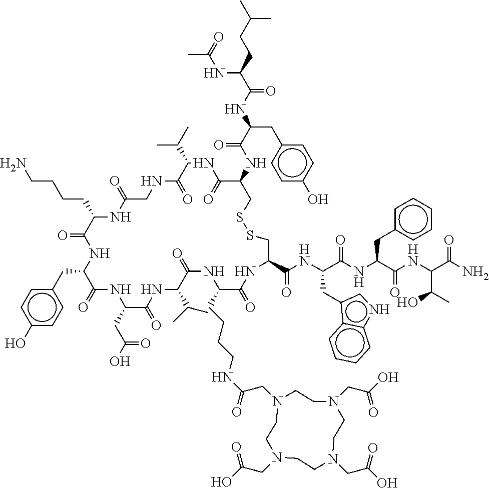 |
| 143 | 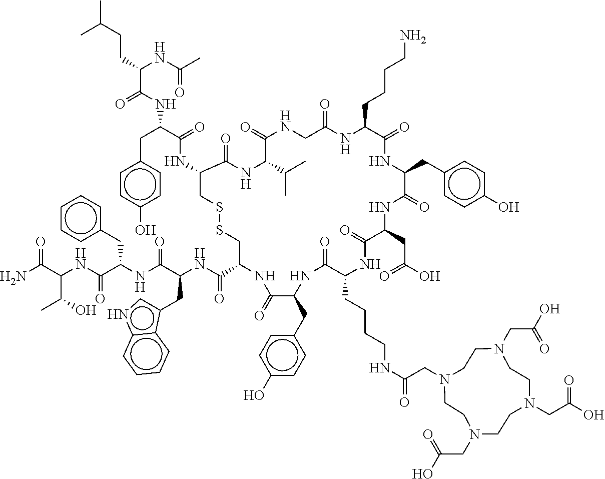 |
| 144 | 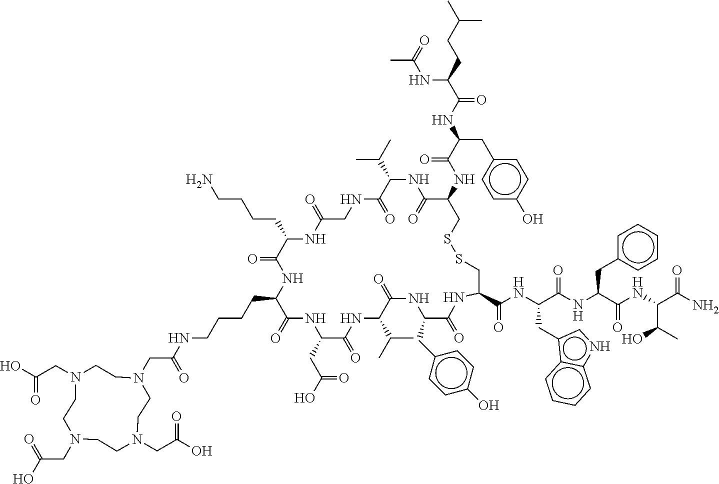 |
| 145 | 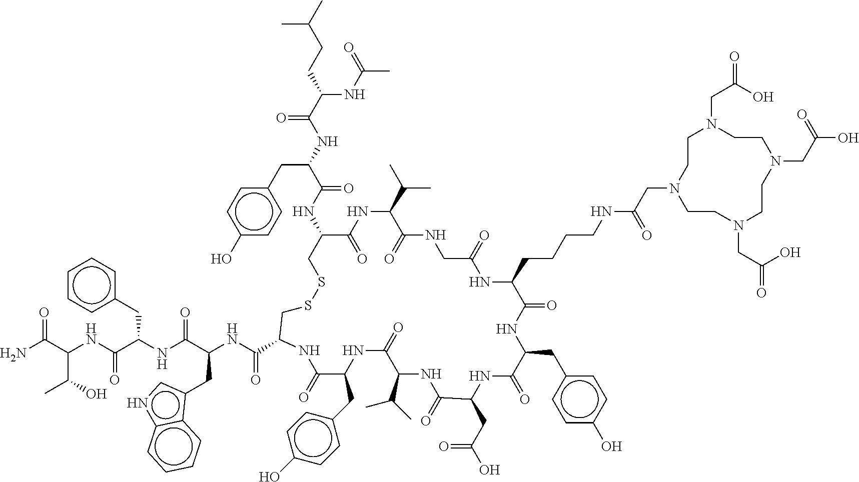 |
| 146 | 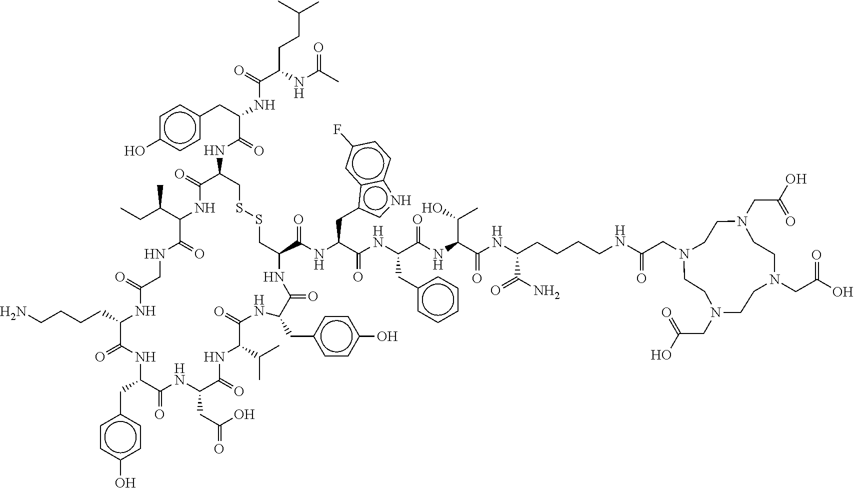 |
| 147 | 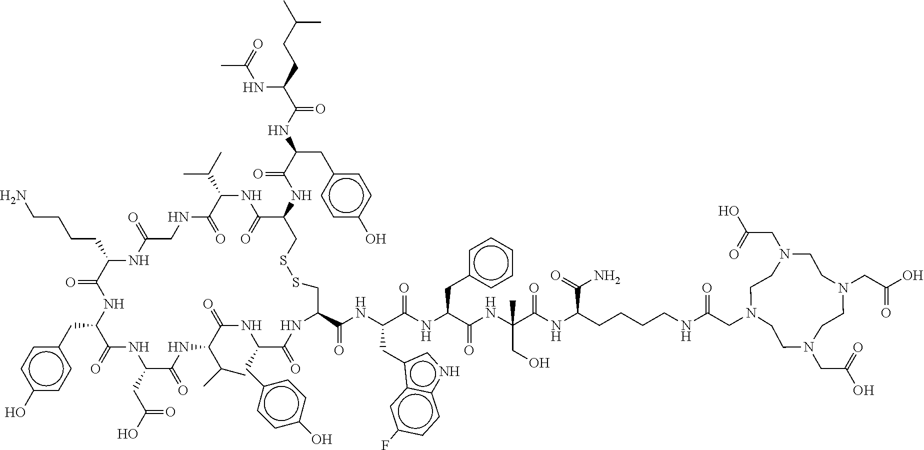 |
| 148 | 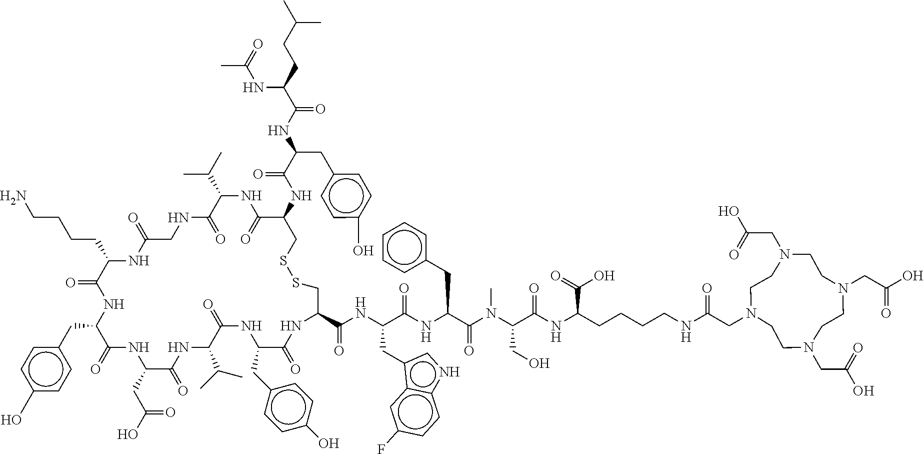 |
| 149 | 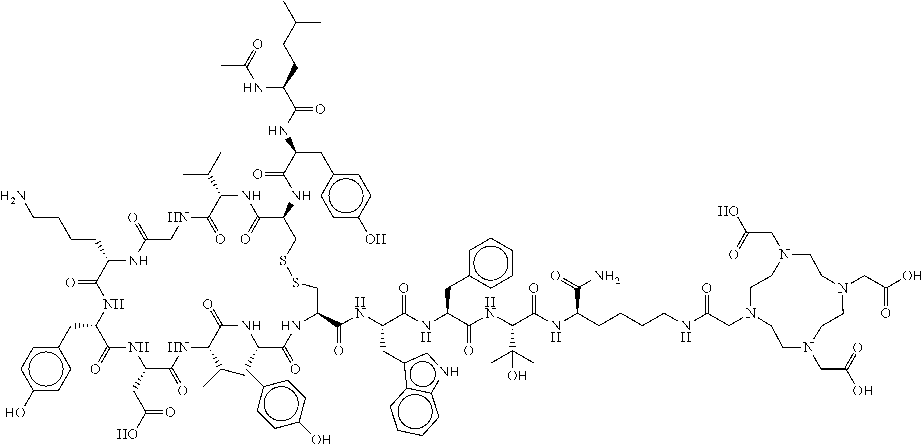 |
| 150 | 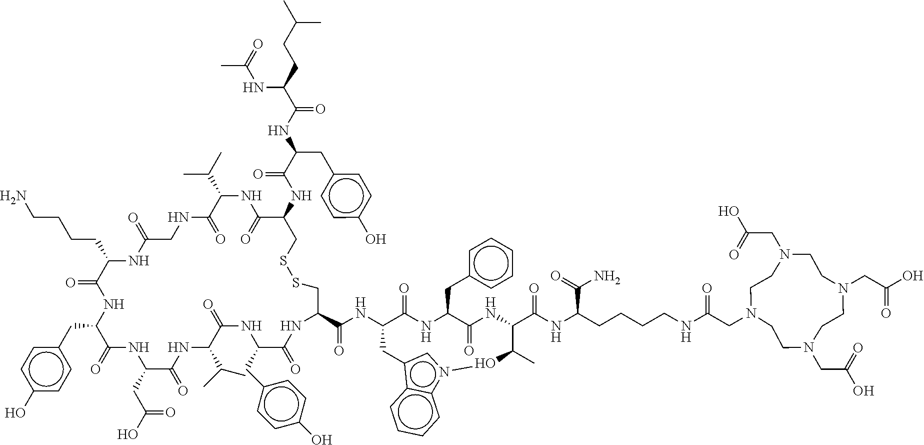 |
| 151 | 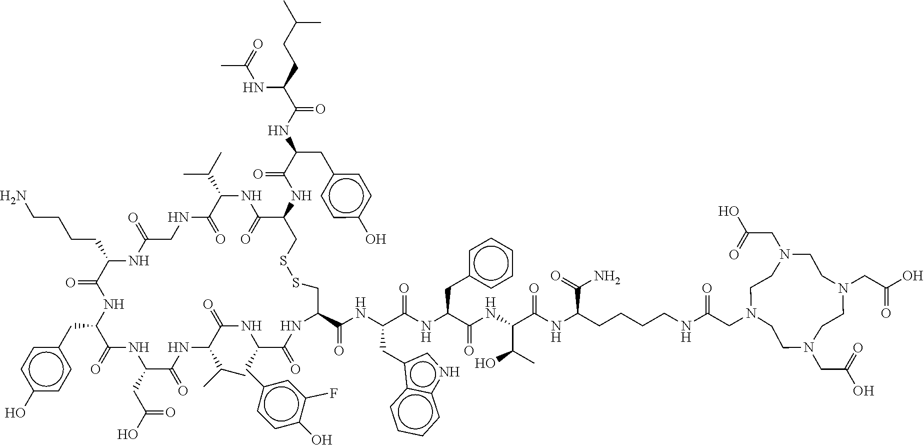 |
| 152 | 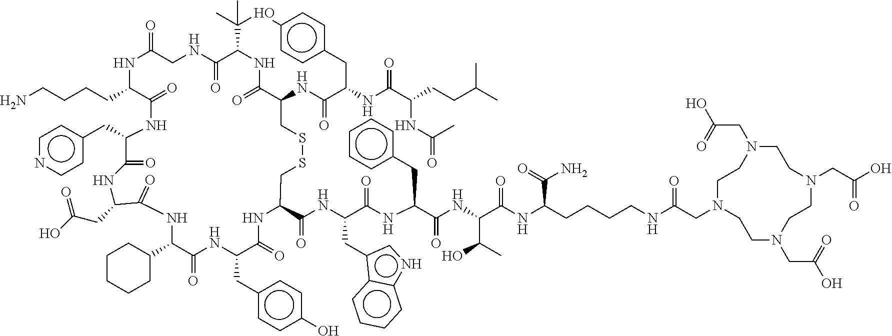 |
| 153 | 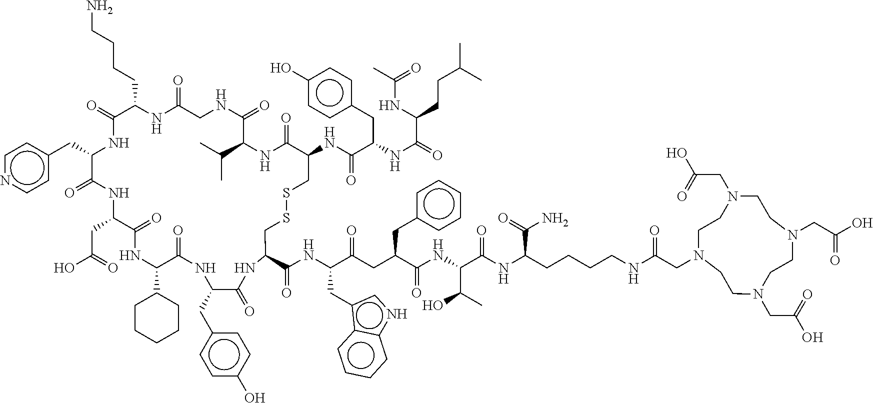 |
| 154 | 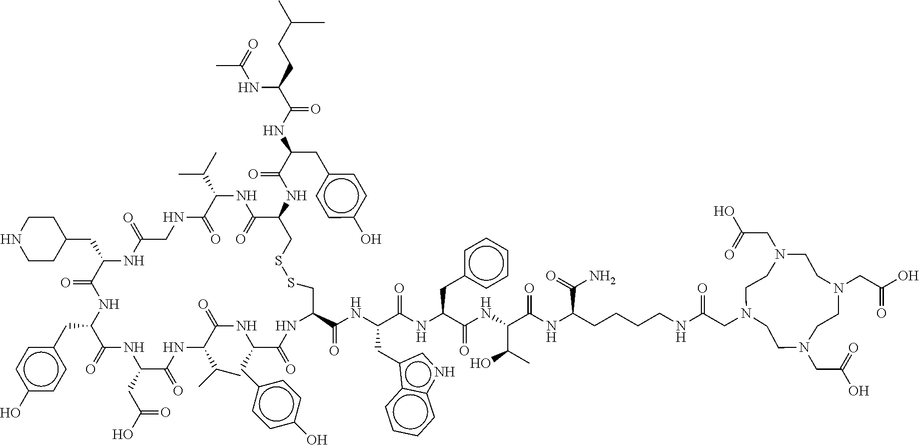 |
| 155 | 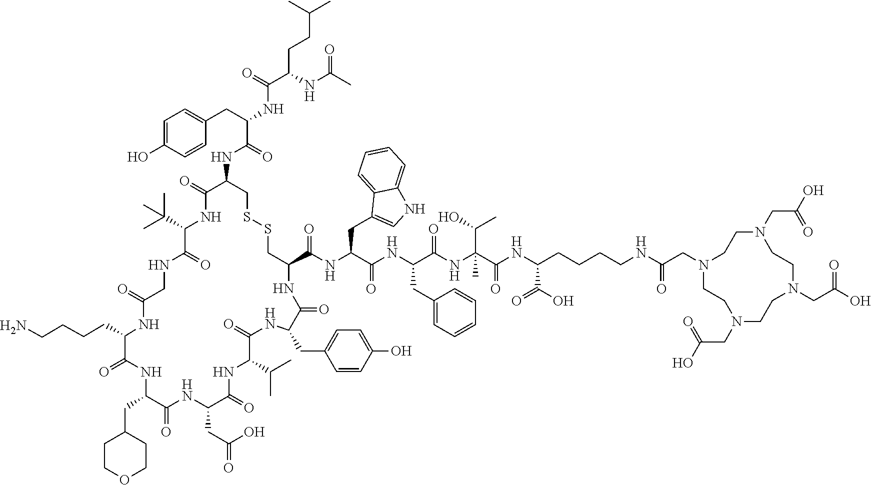 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccccc23)C(=O)N[C@@H](Cc2ccccc2)C(=O)N[C@](C)(C(=O)N[C@H](CCCCNC(=O)CN2CCN(CC(=O)O)CCN(CC(=O)O)CCN(CC(=O)O)CC2)C(=O)O)[C@@H](C)O)NC(=O)[C@H](Cc2ccc(O)cc2)NC(=O)[C@H](C(C)C)NC(=O)[C@H](CC(=O)O)NC(=O)[C@H](CC2CCOCC2)NC(=O)[C@H](CCCCN)NC(=O)CNC(=O)[C@H](C(C)(C)C)NC1=O] |
| 156 | 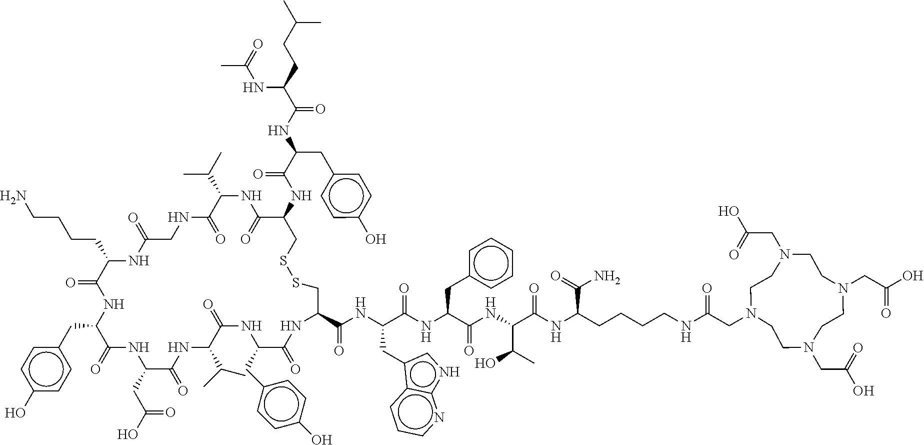 |
| 157 | 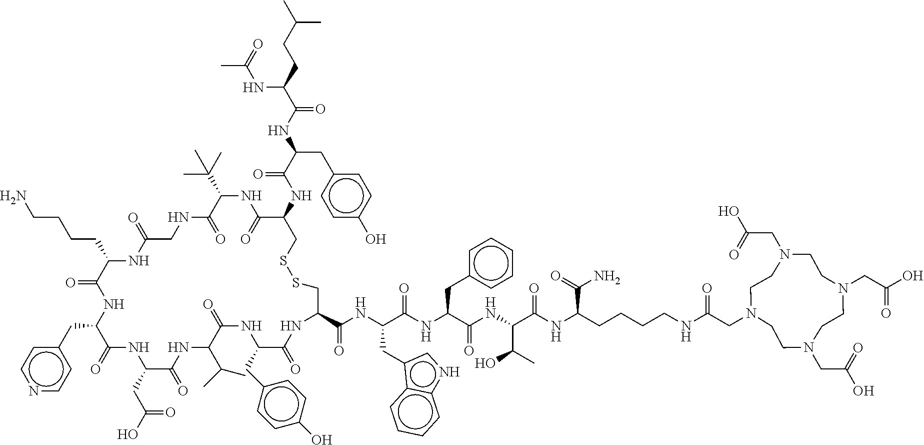 |
| 158 | 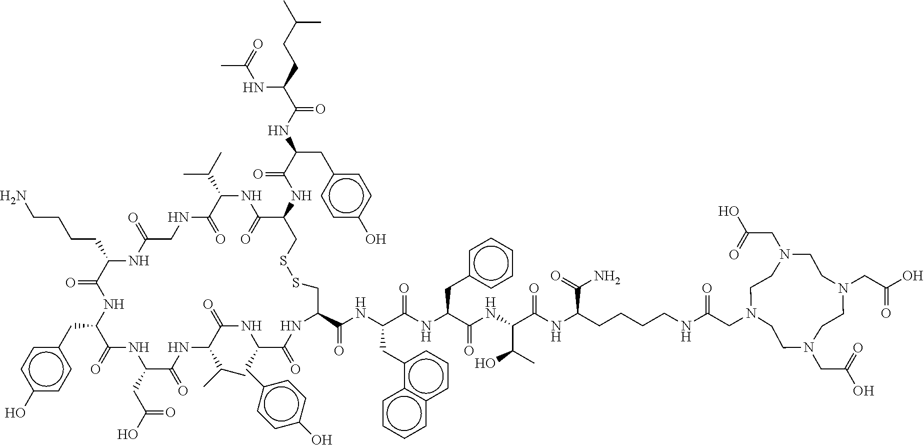 |
| 159 | 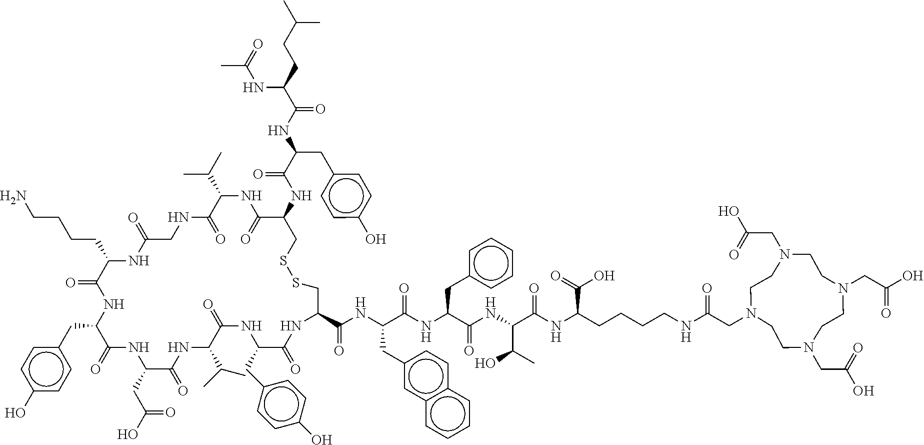 |
| 160 | 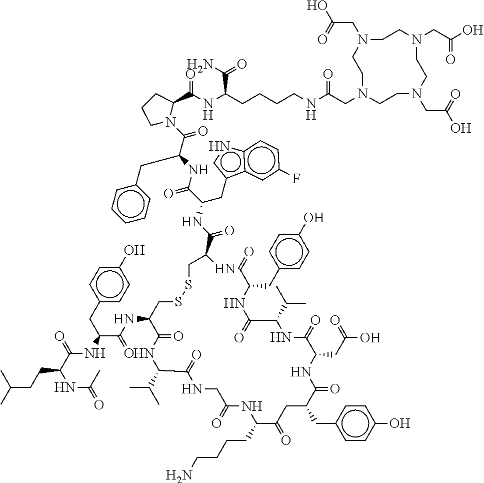 |
| 161 | 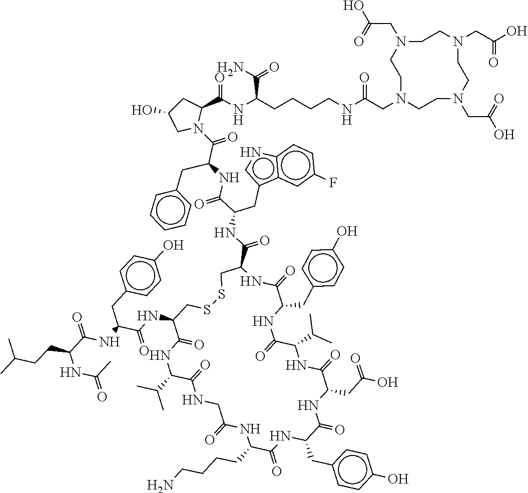 |
| 162 | 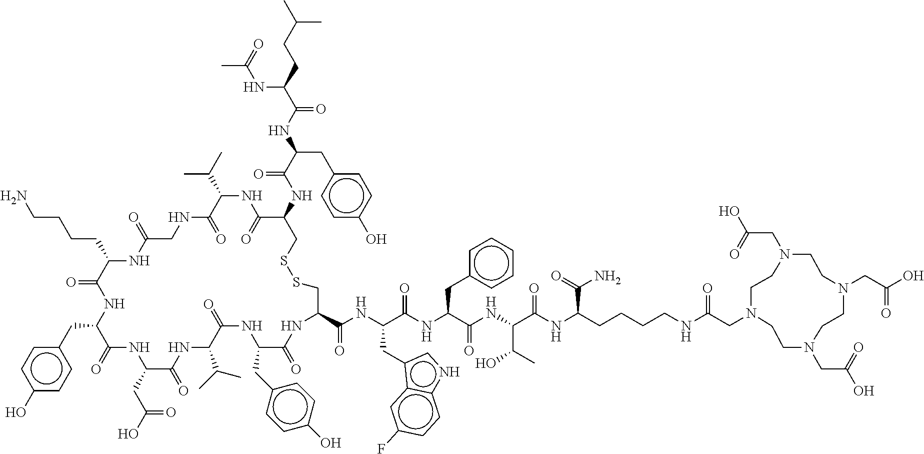 |
| 163 | 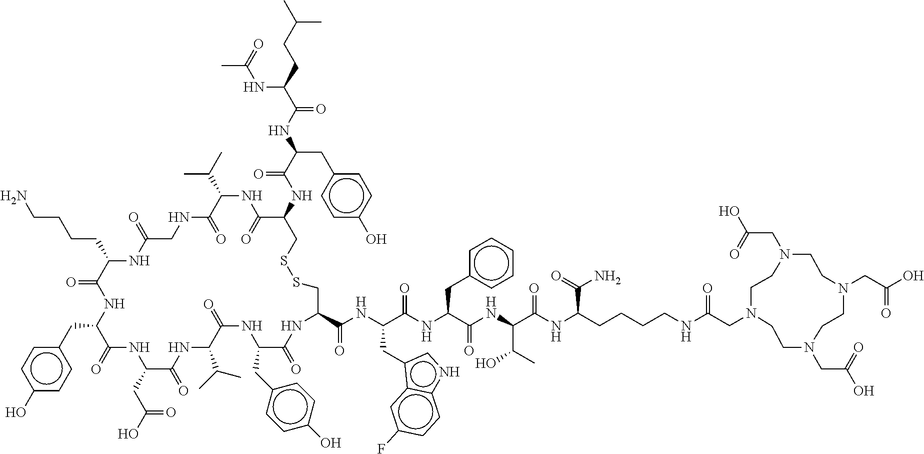 |
| 164 | 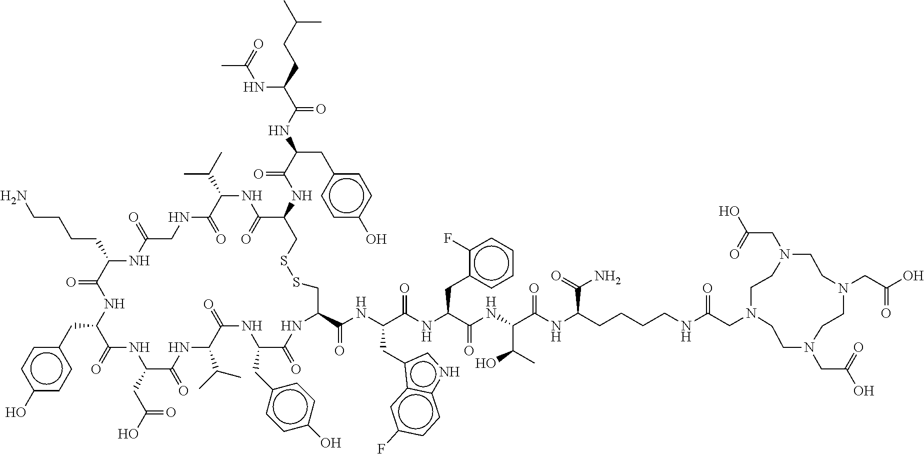 |
| 165 | 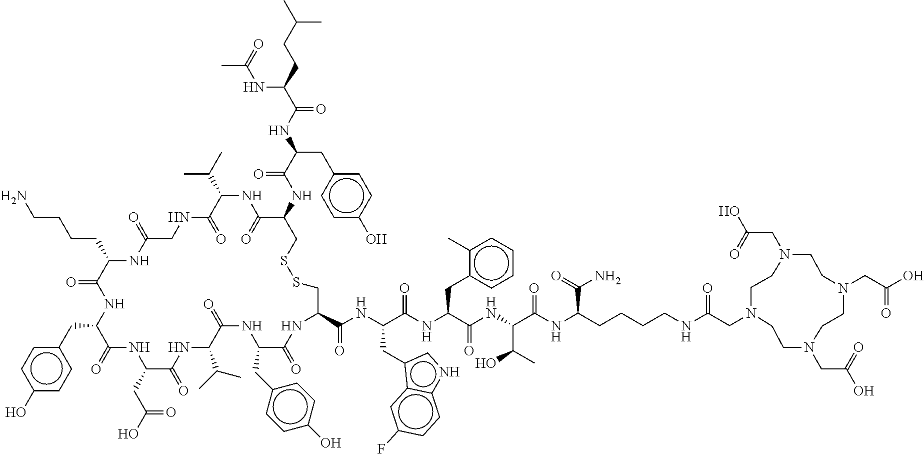 |
| 166 | 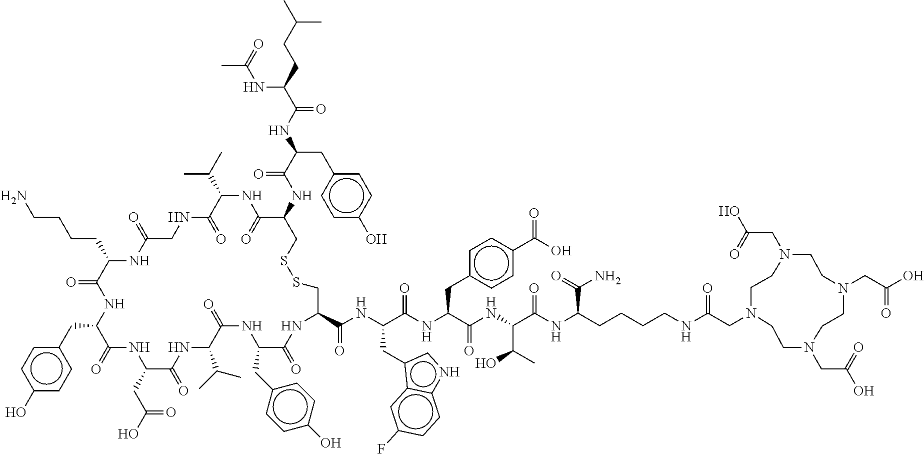 |
| 167 | 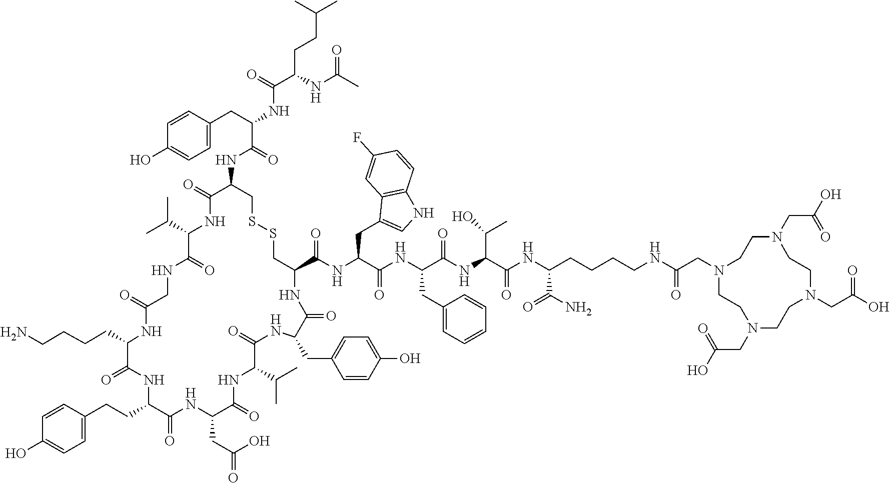 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@@H](Cc2ccccc2)C(=O)N[C@H](C(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](Cc2ccc(O)cc2)NC(=O)[C@H](C(C)C)NC(=O)[C@H](CC(=O)O)NC(=O)[C@H](CCc2ccc(O)cc2)NC(=O)[C@H](CCCCN)NC(=O)CNC(=O)[C@H](C(C)C)NC1=O] |
| 168 | 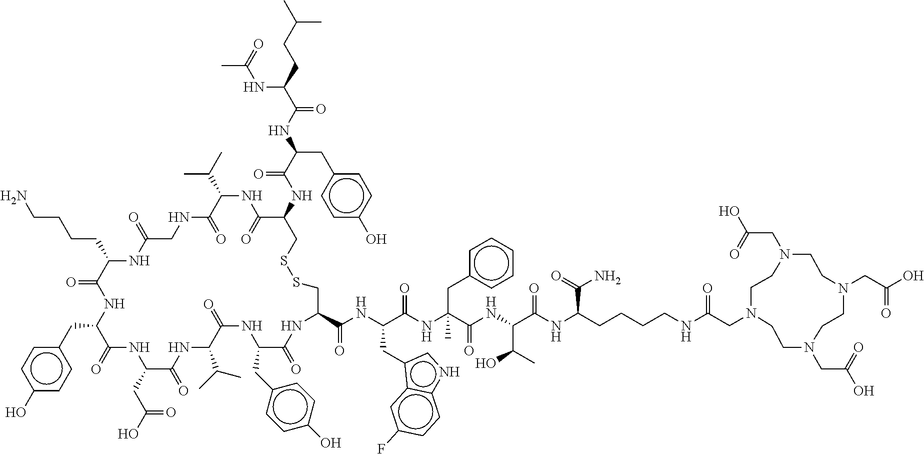 |
| 169 | 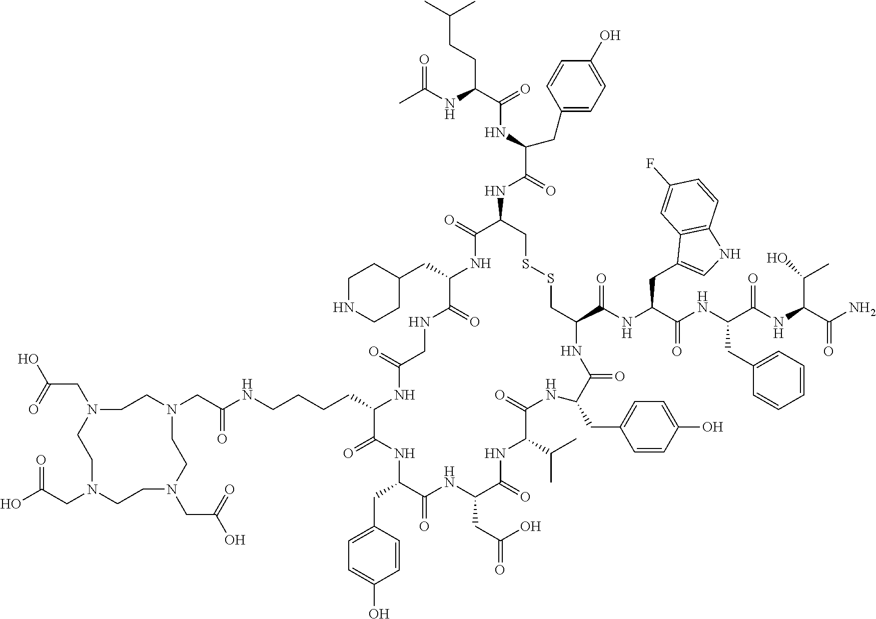 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@@H](Cc2ccccc2)C(=O)N[C@H](C(N)=O)[C@@H](C)O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCNC(=O)CN2CCN(CC(=O)O)CCN(CC(=O)O)CCN(CC(=O)O)CC2)NC(=O)CNC(=O)[C@H](CC2CCNCC2)NC1=O] |
| 170 | 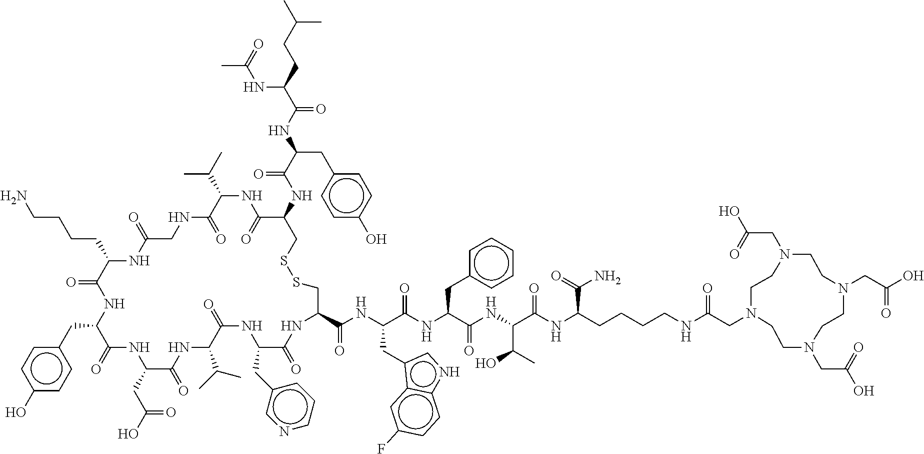 |
| 171 | 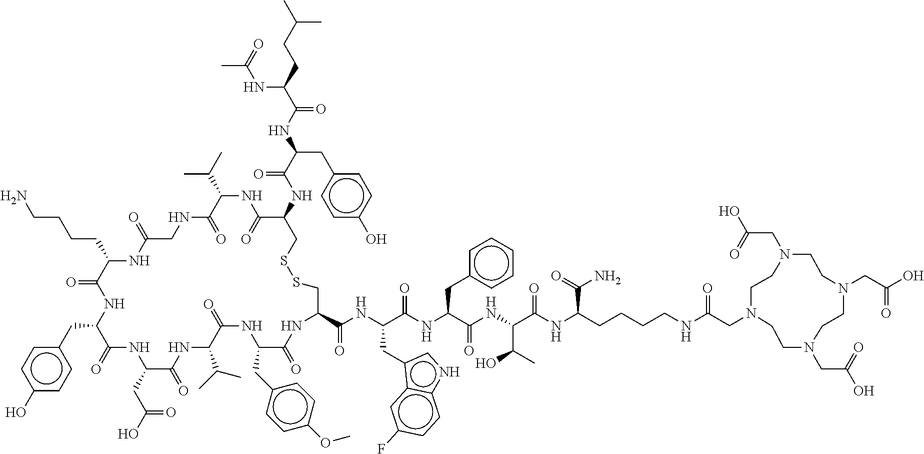 |
| 172 | 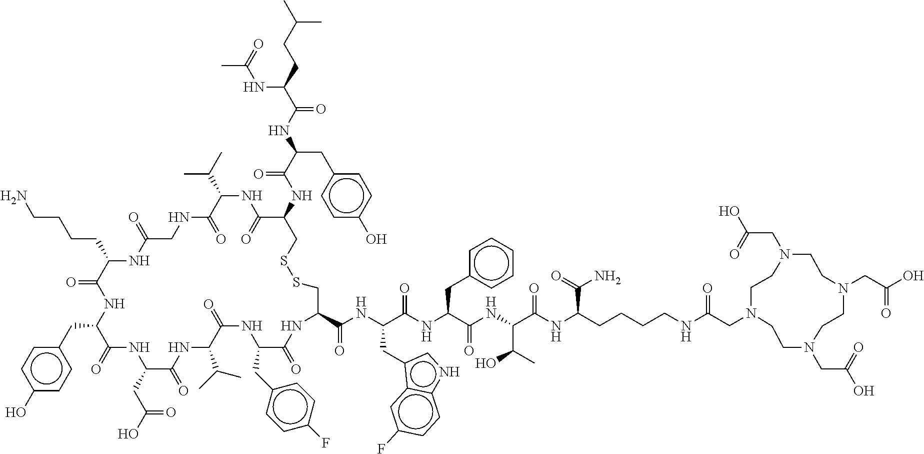 |
| 173 | 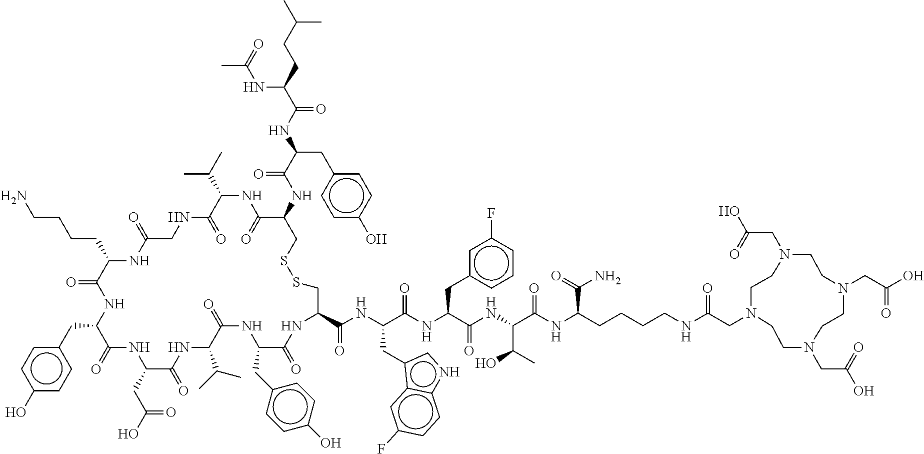 |
| 174 | 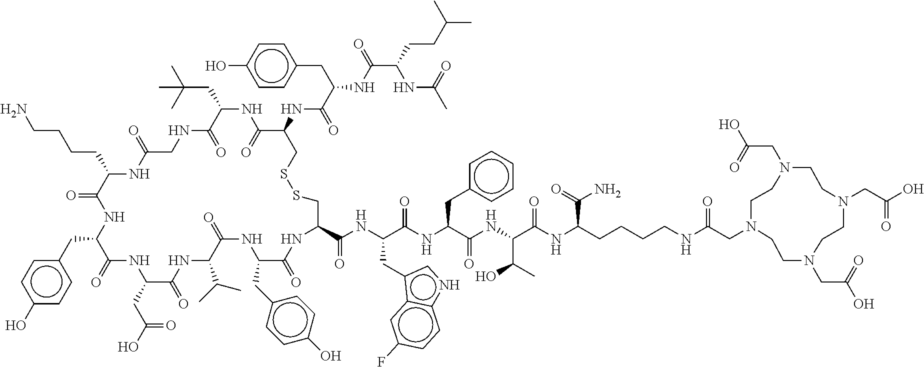 |
| 175 | 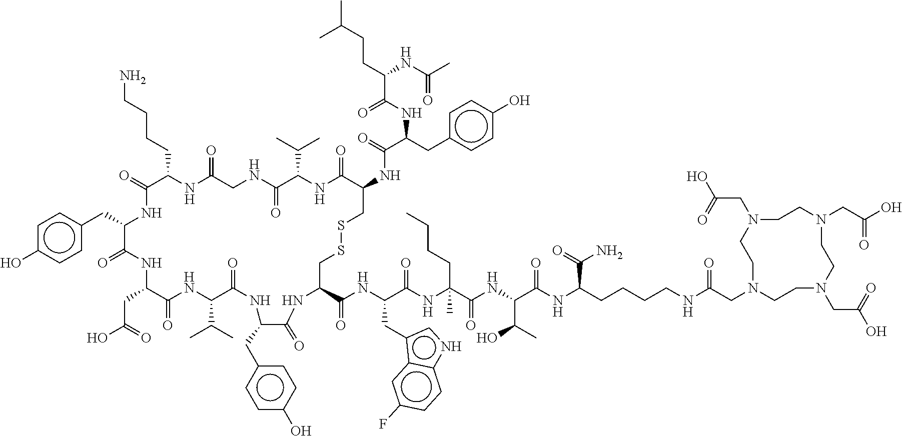 |
| 176 | 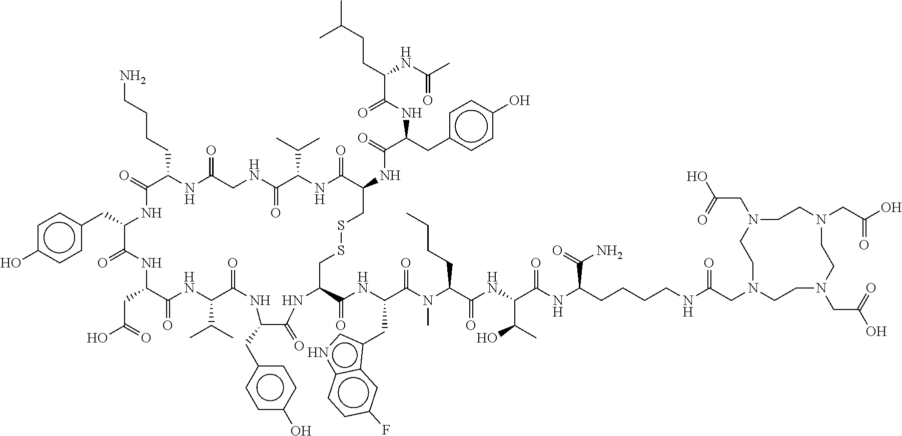 |
| 177 | 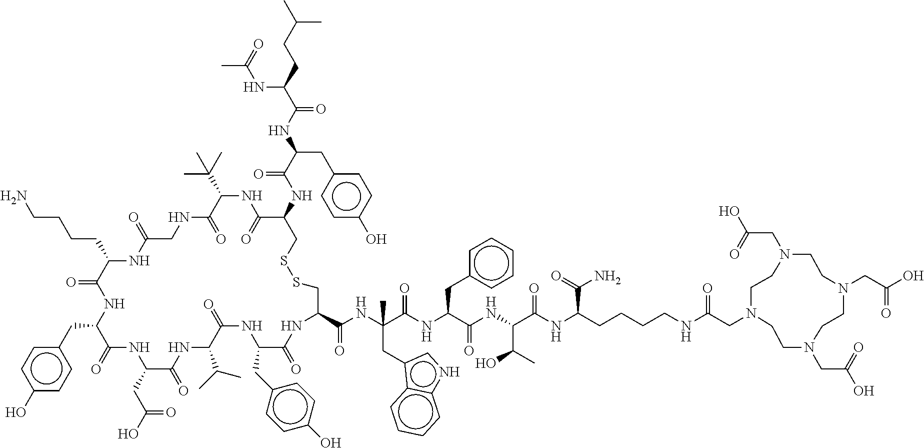 |
| 178 | 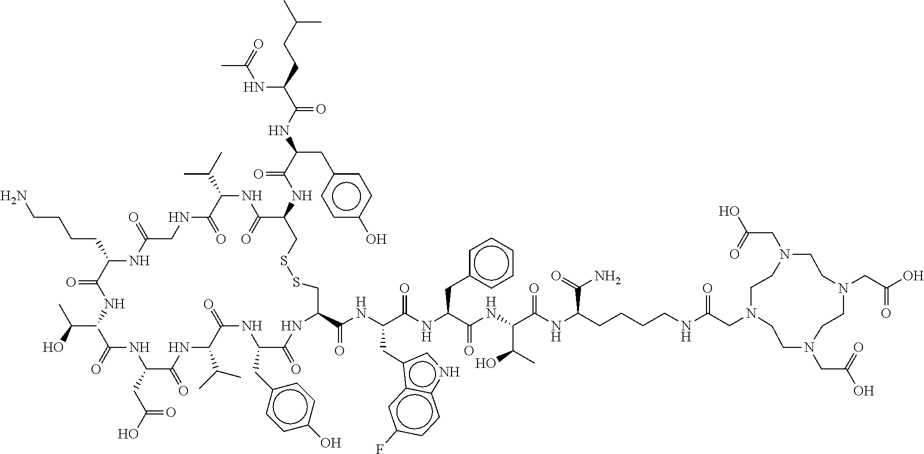 |
| 179 | 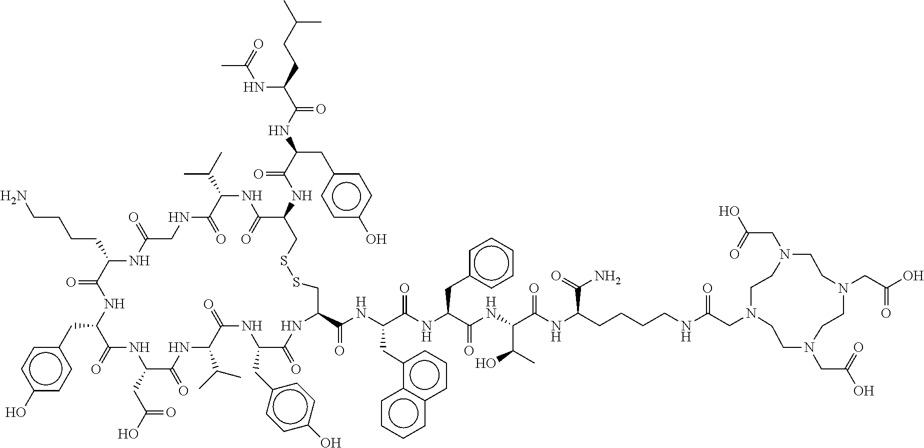 |
| 180 | 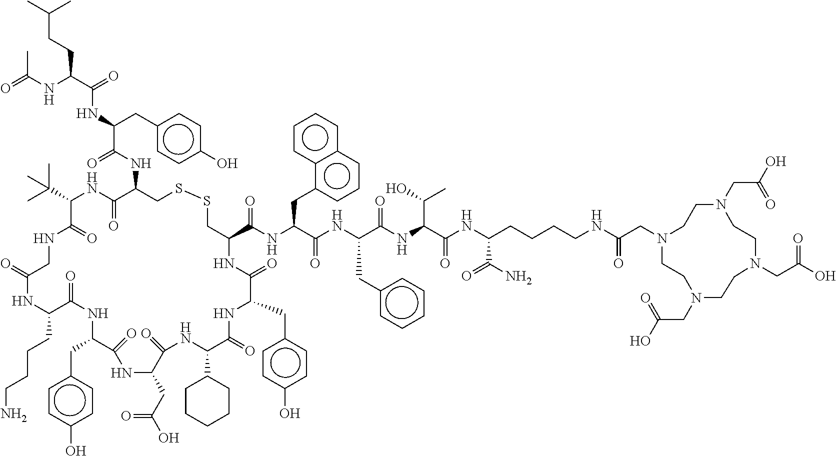 |
| 181 | 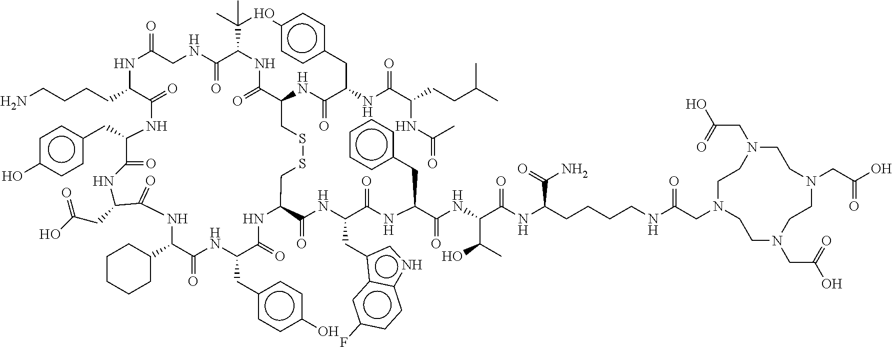 |
| 182 | 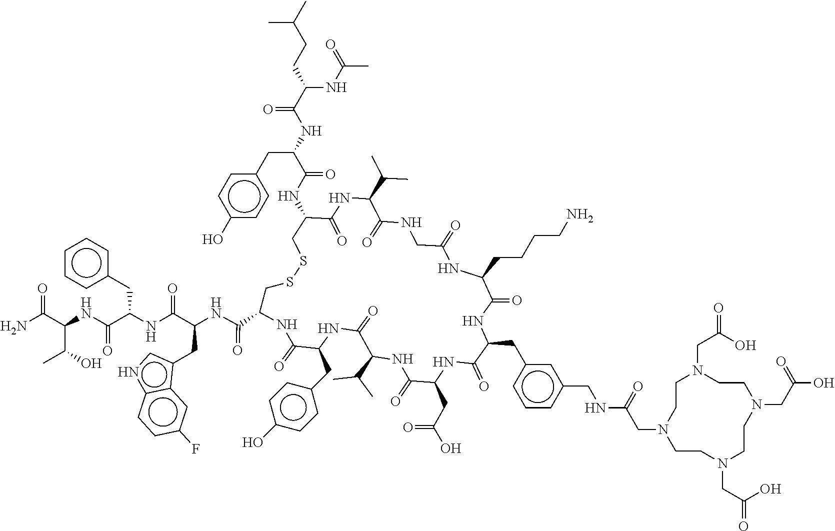 |
| 183 | 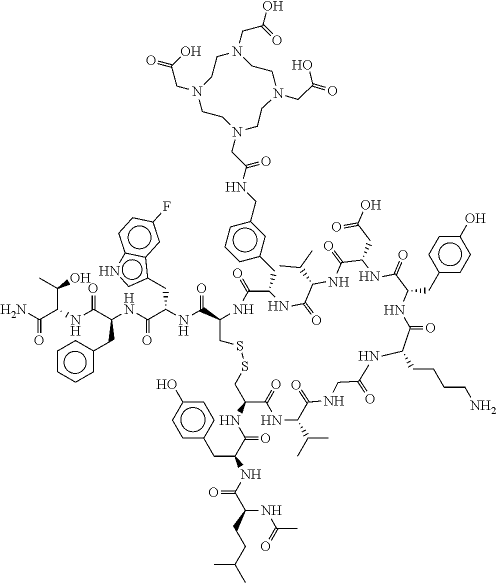 |
| 184 | 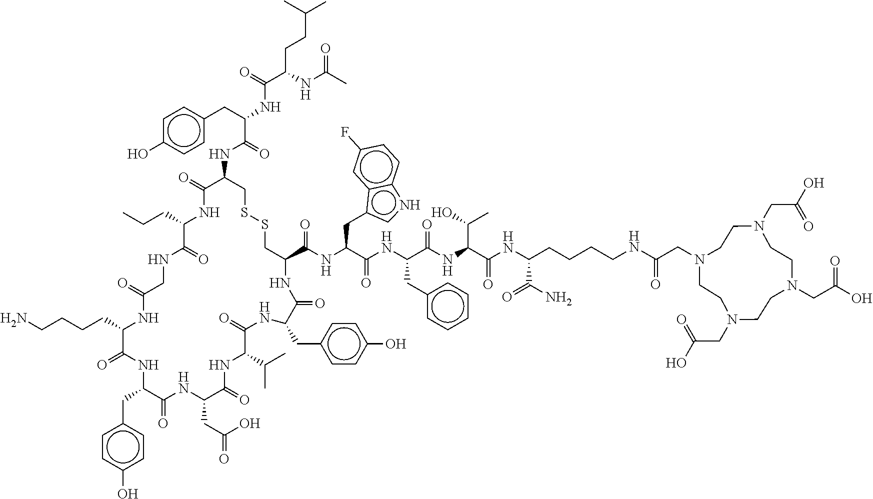 |
| 185 | 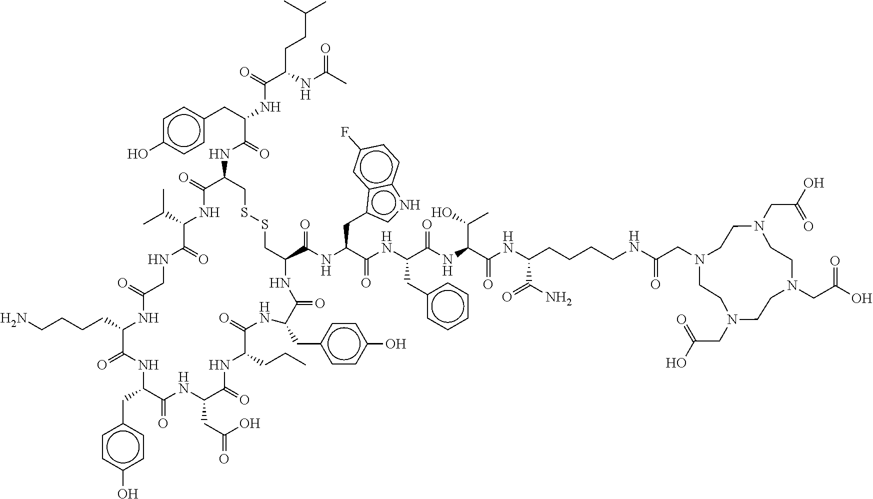 |
| 186 | 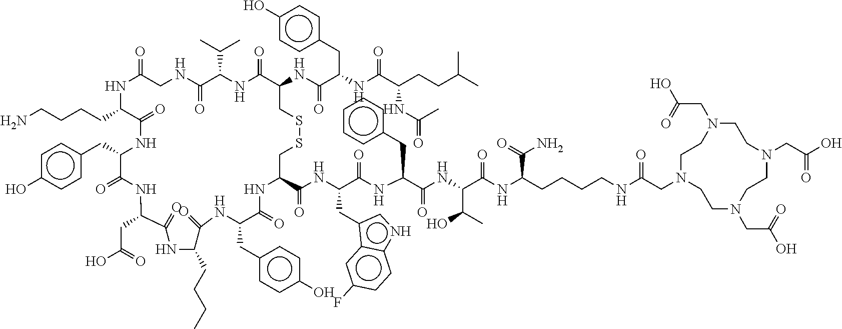 |
| 187 | 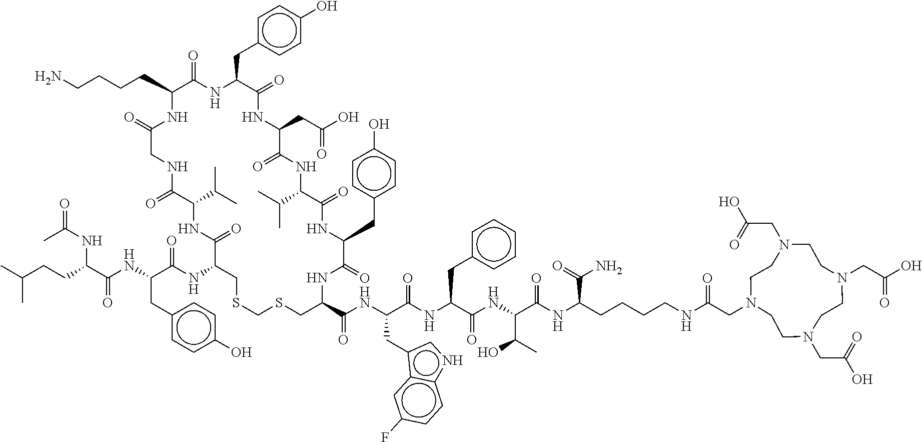 |
| 188 | 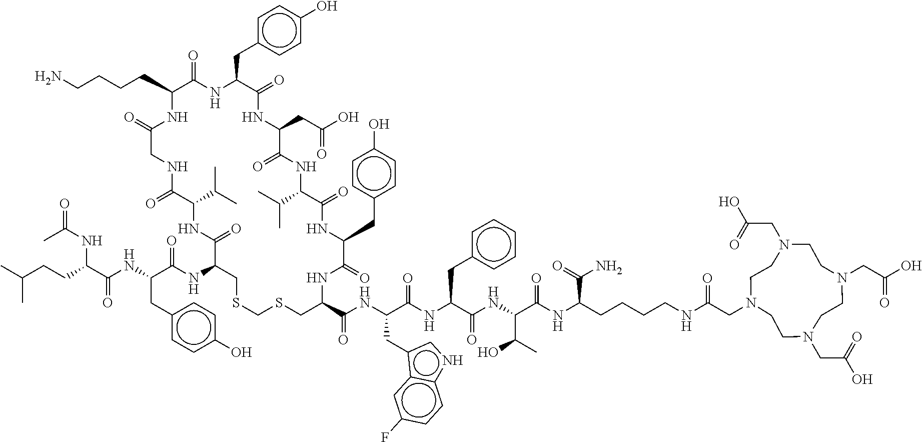 |
| 189 | 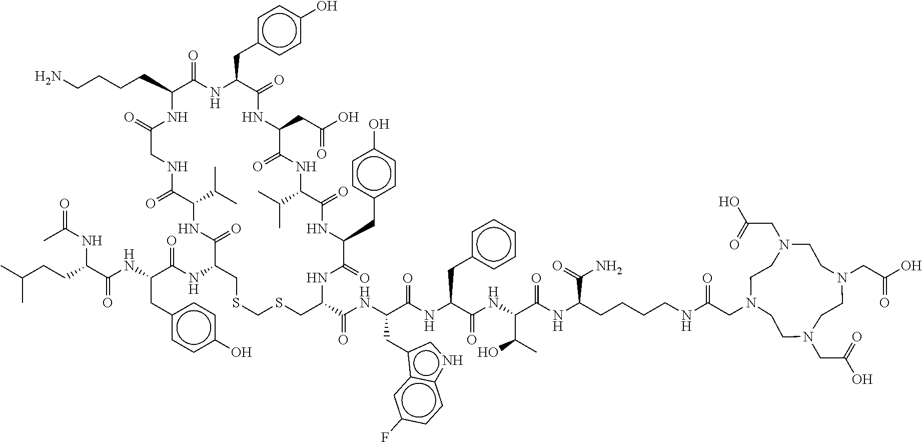 |
| 190 | 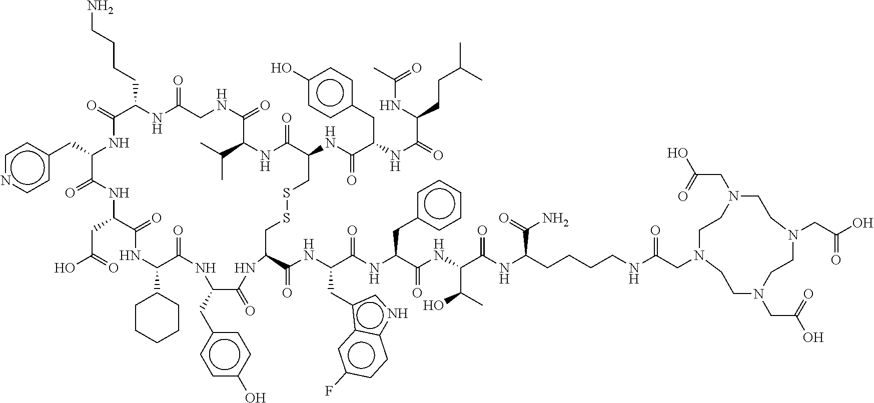 |
| 191 | 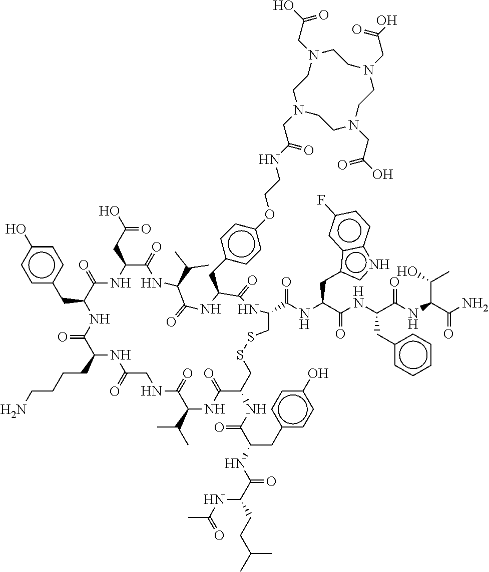 |
| 192 | 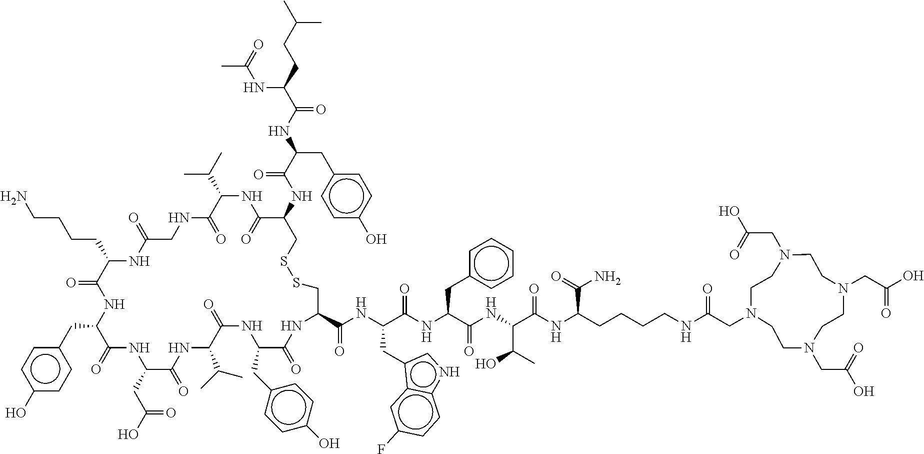 |
| 193 | 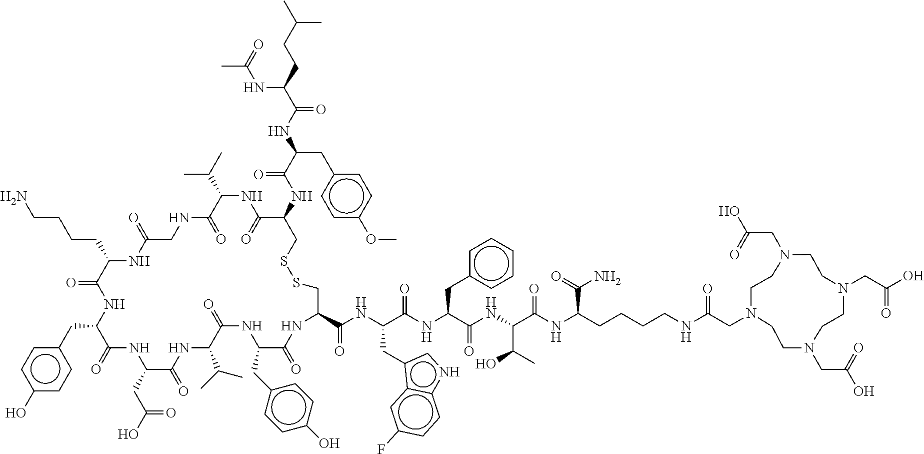 |
| 194 | 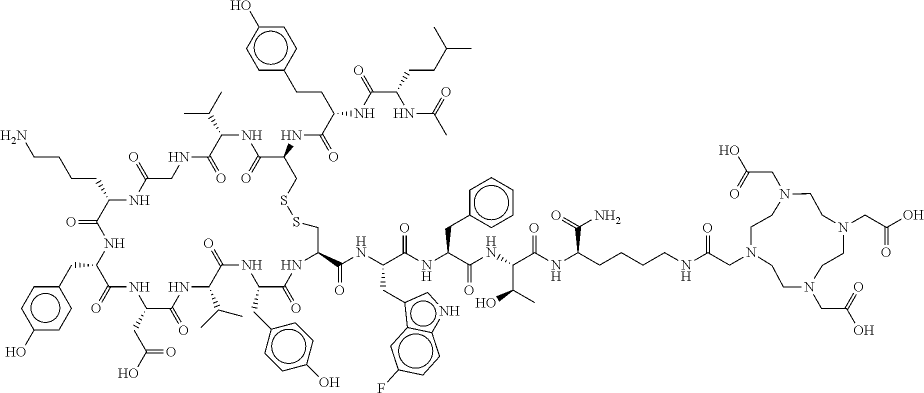 |
| 195 | 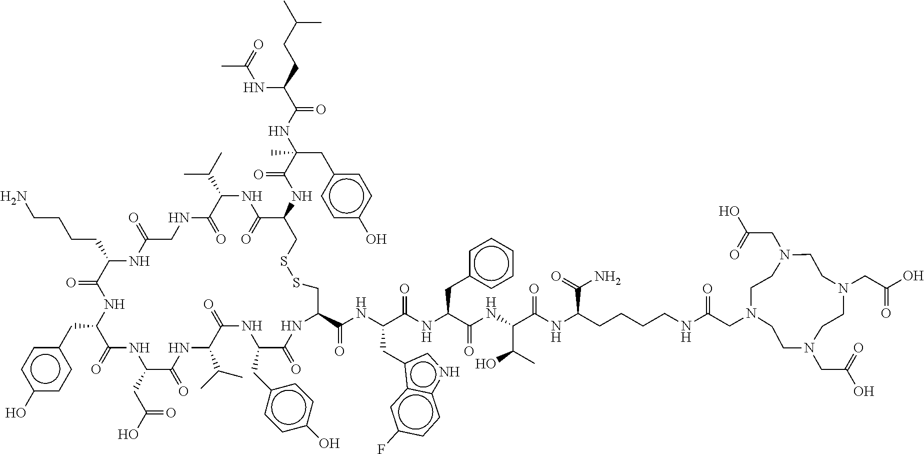 |
| 196 | 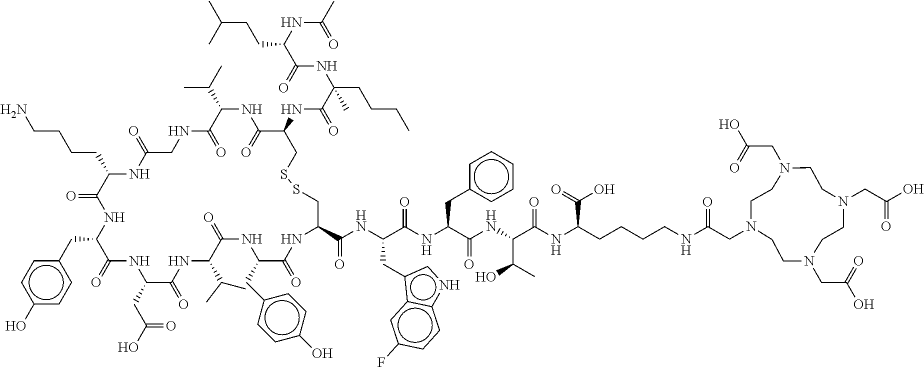 |
| 197 | 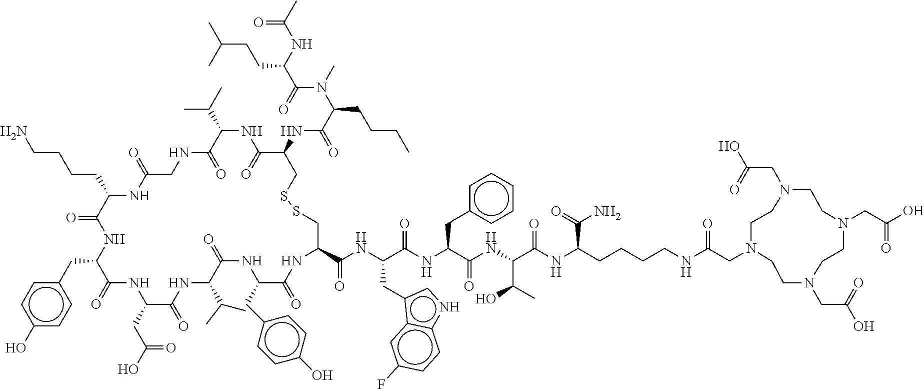 |
| 198 | 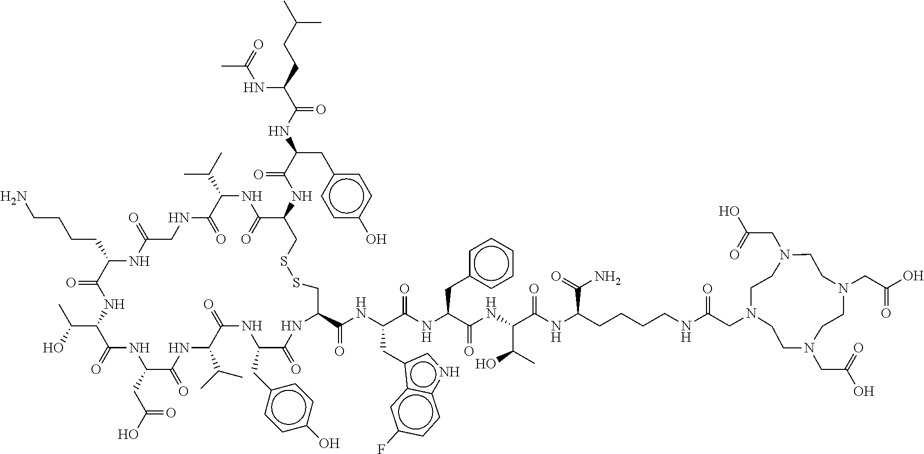 |
| 199 | 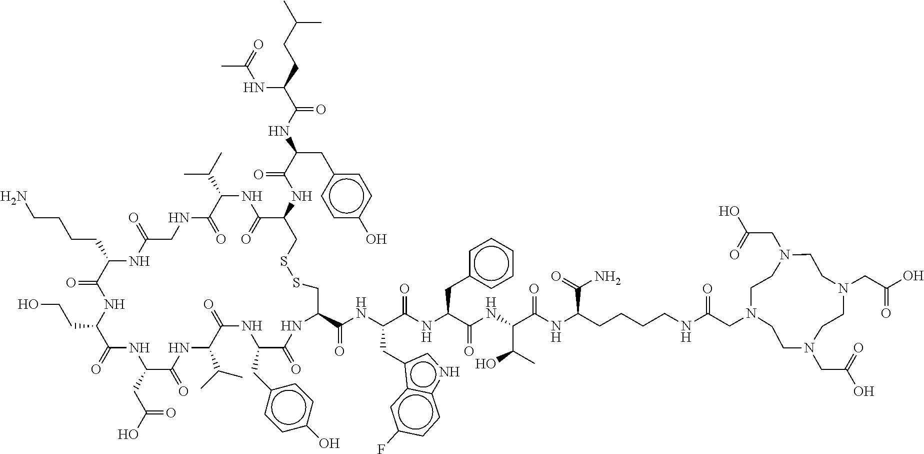 |
| 200 | 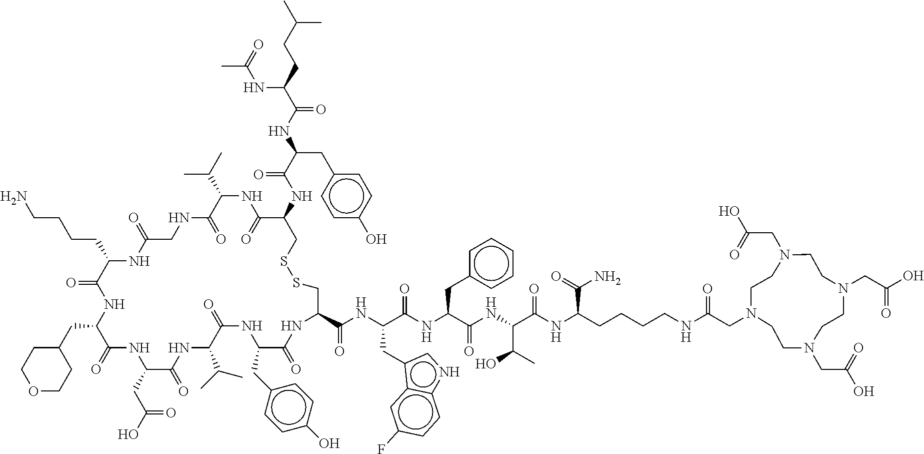 |
| 201 | 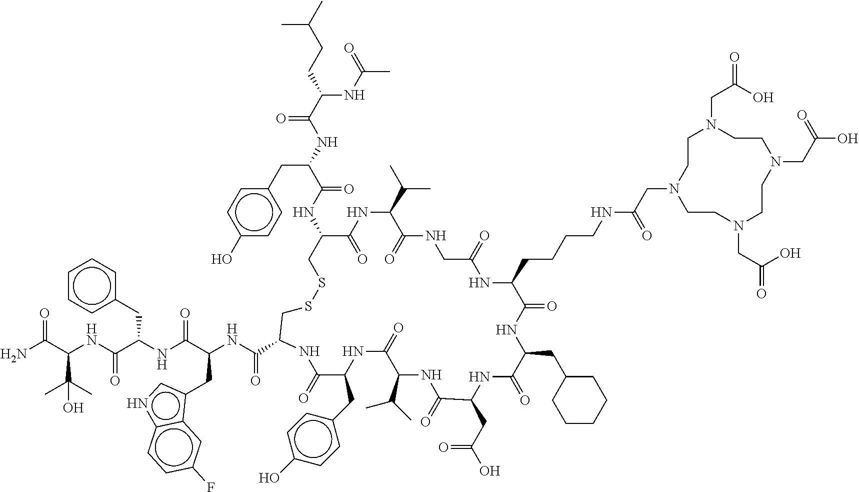 |
| 202 | 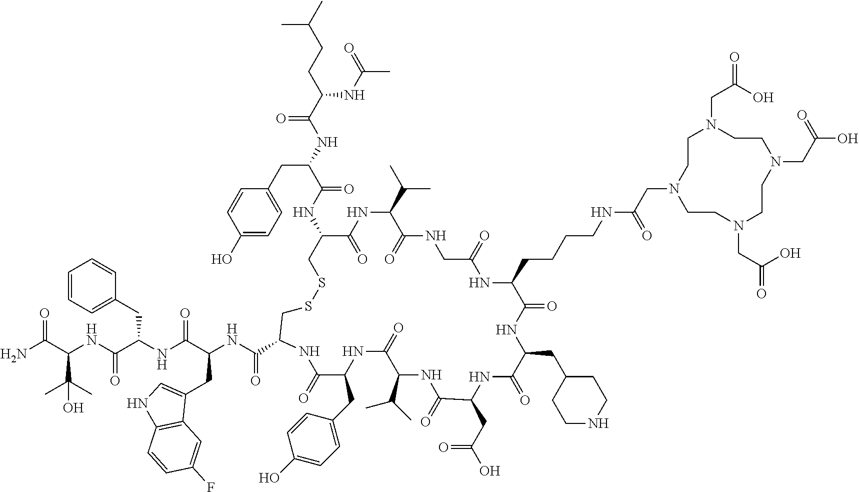 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@@H](Cc2ccccc2)C(=O)N[C@H](C(N)=O)C(C)(C)O)NC(=O)[C@H](Cc2ccc(O)cc2)NC(=O)[C@H](C(C)C)NC(=O)[C@H](CC(=O)O)NC(=O)[C@H](CC2CCNCC2)NC(=O)[C@H](CCCCNC(=O)CN2CCN(CC(=O)O)CCN(CC(=O)O)CCN(CC(=O)O)CC2)NC(=O)CNC(=O)[C@H](C(C)C)NC1=O] |
| 203 | 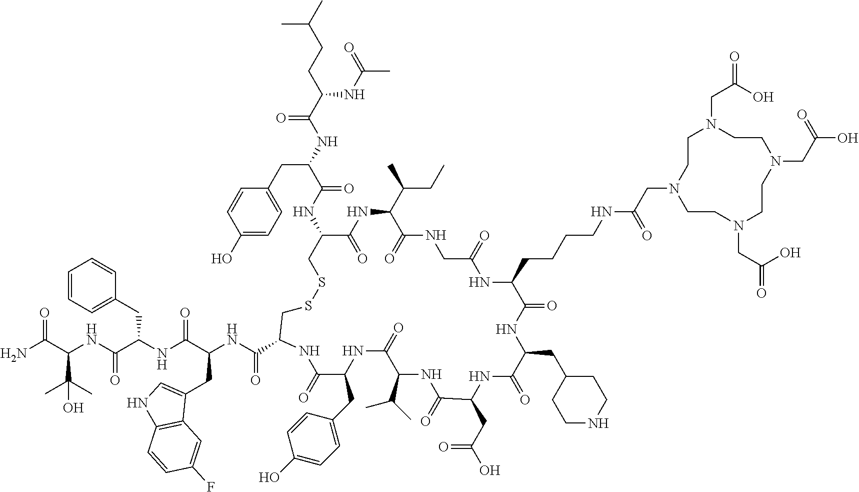 [SMILES: CC[C@H](C)[C@@H]1NC(=O)[C@@H](NC(=O)[C@H](Cc2ccc(O)cc2)NC(=O)[C@H](CCC(C)C)NC(C)=O)CSSC[C@@H](C(=O)N[C@@H](Cc2c[nH]c3ccc(F)cc23)C(=O)N[C@@H](Cc2ccccc2)C(=O)N[C@H](C(N)=O)C(C)(C)O)NC(=O)[C@H](Cc2ccc(O)cc2)NC(=O)[C@H](C(C)C)NC(=O)[C@H](CC(=O)O)NC(=O)[C@H](CC2CCNCC2)NC(=O)[C@H](CCCCNC(=O)CN2CCN(CC(=O)O)CCN(CC(=O)O)CCN(CC(=O)O)CC2)NC(=O)CNC1=O] |
| 204 | 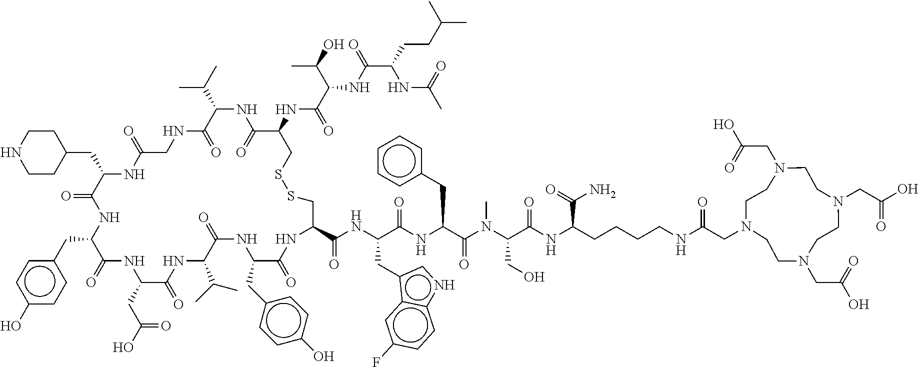 |
| 205 | 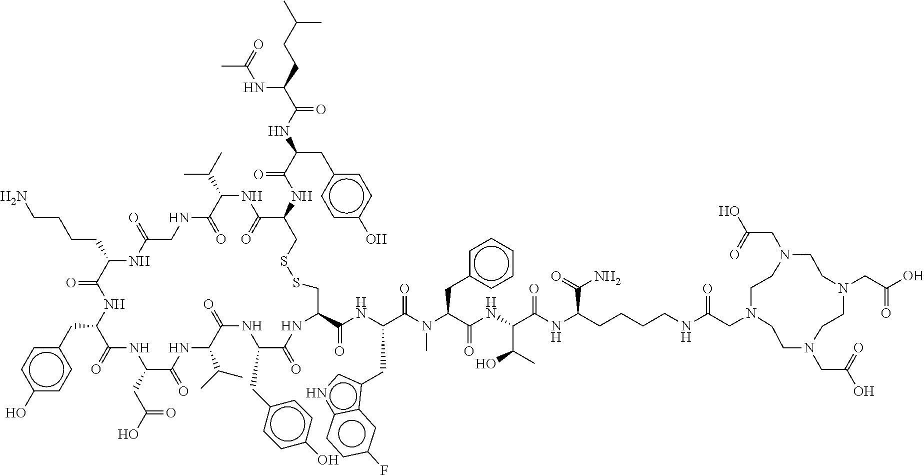 |
| 206 | 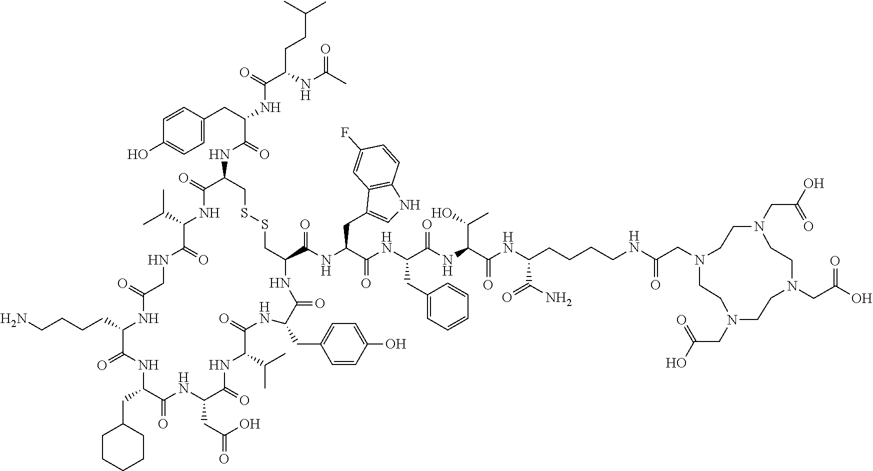 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@@H](Cc2ccccc2)C(=O)N[C@H](C(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](Cc2ccc(O)cc2)NC(=O)[C@H](C(C)C)NC(=O)[C@H](CC(=O)O)NC(=O)[C@H](CC2CCCCC2)NC(=O)[C@H](CCCCN)NC(=O)CNC(=O)[C@H](C(C)C)NC1=O] |
| 207 | 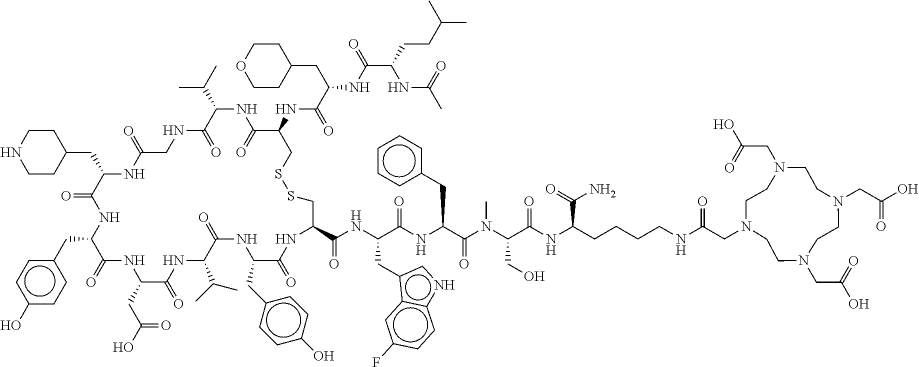 |
| 208 | 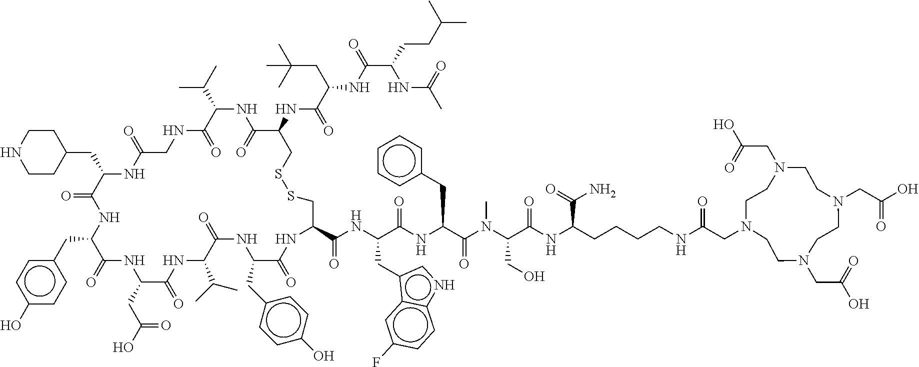 |
| 209 | 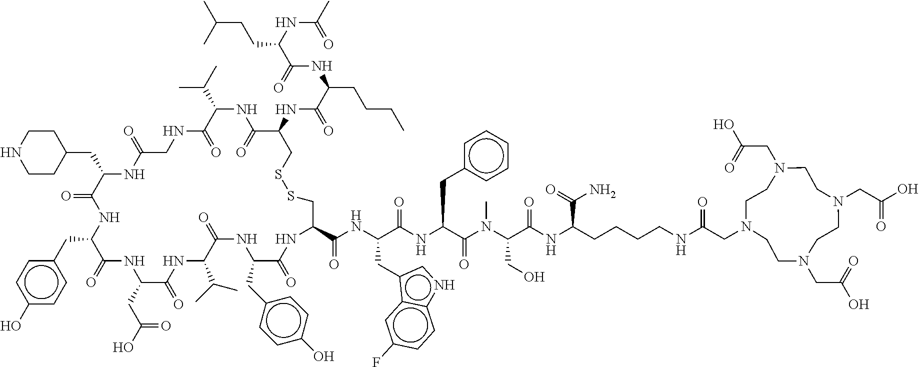 |
| 210 | 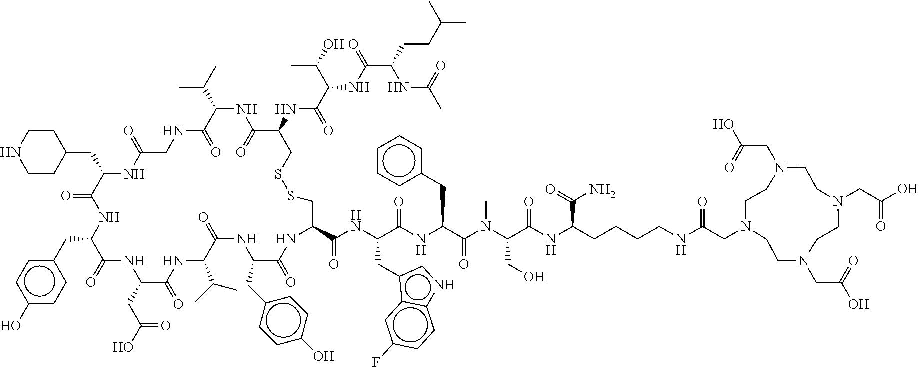 |
| 211 | 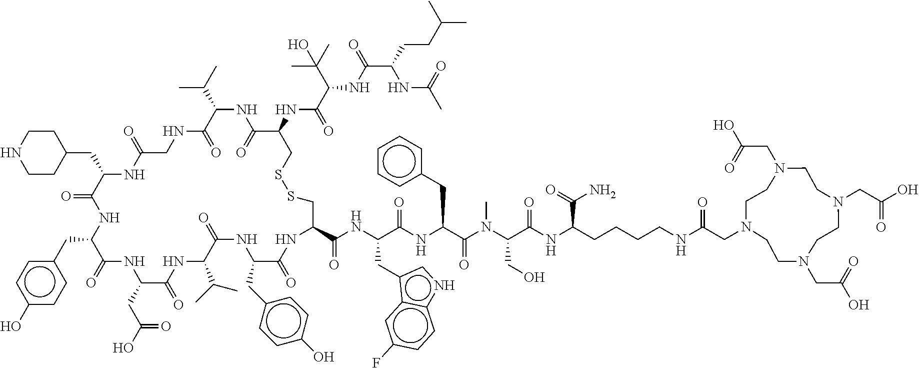 |
| 212 | 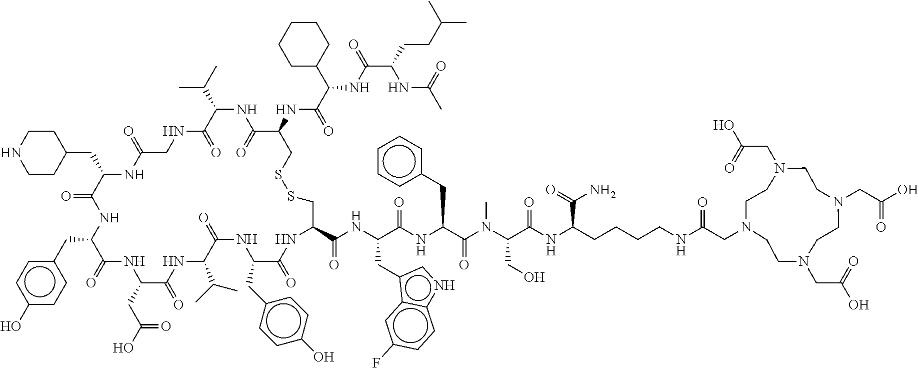 |
| 213 | 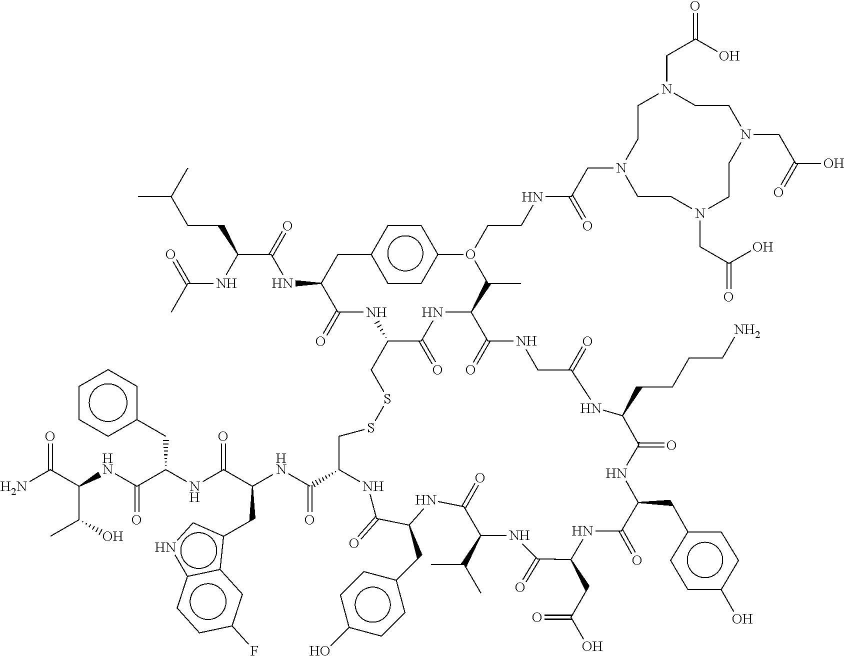 |
| 214 | 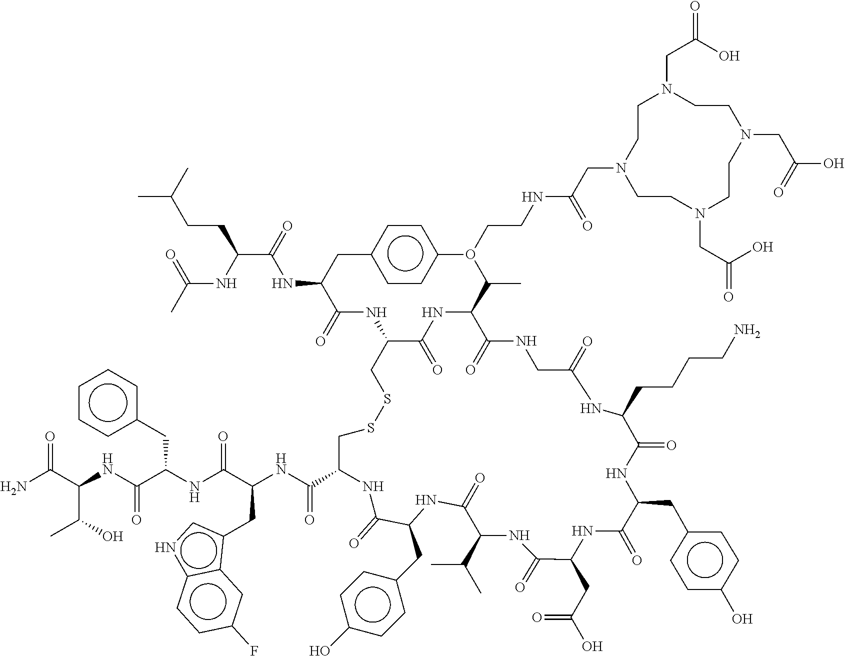 |
| 215 | 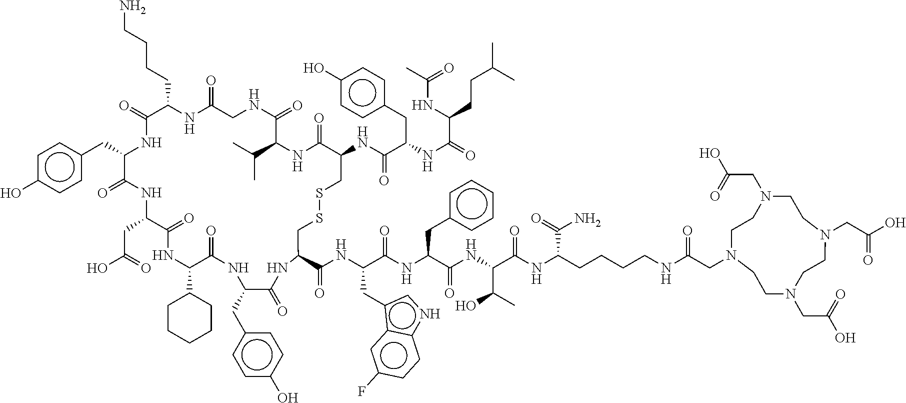 |
| 216 | 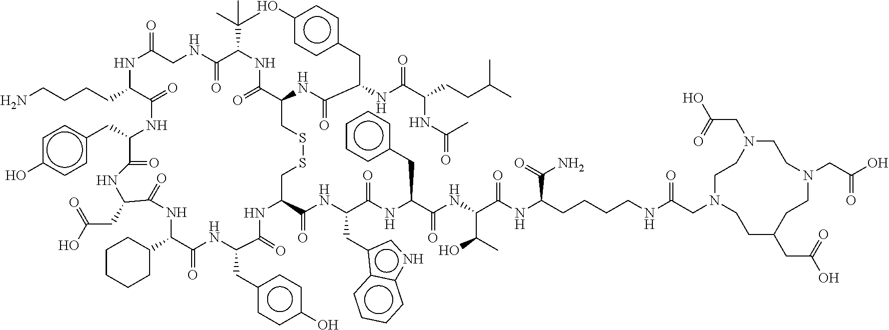 |
| 217 | 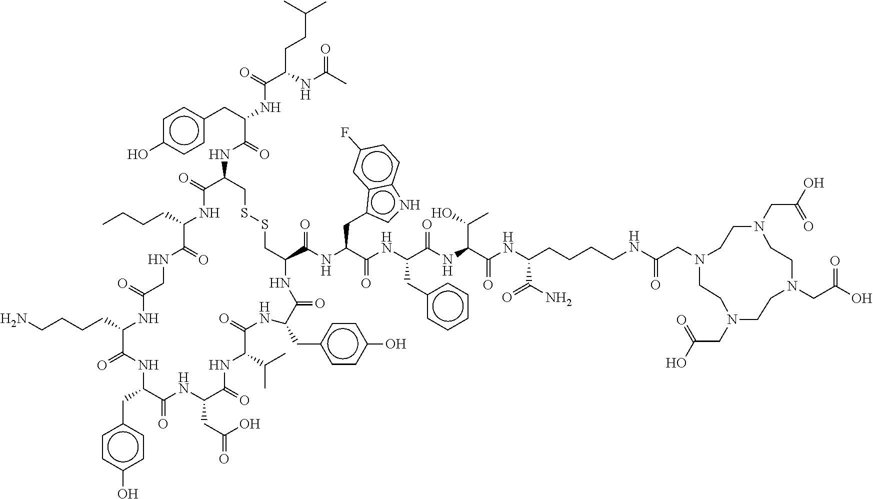 |
| 218 | 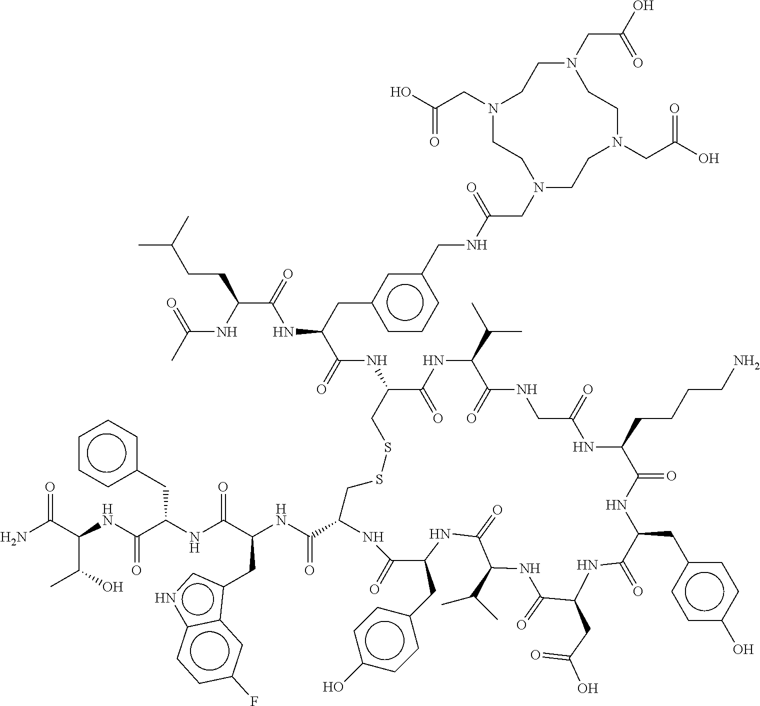 |
| 219 | 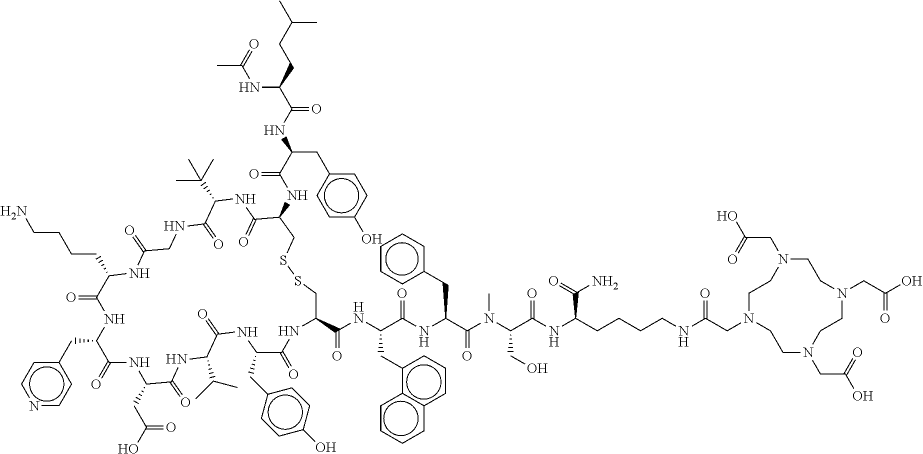 |
| 220 | 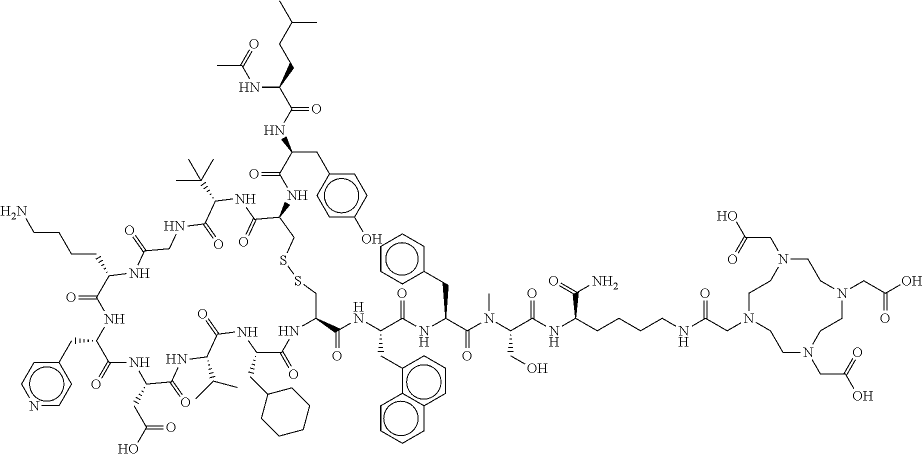 |
| 221 | 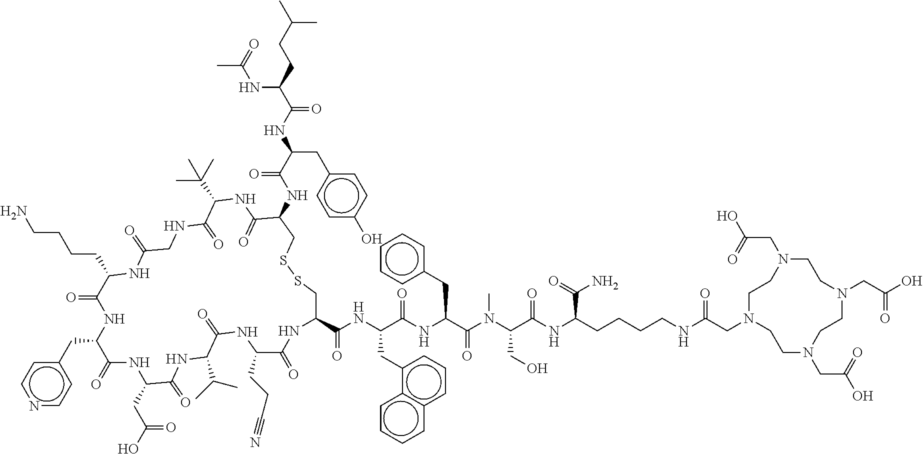 |
| 222 | 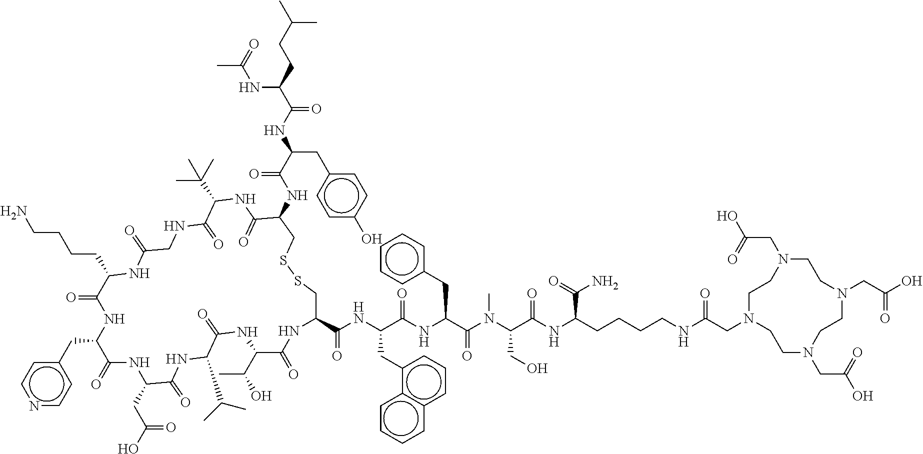 |
| 223 | 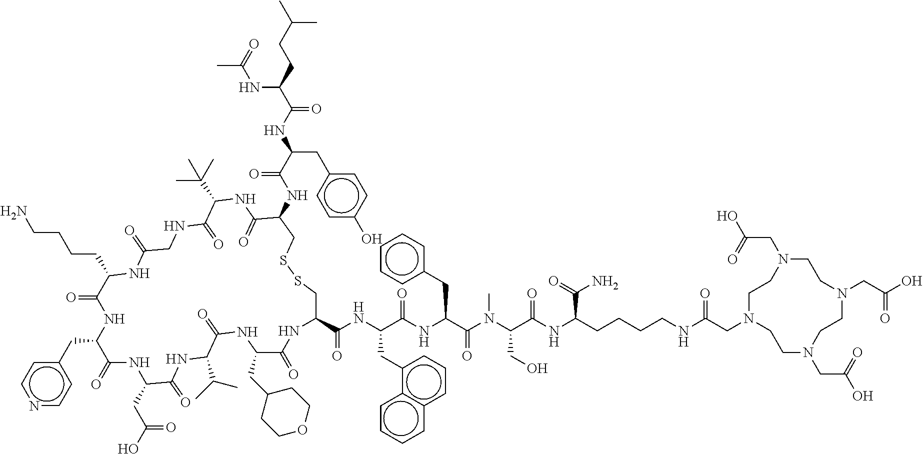 |
| 224 | 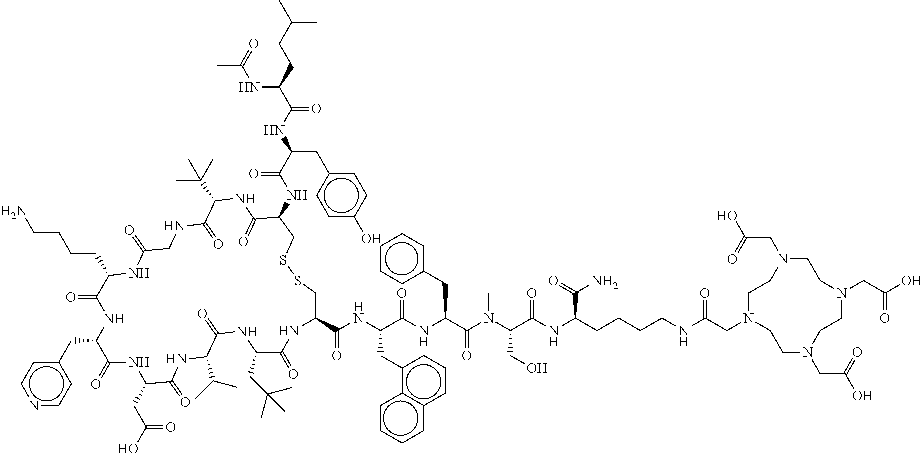 |
| 225 | 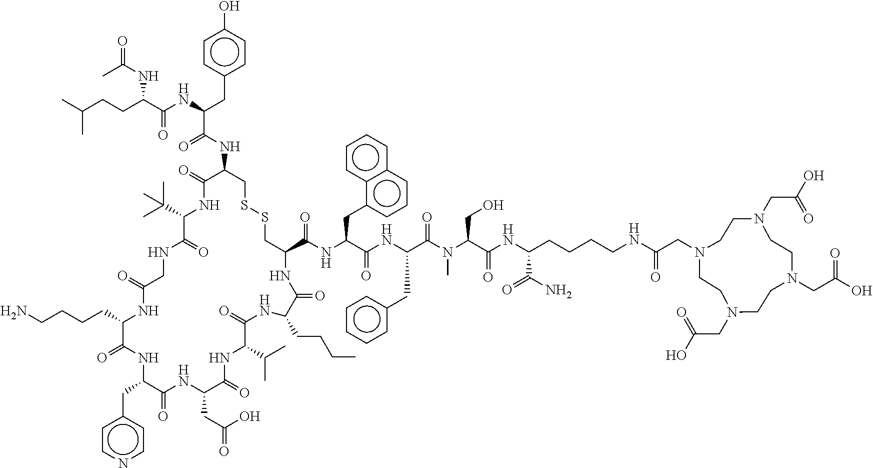 |
| 226 | 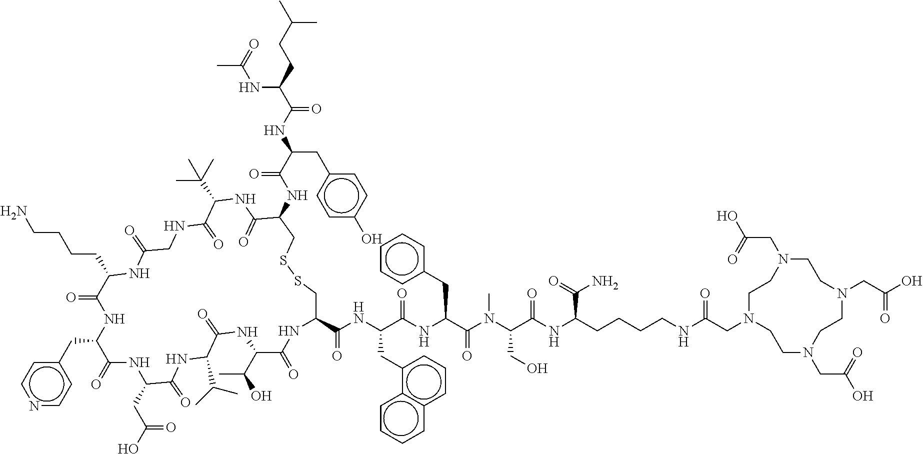 |
| 227 | 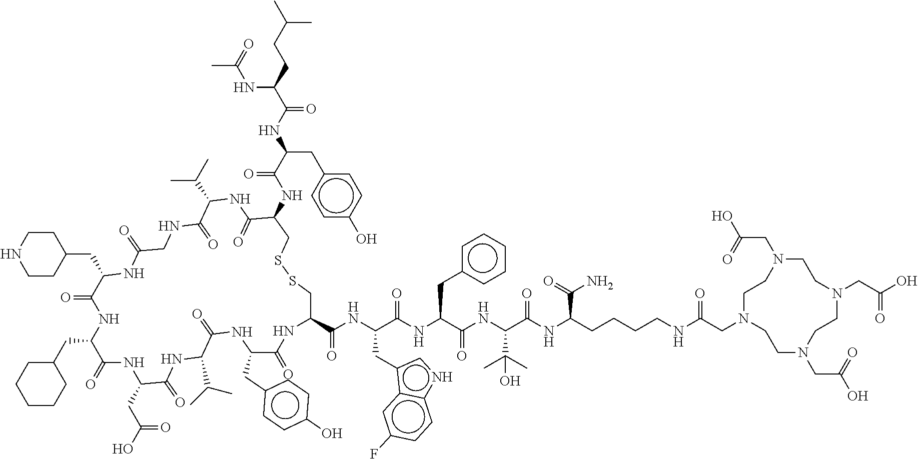 |
| 228 | 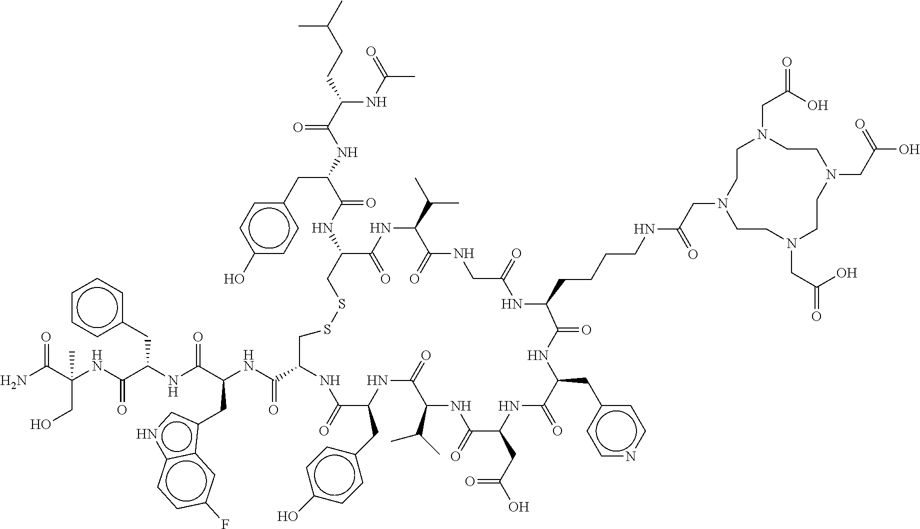 |
| 229 | 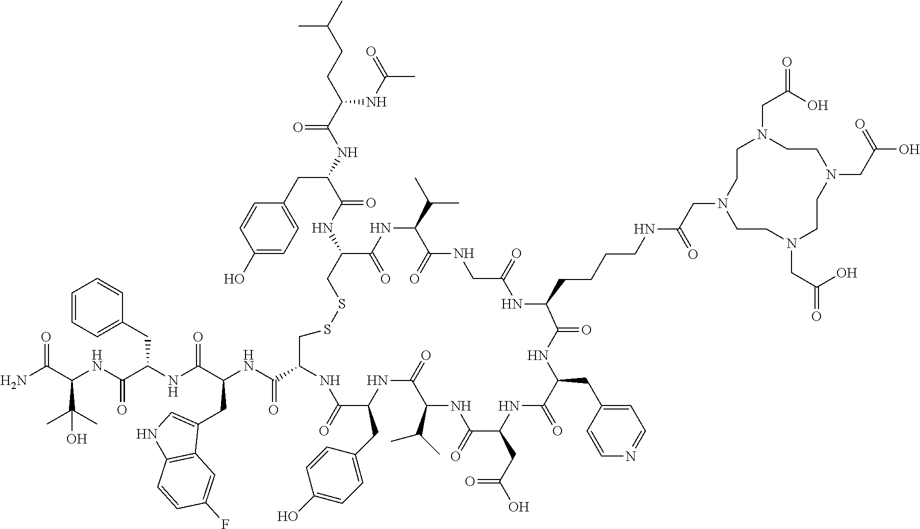 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@@H](Cc2ccccc2)C(=O)N[C@H](C(N)=O)C(C)(C)O)NC(=O)[C@H](Cc2ccc(O)cc2)NC(=O)[C@H](C(C)C)NC(=O)[C@H](CC(=O)O)NC(=O)[C@H](Cc2ccncc2)NC(=O)[C@H](CCCCNC(=O)CN2CCN(CC(=O)O)CCN(CC(=O)O)CCN(CC(=O)O)CC2)NC(=O)CNC(=O)[C@H](C(C)C)NC1=O] |
| 230 | 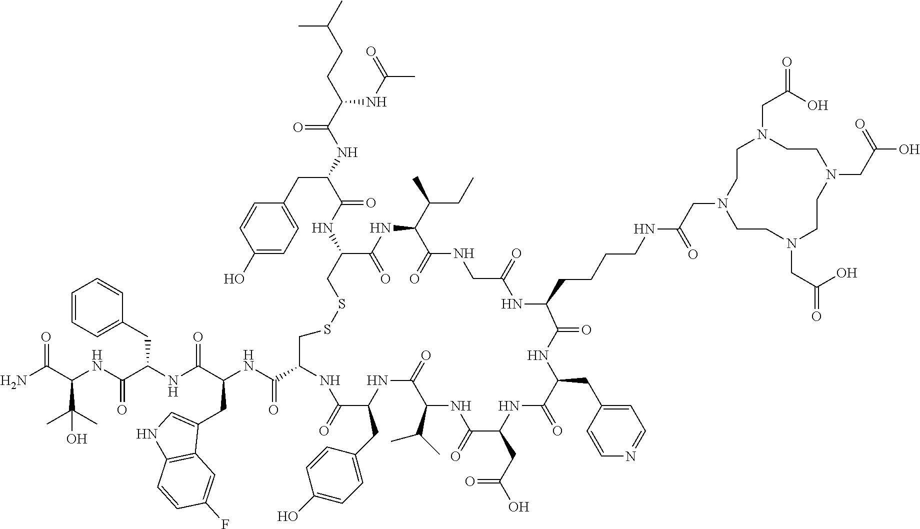 [SMILES: CC[C@H](C)[C@@H]1NC(=O)[C@@H](NC(=O)[C@H](Cc2ccc(O)cc2)NC(=O)[C@H](CCC(C)C)NC(C)=O)CSSC[C@@H](C(=O)N[C@@H](Cc2c[nH]c3ccc(F)cc23)C(=O)N[C@@H](Cc2ccccc2)C(=O)N[C@H](C(N)=O)C(C)(C)O)NC(=O)[C@H](Cc2ccc(O)cc2)NC(=O)[C@H](C(C)C)NC(=O)[C@H](CC(=O)O)NC(=O)[C@H](Cc2ccncc2)NC(=O)[C@H](CCCCNC(=O)CN2CCN(CC(=O)O)CCN(CC(=O)O)CCN(CC(=O)O)CC2)NC(=O)CNC1=O] |
| 231 | 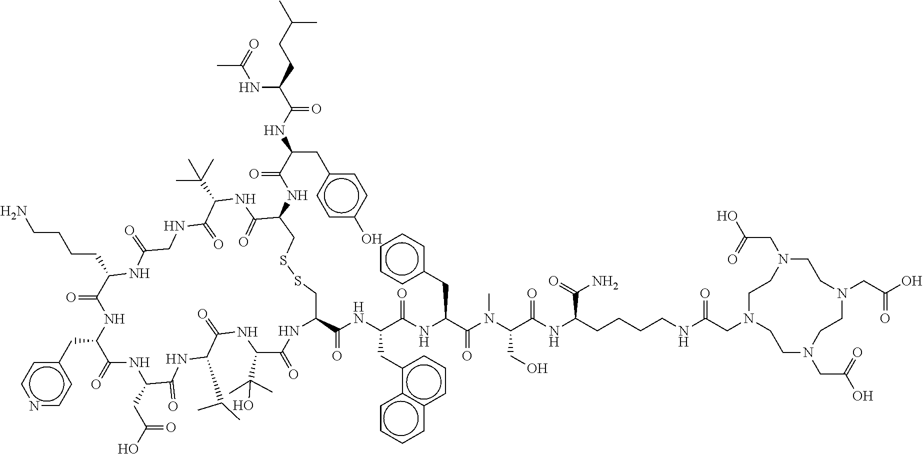 |
| 232 | 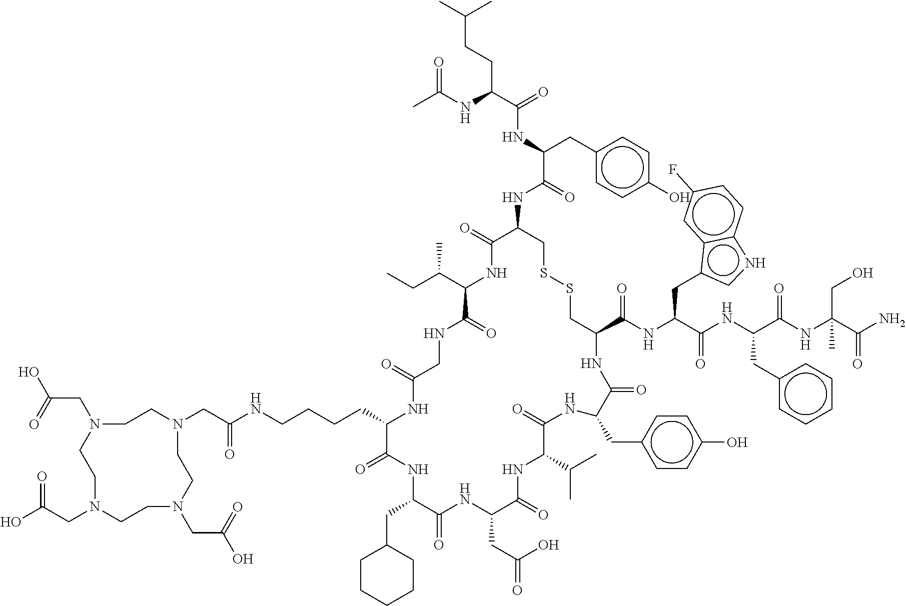 |
| 233 | 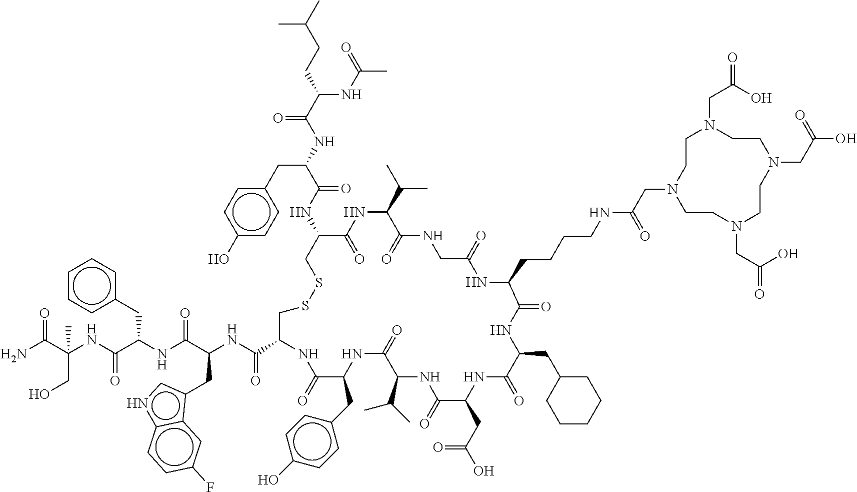 |
| 234 | 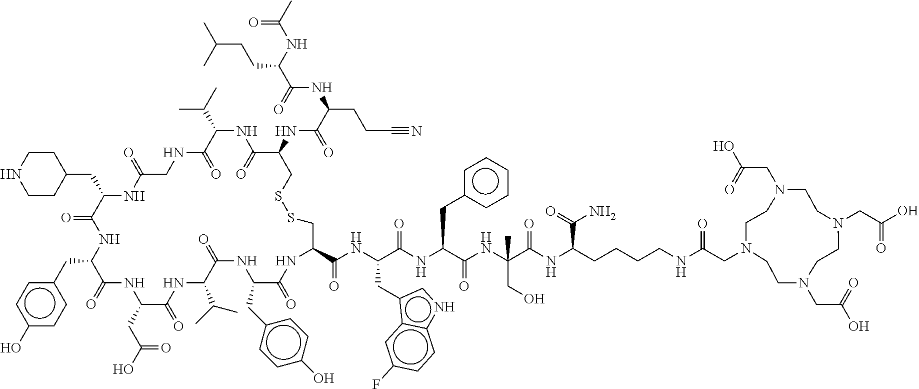 |
| 235 | 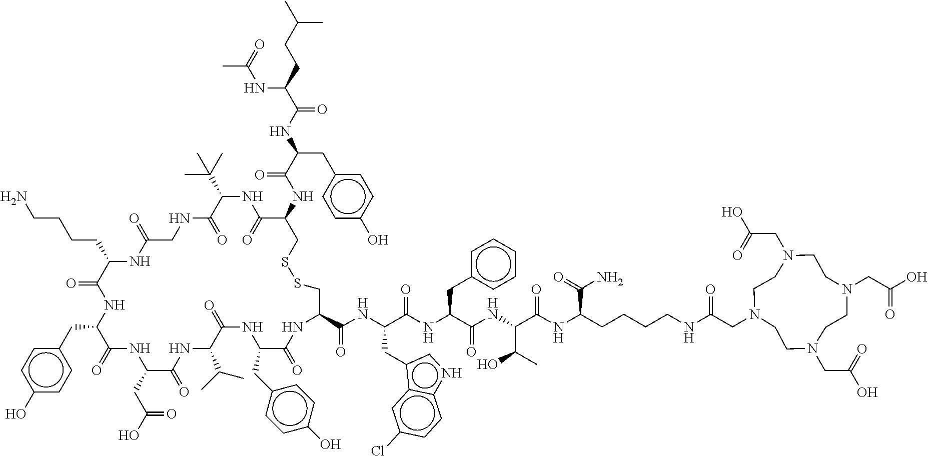 |
| 236 | 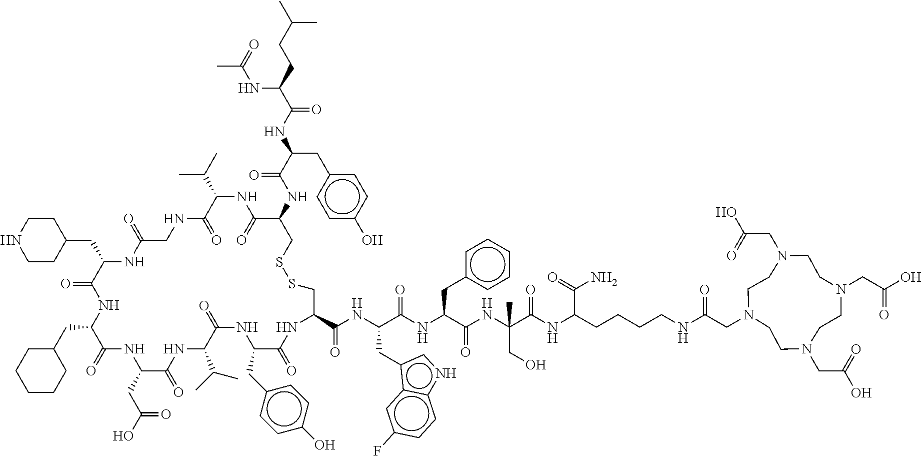 |
| 237 | 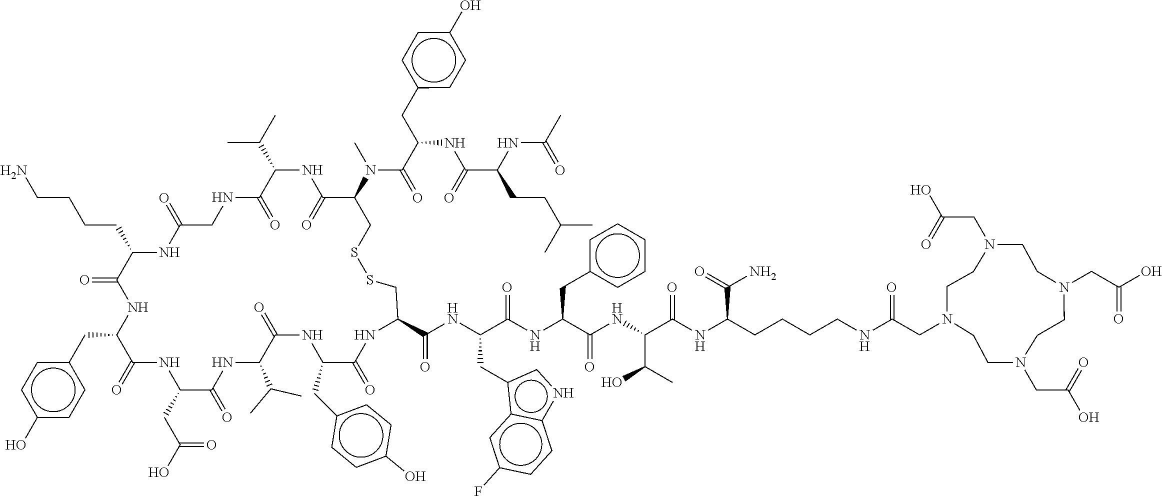 |
| 238 | 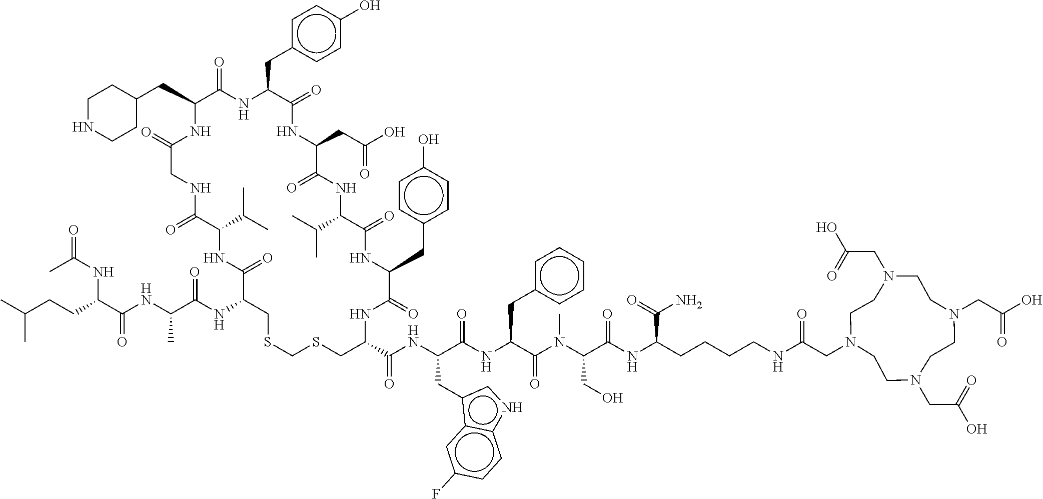 |
| 239 | 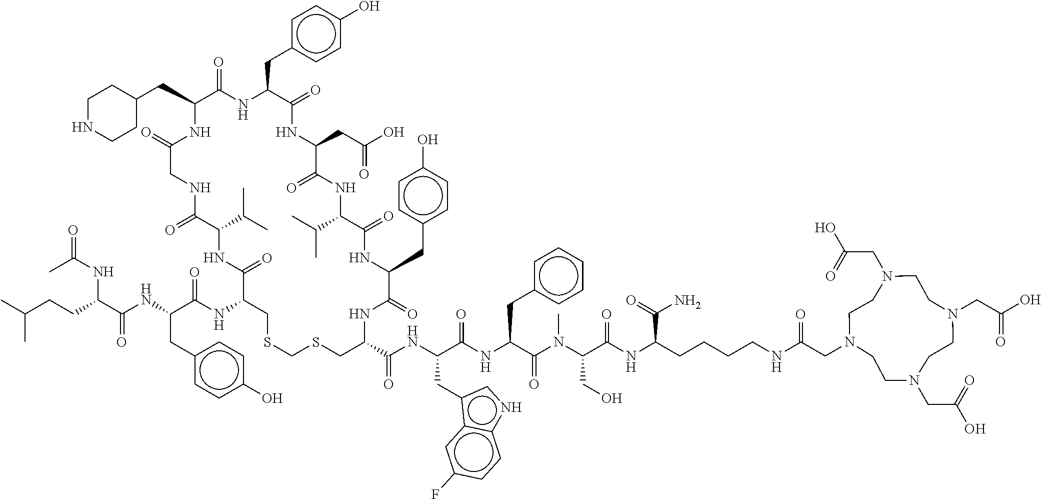 |
| 240 | 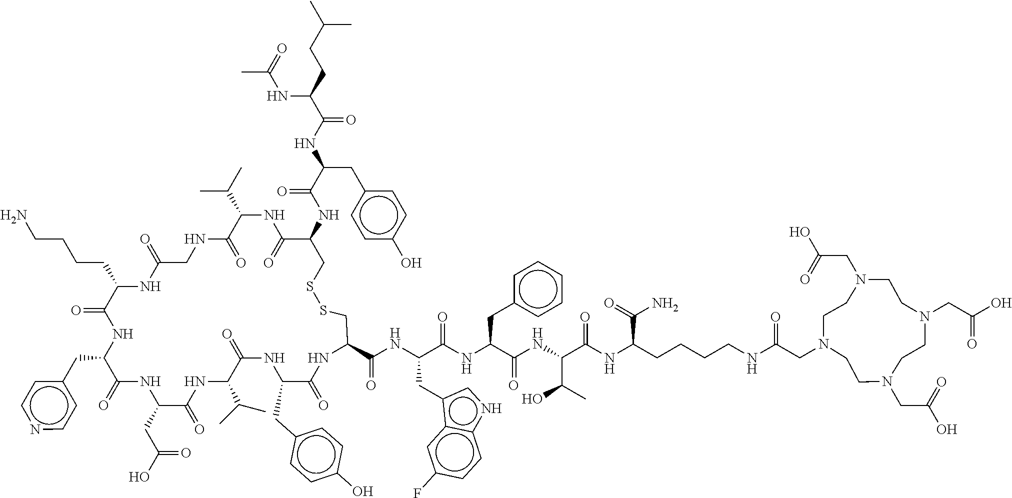 |
| 241 | 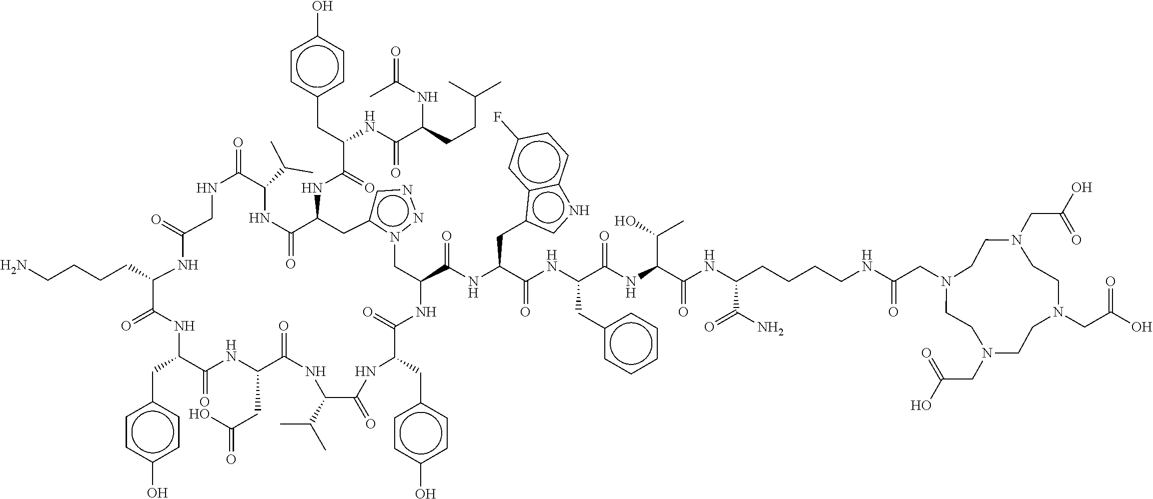 |
| 242 | 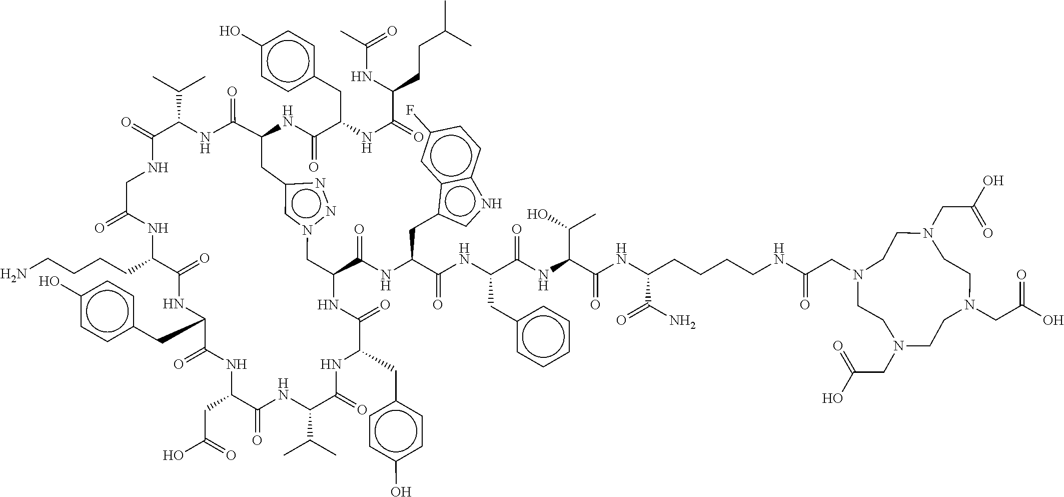 |
| 243 | 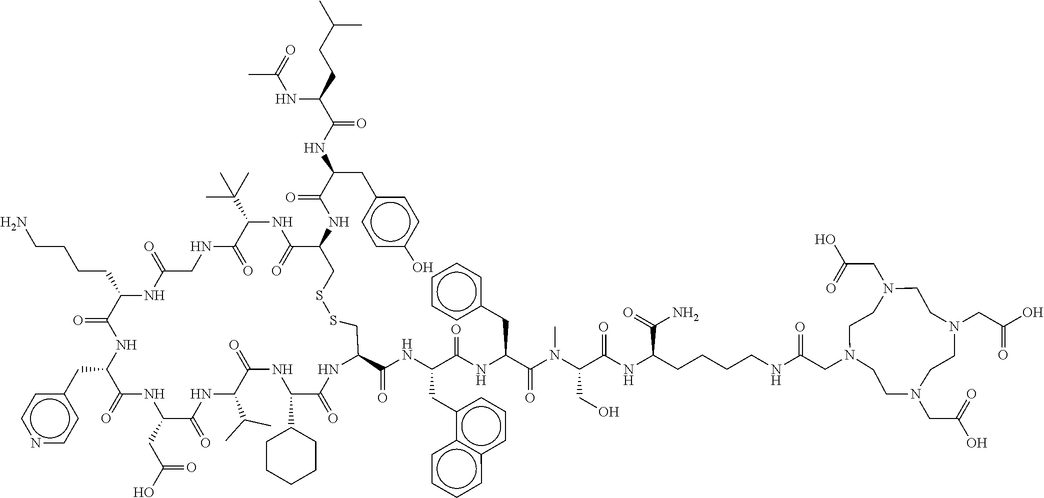 |
| 244 | 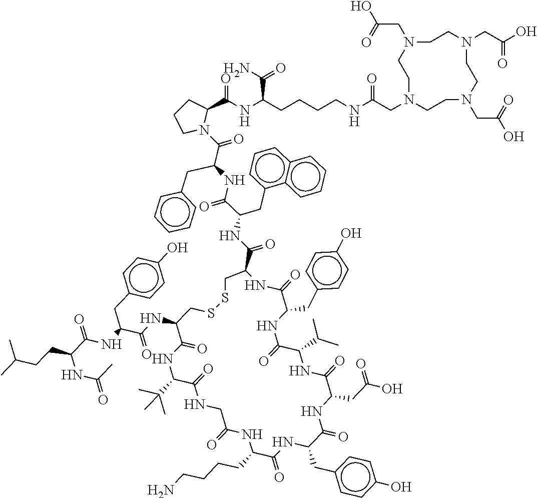 |
| 245 | 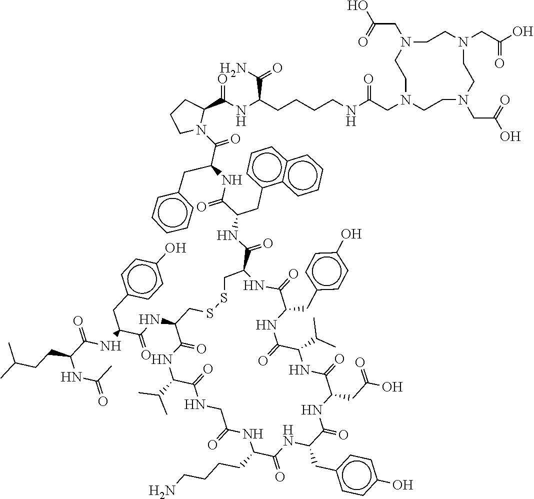 |
| 246 | 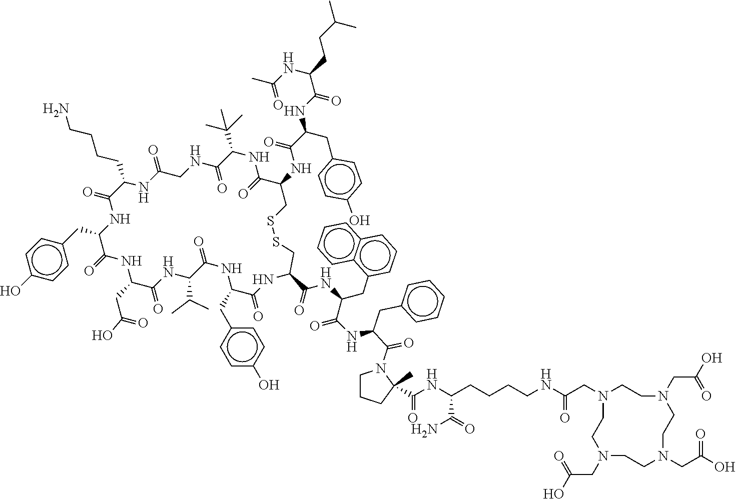 |
| 247 | 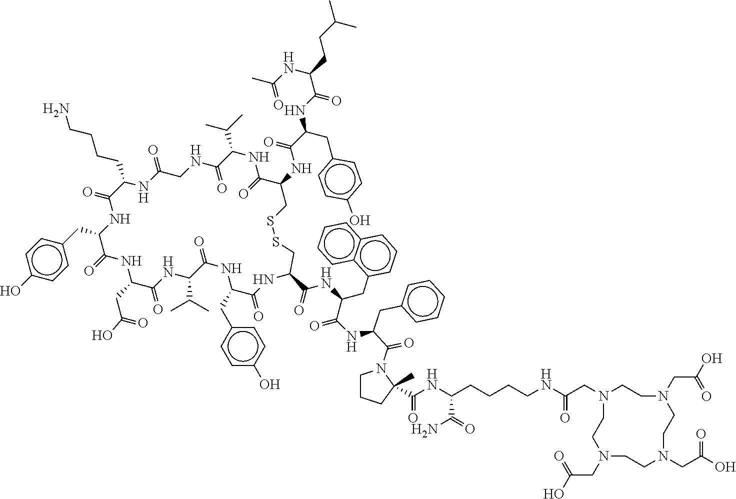 |
| 248 | 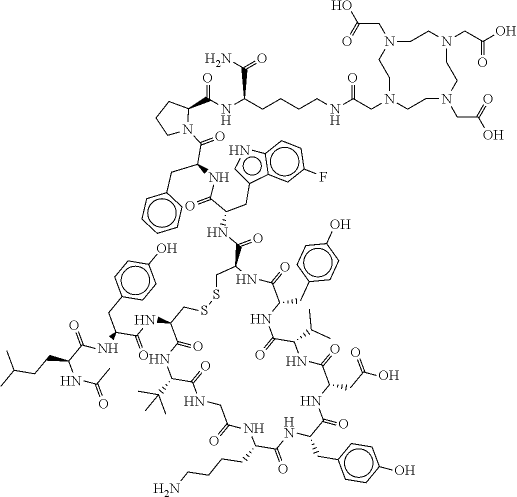 |
| 249 | 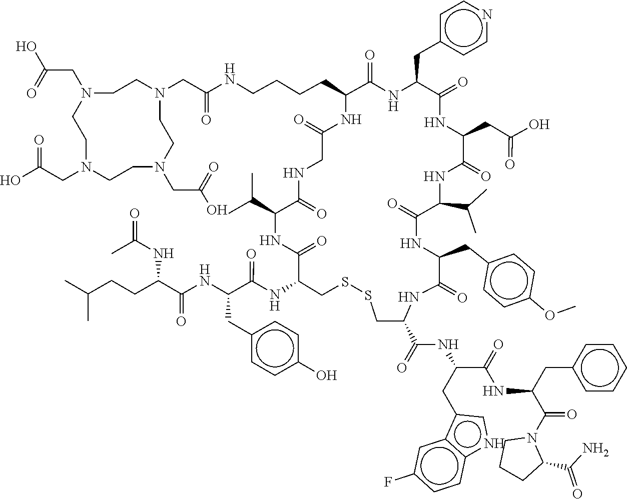 |
| 250 | 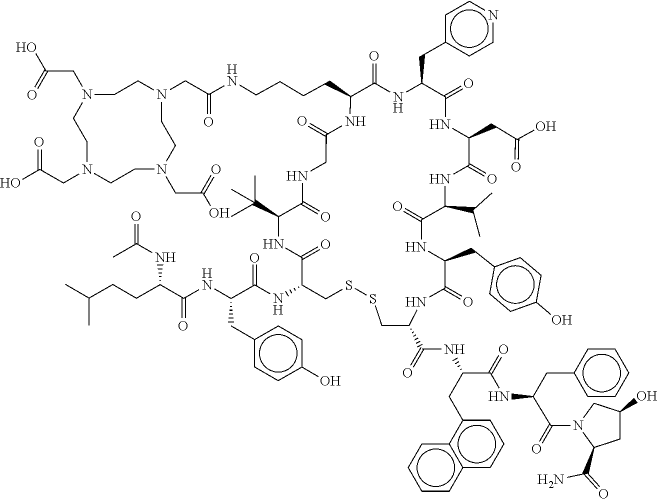 |
| 251 | 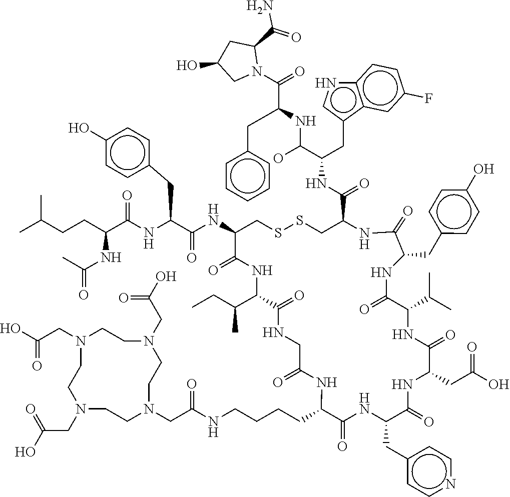 |
| 252 | 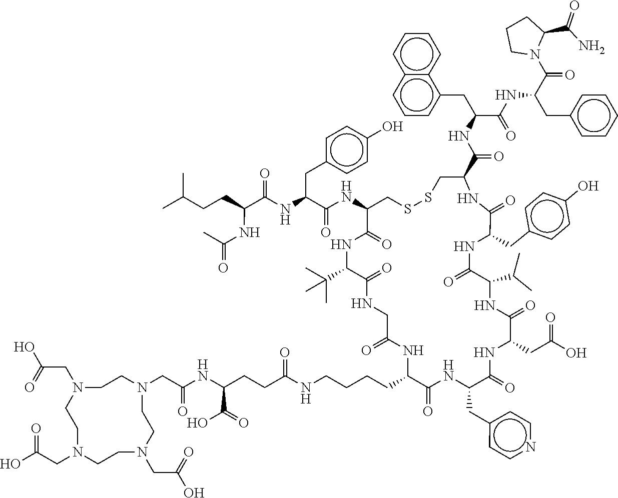 |
| 253 | 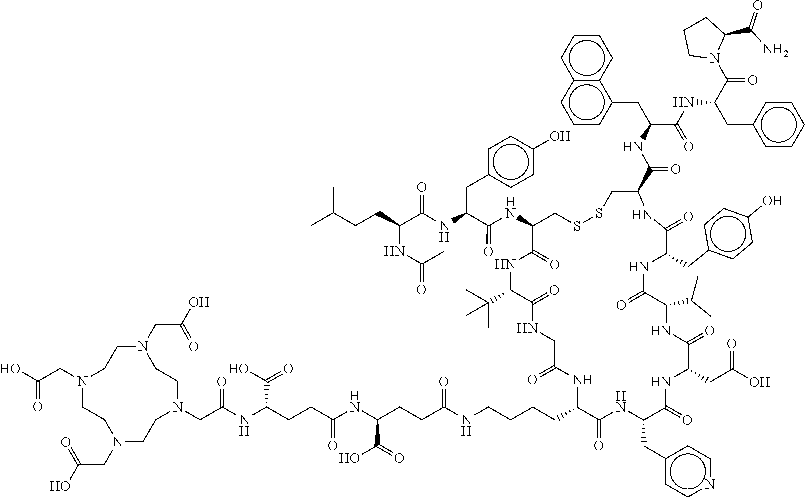 |
| 254 | 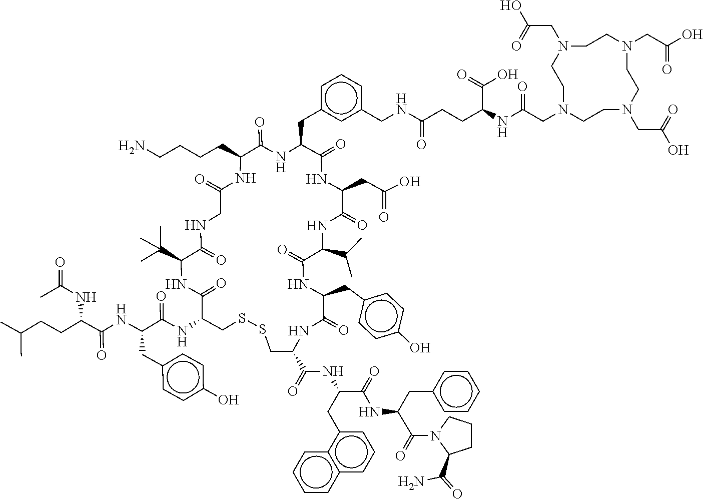 |
| 255 | 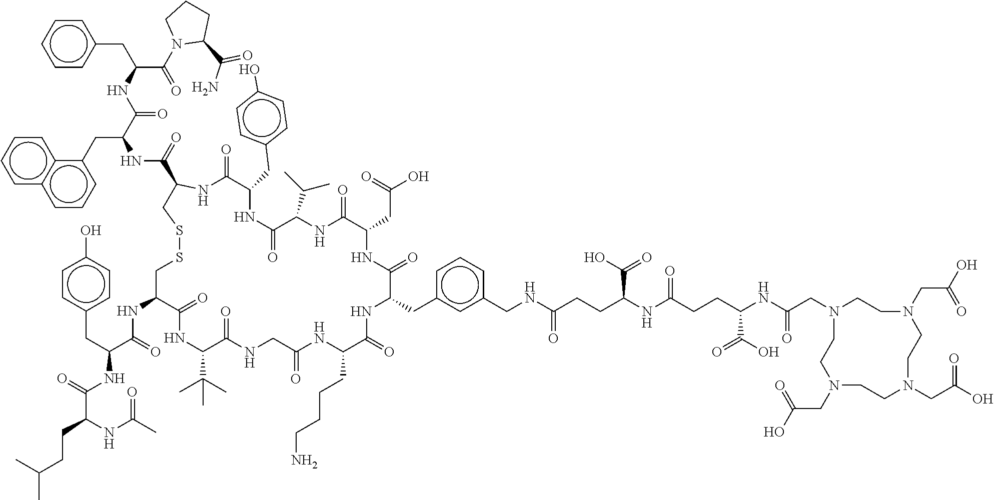 |
| 256 | 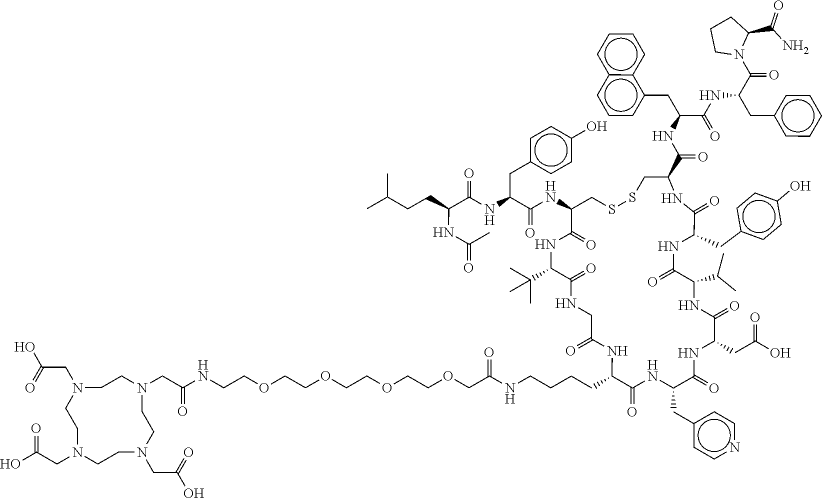 |
| 257 | 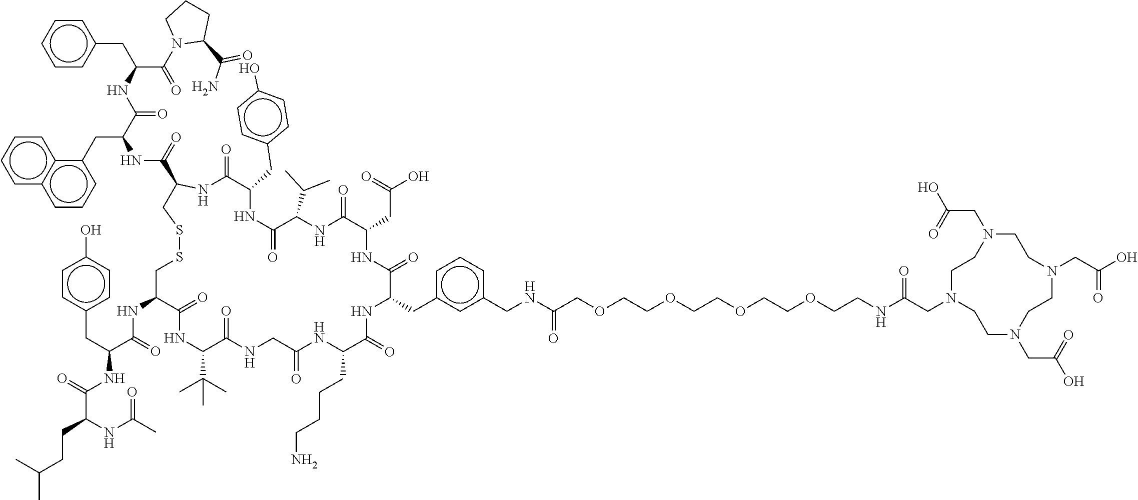 |
| 258 | 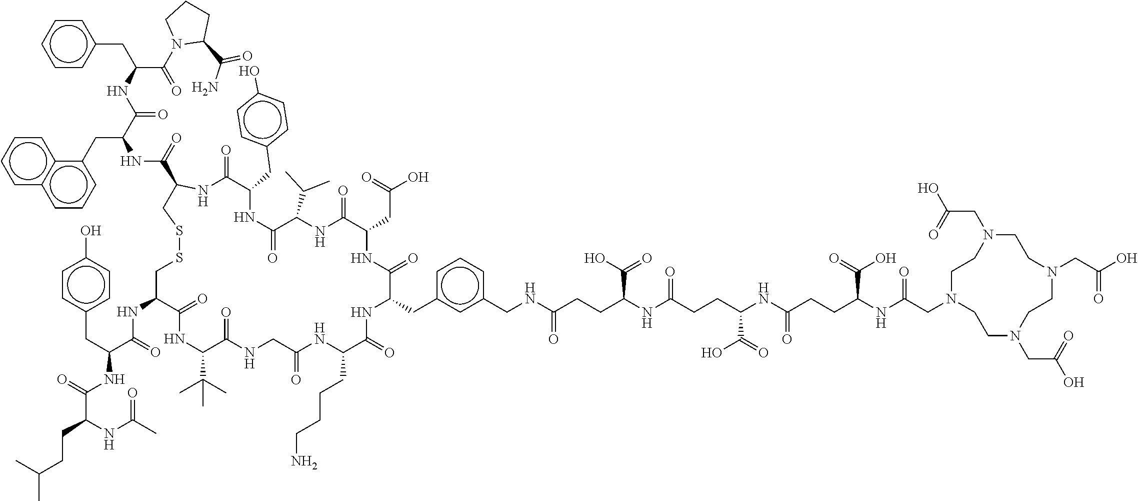 |
| 259 | 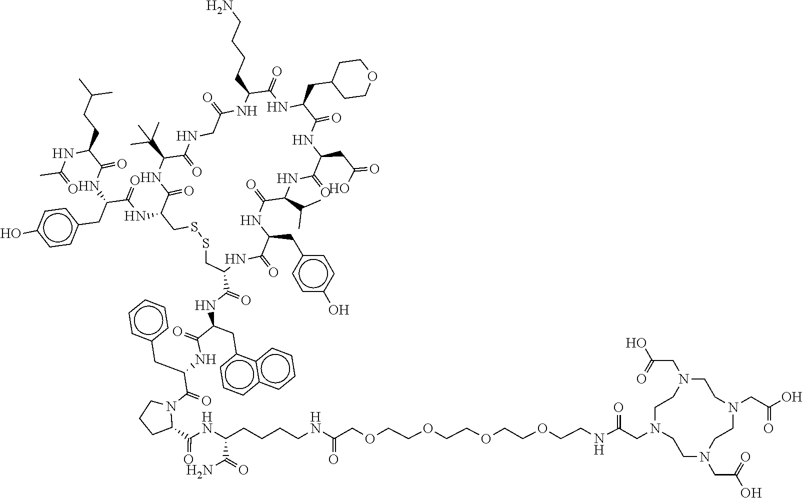 |
| 260 | 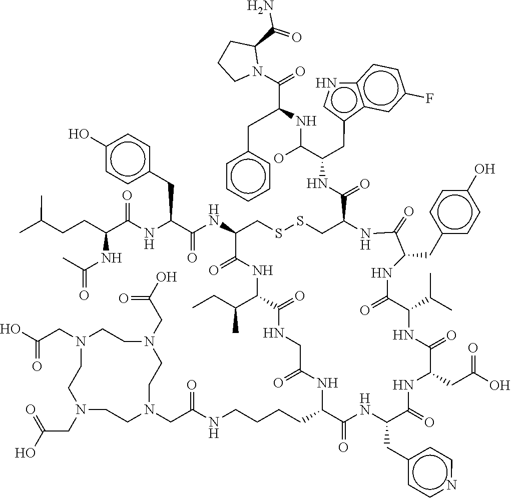 |
| 261 | 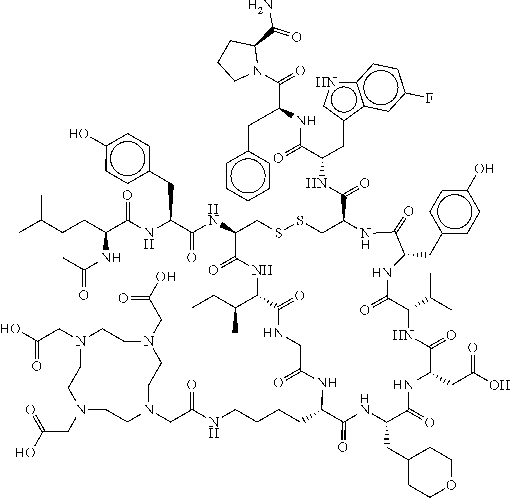 |
| 262 | 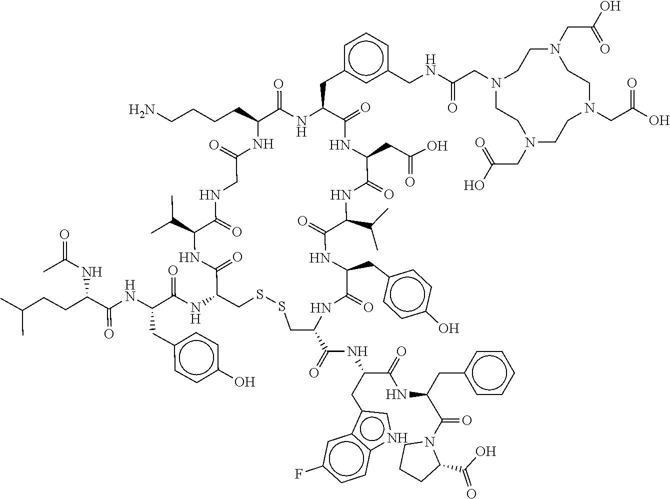 |
| 263 | 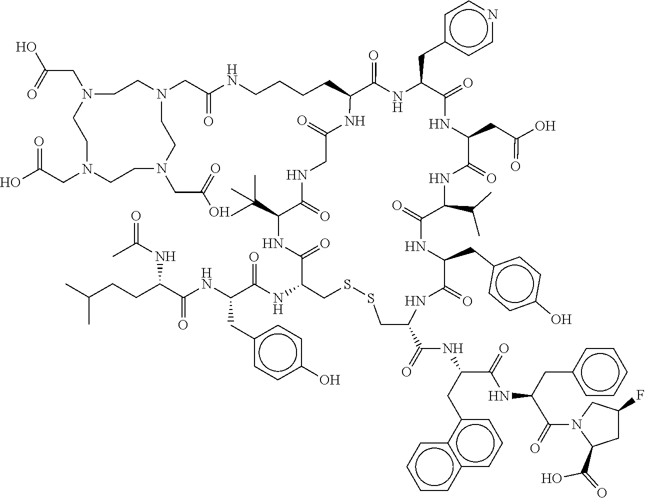 |
| 264 | 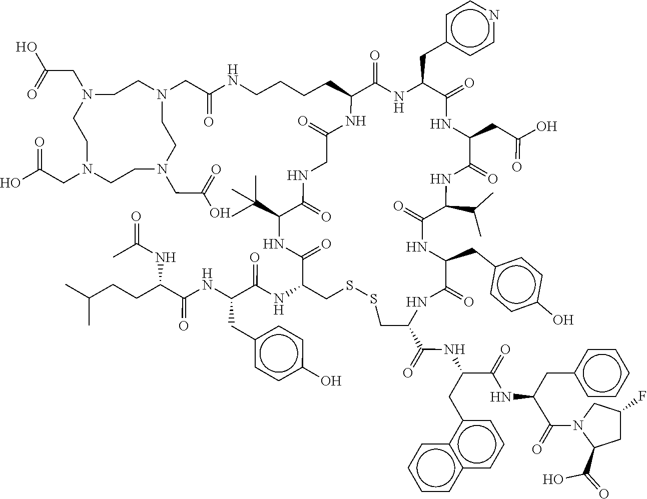 |
| 265 | 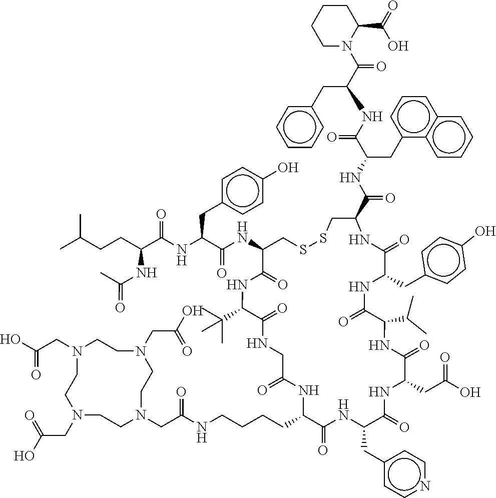 |
| 266 | 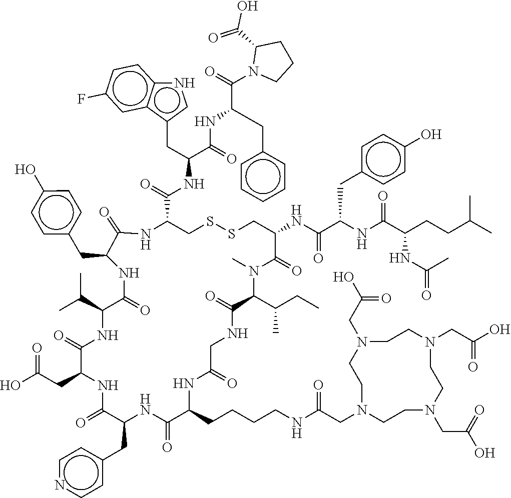 |
| 267 | 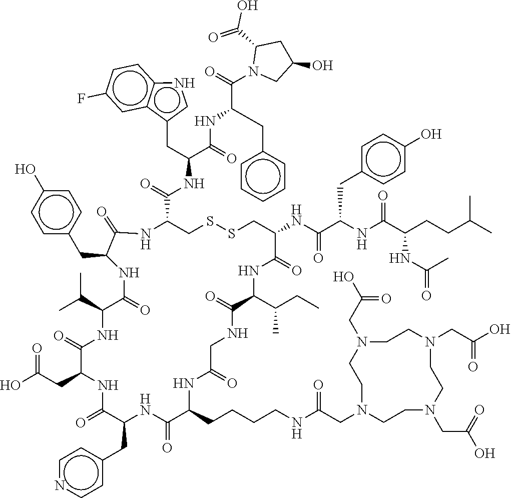 |
| 268 | 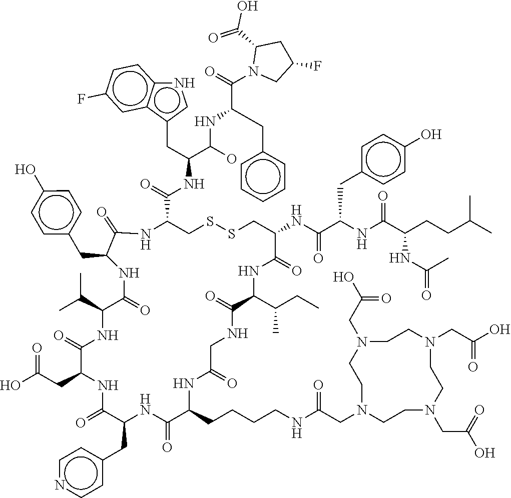 |
| 269 | 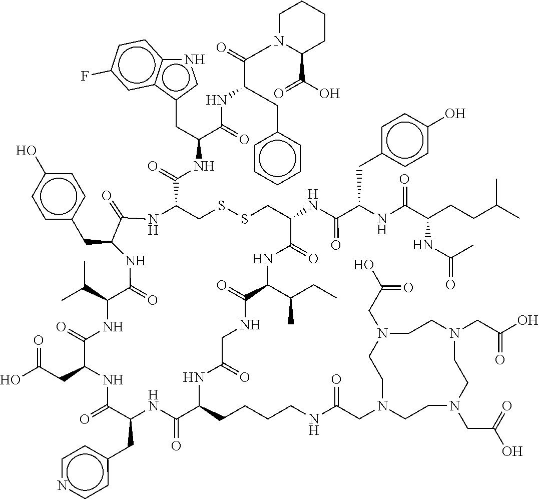 |
| 271 | 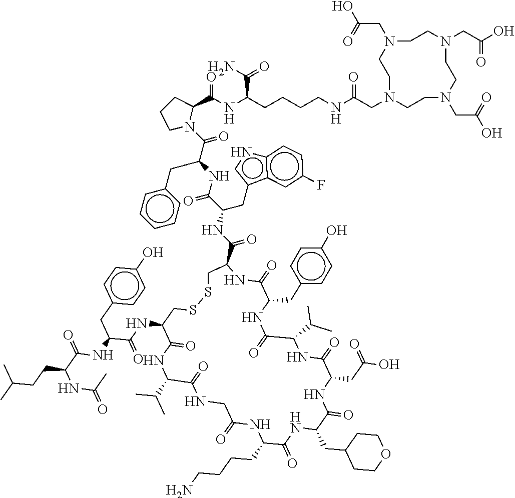 |
| 272 | 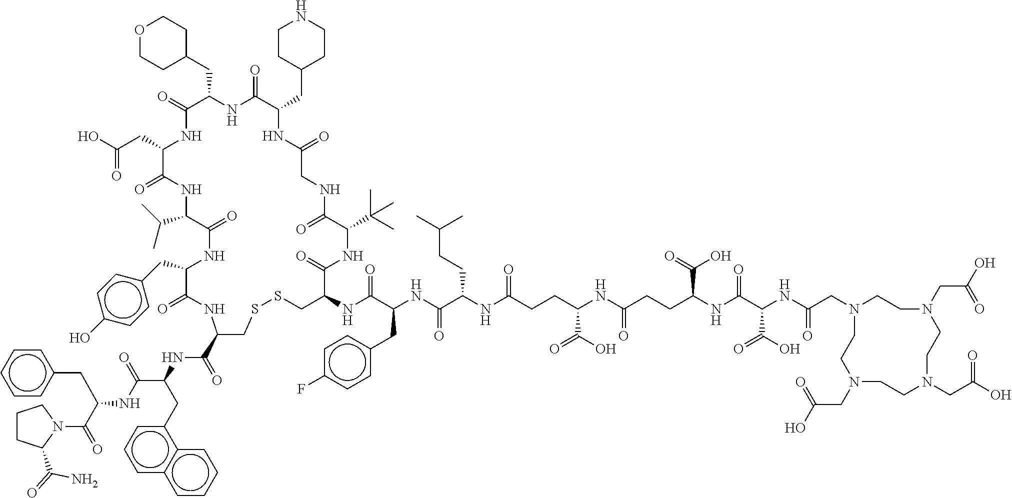 |
| 273 | 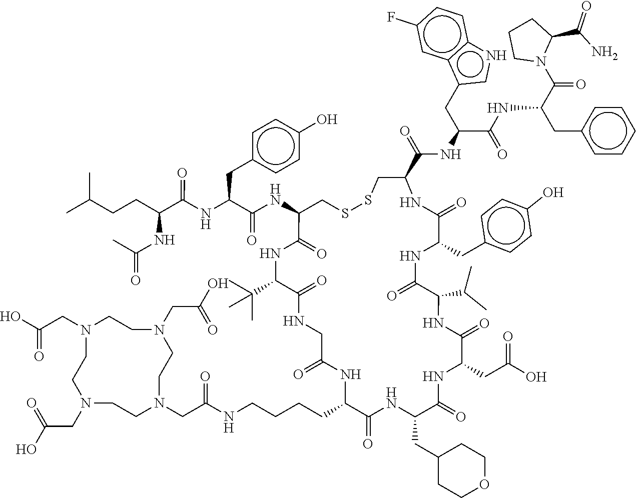 |
| 274 | 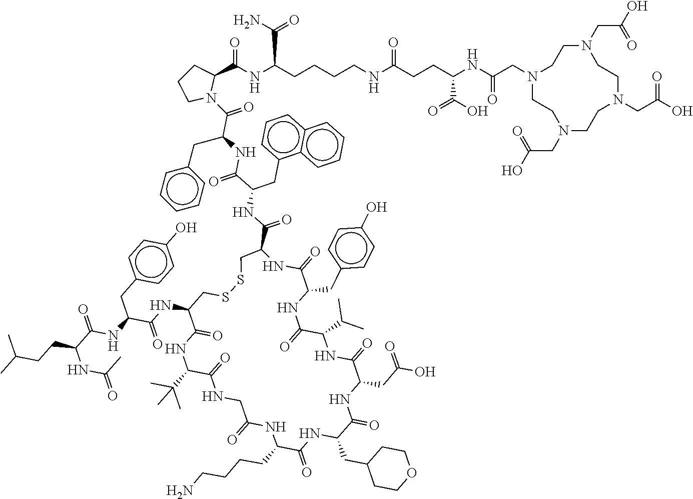 |
| 275 | 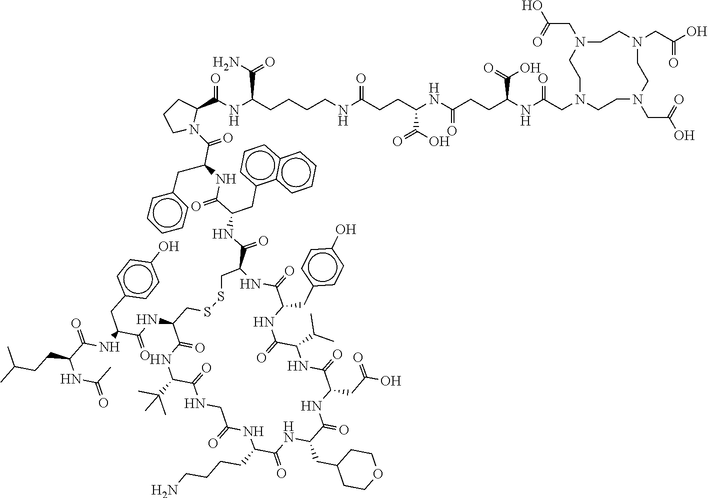 |
| 276 | 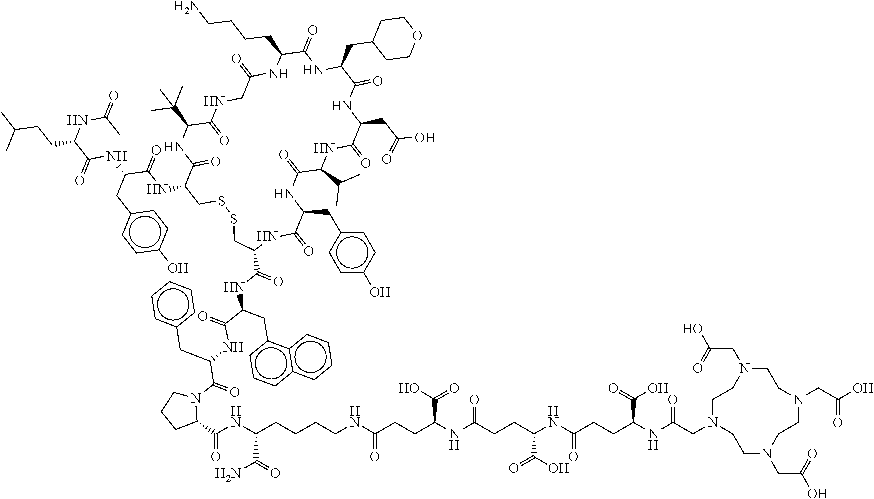 |
| 277 | 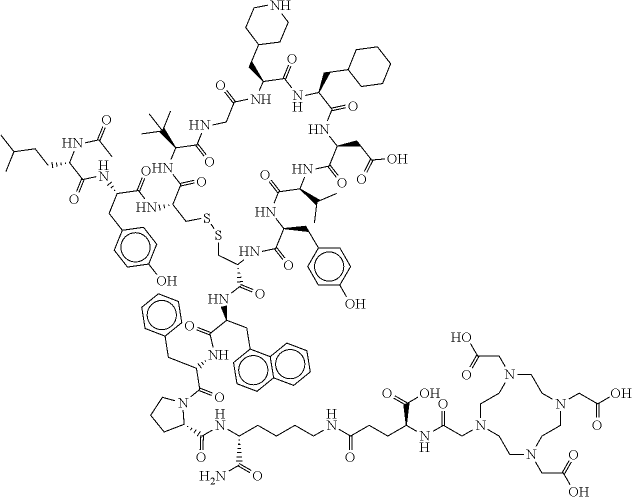 |
| 278 | 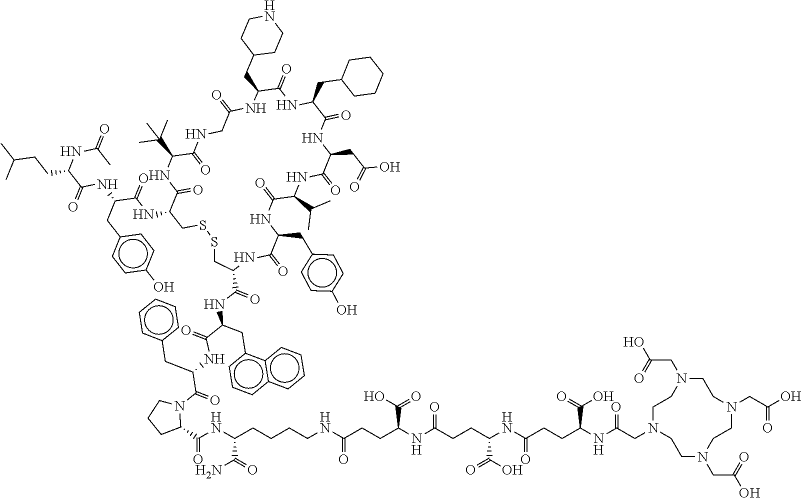 |
| 279 | 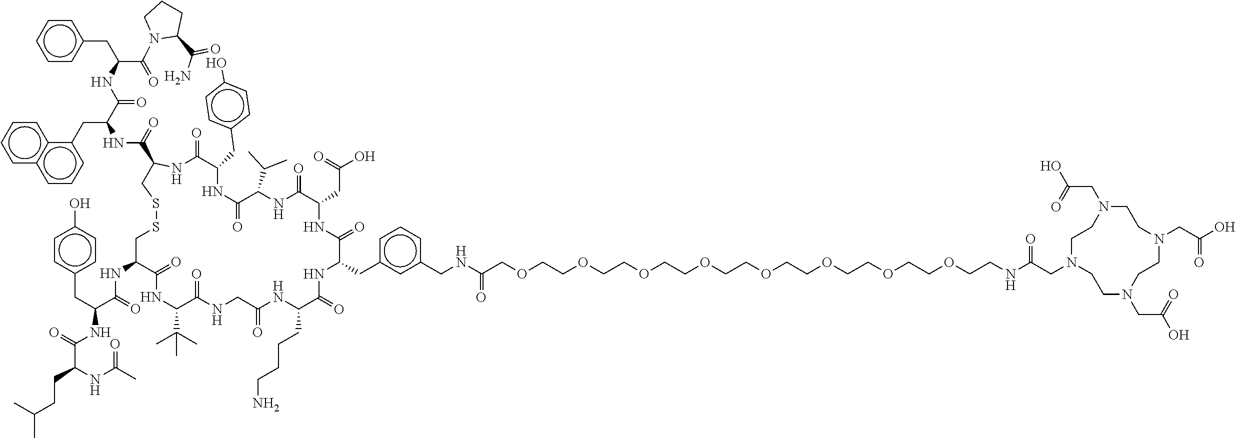 |
| 280 | 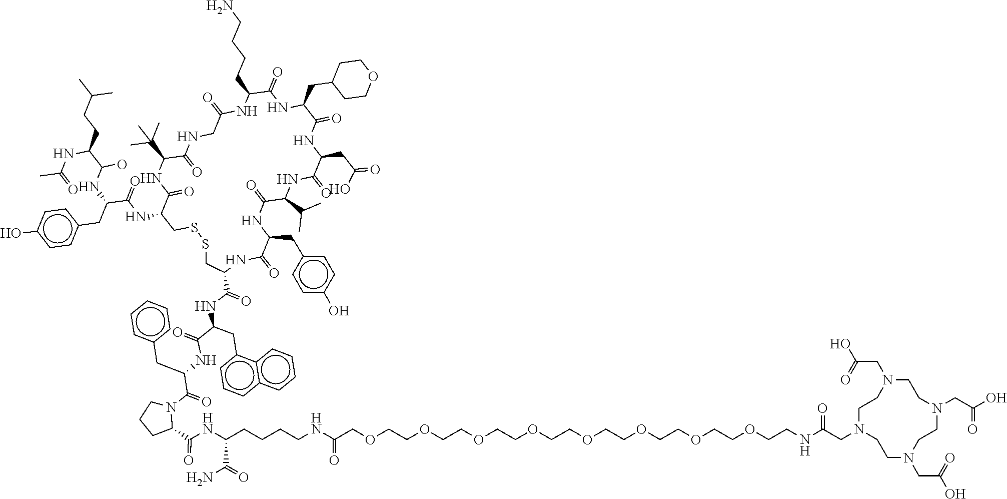 |
| 281 | 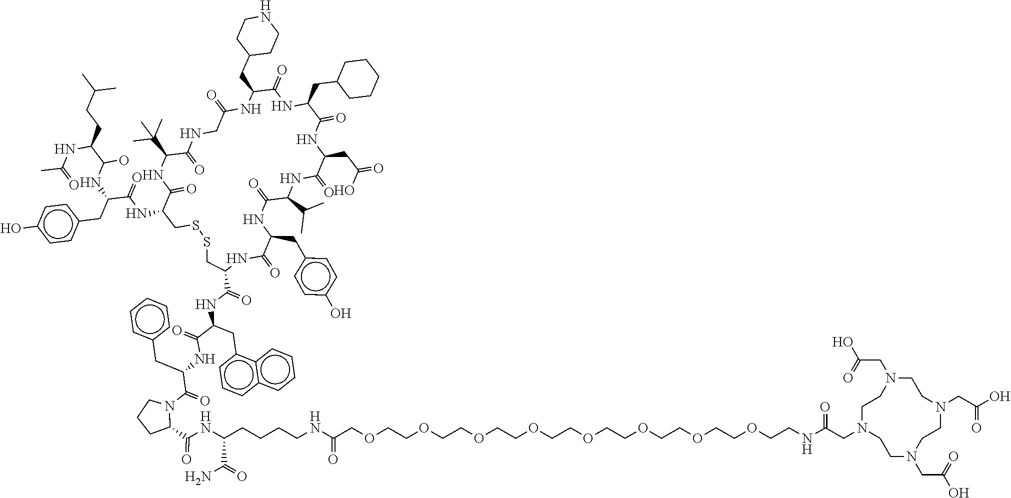 |
| 282 | 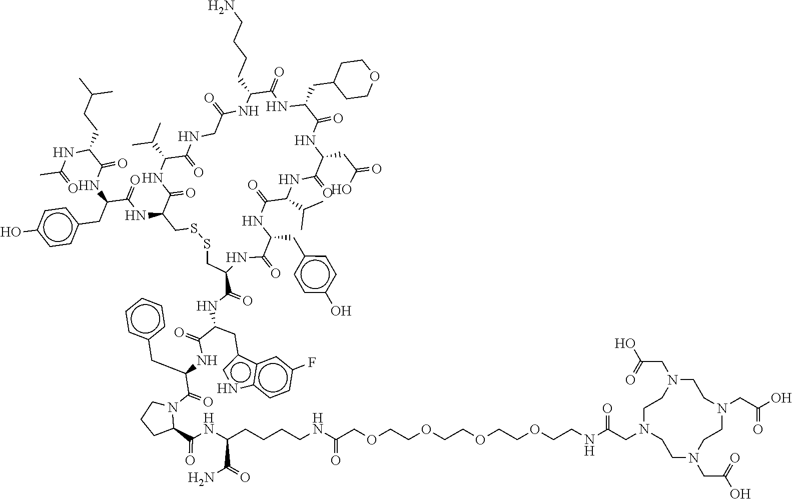 |
| 283 | 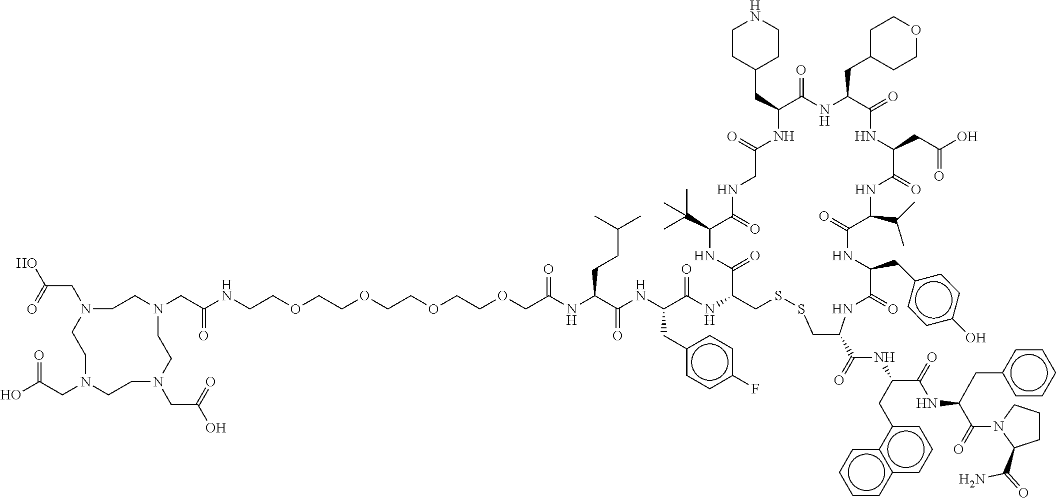 |
| 284 | 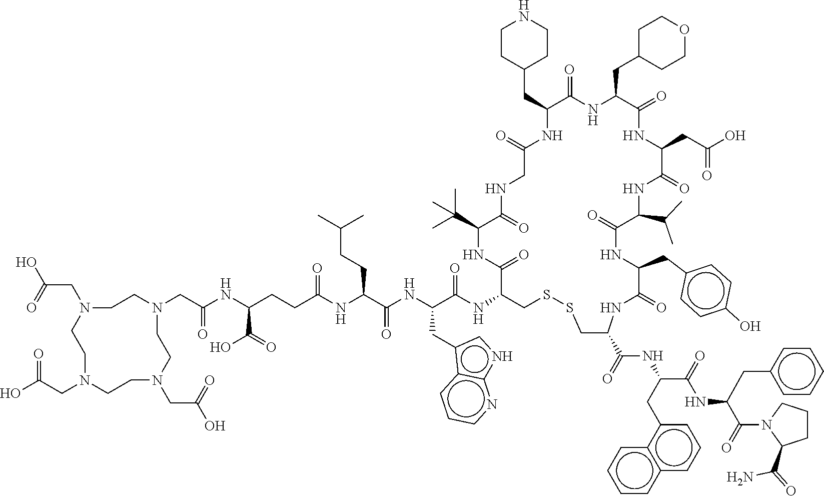 |
| 285 | 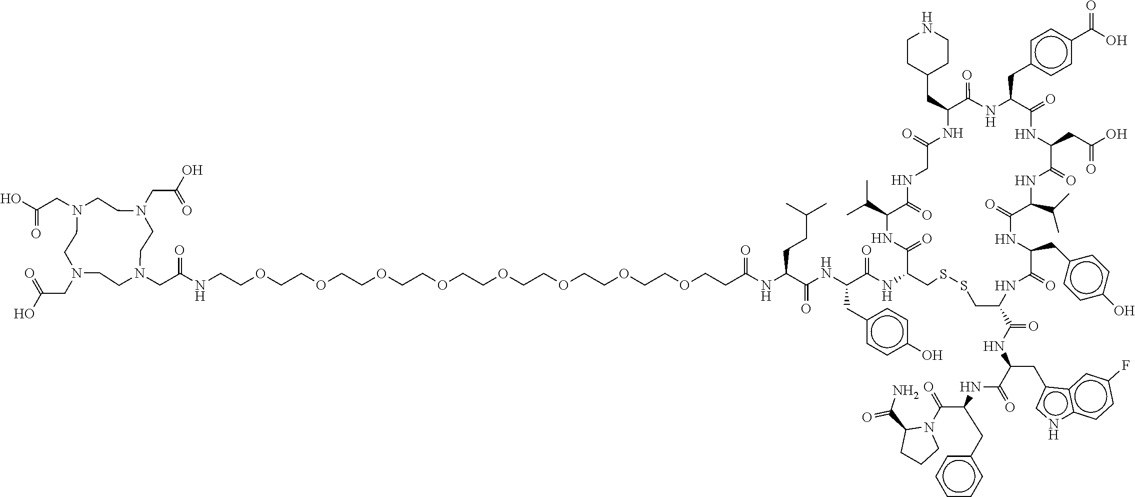 |
| 286 | 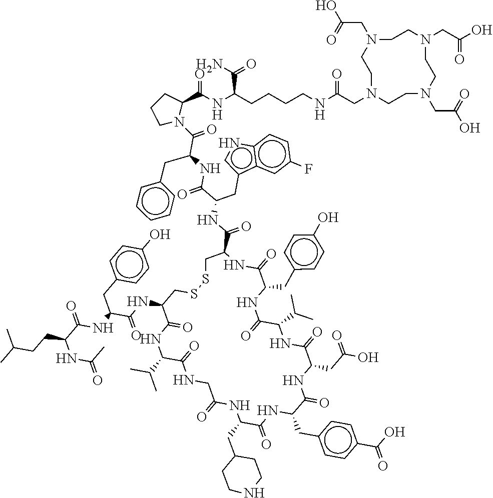 |
| 287 | 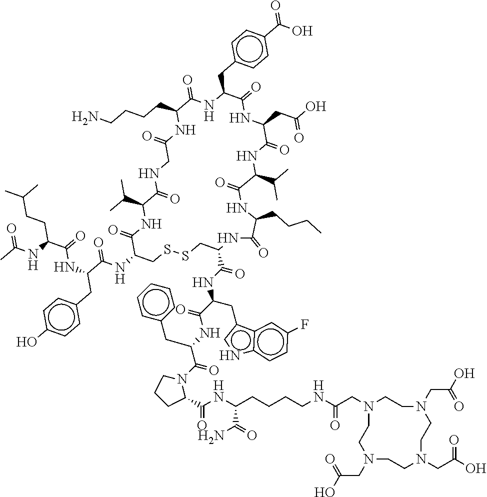 |
| 288 | 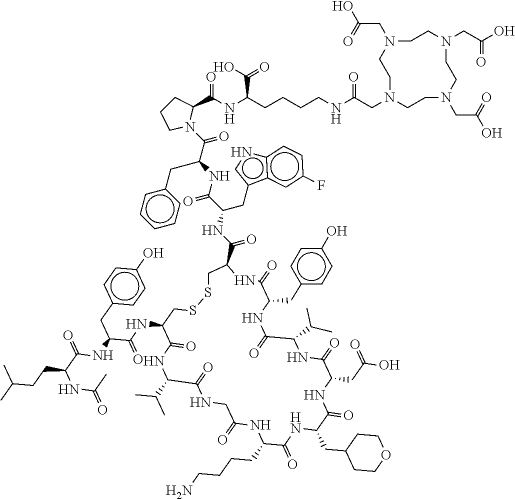 |
| 289 | 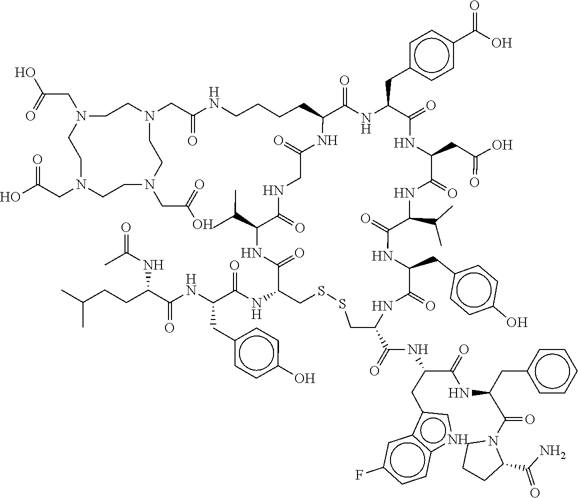 |
| 290 | 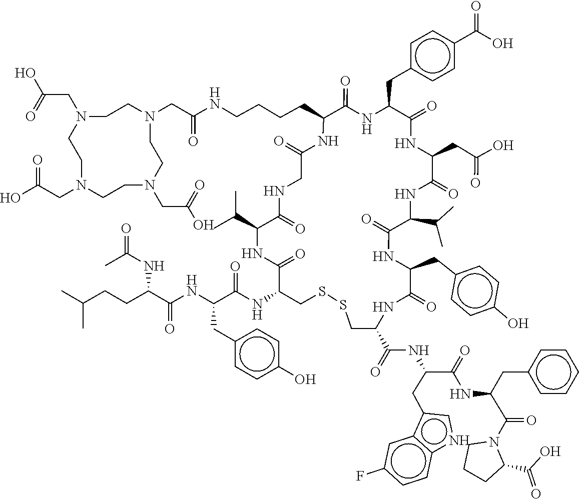 |
| 291 | 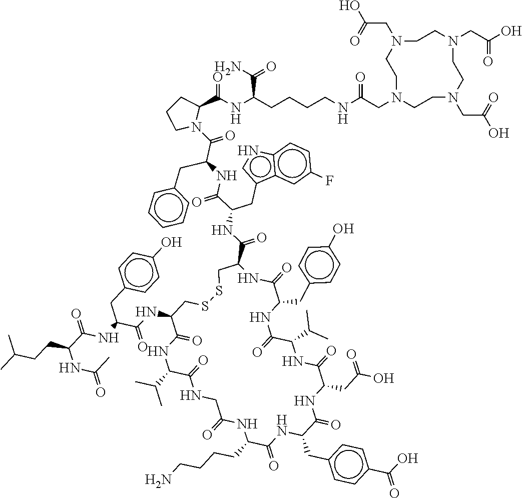 |
| 292 | 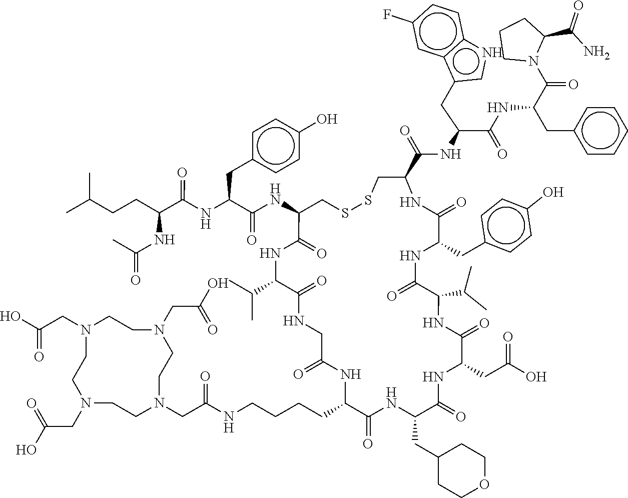 |
| 293 | 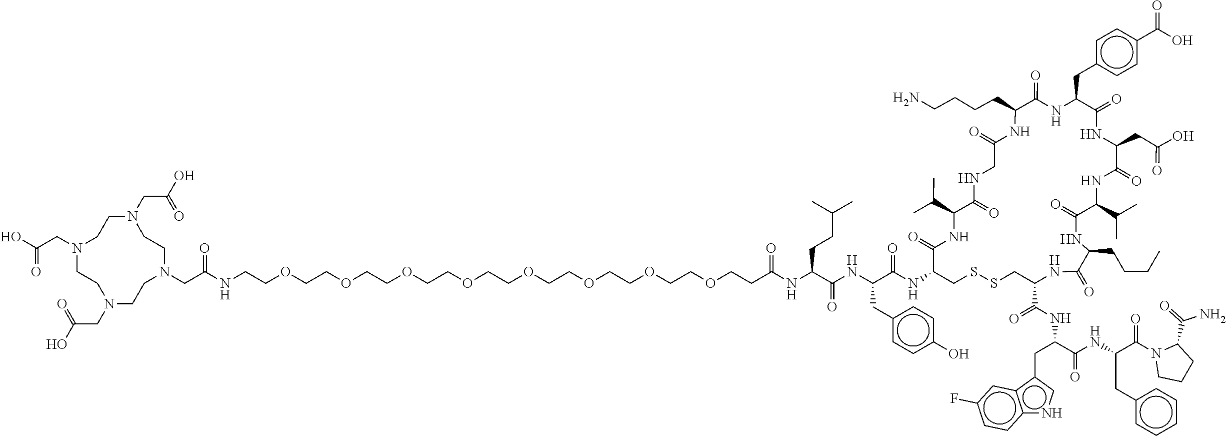 |
| 294 | 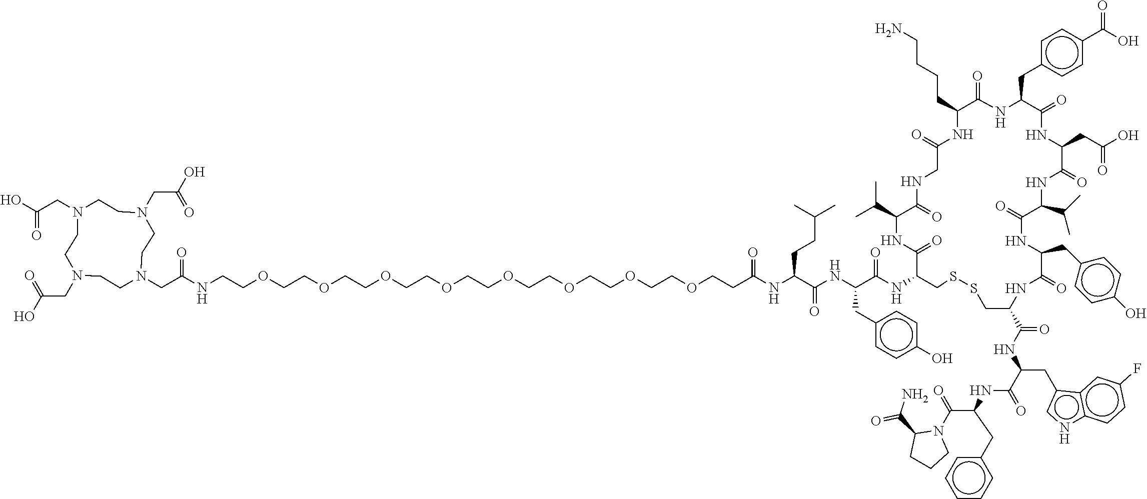 |
| 295 | 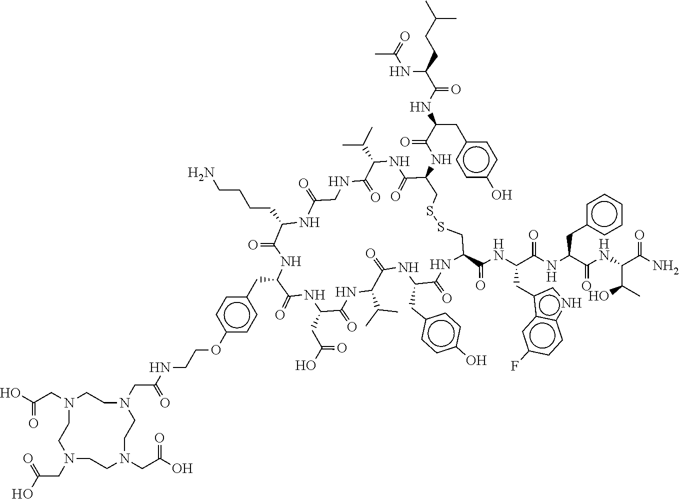 |
| 297 | 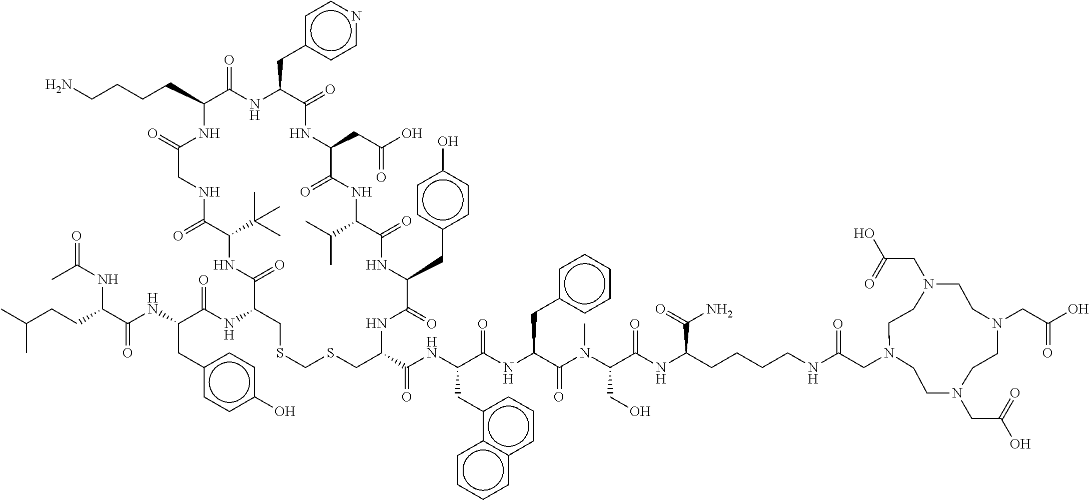 |
| 298 | 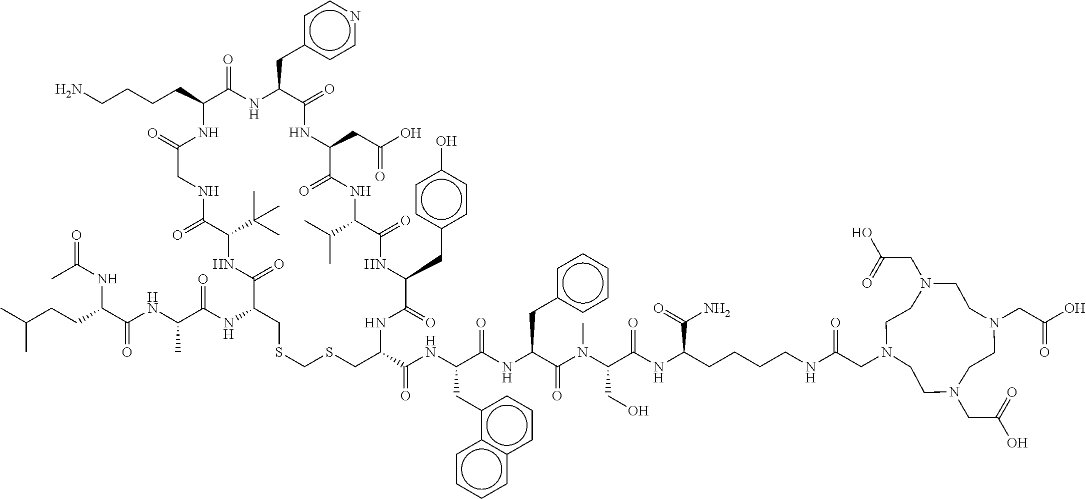 |
| 299 | 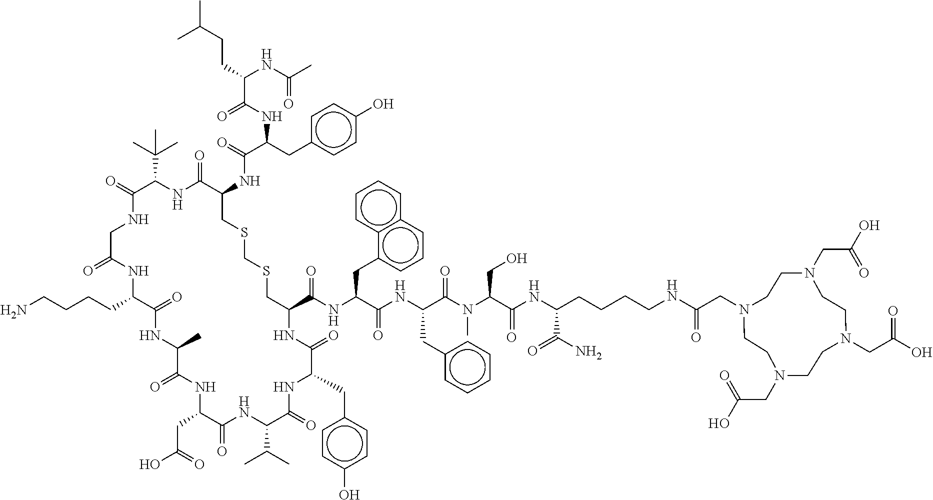 |
| 300 | 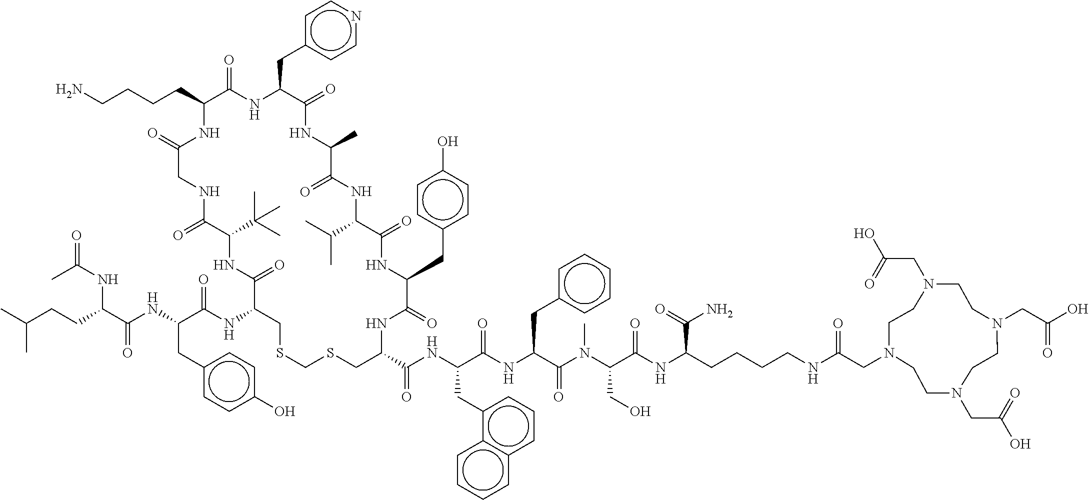 |
| 301 | 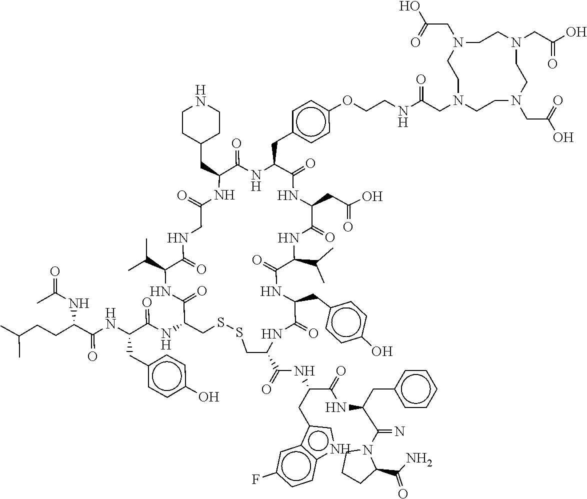 |
| 302 | 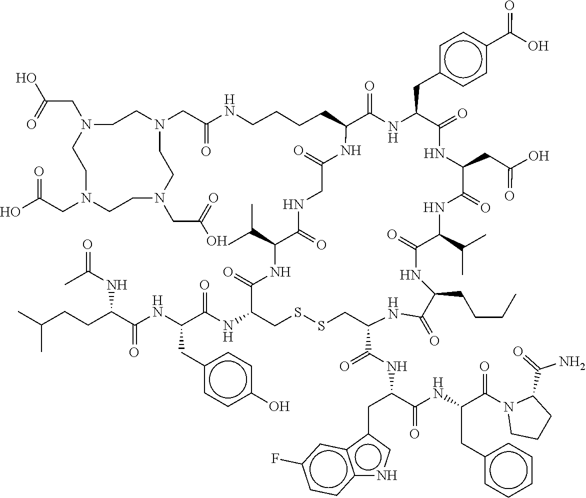 |
| 303 | 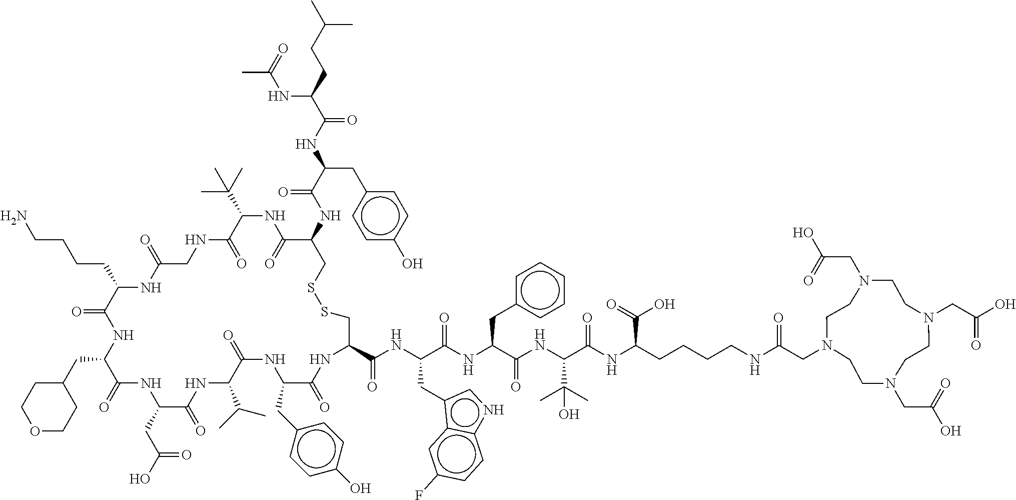 |
| 304 | 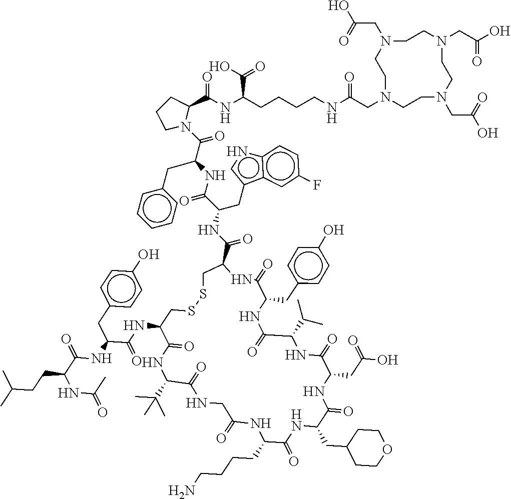 |
| 305 | 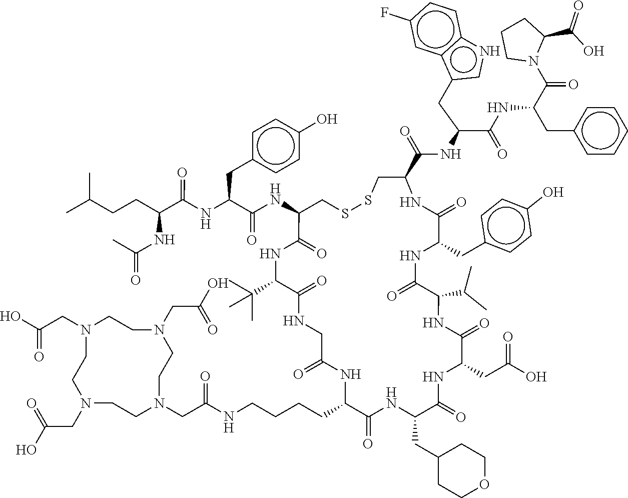 |
| 306 | 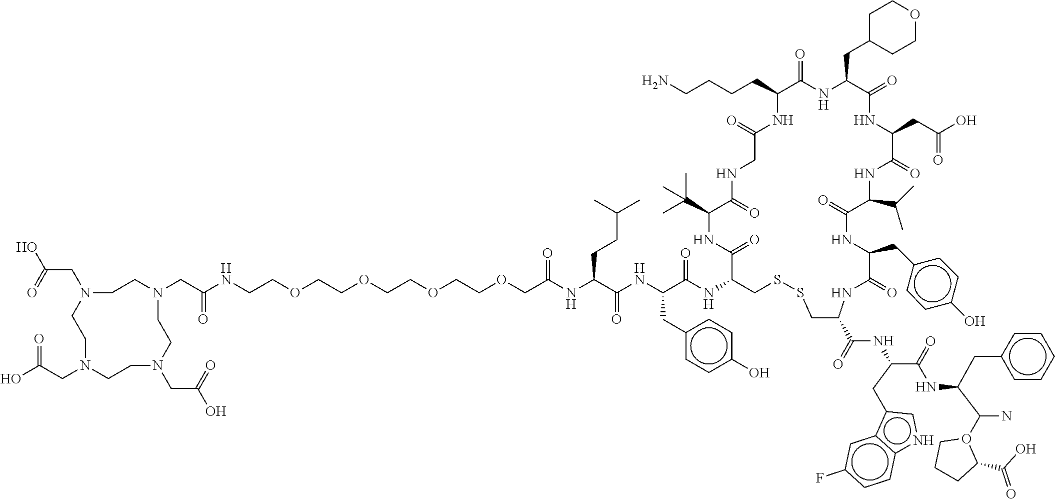 |
| 307 | 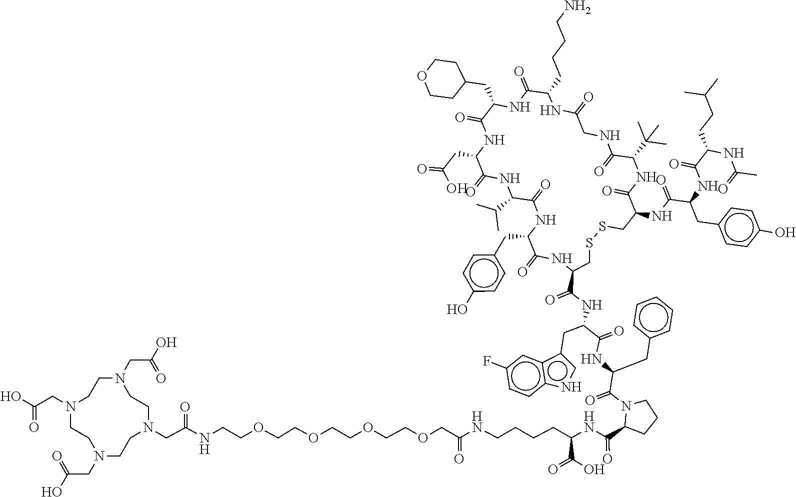 |
| 308 | 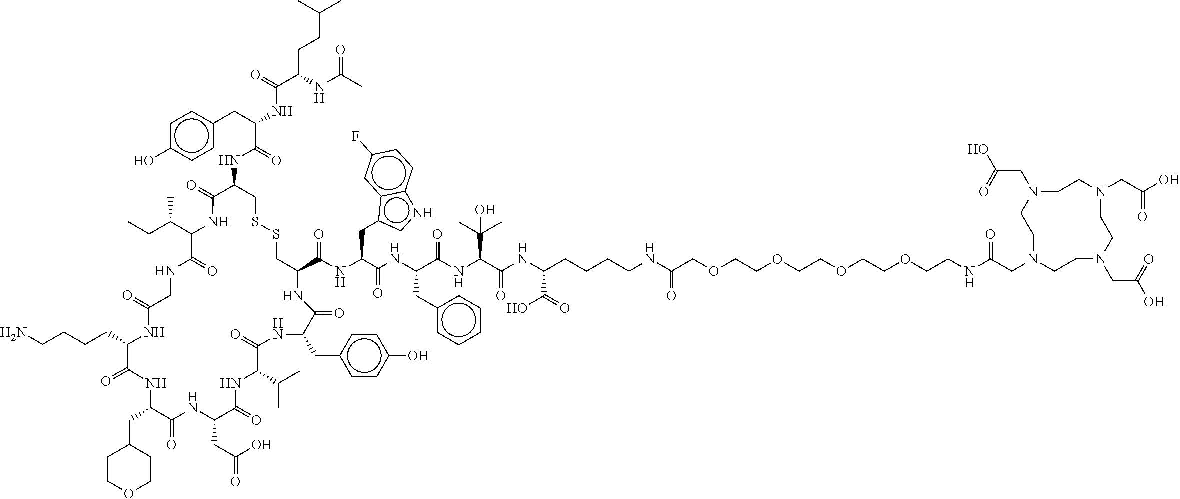 |
| 309 | 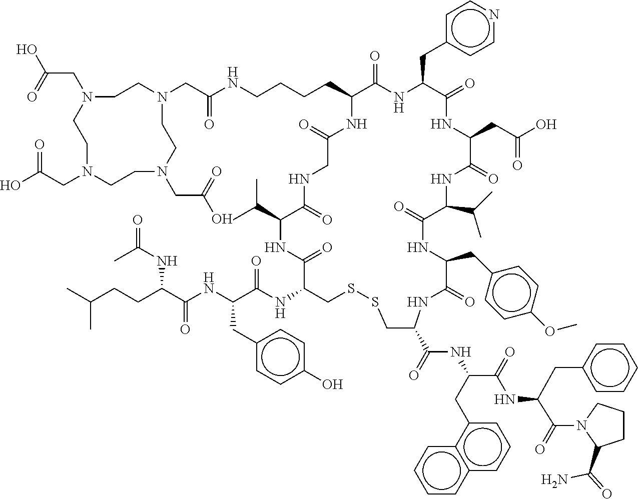 |
| 310 | 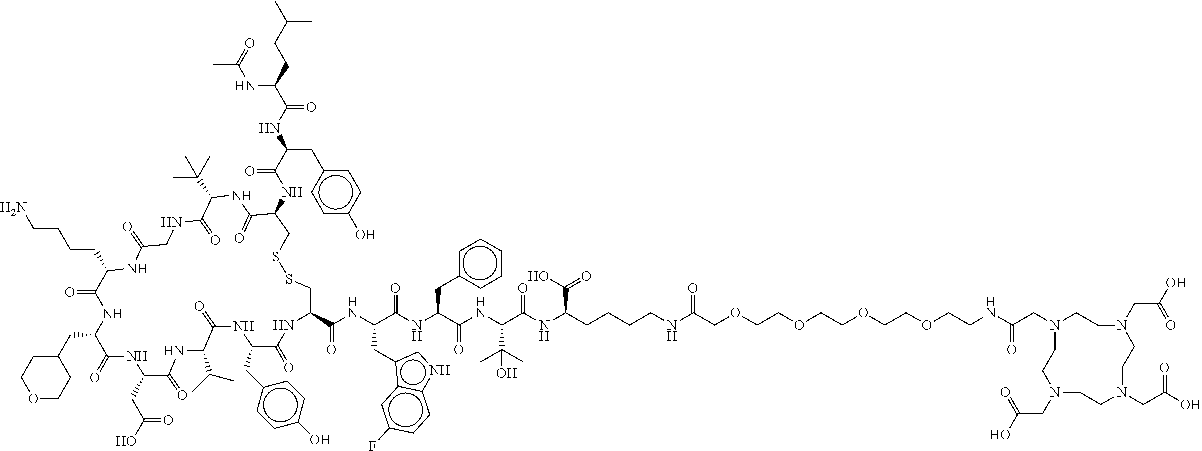 |
| 311 | 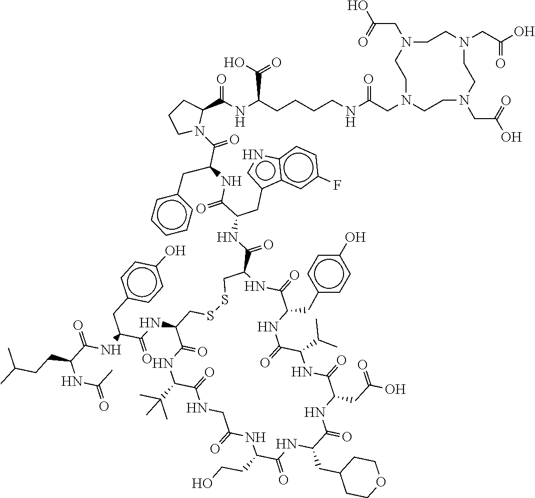 |
| 312 | 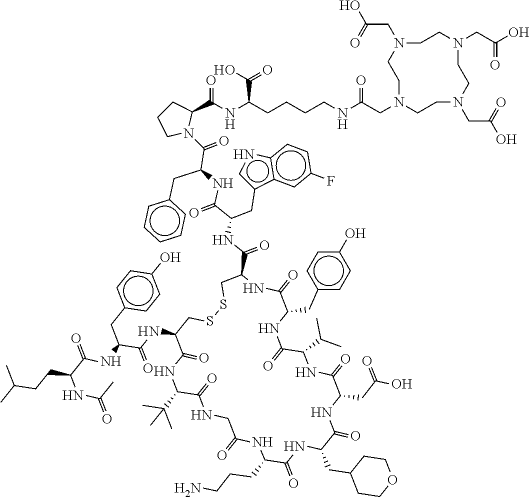 |
| 313 | 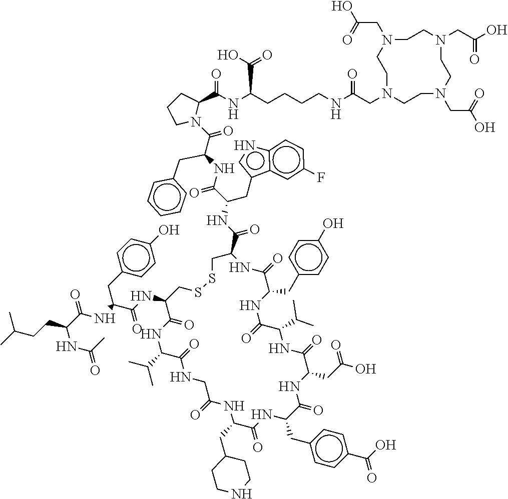 |
| 314 | 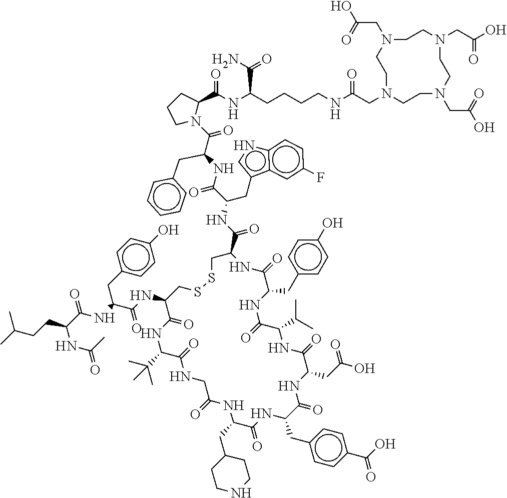 |
| 315 | 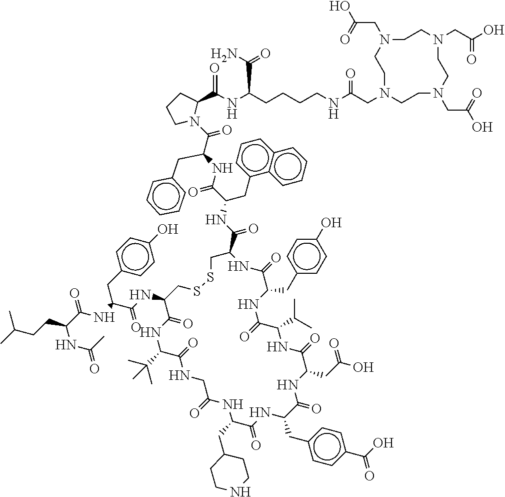 |
| 316 | 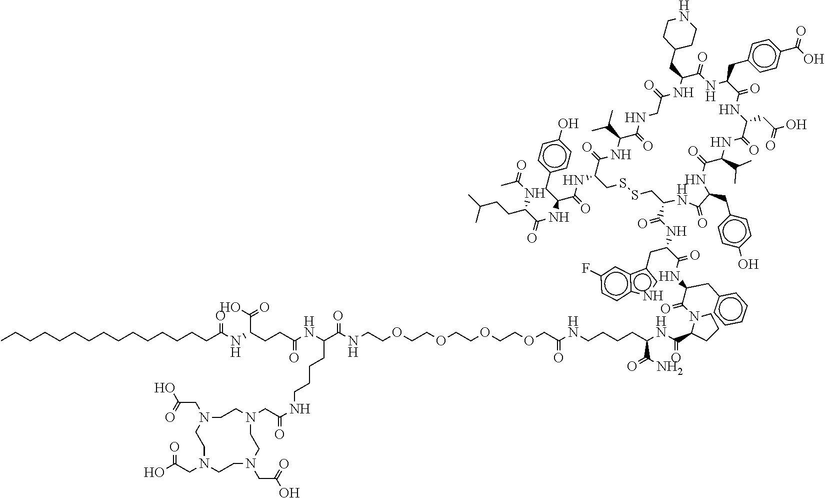 |
| 317 | 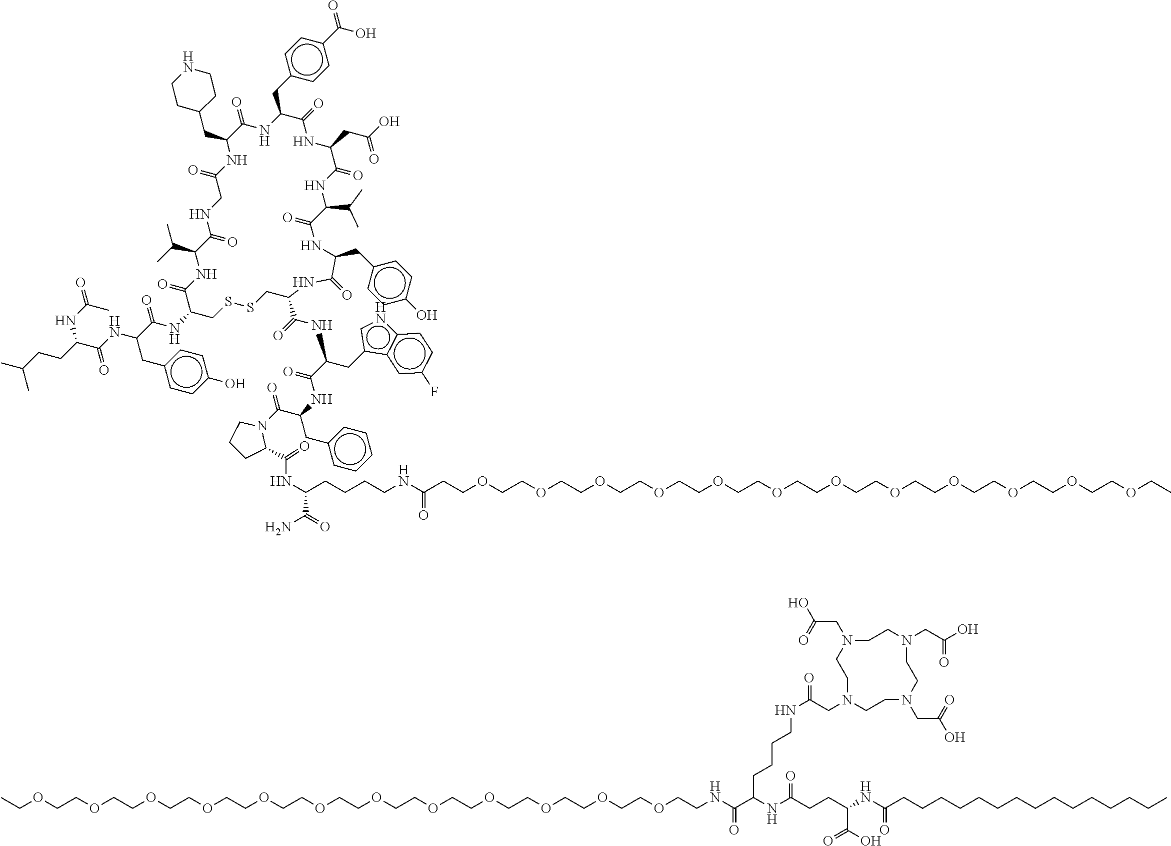 |
| 318 | 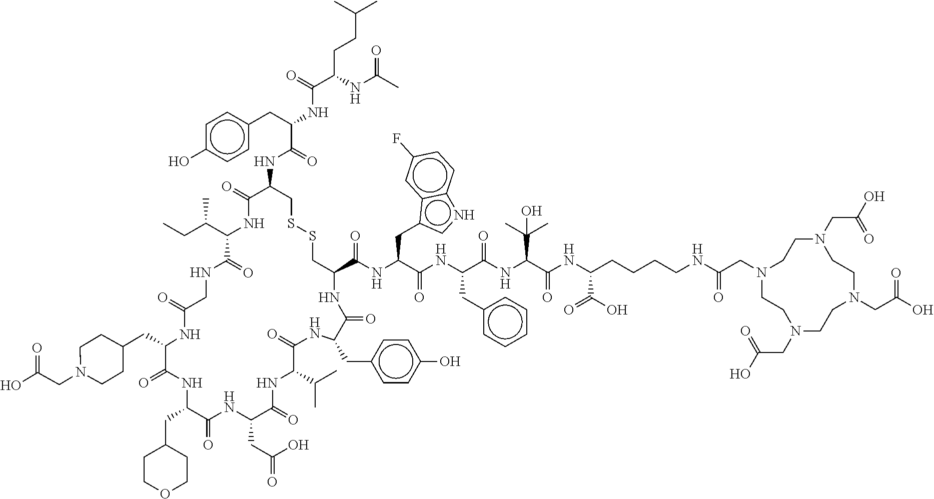 |
| 319 | 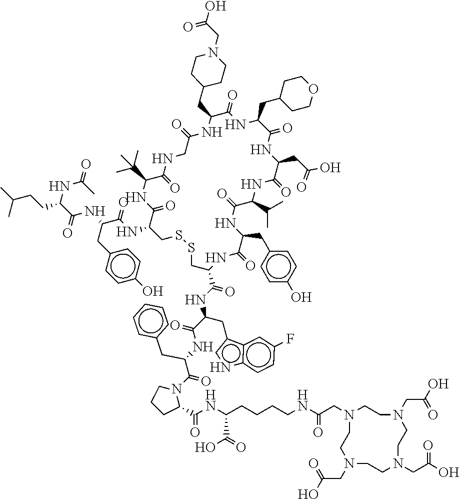 |
| 320 | 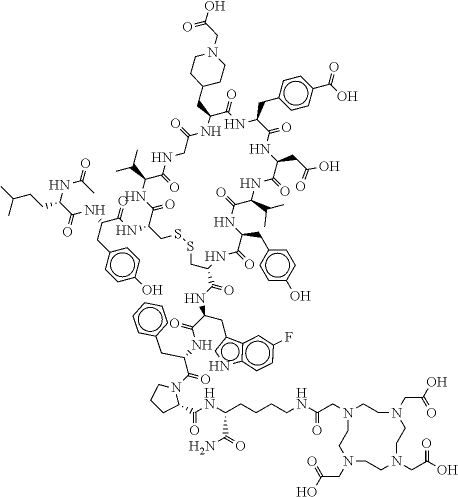 |
| 321 | 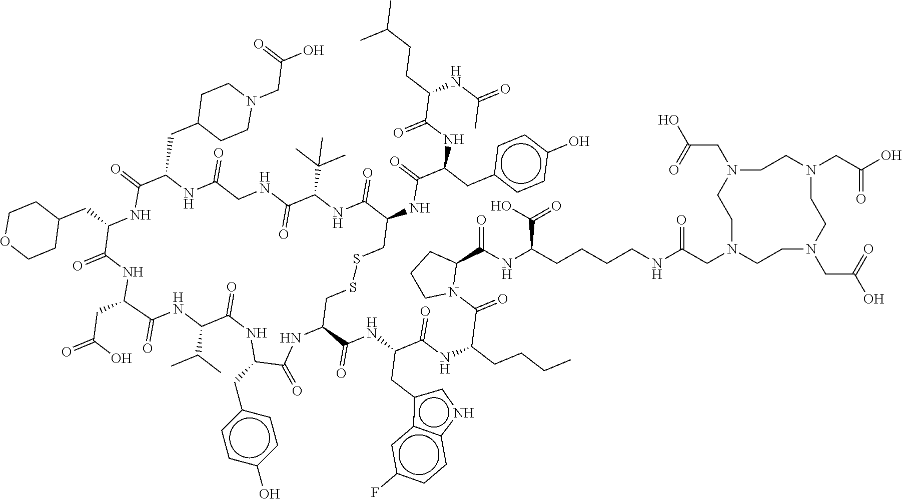 |
| 322 | 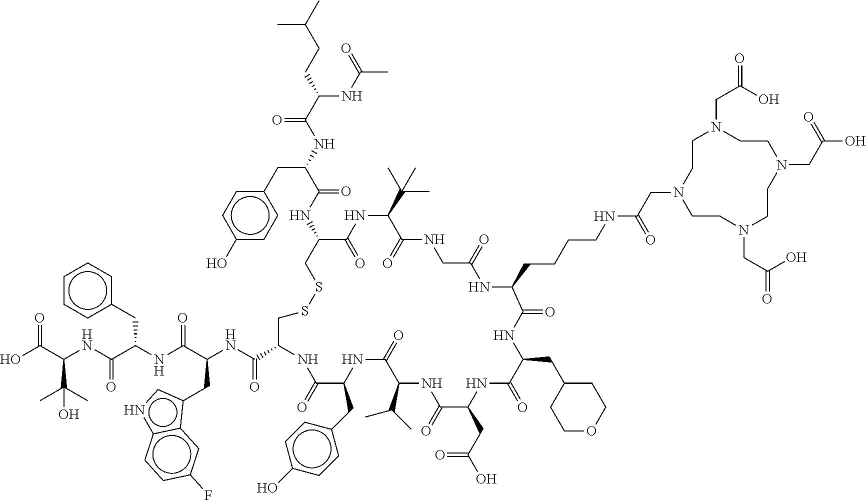 |
| 323 | 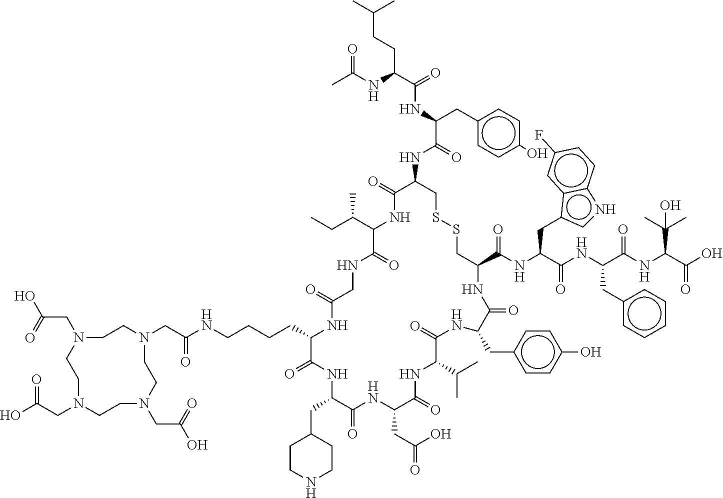 |
| 324 | 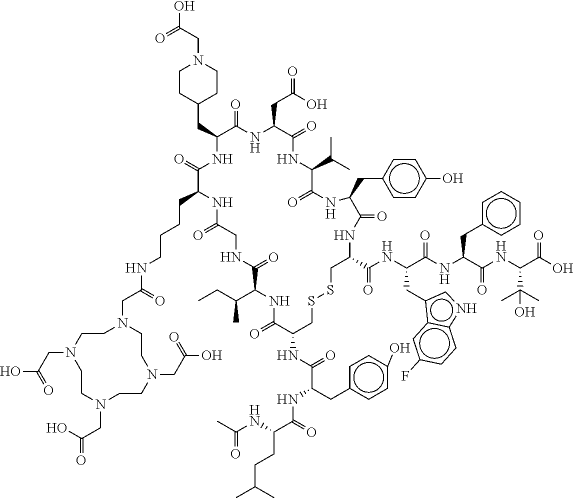 |
| 325 | 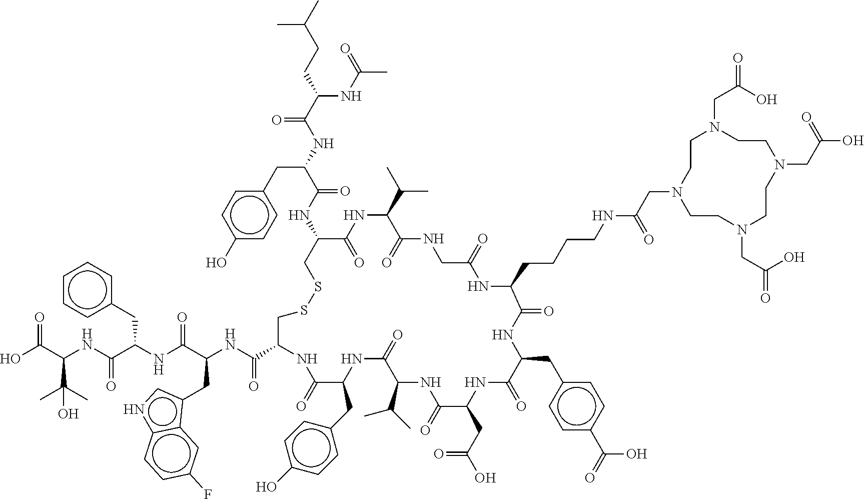 |
| 326 | 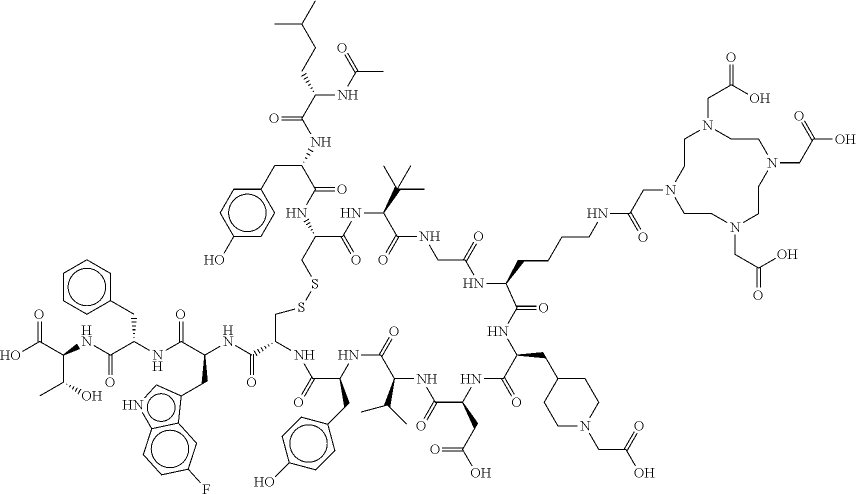 |
| 327 | 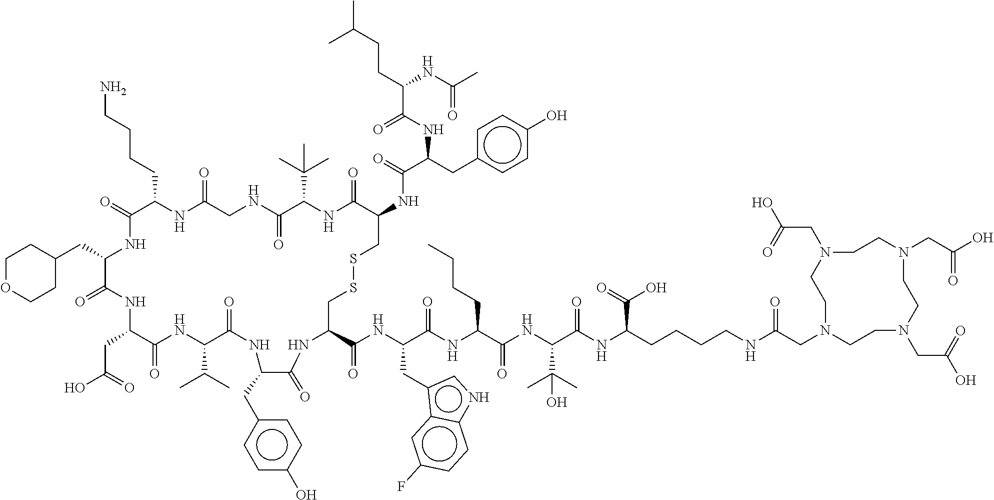 |
| 328 | 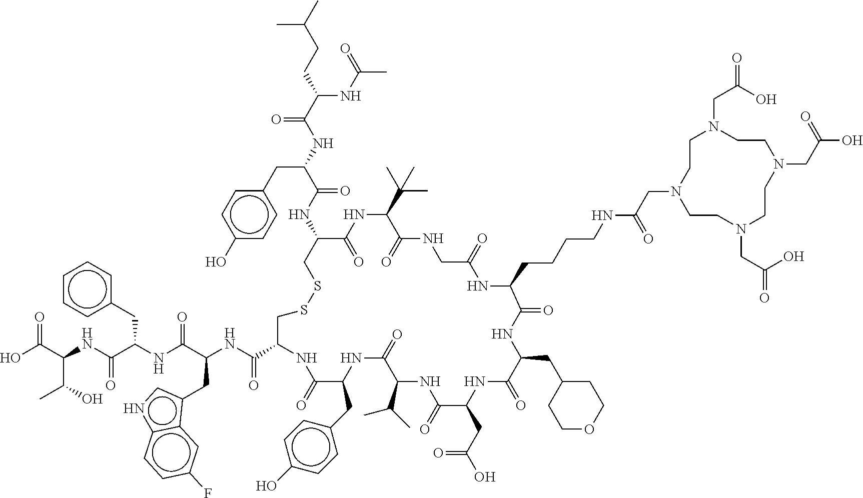 |
| 329 | 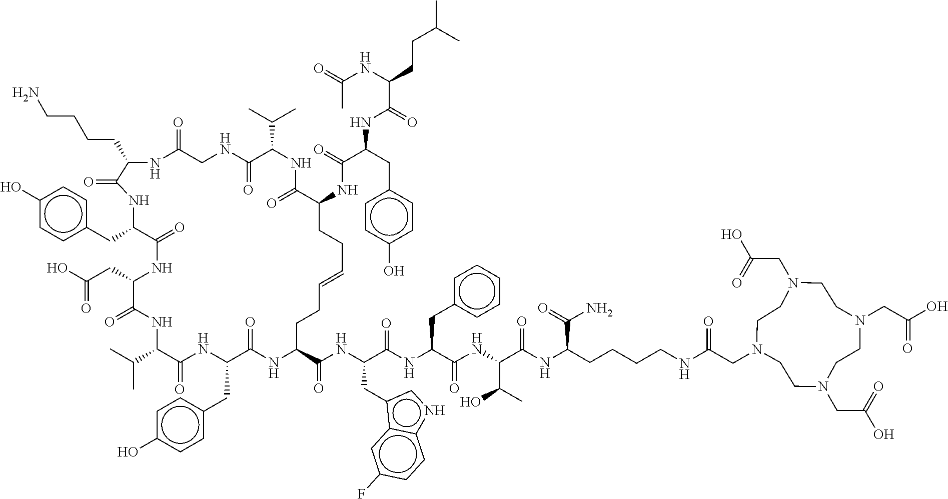 |
| 330 | 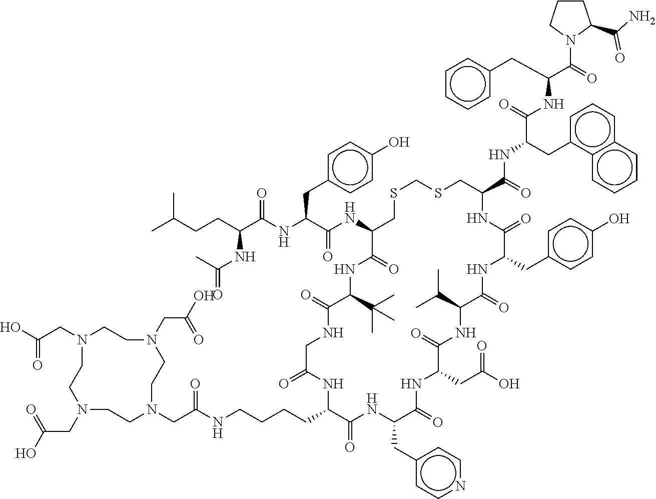 |
| 331 | 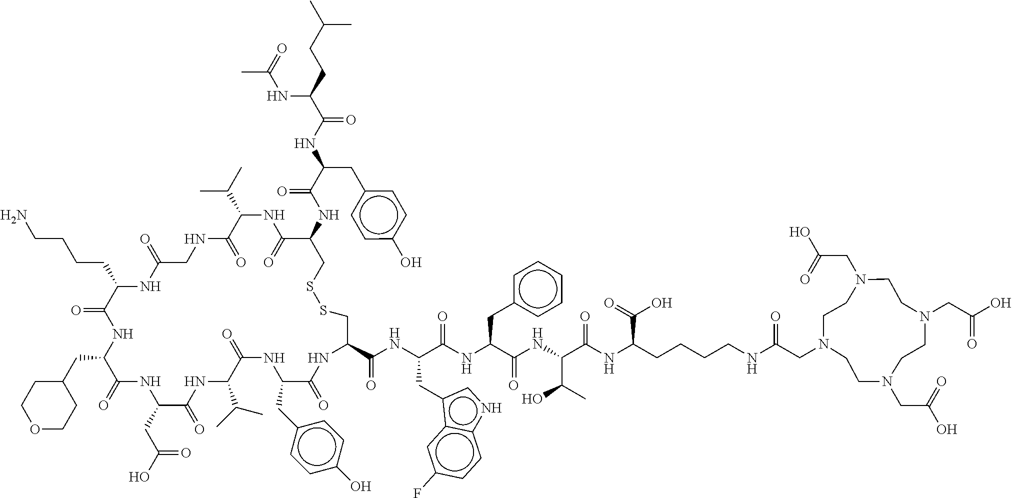 |
| 332 | 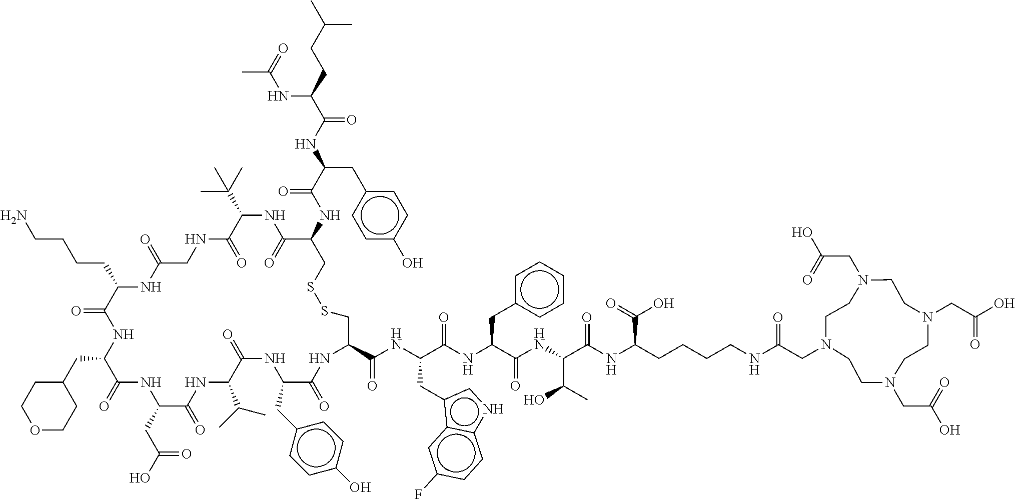 |
| 333 | 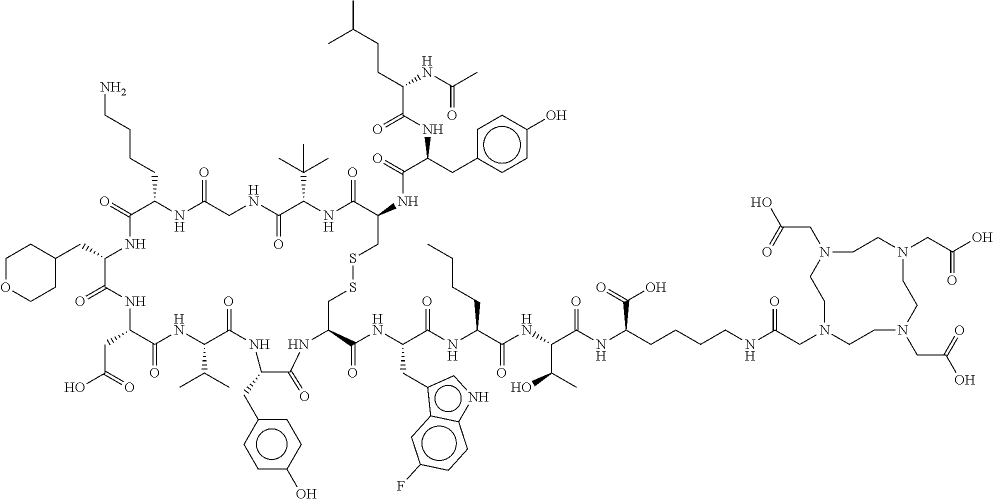 |
| 334 | 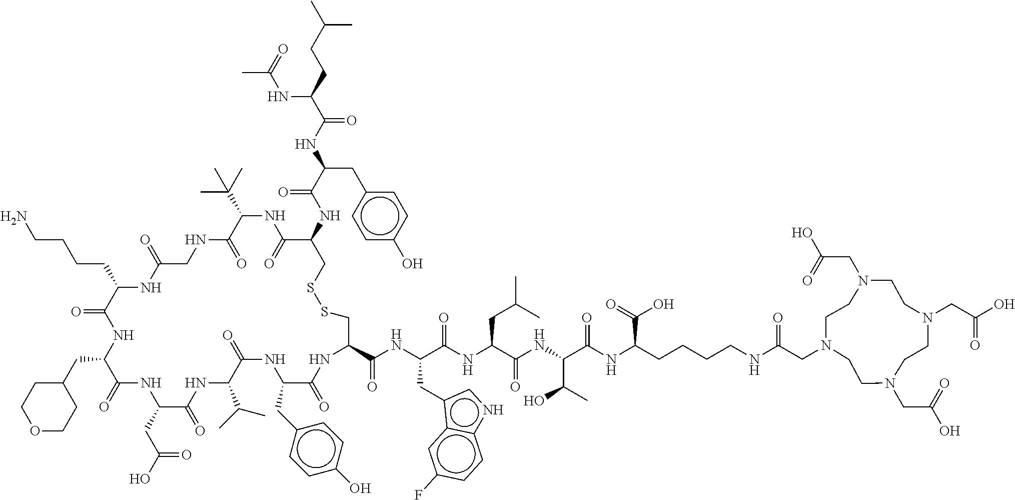 |
| 335 | 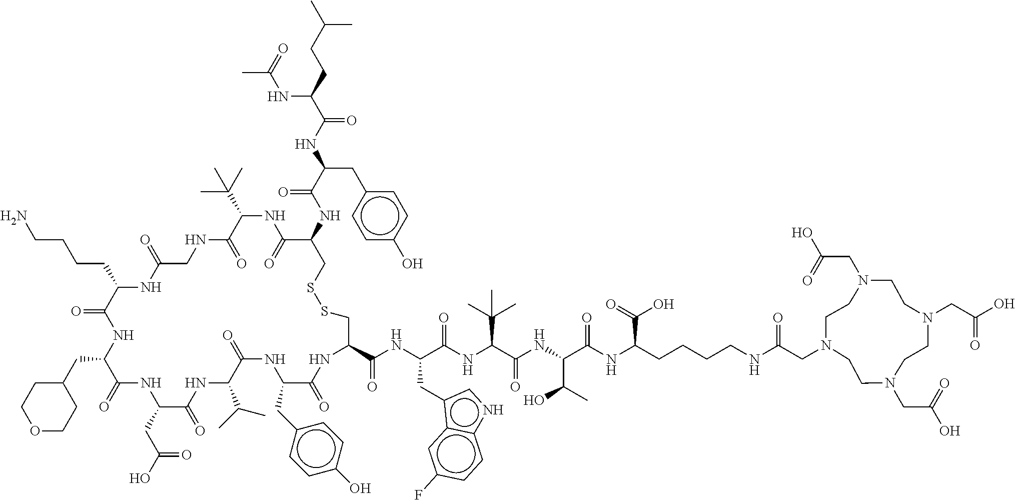 |
| 336 | 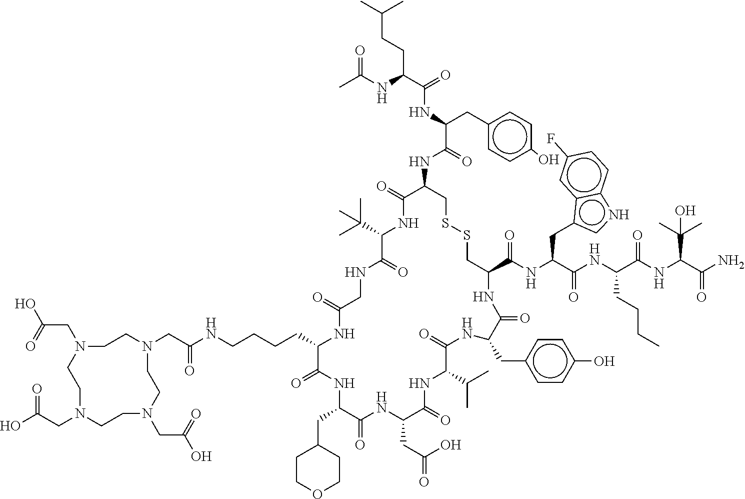 |
| 337 | 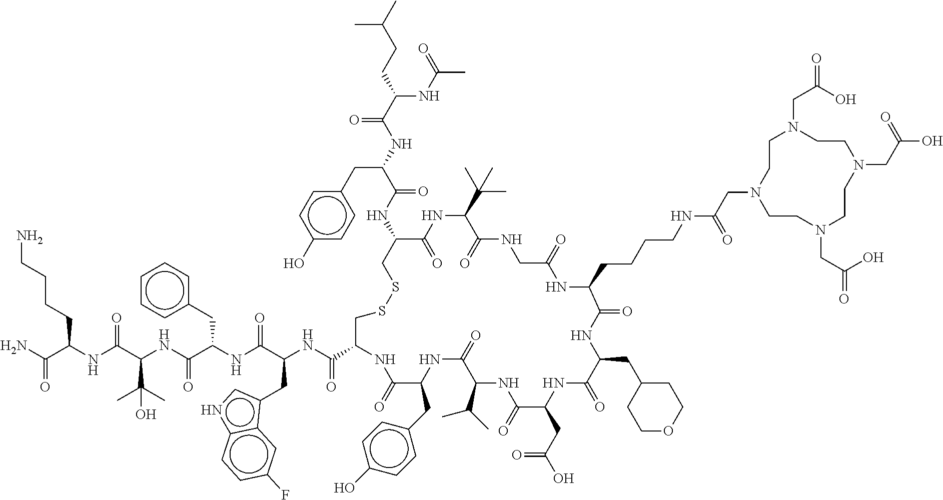 |
| 338 | 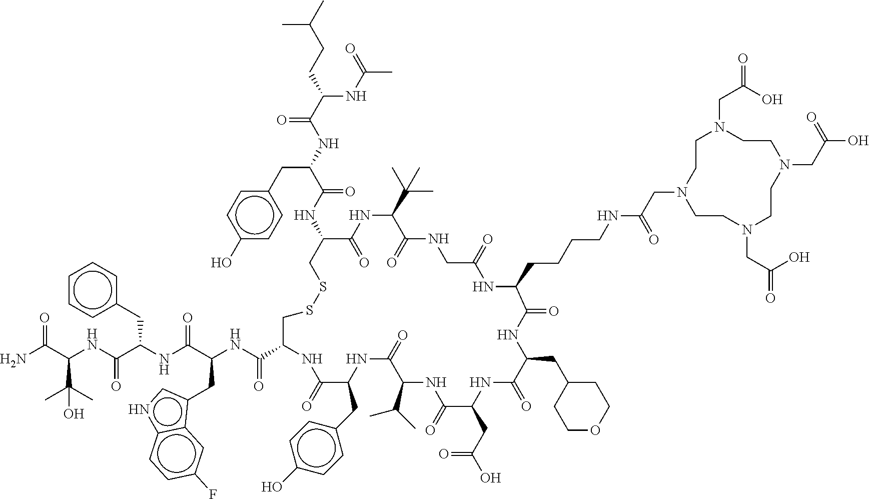 |
| 339 | 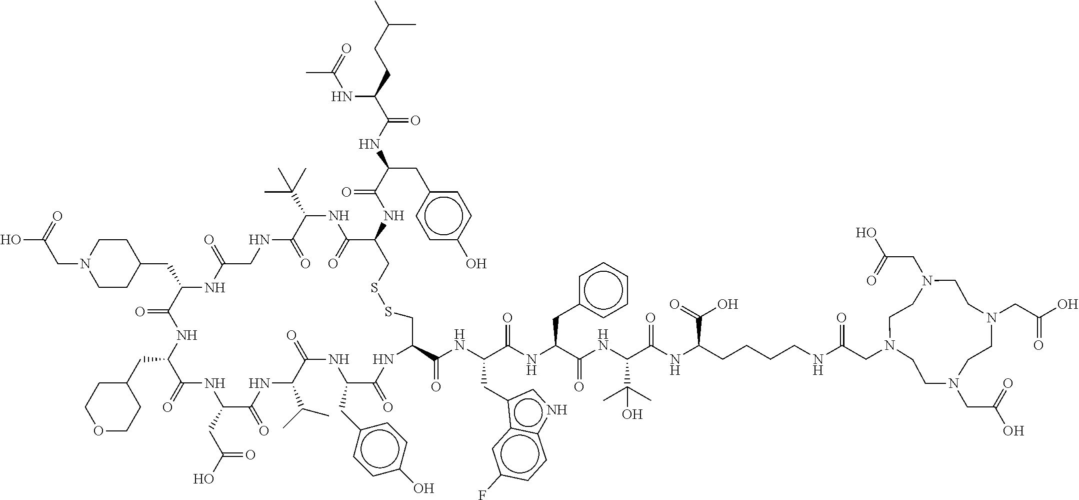 |
| 340 | 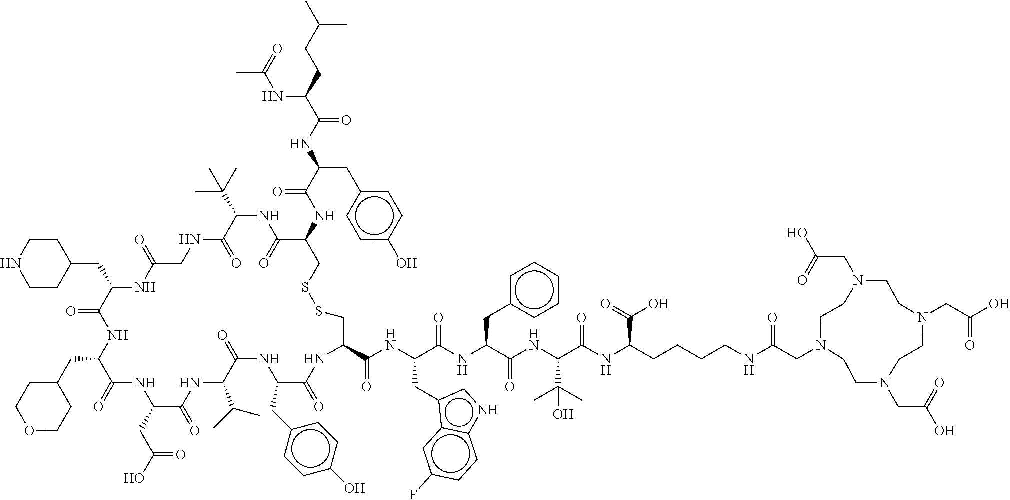 |
| 341 | 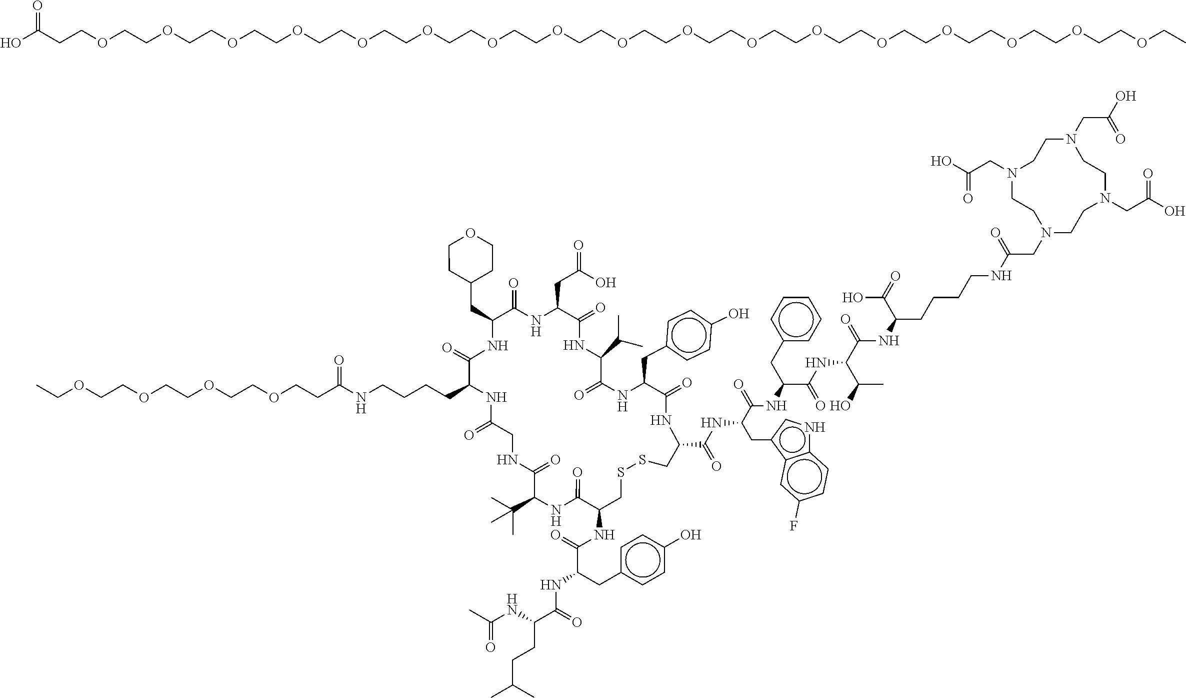 |
| 342 | 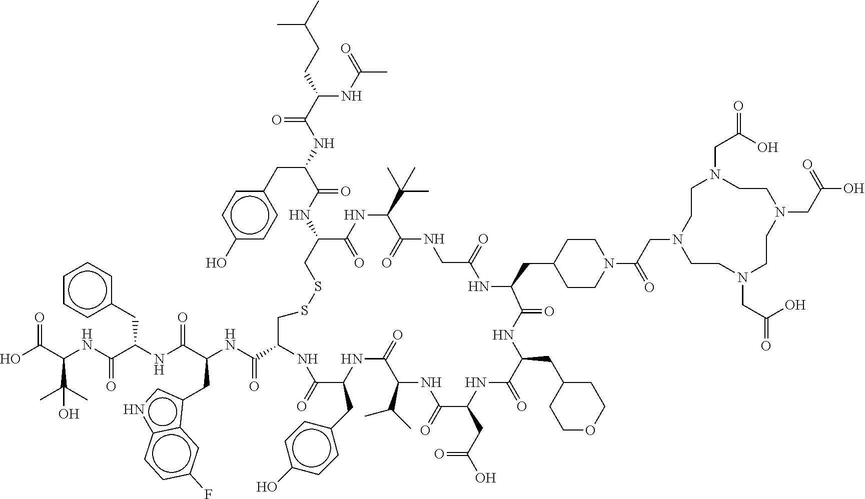 |
| 343 | 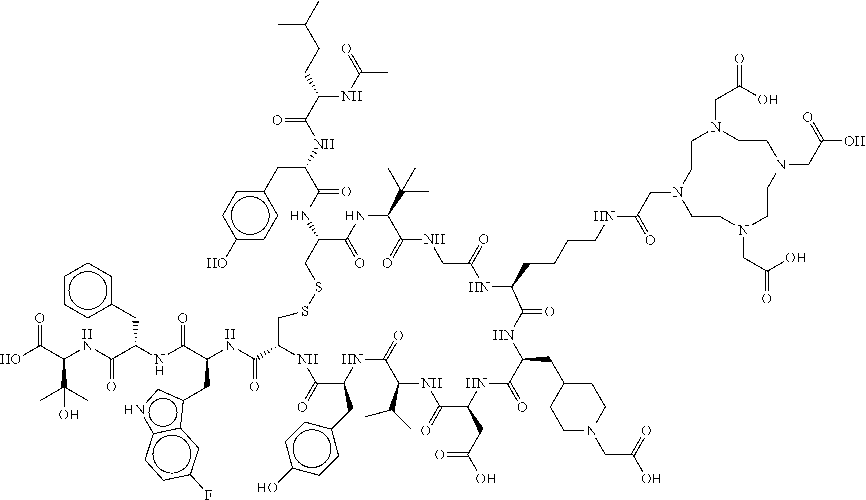 |
| 344 | 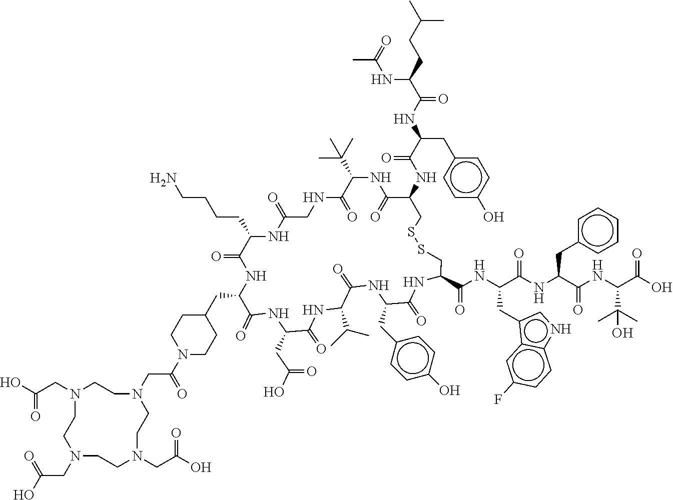 |
| 345 | 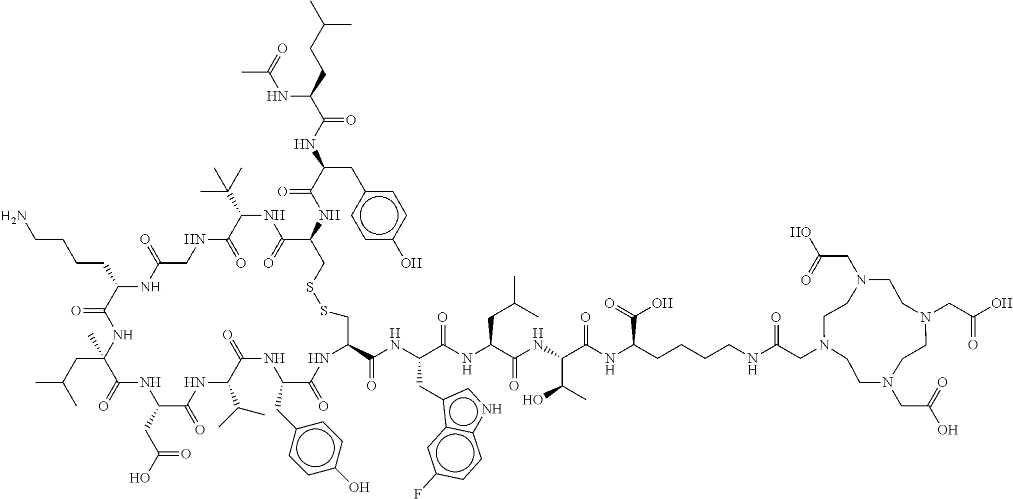 |
| 347 | 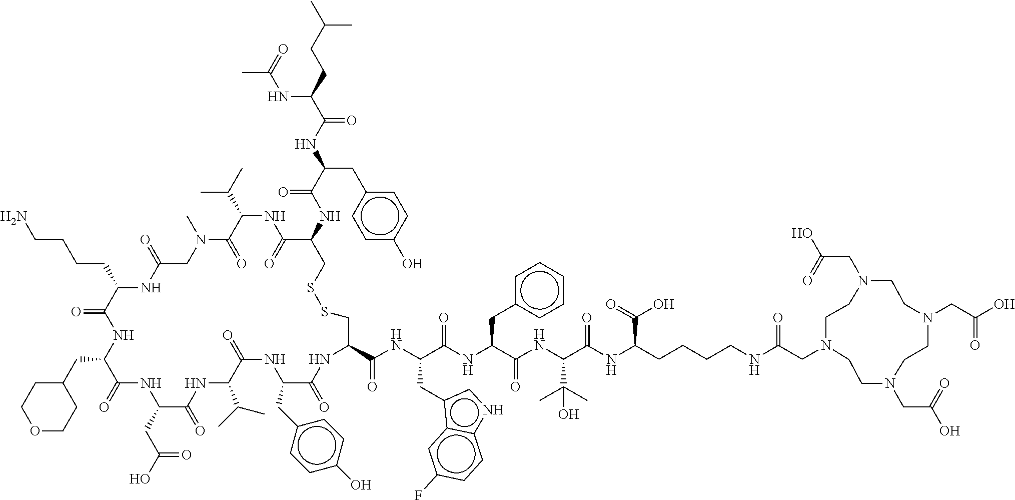 |
| 348 | 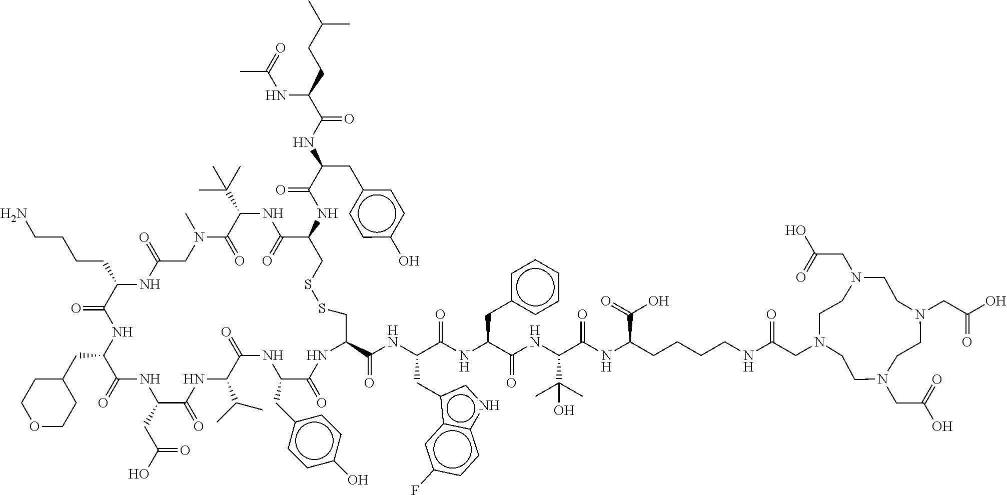 |
| 349 | 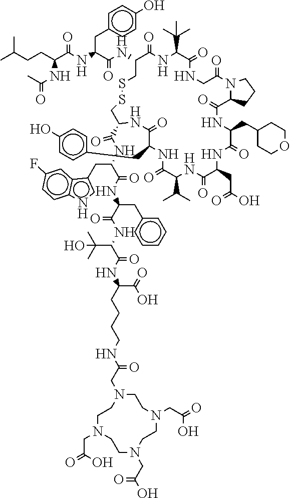 |
| 350 | 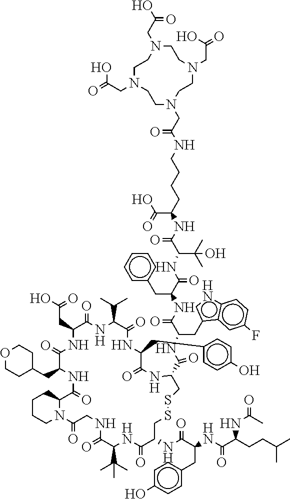 |
| 351 | 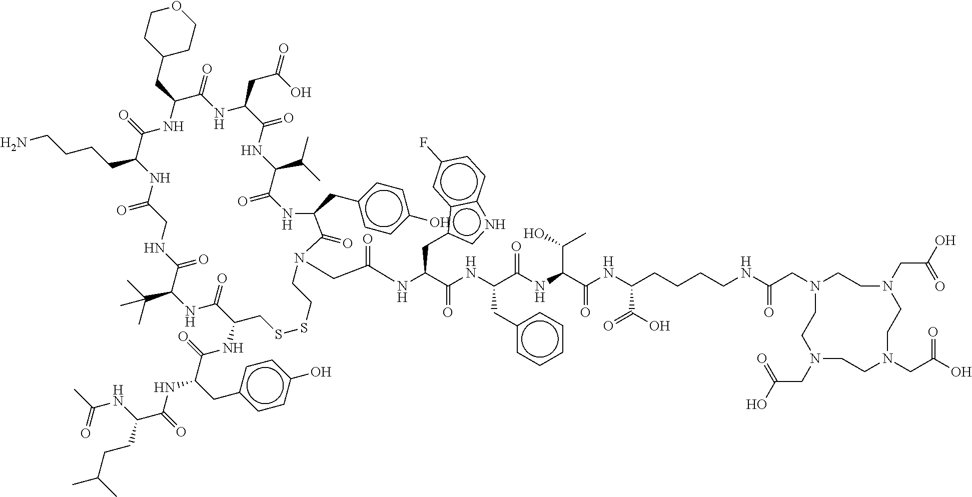 |
| 352 | 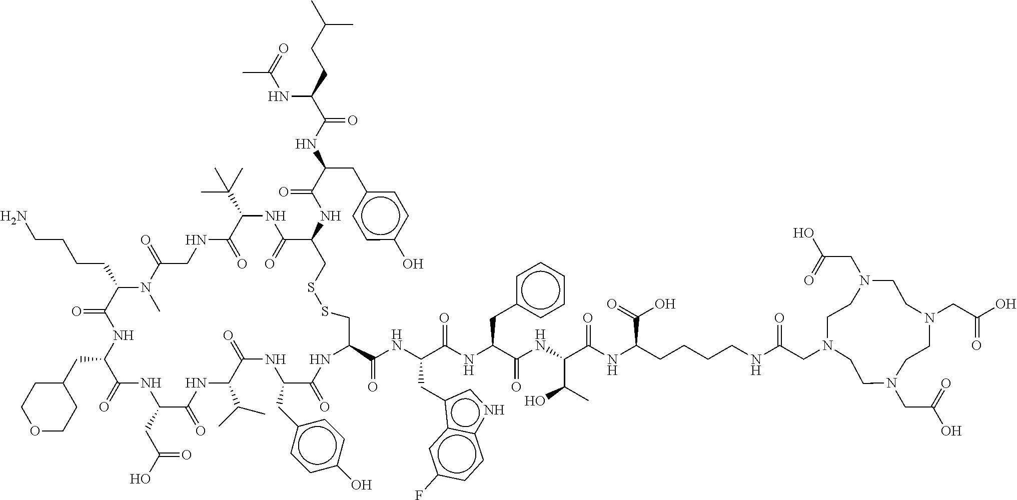 |
| 353 | 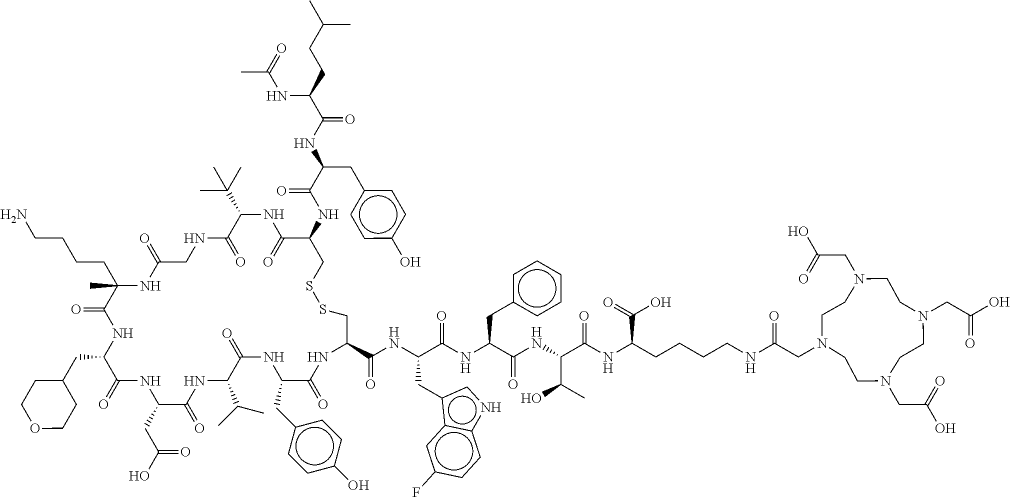 |
| 354 | 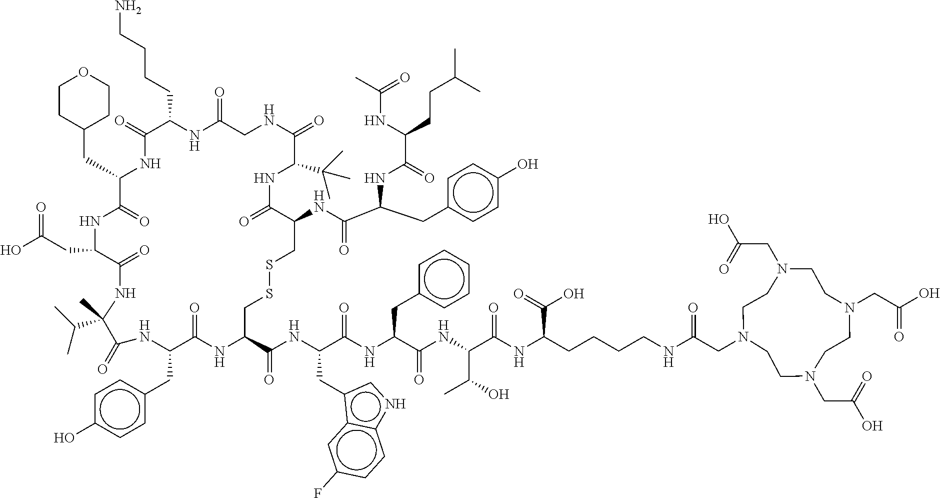 |
| 355 | 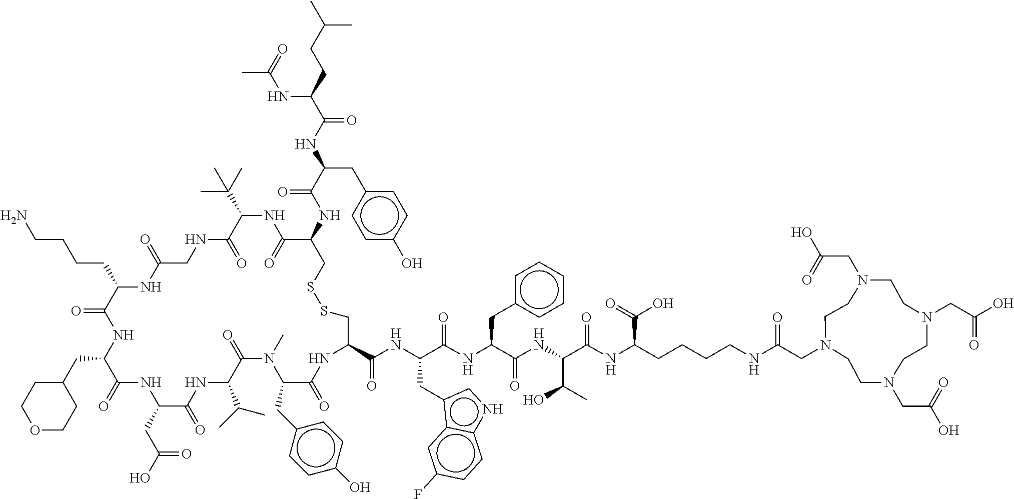 |
| 356 | 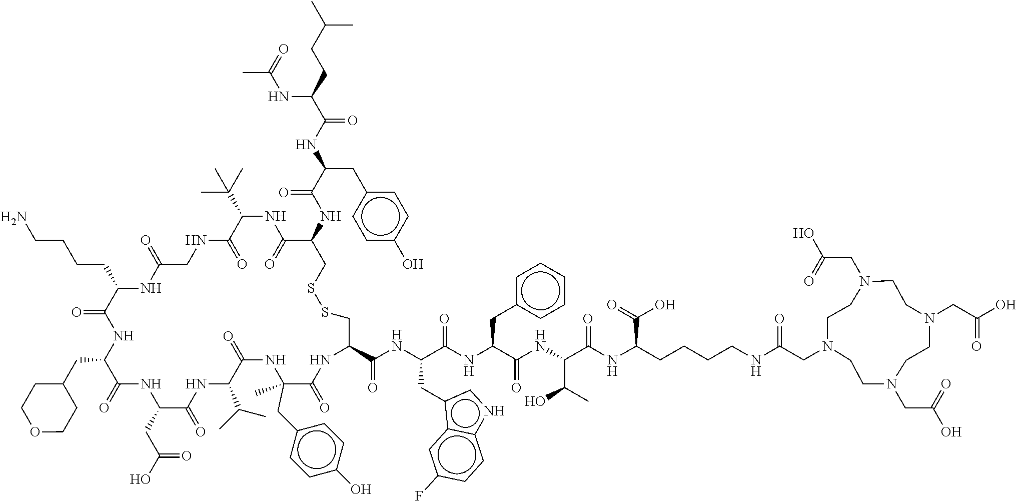 |
| 357 | 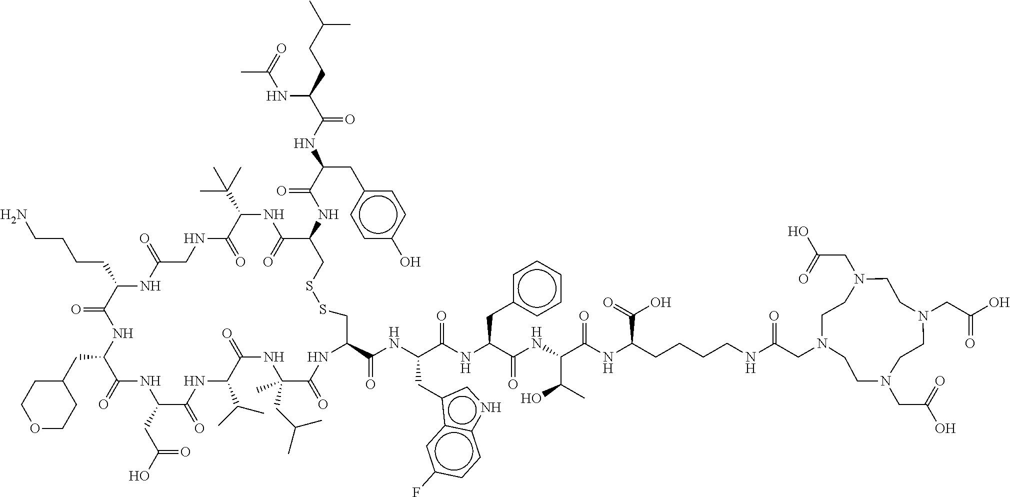 |
| 358 | 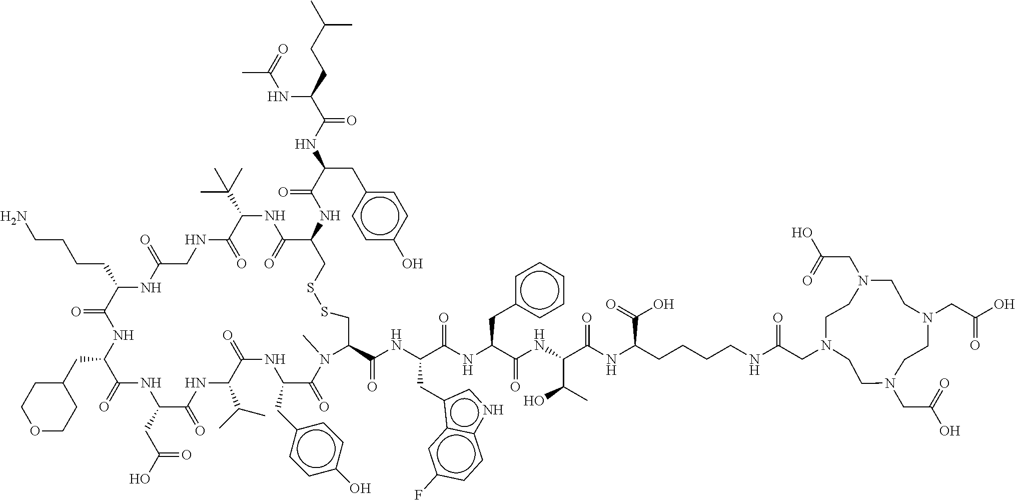 |
| 359 | 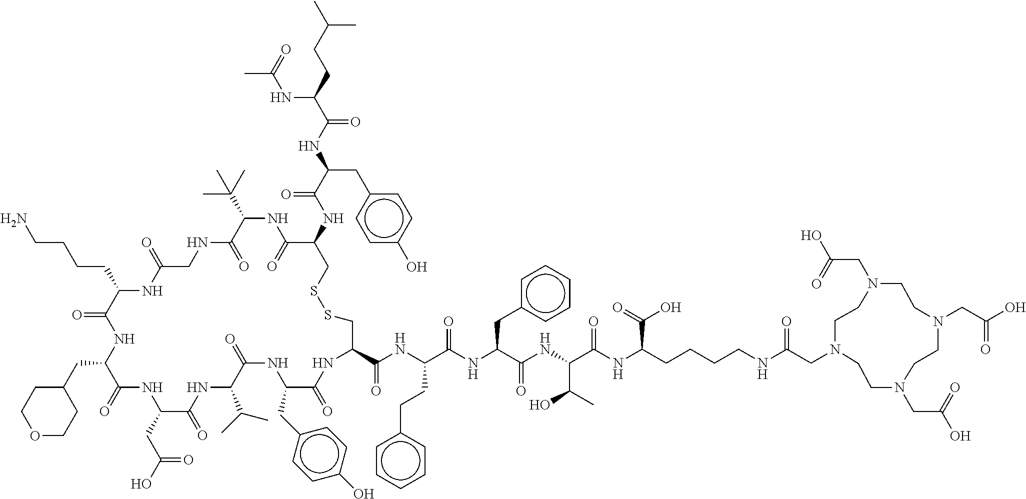 |
| 360 | 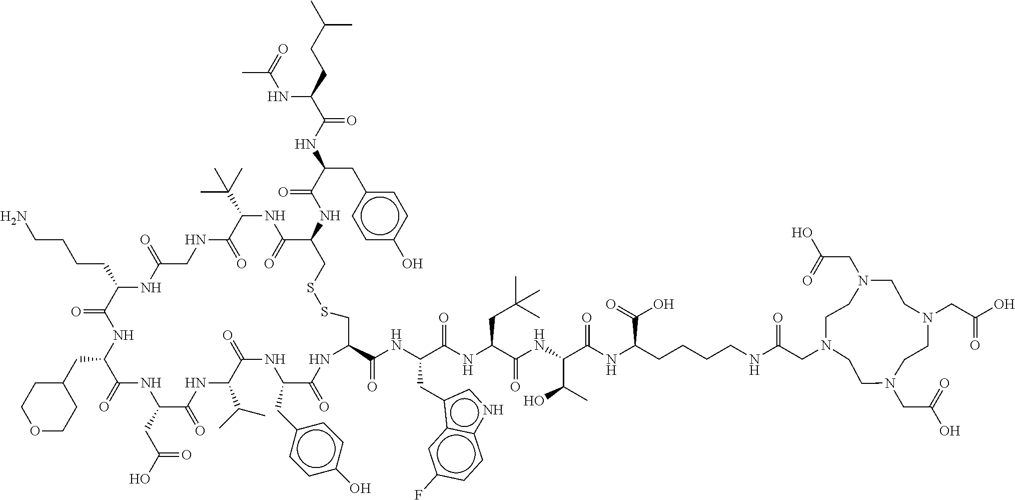 |
| 361 | 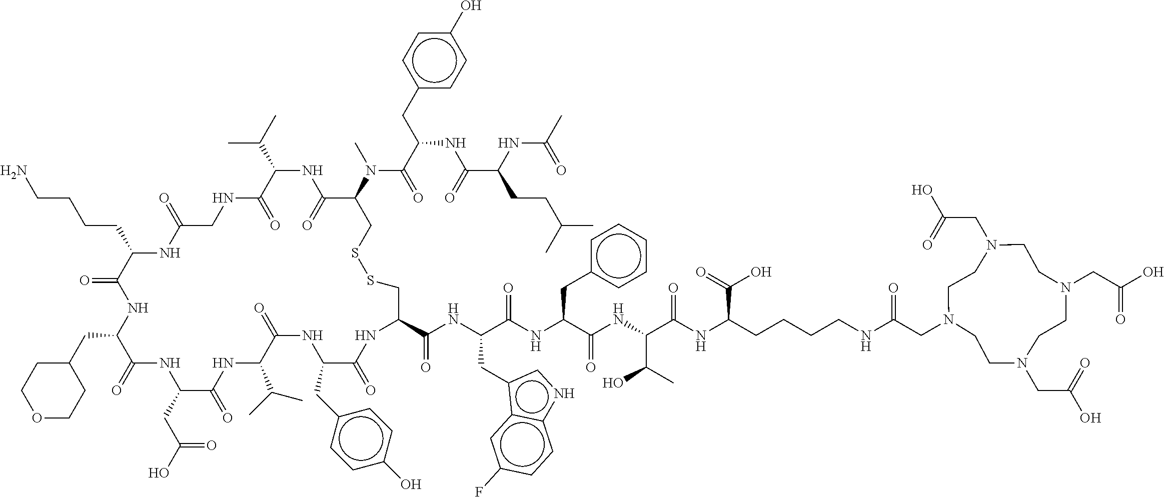 |
| 362 | 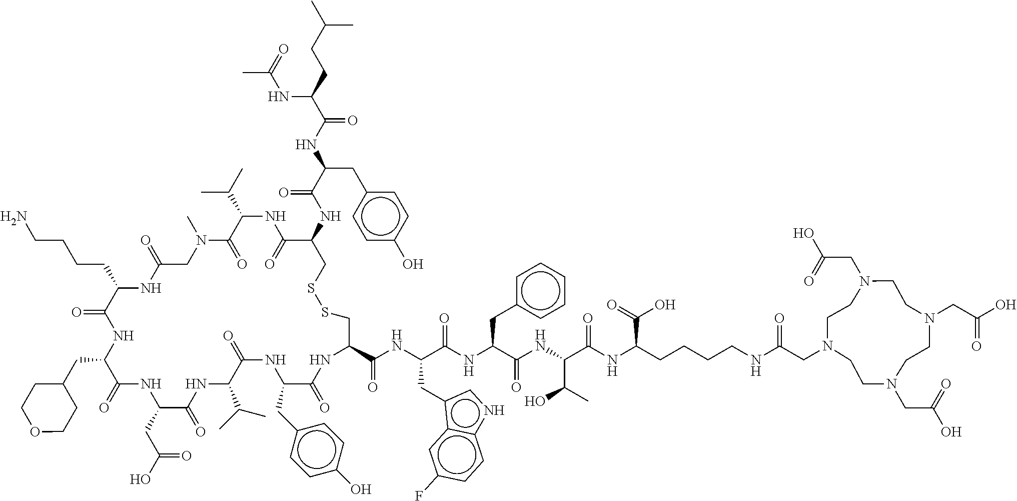 |
| 363 | 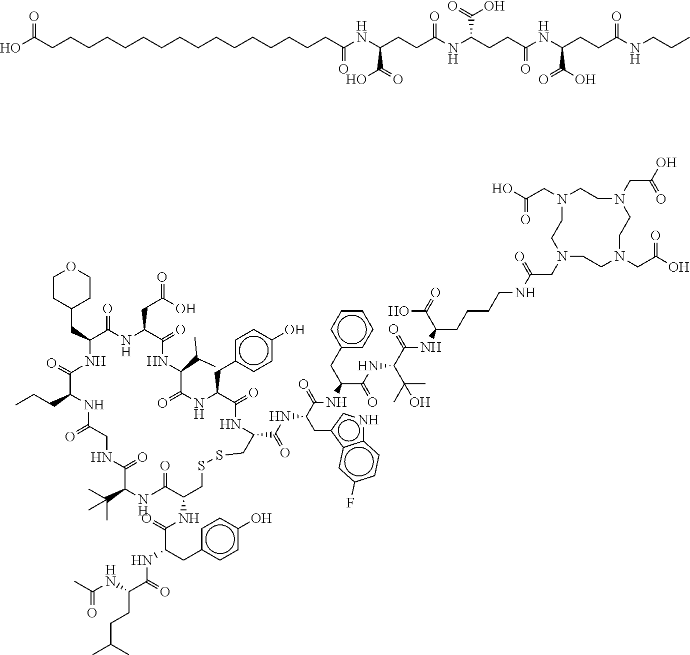 |
| 364 | 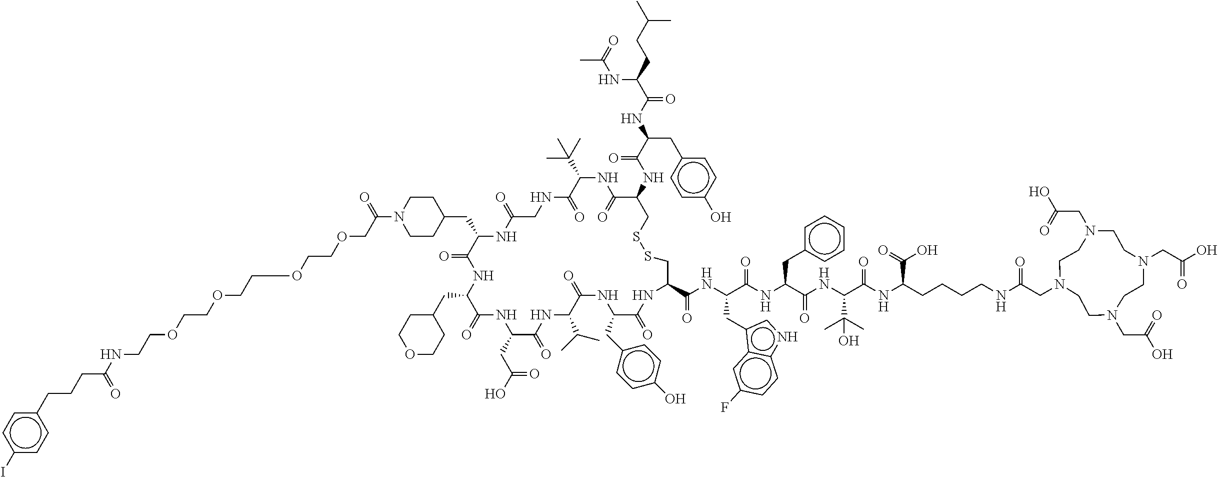 |
| 365 | 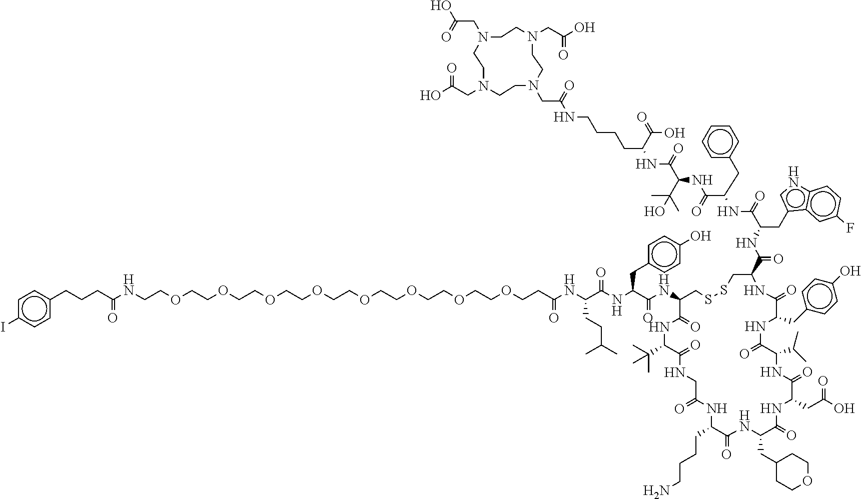 |
| 366 | 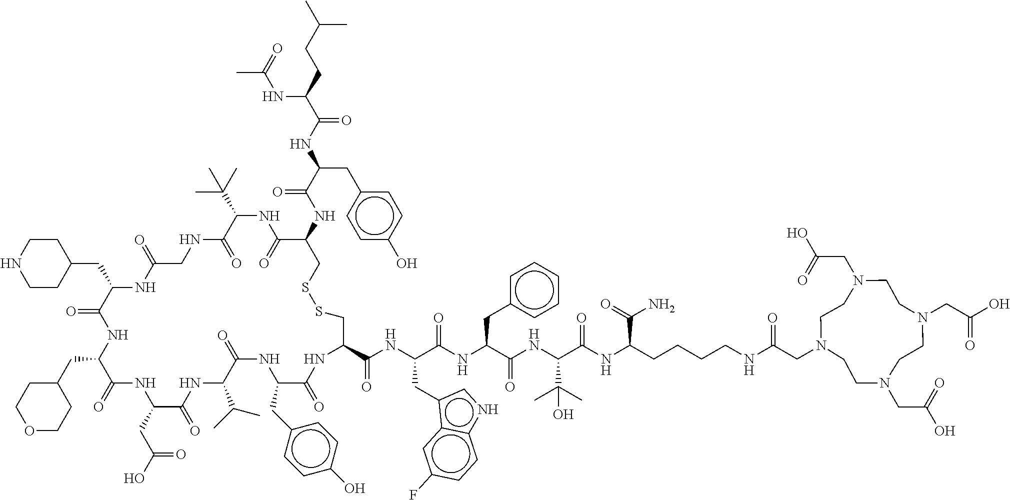 |
| 367 | 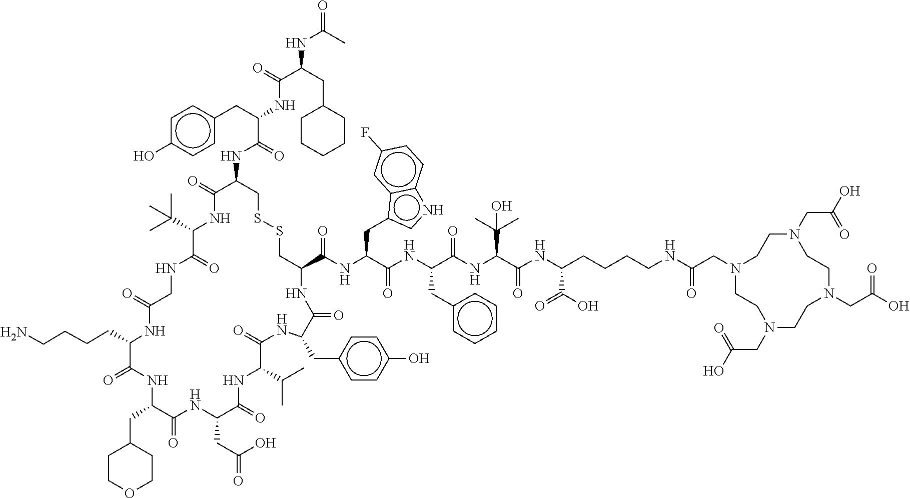 |
| 368 | 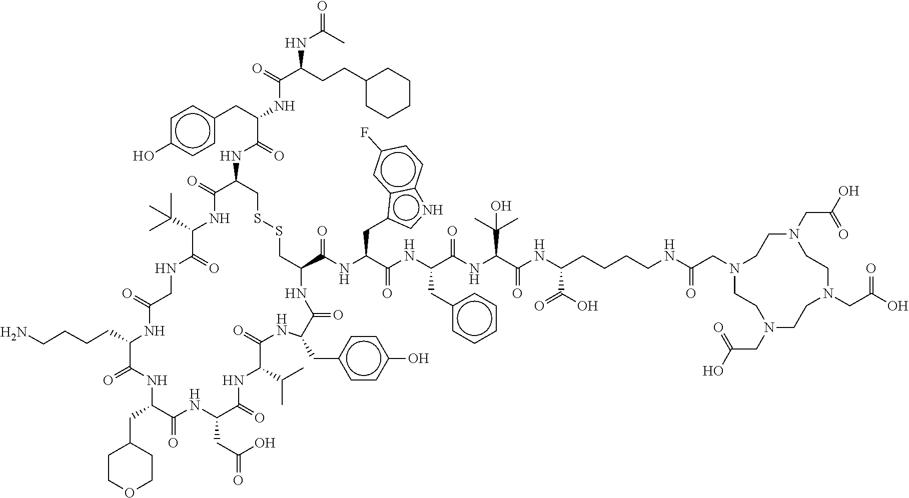 |
| 369 | 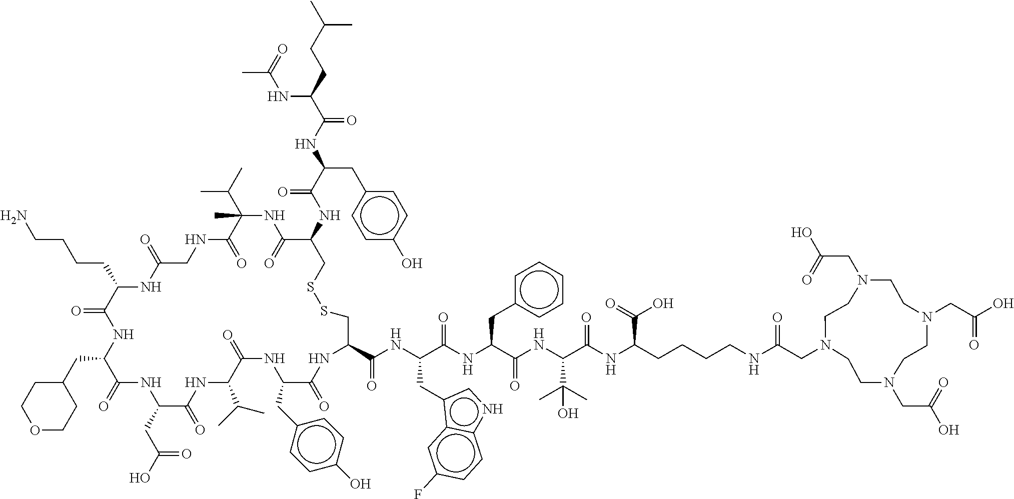 |
| 370 | 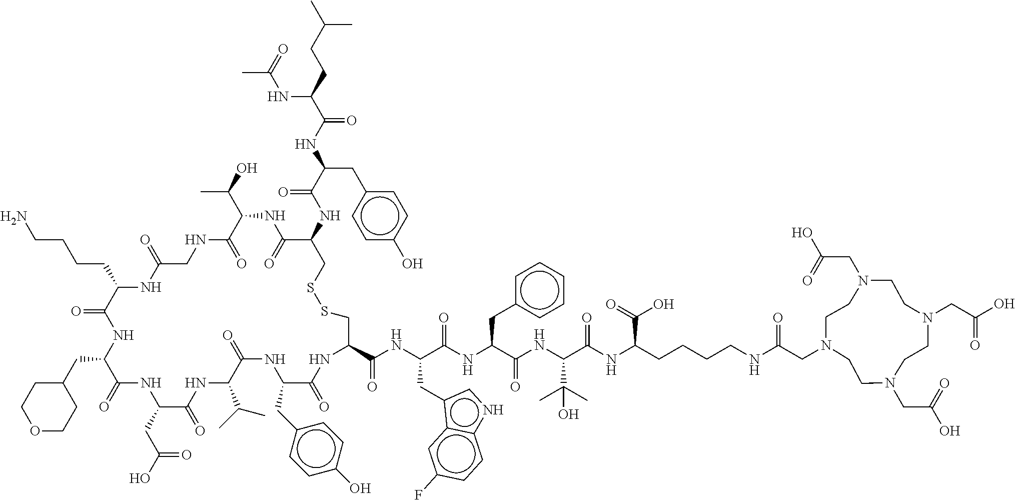 |
| 371 | 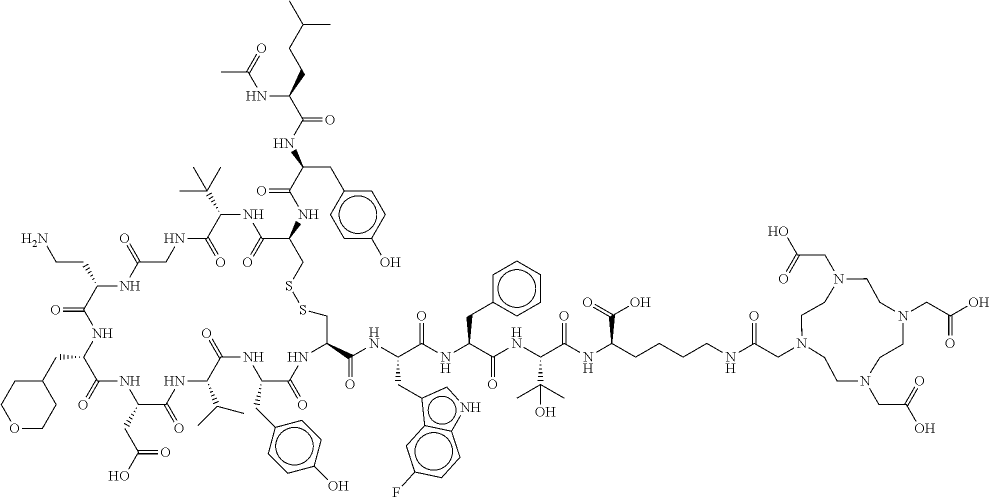 |
| 372 | 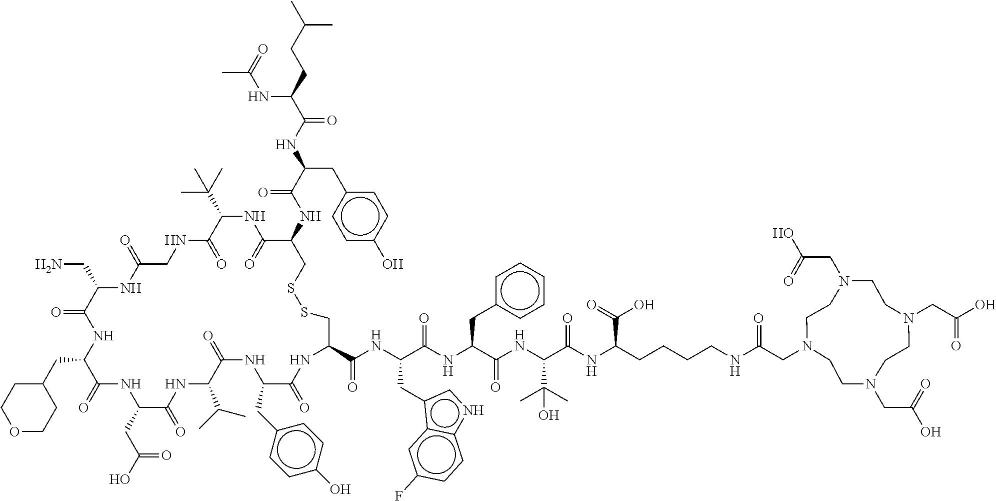 |
| 373 | 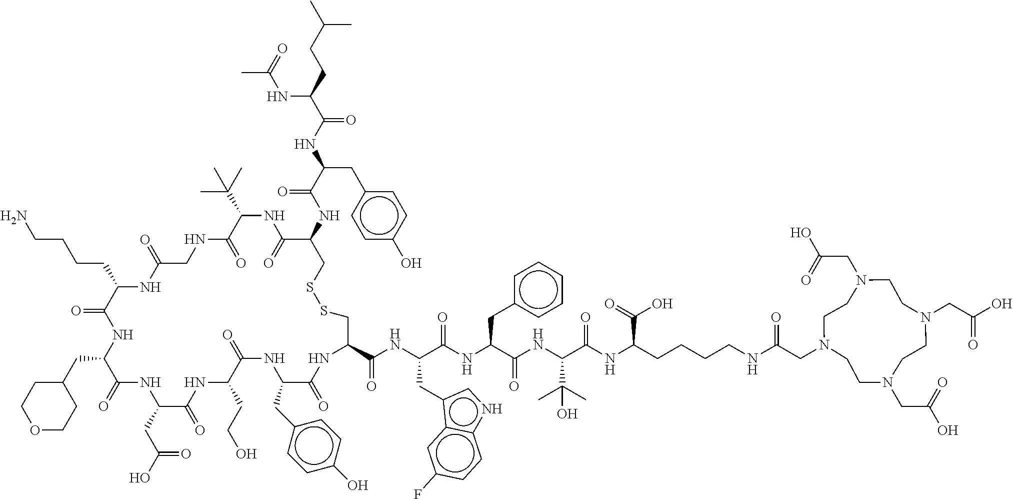 |
| 374 | 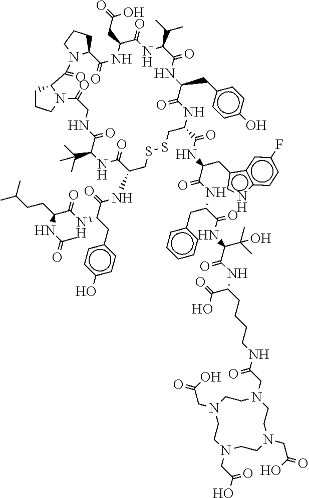 |
| 375 | 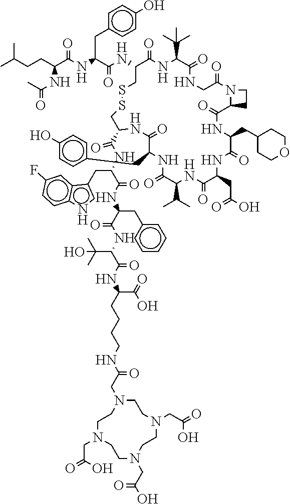 |
| 376 | 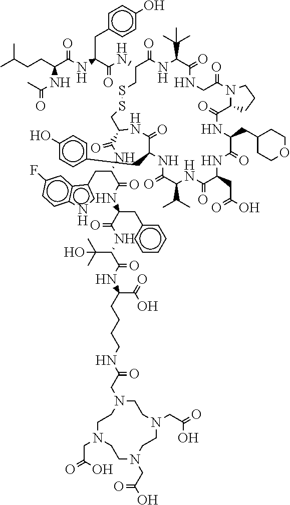 |
| 377 | 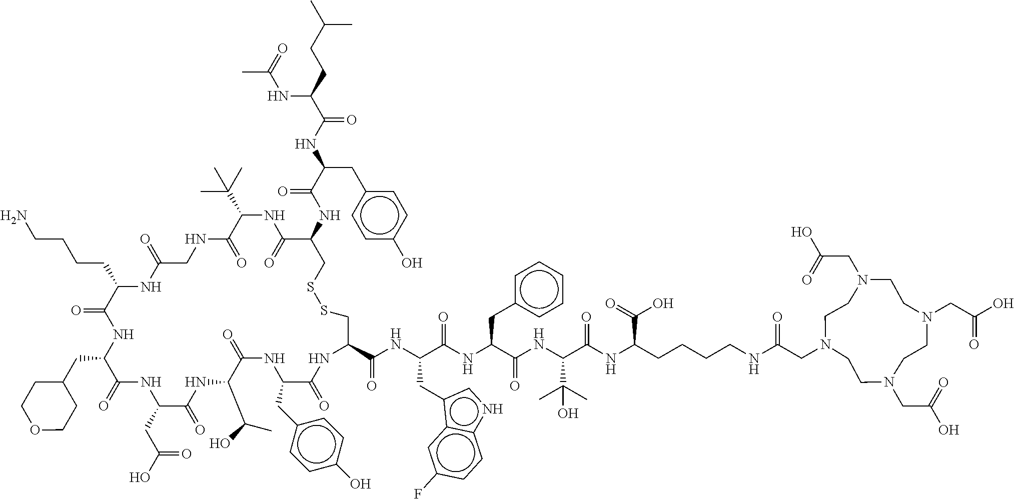 |
| 378 | 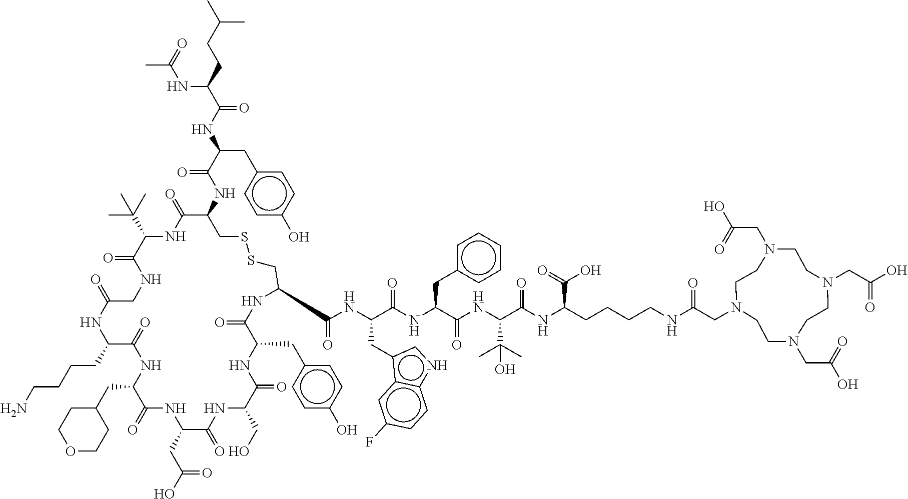 |
| 379 | 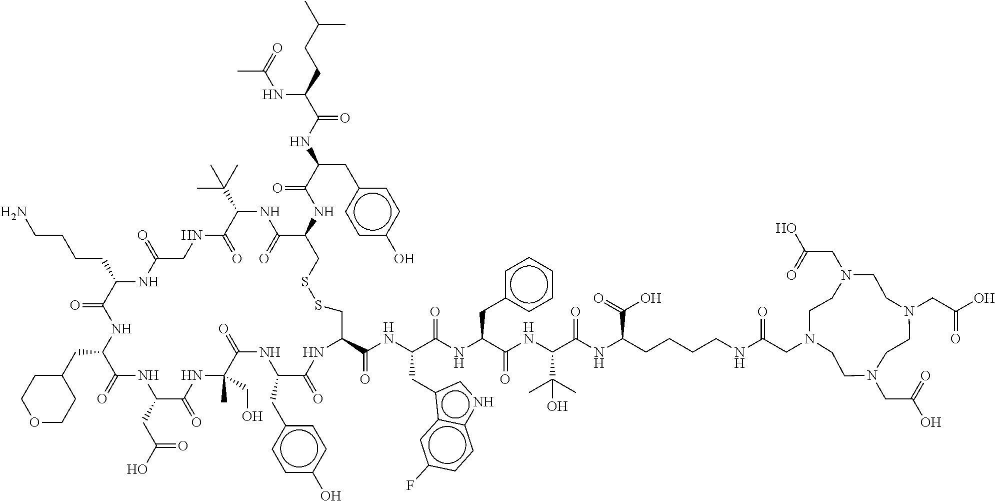 |
| 380 | 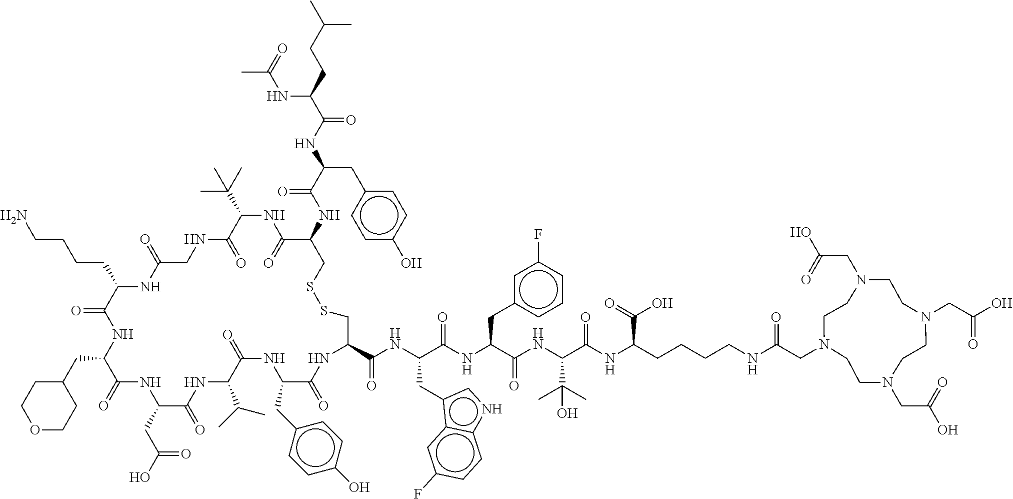 |
| 381 | 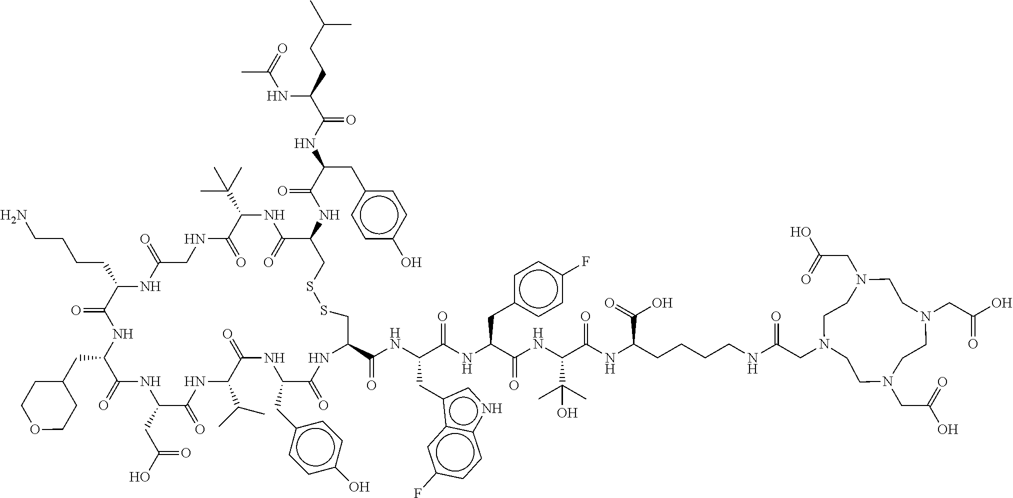 |
| 382 | 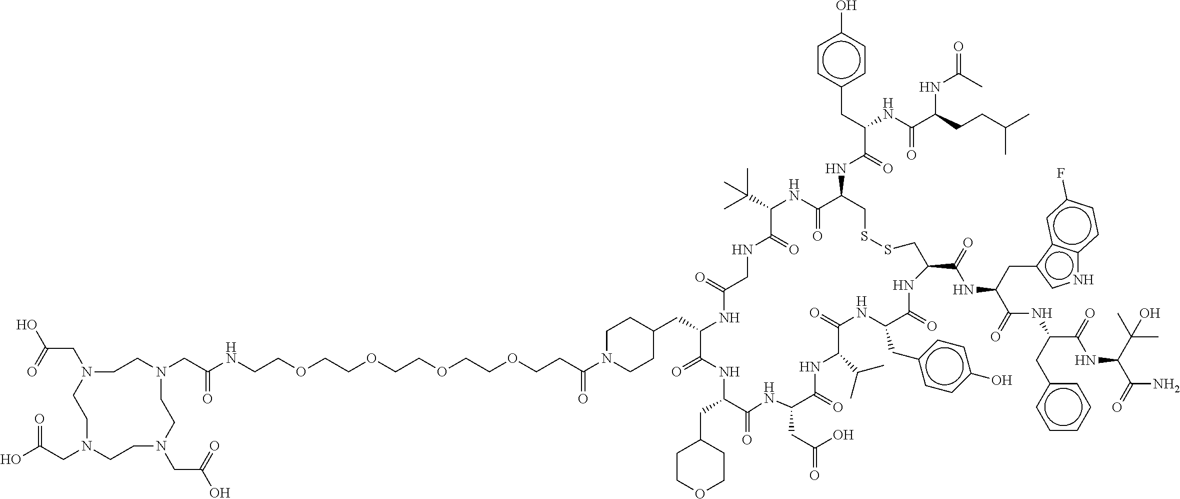 |
| 383 | 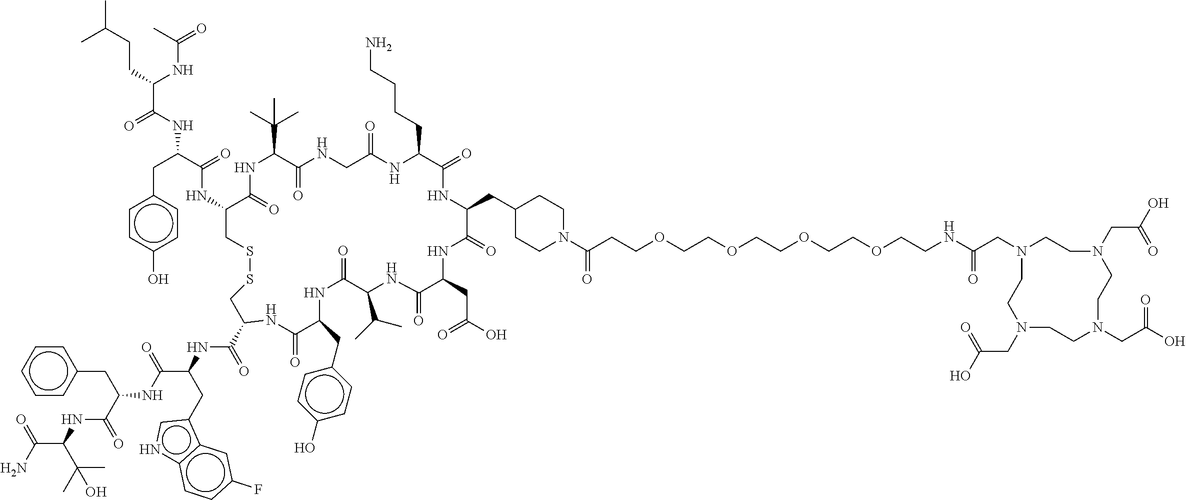 |
| 384 | 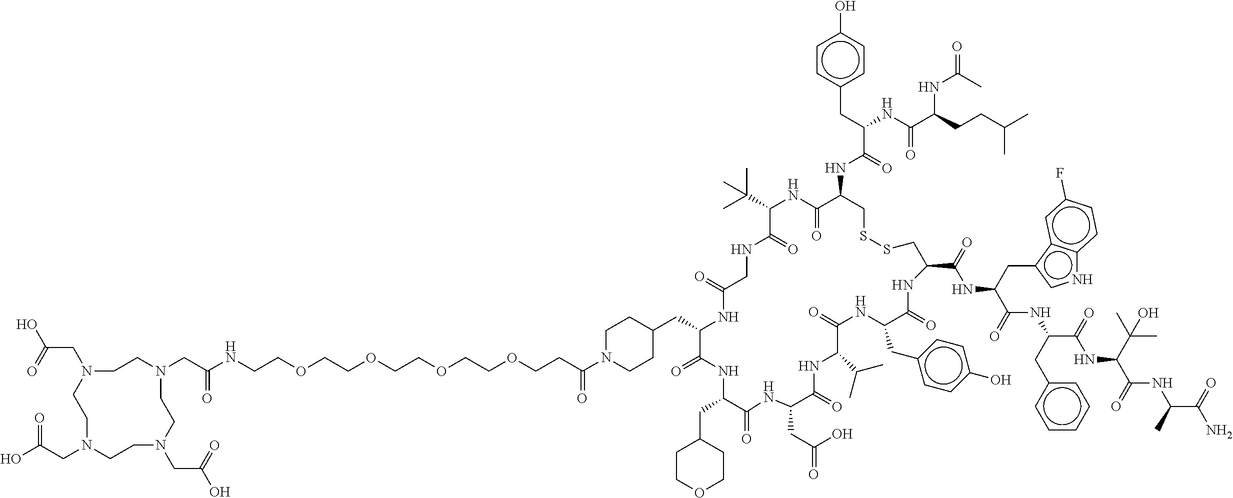 |
| 385 | 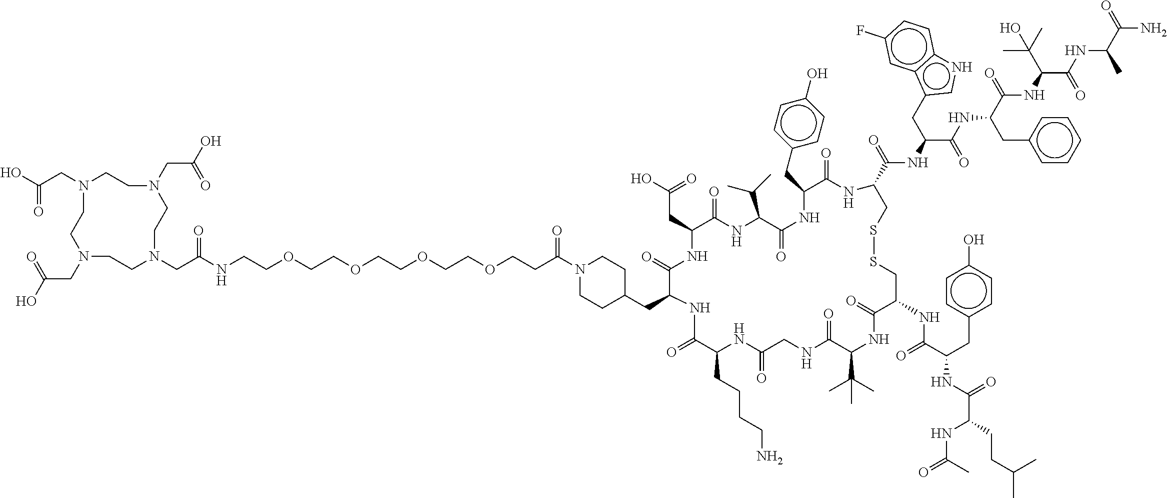 |
| 386 | 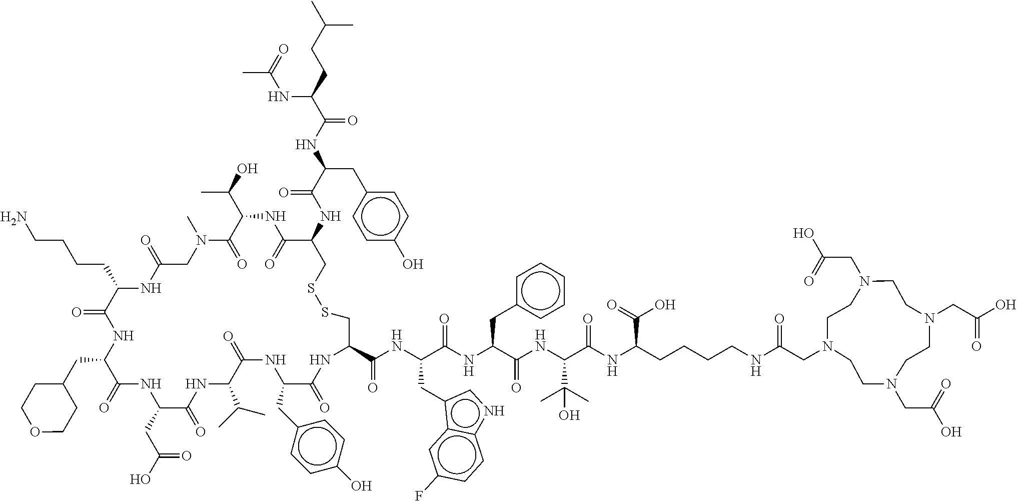 |
| 389 | 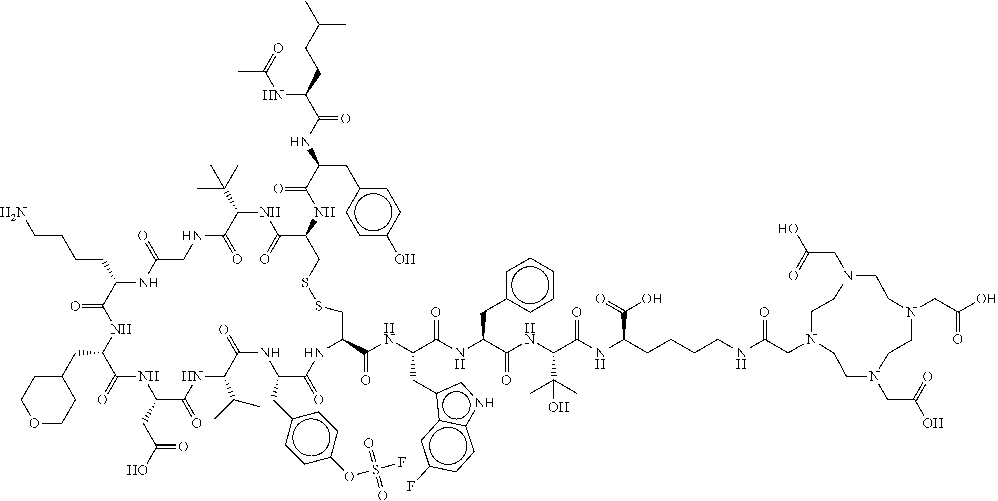 |
| 390 | 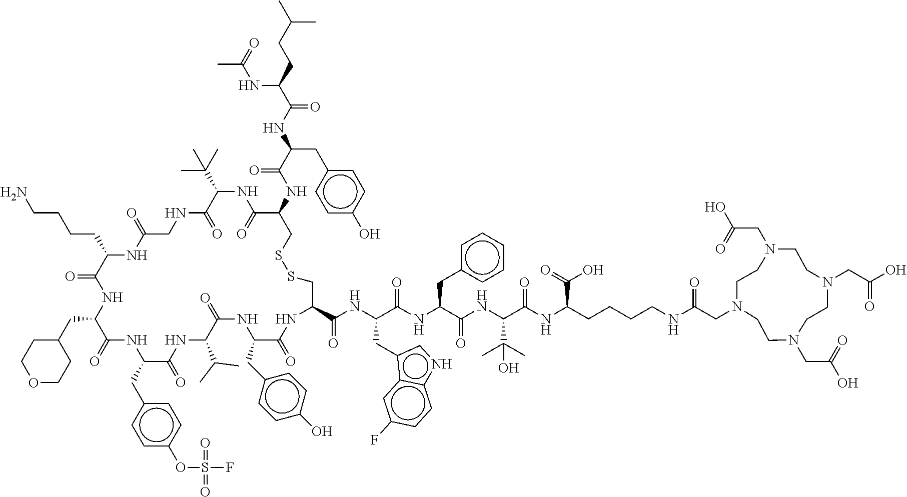 |
| 391 | 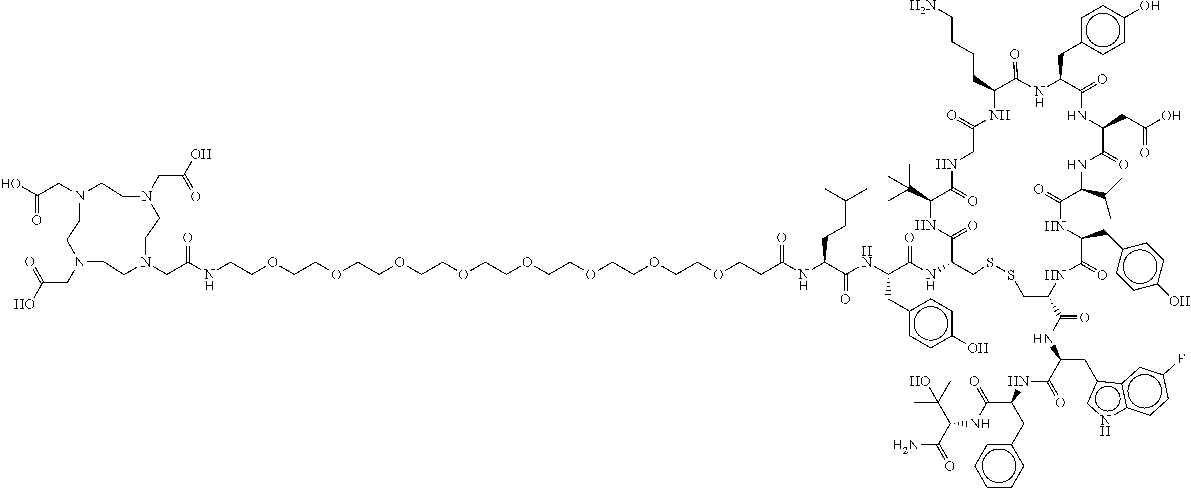 |
| 392 | 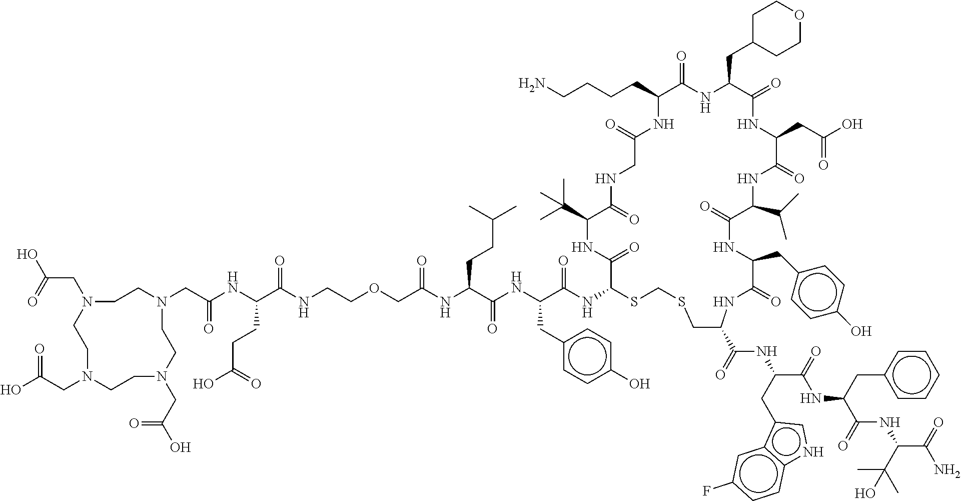 |
| 393 | 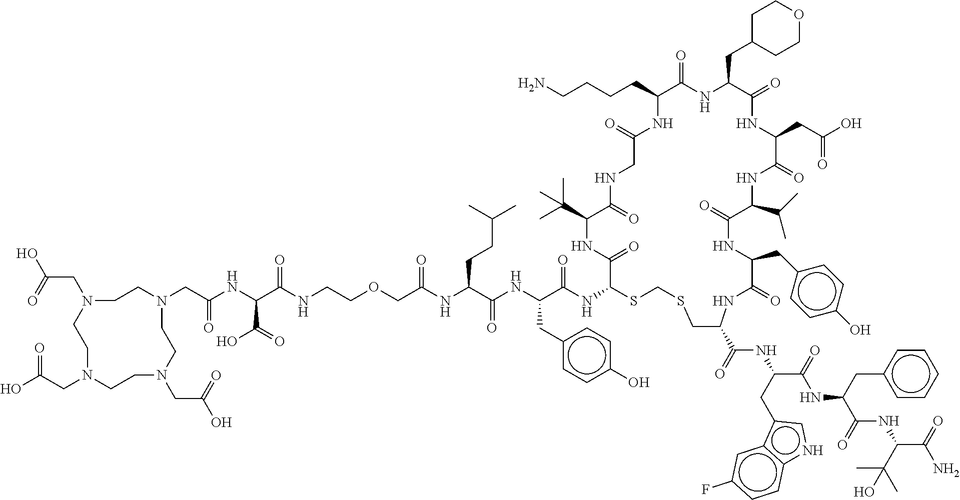 |
| 394 | 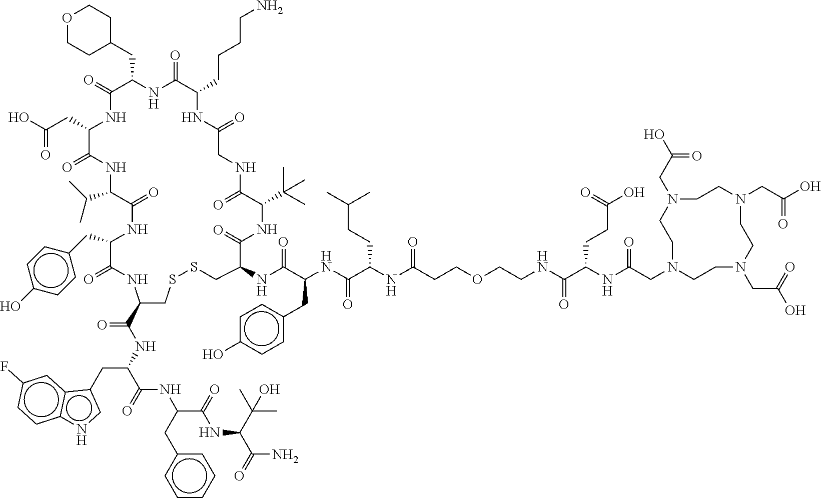 |
| 395 | 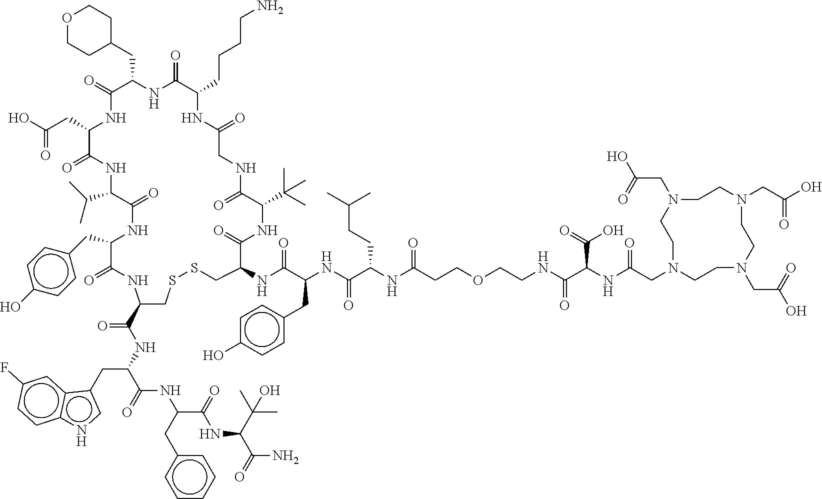 |
| 396 | 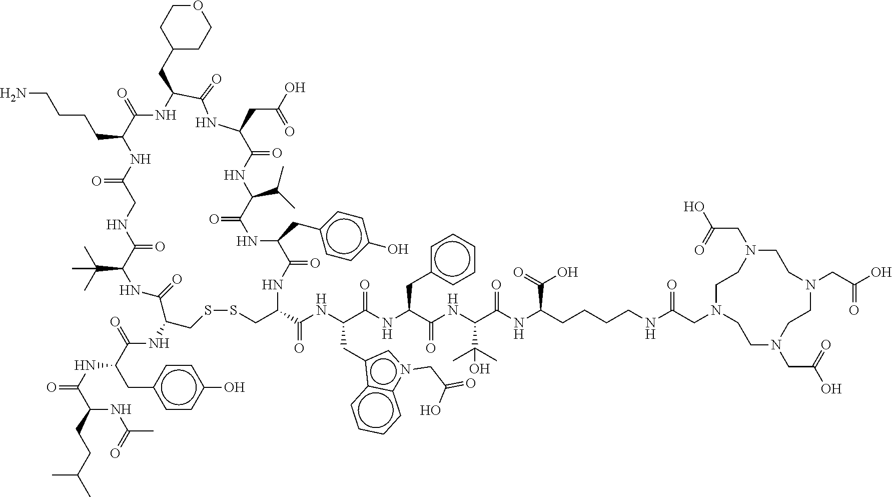 |
| 397 | 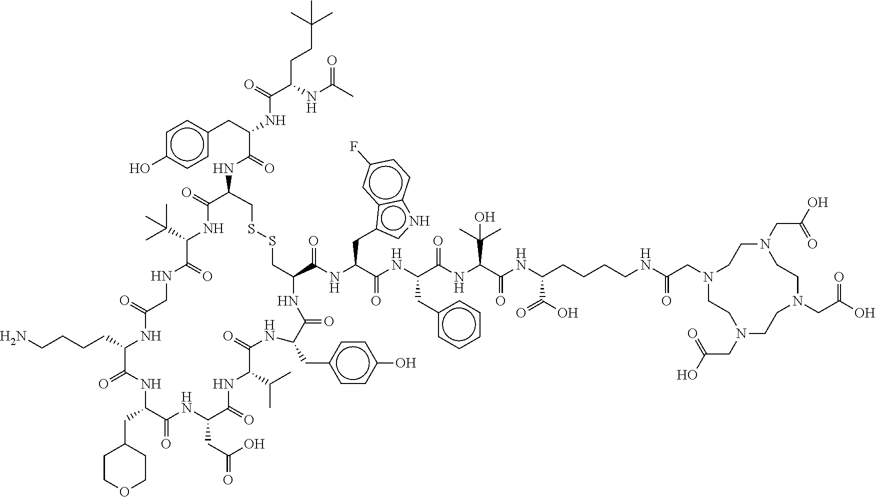 |
| 398 | 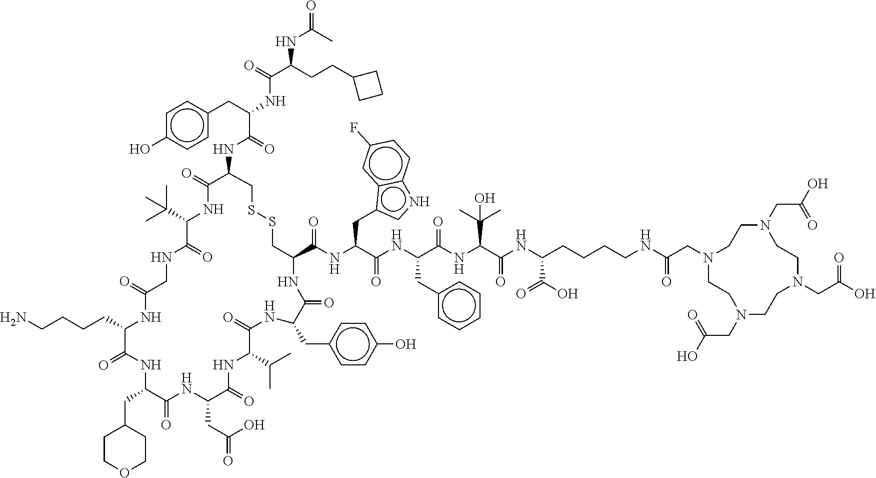 |
| 399 | 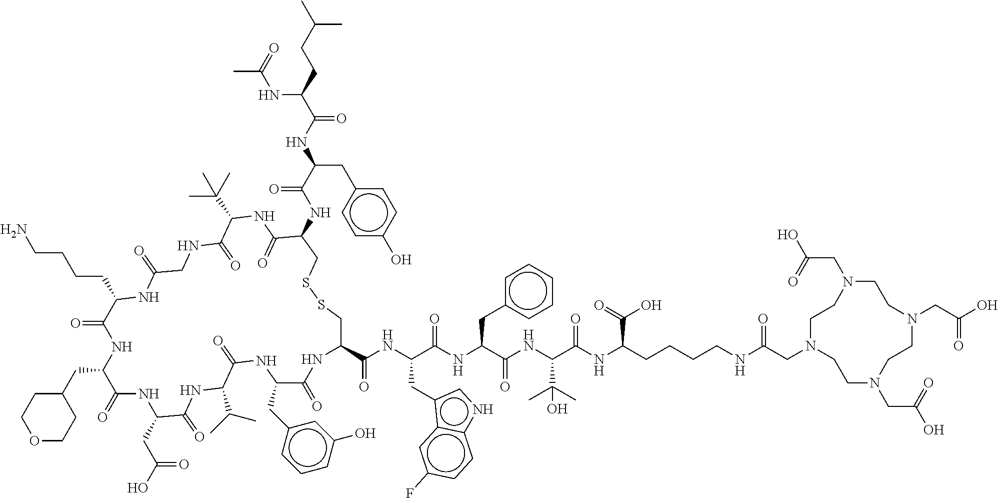 |
| 400 | 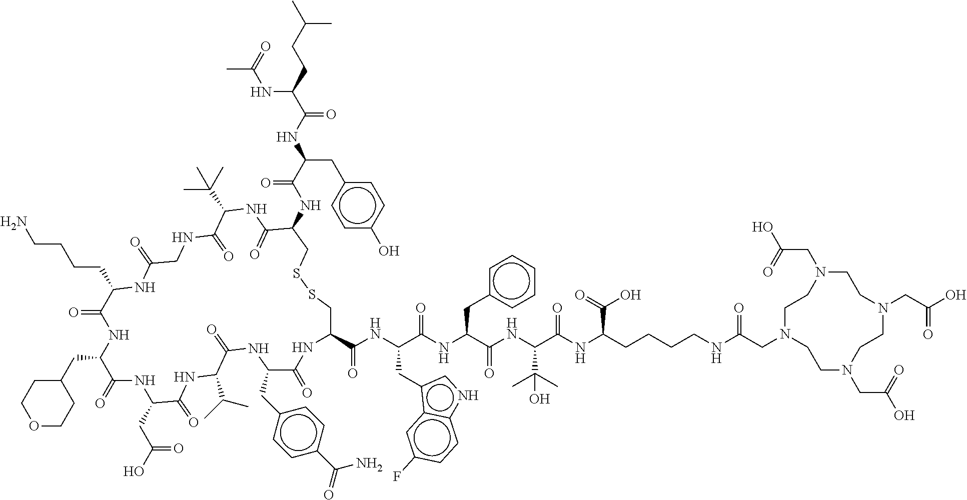 |
| 401 | 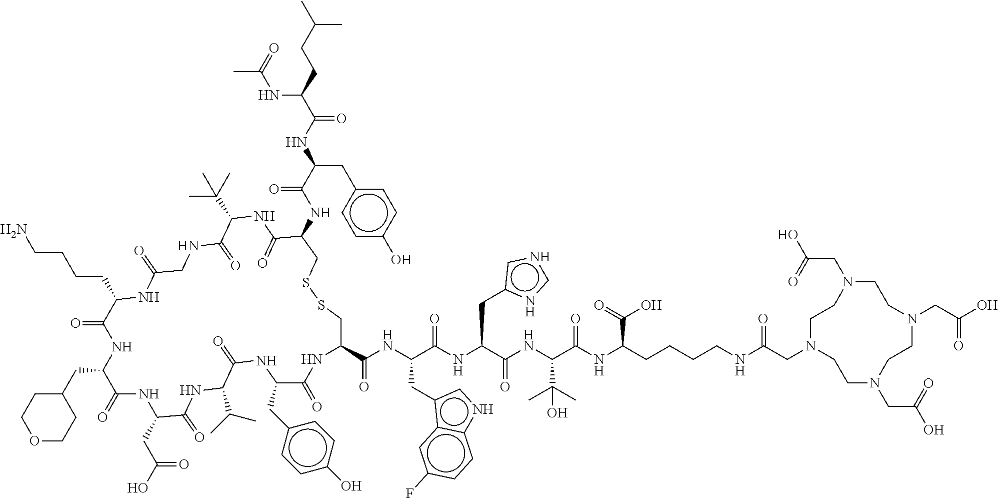 |
| 402 | 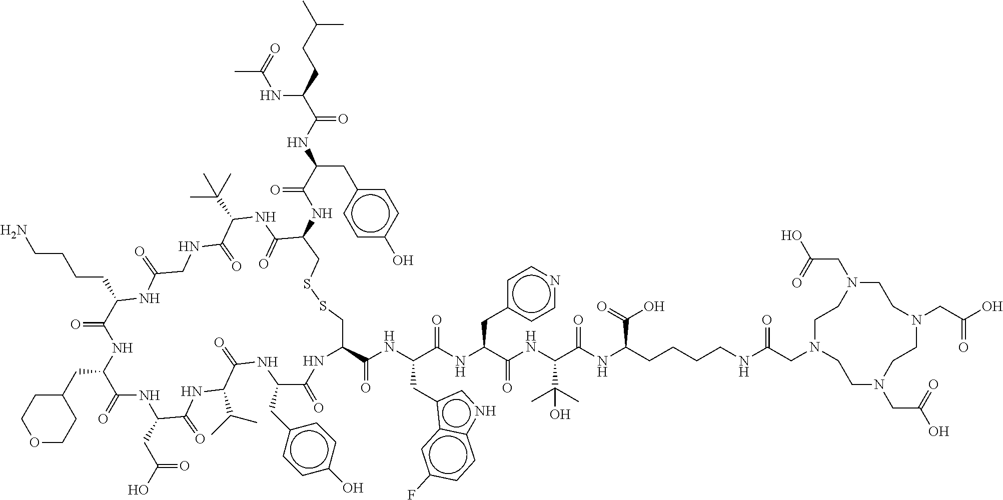 |
| 403 | 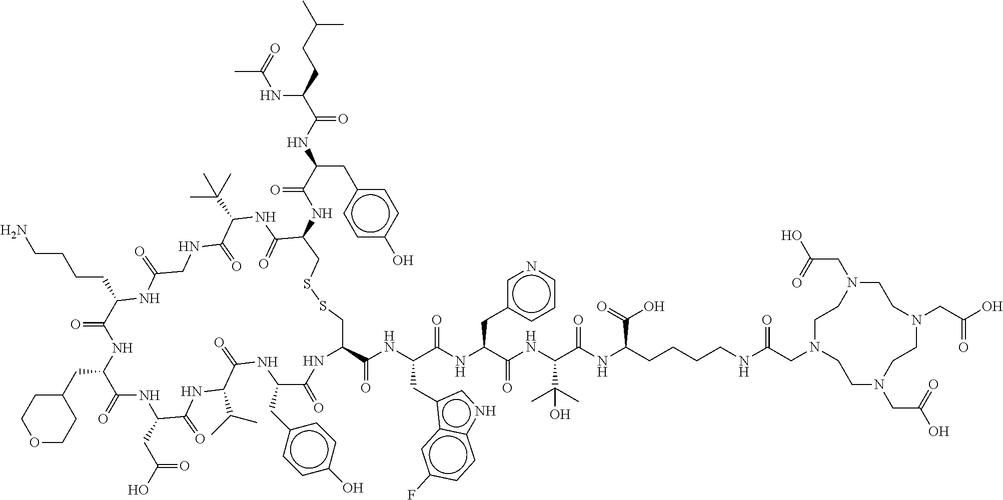 |
| 404 | 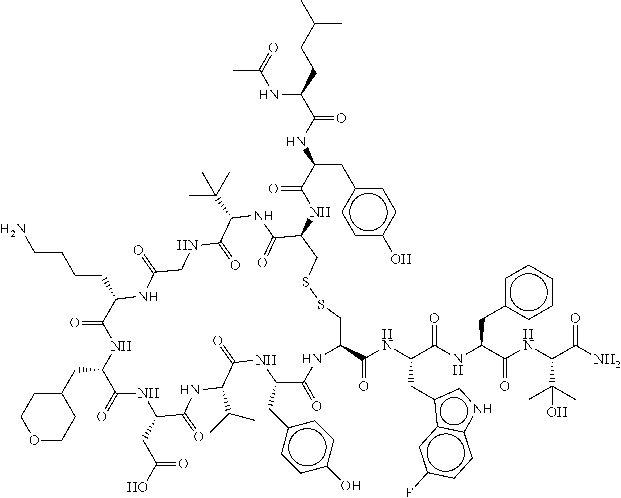 |
| 405 | 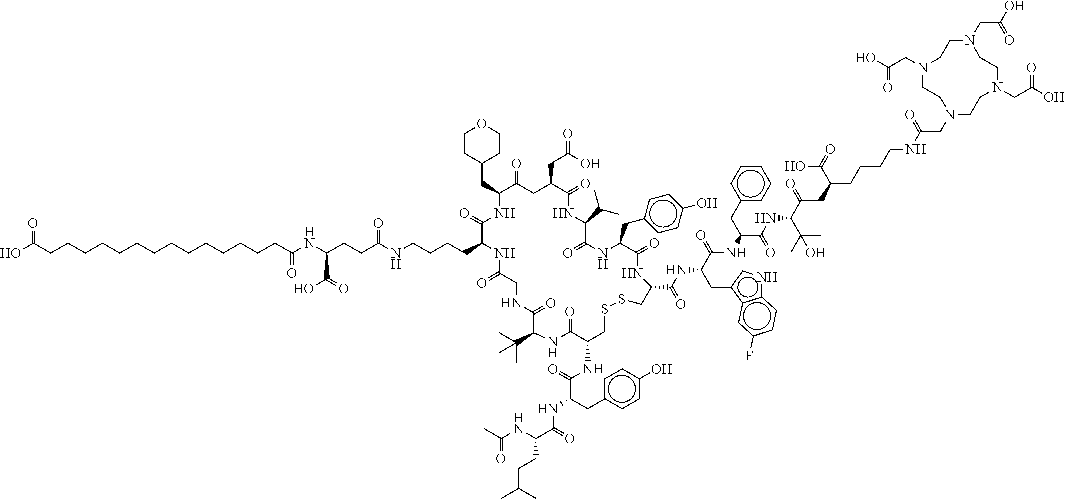 |
| 407 | 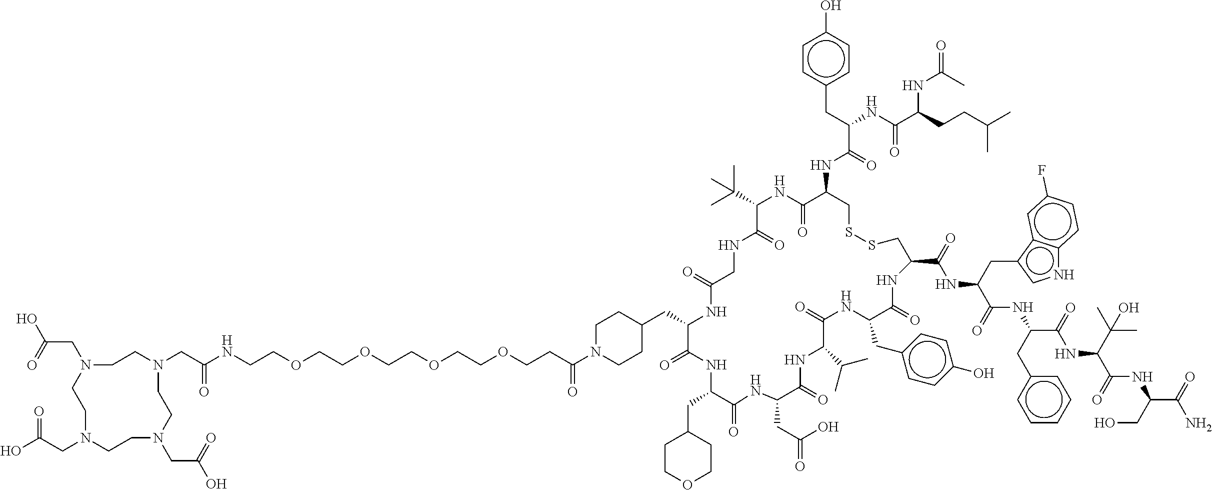 |
| 408 | 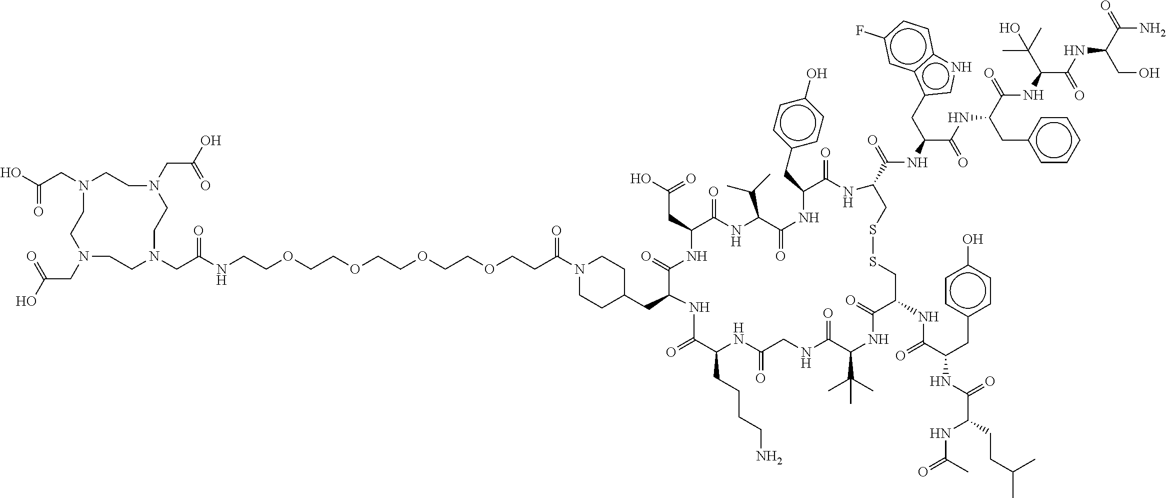 |
| 409 | 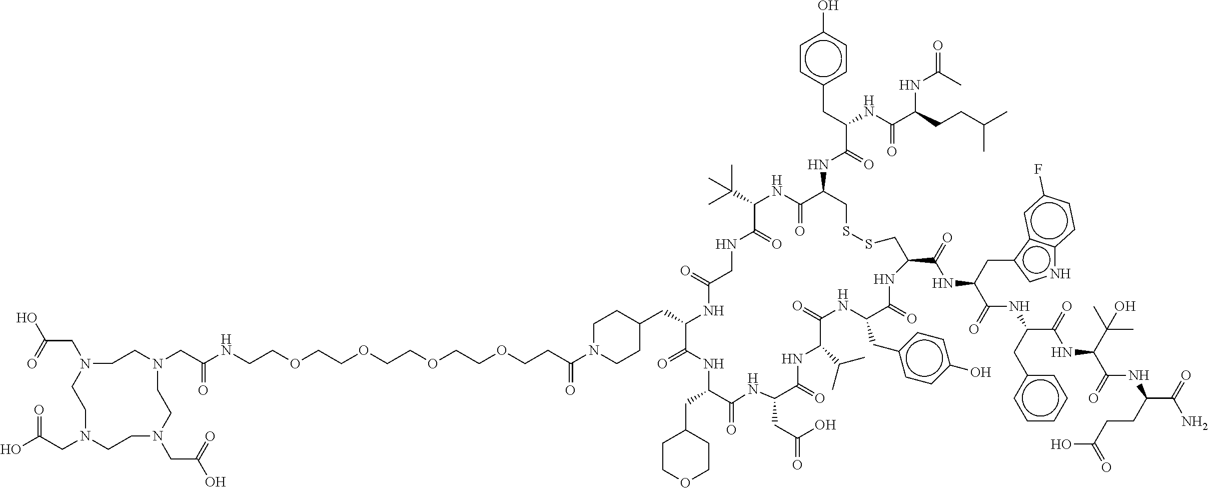 |
| 410 | 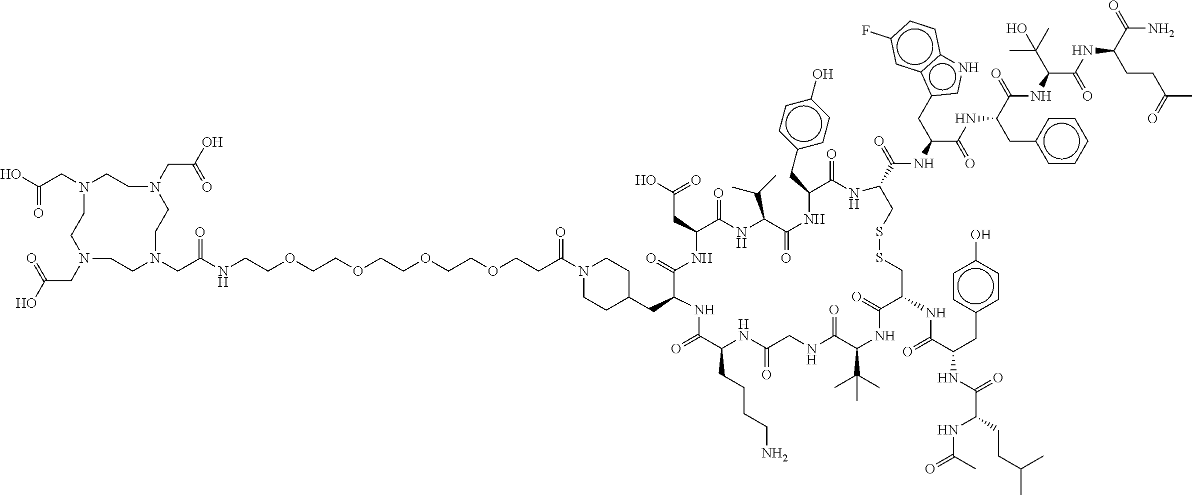 |
| 419 | 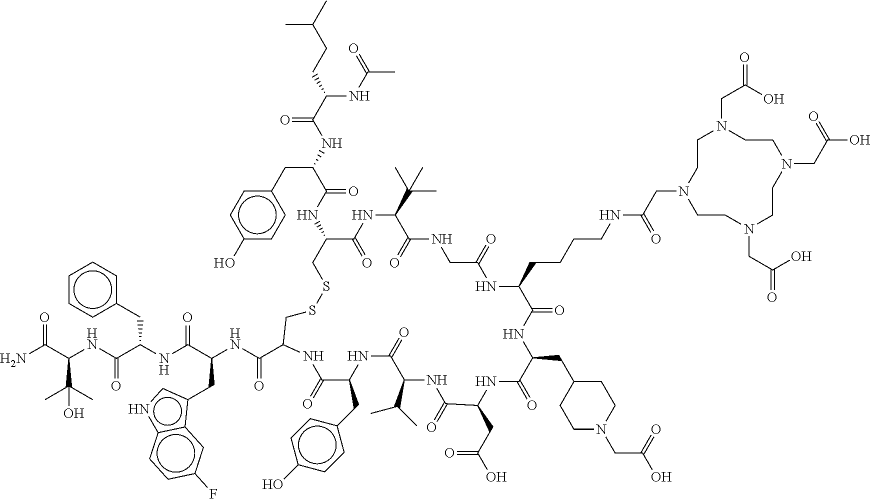 |
| 420 | 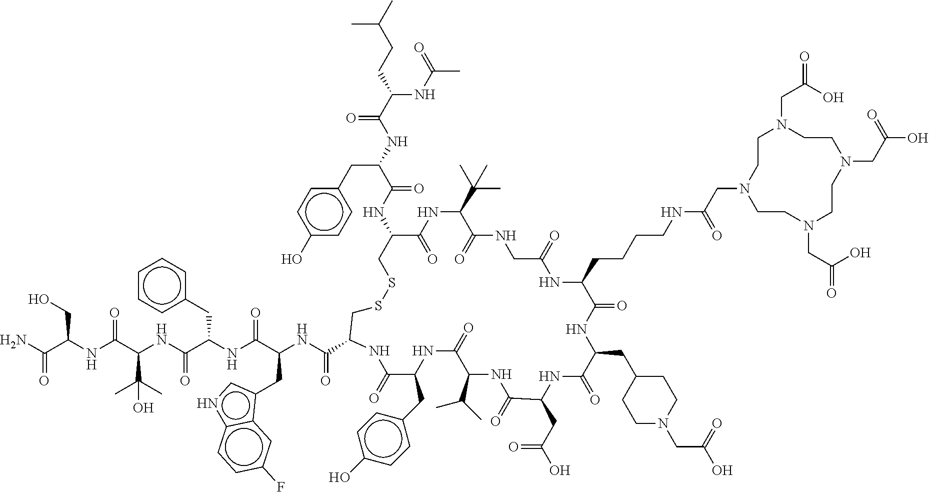 |
| 421 | 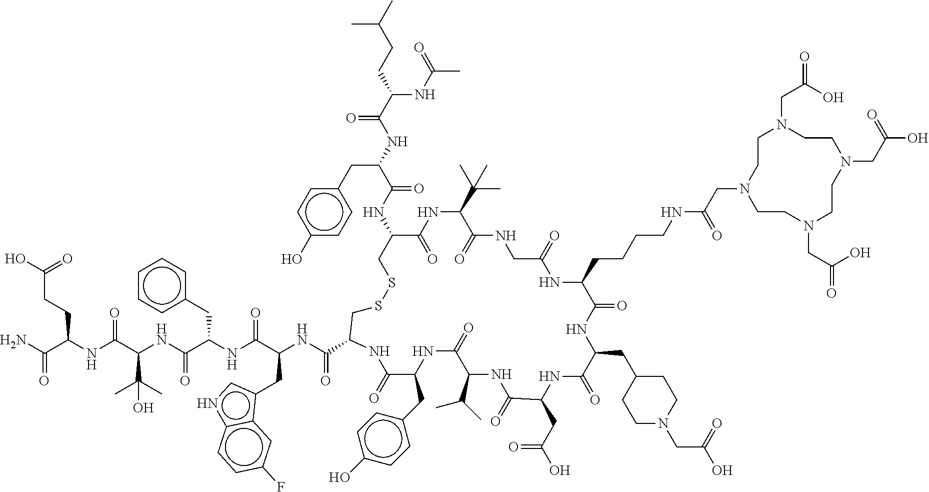 |
| 422 | 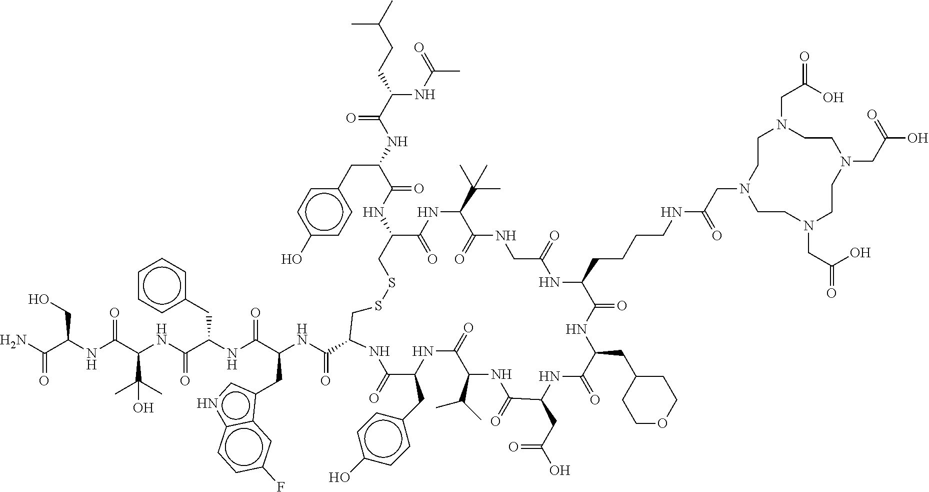 |
| 423 | 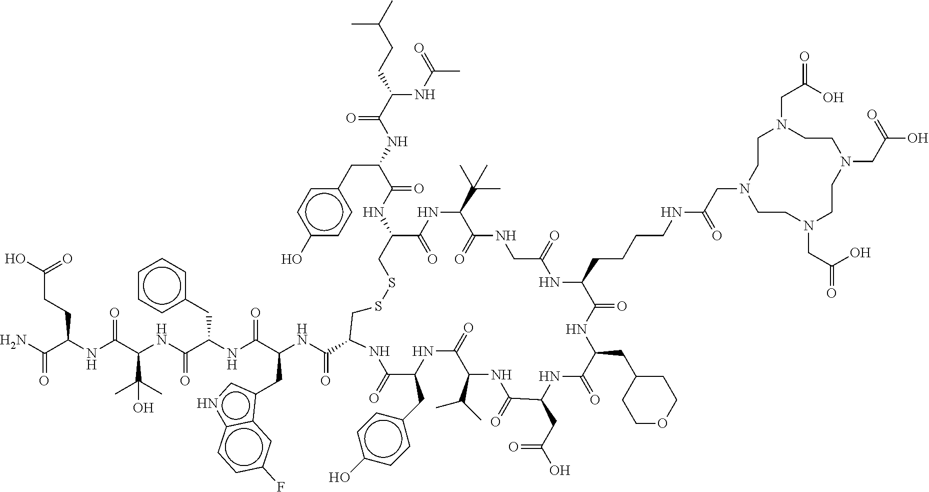 |
| 424 | 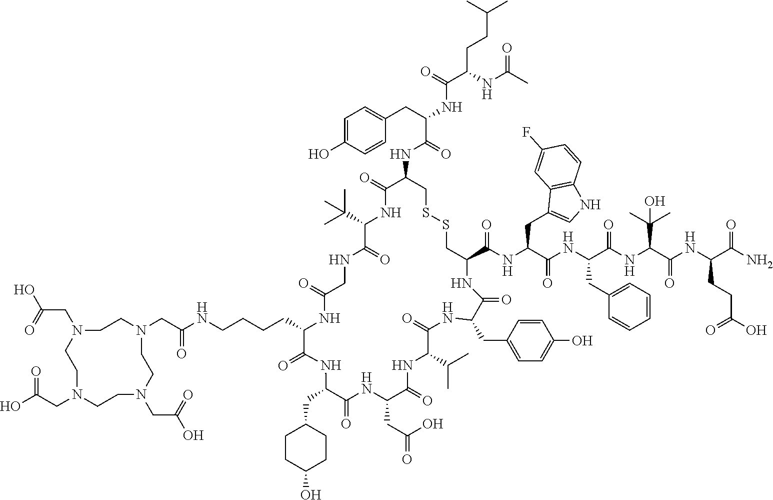 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@@H](Cc2ccccc2)C(=O)N[C@H](C(=O)N[C@H](CCC(=O)O)C(N)=O)C(C)(C)O)NC(=O)[C@H](Cc2ccc(O)cc2)NC(=O)[C@H](C(C)C)NC(=O)[C@H](CC(=O)O)NC(=O)[C@H](C[C@H]2CC[C@@H](O)CC2)NC(=O)[C@H](CCCCNC(=O)CN2CCN(CC(=O)O)CCN(CC(=O)O)CCN(CC(=O)O)CC2)NC(=O)CNC(=O)[C@H](C(C)(C)C)NC1=O] |
| 425 | 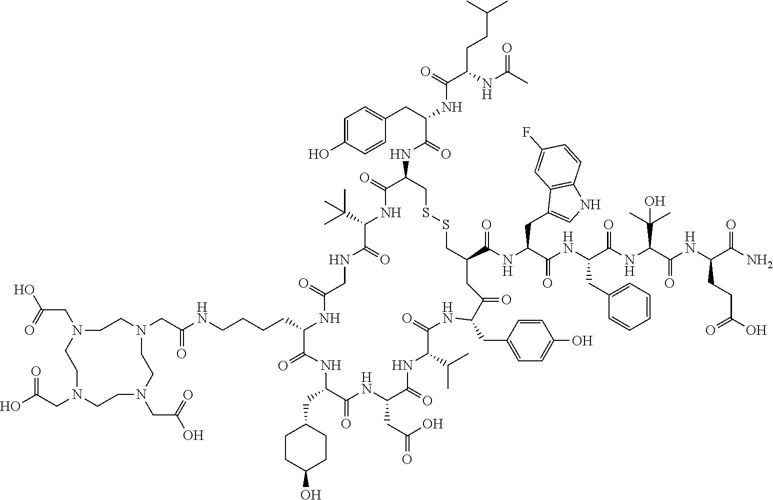 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@@H](Cc2ccccc2)C(=O)N[C@H](C(=O)N[C@H](CCC(=O)O)C(N)=O)C(C)(C)O)CC(=O)[C@H](Cc2ccc(O)cc2)NC(=O)[C@H](C(C)C)NC(=O)[C@H](CC(=O)O)NC(=O)[C@H](C[C@H]2CC[C@H](O)CC2)NC(=O)[C@H](CCCCNC(=O)CN2CCN(CC(=O)O)CCN(CC(=O)O)CCN(CC(=O)O)CC2)NC(=O)CNC(=O)[C@H](C(C)(C)C)NC1=O] |
| 427 | 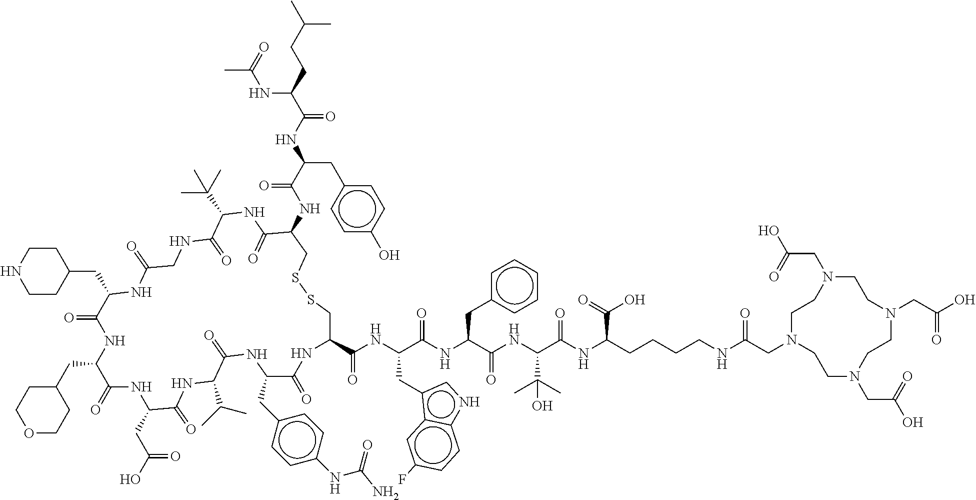 |
| 428 | 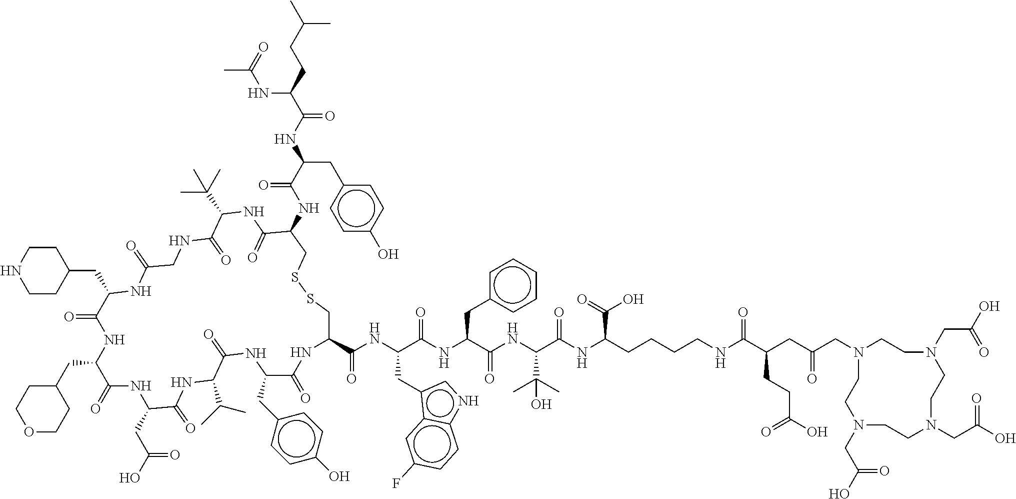 |
| 429 | 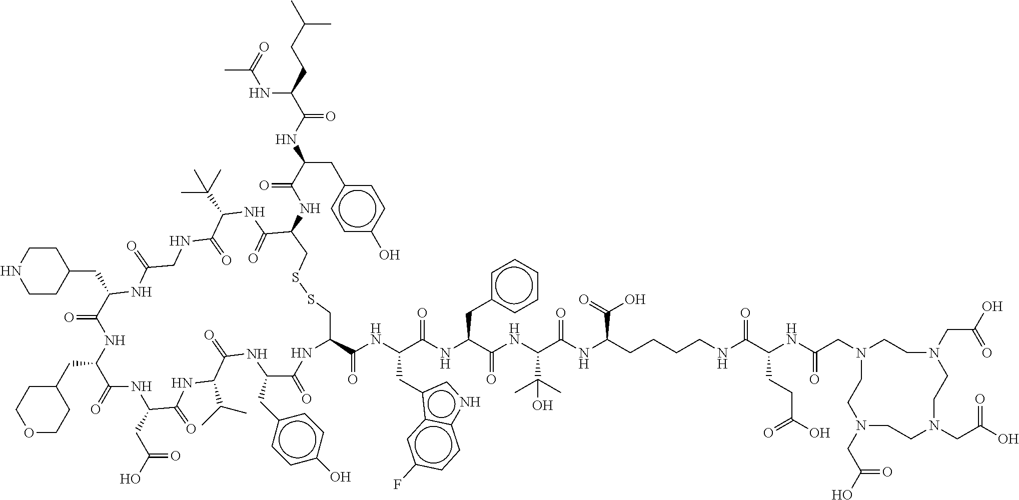 |
| 430 | 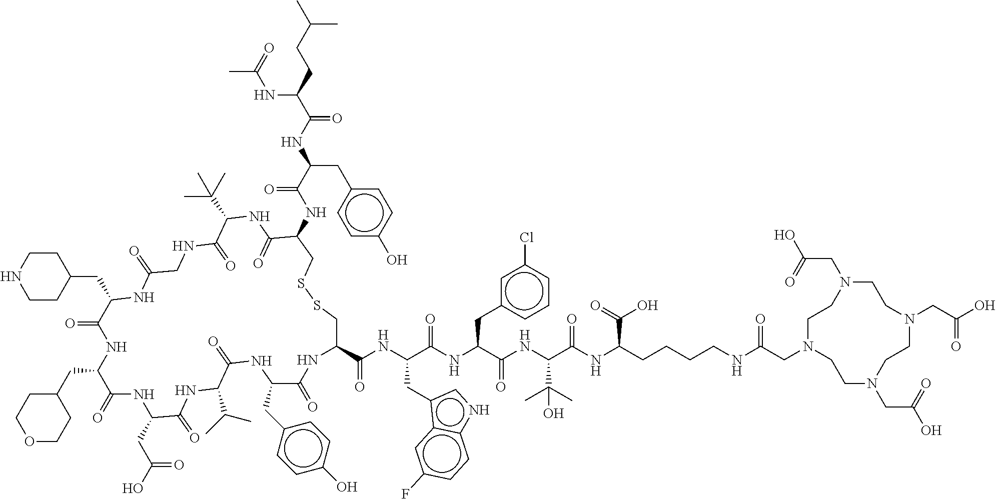 |
| 431 | 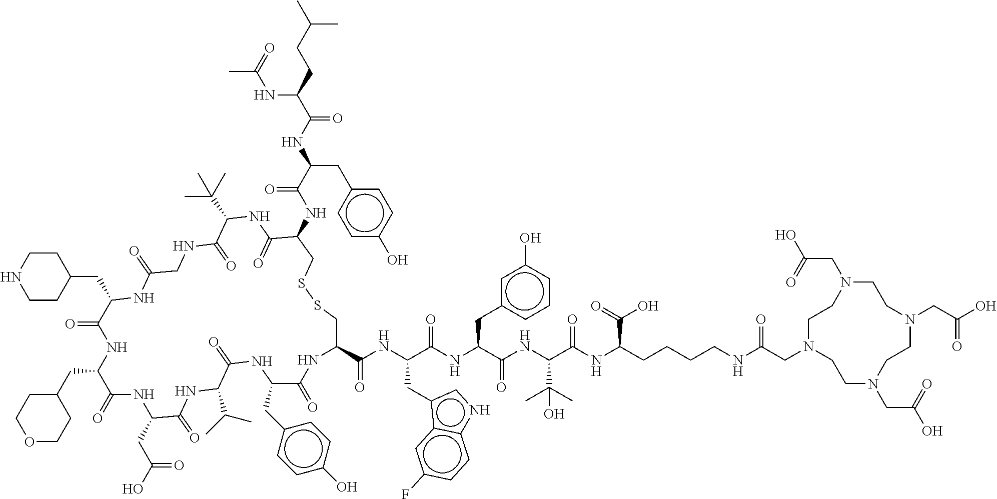 |
| 432 | 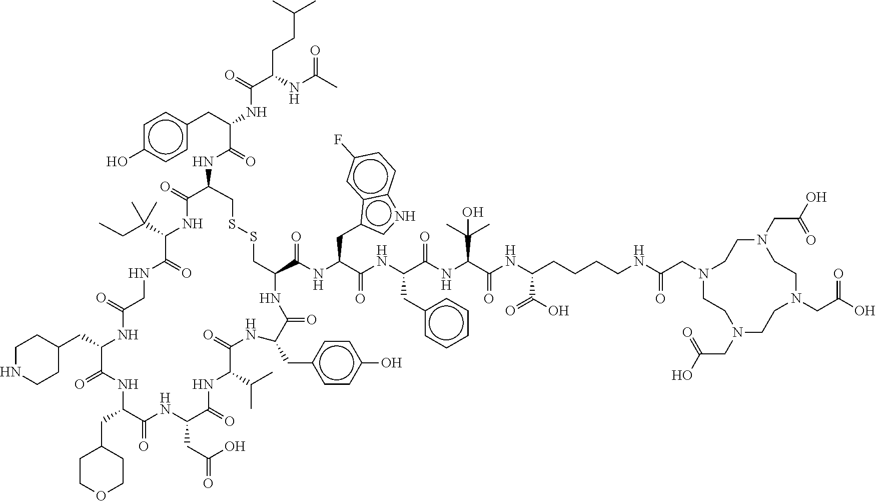 |
| 433 | 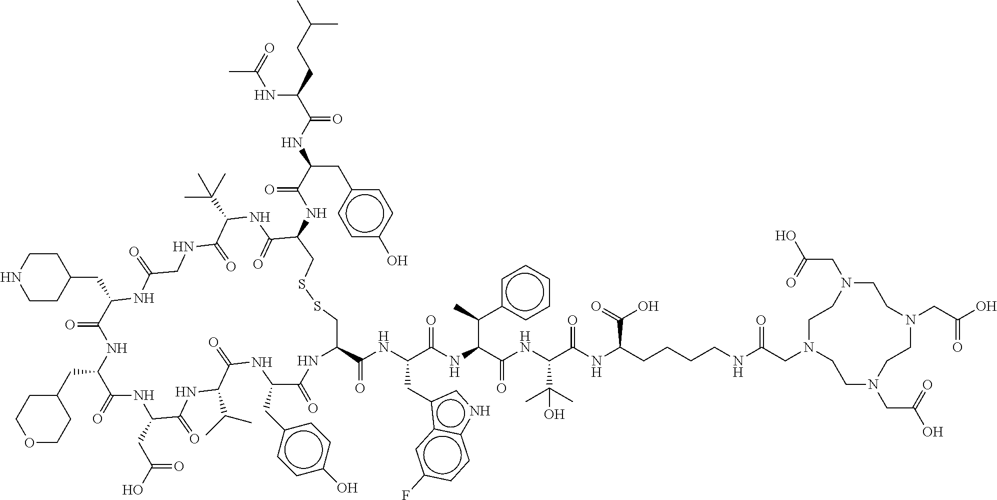 |
| 434 | 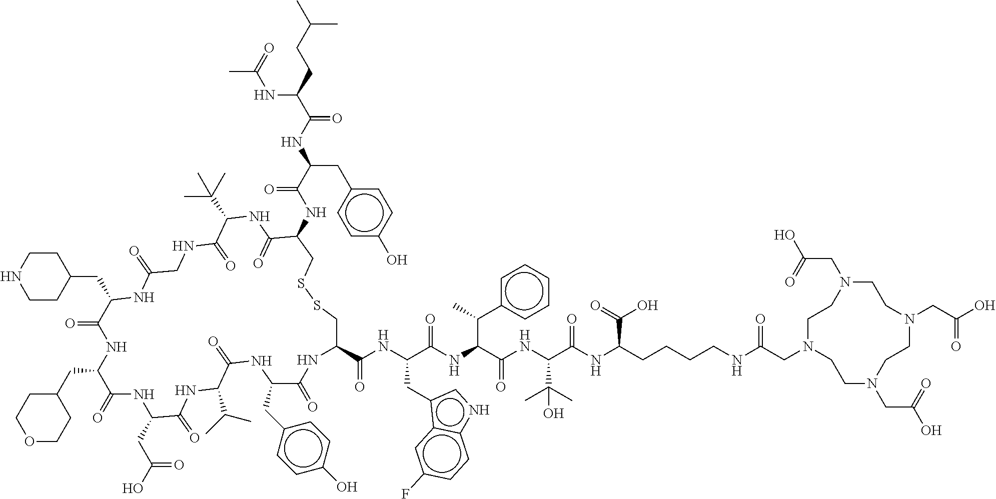 |
| 435 | 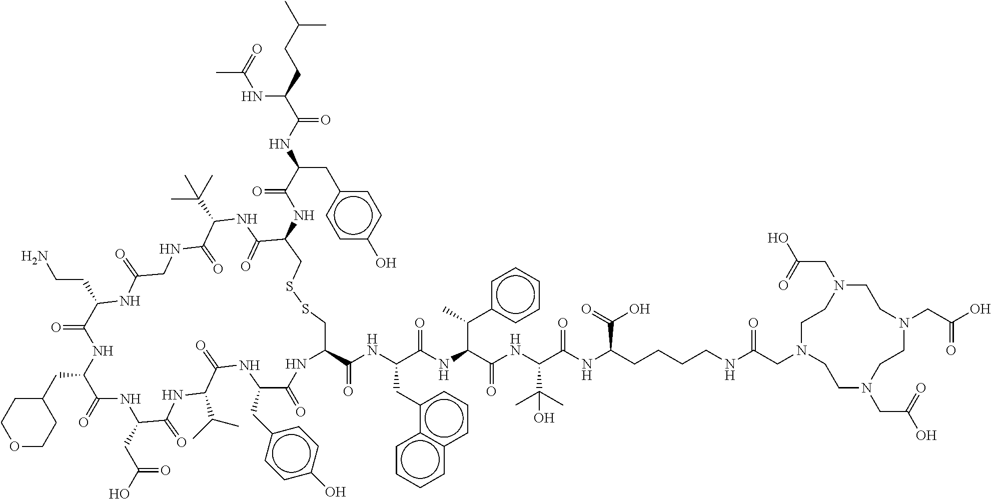 |
| 436 | 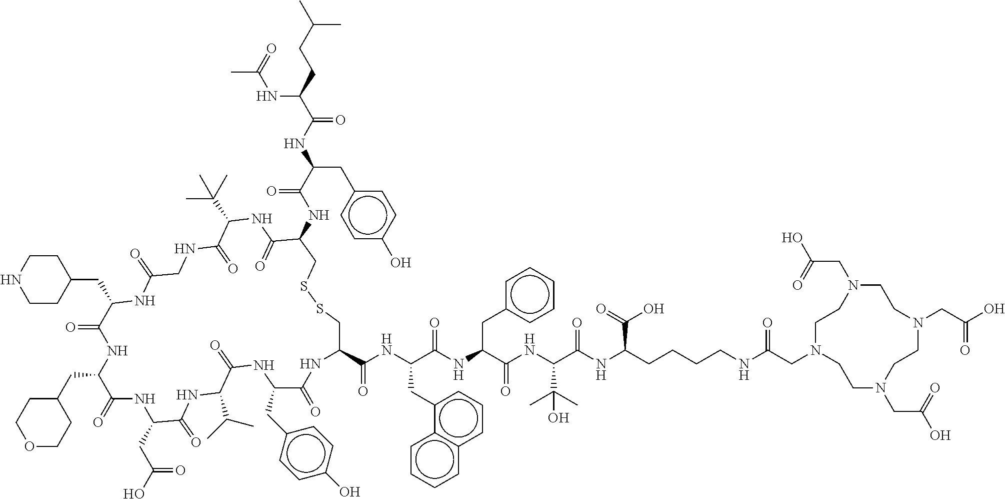 |
| 437 | 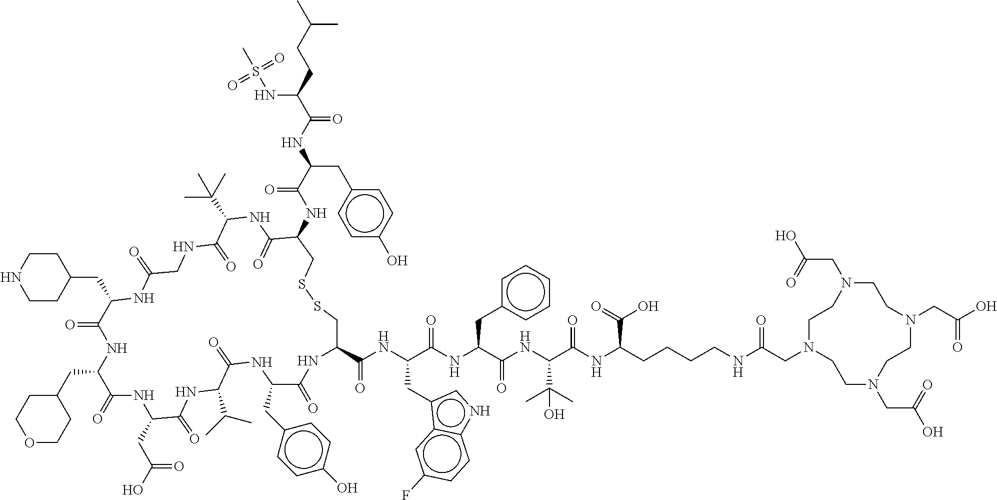 |
| 438 | 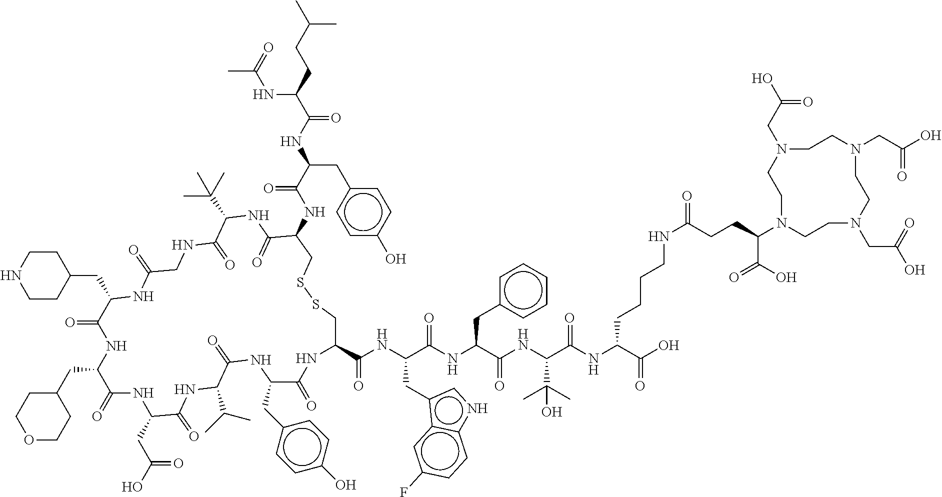 |
| 439 | 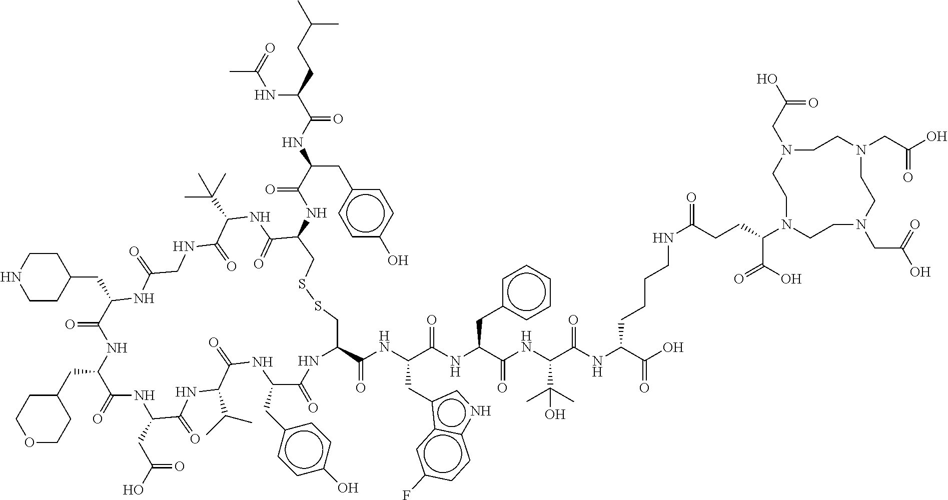 |
| 440 | 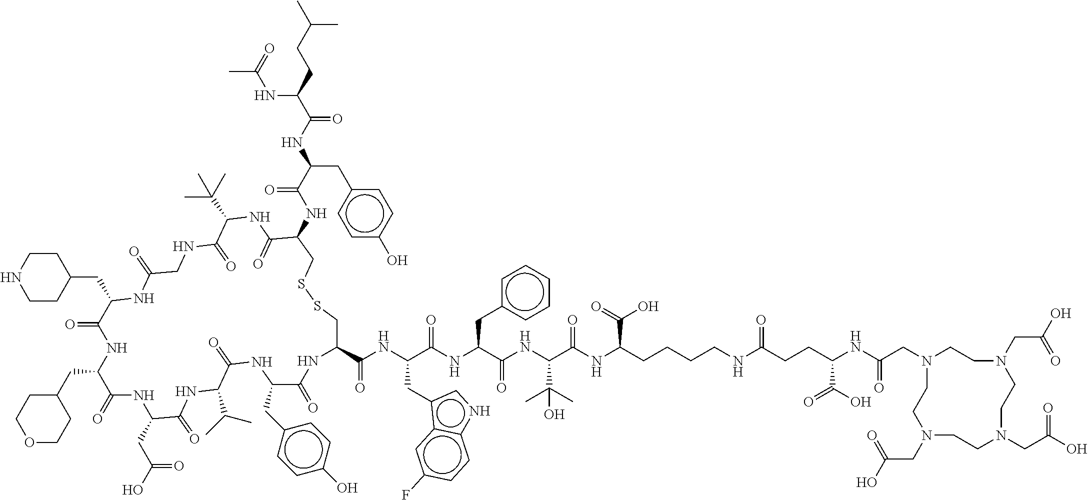 |
| 441 | 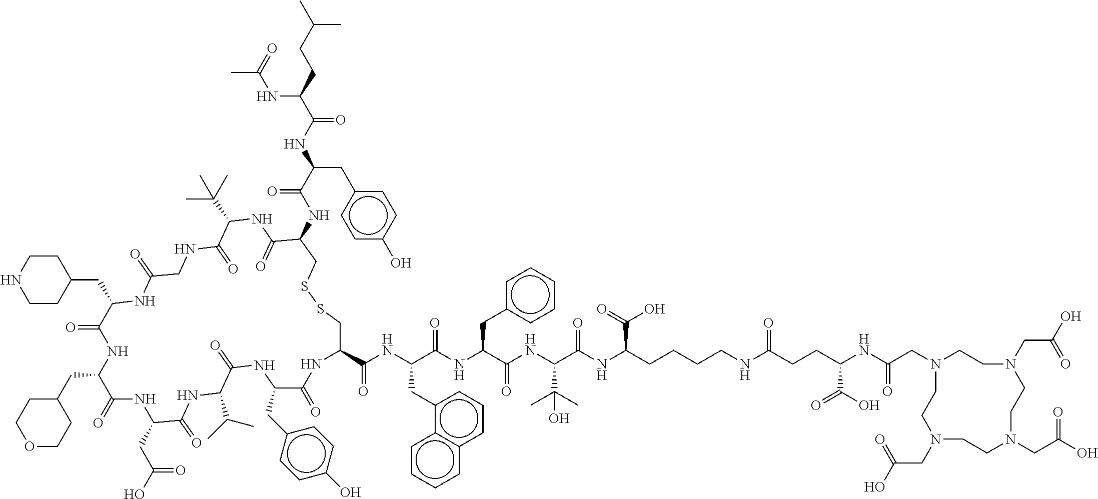 |
| 442 | 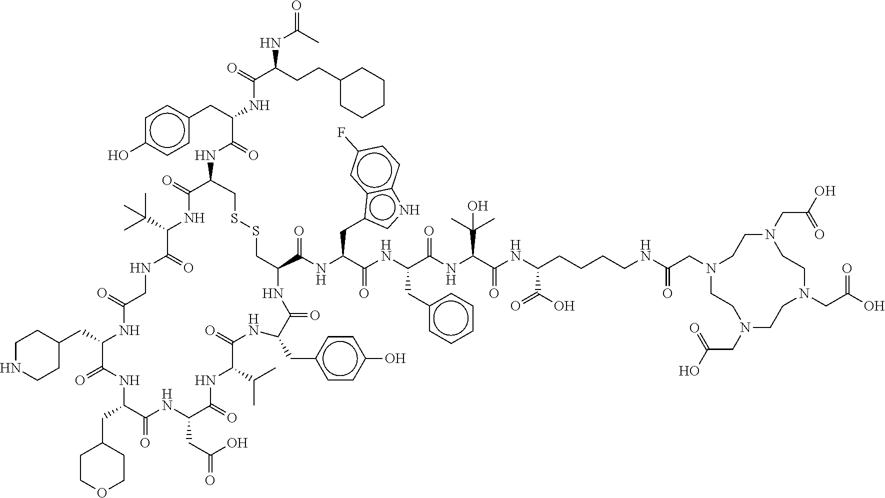 |
| 443 | 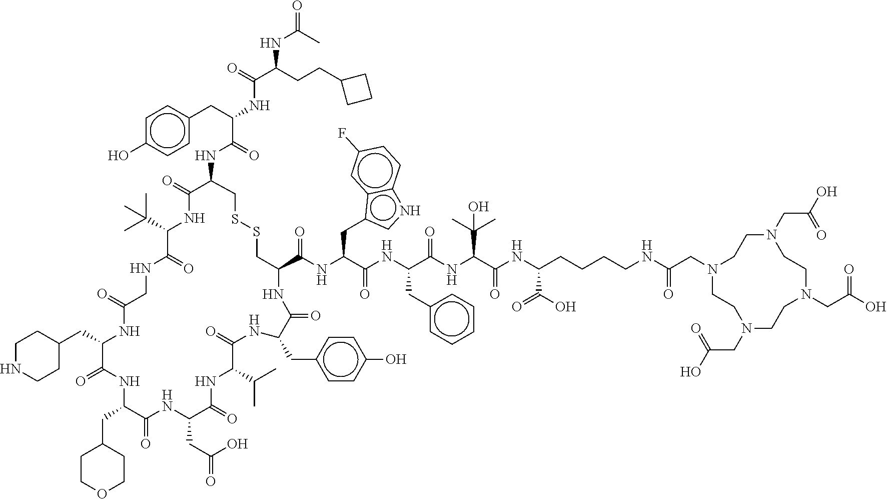 |
| 444 | 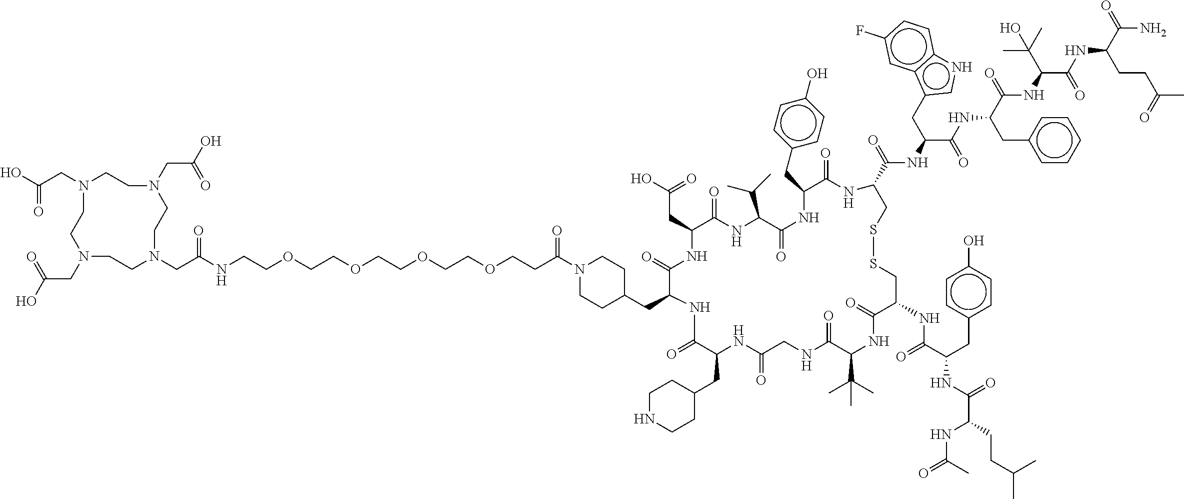 |
| 445 | 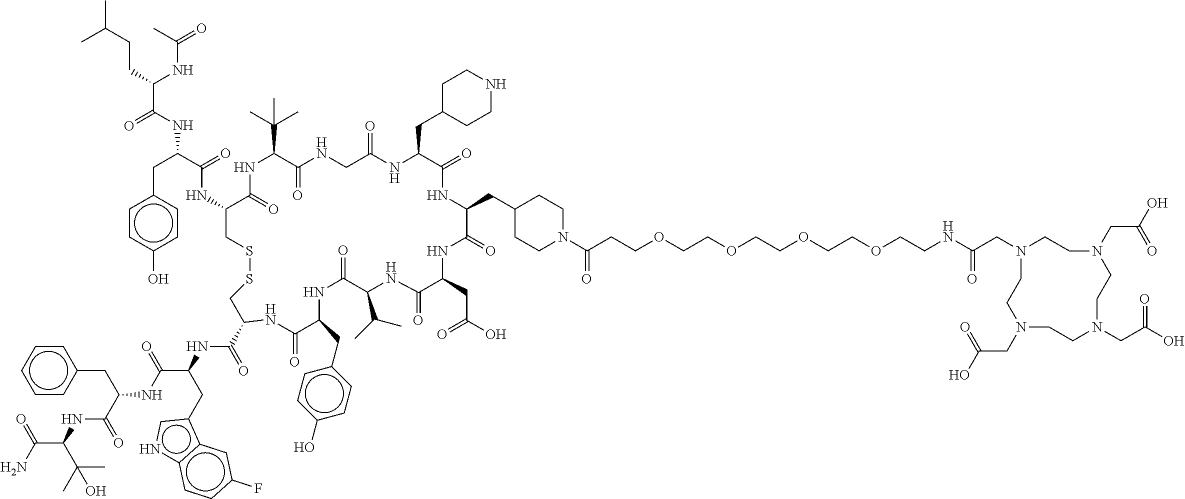 |
| 446 | 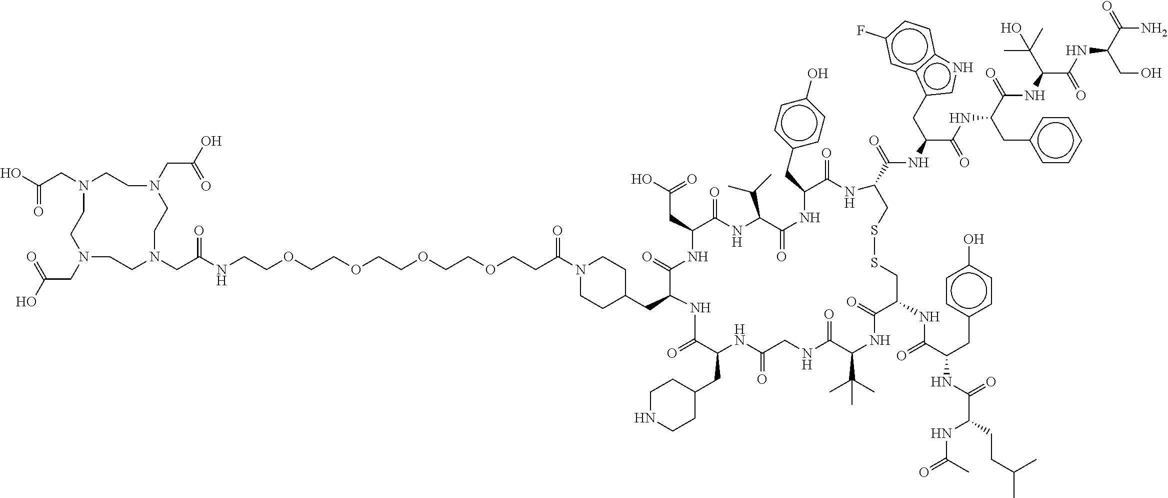 |
| 447 | 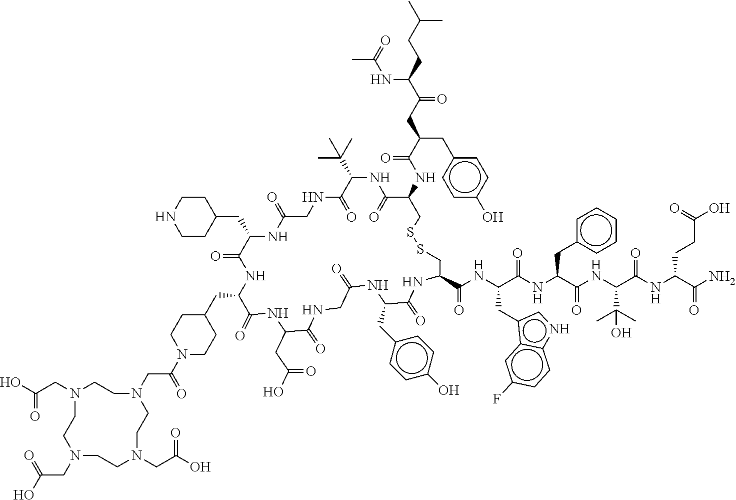 |
| 448 | 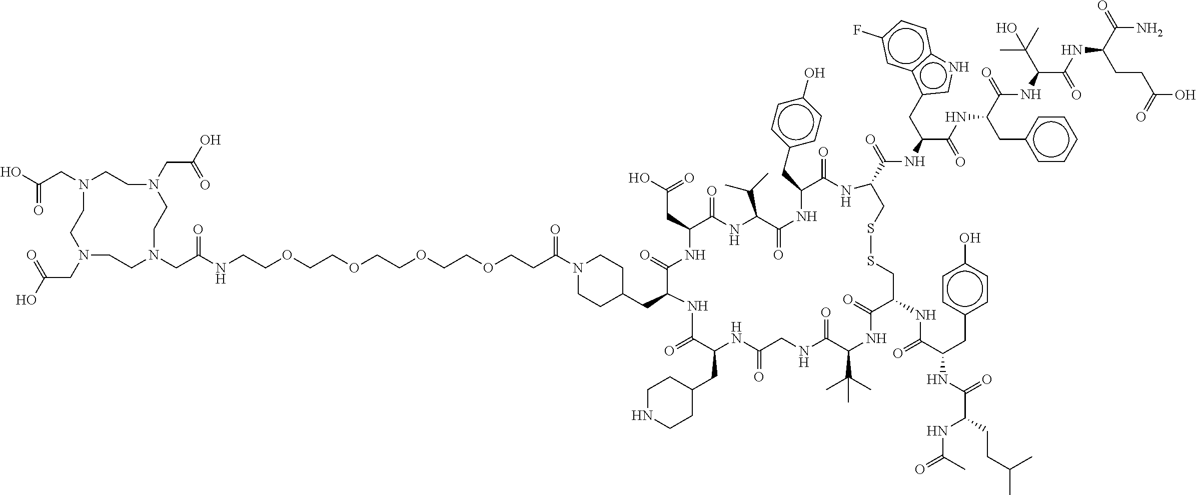 |
| 449 | 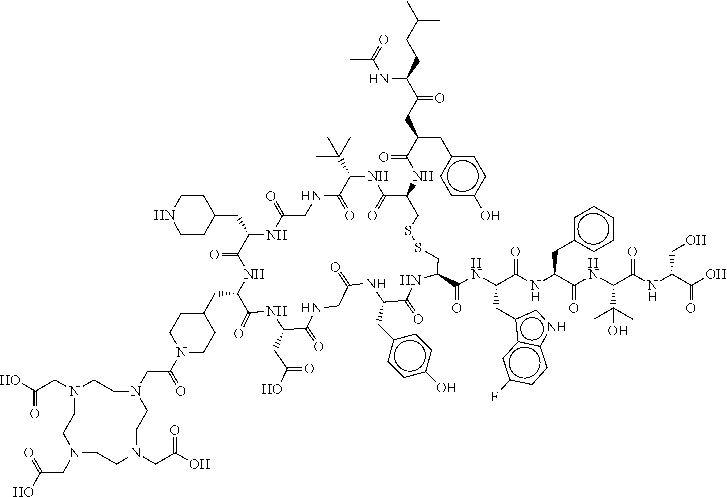 |
| 450 | 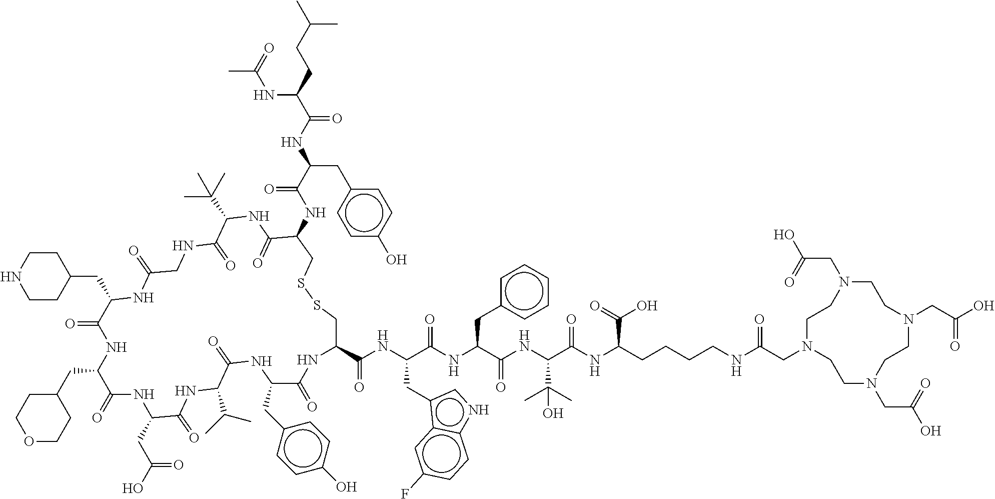 |
| 451 | 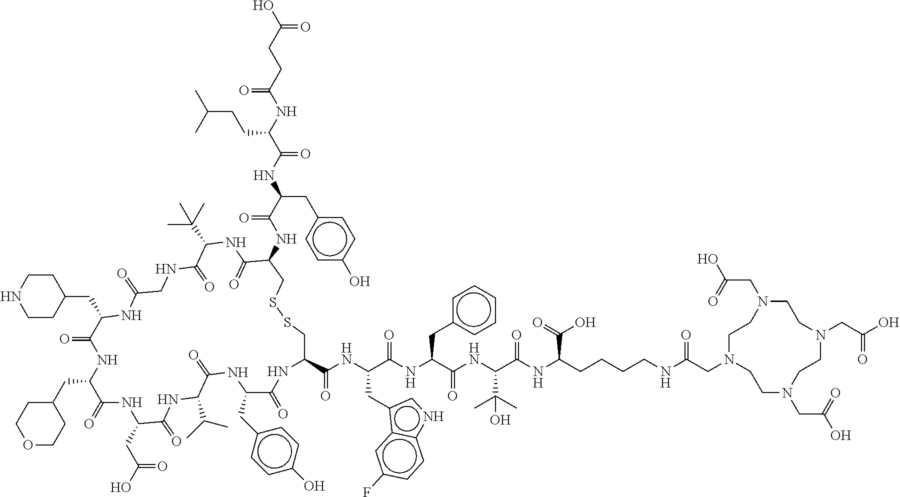 |
| 452 | 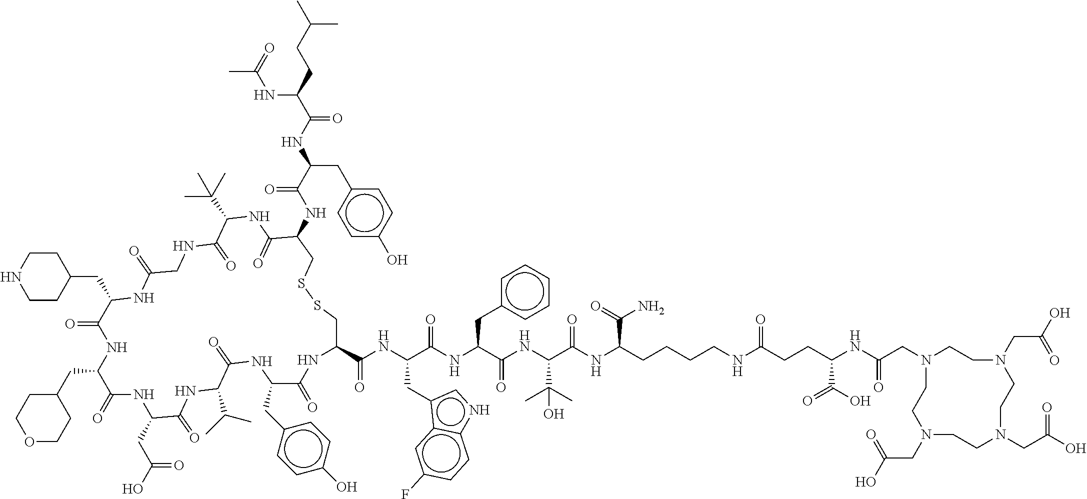 |
| 453 | 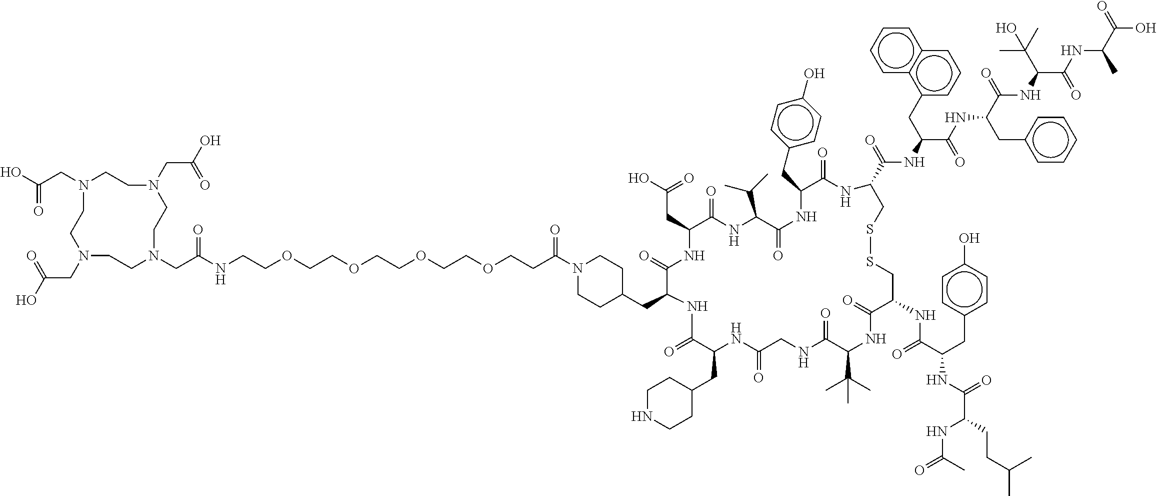 |
| 454 | 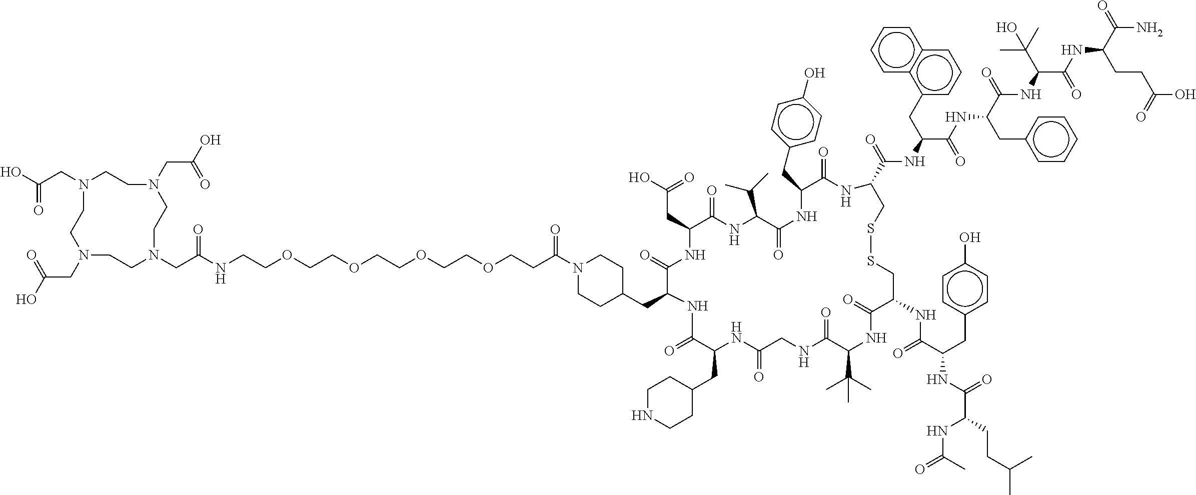 |
| 455 | 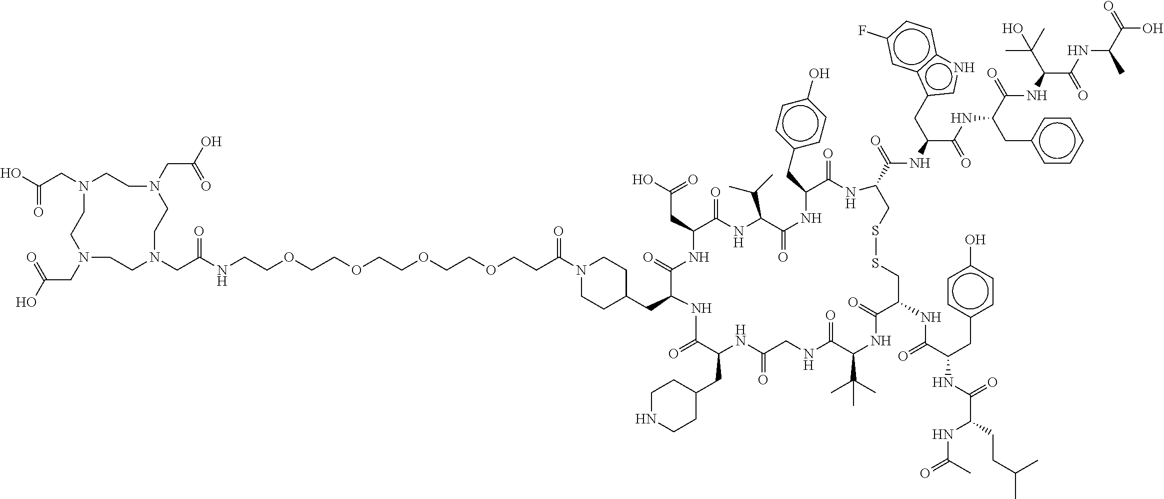 |
| 456 | 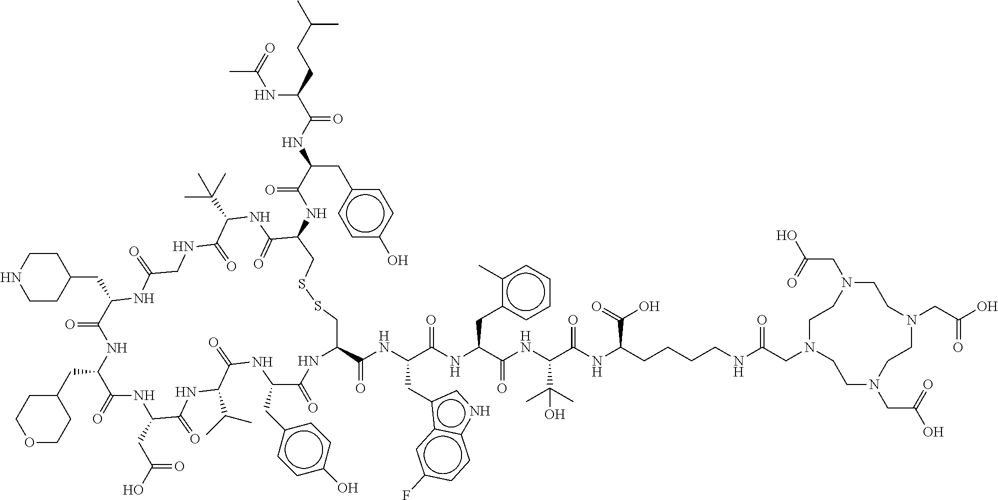 |
| 457 | 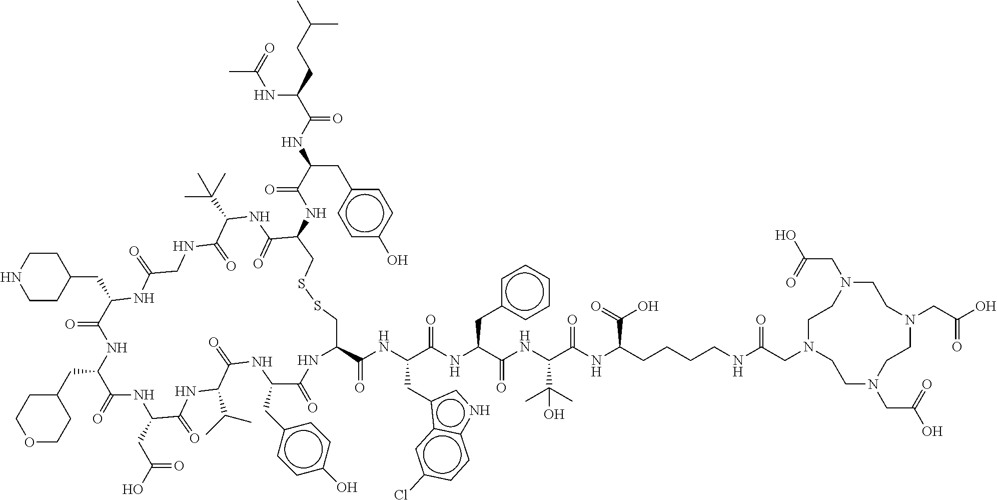 |
| 458 | 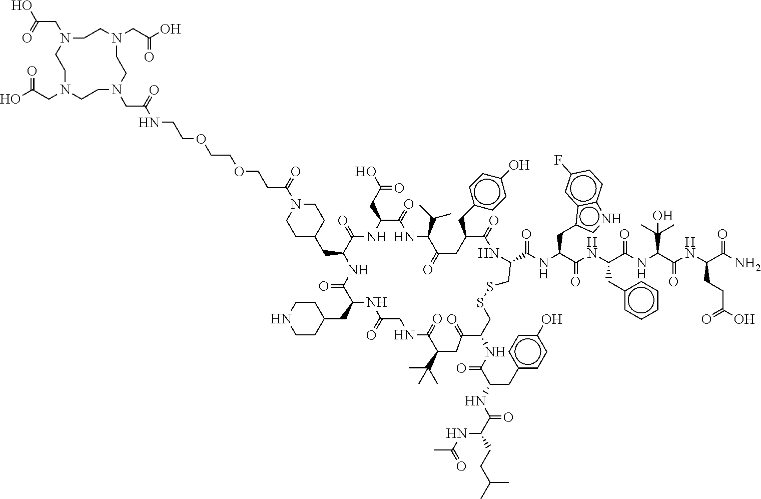 |
| 459 | 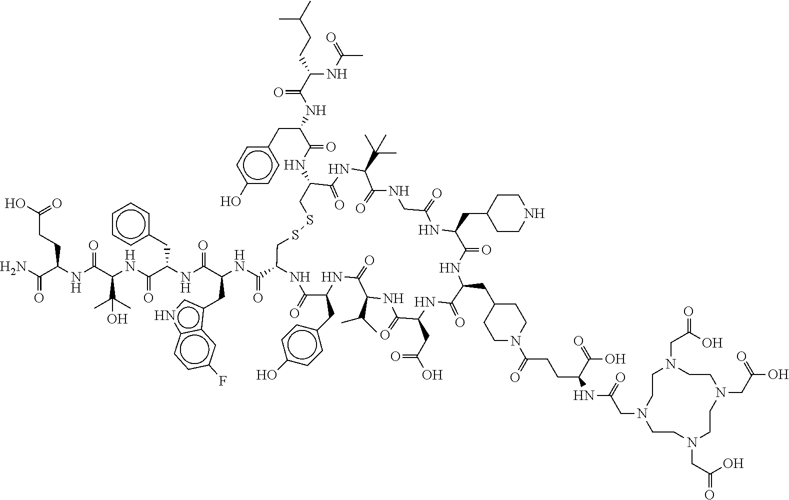 |
| 460 | 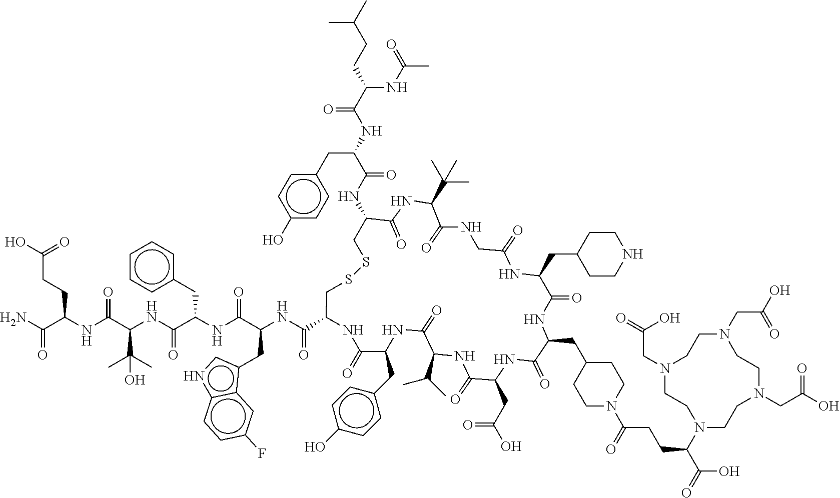 |
| 461 | 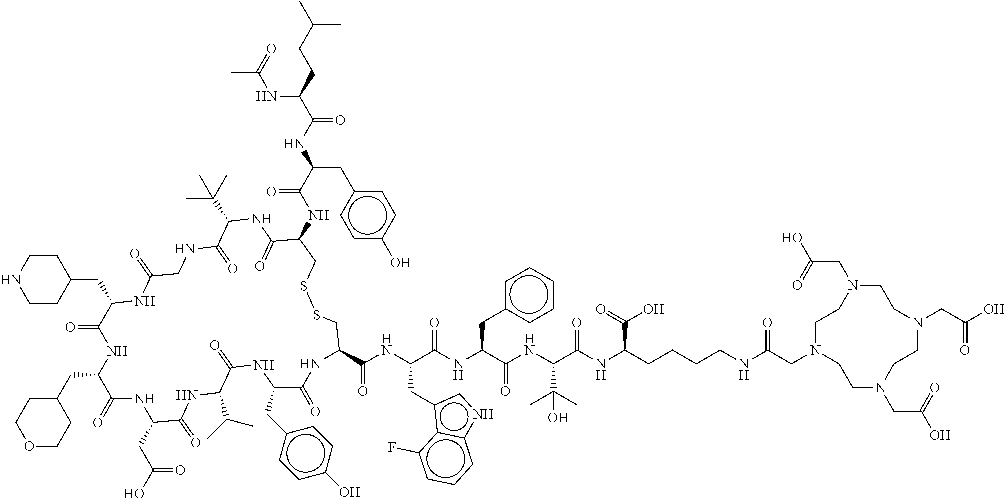 |
| 462 | 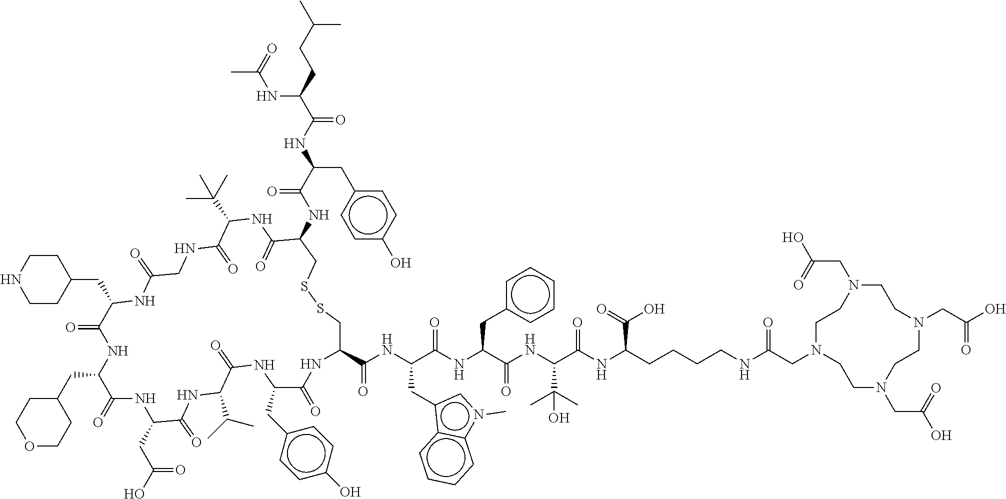 |
| 463 | 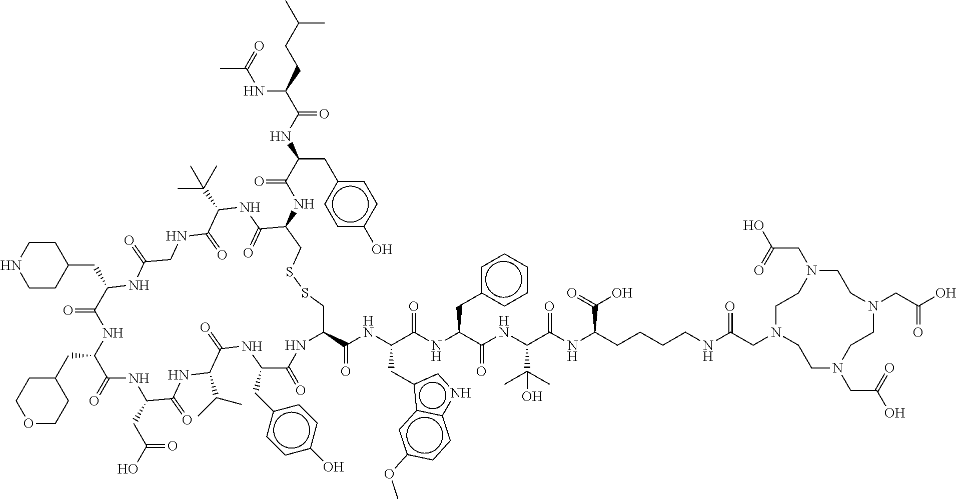 |
| 464 | 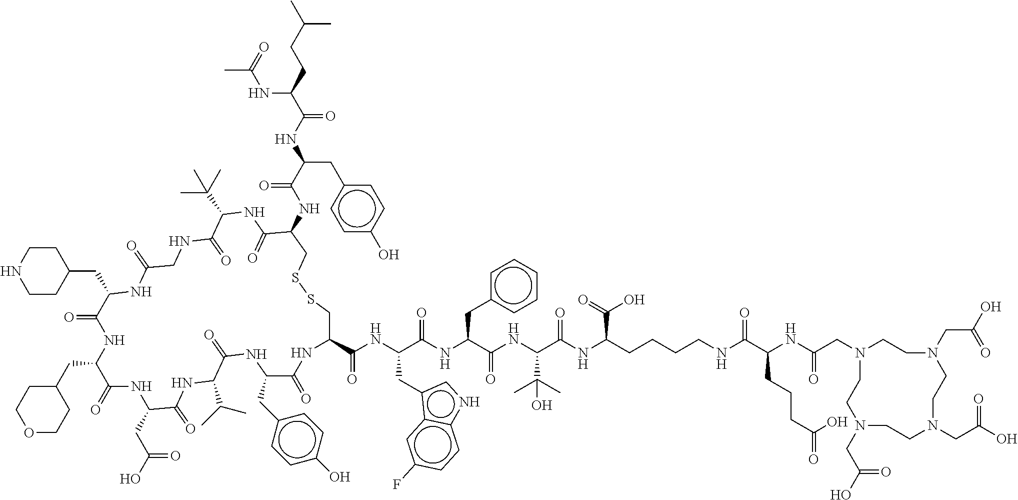 |
| 465 | 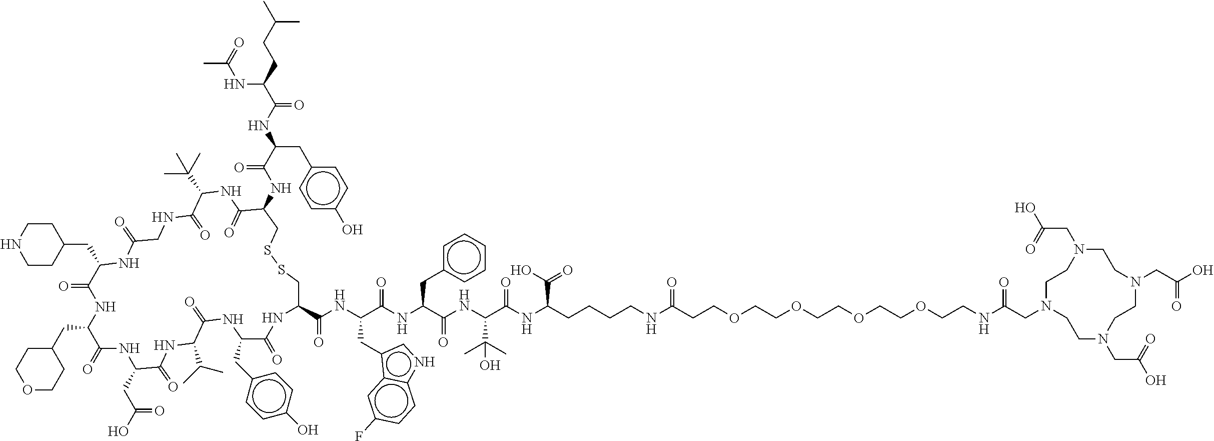 |
| 467 | 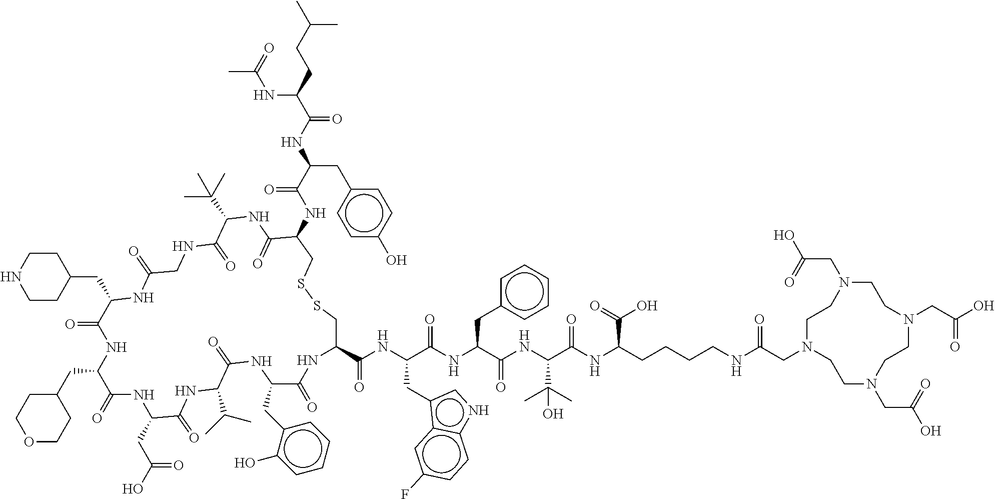 |
| 468 | 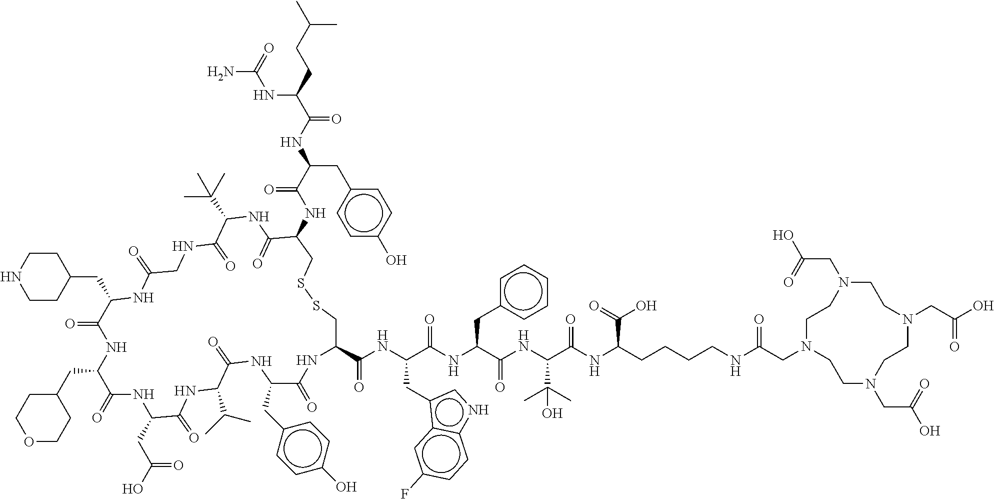 |
| 469 | 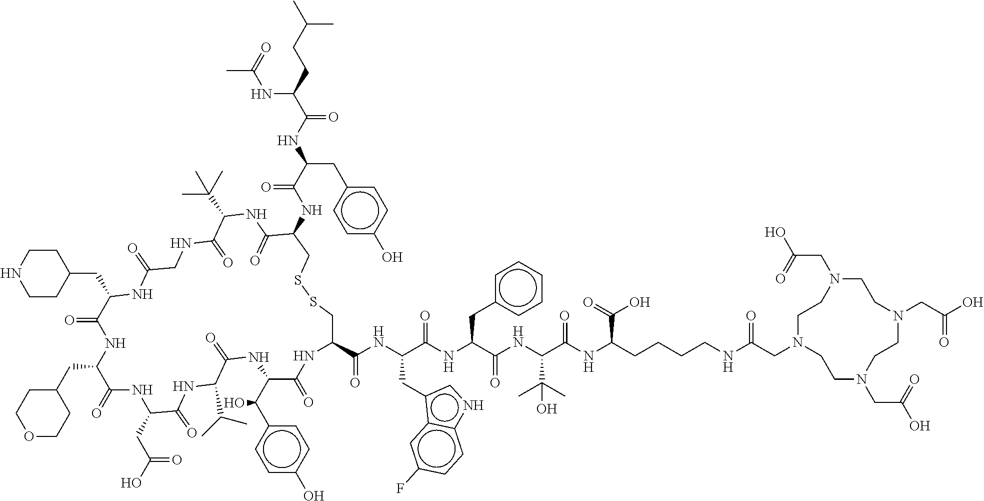 |
| 470 | 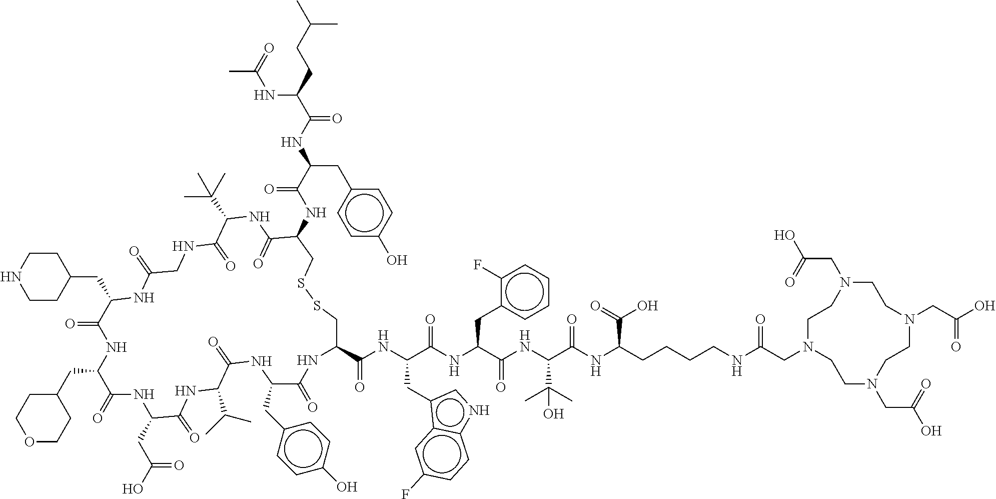 |
| 471 | 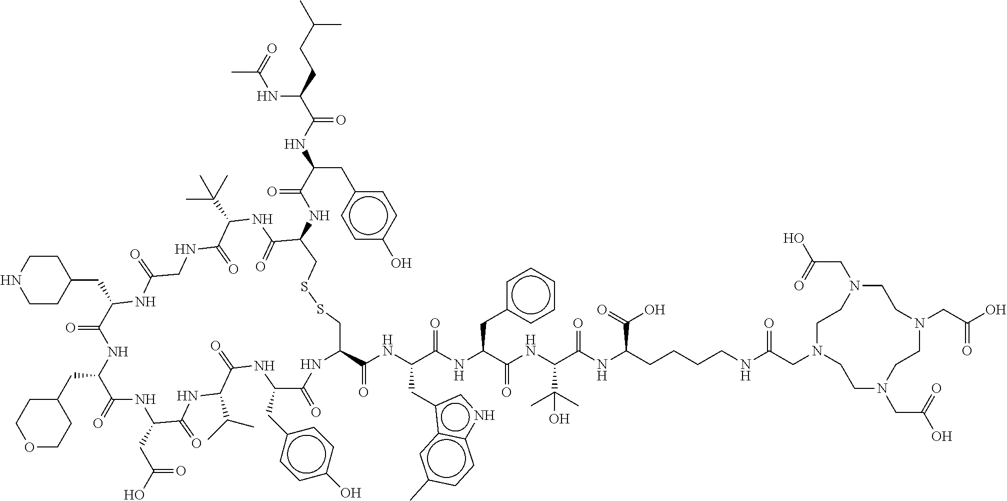 |
| 472 | 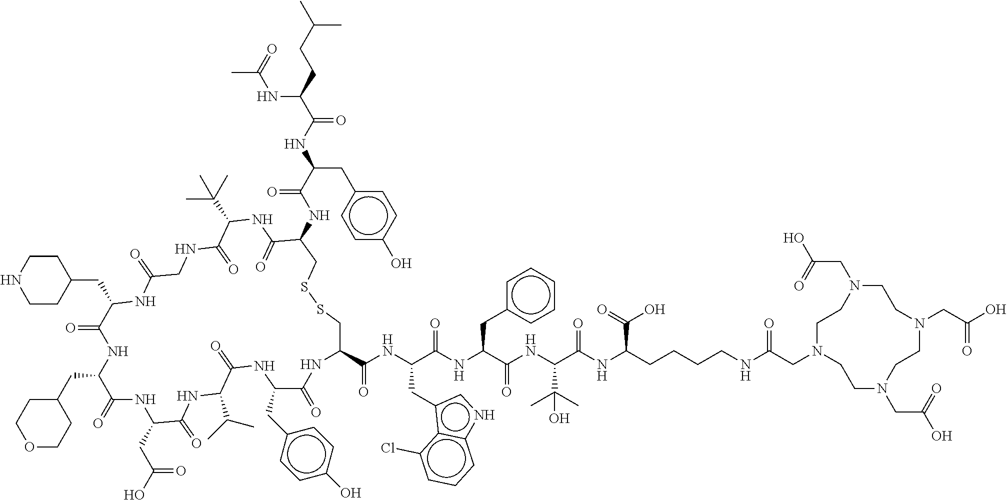 |
| 473 | 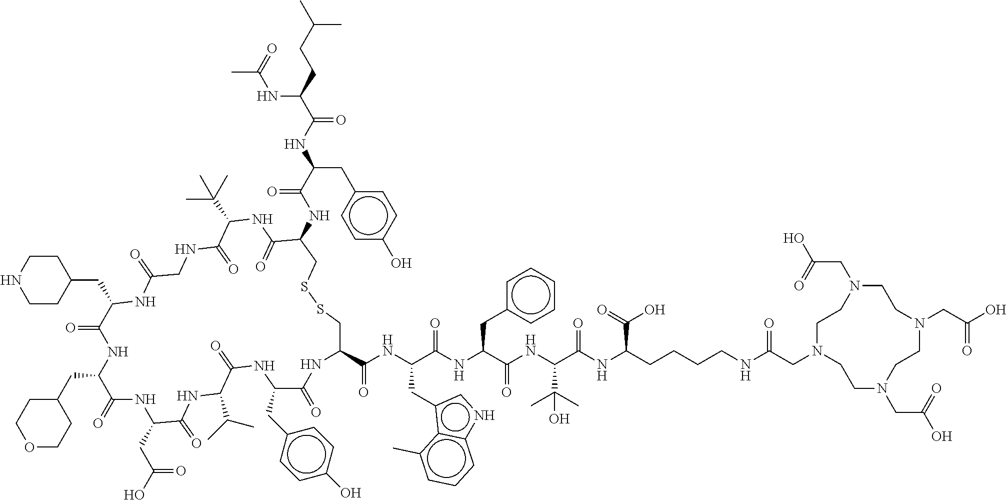 |
| 474 | 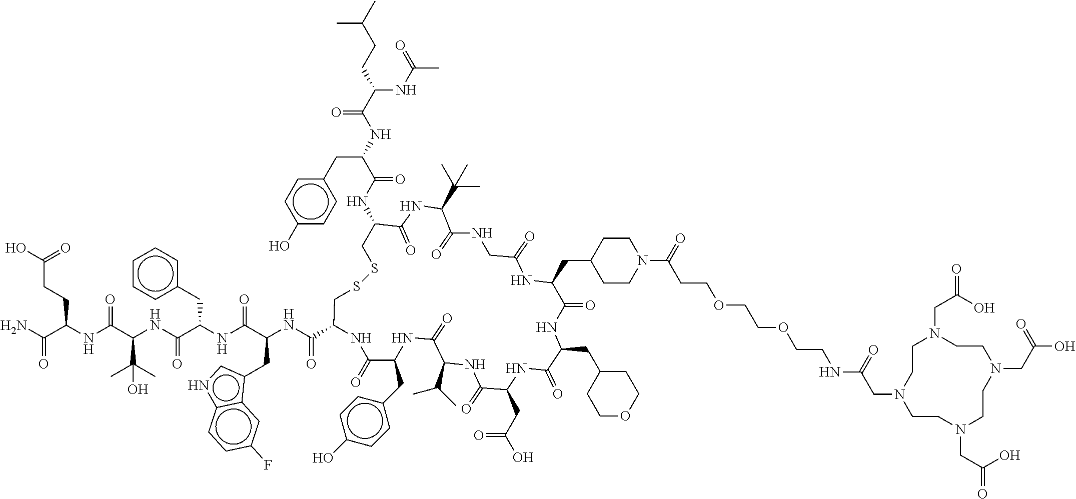 |
| 475 | 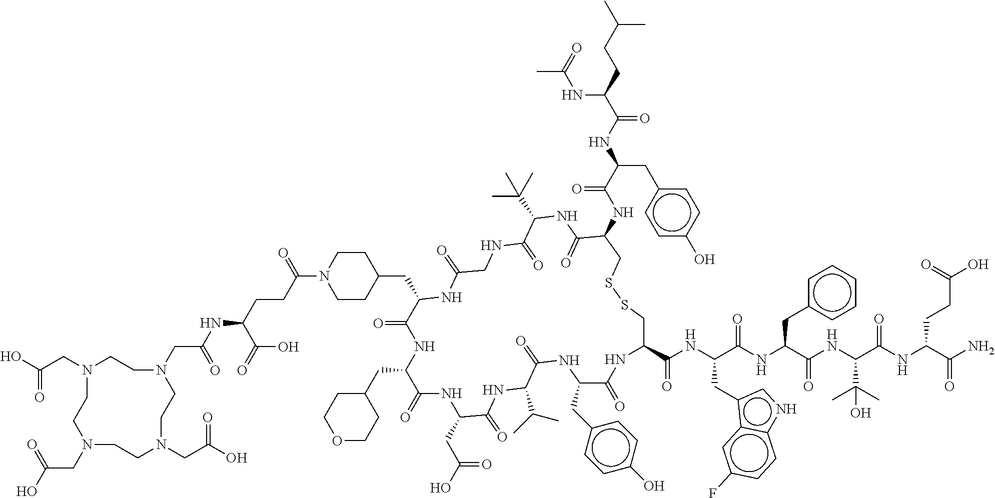 |
| 476 | 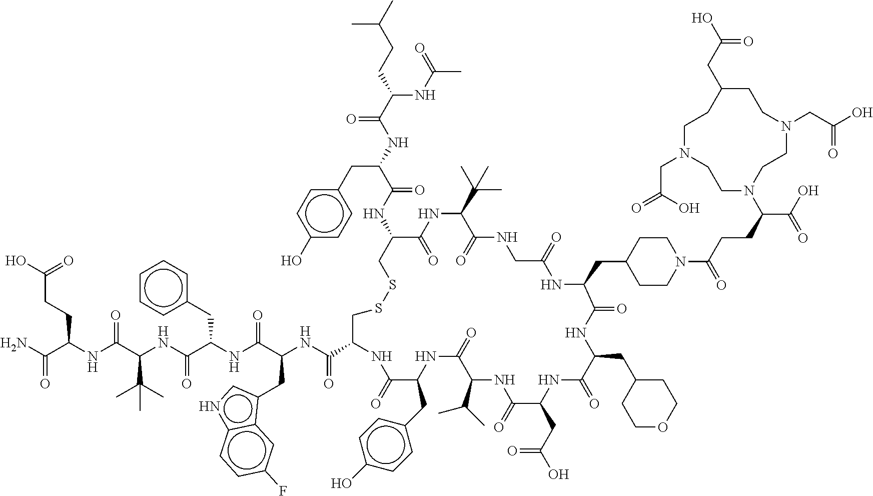 |
| 477 | 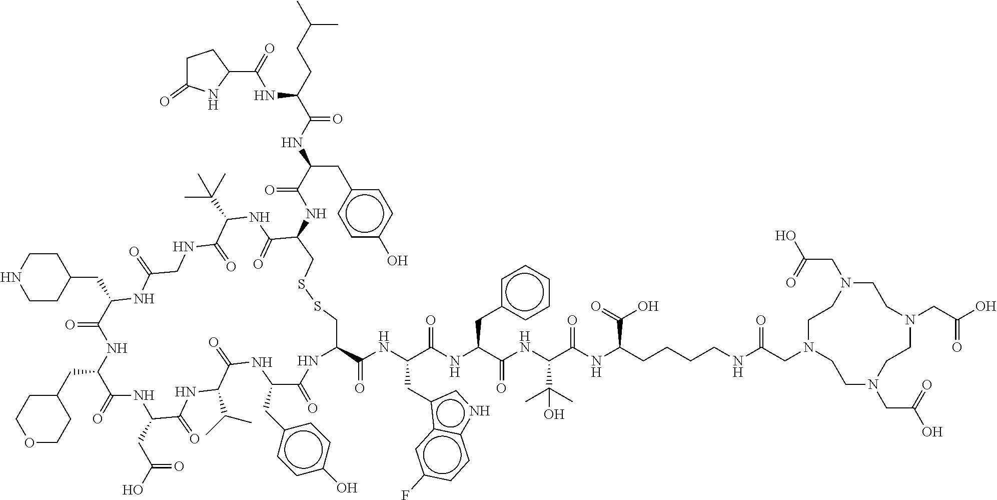 |
| 478 | 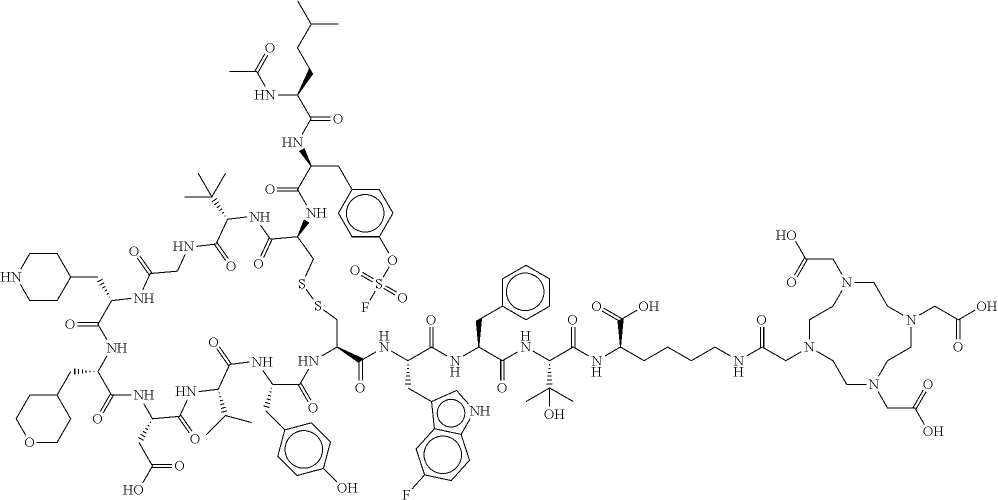 |
| 479 | 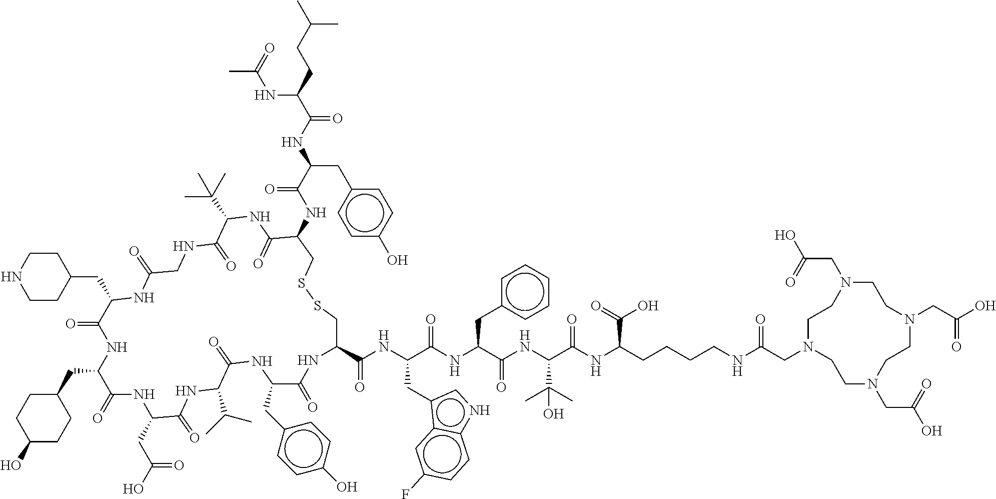 |
| 480 | 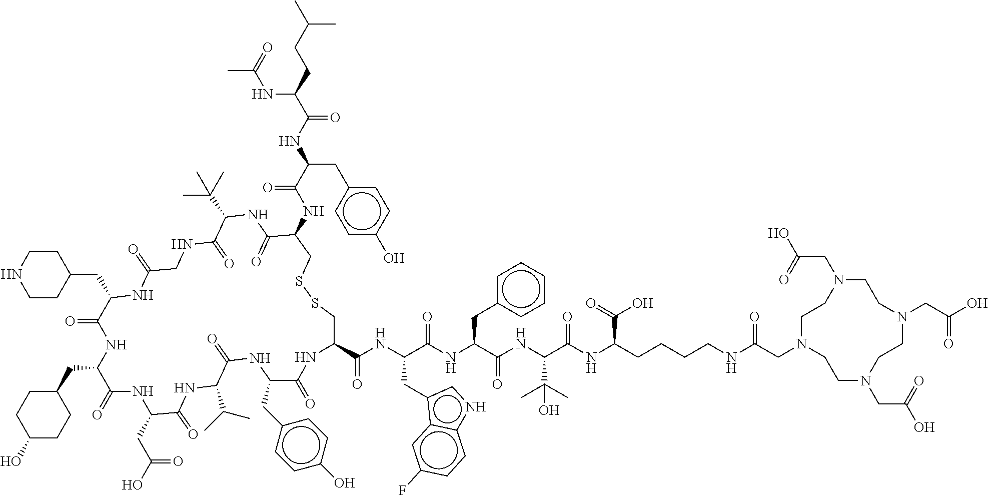 |
| 481 | 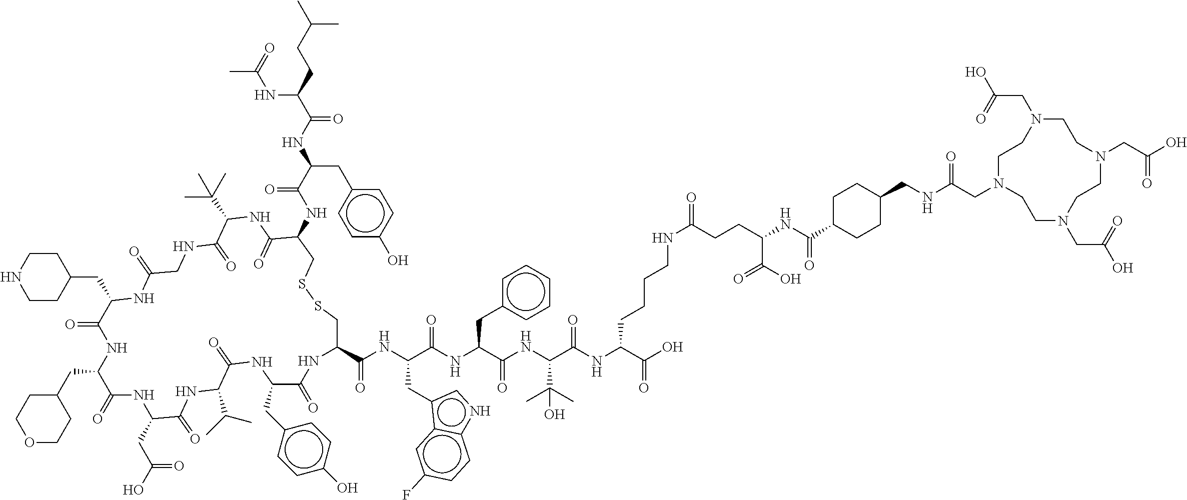 |
| 482 | 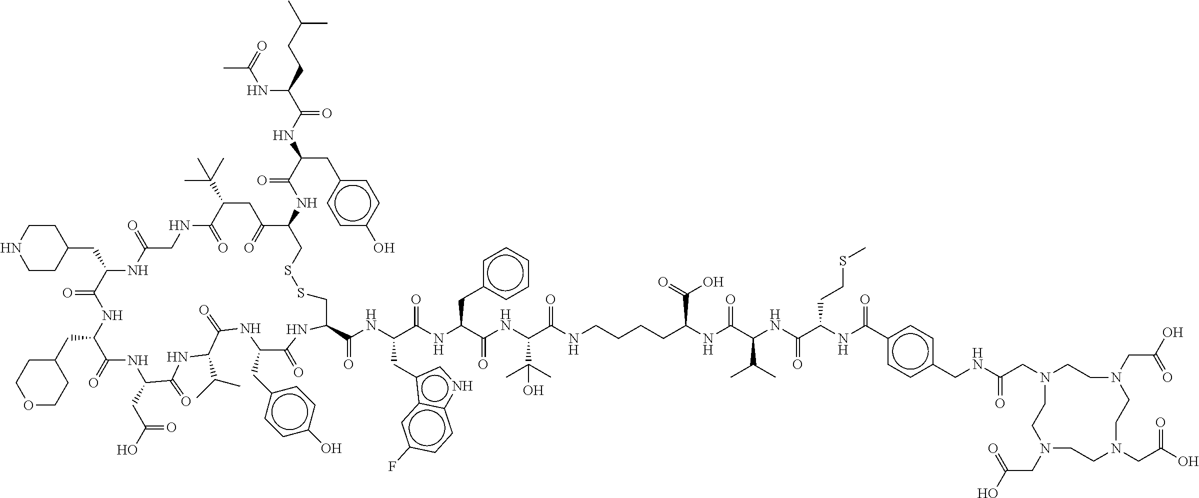 |
| 483 | 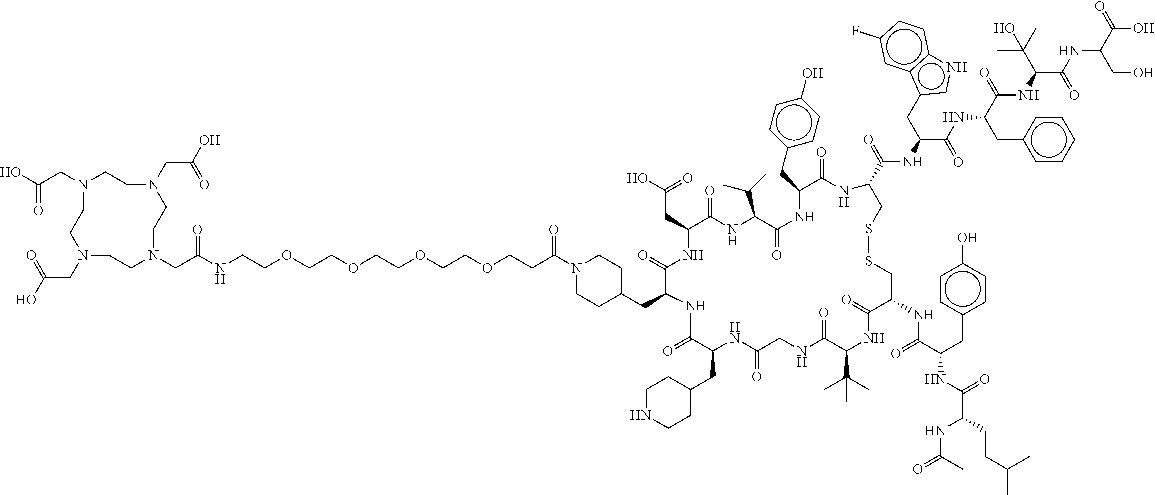 |
| 484 | 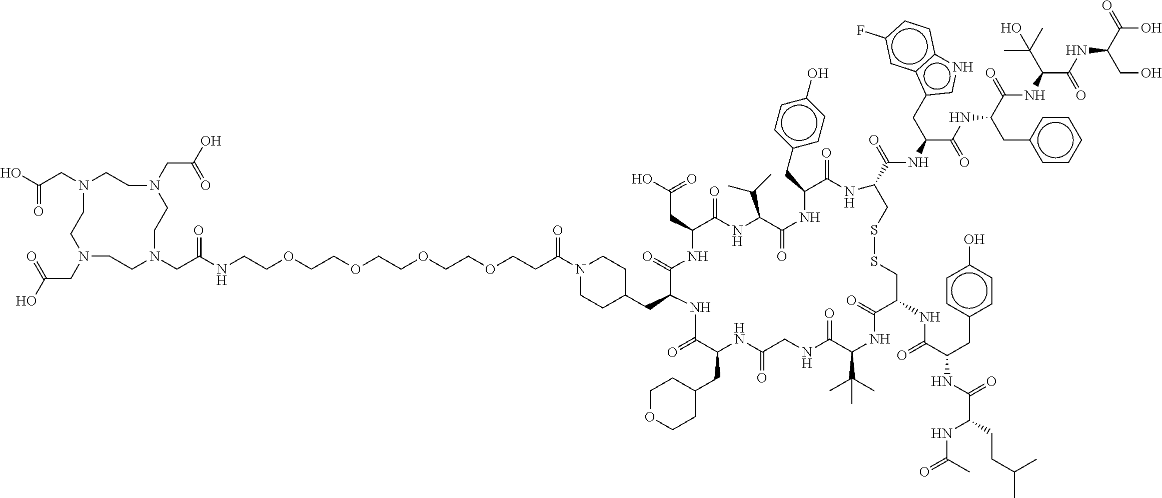 |
| 485 | 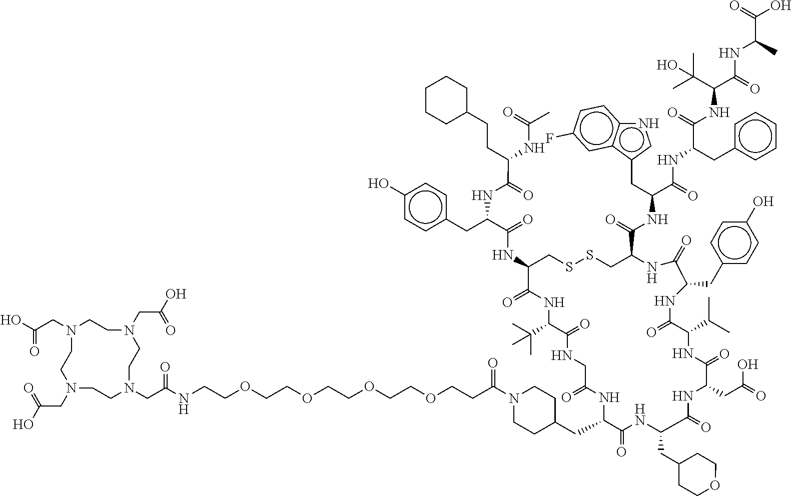 |
| 486 | 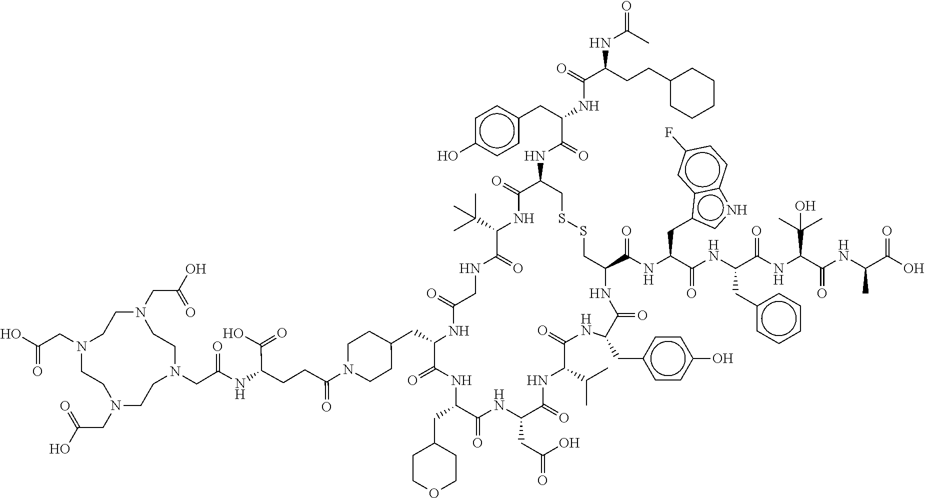 |
| 487 | 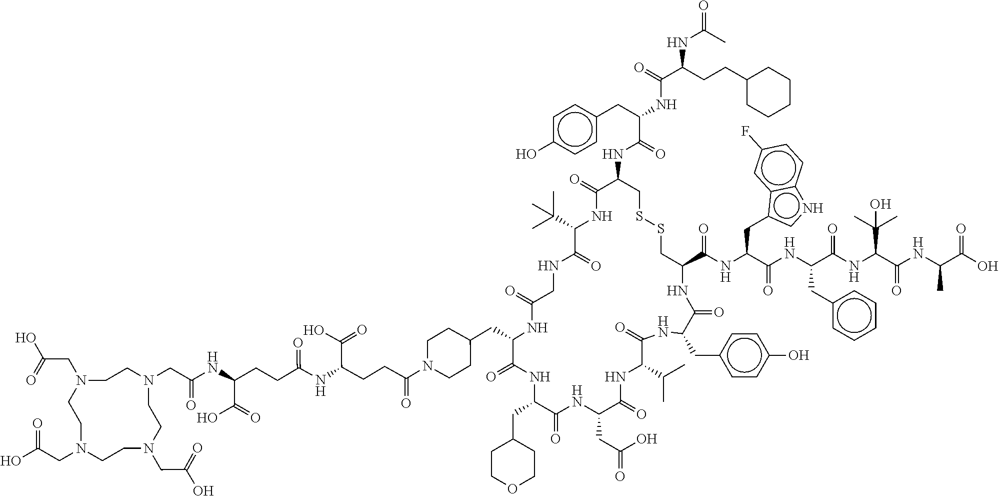 |
| 488 | 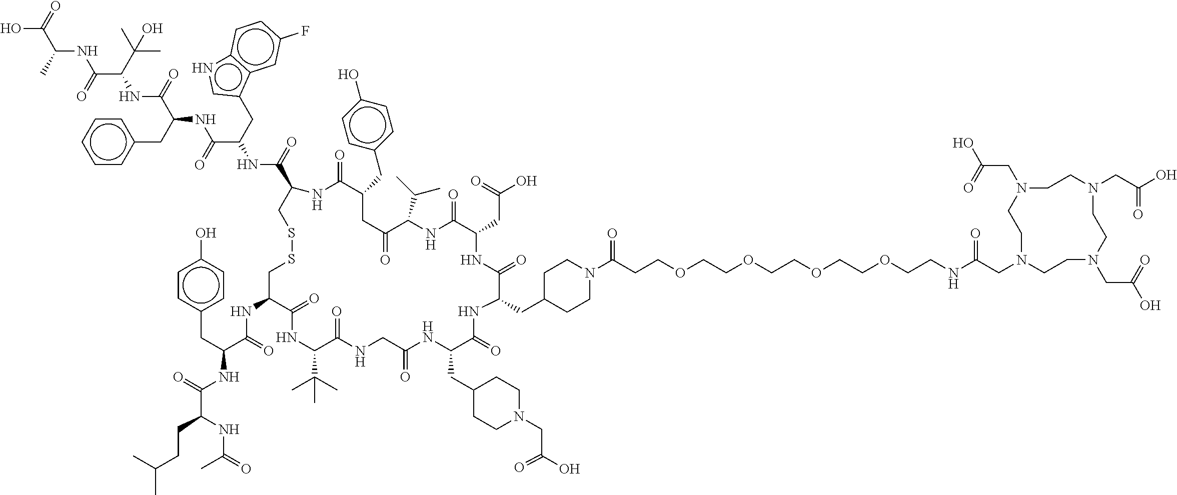 |
| 489 | 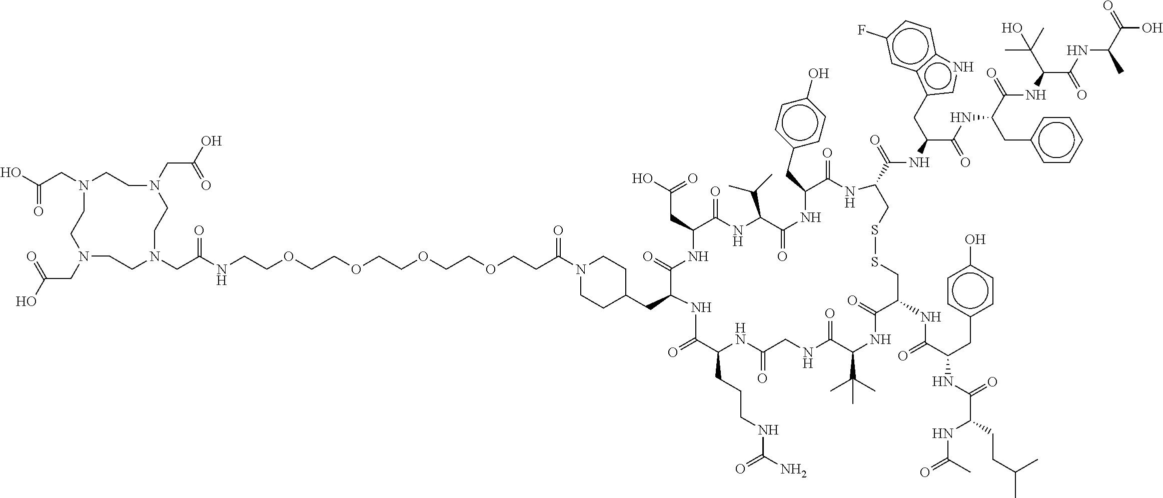 |
| 490 | 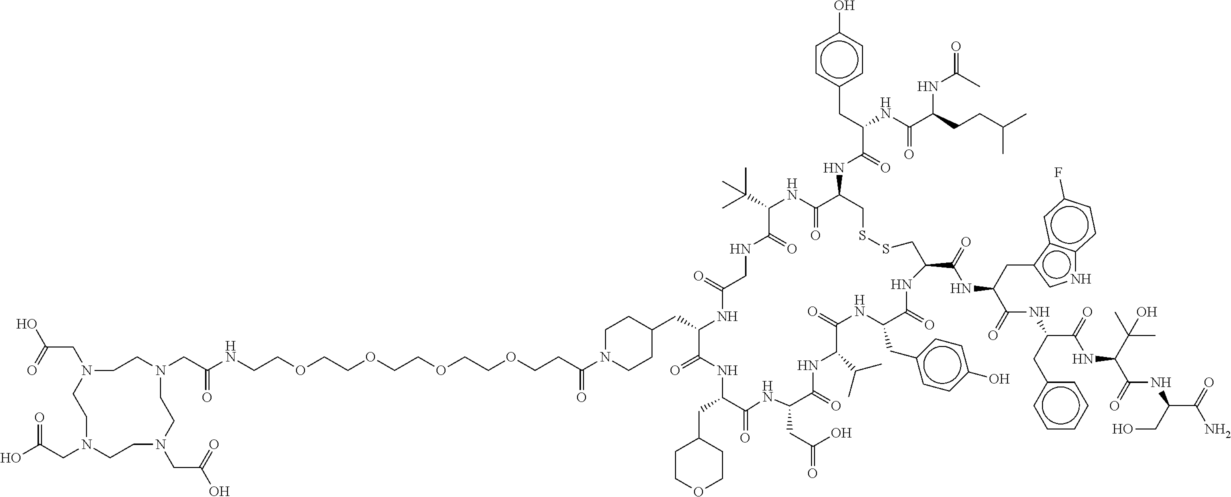 |
| 491 | 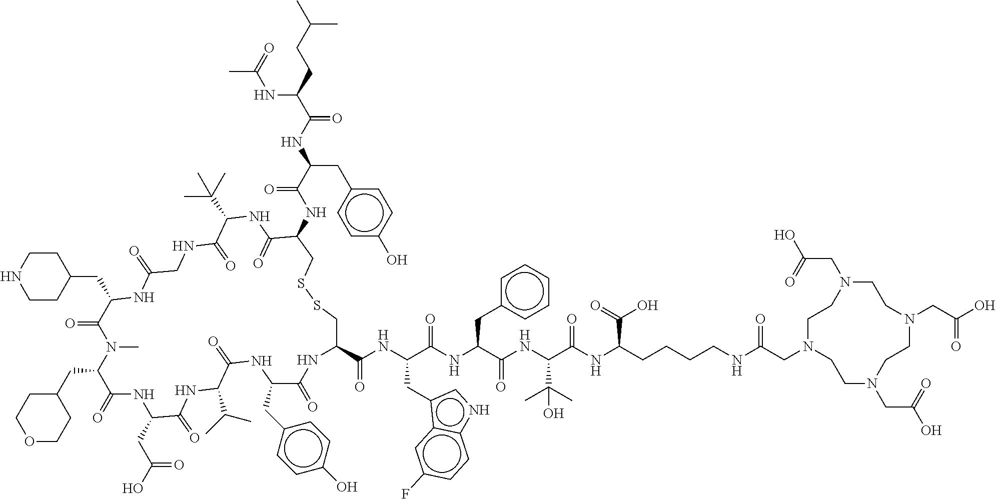 |
| 492 | 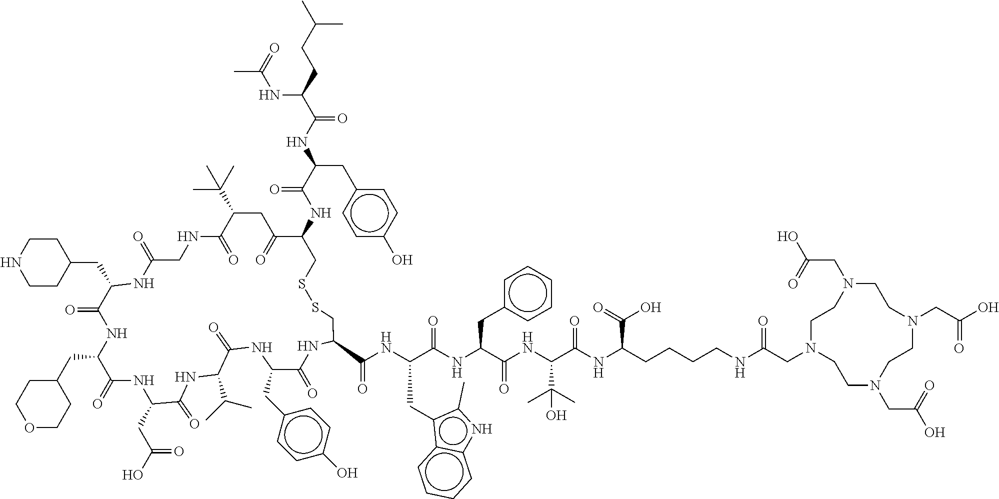 |
| 493 | 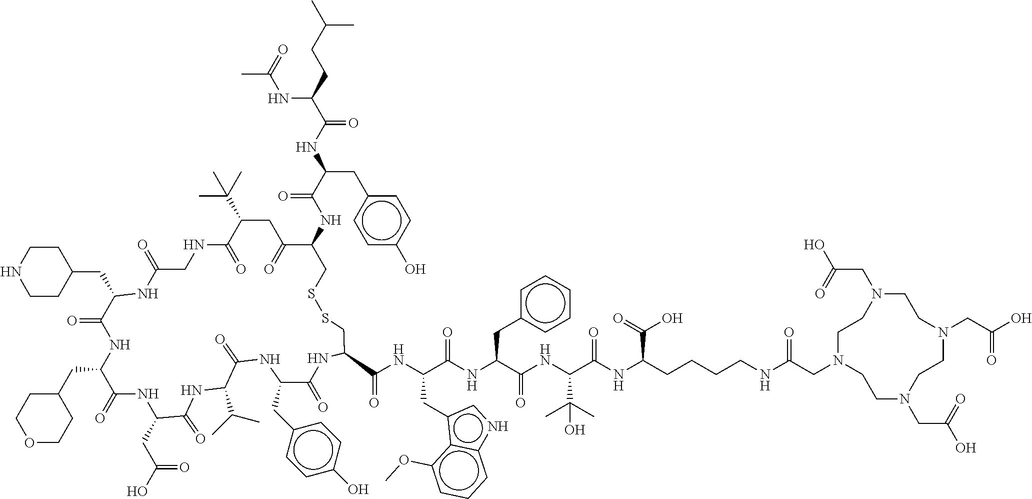 |
| 494 | 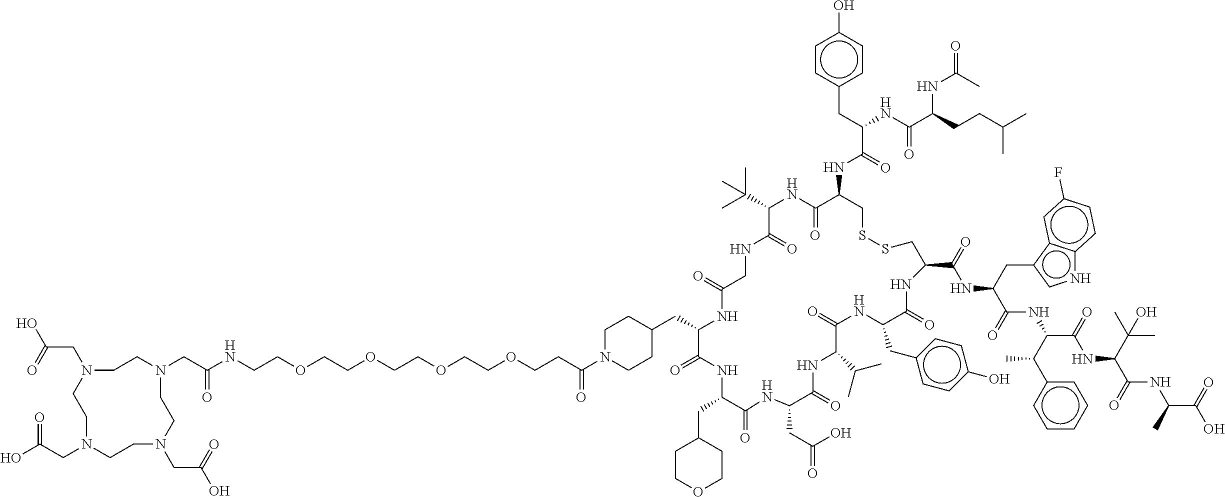 |
| 495 | 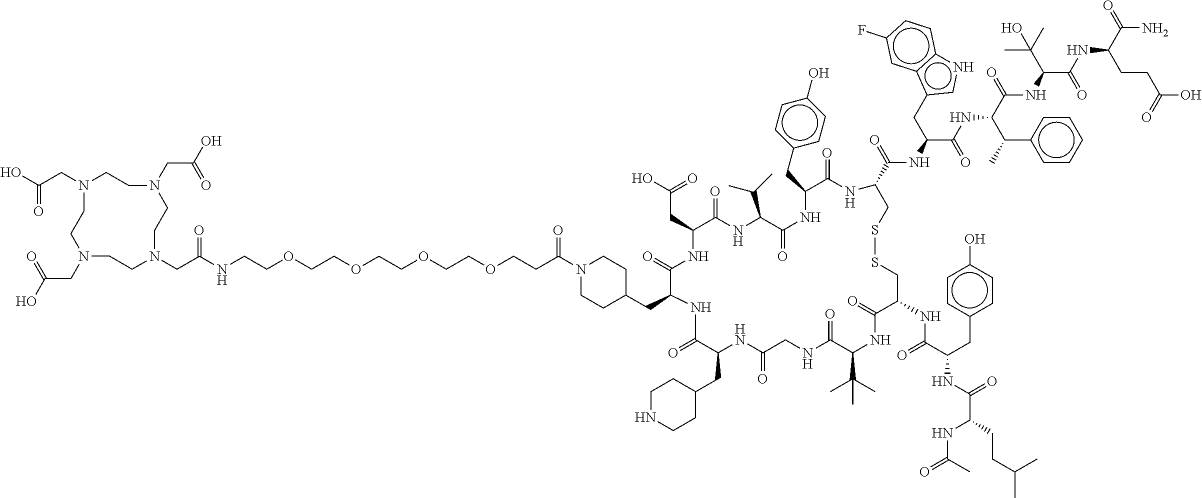 |
| 496 | 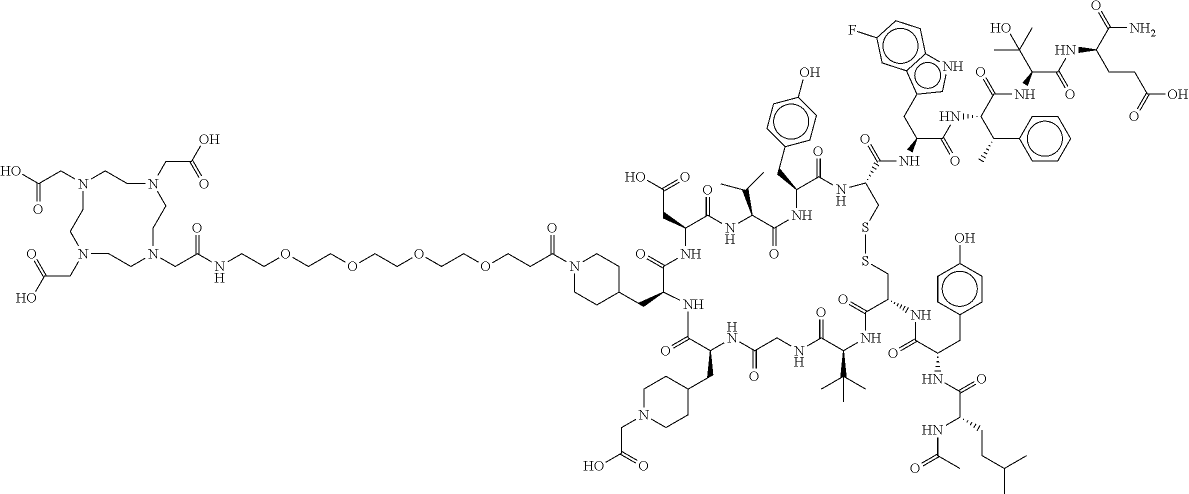 |
| 497 | 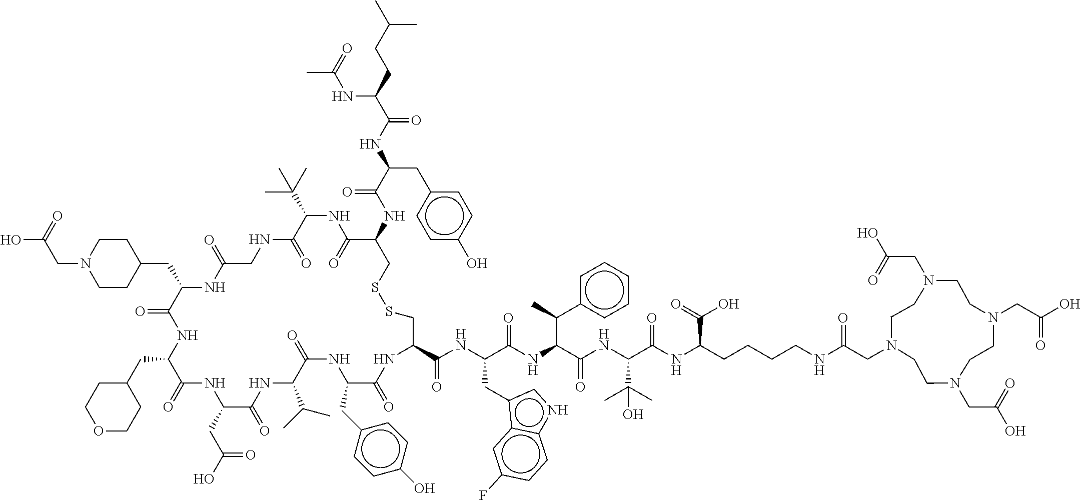 |
| 498 | 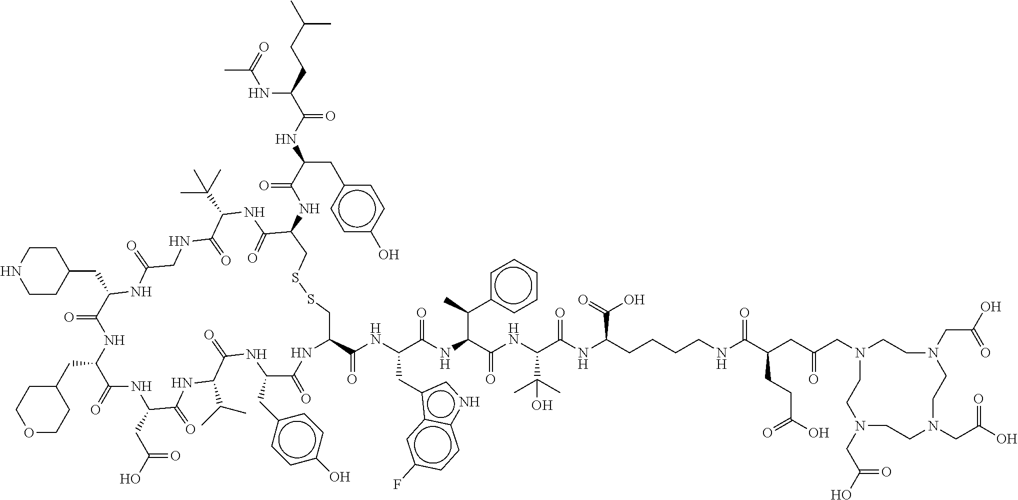 |
| 499 | 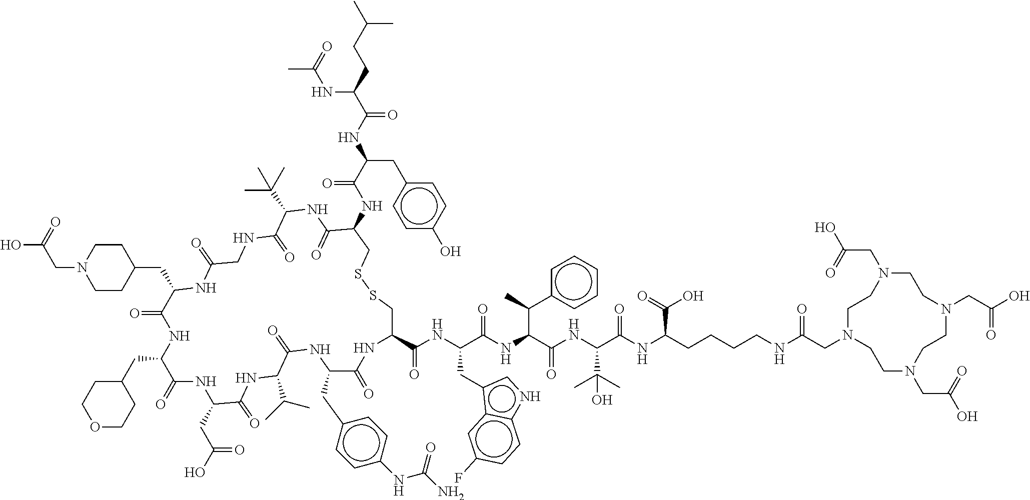 |
| 500 | 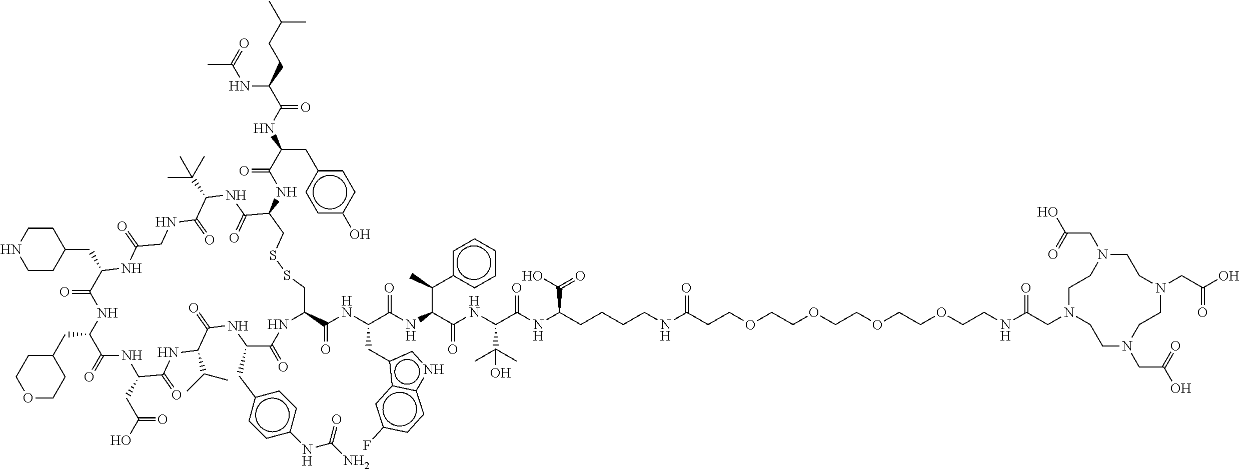 |
| 501 | 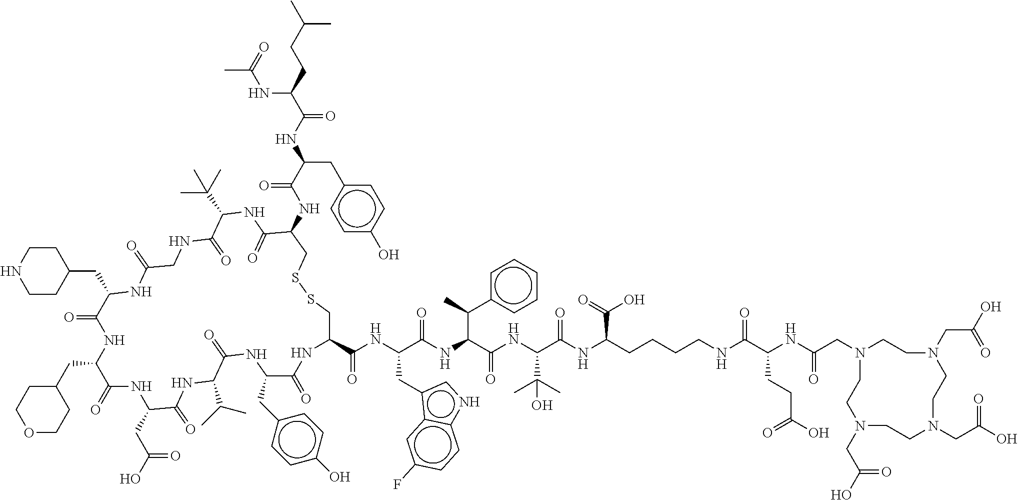 |
| 502 | 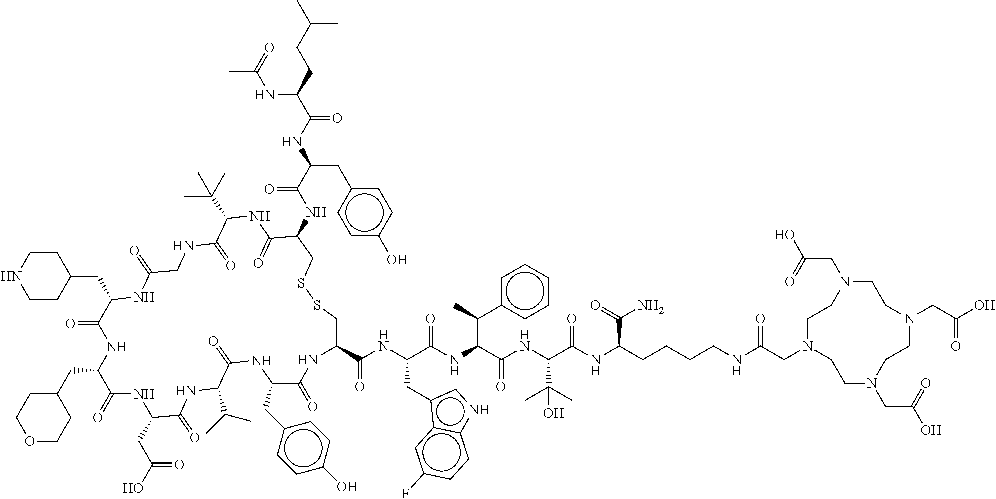 |
| 503 | 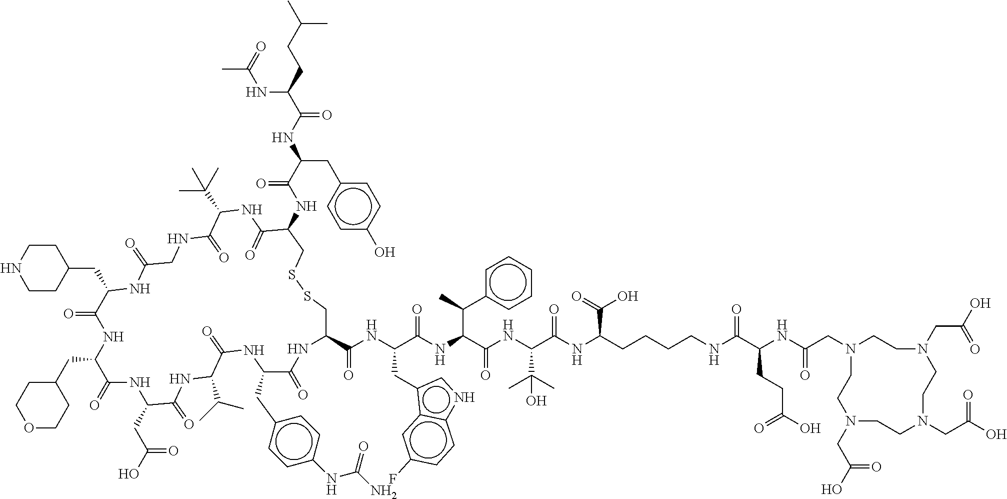 |
| 504 | 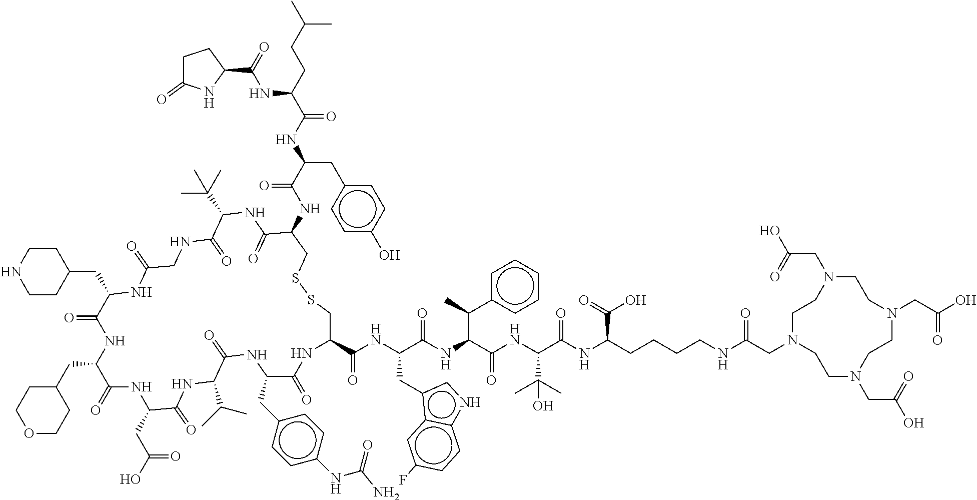 |
| 505 | 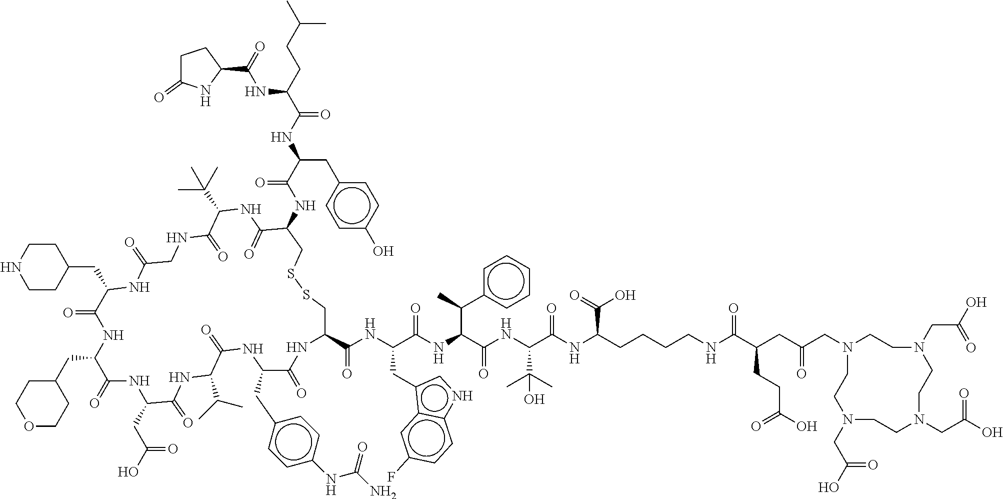 |
| 506 | 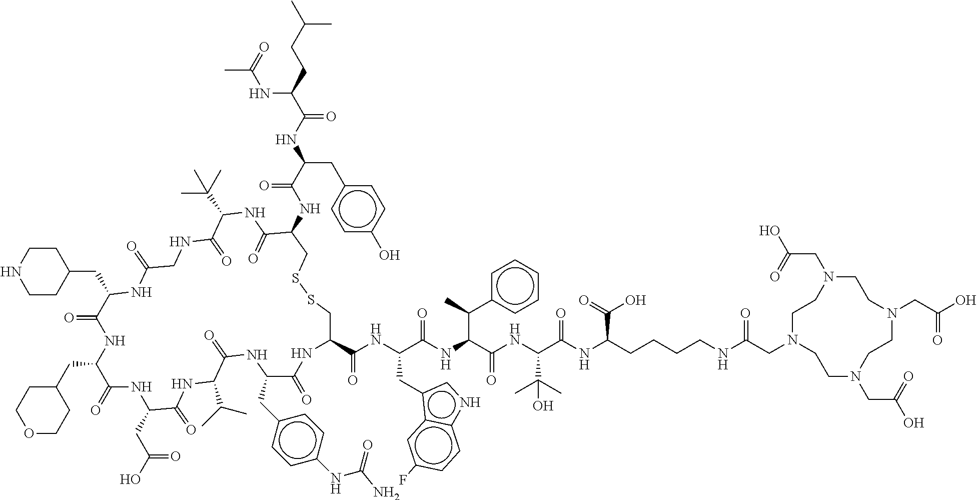 |
| 507 | 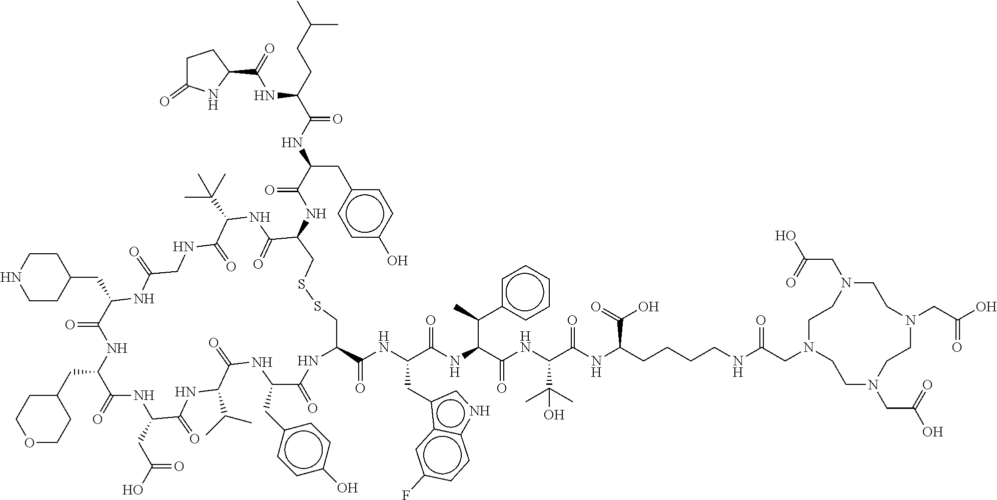 |
| 508 | 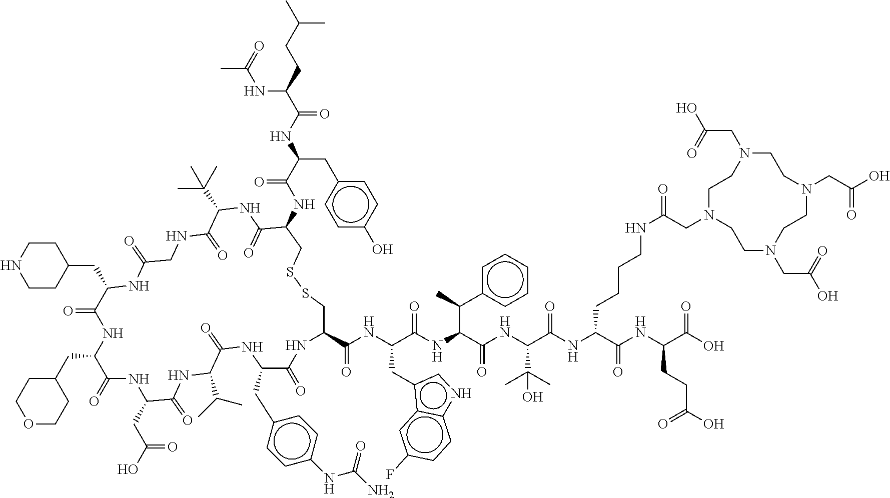 |
| 509 | 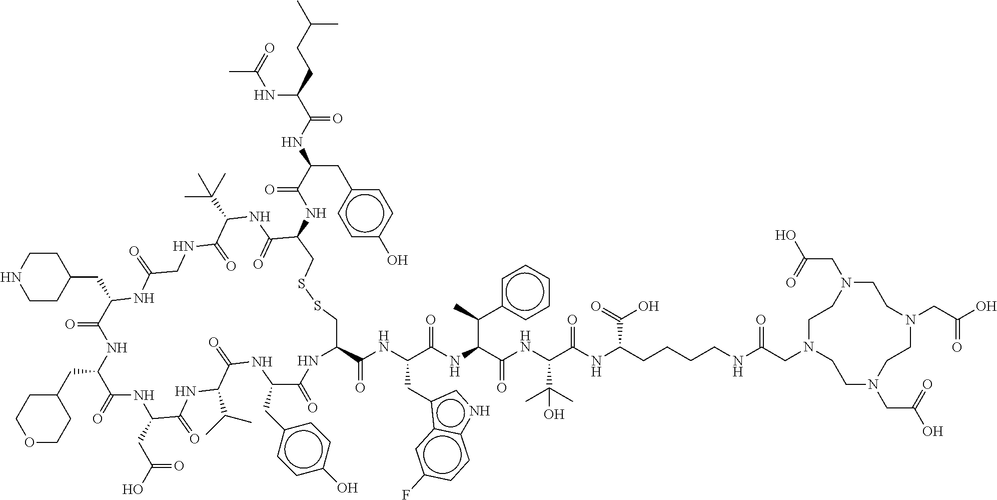 |
| 510 | 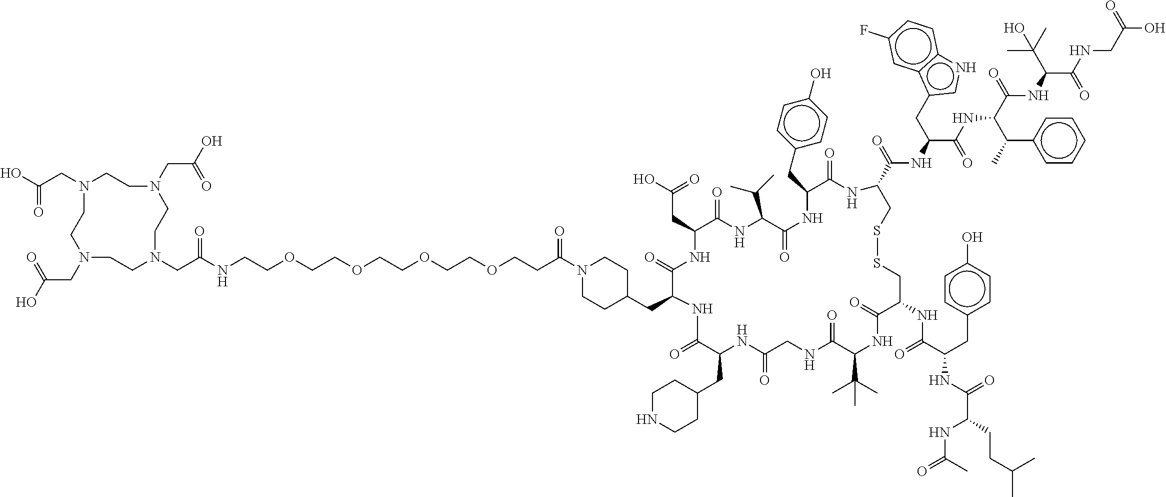 |
| 511 | 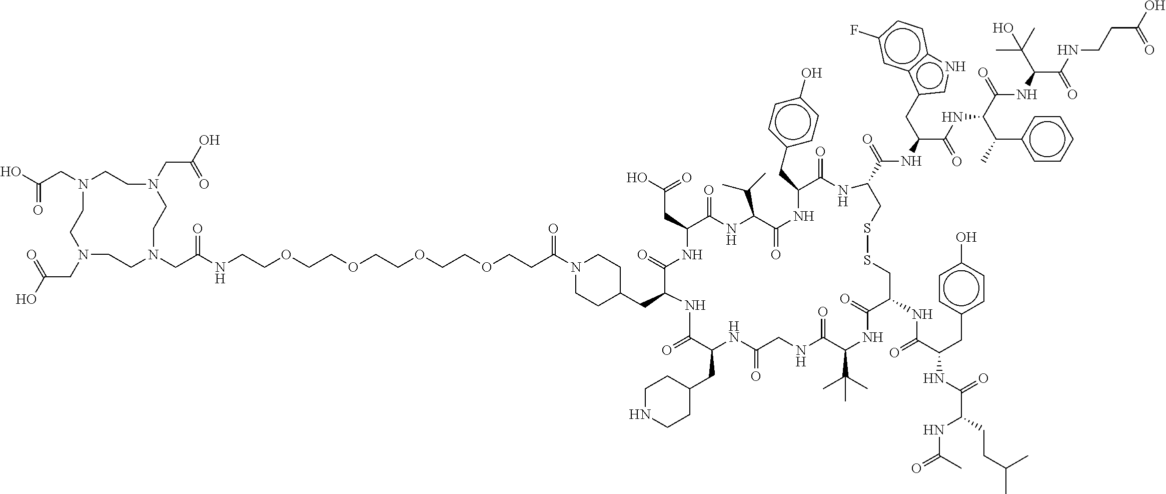 |
| 512 | 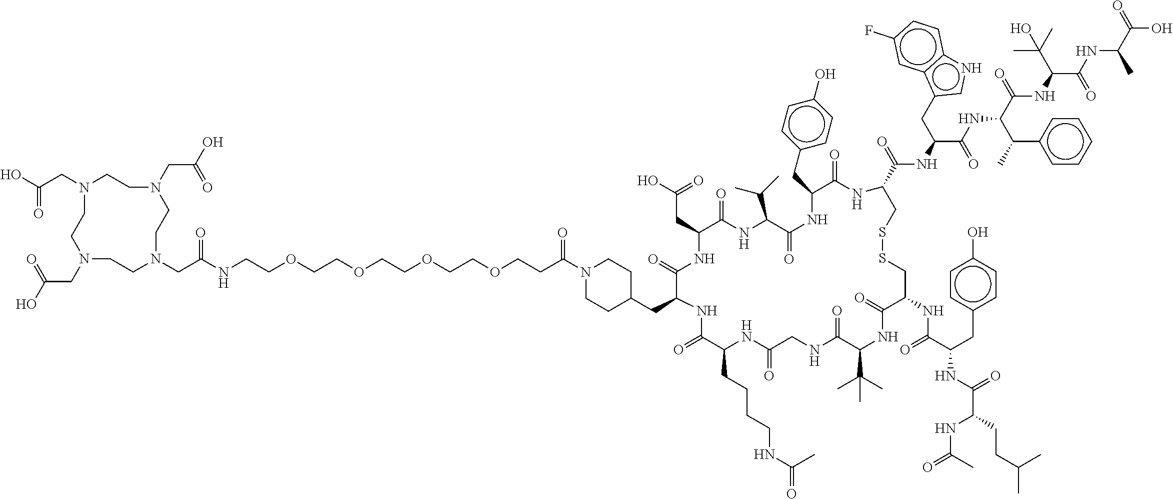 |
| 513 | 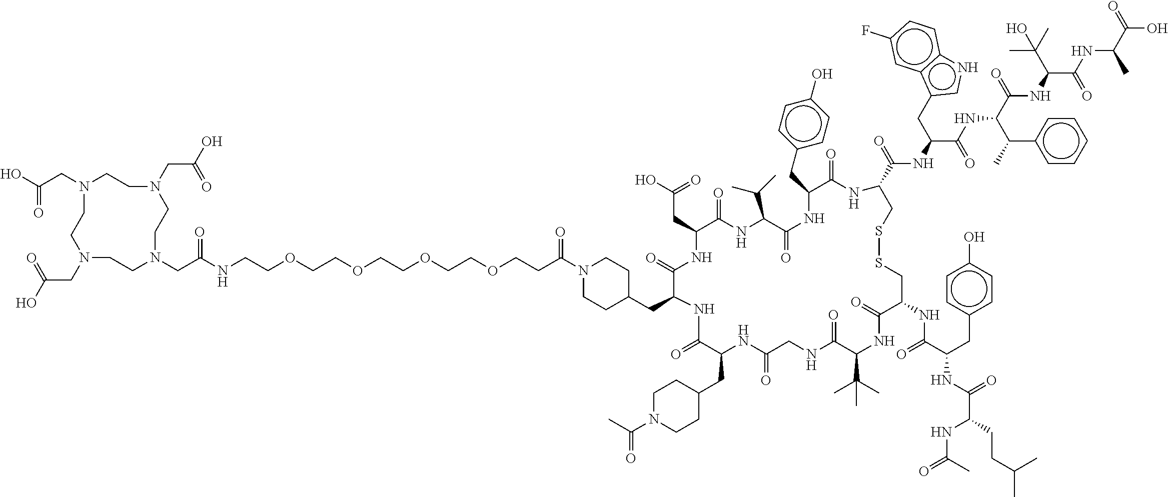 |
| 514 | 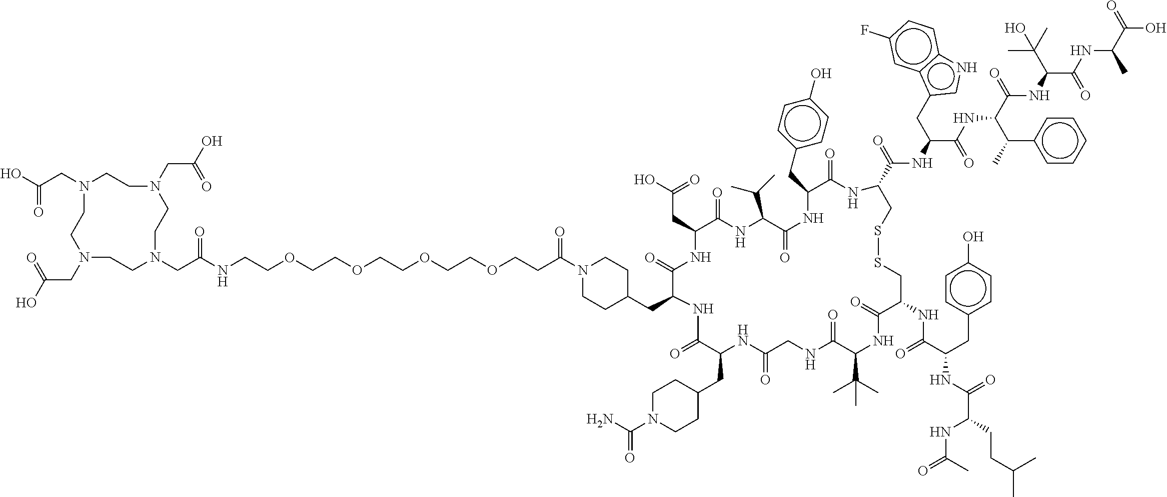 |
| 515 | 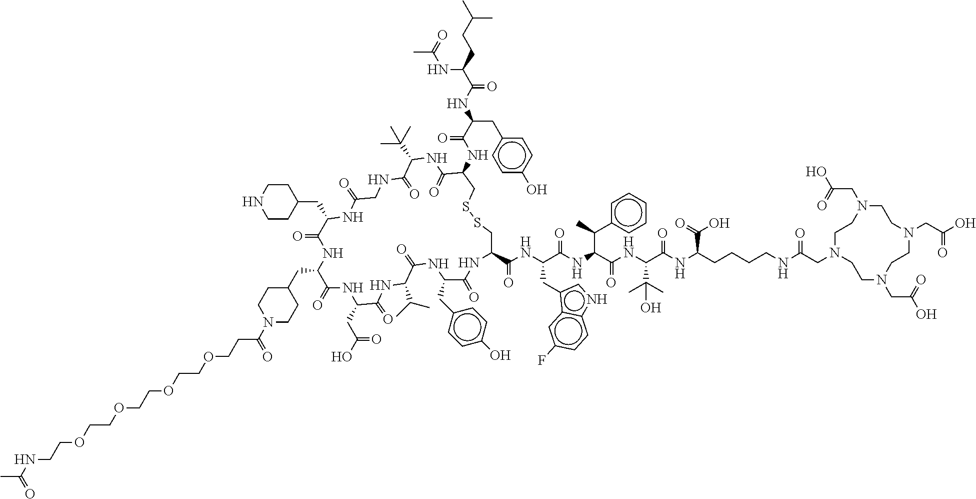 |
| 516 | 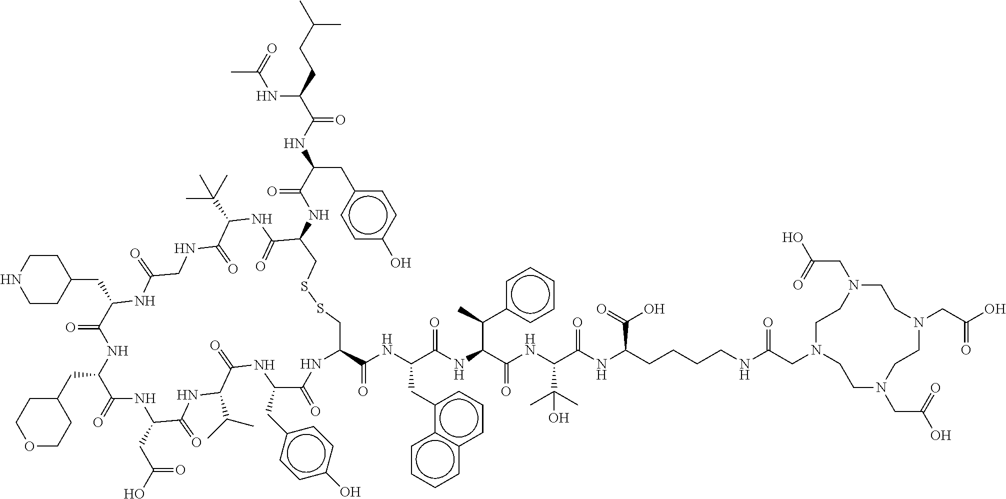 |
| 517 | 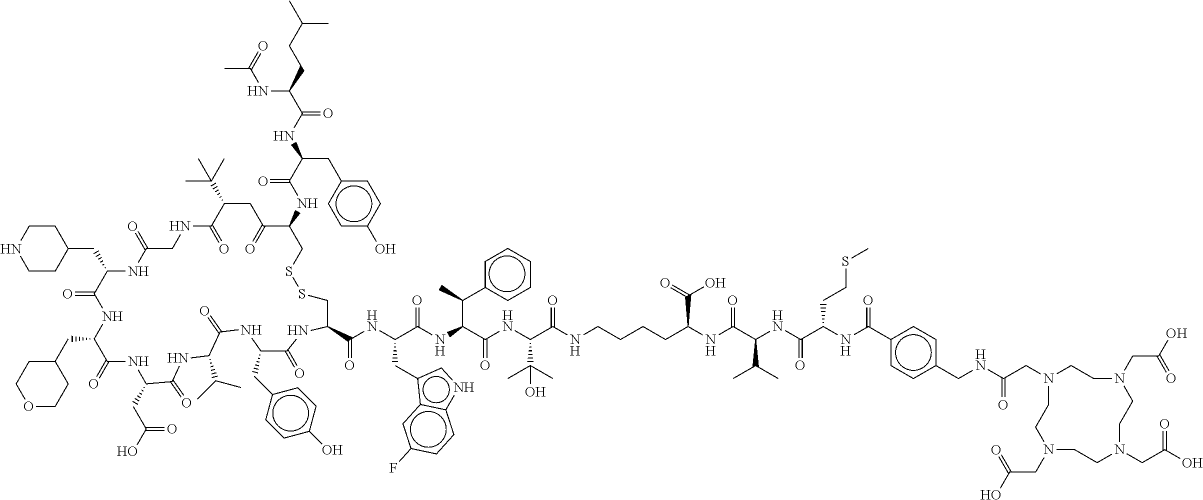 |
| 518 | 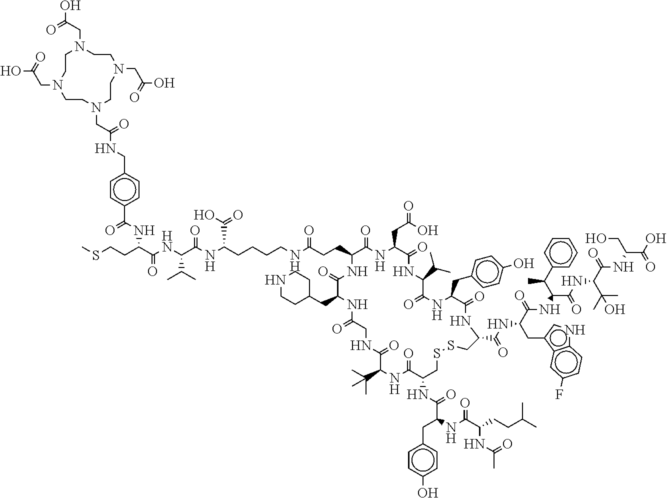 |
| 519 | 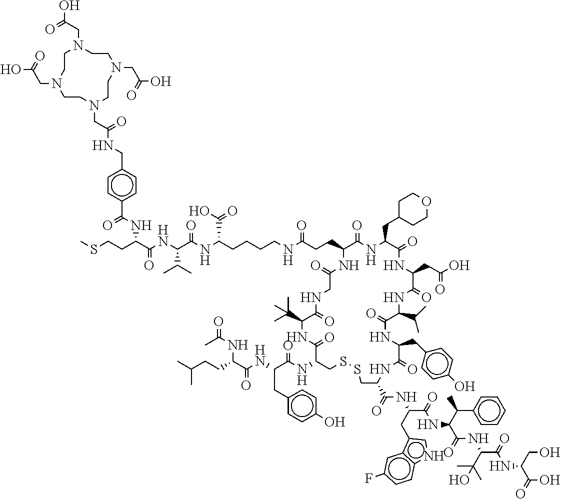 |
| 520 | 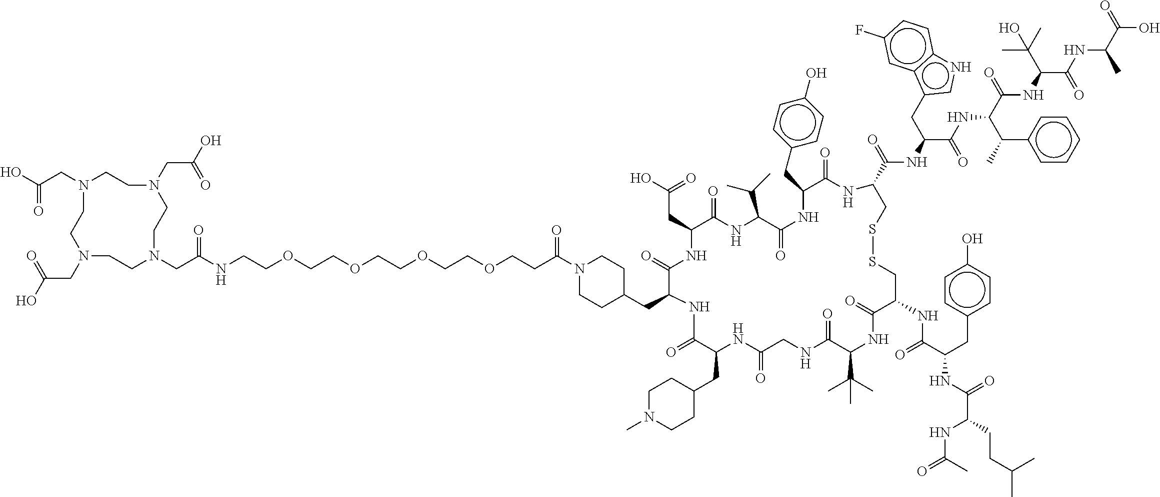 |
| 521 | 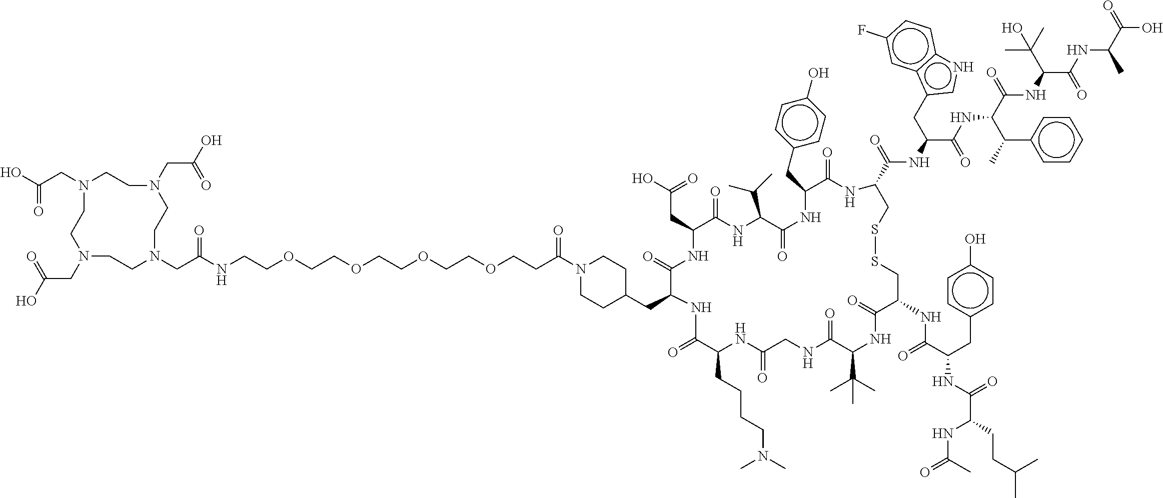 |
| 522 | 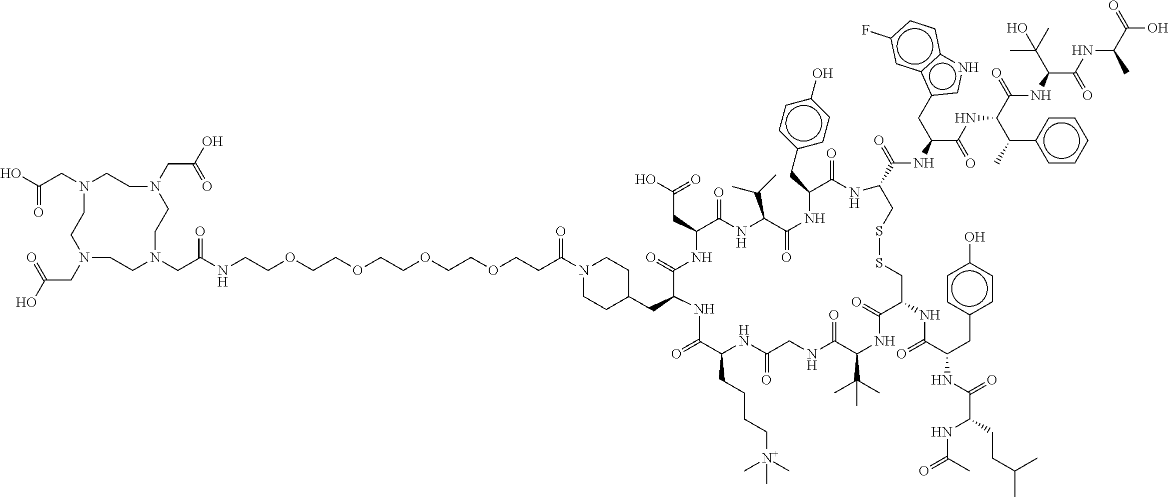 |
| 523 | 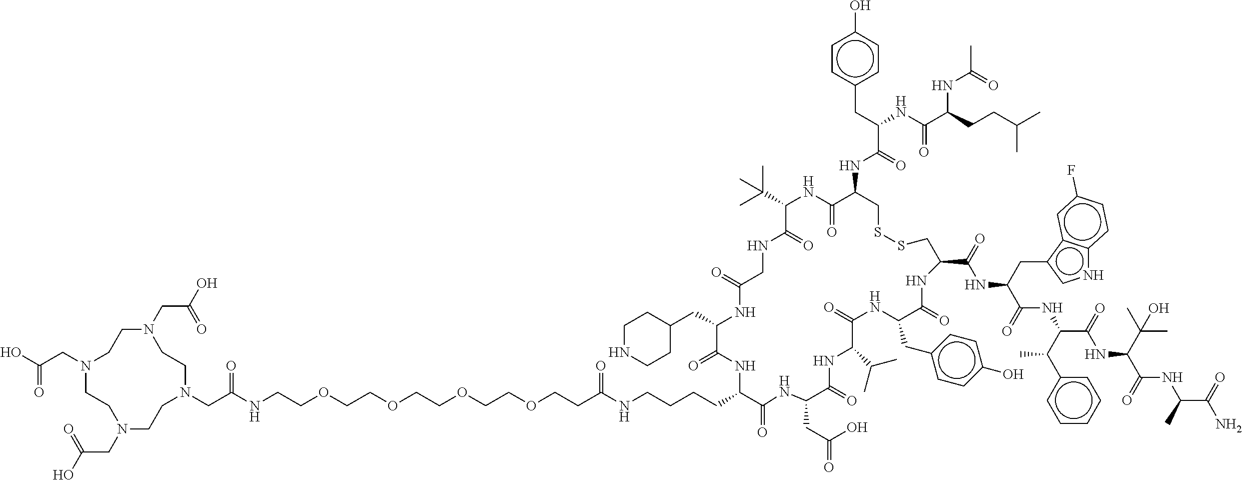 |
| 524 | 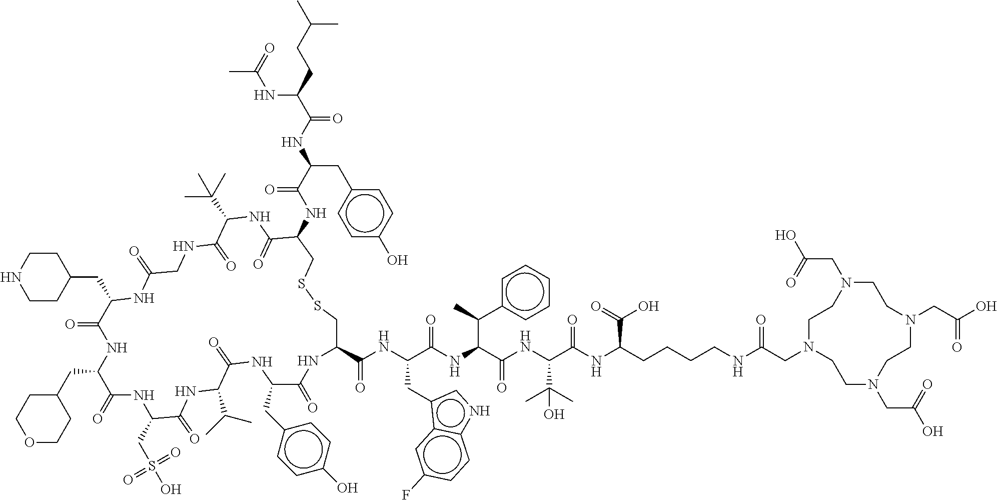 |
| 525 | 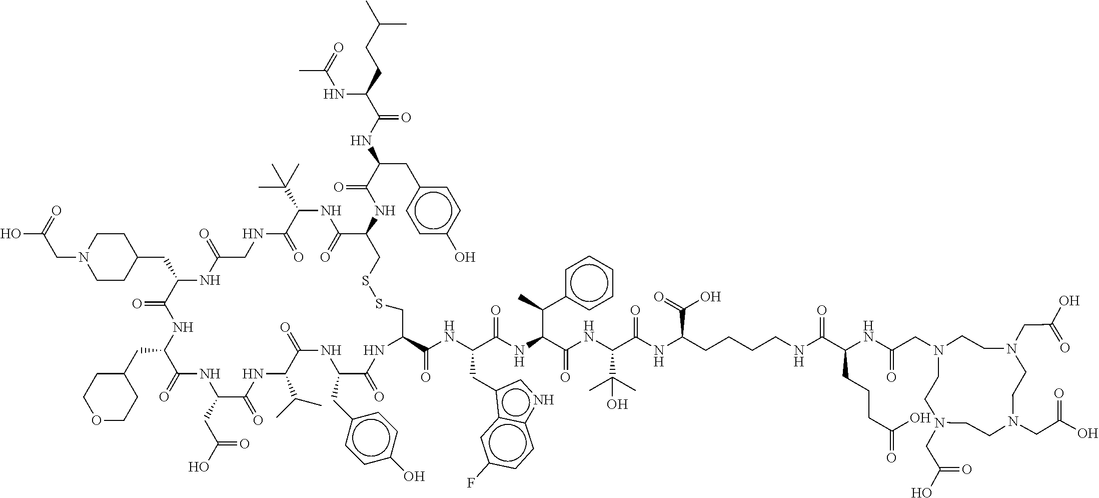 |
| 526 | 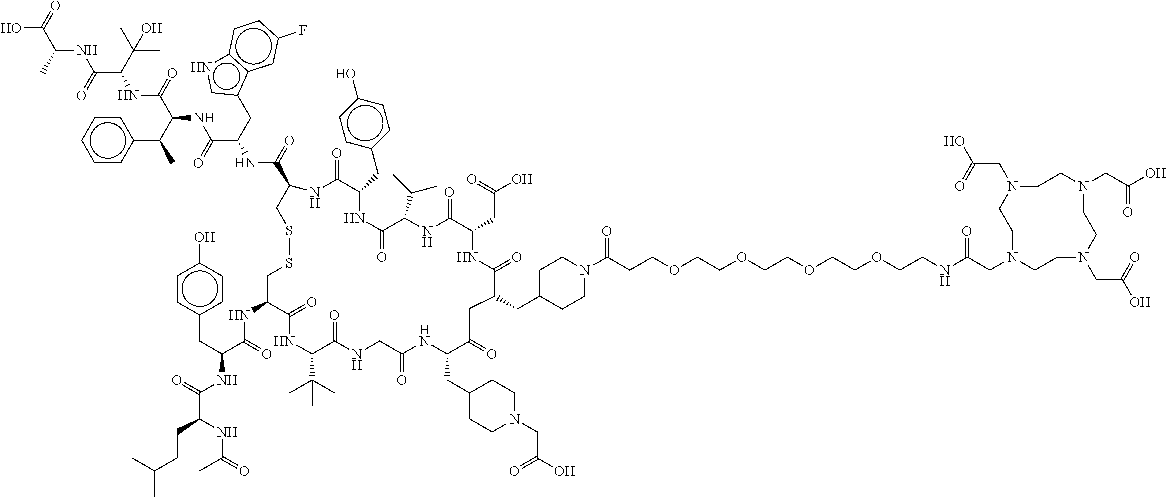 |
| 527 | 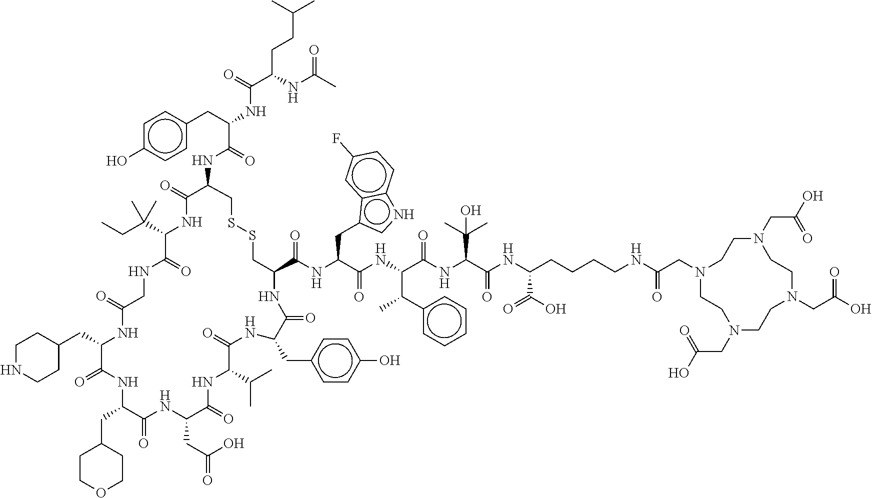 |
| 528 | 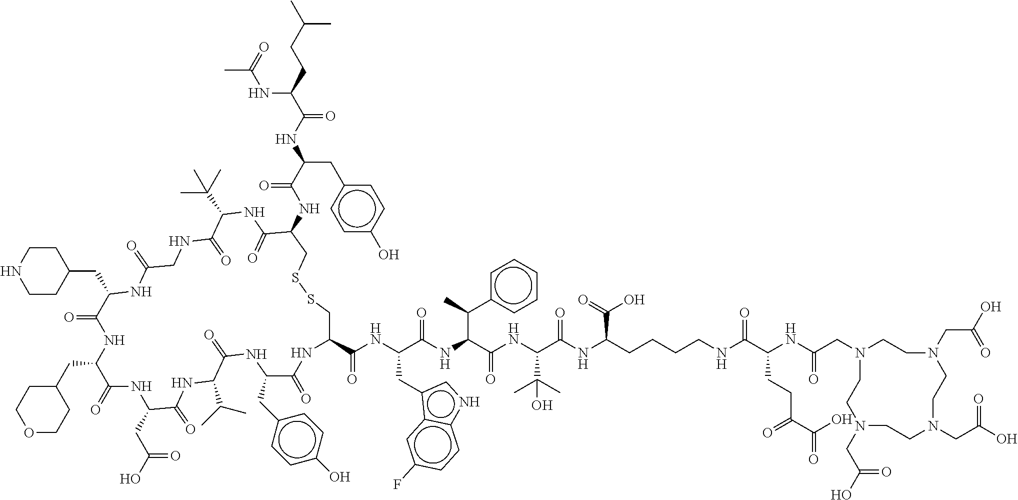 |
| 529 | 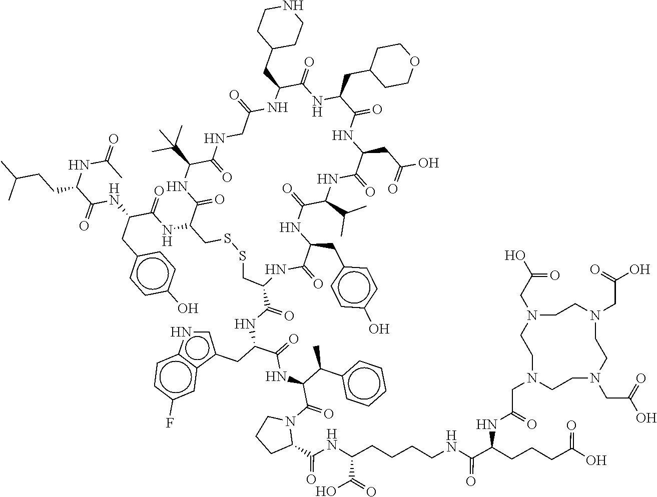 |
| 530 | 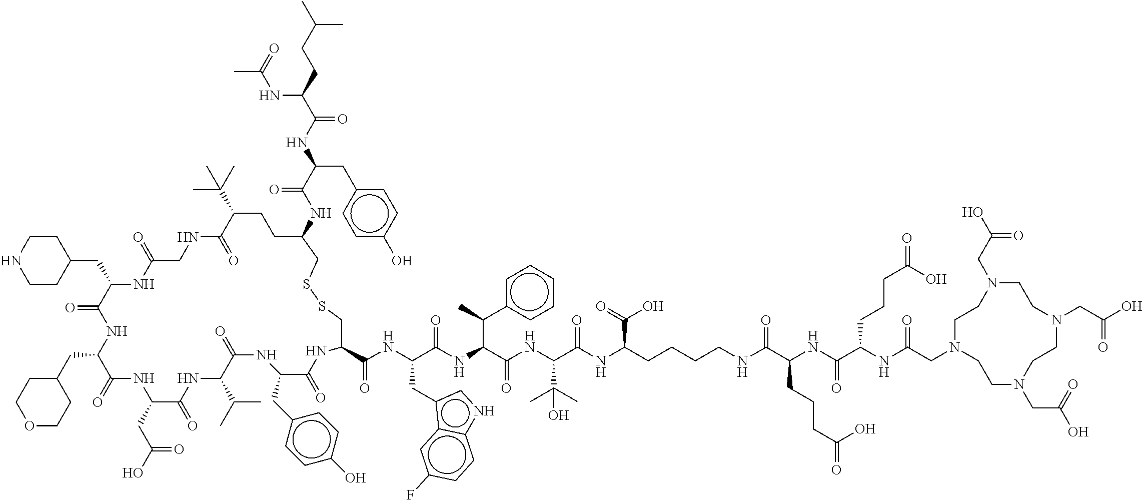 |
| 531 | 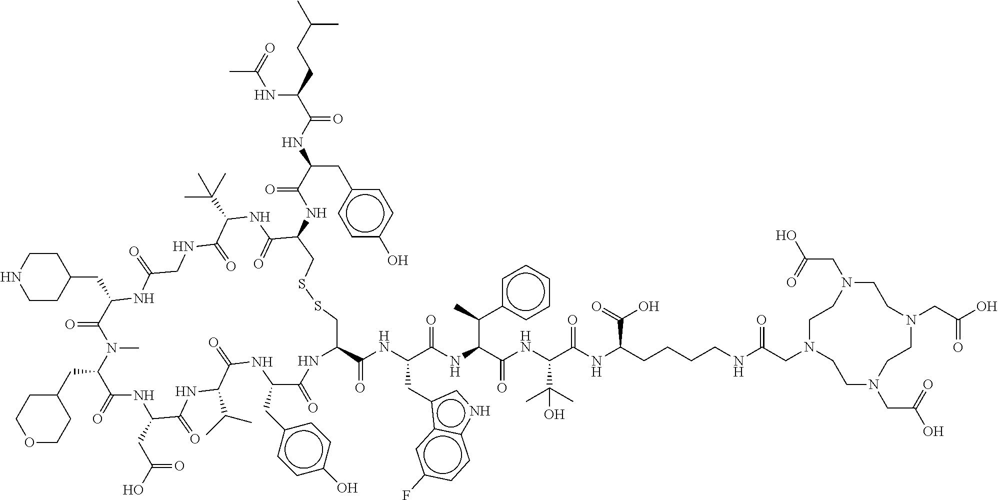 |
| 532 | 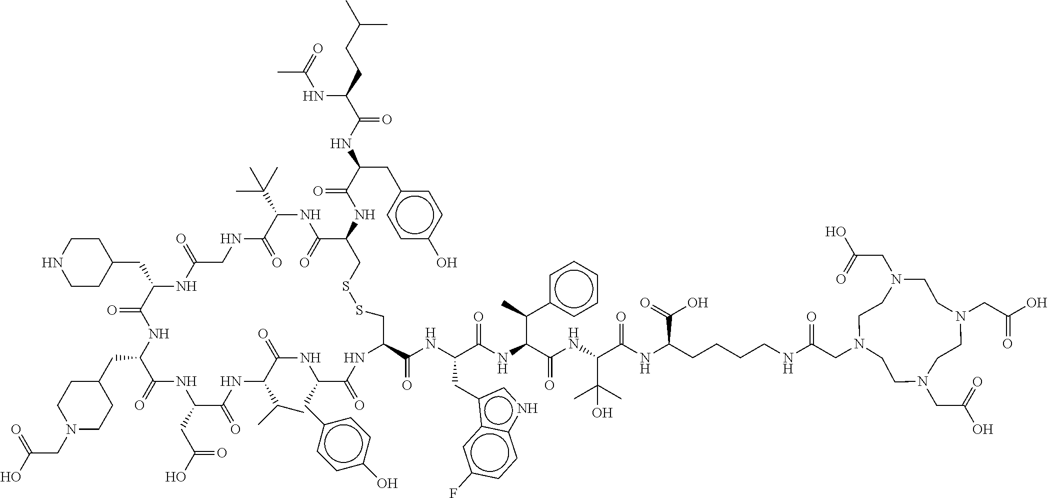 |
| 533 | 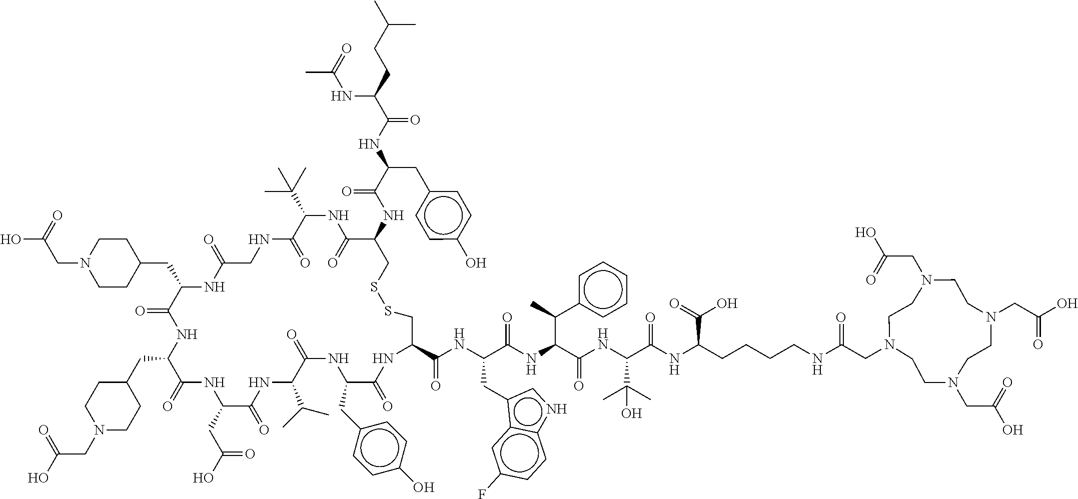 |
| 534 | 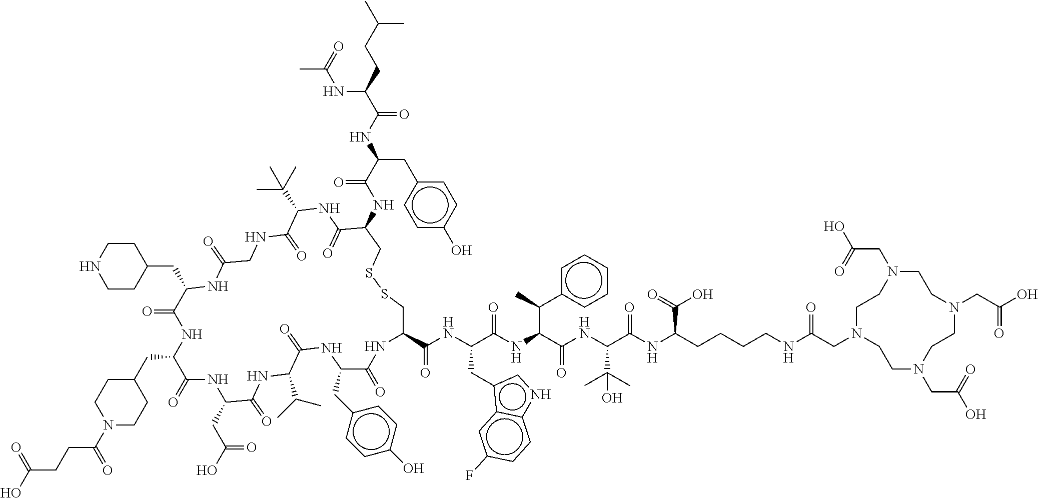 |
| 535 | 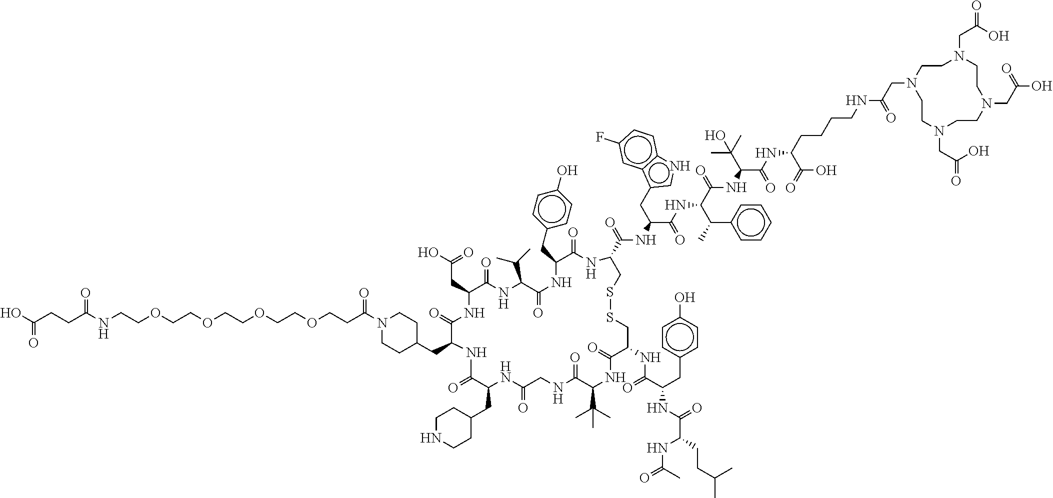 |
| 536 | 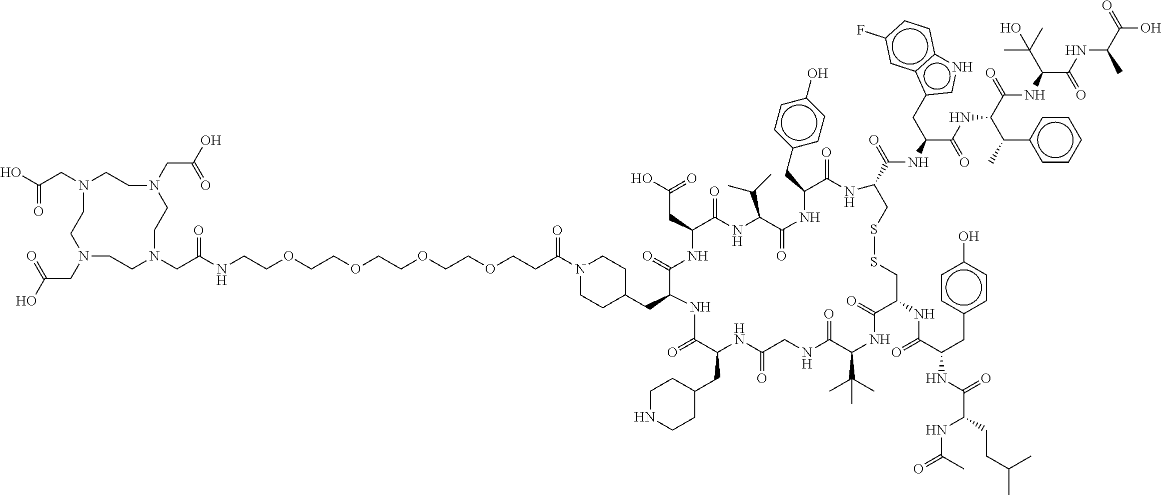 |
| 537 | 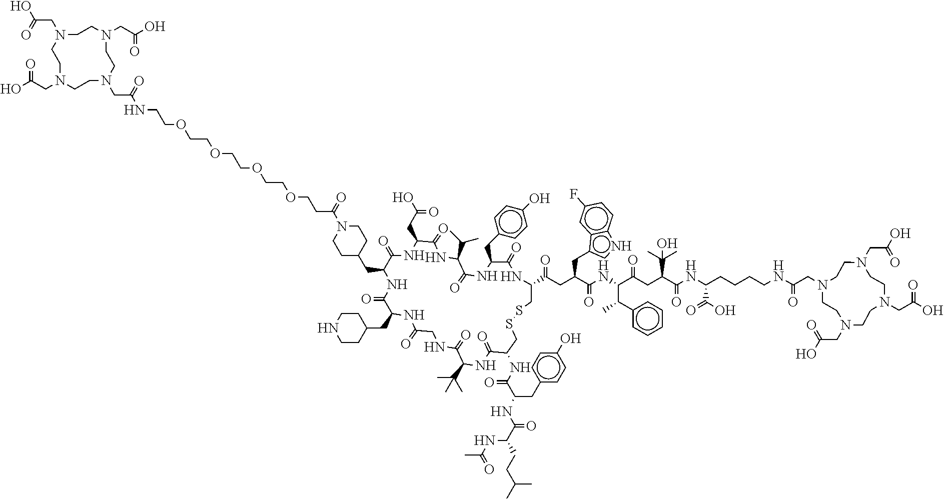 |
| 538 | 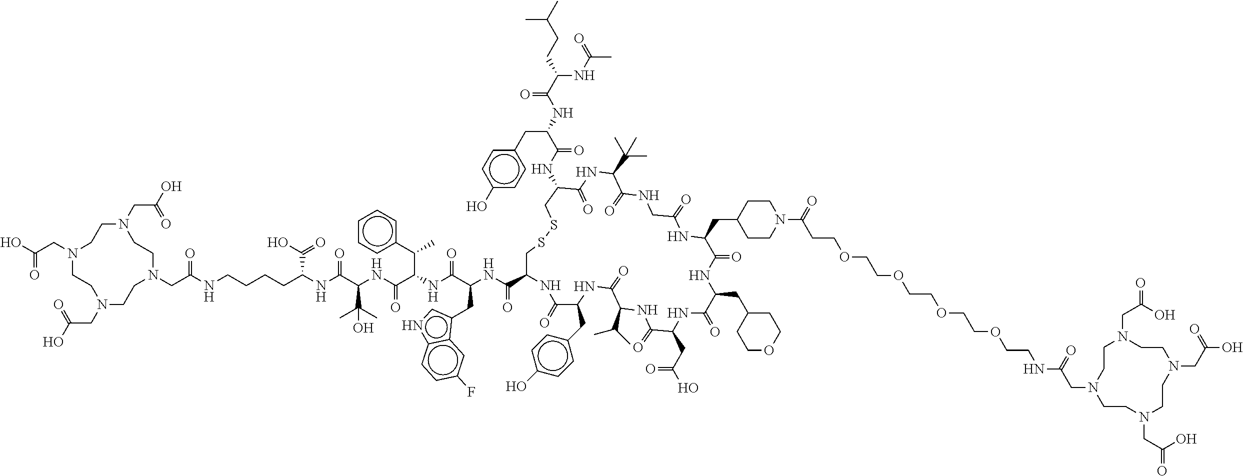 |
| 539 | 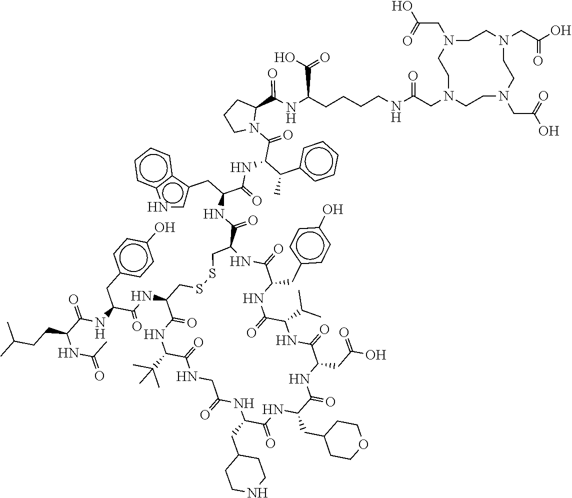 |
| 540 | 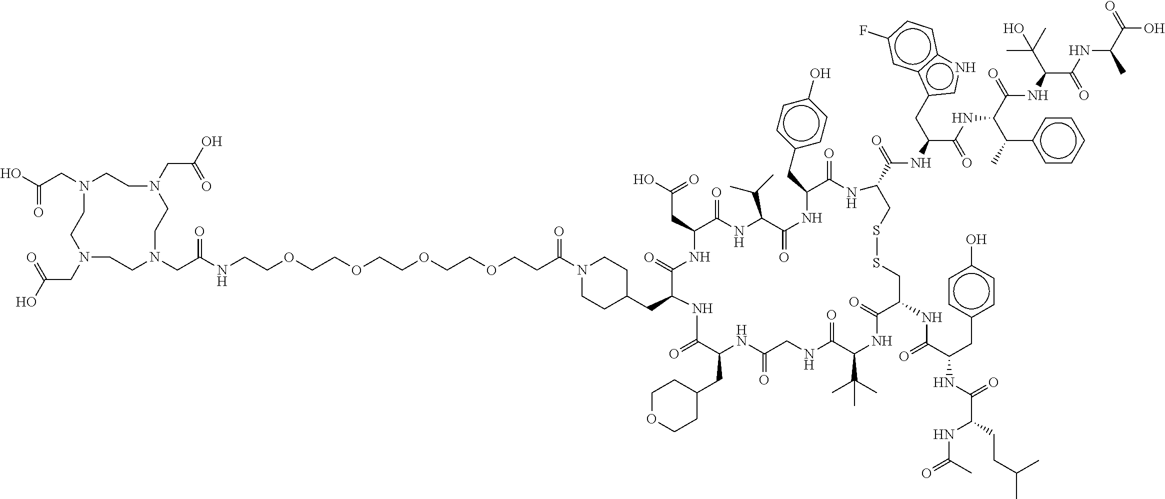 |
| 541 | 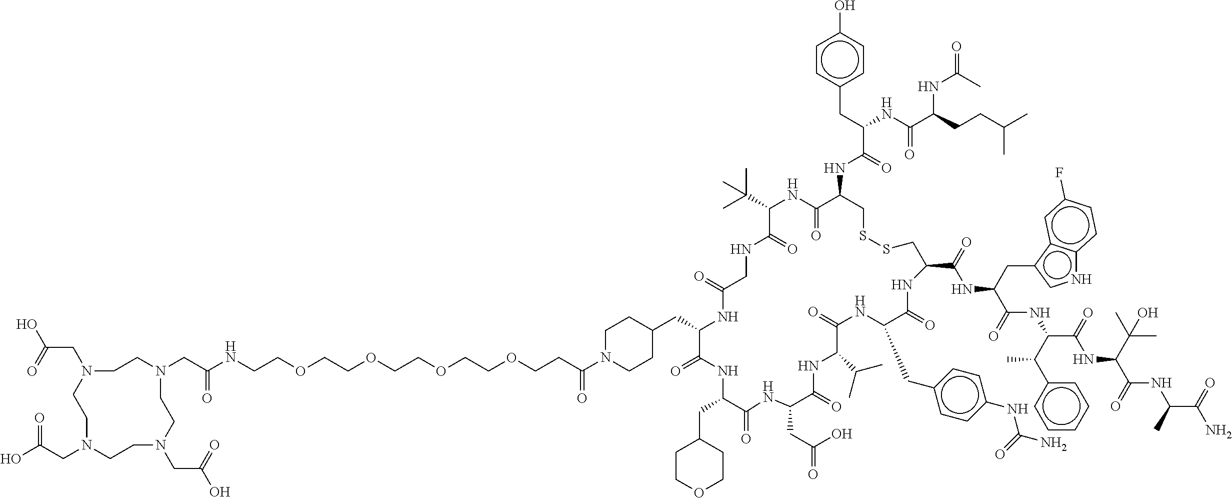 |
| 542 | 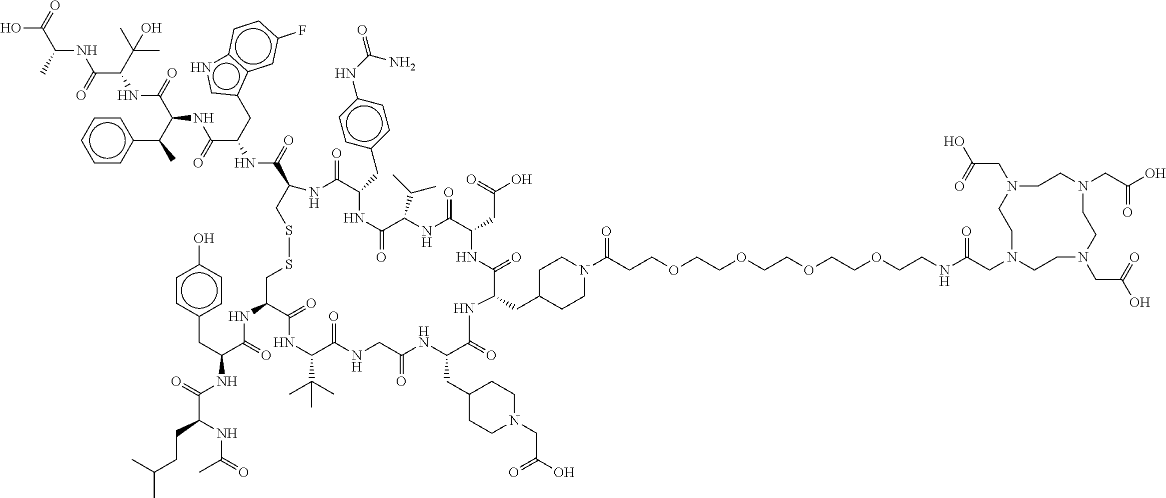 |
| 543 | 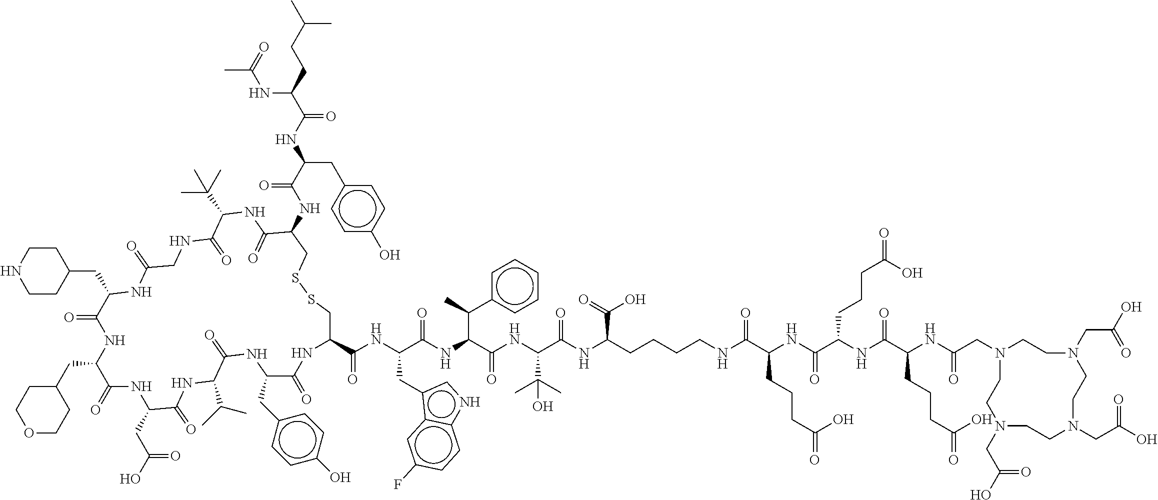 |
| 544 | 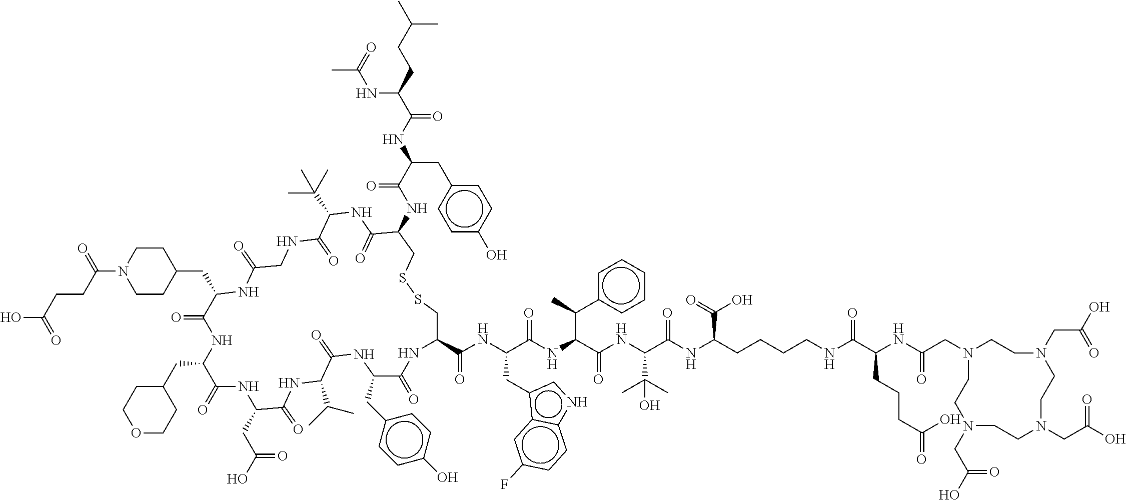 |
| 545 | 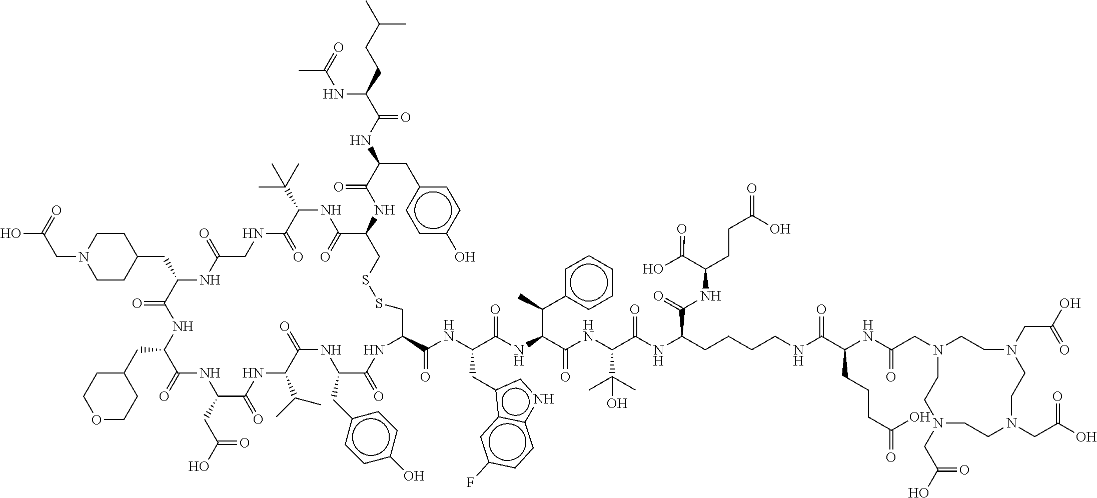 |
| 546 | 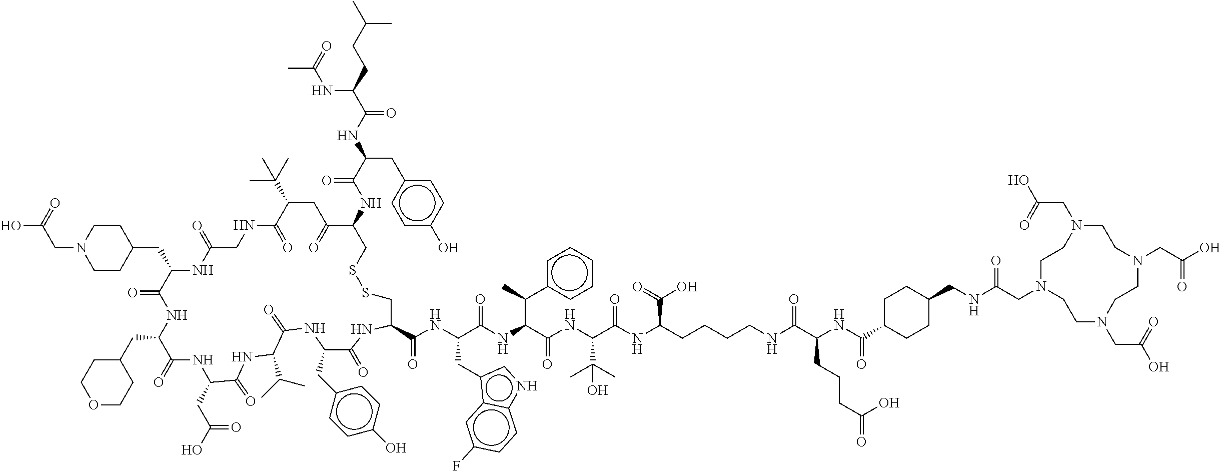 |
| 547 | 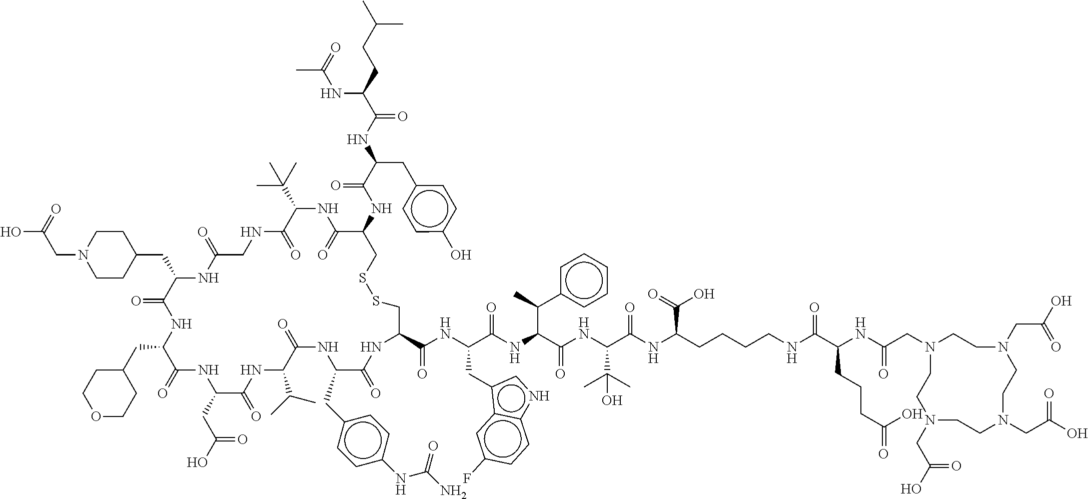 |
| 548 | 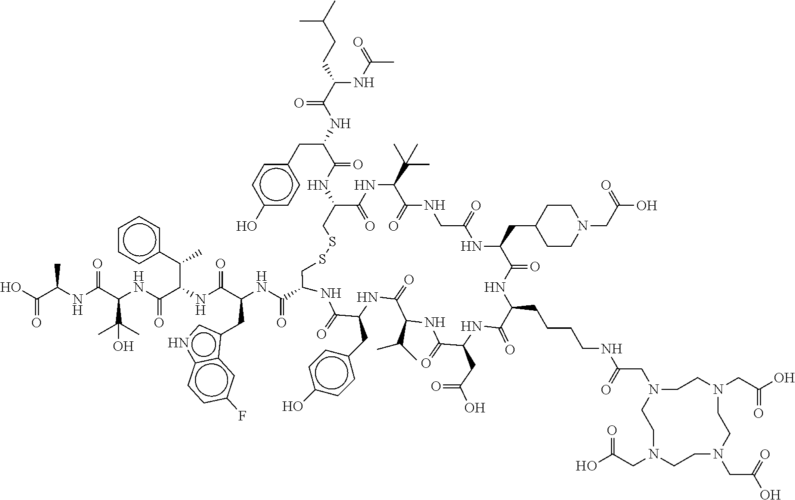 |
| 549 | 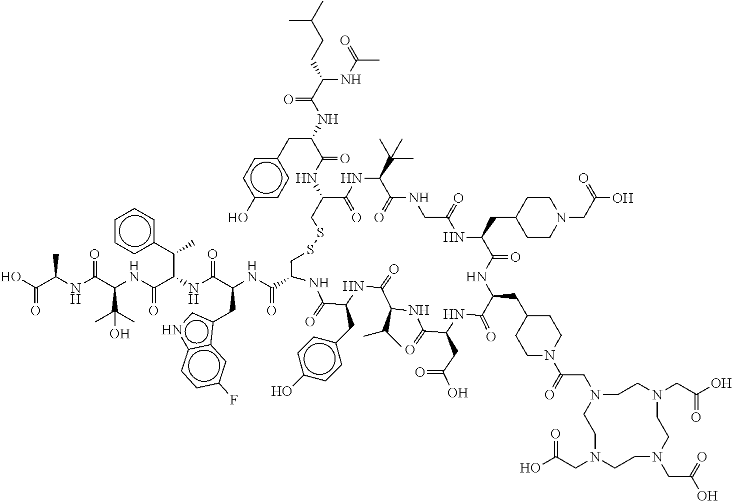 |
| 550 | 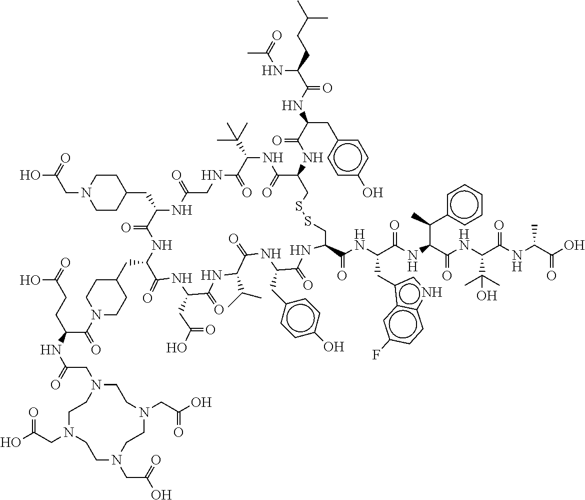 |
| 551 | 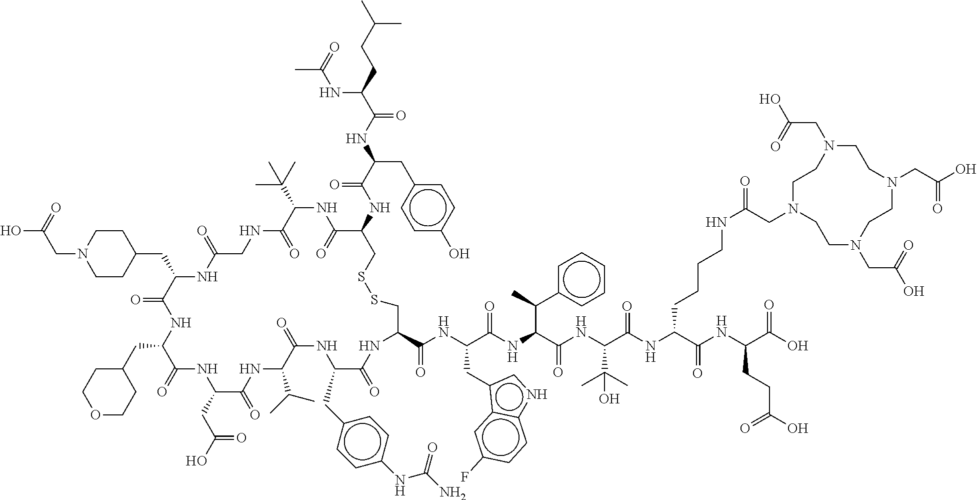 |
| 552 | 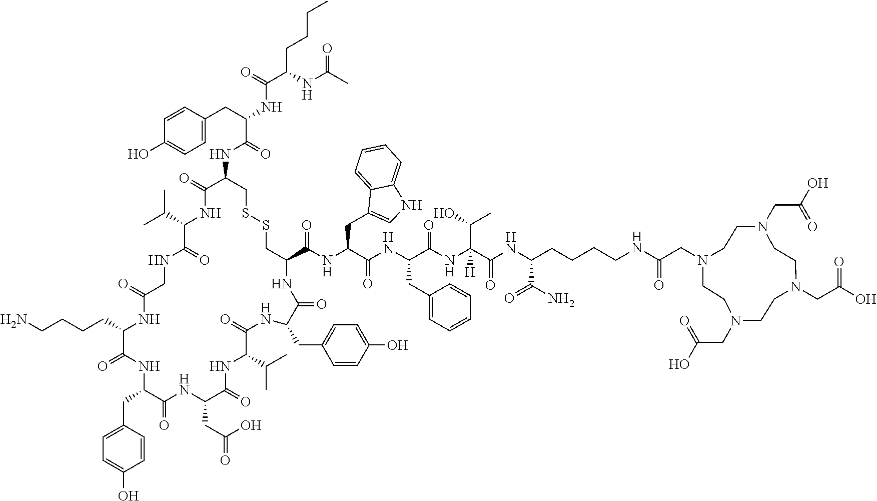 [SMILES: CCCC[C@H](NC(C)=O)C(=O)N[C@@H](Cc1ccc(O)cc1)C(=O)N[C@H]1CSSC[C@@H](C(=O)N[C@@H](Cc2c[nH]c3ccccc23)C(=O)N[C@@H](Cc2ccccc2)C(=O)N[C@H](C(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCN)NC(=O)CNC(=O)[C@H](C(C)C)NC1=O] |

or a pharmaceutically acceptable salt thereof.

In yet another embodiment, the cyclic peptide of Formula I or Formula VI is selected from a peptide in Table 2.

TABLE 2

| | | SEQ |
|---|---|---|
| No | Sequence | ID NO: |
| 1 | Ac-M-Y-C-V-G-K-Y-D-V-Y-C-W-F-CONH2 | 1 |
| 2 | Ac-M-Y-C-V-G-K-Y-D-V-Y-C-W-F-dLys(DOTA)-CONH2 | 2 |
| 3 | DOTA-PEG8-M-Y-C-V-G-K-Y-D-V-Y-C-W-F-CONH2 | 3 |
| 4 | Ac-Nle-Y-C-V-G-K-Y-D-V-Y-C-W-F-dLys(DOTA)-CONH2 | 4 |
| 5 | DOTA-PEG8-Nle-Y-C-V-G-K-Y-D-V-Y-C-W-F-CONH2 | 5 |
| 6 | Ac-M-Y-C-V-G-K-Y-D-V-Y-C-W-F-COOH | 6 |
| 7 | Ac-Nle-Y-C-V-G-K-Y-D-V-Y-C-W-F-CONH2 | 7 |
| 8 | Ac-M-dA-C-V-G-K-Y-D-V-Y-C-W-F-CONH2 | 8 |
| 9 | Ac-M-Y-C-V-dAla-K-Y-D-V-Y-C-W-F-CONH2 | 9 |
| 10 | Ac-M-Y-C-V-G-K-Ala-D-V-Y-C-W-F-CONH2 | 10 |
| 11 | Ac-M-Y-C-V-G-K-dAla-D-V-Y-C-W-F-CONH2 | 11 |
| 12 | Ac-M-Y-C-V-G-K-Y-Ala-V-Y-C-W-F-CONH2 | 12 |
| 13 | Ac-M-Y-C-V-G-K-Y-dAla-V-Y-C-W-F-CONH2 | 13 |
| 14 | Ac-M-Y-C-V-G-K-Y-D-Ala-Y-C-W-F-CONH2 | 14 |
| 15 | Ac-M-Y-C-V-G-K-Y-D-dAla-Y-C-W-F-CONH2 | 15 |
| 16 | Ac-M-Y-C-V-G-K-Y-D-V-Ala-C-W-F-CONH2 | 16 |
| 17 | Ac-M-Y-C-V-G-K-Y-D-V-Y-C-Ala-F-CONH2 | 17 |
| 18 | Ac-hLeu-Y-C-V-G-K-Y-D-V-Y-C-W-F-CONH2 | 18 |
| 19 | Ac-Leu-Y-C-V-G-K-Y-D-V-Y-C-W-F-CONH2 | 19 |
| 20 | Ac-Nle-3FY-C-V-G-K-Y-D-V-Y-C-W-F-CONH2 | 20 |
| 21 | Ac-Nle-3CIY-C-V-G-K-Y-D-V-Y-C-W-F-CONH2 | 21 |
| 22 | Ac-Nle-4COOHPhe-C-V-G-K-Y-D-V-Y-C-W-F-CONH2 | 22 |
| 23 | Ac-Nle-4PyA-C-V-G-K-Y-D-V-Y-C-W-F-CONH2 | 23 |
| 24 | Ac-Nle-Y-C-V-G-K-4COOHPhe-D-V-Y-C-W-F-CONH2 | 24 |
| 25 | Ac-Nle-Y-C-V-G-K-4PyA-D-V-Y-C-W-F-CONH2 | 25 |
| 26 | Ac-Nle-Y-C-V-Sar-K-Y-D-V-Y-C-W-F-CONH2 | 26 |
| 27 | Ac-Nle-Y-C-V-G-K-Y-D-V-Y-C-W-F-Thr-dLys(DOTA)-CONH2 | 27 |
| 28 | Ac-Nle-Y-C-V-G-K-Y-D-V-Y-C-W-F-Thr-dLys(DOTA)-COOH | 28 |
| 29 | Ac-Nle-Y-C-V-G-K-Y-D-V-Y-C-W-F-E-dLys(DOTA)-CONH2 | 29 |
| 30 | Ac-Nle-Y-C-V-G-K-Y-D-V-Y-C-W-F-gE-gE-dLys(DOTA)-CONH2 | 30 |
| 31 | Ac-Nle-Y-C-V-G-K-Y-D-V-Y-C-W-F-gE-dLys(DOTA)-CONH2 | 31 |
| 32 | Ac-Nle-Y-C-V-G-K-Y-D-V-Y-C-W-F-Thr-CONH2 | 32 |
| 33 | Ac-Nle-Y-C-V-G-K-Y-D-V-Y-C-W-F-Thr-COOH | 33 |
| 34 | Ac-Nle-Y-C-V-G-K-Y-D-V-Y-C-W-F-E-CONH2 | 34 |
| 35 | Ac-Nle-Y-C-V-G-K-Y-D-V-Y-C-W-F-gE-CONH2 | 35 |
| 36 | Ac-Nle-Y-C-V-G-K-Y-D-V-Y-C-W-F-gE-gE-CONH2 | 36 |
| 37 | Ac-Ala-Y-C-V-G-K-Y-D-V-Y-C-W-F-CONH2 | 37 |
| 38 | Ac-dAla-Y-C-V-G-K-Y-D-V-Y-C-W-F-CONH2 | 38 |
| 39 | Ac-M-Y-C-dAla-G-K-Y-D-V-Y-C-W-F-CONH2 | 39 |
| 40 | Ac-M-Y-C-V-Sar-K-Y-D-V-Y-C-W-F-CONH2 | 40 |
| 41 | Ac-M-Y-C-V-Ala-K-Y-D-V-Y-C-W-F-CONH2 | 41 |
| 42 | Ac-M-Y-C-V-G-Ala-Y-D-V-Y-C-W-F-CONH2 | 42 |
| 43 | Ac-M-Y-C-V-G-dAla-Y-D-V-Y-C-W-F-CONH2 | 43 |
| 44 | Ac-M-Y-C-V-G-K-Y-D-V-dAla-C-W-F-CONH2 | 44 |
| 45 | Ac-M-Y-C-V-G-K-Y-D-V-Y-C-dAla-F-CONH2 | 45 |
| 46 | Ac-M-Y-C-V-G-K-Y-D-V-Y-C-W-Ala-CONH2 | 46 |
| 47 | Ac-M-Y-C-V-G-K-Y-D-V-Y-C-W-dAla-CONH2 | 47 |
| 48 | Ac-M-Y-dCys-V-G-K-Y-D-V-Y-C-W-F-CONH2 | 48 |
| 49 | Ac-M-Y-C-V-G-K-Y-D-V-Y-dCys-W-F-CONH2 | 49 |
| 50 | Ac-Nle-Y-C-V-G-K-Y-D-V-Y-C-W-F-Lys(DOTA)-CONH2 | 50 |
| 51 | Ac-Nle-Y-C-V-S-K-Y-D-V-Y-C-W-F-COOH | 51 |
| 52 | Ac-Nle-Y-C-V-NetGly-K-Y-D-V-Y-C-W-F-CONH2 | 52 |
| 53 | Ac-Nle-Y-C-V-N(CH2)2COOHGly-K-Y-D-V-Y-C-W-F-CONH2 | 53 |
| 54 | Ac-Nle-Y-C-ChG-G-K-Y-D-V-Y-C-W-F-Thr-dLys(DOTA)-CONH2 | 54 |
| 55 | Ac-Nle-Y-C-V-G-K-Y-D-ChG-Y-C-W-F-Thr-dLys(DOTA)-CONH2 | 55 |
| 56 | Ac-Leu-Y-C-V-G-K-Y-D-V-Y-C-W-F-Thr-dLys(DOTA)-CONH2 | 56 |
| 57 | Ac-Nle-4PyA-C-V-G-K-Y-D-V-Y-C-W-F-Thr-dLys(DOTA)-CONH2 | 57 |
| 58 | Ac-Nie-Y-C-V-G-K-4PyA-D-V-Y-C-W-F-Thr-dLys(DOTA)-CONH2 | 58 |
| 59 | Ac-Nle-Y-C-V-G-K-Y-D-V-Y-C-5FW-F-Thr-CONH2 | 59 |
| 60 | Ac-Nle-Y-C-V-G-K-Y-D-V-Y-C-7NW-F-Thr-CONH2 | 60 |
| 61 | Ac-Nle-Y-C-Tbg-G-K-Y-D-V-Y-C-W-F-Thr-dLys(DOTA)-CONH2 | 61 |
| 62 | Ac-Nle-Y-C-V-G-K-Y-D-Tbg-Y-C-W-F-Thr-dLys(DOTA)-CONH2 | 62 |
| 63 | Ac-Nle-Y-C-V-G-Orn-Y-D-V-Y-C-W-F-Thr-dLys(DOTA)-CONH2 | 63 |
| 64 | Ac-Nle-Y-hC-V-G-K-Y-D-V-Y-hC-W-F-Thr-dLys(DOTA)-CONH2 | 64 |
| 65 | Ac-Nle-Y-C-V-G-Lys(Ac)-Y-D-V-Y-C-W-F-Thr-dLys(DOTA)- | 65 |
| | CONH2 | |
| 66 | Ac-Nle-Y-C-V-G-Nle-Y-D-V-Y-C-W-F-Thr-dLys(DOTA)-CONH2 | 66 |
| 67 | Ac-hLeu-Y-C-V-G-K-Y-D-V-Y-C-W-F-Thr-dLys(DOTA)-CONH2 | 67 |
| 68 | Ac-Nle-Y-C-V-G-K-Y-D-V-Y-C-W-homoPhe-Thr-dLys(DOTA)- | 68 |
| | CONH2 | |
| 69 | Ac-Nle-Y-C-V-G-K-Y-D-V-Y-C-W-4CNPhe-Thr-dLys(DOTA)- | 69 |
| | CONH2 | |
| 70 | Ac-Nle-Y-C-V-G-K-Y-D-V-Y-C-W-3CNPhe-Thr-dLys(DOTA)- | 70 |
| | CONH2 | |
| 71 | Ac-Nle-Y-C-Abu-G-K-Y-D-V-Y-C-W-F-Thr-dLys(DOTA)-CONH2 | 71 |
| 72 | Ac-hLeu-Y-C-Tbg-G-K-Y-D-V-Y-C-W-F-Thr-dLys(DOTA)-CONH2 | 72 |
| 73 | Ac-hLeu-Y-C-V-G-K-Y-D-V-Y-C-5FW-F-Thr-dLys(DOTA)-CONH2 | 73 |
| 74 | Ac-hLeu-Y-C-V-Sar-K-Y-D-V-Y-C-W-F-Thr-dLys(DOTA)-CONH2 | 74 |
| 75 | Ac-hLeu-Y-C-Tbg-Sar-K-Y-D-V-Y-C-W-F-Thr-dLys(DOTA)-CONH2 | 75 |
| 76 | Ac-hLeu-Y-C-Tbg-G-K-Y-D-V-Y-C-5FW-F-Thr-dLys(DOTA)- | 76 |
| | CONH2 | |
| 77 | Ac-hLeu-Y-C-V-Sar-K-Y-D-V-Y-C-5FW-F-Thr-dLys(DOTA)-CONH2 | 77 |
| 78 | Ac-hLeu-Y-C-Tbg-Sar-K-Y-D-V-Y-C-5FW-F-Thr-dLys(DOTA)- | 78 |
| | CONH2 | |
| 79 | Ac-Nle-Y-C-V-G-4PyA-Y-D-V-Y-C-W-F-Thr-dLys(DOTA)-CONH2 | 79 |
| 80 | Ac-Nle-Y-C-V-G-K-Y-N-V-Y-C-W-F-Thr-dLys(DOTA)-CONH2 | 80 |
| 81 | Ac-Nle-Y-C-V-G-K-Y-S-V-Y-C-W-F-Thr-dLys(DOTA)-CONH2 | 81 |
| 82 | Ac-Nle-Y-C-V-G-K-Y-D-V-Y-C-W-ChA-Thr-dLys(DOTA)-CONH2 | 82 |
| 83 | Ac-Nle-Y-C-V-G-K-Y-D-V-Y-C-W-3aminomethylPhe-Thr- | 83 |
| | dLys(DOTA)-CONH2 | |
| 84 | Ac-Nle-Y-C-V-G-K-Y-D-V-Y-C-W-4aminomethylPhe-Thr- | 84 |
| | dLys(DOTA)-CONH2 | |
| 85 | Ac-Nle-Y-C-V-G-Arg-Y-D-V-Y-C-W-F-Thr-dLys(DOTA)-CONH2 | 85 |
| 86 | Ac-Nle-Y-C-V-G-K-Y-D-V-Y-C-W-F-Lys-dLys(DOTA)-CONH2 | 86 |
| 87 | Ac-Nle-Y-C-V-G-K-Y-D-V-Y-C-W-F-Orn-dLys(DOTA)-CONH2 | 87 |
| 88 | Ac-Nle-Y-C-V-G-K-Y-D-V-Y-C-W-F-Dap-dLys(DOTA)-CONH2 | 88 |
| 89 | Ac-Nle-Y-C-V-G-K-Y-D-V-Y-C-W-F-4PipAla-dLys(DOTA)-CONH2 | 89 |
| 90 | Ac-Nle-Y-C-V-G-K-Y-D-V-Y-C-W-F-dLys-dLys(DOTA)-CONH2 | 90 |
| 91 | Ac-Nle-Y-C-V-G-K-Y-D-V-Y-C-W-F-dGlu-dLys(DOTA)-CONH2 | 91 |
| 92 | Ac-Nle-Y-C-V-G-K-Y-D-V-Y-C-W-F-Ser-dLys(DOTA)-CONH2 | 92 |
| 93 | Ac-Nle-Y-C-V-G-K-Y-D-V-Y-C-W-F-alloThr-dLys(DOTA)-CONH2 | 93 |
| 94 | DOTA-PEG8-Nle-Y-C-V-G-K-Y-D-V-Y-C-W-F-Thr-CONH2 | 94 |
| 95 | Ac-Nle-Y-C-NMeVal-G-K-Y-D-V-Y-C-W-F-Thr-dLys(DOTA)- | 95 |
| | CONH2 | |
| 96 | Ac-Nle-Y-C-V-G-K-Y-D-Abu-Y-C-W-F-Thr-dLys(DOTA)-CONH2 | 96 |
| 97 | Ac-Nle-Y-C-V-G-K-Y-D-NMeVal-Y-C-W-F-Thr-dLys(DOTA)- | 97 |
| | CONH2 | |
| 98 | Ac-hLeu-Y-C-V-G-K-Y-D-V-Y-C-W-Lys(DOTA)-Thr-CONH2 | 98 |
| 99 | Ac-hLeu-Y-C-V-G-K-Y-D-V-Y-C-Lys(DOTA)-F-Thr-CONH2 | 99 |
| 100 | Ac-hLeu-Y-C-V-dLys(DOTA)-K-Y-D-V-Y-C-W-F-Thr-CONH2 | 100 |
| 101 | Ac-Nle-Y-C-V-G-K-Y-D-V-HomoArg-C-W-F-Thr-dLys(DOTA)- | 101 |
| | CONH2 | |
| 102 | Ac-Nle-Y-C-V-G-K-Y-HomoGlu-V-Y-C-W-F-Thr-dLys(DOTA)- | 102 |
| | COOH | |
| 103 | Ac-Nle-Y-C-V-G-HomoGlu-Y-D-V-Y-C-W-F-Thr-dLys(DOTA)- | 103 |
| | COOH | |
| 104 | Ac-Nle-Y-C-V-HomoGlu-K-Y-D-V-Y-C-W-F-Thr-dLys(DOTA)- | 104 |
| | COOH | |
| 105 | Ac-Nle-Y-C-HomoGlu-G-K-Y-D-V-Y-C-W-F-Thr-dLys(DOTA)- | 105 |
| | COOH | |
| 106 | Ac-Nle-HomoGlu-C-V-G-K-Y-D-V-Y-C-W-F-Thr-dLys(DOTA)- | 106 |
| | COOH | |
| 107 | Ac-HomoGlu-Y-C-V-G-K-Y-D-V-Y-C-W-F-Thr-dLys(DOTA)-COOH | 107 |
| 108 | Ac-Nle-Y-C-V-G-K-Y-D-V-Y-C-W-HomoArg-Thr-dLys(DOTA)- | 108 |
| | CONH2 | |
| 109 | Ac-Nle-Y-C-V-G-K-Y-D-V-Y-C-HomoArg-F-Thr-dLys(DOTA)- | 109 |
| | CONH2 | |
| 110 | Ac-Nle-HomoArg-C-V-G-K-Y-D-V-Y-C-W-F-Thr-dLys(DOTA)- | 110 |
| | CONH2 | |
| 111 | Ac-HomoArg-Y-C-V-G-K-Y-D-V-Y-C-W-F-Thr-dLys(DOTA)- | 111 |
| | CONH2 | |
| 112 | Ac-Nle-Y-C-V-G-K-Y-D-V-Y-C-W-HomoGlu-Thr-dLys(DOTA)- | 112 |
| | COOH | |
| 113 | Ac-Nle-Y-C-V-G-K-Y-D-V-Y-C-HomoGlu-F-Thr-dLys(DOTA)- | 113 |
| | COOH | |
| 114 | Ac-Nle-Y-C-V-G-K-Y-D-V-HomoGlu-C-W-F-Thr-dLys(DOTA)- | 114 |
| | COOH | |
| 115 | Ac-Nle-Y-C-V-G-K-Y-D-HomoGlu-Y-C-W-F-Thr-dLys(DOTA)- | 115 |
| | COOH | |
| 116 | Ac-Nle-Y-C-V-G-K-HomoGlu-D-V-Y-C-W-F-Thr-dLys(DOTA)- | 116 |
| | COOH | |
| 117 | Ac-hLeu-Y-Pen-V-G-K-Y-D-V-Y-C-W-F-Thr-dLys(DOTA)-CONH2 | 117 |
| 118 | Ac-hLeu-Y-C-V-G-K-Y-D-V-Y-Pen-W-F-Thr-dLys(DOTA)-CONH2 | 118 |
| 119 | Ac-hLeu-Y-C-Cpg-G-K-Y-D-V-Y-C-W-F-Thr-dLys(DOTA)-CONH2 | 119 |
| 120 | Ac-hLeu-Y-C-alloThr-G-K-Y-D-V-Y-C-W-F-Thr-dLys(DOTA)- | 120 |
| | CONH2 | |
| 121 | Ac-hLeu-Y-C-allolle-G-K-Y-D-V-Y-C-W-F-Thr-dLys(DOTA)-CONH2 | 121 |
| 122 | Ac-hLeu-Y-C-hydroVal-G-K-Y-D-V-Y-C-W-F-Thr-dLys(DOTA)- | 122 |
| | CONH2 | |
| 123 | Ac-hLeu-Y-C-V-G-K-Y-D-Cpg-Y-C-W-F-Thr-dLys(DOTA)-CONH2 | 123 |
| 124 | Ac-hLeu-Y-C-V-G-K-Y-D-Aib-Y-C-W-F-Thr-dLys(DOTA)-CONH2 | 124 |
| 125 | Ac-hLeu-Y-C-V-G-K-Y-D-alloThr-Y-C-W-F-Thr-dLys(DOTA)- | 125 |
| | CONH2 | |
| 126 | Ac-hLeu-Y-C-V-G-K-Y-D-allolle-Y-C-W-F-Thr-dLys(DOTA)-CONH2 | 126 |
| 127 | Ac-hLeu-Y-C-V-G-K-Y-D-hydroVal-Y-C-W-F-Thr-dLys(DOTA)- | 127 |
| | CONH2 | |
| 128 | Ac-Nle-Y-C-V-G-K-Y-D-V-Y-C-1Nal-F-Thr-dLys(DOTA)-CONH2 | 128 |
| 129 | Ac-Nle-Y-C-V-G-K-Y-D-V-Y-C-2Nal-F-Thr-dLys(DOTA)-CONH2 | 129 |
| 130 | Ac-Nle-Y-C(CH2)-V-G-K-Y-D-V-Y-C-W-F-Thr-dLys(DOTA)-CONH2 | 130 |
| 131 | Ac-Nle-Y-C-V-G-K-Y-D-V-Y-C-W-F-Dab-dLys(DOTA)-CONH2 | 13 |
| 132 | Ac-Nle-Y-C-V-G-K-Y-D-V-Y-C-Trp(Me)-F-Thr-dLys(DOTA)-CONH2 | 132 |
| 133 | Ac-Nle-Y-C-V-G-K-Y-D-V-Y-C-5FW-F-Thr-dLys(DOTA)-CONH2 | 133 |
| 134 | Ac-Nle-Y-C-V-G-K-Y-D-V-Y-C-7NW-F-Thr-dLys(DOTA)-CONH2 | 134 |
| 135 | Ac-hLeu-Y-C-V-G-K-PipAla-D-V-Y-C-W-F-Thr-dLys(DOTA)- | 135 |
| | CONH2 | |
| 136 | Ac-hLeu-Y-C-V-G-K-AEF(DOTA)-D-V-Y-C-W-F-Thr-dAla-CONH2 | 136 |
| 137 | Ac-hLeu-AEF(DOTA)-C-V-G-K-Y-D-V-Y-C-W-F-Thr-dAla-CONH2 | 137 |
| 138 | Ac-hLeu-Y-C-Tbg-G-Orn-Y-D-Chg-Y-C-W-F-Thr-dLys(DOTA)- | 138 |
| | CONH2 | |
| 139 | Ac-hLeu-Y-C-Tbg-G-K-4COOHPhe-D-V-Y-C-W-F-Thr- | 139 |
| | dLys(DOTA)-CONH2 | |
| 140 | Ac-hLeu-Y-C-Tbg-Sar-K-4COOHPhe-D-V-Y-C-W-F-Thr- | 140 |
| | dLys(DOTA)-CONH2 | |
| 141 | Ac-hLeu-Y-C-V-G-K-Y-D-V-Y-C-W-F-Pro-dLys(DOTA)-CONH2 | 141 |
| 142 | Ac-hLeu-Y-C-V-G-K-Y-D-V-Lys(DOTA)-C-W-F-Thr-CONH2 | 142 |
| 143 | Ac-hLeu-Y-C-V-G-K-Y-D-Lys(DOTA)-Y-C-W-F-Thr-CONH2 | 143 |
| 144 | Ac-hLeu-Y-C-V-G-K-Lys(DOTA)-D-V-Y-C-W-F-Thr-CONH2 | 144 |
| 145 | Ac-hLeu-Y-C-V-G-Lys(DOTA)-Y-D-V-Y-C-W-F-Thr-CONH2 | 145 |
| 146 | Ac-Nle-Y-C-allolle-G-K-Y-D-V-Y-C-W-F-Thr-dLys(DOTA)-CONH2 | 146 |
| 147 | Ac-hLeu-Y-C-V-G-K-Y-D-V-Y-C-5FW-F-aMeSer-dLys(DOTA)- | 147 |
| | CONH2 | |
| 148 | Ac-hLeu-Y-C-V-G-K-Y-D-V-Y-C-5FW-F-NMeSer-dLys(DOTA)- | 148 |
| | CONH2 | |
| 149 | Ac-hLeu-Y-C-V-G-K-Y-D-V-Y-C-W-F-hydroxyVal-dLys(DOTA)- | 149 |
| | CONH2 | |
| 150 | Ac-hLeu-Y-C-V-G-K-Y-D-V-Y-C-Trp(Me)-F-Thr-dLys(DOTA)- | 150 |
| | CONH2 | |
| 151 | Ac-hLeu-Y-C-V-G-K-Y-D-V-3FY-C-W-F-Thr-dLys(DOTA)-CONH2 | 151 |
| 152 | Ac-hLeu-Y-C-Tbg-G-K-4PyA-D-ChG-Y-C-W-F-Thr-dLys(DOTA)- | 152 |
| | CONH2 | |
| 153 | Ac-hLeu-Y-C-V-G-K-4PyA-D-ChG-Y-C-W-F-Thr-dLys(DOTA)- | 153 |
| | CONH2 | |
| 154 | Ac-hLeu-Y-C-V-G-PipAla-Y-D-V-Y-C-W-F-Thr-dLys(DOTA)- | 154 |
| | CONH2 | |
| 155 | Ac-hLeu-Y-C-V-G-K-4PyA-D-V-Y-C-W-F-Thr-dLys(DOTA)-CONH2 | 155 |
| 156 | Ac-hLeu-Y-C-V-G-K-Y-D-V-Y-C-7NW-F-Thr-dLys(DOTA)-CONH2 | 156 |
| 157 | Ac-hLeu-Y-C-Tbg-G-K-4PyA-D-V-Y-C-W-F-Thr-dLys(DOTA)- | 157 |
| | CONH2 | |
| 158 | Ac-hLeu-Y-C-V-G-K-Y-D-V-Y-C-1Nal-F-Thr-dLys(DOTA)-CONH2 | 158 |
| 159 | Ac-hLeu-Y-C-V-G-K-Y-D-V-Y-C-2Nal-F-Thr-dLys(DOTA)-CONH2 | 159 |
| 160 | Ac-hLeu-Y-C-V-G-K-Y-D-V-Y-C-5FW-F-Pro-dLys(DOTA)-CONH2 | 160 |
| 161 | Ac-hLeu-Y-C-V-G-K-Y-D-V-Y-C-5FW-F-4OHPro-dLys(DOTA)- | 161 |
| | CONH2 | |
| 162 | Ac-hLeu-Y-C-V-G-K-Y-D-V-Y-C-5FW-F-alloThr-dLys(DOTA)- | 162 |
| | CONH2 | |
| 163 | Ac-hLeu-Y-C-V-G-K-Y-D-V-Y-C-5FW-F-dThr-dLys(DOTA)-CONH2 | 163 |
| 164 | Ac-hLeu-Y-C-V-G-K-Y-D-V-Y-C-5FW-2FPhe-Thr-dLys(DOTA)- | 164 |
| | CONH2 | |
| 165 | Ac-hLeu-Y-C-V-G-K-Y-D-V-Y-C-5FW-2MePhe-Thr-dLys(DOTA)- | 165 |
| | CONH2 | |
| 166 | Ac-hLeu-Y-C-V-G-K-Y-D-V-Y-C-5FW-4COOHPhe-Thr- | 166 |
| | dLys(DOTA)-CONH2 | |
| 167 | Ac-hLeu-Y-C-V-G-K-hTyr-D-V-Y-C-5FW-F-Thr-dLys(DOTA)- | 167 |
| | CONH2 | |
| 168 | Ac-hLeu-Y-C-V-G-K-Y-D-V-Y-C-5FW-aMePhe-Thr-dLys(DOTA)- | 168 |
| | CONH2 | |
| 169 | Ac-hLeu-Y-C-PipAla-G-Lys(DOTA)-Y-D-V-Y-C-5FW-F-Thr-CONH2 | 169 |
| 170 | Ac-hLeu-Y-C-V-G-K-Y-D-V-3PyA-C-5FW-F-Thr-dLys(DOTA)- | 170 |
| | CONH2 | |
| 171 | Ac-hLeu-Y-C-V-G-K-Y-D-V-OMeTyr-C-5FW-F-Thr-dLys(DOTA)- | 171 |
| | CONH2 | |
| 172 | Ac-hLeu-Y-C-V-G-K-Y-D-V-4FPhe-C-5FW-F-Thr-dLys(DOTA)- | 172 |
| | CONH2 | |
| 173 | Ac-hLeu-Y-C-V-G-K-Y-D-V-Y-C-5FW-3FPhe-Thr-dLys(DOTA)- | 173 |
| | CONH2 | |
| 174 | Ac-hLeu-Y-C-tBuAla-G-K-Y-D-V-Y-C-5FW-F-Thr-dLys(DOTA)- | 174 |
| | CONH2 | |
| 175 | Ac-hLeu-Y-C-V-G-K-Y-D-V-Y-C-5FW-aMeNle-Thr-dLys(DOTA)- | 175 |
| | CONH2 | |
| 176 | Ac-hLeu-Y-C-V-G-K-Y-D-V-Y-C-5FW-NMeNle-Thr-dLys(DOTA)- | 176 |
| | CONH2 | |
| 177 | Ac-hLeu-Y-C-Tbg-G-K-Y-D-V-Y-C-aMeW-F-Thr-dLys(DOTA)- | 177 |
| | CONH2 | |
| 178 | Ac-hLeu-Y-C-V-G-K-alloThr-D-V-Y-C-5FW-F-Thr-dLys(DOTA)- | 178 |
| | CONH2 | |
| 179 | Ac-hLeu-Y-C-Tbg-G-K-Y-D-V-Y-C-1Nal-F-Thr-dLys(DOTA)- | 179 |
| | CONH2 | |
| 180 | Ac-hLeu-Y-C-Tbg-G-K-Y-D-Chg-Y-C-1Nal-F-Thr-dLys(DOTA)- | 180 |
| | CONH2 | |
| 181 | Ac-hLeu-Y-C-Tbg-G-K-Y-D-Chg-Y-C-5FW-F-Thr-dLys(DOTA)- | 181 |
| | CONH2 | |
| 182 | Ac-hLeu-Y-C-V-G-K-MAF(DOTA)-D-V-Y-C-5FW-F-Thr-CONH2 | 182 |
| 183 | Ac-hLeu-Y-C-V-G-K-Y-D-V-MAF(DOTA)-C-5FW-F-Thr-CONH2 | 183 |
| 184 | Ac-hLeu-Y-C-Nva-G-K-Y-D-V-Y-C-5FW-F-Thr-dLys(DOTA)- | 184 |
| | CONH2 | |
| 185 | Ac-hLeu-Y-C-V-G-K-Y-D-Nva-Y-C-5FW-F-Thr-dLys(DOTA)- | 185 |
| | CONH2 | |
| 186 | Ac-hLeu-Y-C-V-G-K-Y-D-Nle-Y-C-5FW-F-Thr-dLys(DOTA)- | 186 |
| | CONH2 | |
| 187 | Ac-hLeu-Y-C-V-G-K-Y-D-V-Y-dCys(CH2)-5FW-F-Thr-dLys(DOTA)- | 187 |
| | CONH2 | |
| 188 | Ac-hLeu-Y-dCys(CH2)-V-G-K-Y-D-V-Y-dCys-5FW-F-Thr- | 188 |
| | dLys(DOTA)-CONH2 | |
| 189 | Ac-hLeu-Y-C(CH2)-V-G-K-Y-D-V-Y-C-5FW-F-Thr-dLys(DOTA)- | 189 |
| | CONH2 | |
| 190 | Ac-hLeu-Y-C-V-G-K-4PyA-D-ChG-Y-C-5FW-F-Thr-dLys(DOTA)- | 190 |
| | CONH2 | |
| 191 | Ac-hLeu-Y-C-V-G-K-Y-D-V-AEF(DOTA)-C-5FW-F-Thr-CONH2 | 191 |
| 192 | Ac-hLeu-4FPhe-C-V-G-K-Y-D-V-Y-C-5FW-F-Thr-dLys(DOTA)- | 192 |
| | CONH2 | |
| 193 | Ac-hLeu-OMeTyr-C-V-G-K-Y-D-V-Y-C-5FW-F-Thr-dLys(DOTA)- | 193 |
| | CONH2 | |
| 194 | Ac-hLeu-hTyr-C-V-G-K-Y-D-V-Y-C-5FW-F-Thr-dLys(DOTA)- | 194 |
| | CONH2 | |
| 195 | Ac-hLeu-aMeTyr-C-V-G-K-Y-D-V-Y-C-5FW-F-Thr-dLys(DOTA)- | 195 |
| | CONH2 | |
| 196 | Ac-hLeu-aMeNle-C-V-G-K-Y-D-V-Y-C-5FW-F-Thr-dLys(DOTA)- | 196 |
| | CONH2 | |
| 197 | Ac-hLeu-NMeNle-C-V-G-K-Y-D-V-Y-C-5FW-F-Thr-dLys(DOTA)- | 197 |
| | CONH2 | |
| 198 | Ac-hLeu-Y-C-V-G-K-T-D-V-Y-C-5FW-F-Thr-dLys(DOTA)-CONH2 | 198 |
| 199 | Ac-hLeu-Y-C-V-G-K-hSer-D-V-Y-C-5FW-F-Thr-dLys(DOTA)- | 199 |
| | CONH2 | |
| 200 | Ac-hLeu-Y-C-V-G-K-ThpA-D-V-Y-C-5FW-F-Thr-dLys(DOTA)- | 200 |
| | CONH2 | |
| 201 | Ac-hLeu-Y-C-V-G-Lys(DOTA)-CHA-D-V-Y-C-5FW-F-hydroxyVal- | 201 |
| | CONH2 | |
| 202 | Ac-hLeu-Y-C-V-G-Lys(DOTA)-PipAla-D-V-Y-C-5FW-F-hydroxyVal- | 202 |
| | CONH2 | |
| 203 | Ac-hLeu-Y-C-Ile-G-Lys(DOTA)-PipAla-D-V-Y-C-5FW-F- | 203 |
| | hydroxyVal-CONH2 | |
| 204 | Ac-hLeu-Thr-C-V-G-PipAla-Y-D-V-Y-C-5FW-F-NMeSer- | 204 |
| | dLys(DOTA)-CONH2 | |
| 205 | Ac-hLeu-Y-C-V-G-K-Y-D-V-Y-C-5FW-NMePhe-Thr-dLys(DOTA)- | 205 |
| | CONH2 | |
| 206 | Ac-hLeu-Y-C-V-G-K-ChA-D-V-Y-C-5FW-F-Thr-dLys(DOTA)- | 206 |
| | CONH2 | |
| 207 | Ac-hLeu-ThpA-C-V-G-PipAla-Y-D-V-Y-C-5FW-F-NMeSer- | 207 |
| | dLys(DOTA)-CONH2 | |
| 208 | Ac-hLeu-Tba-C-V-G-PipAla-Y-D-V-Y-C-5FW-F-NMeSer- | 208 |
| | dLys(DOTA)-CONH2 | |
| 209 | Ac-hLeu-Nle-C-V-G-PipAla-Y-D-V-Y-C-5FW-F-NMeSer- | 209 |
| | dLys(DOTA)-CONH2 | |
| 210 | Ac-hLeu-allo Thr-C-V-G-PipAla-Y-D-V-Y-C-5FW-F-NMeSer- | 210 |
| | dLys(DOTA)-CONH2 | |
| 211 | Ac-hLeu-hyVal-C-V-G-PipAla-Y-D-V-Y-C-5FW-F-NMeSer- | 211 |
| | dLys(DOTA)-CONH2 | |
| 212 | Ac-hLeu-Chg-C-V-G-PipAla-Y-D-V-Y-C-5FW-F-NMeSer- | 212 |
| | dLys(DOTA)-CONH2 | |
| 213 | Ac-hLeu-ThpG-C-V-G-PipAla-Y-D-V-Y-C-5FW-F-NMeSer- | 213 |
| | dLys(DOTA)-CONH2 | |
| 214 | Ac-hLeu-AEF(DOTA)-C-V-G-K-Y-D-V-Y-C-5FW-F-Thr-CONH2 | 214 |
| 215 | Ac-hLeu-Y-C-V-G-K-Y-D-ChG-Y-C-5FW-F-Thr-dLys(DOTA)- | 215 |
| | CONH2 | |
| 216 | Ac-hLeu-Y-C-Tbg-G-K-Y-D-ChG-Y-C-W-F-Thr-dLys(DOTA)- | 216 |
| | CONH2 | |
| 217 | Ac-hLeu-Y-C-Nle-G-K-Y-D-V-Y-C-5FW-F-Thr-dLys(DOTA)- | 217 |
| | CONH2 | |
| 218 | Ac-hLeu-MAF(DOTA)-C-V-G-K-Y-D-V-Y-C-5FW-F-Thr-CONH2 | 218 |
| 219 | Ac-hLeu-Y-C-Tbg-G-K-4PyA-D-V-Y-C-1Nal-F-NMeSer- | 219 |
| | dLys(DOTA)-CONH2 | |
| 220 | Ac-hLeu-Y-C-Tbg-G-K-4PyA-D-V-Cha-C-1Nal-F-NMeSer- | 220 |
| | dLys(DOTA)-CONH2 | |
| 221 | Ac-hLeu-Y-C-Tbg-G-K-4PyA-D-V-CyanoButric-C-1Nal-F-NMeSer- | 221 |
| | dLys(DOTA)-CONH2 | |
| 222 | Ac-hLeu-Y-C-Tbg-G-K-4PyA-D-V-Thr-C-1Nal-F-NMeSer- | 222 |
| | dLys(DOTA)-CONH2 | |
| 223 | Ac-hLeu-Y-C-Tbg-G-K-4PyA-D-V-ThpA-C-1Nal-F-NMeSer- | 223 |
| | dLys(DOTA)-CONH2 | |
| 224 | Ac-hLeu-Y-C-Tbg-G-K-4PyA-D-V-Tba-C-1Nal-F-NMeSer- | 224 |
| | dLys(DOTA)-CONH2 | |
| 225 | Ac-hLeu-Y-C-Tbg-G-K-4PyA-D-V-Nle-C-1Nal-F-NMeSer- | 225 |
| | dLys(DOTA)-CONH2 | |
| 226 | Ac-hLeu-Y-C-Tbg-G-K-4PyA-D-V-alloThr-C-1Nal-F-NMeSer- | 226 |
| | dLys(DOTA)-CONH2 | |
| 227 | Ac-hLeu-Y-C-V-G-PipAla-CHA-D-V-Y-C-5FW-F-hydroxyVal- | 227 |
| | dLys(DOTA)-CONH2 | |
| 228 | Ac-hLeu-Y-C-V-G-Lys(DOTA)-4PyA-D-V-Y-C-5FW-F-NMeSer- | 228 |
| | CONH2 | |
| 229 | Ac-hLeu-Y-C-V-G-Lys(DOTA)-4PyA-D-V-Y-C-5FW-F-hydroxyVal- | 229 |
| | CONH2 | |
| 230 | Ac-hLeu-Y-C-Ile-G-Lys(DOTA)-4PyA-D-V-Y-C-5FW-F-hydroxyVal- | 230 |
| | CONH2 | |
| 231 | Ac-hLeu-Y-C-Tbg-G-K-4PyA-D-V-hyVal-C-1Nal-F-NMeSer- | 231 |
| | dLys(DOTA)-CONH2 | |
| 232 | Ac-hLeu-Y-C-Ile-G-Lys(DOTA)-CHA-D-V-Y-C-5FW-F-NMeSer- | 232 |
| | CONH2 | |
| 233 | Ac-hLeu-Y-C-V-G-Lys(DOTA)-CHA-D-V-Y-C-5FW-F-NMeSer- | 233 |
| | CONH2 | |
| 234 | Ac-hLeu-Cba-C-V-G-PipAla-Y-D-V-Y-C-5FW-F-NMeSer- | 234 |
| | dLys(DOTA)-CONH2 | |
| 235 | Ac-hLeu-Y-C-Tbg-G-K-Y-D-V-Y-C-5ClTrp-F-Thr-dLys(DOTA)- | 235 |
| | CONH2 | |
| 236 | Ac-hLeu-Y-C-V-G-PipAla-CHA-D-V-Y-C-5FW-F-NMeSer- | 236 |
| | dLys(DOTA)-CONH2 | |
| 237 | Ac-hLeu-Y-NMeCys-V-G-K-Y-D-V-Y-C-5FW-F-Thr-dLys(DOTA)- | 237 |
| | CONH2 | |
| 238 | Ac-hLeu-Ala-C(CH2)-V-G-PipAla-Y-D-V-Y-C-5FW-F-NMeSer- | 238 |
| | dLys(DOTA)-CONH2 | |
| 239 | Ac-hLeu-Y-C(CH2)-V-G-PipAla-Y-D-V-Y-C-5FW-F-NMeSer- | 239 |
| | dLys(DOTA)-CONH2 | |
| 240 | Ac-hLeu-Y-C-V-G-K-4PyA-D-V-Y-C-5FW-F-Thr-dLys(DOTA)- | 240 |
| | CONH2 | |
| 241 | Ac-hLeu-Y-Pra-V-G-K-Y-D-V-Y-DAPN3-5FW-F-Thr-dLys(DOTA)- | 241 |
| | CONH2 | |
| 242 | Ac-hLeu-Y-Pra-V-G-K-Y-D-V-Y-DAPN3-5FW-F-Thr-dLys(DOTA)- | 242 |
| | CONH2 | |
| 243 | Ac-hLeu-Y-C-Tbg-G-K-4PyA-D-V-Chg-C-1Nal-F-NMeSer- | 243 |
| | dLys(DOTA)-CONH2 | |
| 244 | Ac-hLeu-Y-C-Tbg-G-K-Y-D-V-Y-C-1Nal-F-Pro-dLys(DOTA)- | 244 |
| | CONH2 | |
| 245 | Ac-hLeu-Y-C-V-G-K-Y-D-V-Y-C-1Nal-F-Pro-dLys(DOTA)-CONH2 | 245 |
| 246 | Ac-hLeu-Y-C-Tbg-G-K-Y-D-V-Y-C-1Nal-F-aMePro-dLys(DOTA)- | 246 |
| | CONH2 | |
| 247 | Ac-hLeu-Y-C-V-G-K-Y-D-V-Y-C-1Nal-F-aMePro-dLys(DOTA)- | 247 |
| | CONH2 | |
| 248 | Ac-hLeu-Y-C-Tbg-G-K-Y-D-V-Y-C-5FW-F-Pro-dLys(DOTA)- | 248 |
| | CONH2 | |
| 249 | Ac-hLeu-Y-C-V-G-Lys(DOTA)-4PyA-D-V-OMeTyr-C-5FW-F-Pro- | 249 |
| | CONH2 | |
| 250 | Ac-hLeu-Y-C-Tbg-G-Lys(DOTA)-4PyA-D-V-Y-C-1Nal-F-(cisHyp)- | 250 |
| | CONH2 | |
| 251 | Ac-hLeu-Y-C-Ile-G-Lys(DOTA)-4PyA-D-V-Y-C-5FW-F-(cisHyp)- | 251 |
| | CONH2 | |
| 252 | Ac-hLeu-Y-C-Tbg-G-Lys(gE-DOTA)-4PyA-D-V-Y-C-1Nal-F-Pro- | 252 |
| | CONH2 | |
| 253 | Ac-hLeu-Y-C-Tbg-G-Lys(gE-gE-DOTA)-4PyA-D-V-Y-C-1Nal-F- | 253 |
| | Pro-CONH2 | |
| 254 | Ac-hLeu-Y-C-Tbg-G-K-MAF(gE-DOTA)-D-V-Y-C-1Nal-F-Pro- | 254 |
| | CONH2 | |
| 255 | Ac-hLeu-Y-C-Tbg-G-K-MAF(gE-gE-DOTA)-D-V-Y-C-1Nal-F-Pro- | 255 |
| | CONH2 | |
| 256 | Ac-hLeu-Y-C-Tbg-G-Lys(PEG4-DOTA)-4PyA-D-V-Y-C-1Nal-F-Pro- | 256 |
| | CONH2 | |
| 257 | Ac-hLeu-Y-C-Tbg-G-K-MAF(PEG4-DOTA)-D-V-Y-C-1Nal-F-Pro- | 257 |
| | CONH2 | |
| 258 | Ac-hLeu-Y-C-Tbg-G-K-MAF(gE-gE-gE-DOTA)-D-V-Y-C-1Nal-F- | 258 |
| | Pro-CONH2 | |
| 259 | Ac-hLeu-Y-C-Tbg-G-K-ThpA-D-V-Y-C-1Nal-F-Pro-dLys(PEG4- | 259 |
| | DOTA)-CONH2 | |
| 260 | Ac-hLeu-Y-C-Ile-G-Lys(DOTA)-4PyA-D-V-Y-C-5FW-F-Pro-CONH2 | 260 |
| 261 | Ac-hLeu-Y-C-Ile-G-Lys(DOTA)-ThpA-D-V-Y-C-5FW-F-Pro-CONH2 | 261 |
| 262 | Ac-hLeu-Y-C-V-G-K-MAF(DOTA)-D-V-Y-C-5FW-F-Pro-COOH | 262 |
| 263 | Ac-hLeu-Y-C-Tbg-G-Lys(DOTA)-4PyA-D-V-Y-C-1Nal-F-cis4FPro- | 263 |
| | CONH2 | |
| 264 | Ac-hLeu-Y-C-Tbg-G-Lys(DOTA)-4PyA-D-V-Y-C-1Nal-F- | 264 |
| | trans4FPro-CONH2 | |
| 265 | Ac-hLeu-Y-C-Tbg-G-Lys(DOTA)-4PyA-D-V-Y-C-1Nal-F-Pip acid- | 265 |
| | CONH2 | |
| 266 | Ac-hLeu-Y-C-NMelle-G-Lys(DOTA)-4PyA-D-V-Y-C-5FW-F-Pro- | 266 |
| | CONH2 | |
| 267 | Ac-hLeu-Y-C-lle-G-Lys(DOTA)-4PyA-D-V-Y-C-5FW-F-transHyp- | 267 |
| | CONH2 | |
| 268 | Ac-hLeu-Y-C-Ile-G-Lys(DOTA)-4PyA-D-V-Y-C-5FW-F-cis4FPro- | 268 |
| | CONH2 | |
| 269 | Ac-hLeu-Y-C-Ile-G-Lys(DOTA)-4PyA-D-V-Y-C-5FW-F-Pip acid- | 269 |
| | CONH2 | |
| 271 | Ac-hLeu-Y-C-V-G-K-ThpA-D-V-Y-C-5FW-F-Pro-dLys(DOTA)- | 270 |
| | CONH2 | |
| 272 | DOTA-gE-gE-gE-hLeu-4FPhe-C-Tbg-G-PipAla-ThpA-D-V-Y-C- | 271 |
| | 1Nal-F-Pro-CONH2 | |
| 273 | Ac-hLeu-Y-C-Tbg-G-Lys(DOTA)-ThpA-D-V-Y-C-5FW-F-Pro- | 272 |
| | CONH2 | |
| 274 | Ac-hLeu-Y-C-Tbg-G-K-ThpA-D-V-Y-C-1Nal-F-Pro-dLys(gE- | 273 |
| | DOTA)-CONH2 | |
| 275 | Ac-hLeu-Y-C-Tbg-G-K-ThpA-D-V-Y-C-1Nal-F-Pro-dLys(gE-gE- | 274 |
| | DOTA)-CONH2 | |
| 276 | Ac-hLeu-Y-C-Tbg-G-K-ThpA-D-V-Y-C-1Nal-F-Pro-dLys(gE-gE-gE- | 275 |
| | DOTA)-CONH2 | |
| 277 | Ac-hLeu-Y-C-Tbg-G-PipAla-CHA-D-V-Y-C-1Nal-F-Pro-dLys(gE- | 276 |
| | DOTA)-CONH2 | |
| 278 | Ac-hLeu-Y-C-Tbg-G-PipAla-CHA-D-V-Y-C-1Nal-F-Pro-dLys(gE- | 277 |
| | gE-DOTA)-CONH2 | |
| 279 | Ac-hLeu-Y-C-Tbg-G-K-MAF(PEG8-DOTA)-D-V-Y-C-1Nal-F-Pro- | 278 |
| | CONH2 | |
| 280 | Ac-hLeu-Y-C-Tbg-G-K-ThpA-D-V-Y-C-1Nal-F-Pro-dLys(PEG8- | 279 |
| | DOTA)-CONH2 | |
| 281 | Ac-hLeu-Y-C-Tbg-G-PipAla-CHA-D-V-Y-C-1Nal-F-Pro-dLys(PEG8- | 280 |
| | DOTA)-CONH2 | |
| 282 | Ac-hLeu-Y-C-V-G-K-ThpA-D-V-Y-C-5FW-F-Pro-dLys(PEG4- | 281 |
| | DOTA)-CONH2 | |
| 283 | DOTA-PEG4-hLeu-4FPhe-C-Tbg-G-PipAla-ThpA-D-V-Y-C-1Nal-F- | 282 |
| | Pro-CONH2 | |
| 284 | DOTA-gE-hLeu-7AzaW-C-Tbg-G-PipAla-ThpA-D-V-Y-C-1Nal-F- | 283 |
| | Pro-CONH2 | |
| 285 | DOTA-PEG8-hLeu-Y-C-V-G-PipAla-4COOHPhe-D-V-Y-C-5FW-F- | 284 |
| | Pro-CONH2 | |
| 286 | Ac-hLeu-Y-C-V-G-PipAla-4COOHPhe-D-V-Y-C-5FW-F-Pro- | 285 |
| | dLys(DOTA)-CONH2 | |
| 287 | Ac-hLeu-Y-C-V-G-K-4COOHPhe-D-V-Nle-C-5FW-F-Pro- | 286 |
| | dLys(DOTA)-CONH2 | |
| 288 | Ac-hLeu-Y-C-V-G-K-ThpA-D-V-Y-C-5FW-F-Pro-dLys(DOTA)- | 287 |
| | COOH | |
| 289 | Ac-hLeu-Y-C-V-G-Lys(DOTA)-4COOHPhe-D-V-Y-C-5FW-F-Pro- | 288 |
| | CONH2 | |
| 290 | Ac-hLeu-Y-C-V-G-Lys(DOTA)-4COOHPhe-D-V-Y-C-5FW-F-Pro- | 289 |
| | COOH | |
| 291 | Ac-hLeu-Y-C-V-G-K-4COOHPhe-D-V-Y-C-5FW-F-Pro- | 290 |
| | dLys(DOTA)-CONH2 | |
| 292 | Ac-hLeu-Y-C-V-G-Lys(DOTA)-ThpA-D-V-Y-C-5FW-F-Pro-CONH2 | 291 |
| 293 | DOTA-PEG8-hLeu-Y-C-V-G-K-4COOHPhe-D-V-Nle-C-5FW-F-Pro- | 292 |
| | CONH2 | |
| 294 | DOTA-PEG8-hLeu-Y-C-V-G-K-4COOHPhe-D-V-Y-C-5FW-F-Pro- | 293 |
| | CONH2 | |
| 295 | Ac-hLeu-Y-C-V-G-K-AEF(DOTA)-D-V-Y-C-5FW-F-Thr-CONH2 | 294 |
| 297 | Ac-hLeu-Y-C(CH2)-Tbg-G-K-4PyA-D-V-Y-C-1Nal-F-NMeSer- | 295 |
| | dLys(DOTA)-CONH2 | |
| 298 | Ac-hLeu-Ala-C(CH2)-Tbg-G-K-4PyA-D-V-Y-C-1Nal-F-NMeSer- | 296 |
| | dLys(DOTA)-CONH2 | |
| 299 | Ac-hLeu-Y-C(CH2)-Tbg-G-K-Ala-D-V-Y-C-1Nal-F-NMeSer- | 297 |
| | dLys(DOTA)-CONH2 | |
| 300 | Ac-hLeu-Y-C(CH2)-Tbg-G-K-4PyA-Ala-V-Y-C-1Nal-F-NMeSer- | 298 |
| | dLys(DOTA)-CONH2 | |
| 301 | Ac-hLeu-Y-C-V-G-PipAla-AEF(DOTA)-D-V-Y-C-5FW-F-Pro- | 299 |
| | CONH2 | |
| 302 | Ac-hLeu-Y-C-V-G-Lys(DOTA)-4COOHPhe-D-V-Nle-C-5FW-F-Pro- | 300 |
| | CONH2 | |
| 303 | Ac-hLeu-Y-C-Tbg-G-K-ThpA-D-V-Y-C-5FW-F-hydroxyVal- | 301 |
| | dLys(DOTA)-COOH | |
| 304 | Ac-hLeu-Y-C-Tbg-G-K-ThpA-D-V-Y-C-5FW-F-Pro-dLys(DOTA)- | 302 |
| | COOH | |
| 305 | Ac-hLeu-Y-C-Tbg-G-Lys(DOTA)-ThpA-D-V-Y-C-5FW-F-Pro-COOH | 303 |
| 306 | DOTA-PEG4-hLeu-Y-C-Tbg-G-K-ThpA-D-V-Y-C-5FW-F-Pro- | 304 |
| | COOH | |
| 307 | Ac-hLeu-Y-C-Tbg-G-K-ThpA-D-V-Y-C-5FW-F-Pro-dLys(PEG4- | 305 |
| | DOTA)-COOH | |
| 308 | Ac-hLeu-Y-C-Ile-G-K-ThpA-D-V-Y-C-5FW-F-hydroxyVal- | 306 |
| | dLys(PEG4-DOTA)-COOH | |
| 309 | Ac-hLeu-Y-C-V-G-Lys(DOTA)-4PyA-D-V-OMeTyr-C-1Nal-F-Pro- | 307 |
| | CONH2 | |
| 310 | Ac-hLeu-Y-C-Tbg-G-K-ThpA-D-V-Y-C-5FW-F-hydroxyVal- | 308 |
| | dLys(PEG4-DOTA)-COOH | |
| 311 | Ac-hLeu-Y-C-Tbg-G-hSer-ThpA-D-V-Y-C-5FW-F-Pro-dLys(DOTA)- | 309 |
| | COOH | |
| 312 | Ac-hLeu-Y-C-Tbg-G-Orn-ThpA-D-V-Y-C-5FW-F-Pro-dLys(DOTA)- | 310 |
| | COOH | |
| 313 | Ac-hLeu-Y-C-V-G-PipAla-4COOHPhe-D-V-Y-C-5FW-F-Pro- | 311 |
| | dLys(DOTA)-COOH | |
| 314 | Ac-hLeu-Y-C-Tbg-G-PipAla-4COOHPhe-D-V-Y-C-1Nal-F-Pro- | 312 |
| | dLys(DOTA)-CONH2 | |
| 315 | Ac-hLeu-Y-C-Tbg-G-PipAla-4COOHPhe-D-V-Y-C-5FW-F-Pro- | 313 |
| | dLys(DOTA)-CONH2 | |
| 316 | Ac-hLeu-Y-C-V-G-PipAla-4COOHPhe-D-V-Y-C-5FW-F-Pro- | 314 |
| | dLys(PEG4-Lys(DOTA)-gGlu-palmitoyl)-CONH2 | |
| 317 | Ac-hLeu-Y-C-V-G-PipAla-4COOHPhe-D-V-Y-C-5FW-F-Pro- | 315 |
| | dLys(PEG24-Lys(DOTA)-gGlu-palmitoyl)-CONH2 | |
| 318 | Ac-hLeu-Y-C-Ile-G-[Pip(CH2CO2H)Ala]-ThpA-D-V-Y-C-5FW-F- | 316 |
| | hydroxyVal-dLys(DOTA)-COOH | |
| 319 | Ac-hLeu-Y-C-Tbg-G-[Pip(CH2CO2H)Ala]-ThpA-D-V-Y-C-5FW-F- | 317 |
| | Pro-dLys(DOTA)-COOH | |
| 320 | Ac-hLeu-Y-C-V-G-[Pip(CH2CO2H)Ala]-4COOHPhe-D-V-Y-C-5FW- | 318 |
| | F-Pro-dLys(DOTA)-CONH2 | |
| 321 | Ac-hLeu-Y-C-Tbg-G-[Pip(CH2CO2H)Ala]-ThpA-D-V-Y-C-5FW-Nle- | 319 |
| | Pro-dLys(DOTA)-COOH | |
| 322 | Ac-hLeu-Y-C-Tbg-G-Lys(DOTA)-ThpA-D-V-Y-C-5FW-F- | 320 |
| | hydroxyVal-COOH | |
| 323 | Ac-hLeu-Y-C-Ile-G-Lys(DOTA)-PipAla-D-V-Y-C-5FW-F- | 321 |
| | hydroxyVal-COOH | |
| 324 | Ac-hLeu-Y-C-Ile-G-Lys(DOTA)-Pip(CH2COOH)Ala-D-V-Y-C-5FW- | 322 |
| | F-hydroxyVal-COOH | |
| 325 | Ac-hLeu-Y-C-V-G-Lys(DOTA)-4COOHPhe-D-V-Y-C-5FW-F- | 323 |
| | hydroxyVal-COOH | |
| 326 | Ac-hLeu-Y-C-Tbg-G-Lys(DOTA)-Pip(CH2COOH)Ala-D-V-Y-C- | 324 |
| | 5FW-F-Thr-COOH | |
| 327 | Ac-hLeu-Y-C-Tbg-G-K-ThpA-D-V-Y-C-5FW-Nle-hydroxyVal- | 325 |
| | dLys(DOTA)-COOH | |
| 328 | Ac-hLeu-Y-C-Tbg-G-Lys(DOTA)-ThpA-D-V-Y-C-5FW-F-Thr-COOH | 326 |
| 329 | Ac-hLeu-Y-S4H-V-G-K-Y-D-V-Y-S4H-5FW-F-Thr-dLys(DOTA)- | 327 |
| | CONH2 | |
| 330 | Ac-hLeu-Y-C(CH2)-Tbg-G-Lys(DOTA)-4PyA-D-V-Y-C-1Nal-F-Pro- | 328 |
| | CONH2 | |
| 331 | Ac-hLeu-Y-C-V-G-K-ThpA-D-V-Y-C-5FW-F-Thr-dLys(DOTA)- | 329 |
| | COOH | |
| 332 | Ac-hLeu-Y-C-Tbg-G-K-ThpA-D-V-Y-C-5FW-F-Thr-dLys(DOTA)- | 330 |
| | COOH | |
| 333 | Ac-hLeu-Y-C-Tbg-G-K-ThpA-D-V-Y-C-5FW-Nle-Thr-dLys(DOTA)- | 331 |
| | COOH | |
| 334 | Ac-hLeu-Y-C-Tbg-G-K-ThpA-D-V-Y-C-5FW-Leu-Thr-dLys(DOTA)- | 332 |
| | COOH | |
| 335 | Ac-hLeu-Y-C-Tbg-G-K-ThpA-D-V-Y-C-5FW-Tbg-Thr-dLys(DOTA)- | 333 |
| | COOH | |
| 336 | Ac-hLeu-Y-C-Tbg-G-Lys(DOTA)-ThpA-D-V-Y-C-5FW-Nle- | 334 |
| | hydroxyVal-CONH2 | |
| 337 | Ac-hLeu-Y-C-Tbg-G-Lys(DOTA)-ThpA-D-V-Y-C-5FW-F- | 335 |
| | hydroxyVal-dLys-CONH2 | |
| 338 | Ac-hLeu-Y-C-Tbg-G-Lys(DOTA)-ThpA-D-V-Y-C-5FW-F- | 336 |
| | hydroxyVal-CONH2 | |
| 339 | Ac-hLeu-Y-C-Tbg-G-[Pip(CH2CO2H)Ala]-ThpA-D-V-Y-C-5FW-F- | 337 |
| | hydroxyVal-dLys(DOTA)-COOH | |
| 340 | Ac-hLeu-Y-C-Tbg-G-PipAla-ThpA-D-V-Y-C-5FW-F-hydroxyVal- | 338 |
| | dLys(DOTA)-COOH | |
| 341 | Ac-hLeu-Y-C-Tbg-G-K(PEG21-COOH)-ThpA-D-V-Y-C-5FW-F-Thr- | 339 |
| | dLys(DOTA)-COOH | |
| 342 | Ac-hLeu-Y-C-Tbg-G-Pip(DOTA)Ala-ThpA-D-V-Y-C-5FW-F- | 340 |
| | hydroxyVal-COOH | |
| 343 | Ac-hLeu-Y-C-Tbg-G-Lys(DOTA)-Pip(CH2COOH)Ala-D-V-Y-C- | 341 |
| | 5FW-F-hydroxyVal-COOH | |
| 344 | Ac-hLeu-Y-C-Tbg-G-K-Pip(DOTA)Ala-D-V-Y-C-5FW-F-hydroxyVal- | 342 |
| | COOH | |
| 345 | Ac-hLeu-Y-C-Tbg-G-K-aMeLeu-D-V-Y-C-5FW-F-Thr-dLys(DOTA)- | 343 |
| | COOH | |
| 347 | Ac-hLeu-Y-C-V-Sar-K-ThpA-D-V-Y-C-5FW-F-hydroxyVal- | 344 |
| | dLys(DOTA)-COOH | |
| 348 | Ac-hLeu-Y-C-Tbg-Sar-K-ThpA-D-V-Y-C-5FW-F-hydroxyVal- | 345 |
| | dLys(DOTA)-COOH | |
| 349 | Ac-hLeu-Y-C-Tbg-G-Pro-ThpA-D-V-Y-C-5FW-F-hydroxyVal- | 346 |
| | dLys(DOTA)-COOH | |
| 350 | Ac-hLeu-Y-C-Tbg-G-Pip-ThpA-D-V-Y-C-5FW-F-hydroxyVal- | 347 |
| | dLys(DOTA)-COOH | |
| 351 | Ac-hLeu-Y-C-Tbg-G-K-ThpA-D-V-Y-(2MercaptorEt)glycine-5FW-F- | 348 |
| | Thr-dLys(DOTA)-COOH | |
| 352 | Ac-hLeu-Y-C-Tbg-G-NMeLys-ThpA-D-V-Y-C-5FW-F-Thr- | 349 |
| | dLys(DOTA)-COOH | |
| 353 | Ac-hLeu-Y-C-Tbg-G-aMeLys-ThpA-D-V-Y-C-5FW-F-Thr- | 350 |
| | dLys(DOTA)-COOH | |
| 354 | Ac-hLeu-Y-C-Tbg-G-K-ThpA-D-aMeVal-Y-C-5FW-F-Thr- | 351 |
| | dLys(DOTA)-COOH | |
| 355 | Ac-hLeu-Y-C-Tbg-G-K-ThpA-D-V-NMeTyr-C-5FW-F-Thr- | 352 |
| | dLys(DOTA)-COOH | |
| 356 | Ac-hLeu-Y-C-Tbg-G-K-ThpA-D-V-aMeTyr-C-5FW-F-Thr- | 353 |
| | dLys(DOTA)-COOH | |
| 357 | Ac-hLeu-Y-C-Tbg-G-K-ThpA-D-V-aMeLeu-C-5FW-F-Thr- | 354 |
| | dLys(DOTA)-COOH | |
| 358 | Ac-hLeu-Y-C-Tbg-G-K-ThpA-D-V-Y-NMeCys-5FW-F-Thr- | 355 |
| | dLys(DOTA)-COOH | |
| 359 | Ac-hLeu-Y-C-Tbg-G-K-ThpA-D-V-Y-C-hF-F-Thr-dLys(DOTA)- | 356 |
| | COOH | |
| 360 | Ac-hLeu-Y-C-Tbg-G-K-ThpA-D-V-Y-C-5FW-tBuAla-Thr- | 357 |
| | dLys(DOTA)-COOH | |
| 361 | Ac-hLeu-Y-NMeCys-V-G-K-ThpA-D-V-Y-C-5FW-F-Thr- | 358 |
| | dLys(DOTA)-COOH | |
| 362 | Ac-hLeu-Y-C-V-Sar-K-ThpA-D-V-Y-C-5FW-F-Thr-dLys(DOTA)- | 359 |
| | COOH | |
| 363 | Ac-hLeu-Y-C-Tbg-G-K(yE-yE-yE-C18OH)-ThpA-D-V-Y-C-5FW-F- | 360 |
| | hydroxyVal-dLys(DOTA)-COOH | |
| 364 | Ac-hLeu-Y-C-Tbg-G-Pip(PEG4-AB)Ala-ThpA-D-V-Y-C-5FW-F- | 361 |
| | hydroxyVal-dLys(DOTA)-COOH | |
| 365 | AB-PEG8-hLeu-Y-C-Tbg-G-K-ThpA-D-V-Y-C-5FW-F-hydroxyVal- | 362 |
| | dLys(DOTA)-COOH | |
| 366 | Ac-hLeu-Y-C-Tbg-G-K-ThpA-D-V-Y-C-5FW-F-hydroxyVal- | 363 |
| | dLys(DOTA)-CONH2 | |
| 367 | Ac-Cha-Y-C-Tbg-G-K-ThpA-D-V-Y-C-5FW-F-hydroxyVal- | 364 |
| | dLys(DOTA)-COOH | |
| 368 | Ac-hCha-Y-C-Tbg-G-K-ThpA-D-V-Y-C-5FW-F-hydroxyVal- | 365 |
| | dLys(DOTA)-COOH | |
| 369 | Ac-hLeu-Y-C-aMeVal-G-K-ThpA-D-V-Y-C-5FW-F-hydroxyVal- | 366 |
| | dLys(DOTA)-COOH | |
| 370 | Ac-hLeu-Y-C-Thr-G-K-ThpA-D-V-Y-C-5FW-F-hydroxyVal- | 367 |
| | dLys(DOTA)-COOH | |
| 371 | Ac-hLeu-Y-C-Tbg-G-Dab-ThpA-D-V-Y-C-5FW-F-hydroxyVal- | 368 |
| | dLys(DOTA)-COOH | |
| 372 | Ac-hLeu-Y-C-Tbg-G-Dap-ThpA-D-V-Y-C-5FW-F-hydroxyVal- | 369 |
| | dLys(DOTA)-COOH | |
| 373 | Ac-hLeu-Y-C-Tbg-G-K-ThpA-D-hSer-Y-C-5FW-F-hydroxyVal- | 370 |
| | dLys(DOTA)-COOH | |
| 374 | Ac-hLeu-Y-C-Tbg-G-dPro-P-D-V-Y-C-5FW-F-hydroxyVal- | 371 |
| | dLys(DOTA)-COOH | |
| 375 | Ac-hLeu-Y-C-Tbg-G-Aze-ThpA-D-V-Y-C-5FW-F-hydroxyVal- | 372 |
| | dLys(DOTA)-COOH | |
| 376 | Ac-hLeu-Y-C-Tbg-G-dPro-ThpA-D-V-Y-C-5FW-F-hydroxyVal- | 373 |
| | dLys(DOTA)-COOH | |
| 377 | Ac-hLeu-Y-C-Tbg-G-K-ThpA-D-Thr-Y-C-5FW-F-hydroxyVal- | 374 |
| | dLys(DOTA)-COOH | |
| 378 | Ac-hLeu-Y-C-Tbg-G-K-ThpA-D-Ser-Y-C-5FW-F-hydroxyVal- | 375 |
| | dLys(DOTA)-COOH | |
| 379 | Ac-hLeu-Y-C-Tbg-G-K-ThpA-D-aMeSer-Y-C-5FW-F-hydroxyVal- | 376 |
| | dLys(DOTA)-COOH | |
| 380 | Ac-hLeu-Y-C-Tbg-G-K-ThpA-D-V-Y-C-5FW-3FPhe-hydroxyVal- | 377 |
| | dLys(DOTA)-COOH | |
| 381 | Ac-hLeu-Y-C-Tbg-G-K-ThpA-D-V-Y-C-5FW-4FPhe-hydroxyVal- | 378 |
| | dLys(DOTA)-COOH | |
| 382 | Ac-hLeu-Y-C-Tbg-G-Pip(PEG4-DOTA)Ala-ThpA-D-V-Y-C-5FW-F- | 379 |
| | hydroxyVal-CONH2 | |
| 383 | Ac-hLeu-Y-C-Tbg-G-K-Pip(PEG4-DOTA)Ala-D-V-Y-C-5FW-F- | 380 |
| | hydroxyVal-CONH2 | |
| 384 | Ac-hLeu-Y-C-Tbg-G-Pip(PEG4-DOTA)Ala-ThpA-D-V-Y-C-5FW-F- | 381 |
| | hydroxyVal-dAla-CONH2 | |
| 385 | Ac-hLeu-Y-C-Tbg-G-K-Pip(PEG4-DOTA)Ala-D-V-Y-C-5FW-F- | 382 |
| | hydroxyVal-dAla-CONH2 | |
| 386 | Ac-hLeu-Y-C-Thr-Sar-K-ThpA-D-V-Y-C-5FW-F-hydroxyVal- | 383 |
| | dLys(DOTA)-COOH | |
| 389 | Ac-hLeu-Y-C-Tbg-G-K-ThpA-D-V-FSY-C-5FW-F-hydroxyVal- | 384 |
| | dLys(DOTA)-COOH | |
| 390 | Ac-hLeu-Y-C-Tbg-G-K-ThpA-FSY-V-Y-C-5FW-F-hydroxyVal- | 385 |
| | dLys(DOTA)-COOH | |
| 391 | DOTA-PEG8-hLeu-Y-C-Tbg-G-K-ThpA-D-V-Y-C-5FW-F- | 386 |
| | hydroxyVal-CONH2 | |
| 392 | DOTA-Glu-AEA-hLeu-Y-C-Tbg-G-K-ThpA-D-V-Y-C-5FW-F- | 387 |
| | hydroxyVal-CONH2 | |
| 393 | DOTA-gE-AEA-hLeu-Y-C-Tbg-G-K-ThpA-D-V-Y-C-5FW-F- | 388 |
| | hydroxyVal-CONH2 | |
| 394 | DOTA-Glu-PEG1-hLeu-Y-C-Tbg-G-K-ThpA-D-V-Y-C-5FW-F- | 389 |
| | hydroxyVal-CONH2 | |
| 395 | DOTA-gE-PEG1-hLeu-Y-C-Tbg-G-K-ThpA-D-V-Y-C-5FW-F- | 390 |
| | hydroxyVal-CONH2 | |
| 396 | Ac-hLeu-Y-C-Tbg-G-K-ThpA-D-V-Y-C-Trp(CH2COOH)-F- | 391 |
| | hydroxyVal-dLys(DOTA)-COOH | |
| 397 | Ac-hTba-Y-C-Tbg-G-K-ThpA-D-V-Y-C-5FW-F-hydroxyVal- | 392 |
| | dLys(DOTA)-COOH | |
| 398 | Ac-hCba-Y-C-Tbg-G-K-ThpA-D-V-Y-C-5FW-F-hydroxyVal- | 393 |
| | dLys(DOTA)-COOH | |
| 399 | Ac-hLeu-Y-C-Tbg-G-K-ThpA-D-V-3OHPhe-C-5FW-F-hydroxyVal- | 394 |
| | dLys(DOTA)-COOH | |
| 400 | Ac-hLeu-Y-C-Tbg-G-K-ThpA-D-V-(4-carbamoyl-Phe)-C-5FW-F- | 395 |
| | hydroxyVal-dLys(DOTA)-COOH | |
| 401 | Ac-hLeu-Y-C-Tbg-G-K-ThpA-D-V-Y-C-5FW-His-hydroxyVal- | 396 |
| | dLys(DOTA)-COOH | |
| 402 | Ac-hLeu-Y-C-Tbg-G-K-ThpA-D-V-Y-C-5FW-4PyA-hydroxyVal- | 397 |
| | dLys(DOTA)-COOH | |
| 403 | Ac-hLeu-Y-C-Tbg-G-K-ThpA-D-V-Y-C-5FW-3PyA-hydroxyVal- | 398 |
| | dLys(DOTA)-COOH | |
| 404 | Ac-hLeu-Y-C-Tbg-G-K-ThpA-D-V-Y-C-5FW-F-hydroxyVal-CONH2 | 399 |
| 405 | Ac-hLeu-Y-C-Tbg-G-K(yE-C16OH)-ThpA-D-V-Y-C-5FW-F- | 400 |
| | hydroxyVal-dLys(DOTA)-COOH | |
| 407 | Ac-hLeu-Y-C-Tbg-G-Pip(PEG4-DOTA)Ala-ThpA-D-V-Y-C-5FW-F- | 401 |
| | hydroxyVal-dSer-CONH2 | |
| 408 | Ac-hLeu-Y-C-Tbg-G-K-Pip(PEG4-DOTA)Ala-D-V-Y-C-5FW-F- | 402 |
| | hydroxyVal-dSer-CONH2 | |
| 409 | Ac-hLeu-Y-C-Tbg-G-Pip(PEG4-DOTA)Ala-ThpA-D-V-Y-C-5FW-F- | 403 |
| | hydroxyVal-dGlu-CONH2 | |
| 410 | Ac-hLeu-Y-C-Tbg-G-K-Pip(PEG4-DOTA)Ala-D-V-Y-C-5FW-F- | 404 |
| | hydroxyVal-dGlu-CONH2 | |
| 419 | Ac-hLeu-Y-C-Tbg-G-Lys(DOTA)-Pip(CH2COOH)Ala-D-V-Y-C- | 405 |
| | 5FW-F-hydroxyVal-CONH2 | |
| 420 | Ac-hLeu-Y-C-Tbg-G-Lys(DOTA)-Pip(CH2COOH)Ala-D-V-Y-C- | 406 |
| | 5FW-F-hydroxyVal-dSer-CONH2 | |
| 421 | Ac-hLeu-Y-C-Tbg-G-Lys(DOTA)-Pip(CH2COOH)Ala-D-V-Y-C- | 407 |
| | 5FW-F-hydroxyVal-dGlu-CONH2 | |
| 422 | Ac-hLeu-Y-C-Tbg-G-Lys(DOTA)-ThpA-D-V-Y-C-5FW-F- | 408 |
| | hydroxyVal-dSer-CONH2 | |
| 423 | Ac-hLeu-Y-C-Tbg-G-Lys(DOTA)-ThpA-D-V-Y-C-5FW-F- | 409 |
| | hydroxyVal-dGlu-CONH2 | |
| 424 | Ac-hLeu-Y-C-Tbg-G-Lys(DOTA)-4OHCha(pk1)-D-V-Y-C-5FW-F- | 410 |
| | hydroxyVal-dGlu-CONH2 | |
| 425 | Ac-hLeu-Y-C-Tbg-G-Lys(DOTA)-4OHCha(pk2)-D-V-Y-C-5FW-F- | 411 |
| | hydroxyVal-dGlu-CONH2 | |
| 427 | Ac-hLeu-Y-C-Tbg-G-PipAla-ThpA-D-V-Aph(Cbm)-C-5FW-F- | 412 |
| | hydroxyVal-dLys(DOTA)-COOH | |
| 428 | Ac-hLeu-Y-C-Tbg-G-PipAla-ThpA-D-V-Y-C-5FW-F-hydroxyVal- | 413 |
| | dLys(Glu-DOTA)-COOH | |
| 429 | Ac-hLeu-Y-C-Tbg-G-PipAla-ThpA-D-V-Y-C-5FW-F-hydroxyVal- | 414 |
| | dLys(dGlu-DOTA)-COOH | |
| 430 | Ac-hLeu-Y-C-Tbg-G-PipAla-ThpA-D-V-Y-C-5FW-3CIPhe- | 415 |
| | hydroxyVal-dLys(DOTA)-COOH | |
| 431 | Ac-hLeu-Y-C-Tbg-G-PipAla-ThpA-D-V-Y-C-5FW-3OHPhe- | 416 |
| | hydroxyVal-dLys(DOTA)-COOH | |
| 432 | Ac-hLeu-Y-C-betaMelle-G-PipAla-ThpA-D-V-Y-C-5FW-F- | 417 |
| | hydroxyVal-dLys(DOTA)-COOH | |
| 433 | Ac-hLeu-Y-C-Tbg-G-PipAla-ThpA-D-V-Y-C-5FW-[(2S, 3S)beta-Me- | 418 |
| | Phe]-hydroxyVal-dLys(DOTA)-COOH | |
| 434 | Ac-hLeu-Y-C-Tbg-G-PipAla-ThpA-D-V-Y-C-5FW-[(2S, 3R)beta- | 419 |
| | Me-Phe]-hydroxyVal-dLys(DOTA)-COOH | |
| 435 | Ac-hLeu-Y-C-Tbg-G-Dab-ThpA-D-V-Y-C-1Nal-F-hydroxyVal- | 420 |
| | dLys(DOTA)-COOH | |
| 436 | Ac-hLeu-Y-C-Tbg-G-PipAla-ThpA-D-V-Y-C-1Nal-F-hydroxyVal- | 421 |
| | dLys(DOTA)-COOH | |
| 437 | MeSO2-hLeu-Y-C-Tbg-G-PipAla-ThpA-D-V-Y-C-5FW-F- | 422 |
| | hydroxyVal-dLys(DOTA)-COOH | |
| 438 | Ac-hLeu-Y-C-Tbg-G-PipAla-ThpA-D-V-Y-C-5FW-F-hydroxyVal- | 423 |
| | dLys(R-DOTAGA)-COOH | |
| 439 | Ac-hLeu-Y-C-Tbg-G-PipAla-ThpA-D-V-Y-C-5FW-F-hydroxyVal- | 424 |
| | dLys(S-DOTAGA)-COOH | |
| 440 | Ac-hLeu-Y-C-Tbg-G-PipAla-ThpA-D-V-Y-C-5FW-F-hydroxyVal- | 425 |
| | dLys(gGlu-DOTA)-COOH | |
| 441 | Ac-hLeu-Y-C-Tbg-G-PipAla-ThpA-D-V-Y-C-1Nal-F-hydroxyVal- | 426 |
| | dLys(gGlu-DOTA)-COOH | |
| 442 | Ac-hCha-Y-C-Tbg-G-PipAla-ThpA-D-V-Y-C-5FW-F-hydroxyVal- | 427 |
| | dLys(DOTA)-COOH | |
| 443 | Ac-hCba-Y-C-Tbg-G-PipAla-ThpA-D-V-Y-C-5FW-F-hydroxyVal- | 428 |
| | dLys(DOTA)-COOH | |
| 444 | Ac-hLeu-Y-C-Tbg-G-PipAla-Pip(PEG4-DOTA)Ala-D-V-Y-C-5FW-F- | 429 |
| | hydroxyVal-dGlu-CONH2 | |
| 445 | Ac-hLeu-Y-C-Tbg-G-PipAla-Pip(PEG4-DOTA)Ala-D-V-Y-C-5FW-F- | 430 |
| | hydroxyVal-CONH2 | |
| 446 | Ac-hLeu-Y-C-Tbg-G-PipAla-Pip(PEG4-DOTA)Ala-D-V-Y-C-5FW-F- | 431 |
| | hydroxyVal-dSer-CONH2 | |
| 447 | Ac-hLeu-Y-C-Tbg-G-PipAla-Pip(DOTA)Ala-D-V-Y-C-5FW-F- | 432 |
| | hydroxyVal-dGlu-CONH2 | |
| 448 | Ac-hLeu-Y-C-Tbg-G-PipAla-Pip(PEG4-DOTA)Ala-D-V-Y-C-5FW-F- | 433 |
| | hydroxyVal-dGlu-COOH | |
| 449 | Ac-hLeu-Y-C-Tbg-G-PipAla-Pip(DOTA)Ala-D-V-Y-C-5FW-F- | 434 |
| | hydroxyVal-dSer-COOH | |
| 450 | Ac-hLeu-Y-C-Tbg-G-PipAla-ThpA-D-V-Y-C-(2S,3S-betaMe-5FW)- | 435 |
| | F-hydroxyVal-dLys(DOTA)-COOH | |
| 451 | C4diacid-hLeu-Y-C-Tbg-G-PipAla-ThpA-D-V-Y-C-5FW-F- | 436 |
| | hydroxyVal-dLys(DOTA)-COOH | |
| 452 | Ac-hLeu-Y-C-Tbg-G-PipAla-ThpA-D-V-Y-C-5FW-F-hydroxyVal- | 437 |
| | dLys(gE-DOTA)-CONH2 | |
| 453 | Ac-hLeu-Y-C-Tbg-G-PipAla-Pip(PEG4-DOTA)Ala-D-V-Y-C-1Nal-F- | 438 |
| | hydroxyVal-dAla-COOH | |
| 454 | Ac-hLeu-Y-C-Tbg-G-PipAla-Pip(PEG4-DOTA)Ala-D-V-Y-C-1Nal-F- | 439 |
| | hydroxyVal-dGlu-CONH2 | |
| 455 | Ac-hLeu-Y-C-Tbg-G-PipAla-Pip(PEG4-DOTA)Ala-D-V-Y-C-5FW-F- | 440 |
| | hydroxyVal-dAla-COOH | |
| 456 | Ac-hLeu-Y-C-Tbg-G-PipAla-ThpA-D-V-Y-C-5FW-2MePhe- | 441 |
| | hydroxyVal-dLys(DOTA)-COOH | |
| 457 | Ac-hLeu-Y-C-Tbg-G-PipAla-ThpA-D-V-Y-C-5ClW-F-hydroxyVal- | 442 |
| | dLys(DOTA)-COOH | |
| 458 | Ac-hLeu-Y-C-Tbg-G-PipAla-Pip(PEG2-DOTA)Ala-D-V-Y-C-5FW-F- | 443 |
| | hydroxyVal-dGlu-CONH2 | |
| 459 | Ac-hLeu-Y-C-Tbg-G-PipAla-Pip(gE-DOTA)Ala-D-V-Y-C-5FW-F- | 444 |
| | hydroxyVal-dGlu-CONH2 | |
| 460 | Ac-hLeu-Y-C-Tbg-G-PipAla-Pip(R-DOTAGA)Ala-D-V-Y-C-5FW-F- | 445 |
| | hydroxyVal-dGlu-CONH2 | |
| 461 | Ac-hLeu-Y-C-Tbg-G-PipAla-ThpA-D-V-Y-C-4FW-F-hydroxyVal- | 446 |
| | dLys(DOTA)-COOH | |
| 462 | Ac-hLeu-Y-C-Tbg-G-PipAla-ThpA-D-V-Y-C-Trp(Me)-F-hydroxyVal- | 447 |
| | dLys(DOTA)-COOH | |
| 463 | Ac-hLeu-Y-C-Tbg-G-PipAla-ThpA-D-V-Y-C-5MeOW-F-hydroxyVal- | 448 |
| | dLys(DOTA)-COOH | |
| 464 | Ac-hLeu-Y-C-Tbg-G-PipAla-ThpA-D-V-Y-C-5FW-F-hydroxyVal- | 449 |
| | dLys(hGlu-DOTA)-COOH | |
| 465 | Ac-hLeu-Y-C-Tbg-G-PipAla-ThpA-D-V-Y-C-5FW-F-hydroxyVal- | 450 |
| | dLys(PEG4-DOTA)-COOH | |
| 467 | Ac-hLeu-Y-C-Tbg-G-PipAla-ThpA-D-V-20HPhe-C-5FW-F- | 451 |
| | hydroxyVal-dLys(DOTA)-COOH | |
| 468 | urea-hLeu-Y-C-Tbg-G-PipAla-ThpA-D-V-Y-C-5FW-F-hydroxyVal- | 452 |
| | dLys(DOTA)-COOH | |
| 469 | Ac-hLeu-Y-C-Tbg-G-PipAla-ThpA-D-V-Y-C-5FW-[(2S, 3R)beta- | 453 |
| | OH-Phe]-hydroxyVal-dLys(DOTA)-COOH | |
| 470 | Ac-hLeu-Y-C-Tbg-G-PipAla-ThpA-D-V-Y-C-5FW-2FPhe- | 454 |
| | hydroxyVal-dLys(DOTA)-COOH | |
| 471 | Ac-hLeu-Y-C-Tbg-G-PipAla-ThpA-D-V-Y-C-5MeW-F-hydroxyVal- | 455 |
| | dLys(DOTA)-COOH | |
| 472 | Ac-hLeu-Y-C-Tbg-G-PipAla-ThpA-D-V-Y-C-4ClW-F-hydroxyVal- | 456 |
| | dLys(DOTA)-COOH | |
| 473 | Ac-hLeu-Y-C-Tbg-G-PipAla-ThpA-D-V-Y-C-4MeW-F-hydroxyVal- | 457 |
| | dLys(DOTA)-COOH | |
| 474 | Ac-hLeu-Y-C-Tbg-G-Pip(PEG2-DOTA)Ala-ThpA-D-V-Y-C-5FW-F- | 458 |
| | hydroxyVal-dGlu-CONH2 | |
| 475 | Ac-hLeu-Y-C-Tbg-G-Pip(gE-DOTA)Ala-ThpA-D-V-Y-C-5FW-F- | 459 |
| | hydroxyVal-dGlu-CONH2 | |
| 476 | Ac-hLeu-Y-C-Tbg-G-Pip(R-DOTAGA)Ala-ThpA-D-V-Y-C-5FW-F- | 460 |
| | hydroxyVal-dGlu-CONH2 | |
| 477 | Pyroglutamate-hLeu-Y-C-Tbg-G-PipAla-ThpA-D-V-Y-C-5FW-F- | 461 |
| | hydroxyVal-dLys(DOTA)-COOH | |
| 478 | Ac-hLeu-FSY-C-Tbg-G-PipAla-ThpA-D-V-Y-C-5FW-F-hydroxyVal- | 462 |
| | dLys(DOTA)-COOH | |
| 479 | Ac-hLeu-Y-C-Tbg-G-PipAla-4OHCha(pk1)-D-V-Y-C-5FW-F- | 463 |
| | hydroxyVal-dLys(DOTA)-COOH | |
| 480 | Ac-hLeu-Y-C-Tbg-G-PipAla-4OHCha(pk2)-D-V-Y-C-5FW-F- | 464 |
| | hydroxyVal-dLys(DOTA)-COOH | |
| 481 | Ac-hLeu-Y-C-Tbg-G-PipAla-ThpA-D-V-Y-C-5FW-F-hydroxyVal- | 465 |
| | dLys(gE-tranexamate-DOTA)-COOH | |
| 482 | Ac-hLeu-Y-C-Tbg-G-PipAla-ThpA-D-V-Y-C-5FW-F-hydroxyVal- | 466 |
| | εLys(Val-Met-AmBz-DOTA)-COOH | |
| 483 | Ac-hLeu-Y-C-Tbg-G-PipAla-Pip(PEG4-DOTA)Ala-D-V-Y-C-5FW-F- | 467 |
| | hydroxyVal-dSer-COOH | |
| 484 | Ac-hLeu-Y-C-Tbg-G-ThpA-Pip(PEG4-DOTA)Ala-D-V-Y-C-5FW-F- | 468 |
| | hydroxyVal-dSer-COOH | |
| 485 | Ac-hCha-Y-C-Tbg-G-Pip(PEG4-DOTA)Ala-ThpA-D-V-Y-C-5FW-F- | 469 |
| | hydroxyVal-dAla-COOH | |
| 486 | Ac-hCha-Y-C-Tbg-G-Pip(gE-DOTA)Ala-ThpA-D-V-Y-C-5FW-F- | 470 |
| | hydroxyVal-dAla-COOH | |
| 487 | Ac-hCha-Y-C-Tbg-G-Pip(gE-gE-DOTA)Ala-ThpA-D-V-Y-C-5FW-F- | 471 |
| | hydroxyVal-dAla-COOH | |
| 488 | Ac-hLeu-Y-C-Tbg-G-Pip(CH2COOH)Ala-Pip(PEG4-DOTA)Ala-D- | 472 |
| | V-Y-C-5FW-F-hydroxyVal-dAla-COOH | |
| 489 | Ac-hLeu-Y-C-Tbg-G-Cit-Pip(PEG4-DOTA)Ala-D-V-Y-C-5FW-F- | 473 |
| | hydroxyVal-dAla-COOH | |
| 490 | Ac-hLeu-Y-C-Tbg-G-Pip(PEG4-DOTA)Ala-ThpA-D-V-Y-C-5FW-F- | 474 |
| | hydroxyVal-dSer-COOH | |
| 491 | Ac-hLeu-Y-C-Tbg-G-PipAla-(N-Me-ThpA)-D-V-Y-C-5FW-F- | 475 |
| | hydroxyVal-dLys(DOTA)-COOH | |
| 492 | Ac-hLeu-Y-C-Tbg-G-PipAla-ThpA-D-V-Y-C-2MeW-F-hydroxyVal- | 476 |
| | dLys(DOTA)-COOH | |
| 493 | Ac-hLeu-Y-C-Tbg-G-PipAla-ThpA-D-V-Y-C-4MeOW-F-hydroxyVal- | 477 |
| | dLys(DOTA)-COOH | |
| 494 | Ac-hLeu-Y-C-Tbg-G-Pip(PEG4-DOTA)Ala-ThpA-D-V-Y-C-5FW- | 478 |
| | [(2S, 3S)beta-Me-Phe]-hydroxyVal-dAla-COOH | |
| 495 | Ac-hLeu-Y-C-Tbg-G-PipAla-Pip(PEG4-DOTA)Ala-D-V-Y-C-5FW- | 479 |
| | [(2S, 3S)beta-Me-Phe]-hydroxyVal-dGlu-CONH2 | |
| 496 | Ac-hLeu-Y-C-Tbg-G-Pip(CH2COOH)Ala-Pip(PEG4-DOTA)Ala-D- | 480 |
| | V-Y-C-5FW-[(2S, 3S) beta-Me-Phe]-hydroxyVal-dGlu-CONH2 | |
| 497 | Ac-hLeu-Y-C-Tbg-G-Pip(CH2COOH)Ala-ThpA-D-V-Y-C-5FW-[(2S, | 481 |
| | 3S)beta-Me-Phe]-hydroxyVal-dLys(DOTA)-COOH | |
| 498 | Ac-hLeu-Y-C-Tbg-G-PipAla-ThpA-D-V-Y-C-5FW-[(2S, 3S)beta-Me- | 482 |
| | Phe]-hydroxyVal-dLys(Glu-DOTA)-COOH | |
| 499 | Ac-hLeu-Y-C-Tbg-G-Pip(CH2COOH)Ala-ThpA-D-V-Aph(Cbm)-C- | 483 |
| | 5FW-[(2S, 3S)beta-Me-Phe]-hydroxyVal-dLys(DOTA)-COOH | |
| 500 | Ac-hLeu-Y-C-Tbg-G-PipAla-ThpA-D-V-Aph(Cbm)-C-5FW-[(2S, | 484 |
| | 3S)beta-Me-Phe]-hydroxyVal-dLys(PEG4-DOTA)-COOH | |
| 501 | Ac-hLeu-Y-C-Tbg-G-PipAla-ThpA-D-V-Y-C-5FW-[(2S, 3S)beta-Me- | 485 |
| | Phe]-hydroxyVal-dLys(dGlu-DOTA)-COOH | |
| 502 | Ac-hLeu-Y-C-Tbg-G-PipAla-ThpA-D-V-Y-C-5FW-[(2S, 3S)beta-Me- | 486 |
| | Phe]-hydroxyVal-dLys(DOTA)-CONH2 | |
| 503 | Ac-hLeu-Y-C-Tbg-G-PipAla-ThpA-D-V-Aph(Cbm)-C-5FW-[(2S, | 487 |
| | 3S)beta-Me-Phe]-hydroxyVal-dLys(Glu-DOTA)-COOH | |
| 504 | Pyroglutamate-hLeu-Y-C-Tbg-G-PipAla-ThpA-D-V-Aph(Cbm)-C- | 488 |
| | 5FW-[(2S, 3S) beta-Me-Phe]-hydroxyVal-dLys(DOTA)-COOH | |
| 505 | Pyroglutamate-hLeu-Y-C-Tbg-G-PipAla-ThpA-D-V-Aph(Cbm)-C- | 489 |
| | 5FW-[(2S, 3S)beta-Me-Phe]-hydroxyVal-dLys(Glu-DOTA)-COOH | |
| 506 | Ac-hLeu-Y-C-Tbg-G-PipAla-ThpA-D-V-Aph(Cbm)-C-5FW-[(2S, | 490 |
| | 3S)beta-Me-Phe]-hydroxyVal-dLys(DOTA)-COOH | |
| 507 | Pyroglutamate-hLeu-Y-C-Tbg-G-PipAla-ThpA-D-V-Y-C-5FW-[(2S, | 491 |
| | 3S)beta-Me-Phe]-hydroxyVal-dLys(DOTA)-COOH | |
| 508 | Ac-hLeu-Y-C-Tbg-G-PipAla-ThpA-D-V-Aph(Cbm)-C-5FW-[(2S, | 492 |
| | 3S)beta-Me-Phe]-hydroxyVal-dLys(DOTA)-dGlu-COOH | |
| 509 | Ac-hLeu-Y-C-Tbg-G-PipAla-ThpA-D-V-Y-C-5FW-[(2S, 3S)beta-Me- | 493 |
| | Phe]-hydroxyVal-Lys(DOTA)-COOH | |
| 510 | Ac-hLeu-Y-C-Tbg-G-PipAla-Pip(PEG4-DOTA)Ala-D-V-Y-C-5FW- | 494 |
| | [(2S, 3S)beta-Me-Phe]-hydroxyVal-Gly-COOH | |
| 511 | Ac-hLeu-Y-C-Tbg-G-PipAla-Pip(PEG4-DOTA)Ala-D-V-Y-C-5FW- | 495 |
| | [(2S, 3S)beta-Me-Phe]-hydroxyVal-betaAla-COOH | |
| 512 | Ac-hLeu-Y-C-Tbg-G-Lys(Ac)-Pip(PEG4-DOTA)Ala-D-V-Y-C-5FW- | 496 |
| | [(2S, 3S)beta-Me-Phe]-hydroxyVal-dAla-COOH | |
| 513 | Ac-hLeu-Y-C-Tbg-G-Pip(Ac)Ala-Pip(PEG4-DOTA)Ala-D-V-Y-C- | 497 |
| | 5FW-[(2S, 3S)beta-Me-Phe]-hydroxyVal-dAla-COOH | |
| 514 | Ac-hLeu-Y-C-Tbg-G-Pip(CONH2)Ala-Pip(PEG4-DOTA)Ala-D-V-Y- | 498 |
| | C-5FW-[(2S, 3S)beta-Me-Phe]-hydroxyVal-dAla-COOH | |
| 515 | Ac-hLeu-Y-C-Tbg-G-PipAla-Pip(PEG4-Ac)Ala-D-V-Y-C-5FW-[(2S, | 499 |
| | 3S)beta-Me-Phe]-hydroxyVal-dLys(DOTA)-COOH | |
| 516 | Ac-hLeu-Y-C-Tbg-G-PipAla-ThpA-D-V-Y-C-1Nal-[(2S, 3S)beta-Me- | 500 |
| | Phe]-hydroxyVal-dLys(DOTA)-COOH | |
| 517 | Ac-hLeu-Y-C-Tbg-G-PipAla-ThpA-D-V-Y-C-5FW-[(2S, 3S)beta-Me- | 501 |
| | Phe]-hydroxyVal-&Lys(Val-Met-AmBz-DOTA)-COOH | |
| 518 | Ac-hLeu-Y-C-Tbg-G-PipAla-Glu[&Lys(OH)-Val-Met-AmBz-DOTA]- | 502 |
| | D-V-Y-C-5FW-[(2S, 3S)beta-Me-Phe]-hydroxyVal-dSer-COOH | |
| 519 | Ac-hLeu-Y-C-Tbg-G-Glu[&Lys(OH)-Val-Met-AmBz-DOTA]-ThpA-D- | 503 |
| | V-Y-C-5FW-[(2S, 3S)beta-Me-Phe]-hydroxyVal-dSer-COOH | |
| 520 | Ac-hLeu-Y-C-Tbg-G-Pip(Me)Ala-Pip(PEG4-DOTA)Ala-D-V-Y-C- | 504 |
| | 5FW-[(2S, 3S) beta-Me-Phe]-hydroxyVal-dAla-COOH | |
| 521 | Ac-hLeu-Y-C-Tbg-G-Lys(Me)2-Pip(PEG4-DOTA)Ala-D-V-Y-C- | 505 |
| | 5FW-[(2S, 3S)beta-Me-Phe]-hydroxyVal-dAla-COOH | |
| 522 | Ac-hLeu-Y-C-Tbg-G-Lys(Me)3-Pip(PEG4-DOTA)Ala-D-V-Y-C- | 506 |
| | 5FW-[(2S, 3S)beta-Me-Phe]-hydroxyVal-dAla-COOH | |
| 523 | Ac-hLeu-Y-C-Tbg-G-PipAla-Lys(PEG4-DPTA)-D-V-Y-C-5FW-[(2S, | 507 |
| | 3S)beta-Me-Phe]-hydroxyVal-dAla-COOH | |
| 524 | Ac-hLeu-Y-C-Tbg-G-PipAla-ThpA-CysAcid-V-Y-C-5FW-[(2S, | 508 |
| | 3S)beta-Me-Phe]-hydroxyVal-dLys(DOTA)-COOH | |
| 525 | Ac-hLeu-Y-C-Tbg-G-Pip(CH2COOH)Ala-ThpA-D-V-Y-C-5FW-[(2S, | 509 |
| | 3S)beta-Me-Phe]-hydroxyVal-dLys(hGlu-DOTA)-COOH | |
| 526 | Ac-hLeu-Y-C-Tbg-G-Pip(CH2COOH)Ala-Pip(PEG4-DOTA)Ala-D- | 510 |
| | V-Y-C-5FW-[(2S, 3S) beta-Me-Phe]-hydroxyVal-dAla-COOH | |
| 527 | Ac-hLeu-Y-C-betaMelle-G-PipAla-ThpA-D-V-Y-C-5FW-[(2S, | 511 |
| | 3S)beta-Me-Phe]-hydroxyVal-dLys(DOTA)-COOH | |
| 528 | Ac-hLeu-Y-C-Tbg-G-PipAla-ThpA-D-V-Y-C-5FW-[(2S, 3S)beta-Me- | 512 |
| | Phe]-hydroxyVal-dLys(hGlu-DOTA)-COOH | |
| 529 | Ac-hLeu-Y-C-Tbg-G-PipAla-ThpA-D-V-Y-C-5FW-[(2S, 3S)beta-Me- | 513 |
| | Phe]-Pro-dLys(hGlu-DOTA)-COOH | |
| 530 | Ac-hLeu-Y-C-Tbg-G-PipAla-ThpA-D-V-Y-C-5FW-[(2S, 3S)beta-Me- | 514 |
| | Phe]-hydroxyVal-dLys(hGlu-hGlu-DOTA)-COOH | |
| 531 | Ac-hLeu-Y-C-Tbg-G-PipAla-(N-Me-ThpA)-D-V-Y-C-5FW-[(2S, | 515 |
| | 3S)beta-Me-Phe]-hydroxyVal-dLys(DOTA)-COOH | |
| 532 | Ac-hLeu-Y-C-Tbg-G-PipAla-Pip(CH2COOH)Ala-D-V-Y-C-5FW- | 516 |
| | [(2S, 3S)beta-Me-Phe]-hydroxyVal-dLys(DOTA)-COOH | |
| 533 | Ac-hLeu-Y-C-Tbg-G-Pip(CH2COOH)Ala-Pip(CH2COOH)Ala-D-V- | 517 |
| | Y-C-5FW-[(2S, 3S)beta-Me-Phe]-hydroxyVal-dLys(DOTA)-COOH | |
| 534 | Ac-hLeu-Y-C-Tbg-G-PipAla-Pip(C4diacid)Ala-D-V-Y-C-5FW-[(2S, | 518 |
| | 3S)beta-Me-Phe]-hydroxyVal-dLys(DOTA)-COOH | |
| 535 | Ac-hLeu-Y-C-Tbg-G-PipAla-Pip(PEG4-C4diacid) Ala-D-V-Y-C- | 519 |
| | 5FW-[(2S, 3S)beta-Me-Phe]-hydroxyVal-dLys(DOTA)-COOH | |
| 536 | Ac-hLeu-Y-C-Tbg-G-PipAla-Pip(PEG4-DOTA)Ala-D-V-Y-C-5FW- | 520 |
| | [(2S, 3S)beta-Me-Phe]-hydroxyVal-dAla-COOH | |
| 537 | Ac-hLeu-Y-C-Tbg-G-PipAla-Pip(PEG4-DOTA)Ala-D-V-Y-C-5FW- | 521 |
| | [(2S, 3S)beta-Me-Phe]-hydroxyVal-dLys(DOTA)-COOH | |
| 538 | Ac-hLeu-Y-C-Tbg-G-Pip(PEG4-DOTA)Ala-ThpA-D-V-Y-C-5FW- | 522 |
| | [(2S, 3S)beta-Me-Phe]-hydroxyVal-dLys(DOTA)-COOH | |
| 539 | Ac-hLeu-Y-C-Tbg-G-PipAla-ThpA-D-V-Y-C-5FW-[(2S, 3S)beta-Me- | 523 |
| | Phe]-Pro-dLys(DOTA)-COOH | |
| 540 | Ac-hLeu-Y-C-Tbg-G-ThpA-Pip(PEG4-DOTA)Ala-D-V-Y-C-5FW- | 524 |
| | [(2S, 3S)beta-Me-Phe]-hydroxyVal-dAla-COOH | |
| 541 | Ac-hLeu-Y-C-Tbg-G-Pip(PEG4-DOTA)Ala-ThpA-D-V-Aph(Cbm)-C- | 525 |
| | 5FW-[(2S, 3S)beta-Me-Phe]-hydroxyVal-dAla-COOH | |
| 542 | Ac-hLeu-Y-C-Tbg-G-Pip(CH2COOH)Ala-Pip(PEG4-DOTA)Ala-D- | 526 |
| | V-Aph(Cbm)-C-5FW-[(2S, 3S)beta-Me-Phe]-hydroxyVal-dAla- | |
| | COOH | |
| 543 | Ac-hLeu-Y-C-Tbg-G-PipAla-ThpA-D-V-Y-C-5FW-[(2S, 3S)beta-Me- | 527 |
| | Phe]-hydroxyVal-dLys(hGlu-hGlu-hGlu-DOTA)-COOH | |
| 544 | Ac-hLeu-Y-C-Tbg-G-Pip(C4diacid)Ala-ThpA-D-V-Y-C-5FW-[(2S, | 528 |
| | 3S)beta-Me-Phe]-hydroxyVal-dLys(hGlu-DOTA)-COOH | |
| 545 | Ac-hLeu-Y-C-Tbg-G-Pip(CH2COOH)Ala-ThpA-D-V-Y-C-5FW-[(2S, | 529 |
| | 3S)beta-Me-Phe]-hydroxyVal-dLys(hGlu-DOTA)-dGlu-COOH | |
| 546 | Ac-hLeu-Y-C-Tbg-G-Pip(CH2COOH)Ala-ThpA-D-V-Y-C-5FW-[(2S, | 530 |
| | 3S)beta-Me-Phe]-hydroxyVal-dLys(hGlu-transhexamate-DOTA)- | |
| | COOH | |
| 547 | Ac-hLeu-Y-C-Tbg-G-Pip(CH2COOH)Ala-ThpA-D-V-Aph(Cbm)-C- | 531 |
| | 5FW-[(2S, 3S)beta-Me-Phe]-hydroxyVal-dLys(hGlu-DOTA)-COOH | |
| 548 | Ac-hLeu-Y-C-Tbg-G-Pip(CH2COOH)Ala-Lys(DOTA)-D-V-Y-C- | 532 |
| | 5FW-[(2S, 3S)beta-Me-Phe]-hydroxyVal-dAla-COOH | |
| 549 | Ac-hLeu-Y-C-Tbg-G-Pip(CH2COOH)Ala-Pip(DOTA)Ala-D-V-Y-C- | 533 |
| | 5FW-[(2S, 3S)beta-Me-Phe]-hydroxyVal-dAla-COOH | |
| 550 | Ac-hLeu-Y-C-Tbg-G-Pip(CH2COOH)Ala-Pip(Glu-DOTA)Ala-D-V- | 534 |
| | Y-C-5FW-[(2S, 3S)beta-Me-Phe]-hydroxyVal-dAla-COOH | |
| 551 | Ac-hLeu-Y-C-Tbg-G-Pip(CH2COOH)Ala-ThpA-D-V-Aph(Cbm)-C- | 535 |
| | 5FW-[(2S, 3S)beta-Me-Phe]-hydroxyVal-dLys(DOTA)-dGlu-COOH | |
| 552 | Ac-Nle-Y-C-V-G-K-Y-Dab-V-Y-C-W-F-Thr-dLys(DOTA)-CONH2 | 536 |

or a pharmaceutically acceptable salt thereof.

Note: C, dCys, NMeCys, and/or Pen in the above sequences in Table 2 and below indicates the two Cysteine residues form a cyclic bond through a disulfide bridge.

Note: C(CH₂) in the above sequences in Table 2 and below indicates the two Cysteine residues form a cyclic bond through a thioacetal bridge (S—CH₂—S).

Note: Pra and DAPN3 in the above sequences in Table 2 and below indicates the two residues involved in cyclic bond formation with a triazole bridge (e.g.,

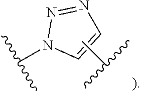

Note: S4H in the above sequences in Table 2 and below indicates the two residues involved in cyclic bond formation with an alkylene bridge (e.g.,

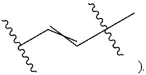 [SMILES: *C=C*]

In an embodiment, the cyclic peptide of Formula I is selected from Examples 100, 136, 142-145, 169, 182, 183, 191, 201-203, 228-230, 232, 233, 249-258, 260-269, 273, 279, 289, 290, 292, 295, 296, 301, 302, 305, 309, 322-326, 328, 330, 336-338, 342-344, 382-385, 407-410, 420-425, 444-449, 453-455, 458-460, 474-476, 483-490, 494-496, 510-514, 518-523, 526, 536-538, 540-542, and 548-550 of Table 1.

In another embodiment, the cyclic peptide of Formula VI is selected from Examples 1-99, 101-135, 137-141, 146-168, 170-181, 184-190, 192-200, 204-227, 231, 234-248, 259, 271, 272, 274-278, 280-288, 291, 293, 294, 297-300, 303, 304, 306-308, 310-321, 327, 329, 331-335, 339-341, 345, 347-381, 386, 389, 405, 427-443, 450-452, 456, 457, 461-465, 467-473, 477-482, 491-493, 497-509, 515-517, 524, 525, 527-535, 539, 543-547, 551, and 552 of Table 1.

In yet another embodiment, the cyclic peptide of Formula I is selected from a cyclic peptide in Table B, or a pharmaceutically acceptable salt and/or solvate thereof.

In yet another embodiment, the cyclic peptide of Formula I is selected from a peptide in Table A.

TABLE A

| | Cpd. |
|---|---|
| Structure | No |
| 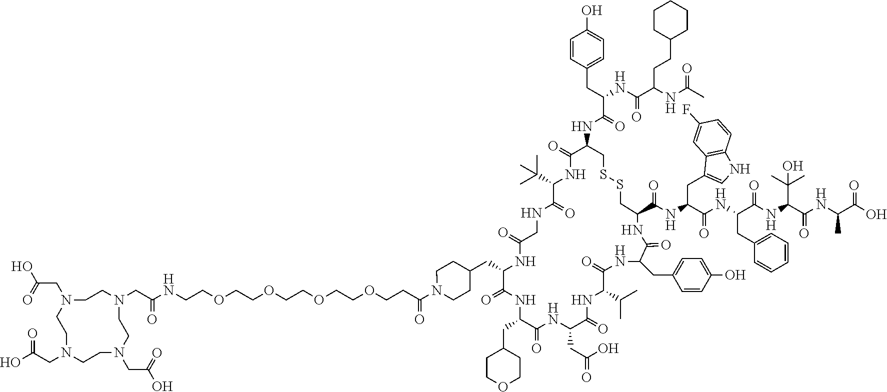 [SMILES: CC(=O)NC(CCC1CCCCC1)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@@H](Cc2ccccc2)C(=O)N[C@H](C(=O)N[C@H](C)C(=O)O)C(C)(C)O)NC(=O)C(Cc2ccc(O)cc2)NC(=O)[C@H](C(C)C)NC(=O)[C@H](CC(=O)O)NC(=O)[C@H](CC2CCOCC2)NC(=O)[C@H](CC2CCN(C(=O)CCOCCOCCOCCOCCNC(=O)CN3CCN(CC(=O)O)CCN(CC(=O)O)CCN(CC(=O)O)CC3)CC2)NC(=O)CNC(=O)[C@H](C(C)(C)C)NC1=O] | 485 |
| 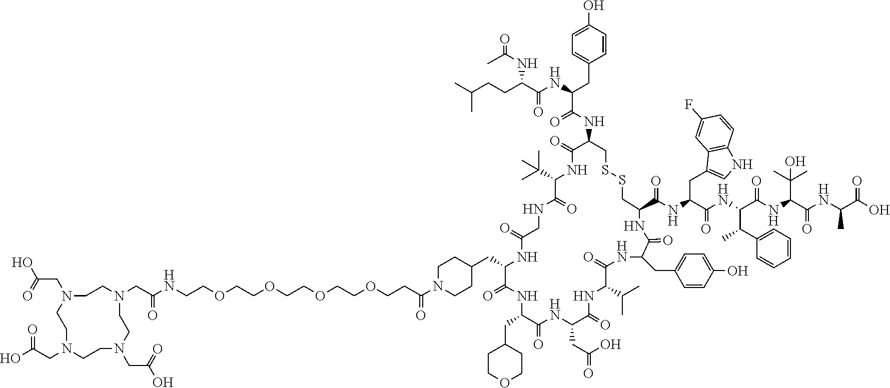 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@H](C(=O)N[C@H](C(=O)N[C@H](C)C(=O)O)C(C)(C)O)[C@@H](C)c2ccccc2)NC(=O)C(Cc2ccc(O)cc2)NC(=O)[C@H](C(C)C)NC(=O)[C@H](CC(=O)O)NC(=O)[C@H](CC2CCOCC2)NC(=O)[C@H](CC2CCN(C(=O)CCOCCOCCOCCOCCNC(=O)CN3CCN(CC(=O)O)CCN(CC(=O)O)CCN(CC(=O)O)CC3)CC2)NC(=O)CNC(=O)[C@H](C(C)(C)C)NC1=O] | 494 |
| 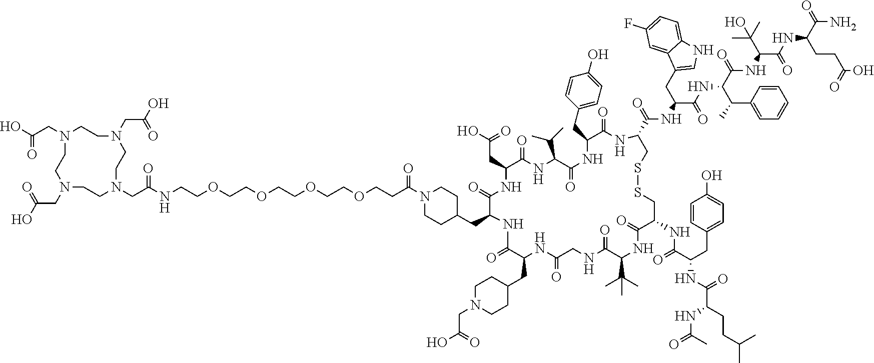 [SMILES: CC(=O)N[C@@H](CCC(C)C)C(=O)N[C@@H](Cc1ccc(O)cc1)C(=O)N[C@H]1CSSC[C@@H](C(=O)N[C@@H](Cc2c[nH]c3ccc(F)cc23)C(=O)N[C@H](C(=O)N[C@H](C(=O)N[C@H](CCC(=O)O)C(N)=O)C(C)(C)O)[C@@H](C)c2ccccc2)NC(=O)[C@H](Cc2ccc(O)cc2)NC(=O)[C@H](C(C)C)NC(=O)[C@H](CC(=O)O)NC(=O)[C@H](CC2CCN(C(=O)CCOCCOCCOCCOCCNC(=O)CN3CCN(CC(=O)O)CCN(CC(=O)O)CCN(CC(=O)O)CC3)CC2)NC(=O)[C@H](CC2CCN(CC(=O)O)CC2)NC(=O)CNC(=O)[C@H](C(C)(C)C)NC1=O] | 496 |
| 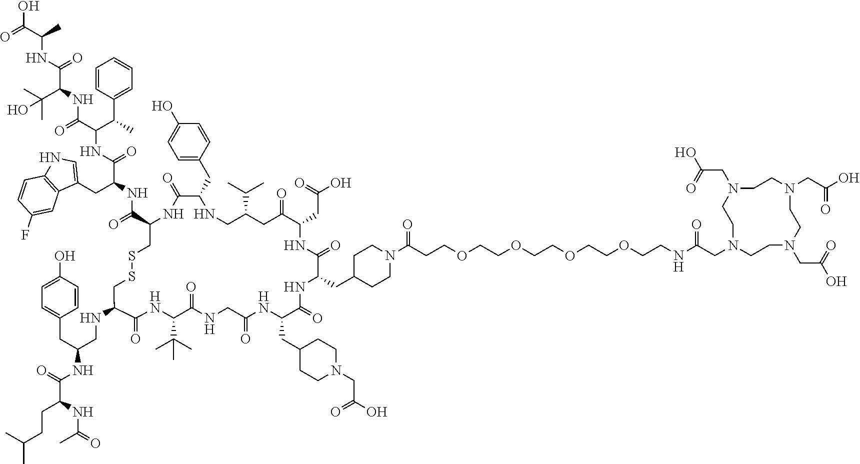 [SMILES: CC(=O)N[C@@H](CCC(C)C)C(=O)N[C@H](CN[C@H]1CSSC[C@@H](C(=O)N[C@@H](Cc2c[nH]c3ccc(F)cc23)C(=O)NC(C(=O)N[C@H](C(=O)N[C@H](C)C(=O)O)C(C)(C)O)[C@@H](C)c2ccccc2)NC(=O)[C@H](Cc2ccc(O)cc2)NC[C@H](C(C)C)CC(=O)[C@H](CC(=O)O)NC(=O)[C@H](CC2CCN(C(=O)CCOCCOCCOCCOCCNC(=O)CN3CCN(CC(=O)O)CCN(CC(=O)O)CCN(CC(=O)O)CC3)CC2)NC(=O)[C@H](CC2CCN(CC(=O)O)CC2)NC(=O)CNC(=O)[C@H](C(C)(C)C)NC1=O)Cc1ccc(O)cc1] | 526 |

In still another embodiment, the cyclic peptide of Formula I is selected from a peptide in Table B.

TABLE B

| | Cpd |
|---|---|
| Structure | No. |
| 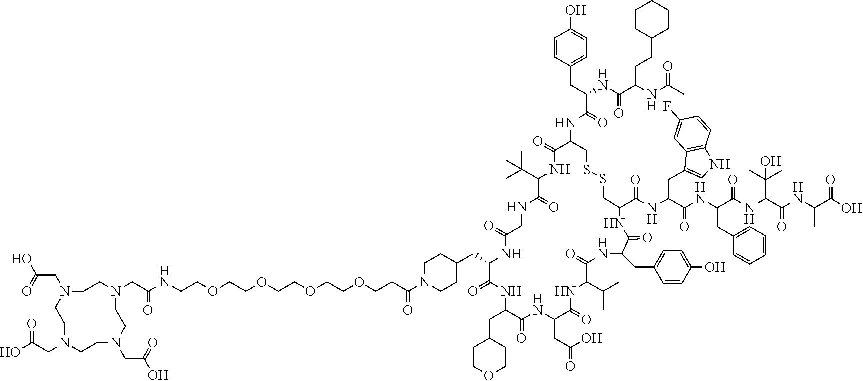 [SMILES: CC(=O)NC(CCC1CCCCC1)C(=O)N[C@@H](Cc1ccc(O)cc1)C(=O)NC1CSSCC(C(=O)NC(Cc2c[nH]c3ccc(F)cc23)C(=O)NC(Cc2ccccc2)C(=O)NC(C(=O)NC(C)C(=O)O)C(C)(C)O)NC(=O)C(Cc2ccc(O)cc2)NC(=O)C(C(C)C)NC(=O)C(CC(=O)O)NC(=O)C(CC2CCOCC2)NC(=O)[C@H](CC2CCN(C(=O)CCOCCOCCOCCOCCNC(=O)CN3CCN(CC(=O)O)CCN(CC(=O)O)CCN(CC(=O)O)CC3)CC2)NC(=O)CNC(=O)C(C(C)(C)C)NC1=O] | 485a |
| 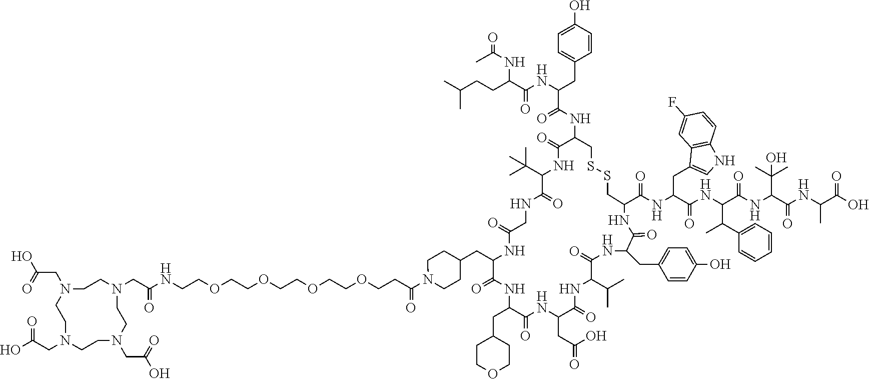 [SMILES: CC(=O)NC(CCC(C)C)C(=O)NC(Cc1ccc(O)cc1)C(=O)NC1CSSCC(C(=O)NC(Cc2c[nH]c3ccc(F)cc23)C(=O)NC(C(=O)NC(C(=O)NC(C)C(=O)O)C(C)(C)O)C(C)c2ccccc2)NC(=O)C(Cc2ccc(O)cc2)NC(=O)C(C(C)C)NC(=O)C(CC(=O)O)NC(=O)C(CC2CCOCC2)NC(=O)C(CC2CCN(C(=O)CCOCCOCCOCCOCCNC(=O)CN3CCN(CC(=O)O)CCN(CC(=O)O)CCN(CC(=O)O)CC3)CC2)NC(=O)CNC(=O)C(C(C)(C)C)NC1=O] | 494a |
| 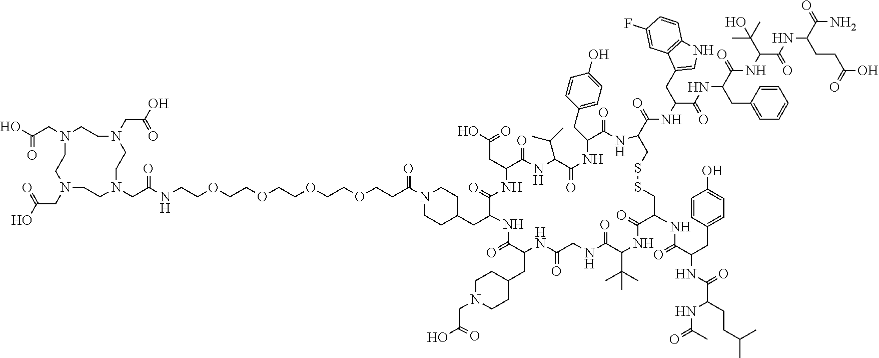 [SMILES: CC(=O)NC(CCC(C)C)C(=O)NC(Cc1ccc(O)cc1)C(=O)NC1CSSCC(C(=O)NC(Cc2c[nH]c3ccc(F)cc23)C(=O)NC(Cc2ccccc2)C(=O)NC(C(=O)NC(CCC(=O)O)C(N)=O)C(C)(C)O)NC(=O)C(Cc2ccc(O)cc2)NC(=O)C(C(C)C)NC(=O)C(CC(=O)O)NC(=O)C(CC2CCN(C(=O)CCOCCOCCOCCOCCNC(=O)CN3CCN(CC(=O)O)CCN(CC(=O)O)CCN(CC(=O)O)CC3)CC2)NC(=O)C(CC2CCN(CC(=O)O)CC2)NC(=O)CNC(=O)C(C(C)(C)C)NC1=O] | 496a |
| 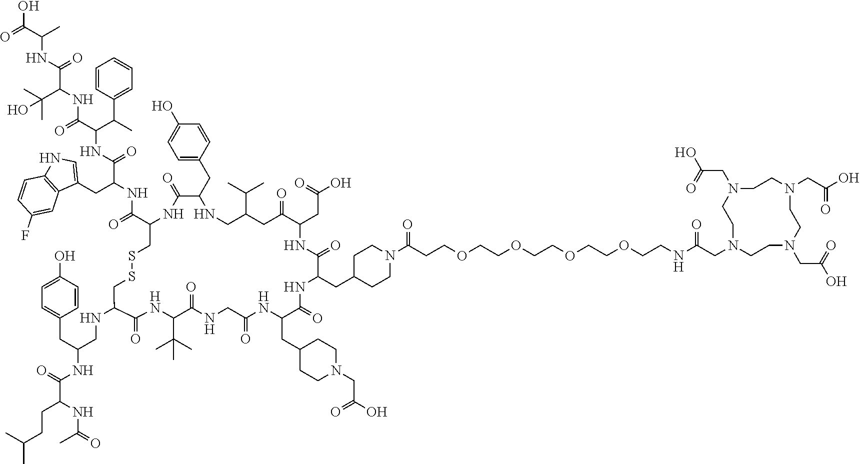 [SMILES: CC(=O)NC(CCC(C)C)C(=O)NC(CNC1CSSCC(C(=O)NC(Cc2c[nH]c3ccc(F)cc23)C(=O)NC(C(=O)NC(C(=O)NC(C)C(=O)O)C(C)(C)O)C(C)c2ccccc2)NC(=O)C(Cc2ccc(O)cc2)NCC(C(C)C)CC(=O)C(CC(=O)O)NC(=O)C(CC2CCN(C(=O)CCOCCOCCOCCOCCNC(=O)CN3CCN(CC(=O)O)CCN(CC(=O)O)CCN(CC(=O)O)CC3)CC2)NC(=O)C(CC2CCN(CC(=O)O)CC2)NC(=O)CNC(=O)C(C(C)(C)C)NC1=O)Cc1ccc(O)cc1] | 526a |

In another aspect, provided herein is a pharmaceutical composition comprising a peptide described herein, or a pharmaceutically acceptable salt thereof, and a pharmaceutically acceptable carrier.

The compounds disclosed herein may exist as tautomers and optical isomers (e.g., enantiomers, diastereomers, diastereomeric mixtures, racemic mixtures, and the like). The absolute stereochemistry is specified according to the Cahn-Ingold-Prelog R-S system. Chiral centers, of which the absolute configurations are known, are labelled by prefixes R and S, assigned by the standard sequence-rule procedure, and preceded when necessary by the appropriate locants (Pure & Appl. Chem. 45, 1976, 11-30). Certain examples contain chemical structures that are depicted or labelled as an (R*) or (S*). When (R*) or (S*) is used in the name of a compound or in the chemical representation of the compound, it is intended to convey that the compound is a pure single isomer at that stereocenter; however, absolute configuration of that stereocenter has not been established. Thus, a compound designated as (R*) refers to a compound that is a pure single isomer at that stereocenter with an absolute configuration of either (R) or(S), and a compound designated as (S*) refers to a compound that is a pure single isomer at that stereocenter with an absolute configuration of either (R) or(S).

Compounds provided herein can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can used in various studies such as NMR spectroscopy, metabolism experiments, and/or assays.

In the compounds provided herein, any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Also, unless otherwise stated, when a position is designated specifically as “D” or “deuterium”, the position is understood to have deuterium at an abundance that is at least 3000 times greater than the natural abundance of deuterium, which is 0.015% (i.e., at least 45% incorporation of deuterium).

In embodiments, the compounds provided herein have an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Examples of bridging moieties/peptide staples for use with compounds of the present disclosure include, but are not limited to: amide-based (e.g., lactam) bridges; aromatic-ring-based bridges; hydrocarbon chains; alkene-based hydrocarbon bridges (e.g., using Fmoc-S-2-(2′-pentenyl)alanine); triazole-based Click bridges, such as copper(I)-catalyzed Huisgen 1,3-dipolar cycloaddition reactions between side chain azido and alkynyl moieties (e.g., Fmoc-L-Nle(εN3) and Fmoc-D-Pra) (see S. Kawamoto, et al., J. Med. Chem. 2012, 55(3), 1137-1146); dialkynyl staples (e.g., 1,4-diethynylbenzene, diethynylpentane, diethynylamines) for stapling linear diazido-peptides; sulfide-bonded disulfide, thioether and bis-thioether bridges; perfluorobenzene bridges; or combinations thereof.

In some embodiments, bridging moieties comprise an amide bond between an amine functionality and a carboxylate functionality, each present in an amino acid, unnatural amino acid or non-amino acid residue side chain. In some embodiments, the amine or carboxylate functionalities are part of a non-amino acid residue or unnatural amino acid residue. In some embodiments, the bridging moiety comprises an amide bond produced by the reaction of the side chains of the following pairs of amino acids: lysine and glutamate; lysine and aspartate; ornithine and glutamate; ornithine and aspartate; homolysine and glutamic acid; homolysine and aspartic acid; and other combinations of amino acids, unnatural amino acids or non-amino acid residues comprising a primary amine and a carboxylic acid. In some embodiments, bridging moieties are formed through cyclization reactions using olefin metathesis.

In some embodiments, the bridging moiety comprises a disulfide bond formed between two thiol containing residues. In some embodiments, the bridging moiety comprises one or more thioether bonds. Such thioether bonds may include those found in cyclo-thioalkyl compounds. These bonds can be formed during a chemical cyclization reaction between chloro acetic acid N-terminal modified groups and cysteine residues. In some embodiments, bridging moieties comprise one or more triazole ring.

In some embodiments, bridging moieties comprise one or more hydrocarbon chains (linear or branched), and/or hydrocarbon rings (cyclic, heterocyclic, aromatic, heteroaromatic). In some embodiments, hydrocarbon bridging moieties may be introduced by reaction with reagents containing multiple reactive halides, including, but not limited to poly(bromomethyl)benzenes, poly(bromomethyl)pyridines, poly(bromomethyl)alkyl benzenes and/or (E)-1,4-dibromobut-2-ene. Examples of Poly(bromomethyl)benzene molecules of the present disclosure can include 1,2-bis(bromomethyl)benzene; 1,3-bis(bromomethyl)benzene; and 1,4-bis(bromomethyl)benzene.

In some embodiments, the thiol group of a cysteine residue is cross-linked with another cysteine residue to form a disulfide bond. In some embodiments, thiol groups of cysteine residues react with bromomethyl groups of poly(bromomethyl)benzene molecules to form stable linkages (see, e.g., Timmerman et al., ChemBioChem (2005) 6:821-824, the contents of which are incorporated herein by reference in their entirety).

In some embodiments, Bis-, tris- and tetrakis(bromomethyl)benzene molecules can be used to generate bridging moieties to produce peptides with one, two or three loops, respectively. Bromomethyl groups of a poly(bromomethyl)benzene molecule may be arranged on the benzene ring on adjacent ring carbons (ortho- or o-), with a ring carbon separating the two groups (meta- or m-) or on opposite ring carbons (para- or p-). In some embodiments, m-bis(bromomethyl)benzene (i.e., m-dibromoxylene), o-bis(bromomethyl)benzene (i.e., o-dibromoxylene) and/or p-bis(bromomethyl)benzene (i.e., p-dibromoxylene) are used to form cyclic peptides. In some embodiments, thiol groups of cysteine residues react with other reagents comprising one or more bromo functional groups to form stable linkages. Such reagents may include, but are not limited to poly(bromomethyl)pyridines (e.g., 2,6-bis(bromomethyl)pyridine), poly(bromomethyl)alkyl benzenes (e.g., 1,2-bis(bromomethyl)-4-alkylbenzene) and/or (E)-1,4-dibromobut-2-ene.

In some embodiments, a side chain amino group and a terminal amino group are cross-linked with disuccinimidyl glutarate (see, e.g., Millward et al., J. Am. Chem. Soc. (2005) 127:14142-14143. In some embodiments, an enzymatic method is used which relies on the reaction between (1) a cysteine and (2) a dehydroalanine or dehydrobutyrine group, catalyzed by a lantibiotic synthetase, to create the thioether bond (see, e.g., Levengood et al., Bioorg. and Med. Chem. Lett. (2008) 18:3025-3028). The dehydro functional group can also be generated chemically by the oxidation of selenium containing amino acid side chains incorporated during translation (see, e.g., Seebeck et al., J. Am. Chem. Soc. 2006).

In some embodiments, bridging moieties comprise an aromatic, 6-membered ring (e.g., benzene). In some embodiments, bridging moieties comprise a heterocyclic, 6-membered ring which includes one nitrogen atoms (e.g., pyridine). In some embodiments, bridging moieties comprise a heterocyclic, 6-membered ring which includes two nitrogen atoms (e.g., pyridazine, pyrimidine, pyrazine). In some embodiments, bridging moieties comprise a heterocyclic, 6-membered ring which includes three nitrogen atoms (e.g., triazanes). In some embodiments, bridging moieties comprise a heterocyclic, 5-membered ring which includes one nitrogen atoms (e.g., pyrrole). In some embodiments, bridging moieties comprise a heterocyclic, 5-membered ring which includes two nitrogen atoms (e.g., imidazole, pyrazole). In some embodiments, bridging moieties comprise a heterocyclic, 5-membered ring which includes three nitrogen atoms (e.g., triazoles).

Peptides of the present disclosure may be cyclized through the carboxy terminus, the amino terminus, or through any other convenient point of attachment, such as, for example, through the sulfur of a cysteine (e.g., through the formation of disulfide bonds between two cysteine residues in a sequence) or any side-chain of an amino acid residue. Further linkages forming cyclic loops may include, but are not limited to, maleimide linkages, amide linkages, ester linkages, ether linkages, thiol ether linkages, hydrazone linkages, or acetamide linkages.

In some embodiments, peptides of the disclosure are formed using a lactam moiety. Such cyclic peptides may be formed, for example, by synthesis on a solid support Wang resin using standard Fmoc chemistry. In some cases, Fmoc-ASP (allyl)-OH and Fmoc-LYS(alloc)-OH are incorporated into peptides to serve as precursor monomers for lactam bridge formation.

In some embodiments, peptides of the present disclosure are linear peptides. In some embodiments, peptides of the present disclosure are cyclic peptides. In some embodiments, the cyclic peptides comprise a disulfide bond. In some embodiments, peptides of the present disclosure are linear peptides prior to the cyclization step. In some embodiments, peptides of the present disclosure are linear peptides prior to the formation of a disulfide bond.

Generally, disulfide bond formation involves a reaction between the sulfhydryl (SH) side chains of two cysteine residues. Proper disulfide bonds provide stability to a protein, decreasing further entropic choices that facilitate folding progression toward the native state by limiting unfolded or improperly folded conformations.

### Terminal Modifications and Conjugations

One method of protecting a peptide from proteolytic degradation involves chemical modification or “capping” of the amino and/or carboxy terminus of the peptides. As used herein, the terms “chemically modified” or “capped” are used interchangeably to refer to the introduction of a blocking group at the end or both ends of the compound by covalent modification. Suitable blocking groups serve to block the ends of the peptides without decreasing the biological activity of the peptides. Any residue located at the amino or carboxy terminus, or both of the described compounds can be chemically modified. In some embodiments, peptides of the present disclosure comprise an N-terminal and/or C-terminal modification.

In one embodiment, the amino end of the compound is chemically modified by acetylation to produce an N-acetylated peptide (which may be represented by “Ac-” in the structure or formula of the present disclosure). In another embodiment, the carboxy terminus of the described peptides is chemically modified by amidation to give the primary carboxamide at the C-terminus (which may be represented as “amide” in the peptide sequence, structure or claims of the present disclosure). In some embodiments, both the amino end and the carboxy end are chemically modified by acetylation and amidation, respectively. However, other capping groups are possible. For example, the amino end can be capped by acylation with groups such as an acetyl group, a benzoyl group, or natural or non-natural amino acids, such as beta-alanine, capped by an acetyl group; or by alkylation with groups such as a benzyl group or a butyl group, or by sulfonylation to produce sulfonamides. Similarly, the carboxy terminus can be esterified or converted to a primary amide, secondary amide and acylsulfonamide or the like.

In some embodiments, the N-terminal capping function is in a linkage to the terminal amino group and may be selected from the group: formyl; alkanoyl, having from 1 to 10 carbon atoms, such as acetyl, propionyl, butyryl; alkenoyl, having from 1 to 10 carbon atoms, such as hex-3-enoyl; alkynoyl, having from 1 to 10 carbon atoms, such as hex-5-ynoyl; aroyl, such as benzoyl or 1-naphthoyl; heteroaroyl, such as 3-pyrroyl or 4-quinoloyl; alkylsulfonyl, such as methanesulfonyl; arylsulfonyl, such as benzenesulfonyl or sulfanilyl; heteroarylsulfonyl, such as pyridine-4-sulfonyl; substituted alkanoyl, having from 1 to 10 carbon atoms, such as 4-aminobutyryl; substituted alkenoyl, having from 1 to 10 carbon atoms, such as 6-hydroxy-hex-3-enoyl; substituted alkynoyl, having from 1 to 10 carbon atoms, such as 3-hydroxy-hex-5-ynoyl; substituted aroyl, such as 4-chlorobenzoyl or 8-hydroxy-naphth-2-oyl; substituted heteroaroyl, such as 2,4-dioxo-1,2,3,4-tetrahydro-3-methyl-quinazolin-6-oyl; substituted alkylsulfonyl, such as 2-aminoethanesulfonyl; substituted arylsulfonyl, such as 5-dimethylamino-1-naphthalenesulfonyl; substituted heteroarylsulfonyl, such as 1-methoxy-6-isoquinolinesulfonyl; carbamoyl or thiocarbamoyl; substituted carbamoyl (R′—NH—CO) or substituted thiocarbamoyl (R′—NH—CS) wherein R′ is alkyl, alkenyl, alkynyl, aryl, heteroaryl, substituted alkyl, substituted alkenyl, substituted alkynyl, substituted aryl, or substituted heteroaryl; substituted carbamoyl (R′—NH—CO) and substituted thiocarbamoyl (R′—NH—CS) wherein R′ is alkanoyl, alkenoyl, alkynoyl, aroyl, heteroaroyl, substituted alkanoyl, substituted alkenoyl, substituted alkynoyl, substituted aroyl, or substituted heteroaroyl, all as above defined; Lys-(Gly) n, where n=1-8; or Tyr-(Gly) n where n=1-8.

In some embodiments, the C-terminal capping function can either be in an amide bond with the terminal carboxyl or in an ester bond with the terminal carboxyl. Capping functions that provide for an amide bond are designated as NR1R2 wherein each R1 and R2 may be independently selected from the following group: hydrogen; alkyl, having from 1 to 10 carbon atoms, such as methyl, ethyl, isopropyl; alkenyl, preferably having from 1 to 10 carbon atoms, such as prop-2-enyl; alkynyl, preferably having from 1 to 10 carbon atoms, such as prop-2-ynyl; substituted alkyl having from 1 to 10 carbon atoms, such as hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl; substituted alkenyl having from 1 to 10 carbon atoms, such as hydroxyalkenyl, alkoxyalkenyl, mercaptoalkenyl, alkylthioalkenyl, halogenoalkenyl, cyanoalkenyl, aminoalkenyl, alkylaminoalkenyl, dialkylaminoalkenyl, alkanoylalkenyl, carboxyalkenyl, carbamoylalkenyl; substituted alkynyl having from 1 to 10 carbon atoms, such as hydroxyalkynyl, alkoxyalkynyl, mercaptoalkynyl, alkylthioalkynyl, halogenoalkynyl, cyanoalkynyl, aminoalkynyl, alkylaminoalkynyl, dialkylaminoalkynyl, alkanoylalkynyl, carboxyalkynyl, carbamoylalkynyl; aroylalkyl having up to 10 carbon atoms, such as phenacyl or 2-benzoylethyl; aryl, such as phenyl or 1-naphthyl; heteroaryl, such as 4-quinolyl; alkanoyl having from 1 to 10 carbon atoms, such as acetyl or butyryl; aroyl, such as benzoyl; heteroaroyl, such as 3-quinoloyl; OR′ or NR′R″ where R′ and R″ each are independently hydrogen, alkyl, aryl, heteroaryl, acyl, aroyl, sulfonyl, sulfinyl, SO₂—R″ or SO—R″ where R″ is substituted or unsubstituted alkyl, aryl, heteroaryl, alkenyl, or alkynyl. In some embodiments, capping functions that provide for an ester bond are designated as OR, wherein R may be: alkoxy; aryloxy; heteroaryloxy; aralkyloxy; heteroaralkyloxy; substituted alkoxy; substituted aryloxy; substituted heteroaryloxy; substituted aralkyloxy; or substituted heteroaralkyloxy.

In some embodiments, peptides of the present disclosure can comprise modifications to the C-terminus of the peptide sequence with one or more of the following moieties: NH₂, NH—CH₃; NH—CH₂—CH₃; NH—CH—(CH₃)₂, NH—CH₂—CH₂—CH₃, NH—CH₂—CH—(CH₃)₂, N(CH₃)₂, N(CH₂—CH₃)₂, or OH.

In some embodiments, peptides of the present disclosure can comprise modifications to the N-terminus of the peptide sequence with one or more peptide-based moieties. In some embodiments, peptides of the present disclosure can comprise modifications to the C-terminus of the peptide sequence with one or more peptide-based moieties. In some embodiments, peptides of the present disclosure can comprise modifications to both the N-terminus of the peptide sequence and the C-terminus of the peptide sequence with one or more peptide-based moieties.

In some embodiments, peptides of the present disclosure can comprise modifications to the N-terminus of the peptide sequence with one or more non-peptide-based moieties. In some embodiments, peptides of the present disclosure can comprise modifications to the C-terminus of the peptide sequence with one or more non-peptide-based moieties. In some embodiments, peptides of the present disclosure can comprise modifications to both the N-terminus of the peptide sequence and the C-terminus of the peptide sequence with one or more non-peptide-based moieties.

In some embodiments, peptides of the present disclosure can comprise modifications to the N-terminus which comprise a string of 5 or 6 Glu amino acids. In some embodiments, peptides of the present disclosure can comprise modifications to the N-terminus which comprise a string of 5 or 6 Lys amino acids. In some embodiments, peptides of the present disclosure can comprise modifications to the N-terminus which comprise a string of 5 or 6 amino acids, each independently selected from Glu or Lys.

In some embodiments, peptides of the present disclosure comprise an N-terminal peptide consisting of a chain of about 15 to about 400 identical amino acids. In some embodiments, the N-terminal peptide comprises about 25 to about 300 identical amino acids, about 50 to about 200 identical amino acids, about 75 to about 150 identical amino acids, about 90 to about 120 identical amino acids, or about 100 or 110 identical amino acids. In some embodiments, the N-terminal peptide comprises: poly(glutamic acid) peptides (PGa), poly(aspartic acid) peptides (PAS), poly(lysine) peptides (PLy), poly(arginine) peptides (PAr), poly(histidine) peptides (PHi), poly(ornithine) peptides (POr), or combinations thereof.

In some embodiments, peptides of the present disclosure comprise an N-terminal modification. In a further embodiment, the N-terminal modification comprises an N-terminal acetyl group (represented as Ac). In some embodiments, peptides of the present disclosure comprise a C-terminal modification. In a further embodiment, the C-terminal modification comprises an amide group (represented as amide or CONH2).

Peptides of the disclosure may be peptidomimetics. A “peptidomimetic” or “peptide mimetic” is a peptide in which the molecule contains structural elements that are not found in natural peptides (i.e., peptides comprised of only the 20 proteinogenic amino acids). In some embodiments, peptidomimetics are capable of recapitulating or mimicking the biological action(s) of a natural peptide. A peptidomimetic may differ in many ways from natural peptides, for example through changes in backbone structure or through the presence of amino acids that do not occur in nature. In some cases, peptidomimetics may include amino acids with side chains that are not found among the known 20 proteinogenic amino acids; non-peptide-based bridging moieties used to effect cyclization between the ends or internal portions of the molecule; substitutions of the amide bond hydrogen moiety by methyl groups (N-methylation) or other alkyl groups; substitutions of the amino acid alpha hydrogen moiety by methyl groups (alpha-methylation) or other alkyl groups; replacement of a peptide bond with a chemical group or bond that is resistant to chemical or enzymatic treatments; N- and C-terminal modifications; and/or conjugation with a non-peptidic extension (such as polyethylene glycol, lipids, carbohydrates, nucleosides, nucleotides, nucleoside bases, various small molecules, or phosphate or sulfate groups).

As used herein, the term “amino acid” includes the residues of the natural amino acids as well as unnatural amino acids. The 20 natural proteinogenic amino acids are identified and referred to herein by either the one-letter or three-letter designations as follows: aspartic acid (Asp: D), isoleucine (Ile: I), threonine (Thr: T), leucine (Leu: L), serine (Ser: S), tyrosine (Tyr: Y), glutamic acid (Glu: E), phenylalanine (Phe: F), proline (Pro: P), histidine (His: H), glycine (Gly: G), lysine (Lys: K), alanine (Ala: A), arginine (Arg: R), cysteine (Cys: C), tryptophan (Trp: W), valine (Val: V), glutamine (Gln: Q) methionine (Met: M), asparagine (Asn: N). Naturally occurring amino acids exist in their levorotary (L) stereoisomeric forms. Amino acids referred to herein are L-stereoisomers except where otherwise indicated. The term “amino acid” also includes amino acids bearing a conventional amino protecting group (e.g. acetyl or benzyloxycarbonyl), as well as natural and unnatural amino acids protected at the carboxy terminus (e.g., as a (C1-C6)alkyl, phenyl or benzyl ester or amide; or as an alpha-methylbenzyl amide). Other suitable amino and carboxy protecting groups are known to those skilled in the art (See for example, Greene, T. W.; Wutz, P. G. M., Protecting Groups In Organic Synthesis; second edition, 1991, New York, John Wiley & sons, Inc., and documents cited therein, the contents of each of which are herein incorporated by reference in their entirety). Peptides and/or peptide compositions of the present disclosure may also include modified amino acids.

“Unnatural” amino acids have side chains or other features not present in the 20 naturally-occurring amino acids listed above and include, but are not limited to: N-methyl amino acids, N-alkyl amino acids, alpha, alpha substituted amino acids, beta-amino acids, alpha-hydroxy amino acids, D-amino acids, and other unnatural amino acids known in the art (See, e.g., Josephson et al., J. Am. Chem. Soc. (2005) 127: 11727-11735; Forster, A. C. et al. Proc. Natl. Acad. Sci. USA (2003) 100:6353-6357; Subtelny et al., J. Am. Chem. Soc. (2008) 130: 6131-6136; Hartman, M. C. T. et al. PLOS ONE (2007)₂:e972; and Hartman et al., Proc. Natl. Acad. Sci. USA (2006) 103: 4356-4361). Further unnatural amino acids useful for the optimization of peptides and/or peptide compositions of the present disclosure include, but are not limited to 1,2,3,4-tetrahydroisoquinoline-1-carboxylic acid, 1-amino-2,3-hydro-1H-indene-1-carboxylic acid, homolysine, homoarginine, homoserine, 2-aminoadipic acid, 3-aminoadipic acid, beta-alanine, aminopropionic acid, 2-aminobutyric acid, 4-aminobutyric acid, 5-aminopentanoic acid, 5-afminohexanoic acid, 6-aminocaproic acid, 2-aminoheptanoic acid, 2-aminoisobutyric acid, 3-aminoisobutyric acid, 2-aminopimelic acid, desmosine, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylpentylglycine, naphthylalanine, ornithine, pentylglycine, thioproline, norvaline, tert-butylglycine (also known as tert-leucine), phenylglycine, azatryptophan, 5-azatryptophan, 7-azatryptophan, 4-fluorophenylalanine, penicillamine, sarcosine, homocysteine, 1-aminocyclopropanecarboxylic acid, 1-aminocyclobutanecarboxylic acid, 1-aminocyclopentanecarboxylic acid, 1-aminocyclohexanecarboxylic acid, 4-aminotetrahydro-2H-pyran-4-carboxylic acid, (S)-2-amino-3-(1H-tetrazol-5-yl)propanoic acid, cyclopentylglycine, cyclohexylglycine, cyclopropylglycine, η-ω-methyl-arginine, 4-chlorophenylalanine, 3-chlorotyrosine, 3-fluorotyrosine, 5-fluorotryptophan, 5-chlorotryptophan, citrulline, 4-chloro-homophenylalanine, homophenylalanine, 4-aminomethyl-phenylalanine, 3-aminomethyl-phenylalanine, octylglycine, norleucine, tranexamic acid, 2-amino pentanoic acid, 2-amino hexanoic acid, 2-amino heptanoic acid, 2-amino octanoic acid, 2-amino nonanoic acid, 2-amino decanoic acid, 2-amino undecanoic acid, 2-amino dodecanoic acid, aminovaleric acid, and 2-(2-aminoethoxy)acetic acid, pipecolic acid, 2-carboxy azetidine, hexafluoroleucine, 3-Fluorovaline, 2-amino-4,4-difluoro-3-methylbutanoic acid, 3-fluoro-isoleucine, 4-fluoroisoleucine, 5-fluoroisoleucine, 4-methyl-phenylglycine, 4-ethyl-phenylglycine, 4-isopropyl-phenylglycine, (S)-2-amino-5-azidopentanoic acid (also referred to herein as “X02”), (S)-2-aminohept-6-enoic acid (also referred to herein as “X30”), (S)-2-aminopent-4-ynoic acid (also referred to herein as “X31”), (S)-2-aminopent-4-enoic acid (also referred to herein as “X12”), (S)-2-amino-5-(3-methylguanidino) pentanoic acid, (S)-2-amino-3-(4-(aminomethyl)phenyl)propanoic acid, (S)-2-amino-3-(3-(aminomethyl)phenyl)propanoic acid, (S)-2-amino-4-(2-aminobenzo[d]oxazol-5-yl)butanoic acid, (S)-leucinol, (S)-valinol, (S)-tert-leucinol, (R)-3-methylbutan-2-amine, (S)-2-methyl-1-phenylpropan-1-amine, and (S)—N,2-dimethyl-1-(pyridin-2-yl)propan-1-amine, (S)-2-amino-3-(oxazol-2-yl)propanoic acid, (S)-2-amino-3-(oxazol-5-yl)propanoic acid, (S)-2-amino-3-(1,3,4-oxadiazol-2-yl)propanoic acid, (S)-2-amino-3-(1,2,4-oxadiazol-3-yl)propanoic acid, (S)-2-amino-3-(5-fluoro-1H-indazol-3-yl)propanoic acid, and (S)-2-amino-3-(1H-indazol-3-yl)propanoic acid, (S)-2-amino-3-(oxazol-2-yl)butanoic acid, (S)-2-amino-3-(oxazol-5-yl) butanoic acid, (S)-2-amino-3-(1,3,4-oxadiazol-2-yl)butanoic acid, (S)-2-amino-3-(1,2,4-oxadiazol-3-yl)butanoic acid, (S)-2-amino-3-(5-fluoro-1H-indazol-3-yl)butanoic acid, and (S)-2-amino-3-(1H-indazol-3-yl)butanoic acid, 2-(2′MeOphenyl)-2-amino acetic acid, tetrahydro 3-isoquinolinecarboxylic acid and stereoisomers thereof (including, but not limited, to D and L isomers). Additional unnatural amino acids useful for the optimization of peptides and/or peptide compositions of the present disclosure include, but are not limited to (S)-3-(1-acetylpiperidin-4-yl)-2-aminopropanoic acid, (S)-4-(4-(2-amino-2-carboxyethyl) piperidin-1-yl)-4-oxobutanoic acid, (S)-2-amino-3-(1-(carboxymethyl) piperidin-4-yl)propanoic acid, (S)-2-amino-3-(1-carbamoylpiperidin-4-yl)propanoic acid and stereoisomers thereof (including, but not limited, to D and L isomers).

Additional unnatural amino acids that are useful in the optimization of peptides or peptide compositions of the disclosure include but are not limited to halogenated amino acids wherein one or more carbon bound hydrogen atoms are replaced by one or more halogen atoms. The number of halogen atoms included can range from 1 up to and including all of the hydrogen atoms.

In some embodiments, unnatural amino acids that are useful in the optimization of peptides or peptide compositions of the disclosure include but are not limited to fluorinated amino acids wherein one or more carbon bound hydrogen atoms are replaced by one or more fluorine atoms. The number of fluorine atoms included can range from 1 up to and including all of the hydrogen atoms. Examples of such amino acids include but are not limited to 3-fluoroproline, 3,3-difluoroproline, 4-fluoroproline, 4,4-difluoroproline, 3,4-difluroproline, 3,3,4,4-tetrafluoroproline, 4-fluorotryptophan, 5-flurotryptophan, 6-fluorotryptophan, 7-fluorotryptophan, and stereoisomers thereof.

In some embodiments, unnatural amino acids that are useful in the optimization of peptides or peptide compositions of the disclosure include but are not limited to chlorinated amino acids wherein one or more carbon bound hydrogen atoms are replaced by one or more chlorine atoms. The number of chlorine atoms included can range from 1 up to and including all of the hydrogen atoms.

Further unnatural amino acids that are useful in the optimization of peptides of the disclosure include but are not limited to those that are disubstituted at the α-carbon. These include amino acids in which the two substituents on the α-carbon are the same, for example a-amino isobutyric acid, and 2-amino-2-ethyl butanoic acid, as well as those where the substituents are different, for example α-methylphenylglycine and α-methylproline. Further the substituents on the α-carbon may be taken together to form a ring, for example 1-aminocyclopentanecarboxylic acid, 1-aminocyclobutanecarboxylic acid, 1-aminocyclohexanecarboxylic acid, 3-aminotetrahydrofuran-3-carboxylic acid, 3-aminotetrahydropyran-3-carboxylic acid, 4-aminotetrahydropyran-4-carboxylic acid, 3-aminopyrrolidine-3-carboxylic acid, 3-aminopiperidine-3-carboxylic acid, 4-aminopiperidine-4-carboxylix acid, and stereoisomers thereof.

Additional unnatural amino acids that are useful in the optimization of peptides or peptide compositions of the disclosure include but are not limited to analogs of tryptophan in which the indole ring system is replaced by another 9 or 10 membered bicyclic ring system comprising 0, 1, 2, 3 or 4 heteroatoms independently selected from N, O, or S. Each ring system may be saturated, partially unsaturated, or fully unsaturated. The ring system may be substituted by 0, 1, 2, 3, or 4 substituents at any substitutable atom. Each substituent may be independently selected from H, F, Cl, Br, CN, COOR, CONRR′, oxo, OR, NRR′. Each R and R′ may be independently selected from H, C1-C20 alkyl, or C1-C20 alkyl-O-C1-20 alkyl.

In some embodiments, analogs of tryptophan (also referred to herein as “tryptophan analogs”) may be useful in the optimization of peptides or peptide compositions of the disclosure. Tryptophan analogs may include, but are not limited to 5-fluorotryptophan [(5-F)W], 5-methyl-O-tryptophan [(5-MeO)W], 1-methyltryptophan [(1-Me-W) or (1-Me)W], D-tryptophan (D-Trp), azatryptophan (including, but not limited to 4-azatryptophan, 7-azatryptophan and 5-azatryptophan) 5-chlorotryptophan, 4-fluorotryptophan, 6-fluorotryptophan, 7-fluorotryptophan, and stereoisomers thereof. Except where indicated to the contrary, the term “azatryptophan” and its abbreviation, “azaTrp,” as used herein, refer to 7-azatryptophan.

Modified amino acid residues useful for the optimization of peptides and/or peptide compositions of the present disclosure include, but are not limited to those which are chemically blocked (reversibly or irreversibly); chemically modified on their N-terminal amino group or their side chain groups; chemically modified in the amide backbone, as for example, N-methylated, D (unnatural amino acids) and L (natural amino acids) stereoisomers; or residues wherein the side chain functional groups are chemically modified to another functional group. In some embodiments, modified amino acids include without limitation, methionine sulfoxide; methionine sulfone; aspartic acid-(beta-methyl ester), a modified amino acid of aspartic acid; N-ethylglycine, a modified amino acid of glycine; alanine carboxamide; and/or a modified amino acid of alanine. Unnatural amino acids may be purchased from Sigma-Aldrich (St. Louis, MO), Bachem (Torrance, CA) or other suppliers. Unnatural amino acids may further include any of those listed in Table 2 of US patent publication US 2011/0172126, the contents of which are incorporated herein by reference in their entirety.

In some embodiments, amino acids for use in the present disclosure are modified using an organic proteinaceous or non-proteinaceous derivatizing agent. In some embodiments, amino acids for use in the present disclosure are modified using post-translational modification. In some embodiments, modifications are introduced by reacting targeted amino acid residues of the peptide with an organic derivatizing agent that is capable of reacting with selected side-chains or terminal residues. In some embodiments, modifications are introduced by harnessing mechanisms of post-translational modifications that function in selected recombinant host cells. Certain post-translational modifications are the result of the action of recombinant host cells on an expressed peptide. As one examples, glutaminyl and asparaginyl residues are frequently post-translationally deamidated to the corresponding glutamyl and aspartyl residues under certain post-translational conditions (e.g., under mildly acidic conditions). Other post-translational modifications include: hydroxylation of proline and lysine; phosphorylation of hydroxyl groups of tyrosinyl, seryl or threonyl residues; and methylation of the alpha-amino groups of lysine, arginine, and histidine side chains (see, e.g, Creighton et al., Proteins: Structure and Molecular Properties, W.H. Freeman & Co., San Francisco, 1983, pp. 79-86).

In some embodiments, amino acid modifications include the bonding of non-proteinaceous polymers to peptides of the present disclosure. Examples of non-proteinaceous polymers include hydrophilic synthetic polymers (i.e., non-natural polymers), such as hydrophilic polyvinyl polymers (e.g., polyvinylalcohol and polyvinylpyrrolidone). The Examples of non-proteinaceous polymers also include polyethylene glycol, polypropylene glycol and polyoxyalkylenes. In some embodiments, amino acid modifications include the bonding of non-proteinaceous polymers to peptides of the present disclosure, as described in U.S. Pat. Nos. 4,640,835, 4,496,689, 4,301,144, 4,670,417, 4,791,192, and 4,179,337; the contents of which are each incorporated herein by reference in their entirety, as related to amino acid modifications for us in the present disclosure.

4lg-B7-H3 Epitope

The peptides of the disclosure that bind to human 4lg-B7-H3 bind to specific amino acids in 4lg-B7-H3, i.e., a 4lg-B7-H3 epitope. As used herein, the term “epitope” refers to the specific portion(s) of a target (e.g., 4lg-B7-H3) which interact (e.g., bind) with a binding entity (e.g., the peptides of the disclosure). In an embodiment, the cyclic peptide has binding specificity for B7-H3 isoform 4lg (4lg-B7-H3). In another embodiment, the cyclic peptide has an affinity (K_{D}) for B7-H3 isoform 2lg (2lg-B7-H3) of about 10,000 nM or weaker. In yet another embodiment, the cyclic peptide has an affinity (K_{D}) for B7-H3 isoform 21 g (2lg-B7-H3) of about 1,000 nM or weaker. In still another embodiment, the cyclic peptide has an affinity (K_{D}) for 2lg-B7-H3 of about 10,000 nM to about 50,000 nM. In an embodiment, the cyclic peptide has an affinity (K_{D}) for 2lg-B7-H3 of about 1000 nM to about 10,000 nM. In still another embodiment, the cyclic peptide lacks detectable affinity (K_{D}) for 2lg-B7-H3. In an embodiment, the cyclic peptide has an affinity (K_{D}) for 4lg-B7-H3 of about 500 nM or stronger. In another embodiment, the cyclic peptide has an affinity (K_{D}) for 4lg-B7-H3 of about 500 nM to about 1 nM. In yet another embodiment, the cyclic peptide has an affinity (K_{D}) for 4lg-B7-H3 of about 2-fold to about 1,000-fold stronger than an affinity (K_{D}) for 2lg-B7-H3. In still another embodiment, the cyclic peptide has an affinity (K_{D}) for 4lg-B7-H3 of about 5-fold to about 1,000-fold stronger than an affinity (K_{D}) for 2lg-B7-H3. In some embodiments, the peptides having binding specificity for 4lg-B7-H3 bind to one or more amino acids of D154, Q286, K291, M147, S234, T236, T238, and T290 of a 4lg-B7-H3 amino acid sequence of SEQ ID NO: 553.

In some embodiments, the peptides bind to amino acids D154, Q286, K291, M147, S234, T236, T238, and T290 of a 4lg-B7-H3 amino acid sequence of SEQ ID NO: 553. In an embodiment, the cyclic peptide binds to amino acids D154, Q286, K291, M147, S234, T236, T238, and T290 of a 4lg-B7-H3 amino acid sequence of SEQ ID NO: 553. In another embodiment, the cyclic peptide comprises an amino acid sequence of LYCVGKADVYCWFVE, or a derivative thereof comprising one or more unnatural amino acids. In yet another embodiment, amino acids Y₂, K6, D8, V9, Y10, C11, and E15 bind to 4lg-B7-H3.

In still another embodiment, the peptides are capable of binding 4lg-B7-H3 with an affinity K_{D} value of about 1×10⁻⁸ M to about 1×10⁻¹² M. In an embodiment, the peptides are capable of binding 4lg-B7-H3 with an affinity K_{D} value of about 1×10⁻⁸ M to about 1×10⁻¹⁰ M.

In an aspect, provided herein is a method of inhibiting the activity of B7-H3 isoform 4lg (4lg-B7-H3) on a cell, comprising contacting a cell with a cyclic peptide having binding specificity for 4lg-B7-H3, thereby inhibiting the activity of 4lg-B7-H3 on the cell. In another aspect, provided herein is a method of selectively inhibiting the activity of B7-H3 isoform 41 g (4lg-B7-H3) relative to the activity of B7-H3 isoform 2lg (2lg-B7-H3) on a cell, comprising contacting a cell with a cyclic peptide having binding specificity for 4lg-B7-H3, thereby inhibiting the activity of 4lg-B7-H3 on the cell.

In an embodiment, the peptide is a cyclic peptide of the present disclosure.

### Synthesis of Peptides

The present disclosure presents methods of synthesizing peptides and compounds of the present disclosure. In some embodiments, peptides of the present disclosure can be obtained by inducing the formation of a covalent bond between an amino group at the N-terminus of a peptide (if provided), and a carboxyl group of a reactive amino acid side chain moiety (if provided). In some embodiments, peptides and compounds of the present disclosure can be synthesized by any known conventional procedure for the formation of a peptide linkage between amino acids. Such conventional procedures include, for example, any solution phase procedure permitting a condensation between the free alpha amino group of an amino acid or residue thereof (having its carboxyl group or other reactive groups protected) and the free primary carboxyl group of another amino acid or residue thereof (having its amino group or other reactive groups protected). In some embodiments, the peptides of the present disclosure may be synthesized by solid-phase synthesis and purified according to methods known in the art. Any of a number of well-known procedures utilizing a variety of resins and reagents may be used to prepare the peptides of the present disclosure.

In some embodiments, the process for synthesizing peptides may be carried out by a procedure whereby each amino acid in the desired sequence is added one at a time in succession to another amino acid or residue thereof. In some embodiments, the process for synthesizing peptides may be carried out by a procedure whereby multiple peptide fragments with portions of the desired amino acid sequence are first synthesized, and then condensed to provide the desired peptide sequence.

In some embodiments, the process for synthesizing peptides may be carried out using solid phase peptide synthesis, which includes methods well known and practiced in the art (e.g., Symphony Multiplex Peptide Synthesizer (Rainin Instrument Company) automated peptide synthesizer). In some embodiments, the process for synthesizing peptides may be carried using standard Fmoc methodology on an automated synthesizer (e.g., Advanced ChemTech 440M05, Louisville, Ky). In some embodiments, the process for synthesizing peptides may be carried using coupling reagents such as 2-(1-H-Benzotriazol-1-yl)-1,1,3,3-tetramethyluronium hexafluorophosphate (HBTU) and/or 1-Hydroxybenzotriazole (HOBt).

Solid phase peptide synthesis can be carried out by sequentially incorporating the desired amino acid residues one at a time into the growing side chain according to the general principles of solid phase methods. These methods are disclosed in numerous references, including Merrifield, et al., Solid phase synthesis (Nobel lecture), Angew Chem (1985) 24:799-810; Barany et al., The Peptides, Analysis, Synthesis and Biology, Vol. 2; Gross et al., Eds. Academic Press 1-284 (1980), the contents of which are each incorporated herein by reference in their entirety, as related to processes and protocols for synthesizing peptides.

Solid phase synthesis of the peptide is generally commenced from the C-terminal end of the peptide by coupling a protected alpha amino acid to a suitable resin. Examples of known methods for preparing substituted amide derivatives on solid-phase have been described in the art (see, e.g., Barn D. R. et al., Tetrahedron Letters (1996), 37:3213-3216; DeGrado et al., J. Org. Chem., (1982) 47:3258-3261; the contents of which are each incorporated herein by reference in their entirety as related to methods and systems for solid-phased peptide synthesis). As an example, starting materials can be prepared by attaching an alpha amino-protected amino acid by an ester linkage to a p-benzyloxybenzyl alcohol (Wang) resin or an oxime resin by well-known means. The peptide chain is grown with the desired sequence of amino acids, and the peptide-resin is then treated with a solution of appropriate amine (such as methyl amine, dimethyl amine, ethylamine, and so on). Peptides employing a p-benzyloxybenzyl alcohol (Wang) resin may be cleaved from the resin by aluminum chloride in DCM, and peptides employing an oxime resin may be cleaved by DCM.

In some embodiments, reactive side chain groups of the various amino acid residues are protected with suitable protecting groups, which prevent a chemical reaction from occurring at that site until the protecting group is removed. In some embodiments, the alpha amino group of an amino acid residue or fragment is protected while that entity reacts at the carboxyl group, followed by the selective removal of the alpha amino protecting group to allow a subsequent reaction to take place at that site. Examples of protecting groups for use in the present disclosure have been disclosed and are known in solid phase synthesis methods and solution phase synthesis methods.

In some embodiments, alpha amino groups may be protected by a suitable protecting group, including: a urethane-type protecting group, such as benzyloxycarbonyl (Z) and substituted benzyloxycarbonyl, such as p-chlorobenzyloxycarbonyl, P-nitrobenzyloxycarbonyl, p-bromobenzyloxycarbonyl, p-biphenyl-isopropoxycarbonyl, 9-fluorenylmethoxycarbonyl (Fmoc) and p-methoxybenzyloxycarbonyl (Moz); aliphatic urethane-type protecting groups, such as t-butyloxycarbonyl (Boc), diisopropylmethoxycarbonyl, isopropoxycarbonyl, and allyloxycarbonyl.

In some embodiments, guanidino amino groups (such as those found in arginine) may be protected by a suitable protecting group, such as nitro, p-toluenesulfonyl (Tos), Z, pentamethylchromanesulfonyl (Pmc), adamantyloxycarbonyl, pentamethyldihydrobenzofuran-5-sulfonyl (Pbf) and Boc.

As a non-limiting example, solid phase synthesis of a peptide can be commenced from the C-terminal end of the peptide by coupling a protected alpha amino acid to a suitable resin. The starting material can be prepared by attaching an alpha amino-protected amino acid by an ester linkage to a p-benzyloxybenzyl alcohol (Wang) resin, a 2-chlorotrityl chloride resin or an oxime resin, by an amide bond between an Fmoc-Linker, such as p-[(R,S)-α-[1-(9H-fluor-en-9-yl)-methoxyformamido]-2,4-dimethyloxybenzyl]-phenoxyacetic acid (Rink linker) to a benzhydrylamine (BHA) resin, or by other means well known in the art. Fmoc-Linker-BHA resin supports are commercially available and generally used when feasible. The resins are then carried through repetitive addition cycles as necessary to add amino acids sequentially. The alpha amino Fmoc protecting groups are then removed under basic conditions (e.g., piperidine, piperazine, diethylamine, or morpholine (20-40% v/v) in N,N-dimethylformamide (DMF)). Following removal of the alpha amino protecting group, the subsequent protected amino acids are coupled stepwise in the desired order to obtain an intermediate, protected peptide-resin. The activating reagents used for coupling of the amino acids in the solid phase synthesis of the peptides are well known in the art. After the peptide is synthesized, if desired, the orthogonally protected side chain protecting groups may be removed using methods well known in the art for further derivatization of the peptide.

Reactive groups in a peptide can be selectively modified, either during solid phase synthesis or after removal from the resin. For example, peptides can be modified to obtain N-terminus modifications, such as acetylation, while on resin, or may be removed from the resin by use of a cleaving reagent and then modified. Similarly, methods for modifying side chains of amino acids are well known to those skilled in the art of peptide synthesis. The choice of modifications made to reactive groups present on the peptide will be determined, in part, by the characteristics that are desired in the peptide.

In some embodiments, the N-terminus group is modified by introduction of an N-acetyl group. As a non-limiting example, the peptide synthesis can include a step wherein, after removal of the protecting group at the N-terminal, a resin-bound peptide is reacted with acetic anhydride in dichloromethane in the presence of an organic base, such as diisopropylethylamine. Other methods of N-terminus acetylation are known in the art, including solution phase acetylation.

In some embodiments, peptides of the present disclosure can comprise cyclic peptides having one or more bridging moieties (e.g., cyclic structure, staple, bridge, etc.).

In some embodiments, the peptide can be synthesized using solid phase peptide synthesis, and then cyclized prior to cleavage from the peptide resin. If the peptide is being cyclized through reactive side chain moieties, the desired side chains are first deprotected under specific deprotection conditions in a suitable solvent, and a cyclic coupling agent is then added. Suitable solvents include, but are not limited to: DMF, dichloromethane (DCM), and 1-methyl-2-pyrrolidone (NMP). Suitable cyclic coupling reagents include, but are not limited to: 2-(1H-benzotriazol-1-yl)-1,1,3,3-tetramethyluronium tetrafluoroborate (TBTU), 2-(1H-benzotriazol-1-yl)-1,1,3,3-tetramethyluronium hexafluorophosphate (HBTU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP), 2-(7-aza-1H-benzotriazol-1-yl)-1,1,3,3-tetramethyluronium tetrafluoroborate (TATU), 2-(2-oxo-1(2H)-pyridyl)-1,1,3,3-tetramethyluronium tetrafluoroborate (TPTU), and N,N′-dicyclohexylcarbodiimide/1-hydroxybenzotriazole (DCCI/HOBt). In some embodiments, coupling of the cyclic moiety to the peptide chain is initiated by use of a suitable base, such as N,N-diisopropylethylamine (DIPEA), sym-collidine, or N-methylmorpholine (NMM).

The cyclized peptides can then be cleaved from the solid phase using any suitable reagent, such as ethylamine in DCM. The resulting crude peptide is dried, and remaining amino acid side chain protecting groups (if any) are cleaved using suitable reagents, such as trifluoroacetic acid (TFA) in the presence of water and 1,2-ethanedithiol (EDT). The final product is precipitated by adding cold ether and collected by filtration. Final purification can be by reverse phase high performance liquid chromatography (RP-HPLC), using a suitable column, such as a C18 column. Other methods of separation or purification, such as methods based on the size or charge of the peptide, can also be employed. Once purified, the peptide can be characterized by any number of methods, such as high-performance liquid chromatography (HPLC), amino acid analysis, mass spectrometry, and the like.

In some embodiments, peptides of the present disclosure can comprise one or more modifications (e.g., substitution, addition, deletion) to one or more terminus (e.g., N-terminus, C-terminus, or both) of the peptide sequence. In some embodiments, terminus-modified peptides can be synthesized using solid phase peptide synthesis, and then modified prior to cleavage from the peptide resin.

The present disclosure contemplates variants and derivatives of peptides presented herein. These include substitutional, insertional, deletional, and covalent variants and derivatives. As used herein, the term “derivative” is used synonymously with the term “variant” and refers to a molecule that has been modified or changed in any way relative to a reference molecule or starting molecule.

In one embodiment, the peptides described herein comprise replacement of one or more L-amino acid residues with one or more D-amino acid residues. This embodiment is believed to increase proteolytic stability by steric hindrance and by a propensity of D-amino acids to stabilise β-turn conformations (Tugyi et al, PNAS (2005), 102(2), 413-418).

In some embodiments, peptides of the present disclosure may be in the salt forms. The salts of the peptides can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

In some embodiments, salts of the present disclosure may be salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. In some embodiments, the salt may be the hydrochloride salt. In some embodiments, the salt may be the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides contain an amine function, these may form quaternary ammonium salts, for example by reaction with an alkylating agent according to methods well known to the skilled person.

The peptides disclosed herein, may bind to a target receptor with an equilibrium dissociation constant (K_{D}) of from about 0.001 nM to about 0.01 nM, from about 0.005 nM to about 0.05 nM, from about 0.01 nM to about 0.1 nM, from about 0.05 nM to about 0.5 nM, from about 0.1 nM to about 1.0 nM, from about 0.5 nM to about 5.0 nM, from about 2 nM to about 10 nM, from about 8 nM to about 20 nM, from about 15 nM to about 45 nM, from about 30 nM to about 60 nM, from about 40 nM to about 80 nM, from about 50 nM to about 100 nM, from about 75 nM to about 150 nM, from about 100 nM to about 500 nM, from about 200 nM to about 800 nM, from about 400 nM to about 1,000 nM or at least 1,000 nM.

In some embodiments, the peptides disclosed herein, may bind to B7-H3 with an equilibrium dissociation constant (K_{D}) of from about 0.001 nM to about 0.01 nM, from about 0.005 nM to about 0.05 nM, from about 0.01 nM to about 0.1 nM, from about 0.05 nM to about 0.5 nM, from about 0.1 nM to about 1.0 nM, from about 0.5 nM to about 5.0 nM, from about 2 nM to about 10 nM, from about 8 nM to about 20 nM, from about 15 nM to about 45 nM, from about 30 nM to about 60 nM, from about 40 nM to about 80 nM, from about 50 nM to about 100 nM, from about 75 nM to about 150 nM, from about 100 nM to about 500 nM, from about 200 nM to about 800 nM, from about 400 nM to about 1,000 nM or at least 1,000 nM.

### Chelating Agents

Chelating agents (CAs) may include metal chelating agents that associate with metal cargo (e.g., metallic nuclide cargo). Chelating agents may include macromolecular compounds. In some embodiments, chelating agents include acyclic or macrocyclic compounds.

Exemplary chelating agents (also referred to as “Chelators”) are given below in Table

TABLE 3
Exemplary chelating agents.

| Name | |
|---|---|
| (Desig- | |
| nation) | Structure |
| DOTA | 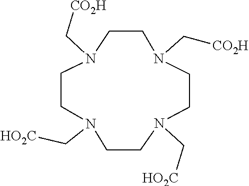 [SMILES: O=C(O)CN1CCN(CC(=O)O)CCN(CC(=O)O)CCN(CC(=O)O)CC1] |
| DO3A | 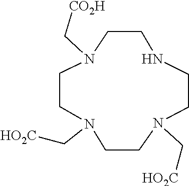 [SMILES: O=C(O)CN1CCNCCN(CC(=O)O)CCN(CC(=O)O)CC1] |
| C-DOTA | 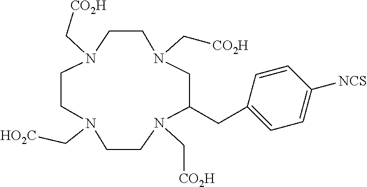 |
| PA-DOTA | 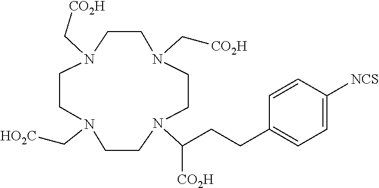 |
| DODASA | 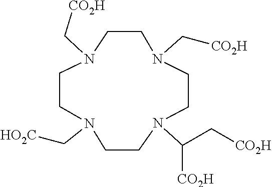 [SMILES: O=C(O)CC(C(=O)O)N1CCN(CC(=O)O)CCN(CC(=O)O)CCN(CC(=O)O)CC1] |
| C4amino- DOTA | 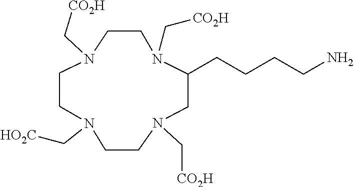 [SMILES: NCCCCC1CN(CC(=O)O)CCN(CC(=O)O)CCN(CC(=O)O)CCN1CC(=O)O] |
| DOTAGA | 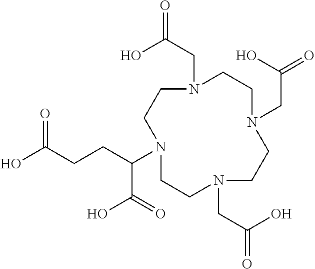 [SMILES: O=C(O)CCC(C(=O)O)N1CCN(CC(=O)O)CCN(CC(=O)O)CCN(CC(=O)O)CC1] |
| DOTASA | 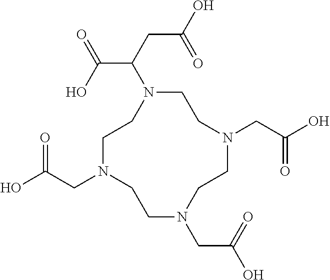 [SMILES: O=C(O)CC(C(=O)O)N1CCN(CC(=O)O)CCN(CC(=O)O)CCN(CC(=O)O)CC1] |
| DTPA | 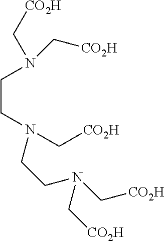 [SMILES: O=C(O)CN(CCN(CC(=O)O)CC(=O)O)CCN(CC(=O)O)CC(=O)O] |
| CHX-A″- DTPA | 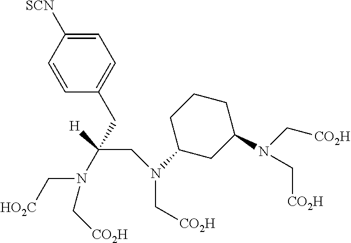 |
| ca-DTPA | 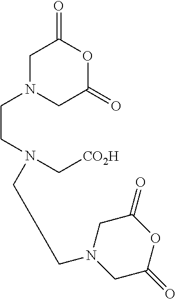 [SMILES: O=C(O)CN(CCN1CC(=O)OC(=O)C1)CCN1CC(=O)OC(=O)C1] |
| ibca-DTPA | 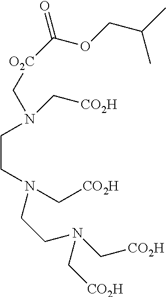 |
| 1B4M-DTPA | 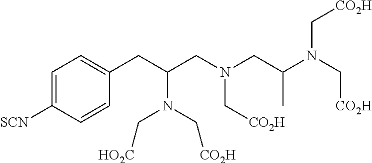 |
| lys-DTPA | 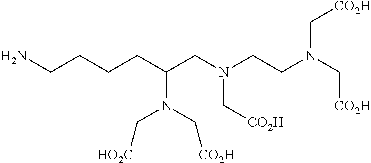 [SMILES: NCCCCC(CN(CCN(CC(=O)O)CC(=O)O)CC(=O)O)N(CC(=O)O)CC(=O)O] |
| vinyl DTPA | 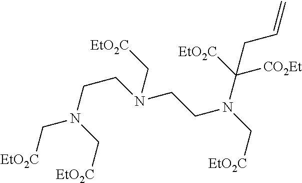 [SMILES: C=CCC(C(=O)OCC)(C(=O)OCC)N(CCN(CCN(CC(=O)OCC)CC(=O)OCC)CC(=O)OCC)CC(=O)OCC] |
| glu-DTPA | 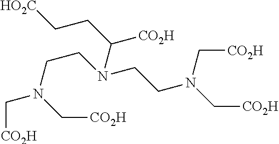 [SMILES: O=C(O)CCC(C(=O)O)N(CCN(CC(=O)O)CC(=O)O)CCN(CC(=O)O)CC(=O)O] |
| NOTA | 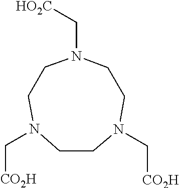 [SMILES: O=C(O)CN1CCN(CC(=O)O)CCN(CC(=O)O)CC1] |
| p-NCS-Bz- NOTA | 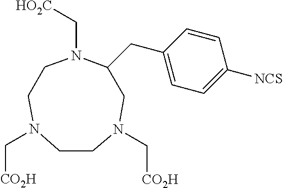 |
| N-NOTA | 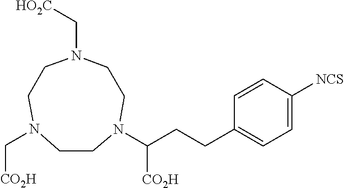 |
| NODAGA | 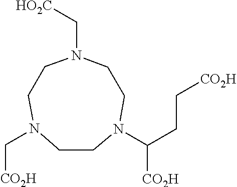 [SMILES: O=C(O)CCC(C(=O)O)N1CCN(CC(=O)O)CCN(CC(=O)O)CC1] |
| NODASA | 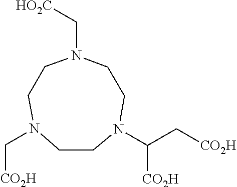 [SMILES: O=C(O)CC(C(=O)O)N1CCN(CC(=O)O)CCN(CC(=O)O)CC1] |
| triazacyclo- nonane-TM | 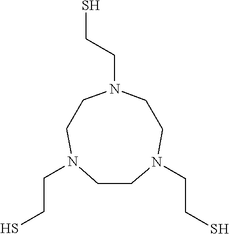 [SMILES: SCCN1CCN(CCS)CCN(CCS)CC1] |
| NOTP | 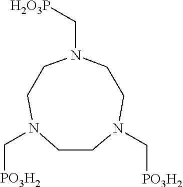 |
| HYNIC | 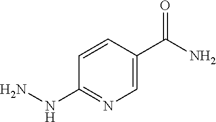 [SMILES: NNc1ccc(C(N)=O)cn1] |
| EDTA | 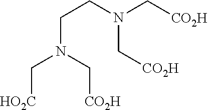 [SMILES: O=C(O)CN(CCN(CC(=O)O)CC(=O)O)CC(=O)O] |
| Deferox- amine (DFO) | 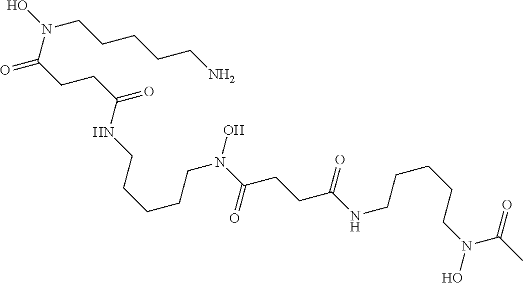 [SMILES: CC(=O)N(O)CCCCCNC(=O)CCC(=O)N(O)CCCCCNC(=O)CCC(=O)N(O)CCCCCN] |
| | 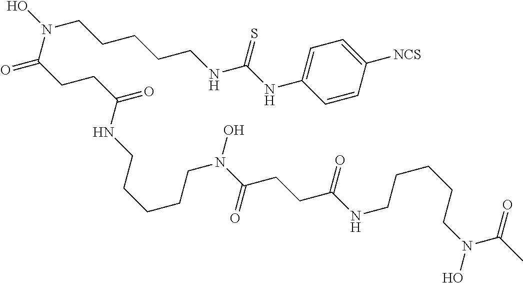 |
| DFO- cyclostar | 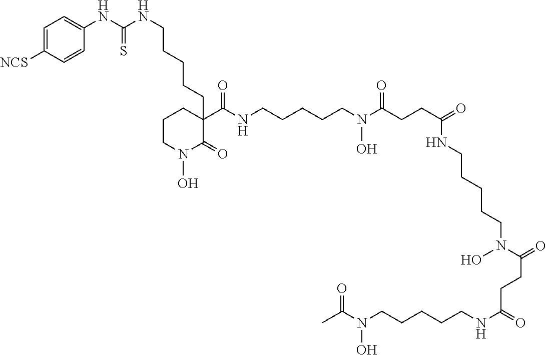 |
| DFO* | 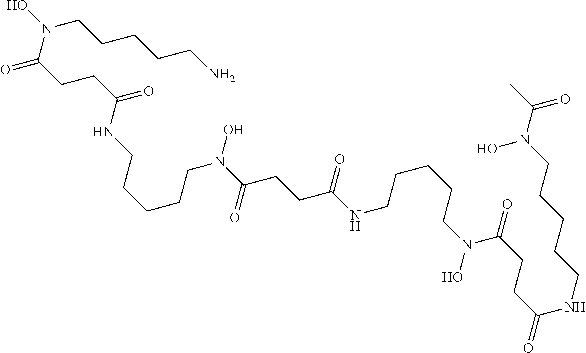 [SMILES: CC(=O)N(O)CCCCCNC(=O)CCC(=O)N(O)CCCCCNC(=O)CCC(=O)N(O)CCCCCNC(=O)CCC(=O)N(O)CCCCCN] |
| TAME | 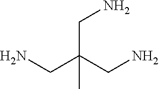 |
| TAME Hex | 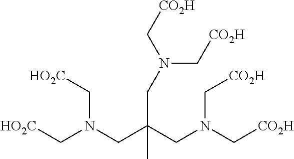 [SMILES: CC(CN(CC(=O)O)CC(=O)O)(CN(CC(=O)O)CC(=O)O)CN(CC(=O)O)CC(=O)O] |
| O- hydroxy- benzyl imino- diacetic acid | 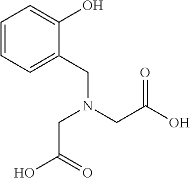 [SMILES: O=C(O)CN(CC(=O)O)Cc1ccccc1O] |
| TACN | 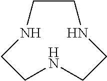 |
| Cyclen | 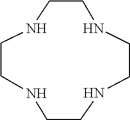 [SMILES: C1CNCCNCCNCCN1] |
| TETA | 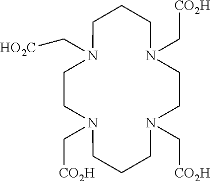 [SMILES: O=C(O)CN1CCCN(CC(=O)O)CCN(CC(=O)O)CCCN(CC(=O)O)CC1] |
| 2C-TETA | 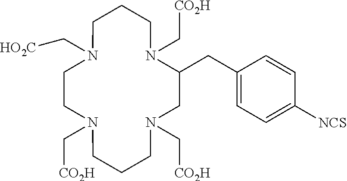 |
| 6C-TETA | 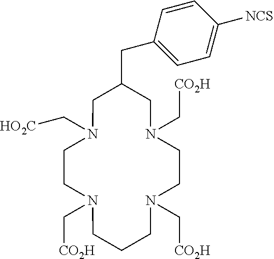 |
| PEPA | 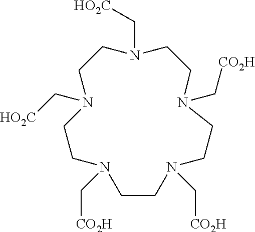 [SMILES: O=C(O)CN1CCN(CC(=O)O)CCN(CC(=O)O)CCN(CC(=O)O)CCN(CC(=O)O)CC1] |
| BF-PEPA | 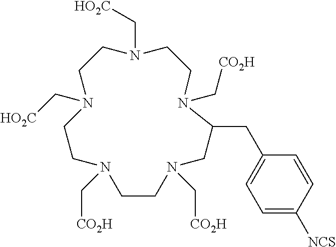 |
| HEHA | 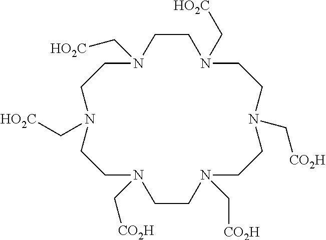 [SMILES: O=C(O)CN1CCN(CC(=O)O)CCN(CC(=O)O)CCN(CC(=O)O)CCN(CC(=O)O)CCN(CC(=O)O)CC1] |
| BF-HEHA | 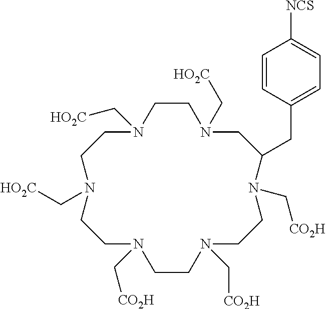 |
| TCMC | 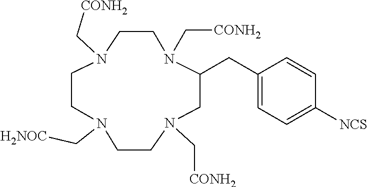 |
| Macropa | 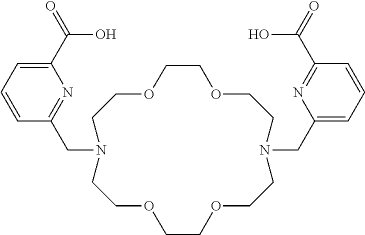 [SMILES: O=C(O)c1cccc(CN2CCOCCOCCN(Cc3cccc(C(=O)O)n3)CCOCCOCC2)n1] |
| Macropa- NCS | 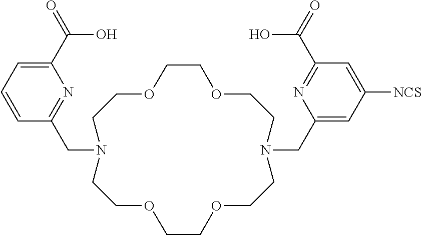 |
| Macrodipa | |
| Crown | |
| HOPO | 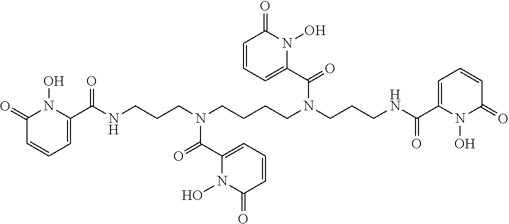 [SMILES: O=C(NCCCN(CCCCN(CCCNC(=O)c1cccc(=O)n1O)C(=O)c1cccc(=O)n1O)C(=O)c1cccc(=O)n1O)c1cccc(=O)n1O] |

Non-limiting examples of chelating agents include 1,4,7,10-tetraazacyclododecane-1,4,7,10-tetraacetic acid (DOTA); DOTA derivative: DO3A; diethylenetriamine-N,N,N′,N″,N″-pentaacetic acid (DTPA); DTPA derivatives: 2-(p-SCN-Bz)-6-methyl-DTPA, CHX-A″-DTPA, and the cyclic anhydride of DTPA (CA-DTPA); 1,4,7-triazacyclononane-1,4-7-triacetic acid (NOTA); NOTA derivatives (e.g., BCNOTA, p-NCS-Bz-NOTA, BCNOT); 6-hydrazinonicotinamide (HYNIC); ethylenediamine tetraacetic acid (EDTA); N,N′-ethylene-di-L-cysteine; N,N′-bis(2,2-dimethyl-2-mercaptoethyl)ethylenediamine-N,N′-diacetic acid (6SS); 1-(4-carboxymethoxybenzyl)-N—N′-bis [(2-mercapto-2,2-dimethyl)ethyl]-1,2-ethylenediamine-N,N′-diacetic acid (B6SS); Deferoxamine (DFO); 1,1,1-tris(aminomethyl)ethane (TAME); tris(aminomethyl)ethane-N,N,N′,N′,N″,N″-hexaacetic acid (TAME Hex); O-hydroxybenzyl iminodiacetic acid; 1,4,7-triazacyclononane (TACN); 1,4,7,10-tretraazacyclododecane (cyclen); 1,4,7-triazacyclononane-1-succinic acid-4,7-diacetic acid (NODASA); 1-(1-carboxy-3-carboxypropyl)-4,7-bis-(carboxymethyl)-1,4,7-triazacyclononane (NODAGA); 1,4,7-tris(2-mercaptoethyl)-1,4,7-triazacylclonane (triazacyclononane-TM); 1,4,7-triazacyclononane-N,N′,N″-tris(methylenephosphonic)acid (NOTP); 1,4,8,11-tetraazacyclotetradecane-N,N′,N″,N″-tetraacetic acid (TETA); 1,4,7,10,13-pentaazacyclopentadecane-N,N′,N″,N″,N″-pentaacetic acid (PEPA), 1,4,7,10,13,16-hexaazacyclohexadecane-N,N′,N″,N″,N″,N′″″-hexaacetic acid (HEHA); 1,4,7,10-tetrakis (carbamoylmethyl)-1,4,7,10-tetraazacyclododecane (TCMC); and derivatives or analogs thereof.

In an embodiment, the chelator is independently selected from the group consisting of ethylenediamine tetraacetic acid (EDTA), diethylenetriamine pentaacetic acid (DTPA), 1,4,7,10-tetra-azacylcododecane-N,N′,N”, N″-tetraacetic acid (DOTA), 6-((16-((6-Carboxypyridin-2-yl)methyl)-1,4,10,13-tetraoxa-7,16-diazacyclooctadecan-7-yl)methyl)-4-isothiocyanatopicolinic acid (Macropa), Macrodipa, 2,2′,2″,2′-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, a-(2-carboxyethyl) (DOTAGA), 1,4,7-Triazacyclononane-N,N′,N″-triacetic acid (NOTA), 1,4,7,10-tetraazacyclododecane-N,N′,N″,N″-tetraacetic acid (TETA), 1,4,7,10,13-pentaazacyclopentadecane-N,N′,N″,N″,N″″-pentaacetic acid (PEPA), and 1,4,7,10,13,16-hexaazacyclohexadecane-N,N′,N″,N″,N″,N″″-hexaacetic acid (HEHA). In another embodiment, the chelator is 5,11,16,22-Tetraazahexacosanediamide (DFO*) or N,N′-1,4-Butanediylbis[N-[3-[[(1,6-dihydro-1-hydroxy-6-oxo-2-pyridinyl)carbonyl]amino]propyl]-1,6-dihydro-1-hydroxy-6-oxo-2-pyridinecarboxamide] (HOPO).

### Linkers

Targeting construct linkers may include optional linkers connecting chelating agents and targeting moieties. Targeting construct linkers may link one or more chelating agents and one or more targeting moieties. Linkers may include one or more of an ester bond, disulfide, amide, acylhydrazone, ether, carbamate, carbonate, sulfonamide, alkyl, aryl, heteroaryl, thioether, and urea.

In some embodiments, linkers include cleavable linkers. In some embodiments, linkers include non-cleavable linkers. In some embodiments, optional linkers include amino acids.

As used herein, the term “linker” refers to a chemical moiety that joins a chelator to a peptide of the present disclosure. Any suitable linker known to those skilled in the art in view of the present disclosure can be used herein.

Linkers can act as electrophiles and bond to a nucleophilic portion of a chelator. Alternatively, linkers can act as nucleophiles and bond to a electrophilic portion of a chelator. It is understood that a linker may be attached to a chelator via the carbon backbone of the chelator allowing all “binding arms” of the chelator molecule to interact with the metal. Alternatively, one of the arms may be attached to the linker.

In some embodiments, the chelator is bound, via an amine group of the cyclic peptide or optional linker, to a carbonyl of the chelator, then an amide bond is formed between the chelator and the cyclic peptide or optional linker.

For example, when a chelator is DOTA and linker is PEG, then the resulting structure can be

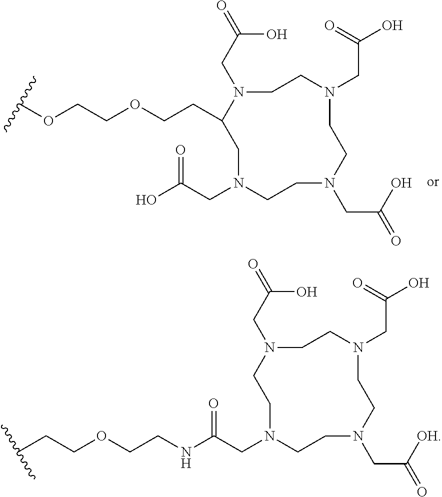

In yet another example, when a chelator is DOTA and the amino acid is Lys, then the resulting side chain of the amino acid can be

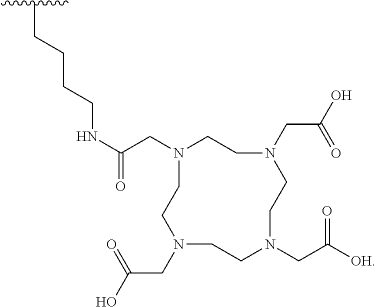 [SMILES: *CCCCNC(=O)CN1CCN(CC(=O)O)CCN(CC(=O)O)CCN(CC(=O)O)CC1]

### Cargos

Targeting constructs may include a variety of cargo. In some embodiments, cargo association with targeting constructs is facilitated by chelating agents. Cargo may include radioactive agents. Radioactive agent cargo associated with targeting constructs via chelating agents may include radionuclides. Chelating agents used for targeting construct association with such cargo may include metal chelating groups.

A variety of radionuclides have emission properties, including α, β, γ, and Auger emissions. These may be used with targeting constructs used for therapeutic and/or diagnostic purposes. For example, the targeting constructs may include Y-90, Y-86, I-131, Re-186, Re-188, Y-90, Bi-212, At-211, Zr-89, Sr-89, Ho-166, Sm-153, Cu-67, Cu-64, Lu-177, Ac-225, Pb-203, Bi-213, Th-227, Pb-212, Ra-223, P-32, Sc-47, Br-77, Rh-105, Pd-103, Ag-111, Pr-142, Pm-149, Gd-159, Ir-194 and/or Pt-199 radionuclides.

In some embodiments, targeting constructs used in imaging applications may include radionuclides cargo useful as imaging probes. Non-limiting examples of such radionuclides include, but are not limited to, I-124, I-131, In-111, Re-186, Re-188, Y-90, Bi-212, At-211, Sr-89, Ho-166, Sm-153, Cu-60, Cu-67, Cu-64, Lu-177, Ac-225, Bi-213, Th-227, Pb-212, Ra-223, P-32, Sc-47, Br-76, Br-77, Rh-105, Pd-103, Ag-111, Pr-142, Pm-149, Gd-159, In-111, Ir-194, Pt-199, Tc-99m, Co-57, Ga-66, Ga-67, Ga-68, Kr-81m, Rb-82, Sr-92, TI-201, Y-86, Zr-89, C-11, N-13, O-15 and F-18.

In some embodiments, targeting construct cargo includes any of the radionuclides listed in Table 4 including radionuclide parents and daughters thereof.

TABLE 4
Radionuclides

| | |
|---|---|
| C-11 | Rh-105 |
| N-13 | Ag-111 |
| O-15 | In-111 |
| F-18 | I-124 |
| P-32 | I-131 |
| Sc-47 | Pr-142 |
| Co-57 | Pm-149 |
| Cu-60 | Sm-153 |
| Cu-67 | Gd-159 |
| Cu-64 | Ho-166 |
| Ga-66 | Lu-177 |
| Ga-67 | Re-186 |
| Ga-68 | Re-188 |
| Br-76 | Ir-194 |
| Br-77 | Pt-199 |
| Kr-81m | Tl-201 |
| Rb-82 | Pb-203 |
| Y-86 | At-211 |
| Zr-89 | Pb-212 |
| Sr-89 | Bi-212 |
| Y-86 | Bi-213 |
| Y-90 | Ra-223 |
| Sr-92 | Ac-225 |
| Tc-99m | Th-227 |
| Pd-103 | Lu-175 |
| Ac-227 | In-115 |

In some embodiments, the radionuclide is a therapeutically active radionuclide. Suitable therapeutically active radionuclides include, but are not limited to, ³²P, ⁶⁷Cu, ¹⁸⁶Re, ¹⁸⁸Re, ⁸⁹Sr, ⁹⁰Y, ¹⁴³Ce, ¹⁷⁷Lu, ¹⁶¹Tb, ¹⁶⁶Ho, ¹⁶⁹Er, ¹⁸³Ta, ¹⁵³Sm, ²¹³Bi, ¹³¹I, ¹⁴⁹Tb, ⁴⁷Sc, ²²⁵Ac, ²¹²Pb, ²¹¹At, ²²³Ra, ²²⁷Th, and ²²⁶Th. In some embodiments, the radionuclide is a therapeutically active radionuclide selected from ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, and ²²⁷Th. In particular embodiments, the radionuclide is a therapeutically active radionuclide selected from ⁹⁰Y, ¹⁷⁷Lu, ¹³¹I, ²²⁵Ac, ²¹¹At, and ²²⁷Th. In a particular embodiment, the therapeutically active radionuclide is ¹⁷⁷Lu. In a particular embodiment, the therapeutically active radionuclide is ²²⁵ Ac.

Alternatively, in some embodiments, the radionuclide is a diagnostically active radionuclide. Suitable diagnostically active radionuclides include, but are not limited to, ¹¹¹ In, ^{99m}Tc, ^{94m}Tc, ⁶⁷Ga, ⁶⁸Ga, ²⁰³Pb, ⁶⁴Cu, ⁸⁶Y, ⁸⁹Zr, ⁵¹Mn, ⁵²Mn, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, and ²⁰¹TI. In some embodiments, the radionuclide is a diagnostically active radionuclide selected from ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Tb, ¹⁵⁵Tb, ⁴⁴Sc, and ⁴³Sc. In particular embodiments, the radionuclide is a diagnostically active radionuclide selected from ¹¹¹In, ^{99m}Tc, ⁶⁸Ga, ⁶⁴Cu, ⁸⁹Zr, ¹²³I, ¹²⁴I, and ¹⁸F. In a particular embodiment, the diagnostically active radionuclide is ¹¹¹In. In a particular embodiment, the diagnostically active radionuclide is ⁶⁸Ga. In another particular embodiment, the diagnostically active radionuclide is ¹⁸F. In another particular embodiment, the diagnostically active radionuclide is ⁶⁴Cu.

In some embodiments, the radionuclide is selected from the group consisting of ¹¹¹ In, ^{99m}Tc, ^{94m}Tc, ⁶⁶Ga, ⁶⁷Ga, ⁶⁸Ga, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh, ¹⁶⁵Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹²³I, ¹²⁴I, ¹²⁵I, ¹³¹I, ⁷⁵Br, ⁷⁶Br, ⁷⁷Br, ⁸⁰Br, ⁸²Br, ¹⁸F, ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac. In some embodiments, the radionuclide is ¹¹¹In, ^{99m}Tc, ⁶⁷Ga, ⁶⁸Ga, ²⁰³Pb, ⁶⁴Cu, ⁸⁶Y, ⁸⁹Zr, ¹²³I, ¹²⁴I, ¹²⁵I, ¹⁸F, ⁷⁶Br, ⁷⁷Br, ¹⁵²Tb, ¹⁵⁵Tb, ⁴⁴Sc, ⁴³Sc, ⁶⁷Cu, ¹⁸⁸Re, ⁹⁰Y, ¹⁷⁷Lu, ²¹³Bi, ¹³¹I, ⁴⁷Sc, ²²⁵Ac, ²¹²Pb, ²¹¹At, or ²²⁷Th. In some embodiments, the radionuclide is ⁶⁶Ga, ⁶⁷Ga, ⁶⁸Ga, ⁶⁴Cu, ¹⁷⁷Lu, or ²²⁵Ac. In some embodiments, the radionuclide is ¹¹¹In, ^{99m}Tc, ⁶⁸Ga, ⁶⁴Cu, ⁸⁹Zr, ¹²³I, ¹²⁴I, ¹⁸F, ⁹⁰Y, ¹⁷⁷Lu, ¹³¹I, ²²⁵Ac, ²¹¹At, or ²²⁷Th. In certain embodiments, the radionuclide is ¹⁷⁷Lu. In certain embodiments, the radionuclide is ²²⁵Ac. In certain embodiments, the radionuclide is ⁶⁸Ga. In certain embodiments, the radionuclide is ¹¹¹ In. In certain embodiments, the radionuclide is ¹⁸F.

In some embodiments, the radionuclide is ¹⁷⁷Lu, ¹⁶¹Tb, ⁹⁰Y, ⁶⁷Cu, ¹³¹I, ²²⁵Ac, ²¹²Pb, ²¹¹At, or ²²⁷Th.

In some embodiments, the radionuclide is a radiohalogen, e.g., ¹⁸F, ⁷⁵Br, ⁷⁶Br, ⁷⁷Br, ⁸⁰Br, ^{80m}Br, ⁸²Br, ¹²³I, ¹²⁴I, ¹²⁵I, ¹³¹I, or ²¹¹At. When the radionuclide is a radiohalogen, the term radiohalogen includes complexes that make the radiohalogen suitable for covalent attachment to the linker or the cyclic peptide or for chelation or complex formation with the chelator. Such complexes contemplated under the term radiohalogen include Si-¹⁸F, B-¹⁸F, and Al-¹⁸F.

In some embodiments, the radiohalogen is connected directly to the cyclic peptide or the linker. For example, ¹³¹I and ¹⁸F (or any other radiohalogen) can be substituted at any position of the linker or the cyclic peptide suitable for substitution with a halo group. In some embodiments, the radiohalogen is ¹⁸F. In some embodiments, when a radiohalogen is connected directly to the cyclic peptide or the linker, the chelator is absent.

In some embodiments, targeting constructs of the present disclosure can be radiolabeled with a radionuclide at any site of peptide that targets B7-H3. For example, in some embodiments the peptide that targets B7-H3 is conjugated directly to a radionuclide. In one embodiment, the radionuclide is covalently attached to the peptide that targets B7-H3. In another embodiment, the radionuclide can rely on ionic interactions, thereby forming a peptide radionuclide salt.

In some embodiments, the peptide that targets B7-H3 can be conjugated to a chelator. In one embodiment, the peptide that targets B7-H3 can be radiolabeled e.g., via chelation of the radionuclide to the chelator. Chelation of a radionuclide to a chelator may be depicted using solid single bonds, dashed single bonds, or a combination thereof. For example, the chelation of a radionuclide to DOTA can be depicted below with solid single bonds or dashed single bonds. In some embodiments, the charge may also be indicated. For example, when a radionuclide is chelated to a chelating agent, each of the groups chelating the radionuclide may have a negative charge and the radionuclide being chelated may have an opposing positive charge. Such bonds and charges may be depicted herein as follows in the case of ⁶⁸Ga:

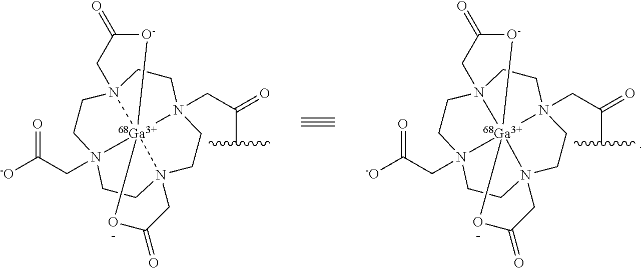

In the case of ²²⁵Ac, such bonds and charges may be illustrated (non-limiting) as follows:

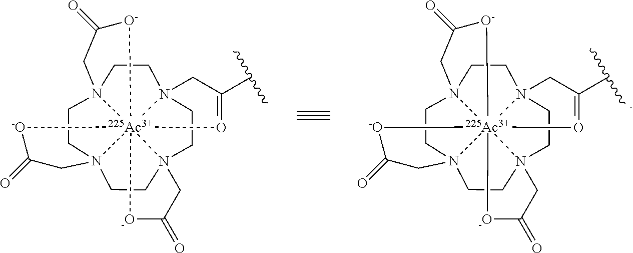

In another embodiments, the cyclic peptide, or a pharmaceutically acceptable salt or solvate thereof, is radiolabeled with F-18, Ga-68, In-111, Lu-177, or Ac-225.

In yet another embodiment, the cyclic peptide is selected from compounds 485, 494, 496, and 526.

In still another embodiment, compound 485, or a pharmaceutically acceptable salt or solvate thereof, is radiolabeled with F-18, Ga-68, In-111, Lu-177, or Ac-225.

In still another embodiment, compound 494, or a pharmaceutically acceptable salt or solvate thereof, is radiolabeled with F-18, Ga-68, In-111, Lu-177, or Ac-225.

In still another embodiment, compound 496, or a pharmaceutically acceptable salt or solvate thereof, is radiolabeled with F-18, Ga-68, In-111, Lu-177, or Ac-225.

In still another embodiment, compound 526, or a pharmaceutically acceptable salt or solvate thereof, is radiolabeled with F-18, Ga-68, In-111, Lu-177, or Ac-225.

In yet another embodiment, the cyclic peptide is selected from compounds 485a, 494a, 496a, and 526a.

In still another embodiment, compound 485a, or a pharmaceutically acceptable salt or solvate thereof, is radiolabeled with F-18, Ga-68, In-111, Lu-177, or Ac-225.

In still another embodiment, compound 494a, or a pharmaceutically acceptable salt or solvate thereof, is radiolabeled with F-18, Ga-68, In-111, Lu-177, or Ac-225.

In still another embodiment, compound 496a, or a pharmaceutically acceptable salt or solvate thereof, is radiolabeled with F-18, Ga-68, In-111, Lu-177, or Ac-225.

In still another embodiment, compound 526a, or a pharmaceutically acceptable salt or solvate thereof, is radiolabeled with F-18, Ga-68, In-111, Lu-177, or Ac-225.

Non-limiting examples of cyclic peptides radiolabeled with radionuclides are depicted in Table D.

TABLE D
Radiolabeled Cyclic Peptides*

| | Cpd |
|---|---|
| Structures | No. |
| 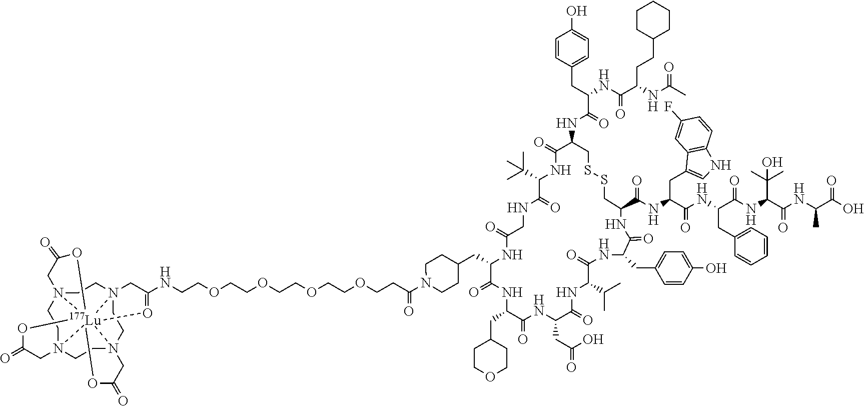 | [¹⁷⁷Lu] Lu- labeled 485 |
| 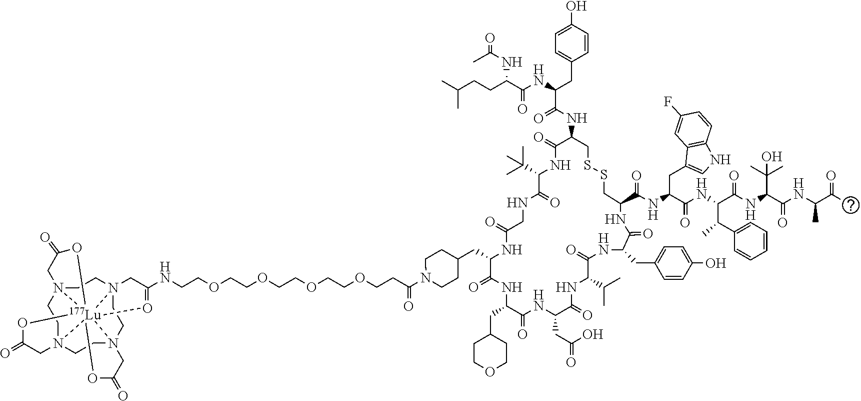 | [¹⁷⁷Lu] Lu- labeled 494 |
| 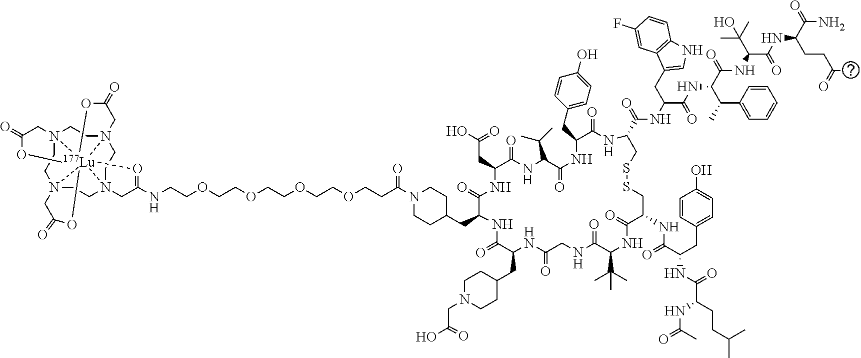 | [¹⁷⁷Lu] Lu- labeled 496 |
| 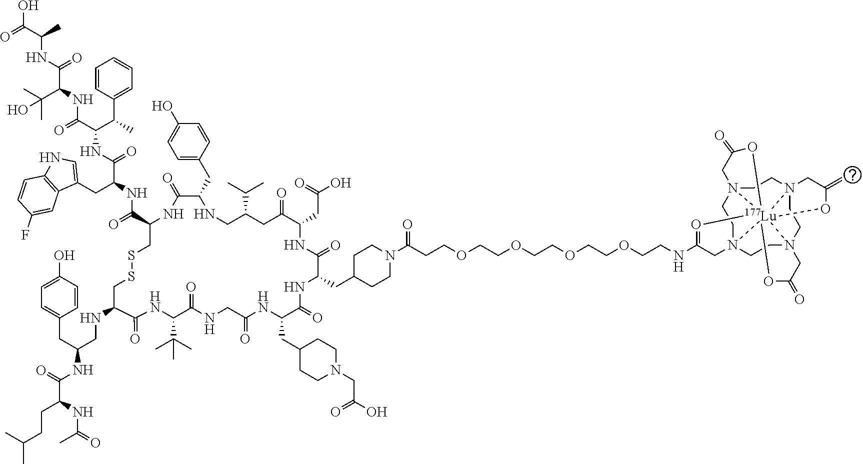 | [177Lu] Lu- labeled 526 |
| 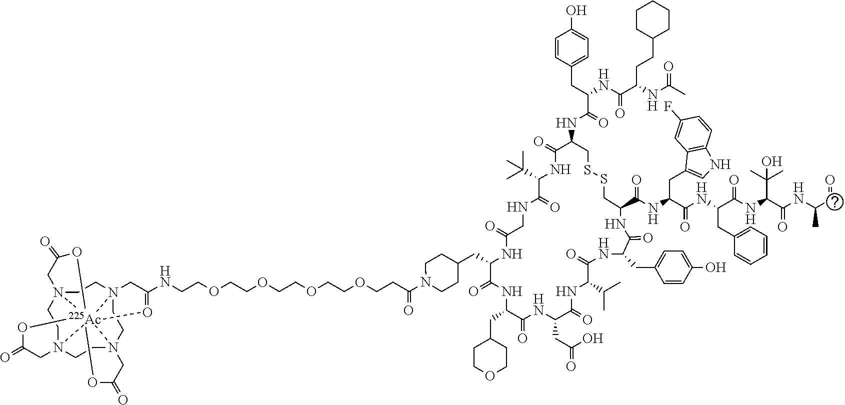 | [²²⁵Ac] Ac- labeled 485 |
| 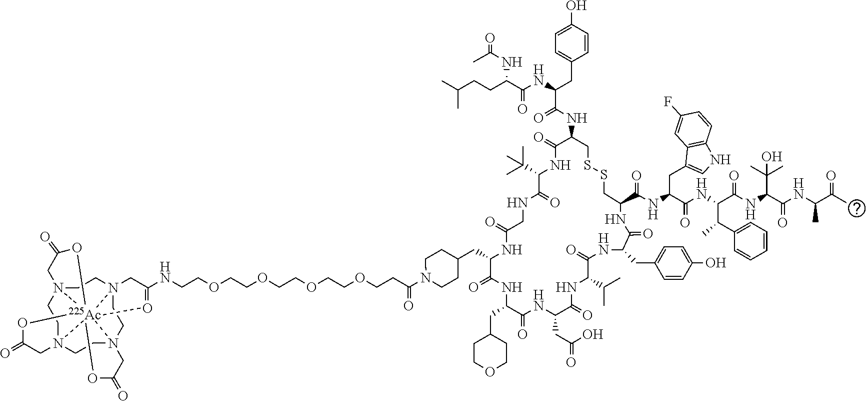 | [²²⁵Ac] Ac- labeled 494 |
| 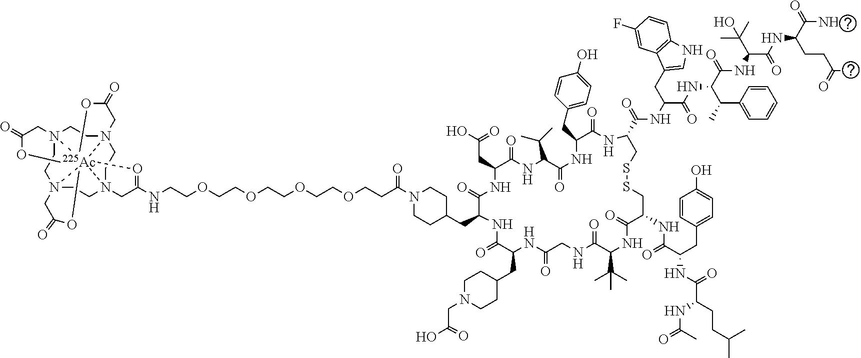 | [²²⁵Ac] Ac- labeled 496 |
| 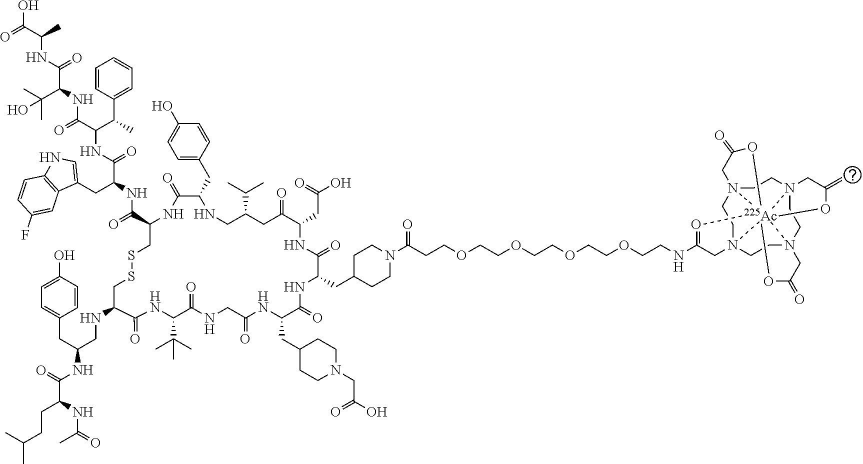 | [²²⁵Ac] Ac- labeled 526 |

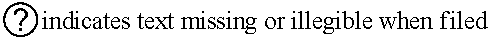

* The drawings above represent one way in which the [²²⁵Ac]Ac-DOTA complexes (²²⁵Ac radiolabeled cyclic peptides comprising DOTA) or [¹⁷⁷Lu]Lu-DOTA
complexes (¹⁷⁷Lu radiolabeled cyclic peptides comprising DOTA) could be illustrated.
The same compound could be drawn, e.g., with a different number of coordination bonds, represented by either dashed or solid lines.

The radionuclide may be a therapeutic radionuclide, diagnostic radionuclide, or both. Suitable radionuclides include, but are not limited to, auger-electron emitting radionuclides, β-emitting (beta-plus or beta-minus-emitting) radionuclides, and α-emitting (alpha-emitting) radionuclides. The selection of the type of radionuclide may depend on the use of the peptide that targets B7-H3. As will be appreciated by the skilled artisan, several factors may be considered when selecting a radionuclide for use in a peptide that targets B7-H3, such as, for example, the half-life, the linear energy transfer, the imaging capabilities, and the emission range in tissue. For example, β-emitting radionuclides typically have a longer emission range in tissue (e.g., 1-5 micrometer) and emit photons in an energy range that is easily imaged, and as such, they may be selected for use in a B7-H3-targeting compound being used for therapeutic, diagnostic, or theragnostic purposes. On the other hand, a-emitting radionuclides have a shorter emission range in tissue (e.g., 50-100 micrometer) and a high potency due to the amount of energy deposited per path length traveled (i.e., linear energy transfer), which is approximately 400 times greater than that of electrons (beta-minus particles) or positrons (beta-plus particles). Thus, a-emitting radionuclides may be selected for therapeutic uses in which high potency of the radionuclide is desired.

Accordingly, in some embodiments, the radionuclide is an a-emitting radionuclide. In other embodiments, the radionuclide is a β-emitting radionuclide. In yet other embodiments, the radionuclide is an auger-electron emitting radionuclide.

### Targeting Constructs

In some embodiments, the present disclosure provides constructs capable of localizing to and/or associating with targets. Such constructs that include any combination of a targeting moiety and a cargo are referred to herein as “targeting constructs.” Provided herein the targeting constructs can be directed to B7-H3.

As used herein, the term “targeting moiety” refers to a component of a targeting construct or combination of components involved in targeting construct localization to or association with a target. Cargo components of targeting constructs may include any one of a variety of compounds, including, but not limited to, chemical compounds, biomolecules, metals, polymeric molecules, therapeutic agents, cytotoxic agents, and radioactive agents. In a particular embodiment, the targeting construct comprises a targeting moiety that is a cyclic peptide that targets B7-H3, which is attached, via an optional linker, to a chelating agent for association of a radionuclide.

Targeting constructs of the present disclosure may include chelating agents. As used herein, the term “chelating agent” or “chelator” refers to any compound capable of forming two or more bonds with metal atoms. Chelating agents may facilitate targeting construct association with cargo that includes metal atoms. In a particular embodiment, the targeting construct comprises a chelating agent for association of a radionuclide.

The terms “chelated to” and “complexed with” as used herein is meant to indicate that two independent constituents are joined together such as by one or more non-covalent bonds, e.g., coordination bonds.

The term “radiolabeled” or “labeled” as used herein means that a non-radioactive compound is labeled with a radionuclide. Radiolabeling can be achieved, e.g., via chelation or complexation of a chelator with an appropriate radionuclide. Radiolabeling can also refer to chemically substituting one group on a compound for a radionuclide, e.g., by forming a covalent bond, such as, e.g., in the case of ¹⁸F.

Targeting construct components may be associated via one or more linkers. For example, targeting moieties may be associated with chelating agents or cargo via linkers. In some embodiments, linkers include chelating agents (e.g., where targeting construct cargo includes metal atoms).

In some embodiments, targeting constructs of the present disclosure include targeting moieties attached, optionally by a linker, to a cargo or a chelating agent for association of cargo. Targeting constructs may include a single targeting moiety and a single chelating agent, i.e., having the structure TM-L-CA, where “TM” is a targeting moiety, “L” is an optional linker, and “CA” is a chelating agent. Alternatively, targeting constructs may include a single targeting moiety and more than one chelating agent, e.g., a construct having the structure TM-L-(CA) n, wherein n is an integer representing the number of chelating agents. In some embodiments, n is an integer between 1 and 50, such as between 2 and 20, or between 1 and 5. Targeting constructs may have a structure of CA-L-TM-L-CA, wherein each L and each CA may be the same or different.

Targeting constructs optionally associated with radioactive cargo may be referred to herein according to corresponding analogs, i.e., the “radioactive analog” or the “non-radioactive analog” of a given targeting construct.

In some embodiments, targeting constructs may include detectable labels. Detectable labels may be used to detect antibody binding. Examples of detectable labels include, but are not limited to, radionuclides, fluorophores, chromophores, chemiluminescent compounds, enzymes, enzyme co-factors, dyes, metal ions, ligands, biotin, avidin, streptavidin, haptens, quantum dots, or any other detectable labels known in the art or described herein.

### Formulations

Also, provided herein is a formulation comprising a radiolabeled compound and a stabilizing solution. In an embodiment, the radiolabeled compound comprises a compound of Formula (I), a chelator, and optionally a linker.

In an embodiment, the stabilizing solution comprises one or more components selected from the group consisting of: ethanol, L-methionine, selenomethionine, histidine, melatonin, a polysorbate, ammonium acetate, ascorbic acid or a pharmaceutically acceptable salt thereof, acetic acid or a pharmaceutically acceptable salt thereof, benzyl alcohol, p-aminobenzoic acid or a pharmaceutically acceptable salt thereof, cysteamine, 5-amino-2-hydroxybenzoic acid or a pharmaceutically acceptable salt thereof, nicotinic acid or a pharmaceutically acceptable salt thereof, nicotinamide, cysteine, monothioglycerol, sodium bisulfite, sodium metabisulfite, gentisic acid, and inositol.

In another embodiment, the formulation comprises one or more components selected from the group consisting of: ethanol, L-methionine, a polysorbate, ascorbic acid or a pharmaceutically acceptable salt thereof, and acetic acid or a pharmaceutically acceptable salt thereof.

In yet another embodiment, the formulation comprises ascorbic acid or a pharmaceutically acceptable salt thereof, and a polysorbate. In an embodiment, the mass ratio of ascorbic acid or a pharmaceutically acceptable salt thereof to polysorbate in the formulation is from 50:1 to 150:1, optionally from 80:1 to 120:1, optionally about 100:1. In still another embodiment, the ascorbic acid or a pharmaceutically acceptable salt thereof is present in an amount of ≤100 mg/mL, optionally≤80 mg/mL, optionally from 30-70 mg/mL, optionally from 40-60 mg/mL, optionally about 50 mg/mL. In an embodiment, the polysorbate is present in the formulation in an amount of ≤0.1% (w/V) (corresponding to ≤1 mg/ml), optionally 0.03-0.07% (w/V) (corresponding to 0.3-0.7 mg/mL), optionally 0.04-0.06% (w/V) (corresponding to 0.4-0.6 mg/mL), optionally about 0.05% (w/V) (corresponding to 0.5 mg/mL). In another embodiment, the polysorbate is polyoxyethylene (20) sorbitan monolaurate (Tween 20), optionally wherein the polyoxyethylene (20) sorbitan monolaurate is present in the formulation in an amount of about 0.1 mg/ml to about 1 mg/mL.

In yet another embodiment, the formulation further comprises L-methionine, optionally wherein the L-methionine is present in the formulation at a concentration of about 10 mg/mL to about 30 mg/mL.

In still another embodiment, the formulation further comprises acetic acid or a pharmaceutically acceptable salt thereof, optionally wherein the acetic acid or pharmaceutically acceptable salt thereof is present in the formulation at a concentration of about 0.01 M to about 0.15 M. In an embodiment, the acetic acid or a pharmaceutically acceptable salt thereof is ammonium acetate. In another embodiment, the formulation further comprises ethanol, optionally wherein the ethanol is present in the formulation at about 1% v/v to about 15% v/v, optionally wherein the ethanol is present at about 5% v/v to about 10% v/V.

In yet another embodiment, the formulation comprises ethanol, L-methionine, a polyoxyethylene sorbitan monooleate, ascorbic acid or a pharmaceutically acceptable salt thereof, and acetic acid or a pharmaceutically acceptable salt thereof. In still another embodiment, the formulation comprises ethanol, ammonium acetate, sodium ascorbate, L-methionine, and polyoxyethylene (20) sorbitan monolaurate.

In an embodiment, the pH of the stabilized solution is about 6 and/or the pH of the formulation is about 6.

In another embodiment, the radiolabeled compound of the formulation comprises a radionuclide selected from ¹¹¹In, ^{99m}Tc, ^{94m}Tc, ⁶⁶Ga, ⁶⁷Ga, ⁶⁸Ga, ¹³⁴Ce, ⁵²Fe, ¹⁶⁹Er, ⁷²As, ⁹⁷Ru, ²⁰³Pb, ⁶¹Cu, ⁶²Cu, ⁶⁴Cu, ⁶⁷Cu, ⁸⁹Sr, ¹⁸⁶Re, ¹⁸⁸Re, ⁸⁶Y, ⁹⁰Y, ⁸⁹Zr, ⁵¹Cr, ⁵²Mn, ⁵¹Mn, ¹⁷⁷Lu, ¹⁶⁹Yb, ¹⁷⁵Yb, ¹⁰⁵Rh, ¹⁶⁶Dy, ¹⁶⁶Dy, ¹⁶⁶Ho, ¹⁵³Sm, ¹⁴⁹Pm, ¹⁵¹Pm, ¹⁷²Tm, ¹²¹Sn, ^{117m}Sn, ²¹²Bi, ²¹³Bi, ¹⁴²Pr, ¹⁴³Pr, ¹⁹⁸Au, ¹⁹⁹Au, ¹⁸F (e.g., [¹⁸F]AlF), ¹⁴⁹Tb, ¹⁵²Tb, ¹⁵⁵Tb, ¹⁶¹Tb, ⁴³Sc, ⁴⁴Sc, ⁴⁷Sc, ²¹²Pb, ²¹¹At, ²²³Ra, ²²⁷Th, ²²⁶Th, ⁸²Rb, ³²P, ⁷⁶As, ⁸⁹Zr, ¹¹¹Ag, ¹⁶⁵Er, ²²⁵Ac, and ²²⁷Ac. In still another embodiment, the radiolabeled compound comprises ¹¹¹In (i.e., the radionuclide is ¹¹¹In). In an embodiment, the radiolabeled compound comprises ¹⁷⁷Lu (i.e., the radionuclide is ¹⁷⁷Lu). In another embodiment, the radiolabeled compound comprises ²²⁵Ac (i.e., the radionuclide is ²²⁵Ac).

In some embodiments, the radiolabeled compound has an affinity for a cell surface protein. In another embodiment, the affinity in units K_{D} of ≤1.0 nM. In other embodiments, the affinity is characterized as: 1.0 nM<K_{D}≤10 nM, 10 nM<K_{D}≤100 nM, or 100 nM<K_{D}≤300 nM.

In an embodiment, the formulation has a total activity in the range of 0.01-1000 mCi. In some embodiments, the radionuclide is ¹⁷⁷Lu and the total activity of the formulation is in the range of 10-1000 mCi, for example, 10-100 mCi, 100-200 mCi, 200-300 mCi, 300-400 mCi, 400-500 mCi, 500-600 mCi, 600-700 mCi, 700-800 mCi, 800-900 mCi, or 900-1000 mCi. In some embodiments, the radionuclide is ²²⁵Ac and the total activity of the formulation is in the range of 0.01-5 mCi, for example, 0.1-5 mCi, 1-5 mCi, 2-5 mCi, 3-5 mCi, 4-5 mCi, 0.01-4 mCi. 0.1-4 mCi, 1-4 mCi, 2-4 mCi, 3-4 mCi, 0.01-3 mCi. 0.1-3 mCi, 1-3 mCi, 2-3 mCi, 0.01-2 mCi. 0.1-2 mCi, 1-2 mCi, 2-3, 0.01-1 mCi, or 0.1-1 mCi.

In another embodiment, the formulation has a volume of about 1-100 mL. In an embodiment, the formulation has a volume of about 1-10 mL. In another embodiment, the formulation has a volume of about 11-20 mL. In yet another embodiment, the formulation has a volume of about 15 mL. In an embodiment, the formulation has a volume of about 21-30 mL. In still another embodiment, the formulation has a volume of about 31-40 mL. In an embodiment, the formulation has a volume of about 41-50 mL. In another embodiment, the formulation has a volume of about 41 mL.

In yet another embodiment, the formulation has a volume of about 51-60 mL. In still another embodiment, the formulation has a volume of about 61-70 mL. In an embodiment, the formulation has a volume of about 71-80 mL. In another embodiment, the formulation has a volume of about 81-90 mL. In yet another embodiment, the formulation has a volume of about 91-100 mL.

In an embodiment, the formulation comprises sodium ascorbate, ascorbic acid, polyoxyethylene (20) sorbitan monolaurate, and acetate.

In yet another embodiment, the formulation comprises sodium ascorbate, ascorbic acid, polyoxyethylene (20) sorbitan monolaurate, and ethanol.

In another embodiment, the formulation comprises sodium ascorbate, L-methionine, polyoxyethylene (20) sorbitan monolaurate, ethanol, and acetate.

In still another embodiment, the formulation comprises sodium ascorbate in a concentration of about 40 mg/mL to about 60 mg/mL, L-methionine in a concentration of about 10 mg/mL to about 30 mg/mL, polyoxyethylene (20) sorbitan monolaurate in an amount of from about 0.01% w/v to about 0.1% w/v, ethanol in an amount of from about 1% v/v to about 10% v/v, and acetate in a concentration of about 0.01 M to about 0.1 M.

In some embodiments, compositions are administered to humans, human patients or subjects. For the purposes of the present disclosure, the phrase “active ingredient” generally refers to the constructs as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g., non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The constructs of the present disclosure can be formulated using one or more excipients to: (1) increase stability; (2) permit the sustained or delayed release; (3) alter the biodistribution; (4) alter the release profile of the compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present disclosure may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the disclosure may include one or more excipients, each in an amount that together increases the stability of the compounds.

### Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and Kolliphor® (SOLUTOL®)), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g., sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Administration

The constructs of the present disclosure may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier.

The formulations described herein contain an effective amount of constructs in a pharmaceutical carrier appropriate for administration to an individual in need thereof. The formulations may be administered parenterally (e.g., by injection or infusion). The formulations or variations thereof may be administered in any manner including enterally, topically (e.g., to the eye), or via pulmonary administration. In some embodiments the formulations are administered topically.

### Dosing

The present disclosure provides methods comprising administering constructs as described herein to a subject in need thereof. Constructs as described herein may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like.

Compositions in accordance with the disclosure are typically formulated in dosage unit form for ease of administration and uniformity of dosage. It will be understood, however, that the total daily usage of the compositions of the present disclosure may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present disclosure may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, from about 25 mg/kg to about 50 mg/kg, from about 50 mg/kg to about 100 mg/kg, from about 100 mg/kg to about 125 mg/kg, from about 125 mg/kg to about 150 mg/kg, from about 150 mg/to about 175 mg/kg, from about 175 mg/kg to about 200 mg/kg, from about 200 mg/kg to about 250 mg/kg of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

The concentration of the constructs may be between about 0.01 mg/mL to about 50 mg/mL, about 0.1 mg/mL to about 25 mg/mL, about 0.5 mg/mL to about 10 mg/mL, or about 1 mg/mL to about 5 mg/mL in the pharmaceutical composition.

As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose.

In some embodiments, the total dose (over the course of a treatment regimen) of the B7-H3 targeting construct comprising a β-emitter such as, e.g., 177Lu, is from about 1 GBq to about 200 GBq. In some embodiments, the B7-H3 targeting construct comprising a β-emitter is administered in a total dose to deliver from 40 to 100 GBq of radiation. In some embodiments, the B7-H3 targeting construct comprising the β-emitter is administered in a single dose (once within a 24-hour period) to deliver from about 1 to about 20 GBq of radiation. In some embodiments, B7-H3 targeting construct comprising the β-emitter is administered in a single dose (once within a 24-hour period) to deliver from about 3 to about 15 GBq of radiation. In some embodiments, B7-H3 targeting construct comprising the β-emitter is administered in a single dose (once within a 24-hour period) to deliver from about 5 to about 10 GBq of radiation.

In some embodiments, the total dose (over the course of a treatment regimen) of the B7-H3 targeting construct comprising an α-emitter, e.g., ²²⁵Ac, is from about 1 MBq to about 100 MBq, e.g., about 4 MBq to about 80 MBq, e.g., about 5 MBq to about 77 MBq, e.g., about 5 MBq, about 6MBq, about 8 MBq, about 10 MBq, about 13 MBq, and about 76 MBq. In some embodiments, the B7-H3 targeting construct comprising an α-emitter is administered in a total dose of from about 20 to about 80 MBq of radiation. In some embodiments, the B7-H3 targeting construct comprising an α-emitter is administered in a single dose (once within a 24-hour period) to deliver from about 1 to about 40 MBq of radiation. In some embodiments, the B7-H3 targeting construct comprising an α-emitter is administered in a single dose (once within a 24-hour period) to deliver from about 5 to about 40 MBq of radiation. In some embodiments, B7-H3 targeting construct comprising an a-emitter is administered in a single dose (once within a 24-hour period) to deliver from about 5 to about 25 MBq of radiation.

In some embodiments, the total dose (over the course of a treatment regimen) of the B7-H3 targeting construct comprising an α-emitter, e.g., ²²⁵Ac, is administered to the subject once about every 4 to 10 weeks. In another embodiment, the construct is administered to the subject once about every 6 to 8 weeks. In still another embodiment, the construct is administered to the subject once about every 6 weeks. In yet another embodiment, the construct is administered to the subject once about every 6 weeks for 4 to 6 cycles.

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, and subcutaneous).

### II. Methods

In some embodiments, the present disclosure provides methods related to preparing, using, and evaluating compounds (e.g., targeting constructs) and compositions disclosed herein.

### Therapeutic Applications

In some embodiments, methods of the present disclosure include methods of treating therapeutic indications using compounds and/or compositions disclosed herein. As used herein, the term “therapeutic indication” refers to any symptom, condition, disorder, or disease that may be alleviated, stabilized, improved, cured, or otherwise addressed by some form of treatment or other therapeutic intervention. In some embodiments, methods of the present disclosure include treating therapeutic indications by targeting constructs disclosed herein.

By “lower” or “reduce” in the context of a disease marker or symptom is meant a significant decrease in such a level, often statistically significant. The decrease may be, for example, at least 10%, at least 20%, at least 30%, at least 40% or more, and is preferably down to a level accepted as within the range of normal for an individual without such a disorder.

By “increase” or “raise” in the context of a disease marker or symptom is meant a significant rise in such level, often statistically significant. The increase may be, for example, at least 10%, at least 20%, at least 30%, at least 40% or more, and is preferably up to a level accepted as within the range of normal for an individual without such disorder.

Efficacy of treatment or amelioration of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. In connection with the administration of a compound or composition described herein, “effective against” a disease or disorder indicates that administration in a clinically appropriate manner results in a beneficial effect for at least a fraction of patients, such as an improvement of symptoms, a cure, a reduction in disease load, extension of life, improvement in quality of life, or other effect generally recognized as positive by medical doctors familiar with treating the particular type of disease or disorder.

A treatment or preventive effect is evident when there is a significant improvement, often statistically significant, in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more may be indicative of effective treatment. Efficacy for a given compound or composition may also be judged using an experimental animal model for the given disease as known in the art. When using an experimental animal model, efficacy of treatment is evidenced when a statistically significant modulation in a marker or symptom is observed.

In some embodiments, methods of the present disclosure include administering targeting constructs described herein to treat hyperproliferative diseases, metabolic diseases, infectious diseases, and/or cancer. Targeting construct formulations may be administered by multiple routes, including, but not limited to, injection, oral administration, or topical administration. In some embodiments, administration is to a mucosal surface (lung, nasal, oral, buccal, sublingual, vaginally, rectally) or to the eye (intraocularly or transocularly).

### Cancer

In an aspect, provided herein is a method of targeting B7-H3 in a subject in need thereof, comprising administering to the individual a therapeutically effective amount of a compound disclosed herein.

In another aspect, provided herein is a method of treating cancer in a subject in need thereof, comprising administering to the individual a therapeutically effective amount of a compound disclosed herein.

In an embodiment, the cancer is a B7-H3-mediated cancer. In another embodiment, the cancer is lung cancer, urothelial cancer, melanoma, squamous cell carcinoma, endometrial cancer, breast cancer, acute myeloid leukemia (AML), gastric cancer, colorectal cancer, prostate cancer, glioma, ovarian cancer, liver cancer, cervical cancer, esophageal cancer, and head and neck cancer. In yet another embodiment, the cancer is small cell lung cancer, urothelial cancer, melanoma, or squamous cell carcinoma. In still another embodiment, the cancer is urothelial cancer, melanoma, lung cancer, squamous cell carcinoma, breast cancer, esophageal cancer, prostate cancer, liver cancer, endometrial cancer, sarcoma, bladder cancer, salivary gland cancer, renal cell carcinoma, gastric cancer, or pancreatic cancer. In an embodiment, the cancer is non-small cell lung cancer (NSCLC), small cell lung cancer (SCLC), triple-negative breast cancer (TNBC), Luminal A breast cancer, Luminal B breast cancer, HER2+ breast cancer, head and neck squamous cell carcinoma (HNSCC), or osteosarcoma.

In yet another embodiment, the cancer is selected from the group consisting of acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor.

In another embodiment, the cancer is selected from the group consisting of primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningeal cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharyngeal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomyosarcoma, multiple myeloma, ovarian cancer, glioblastoma, gastrointestinal stromal tumor, mantle cell lymphoma, and refractory malignancy.

In an embodiment, the cancer is selected from the group consisting of breast, ovary, cervix, prostate, testis, genitourinary tract, esophagus, larynx, glioblastoma, neuroblastoma, stomach, skin, keratoacanthoma, lung, epidermoid carcinoma, large cell carcinoma, small cell carcinoma, lung adenocarcinoma, bone, colon, colorectal, adenoma, pancreas, adenocarcinoma, thyroid, follicular carcinoma, undifferentiated carcinoma, papillary carcinoma, seminoma, melanoma, sarcoma, bladder carcinoma, liver carcinoma and biliary passages, kidney carcinoma, myeloid disorders, lymphoid disorders, Hodgkin's, hairy cells, buccal cavity and pharynx (oral), lip, tongue, mouth, pharynx, small intestine, colon, rectum, large intestine, rectum, brain and central nervous system, chronic myeloid leukemia (CML), and leukemia.

In another embodiment, the cancer is selected from the group consisting of myeloma, lymphoma, or a cancer selected from gastric, renal, head and neck, oropharyngeal, non-small cell lung cancer (NSCLC), endometrial, hepatocarcinoma, non-Hodgkin's lymphoma, and pulmonary.

In an embodiment, the cancer is selected from the group consisting of prostate cancer, colon cancer, lung cancer, squamous cell cancer of the head and neck, esophageal cancer, hepatocellular carcinoma, melanoma, sarcoma, gastric cancer, pancreatic cancer, ovarian cancer, breast cancer.

In an embodiment, the cancer is selected from the group consisting of tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotropic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer that may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in treating are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngioma, osteosarcoma, chondrosarcoma, myosarcoma, liposarcoma, fibrosarcoma, Ewing sarcoma, and plasmacytoma.

In another aspect, the disclosure provides a compound disclosed herein, or a pharmaceutically acceptable salt thereof, for use in the manufacture of a medicament for treating a disease in which B7-H3 plays a role.

One aspect of this disclosure provides compounds that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer.

In another aspect, provided herein is the use of one or more compounds of the disclosure in the manufacture of a medicament for the treatment of cancer, including without limitation the various types of cancer disclosed herein.

In some embodiments, therapeutic indications include cancer-related indications. The term “cancer” refers to a collection of diseases characterized by dysfunctional cell growth and division, in some cases spreading between bodily regions. As used herein, the term “cancer-related indication” refers to any disease, disorder, or condition pertaining to cancer, cancer treatment, or pre-cancerous conditions. Cancer-related indications include, but are not limited to, pathological conditions characterized by malignant neoplastic growths, tumors, and/or hematological malignancies. In some embodiments, methods of the present disclosure include treatment of cancer-related indications with targeting constructs of the present disclosure.

In various embodiments, methods for treating cancer are provided, wherein the method comprises administering a therapeutically-effective amount of the constructs, salt forms thereof, as described herein, to a subject having a cancer, suspected of having cancer, or having a predisposition to a cancer. According to the present disclosure, cancer embraces any disease or malady characterized by uncontrolled cell proliferation, e.g., hyperproliferation. Cancers may be characterized by tumors, e.g., solid tumors or any neoplasm.

In some embodiments, the subject may be otherwise free of indications for treatment with the constructs. In some embodiments, methods include use of cancer cells, including but not limited to mammalian cancer cells. In some instances, the mammalian cancer cells are human cancer cells.

In some embodiments, constructs according to the present disclosure inhibit cancer and/or tumor growth. They may also reduce one or more of cell proliferation, invasiveness, and metastasis, thereby making them useful for cancer treatment.

In some embodiments, the constructs of the present teachings may be used to prevent the growth of a tumor or cancer, and/or to prevent the metastasis of a tumor or cancer. In some embodiments, compositions of the present teachings may be used to shrink or destroy a cancer.

In some embodiments, the constructs provided herein are useful for inhibiting proliferation of a cancer cell. In some embodiments, the constructs provided herein are useful for inhibiting cellular proliferation, e.g., inhibiting the rate of cellular proliferation, preventing cellular proliferation, and/or inducing cell death. In general, the constructs as described herein can inhibit cellular proliferation of a cancer cell or both inhibiting proliferation and/or inducing cell death of a cancer cell. In some embodiments, cell proliferation is reduced by at least about 25%, about 50%, about 75%, or about 90% after treatment with constructs of the present disclosure compared with cells with no treatment. In some embodiments, cell cycle arrest marker phospho histone H3 (PH3 or PHH3) is increased by at least about 50%, about 75%, about 100%, about 200%, about 400% or about 600% after treatment with constructs of the present disclosure compared with cells with no treatment. In some embodiments, cell apoptosis marker cleaved caspase-3 (CC3) is increased by at least 50%, about 75%, about 100%, about 200%, about 400% or about 600% after treatment with constructs of the present disclosure compared with cells with no treatment.

Furthermore, in some embodiments, constructs of the present disclosure are effective for inhibiting tumor growth, whether measured as a net value of size (weight, surface area or volume) or as a rate over time, in multiple types of tumors.

In some embodiments the size of a tumor is reduced by about 60% or more after treatment with constructs of the present disclosure. In some embodiments, the size of a tumor is reduced by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 100%, by a measure of weight, and/or area and/or volume.

The cancers treatable by methods of the present teachings generally occur in mammals. Mammals include, for example, humans, non-human primates, dogs, cats, rats, mice, rabbits, ferrets, guinea pigs horses, pigs, sheep, goats, and cattle. In various embodiments, cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningeal cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharyngeal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomyosarcoma, multiple myeloma, ovarian cancer, glioblastoma, gastrointestinal stromal tumor, mantle cell lymphoma, and refractory malignancy.

In some embodiments, targeting constructs of the present disclosure are used to target cancer cells expressing B7-H3. In some embodiments, targeting constructs of the present disclosure are used to treat lung cancer, breast cancer, bladder cancer, colon cancer, urothelial cancer, melanoma, or squamous cell carcinoma.

### Theranostics

“Theranostics,” a term derived from a combination of therapeutics and diagnostics, is an emerging field of medicine where specific disease-targeting agents, e.g., radiopharmaceuticals, may be used to simultaneously or sequentially diagnose and treat medical conditions. Theranostics has become an important field of research and development in medical physics, where varying the isotope of the radionuclide present in a given disease-targeting agent, e.g., a radioligand therapy, can change the disease-targeting agent from an imaging probe (by, e.g., using β+ or γ emitting isotopes to facilitate positron emission tomography (PET) or single photon emission computed tomography (CT) imaging, respectively), to a therapy probe (by, e.g., using a or β-particle or Auger electron emitting isotopes to facilitate targeted radiotherapy).

Molecular imaging is a well-known and useful technique for in vivo diagnostics. It may be used in a wide variety of methods including the three-dimensional mapping of molecular processes, such as gene expression, blood flow, physiological changes (pH, etc.), immune responses and cell trafficking. It can be used to detect and diagnose disease, select optimal treatments, and to monitor the effects of treatments to obtain an early readout of efficacy.

A number of distinct technologies can in principle be used for molecular imaging, including PET, single photon emission tomography (SPET), optical magnetic resonance imaging (MRI), CT, and Cerenkov luminescence imaging (CLI). Combinations of these modalities are emerging to provide improved clinical applications, e.g., PET/CT and SPET/CT (“multi-modal imaging”).

Radionuclide imaging with PET and SPET has the advantage of extremely high sensitivity and small amounts of administered contrast agents (e.g., picomolar in vivo), which do not perturb the in vivo molecular processes. Moreover, the targeting principles for radionuclide imaging can be applied also in targeted delivery of radionuclide therapy. Typically, the isotope that is used as a radionuclide in molecular imaging or therapy is incorporated into a molecule to produce a radiotracer that is pharmaceutically acceptable to the subject.

As such, the constructs of the present disclosure can be used in radiotherapy as well as medical imaging for diagnostics. The constructs provided herein can also be used to simultaneously or sequentially diagnose and treat medical conditions

### Combination Therapies

In some embodiments, constructs of the present disclosure are combined with at least one additional active agent. The active agent may be any suitable drug. The constructs and the at least one additional active agent may be administered simultaneously, sequentially, or at any order. The constructs and the at least one additional active agent may be administered at different dosages, with different dosing frequencies, or via different routes, whichever is suitable.

In some embodiments, the additional active agents affect the biodistribution (i.e., tissue distribution) of the constructs of the current disclosure. For example, radioactive agents may accumulate in kidneys and may pose a potential radiotoxicity problem to kidneys and surrounding organs. The additional active agent may reduce renal accumulation or retention time. Preferably, kidney update of the constructs is reduced, while tumor uptake of the constructs is not affected. Kidney and surrounding organs are protected without reducing the efficacy of the constructs. In one non-limiting example, constructs of the current disclosure may be administered in combination with at least one amino acid or analog(s) thereof. The amino acid or analog(s) thereof may be positively charged basic amino acids such as lysine (L-lysine or D-lysine) or arginine, or a combination thereof.

The additional active agent may also be selected from any active agent described herein such as a drug for treating cancer. It may also be a cancer symptom relief drug. Non-limiting examples of symptom relief drugs include: octreotide or lanreotide; interferon, cypoheptadine or any other antihistamines. In some embodiments, constructs of the present disclosure do not have drug-drug interference with the additional active agent. The additional active agent may be administered concomitantly with constructs of the present disclosure.

In some embodiments, a non-radioactive analog of the construct the present disclosure may be combined with a radioactive analog of this construct. For example, the non-radioactive construct can be administered prior to the radioactive analog. In another example, a subject may receive a mixture of the non-radioactive construct and its radioactive analog. In yet another example, a subject may receive the non-radioactive construct treatment first, followed by a mixture of the non-radioactive construct and its radioactive analog.

In some embodiments, a construct of the present disclosure comprising one radiolabel may be combined with at least one other construct of the present disclosure comprising one or more different radiolabels. For example, constructs comprising an imaging radiolabel may be combined with constructs comprising a non-imaging radiolabel. In one embodiment, constructs associated with lutetium (Lu) may be combined with constructs associated with gallium (Ga).

The constructs as described herein or formulations containing the constructs as described herein can be used for the selective tissue delivery of a therapeutic, prophylactic, or diagnostic agent to an individual or patient in need thereof. For example, constructs of the present disclosure are used to deliver radioactive agents to selective tissues. These tissues may be tumor tissues. Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. Dosage unit form as used herein refers to physically discrete units suited as unitary dosages for the mammalian subjects to be treated; each unit containing a predetermined quantity of active compound calculated to produce the desired therapeutic effect.

### Diagnostic Applications

In some embodiments, the present disclosure provides diagnostic methods involving use of targeting moieties and or the targeting constructs. Such methods may include detecting B7-H3 using any of the targeting moieties and or the targeting constructs described herein. Such methods may include contacting subjects or subject samples with targeting moieties and or the targeting constructs described herein. The peptides and/or targeting constructs may bind to B7-H3. In a particular embodiment, the targeting construct comprises a targeting moiety that is a cyclic peptide that targets B7-H3. Targeting moieties and/or the targeting constructs used for detection methods may include a detectable label. Detection methods may include the use of detection reagents to detect bound antibodies or peptides. As used herein, the term “detection reagent” refers to any compound or substance used to visualize or otherwise observe an object (e.g., a bound antibody or detectable label) or event. Detection reagents may include secondary antibodies or other high affinity compounds (e.g., biotin or avidin) that bind to antibodies being detected or associated conjugates. Detection reagents may be or include substrates for detection of enzymatic detectable labels (e.g., associated with a primary or secondary antibody).

Diagnostic applications of the present disclosure may include detecting B7-H3 in subject samples that include cells. In some embodiment cell-associated B7-H3 may be detected. Cell-associated B7-H3 may be detected in subject samples by fluorescence-associated cell sorting (FACS) analysis. In some embodiments, B7-H3 may be detected in subject samples by immunohistochemistry. Such methods may include the use of colorimetric-based systems or immunofluorescence-based systems for B7-H3 detection. In a particular embodiment, the targeting construct comprises a targeting moiety that is a cyclic peptide that targets B7-H3.

In some embodiments, the present disclosure provides methods of stratifying subjects based on detection of B7-H3 in subjects or subject samples. Such methods may include detecting B7-H3 in subjects or subject samples according to any of the methods described herein (e.g., using peptides or targeting constructs comprising peptides and classifying subjects according to level of B7-H3 detected. In some embodiments, subjects may be classified according to the presence or absence of B7-H3 and/or level of B7-H3 in subjects or subject samples. Subjects may be further classified according to the presence or absence of specific B7-H3 extracellular subdomains and/or levels of specific B7-H3 extracellular subdomains in subjects or subject samples. Classifications used in subject stratification may include, but are not limited to, classifications by disease type, disease prognosis or severity, suitability for treatment, and type of treatment most likely to be successful or appropriate.

### Iii. Kits and Devices

The disclosure provides a variety of kits and devices for conveniently and/or effectively carrying out methods of the present disclosure. Typically, kits will comprise sufficient amounts and/or numbers of components to allow a user to perform multiple treatments of a subject(s) and/or to perform multiple experiments.

In one embodiment, the present disclosure provides kits for inhibiting cancer cell growth in vitro or in vivo, comprising a construct of the present disclosure or a combination of constructs of the present disclosure, optionally in combination with any other active agents.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of the constructs in the buffer solution over a period of time and/or under a variety of conditions.

The present disclosure provides for devices which may incorporate constructs of the present disclosure. These devices contain in a stable formulation available to be immediately delivered to a subject in need thereof, such as a human patient. In some embodiments, the subject has cancer.

Non-limiting examples of the devices include a pump, a catheter, a needle, a transdermal patch, a pressurized olfactory delivery device, iontophoresis devices, multi-layered microfluidic devices. The devices may be employed to deliver constructs of the present disclosure according to single, multi- or split-dosing regiments. The devices may be employed to deliver constructs of the present disclosure across biological tissue, intradermal, subcutaneously, or intramuscularly.

### IV. Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “alkylene,” employed alone or in combination with other terms, refers to a divalent alkyl linking group. An alkylene group formally corresponds to an alkane with two C—H bonds replaced by points of attachment of the alkylene group to the remainder of the compound. The term “Cₙ₋ₘ alkylene” refers to an alkylene group having n to m carbon atoms. Examples of alkylene groups include, but are not limited to, methylene, ethan-1,2-diyl, ethan-1,1-diyl, propan-1,3-diyl, propan-1,2-diyl, propan-1,1-diyl, butan-1,4-diyl, butan-1,3-diyl, butan-1,2-diyl, 2-methyl-propan-1,3-diyl and the like.

As used herein, the terms “associated with,” “conjugated,” “linked,” “attached,” and “tethered,” when used with respect to two or more entities, means that the entities are physically associated or connected with one another, either directly or via one or more moieties that serve as linking agents, to form a structure that is sufficiently stable so that the entities remain physically associated, e.g., under working conditions, e.g., under physiological conditions. An “association” need not be through covalent chemical bonding and may include other forms of association or bonding sufficiently stable such that the “associated” entities remain physically associated, e.g., ionic or hydrogen bonding or a hybridization-based connectivity.

As used herein, the term “optionally substituted” means that the referenced group may be unsubstituted (no substituents) or substituted, including with one or more additional groups individually and independently selected from groups described herein.

As used herein, the term “substituted” refers to substitution by independent replacement of one, two, or three or more of the hydrogen atoms with substituents described herein.

As used herein, the term “cancer” refers to a disease characterized by abnormal cell growth and division.

As used herein, the term “cancer cell” refers to a cell that grows and divides in an abnormal and uncontrolled manner.

As used herein, the term “compound,” refers to a distinct chemical entity. Constructs, targeting constructs, targeting moieties, cargo, chelators, or other construct components, together with any fragments or variants of the foregoing, may be referred to independently or collectively as compounds.

Compounds may exist in one or more isomeric or isotopic forms (including, but not limited to stereoisomers, geometric isomers, tautomers, and isotopes). Compounds may be provided or utilized in singular form or as a mixture of two or more forms (including, but not limited to racemic mixtures of stereoisomers). Some compounds may exist in different forms, which may exhibit different properties and/or activities (including, but not limited to biological activities). For example, compounds containing asymmetrically substituted carbon atoms may be isolated in optically active or racemic forms. As used herein, the below structure indicates the presence of a double bond wherein substituents can be configured as an E or Z isomer:

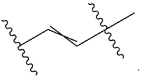 [SMILES: *C=C*]

The compounds described herein may be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of compounds of the present disclosure may be isolated as a mixture of isomers or as separated isomeric forms.

Tautomeric compound forms result from the swapping of a single bond with an adjacent double bond and concomitant migration of a proton. Tautomeric forms include prototropic tautomers which are isomeric protonation states having the same empirical formula and total charge. Examples prototropic tautomers include ketone-enol pairs, am-ide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds described herein may be provided in forms that include different isotopes of compound atoms. “Isotopes” refers to atoms having the same atomic number but different mass numbers resulting from a different number of neutrons in the nuclei. For example, isotopes of hydrogen include tritium and deuterium.

Compounds described herein may be provided as salts and may be prepared in combination with solvent or water molecules to form solvates and hydrates by routine methods.

As used herein, the term “hydrate” refers to the complex formed by the combining of a compound of Formula A, Formula I, or any formula disclosed herein, and water.

The term “solvate” refers to a complex formed by the combining of a compound of Formula A, Formula I, or any other formula as disclosed herein, and a solvent or a crystalline solid containing amounts of a solvent incorporated within the crystal structure. As used herein, the term “solvate” includes hydrates.

As used herein, the term “construct” refers to an artificially manipulated molecule. Some constructs may include nucleic acids and/or peptides, which may be products of recombinant technology and may be artificially synthesized or expressed from a recombinant nucleic acid sequence. Constructs may be combinations of nucleic acids, peptides, and/or other compounds.

As used herein, the term “cyclic” refers to the presence of a continuous loop. Cyclic molecules need not be circular, only joined to form an unbroken chain of subunits. Cyclic peptides may include a “cyclic loop,” formed when two amino acids are connected by a bridging moiety. The cyclic loop comprises the amino acids along the peptide present between the bridged amino acids. Cyclic loops may comprise 2, 3, 4, 5, 6, 7, 8, 9, 10 or more amino acids.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, an “epitope” refers to a surface or region on one or more entities that is capable of interacting with an antibody or other binding biomolecule. For example, a protein epitope may contain one or more amino acids and/or post-translational modifications (e.g., phosphorylated residues) which interact with an antibody. In some embodiments, an epitope may be a “conformational epitope,” which refers to an epitope involving a specific three-dimensional arrangement of the entity(ies) having or forming the epitope. For example, conformational epitopes of proteins may include combinations of amino acids and/or post-translational modifications from folded, non-linear stretches of amino acid chains.

As used herein, the term “equilibrium dissociation constant” or “K_{D}” refers to a value representing the tendency of two or more agents (e.g., two proteins) to reversibly separate. In some cases, K_{D} indicates a concentration of a primary agent at which half of the total levels of a secondary agent are associated with the primary agent.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a peptide or protein; and (4) post-translational modification of a peptide or protein.

As used herein, the term “half-life” or “t_{1/2}” refers to the time it takes for a given process or compound concentration to reach half of a final value. The “terminal half-life” or “terminal t_{1/2}” refers to the time needed for the plasma concentration of a factor to be reduced by half after the concentration of the factor has reached a pseudo-equilibrium.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “lactam bridge” refers to an amide bond that forms a bridge between chemical groups in a molecule. In some cases, lactam bridges are formed between amino acids in a peptide.

As used herein, a “linker” refers to any chemical structure that connects two or more entities or domains. Linkers may include one or more chemical bonds, atoms, groups of atoms, and/or chemical groups. Examples of chemical groups that can be included in linkers include, but are not limited to, alkyl, alkenyl, alkynyl, amido, amino, ether, thioether, ester, alkylene, heteroalkylene, aryl, or heterocyclyl chemical groups, each of which can be optionally substituted, as described herein. Linkers may include one or more of unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers. Linkers may include amino acids, peptides, peptides, and/or proteins.

Linkers may include carbon chains. Linker carbon chain lengths may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more atoms long. Linker carbon chains may contain heteroatoms (e.g., nitrogen, oxygen, sulfur, etc.).

Entities or domains joined by linkers may include, but are not limited to, atoms, chemical groups, nucleosides, nucleotides, nucleobases, sugars, nucleic acids, amino acids, peptides, peptides, proteins, protein complexes, cargo, therapeutic agents, and detectable labels. Linkers may be used for multiple purposes, including, but not limited to, forming multimers or conjugates.

Linkers may include cleavable elements, for example, disulfide (—S—S—) bonds or azo (—N═N—) bonds, which can be cleaved using reducing agents or photolysis. Selectively cleavable bonds may include amido bonds which may be cleaved for example by photolysis or by using tris(2-carboxyethyl)phosphine (TCEP) or other reducing agents. Selectively cleavable bonds may include ester bonds which may be cleaved, for example, by acidic or basic hydrolysis.

Linkers may include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers.

As used herein, the term “peptide backbone” consists of repeat units of an amino group, an a-carbon, and a carbonyl group (e.g., —NH₂—CH—C(O—)).

As used herein, the term “modulation” refers to up regulation (i.e., activation or stimulation) or down regulation (i.e., inhibition or suppression) of a response, or the two in combination or apart. Modulation is generally compared to a baseline or reference that can be internal or external to a treated entity.

As used herein, the term “patient” refers to a subject seeking treatment, in need of treatment, requiring treatment, receiving treatment, expecting treatment, or that are under the care of a trained (e.g., licensed) professional for a particular disease, disorder, or condition. Patients may include any organism. Patient treatments may include, but are not limited to, experimental, diagnostic, prophylactic, and/or therapeutic treatments. Typical patients include, but are not limited to, animals (e.g., mammals such as mice, rats, rabbits, non-human primates, and humans).

As used herein, the term “pharmaceutical composition” refers to a composition comprising at least one active ingredient in a form and amount that permits the active ingredient to be therapeutically effective.

The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

The term “pharmaceutically acceptable”, as used herein, refers to compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio (e.g., in accordance with the guidelines of government agencies or other regulatory bodies, for example, the U.S. Food and Drug Administration).

The phrase “pharmaceutically acceptable excipient,” as used herein, refers any ingredient other than active agents (e.g., as described herein) present in a pharmaceutical composition and having the properties of being substantially nontoxic and non-inflammatory in a patient.

As used herein, the term “pharmaceutically acceptable salt” refers to derivatives of the disclosed cyclic peptides wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “sample” or “biological sample” refers to a subset of its tissues, cells or component parts (e.g., body fluids, including but not limited to blood, serum, plasma, mucus, lymphatic fluid, synovial fluid, cerebrospinal fluid, saliva, amniotic fluid, and urine). Samples may further include a homogenate, lysate, or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, and organs. Samples may further refer to a medium, such as a nutrient broth or gel, which may contain cellular components or other biological materials, such as proteins (e.g., antibodies) or nucleic acid molecules.

As used herein, the term “subject” refers to any entity to which a particular process or activity relates to or is applied. Subjects may include any organism. Typical subjects include, but are not limited to, animals (e.g., mammals such as mice, rats, rabbits, non-human primates, and humans) and/or plants.

As used herein, the term “target” refers to an object or entity to be affected by an action or refers to activity associated with an agent that is directed to the object or entity (e.g., an agent that “targets” an object or entity). In some embodiments, targets refer to antigens, epitopes, or other structures to which antibodies or other compounds bind or that are selected and/or used in the design, development, or isolation of antigen-specific antibodies or other compounds. Targets may include molecular structures that include, but are not limited to, nucleic acids, peptides, proteins, haptens, receptors, carbohydrates, glycans, enzymes, lipids, cells, and fragments or complexes of any of the foregoing.

When used to refer to activity of an agent directed to objects or entities, the term “target” may be used to describe binding activity of agents (e.g., antibodies or related structures) with such objects or entities (e.g., antigens or epitopes). For example, an antibody that binds to a specific antigen may be said to “target” or be “directed to” the particular antigen. Similarly, a compound (e.g., a targeting construct) that exhibits activity (e.g., therapeutic or cytotoxic activity) toward a specific cell or tissue may be said to “target” the cell or tissue.

Targets may include cells (referred to herein as “target cells”). Target cells may be in vivo or in vitro. Target cells may include, for example, blood cells, lymph cells, cells lining the alimentary canal, such as the oral and pharyngeal mucosa, cells forming the villi of the small intestine, cells lining the large intestine, cells lining the respiratory system (nasal passages/lungs) of an animal, dermal/epidermal cells, cells of the vagina and rectum, cells of internal organs, cells of the placenta, and cells of the blood-brain barrier. In some embodiments, target cells may be cancer cells, including, but not limited to those found in leukemias or tumors (e.g., tumors of the brain, lung (small cell and non-small cell), ovary, prostate, breast, and colon, as well as other carcinomas and sarcomas). In still other embodiments, target cells may be part of a tissue. Tissues with target cells or other target structures are referred to herein as target tissues. Target tissues may include, but are not limited to, neuronal tissues, intestinal tissues, pancreatic tissues, liver tissues, kidney tissues, prostate tissues, ovary tissues, lung tissues, bone marrow tissues, and breast tissue tissues.

As used herein, the term “target site” refers to a precise region on or within a target that is acted on by a given effector. Target sites may be precise regions or epitopes recognized by antibodies or compounds. In some embodiments, target sites may reside exclusively on one or more monomers of polymeric structures (e.g., nucleic acids, peptides, polysaccharides, etc.). Target sites may be formed by junctions or regions of overlap between two or more monomers or compounds.

As used herein, the term “therapeutically effective amount” means an amount of an agent to be delivered that is sufficient, when administered to a subject suffering from or susceptible to a disease, disorder, and/or condition, to treat, improve symptoms of, diagnose, prevent, and/or delay the onset of the disease, disorder, and/or condition.

As used herein the terms “treat,” “treatment,” and the like, refer to any actions taken to offer relief from or alleviation of pathological processes. As it relates to any of the therapeutic indications recited herein, the terms “treat,” “treatment,” and the like mean to relieve or alleviate at least one symptom associated with such indications, or to slow or reverse the progression or anticipated progression of such indications.

As used herein, the term “contacting” refers to the bringing together of indicated moieties in an in vitro system or an in vivo system. For example, “contacting” a cell with a compound includes the administration of a compound of the present invention to an individual, subject, or patient, such as a human, as well as, for example, introducing a compound into a sample containing a purified preparation containing the cell.

As used herein, the term “cell” is meant to refer to a cell that is in vitro, ex vivo, or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, “specific molar activity” or “specific activity” refers to the measured radioactivity of a composition comprising a radiolabeled compound per moles of the compound (i.e., total moles of the radiolabeled compound and moles of unlabeled compound, if present). A composition comprising only radiolabeled compound will be characterized as having the theoretical maximum specific molar activity. A composition comprising both radiolabeled and unlabeled compound will be characterized has having a specific molar activity that is less than the theoretical maximum, and the specific molar activity decreases as the ratio of unlabeled compound to radiolabeled compound increases. Compositions that are enriched in radiolabeled compound (e.g., using the methods disclosed herein) are referred to as “high specific activity.” Non-enriched compositions are referred to as “regular specific activity” or “low specific activity.” High specific activity (HSA) compositions of radiolabeled compounds are desirable for treatment of diseases (e.g., cancer) characterized by low-expressing targets (e.g., DLL3). Such compositions as described herein are also advantageous in that they are purified to reduce or remove undesired byproducts and/or unreacted radioisotope and compound substances. However, HSA radiolabeled compounds undergo radioactive decay faster than radiolabeled compounds with low specific activity (LSA). Thus, stabilized radiolabeled compounds and the corresponding methods used to make them are critical for effective treatment.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “tumor” refers to a group of cells forming in solid tissue as a result of abnormal cell growth and division. Benign or “noncancerous” tumors remain isolated while malignant or “cancerous” tumors include cells capable of proliferating to 5 surrounding tissues.

As used herein, the term “tumor cell” refers to a cell associated with or derived from a tumor. Benign or “noncancerous” tumor cells remain associated with tumors while malignant or “cancerous” tumor cells are capable of proliferating to surrounding tissues.

As used herein, the following abbreviations are defined by the structures in Table C.

TABLE C

| Abbreviation | Structure |
|---|---|
| (2S,3S- betaMe-5FW) | 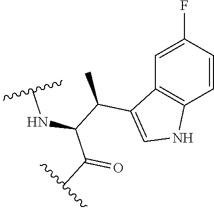 [SMILES: *N[C@H](C(*)=O)[C@@H](C)c1c[nH]c2ccc(F)cc12] |
| [(2S, 3R)beta- Me-Phe] | 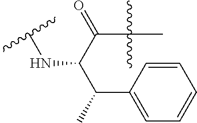 [SMILES: *N[C@H](C(*)=O)[C@@H](C)c1ccccc1] |
| [(2S, 3R)beta- OH-Phe] | 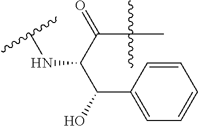 [SMILES: *N[C@H](C(*)=O)[C@@H](O)c1ccccc1] |
| [(2S, 3S)beta- Me-Phe] | 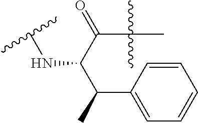 [SMILES: *N[C@H](C(*)=O)[C@H](C)c1ccccc1] |
| 1Nal | 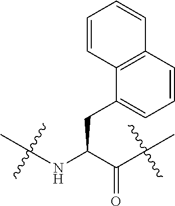 [SMILES: *N[C@@H](Cc1cccc2ccccc12)C(*)=O] |
| 2FPhe | 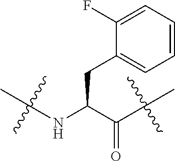 [SMILES: *N[C@@H](Cc1ccccc1F)C(*)=O] |
| 2MePhe (or 2MeF) | 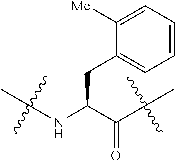 [SMILES: *N[C@@H](Cc1ccccc1C)C(*)=O] |
| 2MeW (or 2MeTrp) | 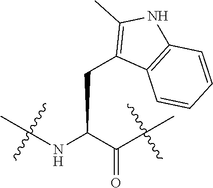 [SMILES: *N[C@@H](Cc1c(C)[nH]c2ccccc12)C(*)=O] |
| 2Nal | 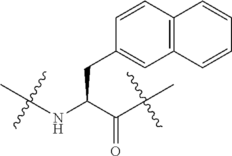 [SMILES: *N[C@@H](Cc1ccc2ccccc2c1)C(*)=O] |
| 2OHPhe | 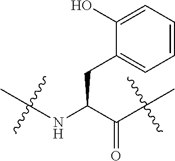 [SMILES: *N[C@@H](Cc1ccccc1O)C(*)=O] |
| 3aminomethylPhe | 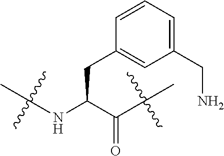 [SMILES: *N[C@@H](Cc1cccc(CN)c1)C(*)=O] |
| 3ClPhe | 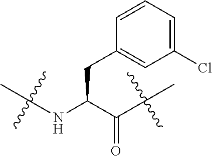 [SMILES: *N[C@@H](Cc1cccc(Cl)c1)C(*)=O] |
| 3ClY | 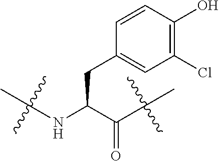 [SMILES: *N[C@@H](Cc1ccc(O)c(Cl)c1)C(*)=O] |
| 3CNPhe | 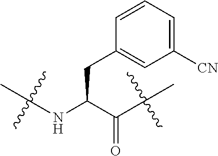 [SMILES: *N[C@@H](Cc1cccc(C#N)c1)C(*)=O] |
| 3FPhe | 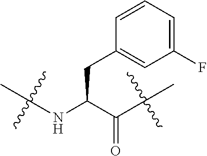 [SMILES: *N[C@@H](Cc1cccc(F)c1)C(*)=O] |
| 3FY (or 3FTyr) | 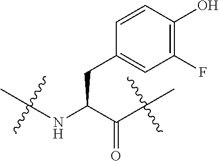 [SMILES: *N[C@@H](Cc1ccc(O)c(F)c1)C(*)=O] |
| 3OHPhe | 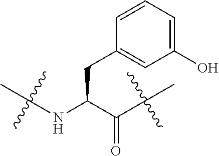 [SMILES: *N[C@@H](Cc1cccc(O)c1)C(*)=O] |
| 3PyA | 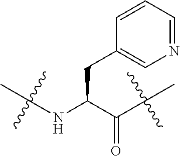 [SMILES: *N[C@@H](Cc1cccnc1)C(*)=O] |
| 4aminomethylPhe | 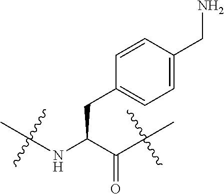 [SMILES: *N[C@@H](Cc1ccc(CN)cc1)C(*)=O] |
| 4-carbamoyl-Phe | 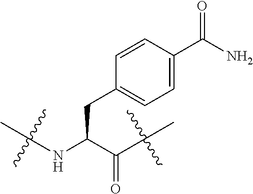 [SMILES: *N[C@@H](Cc1ccc(C(N)=O)cc1)C(*)=O] |
| 4ClW (or 4ClTrp) | 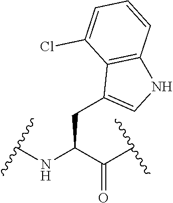 [SMILES: *N[C@@H](Cc1c[nH]c2cccc(Cl)c12)C(*)=O] |
| 4CNPhe (or 4CNF) | 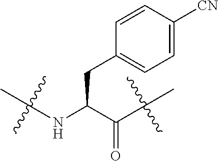 [SMILES: *N[C@@H](Cc1ccc(C#N)cc1)C(*)=O] |
| 4COOHPhe | 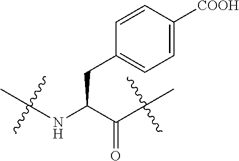 [SMILES: *N[C@@H](Cc1ccc(C(=O)O)cc1)C(*)=O] |
| 4FPhe | 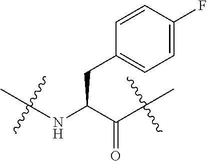 [SMILES: *N[C@@H](Cc1ccc(F)cc1)C(*)=O] |
| 4FW | 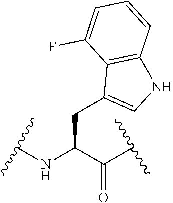 [SMILES: *N[C@@H](Cc1c[nH]c2cccc(F)c12)C(*)=O] |
| 4MeOW | 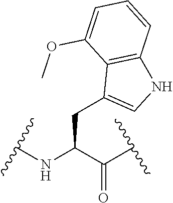 [SMILES: *N[C@@H](Cc1c[nH]c2cccc(OC)c12)C(*)=O] |
| 4MeW | 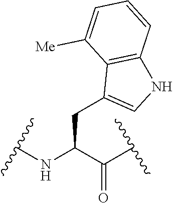 [SMILES: *N[C@@H](Cc1c[nH]c2cccc(C)c12)C(*)=O] |
| 4OHCha | 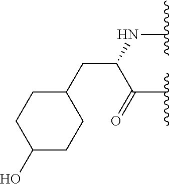 [SMILES: *N[C@@H](CC1CCC(O)CC1)C(*)=O] |
| 4OHPro | 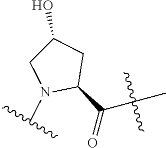 [SMILES: *C(=O)[C@@H]1C[C@@H](O)CN1*] |
| 4PyA | 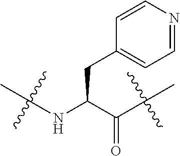 [SMILES: *N[C@@H](Cc1ccncc1)C(*)=O] |
| 5ClW (or 5ClTrp) | 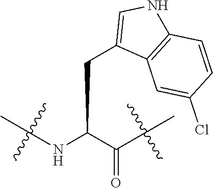 [SMILES: *N[C@@H](Cc1c[nH]c2ccc(Cl)cc12)C(*)=O] |
| 5FW | 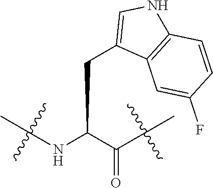 [SMILES: *N[C@@H](Cc1c[nH]c2ccc(F)cc12)C(*)=O] |
| 5MeOW | 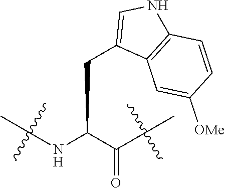 [SMILES: *N[C@@H](Cc1c[nH]c2ccc(OC)cc12)C(*)=O] |
| 5MeW | 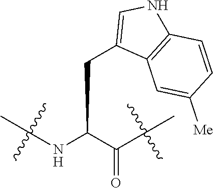 [SMILES: *N[C@@H](Cc1c[nH]c2ccc(C)cc12)C(*)=O] |
| 7AzaW (or 7NW) | 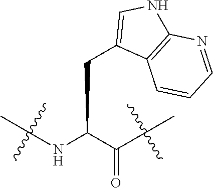 [SMILES: *N[C@@H](Cc1c[nH]c2ncccc12)C(*)=O] |
| AB-PEG8 | 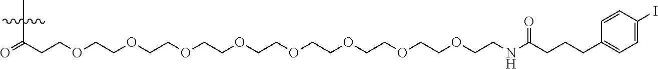 [SMILES: *C(=O)CCOCCOCCOCCOCCOCCOCCOCCOCCNC(=O)CCCc1ccc(I)cc1] |
| Abu | 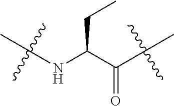 [SMILES: *N[C@@H](CC)C(*)=O] |
| AEF(DOTA) | 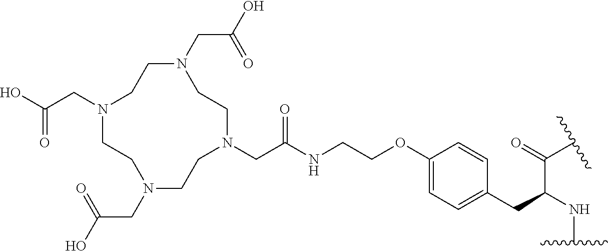 [SMILES: *N[C@@H](Cc1ccc(OCCNC(=O)CN2CCN(CC(=O)O)CCN(CC(=O)O)CCN(CC(=O)O)CC2)cc1)C(*)=O] |
| Aib | 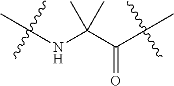 [SMILES: *NC(C)(C)C(*)=O] |
| A (or Ala) | 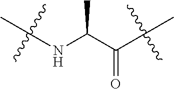 [SMILES: *N[C@@H](C)C(*)=O] |
| alloIle | 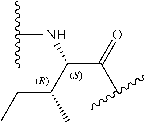 |
| alloThr | 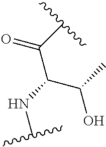 [SMILES: *N[C@H](C(*)=O)[C@H](C)O] |
| aMeLeu | 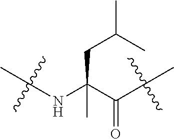 [SMILES: *N[C@](C)(CC(C)C)C(*)=O] |
| aMeLys (or aMeK) | 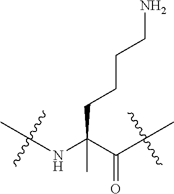 [SMILES: *N[C@](C)(CCCCN)C(*)=O] |
| aMeNle | 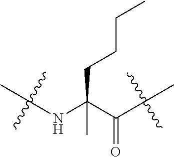 [SMILES: *N[C@](C)(CCCC)C(*)=O] |
| aMePhe (or aMeF) | 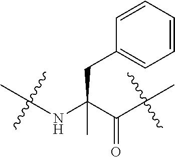 [SMILES: *N[C@](C)(Cc1ccccc1)C(*)=O] |
| aMePro (or alpha-Me-Pro) | 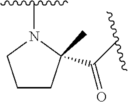 [SMILES: *C(=O)[C@]1(C)CCCN1*] |
| aMeSer (or aMeS) | 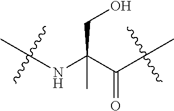 [SMILES: *N[C@](C)(CO)C(*)=O] |
| aMeTyr (or aMeY) | 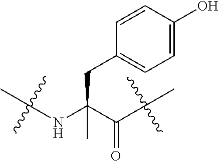 [SMILES: *N[C@](C)(Cc1ccc(O)cc1)C(*)=O] |
| aMeVal (or aMeV) | 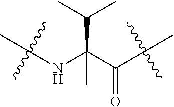 [SMILES: *N[C@@](C)(C(*)=O)C(C)C] |
| aMeW (or aMeTrp) | 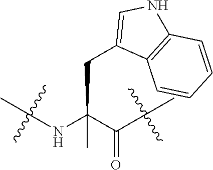 [SMILES: *N[C@](C)(Cc1c[nH]c2ccccc12)C(*)=O] |
| Aph(Cbm) | 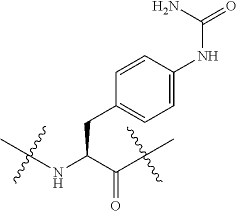 [SMILES: *N[C@@H](Cc1ccc(NC(N)=O)cc1)C(*)=O] |
| R (or Arg) | 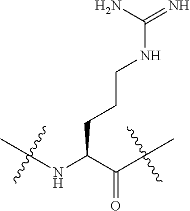 [SMILES: *N[C@@H](CCCNC(=N)N)C(*)=O] |
| Aze | 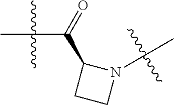 [SMILES: *C(=O)[C@@H]1CCN1*] |
| betaAla | 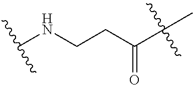 [SMILES: *NCCC(*)=O] |
| betaMeIle | 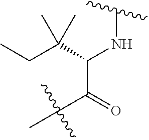 [SMILES: *N[C@H](C(*)=O)C(C)(C)CC] |
| C (or Cys) | 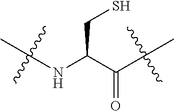 [SMILES: *N[C@@H](CS)C(*)=O] |
| C4diacid | 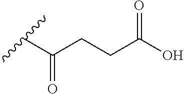 [SMILES: *C(=O)CCC(=O)O] |
| Cba | 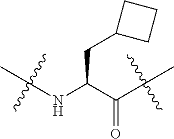 [SMILES: *N[C@@H](CC1CCC1)C(*)=O] |
| Cha | 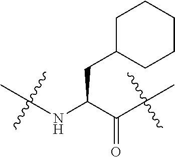 [SMILES: *N[C@@H](CC1CCCCC1)C(*)=O] |
| Chg | 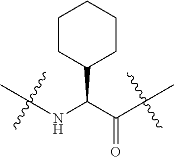 [SMILES: *N[C@H](C(*)=O)C1CCCCC1] |
| cis4NH2-Pro | 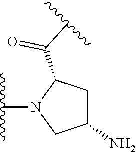 [SMILES: *C(=O)[C@@H]1C[C@H](N)CN1*] |
| trans4NH2-Pro | 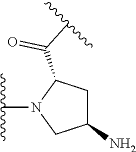 [SMILES: *C(=O)[C@@H]1C[C@@H](N)CN1*] |
| cis4FPro (or cis4Fluoro-Pro) | 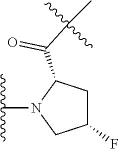 [SMILES: *C(=O)[C@@H]1C[C@H](F)CN1*] |
| cisHyp (or cis4OH-Pro) | 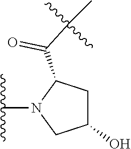 [SMILES: *C(=O)[C@@H]1C[C@H](O)CN1*] |
| Cit | 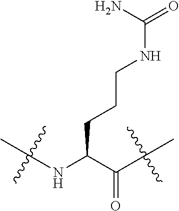 [SMILES: *N[C@@H](CCCNC(N)=O)C(*)=O] |
| Cpg | 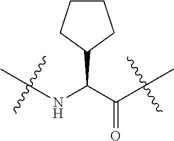 [SMILES: *N[C@H](C(*)=O)C1CCCC1] |
| CyanoButric | 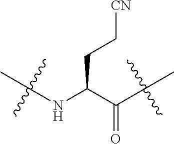 [SMILES: *N[C@@H](CCC#N)C(*)=O] |
| CysAcid | 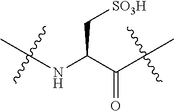 [SMILES: *N[C@@H](CS(=O)(=O)O)C(*)=O] |
| D (or Asp) | 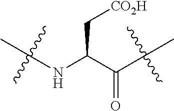 [SMILES: *N[C@@H](CC(=O)O)C(*)=O] |
| dA (or dAla) | 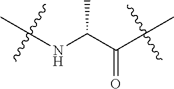 [SMILES: *N[C@H](C)C(*)=O] |
| Dab | 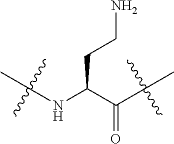 [SMILES: *N[C@@H](CCN)C(*)=O] |
| Dap | 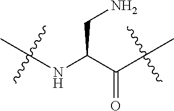 [SMILES: *N[C@@H](CN)C(*)=O] |
| dCys (or dC) | 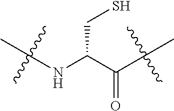 [SMILES: *N[C@H](CS)C(*)=O] |
| dGlu (or dE) | 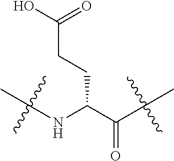 [SMILES: *N[C@H](CCC(=O)O)C(*)=O] |
| dLys (or dK) | 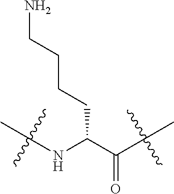 [SMILES: *N[C@H](CCCCN)C(*)=O] |
| dLys(dGlu-DOTA) | 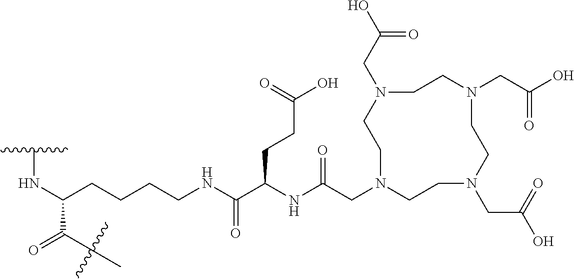 [SMILES: *N[C@H](CCCCNC(=O)[C@@H](CCC(=O)O)NC(=O)CN1CCN(CC(=O)O)CCN(CC(=O)O)CCN(CC(=O)O)CC1)C(*)=O] |
| dLys(DOTA) | 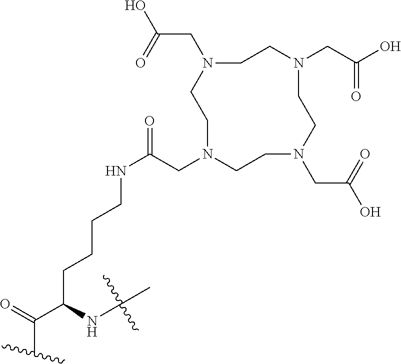 [SMILES: *N[C@H](CCCCNC(=O)CN1CCN(CC(=O)O)CCN(CC(=O)O)CCN(CC(=O)O)CC1)C(*)=O] |
| dLys(gE-DOTA) | 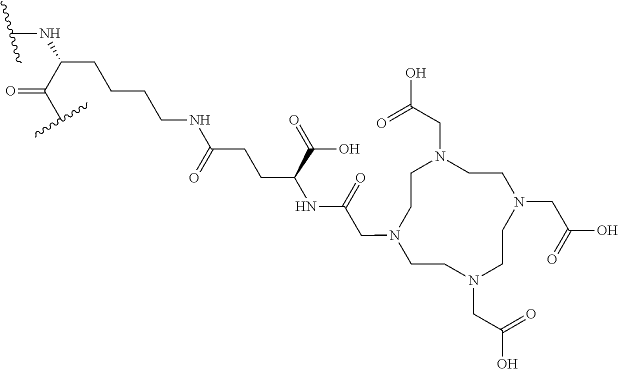 [SMILES: *N[C@H](CCCCNC(=O)CC[C@H](NC(=O)CN1CCN(CC(=O)O)CCN(CC(=O)O)CCN(CC(=O)O)CC1)C(=O)O)C(*)=O] |
| dLys(gE- gE-DOTA) | 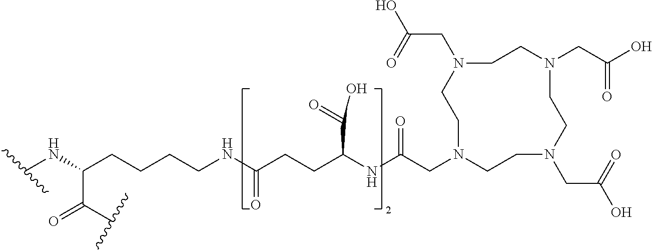 |
| dLys(gE-gE- gE-DOTA) | 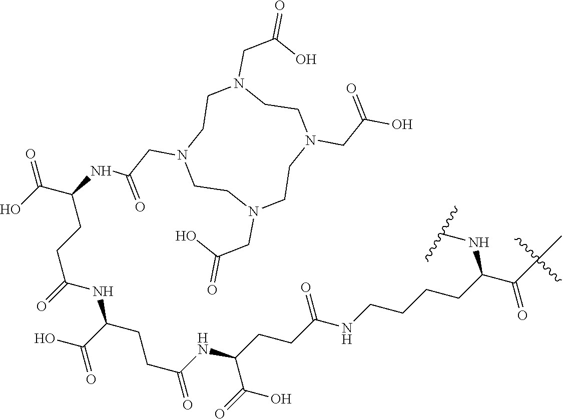 [SMILES: *N[C@H](CCCCNC(=O)CC[C@H](NC(=O)CC[C@H](NC(=O)CC[C@H](NC(=O)CN1CCN(CC(=O)O)CCN(CC(=O)O)CCN(CC(=O)O)CC1)C(=O)O)C(=O)O)C(=O)O)C(*)=O] |
| dLys(gE- tranexamate- DOTA) | 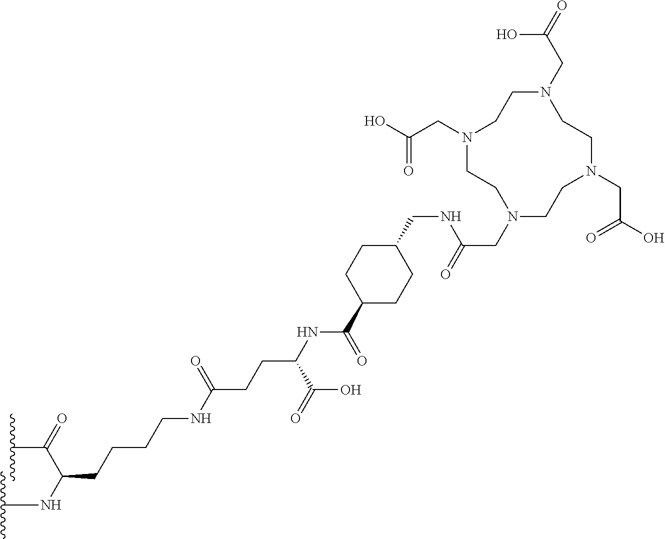 [SMILES: *N[C@H](CCCCNC(=O)CC[C@H](NC(=O)[C@H]1CC[C@H](CNC(=O)CN2CCN(CC(=O)O)CCN(CC(=O)O)CCN(CC(=O)O)CC2)CC1)C(=O)O)C(*)=O] |
| dLys(gGlu-DOTA) | 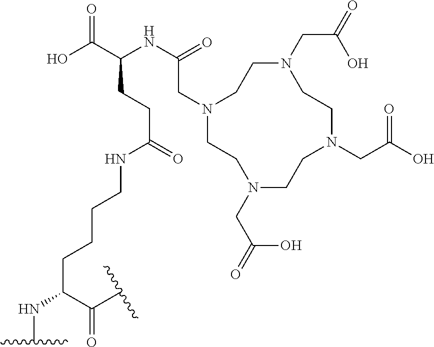 [SMILES: *N[C@H](CCCCNC(=O)CC[C@H](NC(=O)CN1CCN(CC(=O)O)CCN(CC(=O)O)CCN(CC(=O)O)CC1)C(=O)O)C(*)=O] |
| dLys(Glu-DOTA) | 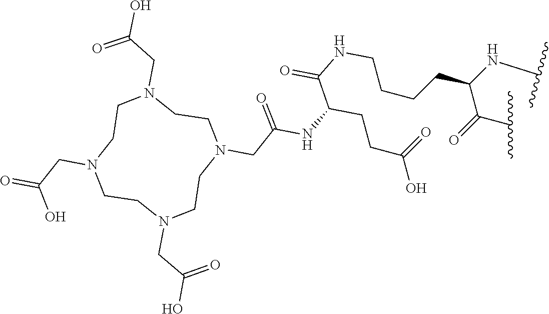 [SMILES: *N[C@H](CCCCNC(=O)[C@H](CCC(=O)O)NC(=O)CN1CCN(CC(=O)O)CCN(CC(=O)O)CCN(CC(=O)O)CC1)C(*)=O] |
| dLys(hGlu-DOTA) | 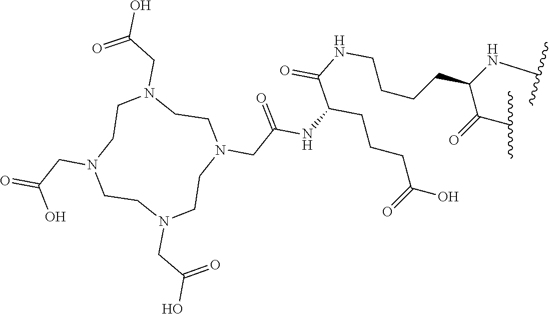 [SMILES: *N[C@H](CCCCNC(=O)[C@H](CCCC(=O)O)NC(=O)CN1CCN(CC(=O)O)CCN(CC(=O)O)CCN(CC(=O)O)CC1)C(*)=O] |
| dLys(hGlu- hGlu-DOTA) | 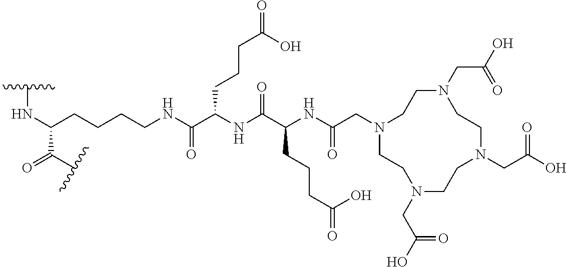 [SMILES: *N[C@H](CCCCNC(=O)[C@H](CCCC(=O)O)NC(=O)[C@H](CCCC(=O)O)NC(=O)CN1CCN(CC(=O)O)CCN(CC(=O)O)CCN(CC(=O)O)CC1)C(*)=O] |
| dLys(hGlu-hGlu- hGlu-DOTA) | 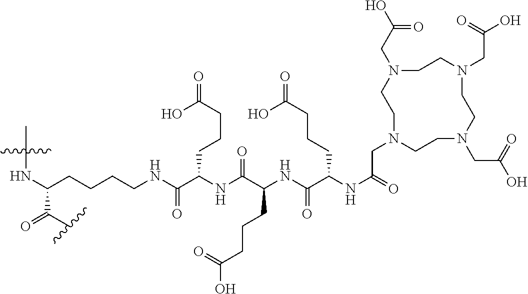 [SMILES: *N[C@H](CCCCNC(=O)[C@H](CCCC(=O)O)NC(=O)[C@H](CCCC(=O)O)NC(=O)[C@H](CCCC(=O)O)NC(=O)CN1CCN(CC(=O)O)CCN(CC(=O)O)CCN(CC(=O)O)CC1)C(*)=O] |
| dLys(hGlu- transhexamate- DOTA) | 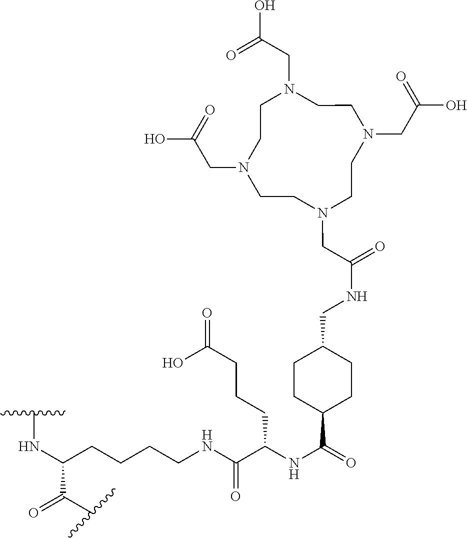 [SMILES: *N[C@H](CCCCNC(=O)[C@H](CCCC(=O)O)NC(=O)[C@H]1CC[C@H](CNC(=O)CN2CCN(CC(=O)O)CCN(CC(=O)O)CCN(CC(=O)O)CC2)CC1)C(*)=O] |
| dLys(PEG24- Lys(DOTA)- gGlu-palmitoyl) | 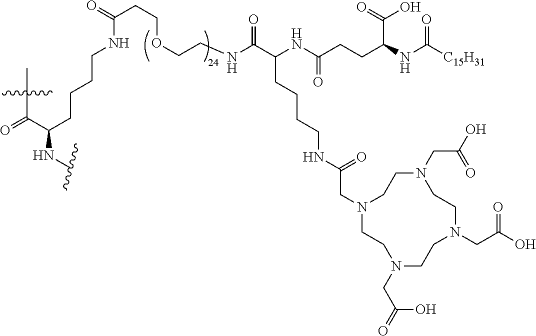 |
| dLys(PEG4- DOTA) | 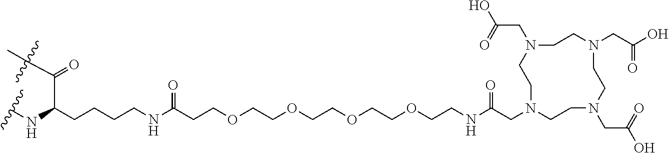 [SMILES: *N[C@H](CCCCNC(=O)CCOCCOCCOCCOCCNC(=O)CN1CCN(CC(=O)O)CCN(CC(=O)O)CCN(CC(=O)O)CC1)C(*)=O] |
| dLys(PEG4- Lys(DOTA)- gGlu-palmitoyl) | 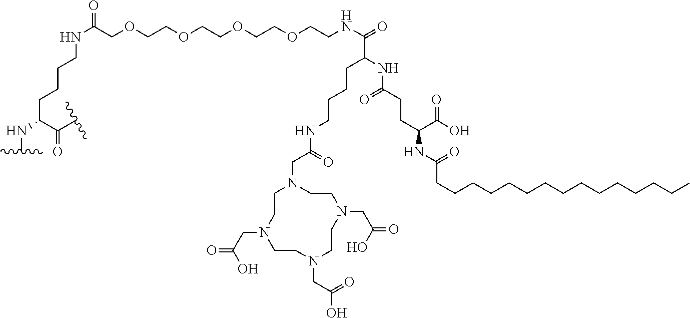 [SMILES: *N[C@H](CCCCNC(=O)COCCOCCOCCOCCNC(=O)C(CCCCNC(=O)CN1CCN(CC(=O)O)CCN(CC(=O)O)CCN(CC(=O)O)CC1)NC(=O)CC[C@H](NC(=O)CCCCCCCCCCCCCCC)C(=O)O)C(*)=O] |
| dLys(PEG8- DOTA) | 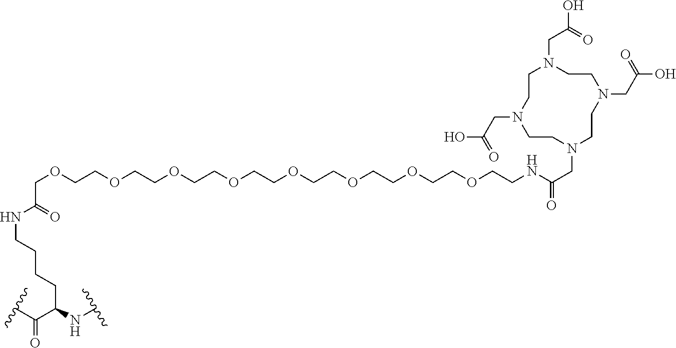 [SMILES: *N[C@H](CCCCNC(=O)COCCOCCOCCOCCOCCOCCOCCOCCNC(=O)CN1CCN(CC(=O)O)CCN(CC(=O)O)CCN(CC(=O)O)CC1)C(*)=O] |
| dLys(R-DOTAGA) | 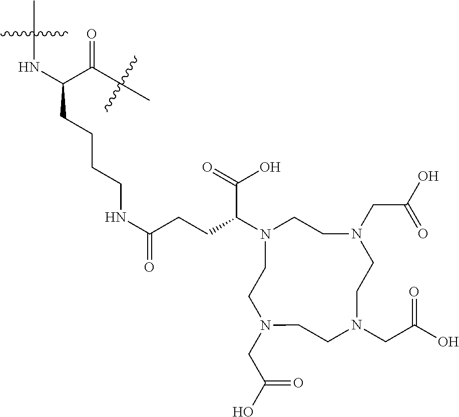 [SMILES: *N[C@H](CCCCNC(=O)CC[C@H](C(=O)O)N1CCN(CC(=O)O)CCN(CC(=O)O)CCN(CC(=O)O)CC1)C(*)=O] |
| dLys(S-DOTAGA) | 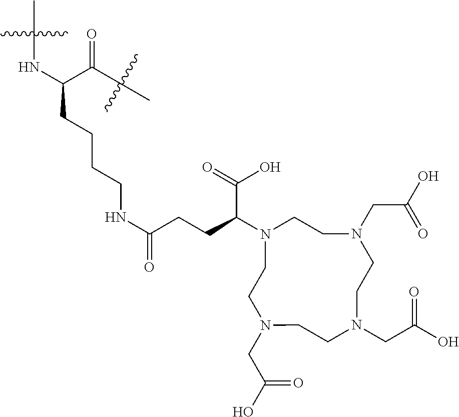 [SMILES: *N[C@H](CCCCNC(=O)CC[C@@H](C(=O)O)N1CCN(CC(=O)O)CCN(CC(=O)O)CCN(CC(=O)O)CC1)C(*)=O] |
| AEA | 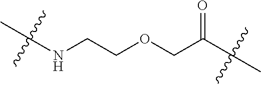 [SMILES: *NCCOCC(*)=O] |
| PEG1 | 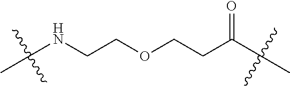 [SMILES: *NCCOCCC(*)=O] |
| PEG4 | 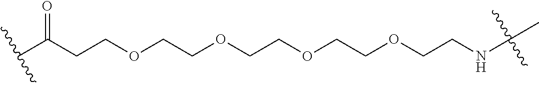 [SMILES: *NCCOCCOCCOCCOCCC(*)=O] |
| PEG8 | 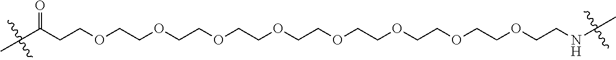 [SMILES: *NCCOCCOCCOCCOCCOCCOCCOCCOCCC(*)=O] |
| dPro (or dP) | 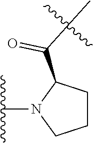 [SMILES: *C(=O)[C@H]1CCCN1*] |
| dSer (or dS) | 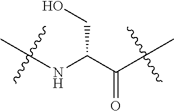 [SMILES: *N[C@H](CO)C(*)=O] |
| dThr (or dT) | 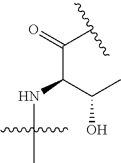 [SMILES: *N[C@@H](C(*)=O)[C@H](C)O] |
| E (or Glu) | 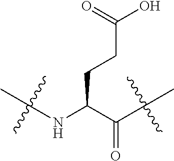 [SMILES: *N[C@@H](CCC(=O)O)C(*)=O] |
| F (or Phe) | 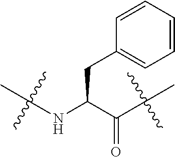 [SMILES: *N[C@@H](Cc1ccccc1)C(*)=O] |
| FSY | 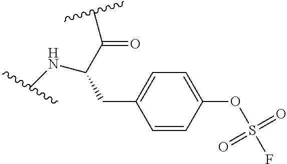 [SMILES: *N[C@@H](Cc1ccc(OS(=O)(=O)F)cc1)C(*)=O] |
| G (or Gly) | 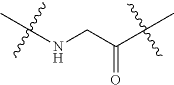 [SMILES: *NCC(*)=O] |
| gE | 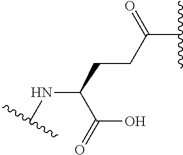 [SMILES: *N[C@@H](CCC(*)=O)C(=O)O] |
| Glu[εLys(OH)-Val- Met-AmBz-DOTA] | 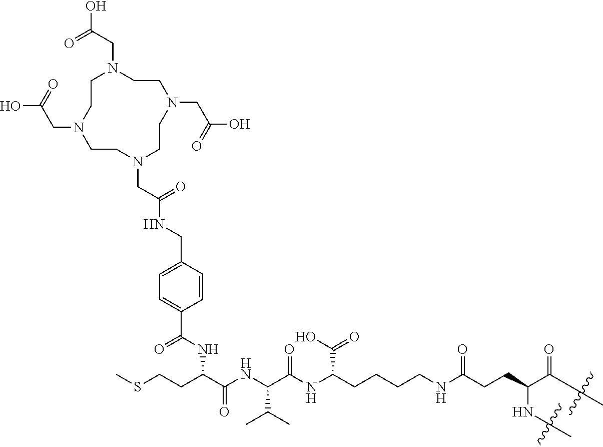 [SMILES: *N[C@@H](CCC(=O)NCCCC[C@H](NC(=O)[C@@H](NC(=O)[C@H](CCSC)NC(=O)c1ccc(CNC(=O)CN2CCN(CC(=O)O)CCN(CC(=O)O)CCN(CC(=O)O)CC2)cc1)C(C)C)C(=O)O)C(*)=O] |
| hC (or homoCys) | 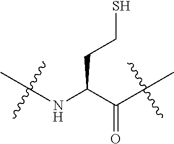 [SMILES: *N[C@@H](CCS)C(*)=O] |
| hCba (or homoCba) | 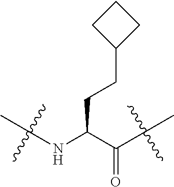 [SMILES: *N[C@@H](CCC1CCC1)C(*)=O] |
| hCha (or homoCha) | 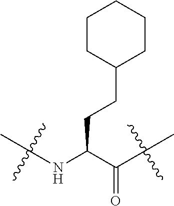 [SMILES: *N[C@@H](CCC1CCCCC1)C(*)=O] |
| hF (or homoPhe) | 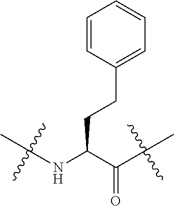 [SMILES: *N[C@@H](CCc1ccccc1)C(*)=O] |
| His (or H) | 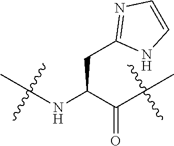 [SMILES: *N[C@@H](Cc1ncc[nH]1)C(*)=O] |
| hLeu (or homoLeu) | 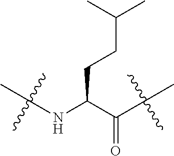 [SMILES: *N[C@@H](CCC(C)C)C(*)=O] |
| HomoArg (or hArg) | 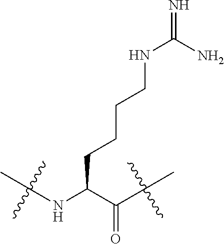 [SMILES: *N[C@@H](CCCCNC(=N)N)C(*)=O] |
| HomoGlu (or hGlu) | 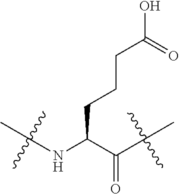 [SMILES: *N[C@@H](CCCC(=O)O)C(*)=O] |
| hSer (or homoSer) | 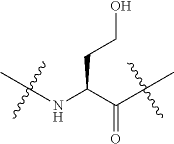 [SMILES: *N[C@@H](CCO)C(*)=O] |
| hTba (or homoTba) | 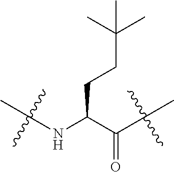 [SMILES: *N[C@@H](CCC(C)(C)C)C(*)=O] |
| hTyr (or homoTyr) | 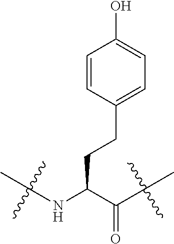 [SMILES: *N[C@@H](CCc1ccc(O)cc1)C(*)=O] |
| hydroxyVal (or hyVal) | 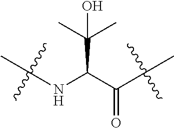 [SMILES: *N[C@H](C(*)=O)C(C)(C)O] |
| Ile | 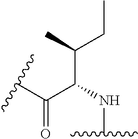 [SMILES: *N[C@H](C(*)=O)[C@@H](C)CC] |
| K(PEG21-COOH) | 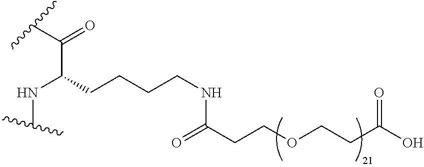 |
| K(yE-C16OH) | 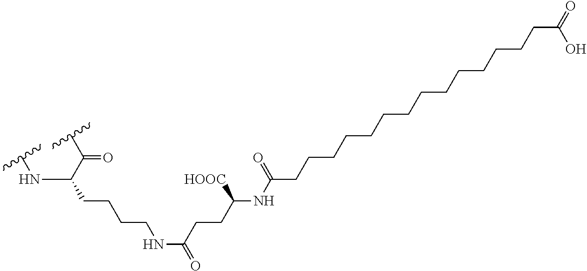 [SMILES: *N[C@@H](CCCCNC(=O)CC[C@H](NC(=O)CCCCCCCCCCCCCCC(=O)O)C(=O)O)C(*)=O] |
| K(yE-yE- yE-C18OH) | 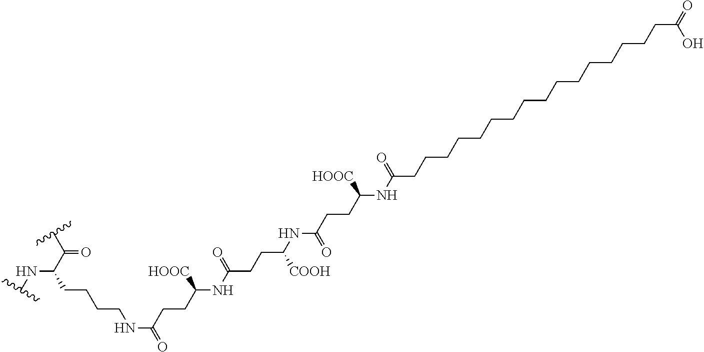 [SMILES: *N[C@@H](CCCCNC(=O)CC[C@H](NC(=O)CC[C@H](NC(=O)CC[C@H](NC(=O)CCCCCCCCCCCCCCCCC(=O)O)C(=O)O)C(=O)O)C(=O)O)C(*)=O] |
| L (or Leu) | 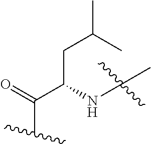 [SMILES: *N[C@@H](CC(C)C)C(*)=O] |
| K (or Lys) | 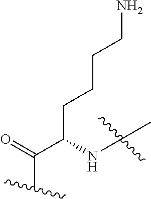 [SMILES: *N[C@@H](CCCCN)C(*)=O] |
| Lys(Ac) | 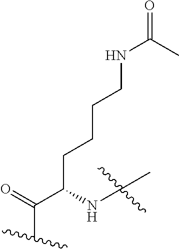 [SMILES: *N[C@@H](CCCCNC(C)=O)C(*)=O] |
| Lys(DOTA) | 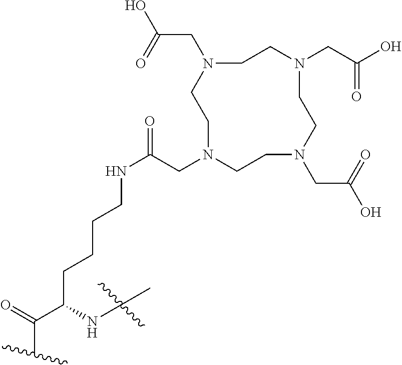 [SMILES: *N[C@@H](CCCCNC(=O)CN1CCN(CC(=O)O)CCN(CC(=O)O)CCN(CC(=O)O)CC1)C(*)=O] |
| Lys(gE-DOTA) | 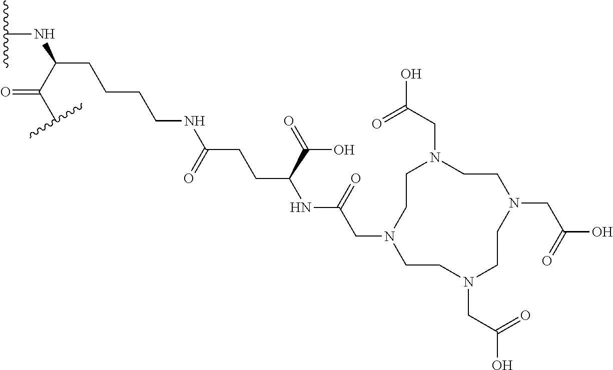 [SMILES: *N[C@@H](CCCCNC(=O)CC[C@H](NC(=O)CN1CCN(CC(=O)O)CCN(CC(=O)O)CCN(CC(=O)O)CC1)C(=O)O)C(*)=O] |
| Lys(gE-gE-DOTA) | 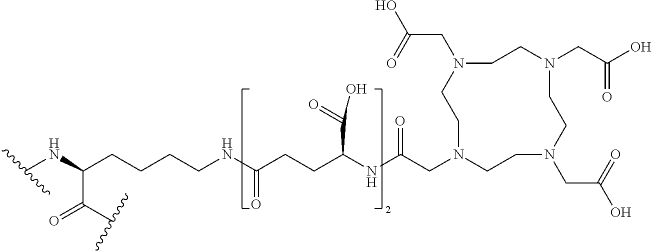 |
| Lys(Me)2 | 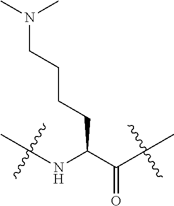 [SMILES: *N[C@@H](CCCCN(C)C)C(*)=O] |
| Lys(Me)3 | 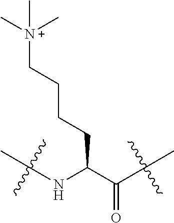 [SMILES: *N[C@@H](CCCC[N+](C)(C)C)C(*)=O] |
| Lys(PEG4-DOTA) | 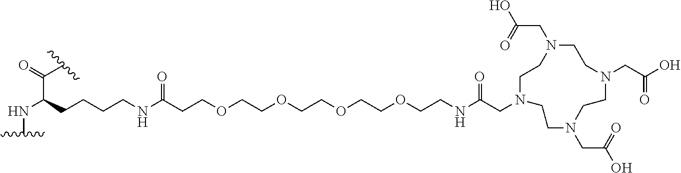 [SMILES: *N[C@H](CCCCNC(=O)CCOCCOCCOCCOCCNC(=O)CN1CCN(CC(=O)O)CCN(CC(=O)O)CCN(CC(=O)O)CC1)C(*)=O] |
| M (or Met) | 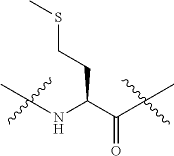 [SMILES: *N[C@@H](CCSC)C(*)=O] |
| MAF(DOTA) | 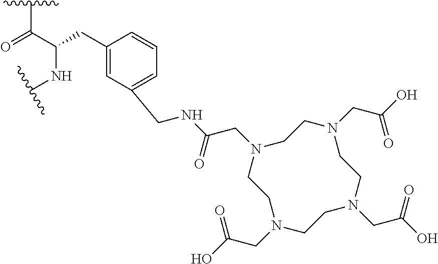 [SMILES: *N[C@@H](Cc1cccc(CNC(=O)CN2CCN(CC(=O)O)CCN(CC(=O)O)CCN(CC(=O)O)CC2)c1)C(*)=O] |
| MAF(gE-DOTA) | 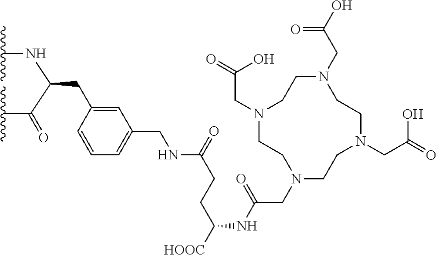 [SMILES: *N[C@@H](Cc1cccc(CNC(=O)CC[C@H](NC(=O)CN2CCN(CC(=O)O)CCN(CC(=O)O)CCN(CC(=O)O)CC2)C(=O)O)c1)C(*)=O] |
| MAF(gE- gE-DOTA) | 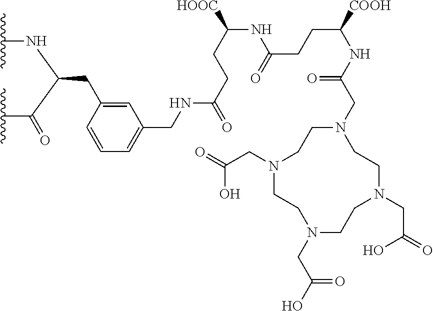 [SMILES: *N[C@@H](Cc1cccc(CNC(=O)CC[C@H](NC(=O)CC[C@H](NC(=O)CN2CCN(CC(=O)O)CCN(CC(=O)O)CCN(CC(=O)O)CC2)C(=O)O)C(=O)O)c1)C(*)=O] |
| MAF(gE-gE- gE-DOTA) | 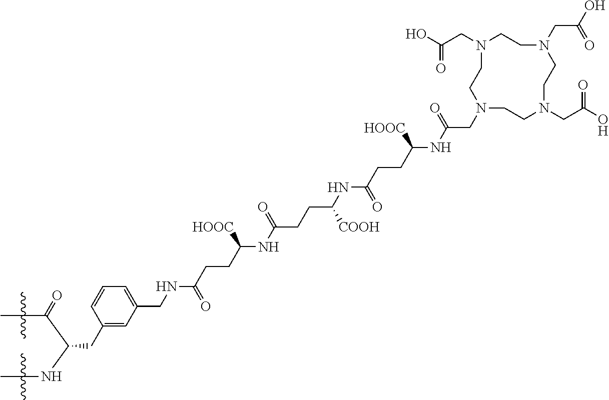 [SMILES: *N[C@@H](Cc1cccc(CNC(=O)CC[C@H](NC(=O)CC[C@H](NC(=O)CC[C@H](NC(=O)CN2CCN(CC(=O)O)CCN(CC(=O)O)CCN(CC(=O)O)CC2)C(=O)O)C(=O)O)C(=O)O)c1)C(*)=O] |
| MAF(PEG4- DOTA) | 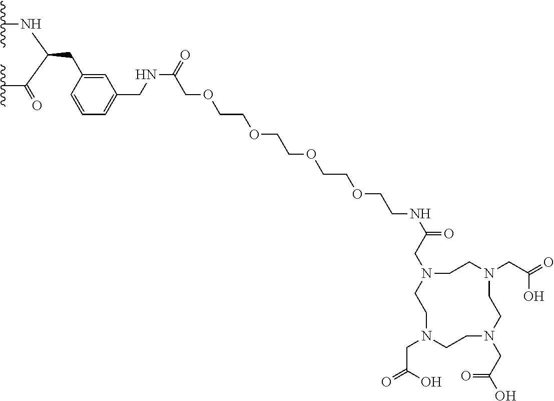 [SMILES: *N[C@@H](Cc1cccc(CNC(=O)COCCOCCOCCOCCNC(=O)CN2CCN(CC(=O)O)CCN(CC(=O)O)CCN(CC(=O)O)CC2)c1)C(*)=O] |
| MAF(PEG8- DOTA) | 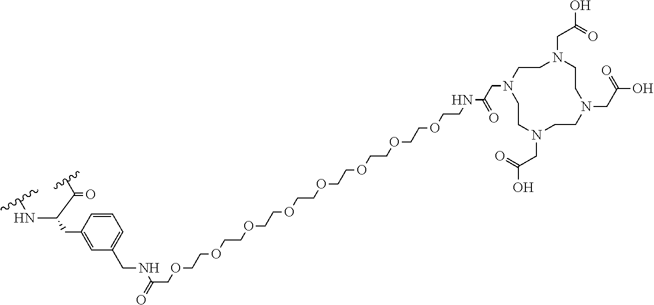 [SMILES: *N[C@@H](Cc1cccc(CNC(=O)COCCOCCOCCOCCOCCOCCOCCOCCNC(=O)CN2CCN(CC(=O)O)CCN(CC(=O)O)CCN(CC(=O)O)CC2)c1)C(*)=O] |
| N (or Gln) | 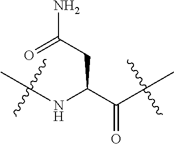 [SMILES: *N[C@@H](CC(N)=O)C(*)=O] |
| N(CH2)2COOHGly | 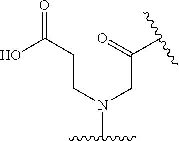 [SMILES: *C(=O)CN(*)CCC(=O)O] |
| NetGly | 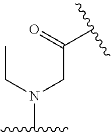 [SMILES: *C(=O)CN(*)CC] |
| Nle | 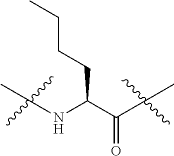 [SMILES: *N[C@@H](CCCC)C(*)=O] |
| NMeCys | 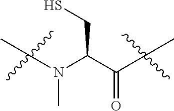 [SMILES: *C(=O)[C@H](CS)N(*)C] |
| NMeIle | 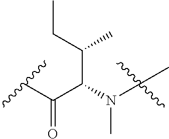 [SMILES: *C(=O)[C@H]([C@@H](C)CC)N(*)C] |
| NMeLys | 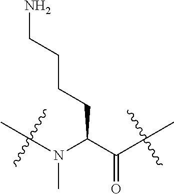 [SMILES: *C(=O)[C@H](CCCCN)N(*)C] |
| NMeNle | 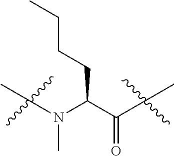 [SMILES: *C(=O)[C@H](CCCC)N(*)C] |
| NMePhe | 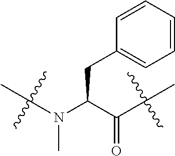 [SMILES: *C(=O)[C@H](Cc1ccccc1)N(*)C] |
| NMeSer | 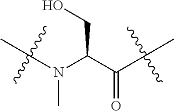 [SMILES: *C(=O)[C@H](CO)N(*)C] |
| N-Me-ThpA | 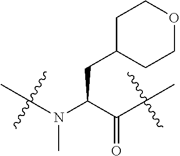 [SMILES: *C(=O)[C@H](CC1CCOCC1)N(*)C] |
| NMeTyr | 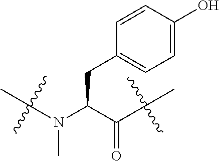 [SMILES: *C(=O)[C@H](Cc1ccc(O)cc1)N(*)C] |
| NMeVal | 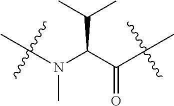 [SMILES: *C(=O)[C@H](C(C)C)N(*)C] |
| Nva | 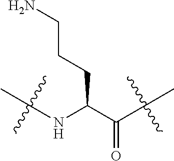 [SMILES: *N[C@@H](CCCN)C(*)=O] |
| OMeTyr | 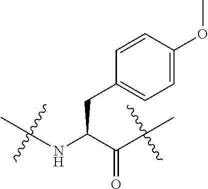 [SMILES: *N[C@@H](Cc1ccc(OC)cc1)C(*)=O] |
| Orn | 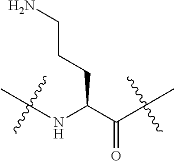 [SMILES: *N[C@@H](CCCN)C(*)=O] |
| P (or Pro) | 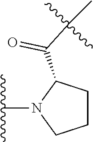 [SMILES: *C(=O)[C@@H]1CCCN1*] |
| Pen | 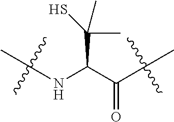 [SMILES: *N[C@H](C(*)=O)C(C)(C)S] |
| Pip (or Pip acid) | 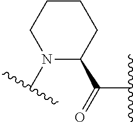 [SMILES: *C(=O)[C@@H]1CCCCN1*] |
| Pip(Ac)Ala | 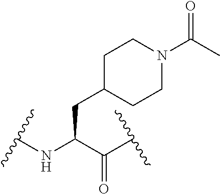 [SMILES: *N[C@@H](CC1CCN(C(C)=O)CC1)C(*)=O] |
| Pip(C4diacid)Ala | 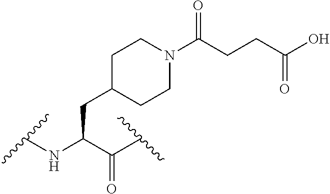 [SMILES: *N[C@@H](CC1CCN(C(=O)CCC(=O)O)CC1)C(*)=O] |
| Pip(CH2CO2H) Ala or Pip(CH2COOH) Ala | 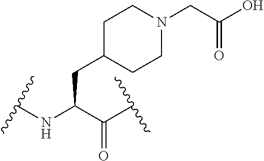 [SMILES: *N[C@@H](CC1CCN(CC(=O)O)CC1)C(*)=O] |
| Pip(CONH2)Ala | 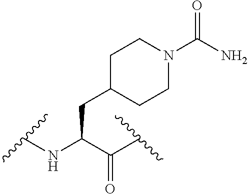 [SMILES: *N[C@@H](CC1CCN(C(N)=O)CC1)C(*)=O] |
| Pip(DOTA)Ala | 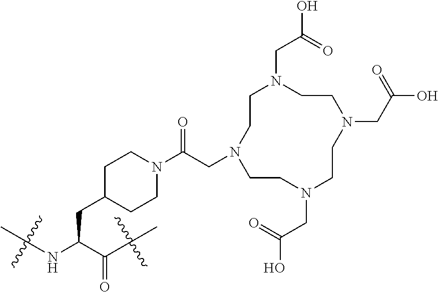 [SMILES: *N[C@@H](CC1CCN(C(=O)CN2CCN(CC(=O)O)CCN(CC(=O)O)CCN(CC(=O)O)CC2)CC1)C(*)=O] |
| Pip(gE-DOTA)Ala | 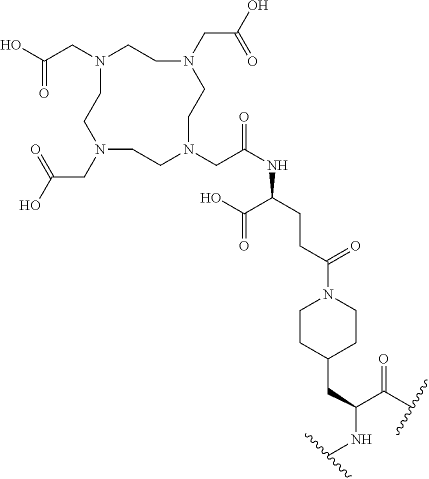 [SMILES: *N[C@@H](CC1CCN(C(=O)CC[C@H](NC(=O)CN2CCN(CC(=O)O)CCN(CC(=O)O)CCN(CC(=O)O)CC2)C(=O)O)CC1)C(*)=O] |
| Pip(gE-gE- DOTA)Ala | 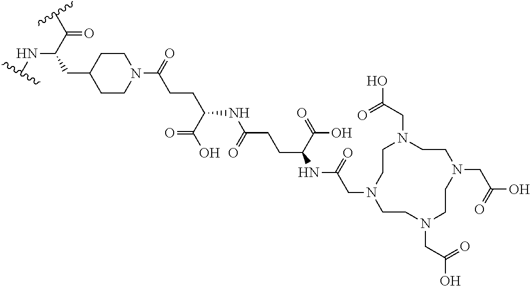 [SMILES: *N[C@@H](CC1CCN(C(=O)CC[C@H](NC(=O)CC[C@H](NC(=O)CN2CCN(CC(=O)O)CCN(CC(=O)O)CCN(CC(=O)O)CC2)C(=O)O)C(=O)O)CC1)C(*)=O] |
| Pip(Glu-DOTA)Ala | 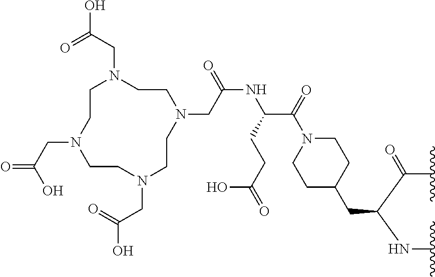 [SMILES: *N[C@@H](CC1CCN(C(=O)[C@H](CCC(=O)O)NC(=O)CN2CCN(CC(=O)O)CCN(CC(=O)O)CCN(CC(=O)O)CC2)CC1)C(*)=O] |
| Pip(Me)Ala | 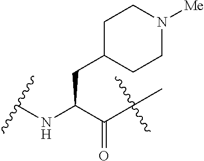 [SMILES: *N[C@@H](CC1CCN(C)CC1)C(*)=O] |
| Pip(PEG2- DOTA)Ala | 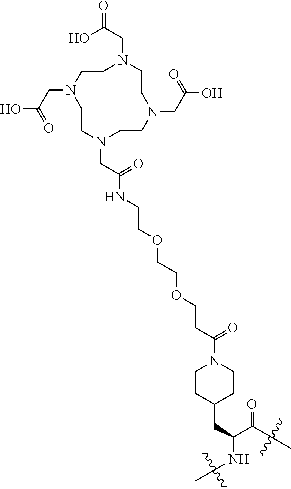 [SMILES: *N[C@@H](CC1CCN(C(=O)CCOCCOCCNC(=O)CN2CCN(CC(=O)O)CCN(CC(=O)O)CCN(CC(=O)O)CC2)CC1)C(*)=O] |
| Pip(PEG4-AB)Ala | 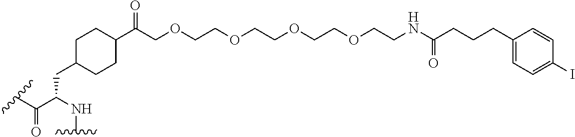 [SMILES: *N[C@@H](CC1CCC(C(=O)COCCOCCOCCOCCNC(=O)CCCc2ccc(I)cc2)CC1)C(*)=O] |
| Pip(PEG4-Ac)Ala | 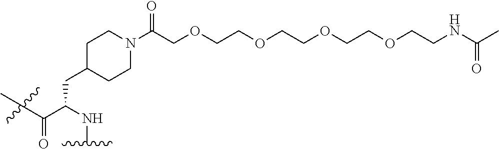 [SMILES: *N[C@@H](CC1CCN(C(=O)COCCOCCOCCOCCNC(C)=O)CC1)C(*)=O] |
| Pip(PEG4- C4diacid)Ala | 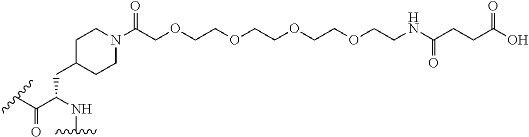 [SMILES: *N[C@@H](CC1CCN(C(=O)COCCOCCOCCOCCNC(=O)CCC(=O)O)CC1)C(*)=O] |
| Pip(PEG4- DOTA)Ala | 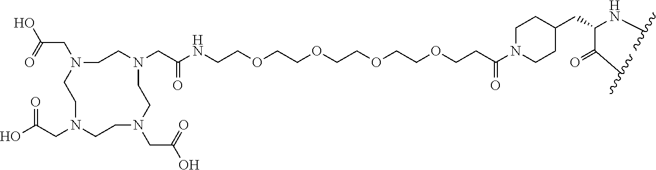 [SMILES: *N[C@@H](CC1CCN(C(=O)CCOCCOCCOCCOCCNC(=O)CN2CCN(CC(=O)O)CCN(CC(=O)O)CCN(CC(=O)O)CC2)CC1)C(*)=O] |
| Pip(R- DOTAGA)Ala | 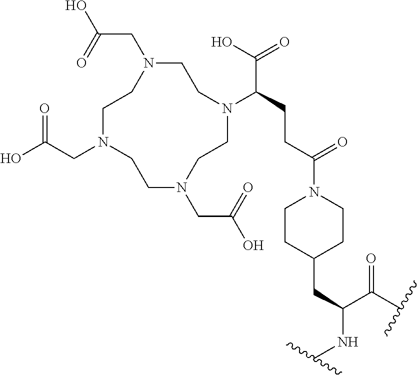 [SMILES: *N[C@@H](CC1CCN(C(=O)CC[C@H](C(=O)O)N2CCN(CC(=O)O)CCN(CC(=O)O)CCN(CC(=O)O)CC2)CC1)C(*)=O] |
| PipAla | 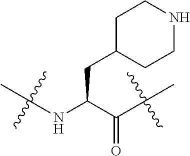 [SMILES: *N[C@@H](CC1CCNCC1)C(*)=O] |
| Pyroglutamate | 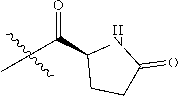 [SMILES: *C(=O)[C@@H]1CCC(=O)N1] |
| S (or Ser) | 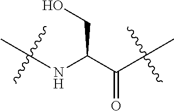 [SMILES: *N[C@@H](CO)C(*)=O] |
| S4H | 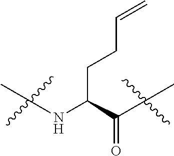 [SMILES: *N[C@@H](CCC=C)C(*)=O] |
| Sar | 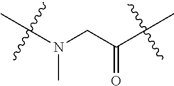 [SMILES: *C(=O)CN(*)C] |
| T (or Thr) | 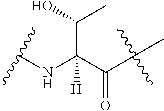 [SMILES: *N[C@H](C(*)=O)[C@@H](C)O] |
| Tbg | 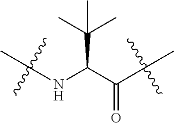 [SMILES: *N[C@H](C(*)=O)C(C)(C)C] |
| Tba (or tBuAla) | 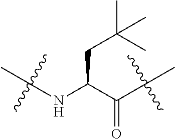 [SMILES: *N[C@@H](CC(C)(C)C)C(*)=O] |
| ThpA | 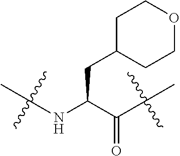 [SMILES: *N[C@@H](CC1CCOCC1)C(*)=O] |
| ThpG | 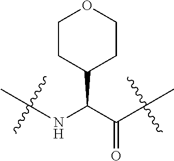 [SMILES: *N[C@H](C(*)=O)C1CCOCC1] |
| trans4FPro (or trans4Fluoro-Pro) | 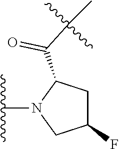 [SMILES: *C(=O)[C@@H]1C[C@@H](F)CN1*] |
| transHyp (or trans4OH-Pro) | 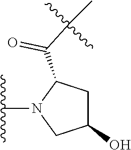 [SMILES: *C(=O)[C@@H]1C[C@@H](O)CN1*] |
| 5,5-diMe-Pro (or 5,5-dimethyl-P) | 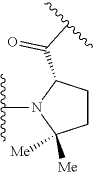 |
| Trp(CH2COOH) | 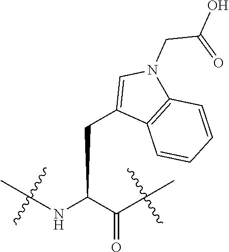 [SMILES: *N[C@@H](Cc1cn(CC(=O)O)c2ccccc12)C(*)=O] |
| Trp(Me) | 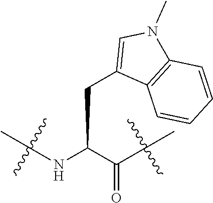 [SMILES: *N[C@@H](Cc1cn(C)c2ccccc12)C(*)=O] |
| V (or Val) | 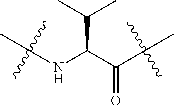 [SMILES: *N[C@H](C(*)=O)C(C)C] |
| W (or Trp) | 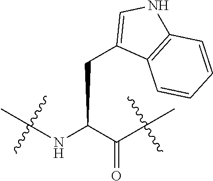 [SMILES: *N[C@@H](Cc1c[nH]c2ccccc12)C(*)=O] |
| Y (or Tyr) | 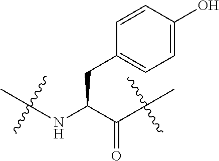 [SMILES: *N[C@@H](Cc1ccc(O)cc1)C(*)=O] |
| εLys(Val- Met-AmBz- DOTA)-COOH | 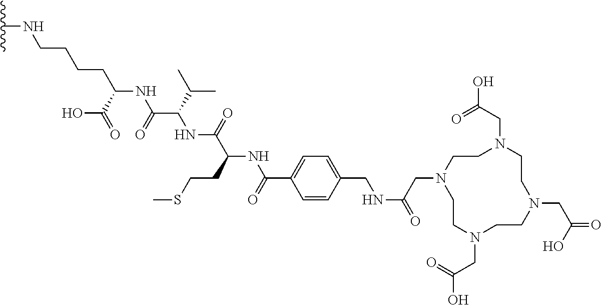 [SMILES: *NCCCC[C@H](NC(=O)[C@@H](NC(=O)[C@H](CCSC)NC(=O)c1ccc(CNC(=O)CN2CCN(CC(=O)O)CCN(CC(=O)O)CCN(CC(=O)O)CC2)cc1)C(C)C)C(=O)O] |
| DOTA | 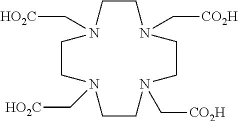 [SMILES: O=C(O)CN1CCN(CC(=O)O)CCN(CC(=O)O)CCN(CC(=O)O)CC1] |

### V. Equivalents and Scope

While various disclosure embodiments have been particularly shown and described in the present disclosure, it will be understood by those skilled in the art that various changes in form and details may be made without departing from the spirit and scope of the embodiments disclosed herein and set forth in the appended claims.

Those skilled in the art will recognize or be able to ascertain using no more than routine experimentation, many equivalents to the specific embodiments described herein. The scope of the present disclosure is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of a group is present in, employed in, or otherwise relevant to a given product or process. The disclosure includes embodiments in which more than one, or all group members are present in, employed in, or otherwise relevant to a given product or process.

It is also noted that the term “comprising” is intended to be open and permits but does not require the inclusion of additional elements or steps. When the term “comprising” is used herein, the terms “consisting of” and “or including” are thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to those of ordinary skill in the art, they may be excluded even if the exclusion is not set forth explicitly herein. Any particular embodiments of compositions disclosed herein can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### Examples

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Reagents—Abbreviations

ACN (or MeCN): Acetonitrile
AC₂O: Acetic anhydride
AcOH: Acetic acid
Boc: Tert-butoxy-carbonyl
DCM: Dichloromethane
DIC: N,N′-Diisopropylcarbodiimide
DIPEA: N,N-Diisopropylethylamine
DMF: N,N-Dimethylformamide
DMSO: Dimethyl sulfoxide
Et₂O: Diethyl ether
Fmoc: Fluorenylmethyloxycarbonyl
HATU: 2-(3H-[1,2,3]triazolo[4,5-b]pyridin-3-yl)-1,1,3,3-tetramethylisouronium
HOBt: 1-Hydroxybenzotriazole
MeOH: Methanol
Mpe: 3-Methylpentan-3-yl
MTBE: Methyl-tert-butyl-ether
NMP: N-Methyl-2-Pyrrolidone
Oxyma (or OxymaPure): Ethyl cyanohydroxyiminoacetate
Dde: 1-(4,4-dimethyl-2,6-dioxocyclohexylidene)ethyl
TFA: Trifluoroacetic acid
TIPS: Triisopropylsilane
Cp*RuCl(PPh₃)₂: Chloro(pentamethylcyclopentadienyl)bis(triphenylphosphine)ruthenium (II) Grubbs2: (1,3-Dimesitylimidazolidin-2-ylidene)(tricyclohexylphosphine)benzylidene ruthenium dichloride

### Amino Acids and Building Blocks

Na-(((9H-fluoren-9-yl)methoxy)carbonyl)-1-(tert-butoxycarbonyl)-L-tryptophan (W)
(((9H-fluoren-9-yl)methoxy)carbonyl)-L-threonine (T)
(((9H-fluoren-9-yl)methoxy)carbonyl)-L-isoleucine (I)
N-(((9H-fluoren-9-yl)methoxy)carbonyl)-S-trityl-L-cysteine (C)
(S)-2-((((9H-fluoren-9-yl)methoxy)carbonyl)amino)-3-(tritylamino)propanoic acid (N)
Na-(((9H-fluoren-9-yl)methoxy)carbonyl)-Np-(tert-butoxycarbonyl)-L-histidine (H)
(S)-2-((((9H-fluoren-9-yl)methoxy)carbonyl)amino)-4-((3-methylpentan-3-yl)oxy)-4-oxobutanoic acid (D).
(((9H-fluoren-9-yl)methoxy)carbonyl)-L-proline (P)
N2-(((9H-fluoren-9-yl)methoxy)carbonyl)-N6-(1-(4,4-dimethyl-2,6-dioxocyclohexylidene)ethyl)-L-lysine (K(Dde))
N2-(((9H-fluoren-9-yl)methoxy)carbonyl)-N6-(1-(4,4-dimethyl-2,6-dioxocyclohexylidene)ethyl)-D-lysine (D-Lys(Dde))
N2-(((9H-fluoren-9-yl)methoxy)carbonyl)-N6-(tert-butoxycarbonyl)-D-lysine (D-Lys(Boc))
(S)-2-((((9H-fluoren-9-yl)methoxy)carbonyl)amino)-3-(naphthalen-2-yl)propanoic acid (2Nal).
2-(4,7,10-tris(2-(tert-butoxy)-2-oxoethyl)-1,4,7,10-tetraazacyclododecan-1-yl)acetic acid (DOTA(tBu)₃).
2,2′,2″-(10-(2-((2,5-dioxopyrrolidin-1-yl)oxy)-2-oxoethyl)-1,4,7,10-tetraazacyclododecane-1,4,7-triyl)triacetic acid (DOTA-NHS)
(S)-4-((((9H-fluoren-9-yl)methoxy)carbonyl)amino)-6,6-dimethyl-5-oxoheptanoic acid (gE).
3-((tert-butoxycarbonyl)amino)propanoic acid (Boc-betaAla)
(S)-2-((((9H-fluoren-9-yl)methoxy)carbonyl)amino) pent-4-enoic acid (AllyIG)
(S)-2-((((9H-fluoren-9-yl)methoxy)carbonyl)amino)-3-azidopropanoic acid (DapN₃).
(S)-2-((((9H-fluoren-9-yl)methoxy)carbonyl)amino) pent-4-ynoic acid (Pra)

### Example 1. Targeting Construct Preparation

A targeting construct is prepared by combining a targeting moiety with a cargo. The targeting moiety incorporates peptide sequences specific for a cancer cell antigen selected from B7-H3. The cargo includes a radioactive agent that includes a radionuclide. The targeting moiety and cargo are combined using a linker.

Chelators such as DOTA can be attached to any place of the cyclic peptide without negatively affecting the binding of the cyclic peptide to its targets. In some embodiments, chelators such as DOTA can be attached directly to the N-terminal amine or to a short linker attached to that same residue. Alternatively, chelators such as DOTA can be attached via a short linker to the C-terminus or to a side chain that can tolerate its presence. In some embodiments, a crosslinker, such as dibromoxilene, that has previously prefunctionlaized with a chelator moiety can be attached to the cyclic peptide.

In some embodiments, DOTA chelators can be attached to the cyclic peptide targeting moieties according to the following general method.

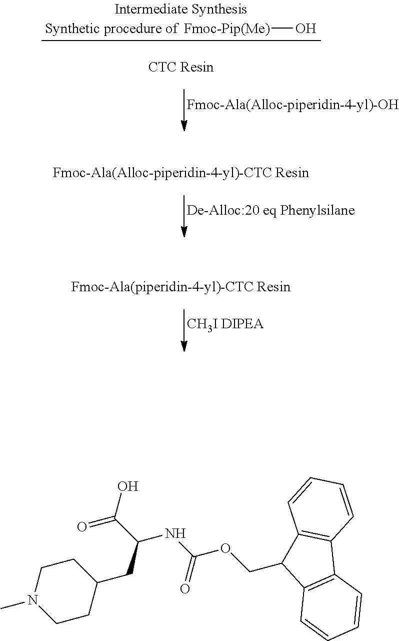

CTC Resin (1.5 g, 1.5 mmol) swelled by DMF (5 mL) for 2 hours was added Fmoc-Ala (Alloc-piperidin-4-yl)-OH (0.287 g, 0.6 mmol) and DIPEA (1.3 mL, 7.5 mmol) sequentially. The mixture was kept at RT for 3 hours while a stream of nitrogen bubbled through it. Then 2 mL MeOH was added into the mixture and resulting mixture was stirred at RT for another 0.5 hours. Then the reaction mixture was filtered, and the resin was washed with DMF (3×15 mL) and MeOH (3×15 mL).

De-Alloc: The peptidyl reisn was first washed with DCM (3 equivalent volume of the resin) for 3 times and then the suspension was filtered. 0.5 equivalent of Pd(PPh₃)₄ (0.233 g), 20 equivalent of phenylsilane (0.985 mL) and 5 mL DCM were added into the resin sequentially and the reaction was carried out at RT for 1.5 hours while a stream of nitrogen bubbled through it, then the suspension was filtered. Usually, the above removal of Alloc procedure was repeated once more to make sure Alloc was removed completely. Finally, the resin was washed with 10 mL solution of 0.25% sodium diethyldithiocarbamatre in DMF (1 g sodium diethyldithiocarbamatre dissolved in 400 mL DMF) for 5 minutes and the washing was repeated 6 times until the resin went back to the original color.

CH₃I (0.056 g, 0.4 mmol) and DIPEA (0.35 mL, 2 mmol) were added into the resin. The mixture was kept at room temperature for 3 hours while a stream of nitrogen bubbled through it. Then resin was washed with DCM (3×15 mL) and MeOH (3×15 mL). The resin was dried under vacuum overnight.

15 mL 1% TFA/DCM was added into peptidyl resin (1.5 g) under the protection of nitrogen. The mixture was shaken for 1 hour at RT. The filtrate was evaporated to get the crude which was purified by RP-HPLC to give the final compound Fmoc-Pip(Me)—OH (23 mg).

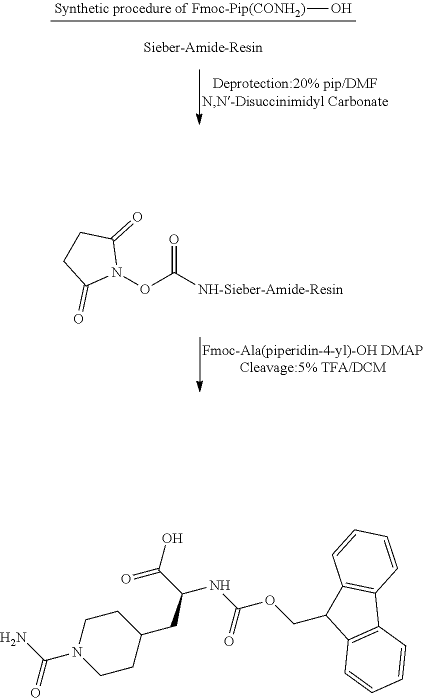

Sieber Amide Resin (0.3 mmol, 0.857 g) swelled in DMF (15 mL) for 2 hours, then 20% piperidine in DMF (15 mL) was added. The mixture was kept at room temperature for 0.5 hours while a stream of nitrogen bubbled through it. The mixture was filtered and the peptidyl resin was washed with DMF (5*15 mL). N,N′-Disuccinimidyl Carbonate (0.9 mmol, 0.23 g), DMF (3 mL), NMM (0.9 mmol, 0.108 mL) and DMAP (0.3 mmol, 0.036 g) were added into the resin sequentially. The suspension was kept at room temperature overnight. The Ninhydrin test indicated the coupling completed. The mixture was filtered and the peptidyl resin was washed with DMF (3*15 mL).

Then Fmoc-Ala (piperidin-4-yl)-OH (0.9 mmol, 0.355 g), NMM (0.9 mmol, 0.108 mL), DMAP (0.3 mmol, 0.036 g) and DMF (3 mL) were added into the resin. The suspension was kept at room temperature for 4 hours while a stream of nitrogen bubbled through it. It was washed with DCM (3*15 mL) and MeOH (3*15 mL). The resin was dried under vacuum overnight.

15 mL 5% TFA/DCM was added into peptidyl resin (1 g) under the protection of nitrogen. The mixture was shaken for 1 hour at RT. The filtrate was evaporated to get the crude which was purified by RP-HPLC to give the final compound Fmoc-Pip(CONH₂)—OH (150 mg).

### Synthetic Procedure of Fmoc-Pip(CH2COOtBu)Ala-OH

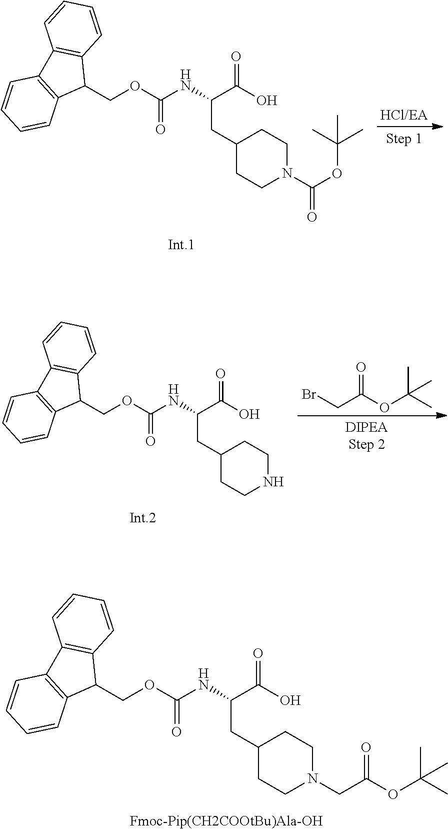

Step1: The flask (100 mL) was charged with Compound Int.1 (1 g, 2.02 mmol). 20 mL 4N HCl/EA was added into the flask and stirred at RT for 2 hours. The reaction was concentrated to dryness and carried over to the next step without further purification.

Step2: The compound Int.2 dissolved by 20 mL ACN/H₂O=1/1 was added tert-Butyl bromoacetate (0.334 mL, 2.22 mmol, 1.1 equiv.) and DIPEA (0.702 mL, 10.1 mmol, 5 equiv.) sequentially. The mixture was stirred for 2 hours at RT. The mixture was filtered with 0.45 μm filter. The filtrate was purified by RP-HPLC. The desired fractions were collected and lyophilized to give the final compound Fmoc-Pip(CH2COOtBu)Ala-OH (0.789 g) as a white solid.

### Synthetic Procedure of Fmoc-Pip(C₄diacid-tBu)—OH

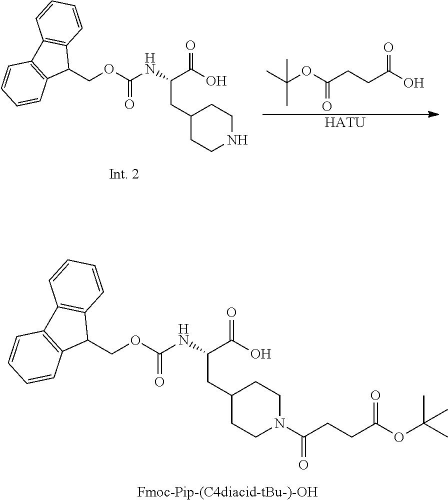

To a solution of Mono-tert-butyl succinate (0.132 g, 0.76 mmol) and HATU (0.289 g, 0.76 mmol) in 2 mL DMF at ice bath was added NMM (0.25 mL, 2.28 mmol) and stirred for 5 minutes. Then, the Compound Int. 2 (300 mg) dissolved by 2 mL DMF was added dropwise to the solution. Upon completion, EA/H₂O (50 mL: 100 mL) was added to the above reaction solution, then 5% H₃PO₄ buffer was added to adjust the pH to 3-4. The EA phase was separated and washed by brine once, then collected and dried by Na₂SO₄. EA solution was evaporated to get the crude which was purified by RP-HPLC to give the final product Fmoc-Pip(C₄diacid-tBu)—OH (199 mg, 95%).

### Synthetic Procedure of Fmoc-Pip(Ac)—OH

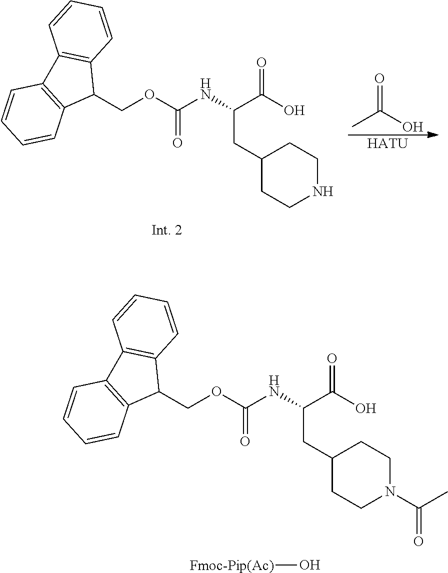

To a solution of AcOH (0.042 g, 0.76 mmol) and HATU (0.289 g, 0.76 mmol) in 2 mL DMF at ice bath was added NMM (0.25 mL, 2.28 mmol) and stirred for 5 minutes. Then, the Compound Int. 2 (300 mg) dissolved by 2 mL DMF was added dropwise to the solution. Upon completion, EA/H₂O (50 mL: 100 mL) was added to the above reaction solution, then 5% H₃PO₄ buffer was added to adjust the pH to 3-4. The EA phase was separated and washed by brine once. The EA phase was collected and dried by Na₂SO₄ and then EA was evaporated to get the crude which was purified by RP-HPLC to give the final product Fmoc-Pip(Ac)—OH (198 mg).

### Synthetic Procedure of Fmoc-(N-Me-ThpA)-OH

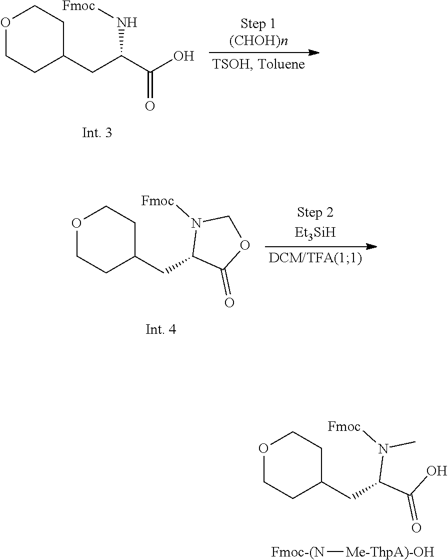

Step1: To a solution of Int. 3 (1 g, 2.53 mmol), paraformaldehyde (3.416 g, 38 mmol) and TSOH (0.085 g, 0.379 mmol) in toluene (15 mL) was refluxed to remove H₂O for 2 hours. The mixture was filtered and the filtrate was washed with brine and dried by Na₂SO₄ to give the Int. 4 (1 g).

Step2: A solution of Int. 4 (1 g) in DCM: TFA (1:1, 20 mL) was added Et₃SiH (2 mL), the solution was stirred at RT overnight. The reaction solution was concentrated and purified by RP-HPLC to give the final compound Fmoc-(N-Me-ThpA)-OH (0.79 g).

### Synthetic Procedure of Fmoc-Ala ([DOTA-tris(tBuester)]-PEG4-piperidin-4-yl)-OH

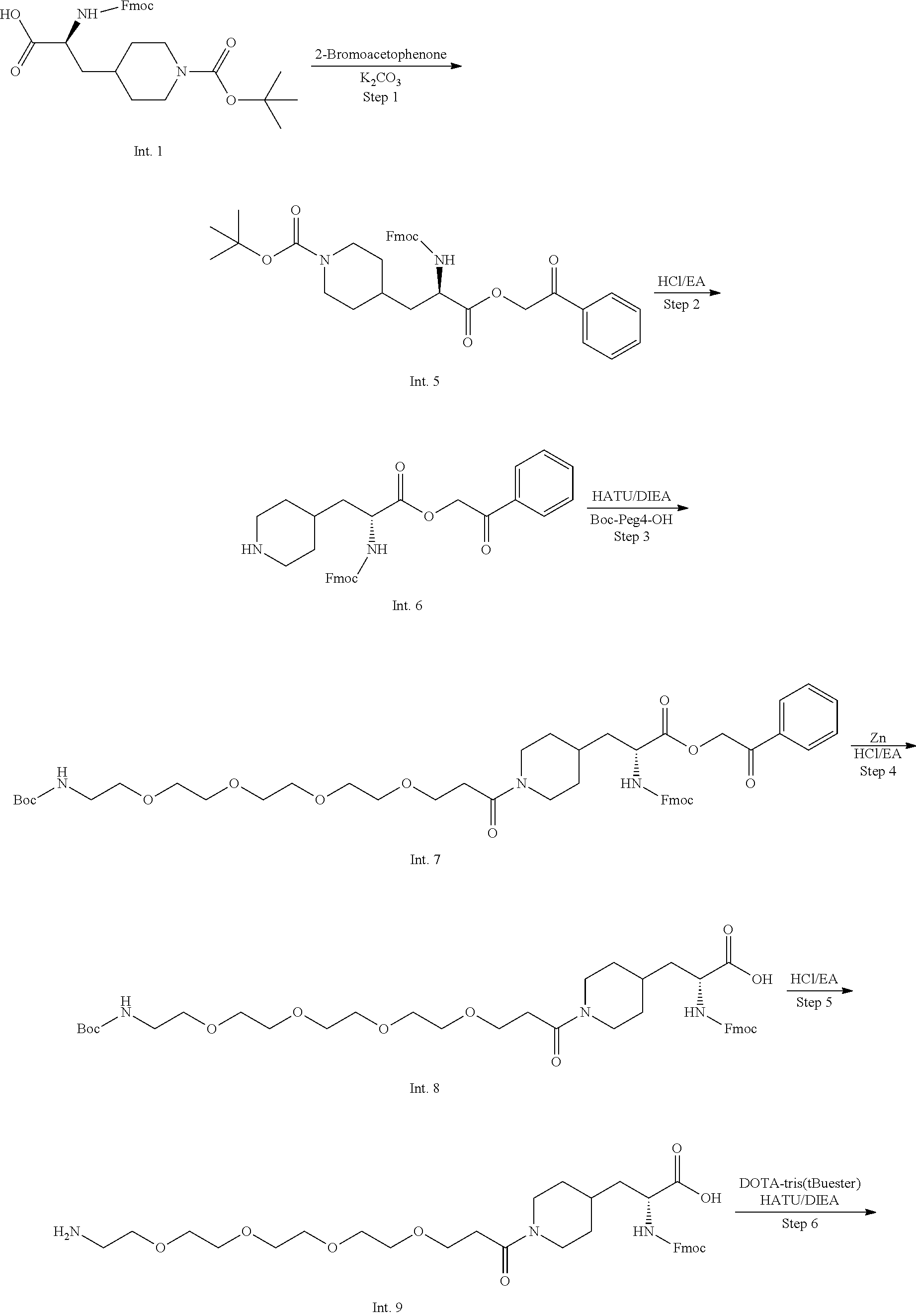

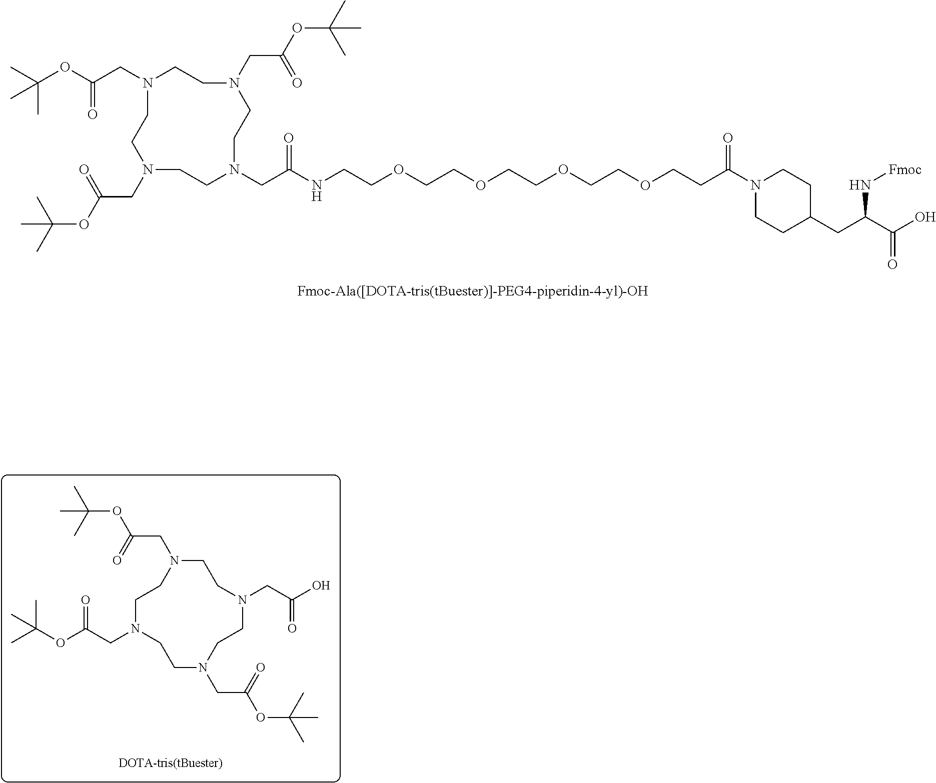

Step 1: To a solution of Int. 1 (5 g, 10.11 mmol) in 50 mL DMF was added K₂CO3 (1.53 g, 11.12 mmol) and 2-Bromoacetophenone (2.2 g, 11.12 mmol) sequentially, the solution was stirred at RT for 1 hour. Upon completion, EA/H₂O (100 mL: 200 mL) was added to the above reaction solution, then the EA phase was separated and washed by sat. NaCl solution once. The EA phase was collected and dried by Na₂SO₄ for 1.5 hours and then EA was evaporated to get the crude Int. 5 (6.0 g) without further purification.

Step 2: To a solution of Int. 5 (6 g) was added 120 mL HCl/EA (4 N) buffer and stirred for 2 hours at RT. The above solution was evaporated directly to get the Int. 6 and was carried over to the next step without further purification.

Step 3: To a solution of Boc-PEG4-OH 3.7 g (10.11 mmol) and HATU 3.84 g (10.11 mmol) in 20 mL DMF at 0-4° C. was added DIEA and stirred for 5-15 minutes. Then, all of the above Int. 6 was dissolved by 30 mL DMF was added dropwise to the solution, and stirred at RT until TLC (DCM:MeOH:AcOH=40:1:0.5) showed the Int. 6 was consumed. Upon completion, EA/H₂O (150 mL: 300 mL) was added to the above reaction solution, then the EA phase was separated and washed by sat. NaCl solution once. The EA phase was collected and dried by Na₂SO₄ for half an hour and then EA was evaporated to get the crude Int. 7 (10 g) and carried over to the next step without further purification.

Step 4: To a solution of Int. 7 in 70 mL AcOH/H₂O (9:1) was added Zn powder 4.0 g (60.66 mmol) activated by conc. HCl, and then stirred at RT overnight. Upon completion, the solution was filtered and the EA filtrate was collected and washed by sat. NaCl solution once. The EA phase was collected and dried by Na₂SO₄ for half an hour and then EA was evaporated to get the crude Int. 8 as oil. Then the Int. 8 was redissolved by 40 mL EA and added a (PE/EA=10:1) solution 500 mL dropwise and stirred overnight to get Int. 8 as a solid (8.0 g).

Step 5: To a solution of Int. 8 (8.0 g) was added 120 mL HCl/EA (4N) buffer and stirred for 2 hours at RT. Upon completion, the above solution was concentrated to dryness to give Int. 9 and carried over to the next step without further purification.

Step 6: To a solution of DOTA-tris(tBuester) 5.73 g (10.0 mmol) and HATU 3.8 g (10.0 mmol) in 20 mL DMF at 0-4° C. was added DIEA and stirred for 5-15 minutes. Then, all of the above Int. 97.6 g dissolved by 30 mL DMF was added dropwise to the solution, and stirred at RT for 2 hours. Additional DOTA-tris(tBuester) 5.73 g (10.0 mmol) was added to the reaction and kept stirring overnight. Upon completion, EA/H₂O (150 mL: 300 mL) was added to the above reaction solution, then 5% H₃PO₄ buffer was added to adjust the pH to 3-4, then the EA phase was separated and washed by sat. NaCl solution once. The EA phase was collected and dried by Na₂SO₄ for half an hour and then EA was evaporated to get the crude Compound 8 and then purified by RP-PHLC to afford the title compound as a solid (4.36 g).

### General Procedure A

### Example Cpd #67

Peptides were synthesized using the CEM Liberty Blue microwave peptide synthesizer. Standard Fmoc chemistry and couplings were used with Rink Amide resin or Wang Resin.

### Synthesis

1. Deprotection: 20% piperidine in DMF (4 mL) was added to the resin and the mixture was heated to 90° C. for 1 minute. The resin was then washed with 4 mL of DMF 4 times.
2. Coupling: To the microwave reaction vessel was added Fmoc-protected amino acid (5 equiv.), DIC (10 equiv.) in DMF (1 M), and OxymaPure (5 equiv.) in DMF (1 M). The microwave reaction vessel is then heated to 90° C. for 2 minutes and then drained. For a double coupling, this process was repeated. For a triple coupling, this process was repeated two times. (Double coupling were primarily used, single couplings were used for precious building blocks, and triple coupling were used for difficult couplings)
3. Iteration: Steps 1 and 2 were repeated for the remaining amino acids.
4. Capping: Deprotection protocol from Step 1 was repeated. 4 mL of 10% Ac2O in DMF was added to the microwave reaction vessel and then heated to 65° C. for 2 minutes. The resin was then washed with 4 mL of DMF 4 times.
5. Orthogonal Deprotection (Dde): The resin is washed with 4 mL of DMF 2 times. To the microwave reaction vessel was added 4 mL of 2% hydrazine in DMF, which was mixed at room temperature for 30 minutes. The resin is then washed with 4 mL of DMF and additional 5 times.
6. DOTA Coupling: To the microwave reaction vessel was added Fmoc-protected amino acid (5 equiv.), DIC (10 equiv.) in DMF (1 M), and OxymaPure (5 equiv.) in DMF (1 M). The microwave reaction vessel is then heated to 90° C. for 4 minutes and then drained. This step is repeated an additional two times.

### Peptide Cleavage, Cyclization and Purification

1. Using the CEM Razor peptide cleavage system, the peptide resin was transferred to a fritted cleavage tube, and 8 mL of cleavage solution (TFA/DODT/H₂O/TIS, 92.5/2.5/2.5/2.5, v/v/v/v) was added to each tube. The peptide resin in cleavage solution was heated to 40° C. for 45 minutes.
2. Each tube is filtered, and the filtrate is collected in centrifuge tubes.
3. The peptide is then precipitated with cold ether (45 mL). The precipitated peptides are then placed on to a centrifuge and allowed to spin for at least 5 minutes. The ether is then decanted from each tube, leaving the peptide at the bottom. This ether precipitation process is then repeated a second time.
4. The peptide pellet is then dissolved in 15 mL of MeCN/H₂O (1/1, v/v), and 0.1 M I₂ in MeOH was added dropwise until a yellow color persisted. The mixture was shaken at room temperature for 10 minutes. The reaction was quenched by adding 0.1 M Na₂S₂O₃ in water, dropwise until the yellow color disappeared. The reaction solution is then concentrated under reduced pressure.
5. Purification: The crude peptide within 2% v/v trifluoroacetic acid (TFA) in 1:1 acetonitrile/water was purified by preparative reversed-phase HPLC under acidic conditions, followed by lyophilization to obtain the product.
   1. Column: XSelect Peptide CSH C18, 1.9 cm i.d.×25 cm (5 μm/130 Å)
2. Mobile phase: Mobile Phase A: 0.05% v/v trifluoroacetic acid in water; Mobile phase B: acetonitrile
3. Gradient: 10% B for 4 minutes; 10→20% B over 4 minutes; 20→30% B over 20 minutes; 30→95% B over 4 minutes; 95% B for 2 minutes. Column temperature: ambient. Flow rate: 24 mL/minute.

### General Procedure B

### Example Cpd #539

Peptides were synthesized using the CEM Liberty Blue microwave peptide synthesizer. Standard Fmoc chemistry and couplings were used with 2-Chloro-Trityl Resin

### Resin Loading

Preloaded 2-Chloro-Trityl Resins were used when available. If the preloaded resin was not available, this procedure was used to manually load the resin with the first amino acid.

To a fritted syringe was added 1.0 g of 2-Chlorotrityl Chloride Resin. The resin was washed with 4 mL of DMF three times, followed by washing with 4 mL of DCM three times to swell the resin. In a separate vial, 12 mL of DCM was added, followed by 2 equiv. DIPEA and 1.2 equiv. of the desired amino acid (1st amino acid in sequence). This solution was added to the resin and allowed to shake for 2 hours. The reaction mixture was then drained. The coupling protocol was repeated a second time. After the second coupling, the resin was washed with 4 mL of DMF three times, followed by 4 mL of DCM three times. To a separate vial was added 17 mL DCM, 2 mL of MeOH, and 1 mL of DIPEA. The solution was added to the resin and allowed to shake for 2 hours. The reaction mixture was then drained and washed with 4 mL of DCM three times, followed by 4 mL of DMF three times. A solution of 8 mL of 20% piperidine in DMF was added to the resin and shaken at room temperature for 15 minutes. The reaction mixture was drained and washed with 4 mL of DMF three times and 4 mL of DCM three times. The resin was then placed under vacuum to dry.

### Synthesis

1. Deprotection: 20% piperidine in DMF (4 mL) was added to the resin and the mixture was heated to 75° C. for 3 minutes. The resin was then washed with 4 mL of DMF 4 times.
2. Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. For a double coupling, this process was repeated. For a triple coupling, this process was repeated two times. (Double coupling were primarily used, single couplings were used for precious building blocks, and triple coupling were used for difficult couplings)
3. Iteration: Steps 1 and 2 were repeated for the remaining amino acids.
4. Capping: Steps 1 and 2 were used with Acetic Acid as the capping reagent.
5. Orthogonal Deprotection (Dde): The resin is washed with 4 mL of DMF 2 times. To the microwave reaction vessel was added 4 mL of 2% hydrazine in DMF, which was mixed at room temperature for 30 minutes. The resin is then washed with 4 mL of DMF and additional 5 times.
6. If there are amino acids between the deprotection in step 5 and DOTA, those amino acids are installed by repeating steps 1 and 2.
7. DOTA Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. This step is repeated an additional two times.

### Peptide Cleavage, Cyclization and Purification

1. Using the CEM Razor peptide cleavage system, the peptide resin was transferred to a fritted cleavage tube, and 8 mL of cleavage solution (TFA/DODT/H2O/TIS, 92.5/2.5/2.5/2.5, v/v/v/v) was added to each tube. The peptide resin in cleavage solution was heated to 40° C. for 45 minutes.
2. Each tube is filtered, and the filtrate is collected in centrifuge tubes.
3. The peptide is then precipitated with cold ether (45 mL). The precipitated peptides are then placed on to a centrifuge and allowed to spin for at least 5 minutes. The ether is then decanted from each tube, leaving the peptide at the bottom. This ether precipitation process is then repeated a second time.
4. The peptide pellet is then dissolved in 15 mL of MeCN/H₂O (1/1, v/v), and 0.1 M I₂ in MeOH was added dropwise until a yellow color persisted. The mixture was shaken at room temperature for 10 minutes. The reaction was quenched by adding 0.1 M Na₂S₂O₃ in water, dropwise until the yellow color disappeared. The reaction solution is then concentrated under reduced pressure.
5. Purification: The crude peptide within 2% v/v trifluoroacetic acid (TFA) in 1:1 acetonitrile/water was purified by preparative reversed-phase HPLC under acidic conditions, followed by lyophilization to obtain the product.
   1. Column: XSelect Peptide CSH C18, 1.9 cm i.d.×25 cm (5 μm/130 Å)
   2. Mobile phase: Mobile Phase A: 0.05% v/v trifluoroacetic acid in water; Mobile phase B: acetonitrile
   3. Gradient: 10% B for 4 minutes; 10→20% B over 4 minutes; 20→30% B over 20 minutes; 30→95% B over 4 minutes; 95% B for 2 minutes. Column temperature: ambient. Flow rate: 24 mL/minute.

### General Procedure C

### Example Cpd #550

Peptides were synthesized using the CEM Liberty Blue microwave peptide synthesizer. Standard Fmoc chemistry and couplings were used with 2-Chloro-Trityl Resin

### Resin Loading

Preloaded 2-Chloro-Trityl Resins were used when available. If the preloaded resin was not available, this procedure was used to manually load the resin with the first amino acid. To a fritted syringe was added 1.0 g of 2-Chlorotrityl Chloride Resin. The resin was washed with 4 mL of DMF three times, followed by washing with 4 mL of DCM three times to swell the resin. In a separate vial, 12 mL of DCM was added, followed by 2 equiv. DIPEA and 1.2 eq of the desired amino acid (1st amino acid in sequence). This solution was added to the resin and allowed to shake for 2 hours. The reaction mixture was then drained. The coupling protocol was repeated a second time. After the second coupling, the resin was washed with 4 mL of DMF three times, followed by 4 mL of DCM three times. To a separate vial was added 17 mL DCM, 2 mL of MeOH, and 1 mL of DIPEA. The solution was added to the resin and allowed to shake for 2 hours. The reaction mixture was then drained and washed with 4 mL of DCM three times, followed by 4 mL of DMF three times. A solution of 8 mL of 20% piperidine in DMF was added to the resin and shaken at room temperature for 15 minutes. The reaction mixture was drained and washed with 4 mL of DMF three times and 4 mL of DCM three times. The resin was then placed under vacuum to dry.

### Synthesis

1. Deprotection: 20% piperidine in DMF (4 mL) was added to the resin and the mixture was heated to 75° C. for 3 minutes. The resin was then washed with 4 mL of DMF 4 times.
2. Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. For a double coupling, this process was repeated. For a triple coupling, this process was repeated two times. (Double coupling were primarily used, single couplings were used for precious building blocks, and triple coupling were used for difficult couplings)
3. Iteration: Steps 1 and 2 were repeated for the remaining amino acids.
4. Capping: Steps 1 and 2 were used with Acetic Acid as the capping reagent.
5. Orthogonal Deprotection (Allyl/Alloc): The resin was washed with 4 mL of DCM three times. In a separate vial, Pd(PPh₃)₄ (0.5 equiv.), PhSiH₃ (5 equiv.), and 6 mL of DCM were added. The solution was added to the resin and shaken at room temperature for 15 minutes. The reaction mixture was drained. This protocol was repeated a second time. The resin was then washed with 4 mL of DMF three times, followed by 4 mL of DCM three times.
6. If there are amino acids between the deprotection in step 5 and DOTA, those amino acids are installed by repeating steps 1 and 2.
7. DOTA Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. This step is repeated an additional two times.

### Peptide Cleavage, Cyclization and Purification

1. Using the CEM Razor peptide cleavage system, the peptide resin was transferred to a fritted cleavage tube, and 8 mL of cleavage solution (TFA/DODT/H2O/TIS, 92.5/2.5/2.5/2.5, v/v/v/v) was added to each tube. The peptide resin in cleavage solution was heated to 40° C. for 45 minutes.
2. Each tube is filtered, and the filtrate is collected in centrifuge tubes.
3. The peptide is then precipitated with cold ether (45 mL). The precipitated peptides are then placed on to a centrifuge and allowed to spin for at least 5 minutes. The ether is then decanted from each tube, leaving the peptide at the bottom. This ether precipitation process is then repeated a second time.
4. The peptide pellet is then dissolved in 15 mL of MeCN/H₂O (1/1, v/v), and 0.1 M I₂ in MeOH was added dropwise until a yellow color persisted. The mixture was shaken at room temperature for 10 minutes. The reaction was quenched by adding 0.1 M Na₂S₂O₃ in water, dropwise until the yellow color disappeared. The reaction solution is then concentrated under reduced pressure.
5. Purification: The crude peptide within 2% v/v trifluoroacetic acid (TFA) in 1:1 acetonitrile/water was purified by preparative reversed-phase HPLC under acidic conditions, followed by lyophilization to obtain the product.
   1. Column: XSelect Peptide CSH C18, 1.9 cm i.d.×25 cm (5 μm/130 Å)
   2. Mobile phase: Mobile Phase A: 0.05% v/v trifluoroacetic acid in water; Mobile phase B: acetonitrile
   3. Gradient: 10% B for 4 minutes; 10→20% B over 4 minutes; 20→30% B over 20 minutes; 30→95% B over 4 minutes; 95% B for 2 minutes. Column temperature: ambient. Flow rate: 24 mL/minute.

### General Procedure C*

### Example Cpd #364

Peptides were synthesized using the CEM Liberty Blue microwave peptide synthesizer. Standard Fmoc chemistry and couplings were used with 2-Chloro-Trityl Resin

### Resin Loading

Preloaded 2-Chloro-Trityl Resins were used when available. If the preloaded resin was not available, this procedure was used to manually load the resin with the first amino acid. To a fritted syringe was added 1.0 g of 2-Chlorotrityl Chloride Resin. The resin was washed with 4 mL of DMF three times, followed by washing with 4 mL of DCM three times to swell the resin. In a separate vial, 12 mL of DCM was added, followed by 2 equiv. DIPEA and 1.2 equiv. of the desired amino acid (1st amino acid in sequence). This solution was added to the resin and allowed to shake for 2 hours. The reaction mixture was then drained. The coupling protocol was repeated a second time. After the second coupling, the resin was washed with 4 mL of DMF three times, followed by 4 mL of DCM three times. To a separate vial was added 17 mL DCM, 2 mL of MeOH, and 1 mL of DIPEA. The solution was added to the resin and allowed to shake for 2 hours. The reaction mixture was then drained and washed with 4 mL of DCM three times, followed by 4 mL of DMF three times. A solution of 8 mL of 20% piperidine in DMF was added to the resin and shaken at room temperature for 15 minutes. The reaction mixture was drained and washed with 4 mL of DMF three times and 4 mL of DCM three times. The resin was then placed under vacuum to dry.

### Synthesis

1. Deprotection: 20% piperidine in DMF (4 mL) was added to the resin and the mixture was heated to 75° C. for 3 minutes. The resin was then washed with 4 mL of DMF 4 times.
2. Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. For a double coupling, this process was repeated. For a triple coupling, this process was repeated two times. (Double coupling were primarily used, single couplings were used for precious building blocks, and triple coupling were used for difficult couplings)
3. Iteration: Steps 1 and 2 were repeated for the remaining amino acids.
4. Capping: Steps 1 and 2 were used with Acetic Acid as the capping reagent.
5. Orthogonal Deprotection (Allyl/Alloc): The resin was washed with 4 mL of DCM three times. In a separate vial, Pd(PPh₃)₄ (0.5 equiv.), PhSiH₃ (5 equiv.), and 6 mL of DCM were added. The solution was added to the resin and shaken at room temperature for 15 minutes. The reaction mixture was drained. This protocol was repeated a second time. The resin was then washed with 4 mL of DMF three times, followed by 4 mL of DCM three times.
6. Steps 1 and 2 were repeated for the amino acids coming off the deprotection in step 5.
7. Orthogonal Deprotection (Dde): The resin is washed with 4 mL of DMF 2 times. To the microwave reaction vessel was added 4 mL of 2% hydrazine in DMF, which was mixed at room temperature for 30 minutes. The resin is then washed with 4 mL of DMF and additional 5 times.
8. If there are amino acids between the deprotection in step 7 and DOTA, those amino acids are installed by repeating steps 1 and 2.
9. DOTA Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. This step is repeated an additional two times.

### Peptide Cleavage, Cyclization and Purification

1. Using the CEM Razor peptide cleavage system, the peptide resin was transferred to a fritted cleavage tube, and 8 mL of cleavage solution (TFA/DODT/H2O/TIS, 92.5/2.5/2.5/2.5, v/v/v/v) was added to each tube. The peptide resin in cleavage solution was heated to 40° C. for 45 minutes.
2. Each tube is filtered, and the filtrate is collected in centrifuge tubes.
3. The peptide is then precipitated with cold ether (45 mL). The precipitated peptides are then placed on to a centrifuge and allowed to spin for at least 5 minutes. The ether is then decanted from each tube, leaving the peptide at the bottom. This ether precipitation process is then repeated a second time.
4. The peptide pellet is then dissolved in 15 mL of MeCN/H₂O (1/1, v/v), and 0.1 M I₂ in MeOH was added dropwise until a yellow color persisted. The mixture was shaken at room temperature for 10 minutes. The reaction was quenched by adding 0.1 M Na₂S₂O₃ in water, dropwise until the yellow color disappeared. The reaction solution is then concentrated under reduced pressure.
5. Purification: The crude peptide within 2% v/v trifluoroacetic acid (TFA) in 1:1 acetonitrile/water was purified by preparative reversed-phase HPLC under acidic conditions, followed by lyophilization to obtain the product.
   1. Column: XSelect Peptide CSH C18, 1.9 cm i.d.×25 cm (5 μm/130 Å)
   2. Mobile phase: Mobile Phase A: 0.05% v/v trifluoroacetic acid in water; Mobile phase B: acetonitrile 3. Gradient: 10% B for 4 minutes; 10→20% B over 4 minutes; 20→30% B over 20 minutes; 30→95% B over 4 minutes; 95% B for 2 minutes. Column temperature: ambient. Flow rate: 24 mL/minute.

### General Procedure D

### Example Cpd #5

Peptides were synthesized using the CEM Liberty Blue microwave peptide synthesizer. Standard Fmoc chemistry and couplings were used with Rink Amide resin or Wang Resin.

### Synthesis

1. Deprotection: 20% piperidine in DMF (4 mL) was added to the resin and the mixture was heated to 90° C. for 1 minute. The resin was then washed with 4 mL of DMF 4 times.
2. Coupling: To the microwave reaction vessel was added Fmoc-protected amino acid (5 equiv.), DIC (10 equiv.) in DMF (1 M), and OxymaPure (5 equiv.) in DMF (1 M). The microwave reaction vessel is then heated to 90° C. for 2 minutes and then drained. For a double coupling, this process was repeated. For a triple coupling, this process was repeated two times. (Double coupling were primarily used, single couplings were used for precious building blocks, and triple coupling were used for difficult couplings)
3. Iteration: Steps 1 and 2 were repeated for the remaining amino acids.
4. N-Terminus DOTA Coupling: To the microwave reaction vessel was added Fmoc-protected amino acid (5 equiv.), DIC (10 equiv.) in DMF (1 M), and OxymaPure (5 equiv.) in DMF (1 M). The microwave reaction vessel is then heated to 90° C. for 4 minutes and then drained. This step is repeated an additional two times.

### Peptide Cleavage, Cyclization and Purification

1. Using the CEM Razor peptide cleavage system, the peptide resin was transferred to a fritted cleavage tube, and 8 mL of cleavage solution (TFA/DODT/H2O/TIS, 92.5/2.5/2.5/2.5, v/v/v/v) was added to each tube. The peptide resin in cleavage solution was heated to 40° C. for 45 minutes.
2. Each tube is filtered, and the filtrate is collected in centrifuge tubes.
3. The peptide is then precipitated with cold ether (45 mL). The precipitated peptides are then placed on to a centrifuge and allowed to spin for at least 5 minutes. The ether is then decanted from each tube, leaving the peptide at the bottom. This ether precipitation process is then repeated a second time.
4. The peptide pellet is then dissolved in 15 mL of MeCN/H₂O (1/1, v/v), and 0.1 M I₂ in MeOH was added dropwise until a yellow color persisted. The mixture was shaken at room temperature for 10 minutes. The reaction was quenched by adding 0.1 M Na₂S₂O₃ in water, dropwise until the yellow color disappeared. The reaction solution is then concentrated under reduced pressure.
5. Purification: The crude peptide within 2% v/v trifluoroacetic acid (TFA) in 1:1 acetonitrile/water was purified by preparative reversed-phase HPLC under acidic conditions, followed by lyophilization to obtain the product.
   1. Column: XSelect Peptide CSH C18, 1.9 cm i.d.×25 cm (5 μm/130 Å)
   2. Mobile phase: Mobile Phase A: 0.05% v/v trifluoroacetic acid in water; Mobile phase B: acetonitrile
   3. Gradient: 10% B for 4 minutes; 10→20% B over 4 minutes; 20→30% B over 20 minutes; 30→95% B over 4 minutes; 95% B for 2 minutes. Column temperature: ambient. Flow rate: 24 mL/minute.

### General Procedure E

### Example Cpd #306

Peptides were synthesized using the CEM Liberty Blue microwave peptide synthesizer. Standard Fmoc chemistry and couplings were used with 2-Chloro-Trityl Resin

### Resin Loading

Preloaded 2-Chloro-Trityl Resins were used when available. If the preloaded resin was not available, this procedure was used to manually load the resin with the first amino acid. To a fritted syringe was added 1.0 g of 2-Chlorotrityl Chloride Resin. The resin was washed with 4 mL of DMF three times, followed by washing with 4 mL of DCM three times to swell the resin. In a separate vial, 12 mL of DCM was added, followed by 2 equiv. DIPEA and 1.2 equiv. of the desired amino acid (1st amino acid in sequence). This solution was added to the resin and allowed to shake for 2 hours. The reaction mixture was then drained. The coupling protocol was repeated a second time. After the second coupling, the resin was washed with 4 mL of DMF three times, followed by 4 mL of DCM three times. To a separate vial was added 17 mL DCM, 2 mL of MeOH, and 1 mL of DIPEA. The solution was added to the resin and allowed to shake for 2 hours. The reaction mixture was then drained and washed with 4 mL of DCM three times, followed by 4 mL of DMF three times. A solution of 8 mL of 20% piperidine in DMF was added to the resin and shaken at room temperature for 15 minutes. The reaction mixture was drained and washed with 4 mL of DMF three times and 4 mL of DCM three times. The resin was then placed under vacuum to dry.

### Synthesis

1. Deprotection: 20% piperidine in DMF (4 mL) was added to the resin and the mixture was heated to 75° C. for 3 minutes. The resin was then washed with 4 mL of DMF 4 times.
2. Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. For a double coupling, this process was repeated. For a triple coupling, this process was repeated two times. (Double coupling were primarily used, single couplings were used for precious building blocks, and triple coupling were used for difficult couplings)
3. Iteration: Steps 1 and 2 were repeated for the remaining amino acids.
4. N-Terminus DOTA Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. This step is repeated an additional two times.

### Peptide Cleavage, Cyclization and Purification

1. Using the CEM Razor peptide cleavage system, the peptide resin was transferred to a fritted cleavage tube, and 8 mL of cleavage solution (TFA/DODT/H2O/TIS, 92.5/2.5/2.5/2.5, v/v/v/v) was added to each tube. The peptide resin in cleavage solution was heated to 40° C. for 45 minutes.
2. Each tube is filtered, and the filtrate is collected in centrifuge tubes.
3. The peptide is then precipitated with cold ether (45 mL). The precipitated peptides are then placed on to a centrifuge and allowed to spin for at least 5 minutes. The ether is then decanted from each tube, leaving the peptide at the bottom. This ether precipitation process is then repeated a second time.
4. The peptide pellet is then dissolved in 15 mL of MeCN/H₂O (1/1, v/v), and 0.1 M I₂ in MeOH was added dropwise until a yellow color persisted. The mixture was shaken at room temperature for 10 minutes. The reaction was quenched by adding 0.1 M Na₂S₂O₃ in water, dropwise until the yellow color disappeared. The reaction solution is then concentrated under reduced pressure.
5. Purification: The crude peptide within 2% v/v trifluoroacetic acid (TFA) in 1:1 acetonitrile/water was purified by preparative reversed-phase HPLC under acidic conditions, followed by lyophilization to obtain the product.
   1. Column: XSelect Peptide CSH C18, 1.9 cm i.d.×25 cm (5 μm/130 Å)
   2. Mobile phase: Mobile Phase A: 0.05% v/v trifluoroacetic acid in water; Mobile phase B: acetonitrile
   3. Gradient: 10% B for 4 minutes; 10→20% B over 4 minutes; 20→30% B over 20 minutes; 30→95% B over 4 minutes; 95% B for 2 minutes. Column temperature: ambient. Flow rate: 24 mL/minute.

### General Procedure G

### Example Cpd #363

Peptides were synthesized using the CEM Liberty Blue microwave peptide synthesizer. Standard Fmoc chemistry and couplings were used with 2-Chloro-Trityl Resin

### Resin Loading

To a fritted syringe was added 1.0 g of 2-Chlorotrityl Chloride Resin. The resin was washed with 4 mL of DMF three times, followed by washing with 4 mL of DCM three times to swell the resin. In a separate vial, 12 mL of DCM was added, followed by 2 equiv. DIPEA and 1.2 equiv. of the desired amino acid (1st amino acid in sequence). This solution was added to the resin and allowed to shake for 2 hours. The reaction mixture was then drained. The coupling protocol was repeated a second time. After the second coupling, the resin was washed with 4 mL of DMF three times, followed by 4 mL of DCM three times. To a separate vial was added 17 mL DCM, 2 mL of MeOH, and 1 mL of DIPEA. The solution was added to the resin and allowed to shake for 2 hours. The reaction mixture was then drained and washed with 4 mL of DCM three times, followed by 4 mL of DMF three times. A solution of 8 mL of 20% piperidine in DMF was added to the resin and shaken at room temperature for 15 minutes. The reaction mixture was drained and washed with 4 mL of DMF three times and 4 mL of DCM three times. The resin was then placed under vacuum to dry.

This procedure specifically used Dde-d-Lys(Fmoc)-OH to load the resin.

### Synthesis

1. Deprotection: 20% piperidine in DMF (4 mL) was added to the resin and the mixture was heated to 75° C. for 3 minutes. The resin was then washed with 4 mL of DMF 4 times.
2. DOTA Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. This step is repeated an additional two times.
3. Orthogonal Deprotection (Dde): The resin is washed with 4 mL of DMF 2 times. To the microwave reaction vessel was added 4 mL of 2% hydrazine in DMF, which was mixed at room temperature for 30 minutes. The resin is then washed with 4 mL of DMF and additional 5 times.
4. Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. For a double coupling, this process was repeated. For a triple coupling, this process was repeated two times. (Double coupling were primarily used, single couplings were used for precious building blocks, and triple coupling were used for difficult couplings)
5. Steps 1 and 4 were repeated for the remaining amino acids.
6. Capping: Steps 1 and 4 were used with Acetic Acid as the capping reagent.
7. Step 3 was repeated to remove the 2^{nd} Dde group that was installed in step 5.
8. Steps 1 and 4 were repeated to add the amino acids coming off the 2^{nd} Dde orthogonal deprotection.

### Peptide Cleavage, Cyclization and Purification

1. Using the CEM Razor peptide cleavage system, the peptide resin was transferred to a fritted cleavage tube, and 8 mL of cleavage solution (TFA/DODT/H2O/TIS, 92.5/2.5/2.5/2.5, v/v/v/v) was added to each tube. The peptide resin in cleavage solution was heated to 40° C. for 45 minutes.
2. Each tube is filtered, and the filtrate is collected in centrifuge tubes.
3. The peptide is then precipitated with cold ether (45 mL). The precipitated peptides are then placed on to a centrifuge and allowed to spin for at least 5 minutes. The ether is then decanted from each tube, leaving the peptide at the bottom. This ether precipitation process is then repeated a second time.
4. The peptide pellet is then dissolved in 15 mL of MeCN/H₂O (1/1, v/v), and 0.1 M I₂ in MeOH was added dropwise until a yellow color persisted. The mixture was shaken at room temperature for 10 minutes. The reaction was quenched by adding 0.1 M Na₂S₂O₃ in water, dropwise until the yellow color disappeared. The reaction solution is then concentrated under reduced pressure.
5. Purification: The crude peptide within 2% v/v trifluoroacetic acid (TFA) in 1:1 acetonitrile/water was purified by preparative reversed-phase HPLC under acidic conditions, followed by lyophilization to obtain the product.
   1. Column: XSelect Peptide CSH C18, 1.9 cm i.d.×25 cm (5 μm/130 Å)
   2. Mobile phase: Mobile Phase A: 0.05% v/v trifluoroacetic acid in water; Mobile phase B: acetonitrile
   3. Gradient: 10% B for 4 minutes; 10→20% B over 4 minutes; 20→30% B over 20 minutes; 30→95% B over 4 minutes; 95% B for 2 minutes. Column temperature: ambient. Flow rate: 24 mL/minute.

### General Procedure I

### Example Cpd #482

Peptides were synthesized using the CEM Liberty Blue microwave peptide synthesizer. Standard Fmoc chemistry and couplings were used with 2-Chloro-Trityl Resin

### Resin Loading

To a fritted syringe was added 1.0 g of 2-Chlorotrityl Chloride Resin. The resin was washed with 4 mL of DMF three times, followed by washing with 4 mL of DCM three times to swell the resin. In a separate vial, 12 mL of DCM was added, followed by 2 equiv. DIPEA and 1.2 equiv. of the desired amino acid (1st amino acid in sequence). This solution was added to the resin and allowed to shake for 2 hours. The reaction mixture was then drained. The coupling protocol was repeated a second time. After the second coupling, the resin was washed with 4 mL of DMF three times, followed by 4 mL of DCM three times. To a separate vial was added 17 mL DCM, 2 mL of MeOH, and 1 mL of DIPEA. The solution was added to the resin and allowed to shake for 2 hours. The reaction mixture was then drained and washed with 4 mL of DCM three times, followed by 4 mL of DMF three times. A solution of 8 mL of 20% piperidine in DMF was added to the resin and shaken at room temperature for 15 minutes. The reaction mixture was drained and washed with 4 mL of DMF three times and 4 mL of DCM three times. The resin was then placed under vacuum to dry.

This procedure specifically used Fmoc-Lys(Dde)-OH to load the resin.

### Synthesis

1. Deprotection: 20% piperidine in DMF (4 mL) was added to the resin and the mixture was heated to 75° C. for 3 minutes. The resin was then washed with 4 mL of DMF 4 times.
2. Coupling: To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. For a double coupling, this process was repeated. For a triple coupling, this process was repeated two times. (Double coupling were primarily used, single couplings were used for precious building blocks, and triple coupling were used for difficult couplings)
3. Iteration: Steps 1 and 2 were repeated to add on Val-Met-Ambz.
4. DOTA Coupling: Step 1 was repeated. To the microwave reaction vessel was added Fmoc-protected amino acid (5 equiv.), DIPEA (10 equiv.) in DMF (1 M), and HATU (5 equiv.) in DMF (0.5 M). The microwave reaction vessel is then heated to 75° C. for 5 minutes and then drained. This step is repeated an additional two times.
5. Orthogonal Deprotection (Dde): The resin is washed with 4 mL of DMF 2 times. To the microwave reaction vessel was added 4 mL of 2% hydrazine in DMF, which was mixed at room temperature for 30 minutes. The resin is then washed with 4 mL of DMF and additional 5 times.
6. Steps 1 and 2 were repeated for the remaining amino acids.
7. Capping: Steps 1 and 2 were used with Acetic Acid as the capping reagent.

### Peptide Cleavage, Cyclization and Purification

1. Using the CEM Razor peptide cleavage system, the peptide resin was transferred to a fritted cleavage tube, and 8 mL of cleavage solution (TFA/DODT/H2O/TIS, 92.5/2.5/2.5/2.5, v/v/v/v) was added to each tube. The peptide resin in cleavage solution was heated to 40° C. for 45 minutes.
2. Each tube is filtered, and the filtrate is collected in centrifuge tubes.
3. The peptide is then precipitated with cold ether (45 mL). The precipitated peptides are then placed on to a centrifuge and allowed to spin for at least 5 minutes. The ether is then decanted from each tube, leaving the peptide at the bottom. This ether precipitation process is then repeated a second time.
4. The peptide pellet is then dissolved in 15 mL of MeCN/H₂O (1/1, v/v), and 0.1 M I₂ in MeOH was added dropwise until a yellow color persisted. The mixture was shaken at room temperature for 10 minutes. The reaction was quenched by adding 0.1 M Na₂S₂O₃ in water, dropwise until the yellow color disappeared. The reaction solution is then concentrated under reduced pressure.
5. Purification: The crude peptide within 2% v/v trifluoroacetic acid (TFA) in 1:1 acetonitrile/water was purified by preparative reversed-phase HPLC under acidic conditions, followed by lyophilization to obtain the product.
   1. Column: XSelect Peptide CSH C18, 1.9 cm i.d.×25 cm (5 μm/130 Å)
   2. Mobile phase: Mobile Phase A: 0.05% v/v trifluoroacetic acid in water; Mobile phase B: acetonitrile
   3. Gradient: 10% B for 4 minutes; 10→20% B over 4 minutes; 20→30% B over 20 minutes; 30→95% B over 4 minutes; 95% B for 2 minutes. Column temperature: ambient. Flow rate: 24 mL/minute.

### General Procedure J

### Example Cpd #7

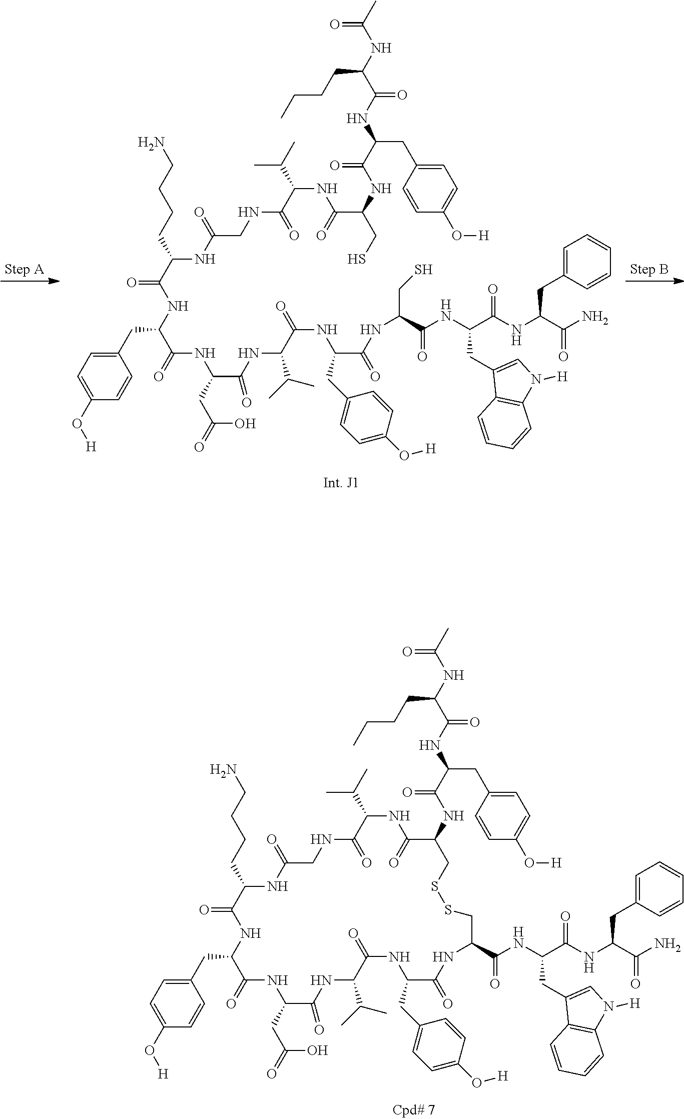

### Step A. Synthesis of Compound Int. J1

The peptide was synthesized by standard Solid-phase Peptide Synthesis (SPPS) using Fmoc/t-Bu chemistry. The assembly was performed on a PS Rink-amide MBHA resin (200 umol, 100-200 Mesh; loading 0.42 mmol/g) on a Liberty Prime microwave peptide synthesizer (CEM Inc.).

All the amino acids were dissolved at a 0.5 M concentration in DMF. The amino acids were activated with equimolar amounts of a 2 M solution of DIC in DMF and a 0.25 M solution of Oxyma in DMF The acylation reactions were performed for 2 minutes at 90° C. under MW irradiation with a 4-fold excess of activated amino acids over the resin free amino groups. The Fmoc deprotection reactions were performed for 3 minutes at 90° C. using a solution of 20% piperidine in DMF.

Double acylation reactions were performed for bulky residues (including alpha-methylated, N-methylated or beta-branched amino acids) and following amino acid residue after the bulky one.

N-terminal acetylation was performed for 2 minutes at 65° C. under MW irradiation with a 10% v/v solution of AC₂O in DMF.

At the end of the assembly the resin was washed with DMF, DCM, Et₂O. 100 umol of the resin bound peptide was cleaved from solid support using 15 mL of TFA solution (v/v: 87.5% TFA, 5% H₂O, 2.5% TIPS, 5% Phenol) for approximately 1.5 hours, at room temperature. The resin was then filtered and concentrated to about 5 mL and precipitated in cold MTBE (40 mL). After centrifugation, the peptide pellets were washed with fresh cold Et₂O to remove the organic scavengers. The process was repeated twice. Final pellets were dried, re-suspended in H₂O and ACN 1:1, stirred overnight and then lyophilized to afford crude Compound Int. J1.

### Step B. Synthesis of Cpd #7

Intermediate Compound J1 was dissolved in a mixture of ACN/H₂O (1:1) at 0.5 mg/ml concentration. Iodine, saturated solution in acetic acid, was added dropwise until the solution becomes persistent yellow. The reaction was stirred for 1.5 hours and quenched with ascorbic acid (powder) until the solution becomes clear again. The reaction mixture was lyophilized to dryness and the crude peptide was dissolved in 3 mL DMSO and purified by reverse-phase HPLC using a preparative Xbridge Waters C18 column (250×50 mm, 130 Å, 5 μm) and the mobile phase A: H₂O+0.1% TFA, and mobile phase B: ACN+0.1% TFA. The following gradient of eluent B was used: 25% B to 25% B over 5 minute, to 45% B over 20 minutes, with a flow rate 80 mL/minute, and UV detection at a wavelength 214 nm. Collected fractions were lyophilized to afford Cpd #7.

### General Procedure K

### Example Cpd #27

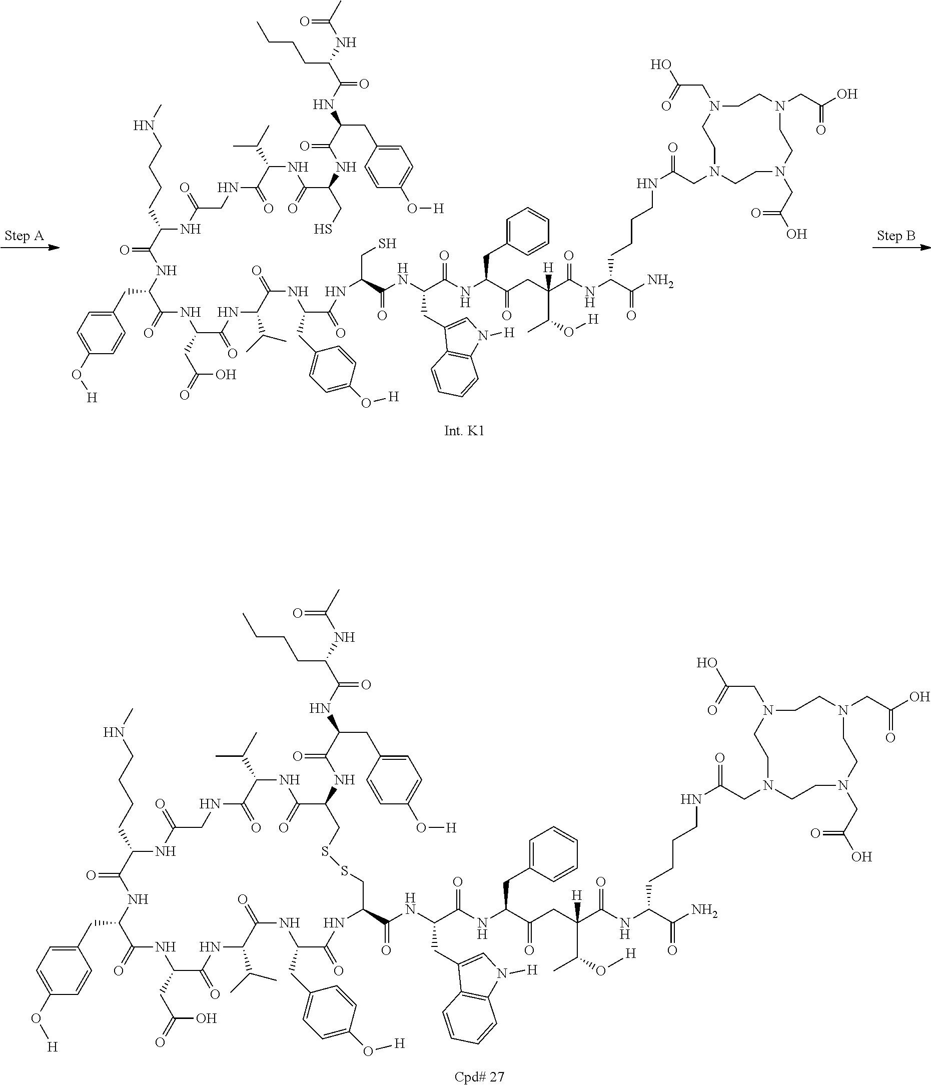

### Step A. Synthesis of Compound Int. K1

The peptide was synthesized by standard Solid-phase Peptide Synthesis (SPPS) using Fmoc/t-Bu chemistry. The assembly was performed on a Rink-amide MBHA resin (100 umol, 100-200 Mesh; loading 0.42 mmol/g) on the Cem Liberty Blue microwave peptide synthesizer (CEM Inc.). During peptide assembly on solid phase, the side chain for D-Lys was protected with Dde.

All the amino acids were dissolved at a 0.2 M concentration in DMF. The acylation reactions were performed for 2 minutes at 90° C. under MW irradiation with 4 folds excess of activated amino acids over the resin free amino groups. The amino acids were activated with equimolar amounts of 1 M solution of DIC in DMF and Oxyma solution 1 M in DMF.

Double acylation reactions were performed for bulky residues (including alpha-methylated, N-methylated or beta-branched amino acids) and following amino acid residue after the bulky one.

N-terminal acetylation was performed for 2 minutes at 65° C. under MW irradiation with a 10% v/v solution of Ac₂O in DMF.

At the end of the assembly, a 3% hydrazine monohydrate solution in DMF (30 mL) was percolated on the resin over 15 minutes to selectively remove the Dde protecting group from the D-Lys.

DOTA was incorporated manually using an equimolar solution of DOTA(tBu)3, DIC and HOBt (3 equiv., 1:1:1) in NMP at room temperature and complete acylation was monitored by ninhydrin test.

At the end of the assembly the resin was washed with DMF, DCM, Et₂O. The peptide was cleaved from solid support using 20 ml of TFA solution (v/v: 87.5% TFA, 5% H2O, 2.5% TIPS, 5% Phenol) for approximately 4 hours, at room temperature. The resin was then filtered and concentrated to about 10 mL and precipitated in cold MTBE (90 mL). After centrifugation, the peptide pellets were washed with fresh cold Et₂O to remove the organic scavengers. The process was repeated twice. Final pellets were dried, re-suspended in H₂O and ACN 1:1 and stirred overnight. Then lyophilized to afford crude Compound Int. K1.

### Step B. Synthesis of Compound Cpd #27

Intermediate Compound Int. K1 was dissolved in H2O/ACN (1 mg/mL). Iodine (saturated solution in AcOH) was added until yellow color persisted. Stirred at room temperature for 5 minutes, then quenched with ascorbic acid and lyophilized.

Crude peptide was dissolved in 1 mL DMSO and purified by reverse-phase HPLC using preparative Waters XBridge C18 column (150×50 mm, 130 Å, 5 μm). Mobile phase A: H2O+0.1% TFA, mobile phase B: ACN+0.1% TFA. The following gradient of eluent B was used: 27% to 42% over 20 minutes, flow rate 80 mL/minute, wavelength 214 nm. The resulting fractions were lyophilized to afford the desired product Cpd #27.

### General Procedure K2

### Synthesis of Compound Fmoc-MAF(Dde)-OH

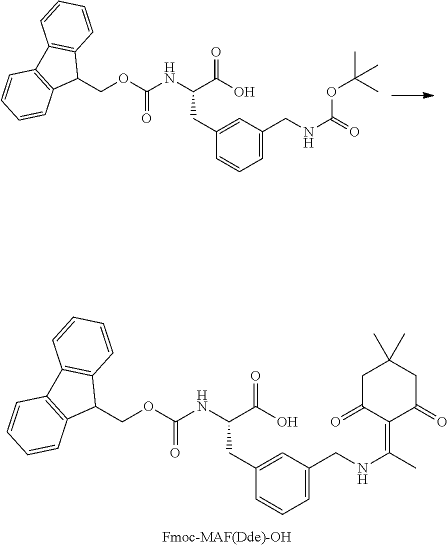 [SMILES: CC(C)(C)OC(=O)NCc1cccc(C[C@H](NC(=O)OCC2c3ccccc3-c3ccccc32)C(=O)O)c1 ; CC(NCc1cccc(C[C@H](NC(=O)OCC2c3ccccc3-c3ccccc32)C(=O)O)c1)=C1C(=O)CC(C)(C)CC1=O]

Fmoc-MAF(Boc)-OH (2.71 g, 1 equiv.) was stirred in 30 mL of a mixture of TFA solution (v/v: 95% TFA, 5% H₂O) for approximately 20 minutes, at room temperature. Then, the solution was concentrated to dryness. The crude material was dissolved in 60 mL of EtOH and DIPEA (8 equiv.) was added. A solution of Dde-OH (1.1 equiv.) dissolved in EtOH (10 mL) was added. Stirred overnight at 50° C.

Reaction crude was concentrated to dryness, re-dissolved in EtOAc and washed twice with HCl 1N and brine. The organic layer was dried on Na₂SO₄, filtered and concentrated to dryness to afford 5.2 g of crude.

Crude material was purified by direct-phase flash chromatography using a Luknova 120 g column and mobile phase A: DCM, mobile phase B: MeOH. The following gradient of eluent B was used: 0% B to 0% B over 3CV, to 10% B over 15CV, flow rate 85 mL/minute, with UV detection at a wavelength 254 nm. Collected fractions were concentrated to dryness to afford Compound Fmoc-MAF(Dde)-OH. LCMS anal. calc. For C₃₅H₃₆N₂O₆: 580.68; found: 581.3 (M+1)⁺

The synthesis of Fmoc-AEF(Dde)-OH follows the same procedure as Fmoc-MAF(Dde)-OH.

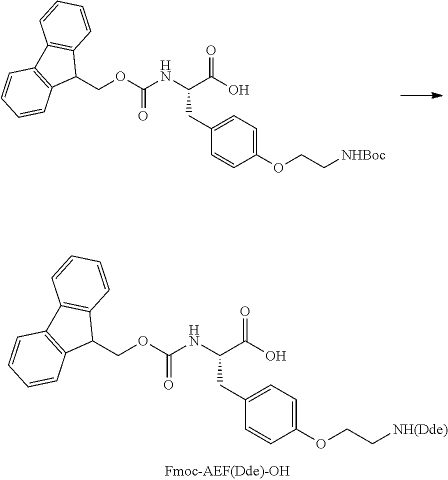

The peptide was synthesized by standard Solid-phase Peptide Synthesis (SPPS) using Fmoc/t-Bu chemistry. The assembly was performed on a Rink-amide MBHA resin (100 umol, 100-200 Mesh; loading 0.42 mmol/g) on the Cem Liberty Blue microwave peptide synthesizer (CEM Inc.). During peptide assembly on solid phase, the side chain for MAF or AEF was protected with Dde.

All the amino acids were dissolved at a 0.2 M concentration in DMF. The acylation reactions were performed for 2 minutes at 90° C. under MW irradiation with 4 folds excess of activated amino acids over the resin free amino groups. The amino acids were activated with equimolar amounts of 1 M solution of DIC in DMF and Oxyma solution 1 M in DMF.

Double acylation reactions were performed for bulky residues (including alpha-methylated, N-methylated or beta-branched amino acids) and following amino acid residue after the bulky one.

N-terminal acetylation was performed for 2 minutes at 65° C. under MW irradiation with a 10% v/v solution of Ac₂O in DMF.

At the end of the assembly, a 3% hydrazine monohydrate solution in DMF (30 mL) was percolated on the resin over 15 minutes to selectively remove the Dde protecting group from MAF or AEF.

DOTA was incorporated manually using an equimolar solution of DOTA(tBu)3, DIC and HOBt (3 equiv., 1:1:1) in NMP at room temperature and complete acylation was monitored by ninhydrin test.

At the end of the assembly the resin was washed with DMF, DCM, Et₂O. The peptide was cleaved from solid support using 20 mL of TFA solution (v/v: 87.5% TFA, 5% H₂O, 2.5% TIPS, 5% Phenol) for approximately 4 hours, at room temperature. The resin was then filtered and concentrated to about 10 mL and precipitated in cold MTBE (90 mL). After centrifugation, the peptide pellets were washed with fresh cold Et₂O to remove the organic scavengers. The process was repeated twice. Final pellets were dried, re-suspended in H₂O and ACN 1:1 and stirred overnight. Then lyophilized to afford crude linear peptide.

The above intermediate linear peptide was dissolved in H₂O/ACN (1:1, 1 mg/ml). Iodine (saturated solution in AcOH) was added until yellow color persisted. Stirred at room temperature for 5 minutes, then quenched with ascorbic acid and lyophilized.

Crude peptide was dissolved in 1 mL DMSO and purified by reverse-phase HPLC using preparative Waters XBridge C18 column (150×50 mm, 130 Å, 5 μm). Mobile phase A: H₂O+0.1% TFA, mobile phase B: ACN+0.1% TFA. The following gradient of eluent B was used: 25% for 5 minutes and then increasing to 45% over 20 minutes, flow rate 80 mL/minute, wavelength 214 nm. The resulting fractions were lyophilized to afford the desired product.

### General Procedure L

### Example Cpd #130

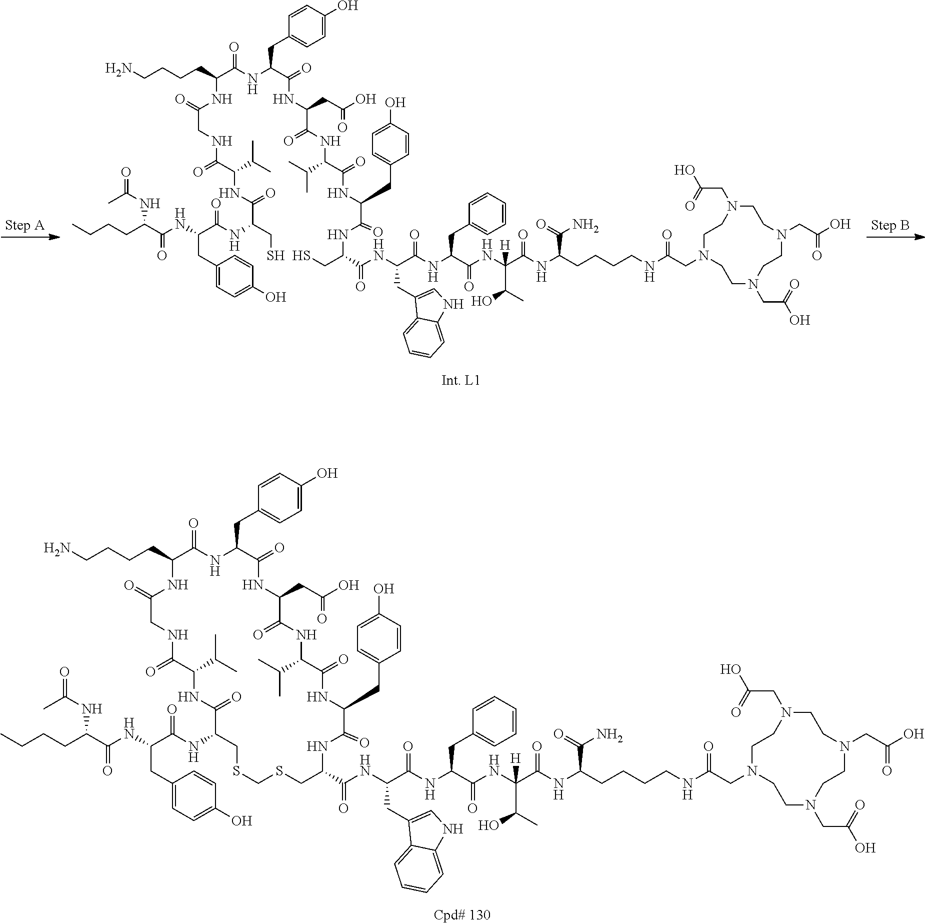

### Step A. Synthesis of Compound Int. L1

The peptide was synthesized by standard Solid-phase Peptide Synthesis (SPPS) using Fmoc/t-Bu chemistry. The assembly was performed on a PS Rink-amide MBHA resin (100 umol, 100-200 Mesh; loading 0.42 mmol/g) on the Cem Liberty Blue microwave peptide synthesizer (CEM In.c.). During peptide assembly on solid phase, the side chain of D-Lys was protected with Dde.

All the amino acids were dissolved at a 0.2 M concentration in DMF. The amino acids were activated with equimolar amounts of a 1 M solution of DIC in DMF and a 1 M solution of Oxyma in DMF. The acylation reactions were performed for 2 minutes at 90° C. under MW irradiation with a 5-fold excess of activated amino acids over the resin free amino groups. The Fmoc deprotection reactions were performed for 3 minutes at 90° C. using a 20% piperidine solution in DMF.

Double acylation reactions were performed for bulky residues (including alpha-methylated, N-methylated or beta-branched amino acids) and following amino acid residue after the bulky one.

N-terminal acetylation was performed for 2 minutes at 65° C. under MW irradiation with a 10% v/v solution of AC₂O in DMF.

Dde protecting group was removed from D-Lys side chain by treating the resin with a 4% solution of hydrazine monohydrate in DMF (20 mL) over 30 minutes at room temperature. DOTA was incorporated by manual coupling using an equimolar mixture of DOTA(tBu)₃, DIC and HOBt (2 equiv.) in NMP, overnight at room temperature.

At the end of the assembly the resin was washed with DMF, DCM, Et₂O. The resin bound peptide was cleaved from solid support using 15 mL of TFA solution (v/v: 87.5% TFA, 5% H₂O, 2.5% TIPS, 5% Phenol) for approximately 4 hours, at room temperature. The resin was then filtered and concentrated to about 5 mL and precipitated in cold MTBE (40 mL). After centrifugation, the peptide pellets were washed with fresh cold Et₂O to remove the organic scavengers. The process was repeated twice. Final pellets were dried, re-suspended in H₂O and ACN 1:1 and stirred overnight. Then lyophilized to afford crude Compound Int. L¹.

### Step B. Synthesis of Compound Cpd #130

Intermediate Compound Int. L¹ was dissolved in a mixture of H2O/ACN (1 mg/mL). TCEP·HCl (3 equiv.) was added, followed by diiodomethane (10 equiv.) and DIPEA (1% v/v). The reaction was stirred at room temperature overnight, then quenched with TFA (5% in water) to adjust pH around 1 and lyophilized. Crude peptide was purified by reverse-phase HPLC using a preparative Waters XBridge C18 column (250×50 mm, 130 Å, 5 μm) with mobile phase A: H₂O+0.1% TFA, mobile phase B: ACN+0.1% TFA. The following gradient of eluent B was used: 27% to 42% over 20 minutes, flow rate 80 mL/minutes, and UV detection at the 214 nm wavelength. Collected fractions were lyophilized to afford Cpd #130.

General procedure L *The peptide was synthesized by standard Solid-phase Peptide Synthesis (SPPS) using Fmoc/t-Bu chemistry. The assembly was performed on a PS Rink-amide MBHA resin (100 umol, 100-200 Mesh; loading 0.42 mmol/g) on the Cem Liberty Blue microwave peptide synthesizer (CEM In.c.). During peptide assembly on solid phase, the side chain of Lys was protected with Dde.

All the amino acids were dissolved at a 0.2 M concentration in DMF. The amino acids were activated with equimolar amounts of a 1 M solution of DIC in DMF and a 1 M solution of Oxyma in DMF. The acylation reactions were performed for 2 minutes at 90° C. under MW irradiation with a 5-fold excess of activated amino acids over the resin free amino groups. The Fmoc deprotection reactions were performed for 3 minutes at 90° C. using a 20% piperidine solution in DMF.

Double acylation reactions were performed for bulky residues (including alpha-methylated, N-methylated or beta-branched amino acids) and following amino acid residue after the bulky one.

N-terminal acetylation was performed for 2 minutes at 65° C. under MW irradiation with a 10% v/v solution of AC₂O in DMF.

Dde protecting group was removed from Lys side chain by treating the resin with a 4% solution of hydrazine monohydrate in DMF (20 mL) over 30 minutes at room temperature.

DOTA was incorporated by manual coupling using an equimolar mixture of DOTA(tBu)₃, DIC and HOBt (2 equiv.) in NMP, overnight at room temperature.

At the end of the assembly the resin was washed with DMF, DCM, Et₂O. The resin bound peptide was cleaved from solid support using 15 mL of TFA solution (v/v: 87.5% TFA, 5% H₂O, 2.5% TIPS, 5% Phenol) for approximately 4 hours, at room temperature. The resin was then filtered and concentrated to about 5 mL and precipitated in cold MTBE (40 mL). After centrifugation, the peptide pellets were washed with fresh cold Et₂O to remove the organic scavengers. The process was repeated twice. Final pellets were dried, re-suspended in H₂O and ACN 1:1 and stirred overnight and then freeze dried to give the crude solid.

The above crude solid was dissolved in a mixture of H2O/ACN (1 mg/mL). TCEP·HCl (3 equiv.) was added, followed by diiodomethane (10 equiv.) and DIPEA (1% v/v). The reaction was stirred at room temperature overnight, then quenched with TFA (5% in water) to adjust pH around 1 and lyophilized. Crude peptide was purified by reverse-phase HPLC using a preparative Waters XBridge C18 column (250×50 mm, 130 Å, 5 μm) with mobile phase A: H₂O+0.1% TFA, mobile phase B: ACN+0.1% TFA. The following gradient of eluent B was used: 27% to 42% over 20 minutes, flow rate 80 mL/minutes, and UV detection at the 214 nm wavelength. Collected fractions were lyophilized to afford the desired product.

### General Procedure M

### Example Cpd #101

Rink Amide MBHA Resin (0.1 mmol, 0.274 g, Sub: 0.365 mmol/g) was swelled in DMF (10 mL)₂ hours, then 20% piperidine in DMF (10 mL) was added into the resin. The mixture was kept at room temperature for 0.5 hours while a stream of nitrogen bubbled through it. The mixture was filtered and the peptidyl resin was washed with DMF (5*10 mL). Fmoc-DLys(Dde)-OH (0.3 mmol, 0.16 g), DMF (1.5 mL), NMM (0.6 mmol, 0.066 mL) and HATU (0.285 mmol, 0.108 g) were added into the resin sequentially. The suspension was kept at room temperature for 1 hour while a stream of nitrogen bubbled through it. The ninhydrine test indicated the coupling completed. The mixture was filtered and the peptidyl resin was washed with DMF (3*10 mL). 20% piperidine in DMF (10 mL) was added into the resin to remove the Fmoc group. The following amino acids were coupled to the resin bound peptide sequentially as follows:

Synthesis Table

| Amino Acid | Reaction Solvent | Reagents | | Reaction Time | Deprotection reagents | |
|---|---|---|---|---|---|---|
| Fmoc-Thr | DMF | HBTU | NMM | 60 | 20% Pip/DMF | 30 |
| (tBu)-OH | (1.5 mL) | 0.285 | 0.6 | minutes | 10 mL | minutes |
| 0.3 mmol | | mmol | mmol | | | |
| Fmoc-Phe-OH | DMF | HBTU | NMM | 60 | 20% Pip/DMF | 30 |
| 0.3 mmol | (1.5 mL) | 0.285 | 0.6 | minutes | 10 mL | minutes |
| | | mmol | mmol | | | |
| Fmoc-Trp | DMF | HBTU | NMM | 60 | 20% Pip/DMF | 30 |
| (Boc)-OH | (1.5 mL) | 0.285 | 0.6 | minutes | 10 mL | minutes |
| 0.3 mmol | | mmol | mmol | | | |
| Fmoc-Cys(Trt)- | DMF | HBTU | NMM | 60 | 20% Pip/DMF | 25 |
| OH 0.3 mmol | (1.5 mL) | 0.285 | 0.6 | minutes | 10 mL | minutes |
| | | mmol | mmol | | | |
| Fmoc- | DMF | HATU | NMM | 60 | 20% Pip/DMF | 30 |
| homoArg(Boc)2- | (1.5 mL) | 0.285 | 0.6 | minutes | 10 mL | minutes |
| OH 0.3 mmol | | mmol | mmol | | | |
| Fmoc-Val-OH | DMF | HBTU | NMM | 60 | 20% Pip/DMF | 30 |
| 0.3 mmol | (1.5 mL) | 0.285 | 0.6 | minutes | 10 mL | minutes |
| | | mmol | mmol | | | |
| Fmoc-Asp(OtBu)- | DMF | HBTU | NMM | 60 | 20% Pip/DMF | 26 |
| OH 0.3 mmol | (1.5 mL) | 0.285 | 0.6 | minutes | 10 mL | minutes |
| | | mmol | mmol | | | |
| Fmoc-Tyr(tBu)- | DMF | HBTU | NMM | 66 | 20% Pip/DMF | 28 |
| OH 0.3 mmol | (1.5 mL) | 0.19 | 0.4 | minutes | 10 mL | minutes |
| | | mmol | mmol | | | |
| Fmoc-Lys(Boc)- | DMF | HBTU | NMM | 85 | 20% Pip/DMF | 30 |
| OH | (1.5 mL) | 0.19 | 0.4 | minutes | 10 mL | minutes |
| 0.3 mmol | | mmol | mmol | | | |
| Fmoc-Gly-OH | DMF | HATU | NMM | 60 | 20% Pip/DMF | 30 |
| 0.6 mmol | (1.5 mL) | 0.57 | 1.2 | minutes | 10 mL | minutes |
| | | mmol | mmol | | | |
| Fmoc-Val-OH | DMF | HATU | NMM | 90 | 20% Pip/DMF | 24 |
| 0.6 mmol | (1.5 mL) | 0.57 | 1.2 | minutes | 10 mL | minutes |
| | | mmol | mmol | | | |
| Fmoc-Cys(Trt)- | DMF | HATU | NMM | 60 | 20% Pip/DMF | 30 |
| OH 0.6 mmol | (1.5 mL) | 0.57 | 1.2 | minutes | 10 mL | minutes |
| | | mmol | mmol | | | |
| Fmoc-Tyr(tBu)- | DMF | HATU | NMM | 60 | 20% Pip/DMF | 30 |
| OH 0.6 mmol | (1.5 mL) | 0.57 | 1.2 | minutes | 10 mL | minutes |
| | | mmol | mmol | | | |
| Fmoc-Nle-OH | DMF | HATU | NMM | 90 | 20% Pip/DMF | 30 |
| 0.6 mmol | (1.5 mL) | 0.57 | 1.2 | minutes | 10 mL | minutes |
| | | mmol | mmol | | | |
| Ac₂O | DMF | N/A | NMM | 30 | 3% hydrazine | 30 |
| 1 mL | (8.4 mL) | | 0.6 | minutes | hydrate/DMF | minutes |
| | | | mL | | 10 mL¹ | |
| DOTA- | DMF | HATU | NMM | 60 | N/A | N/A |
| Tris(tBuester) | (1.5 mL) | 0.285 | 0.6 | minutes | | |
| 0.3 mmol | | mmol | mmol | | | |

After the peptidyl resin was built up, it was washed with DCM (3*10 mL) and MeOH (3*10 mL). The resin was dried under vacuum overnight.

13% hydrazine hydrate in DMF (3 equivalent volume of the resin) was added into the peptidyl resin and the suspension was kept at room temperature while a stream of nitrogen bubbled through it. After 30 minutes, the suspension was filtered and the resin was washed with DMF (3 equivalent volume of the resin) for 5 times.

### Cleavage

Cleavage E solution (TFA:Thioanisole:phenol:EDT:H₂O=87.5:5:2.5:2.5:2.5, 10 mL) was added into peptidyl resin (0.695 g) under the protection of nitrogen. The mixture was shaken for 2.5 hours at room temperature. Cold ether (60 mL) was added into the filtrate, the peptide was precipitated by centrifugation (4000 revolutions/minute). The white precipitation was washed with ether (60 mL) twice. Then the crude was dried under vacuum overnight to give the crude as a white solid (0.26 g, purity: 7%).

Oxidation 0.053 g crude was dissolved by 5 mL ACN and 10 mL H₂O. Then 1 mL of 12 in methanol (10 mg/mL) was added into the mixture and the mixture was stirred for 5 minutes at RT. Ascorbic acid was added into the solution with slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B of 31% to 61% over 60 minutes at a flow rate of 15 mL/minute (solvent A: 4 g/L Ammonium acetate in water; solvent B: 4 g/L Ammonium acetate in 70% ACN/H₂O). The product buffer with purity more than 90% was collected.

### Step 2

The buffer from step 1 with purity more than 90% was purified further by using a Hanbon 300 connected to Phenomenex Luna™ C18 100 Å 10 μm; 21*250 mm HPLC column.

Method: gradient window of solvent B of 34% to 42% over 60 minutes at a flow rate of 15 mL/minute (solvent A: 0.1% TFA in water; solvent B: 0.1% TFA in 80% ACN/H₂O). The left crude was purified by step 1 and step 2, then the solution was combined and lyophilized to give the final peptide (6.7 mg, 96.8%) as a white solid.

### General Procedure M*

### Example Cpd #322

Rink Amide MBHA Resin (0.1 mmol, 0.274 g, Sub: 0.365 mmol/g) was swelled in DMF (10 mL)₂ hours, then 20% piperidine in DMF (10 mL) was added into the resin. The mixture was kept at room temperature for 0.5 hours while a stream of nitrogen bubbled through it. The mixture was filtered and the peptidyl resin was washed with DMF (5*10 mL). Fmoc-hydroxyVal-OH (0.3 mmol, 0.16 g), DMF (1.5 mL), NMM (0.6 mmol, 0.066 mL) and HATU (0.285 mmol, 0.108 g) were added into the resin sequentially. The suspension was kept at room temperature for 1 hour while a stream of nitrogen bubbled through it. The ninhydrine test indicated the coupling completed. The mixture was filtered and the peptidyl resin was washed with DMF (3*10 mL). 20% oiperidine in DMF (10 mL) was added into the resin to remove the Fmoc group. The following amino acids were coupled to the resin bound peptide sequentially as follows:

Synthesis Table

| | Reaction | | | Reaction | | |
|---|---|---|---|---|---|---|
| Amino Acid | Solvent | Reagents | | Time | Deprotection reagents | |
| Fmoc- | DMF | HBTU | NMM | 60 | 20% Pip/DMF | 30 |
| Phe-OH | (1.5 mL) | 0.285 | 0.6 | minutes | 10 mL | minutes |
| 0.3 mmol | | mmol | mmol | | | |
| Fmoc- | DMF | HBTU | NMM | 60 | 20% Pip/DMF | 30 |
| 5FW(Boc)- | (1.5 mL) | 0.285 | 0.6 | minutes | 10 mL | minutes |
| OH | | mmol | mmol | | | |
| 0.3 mmol | | | | | | |
| Fmoc- | DMF | HBTU | NMM | 60 | 20% Pip/DMF | 25 |
| Cys(Trt)- | (1.5 mL) | 0.285 | 0.6 | minutes | 10 mL | minutes |
| OH | | mmol | mmol | | | |
| 0.3 mmol | | | | | | |
| Fmoc- | DMF | HATU | NMM | 60 | 20% Pip/DMF | 30 |
| Tyr(tBu)- | (1.5 mL) | 0.285 | 0.6 | minutes | 10 mL | minutes |
| OH | | mmol | mmol | | | |
| 0.3 mmol | | | | | | |
| Fmoc- | DMF | HBTU | NMM | 60 | 20% Pip/DMF | 30 |
| Val-OH | (1.5 mL) | 0.285 | 0.6 | minutes | 10 mL | minutes |
| 0.3 mmol | | mmol | mmol | | | |
| Fmoc- | DMF | HBTU | NMM | 60 | 20% Pip/DMF | 26 |
| Asp(OtBu)- | (1.5 mL) | 0.285 | 0.6 | minutes | 10 mL | minutes |
| OH | | mmol | mmol | | | |
| 0.3 mmol | | | | | | |
| Fmoc- | DMF | HBTU | NMM | 66 | 20% Pip/DMF | 28 |
| ThpA-OH | (1.5 mL) | 0.19 | 0.4 | minutes | 10 mL | minutes |
| 0.3 mmol | | mmol | mmol | | | |
| Fmoc- | DMF | HBTU | NMM | 85 | 20% Pip/DMF | 30 |
| Lys(Dde)- | (1.5 mL) | 0.19 | 0.4 | minutes | 10 mL | minutes |
| OH | | mmol | mmol | | | |
| 0.3 mmol | | | | | | |
| Fmoc- | DMF | HATU | NMM | 60 | 20% Pip/DMF | 30 |
| Gly-OH | (1.5 mL) | 0.57 | 1.2 | minutes | 10 mL | minutes |
| 0.6 mmol | | mmol | mmol | | | |
| Fmoc- | DMF | HATU | NMM | 90 | 20% Pip/DMF | 24 |
| Tbg-OH | (1.5 mL) | 0.57 | 1.2 | minutes | 10 mL | minutes |
| 0.6 mmol | | mmol | mmol | | | |
| Fmoc- | DMF | HATU | NMM | 60 | 20% Pip/DMF | 30 |
| Cys(Trt)- | (1.5 mL) | 0.57 | 1.2 | minutes | 10 mL | minutes |
| OH 0.6 | | mmol | mmol | | | |
| mmol | | | | | | |
| Fmoc- | DMF | HATU | NMM | 60 | 20% Pip/DMF | 30 |
| Tyr(tBu)- | (1.5 mL) | 0.57 | 1.2 | minutes | 10 mL | minutes |
| OH 0.6 | | mmol | mmol | | | |
| mmol | | | | | | |
| Fmoc- | DMF | HATU | NMM | 90 | 20% Pip/DMF | 30 |
| hLeu-OH | (1.5 mL) | 0.57 | 1.2 | minutes | 10 mL | minutes |
| 0.6 mmol | | mmol | mmol | | | |
| Ac₂O | DMF | N/A | NMM | 30 | 3% hydrazine | 30 |
| 1 mL | (8.4 mL) | | 0.6 | minutes | hydrate/ | minutes |
| | | | mL | | DMF 10 | |
| | | | | | mL | |
| DOTA- | DMF | HATU | NMM | 60 | N/A | N/A |
| Tris(tBuester) | (1.5 mL) | 0.285 | 0.6 | minutes | | |
| 0.3 mmol | | mmol | mmol | | | |

After the peptidyl resin was built up, it was washed with DCM (3*10 mL) and MeOH (3*10 mL). The resin was dried under vacuum overnight.

3% hydrazine hydrate in DMF (3 equivalent volume of the resin) was added into the peptidyl resin and the suspension was kept at room temperature while a stream of nitrogen bubbled through it. After 30 minutes, the suspension was filtered and the resin was washed with DMF (3 equivalent volume of the resin) for 5 times.

### Cleavage

Cleavage E solution (TFA:Thioanisole:phenol:EDT:H₂O=87.5:5:2.5:2.5:2.5, 10 mL) was added into peptidyl resin (0.695 g) under the protection of nitrogen. The mixture was shaken for 2.5 hours at room temperature. Cold ether (60 mL) was added into the filtrate, the peptide was precipitated by centrifugation (4000 revolutions/minute). The white precipitation was washed with ether (60 mL) twice. Then the crude was dried under vacuum overnight to give the crude as a white solid.

### Oxidation

0.053 g crude was dissolved by 5 mL ACN and 10 mL H₂O. Then 1 mL of 12 in methanol (10 mg/mL) was added into the mixture and the mixture was stirred for 5 minutes at RT. Ascorbic acid was added into the solution with slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B of 31% to 61% over 60 minutes at a flow rate of 15 mL/minute (solvent A: 4 g/L Ammonium acetate in water; solvent B: 4 g/L Ammonium acetate in 70% ACN/H₂O). The product buffer with purity more than 90% was collected.

### Step 2

The buffer from step 1 with purity more than 90% was purified further by using a Hanbon 300 connected to Phenomenex Luna™ C18 100 Å 10 μm; 21*250 mm HPLC column. Method: gradient window of solvent B of 34% to 42% over 60 minutes at a flow rate of 15 mL/minute (solvent A: 0.1% TFA in water; solvent B: 0.1% TFA in 80% ACN/H₂O). The left crude was purified by step 1 and step 2, then the solution was combined and lyophilized to give the final peptide as a white solid.

### General Procedure N

### Example Cpd #458

Rink Amide MBHA Resin (0.15 mmol, 0.528 g; Sub: 0.284 mmol/g) was swelled in DMF (10 mL)₂ hours, then 20% piperidine in DMF (10 mL) was added into the resin. The mixture was kept at room temperature for 0.5 hours while a stream of nitrogen bubbled through it. The mixture was filtered and the peptidyl resin was washed with DMF (5*10 mL). Fmoc-DGlu(OtBu)—OH (0.45 mmol, 0.129 g), DMF (1.5 mL), NMM (0.9 mmol, 0.066 mL) and HATU (0.428 mmol, 0.108 g) were added into the resin sequentially. The suspension was kept at room temperature for 1 hour while a stream of nitrogen bubbled through it. The ninhydrine test indicated the coupling completed. The mixture was filtered and the peptidyl resin was washed with DMF (3*10 mL). 20% piperidine in DMF (10 mL) was added into the resin to remove the Fmoc group. The following amino acids were coupled to the resin bound peptide sequentially as follows:

Synthesis Table

| | Reaction | | Reaction | | | |
|---|---|---|---|---|---|---|
| Amino Acid | Solvent | Reagents | Time | Deprotection reagents | | |
| Fmoc-Val(3-OH)-OH | DMF | HATU NMM | 0.6 | 70 | 20% Pip/DMF | 30 |
| 0.3 mmol | (1.5 mL) | 0.285 mmol | mmol | minutes | 10 mL | minutes |
| Fmoc-Phe-OH | DMF | HBTU | NMM | 75 | 20% Pip/DMF | 30 |
| 0.45 mmol | (1.5 mL) | 0.428 | 0.9 | minutes | 10 mL | minutes |
| | | mmol | mmol | | | |
| Fmoc-Trp(5-F)-OH | DMF | HATU | NMM | 60 | 20% Pip/DMF | 30 |
| 0.45 mmol | (1.5 mL) | 0.285 | 0.6 | minutes | 10 mL | minutes |
| | | mmol | mmol | | | |
| Fmoc-Cys(Trt)-OH | DMF | HBTU | NMM | 85 | 20% Pip/DMF | 25 |
| 0.45 mmol | (1.5 mL) | 0.428 | 0.9 | minutes | 10 mL | minutes |
| | | mmol | mmol | | | |
| Fmoc-Tyr(tBu)-OH | DMF | HBTU | NMM | 80 | 20% Pip/DMF | 30 |
| 0.45 mmol | (1.5 mL) | 0.428 | 0.9 | minutes | 10 mL | minutes |
| | | mmol | mmol | | | |
| Fmoc-Val-OH | DMF | HBTU | NMM | 63 | 20% Pip/DMF | 30 |
| 0.45 mmol | (1.5 mL) | 0.428 | 0.9 | minutes | 10 mL | minutes |
| | | mmol | mmol | | | |
| Fmoc-Asp(OtBu)-OH | DMF | HBTU | NMM | 60 | 20% Pip/DMF | 26 |
| 0.45 mmol | (1.5 mL) | 0.428 | 0.9 | minutes | 10 mL | minutes |
| | | mmol | mmol | | | |
| Fmoc-L-2-amino-(1- | DMF | HATU | NMM | 50 | 20% Pip/DMF | 28 |
| Alloc)-4- | (1.5 mL) | 0.285 | 0.6 | minutes | 10 mL | minutes |
| Piperidinepropanoic | | mmol | mmol | | | |
| Acid | | | | | | |
| 0.3 mmol | | | | | | |
| Fmoc-(1-Boc- | DMF | HATU | NMM | 55 | 20% Pip/DMF | 30 |
| piperidin-4-yl)Ala-OH | (1.5 mL) | 0.285 | 0.6 | minutes | 10 mL | minutes |
| 0.3 mmol | | mmol | mmol | | | |
| Fmoc-Gly-OH | DMF | HBTU | NMM | 67 | 20% Pip/DMF | 30 |
| 0.45 mmol | (1.5 mL) | 0.428 | 0.9 | minutes | 10 mL | minutes |
| | | mmol | mmol | | | |
| Fmoc-Tbg-OH | DMF | HATU | NMM | 60 | 20% Pip/DMF | 24 |
| 0.45 mmol | (1.5 mL) | 0.428 | 0.9 | minutes | 10 mL | minutes |
| | | mmol | mmol | | | |
| Fmoc-Cys(Trt)-OH | DMF | HBTU | NMM | 50 | 20% Pip/DMF | 30 |
| 0.45 mmol | (1.5 mL) | 0.428 | 0.9 | minutes | 10 mL | minutes |
| | | mmol | mmol | | | |
| Fmoc-Tyr(tBu)-OH | DMF | HBTU | NMM | 60 | 20% Pip/DMF | 30 |
| 0.45 mmol | (1.5 mL) | 0.428 | 0.9 | minutes | 10 mL | minutes |
| | | mmol | mmol | | | |
| Fmoc-homoLeu-OH | DMF | HATU | NMM | 60 | 20% Pip/DMF | 30 |
| 0.3 mmol | (1.5 mL) | 0.285 | 0.6 | minutes | 10 mL | minutes |
| | | mmol | mmol | | | |
| AC₂O | DMF | N/A | NMM | 30 | 20 eq | 3 hours |
| 1 mL | (8.4 mL) | | 0.6 mL | minutes | phenylsilane | |
| | | | | | and 0.5 equiv. | |
| | | | | | Pd(PPh3)4 to | |
| | | | | | remove Alloc | |
| | | | | | group¹ | |
| Fmoc-AEEP-OH | DMF | HATU | NMM | 85 | N/A | |
| 0.45 mmol | (1.5 mL) | 0.428 | 0.9 | minutes | | |
| | | mmol | mmol | | | |
| Fmoc-AEEP-OH | DMF | HATU | NMM | 19 hours | 20% Pip/DMF | 38 |
| 0.45 mmol | (1.5 mL) | 0.428 | 0.9 | | 10 mL | minutes |
| | | mmol | mmol | | | |
| DOTA-Tris(tBuester) | DMF | HATU | NMM | 60 | N/A | N/A |
| 0.3 mmol | (1.5 mL) | 0.428 | 0.9 | minutes | | |
| | | mmol | mmol | | | |

After the peptidyl resin was built up, it was washed with DCM (3*10 mL) and MeOH (3*10 mL). The resin was dried under vacuum overnight.

The peptidyl reisn was first washed with DCM (3 equivalent volume of the resin) for 3 times and then the suspension was filtered. 0.5 equivalent of Pd(PPh₃)₄, 20 equivalent of phenylsilane and 5 mL DCM were added into the resin sequentially and the reaction was carried out at room temperature for 1.5 hours while a stream of nitrogen bubbled through it, then the suspension was filtered. Usually, the above removal of Alloc procedure was repeated once more to confirm Alloc removed completely. Finally, the resin was washed with 10 mL solution of 0.25% sodium diethyldithiocarbamatre in DMF (1 g sodium diethyldithiocarbamatre dissolved in 400 mL DMF) for 5 minutes and the washing was repeated 6 times until the resin returned to the original color.

### Cleavage

Cleavage E solution (TFA:Thioanisole:phenol:EDT:H₂O=87.5:5:2.5:2.5:2.5, 10 mL) was added into peptidyl resin (1.08 g) under the protection of nitrogen. The mixture was shaken for 2.5 hours at room temperature. Cold ether (60 mL) was added into the filtrate, the peptide was precipitated by centrifugation (4000 revolutions/minute). The white precipitation was washed with ether (60 mL) twice. Then the crude was dried under vacuum overnight to give the crude as a white solid (0.35 g, purity: 27.2%).

### Oxidation

0.133 g crude was dissolved by 10 mL ACN, 15 mL H₂O and 15 mL AcOH. Then 2 mL of 12 in methanol (10 mg/mL) was added into the mixture and the mixture was stirred for 5 minutes at RT. Ascorbic acid was added into the solution with slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B of 38% to 68% over 60 minutes at a flow rate of 15 mL/minute (solvent A: 4 g/L Ammonium acetate in water; solvent B: 4 g/L Ammonium acetate in 70% ACN/H₂O). The product buffer with purity more than 92% was collected.

### Step 2

The buffer from step 1 with purity more than 90% was purified further by using a Hanbon 300 connected to Phenomenex Luna™ C18 100 Å 10 μm; 21*250 mm HPLC column. Method: gradient window of solvent B of 38% to 68% over 60 minutes at a flow rate of 15 mL/minute (solvent A: 0.1% TFA in water; solvent B: 0.1% TFA in 80% ACN/H₂O). The solution was collected and lyophilized to give the final peptide (26.4 mg, 97.3%) as a white solid.

### General Procedure N2

### Example Cpd #496

Rink Amide MBHA Resin (0.1 mmol, 0.353 g, Sub: 0.284 mmol/g) was swelled in DMF (10 mL) for 2 hours, then 20% piperidine in DMF (10 mL) was added into the resin. The mixture was kept at room temperature for 30 minutes while a stream of nitrogen bubbled through it. The mixture was filtered and the peptidyl resin was washed with DMF (5*10 mL). Fmoc-DGlu(OtBu)—OH (0.2 mmol, 0.083 g), DMF (1.5 mL), NMM (0.4 mmol, 0.044 mL) and HATU (0.19 mmol, 0.72 g) were added into the resin sequentially. The suspension was kept at room temperature for 1 hour while a stream of nitrogen bubbled through it. The coupling was monitored by ninhydrine test until its completion. The mixture was filtered and the peptidyl resin was washed with DMF (3*10 mL). 20% piperidine in DMF (10 mL) was added into the resin to remove the Fmoc group. The following amino acids were coupled to the resin bound peptide sequentially as follows:

Synthesis Table

| | Reaction | | | Reaction | Deprotection | |
|---|---|---|---|---|---|---|
| Amino Acid | Solvent | Reagents | | Time | reagents and time | |
| Fmoc-Val(3-OH)-OH | DMF | HATU | NMM | 60 | 20% | 25 |
| 0.2 mmol | (1.5 mL) | 0.19 | 0.4 | minutes | Pip/DMF | minutes |
| | | mmol | mmol | | 10 mL | |
| Fmoc-(2S,3S)-2- | DMF | HATU | NMM | 58 | 20% | 30 |
| amino-3- | (1.5 mL) | 0.19 | 0.4 | minutes | Pip/DMF | minutes |
| phenylbutanoic acid | | mmol | mmol | | 10 mL | |
| 0.2 mmol | | | | | | |
| Fmoc-Trp(5-F)-OH | DMF | HATU | NMM | 55 | 20% | 30 |
| 0.2 mmol | (1.5 mL) | 0.19 | 0.4 | minutes | Pip/DMF | minutes |
| | | mmol | mmol | | 10 mL | |
| Fmoc-Cys(Trt)-OH | DMF | HATU | NMM | 60 | 20% | 25 |
| 0.3 mmol | (1.5 mL) | 0.285 | 0.6 | minutes | Pip/DMF | minutes |
| | | mmol | mmol | | 10 mL | |
| Fmoc-Tyr(tBu)-OH | DMF | HATU | NMM | 59 | 20% | 30 |
| 0.3 mmol | (1.5 mL) | 0.285 | 0.6 | minutes | Pip/DMF | minutes |
| | | mmol | mmol | | 10 mL | |
| Fmoc-Val-OH | DMF | HBTU | NMM | 48 | 20% | 30 |
| 0.3 mmol | (1.5 mL) | 0.285 | 0.6 | minutes | Pip/DMF | minutes |
| | | mmol | mmol | | 10 mL | |
| Fmoc-Asp(OtBu)-OH | DMF | HBTU | NMM | 40 | 20% | 26 |
| 0.3 mmol | (1.5 mL) | 0.285 | 0.6 | minutes | Pip/DMF | minutes |
| | | mmol | mmol | | 10 mL | |
| Fmoc-Ala([DOTA- | DMF | HATU | NMM | 60 | 20% | 28 |
| tris(tBuester)]-PEG4- | (1.5 mL) | 0.142 | 0.3 | minutes | Pip/DMF | minutes |
| piperidin-4-yl)-OH | | mmol | mmol | | 10 mL | |
| 0.15 mmol | | | | | | |
| Fmoc- | DMF | HATU | NMM | 50 | 20% | 30 |
| Pip(CH2COOtBu)Ala- | (1.5 mL) | 0.19 | 0.4 | minutes | Pip/DMF | minutes |
| OH | | mmol | mmol | | 10 mL | |
| 0.2 mmol | | | | | | |
| Fmoc-Gly-OH | DMF | HBTU | NMM | 55 | 20% | 30 |
| 0.3 mmol | (1.5 mL) | 0.285 | 0.6 | minutes | Pip/DMF | minutes |
| | | mmol | mmol | | 10 mL | |
| Fmoc-Tbg-OH | DMF | HATU | NMM | 60 | 20% | 24 |
| 0.2 mmol | (1.5 mL) | 0.19 | 0.4 | minutes | Pip/DMF | minutes |
| | | mmol | mmol | | 10 mL | |
| Fmoc-Cys(Trt)-OH | DMF | HBTU | NMM | 50 | 20% | 30 |
| 0.3 mmol | (1.5 mL) | 0.285 | 0.6 | minutes | Pip/DMF | minutes |
| | | mmol | mmol | | 10 mL | |
| Fmoc-Tyr(tBu)-OH | DMF | HBTU | NMM | 50 | 20% | 30 |
| 0.3 mmol | (1.5 mL) | 0.285 | 0.6 | minutes | Pip/DMF | minutes |
| | | mmol | mmol | | 10 mL | |
| Fmoc-homoLeu-OH | DMF | HATU | NMM | 50 | 20% | 30 |
| 0.3 mmol | (1.5 mL) | 0.285 | 0.6 | minutes | Pip/DMF | minutes |
| | | mmol | mmol | | 10 mL | |
| AC₂O | DMF | N/A | NMM | 30 | N/A | N/A |
| 1 mL | (8.4 mL) | | 0.6 mL | minutes | | |

After the peptidyl resin was assembled, it was washed with DCM (3*10 mL) and MeOH (3*10 mL). The resin was dried under vacuum overnight.

### Cleavage

Cleavage E solution (TFA:Thioanisole:phenol:EDT:H₂O=87.5:5:2.5:2.5:2.5, 10 mL) was added into peptidyl resin (0.782 g) under the protection of nitrogen. The mixture was shaken for 2.5 hours at room temperature. Cold ether (60 mL) was added into the filtrate, the peptide was precipitated by centrifugation (4000 revolutions/minute). The precipitate pellet was washed with ether (60 mL) twice. Then the crude was dried under vacuum overnight to give the crude as a solid. 10

### Oxidation

0.129 g crude was dissolved by 5 mL ACN, 10 mL H₂O and 15 mL AcOH. Then 2 mL of 12 in methanol (10 mg/mL) was added into the mixture and the mixture was stirred for 5 minutes at RT. Ascorbic acid was added into the solution while slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B from 38% to 68% over 60 minutes at a flow rate of 15 mL/minute (solvent A: 4 g/L Ammonium acetate in water; solvent B: 4 g/L Ammonium acetate in 70% ACN/H₂O). The purification fractions with purity more than 91% were collected.

### Step 2

The buffer from step 1 with purity more than 91% was purified further by using a Hanbon 300 connected to Phenomenex Luna™ C18 100 Å 10 μm; 21*250 mm HPLC column. Method: gradient window of solvent B from 38% to 68% over 60 minutes at a flow rate of 15 mL/minute (solvent A: 0.1% TFA in water; solvent B: 0.1% TFA in 80% ACN/H₂O). The solution was collected and lyophilized to give the final peptide as a solid.

### General Procedure O

### Example Cpd #391

Rink Amide MBHA Resin (0.1 mmol, 0.352 g; Sub: 0.284 mmol/g) was swelled in DMF (10 mL)₂ hours, then 20% piperidine in DMF (10 mL) was added into the resin. The mixture was kept at room temperature for 0.5 hours while a stream of nitrogen bubbled through it. The mixture was filtered and the peptidyl resin was washed with DMF (5*10 mL). Fmoc-Val(3-OH)—OH (0.2 mmol, 0.07 g), DMF (1.5 mL), NMM (0.4 mmol, 0.044 mL) and HATU (0.19 mmol, 0.072 g) were added into the resin sequentially. The suspension was kept at room temperature for 1 hour while a stream of nitrogen bubbled through it. The ninhydrine test indicated the coupling completed. The mixture was filtered and the peptidyl resin was washed with DMF (3*10 mL). 20% piperidine in DMF (10 mL) was added into the resin to remove the Fmoc group. The following amino acids were coupled to the resin bound peptide sequentially as follows:

Synthesis Table

| | Re- | | | Re- | | |
|---|---|---|---|---|---|---|
| | action | | | action | Deprotection | |
| Amino Acid | Solvent | Reagents | | Time | reagents | |
| Fmoc-Phe-OH | DMF | HBTU | NMM | 60 min | 20% | 30 |
| 0.3 mmol | (1.5 mL) | 0.285 | 0.6 | | Pip/DMF | min |
| | | mmol | mmol | | 10 mL | |
| Fmoc-Trp(5-F)- | DMF | HATU | NMM | 105 min | 20% | 25 |
| OH 0.2 | (1.5 mL) | 0.19 | 0.4 | | Pip/DMF | min |
| mmol | | mmol | mmol | | 10 mL | |
| Fmoc-Cys(Trt)- | DMF | HBTU | NMM | 60 min | 20% | 28 |
| OH 0.3 | (1.5 mL) | 0.285 | 0.6 | | Pip/DMF | min |
| mmol | | mmol | mmol | | 10 mL | |
| Fmoc-Tyr(tBu)- | DMF | HBTU | NMM | 60 min | 20% | 30 |
| OH 0.3 | (1.5 mL) | 0.285 | 0.6 | | Pip/DMF | min |
| mmol | | mmol | mmol | | 10 mL | |
| Fmoc-Val-OH | DMF | HBTU | NMM | 60 min | 20% | 25 |
| 0.3 mmol | (1.5 mL) | 0.285 | 0.6 | | Pip/DMF | min |
| | | mmol | mmol | | 10 mL | |
| Fmoc-Asp(OtBu)- | DMF | HBTU | NMM | 80 min | 20% | 25 |
| OH 0.3 mmol | (1.5 mL) | 0.285 | 0.6 | | Pip/DMF | min |
| | | mmol | mmol | | 10 mL | |
| Fmoc-L- | DMF | HATU | NMM | 57 min | 20% | 30 |
| Ala(tetrahydropyran- | (1.5 mL) | 0.19 | 0.4 | | Pip/DMF | min |
| 4-yl)-OH | | mmol | mmol | | 10 mL | |
| 0.2 mmol | | | | | | |
| Fmoc-Lys(Boc)- | DMF | HBTU | NMM | 60 min | | 30 |
| OH 0.3 | (1.5 mL) | 0.285 | 0.6 | | | min |
| mmol | | mmol | mmol | | | |
| Fmoc-Gly-OH | DMF | HBTU | NMM | 50 min | 20% | 27 |
| 0.3 mmol | (1.5 mL) | 0.285 | 0.6 | | Pip/DMF | min |
| | | mmol | mmol | | 10 mL | |
| Fmoc-Tbg-OH | DMF | HATU | NMM | 61 min | 20% | 30 |
| 0.3 mmol | (1.5 mL) | 0.285 | 0.6 | | Pip/DMF | min |
| | | mmol | mmol | | 10 mL | |
| Fmoc-Cys(Trt)- | DMF | HBTU | NMM | 60 min | 20% | 30 |
| OH 0.3 mmol | (1.5 mL) | 0.285 | 0.6 | | Pip/DMF | min |
| | | mmol | mmol | | 10 mL | |
| Fmoc-Tyr(tBu)- | DMF | HBTU | NMM | 65 min | 20% | 30 |
| OH 0.3 mmol | (1.5 mL) | 0.285 | 0.6 | | Pip/DMF | min |
| | | mmol | mmol | | 10 mL | |
| Fmoc-homoLeu-OH | DMF | HATU | NMM | 60 min | 20% | 30 |
| 0.2 mmol | (1.5 mL) | 0.19 | 0.4 | | Pip/DMF | min |
| | | mmol | mmol | | 10 mL | |
| Fmoc-PEG8-OH | DMF | HATU | NMM | 97 min | 20% | 20 |
| 0.2 mmol | (1.5 mL) | 0.19 | 0.4 | | Pip/DMF | min |
| | | mmol | mmol | | 10 mL | |
| DOTA-Tris(tBuester) | DMF | HATU | NMM | 50 min | N/A | N/A |
| 0.3 mmol | (1.5 mL) | 0.285 | 0.6 | | | |
| | | mmol | mmol | | | |

After the peptidyl resin was built up, it was washed with DCM (3*10 mL) and MeOH (3*10 mL). The resin was dried under vacuum overnight.

### Cleavage

Cleavage E solution (TFA:Thioanisole:phenol:EDT:H₂O=87.5:5:2.5:2.5:2.5, 10 mL) was added into peptidyl resin (0.817 g) under the protection of nitrogen. The mixture was shaken for 2.5 hours at room temperature. Cold ether (60 mL) was added into the filtrate, the peptide was precipitated by centrifugation (4000 revolutions/minute). The white precipitation was washed with ether (60 mL) twice. Then the crude was dried under vacuum overnight to give the crude as a white solid (0.263 g, purity: 32%).

### Oxidation

0.109 g crude was dissolved by 10 mL ACN, 10 mL H₂O and 15 mL AcOH. Then 2 mL of 12 in methanol (10 mg/mL) was added into the mixture and the mixture was stirred for 5 minutes at RT. Ascorbic acid was added into the solution with slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B of 47% to 77% over 60 minutes at a flow rate of 15 mL/minute (solvent A: 4 g/L Ammonium acetate in water; solvent B: 4 g/L Ammonium acetate in 70% ACN/H₂O). The product buffer with purity more than 92% was collected.

### Step 2

The buffer from step 1 with purity more than 90% was purified further by using a Hanbon 300 connected to Phenomenex Luna™ C18 100 Å 10 μm; 21*250 mm HPLC column. Method: gradient window of solvent B of 38% to 68% over 60 minutes at a flow rate of 15 mL/minute (solvent A: 0.1% TFA in water; solvent B: 0.1% TFA in 80% ACN/H₂O). Then the solution was combined and lyophilized to give the final peptide (18.5 mg, 97.9%) as a white solid.

### General Procedure P

### Example Cpd #389

CTC Resin (0.5 g, Sub: 0.97 mmol/g) was swelled in DMF (10 mL)₂ hours, Fmoc-DLys(Dde)-OH (0.106 g, 0.2 mmol) and DIPEA (0.42 mL, 2.5 mmol) were added into the suspension. The mixture was kept at room temperature for 3 hours while a stream of nitrogen bubbled through it. Then 1 mL MeOH was added into the mixture and resulting mixture was kept at room temperature for 0.5 hours. Reaction mixture was filtered. The resin was washed with DMF (3*10 mL) and then MeOH (3*10 mL).

The resin was swelled in DMF (10 mL) for 2 hours, then 20% piperidine in DMF (10 mL) was added into the resin. The mixture was kept at room temperature for 0.5 hours while a stream of nitrogen bubbled through it. The mixture was filtered and the peptidyl resin was washed with DMF (5*10 mL). Fmoc-Val(3-OH)—OH (0.2 mmol, 0.07 g), DMF (1.5 mL), NMM (0.4 mmol, 0.044 mL) and HATU (0.19 mmol, 0.072 g) were added into the resin sequentially. The suspension was kept at room temperature for 1 hour while a stream of nitrogen bubbled through it. The ninhydrine test indicated the coupling completed. The mixture was filtered and the peptidyl resin was washed with DMF (3*10 mL). 20% piperidine in DMF (10 mL) was added into the resin to remove the Fmoc group. The following amino acids were coupled to the resin bound peptide sequentially as follows:

Synthesis Table

| | Reaction | | | Reaction | | |
|---|---|---|---|---|---|---|
| Amino Acid | Solvent | Reagents | | Time | Deprotection reagents | |
| Fmoc-Phe-OH | DMF | HBTU | NMM | 70 min | 20% Pip/DMF 10 mL | 20 min |
| 0.3 mmol | (1.5 mL) | 0.285 | 0.6 | | | |
| | | mmol | mmol | | | |
| Fmoc-Trp(5-F)-OH | DMF | HATU | NMM | 60 min | 20% Pip/DMF 10 mL | 30 min |
| 0.2 mmol | (1.5 mL) | 0.19 | 0.4 | | | |
| | | mmol | mmol | | | |
| Fmoc-Cys(Trt)-OH | DMF | HBTU | NMM | 60 min | 20% Pip/DMF 10 mL | 30 min |
| 0.3 mmol | (1.5 mL) | 0.285 | 0.6 | | | |
| | | mmol | mmol | | | |
| Fmoc-Tyr(OSO2F)- | DMF | HATU | NMM | 63 min | 20% 2- | 30 min |
| OH 0.2 mmol | (1.5 mL) | 0.19 | 0.4 | | Methylpiperidine/DMF | |
| | | mmol | mmol | | 10 mL | |
| Fmoc-Val-OH | DMF | HBTU | NMM | 59 min | 20% 2- | 24 min |
| 0.6 mmol | (1.5 mL) | 0.57 | 1.2 | | Methylpiperidine/DMF | |
| | | mmol | mmol | | 10 mL | |
| Fmoc-Asp(OtBu)- | DMF | HBTU | NMM | 61 min | 20% 2- | 25 min |
| OH 0.3 mmol | (1.5 mL) | 0.285 | 0.6 | | Methylpiperidine/DMF | |
| | | mmol | mmol | | 10 mL | |
| Fmoc-L- | DMF | HATU | NMM | 60 min | 20% 2- | 24 min |
| Ala(tetrahydropyran- | (1.5 mL) | 0.19 | 0.4 | | Methylpiperidine/DMF | |
| 4-yl)-OH | | mmol | mmol | | 10 mL | |
| 0.2 mmol | | | | | | |
| Fmoc-Lys(Boc)-OH | DMF | HBTU | NMM | 75 min | 20% 2- | 20 min |
| 0.3 mmol | (1.5 mL) | 0.285 | 0.6 | | Methylpiperidine/DMF | |
| | | mmol | mmol | | 10 mL | |
| Fmoc-Gly-OH | DMF | HBTU | NMM | 50 min | 20% 2- | 23 min |
| 0.3 mmol | (1.5 mL) | 0.285 | 0.6 | | Methylpiperidine/DMF | |
| | | mmol | mmol | | 10 mL | |
| Fmoc-Tbg-OH | DMF | HATU | NMM | 61 min | 20% 2- | 30 min |
| 0.3 mmol | (1.5 mL) | 0.285 | 0.6 | | Methylpiperidine/DMF | |
| | | mmol | mmol | | 10 mL | |
| Fmoc-Cys(Trt)-OH | DMF | HBTU | NMM | 50 min | 20% 2- | 20 min |
| 0.3 mmol | (1.5 mL) | 0.285 | 0.6 | | Methylpiperidine/DMF | |
| | | mmol | mmol | | 10 mL | |
| Fmoc-Tyr(tBu)-OH | DMF | HBTU | NMM | 61 min | 20% 2- | 28 min |
| 0.3 mmol | (1.5 mL) | 0.285 | 0.6 | | Methylpiperidine/DMF | |
| | | mmol | mmol | | 10 mL | |
| Fmoc-homoLeu-OH | DMF | HATU | NMM | 55 min | 20% 2- | 25 min |
| 0.3 mmol | (1.5 mL) | 0.285 | 0.6 | | methylpiperidine/DMF | |
| | | mmol | mmol | | 10 mL | |
| AC₂O 1 mL | DMF | N/A | NMM | 33 min | 3% hydrazine hydrate/DMF | 27 min |
| | (8.4 mL) | | 0.6 | | to remove Dde group¹ | |
| | | | mL | | | |
| DOTA- | DMF | HATU | NMM | 60 min | N/A | N/A |
| Tris(tBuester) | (1.5 mL) | 0.285 | 0.6 | | | |
| 0.3 mmol | | mmol | mmol | | | |

After the peptidyl resin was built up, it was washed with DCM (3×10 mL) and MeOH (3*10 mL). The resin was dried under vacuum overnight.

3% hydrazine hydrate in DMF (3 equivalent volume of the resin) was added into the peptidyl resin and the suspension was kept at room temperature while a stream of nitrogen bubbled through it. After 30 minutes, the suspension was filtered and the resin was washed with DMF (3 equivalent volume of the resin) for 5 times.

### Cleavage

Cleavage E solution (TFA:Thioanisole:phenol:EDT:H₂O=87.5:5:2.5:2.5:2.5, 10 mL) was added into peptidyl resin (1.1 g) under the protection of nitrogen. The mixture was shaken for 2.5 hours at room temperature. Cold ether (60 mL) was added into the filtrate, the peptide was precipitated by centrifugation (4000 revolutions/minute). The white precipitation was washed with ether (60 mL) twice. Then the crude was dried under vacuum overnight to give the crude as a white solid (0.37 g, purity: 13%).

### Oxidation

0.37 g crude was dissolved by 20 mL ACN, 20 mL H₂O and 20 mL AcOH. Then 5 mL of 12 in methanol (10 mg/mL) was added into the mixture and the mixture was stirred for 5 minutes at RT. Ascorbic acid was added into the solution with slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B of 40% to 70% over 60 minutes at a flow rate of 15 mL/minute (solvent A: 4 g/L Ammonium acetate in water; solvent B: 4 g/L Ammonium acetate in 70% ACN/H₂O). The product buffer with purity more than 92% was collected.

### Step 2

The buffer from step 1 with purity more than 90% was purified further by using a Hanbon 300 connected to Phenomenex Luna™ C18 100 Å 10 μm; 21*250 mm HPLC column. Method: gradient window of solvent B of 40% to 70% over 60 minutes at a flow rate of 15 mL/minute (solvent A: 0.1% TFA in water; solvent B: 0.1% TFA in 80% ACN/H₂O). The solution was collected and lyophilized to give the final peptide (6.5 mg, 93%) as a white solid.

### General Procedure S

### Example Cpd #517

CTC Resin (0.5 g, Sub: 0.69 mmol/g) was swelled in DMF (10 mL)₂ hours, Dde-Lys(Fmoc)-OH (0.106 g, 0.2 mmol) and DIPEA (0.42 mL, 2.5 mmol) were added into the suspension. The mixture was kept at room temperature for 3 hours while a stream of nitrogen bubbled through it. Then 1 mL MeOH was added into the mixture and resulting mixture was kept at room temperature for 0.5 hours. After the reaction mixture was filtered, the resin was washed with DMF (3*10 mL) and then MeOH (3*10 mL).

The resin was swelled in DMF (10 mL) for 2 hours, then 20% piperidine in DMF (10 mL) was added into the resin. The mixture was kept at room temperature for 0.5 hours while a stream of nitrogen bubbled through it. The mixture was filtered and the peptidyl resin was washed with DMF (5*10 mL). Fmoc-Val(3-OH)—OH (0.2 mmol, 0.07 g), DMF (1.5 mL), NMM (0.4 mmol, 0.044 mL) and HATU (0.19 mmol, 0.072 g) were added into the resin sequentially. The suspension was kept at room temperature for 1 hour while a stream of nitrogen bubbled through it. The ninhydrine test indicated the coupling completed. The mixture was filtered and the peptidyl resin was washed with DMF (3*10 mL). 20% piperidine in DMF (10 mL) was added into the resin to remove the Fmoc group. The following amino acids were coupled to the resin bound peptide sequentially as follows:

Synthesis Table

| | Reaction | | | Reaction | | |
|---|---|---|---|---|---|---|
| Amino Acid | Solvent | Reagents | | Time | Deprotection reagents | |
| Fmoc-(2S,3S)-2- | DMF | HATU | NMM | 60 min | 20% Pip/DMF 10 | 30 min |
| amino-3- | (1.5 mL) | 0.19 | 0.4 | | mL | |
| phenylbutanoic acid | | mmol | mmol | | | |
| 0.2 mmol | | | | | | |
| Fmoc-Trp(5-F)-OH | DMF | HATU | NMM | 60 min | 20% Pip/DMF 10 | 30 min |
| 0.2 mmol | (1.5 mL) | 0.19 | 0.4 | | mL | |
| | | mmol | mmol | | | |
| Fmoc-Cys(Trt)-OH | DMF | HBTU | NMM | 60 min | 20% Pip/DMF 10 | 30 min |
| 0.3 mmol | (1.5 mL) | 0.285 | 0.6 | | mL | |
| | | mmol | mmol | | | |
| Fmoc-Tyr(tBu)-OH | DMF | HBTU | NMM | 54 min | 20% Pip/DMF 10 | 30 min |
| 0.3 mmol | (1.5 mL) | 0.285 | 0.6 | | mL | |
| | | mmol | mmol | | | |
| Fmoc-Val-OH | DMF | HBTU | NMM | 50 min | 20% Pip/DMF 10 | 30 min |
| 0.3 mmol | (1.5 mL) | 0.285 | 0.6 | | mL | |
| | | mmol | mmol | | | |
| Fmoc-Asp(OtBu)- | DMF | HBTU | NMM | 60 min | 20% Pip/DMF 10 | 30 min |
| OH | (1.5 mL) | 0.285 | 0.6 | | mL | |
| 0.3 mmol | | mmol | mmol | | | |
| Fmoc-L- | DMF | HATU | NMM | 61 min | 20% Pip/DMF 10 | 30 min |
| Ala(tetrahydropyran- | (1.5 mL) | 0.19 | 0.4 | | mL | |
| 4-yl)-OH | | mmol | mmol | | | |
| 0.2 mmol | | | | | | |
| Fmoc-(1-Boc- | DMF | HATU | NMM | 58 min | 20% Pip/DMF 10 | 30 min |
| piperidin-4-yl)Ala- | (1.5 mL) | 0.19 | 0.4 | | mL | |
| OH | | mmol | mmol | | | |
| 0.2 mmol | | | | | | |
| Fmoc-Gly-OH | DMF | HBTU | NMM | 50 min | 20% Pip/DMF 10 | 30 min |
| 0.3 mmol | (1.5 mL) | 0.285 | 0.6 | | mL | |
| | | mmol | mmol | | | |
| Fmoc-Tbg-OH | DMF | HATU | NMM | 50 min | 20% Pip/DMF 10 | 30 min |
| 0.3 mmol | (1.5 mL) | 0.285 | 0.6 | | mL | |
| | | mmol | mmol | | | |
| Fmoc-Cys(Trt)-OH | DMF | HBTU | NMM | 60 min | 20% Pip/DMF 10 | 30 min |
| 0.3 mmol | (1.5 mL) | 0.285 | 0.6 | | mL | |
| | | mmol | mmol | | | |
| Fmoc-Tyr(tBu)-OH | DMF | HATU | NMM | 60 min | 20% Pip/DMF 10 | 30 min |
| 0.6 mmol | (1.5 mL) | 0.57 | 1.2 | | mL | |
| | | mmol | mmol | | | |
| Fmoc-homoLeu- OH | DMF | HATU | NMM | 80 min | 20% Pip/DMF 10 | 30 min |
| 0.3 mmol | (1.5 mL) | 0.285 | 0.6 | | mL | |
| | | mmol | mmol | | | |
| AC₂O | DMF | N/A | NMM | 30 min | 3% hydrazine | 30 min |
| 1 mL | (8.4 mL) | | 0.6 mL | | hydrate/DMF 10 | |
| | | | | | mL¹ | |
| Fmoc-Val-OH | DMF | HBTU | NMM | 50 min | 20% Pip/DMF 10 | 30 min |
| 0.6 mmol | (1.5 mL) | 0.57 | 1.2 | | mL | |
| | | mmol | mmol | | | |
| Fmoc-Met-OH | DMF | HBTU | NMM | 60 min | 20% Pip/DMF 10 | 30 min |
| 0.6 mmol | (1.5 mL) | 0.57 | 1.2 | | mL | |
| | | mmol | mmol | | | |
| Fmoc-(4- | DMF | HATU | NMM | 60 min | 20% Pip/DMF 10 | 30 min |
| aminomethyl) | (1.5 mL) | 0.285 | 0.6 | | mL | |
| benzoic acid | | mmol | mmol | | | |
| 0.3 mmol | | | | | | |
| DOTA- | DMF | HATU | NMM | 59 min | N/A | N/A |
| Tris(tBuester) | (1.5 mL) | 0.285 | 0.6 | | | |
| 0.3 mmol | | mmol | mmol | | | |

After the peptidyl resin was built up, it was washed with DCM (3×10 mL) and MeOH (3*10 mL). The resin was dried under vacuum overnight.

3% hydrazine hydrate in DMF (3 equivalent volume of the resin) was added into the peptidyl resin and the suspension was kept at room temperature while a stream of nitrogen bubbled through it. After 30 minutes, the suspension was filtered and the resin was washed with DMF (3 equivalent volume of the resin) for 5 times.

### Cleavage

Cleavage E solution (TFA:Thioanisole:phenol:EDT:H₂O=87.5:5:2.5:2.5:2.5, 10 mL) was added into peptidyl resin (0.873 g) under the protection of nitrogen. The mixture was shaken for 2.5 hours at room temperature. Cold ether (60 mL) was added into the filtrate, the peptide was precipitated by centrifugation (4000 revolutions/minute). The white precipitation was washed with ether (60 mL) twice. Then the crude was dried under vacuum overnight to give the crude as a white solid (0.275 g, purity: 44.8%).

### Oxidation

0.275 g crude was dissolved by 10 mL ACN, 10 mL H₂O and 15 mL AcOH. Then 5 mL of 12 in methanol (10 mg/mL) was added into the mixture and the mixture was stirred for 5 minutes at RT. Ascorbic acid was added into the solution with slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B from 42% to 72% over 60 minutes at a flow rate of 15 mL/minute (solvent A: 4 g/L Ammonium acetate in water; solvent B: 4 g/L Ammonium acetate in 70% ACN/H₂O). The product buffer with purity more than 93% was collected.

### Step 2

The buffer from step 1 with purity more than 93% was purified further by using a Hanbon 300 connected to Phenomenex Luna™ C18 100 Å 10 μm; 21*250 mm HPLC column. Method: gradient window of solvent B from 40% to 70% over 60 minutes at a flow rate of 15 mL/minute (solvent A: 0.1% TFA in water; solvent B: 0.1% TFA in 80% ACN/H₂O). The solution was collected and lyophilized to give the final peptide (28.7 mg, 95.4%) as a white solid.

### General Procedure S*

### Example Cpd #518

The Fmoc-DSer(tBu)-CTC resin (Sub: 0.258 mmol/g, 0.1 mmol) was swelled in DMF (10 mL) for 2 hours, then 20% piperidine in DMF (10 mL) was added into the resin. The mixture was kept at room temperature for 0.5 hours while a stream of nitrogen bubbled through it. The mixture was filtered and the peptidyl resin was washed with DMF (5*10 mL). Fmoc-Val(3-OH)—OH (0.2 mmol, 0.07 g), DMF (1.5 mL), NMM (0.4 mmol, 0.044 mL) and HATU (0.19 mmol, 0.072 g) were added into the resin sequentially. The suspension was kept at room temperature for 1 hour while a stream of nitrogen bubbled through it. The ninhydrine test indicated the coupling completed. The mixture was filtered and the peptidyl resin was washed with DMF (3*10 mL). 20% piperidine in DMF (10 mL) was added into the resin to remove the Fmoc group. The following amino acids were coupled to the resin bound peptide sequentially as follows:

Synthesis Table

| | Reaction | | | Reaction | | |
|---|---|---|---|---|---|---|
| Amino Acid | Solvent | Reagents | | Time | Deprotection reagents | |
| Fmoc-(2S,3S)-2- | DMF | HATU | NMM | 50 min | 20% Pip/DMF 10 | 30 min |
| amino-3- | (1.5 mL) | 0.19 mmol | 0.4 | | mL | |
| phenylbutanoic | | | mmol | | | |
| acid | | | | | | |
| 0.2 mmol | | | | | | |
| Fmoc-Trp(5-F)- | DMF | HATU | NMM | 60 min | 20% Pip/DMF 10 | 30 min |
| OH | (1.5 mL) | 0.19 mmol | 0.4 | | mL | |
| 0.2 mmol | | | mmol | | | |
| Fmoc-Cys(Trt)- | DMF | HBTU | NMM | 60 min | 20% Pip/DMF 10 | 30 min |
| OH | (1.5 mL) | 0.285 | 0.6 | | mL | |
| 0.3 mmol | | mmol | mmol | | | |
| Fmoc-Tyr(tBu)- | DMF | HBTU | NMM | 58 min | 20% Pip/DMF 10 | 30 min |
| OH | (1.5 mL) | 0.285 | 0.6 | | mL | |
| 0.3 mmol | | mmol | mmol | | | |
| Fmoc-Val-OH | DMF | HBTU | NMM | 50 min | 20% Pip/DMF 10 | 30 min |
| 0.3 mmol | (1.5 mL) | 0.285 | 0.6 | | mL | |
| | | mmol | mmol | | | |
| Fmoc- | DMF | HBTU | NMM | 60 min | 20% Pip/DMF 10 | 30 min |
| Asp(OtBu)-OH | (1.5 mL) | 0.285 | 0.6 | | mL | |
| 0.3 mmol | | mmol | mmol | | | |
| Fmoc-Glu(OAll)- | DMF | HATU | NMM | 61 min | 20% Pip/DMF 10 | 30 min |
| OH | (1.5 mL) | 0.19 mmol | 0.4 | | mL | |
| 0.2 mmol | | | mmol | | | |
| Fmoc-(1-Boc- | DMF | HATU | NMM | 58 min | 20% Pip/DMF 10 | 30 min |
| piperidin-4- | (1.5 mL) | 0.19 mmol | 0.4 | | mL | |
| yl)Ala-OH | | | mmol | | | |
| 0.2 mmol | | | | | | |
| Fmoc-Gly-OH | DMF | HBTU | NMM | 50 min | 20% Pip/DMF 10 | 30 min |
| 0.3 mmol | (1.5 mL) | 0.285 | 0.6 | | mL | |
| | | mmol | mmol | | | |
| Fmoc-Tbg-OH | DMF | HATU | NMM | 50 min | 20% Pip/DMF 10 | 30 min |
| 0.3 mmol | (1.5 mL) | 0.285 | 0.6 | | mL | |
| | | mmol | mmol | | | |
| Fmoc-Cys(Trt)- | DMF | HBTU | NMM | 60 min | 20% Pip/DMF 10 | 30 min |
| OH | (1.5 mL) | 0.285 | 0.6 | | mL | |
| 0.3 mmol | | mmol | mmol | | | |
| Fmoc-Tyr(tBu)- | DMF | HATU | NMM | 65 min | 20% Pip/DMF 10 | 30 min |
| OH | (1.5 mL) | 0.57 mmol | 1.2 | | mL | |
| 0.6 mmol | | | mmol | | | |
| Fmoc-homoLeu- | DMF | HATU | NMM | 80 min | 20% Pip/DMF 10 | 30 min |
| OH | (1.5 mL) | 0.285 | 0.6 | | mL | |
| 0.3 mmol | | mmol | mmol | | | |
| AC₂O | DMF | N/A | NMM | 30 min | N/A | N/A |
| 1 mL | (8.4 mL) | | 0.6 mL | | | |
| ¹De-OAll | DCM | Pd(PPh3)4 | NMM | 3 h | N/A | N/A |
| | 5 mL | 0.3 mmol | 0.1 mL | | | |
| Fmoc-Lys- | DMF | HATU | NMM | 80 min | 20% Pip/DMF 10 | 30 min |
| OtBu.HCl | (1.5 mL) | 0.285 | 0.6 | | mL | |
| 0.3 mmol | | mmol | mmol | | | |
| Fmoc-Val-OH | DMF | HATU | NMM | 60 min | 20% Pip/DMF 10 | 30 min |
| 0.3 mmol | (1.5 mL) | 0.285 | 0.6 | | mL | |
| | | mmol | mmol | | | |
| Fmoc-Met-OH | DMF | HATU | NMM | 60 min | 20% Pip/DMF 10 | 30 min |
| 0.3 mmol | (1.5 mL) | 0.285 | 0.6 | | mL | |
| | | mmol | mmol | | | |
| Fmoc-(4- | DMF | HATU | NMM | 60 min | 20% Pip/DMF 10 | 30 min |
| aminomethyl) | (1.5 mL) | 0.285 | 0.6 | | mL | |
| benzoic acid | | mmol | mmol | | | |
| 0.2 mmol | | | | | | |
| DOTA- | DMF | HATU | NMM | 45 min | N/A | N/A |
| Tris(tBuester) | (1.5 mL) | 0.19 mmol | 0.6 | | | |
| 0.2 mmol | | | mmol | | | |

After the peptidyl resin was built up, it was washed with DCM (3×10 mL) and MeOH (3*10 mL). The resin was dried under vacuum overnight.

OAll deprotection: The peptidyl reisn was first washed with DCM (3 equivalent volume of the resin) for 3 times and then the suspension was filtered. 3 equivalent of Pd(PPh₃)₄ and 5 mL DCM were added into the resin sequentially and the reaction was carried out at room temperature for 1.5 hours while a stream of nitrogen bubbled through it, then the suspension was filtered. The above removal of OAll procedure was repeated once more to confirm OAll removed completely.

Finally, the resin was washed with 10 mL solution of 0.25% sodium diethyldithiocarbamatre in DMF (1 g sodium diethyldithiocarbamatre dissolved in 400 mL DMF) for 5 min and the washing was repeated 6 times until the resin returned to the original color.

### Cleavage

Cleavage E solution (TFA:Thioanisole:phenol:EDT:H₂O=87.5:5:2.5:2.5:2.5, 10 mL) was added into peptidyl resin (0.577 g) under the protection of nitrogen. The mixture was shaken for 2.5 hours at room temperature. Cold ether (60 mL) was added into the filtrate, the peptide was precipitated by centrifugation (4000 revolutions/minute). The white precipitation was washed with ether (60 mL) twice. Then the crude was dried under vacuum overnight to give the crude as a white solid (0.167 g, purity: 26.1%).

### Oxidation

0.167 g crude was dissolved by 10 mL ACN, 15 mL H₂O and 15 mL AcOH. Then 2.5 mL of 12 in methanol (10 mg/mL) was added into the mixture and the mixture was stirred for 5 minutes at RT. Ascorbic acid was added into the solution with slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B from 42% to 72% over 60 minutes at a flow rate of 15 mL/minute (solvent A: 4 g/L Ammonium acetate in water; solvent B: 4 g/L Ammonium acetate in 70% ACN/H₂O). The product buffer with purity more than 92% was collected.

### Step 2

The buffer from step 1 with purity more than 92% was purified further by using a Hanbon 300 connected to Phenomenex Luna™ C18 100 Å 10 μm; 21*250 mm HPLC column. Method: gradient window of solvent B from 38% to 68% over 60 minutes at a flow rate of 15 mL/minute (solvent A: 0.1% TFA in water; solvent B: 0.1% TFA in 80% ACN/H₂O). The solution was collected and lyophilized to give the final peptide (21.2 mg, 95.0%) as a white solid.

### General Procedure T

### Example Cpd #468

The Fmoc-DLys(Dde)-CTC Resin (Sub: 0.194 mmol/g, 0.1 mmol, 0.516 g) was swelled in DMF (10 mL) for 2 hours, then 20% piperidine in DMF (10 mL) was added into the resin. The mixture was kept at room temperature for 0.5 hours while a stream of nitrogen bubbled through it. The mixture was filtered and the peptidyl resin was washed with DMF (5*10 mL). Fmoc-Val(3-OH)—OH (0.2 mmol, 0.071 g), DMF (1.5 mL), NMM (0.4 mmol, 0.044 mL) and HATU (0.19 mmol, 0.072 g) were added into the resin sequentially. The suspension was kept at room temperature for 1 hour while a stream of nitrogen bubbled through it. The ninhydrine test indicated the coupling completed. The mixture was filtered and the peptidyl resin was washed with DMF (3*10 mL). 20% piperidine in DMF (10 mL) was added into the resin to remove the Fmoc group. The following amino acids were coupled to the resin bound peptide sequentially as follows:

Synthesis Table

| | Reaction | | | Reaction | | |
|---|---|---|---|---|---|---|
| Amino Acid | Solvent | Reagents | | Time | Deprotection reagents | |
| Fmoc-Phe-OH | DMF | HBTU | NMM | 50 min | 20% Pip/DMF 10 | 30 |
| 0.3 mmol | (1.5 mL) | 0.285 | 0.6 | | mL | min |
| | | mmol | mmol | | | |
| Fmoc-Trp(5-F)-OH | DMF | HATU | NMM | 50 min | 20% Pip/DMF 10 | 30 |
| 0.2 mmol | (1.5 mL) | 0.19 | 0.4 | | mL | min |
| | | mmol | mmol | | | |
| Fmoc-Cys(Trt)-OH | DMF | HBTU | NMM | 50 min | 20% Pip/DMF 10 | 30 |
| 0.3 mmol | (1.5 mL) | 0.285 | 0.6 | | mL | min |
| | | mmol | mmol | | | |
| Fmoc-Tyr(tBu)-OH | DMF | HBTU | NMM | 60 min | 20% Pip/DMF 10 | 30 |
| 0.3 mmol | (1.5 mL) | 0.285 | 0.6 | | mL | min |
| | | mmol | mmol | | | |
| Fmoc-Val-OH | DMF | HBTU | NMM | 50 min | 20% Pip/DMF 10 | 30 |
| 0.3 mmol | (1.5 mL) | 0.285 | 0.6 | | mL | min |
| | | mmol | mmol | | | |
| Fmoc-Asp(OtBu)- | DMF | HBTU | NMM | 45 min | 20% Pip/DMF 10 | 22 |
| OH | (1.5 mL) | 0.285 | 0.6 | | mL | min |
| 0.3 mmol | | mmol | mmol | | | |
| Fmoc-L- | DMF | HATU | NMM | 50 min | 20% Pip/DMF 10 | 30 |
| Ala(tetrahydropyran- | (1.5 mL) | 0.285 | 0.6 | | ml | min |
| 4-yl)-OH | | mmol | mmol | | | |
| 0.3 mmol | | | | | | |
| Fmoc-(1-Boc- | DMF | HATU | NMM | 60 min | 20% Pip/DMF 10 | 30 |
| piperidin-4-yl)Ala- | (1.5 mL) | 0.19 | 0.4 | | mL | min |
| OH | | mmol | mmol | | | |
| 0.2 mmol | | | | | | |
| Fmoc-Gly-OH | DMF | HBTU | NMM | 60 min | 20% Pip/DMF 10 | 30 |
| 0.3 mmol | (1.5 mL) | 0.285 | 0.6 | | mL | min |
| | | mmol | mmol | | | |
| Fmoc-Tbg-OH | DMF | HATU | NMM | 60 min | 20% Pip/DMF 10 | 24 |
| 0.3 mmol | (1.5 mL) | 0.285 | 0.6 | | mL | min |
| | | mmol | mmol | | | |
| Fmoc-Cys(Trt)-OH | DMF | HBTU | NMM | 60 min | 20% Pip/DMF 10 | 20 |
| 0.3 mmol | (1.5 mL) | 0.285 | 0.6 | | mL | min |
| | | mmol | mmol | | | |
| Urea-homoLeu- | DMF | HATU | NMM | 50 min | 3% hydrazine | 30 |
| Tyr(tBu)-OH | (1.5 mL) | 0.285 | 0.6 | | hydrate/DMF 10 | min |
| 0.3 mmol | | mmol | mmol | | mL¹ | |
| DOTA- | DMF | HATU | NMM | 60 min | N/A | N/A |
| Tris(tBuester) | (1.5 mL) | 0.285 | 0.6 | | | |
| 0.3 mmol | | mmol | mmol | | | |

After the peptidyl resin was built up, it was washed with DCM (3×10 mL) and MeOH (3*10 mL). The resin was dried under vacuum overnight.

3% hydrazine hydrate in DMF (3 equivalent volume of the resin) was added into the peptidyl resin and the suspension was kept at room temperature while a stream of nitrogen bubbled through it. After 30 minutes, the suspension was filtered and the resin was washed with DMF (3 equivalent volume of the resin) for 5 times.

### Cleavage

Cleavage E solution (TFA:Thioanisole:phenol:EDT:H₂O=87.5:5:2.5:2.5:2.5, 10 mL) was added into peptidyl resin (0.758 g) under the protection of nitrogen. The mixture was shaken for 2.5 hours at room temperature. Cold ether (60 mL) was added into the filtrate, the peptide was precipitated by centrifugation (4000 revolutions/minute). The white precipitation was washed with ether (60 mL) twice. Then the crude was dried under vacuum overnight to give the crude as a white solid (0.091 g, purity: 10%).

### Oxidation

0.091 g crude was dissolved by 5 mL ACN, 10 mL H₂O and 10 mL AcOH. Then 1.5 mL of 12 in methanol (10 mg/mL) was added into the mixture and the mixture was stirred for 5 minutes at RT. Ascorbic acid was added into the solution with slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B of 39% to 69% over 60 minutes at a flow rate of 15 mL/minute (solvent A: 4 g/L Ammonium acetate in water; solvent B: 4 g/L Ammonium acetate in 70% ACN/H₂O). The product buffer with purity more than 74% was collected.

### Step 2

The buffer from step 1 with purity more than 74% was purified further by using a Hanbon 300 connected to Phenomenex Luna™ C18 100 Å 10 μm; 21*250 mm HPLC column. Method: gradient window of solvent B of 36% to 66% over 60 minutes at a flow rate of 15 mL/minute (solvent A: 0.1% TFA in water; solvent B: 0.1% TFA in 80% ACN/H₂O). The solution was collected and lyophilized to give the final peptide (3.5 mg, 91.7%) as a white solid.

### General Procedure U

### Example Cpd #477

CTC Resin (0.5 g, Sub: 0.97 mmol/g) was swelled in DMF (10 mL)₂ hours, Fmoc-DLys(Dde)-OH (0.106 g, 0.2 mmol) and DIPEA (0.42 mL, 2.5 mmol) were added into the suspension. The mixture was kept at room temperature for 3 hours while a stream of nitrogen bubbled through it. Then 1 mL MeOH was added into the mixture and resulting mixture was kept at room temperature for 0.5 hours. Reaction mixture was filtered. The resin was washed with DMF (3*10 mL) and then MeOH (3*10 mL).

The resin was swelled in DMF (10 mL) for 2 hours, then 20% piperidine in DMF (10 mL) was added into the resin. The mixture was kept at room temperature for 0.5 hours while a stream of nitrogen bubbled through it. The mixture was filtered and the peptidyl resin was washed with DMF (5*10 mL). Fmoc-Val(3-OH)—OH (0.2 mmol, 0.07 g), DMF (1.5 mL), NMM (0.4 mmol, 0.044 mL) and HATU (0.19 mmol, 0.072 g) were added into the resin sequentially. The suspension was kept at room temperature for 1 hour while a stream of nitrogen bubbled through it. The ninhydrine test indicated the coupling completed. The mixture was filtered and the peptidyl resin was washed with DMF (3*10 mL). 20% piperidine in DMF (10 mL) was added into the resin to remove the Fmoc group. The following amino acids were coupled to the resin bound peptide sequentially as follows:

Synthesis Table

| | Reaction | | | Reaction | | |
|---|---|---|---|---|---|---|
| Amino Acid | Solvent | Reagents | | Time | Deprotection reagents | |
| Fmoc-Phe-OH | DMF | HBTU | NMM | 75 min | 20% | 20 min |
| 0.3 mmol | (1.5 mL) | 0.285 | 0.6 | | Pip/DMF 10 | |
| | | mmol | mmol | | mL | |
| Fmoc-Trp(5-F)-OH | DMF | HATU | NMM | 60 min | 20% | 20 min |
| 0.2 mmol | (1.5 mL) | 0.19 | 0.4 | | Pip/DMF 10 | |
| | | mmol | mmol | | mL | |
| Fmoc-Cys(Trt)-OH | DMF | HBTU | NMM | 70 min | 20% | 30 min |
| 0.3 mmol | (1.5 mL) | 0.285 | 0.6 | | Pip/DMF 10 | |
| | | mmol | mmol | | mL | |
| Fmoc-Tyr(tBu)-OH | DMF | HBTU | NMM | 60 min | 20% | 30 min |
| 0.3 mmol | (1.5 mL) | 0.285 | 0.6 | | Pip/DMF 10 | |
| | | mmol | mmol | | mL | |
| Fmoc-Val-OH | DMF | HBTU | NMM | 50 min | 20% | 30 min |
| 0.3 mmol | (1.5 mL) | 0.285 | 0.6 | | Pip/DMF 10 | |
| | | mmol | mmol | | mL | |
| Fmoc-Asp(OtBu)- | DMF | HBTU | NMM | 55 min | 20% | 30 min |
| OH | (1.5 mL) | 0.285 | 0.6 | | Pip/DMF 10 | |
| 0.3 mmol | | mmol | mmol | | mL | |
| Fmoc-L- | DMF | HATU | NMM | 50 min | 20% | 30 min |
| Ala(tetrahydropyran- | (1.5 mL) | 0.19 | 0.4 | | Pip/DMF 10 | |
| 4-yl)-OH | | mmol | mmol | | mL | |
| 0.2 mmol | | | | | | |
| Fmoc-(1-Boc- | DMF | HATU | NMM | 50 min | 20% | 30 min |
| piperidin-4-yl)Ala- | (1.5 mL) | 0.19 | 0.4 | | Pip/DMF 10 | |
| OH | | mmol | mmol | | mL | |
| 0.2 mmol | | | | | | |
| Fmoc-Gly-OH | DMF | HBTU | NMM | 50 min | 20% | 30 min |
| 0.3 mmol | (1.5 mL) | 0.285 | 0.6 | | Pip/DMF 10 | |
| | | mmol | mmol | | mL | |
| Fmoc-Tbg-OH | DMF | HATU | NMM | 60 min | 20% | 30 min |
| 0.3 mmol | (1.5 mL) | 0.285 | 0.6 | | Pip/DMF 10 | |
| | | mmol | mmol | | mL | |
| Fmoc-Cys(Trt)-OH | DMF | HBTU | NMM | 60 min | 20% | 20 min |
| 0.3 mmol | (1.5 mL) | 0.285 | 0.6 | | Pip/DMF 10 | |
| | | mmol | mmol | | mL | |
| Fmoc-Tyr(tBu)-OH | DMF | HBTU | NMM | 45 min | 20% | 20 min |
| | | 0.285 | 0.6 | | Pip/DMF 10 | |
| 0.3 mmol | (1.5 mL) | mmol | mmol | | mL | |
| Fmoc-homoLeu- OH | DMF | HATU | NMM | 50 min | 20% | 30 min |
| 0.3 mmol | (1.5 mL) | 0.285 | 0.6 | | Pip/DMF 10 | |
| | | mmol | mmol | | mL | |
| Pyr | DMF | HATU | NMM | 60 min | 3% | 30 min |
| 0.4 mmol | (1.5 mL) | 0.38 | 0.6 | | hydrazine | |
| | | mmol | mmol | | hydrate/DMF | |
| | | | | | 10 mL¹ | |
| DOTA- | DMF | HATU | NMM | 60 min | N/A | N/A |
| Tris(tBuester) | (1.5 mL) | 0.285 | 0.6 | | | |
| 0.3 mmol | | mmol | mmol | | | |
| DOTA- | DMF | HATU | NMM | 60 min | N/A | N/A |
| Tris(tBuester) | (1.5 mL) | 0.285 | 0.6 | | | |
| 0.3 mmol | | mmol | mmol | | | |

After the peptidyl resin was built up, it was washed with DCM (3×10 mL) and MeOH (3*10 mL). The resin was dried under vacuum overnight.

3% hydrazine hydrate in DMF (3 equivalent volume of the resin) was added into the peptidyl resin and the suspension was kept at room temperature while a stream of nitrogen bubbled through it. After 30 minutes, the suspension was filtered and the resin was washed with DMF (3 equivalent volume of the resin) for 5 times.

### Cleavage

Cleavage E solution (TFA:Thioanisole:phenol:EDT:H₂O=87.5:5:2.5:2.5:2.5, 10 mL) was added into peptidyl resin (1.011 g) under the protection of nitrogen. The mixture was shaken for 2.5 hours at room temperature. Cold ether (60 mL) was added into the filtrate, the peptide was precipitated by centrifugation (4000 revolutions/minute). The white precipitation was washed with ether (60 mL) twice. Then the crude was dried under vacuum overnight to give the crude as a white solid (0.141 g, purity: 44.9%).

### Oxidation

0.141 g crude was dissolved by 5 mL ACN, 10 mL H₂O and 15 mL AcOH. Then 2.5 mL of 12 in methanol (10 mg/mL) was added into the mixture and the mixture was stirred for 5 minutes at RT. Ascorbic acid was added into the solution with slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B from 37% to 67% over 60 minutes at a flow rate of 15 mL/minute (solvent A: 4 g/L Ammonium acetate in water; solvent B: 4 g/L Ammonium acetate in 70% ACN/H₂O). The product buffer with purity more than 90% was collected.

### Step 2

The buffer from step 1 with purity more than 90% was purified further by using a Hanbon 300 connected to Phenomenex Luna™ C18 100 Å 10 μm; 21*250 mm HPLC column. Method: gradient window of solvent B from 38% to 68% over 60 minutes at a flow rate of 15 mL/minute (solvent A: 0.1% TFA in water; solvent B: 0.1% TFA in 80% ACN/H₂O). The solution was collected and lyophilized to give the final peptide (30.5 mg, 96.6%) as a white solid.

### General Procedure V

### Example Cpd #437

The Fmoc-DLys(Dde)-CTC Resin (0.1 mmol, 0.5 g; Sub: 0.2 mmol/g) was swelled in DMF (10 mL) for 2 hours, then 20% piperidine in DMF (10 mL) was added into the resin. The mixture was kept at room temperature for 0.5 hours while a stream of nitrogen bubbled through it. The mixture was filtered and the peptidyl resin was washed with DMF (5*10 mL). Fmoc-Val(3-OH)—OH (0.2 mmol, 0.071 g), DMF (1.5 mL), NMM (0.4 mmol, 0.044 mL) and HATU (0.19 mmol, 0.072 g) were added into the resin sequentially. The suspension was kept at room temperature for 1 hour while a stream of nitrogen bubbled through it. The ninhydrine test indicated the coupling completed. The mixture was filtered and the peptidyl resin was washed with DMF (3*10 mL). 20% piperidine in DMF (10 mL) was added into the resin to remove the Fmoc group. The following amino acids were coupled to the resin bound peptide sequentially as follows:

Synthesis Table

| | Reaction | | | Reaction | | |
|---|---|---|---|---|---|---|
| Amino Acid | Solvent | Reagents | | Time | Deprotection reagents | |
| Fmoc-Phe-OH | DMF | HBTU | NMM | 46 min | 20% Pip/DMF 10 | 30 min |
| 0.3 mmol | (1.5 mL) | 0.285 | 0.6 | | mL | |
| | | mmol | mmol | | | |
| Fmoc-Trp(5-F)-OH | DMF | HATU | NMM | 48 min | 20% Pip/DMF 10 | 21 min |
| 0.2 mmol | (1.5 mL) | 0.19 | 0.4 | | mL | |
| | | mmol | mmol | | | |
| Fmoc-Cys(Trt)-OH | DMF | HBTU | NMM | 50 min | 20% Pip/DMF 10 | 30 min |
| 0.3 mmol | (1.5 mL) | 0.285 | 0.6 | | mL | |
| | | mmol | mmol | | | |
| Fmoc-Tyr(tBu)-OH | DMF | HBTU | NMM | 63 min | 20% Pip/DMF 10 | 25 min |
| 0.3 mmol | (1.5 mL) | 0.285 | 0.6 | | mL | |
| | | mmol | mmol | | | |
| Fmoc-Val-OH | DMF | HBTU | NMM | 60 min | 20% Pip/DMF 10 | 20 min |
| 0.3 mmol | (1.5 mL) | 0.285 | 0.6 | | mL | |
| | | mmol | mmol | | | |
| Fmoc-Asp(OtBu)- | DMF | HBTU | NMM | 60 min | 20% Pip/DMF 10 | 27 min |
| OH | (1.5 mL) | 0.285 | 0.6 | | mL | |
| 0.3 mmol | | mmol | mmol | | | |
| Fmoc-L- | DMF | HATU | NMM | 53 min | 20% Pip/DMF 10 | 30 min |
| Ala(tetrahydropyran- | (1.5 mL) | 0.19 | 0.4 | | mL | |
| 4-yl)-OH | | mmol | mmol | | | |
| 0.2 mmol | | | | | | |
| Fmoc-(1-Boc- | DMF | HATU | NMM | 85 min | 20% Pip/DMF 10 | 25 min |
| piperidin-4-yl)Ala- | (1.5 mL) | 0.19 | 0.4 | | mL | |
| OH | | mmol | mmol | | | |
| 0.2 mmol | | | | | | |
| Fmoc-Gly-OH | DMF | HBTU | NMM | 70 min | 20% Pip/DMF 10 | 20 min |
| 0.3 mmol | (1.5 mL) | 0.285 | 0.6 | | mL | |
| | | mmol | mmol | | | |
| Fmoc-Tbg-OH | DMF | HATU | NMM | 60 min | 20% Pip/DMF 10 | 20 min |
| 0.3 mmol | (1.5 mL) | 0.285 | 0.6 | | mL | |
| | | mmol | mmol | | | |
| Fmoc-Cys(Trt)-OH | DMF | HBTU | NMM | 45 min | 20% Pip/DMF 10 | 30 min |
| 0.3 mmol | (1.5 mL) | 0.285 | 0.6 | | mL | |
| | | mmol | mmol | | | |
| Fmoc-Tyr(tBu)-OH | DMF | HBTU | NMM | 60 min | 20% Pip/DMF 10 | 25 min |
| 0.3 mmol | (1.5 mL) | 0.285 | 0.6 | | mL | |
| | | mmol | mmol | | | |
| Fmoc-homoLeu-OH | DMF | HATU | NMM | 60 min | 20% Pip/DMF 10 | 31 min |
| 0.2 mmol | (1.5 mL) | 0.19 | 0.4 | | mL | |
| | | mmol | mmol | | | |
| Methanesulfonyl | DMF | N/A | DIPEA | 60 min | 3% hydrazine | 30 min |
| chloride | (1.5 mL) | N/A | 0.4 | | hydrate/DMF 10 | |
| 0.2 mmol | | | moll | | mL¹ | |
| DOTA- | DMF | HATU | NMM | 44 min | N/A | N/A |
| Tris(tBuester) | (1.5 mL) | 0.285 | 0.6 | | | |
| 0.3 mmol | | mmol | mmol | | | |

After the peptidyl resin was built up, it was washed with DCM (3×10 mL) and MeOH (3*10 mL). The resin was dried under vacuum overnight.

3% hydrazine hydrate in DMF (3 equivalent volume of the resin) was added into the peptidyl resin and the suspension was kept at room temperature while a stream of nitrogen bubbled through it. After 30 minutes, the suspension was filtered and the resin was washed with DMF (3 equivalent volume of the resin) for 5 times.

### Cleavage

Cleavage E solution (TFA:Thioanisole:phenol:EDT:H₂O=87.5:5:2.5:2.5:2.5, 10 mL) was added into peptidyl resin (0.8 g) under the protection of nitrogen. The mixture was shaken for 2.5 hours at room temperature. Cold ether (60 mL) was added into the filtrate, the peptide was precipitated by centrifugation (4000 revolutions/minute). The white precipitation was washed with ether (60 mL) twice. Then the crude was dried under vacuum overnight to give the crude as a white solid (0.130 g, purity: 17%).

### Oxidation

0.130 g crude was dissolved by 15 mL ACN, 15 mL H₂O and 15 mL AcOH. Then 2 mL of 12 in methanol (10 mg/mL) was added into the mixture and the mixture was stirred for 5 minutes at RT. Ascorbic acid was added into the solution with slow magnetic stirring until the solution changed to colorless. The solution was filtered with 0.45 μm filter.

### Purification

### Step 1

The filtrate was purified by using a Hanbon 300 connected to Phenomenex Luna™ C18 100 Å 10 μm; 21*250 mm HPLC column. Method: gradient window of solvent B from 41% to 71% over 60 minutes at a flow rate of 15 mL/minute (solvent A: 4 g/L Ammonium acetate in water; solvent B: 4 g/L Ammonium acetate in 70% ACN/H₂O). The product buffer with purity more than 90% was collected.

### Step 2

The buffer from step 1 with purity more than 90% was purified further by using a Hanbon 300 connected to Phenomenex Luna™ C18 100 Å 10 μm; 21*250 mm HPLC column. Method: gradient window of solvent B from 39% to 69% over 60 minutes at a flow rate of 15 mL/minute (solvent A: 0.1% TFA in water; solvent B: 0.1% TFA in 80% ACN/H₂O). The solution was collected and lyophilized to give the final peptide (15.8 mg, 95.6%) as a white solid.

### General Procedure W

### Example Cpd #6

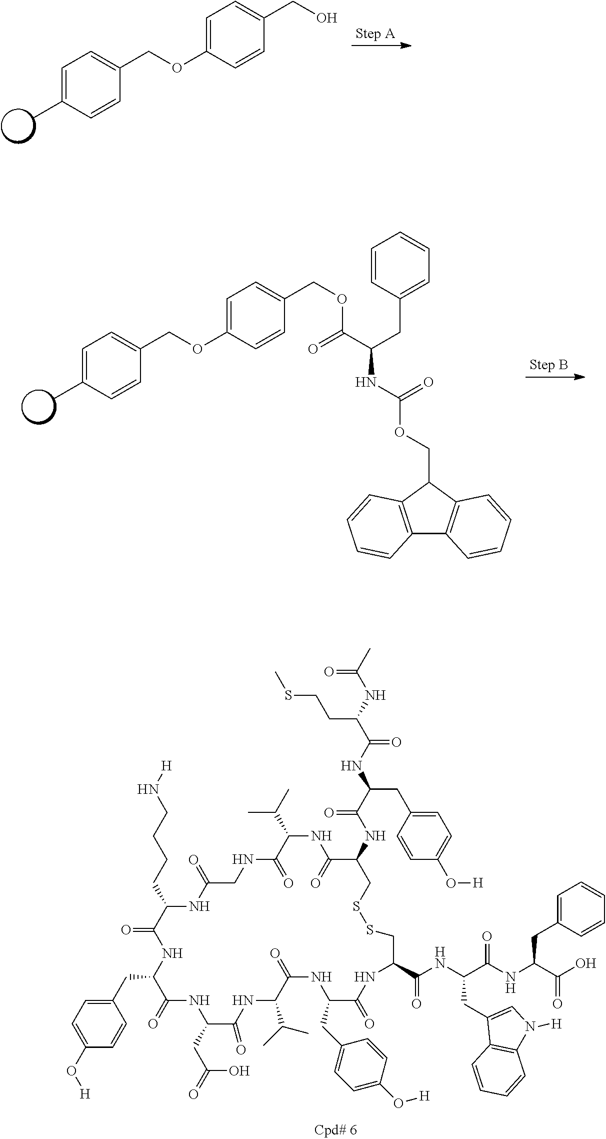 [SMILES: Cc1ccc(COc2ccc(CO)cc2)cc1 ; Cc1ccc(COc2ccc(COC(=O)[C@@H](Cc3ccccc3)NC(=O)OCC3c4ccccc4-c4ccccc43)cc2)cc1 ; CSCC[C@H](NC(C)=O)C(=O)N[C@@H](Cc1ccc(O)cc1)C(=O)N[C@H]1CSSC[C@@H](C(=O)N[C@@H](Cc2c[nH]c3ccccc23)C(=O)N[C@@H](Cc2ccccc2)C(=O)O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCN)NC(=O)CNC(=O)[C@H](C(C)C)NC1=O]

### Step a. Loading of C-Terminal Amino Acid on Wang Resin

Fmoc-Phe-OH (10 equiv.) was dissolved in a solution of dry DCM/dry DMF 10:1 v/v at 0.5 M concentration, under N₂ atmosphere. The solution was cooled at 0° C. in a ice batch and DIC (5 equiv.) was added. The resulting solution was stirred at 0° C. for 20 minutes. Then it was concentrated to dryness and re-dissolved in dry DMF at 0.5 M concentration and added to Wang resin (PS matrix, 100-200 mesh, Novabiochem, cat #8.55121, loading: 0.37 mmol/g) together with DMAP (0.1 equiv.). Resin was stirred at room temperature for 1 hour, then washed with DMF, MeOH and DCM, and the synthesis was continued on Cem Liberty Prime synthesizer.

### Step B. Synthesis of Compound Cpd #6

The peptide was synthesized by standard Solid-phase Peptide Synthesis (SPPS) using Fmoc/t-Bu chemistry. The assembly was performed on a 100 umol scale on the Cem Liberty Prime microwave peptide synthesizer (CEM Inc.).

All the amino acids were dissolved at a 0.5 M concentration in DMF. The acylation reactions were performed for 2 minutes at 90° C. under MW irradiation with 5 folds excess of activated amino acids over the resin free amino groups. The amino acids were activated with equimolar amounts of 2 M solution of DIC in DMF and Oxyma solution 0.25 M in DMF.

Double acylation reactions were performed for bulky residues (including alpha-methylated, N-methylated or beta-branched amino acids) and following amino acid residue after the bulky one.

N-terminal acetylation was performed for 2 minutes at 65° C. under MW irradiation with a 10% v/v solution of AC₂O in DMF.

The peptide was cleaved from solid support using 20 ml of TFA solution (v/v: 87.5% TFA, 5% H₂O, 2.5% TIPS, 5% Phenol) for approximately 4 hours, at room temperature. The resin was then filtered, and the solution concentrated to about 5 mL and precipitated in cold MTBE (45 mL). After centrifugation, the peptide pellets were washed with fresh cold Et₂O to remove the organic scavengers. The process was repeated twice. Final pellets were dried, re-suspended in H2O/ACN (1 mg/mL). Iodine (saturated solution in AcOH) was added until yellow color persisted. Stirred at room temperature for 5 minutes, then quenched with ascorbic acid and lyophilized.

Crude peptide was purified by reverse-phase HPLC using preparative Waters XBridge C18 column (250×50 mm, 130 Å, 5 μm). Mobile phase A: H₂O+0.1% TFA, mobile phase B: ACN+0.1% TFA. The following gradient of eluent B was used: 205 for 5 minutes, then to 45% over 20 minutes, flow rate 80 mL/minute, wavelength 214 nm. The resulting fractions were lyophilized to afford the desired product Cpd #6.

### General Procedure X

### Example Cpd #331

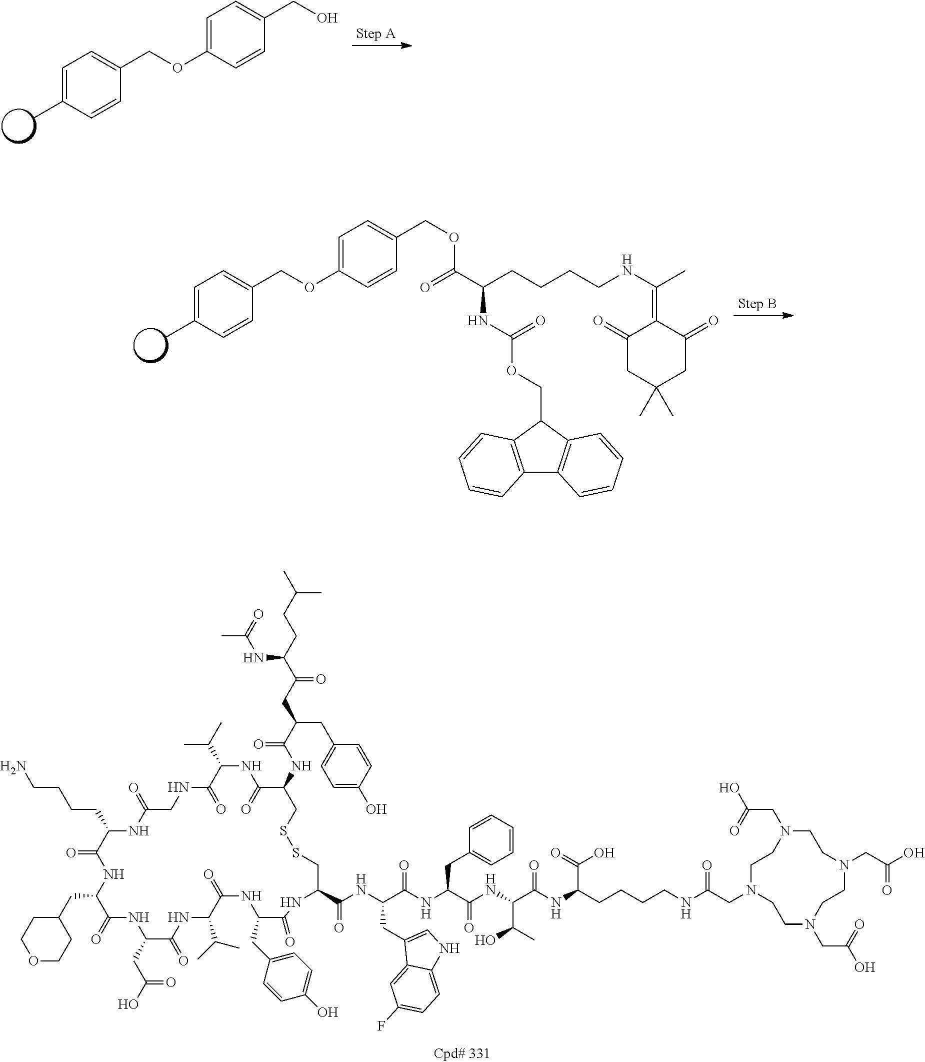 [SMILES: Cc1ccc(COc2ccc(CO)cc2)cc1 ; CC(NCCCC[C@@H](NC(=O)OCC1c2ccccc2-c2ccccc21)C(=O)OCc1ccc(OCc2ccc(C)cc2)cc1)=C1C(=O)CC(C)(C)CC1=O ; CC(=O)N[C@@H](CCC(C)C)C(=O)C[C@@H](Cc1ccc(O)cc1)C(=O)N[C@H]1CSSC[C@@H](C(=O)N[C@@H](Cc2c[nH]c3ccc(F)cc23)C(=O)N[C@@H](Cc2ccccc2)C(=O)N[C@H](C(=O)N[C@H](CCCCNC(=O)CN2CCN(CC(=O)O)CCN(CC(=O)O)CCN(CC(=O)O)CC2)C(=O)O)[C@@H](C)O)NC(=O)[C@H](Cc2ccc(O)cc2)NC(=O)[C@H](C(C)C)NC(=O)[C@H](CC(=O)O)NC(=O)[C@H](CC2CCOCC2)NC(=O)[C@H](CCCCN)NC(=O)CNC(=O)[C@H](C(C)C)NC1=O]

### Step a. Loading of C-Terminal Amino Acid on Wang Resin

Fmoc-D-Lys(Dde)-OH (10 equiv.) was dissolved in a solution of dry DCM/dry DMF 10:1 v/v at 0.5 M concentration, under N₂ atmosphere. The solution was cooled at 0° C. in a ice batch and DIC (5 equiv.) was added. The resulting solution was stirred at 0° C. for 20 minutes. Then it was concentrated to dryness and re-dissolved in dry DMF at 0.5 M concentration and added to Wang resin (PS matrix, 100-200 mesh, Novabiochem, cat #8.55121, loading: 0.37 mmol/g) together with DMAP (0.1 equiv.). Resin was stirred at room temperature for 1 hour, then washed with DMF, MeOH and DCM, and the synthesis was continued on Cem Liberty Prime synthesizer.

### Step B. Synthesis of Compound Cpd #331

The peptide was synthesized by standard Solid-phase Peptide Synthesis (SPPS) using Fmoc/t-Bu chemistry. The assembly was performed on a 100 umol scale on the Cem Liberty

Prime microwave peptide synthesizer (CEM Inc.).

All the amino acids were dissolved at a 0.5 M concentration in DMF. The acylation reactions were performed for 2 minutes at 90° C. under MW irradiation with 5 folds excess of activated amino acids over the resin free amino groups. The amino acids were activated with equimolar amounts of 2 M solution of DIC in DMF and Oxyma solution 0.25 M in DMF.

Double acylation reactions were performed for bulky residues (including alpha-methylated, N-methylated or beta-branched amino acids) and following amino acid residue after the bulky one.

N-terminal acetylation was performed for 2 minutes at 65° C. under MW irradiation with a 10% v/v solution of AC₂O in DMF.

At the end of the assembly, a 4% hydrazine monohydrate solution in DMF (20 mL) was percolated on the resin over 15 minutes to selectively remove the Dde protecting group from the D-Lys.

DOTA was incorporated manually using an equimolar solution of DOTA(tBu)3, DIC and HOBt (2 equiv., 1:1:1) in NMP at room temperature and complete acylation was monitored by ninhydrin test.

The peptide was cleaved from solid support using 20 ml of TFA solution (v/v: 87.5% TFA, 5% H₂O, 2.5% TIPS, 5% Phenol) for approximately 4 hours, at room temperature. The resin was then filtered, and the solution concentrated to about 5 mL and precipitated in cold MTBE (45 mL). After centrifugation, the peptide pellets were washed with fresh cold Et₂O to remove the organic scavengers. The process was repeated twice. Final pellets were dried, re-suspended in H₂O/ACN (1 mg/mL). Iodine (saturated solution in AcOH) was added until yellow color persisted. Stirred at room temperature for 5 minutes, then quenched with ascorbic acid and lyophilized.

Crude peptide was purified by reverse-phase HPLC using preparative Waters XBridge C18 column (250×50 mm, 130 Å, 5 μm). Mobile phase A: H₂O+0.1% TFA, mobile phase B: ACN+0.1% TFA. The following gradient of eluent B was used: 25% for 5 minutes, then to 40% over 20 minutes, flow rate 80 mL/minute, wavelength 214 nm. The resulting fractions were lyophilized to afford the desired product Cpd #331.

### General Procedure Z

### Example Cpd #329

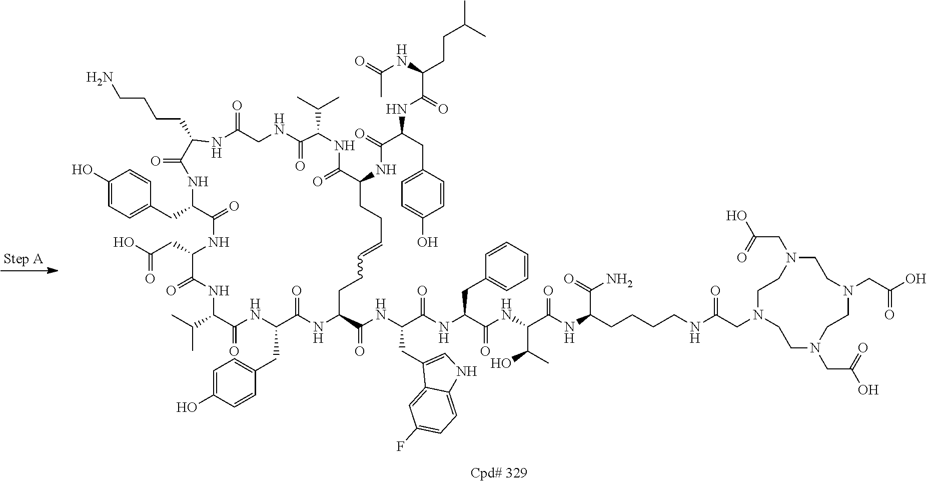 [SMILES: CC(=O)N[C@@H](CCC(C)C)C(=O)N[C@@H](Cc1ccc(O)cc1)C(=O)N[C@H]1CCC=CCC[C@@H](C(=O)N[C@@H](Cc2c[nH]c3ccc(F)cc23)C(=O)N[C@@H](Cc2ccccc2)C(=O)N[C@H](C(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCN)NC(=O)CNC(=O)[C@H](C(C)C)NC1=O]

### Step A. Synthesis of Cpd #329

The peptide was synthesized by standard Solid-phase Peptide Synthesis (SPPS) using Fmoc/t-Bu chemistry. The assembly was performed on a Rink-amide MBHA resin (250 umol, 100-200 Mesh; loading 0.42 mmol/g) on the Cem Liberty Blue microwave peptide synthesizer (CEM Inc.). During peptide assembly on solid phase, the side chain of D-Lys was protected with Dde.

All the amino acids were dissolved at a 0.2 M concentration in DMF. The acylation reactions were performed for 2 minutes at 90° C. under MW irradiation with 4 folds excess of activated amino acids over the resin free amino groups. The amino acids were activated with equimolar amounts of 1 M solution of DIC in DMF and 1 M Oxyma solution in DMF.

Double acylation reactions were performed for bulky residues (including alpha-methylated, N-methylated or beta-branched amino acids) and following amino acid residue after the bulky one.

N-terminal acetylation was performed for 2 minutes at 65° C. under MW irradiation with a 10% v/v solution of AC₂O in DMF.

At the end of the assembly the resin was washed with DMF, DCM, Et₂O.

Resin-loaded peptide (100 umol) was suspended in dry DMF (10 mL) under N₂ atmosphere. Grubbs2 catalyst (0.5 equiv.) was added. Stirred at 110° C. in oil bath for 1 hour, then the resin was washed with DMF, DCM, Et₂O and the process was repeated. Reaction monitored by test cleavage.

After RCM reaction, a 4% hydrazine monohydrate solution in DMF (20 mL) was percolated on the resin over 15 minutes to selectively remove the Dde protecting group from the D-Lys.

DOTA was incorporated manually using an equimolar solution of DOTA(tBu)3, DIC and HOBt (2 equiv., 1:1:1) in NMP at room temperature and complete acylation was monitored by ninhydrin test.

The peptide was cleaved from solid support using 20 ml of TFA solution (v/v: 87.5% TFA, 5% H₂O, 2.5% TIPS, 5% Phenol) for approximately 4 hours, at room temperature. The resin was then filtered, and the solution concentrated to about 4 mL and precipitated in cold MTBE (45 mL). After centrifugation, the peptide pellets were washed with fresh cold Et₂O to remove the organic scavengers. The process was repeated twice. Final pellets were dried, re-suspended in H₂O and ACN 1:1 and purified by reverse-phase HPLC using preparative Waters XBridge C18 column (250×50 mm, 130 Å, 5 μm). Mobile phase A: H₂O+0.1% TFA, mobile phase B: ACN+0.1% TFA. The following gradient of eluent B was used: 25% for 5 minutes, then to 40% over 20 minutes, flow rate 80 mL/minute, wavelength 214 nm. Collected fractions were lyophilized to afford Compound Cpd #329. The configuration of cis/trans geometry of double bond was not verified.

### General Procedure Z2

### Example Cpd #241

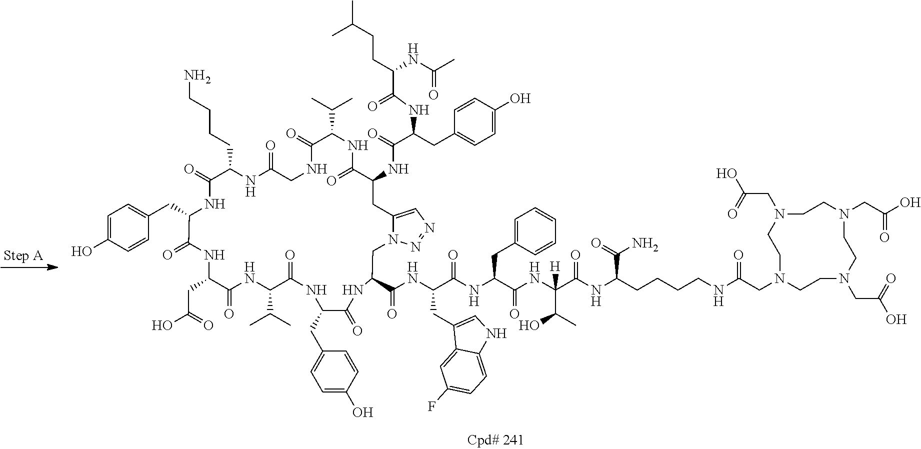 [SMILES: CC(=O)N[C@@H](CCC(C)C)C(=O)N[C@@H](Cc1ccc(O)cc1)C(=O)N[C@H]1Cc2cnnn2C[C@@H](C(=O)N[C@@H](Cc2c[nH]c3ccc(F)cc23)C(=O)N[C@@H](Cc2ccccc2)C(=O)N[C@H](C(=O)N[C@H](CCCCNC(=O)CN2CCN(CC(=O)O)CCN(CC(=O)O)CCN(CC(=O)O)CC2)C(N)=O)[C@@H](C)O)NC(=O)[C@H](Cc2ccc(O)cc2)NC(=O)[C@H](C(C)C)NC(=O)[C@H](CC(=O)O)NC(=O)[C@H](Cc2ccc(O)cc2)NC(=O)[C@H](CCCCN)NC(=O)CNC(=O)[C@H](C(C)C)NC1=O]

### Step A. Synthesis of Compound Cpd #241

The peptide was synthesized by standard Solid-phase Peptide Synthesis (SPPS) using Fmoc/t-Bu chemistry. The assembly was performed on a PS Rink-amide MBHA resin (250 umol, 100-200 Mesh; loading 0.42 mmol/g) on the Cem Liberty Blue microwave peptide synthesizer (CEM Inc.).

All the amino acids were dissolved at a 0.2 M concentration in DMF. The acylation reactions were performed for 2 minutes at 90° C. under MW irradiation with 4 folds excess of activated amino acids over the resin free amino groups. The amino acids were activated with equimolar amounts of 1 M solution of DIC in DMF and Oxyma solution 1 M in DMF. The Fmoc deprotection reactions were performed for 3 minutes at 90° C. using a 20% piperidine solution in DMF.

Double acylation reactions were performed for bulky residues (including alpha-methylated, N-methylated or beta-branched amino acids) and following amino acid residue after the bulky one.

N-terminal acetylation was performed for 2 minutes at 65° C. under MW irradiation with a 10% v/v solution of AC₂O in DMF.

1,5-Triazole formation was performed by suspending the resin-bound peptide (190 umol) in a solution of Pentamethylcyclopentadienylbis (triphenylphosphine) ruthenium (II) chloride (0.25 equiv.) in DMF (7 mL). Stirred for 2 hours at 100° C. under MW irradiation.

Dde protecting group was removed from D-Lys side chain by treating the resin with a 4% solution of hydrazine monohydrate in DMF (40 mL) over 30 minutes at room temperature. DOTA was incorporated by manual coupling using a mixture of DOTA(tBu)₃ (3 equiv.), HATU (3 equiv.) and DIPEA (6 equiv.) in DMF, for 1 hour at room temperature.

At the end of the assembly the resin was washed with DMF, DCM, Et₂O. The resin bound peptide was cleaved from solid support using 40 ml of TFA solution (v/v: 87.5% TFA, 5% H₂O, 2.5% TIPS, 5% Phenol) for approximately 4 hours, at room temperature. The resin was then filtered and concentrated to about 5 mL and precipitated in cold MTBE (40 mL). After centrifugation, the peptide pellets were washed with fresh cold Et₂O to remove the organic scavengers. The process was repeated twice. Final pellets were dried, re-suspended in H₂O and ACN 1:1 and purified by reverse-phase HPLC in two runs using preparative Waters XBridge C18 column (250×50 mm, 130 Å, 5 μm). Mobile phase A: H₂O+0.1% TFA, mobile phase B: ACN+0.1% TFA. The following gradient of eluent B was used: 25% for 5 minutes, then to 40% over 20 minutes, flow rate 80 mL/minute, wavelength 214 nm. Collected fractions were lyophilized to afford pure Compound Cpd #241.

### General Procedure Z3

### Example Cpd #242

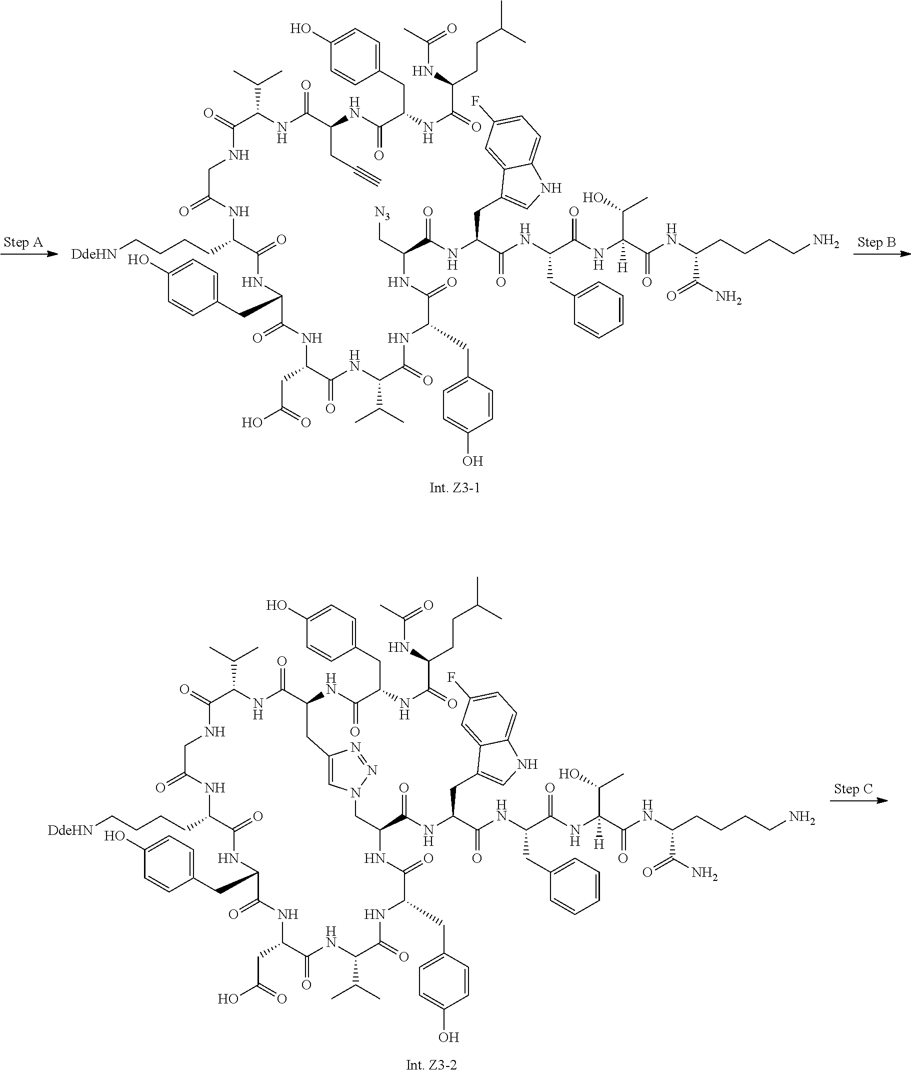

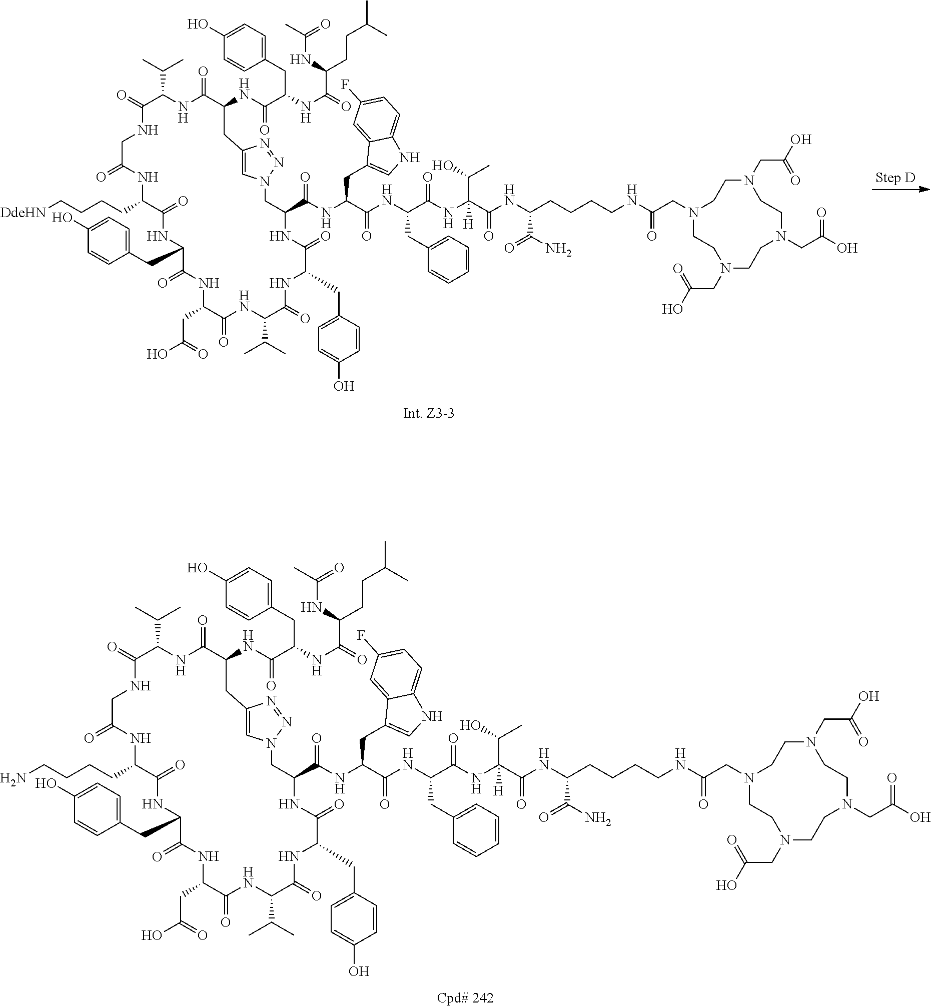

Step A. Synthesis of Compound Int. Z3-1

The peptide was synthesized by standard Solid-phase Peptide Synthesis (SPPS) using Fmoc/t-Bu chemistry. The assembly was performed on a PS Rink-amide MBHA resin (250 umol, 100-200 Mesh; loading 0.42 mmol/g) on a Liberty Blue microwave peptide synthesizer (CEM Inc.). During peptide assembly on solid phase, the side chain protecting groups were: Dde for Lys and Boc for D-Lys.

All the amino acids were dissolved at a 0.2 M concentration in DMF. The amino acids were activated with equimolar amounts of a 1 M solution of DIC in DMF and a 1 M solution of Oxyma in DMF. The acylation reactions were performed for 2 minutes at 90° C. under MW irradiation with 4 folds excess of activated amino acids over the resin free amino groups. The Fmoc deprotection reactions were performed for 3 minutes at 90° C. using a 20% piperidine solution in DMF.

Double acylation reactions were performed for bulky residues (including alpha-methylated, N-methylated or beta-branched amino acids) and following amino acid residue after the bulky one.

N-terminal acetylation was performed for 2 minutes at 65° C. under MW irradiation with a 10% v/v solution of AC₂O in DMF.

At the end of the assembly the resin was washed with DMF, DCM, Et₂O. The resin bound peptide was cleaved from solid support using 15 mL of TFA solution (v/v: 87.5% TFA, 5% H₂O, 2.5% TIPS, 5% Phenol) for approximately 4 hours, at room temperature. The resin was then filtered and concentrated to about 5 mL and precipitated in cold MTBE (40 mL). After centrifugation, the peptide pellets were washed with fresh cold Et₂O to remove the organic scavengers. The process was repeated twice. Final pellets were dried, re-suspended in H₂O and ACN 1:1 and lyophilized to afford crude Compound Int. Z3-1.

### Step B. Synthesis of Compound Int. Z3-2

Intermediate Compound Int. Z3-1 was dissolved in a mixture of H2O/DMF (1:1, 1 mg/mL). CuSO₄·5H₂O (3 equiv.) and sodium ascorbate (5 equiv.) were added and stirred at room temperature for 5 minutes, then quenched with TFA, concentrated to dryness and lyophilized. Crude peptide was purified by reverse-phase HPLC using a preparative Waters XBridge C18 column (250×50 mm, 130 Å, 5 μm) with mobile phase A: H₂O+0.1% TFA, mobile phase B: ACN+0.1% TFA. The following gradient of eluent B was used: 25% for 5 minutes, to 45% over 20 minutes, flow rate 80 mL/minute, and UV detection at wavelength 214 nm. Collected fractions were lyophilized to afford Compound Int. Z3-2.

### Step C. Synthesis of Compound Int. Z3-3

A solution of DOTA(tBu)3, (1.3 equiv.), HATU (1.1 equiv.) and DIPEA (4 equiv.) in DMF at 0.05 M concentration was stirred for 10 minutes at room temperature. Then pure Compound Int. Z3-2 (1 equiv.) was added and stirred for 2 hours at room temperature, then quenched with acetic acid and concentrated to dryness. Crude peptide was stirred in 20 mL of TFA solution (v/v: 87.5% TFA, 10% HCl 6N, 2.5% TIPS) for 10 minutes, at room temperature, then concentrated to dryness and lyophilized to afford the crude intermediate Int. Z3-3.

### Step D. Synthesis of Compound Cpd #242

Dde protecting group was removed from Lys side chain by treating the resin with a 4% solution of hydrazine monohydrate in DMF (40 mL) over 30 minutes at room temperature. Crude peptide was purified by reverse-phase HPLC using a preparative Waters XBridge C18 column (250×50 mm, 130 Å, 5 μm) and mobile phase A: H₂O+0.1% TFA, mobile phase B: ACN+0.1% TFA. The following gradient of eluent B was used: 25% for 5 minutes, to 40% over 20 minutes, flow rate 80 mL/minute, and UV detection wavelength 214 nm. Collected fractions were lyophilized to afford Cpd #242.

Tables 5A and 5B below shows the synthetic procedure that was used to synthesize the compounds described herein.

TABLE 5

| Cpd # | Synthetic procedure |
|---|---|
| 1 | J |
| 2 | A |
| 3 | D |
| 4 | A |
| 5 | D |
| 6 | W |
| 7 | J |
| 8 | J |
| 9 | J |
| 10 | J |
| 11 | J |
| 12 | J |
| 13 | J |
| 14 | J |
| 15 | J |
| 16 | J |
| 17 | J |
| 18 | J |
| 19 | J |
| 20 | J |
| 21 | J |
| 22 | J |
| 23 | J |
| 24 | J |
| 25 | J |
| 26 | J |
| 27 | K |
| 28 | X |
| 29 | K |
| 30 | K |
| 31 | K |
| 32 | J |
| 33 | W |
| 34 | J |
| 35 | J |
| 36 | J |
| 37 | J |
| 38 | J |
| 39 | J |
| 40 | J |
| 41 | J |
| 42 | J |
| 43 | J |
| 44 | J |
| 45 | J |
| 46 | J |
| 47 | J |
| 48 | J |
| 49 | J |
| 50 | K |
| 51 | W |
| 52 | J |
| 53 | J |
| 54 | K |
| 55 | K |
| 56 | K |
| 57 | K |
| 58 | K |
| 59 | J |
| 60 | J |
| 61 | K |
| 62 | K |
| 63 | K |
| 64 | K |
| 65 | K |
| 66 | K |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | K |
| 80 | K |
| 81 | K |
| 82 | K |
| 83 | K |
| 84 | K |
| 85 | K |
| 86 | K |
| 87 | K |
| 88 | K |
| 89 | K |
| 90 | K |
| 91 | K |
| 92 | K |
| 93 | K |
| 94 | J |
| 95 | A |
| 96 | A |
| 97 | A |
| 98 | A |
| 99 | A |
| 101 | M |
| 102 | M |
| 103 | M |
| 104 | M |
| 105 | M |
| 106 | M |
| 107 | M |
| 108 | M |
| 109 | M |
| 110 | M |
| 111 | M |
| 112 | M |
| 113 | M |
| 114 | M |
| 115 | M |
| 116 | M |
| 117 | A |
| 118 | A |
| 119 | A |
| 120 | A |
| 121 | A |
| 122 | A |
| 123 | A |
| 124 | A |
| 125 | A |
| 126 | A |
| 127 | A |
| 128 | K |
| 129 | K |
| 130 | L |
| 131 | K |
| 132 | K |
| 133 | K |
| 134 | K |
| 135 | K |
| 137 | K2 |
| 138 | A |
| 139 | A |
| 140 | A |
| 141 | A |
| 146 | A |
| 147 | A |
| 148 | A |
| 149 | K |
| 150 | K |
| 151 | K |
| 152 | K |
| 153 | K |
| 154 | K |
| 155 | K |
| 156 | K |
| 157 | K |
| 158 | K |
| 159 | K |
| 160 | A |
| 161 | A |
| 162 | A |
| 163 | A |
| 164 | A |
| 165 | A |
| 166 | A |
| 167 | A |
| 168 | A |
| 170 | A |
| 171 | A |
| 172 | A |
| 173 | A |
| 174 | A |
| 175 | A |
| 176 | A |
| 177 | A |
| 178 | A |
| 179 | A |
| 180 | A |
| 181 | A |
| 184 | K |
| 185 | K |
| 186 | K |
| 187 | L |
| 188 | L |
| 189 | L |
| 190 | K |
| 192 | A |
| 193 | A |
| 194 | A |
| 195 | A |
| 196 | A |
| 197 | A |
| 198 | A |
| 199 | A |
| 200 | A |
| 204 | A |
| 205 | A |
| 206 | A |
| 207 | A |
| 208 | A |
| 209 | A |
| 210 | A |
| 211 | A |
| 212 | A |
| 213 | A |
| 214 | K2 |
| 215 | K |
| 216 | K |
| 217 | K |
| 218 | K2 |
| 219 | A |
| 220 | A |
| 221 | A |
| 222 | A |
| 223 | A |
| 224 | A |
| 225 | A |
| 226 | A |
| 227 | A |
| 231 | A |
| 234 | A |
| 235 | A |
| 236 | A |
| 237 | K |
| 238 | L |
| 239 | L |
| 240 | K |
| 241 | Z3 |
| 242 | Z3 |
| 243 | A |
| 244 | A |
| 245 | A |
| 246 | A |
| 247 | A |
| 248 | A |
| 259 | A |
| 271 | A |
| 272 | D |
| 274 | A |
| 275 | A |
| 276 | A |
| 277 | A |
| 278 | A |
| 280 | A |
| 281 | A |
| 282 | A |
| 283 | D |
| 284 | D |
| 285 | D |
| 286 | A |
| 287 | A |
| 288 | A |
| 291 | A |
| 293 | D |
| 294 | D |
| 297 | L |
| 298 | L |
| 299 | L |
| 300 | L |
| 303 | B |
| 304 | B |
| 306 | E |
| 307 | B |
| 308 | B |
| 310 | B |
| 311 | B |
| 312 | B |
| 313 | B |
| 314 | A |
| 315 | A |
| 318 | B |
| 319 | B |
| 320 | A |
| 321 | B |
| 327 | B |
| 329 | Z |
| 331 | X |
| 332 | X |
| 333 | X |
| 334 | X |
| 335 | X |
| 339 | B |
| 340 | B |
| 341 | G |
| 345 | X |
| 347 | X |
| 348 | X |
| 349 | X |
| 350 | X |
| 351 | X |
| 352 | X |
| 353 | X |
| 354 | X |
| 355 | X |
| 356 | X |
| 357 | X |
| 358 | X |
| 359 | X |
| 360 | X |
| 361 | X |
| 362 | X |
| 363 | G |
| 364 | C* |
| 365 | B |
| 366 | A |
| 367 | B |
| 368 | B |
| 369 | B |
| 370 | B |
| 371 | M |
| 372 | M |
| 373 | M |
| 374 | M |
| 375 | X |
| 376 | X |
| 377 | X |
| 378 | X |
| 379 | X |
| 380 | X |
| 381 | X |
| 386 | M |
| 389 | P |
| 390 | P |
| 391 | O |
| 392 | O |
| 393 | O |
| 394 | O |
| 395 | O |
| 396 | B |
| 397 | B |
| 398 | B |
| 399 | X |
| 400 | X |
| 401 | X |
| 402 | X |
| 403 | X |
| 404 | J |
| 405 | G |
| 427 | M |
| 428 | M |
| 429 | M |
| 430 | M |
| 431 | M |
| 432 | M |
| 433 | M |
| 434 | M |
| 435 | M |
| 436 | M |
| 437 | V |
| 438 | M |
| 439 | M |
| 440 | B |
| 441 | B |
| 442 | B |
| 443 | B |
| 450 | B |
| 451 | B |
| 452 | B |
| 456 | M |
| 457 | M |
| 461 | M |
| 462 | M |
| 463 | M |
| 464 | B |
| 465 | B |
| 467 | M |
| 468 | T |
| 469 | M |
| 470 | M |
| 471 | M |
| 472 | M |
| 473 | M |
| 477 | U |
| 478 | P |
| 479 | B |
| 480 | B |
| 481 | B |
| 482 | I |
| 491 | M |
| 492 | M |
| 493 | M |
| 497 | B |
| 498 | B |
| 499 | B |
| 500 | B |
| 501 | B |
| 502 | B |
| 503 | B |
| 504 | U |
| 505 | U |
| 506 | M |
| 507 | U |
| 508 | B |
| 509 | M |
| 515 | C |
| 516 | B |
| 517 | S |
| 524 | B |
| 525 | B |
| 527 | M |
| 528 | M |
| 529 | M |
| 530 | M |
| 531 | M |
| 532 | M |
| 533 | M |
| 534 | M |
| 535 | C |
| 539 | B |
| 543 | B |
| 544 | C* |
| 545 | B |
| 546 | B |
| 547 | B |
| 551 | B |
| 552 | K |

TABLE 5B

| Cpd # | Synthetic procedure |
|---|---|
| 100 | A |
| 136 | 2 |
| 142 | A |
| 143 | A |
| 144 | A |
| 145 | A |
| 169 | A |
| 182 | K2 |
| 183 | K2 |
| 191 | K2 |
| 201 | A |
| 202 | A |
| 203 | A |
| 228 | A |
| 229 | A |
| 230 | A |
| 232 | A |
| 233 | A |
| 249 | A |
| 250 | A |
| 251 | A |
| 252 | A |
| 253 | A |
| 254 | A |
| 255 | A |
| 256 | A |
| 257 | A |
| 258 | A |
| 260 | A |
| 261 | A |
| 262 | B |
| 263 | A |
| 264 | A |
| 265 | A |
| 266 | A |
| 267 | A |
| 268 | A |
| 269 | A |
| 273 | A |
| 279 | A |
| 289 | A |
| 290 | A |
| 292 | A |
| 295 | K2 |
| 301 | A |
| 302 | A |
| 305 | B |
| 309 | A |
| 322 | M* |
| 323 | M* |
| 324 | M* |
| 325 | M* |
| 326 | B |
| 328 | B |
| 330 | L* |
| 336 | A |
| 337 | A |
| 338 | A |
| 342 | N |
| 343 | M* |
| 344 | N |
| 382 | N2 |
| 383 | N2 |
| 384 | N2 |
| 385 | N2 |
| 407 | N2 |
| 408 | N2 |
| 409 | N2 |
| 410 | N2 |
| 419 | A |
| 420 | A |
| 421 | A |
| 422 | A |
| 423 | A |
| 424 | A |
| 425 | A |
| 444 | C |
| 445 | C |
| 446 | C |
| 447 | C |
| 448 | C |
| 449 | C |
| 453 | N2 |
| 454 | N2 |
| 455 | N2 |
| 458 | N |
| 459 | N |
| 460 | N |
| 474 | N |
| 475 | N |
| 476 | N |
| 483 | C |
| 484 | C |
| 485 | C |
| 486 | C |
| 487 | C |
| 488 | C |
| 489 | C |
| 490 | C |
| 494 | N2 |
| 495 | N2 |
| 496 | N2 |
| 510 | N2 |
| 511 | N2 |
| 512 | N2 |
| 513 | N2 |
| 514 | N2 |
| 518 | S* |
| 519 | S* |
| 520 | N2 |
| 521 | N2 |
| 522 | N2 |
| 523 | M* |
| 526 | C |
| 536 | C |
| 537 | C |
| 538 | C |
| 540 | C |
| 541 | C |
| 542 | C |
| 548 | B |
| 549 | C |
| 550 | C |

### LCMS Method and Parameters:

### Instrument:

ACQUITY Premier QSM
ACQUITY Premier SM-FTN
ACQUITY Premier CM-A.
ACQUITY Premier PDA ex or ACQUITY Premier TUV
SQ Detector 2

### Mobile Phase A: 0.04% TFA in 4:1:45 MeCN/MeOH/H₂O

### Mobile Phase B: 0.04% TFA in 4:1:1.25 MeCN/MeOH/H₂O

Flow rate: 350 μL/min
Gradient program:

| t/min | % B |
|---|---|
| 0. | 5 |
| 2. | 5 |
| 4. | x * |
| 24.0 | x + 15 * |
| 24.1 | 100 |
| 26.0 | 100 |
| 26.1 | 5 |
| 30.0 | 5 |

* 5 ≤ x ≤ 60

Autosampler thermostat: 4° C.
Injection: 1 μg peptide of interest
Column: ACQUITY Premier Peptide CSH C18, 2.1 mm ø×150 mm (1.7 μm/130 Å)
Column thermostat: 60° C.

### Detection:

UV absorbance at 220 nm (12 Hz, unfiltered)
Mass analysis following electrospray ionization in positive mode (1.5 kV capillary; 40 V cone; desolvation at 350° C. with 750 L/h nitrogen) over a domain capturing the expected [M+2H]²⁺, [M+3H]³⁺ and similar species with a scan duration of 1 ms per m/z unit

Tables 6A and 6B below shows LCMS analysis for the compounds described herein.

TABLE 6A

| Cpd# | Molecular Weight | exact mass | observed mass | comment |
|---|---|---|---|---|
| 1 | 1716.02 | 1714.70 | 1715.80 | [M + H] |
| 2 | 2230.6 | 2228.97 | 744.88 | [M + 3H]/3 |
| 3 | 2469.87 | 2468.10 | 824.81 | [M + 3H]/3 |
| 4 | 2049.39 | 2047.95 | 738.35 | [M + 3H]/3 |
| 5 | 2437.85 | 2436.16 | 822.91 | [M + 3H]/3 |
| 6 | 1717.01 | 1715.68 | 1716.50 | [M + H] |
| 7 | 1697.99 | 1696.74 | 1697.70 | [M + H] |
| 8 | 1623.93 | 1622.67 | 812.60 | [M + 2H]/2 |
| 9 | 1730.05 | 1728.71 | 865.70 | [M + 2H]/2 |
| 10 | 1623.93 | 1622.67 | 1623.60 | [M + H] |
| 11 | 1623.93 | 1622.67 | 1623.60 | [M + H] |
| 12 | 1672.01 | 1670.71 | 1671.60 | [M + H] |
| 13 | 1672.01 | 1670.71 | 1671.50 | [M + H] |
| 14 | 1687.97 | 1686.67 | 1687.50 | [M + H] |
| 15 | 1687.97 | 1686.67 | 1687.40 | [M + H] |
| 16 | 1623.93 | 1622.67 | 812.70 | [M + 2H]/2 |
| 17 | 1600.89 | 1599.66 | 801.20 | [M + 2H]/2 |
| 18 | 1712.02 | 1710.76 | 1711.70 | [M + H] |
| 19 | 1697.99 | 1696.74 | 1697.70 | [M + H] |
| 20 | 1715.98 | 1714.73 | 858.50 | [M + 2H]/2 |
| 21 | 1732.43 | 1730.70 | 1731.40 | [M + H] |
| 22 | 1726 | 1724.74 | 1725.70 | [M + H] |
| 23 | 1682.98 | 1681.74 | 1682.80 | [M + H] |
| 24 | 1726 | 1724.74 | 1725.60 | [M + H] |
| 25 | 1682.98 | 1681.74 | 1682.70 | [M + H] |
| 26 | 1712.02 | 1710.76 | 856.50 | [M + 2H]/2 |
| 27 | 2313.67 | 2312.06 | 772.10 | [M + 3H]/3 |
| 28 | 2314.66 | 2313.05 | 2313.80 | [M + H] |
| 29 | 2342.67 | 2341.04 | 781.20 | [M + 3H]/3 |
| 30 | 2470.8 | 2469.10 | 824.50 | [M + 3H]/3 |
| 31 | 2341.68 | 2340.06 | 781.50 | [M + 3H]/3 |
| 32 | 1799.09 | 1797.79 | 1798.70 | [M + H] |
| 33 | 1799.09 | 1798.77 | 1799.80 | [M + H] |
| 34 | 1827.1 | 1825.78 | 1826.60 | [M + H] |
| 35 | 1827.1 | 1825.78 | 1826.60 | [M + H] |
| 36 | 1956.22 | 1954.83 | 1955.60 | [M + H] |
| 37 | 1655.91 | 1654.69 | 1655.60 | [M + H] |
| 38 | 1655.91 | 1654.69 | 1655.60 | [M + H] |
| 39 | 1687.97 | 1686.67 | 844.60 | [M + 2H]/2 |
| 40 | 1730.05 | 1728.71 | 865.70 | [M + 2H]/2 |
| 41 | 1730.05 | 1728.71 | 865.50 | [M + 2H]/2 |
| 42 | 1658.93 | 1657.64 | 1658.50 | [M + H] |
| 43 | 1658.93 | 1657.64 | 1658.70 | [M + H] |
| 44 | 1623.93 | 1622.67 | 1623.60 | [M + H] |
| 45 | 1600.89 | 1599.66 | 1600.10 | [M + H] |
| 46 | 1639.92 | 1638.67 | 1639.40 | [M + H] |
| 47 | 1639.92 | 1638.67 | 820.60 | [M + 2H]/2 |
| 48 | 1716.02 | 1714.70 | 858.60 | [M + 2H]/2 |
| 49 | 1716.02 | 1714.70 | 858.60 | [M + 2H]/2 |
| 50 | 2212.57 | 2211.02 | 2211.60 | [M + H] |
| 51 | 1729 | 1727.74 | 1728.50 | [M + H] |
| 52 | 1726.04 | 1724.77 | 1725.80 | [M + H] |
| 53 | 1770.05 | 1768.76 | 1769.70 | [M + H] |
| 54 | 2353.74 | 2352.10 | 785.40 | [M + 3H]/3 |
| 55 | 2353.74 | 2352.10 | 785.40 | [M + 3H]/3 |
| 56 | 2313.67 | 2312.06 | 772.30 | [M + 3H]/3 |
| 57 | 2298.66 | 2297.06 | 767.30 | [M + 3H]/3 |
| 58 | 2298.66 | 2297.06 | 767.00 | [M + 3H]/3 |
| 59 | 1817.09 | 1815.78 | 909.50 | [M + 2H]/2 |
| 60 | 1800.08 | 1798.78 | 901.10 | [M + 2H]/2 |
| 61 | 2327.7 | 2326.08 | 776.70 | [M + 3H]/3 |
| 62 | 2327.7 | 2326.08 | 776.70 | [M + 3H]/3 |
| 63 | 2299.65 | 2298.05 | 767.40 | [M + 3H]/3 |
| 64 | 2341.73 | 2340.10 | 2341.00 | [M + H] |
| 65 | 2355.71 | 2354.08 | 2355.00 | [M + H] |
| 66 | 2298.66 | 2297.05 | 1149.90 | [M + 2H]/2 |
| 67 | 2327.7 | 2326.08 | 776.96 | [M + 3H]/3 |
| 68 | 2327.7 | 2326.08 | 776.96 | [M + 3H]/3 |
| 69 | 2338.69 | 2337.06 | 780.63 | [M + 3H]/3 |
| 70 | 2338.69 | 2337.06 | 780.56 | [M + 3H]/3 |
| 71 | 2299.65 | 2298.05 | 767.58 | [M + 3H]/3 |
| 72 | 2341.73 | 2340.10 | 781.61 | [M + 3H]/3 |
| 73 | 2345.69 | 2344.07 | 782.96 | [M + 3H]/3 |
| 74 | 2341.73 | 2340.10 | 781.61 | [M + 3H]/3 |
| 75 | 2355.76 | 2354.11 | 786.26 | [M + 3H]/3 |
| 76 | 2359.72 | 2358.09 | 787.61 | [M + 3H]/3 |
| 77 | 2359.72 | 2358.09 | 787.61 | [M + 3H]/3 |
| 78 | 2373.75 | 2372.10 | 792.26 | [M + 3H]/3 |
| 79 | 2333.66 | 2332.03 | 778.80 | [M + 3H]/3 |
| 80 | 2312.69 | 2311.08 | 2311.90 | [M + H] |
| 81 | 2285.66 | 2284.07 | 2285.70 | [M + H] |
| 82 | 2319.72 | 2318.11 | 773.90 | [M + 3H]/3 |
| 83 | 2342.72 | 2341.09 | 781.60 | [M + 3H]/3 |
| 84 | 2342.72 | 2341.09 | 781.60 | [M + 3H]/3 |
| 85 | 2341.69 | 2340.07 | 781.40 | [M + 3H]/3 |
| 86 | 2340.75 | 2339.11 | 781.00 | [M + 3H]/3 |
| 87 | 2326.72 | 2325.10 | 776.30 | [M + 3H]/3 |
| 88 | 2298.66 | 2297.06 | 767.00 | [M + 3H]/3 |
| 89 | 2366.78 | 2365.13 | 789.80 | [M + 3H]/3 |
| 90 | 2340.75 | 2339.11 | 780.90 | [M + 3H]/3 |
| 91 | 2341.68 | 2340.06 | 781.30 | [M + 3H]/3 |
| 92 | 2299.65 | 2298.05 | 767.30 | [M + 3H]/3 |
| 93 | 2327.66 | 2326.04 | 776.60 | [M + 3H]/3 |
| 94 | 2566.97 | 2565.21 | 856.30 | [M + 3H]/3 |
| 95 | 2327.7 | 2326.08 | 776.88 | [M + 3H]/3 |
| 96 | 2299.65 | 2298.05 | 767.51 | [M + 3H]/3 |
| 97 | 2327.7 | 2326.08 | 776.88 | [M + 3H]/3 |
| 98 | 2180.53 | 2179.01 | 728.50 | [M + 3H]/3 |
| 99 | 2141.49 | 2140.00 | 715.53 | [M + 3H]/3 |
| 101 | 2320.71 | 2319.12 | 581.10 | [M + 4H]/4 |
| 102 | 2342.71 | 2341.08 | 781.80 | [M + 3H]/3 |
| 103 | 2329.63 | 2328.01 | 1165.90 | [M + 2H]/2 |
| 104 | 2400.75 | 2399.09 | 801.00 | [M + 3H]/3 |
| 105 | 2358.67 | 2357.04 | 787.00 | [M + 3H]/3 |
| 106 | 2294.62 | 2293.04 | 765.70 | [M + 3H]/3 |
| 107 | 2344.64 | 2343.02 | 782.40 | [M + 3H]/3 |
| 108 | 2336.71 | 2335.11 | 585.10 | [M + 4H]/4 |
| 109 | 2297.68 | 2296.10 | 575.30 | [M + 4H]/4 |
| 110 | 2320.71 | 2319.12 | 581.10 | [M + 4H]/4 |
| 111 | 2370.73 | 2369.10 | 593.50 | [M + 4H]/4 |
| 112 | 2310.62 | 2309.04 | 771.00 | [M + 3H]/3 |
| 113 | 2271.59 | 2270.03 | 758.00 | [M + 3H]/3 |
| 114 | 2294.62 | 2293.04 | 765.70 | [M + 3H]/3 |
| 115 | 2358.67 | 2357.04 | 786.90 | [M + 3H]/3 |
| 116 | 2294.62 | 2293.04 | 765.70 | [M + 3H]/3 |
| 117 | 2355.76 | 2354.11 | 786.33 | [M + 3H]/3 |
| 118 | 2355.76 | 2354.11 | 786.26 | [M + 3H]/3 |
| 119 | 2325.69 | 2324.06 | 776.28 | [M + 3H]/3 |
| 120 | 2329.67 | 2328.06 | 777.63 | [M + 3H]/3 |
| 121 | 2341.73 | 2340.10 | 781.61 | [M + 3H]/3 |
| 122 | 2343.7 | 2342.08 | 782.28 | [M + 3H]/3 |
| 123 | 2325.69 | 2324.06 | 776.21 | [M + 3H]/3 |
| 124 | 2313.67 | 2312.06 | 772.31 | [M + 3H]/3 |
| 125 | 2329.67 | 2328.06 | 777.63 | [M + 3H]/3 |
| 126 | 2341.73 | 2340.10 | 781.61 | [M + 3H]/3 |
| 127 | 2343.7 | 2342.08 | 782.21 | [M + 3H]/3 |
| 128 | 2324.7 | 2323.07 | 2324.70 | [M + H] |
| 129 | 2324.7 | 2323.07 | 2324.50 | [M + H] |
| 130 | 2327.7 | 2326.08 | 776.80 | [M + 3H]/3 |
| 131 | 2312.69 | 2311.08 | 2312.60 | [M + H] |
| 132 | 2327.7 | 2326.08 | 2327.00 | [M + H] |
| 133 | 2331.67 | 2330.06 | 2331.10 | [M + H] |
| 134 | 2314.66 | 2313.06 | 2314.00 | [M + H] |
| 135 | 2318.74 | 2317.13 | 773.80 | [M + 3H]/3 |
| 137 | 2313.67 | 2312.06 | 1157.50 | [M + 2H]/2 |
| 138 | 2367.77 | 2366.11 | 790.38 | [M + 3H]/3 |
| 139 | 2369.74 | 2368.09 | 791.08 | [M + 3H]/3 |
| 140 | 2383.77 | 2382.11 | 795.70 | [M + 3H]/3 |
| 141 | 2323.71 | 2322.09 | 775.67 | [M + 3H]/3 |
| 146 | 2359.72 | 2358.09 | 787.44 | [M + 3H]/3 |
| 147 | 2345.69 | 2344.07 | 783.02 | [M + 3H]/3 |
| 148 | 2345.69 | 2344.07 | 782.95 | [M + 3H]/3 |
| 149 | 2341.73 | 2340.10 | 781.80 | [M + 3H]/3 |
| 150 | 2341.73 | 2340.10 | 781.80 | [M + 3H]/3 |
| 151 | 2345.69 | 2344.07 | 783.10 | [M + 3H]/3 |
| 152 | 2366.78 | 2365.13 | 2366.30 | [M + H] |
| 153 | 2352.76 | 2351.11 | 2352.20 | [M + H] |
| 154 | 2353.74 | 2352.10 | 2353.30 | [M + H] |
| 155 | 2312.69 | 2311.08 | 2312.30 | [M + H] |
| 156 | 2328.69 | 2327.08 | 777.60 | [M + 3H]/3 |
| 157 | 2326.72 | 2325.10 | 776.80 | [M + 3H]/3 |
| 158 | 2338.72 | 2337.09 | 2338.90 | [M + H] |
| 159 | 2338.72 | 2337.09 | 2338.80 | [M + H] |
| 160 | 2341.7 | 2340.08 | 781.62 | [M + 3H]/3 |
| 161 | 2357.7 | 2356.07 | 786.69 | [M + 3H]/3 |
| 162 | 2345.69 | 2344.07 | 783.02 | [M + 3H]/3 |
| 163 | 2345.69 | 2344.07 | 783.02 | [M + 3H]/3 |
| 164 | 2363.68 | 2362.06 | 1182.88 | [M + 2H]/2 |
| 165 | 2359.72 | 2358.09 | 787.72 | [M + 3H]/3 |
| 166 | 2389.7 | 2388.06 | 797.66 | [M + 3H]/3 |
| 167 | 2359.72 | 2358.09 | 787.65 | [M + 3H]/3 |
| 168 | 2359.72 | 2358.09 | 787.72 | [M + 3H]/3 |
| 170 | 2330.68 | 2329.07 | 777.70 | [M + 3H]/3 |
| 171 | 2359.72 | 2358.09 | 787.79 | [M + 3H]/3 |
| 172 | 2347.68 | 2346.07 | 783.72 | [M + 3H]/3 |
| 173 | 2363.68 | 2362.06 | 789.05 | [M + 3H]/3 |
| 174 | 2373.75 | 2372.10 | 792.41 | [M + 3H]/3 |
| 175 | 2325.7 | 2324.10 | 776.37 | [M + 3H]/3 |
| 176 | 2325.7 | 2324.10 | 776.23 | [M + 3H]/3 |
| 177 | 2355.76 | 2354.11 | 786.46 | [M + 3H]/3 |
| 178 | 2283.62 | 2282.06 | 762.37 | [M + 3H]/3 |
| 179 | 2352.75 | 2351.10 | 785.27 | [M + 3H]/3 |
| 180 | 2392.82 | 2391.13 | 798.71 | [M + 3H]/3 |
| 181 | 2399.78 | 2398.12 | 801.02 | [M + 3H]/3 |
| 184 | 2345.69 | 2344.07 | 783.10 | [M + 3H]/3 |
| 185 | 2345.69 | 2344.07 | 2345.80 | [M + H] |
| 186 | 2359.72 | 2358.09 | 787.90 | [M + 3H]/3 |
| 187 | 2359.72 | 2358.09 | 2359.50 | [M + H] |
| 188 | 2359.72 | 2358.09 | 2359.50 | [M + H] |
| 189 | 2359.72 | 2358.09 | 787.90 | [M + 3H]/3 |
| 190 | 2370.75 | 2369.10 | 2370.20 | [M + H] |
| 192 | 2347.68 | 2346.07 | 784.20 | [M + 3H]/3 |
| 193 | 2359.72 | 2358.09 | 788.19 | [M + 3H]/3 |
| 194 | 2359.72 | 2358.09 | 787.98 | [M + 3H]/3 |
| 195 | 2359.72 | 2358.09 | 787.91 | [M + 3H]/3 |
| 196 | 2309.7 | 2308.11 | 771.25 | [M + 3H]/3 |
| 197 | 2309.7 | 2308.11 | 771.25 | [M + 3H]/3 |
| 198 | 2283.62 | 2282.06 | 762.57 | [M + 3H]/3 |
| 199 | 2283.62 | 2282.06 | 762.43 | [M + 3H]/3 |
| 200 | 2337.71 | 2336.10 | 780.56 | [M + 3H]/3 |
| 204 | 2309.66 | 2308.07 | 771.25 | [M + 3H]/3 |
| 205 | 2359.72 | 2358.09 | 788.19 | [M + 3H]/3 |
| 206 | 2335.74 | 2334.12 | 779.93 | [M + 3H]/3 |
| 207 | 2363.75 | 2362.12 | 789.24 | [M + 3H]/3 |
| 208 | 2335.74 | 2334.12 | 779.86 | [M + 3H]/3 |
| 209 | 2321.71 | 2320.11 | 775.24 | [M + 3H]/3 |
| 210 | 2309.66 | 2308.07 | 771.18 | [M + 3H]/3 |
| 211 | 2323.69 | 2322.09 | 775.87 | [M + 3H]/3 |
| 212 | 2347.75 | 2346.12 | 783.92 | [M + 3H]/3 |
| 213 | 2349.72 | 2348.10 | 784.48 | [M + 3H]/3 |
| 214 | 2260.59 | 2259.02 | 2260.40 | [M + H] |
| 215 | 2385.76 | 2384.10 | 2385.20 | [M + H] |
| 216 | 2381.79 | 2380.13 | 2381.00 | [M + H] |
| 217 | 2359.72 | 2358.09 | 788.10 | [M + 3H]/3 |
| 218 | 2230.56 | 2229.01 | 2230.30 | [M + H] |
| 219 | 2337.74 | 2336.10 | 780.42 | [M + 3H]/3 |
| 220 | 2327.79 | 2326.15 | 777.20 | [M + 3H]/3 |
| 221 | 2284.68 | 2283.09 | 762.99 | [M + 3H]/3 |
| 222 | 2275.67 | 2274.09 | 759.98 | [M + 3H]/3 |
| 223 | 2329.76 | 2328.13 | 777.90 | [M + 3H]/3 |
| 224 | 2301.75 | 2300.14 | 768.59 | [M + 3H]/3 |
| 225 | 2287.73 | 2286.12 | 764.04 | [M + 3H]/3 |
| 226 | 2275.67 | 2274.09 | 759.84 | [M + 3H]/3 |
| 227 | 2375.81 | 2374.15 | 793.24 | [M + 3H]/3 |
| 231 | 2289.7 | 2288.10 | 764.60 | [M + 3H]/3 |
| 234 | 2318.67 | 2317.07 | 774.33 | [M + 3H]/3 |
| 235 | 2376.17 | 2374.06 | 793.29 | [M + 3H]/3 |
| 236 | 2361.78 | 2360.14 | 788.54 | [M + 3H]/3 |
| 237 | 2359.72 | 2358.09 | 787.90 | [M + 3H]/3 |
| 238 | 2293.66 | 2292.08 | 2294.00 | [M + H] |
| 239 | 2385.76 | 2384.10 | 2386.60 | [M + H] |
| 240 | 2330.68 | 2329.07 | 2331.30 | [M + H] |
| 241 | 2348.62 | 2347.14 | 2348.20 | [M + H] |
| 242 | 2348.62 | 2347.14 | 2348.40 | [M + H] |
| 243 | 2313.76 | 2312.14 | 772.58 | [M + 3H]/3 |
| 244 | 2348.76 | 2347.11 | 784.27 | [M + 3H]/3 |
| 245 | 2334.74 | 2333.09 | 779.65 | [M + 3H]/3 |
| 246 | 2362.79 | 2361.12 | 788.89 | [M + 3H]/3 |
| 247 | 2348.76 | 2347.11 | 784.41 | [M + 3H]/3 |
| 248 | 2355.73 | 2354.09 | 786.72 | [M + 3H]/3 |
| 259 | 2574.05 | 2572.26 | 859.52 | [M + 3H]/3 |
| 271 | 2333.73 | 2332.11 | 779.42 | [M + 3H]/3 |
| 272 | 2585.95 | 2584.17 | 863.52 | [M + 3H]/3 |
| 274 | 2469.9 | 2468.18 | 824.58 | [M + 3H]/3 |
| 275 | 2599.02 | 2597.22 | 867.79 | [M + 3H]/3 |
| 276 | 2728.13 | 2726.26 | 910.92 | [M + 3H]/3 |
| 277 | 2493.97 | 2492.22 | 832.71 | [M + 3H]/3 |
| 278 | 2752.2 | 2750.30 | 918.70 | [M + 3H]/3 |
| 280 | 2750.26 | 2748.37 | 918.28 | [M + 3H]/3 |
| 28 | 2774.33 | 2772.40 | 926.40 | [M + 3H]/3 |
| 282 | 2566.99 | 2565.23 | 857.07 | [M + 3H]/3 |
| 283 | 2431.87 | 2430.17 | 812.26 | [M + 3H]/3 |
| 284 | 2349.75 | 2348.10 | 784.74 | [M + 3H]/3 |
| 285 | 2649.04 | 2647.23 | 884.31 | [M + 3H]/3 |
| 286 | 2395.75 | 2394.09 | 799.93 | [M + 3H]/3 |
| 287 | 2319.7 | 2318.09 | 774.80 | [M + 3H]/3 |
| 288 | 2334.71 | 2333.09 | 779.49 | [M + 3H]/3 |
| 291 | 2369.71 | 2368.07 | 791.18 | [M + 3H]/3 |
| 293 | 2572.99 | 2571.23 | 859.03 | [M + 3H]/3 |
| 294 | 2623.01 | 2621.21 | 875.70 | [M + 3H]/3 |
| 297 | 2351.77 | 2350.12 | 2351.70 | [M + H] |
| 298 | 2259.67 | 2258.09 | 2259.60 | [M + H] |
| 299 | 2274.68 | 2273.09 | 2274.20 | [M + H] |
| 300 | 2307.76 | 2306.13 | 2307.70 | [M + H] |
| 303 | 2366.75 | 2365.12 | 790.62 | [M + 3H]/3 |
| 304 | 2348.74 | 2347.11 | 784.39 | [M + 3H]/3 |
| 306 | 2411.79 | 2410.13 | 805.40 | [M + 3H]/3 |
| 307 | 2582 | 2580.23 | 862.26 | [M + 3H]/3 |
| 308 | 2600.02 | 2598.24 | 867.93 | [M + 3H]/3 |
| 310 | 2600.02 | 2598.24 | 868.14 | [M + 3H]/3 |
| 311 | 2321.67 | 2320.06 | 1162.15 | [M + 2H]/2 |
| 312 | 2334.71 | 2333.09 | 779.59 | [M + 3H]/3 |
| 313 | 2396.74 | 2395.07 | 800.25 | [M + 3H]/3 |
| 314 | 2409.78 | 2408.10 | 804.59 | [M + 3H]/3 |
| 315 | 2402.81 | 2401.12 | 802.42 | [M + 3H]/3 |
| 318 | 2450.83 | 2449.14 | 818.24 | [M + 3H]/3 |
| 319 | 2432.81 | 2431.13 | 812.29 | [M + 3H]/3 |
| 320 | 2453.79 | 2452.09 | 819.57 | [M + 3H]/3 |
| 321 | 2398.79 | 2397.14 | 800.88 | [M + 3H]/3 |
| 327 | 2332.73 | 2331.13 | 778.82 | [M + 3H]/3 |
| 329 | 2335.66 | 2334.17 | 2335.70 | [M + H] |
| 331 | 2338.7 | 2337.09 | 2338.80 | [M + H] |
| 332 | 2352.72 | 2351.10 | 2352.80 | [M + H] |
| 333 | 2318.71 | 2317.12 | 2318.90 | [M + H] |
| 334 | 2318.71 | 2317.12 | 2318.80 | [M + H] |
| 335 | 2318.71 | 2317.12 | 2318.80 | [M + H] |
| 339 | 2450.83 | 2449.14 | 818.38 | [M + 3H]/3 |
| 340 | 2392.79 | 2391.13 | 798.92 | [M + 3H]/3 |
| 34 | 3377.91 | 3375.67 | 1126.83 | [M + 3H]/3 |
| 345 | 2324.71 | 2323.11 | 2324.60 | [M + H] |
| 347 | 2366.75 | 2365.12 | 2366.40 | [M + H] |
| 348 | 2380.78 | 2379.13 | 2380.40 | [M + H] |
| 349 | 2335.69 | 2334.08 | 2335.40 | [M + H] |
| 350 | 2349.72 | 2348.09 | 2349.30 | [M + H] |
| 351 | 2366.75 | 2365.12 | 2366.80 | [M + H] |
| 352 | 2366.75 | 2365.12 | 2366.60 | [M + H] |
| 353 | 2366.75 | 2365.12 | 2366.80 | [M + H] |
| 354 | 2366.75 | 2365.12 | 2366.60 | [M + H] |
| 355 | 2366.75 | 2365.12 | 2367.00 | [M + H] |
| 356 | 2366.75 | 2365.12 | 2366.90 | [M + H] |
| 357 | 2316.74 | 2315.14 | 2316.70 | [M + H] |
| 358 | 2366.75 | 2365.12 | 2366.90 | [M + H] |
| 359 | 2309.72 | 2308.12 | 2309.60 | [M + H] |
| 360 | 2332.73 | 2331.13 | 2332.90 | [M + H] |
| 361 | 2352.72 | 2351.10 | 2352.80 | [M + H] |
| 362 | 2352.72 | 2351.10 | 2352.90 | [M + H] |
| 363 | 3050.55 | 3048.48 | 1018.19 | [M + 3H]/3 |
| 364 | 2898.14 | 2896.23 | 967.65 | [M + 3H]/3 |
| 365 | 3020.3 | 3018.32 | 1008.35 | [M + 3H]/3 |
| 366 | 2365.77 | 2364.13 | 790.22 | [M + 3H]/3 |
| 367 | 2392.79 | 2391.13 | 798.89 | [M + 3H]/3 |
| 368 | 2406.82 | 2405.15 | 803.64 | [M + 3H]/3 |
| 369 | 2366.75 | 2365.12 | 790.36 | [M + 3H]/3 |
| 370 | 2354.7 | 2353.08 | 786.16 | [M + 3H]/3 |
| 371 | 2338.7 | 2337.09 | 780.30 | [M + 3H]/3 |
| 372 | 2324.67 | 2323.07 | 775.70 | [M + 3H]/3 |
| 373 | 2368.72 | 2367.10 | 790.30 | [M + 3H]/3 |
| 374 | 2277.61 | 2276.03 | 1139.40 | [M + 2H]/2 |
| 375 | 2321.67 | 2320.06 | 775.30 | [M + 3H]/3 |
| 376 | 2335.69 | 2334.08 | 2335.60 | [M + H] |
| 377 | 2368.72 | 2367.10 | 790.90 | [M + 3H]/3 |
| 378 | 2354.7 | 2353.08 | 786.20 | [M + 3H]/3 |
| 379 | 2368.72 | 2367.10 | 2368.80 | [M + H] |
| 380 | 2384.74 | 2383.11 | 2384.80 | [M + H] |
| 381 | 2384.74 | 2383.11 | 2384.80 | [M + H] |
| 386 | 2368.72 | 2367.10 | 790.30 | [M + 3H]/3 |
| 389 | 2448.8 | 2447.07 | 817.10 | [M + 3H]/3 |
| 390 | 2496.89 | 2495.11 | 833.00 | [M + 3H]/3 |
| 391 | 2619.06 | 2617.27 | 873.80 | [M + 3H]/3 |
| 392 | 2425.78 | 2424.12 | 809.30 | [M + 3H]/3 |
| 393 | 2425.78 | 2424.12 | 809.30 | [M + 3H]/3 |
| 394 | 2439.8 | 2438.13 | 814.00 | [M + 3H]/3 |
| 395 | 2439.8 | 2438.13 | 814.00 | [M + 3H]/3 |
| 396 | 2406.8 | 2405.13 | 803.93 | [M + 3H]/3 |
| 397 | 2380.78 | 2379.13 | 794.92 | [M + 3H]/3 |
| 398 | 2378.76 | 2377.12 | 794.28 | [M + 3H]/3 |
| 399 | 2366.75 | 2365.12 | 1184.40 | [M + 2H]/2 |
| 400 | 2393.78 | 2392.13 | 1197.90 | [M + 2H]/2 |
| 401 | 2356.72 | 2355.11 | 786.60 | [M + 3H]/3 |
| 402 | 2367.74 | 2366.11 | 790.40 | [M + 3H]/3 |
| 403 | 2367.74 | 2366.11 | 790.50 | [M + 3H]/3 |
| 404 | 1851.19 | 1849.86 | 1851.20 | [M + H] |
| 405 | 2764.26 | 2762.36 | 923.07 | [M + 3H]/3 |
| 427 | 2434.83 | 2433.15 | 812.40 | [M + 3H]/3 |
| 428 | 2521.9 | 2520.18 | 841.40 | [M + 3H]/3 |
| 429 | 2521.9 | 2520.18 | 841.40 | [M + 3H]/3 |
| 430 | 2427.23 | 2425.09 | 809.80 | [M + 3H]/3 |
| 431 | 2408.79 | 2407.13 | 803.70 | [M + 3H]/3 |
| 432 | 2406.82 | 2405.15 | 803.00 | [M + 3H]/3 |
| 433 | 2406.82 | 2405.15 | 803.00 | [M + 3H]/3 |
| 434 | 2406.82 | 2405.15 | 803.00 | [M + 3H]/3 |
| 435 | 2331.73 | 2330.10 | 778.10 | [M + 3H]/3 |
| 436 | 2385.82 | 2384.15 | 796.10 | [M + 3H]/3 |
| 437 | 2428.84 | 2427.10 | 810.30 | [M + 3H]/3 |
| 438 | 2464.85 | 2463.15 | 822.50 | [M + 3H]/3 |
| 439 | 2464.85 | 2463.15 | 822.30 | [M + 3H]/3 |
| 440 | 2521.9 | 2520.18 | 841.84 | [M + 3H]/3 |
| 441 | 2514.94 | 2513.19 | 839.46 | [M + 3H]/3 |
| 442 | 2432.85 | 2431.16 | 812.14 | [M + 3H]/3 |
| 443 | 2404.8 | 2403.13 | 803.17 | [M + 3H]/3 |
| 450 | 2406.82 | 2405.15 | 803.50 | [M + 3H]/3 |
| 451 | 2450.83 | 2449.14 | 818.09 | [M + 3H]/3 |
| 452 | 2520.92 | 2519.19 | 841.55 | [M + 3H]/3 |
| 456 | 2406.82 | 2405.15 | 803.10 | [M + 3H]/3 |
| 457 | 2409.24 | 2407.10 | 803.80 | [M + 3H]/3 |
| 461 | 2392.79 | 2391.13 | 798.30 | [M + 3H]/3 |
| 462 | 2388.83 | 2387.16 | 797.00 | [M + 3H]/3 |
| 463 | 2404.82 | 2403.15 | 802.40 | [M + 3H]/3 |
| 464 | 2535.93 | 2534.19 | 846.45 | [M + 3H]/3 |
| 465 | 2640.08 | 2638.28 | 881.13 | [M + 3H]/3 |
| 467 | 2392.79 | 2391.13 | 798.50 | [M + 3H]/3 |
| 468 | 2393.78 | 2392.13 | 798.90 | [M + 3H]/3 |
| 469 | 2408.79 | 2407.13 | 803.70 | [M + 3H]/3 |
| 470 | 2410.78 | 2409.12 | 804.50 | [M + 3H]/3 |
| 471 | 2388.83 | 2387.16 | 797.00 | [M + 3H]/3 |
| 472 | 2409.24 | 2407.10 | 803.80 | [M + 3H]/3 |
| 473 | 2388.83 | 2387.16 | 797.10 | [M + 3H]/3 |
| 477 | 2461.85 | 2460.15 | 821.40 | [M + 3H]/3 |
| 478 | 2474.84 | 2473.09 | 825.70 | [M + 3H]/3 |
| 479 | 2406.82 | 2405.15 | 803.44 | [M + 3H]/3 |
| 480 | 2406.82 | 2405.15 | 803.44 | [M + 3H]/3 |
| 481 | 2661.1 | 2659.28 | 888.11 | [M + 3H]/3 |
| 482 | 2756.27 | 2754.29 | 919.85 | [M + 3H]/3 |
| 491 | 2406.82 | 2405.15 | 803.00 | [M + 3H]/3 |
| 492 | 2388.83 | 2387.16 | 797.00 | [M + 3H]/3 |
| 493 | 2404.82 | 2403.15 | 802.40 | [M + 3H]/3 |
| 497 | 2464.85 | 2463.15 | 822.71 | [M + 3H]/3 |
| 498 | 2535.93 | 2534.19 | 846.46 | [M + 3H]/3 |
| 499 | 2506.89 | 2505.18 | 836.66 | [M + 3H]/3 |
| 500 | 2696.15 | 2694.31 | 899.77 | [M + 3H]/3 |
| 501 | 2535.93 | 2534.19 | 846.39 | [M + 3H]/3 |
| 502 | 2405.83 | 2404.16 | 803.03 | [M + 3H]/3 |
| 503 | 2577.97 | 2576.21 | 860.40 | [M + 3H]/3 |
| 504 | 2517.92 | 2516.19 | 840.00 | [M + 3H]/3 |
| 505 | 2647.04 | 2645.23 | 883.30 | [M + 3H]/3 |
| 506 | 2448.86 | 2447.17 | 817.20 | [M + 3H]/3 |
| 507 | 2475.88 | 2474.17 | 826.10 | [M + 3H]/3 |
| 508 | 2577.97 | 2576.21 | 860.40 | [M + 3H]/3 |
| 509 | 2406.82 | 2405.15 | 803.00 | [M + 3H]/3 |
| 515 | 2695.16 | 2693.32 | 899.49 | [M + 3H]/3 |
| 516 | 2399.85 | 2398.16 | 801.07 | [M + 3H]/3 |
| 517 | 2770.29 | 2768.31 | 924.20 | [M + 3H]/3 |
| 524 | 2442.86 | 2441.12 | 815.34 | [M + 3H]/3 |
| 525 | 2607.99 | 2606.21 | 870.63 | [M + 3H]/3 |
| 527 | 2420.84 | 2419.16 | 807.70 | [M + 3H]/3 |
| 528 | 2549.96 | 2548.21 | 850.70 | [M + 3H]/3 |
| 529 | 2531.94 | 2530.20 | 844.70 | [M + 3H]/3 |
| 530 | 2693.1 | 2691.27 | 898.50 | [M + 3H]/3 |
| 531 | 2420.84 | 2419.16 | 807.70 | [M + 3H]/3 |
| 532 | 2463.87 | 2462.17 | 822.00 | [M + 3H]/3 |
| 533 | 2521.9 | 2520.18 | 841.40 | [M + 3H]/3 |
| 534 | 2505.91 | 2504.18 | 836.10 | [M + 3H]/3 |
| 535 | 2753.2 | 2751.32 | 919.11 | [M + 3H]/3 |
| 539 | 2388.8 | 2387.14 | 797.36 | [M + 3H]/3 |
| 543 | 2836.24 | 2834.32 | 946.49 | [M + 3H]/3 |
| 544 | 2650.03 | 2648.22 | 884.43 | [M + 3H]/3 |
| 545 | 2737.11 | 2735.26 | 913.68 | [M + 3H]/3 |
| 546 | 2747.19 | 2745.31 | 916.72 | [M + 3H]/3 |
| 547 | 2650.04 | 2648.23 | 884.36 | [M + 3H]/3 |
| 551 | 2636.01 | 2634.22 | 879.74 | [M + 3H]/3 |
| 552 | 2298.71 | 2297.10 | 766.90 | [M + 3H]/3 |

TABLE 6B

| | Molecular | exact | observed | |
|---|---|---|---|---|
| Cpd# | Weight | mass | mass | comment |
| 100 | 2270.65 | 2269.06 | 757.90 | [M + 3H]/3 |
| 136 | 2313.67 | 2312.06 | 772.30 | [M + 3H]/3 |
| 142 | 2164.53 | 2163.02 | 722.73 | [M + 3H]/3 |
| 143 | 2228.57 | 2227.01 | 744.02 | [M + 3H]/3 |
| 144 | 2164.53 | 2163.02 | 722.38 | [M + 3H]/3 |
| 145 | 2199.53 | 2197.99 | 1100.73 | [M + 2H]/2 |
| 169 | 2272.6 | 2271.02 | 758.59 | [M + 3H]/3 |
| 182 | 2230.56 | 2229.01 | 2230.70 | [M + H] |
| 183 | 2230.56 | 2229.01 | 744.60 | [M + 3H]/3 |
| 191 | 2260.59 | 2259.02 | 2260.10 | [M + H] |
| 201 | 2221.59 | 2220.04 | 1112.21 | [M + 2H]/2 |
| 202 | 2222.58 | 2221.04 | 742.19 | [M + 3H]/3 |
| 203 | 2236.61 | 2235.05 | 746.81 | [M + 3H]/3 |
| 228 | 2202.51 | 2200.98 | 1102.96 | [M + 2H]/2 |
| 229 | 2216.53 | 2214.99 | 740.16 | [M + 3H]/3 |
| 230 | 2230.56 | 2229.01 | 744.92 | [M + 3H]/3 |
| 232 | 2221.59 | 2220.04 | 1112.56 | [M + 2H]/2 |
| 233 | 2207.57 | 2206.03 | 1105.35 | [M + 2H]/2 |
| 249 | 2212.55 | 2211.00 | 738.98 | [M + 3H]/3 |
| 250 | 2221.58 | 2220.01 | 741.98 | [M + 3H]/3 |
| 251 | 2228.54 | 2226.99 | 744.23 | [M + 3H]/3 |
| 252 | 2334.69 | 2333.05 | 779.60 | [M + 3H]/3 |
| 253 | 2463.81 | 2462.10 | 822.78 | [M + 3H]/3 |
| 254 | 2362.75 | 2361.09 | 789.10 | [M + 3H]/3 |
| 255 | 2491.86 | 2490.13 | 832.01 | [M + 3H]/3 |
| 256 | 2438.84 | 2437.14 | 814.39 | [M + 3H]/3 |
| 257 | 2466.9 | 2465.17 | 823.62 | [M + 3H]/3 |
| 258 | 2620.98 | 2619.17 | 875.32 | [M + 3H]/3 |
| 260 | 2212.55 | 2211.00 | 738.96 | [M + 3H]/3 |
| 261 | 2219.58 | 2218.03 | 741.05 | [M + 3H]/3 |
| 262 | 2227.56 | 2226.00 | 743.91 | [M + 3H]/3 |
| 263 | 2224.55 | 2222.99 | 742.54 | [M + 3H]/3 |
| 264 | 2224.55 | 2222.99 | 742.74 | [M + 3H]/3 |
| 265 | 2220.59 | 2219.01 | 1111.49 | [M + 2H]/2 |
| 266 | 2227.56 | 2226.00 | 743.58 | [M + 3H]/3 |
| 267 | 2229.53 | 2227.98 | 744.23 | [M + 3H]/3 |
| 268 | 2231.52 | 2229.97 | 745.01 | [M + 3H]/3 |
| 269 | 2227.56 | 2226.00 | 743.98 | [M + 3H]/3 |
| 273 | 2219.58 | 2218.03 | 741.95 | [M + 3H]/3 |
| 279 | 2643.11 | 2641.27 | 882.42 | [M + 3H]/3 |
| 289 | 2241.54 | 2239.98 | 748.33 | [M + 3H]/3 |
| 290 | 2242.52 | 2240.96 | 749.10 | [M + 3H]/3 |
| 292 | 2205.55 | 2204.01 | 736.49 | [M + 3H]/3 |
| 295 | 2260.59 | 2259.02 | 2260.20 | [M + H] |
| 301 | 2282.64 | 2281.04 | 762.33 | [M + 3H]/3 |
| 302 | 2191.52 | 2190.00 | 731.80 | [M + 3H]/3 |
| 305 | 2220.56 | 2219.01 | 741.67 | [M + 3H]/3 |
| 309 | 2205.58 | 2204.01 | 736.62 | [M + 3H]/3 |
| 322 | 2238.58 | 2237.02 | 1120.00 | [M + 2H]/2 |
| 323 | 2237.59 | 2236.04 | 1117.50 | [M − 2H]/2, |
| | | | | negative mode |
| 324 | 2295.63 | 2294.04 | 1148.50 | [M + 2H]/2 |
| 325 | 2260.54 | 2258.97 | 1131.00 | [M + 2H]/2 |
| 326 | 2281.6 | 2280.03 | 762.08 | [M + 3H]/3 |
| 328 | 2224.55 | 2223.01 | 1113.90 | [M + 2H]/2 |
| 330 | 2219.6 | 2218.03 | 2219.60 | [M + H] |
| 336 | 2203.58 | 2202.05 | 1103.46 | [M + 2H]/2 |
| 337 | 2365.77 | 2364.13 | 790.02 | [M + 3H]/3 |
| 338 | 2237.59 | 2236.04 | 1120.55 | [M + 2H]/2 |
| 342 | 2264.61 | 2263.04 | 1133.00 | [M + 2H]/2 |
| 343 | 2295.63 | 2294.04 | 1148.50 | [M + 2H]/2 |
| 344 | 2237.59 | 2236.04 | 746.60 | [M + 3H]/3 |
| 382 | 2510.92 | 2509.20 | 1254.10 | [M − 2H]/2, |
| | | | | negative mode |
| 383 | 2483.9 | 2482.20 | 828.70 | [M + 3H]/3 |
| 384 | 2582 | 2580.23 | 1289.60 | [M − 2H]/2, |
| | | | | negative mode |
| 385 | 2554.98 | 2553.23 | 852.40 | [M + 3H]/3 |
| 407 | 2598 | 2596.23 | 1299.90 | [M + 2H]/2 |
| 408 | 2570.98 | 2569.23 | 857.70 | [M + 3H]/3 |
| 409 | 2640.04 | 2638.24 | 1320.90 | [M + 2H]/2 |
| 410 | 2613.01 | 2611.24 | 871.70 | [M + 3H]/3 |
| 419 | 2294.64 | 2293.06 | 766.27 | [M + 3H]/3 |
| 420 | 2381.72 | 2380.09 | 795.19 | [M + 3H]/3 |
| 421 | 2423.76 | 2422.10 | 809.19 | [M + 3H]/3 |
| 422 | 2324.67 | 2323.07 | 1164.02 | [M + 2H]/2 |
| 423 | 2366.71 | 2365.08 | 1185.10 | [M + 2H]/2 |
| 424 | 2380.73 | 2379.10 | 1192.03 | [M + 2H]/2 |
| 425 | 2380.73 | 2379.10 | 1191.68 | [M + 2H]/2 |
| 444 | 2639.05 | 2637.25 | 881.13 | [M + 3H]/3 |
| 445 | 2509.94 | 2508.21 | 837.77 | [M + 3H]/3 |
| 446 | 2597.02 | 2595.24 | 866.82 | [M + 3H]/3 |
| 447 | 2391.76 | 2390.11 | 798.33 | [M + 3H]/3 |
| 448 | 2640.04 | 2638.24 | 881.20 | [M + 3H]/3 |
| 449 | 2350.71 | 2349.09 | 784.67 | [M + 3H]/3 |
| 453 | 2575.03 | 2573.25 | 859.10 | [M + 3H]/3 |
| 454 | 2632.08 | 2630.27 | 878.10 | [M + 3H]/3 |
| 455 | 2582 | 2580.23 | 861.50 | [M + 3H]/3 |
| 458 | 2550.95 | 2549.20 | 851.00 | [M + 3H]/3 |
| 459 | 2520.88 | 2519.16 | 841.00 | [M + 3H]/3 |
| 460 | 2463.82 | 2462.13 | 1230.50 | [M − 2H]/2, |
| | | | | negative mode |
| 474 | 2551.93 | 2550.19 | 1276.50 | [M + 2H]/2 |
| 475 | 2521.86 | 2520.14 | 1261.50 | [M + 2H]/2 |
| 476 | 2464.81 | 2463.12 | 1233.00 | [M + 2H]/2 |
| 483 | 2598 | 2596.23 | 867.24 | [M + 3H]/3 |
| 484 | 2598.98 | 2597.21 | 867.47 | [M + 3H]/3 |
| 485 | 2623.05 | 2621.25 | 875.45 | [M + 3H]/3 |
| 486 | 2504.87 | 2503.15 | 1254.10 | [M + 2H]/2 |
| 487 | 2633.99 | 2632.19 | 879.45 | [M + 3H]/3 |
| 488 | 2640.04 | 2638.24 | 881.41 | [M + 3H]/3 |
| 489 | 2584.96 | 2583.21 | 862.71 | [M + 3H]/3 |
| 490 | 2598.98 | 2597.21 | 1301.05 | [M + 2H]/2 |
| 494 | 2597.01 | 2595.23 | 1297.10 | [M − 2H]/2, |
| | | | | negative mode |
| 495 | 2653.08 | 2651.27 | 885.10 | [M + 3H]/3 |
| 496 | 2711.12 | 2709.28 | 904.40 | [M + 3H]/3 |
| 510 | 2582 | 2580.23 | 861.40 | [M + 3H]/3 |
| 511 | 2596.03 | 2594.25 | 866.10 | [M + 3H]/3 |
| 512 | 2612.03 | 2610.24 | 871.50 | [M + 3H]/3 |
| 513 | 2638.06 | 2636.26 | 1317.60 | [M − 2H]/2, |
| | | | | negative mode |
| 514 | 2639.05 | 2637.25 | 880.40 | [M + 3H]/3 |
| 518 | 2831.29 | 2829.29 | 944.60 | [M + 3H]/3 |
| 519 | 2832.27 | 2830.27 | 1417.00 | [M + 2H]/2 |
| 520 | 2610.05 | 2608.26 | 1303.60 | [M − 2H]/2, |
| | | | | negative mode |
| 521 | 2598.04 | 2596.26 | 866.70 | [M + 3H]/3 |
| 522 | 2613.08 | 2611.29 | 871.40 | [M + 2H]/3 |
| 523 | 2569.99 | 2568.23 | 857.40 | [M + 3H]/3 |
| 526 | 2654.06 | 2652.25 | 885.69 | [M + 3H]/3 |
| 536 | 2596.03 | 2594.25 | 866.49 | [M + 3H]/3 |
| 537 | 3039.53 | 3037.49 | 1014.58 | [M + 3H]/3 |
| 538 | 3040.51 | 3038.47 | 1014.86 | [M + 3H]/3 |
| 540 | 2597.01 | 2595.23 | 867.12 | [M + 3H]/3 |
| 541 | 2639.05 | 2637.25 | 880.72 | [M + 3H]/3 |
| 542 | 2696.1 | 2694.28 | 899.70 | [M + 3H]/3 |
| 548 | 2380.73 | 2379.10 | 794.70 | [M + 3H]/3 |
| 549 | 2406.77 | 2405.11 | 803.24 | [M + 3H]/3 |
| 550 | 2535.89 | 2534.15 | 846.32 | [M + 3H]/3 |

### Example 2a. Affinity Determination by Surface Plasmon Resonance (SPR)

SPR studies were performed on the compounds disclosed herein using the following protocol.

Procedure: Binding affinities and binding kinetics of analytes were characterized with a single cycle kinetics method with Biacore 8K instrument. Biotinylated human 4lg B7-H3 was captured on a SA sensor chip with final level between 2100RU and 2400RU. Analytes were prepared with PBS buffer supplemented with 0.05% Surfactant P20 and 2% DMSO. Analytes were titrated from top concentration with four serial 1:3 dilutions. For interaction analysis, prepared analytes were injected over sensor chip with a flow rate of 50-100 μl/minute, an association time of 120 seconds, a dissociation time of 1000-3000 seconds, while maintaining a 25° C. or 37° C. flow cell temperature. Raw data was processed with standard procedures and was subsequently fitted with 1:1 binding model using Biacore Insight Evaluation 4.0. software.

Tables 7A and 7B below shows the K_{D} values obtained by the SPR assay for a group of selected compounds. In this Table, “A” represents K_{D}≤1.0 nM; “B” represents 1.0 nM<K_{D}≤ 10 nM; “C” represents 10 nM<K_{D}≤100 nM; “D” represents 100 nM<K_{D}≤300 nM.

TABLE 7A
SPR binding affinity of selected compounds to 4Ig B7-H3.

| | SPR | SPR |
|---|---|---|
| Cpd# | K_{D}_{_{—}}25° C. | K_{D}_{_{—}}37° C. |
| 1 | D | D |
| 5 | | C |
| 7 | | C |
| 24 | | C |
| 26 | C | C |
| 27 | B | B |
| 28 | B | B |
| 29 | | C |
| 31 | | D |
| 32 | B | C |
| 33 | | C |
| 34 | B | C |
| 35 | C | |
| 36 | | D |
| 52 | C | |
| 53 | | D |
| 54 | C | |
| 55 | B | |
| 56 | C | |
| 58 | B | |
| 59 | B | |
| 61 | B | |
| 62 | D | |
| 63 | B | |
| 65 | C | |
| 66 | C | |
| 67 | B | |
| 72 | A | |
| 73 | A | |
| 74 | B | |
| 75 | B | |
| 76 | A | |
| 77 | B | |
| 78 | B | |
| 79 | C | |
| 80 | C | |
| 81 | D | |
| 82 | D | |
| 85 | B | |
| 86 | C | |
| 87 | C | |
| 89 | C | |
| 92 | B | |
| 93 | C | |
| 94 | C | |
| 96 | C | |
| 102 | D | |
| 103 | C | |
| 116 | C | |
| 117 | C | |
| 119 | C | |
| 120 | C | |
| 121 | B | |
| 122 | C | |
| 123 | B | |
| 125 | D | |
| 126 | A | |
| 127 | D | |
| 128 | B | |
| 129 | D | |
| 131 | C | |
| 132 | B | |
| 133 | B | |
| 134 | C | |
| 135 | B | |
| 138 | B | |
| 139 | A | |
| 140 | B | |
| 141 | B | |
| 146 | A | |
| 147 | B | |
| 148 | A | |
| 149 | A | |
| 150 | A | |
| 151 | B | |
| 152 | A | |
| 153 | A | |
| 154 | A | |
| 155 | A | |
| 156 | C | |
| 157 | A | |
| 158 | A | |
| 159 | C | |
| 160 | A | |
| 161 | B | |
| 162 | A | |
| 163 | C | |
| 164 | A | |
| 165 | B | |
| 166 | C | |
| 167 | B | |
| 168 | D | |
| 170 | B | |
| 171 | A | |
| 172 | B | |
| 173 | A | |
| 174 | C | |
| 176 | B | |
| 178 | B | |
| 179 | A | |
| 180 | B | |
| 181 | B | |
| 184 | B | |
| 185 | B | |
| 186 | B | |
| 189 | D | |
| 190 | A | |
| 192 | B | |
| 193 | C | |
| 195 | C | |
| 198 | B | |
| 199 | B | |
| 200 | A | B |
| 205 | B | |
| 211 | C | |
| 212 | D | |
| 213 | D | |
| 215 | A | |
| 216 | A | |
| 217 | B | |
| 219 | B | |
| 220 | C | |
| 221 | D | |
| 223 | C | |
| 225 | C | |
| 227 | A | |
| 235 | B | |
| 236 | A | |
| 237 | B | |
| 240 | A | |
| 243 | C | |
| 244 | A | |
| 245 | B | |
| 246 | B | |
| 247 | B | |
| 248 | B | |
| 259 | A | B |
| 271 | A | |
| 272 | D | |
| 274 | A | |
| 275 | B | |
| 276 | B | |
| 277 | B | |
| 278 | B | |
| 280 | A | |
| 281 | B | |
| 282 | A | |
| 283 | C | |
| 284 | C | |
| 285 | B | |
| 286 | A | B |
| 287 | C | |
| 288 | A | |
| 291 | A | |
| 293 | C | |
| 294 | B | |
| 297 | D | |
| 303 | A | A |
| 304 | B | |
| 306 | C | |
| 307 | B | |
| 308 | A | |
| 310 | A | A |
| 311 | B | |
| 312 | B | |
| 313 | | A |
| 314 | | A |
| 315 | | B |
| 318 | | B |
| 319 | | B |
| 320 | | B |
| 321 | | C |
| 327 | | C |
| 331 | A | |
| 332 | A | |
| 333 | C | |
| 334 | C | |
| 335 | B | |
| 339 | | A |
| 340 | | A |
| 341 | | C |
| 347 | | A |
| 348 | | A |
| 349 | | C |
| 350 | | C |
| 352 | C | |
| 353 | D | |
| 359 | D | |
| 360 | B | |
| 361 | B | |
| 362 | A | |
| 364 | | B |
| 365 | | C |
| 366 | | A |
| 367 | | B |
| 368 | | A |
| 370 | | B |
| 371 | | A |
| 372 | | A |
| 373 | | B |
| 375 | | B |
| 377 | | C |
| 378 | | D |
| 386 | | B |
| 389 | | A |
| 391 | | B |
| 392 | | B |
| 393 | | B |
| 394 | | B |
| 395 | | B |
| 396 | | D |
| 397 | | C |
| 398 | | A |
| 404 | | B |
| 405 | | C |
| 427 | | A |
| 428 | | A |
| 429 | | A |
| 430 | | A |
| 431 | | B |
| 432 | | A |
| 433 | | A |
| 434 | | A |
| 435 | | A |
| 436 | | A |
| 437 | | A |
| 438 | | A |
| 439 | | A |
| 440 | | A |
| 441 | | A |
| 442 | | A |
| 443 | | A |
| 450 | | B |
| 451 | | B |
| 452 | | A |
| 456 | | A |
| 457 | | A |
| 462 | | A |
| 464 | | A |
| 465 | | A |
| 467 | | B |
| 468 | | A |
| 469 | | A |
| 470 | | A |
| 471 | | B |
| 472 | | B |
| 473 | | B |
| 477 | | B |
| 478 | | C |
| 479 | | A |
| 480 | | A |
| 481 | | A |
| 482 | | B |
| 492 | | A |
| 493 | | B |
| 497 | | A |
| 498 | | A |
| 499 | | A |
| 500 | | A |
| 501 | | A |
| 502 | | A |
| 503 | | A |
| 504 | | A |
| 505 | | A |
| 506 | | A |
| 507 | | A |
| 508 | | A |
| 509 | | A |
| 515 | | A |
| 516 | | A |
| 517 | | A |
| 524 | | A |
| 525 | | A |
| 552 | C | |

TABLE 7B
SPR binding affinity of selected compounds to 4Ig B7-H3.

| | SPR | SPR |
|---|---|---|
| Cpd# | K_{D}_{_{—}}25° C. | K_{D}_{_{—}}37° C. |
| 136 | | A |
| 143 | C | |
| 144 | C | |
| 145 | B | |
| 182 | A | |
| 183 | B | |
| 191 | B | |
| 201 | B | |
| 202 | B | |
| 203 | A | |
| 228 | B | |
| 229 | A | |
| 230 | A | |
| 232 | C | |
| 233 | C | |
| 249 | B | |
| 250 | C | |
| 251 | B | |
| 252 | B | |
| 253 | B | |
| 254 | B | |
| 255 | B | |
| 256 | B | |
| 257 | B | |
| 258 | B | |
| 260 | B | |
| 261 | B | |
| 262 | C | |
| 263 | C | |
| 264 | B | |
| 265 | C | |
| 267 | C | |
| 268 | B | |
| 269 | C | |
| 273 | C | |
| 279 | B | |
| 289 | B | |
| 290 | D | |
| 292 | B | |
| 295 | A | A |
| 301 | A | |
| 302 | D | |
| 305 | D | |
| 309 | B | |
| 322 | | C |
| 323 | | B |
| 324 | | C |
| 325 | | C |
| 326 | | C |
| 328 | | C |
| 330 | D | |
| 336 | | C |
| 337 | | B |
| 338 | | B |
| 342 | | C |
| 343 | | C |
| 344 | | B |
| 382 | | B |
| 383 | | A |
| 384 | | A |
| 385 | | A |
| 407 | | A |
| 408 | | A |
| 409 | | B |
| 410 | | A |
| 419 | | B |
| 420 | | B |
| 421 | | B |
| 422 | | B |
| 423 | | B |
| 424 | | B |
| 425 | | B |
| 444 | | A |
| 445 | | A |
| 446 | | A |
| 447 | | A |
| 448 | | A |
| 449 | | A |
| 453 | | A |
| 454 | | A |
| 455 | | A |
| 458 | | A |
| 459 | | A |
| 460 | | A |
| 474 | | B |
| 475 | | B |
| 476 | | A |
| 483 | | A |
| 484 | | A |
| 485 | | A |
| 486 | | A |
| 487 | | A |
| 488 | | A |
| 489 | | A |
| 490 | | A |
| 494 | | A |
| 495 | | A |
| 496 | | A |
| 510 | | A |
| 511 | | A |
| 512 | | A |
| 513 | | A |
| 514 | | A |
| 518 | | A |
| 519 | | A |
| 520 | | A |
| 521 | | A |
| 522 | | A |
| 523 | | A |
| 526 | | A |

TABLE 7C
SPR binding affinity of selected compounds to 2Ig B7-H3.

| Cpd# | 2Ig B7-H3_K_{D} (nM) at 25° C. | 2Ig B7-H3_K_{D} (nM) at 37° C. |
|---|---|---|
| 301 | 860 | |
| 408 | | 920 |
| 410 | | 820 |
| 268 | | 7000 |
| 344 | | 6200 |
| 475 | | >10000 |
| 494 | | 370 |
| 496 | | 526 |
| 519 | | 510 |
| 521 | | 260 |
| 523 | | 520 |
| 526 | | 380 |

### Example 2b. Cell Binding, Internalization, and Radiolabeling of Cyclic Peptides

General Procedure for Radiolabeling of Cyclic Peptides with [¹⁷⁷Lu]LuCl₃

[¹⁷⁷Lu]LuCl₃ was received from venders in HCl solution. For every mCi of [¹⁷⁷Lu]LuCl₃ added to the reaction vial (1.5 mL Eppendorf vial), ammonium acetate buffer (0.2 M, pH 4.9, 100 μL, containing 1% w/v ascorbic acid and 6% v/v ethanol) and peptide conjugate (1 nmol) was added. The pH of the solution was determined to be approximately 5 by using pH strips. The reaction vial was incubated at 80° C., 700 RPM for 17-20 minutes. A sample from the reaction was analyzed by RP-HPLC using an Agilent Infinity II 1260 HPLC to determine reaction completion and radiochemical purity. HPLC conditions: Waters XBridge BEH C18 Column, 130 Å, 3.5 μm, 4.6 mm×250 mm; mobile phase: Solvent A=water (with 0.1% formic acid), solvent B=acetonitrile (with 0.1% formic acid). Gradients: 25-45% B in 10 minutes, 45-65% B in 12 minutes, 65-90% in 6 minutes at a flow rate of 0.5 mL/minute. Required amounts of the product were formulated in 1% PBSA for cell studies.

Reagents and Materials:

| Reagent/Material | Manufacturer | Category Number |
|---|---|---|
| Fetal Bovine Serum | R&D Systems | S11195 |
| TrypLE Select Enzyme (1x) | Gibco | 12563029 |
| Versene (1X) | Thermo Fisher | 15040-066 |
| PBS pH 7.2 | Gibco | 10010001 |
| Bovine Serum Albumin | Sigma Aldrich | A9576 |
| Distilled Water | Gibco | 15230162 |
| PBS + 1% Bovine Serum Albumin | In House | NA |
| Sodium Hydroxide (NaOH) | Thermo Fisher | A4782902 |
| Glycine | Thermo Fisher | J61855-AP |
| Acid Wash (Glycine + Water) | In House | NA |
| 10% NaOH (NaOH + Water) | In House | NA |
| Trypan Blue | Gibco | 15250061 |
| EMEM | Gibco | 670086 |

### Instruments:

| Instrument | Manufacturer | Category Number |
|---|---|---|
| Biosafety Cabinet | Thermo Scientific | 1389-M |
| Incubator | Thermo Scientific | 381 |
| Gamma Counter | Perkin Elmer | 2470 |
| Dry Block Heating Shaker | Eppendorf | |
| Countess Cell Counter | Thermo Fisher | A49893 |
| Countess Cell Counting | Thermo Fisher | C10283 |
| Chamber Slides | | |
| Tissue Culture Treated | Falcon | 353112 |
| Flask 175 cm2 | | |
| Bio Lite 75 cm2 Flask | Thermo Fisher | 130190 |

### Method

### Cell Preparation

The study was conducted using Calu-6 cell line (Table 8)

Adherent cell studies: The cells were cultured in appropriate culture media (20 mL) in tissue culture treated T150 flasks at 37° C. and 5% CO2. Adherent cells were detached (60-70% confluent) using 5 mL Versene at 37° C. for 3 minutes. After confirming the viability using countess and count of detached cells using Trypan blue, the cells were centrifuged at 4° C. for 5 minutes (1,000 revolutions per minute). The cell pellet obtained was washed once with PBS and resuspended in 1% PBSA to obtain the desired cell concentration (5 million cells/mL).

TABLE 8
Cell line information.

| | | | Growth | Growth | Growth |
|---|---|---|---|---|---|
| Name | Source | Catalogue # | mode | Conditions | Media |
| Calu-6 | ATCC | HTB-56 | Adherent | 37° C.; | EMEM + |
| | | | | 5% CO2 | 10% FBS |

### Study Execution

Cell associated fraction: Cells were incubated with 300 μL (0.8 μCi) of the incubation buffer (containing radiolabeled peptide [¹⁷⁷Lu]Lu-test peptide) for 1 hour at 37 C (Table 9). Post incubation, the cells were pelleted and washed 2 times using cold PBS+1% BSA. The supernatant and washes were combined and counted together as “unbound fraction” in a gamma counter. The cells were then resuspended in 300 μL of 1% PBS+1% BSA and counted in the gamma counter as cell associated fraction (or bound fraction).

Net internalization: The resuspended cells in 300 μl of 1% PBSA and counted in the gamma counter as “bound fraction” were collected in 1.5 mL Eppendorf tube and pelleted. The pelleted cells were incubated with 300 μl of 50 mM Glycine pH=2.5 for 3 minutes and then pelleted discarding the supernatant. This process was repeated one more time. The pelleted cells were washed with 300 μl of cold 1% PBSA and then resuspended in 300 μl 0.3 M NaOH. This was counted in the gamma counter as “internalized fraction”.

TABLE 9
Summary of the experimental condition.

| Cell | Adherent/ | Dissociation | Cell no. | Binding | Incubation | Incubation | Thermomixer |
|---|---|---|---|---|---|---|---|
| Name | Suspension/Mixed | Method | (Million) | Media | Time | Temp | Speed |
| Calu-6 | Adherent | Versene | 5 | 1% | 1 hour | 37° C. | 700 rpm |
| | | | | PBSA | | | |

Calu-6 Cells are Human-Derived, Non-Engineered Lung Cancer Cell that Express B7-H3.

Tables 10A and 10B below show the cell assay data for a group of selected compounds. In these Tables, “A” represents %≤25; “B” represents 25<%≤50; “C” represents %>50.

TABLE 10A
Cell Data in Calu-6 cell line.

| | % cell associated | % net |
|---|---|---|
| Cpd# | fraction | internalization |
| 72 | C | A |
| 73 | C | A |
| 74 | B | A |
| 76 | C | A |
| 77 | C | A |
| 157 | B | A |
| 158 | C | B |
| 160 | B | A |
| 162 | B | A |
| 179 | B | A |
| 190 | C | B |
| 200 | C | A |
| 215 | C | A |
| 227 | C | B |
| 259 | B | A |
| 282 | B | A |
| 286 | C | A |
| 288 | B | A |
| 294 | A | A |
| 303 | C | B |
| 308 | C | B |
| 310 | C | B |
| 318 | B | A |
| 339 | C | B |
| 340 | C | A |
| 348 | B | A |
| 366 | B | A |
| 368 | B | A |
| 371 | B | A |
| 398 | B | A |
| 427 | C | B |
| 428 | B | A |
| 429 | B | A |
| 433 | C | B |
| 436 | B | A |
| 437 | B | A |
| 438 | B | A |
| 440 | B | A |
| 464 | C | A |
| 465 | B | A |
| 497 | C | A |
| 499 | C | A |
| 501 | C | B |

TABLE 10B
Cell Data in Calu-6 cell line.

| | % cell associated | % net |
|---|---|---|
| Cpd# | fraction | internalization |
| 182 | C | C |
| 203 | B | A |
| 230 | B | A |
| 252 | B | A |
| 253 | A | A |
| 256 | A | A |
| 338 | B | A |
| 383 | B | B |
| 384 | B | A |
| 385 | C | B |
| 407 | C | A |
| 408 | C | A |
| 410 | B | A |
| 444 | B | A |
| 455 | C | B |
| 494 | C | B |
| 495 | C | A |
| 496 | B | A |

### Example 2c: Ex Vivo Biodistribution

### General Protocols for Ex Vivo Biodistribution Studies

Mice were intravenously injected (via the lateral tail vein) with a bolus dose of the radiolabeled peptides for ex vivo biodistribution studies when tumor volumes were in range of 200-400 mm³ for xenograft mice and when age of the mice was 5-7 weeks for tumor naïve mice studies. To assess the amount of radionuclide injected into individual mice the syringe was assayed in a dose calibrator before and after injection for [¹⁷⁷Lu]Lu-peptide, then the injected dose was determined from the weight difference and concentration of the radionuclide solution.

Mice were euthanized at pre-determined time points and selected tissues were resected and collected into pre-weighed tubes. The tubes were then reweighed post resection, the difference giving the weight of each tissue. Radioactivity in each tissue was measured using a gamma counter. Counts were decay corrected to the time of injection and percentage of injected activity per gram (% IA/g) was calculated for each tissue based on the injected activity into each individual mouse. Injected activity was converted to counts based on the sensitivity of the gamma counter. The counts in tissue were then converted to the percentage of injected activity and this was divided by the mass of tissue to give % IA/g.

TABLE 10C
Radioactivity concentration (% IA/g) of tested compounds in different
organs in nude mice bearing Calu-6 tumors (n = 3) at 24
h post-injection. “A” represents 0 < % IA/g ≤
5 in tumor; “B” represents % IA/g > 5 in tumor;
“C” represents 0 < % IA/g ≤ 10 in kidney;
“D” represents % IA/g > 10 in kidney.

| | | tumor uptake | kidney uptake |
|---|---|---|---|
| Cpd# | Labeling | (% IA/g) | (% IA/g) |
| 338 | [¹⁷⁷Lu]Lu | A | C |
| 383 | [¹⁷⁷Lu]Lu | B | D |
| 384 | [¹⁷⁷Lu]Lu | A | D |
| 385 | [¹⁷⁷Lu]Lu | B | D |
| 407 | [¹⁷⁷Lu]Lu | A | C |
| 408 | [¹⁷⁷Lu]Lu | B | D |
| 410 | [¹⁷⁷Lu]Lu | B | D |
| 444 | [¹⁷⁷Lu]Lu | A | D |
| 455 | [¹⁷⁷Lu]Lu | A | D |
| 494 | [¹⁷⁷Lu]Lu | A | D |
| 496 | [¹⁷⁷Lu]Lu | A | C |
| 526 | [¹⁷⁷Lu]Lu | A | D |

### Example 3: [¹¹¹In] in-Labeled Peptide Development and Formulation

### I. [¹¹¹In]In-496 Radiolabeling and Formulation

Radiosynthesis of active pharmaceutical substance [¹¹¹In]In-496 in situ was conducted in 6 mL of 0.1 M sodium acetate reaction buffer containing 5 mg/mL ascorbic acid, with sequential addition of ˜60 μg (20 nmol) of peptide precursor Cpd 496 solution in ˜60 μL of sodium acetate buffer, and ˜60 mCi of [¹¹¹In]InCl3 solution in 0.05 M HCl. This reaction mixture was contained in a sealed 20 mL glass vial, heated at 70° C. for 20 minutes using a heating block followed by a cooling step at ambient room temperature for 10 minutes.

Immediately upon completion of the cooling step, [¹¹¹In]In-496 drug substance was formulated by the addition of a 6 mL of formulation buffer and unlabeled peptide solution directly into the 20 mL glass vial containing the reaction mixture. The vial was sealed and mixed.

### Contents and Concentrations of [¹¹¹In]In-496

| | |
|---|---|
| Volume (mL) | 8 |
| Radioactive Concentration (mCi/mL) | 5 |
| Total Activity (mCi) | 40 |
| Concentration of Cpd 496 (μg/mL) | 4.5-430 |
| Calculated Specific Activity (mCi/μg) | 1.1 |
| Concentration of Sodium Ascorbate (mg/mL) | 50 |
| Concentration of Ascorbic Acid (mg/mL) | 7.5 |
| Concentration of Tween-20 (%, w/v) | 0.05 |
| Concentration of Acetate (M) | 0.05 |
| pH | 5.2 |

In order to arrive at the final formulation, shown above, the buffer formulation was optimized with the goal of maintaining the RCP (>90%) throughout the target expiry of 144 hours. Initial conditions (Reaction 1) and optimized conditions (Reactions 2 and 3) are shown below.

| | |
|---|---|
| Reaction Number | 1 |
| Formulation | 30 mg/mL sodium ascorbate, |
| | 20 mg/mL ascorbic acid, |
| | 0.05% Polysorbate-20 w/v, |
| | 0.05M acetate |
| Dose Volume (mL) | 8 |
| Radioactive Conc. (mCi/mL) | 5 |
| Total activity (mCi) | 40 |
| Storage condition | 2-8° C. |
| RCP, End of Formulation (EOF) | 94.07% |
| RCP, 24-hour storage | 93.94% |
| RCP, 43-hour storage | 93.63% |
| RCP, 116-hour storage | 93.06% |
| RCP, 143-hour storage | 93.67% |
| RCP, 169-hour storage | 93.47% |

| | | |
|---|---|---|
| Reaction Number | 2 | 3 |

| | |
|---|---|
| Formulation | 50 mg/mL sodium ascorbate, |
| | 7.5 mg/mL ascorbic acid, |
| | 0.05% Tween ® 20 w/v, |
| | 0.05M acetate. |
| Dose Volume (mL) | 12 |
| Radioactive Conc. (mCi/mL) | 5 |

| | | |
|---|---|---|
| Total activity (mCi) | 59.7 | 60.4 |
| Storage condition | 2-8° C. | 2-8° C. |
| RCP, EOF | 96.04% | 96.38% |
| RCP, 24-hour storage | 95.55% | 95.73% |
| RCP, 48-hour storage | 95.82% | 95.98% |
| RCP, 72-hour storage | — | 96.44% |
| RCP, 96-hour storage | — | 95.71% |
| RCP, 120-hour storage | — | — |
| RCP, 144-hour storage | 95.63% | — |
| RCP, 168-hour storage | 95.82% | 96.04% |

The RCP and was determined using a RP-HPLC analysis method, described below. RCP was calculated as the proportion of the total radioactivity in the sample which is present as [¹¹¹In]In-496.

| | |
|---|---|
| System | Agilent 1260 Infinity II |
| Column | Waters XBridge C18; 3.5 μm, |
| | 4.6 × 250 mm |
| Column temperature | Ambient |
| UV wavelength (nm) | 220/280 |
| Radiodetection | Flow-RAM coupled to 1″ Nal PMT |
| Mobile Phase A | 0.1% (v/v) formic acid/water |
| Mobile Phase B | 0.1% (v/v) formic acid/acetonitrile |
| Flow rate (mL/minute) | 0.5 |
| Run time (minutes) | 35 |
| Retention time, | 16.1 |
| [¹¹¹In]In-496 (minutes) | |
| Retention time, unbound | 4.6 |
| [¹¹¹In]In (min) | |

Gradient Conditions

| | Mobile Phase | Mobile Phase |
|---|---|---|
| Time (min) | A (%) | B (%) |
| 00.00 | 75 | 25 |
| 24.00 | 35 | 65 |
| 28.00 | 10 | 90 |
| 29.00 | 10 | 90 |
| 30.00 | 75 | 25 |

### Example 4: High Specific Activity Radiolabeled Compounds for B7-H3

### I. High Specific Activity Production of [¹⁷⁷Lu]Lu-496

Representative reactions were carried out at a radioactivity scale range of 25-107 mCi in Eppendorf tubes and heating support using a small heater such as an orbital heater. Reaction volumes range from 650 μL to 1031 μL. The reaction buffer was an acetate buffer containing ascorbic acid as stabilizer. Reactions were heated at 80° C. setpoint for 20 minutes. Post heating and cooled reaction mixture was quenched with DTPA solution and injected into the analytical HPLC or Neptis semi-prep HPLC for purification. The specific purification method will vary based on the system used for purification and are described below. Fractions were collected into Formulation Buffer at the retention time of the [¹⁷⁷Lu]Lu-496 product. An aliquot of the formulated solution was analyzed by using RP-HPLC (Table 11). RCP (RCP) was reported based on HPLC analysis, and the specific activity was calculated based on the specific concentration and the concentration of peptide mass.

TABLE 11
Analytical HPLC method for RCP and peptide mass determination.

| System | Agilent 1260 Infinity II |
|---|---|
| Column | Waters XBridge C18; 3.5 μm, 4.6 × 250 mm |
| Column temperature | Ambient |
| UV wavelength (nm) | 220/280 |
| Radiodetection | Flow-RAM coupled to 1.5″ Nal PMT |
| Mobile Phase A | 0.1% (v/v) formic acid/water |
| Mobile Phase B | 0.1% (v/v) formic acid/acetonitrile |
| Flow rate (mL/minute) | 0.5 |
| Run time (min) | 35 |

Gradient Conditions

| Time (min) | Mobile Phase A (%) | Mobile Phase B (%) |
|---|---|---|
| 00.00 | 75 | 25 |
| 24.00 | 35 | 65 |
| 28.00 | 10 | 90 |
| 29.00 | 10 | 90 |
| 30.00 | 75 | 25 |

II. Radiolabeling and Purification with Analytical HPLC

To a 1.5 mL Eppendorf vial, 0.6 mL of 0.1 M Sodium acetate buffer containing 5 mg/mL ascorbic acid, 60 μL of ethanol, 223 μL of Compound 496 solution (0.298 nmol/μL) and 103 μL of Lu-177 chloride solution were added sequentially. The specific activity was 1.6 mCi/nmol for radiolabeling. The vial was assayed by a dose calibrator with Lu-177 calibration number and the activity was 106.8 mCi. The reaction vial was heated at 80° C. setpoint for 20 minutes. After the incubation was finished, the reaction was quenched with 30 μL of 10 mg/mL DTPA solution. 900 μL of the mixture (91.2 mCi) was injected into HPLC for purification with the purification method below.

Purification method: Waters XSelect CSH C18 4.6×150 mm, 3.5 μm; Mobile phase: 70% 1.5 mM NH4OAc in water, and 30% ethanol. Isocratic condition. Flow rate=0.5 mL/minute. Column temp=50° C. Run time=35 minutes. Flow-RAM coupled to 1″ Nal PMT was used for detection of the radioactive product

Fractions were collected at 18:54-20:00 and 20:00-21:00 and the activities were 74.9 mCi and 2.15 mCi, respectively. Fraction 1 was diluted with formulation buffer to the final volume of 5 mL. The formulation buffer consisted of 75 mg/mL sodium acetate, 7.5 mg/mL ascorbic acid, 0.75% Tween 20, and 0.75 mg/mL DTPA in water. The concentration of the diluted Fraction 1 was 14.98 mCi/mL. The final drug product formulation contained 50 mg/mL sodium ascorbate, 5 mg/mL ascorbic acid, 0.05% tween-20 and 0.05 mg/mL DTPA. The RCP was 96.36% after formulation, and the specific activity was measured at 19.0 mCi/nmol. The sample was stored at 2-8° C. The RCP was 95.69% after 2-day storage. The results are summarized in the following tables.

TABLE 12

| Radiolabeled Compound | HSA [¹⁷⁷Lu]Lu-496 |
|---|---|
| RCP | 96.4% |
| Radioactive Concentration (mCi/mL) | 8.69 |
| Drug Mass Concentration (μg/mL) | 1.2 |
| Total activity (mCi) | 40.5 |
| Total volume (mL) | 4.66 |
| Storage condition | 2-8° C. |

TABLE 13

| | Sodium ascorbate 50 mg/mL, |
|---|---|
| | Ascorbic acid 5 mg/mL, |
| | Tween-20 0.05%, |
| HSA [¹⁷⁷Lu]Lu-496 Formulation | Ethanol content 10% |
| RCP, EOF after purification (HPLC, %) | 96.36 |
| ¹⁷⁷Lu incorporation, | 99.72 |
| EOF after purification (HPLC, %) | |
| RCP, 2 day storage 2-8° C. (HPLC, %) | 95.69 |
| ¹⁷⁷Lu incorporation, | 99.44 |
| 2 day storage 2-8° C. (HPLC, %) | |

III. Purification with Neptis Semi-Prep HPLC

To a 1.5 mL Eppendorf vial, 0.2 mL of 0.1 M Sodium acetate buffer containing 5 mg/mL ascorbic acid, 0.6 mL 0.1 M Sodium acetate buffer, 50 μL of ethanol, 94 μL of Compound 496 solution (0.424 nmol/μL) and 87 μL of Lu-177 chloride solution were added sequentially. The vial was assayed by a dose calibrator with Lu-177 calibration number and the activity was 70.1 mCi. The reaction vial was heated at 80° C. for 15 minutes. After the incubation was finished, the reaction was quenched with 10 μL of 10 mg/mL DTPA solution. All the mixture (65 mCi) was injected by the automated Neptis system equipped with an HPLC system for purification.

Purification method: Waters XSelect CSH C18 4.6×250 mm, 5 μm; Mobile phase: 66% 5 mM ammonium acetate; 34% ethanol; isocratic, Column temperature=ambient temperature. Flow rate=1 mL/minute. Flow-RAM coupled to 1″ Nal PMT was used for detection of the radioactive product.

Fraction was collected between 19:30-22:00 for 2.5-minute collection. The activity collected was 42.3 mCi. The product collection vial was prefilled with 1 mL of stabilization buffer (100 mg/mL sodium ascorbate, 10 mg/mL ascorbic acid, 0.1% tween-20). After collection, 5 mL of formulation buffer (50 mg/mL sodium ascorbate, 5 mg/mL ascorbic acid, 0.05% tween-20) was added to the collection vial to yield a final volume at 8.5 mL. The RCP was 98.97%.

IV. Efficacy Study with [¹⁷⁷Lu]Lu-496 in a CDX Mouse Model (NCI-H1650 and SHP77)

The objective of this study was to compare the efficacy of RSA and HSA [¹⁷⁷Lu]Lu-496 drug products in a tumor model of human lung small cell lung cancer using SHP77 cells, which exhibit low receptor expression (˜27k B7-H3 receptors/cell), and in NCI-H1650 cells, which exhibit relatively high receptor expression (˜212k B7-H3 receptors/cell). A cell line-derived xenograft (CDX) mouse model is a research tool that involves implanting human tumor cells into an immunodeficient mouse to evaluate the efficacy of a cancer therapy. The chosen cell lines are good models to explore efficacy as they represent low end and high end of B7-H3 expression for CDX models.

All mice were sorted into study groups based on caliper estimation of tumor burden. The mice were distributed to ensure that the mean tumor burden for all groups was within 10% of the overall mean tumor burden for the study population. Particular details of the study are shown in the following table.

TABLE 14

| | | | | | | Treatment |
|---|---|---|---|---|---|---|
| Group | N | Treatment | Dose | ROA | Regimen | (days) |
| 1 | 10 | Vehicle | 200 μL/injection | IV | QDx1 | 13 |
| 2 | 10 | [1⁷⁷Lu] Lu-496 (RSA) | 1400 μCi/animal | IV | QDx1 | 13 |
| | | (1.4 mCi at | | | | |
| | | 200 uL/injection) | | | | |
| 3 | 10 | [¹⁷⁷Lu] Lu-496 (HSA) | 1400 μCi/animal | IV | QDx1 | 13 |
| | | (1.4 mCi at | | | | |
| | | 200 uL/injection) | | | | |
| 4 | 10 | [¹⁷⁷Lu] Lu-496 (RSA) | 700 μCi/animal | IV | QDx1 | 13 |
| | | (0.7 mCi at | | | | |
| | | 100 uL/injection) | | | | |
| 5 | 10 | [¹⁷⁷Lu] Lu-496 (HSA) | 700 μCi/animal | IV | QDx1 | 13 |
| | | (0.7 mCi at | | | | |
| | | 100 uL/injection) | | | | |

The RSA drug products were characterized as having a specific molar activity of about 1.4 mCi/nmol, whereas the HSA drug products were characterized as having a specific molar activity of about 15.5 mCi/nmol (Table 15).

TABLE 15

| | Specific activity | Total mass | % labeled/% |
|---|---|---|---|
| Dose (mCi) | (mCi/nmol) | dose (ug) | unlabeled peptide |
| 1.4 mCi | 15.5 | 0.19 | 80%/20% |
| (HSA) | | | |
| 1.4 mCi | 1.37 | 2.1 | 7.1%/92.9% |
| (RSA) | | | |
| 0.7 mCi | 15.5 | 0.095 | 80%/20% |
| (HSA) | | | |
| 0.7 mCi | 1.37 | 1.05 | 7.1%/92.9% |
| (RSA) | | | |

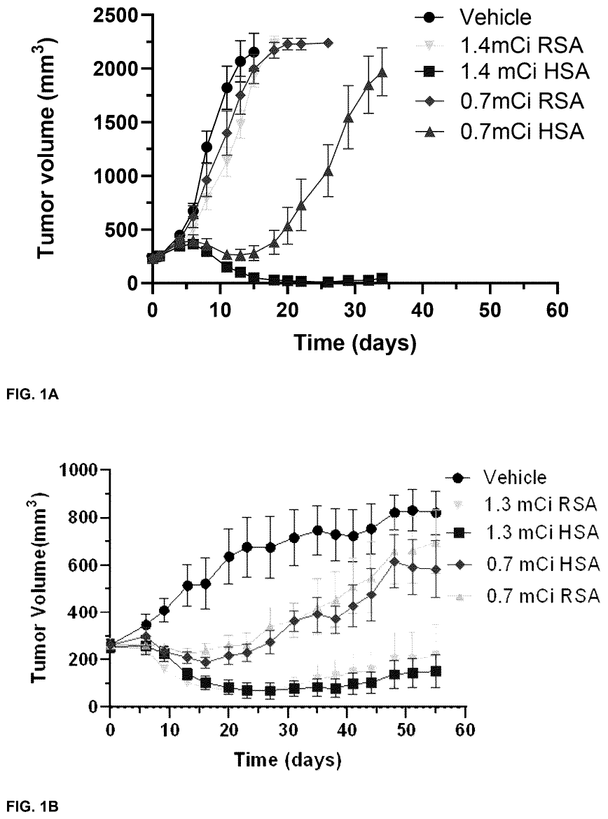

Treatment of animals having SHP77 tumors with [177Lu]Lu-496 (HSA) at 1400 μCi/animal (1.4 mCi/animal) or 700 μCi/animal (0.7 mCi/animal) (Groups 3 and 5) produced dose-dependent anti-cancer activity. High dose treatment in Group 3 produced a Day 24 regression value of 28% and 80.0% incidences of complete regressions and tumor-free survivors. Low dose treatment in Group 5 produced a Day 24 median AT/AC value of 1%, an increase in time to progression of 163.6%, and 10.0% incidence of complete regressions. Treatment with [¹⁷⁷Lu]Lu-496 (RSA) at 1400 μCi/animal or 700 μCi/animal (Groups 2 and 4) produced median .T/.C values of 46% and 68% and ITP values of 27.2% and 9.0%, respectively. See, FIG. 1A.

As shown in FIG. 1B, equivalent efficacy was observed with HSA vs RSA approaches for higher the expressing model (NCI-H1650).

### Example 5: HSA [²²⁵Ac]Ac-496

### I. Small-Scale High Specific Activity Production of [²²⁵Ac]Ac-496

[²²⁵Ac]Ac source: Actinium-225 nitrate dissolved in 0.04 M hydrochloric acid to a radioactive concentration of 2-20 mCi/mL.

Reaction Buffer: 1 M sodium acetate pH 5-6 with a variable concentration of ascorbic acid such that the total reaction volume contains an ascorbic acid concentration of 5 mg/mL.

Peptide Solution: Compound 496 dissolved in 0.1 M sodium acetate pH 5-6 buffer to a concentration of 0.592-0.690 nmol/μL.

Formulation Buffer: 0.1 M ammonium acetate buffer pH 5.5-6.0 containing sodium ascorbate, L-methionine, and Tween-20 such that the concentrations in the radiolabeled product formulation are 50 mg/mL, 23.6 mg/mL, and 0.05% w/v respectively.

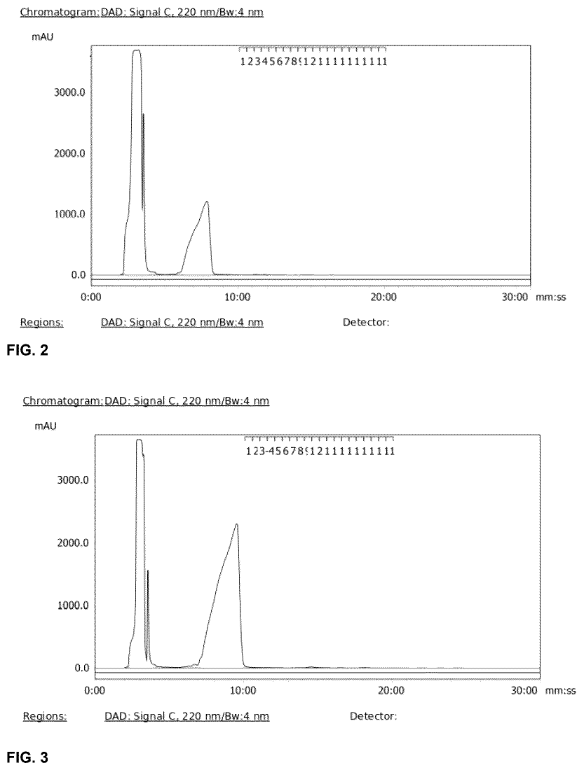

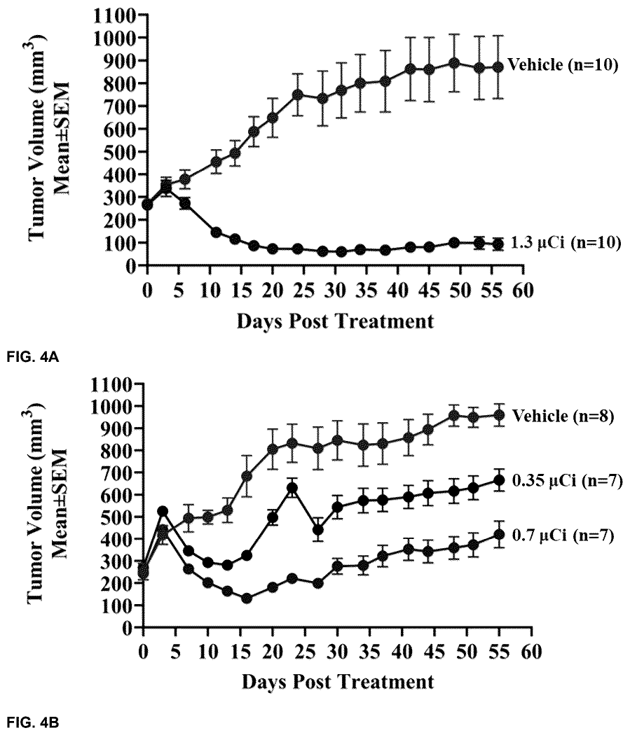

Procedure (Table 16 through Table 20): Ac-225 source was added to a 1.5 mL Eppendorf Lo-Bind microcentrifuge tube. Reaction Buffer was added to the vial at a volume of 0.5-0.6 times the volume of Ac-225 source added. Ethanol was added to the vial such that the concentration in the total reaction volume is 9.4-10.0% v/v. Peptide Solution was added to the vial such that the targeted specific molar activity was 4.9 μCi/nmol. The reaction mixture was incubated at 90° C. for 15 minutes on a USA Scientific Mixer HC at 700 RPM. The reaction was then allowed to briefly cool before RP-HPLC purification. The reaction was transferred to an HPLC vial containing 5 μL of 0.05 mg/mL DTPA in a 0.1 M ammonium acetate buffer pH 5.5. The reaction vial was rinsed with an aliquot of Formulation Buffer and transferred to the HPLC vial such that the total volume in the HPLC vial was ˜110 μL. The vial contents were injected onto the HPLC. HPLC eluate was collected in 0.5 minute fractions between 10 and 20 minutes into vials that were preloaded with Formulation Buffer. The collected fractions were assayed for activity and the fractions containing the desired radiolabeled product identified (FIG. 2 and FIG. 3). These fractions were analyzed via RP-HPLC and iTLC for RCP. The radiolabeled product was then stored at 2-8° C.

TABLE 16
Reaction Summaries.

| | Run #1 | Run #2 |
|---|---|---|
| Ac-225 Activity (μCi) | 70 | 200 |
| Total Reaction Volume (μL) | 79.5 | 84.1 |
| Reaction Sodium Acetate Concentration (M) | 0.23 | 0.14 |
| Reaction Peptide Concentration (nmol/μL) | 0.18 | 0.49 |
| Reaction Radioactivity Concentration (μCi/μL) | 0.88 | 2.38 |
| Targeted Specific Molar Activity (μCi/nmol) | 4.9 | 4.9 |
| Isotope Incorporation via iTLC (%) | 99.25 | >99 |

TABLE 17
Results Summaries.

| ELN | Run #1 | Run #2 |
|---|---|---|
| Product Activity (μCi) | 44 | 156 |
| Sodium Ascorbate (mg/mL) | 50 | 50 |
| Methionine (mg/mL) | 23.6 | 23.6 |
| Tween-20 (% w/v) | 0.05 | 0.05 |
| Ethanol (% v/v) | 7.5 | 7.5 |
| Ammonium Acetate (M) | 0.08 | 0.08 |
| Product Radioactive Concentration (μCi/μL) | 0.04 | 0.05 |
| Radioactive Yield (%) | 63 | 78 |
| RCP via RP-HPLC (%) | 95.00 | 96.16 |
| RCP via iTLC (%) | 99.80 | >99 |
| 24 hr - RCP via RP-HPLC (%) | 94.60 | N/A |
| 24 hr - RCP via iTLC (%) | 99.90 | N/A |

TABLE 18
iTLC Conditions.

| | |
|---|---|
| Mobile phase | 50 mM EDTA; pH 5.4 |
| Stationary Phase | Agilent Silica Gel Impregnated |
| | Microfiber Paper (p/n: SGI0001) |
| Radiodetection | Eckert & Ziegler AR2000¹ |

¹Post-development, iTLC papers are stored until secular equilibrium between Ac-225 and its daughter radioactive isotopes is achieved (>6 hours). They are then scanned using the AR2000.

TABLE 19
Analytical RP-HPLC Conditions.

| | |
|---|---|
| System | Analytical HPLC (HPLC04) |
| Mobile phase A | 0.1% Formic acid in water |
| Mobile phase B | 0.1% Formic acid in acetonitrile |
| Column | Waters XBridge C18; 3.5 μm, |
| | 4.6 × 250 mm (p/n: 186003943) |
| Flow rate (mL/minute) | 0.5 |
| Running conditions | Gradient - 25%-75% B, 0-23 min |
| | 90% B, 23-28 min |
| Radiodetection | Perkin Elmer Wizard2 2470 Gamma Counter¹ |

¹Fractions were stored until secular equilibrium between Ac-225 and its daughter radioactive isotopes was achieved (>6 hours). Fractions were then analyzed using the gamma counter and a chromatogram was reconstructed from the CPM values as a function of time.

TABLE 20
Purification RP-HPLC Conditions.

| System | Analytical HPLC (HPLC01) |
|---|---|
| Mobile phase A | 70% 1.5 mM ammonium acetate |
| Mobile phase B | 30% ethanol |
| Column | Waters XSelect CSH C18; 3.5 μm, |
| | 4.6 × 150 mm (p/n: 186006957) |
| Column Temp (° C.) | 50 |
| Flow rate (mL/minute) | 0.5 |

### II. Large-Scale High Specific Activity Production of [²²⁵Ac]Ac-496

[²²⁵Ac]Ac source: Actinium-225 nitrate dissolved in 0.04 M hydrochloric acid to a radioactive concentration of 1 mCi/mL.

Reaction Buffer: 1 M sodium acetate pH 5-6 with a variable concentration of ascorbic acid such that the total reaction volume contains an ascorbic acid concentration of 5 mg/mL.

Peptide Solution: Compound 496 dissolved in 0.1 M sodium acetate pH 5-6 buffer to a concentration of 0.665 nmol/μL.

Formulation Buffer: 0.1 M ammonium acetate buffer pH 5.5-6.0 containing sodium ascorbate, L-methionine, and Tween-20 such that the concentrations in the radiolabeled product formulation are 50 mg/mL, 23.6 mg/mL, and 0.05% w/v respectively.

Procedure (Table 21 through Table 23): Ac-225 source was added to a 10 mL ALK vial. Reaction Buffer was added to the vial such that the volume added was 0.5 times the volume of Ac-225 source added. Ethanol was added to the vial such that the concentration in the total reaction volume was 9-10% v/v. Peptide Solution was added to the vial such that the targeted specific molar activity was ˜5 μCi/nmol. The vial was sealed and crimped and placed into the Neptis heater. The vial was incubated at 90° C. for 12 minutes and then cooled with compressed air to 40° C. prior to purification. The reaction mixture was pulled from the reaction vial via a syringe driver. The vial was then washed with a buffered DTPA solution which was subsequently drawn into the syringe and mixed with the reaction mixture. The syringe then pushed the reaction mixture into the HPLC injection loop to begin purification. A 1″ Na/l probe was used to detect radioactivity in the eluate post-column and the radiolabeled product was identified by radioactive signal. Product collection was triggered by the operator. During product collection, flow was diverted from waste into a pre-loaded 50 mL ALK vial containing 26.25 mL of Formulation Buffer. The product was then analyzed via RP-HPLC and iTLC for RCP. See above section for methods. The radiolabeled product was stored at 2-8° C.

TABLE 21
Neptis Reaction Summary.

| | |
|---|---|
| Ac-225 Activity (μCi) | 1598 |
| Total Reaction Volume (μL) | 3182 |
| Reaction Sodium Acetate Concentration (M) | 0.27 |
| Reaction Peptide Concentration (nmol/μL) | 0.10 |
| Reaction Radioactivity Concentration (μCi/μL) | 0.50 |
| Targeted Specific Molar Activity (μCi/nmol) | 5.0 |
| Isotope Incorporation via iTLC (%) | 97.85 |

TABLE 22
Neptis Results Summary.

| | |
|---|---|
| Product Activity (μCi) | 1320 |
| Sodium Ascorbate (mg/mL) | 50.0 |
| Methionine (mg/mL) | 23.6 |
| Tween-20 (% w/v) | 0.05 |
| Ethanol (% v/v) | 8.75 |
| Ammonium Acetate (M) | 0.076 |
| Product Radioactive Concentration (μCi/μL) | 0.038 |
| Radioactive Yield (%) | 83 |
| RCP via RP-HPLC (%) | 97.14 |
| RCP via iTLC (%) | 99.88 |
| 48 hr - RCP via RP-HPLC (%) | 96.89 |
| 48 hr - RCP via iTLC (%) | 99.80 |
| 192 hr - RCP via RP-HPLC (%) | 95.15 |
| 192 hr - RCP via iTLC (%) | 99.21 |

TABLE 23
Neptis Purification Conditions.

| | |
|---|---|
| Mobile phase | 3.25 mM ammonium acetate, 35% (v/v) ethanol |
| Column | Waters XSelect Peptide CSH C18; 130 Å, |
| | 5 μm, 10 × 250 mm (p/n: 1806008267) |
| Column Temp (° C.) | Uncontrolled Ambient |
| Flow rate (mL/minute) | 3.5 |
| Running conditions | Isocratic |
| Radiodetection | Flow-RAM with 1″ Na/I PMT |

III. Stability Comparison versus Regular Specific Activity [²²⁵Ac]Ac-496

Radiolabeling in this example proceeded as described in the above section, but without purification (Table 24 and Table 25). The reaction mixture was split into two equivalent formulations of differing activity and radioactive concentration. Ethanol was introduced into the final formulations by way of the formulation buffer. Formulations were stored in a 50 mL ALK vial and in the 10 mL ALK vial used for the reaction. Samples were then taken from each formulation for RP-HPLC and iTLC analysis. The two formulations were stored at 2-8° C.

TABLE 24
Reaction Summary - Regular Specific Activity - 5 μCi/nmol.

| | |
|---|---|
| Ac-225 Activity (μCi) | 2040 |
| Total Reaction Volume (μL) | 4052 |
| Reaction Sodium Acetate Concentration (M) | 0.26 |
| Reaction Peptide Concentration (nmol/μL) | 0.10 |
| Reaction Radioactivity Concentration (μCi/μL) | 0.50 |
| Targeted Specific Molar Activity (μCi/nmol) | 5.1 |

TABLE 25
Results Summaries - Regular Specific Activity - 5 μCi/nmol.

| | Run #1 | Run #2 |
|---|---|---|
| Product Radioactive | 0.04 | 0.11 |
| Concentration (μCi/μL) | | |
| Product Activity (μCi) | 1278 | 735 |
| Ascorbic Acid (mg/mL) | 0.40 | 1.1 |
| Sodium Ascorbate (mg/mL) | 50.1 | 49.4 |
| Methionine (mg/mL) | 23.7 | 23.3 |
| Tween-20 (% w/v) | 0.05 | 0.05 |
| Ethanol (% v/v) | 8.75 | 8.77 |
| Ammonium Acetate (M) | 0.079 | 0.079 |
| RCP via RP-HPLC (%) | 95.95 | 96.07 |
| RCP via iTLC (%) | 98.29 | 98.98 |
| 48 hr - RCP via RP-HPLC (%) | 95.21 | 95.14 |
| 48 hr - RCP via iTLC (%) | 98.31 | 98.25 |
| 192 hr - RCP via RP-HPLC (%) | 93.53 | 91.80 |
| 192 hr - RCP via iTLC (%) | N/A | N/A |

### Example 6: X-Ray Crystal Structure of an Exemplary Compound with 4lg-B7-H3

The x-ray crystal structure of an exemplary compound of the disclosure bound to 4lg-B7-H3 was determined. Suitable constructs for B⁷H3 expression had been previously established by PROTEROS. Expression of B⁷H3 was performed according to previously established protocols. A purification protocol was established and homogeneous protein was produced in preparative amounts. The protein was purified comprising affinity and gel filtration chromatography steps. This procedure yielded homogenous protein with a purity greater 95% as judged from Coomassie stained SDS-PAGE. A 4lg-B7-H3 sequence from amino acid 29-456 was employed. The following sequence corresponds to amino acids 1-534 of 4lg-B7-H3:

(SEQ ID NO: 553)
MLRRRGSPGMGVHVGAALGALWFCLTGALEVQVPEDPVVALVGTDATL
CCSFSPEPGFSLAQLNLIWQLTDTKQLVHSFAEGQDQGSAYANRTALF
PDLLAQGNASLRLQRVRVADEGSFTCFVSIRDFGSAAVSLQVAAPYDK
PSMTLEPNKDLRPGDTVTITCSSYQGYPEAEVFWQDGQGVPLTGNVTT
SQMANEQGLFDVHSILRVVLGANGTYSCLVRNPVLQQDAHSSVTITPQ
RSPTGAVEVQVPEDPVVALVGTDATLRCSFSPEPGFSLAQLNLIWQLT
DTKQLVHSFTEGRDQGSAYANRTALFPDLLAQGNASLRLQRVRVADEG
SFTCFVSIRDFGSAAVSLQVAAPYSKPSMTLEPNKDLRPGDTVTITCS
SYRGYPEAEVFWQDGQGVPLTGNVTTSQMANEQGLFDVHSVLRVVLGA
NGTYSCLVRNPVLQQDAHGSVTITGQPMTFPPEALWVTVGLSVCLIAL
LVALAFVCWRKIKQSCEEENAGAEDQDGEGEGSKTALQPLKHSDSKED
DGQEIA.

### Crystallization

The purified protein was used in crystallization trials employing both, a standard screen with approximately 1200 different conditions, as well as crystallization conditions identified using literature data. Conditions initially obtained have been optimized using standard strategies, systematically varying parameters critically influencing crystallization, such as temperature, protein concentration, drop ratio, and others. These conditions were also refined by systematically varying pH or precipitant concentrations.

### Data Collection and Processing

A cryo-protocol was established using PROTEROS Standard Protocols. Crystals have been flash-frozen and measured at a temperature of 100 K. The X-ray diffraction data have been collected from complex crystals of 4lg-B7-H3 with the ligand of Cpd #200 at the SWISS LIGHT SOURCE (SLS, Villigen, Switzerland) using cryogenic conditions. The crystals belong to space group P 61 2 2. Data were processed using the programs autoPROC, XDS and autoPROC, AIMLESS.

TABLE 26
Data collection and processing statistics for Cpd #200.

| Ligand | Cpd #200 |
|---|---|
| X-ray source | PXII/X₁₀SA (SLS¹) |
| Wavelength [Å] | 1.0000 |
| Detector | Dectris EIGER₂ Si 16M |
| Temperature [K] | 100 |
| Space group | P 6₁ 2 2 |
| Cell: a; b; c; [Å] | 94.72; 94.72; 304.95 |
| α; β; γ; [°] | 90.0; 90.0; 120.0 |
| Resolution [Å] | 2.98 (3.28-2.98) |
| Unique Reflections | 11895 (595) |
| Multiplicity | 23.6 (23.3) |
| Spherical completeness [%] | 68.5 (14.3) |
| Ellipsoidal completeness [%] | 94.6 (77.8) |
| Rₚᵢₘ [%]⁶ | 2.9 (53.6) |
| R_{sym} [%]³ | 13.6 (257.2) |
| Rₘₑₐₛ [%]⁴ | 14.0 (262.9) |
| CC1/2 [%] | 99.40 (68.50) |
| Means(I)/sd⁵ | 14.1 (1.7) |

¹ SWISS LIGHT SOURCE (SLS, Villigen, Switzerland)
² Values in parenthesis refer to the highest resolution bin.
³ $Rsym = \frac{{\sum }_{h} {\sum }_{i}^{{n}_{h}} \vert {\hat{I}}_{h} - {I}_{h , i} \vert}{{\sum }_{h} {\sum }_{i}^{{n}_{h}} {I}_{h , j}} with {\hat{I}}_{h} = \frac{1}{{n}_{h}} {\sum }_{i}^{{n}_{h}} {I}_{h , i}$ where I_{h,i} is the intensity value of the ith measurement of h
⁴ $Rmeas = \frac{{\sum }_{h} \sqrt{\frac{{n}_{h}}{{n}_{h} - 1}} {\sum }_{i}^{{n}_{h}} \vert {\hat{I}}_{h} - {I}_{h , i} \vert}{{\sum }_{h} {\sum }_{i}^{{n}_{h}} {I}_{h , i}} with {\hat{I}}_{h} = \frac{1}{{n}_{h}} {\sum }_{i}^{{n}_{h}} {I}_{h , i}$ where I_{h,i} is the intensity value of the ith measurement of h
⁵calculated from independent reflections
₆Precision-indicating $Rpim = \frac{{\sum }_{h} \sqrt{1 / ( N - 1 )} \vert {I}_{hl} - < {I}_{h} > \vert}{{\sum }_{h} \vert < {I}_{h} >}$

### Structure Modelling and Refinement

The phase information necessary to determine and analyze the structure was obtained by molecular replacement.

Subsequent model building and refinement was performed according to standard protocols with COOT and the software package CCP4, respectively. For the calculation of the free R-factor, a measure to cross-validate the correctness of the final model, about 4.8% of measured reflections were excluded from the refinement procedure (Table 27).

The ligand parameterization and generation of the corresponding library files were carried out with CORINA.

The water model was built with the “Find waters”-algorithm of COOT by putting water molecules in peaks of the Fₒ-F_{c} map contoured at 3.0 followed by refinement with REFMAC5 and checking all waters with the validation tool of COOT. The criteria for the list of suspicious waters were: B-factor greater 80 Å², 2Fₒ-F_{c} map less than 1.2 σ, distance to closest contact less than 2.3 Å or more than 3.5 Å. The suspicious water molecules and those in the ligand binding site (distance to ligand less than 10 Å) were checked manually.

The Ramachandran Plot of the final model calculated with Molprobity shows 89.10% of all residues in the favored region and 9.98% in the allowed region. Molprobity is described in further detail in Chen et al. (Acta Crystallogr D Biol Crystallogr. 2010. 66(Pt 1): 12-21), incorporated herein by reference. The residues Ile126(A), Arg241(A), Pro243(A), and Pro274(A) are outliers in the Ramachandran Plot. They are either defined by the electron density or could not be modelled in another sensible conformation. Statistics of the final structure and the refinement process are listed in Table 27.

TABLE 27
Refinement statistics for Cpd #200.

| Ligand | Cpd #200 |
|---|---|
| Resolution [Å] | 72.34-2.98 |
| Number of reflections (working/test) | 11326/569 |
| R_{cryst} [%] | 25.5 |
| R_{free} [%]² | 33.6 |
| Total number of atoms: | |
| protein | 3235 |
| Water | — |
| Ligand | 164 |
| N-acetyl-D-glucosamine | 56 |
| Average B-factors | |
| Protein | 119.1 |
| Other atoms | 142.3 |
| Deviation from ideal geometry:³ | |
| Bond lengths [Å] | 0.004 |
| Bond angles [°] | 1.50 |
| Bonded B's [Å²]⁴ | 5.5 |
| Ramachandran plot:⁵ | |
| Favored [%] | 89.10 |
| Allowed [%] | 9.98 |
| Outliers [%] | 0.93 |
| Molprobity score⁵ | 1.75 |
| Molprobity clashscore⁵ | 1.48 |

¹ Values as defined in REFMAC₅, without sigma cut-off
²Test-set contains 4.8% of measured reflections
³Root mean square deviations from geometric target values
⁴Calculated with MOLEMAN
⁵Calculated with Molprobity

The structure of the extracellular domain of 4lg-B7-H3 which spans residues 29-456, as annotated in UniProt Q5ZPR3, in complex with the ligand of Cpd #200 contains tandemly repeated immunoglobulin-like V and C domains (lg-like V-type 1, Ig-like C₂-type 1, Ig-like V-type 2 and Ig-like C₂-type 2). The ligand is bound between the Ig-like C₂-type 1 and Ig-like V-type 2 domains.

There is one monomer in the asymmetric unit and the model comprises residues Leu29 to Thr456. Some short loop regions are not fully defined by electron density and have thus not been included in the model (Table 27).

The amino acid residues forming the ligand binding site and the ligand are well defined in the electron density map. The interpreted X-ray diffraction data show a clear binding mode as well as orientation and conformation of the ligand bound to its binding site.

Based on a distance of less than 3.5 Å of the donor and acceptor atoms, eleven specific hydrogen bonds of Cpd #200 were identified. The following amino acids demonstrated a single hydrogen bond: D154, Q286, K291, M147, T238, and T290. S234 demonstrated two hydrogen bonds. T236 demonstrated three hydrogen bonds. Hydrogen bonds were identified with the program COOT and confirmed by visual inspection.

The following residues can be found in the vicinity of the ligand with a maximum distance of 3.9 Å: K144, M147, D154, L155, S234, T236, T238, N282, Q286, T290, K291, and L293.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A cyclic peptide of Formula I: 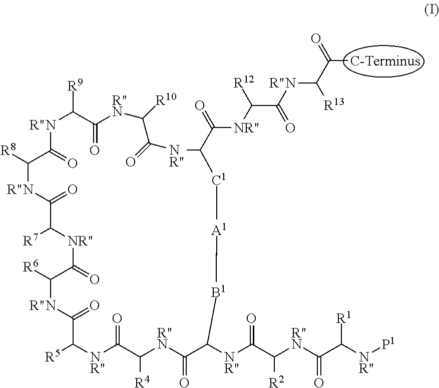
or a pharmaceutically acceptable salt thereof,
wherein:
the C-Terminus is selected from R, N(R″)P², 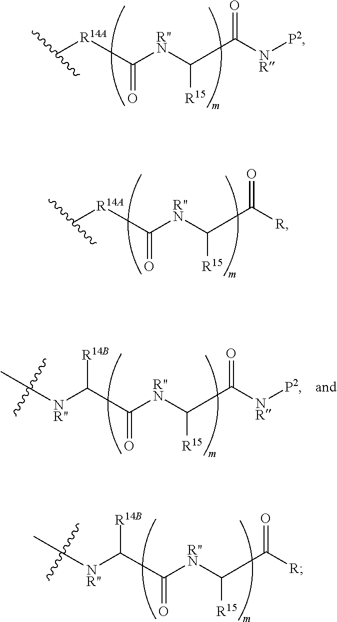
P¹ is selected from R″, —SO₂R″, 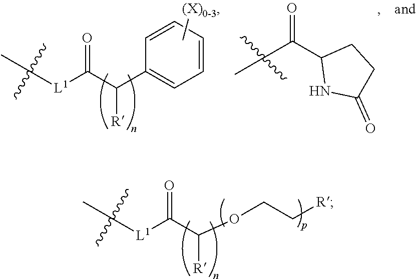
L¹ is absent or selected from 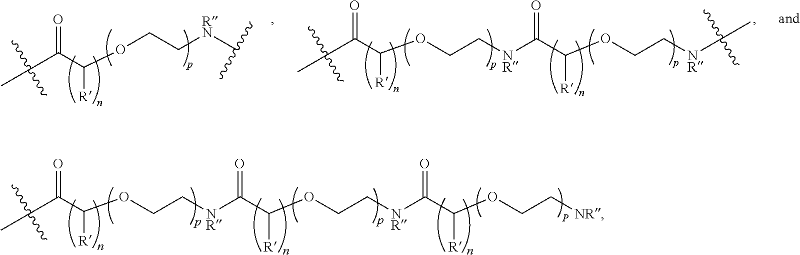
wherein the amino group of L¹ connects to the carbonyl group of P¹ to form an amide bond;
P² is selected from R″, 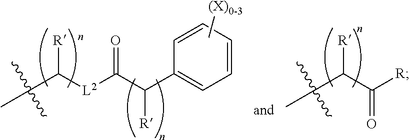
L² is absent or is selected from 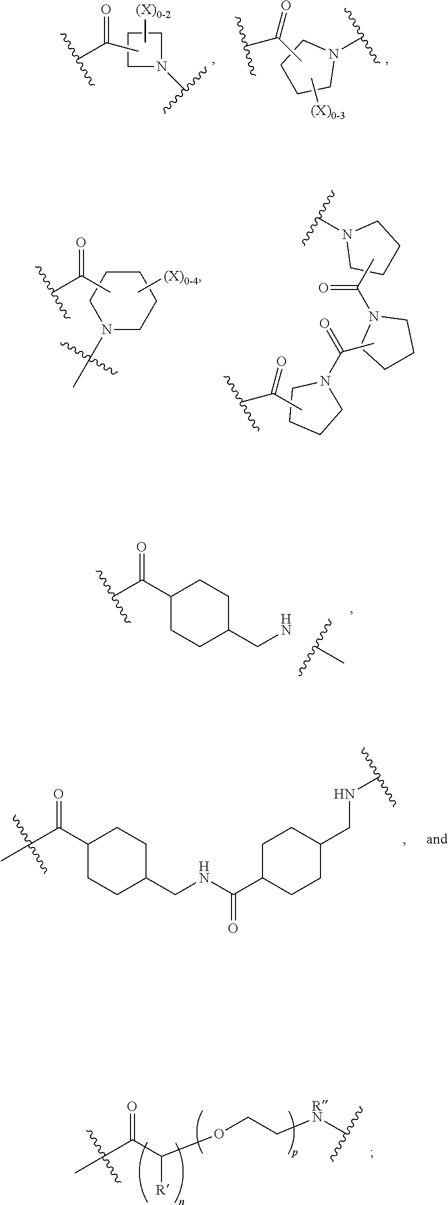
wherein the amino group of L² connects to the carbonyl group of P^{2′} to form an amide bond;
R¹ is an amino acid side chain of a natural amino acid or an amino acid side chain of an unnatural amino acid;
R² is an amino acid side chain of a natural amino acid or an amino acid side chain of an unnatural amino acid;
B¹ is C₁-6 alkylene;
C¹ is C₁-6 alkylene;
A¹ is selected from: 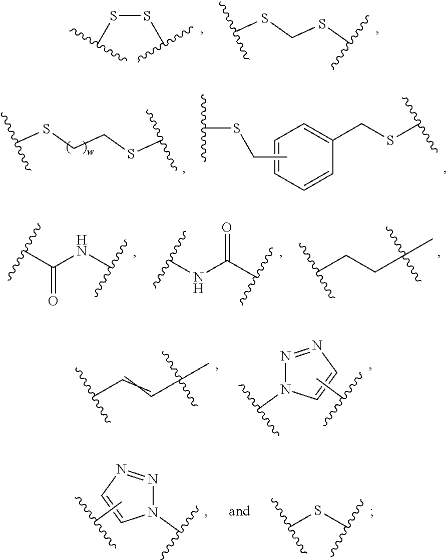
w is 1, 2, or 3;
R⁴ is selected from an amino acid side chain of a natural amino acid or an amino acid side chain of an unnatural amino acid;
R⁵ is selected from:
(i) an amino acid side chain of a natural amino acid,
(ii) an amino acid side chain of an unnatural amino acid,
(iii) L³-L³-Chelator, 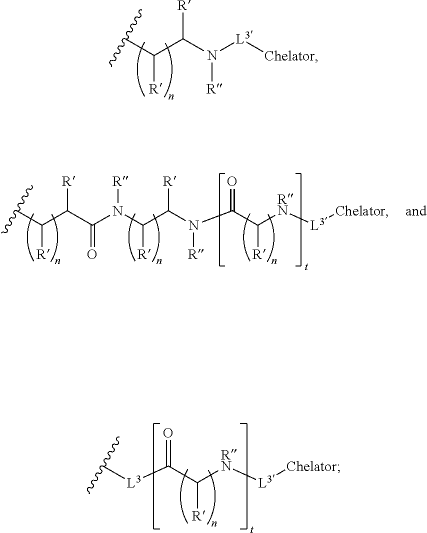
R⁶ is selected from:
(i) an amino acid side chain of a natural amino acid,
(ii) an amino acid side chain of an unnatural amino acid,
(iii) L³-L³-Chelator, 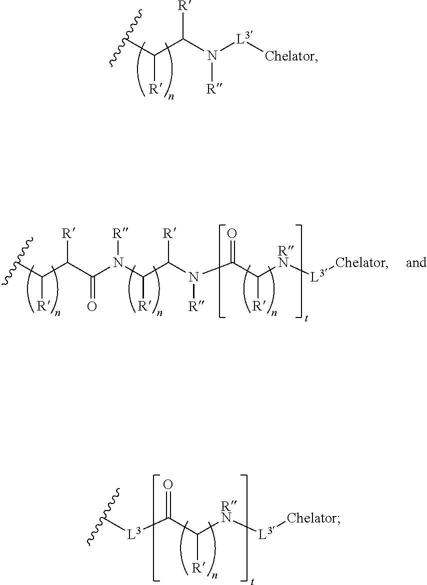
R⁷ is selected from:
(i) an amino acid side chain of a natural amino acid,
(ii) an amino acid side chain of an unnatural amino acid,
(iii) L³-L³-Chelator, 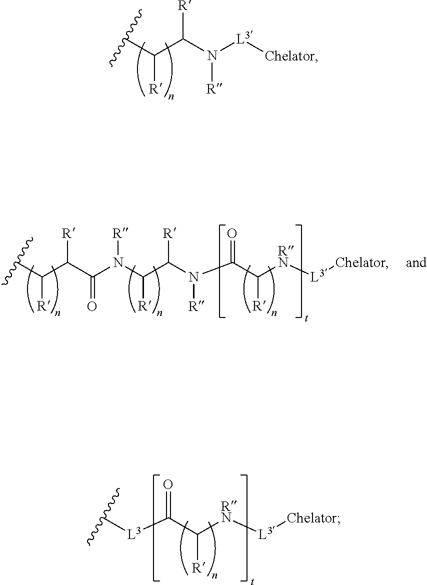
R⁸ is selected from an amino acid side chain of a natural amino acid or an amino acid side chain of an unnatural amino acid;
R⁹ is selected from:
(i) an amino acid side chain of a natural amino acid,
(ii) an amino acid side chain of an unnatural amino acid,
(iii) L³-L³-Chelator, 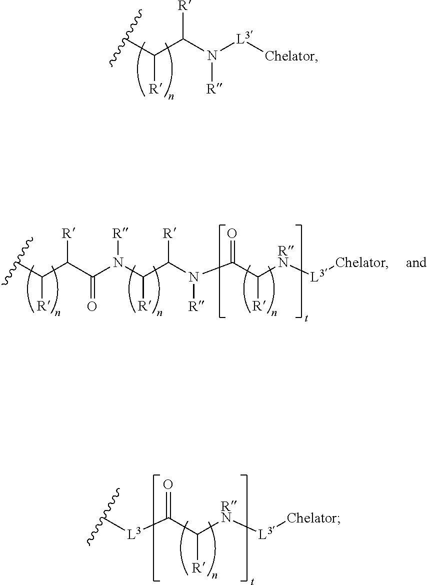
R¹⁰ is selected from:
(iii) an amino acid side chain of a natural amino acid,
(ii) an amino acid side chain of an unnatural amino acid,
(iii) L³-L^{3′}-Chelator, 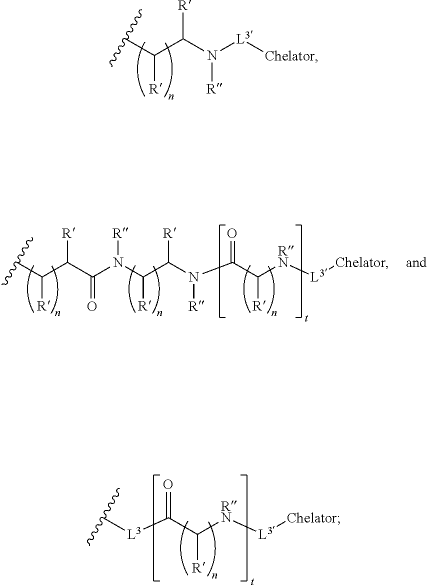
R¹² is selected from an amino acid side chain of a natural amino acid or an amino acid side chain of an unnatural amino acid;
R¹³ is selected from an amino acid side chain of a natural amino acid or an amino acid side chain of an unnatural amino acid;
R^{14A} is selected from: 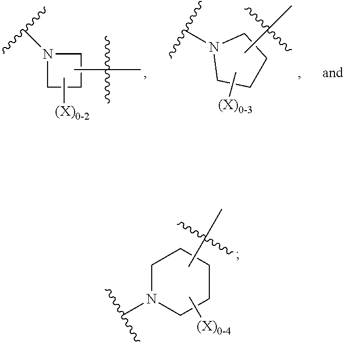
R^{14B} is selected from an amino acid side chain of a natural amino acid or an amino acid side chain of an unnatural amino acid;
R¹⁵ is an amino acid side chain of a natural amino acid or an amino acid side chain of an unnatural amino acid;
L³ is absent or selected from 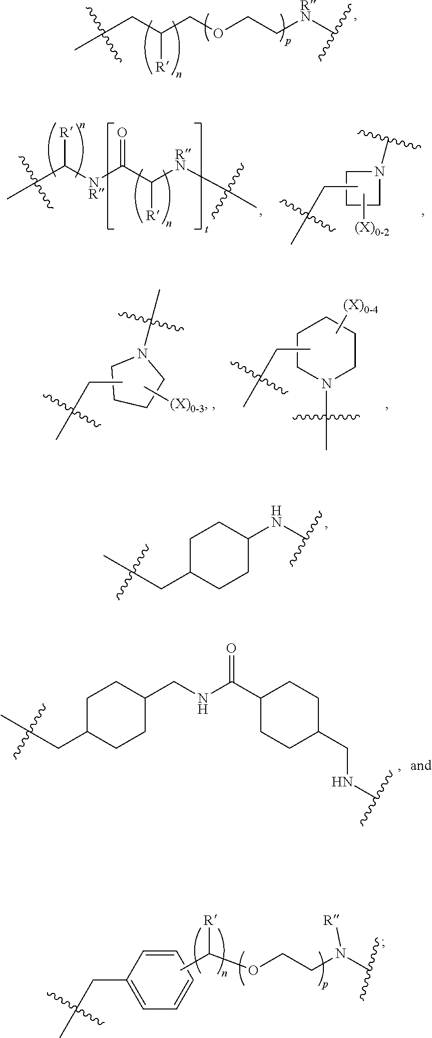
L^{3′} is absent or selected from 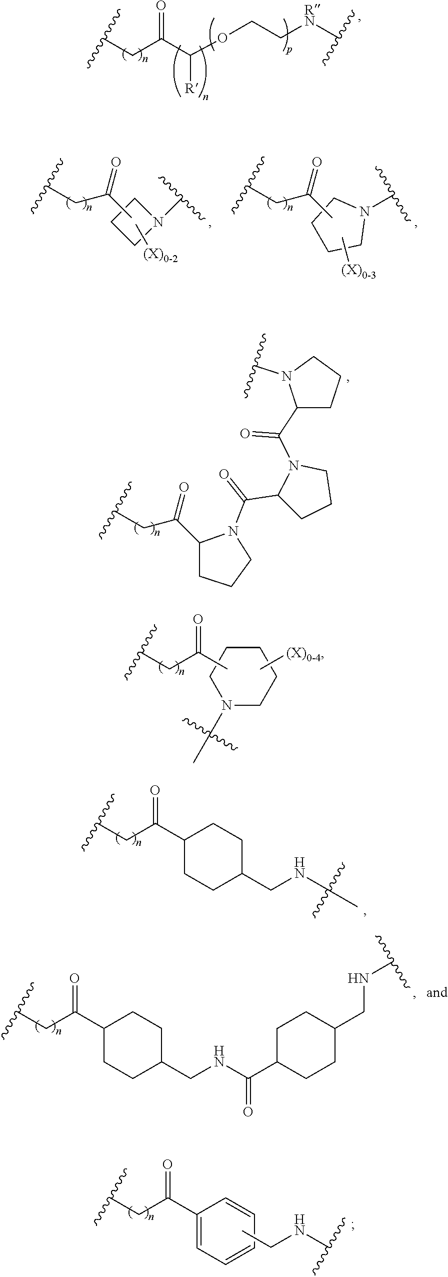
m is 0 or 1;
each n is independently an integer from 0 to 16;
each p is independently an integer from 0 to 30;
each t is independently 0, 1, 2, 3, 4, 5, or 6;
X is independently, for each occurrence, selected from halo, OH, C₁-C₆ alkyl, C₁-C₆ haloalkyl, N(R″)₂, —O—(C₁-C₆ alkyl);
R is independently, for each occurrence, selected from H, C₁-C₂₀ alkyl, C₁-C₆ haloalkyl, OH, —O—(C₁-C₆ alkyl), (C₁-C₆ alkyl)-OH, and N(R″)₂;
R′ is independently, for each occurrence, selected from H, OH, C₁-C₆ alkyl, C₁-C₆ haloalkyl, C₁-C₈ alkylamine, C₁-C₈ alkyl-OH, C(O)H, C(O)(C₁-C₆ alkyl), C(O)OH, C(O)O(C₁-C₆ alkyl), (C₁-C₆ alkyl)C(O)OH, (C₁-C₆ alkyl)C(O)O(C₁-C₆ alkyl), (C₁-C₆ alkyl)CO(C₁-C₆ alkyl), (C₁-C₆ alkyl)CS(C₁-C₆ alkyl), C(O)N(R″)₂, N(R″)₂, and N(R″)₃⁺; and
R″ is independently, for each occurrence, selected from H, C₁-C₆ alkyl, C₁-C₆ haloalkyl, (C₁-C₆ alkyl)C(O)OH, (C₁-C₆ alkyl)C(O)N(alkyl)₂, and C(O)O(C₁-C₆ alkyl);
wherein the cyclic peptide comprises one Chelator;
when a variable group R¹, R², R⁴, R⁵, R⁶, R⁷, R⁸, R⁹, R¹⁰, R¹², R¹³, or R¹⁵, is the amino acid side chain of a cyclic amino acid, the corresponding amino acid nitrogen of the peptide backbone of forms part of the cyclic group;
each alpha-carbon atom on the peptide backbone is optionally substituted with methyl;
and wherein the cyclic peptide optionally comprises a radionuclide.

2. The cyclic peptide of claim 1, wherein the cyclic peptide of Formula I is a cyclic peptide of Formula IIa or Formula IIb: 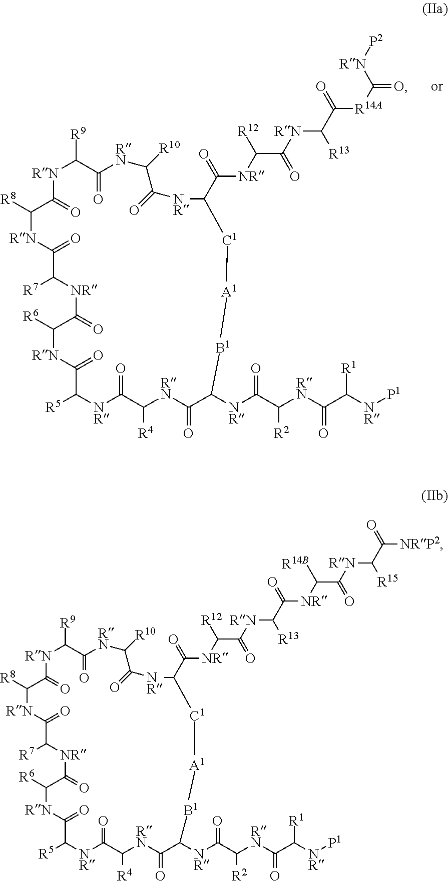
or a pharmaceutically acceptable salt thereof.

3. (canceled)

4. (canceled)

5. The cyclic peptide of claim 1, or a pharmaceutically acceptable salt thereof, wherein P¹ is selected from —SO₂R′, 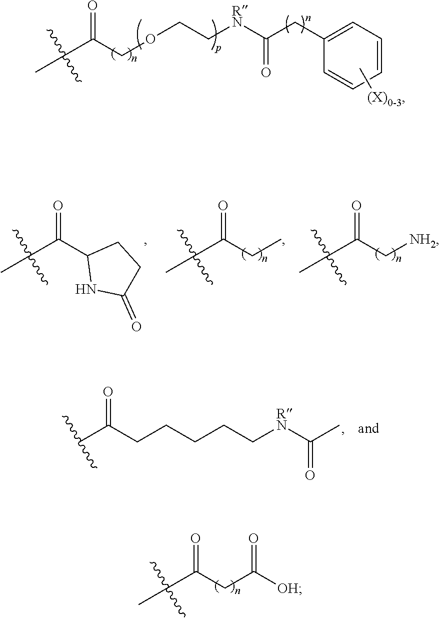
R′ is H, C₁-C₆ alkyl, C(O)(C₁-C₆ alkyl), C(O)OH, (C₁-C₆ alkyl)C(O)OH, and C(O)N(R″)₂;
R″ is independently, for each occurrence, H or C₁-C₆ alkyl;
n is an integer from 1 to 10; and
p is an integer from 1 to 12.

6. (canceled)

7. The cyclic peptide of claim 1, or a pharmaceutically acceptable salt thereof, wherein the C-terminus is selected from OH, N(R″)₂, 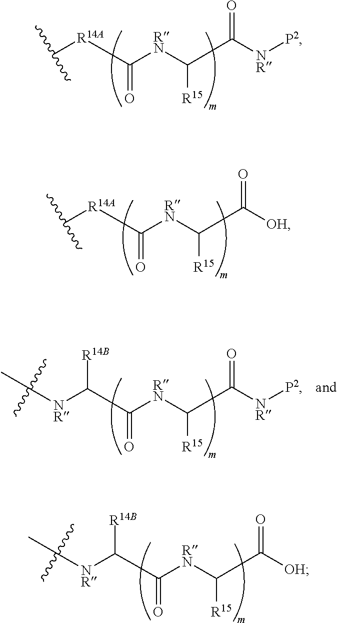
P² is R″ or P² is selected from 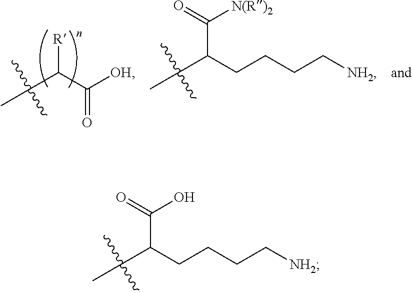
R′ is independently, for each occurrence, H or C₁-C₈ alkyl-OH;
R″ is independently, for each occurrence, H or C₁-C₆ alkyl;
p is an integer from 1 to 12; and
n is an integer from 1 to 4.

8. The cyclic peptide of claim 1, or a pharmaceutically acceptable salt thereof, wherein L³ is absent or selected from 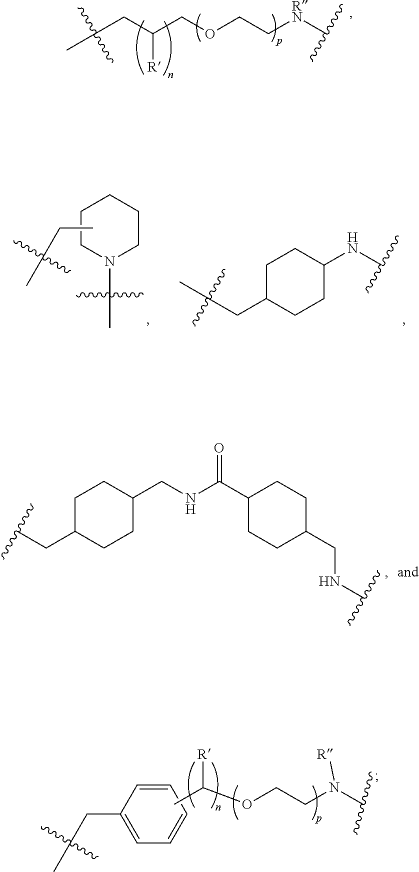
and
L^{3′} is absent or selected from

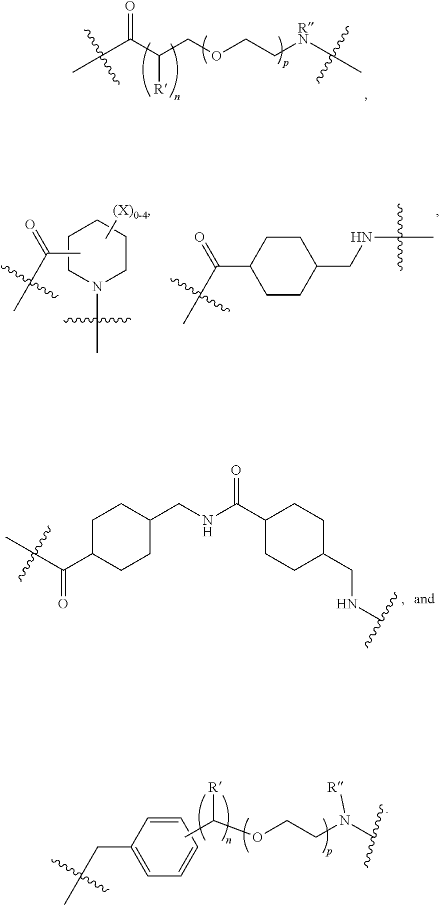

9. (canceled)

10. The cyclic peptide of claim 1, or a pharmaceutically acceptable salt thereof, wherein one of R⁵, R⁶, R⁷, R⁹, and R¹⁰ is selected from 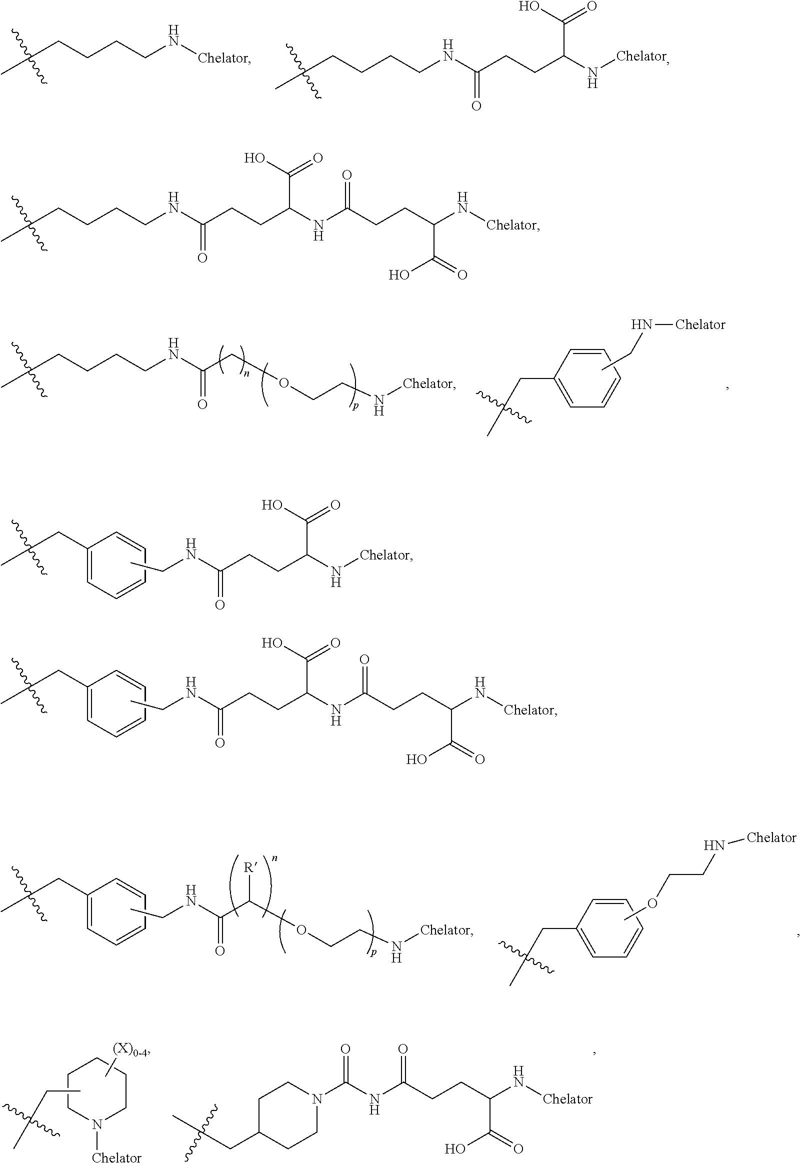 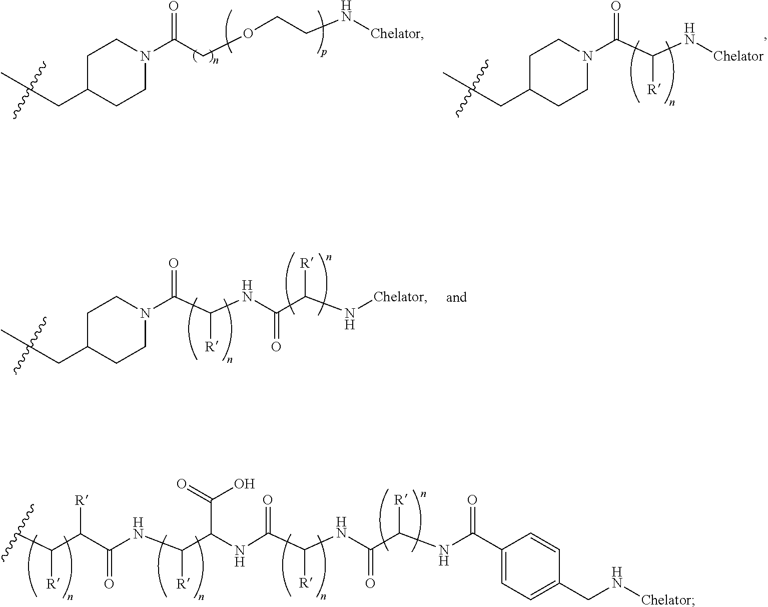
wherein R′ is selected from H, C(O)OH, C₁-C₆ alkyl, and (C₁-C₆ alkyl)S(C₁-C₆ alkyl);
n is 1 to 8; and
p is 0 to 12.

11. The cyclic peptide of claim 1, or a pharmaceutically acceptable salt thereof, wherein
R¹ is selected from the group consisting of an amino acid side chain of Ala, Cha, dAla, hCba, hCha, hLeu, HomoArg, HomoGlu, hTba, Leu, Met, and Nle;
R² is selected from the group consisting of an amino acid side chain of3CIY, 3FY, 4COOHPhe, 4FPhe, 4PyA, 7AzaW, Ala, alloThr, aMeNle, aMeTyr, Cba, Chg, dAla, FSY, HomoArg, HomoGlu, hTyr, hyVal, Nle, NMeNle, OMeTyr, Tba, ThpA, ThpG, Thr, and Tyr;
R⁴ is selected from the group consisting of an amino acid side chain of Abu, allolle, aMeVal, alloThr, betaMelle, ChG, Cpg, dAla, HomoGlu, HomoVal, Ile, Nle, NMeNle, NMeVal, Nva, PipAla, Tbg, tBuAla, Thr, and Val;
R⁵ is:
(i) selected from the group consisting of an amino acid side chain ofAla, dAla, Gly, HomoGlu, N(CH₂)₂COOHGly, NEtGly, Ser, and Sar; or
(ii) selected from the group consisting of: 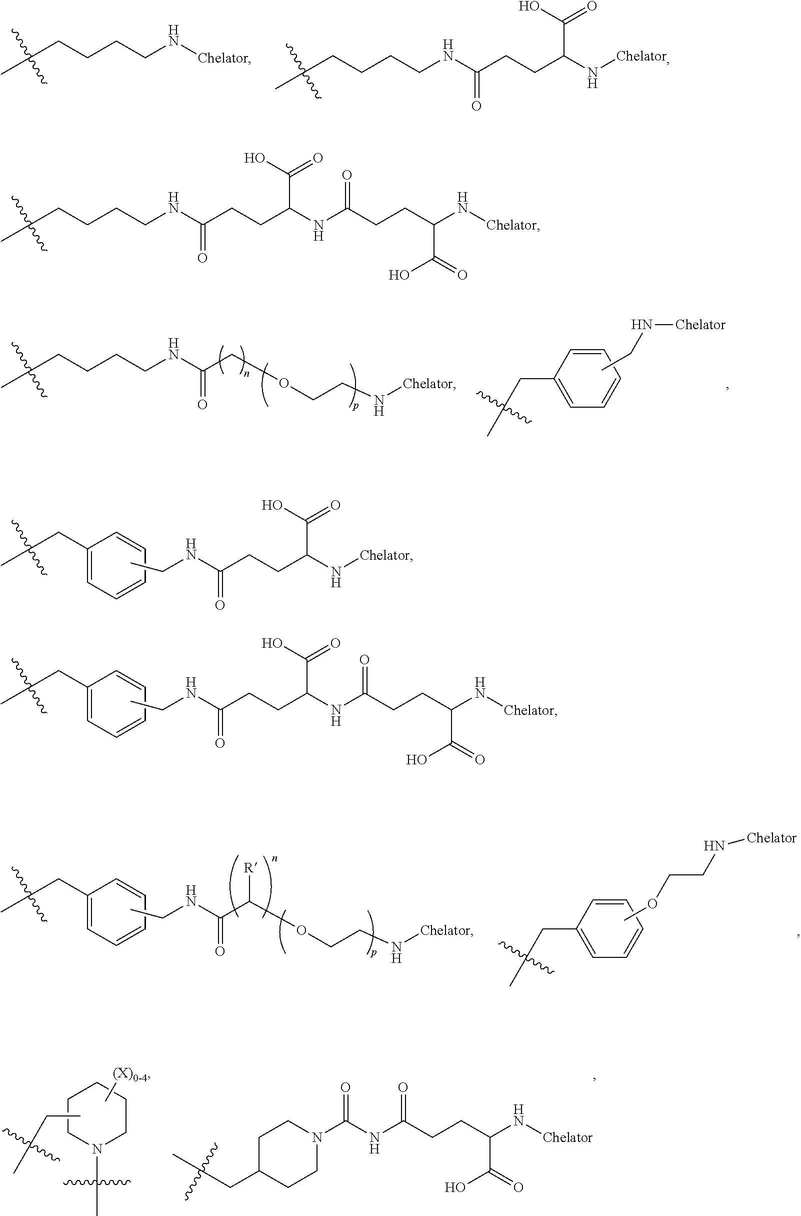 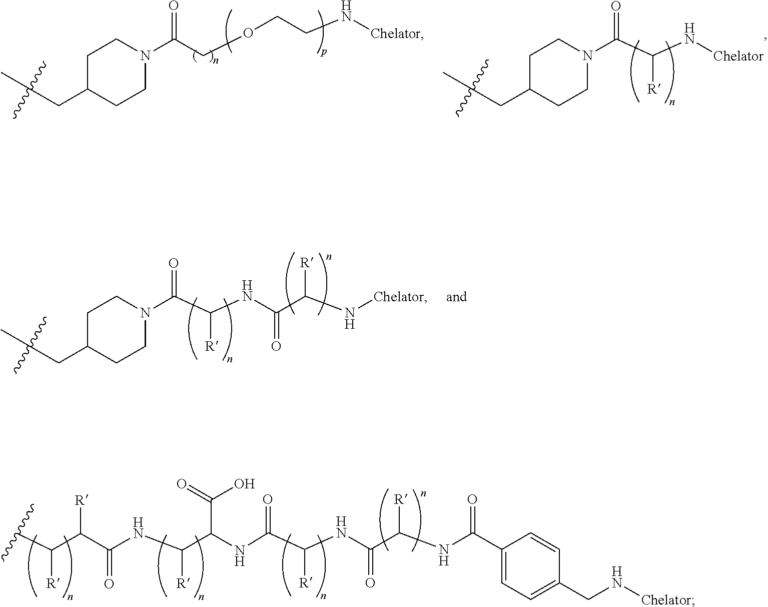
R⁶ is:
(i) selected from the group consisting of an amino acid side chain of Ala, aMeLys, Arg, Aze, Cit, Dab, dAla, Dap, dPro, HomoGlu, hSer, Lys, Lys(Ac), Lys(Me)2, Lys(Me)3, Nle, NMeLys, Orn, Pip, Pip(Ac)Ala, Pip(C4diacid)Ala, Pip(CH2COOH)Ala, Pip(CONH2)Ala, Pip(Me)Ala, PipAla, Pro, ThpA, and 4PyA; or
(ii) selected from the group consisting of: 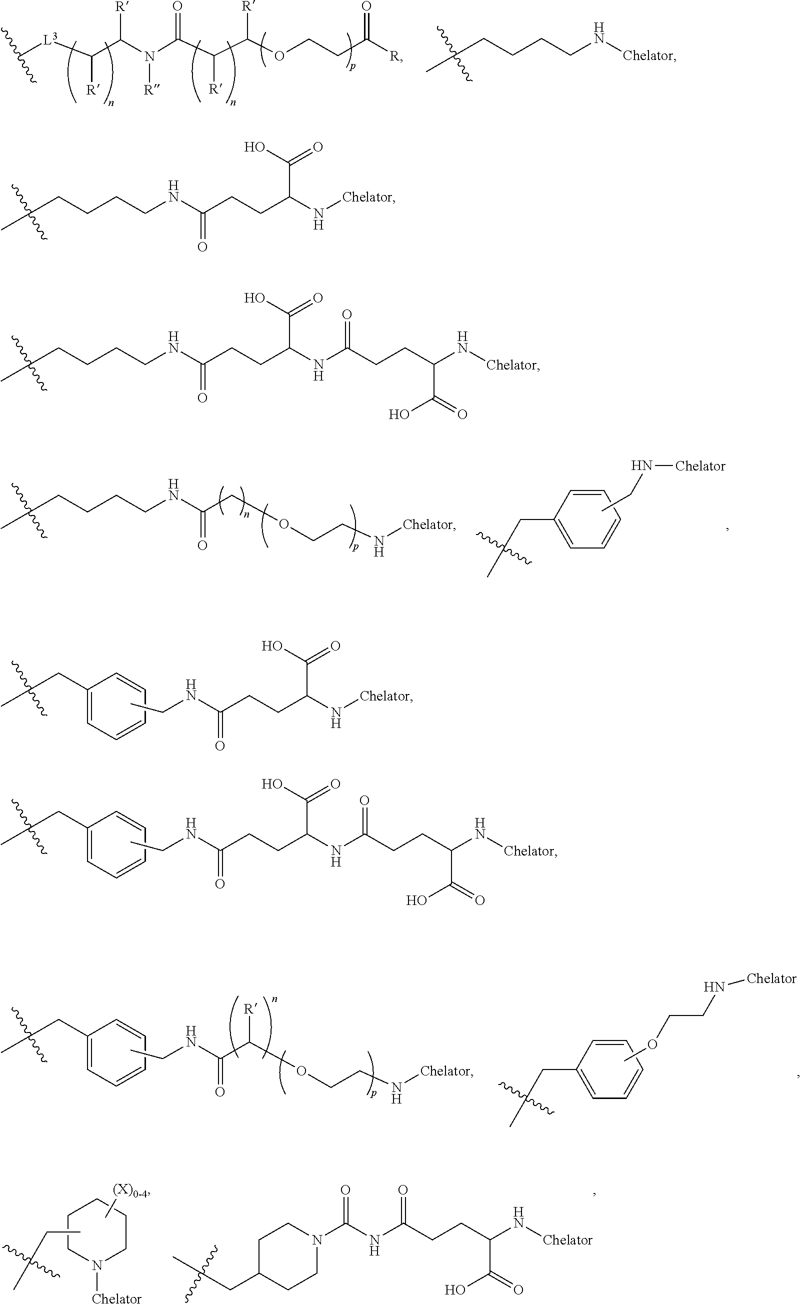 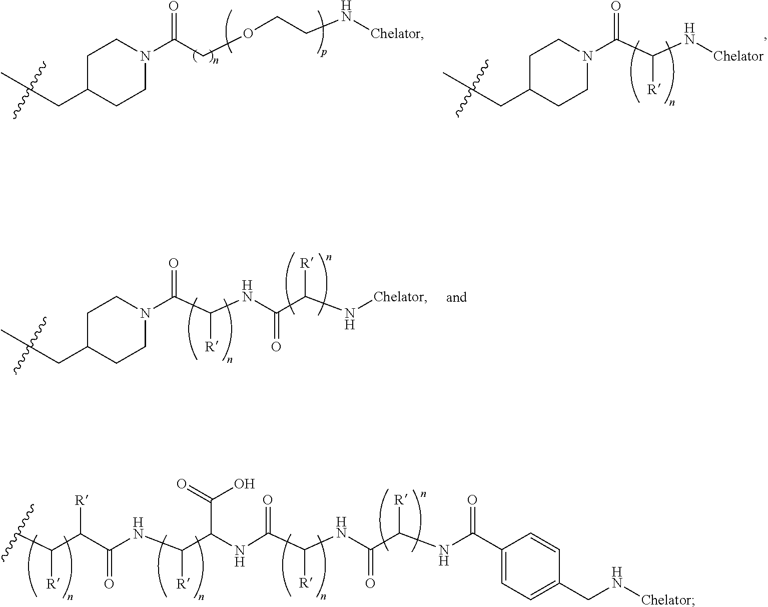
R⁷ is:
(i) selected from the group consisting of an amino acid side chain of (N-Me-ThpA), 4COOHPhe, 4OHCha, 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, and Tyr;
(ii) selected from the group consisting of: 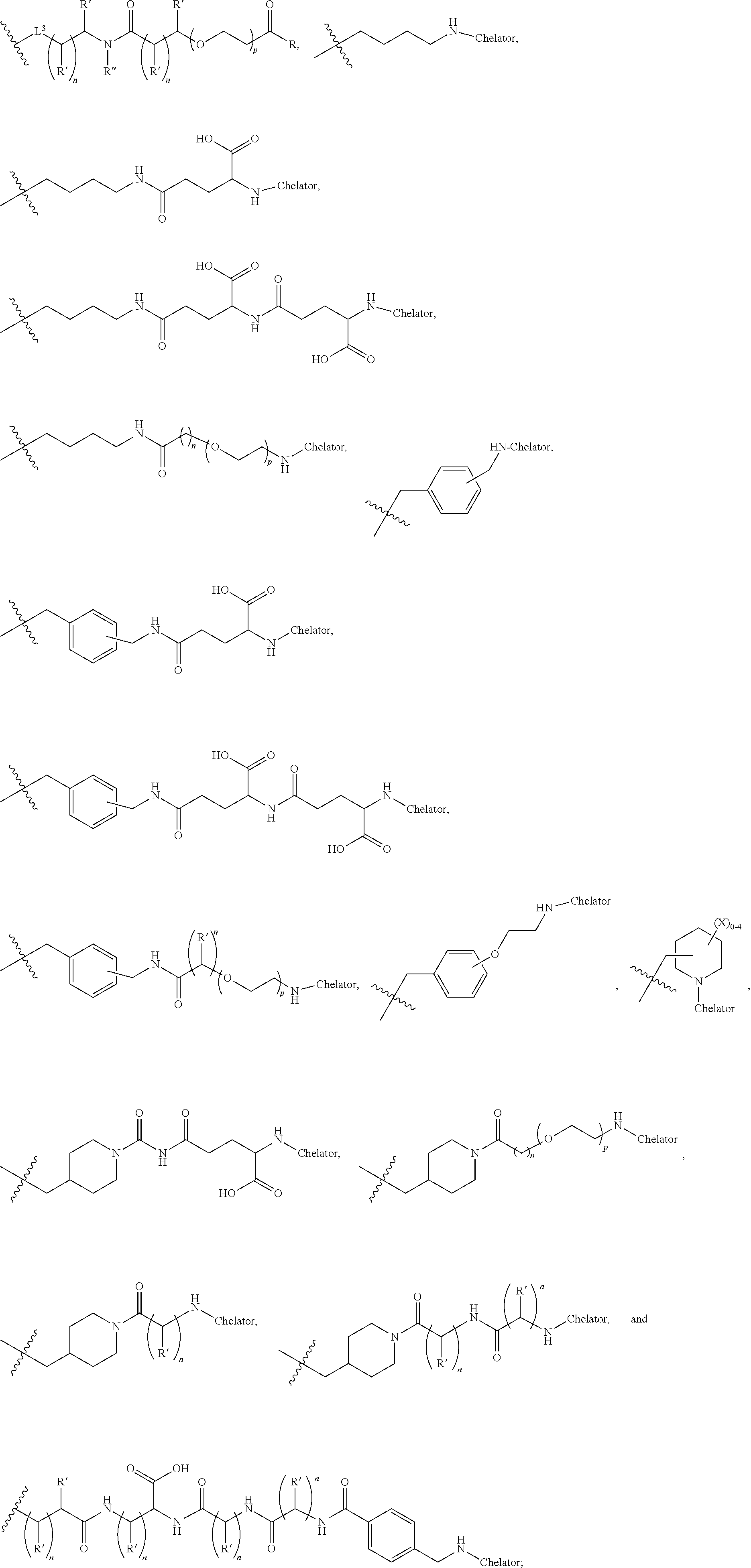
R⁸ is the group consisting of an amino acid side chain of Ala, CysAcid, Asp, Dab, dAla, FSY, HomoGlu, Asn, and Ser;
R⁹ is selected from the group consisting of an amino acid side chain of Abu, Aib, Ala, allolle, alloThr, aMeSer, aMeVal, ChG, Cpg, dAla, HomoGlu, hSer, hydroVal, Nle, NMeVal, Nva, Ser, Tbg, Thr, Val, 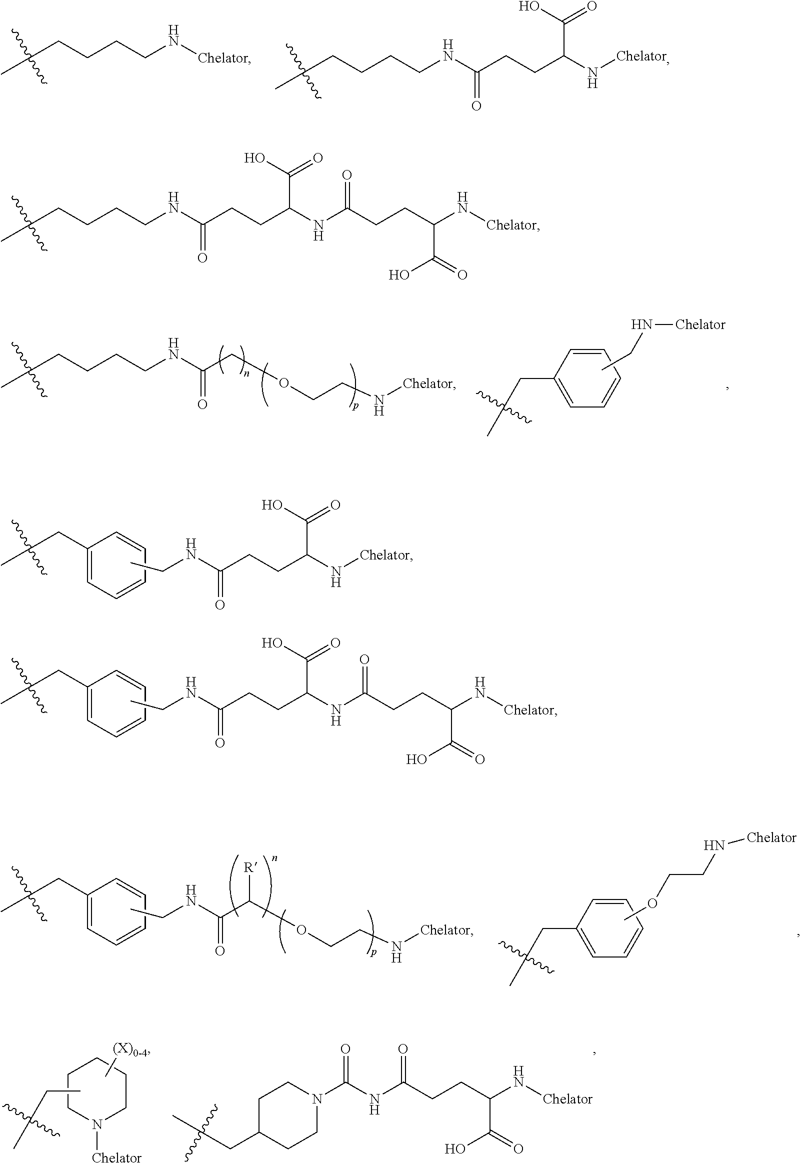 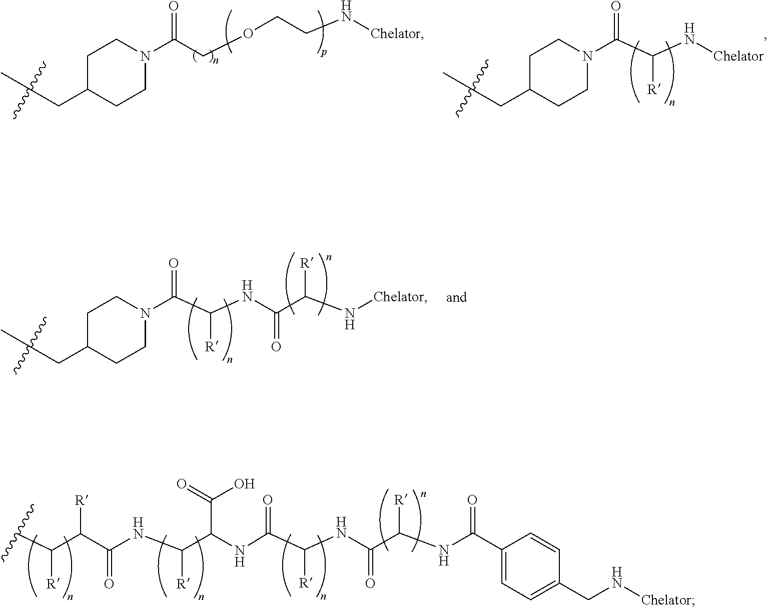
R¹⁰ is selected from the group consisting of an amino acid side chain of (4-carbamoyl-Phe), 2OHPhe, 3FY, 3OHPhe, 3PyA, 4FPhe, Ala, alloThr, aMeLeu, aMeTyr, Aph(Cbm), Cha, Chg, CyanoButric, dAla, FSY, HomoArg, HomoGlu, hyVal, Nle, NmeTyr, OmeTyr, Tba, ThpA, Thr, Tyr, 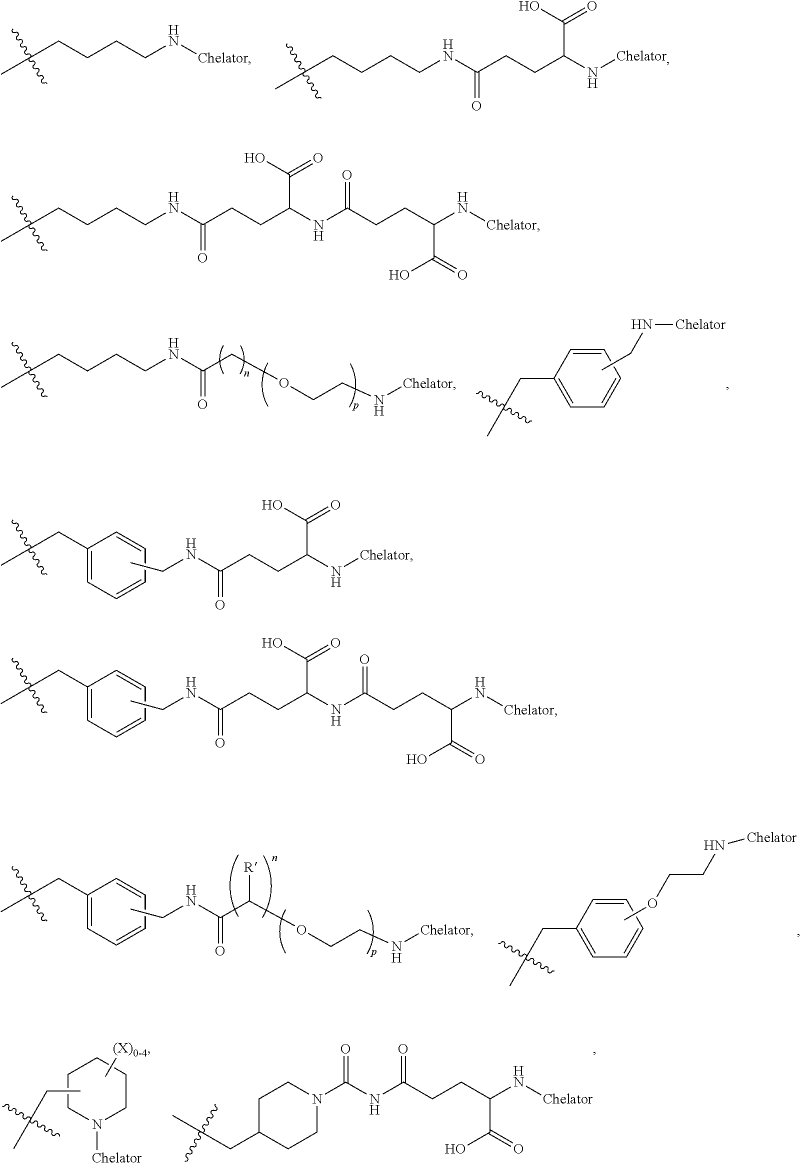 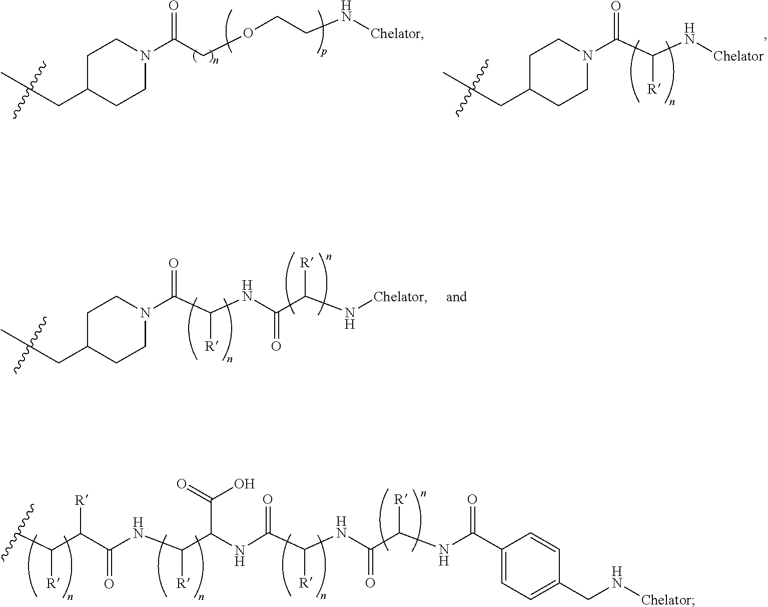
R¹² is selected from the group consisting of an amino acid side chain of (2S,3S-betaMe-5FW), 1Nal, 2MeW, 2Nal, 4CIW, 4FW, 4MeOW, 4MeW,5CIW, 5FW, 5MeOW, 5MeW, 7NW, Ala, aMeW, dAla, hF, HomoArg, HomoGlu, Trp(CH₂COOH), Trp(Me), and Trp; and
R¹³ is selected from the group consisting of an amino acid side chain of [(2S, 3R)beta-Me-Phe], [(2S, 3R)beta-OH-Phe], [(2S, 3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, ChA, dAla, Phe, His, HomoArg, HomoGlu, HomoPhe, Leu, Nle, NMeNle, NMePhe, Tbg, tBuAla; and
R^{14A} is

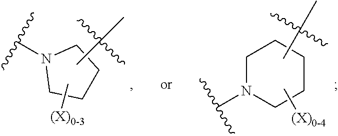

12. (canceled)

13. The cyclic peptide of claim 1, or a pharmaceutically acceptable salt thereof, wherein
R¹ is selected from the group consisting of an amino acid side chain of Ala, Cha, dAla, hCba, hCha, hLeu, HomoArg, HomoGlu, hTba, Leu, Met, and Nle;
R² is selected from the group consisting of an amino acid side chain of 3CIY, 3FY, 4COOHPhe, 4FPhe, 4PyA, 7AzaW, Ala, alloThr, aMeNle, aMeTyr, Cba, Chg, dAla, FSY, HomoArg, HomoGlu, hTyr, hyVal, Nle, NMeNle, OMeTyr, Tba, ThpA, ThpG, Thr, and Tyr;
R⁴ is selected from the group consisting of an amino acid side chain of Abu, allolle, aMeVal, alloThr, betaMelle, ChG, Cpg, dAla, HomoGlu, hyVal, Ile, Nle, NMeNle, NMeVal, Nva, PipAla, Tbg, tBuAla, Thr, and Val;
R⁵ is:
(i) selected from the group consisting of an amino acid side chain of Ala, dAla, Gly, HomoGlu, N(CH₂)₂COOHGly, NEtGly, Ser, and Sar; or
(ii) selected from the group consisting of: 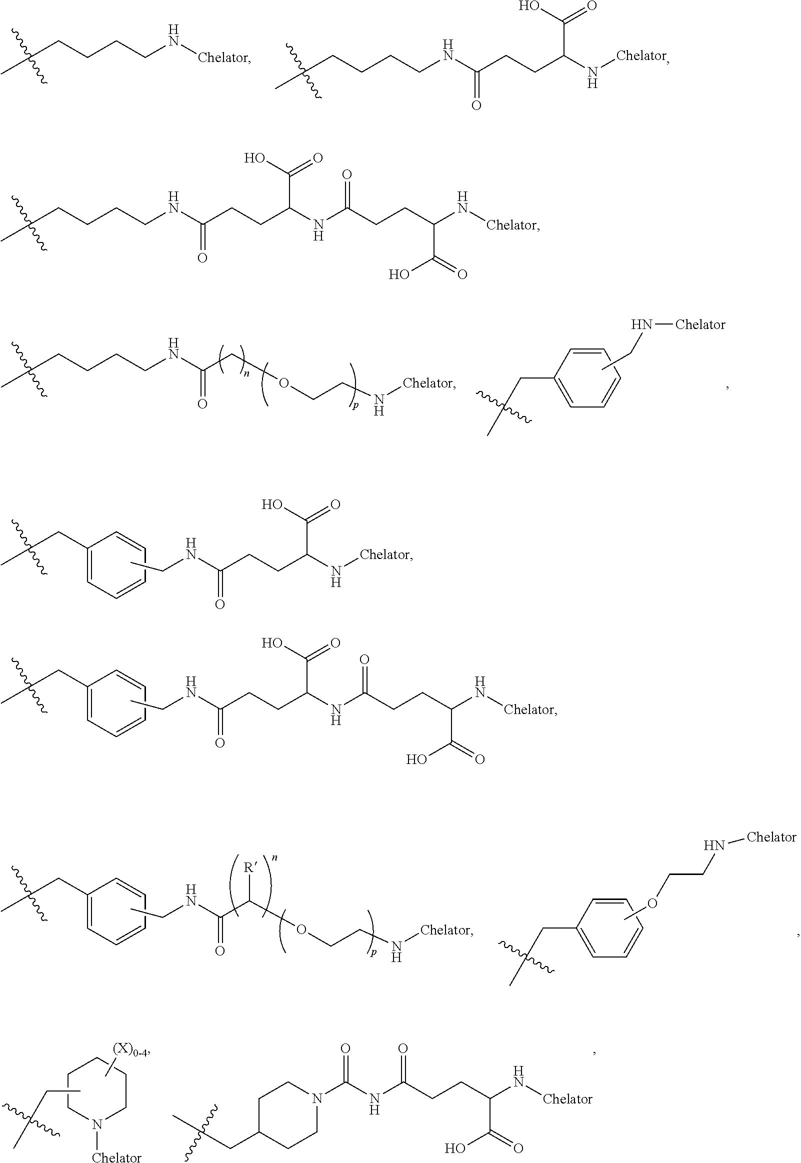 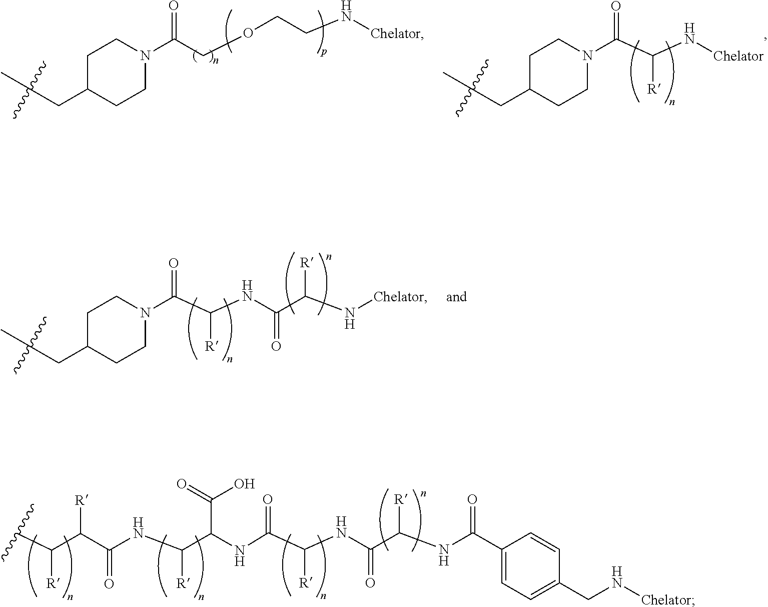
R⁶ is:
(i) selected from the group consisting of an amino acid side chain of Ala, aMeLys, Arg, Aze, Cit, Dab, dAla, Dap, dPro, HomoGlu, hSer, Lys, Lys(Ac), Lys(Me)2, Lys(Me)3, Nle, NMeLys, Orn, Pip, Pip(Ac)Ala, Pip(C4diacid)Ala, Pip(CH2COOH)Ala, Pip(CONH2)Ala, Pip(Me)Ala, PipAla, Pro, ThpA, and 4PyA;
(ii) selected from the group consisting of: 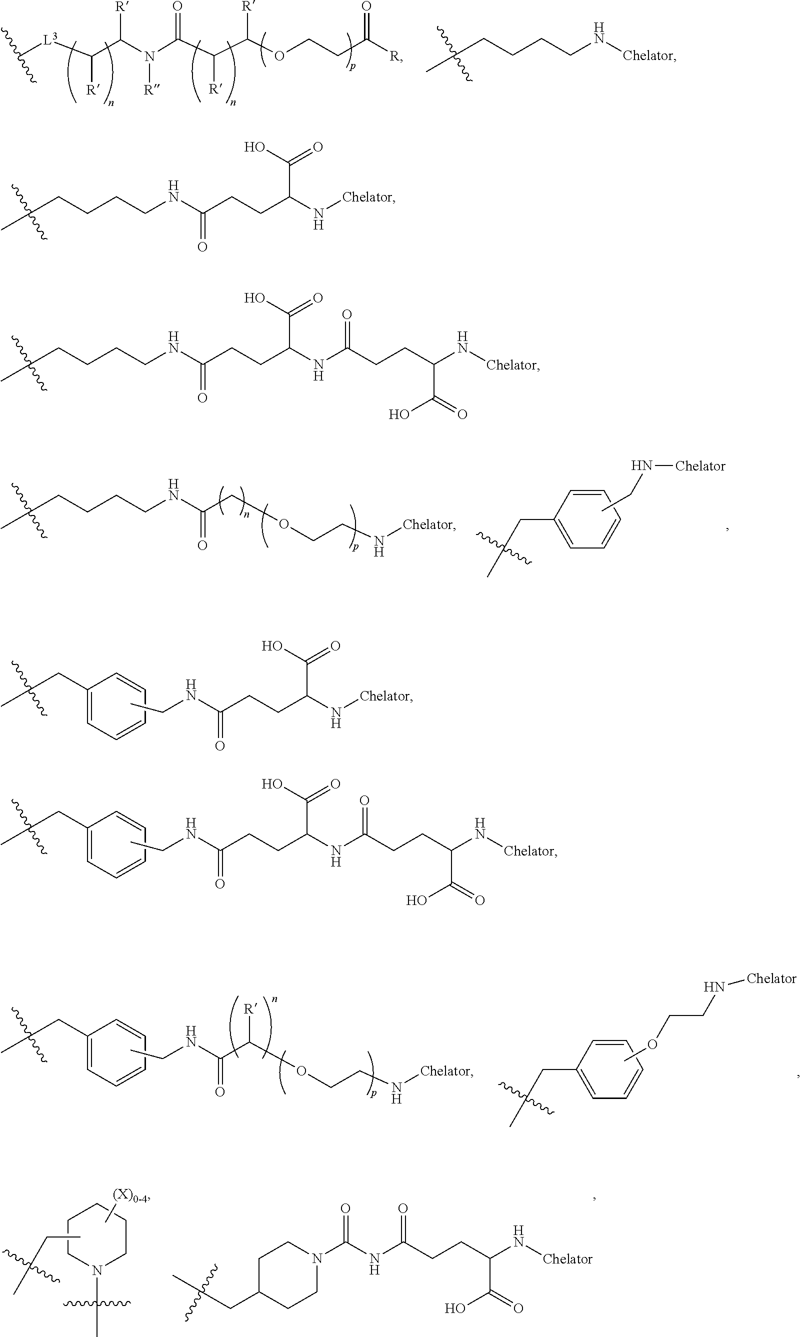 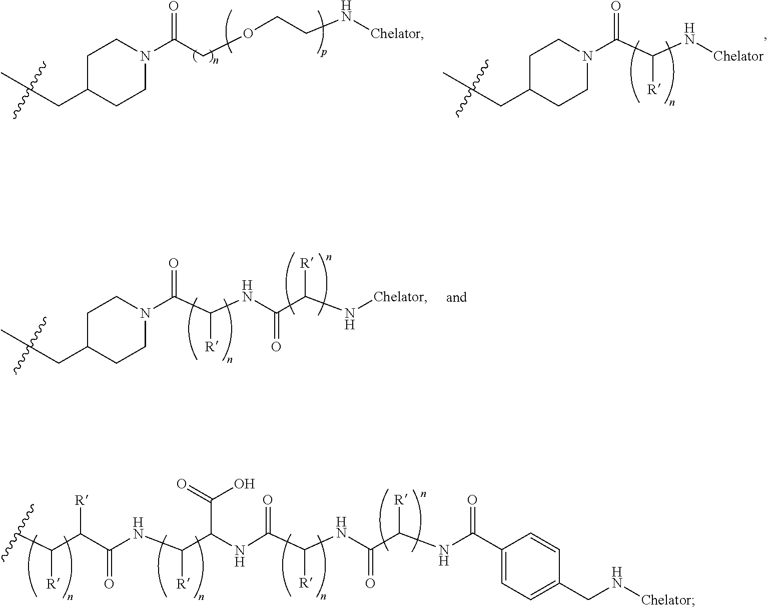
R⁷ is:
(i) selected from the group consisting of an amino acid side chain of (N-Me-ThpA), 4COOHPhe, 4OHCha, 4PyA, Ala, alloThr, aMeLeu, CHA, dAla, HomoGlu, hSer, hTyr, Pro, Pip(C4diacid)Ala, Pip(CH2COOH)Ala, PipAla, Thr, ThpA, andTyr;
(ii) selected from the group consisting of: 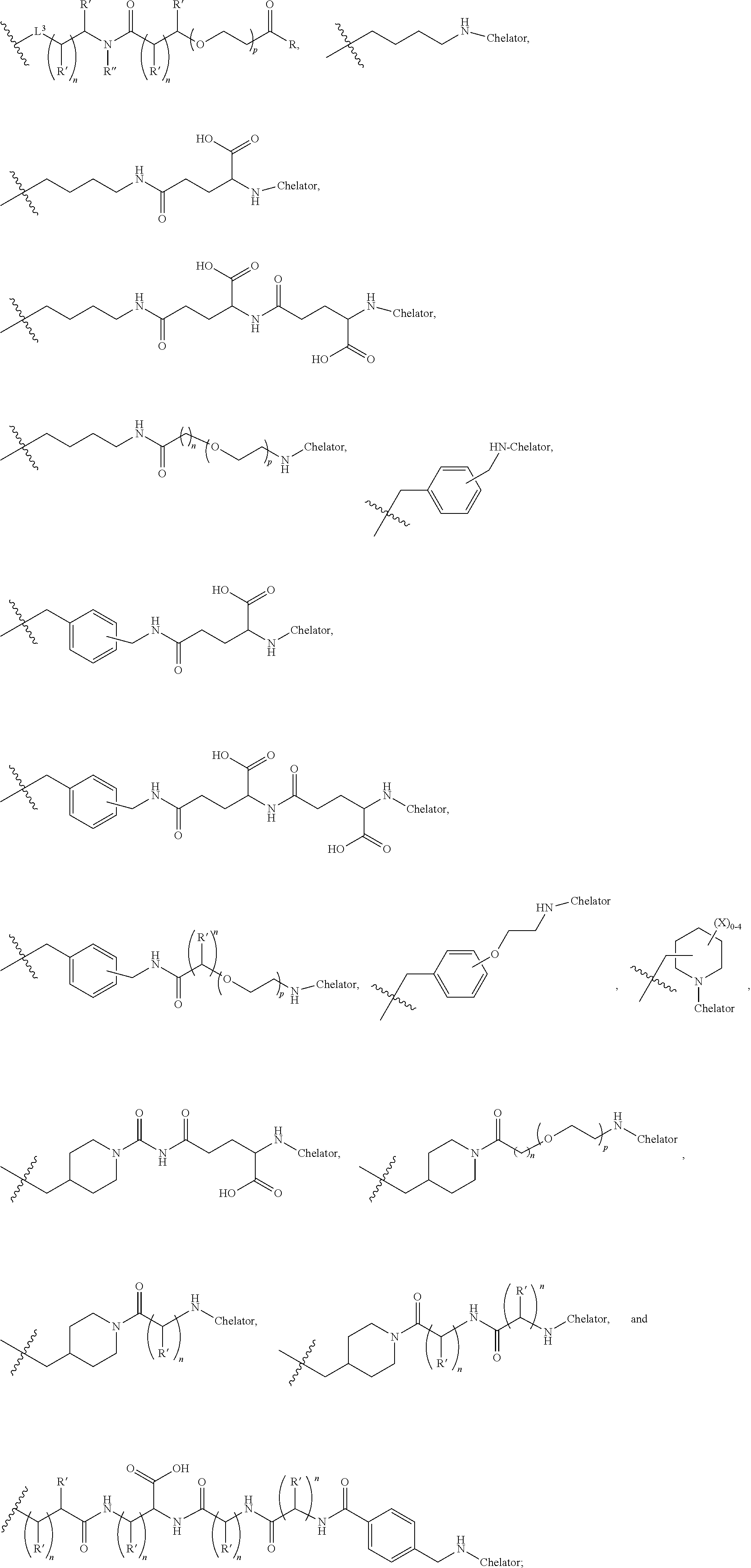
R⁸ is the group consisting of an amino acid side chain of Ala, CysAcid, Asp, Dab, dAla, FSY, HomoGlu, Asn, and Ser;
R⁹ is the group consisting of an amino acid side chain of selected from Abu, Aib, Ala, allolle, alloThr, aMeSer, aMeVal, ChG, Cpg, dAla, HomoGlu, hSer, hydroVal, Nle, NmeVal, Nva, Ser, Tbg, Thr, and Val; or 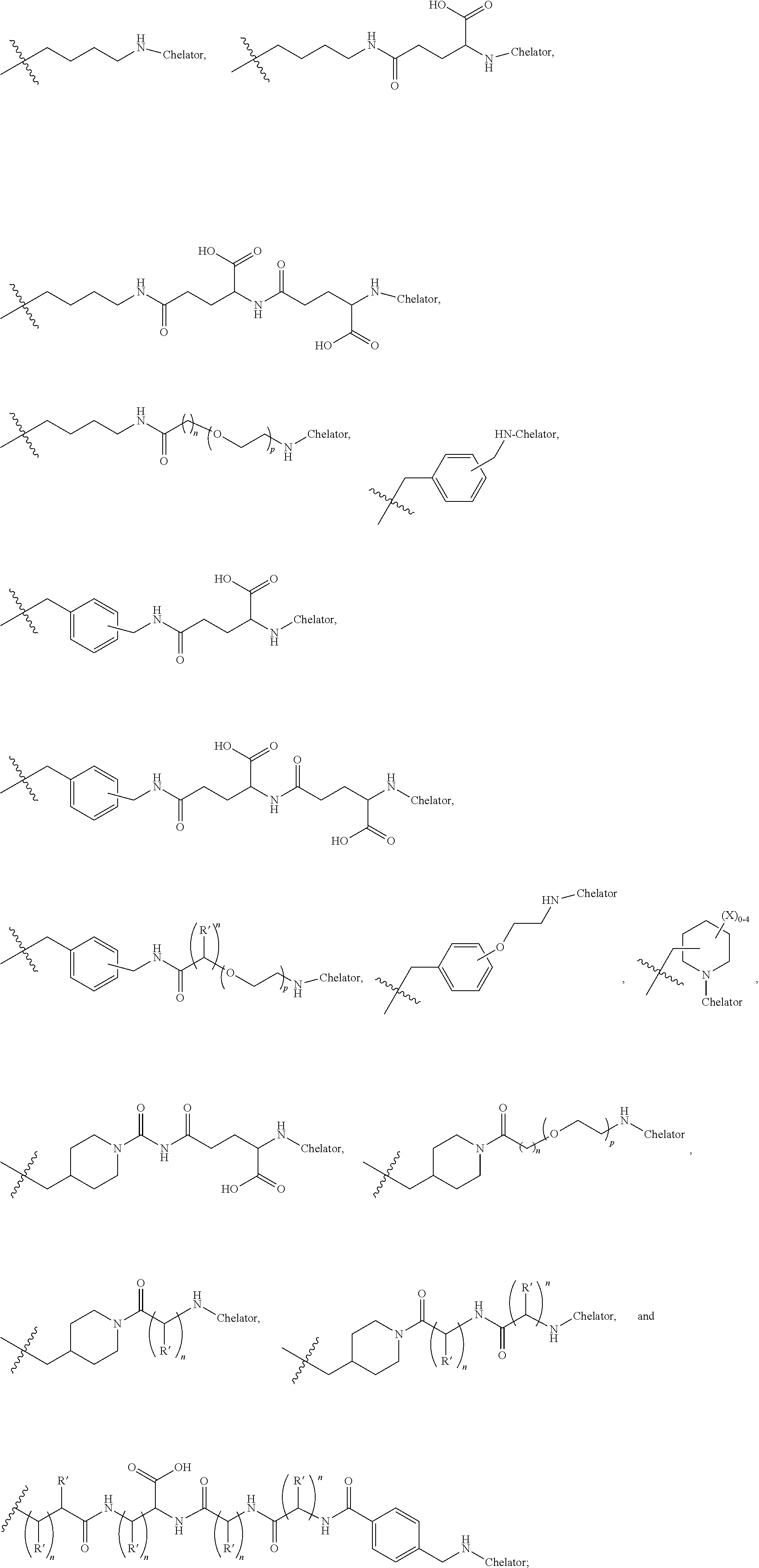
R¹⁰ is:
(i) selected from the group consisting of an amino acid side chain of (4-carbamoyl-Phe), 20HPhe, 3FY, 3OHPhe, 3PyA, 4FPhe, Ala, alloThr, aMeLeu, aMeTyr, Aph(Cbm), Cha, Chg, CyanoButric, dAla, FSY, HomoArg, HomoGlu, hyVal, Nle, NMeTyr, OMeTyr, Tba, ThpA, Thr, and Tyr; or
(ii) selected from the group consisting of: 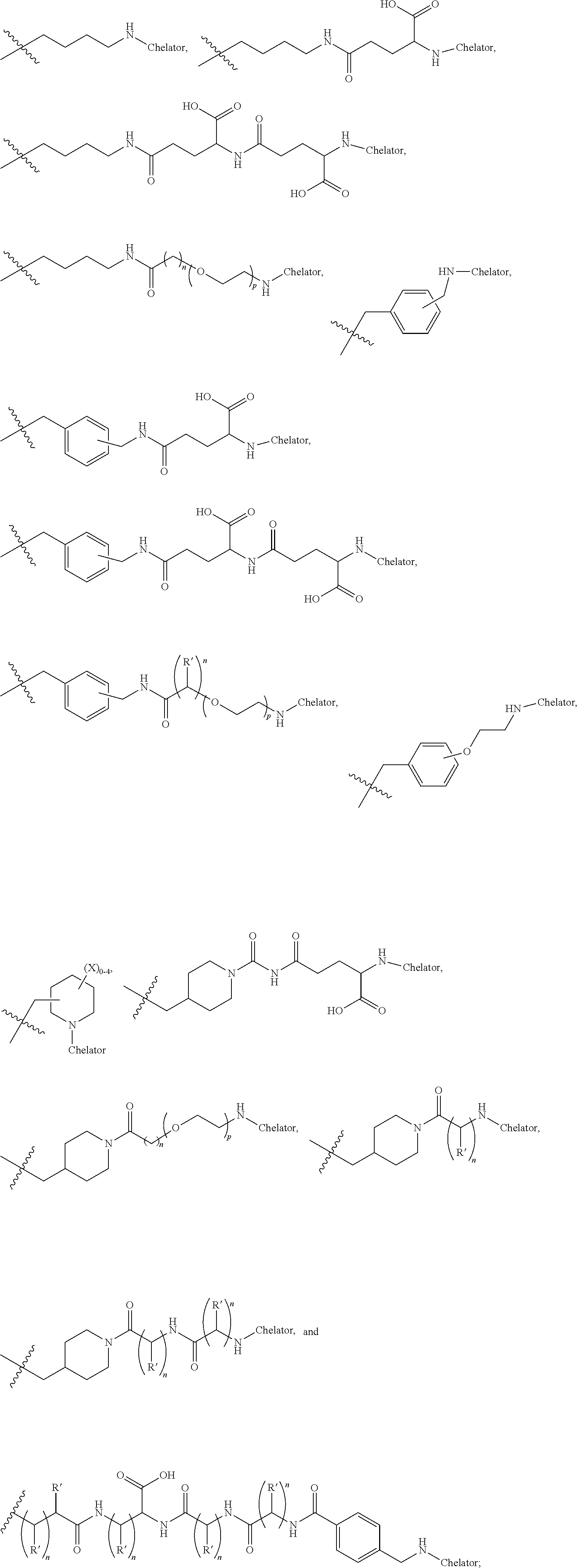
R¹² is selected from the group consisting of an amino acid side chain of (2S,3S-betaMe-5FW), 1Nal, 2MeW, 2Nal, 4CIW, 4FW, 4MeOW, 4MeW, 5CIW, 5FW, 5MeOW, 5MeW, 7NW, Ala, aMeW, dAla, hF, HomoArg, HomoGlu, Trp(CH₂COOH), Trp(Me), and Trp;
R¹³ is selected from the group consisting of an amino acid side chain of [(2S, 3R)beta-Me-Phe], [(2S, 3R)beta-OH-Phe], [(2S, 3S)beta-Me-Phe], 2FPhe, 2MePhe, 3aminomethylPhe, 3ClPhe, 3CNPhe, 3FPhe, 3OHPhe, 3PyA, 4aminomethylPhe, 4CNPhe, 4COOHPhe, 4FPhe, 4PyA, Ala, aMeNle, aMePhe, ChA, dAla, Phe, His, HomoArg, HomoGlu, HomoPhe, Leu, Nle, NMeNle, NMePhe, Tbg, and tBuAla;
R^{14B} is selected from the group consisting of an amino acid side chain of 4PipAla, alloThr, aMeSer, Dab, Dap, dGlu, dLys, dThr, Glu, gE, hydroxyVal, Lys, NMeSer, Orn, Ser, and Thr; and
R¹⁵ is selected from the group consisting of an amino acid side chain of dAla, dLys, dSer, dGlu, Gly, and betaAla.

14. The cyclic peptide of claim 1, wherein
R¹ is selected from the group consisting of an amino acid side chain of hLeu, hCha, hCba, and Nle;
R² is an amino acid side chain of Tyr;
R⁴ is selected from the group consisting of an amino acid side chain of Ile, Tbg, and Val;
R⁵ is an amino acid side chain of Gly or Sar;
R⁶ is:
(i) selected from the group consisting of an amino acid side chain of PipAla, Lys, ThpA, Pip(CH2COOH)Ala, Cit, Lys(Ac), Pip(Ac)Ala, Pip(CONH2)Ala, Pip(Me)Ala, Lys(Me)2, and Lys(Me)3; or
(ii) selected from the group consisting of: 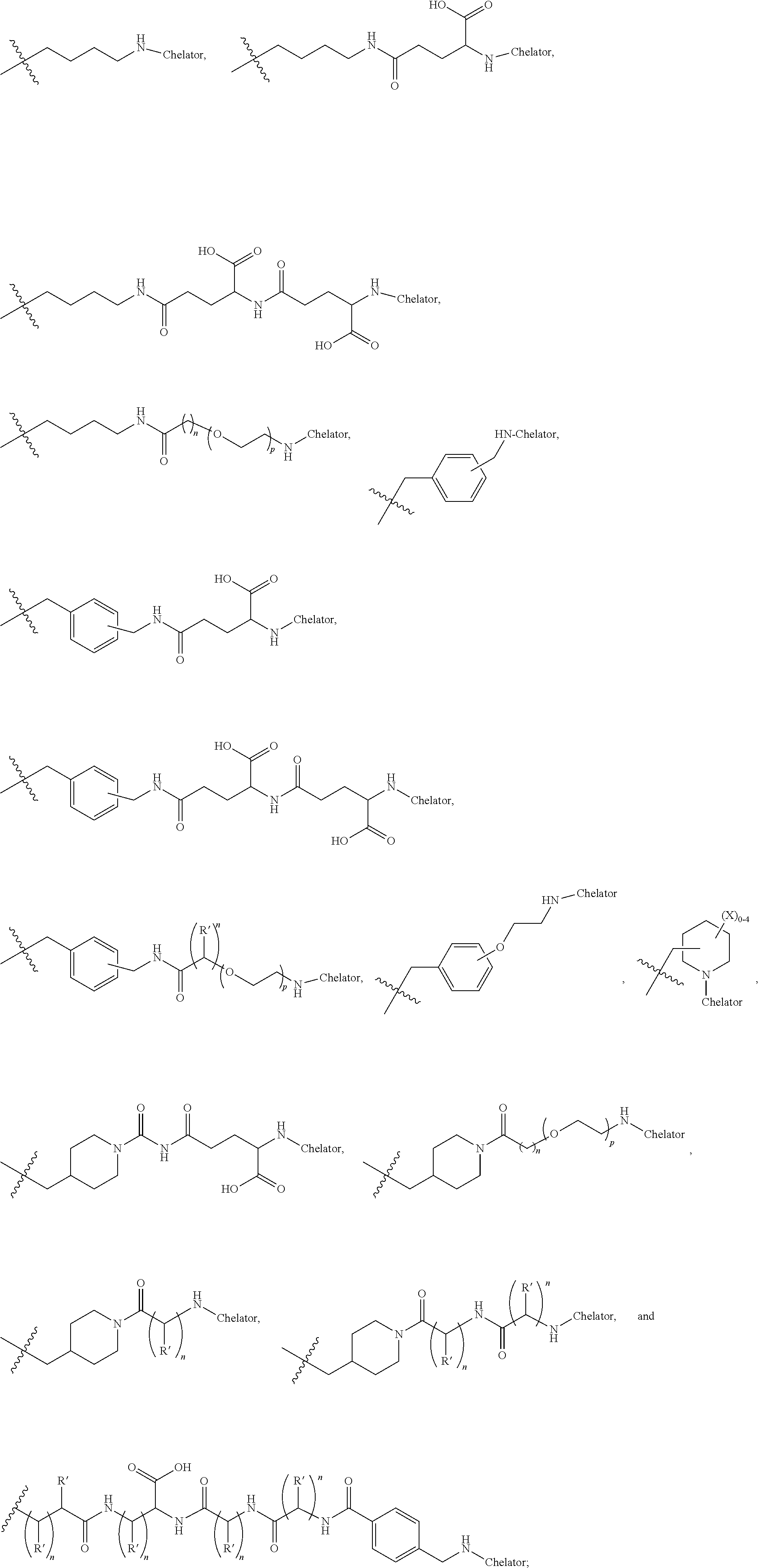
R⁷ is:
(i) selected from the group consisting of an amino acid side chain of 4COOHPhe, 4Pya, PipAla, and ThpA; or
(ii) selected from the group consisting of: 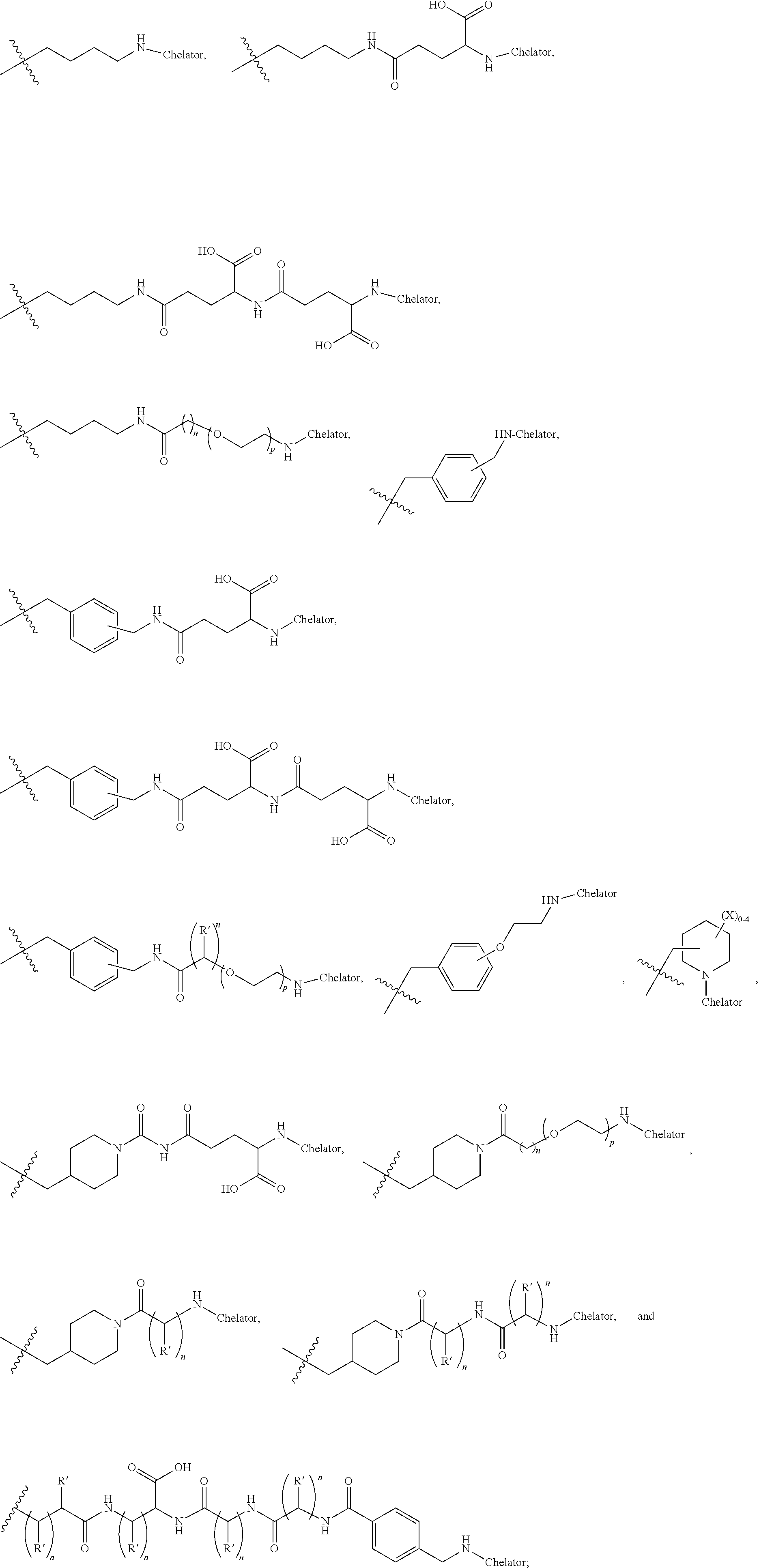
R⁸ is an amino acid side chain of Asp or CysAcid;
R⁹ is an amino acid side chain of Val;
R¹⁰ is an amino acid side chain of Tyr;
R¹² is an amino acid side chain of 5FW or 1Nal;
R¹³ is an amino acid side chain of Phe or [(2S, 3S)beta-Me-Phe];
R^{14B} is an amino acid side chain of hydroxyVal or Thr; and
R¹⁵ is selected from the group consisting of an amino acid side chain of dAla, dSer, dGlu, Gly, and betaAla.

15. The cyclic peptide of claim 1, or a pharmaceutically acceptable salt thereof, wherein
B¹ is CH₂ or C(CH₃)₂;
C¹ is CH₂ or C(CH₃)₂; and
A¹ is

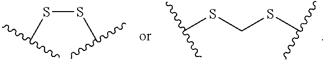

16. (canceled)

17. A cyclic peptide of Formula VI: 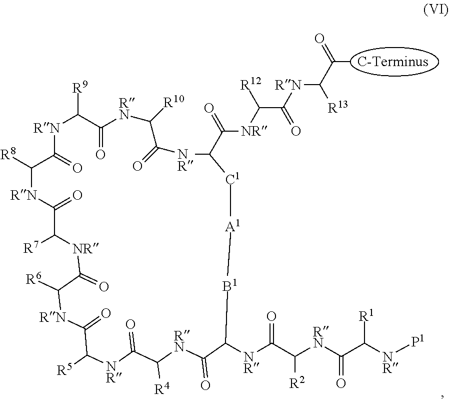
or a pharmaceutically acceptable salt thereof,
wherein:
the C-Terminus is selected from R, N(R″)P², 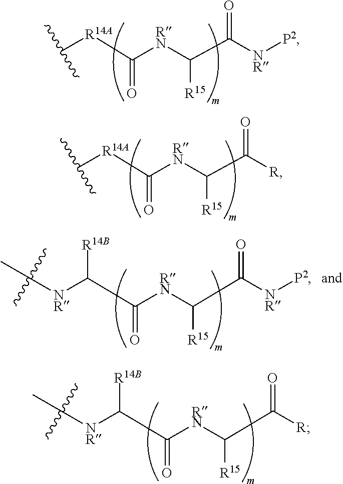
P¹ is selected from R″, —SO₂R″, -L¹-Chelator, 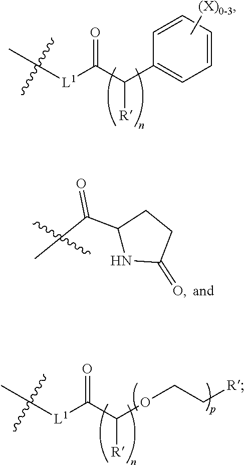
L¹ is absent or selected from 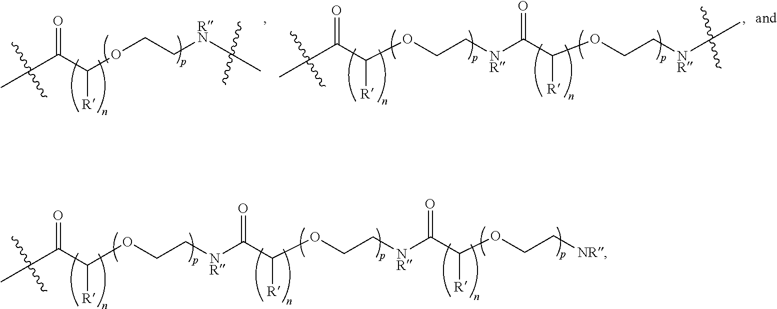
wherein the amino group of L¹ connects to the carbonyl group of P¹ or Chelator to form an amide bond;
P² is selected from -L²-Chelator, 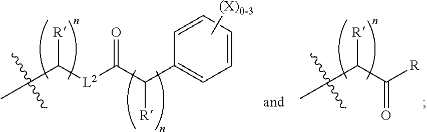
L² is absent or selected from 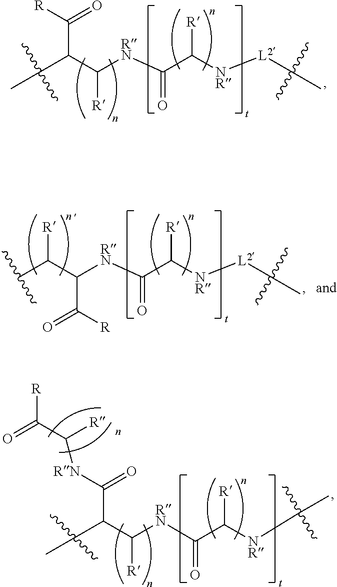
wherein the amino group of L² connects to the carbonyl group of L^{2′} or P² to form an amide bond;
L^{2′} is absent or is selected from: 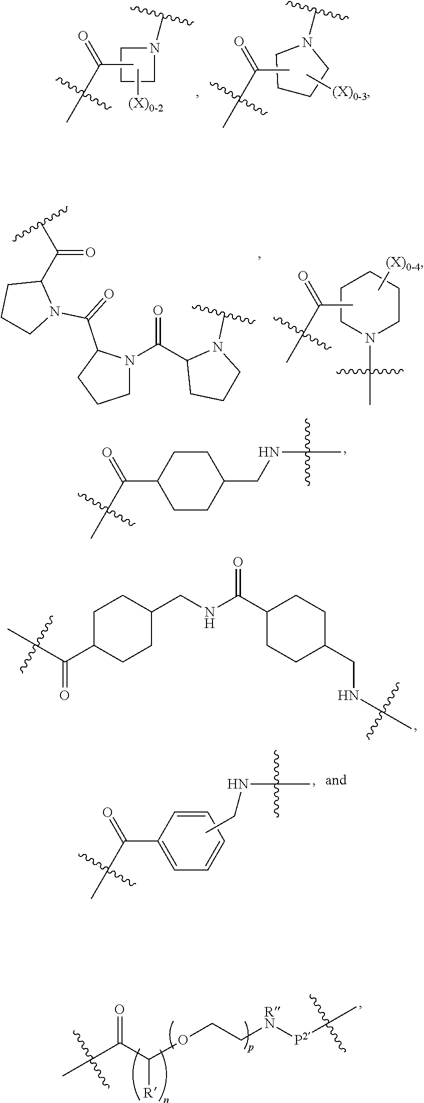
wherein the amino group of L^{2′} connects to the carbonyl group of P^{2′} or Chelator to form an amide bond;
P^{2′} is absent or is 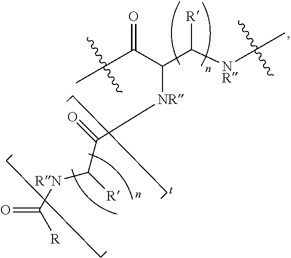
wherein the amino group of P^{2′} connects to the carbonyl group of Chelator to form an amide bond;
R¹ is an amino acid side chain of a natural amino acid or an amino acid side chain of an unnatural amino acid;
R² is selected from:
(i) an amino acid side chain of a natural amino acid,
(ii) an amino acid side chain of an unnatural amino acid,
(iii) L³-Chelator, 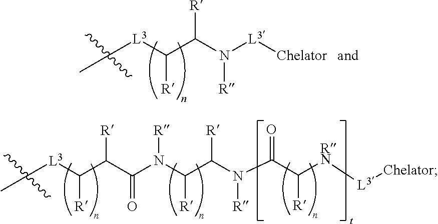
B¹ is C₁-6 alkylene;
C¹ is C₁-6 alkylene;
A¹ is selected from: 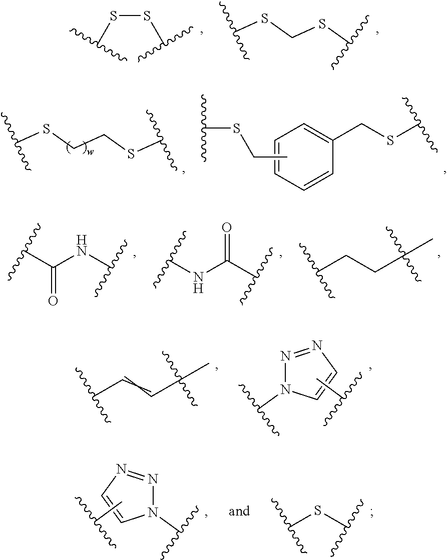
w is 1, 2, or 3;
R⁴ is selected from an amino acid side chain of a natural amino acid and an amino acid side chain of an unnatural amino acid;
R⁵ is selected from an amino acid side chain of a natural amino acid and an amino acid side chain of an unnatural amino acid;
R⁶ is selected from:
(i) an amino acid side chain of a natural amino acid,
(ii) an amino acid side chain of an unnatural amino acid, and
(iii) a structure selected from 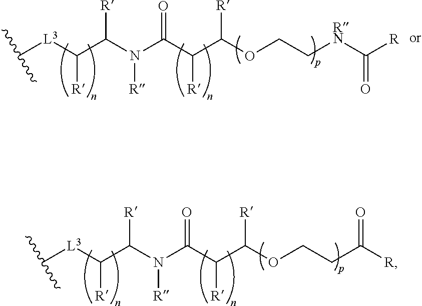
R⁷ is selected from:
(i) an amino acid side chain of a natural amino acid,
(ii) and an amino acid side chain of an unnatural amino acid, and
(iii) 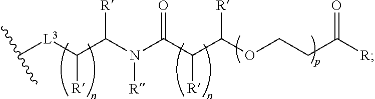
R⁸ is selected from an amino acid side chain of a natural amino acid and an amino acid side chain of an unnatural amino acid;
R⁹ is selected from an amino acid side chain of a natural amino acid and an amino acid side chain of an unnatural amino acid;
R¹⁰ is selected from an amino acid side chain of a natural amino acid and an amino acid side chain of an unnatural amino acid;
R¹² is selected from:
(i) an amino acid side chain of a natural amino acid,
(ii) an amino acid side chain of an unnatural amino acid,
(iii) L³-Chelator, 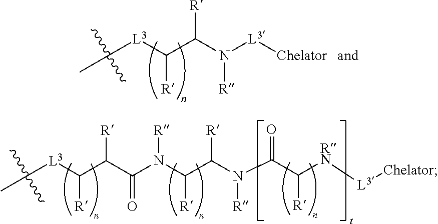
R¹³ is selected from:
(i) an amino acid side chain of a natural amino acid,
(ii) an amino acid side chain of an unnatural amino acid,
(iii) L³-Chelator, 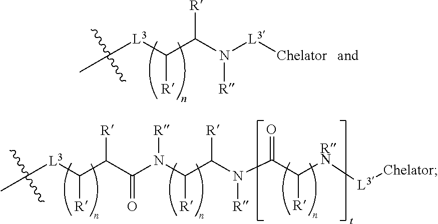
R^{14A} is selected from: 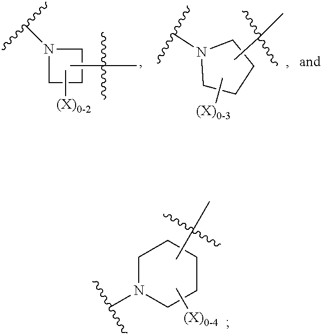
R^{14B} is selected from:
(i) an amino acid side chain of a natural amino acid,
(ii) an amino acid side chain of an unnatural amino acid,
R¹⁵ is an amino acid side chain of a natural amino acid or an amino acid side chain of an unnatural amino acid;
L³ is absent or selected from 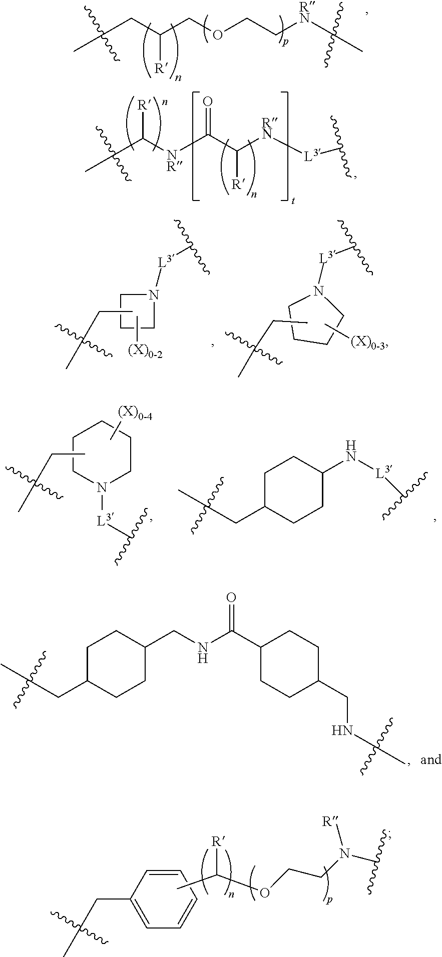
L^{3′} is absent or selected from 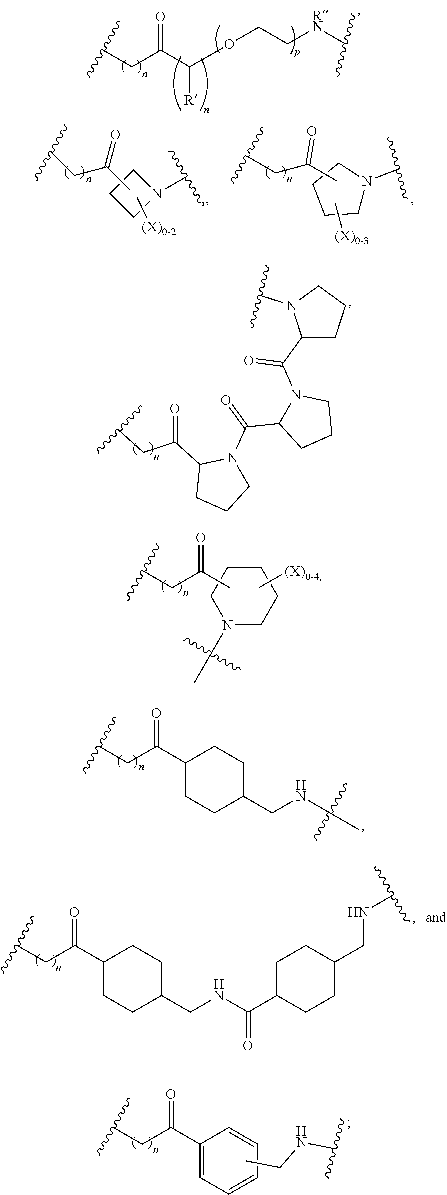
m is 0 or 1;
n′ is independently an integer from 1 to 16;
each n is independently an integer from 0 to 16;
each p is independently an integer from 0 to 30;
each t is independently 0, 1, 2, 3, 4, 5, or 6;
X is independently, for each occurrence, selected from halo, OH, C₁-C₆ alkyl, and C₁-C₆ haloalkyl;
R is independently, for each occurrence, selected from H, C₁-C₂₀ alkyl, C₁-C₆ haloalkyl, OH, —O—(C₁-C₆ alkyl), (C₁-C₆ alkyl)-OH, and N(R″)₂;
R′ is independently, for each occurrence, selected from H, OH, C₁-C₆ alkyl, C₁-C₆ haloalkyl, C₁-C₈ alkylamine, C₁-C₈ alkyl-OH, C(O)H, C(O)(C₁-C₆ alkyl), C(O)OH, C(O)O(C₁-C₆ alkyl), (C₁-C₆ alkyl)C(O)OH, (C₁-C₆ alkyl)C(O)O(C₁-C₆ alkyl), (C₁-C₆ alkyl)CO(C₁-C₆ alkyl), (C₁-C₆ alkyl)CS(C₁-C₆ alkyl), C(O)N(R″)₂, N(R″)₂, and N(R″)₃⁺; and
R″ is independently, for each occurrence, selected from H, C₁-C₆ alkyl, C₁-C₆ haloalkyl, (C₁-C₆ alkyl)C(O)OH, and;
wherein either the cyclic peptide does not comprise a Chelator or comprises one Chelator;
when a variable group R¹, R², R⁴, R⁵, R⁶, R⁷, R⁸, R⁹, R¹⁰, R¹², R¹³, or R¹⁵ is the amino acid side chain of a cyclic amino acid, the corresponding amino acid nitrogen of the peptide backbone forms part of the cyclic group;
each alpha-carbon atom on the peptide backbone is optionally substituted with methyl; and
wherein the cyclic peptide optionally comprises a radionuclide.

18. The cyclic peptide of claim 17, or a pharmaceutically acceptable salt thereof, wherein the C-Terminus is-L²-Chelator.

19-21. (canceled)

22. The cyclic peptide of claim 17, or a pharmaceutically acceptable salt thereof, wherein P¹ is-L¹-Chelator.

23-33. (canceled)

34. The cyclic peptide of claim 1, or a pharmaceutically acceptable salt thereof, wherein Chelator is independently selected from a group consisting of ethylenediamine tetraacetic acid (EDTA), diethylenetriamine pentaacetic acid (DTPA), 1,4,7,10-tetra-azacylcododecane-N,N′,N″,N″-tetraacetic acid (DOTA), 6-((16-((6-Carboxypyridin-2-yl)methyl)-1,4,10,13-tetraoxa-7, 16-diazacyclooctadecan-7-yl)methyl)-4-isothiocyanatopicolinic acid (Macropa), Macrodipa, 2,2′,2″,2″-(1,10-dioxa-4,7,13,16-tetraazacyclooctadecane-4,7,13,16-tetrayl)tetraacetic acid) (Crown), 1,4,7,10-Tetraazacyclododecane-1,4,7,10-tetraacetic acid, α-(2-carboxyethyl) (DOTAGA), 1,4,7-Triazacyclononane-N,N′,N″-triacetic acid (NOTA), 1,4,7,10-tetraazacyclododecane-N, N′,N″,N″-tetraacetic acid (TETA), 1,4,7,10,13-pentaazacyclopentadecane-N,N′,N″,N″,N″-pentaacetic acid (PEPA), 1,4,7,10,13,16-hexaazacyclohexadecane-N,N′,N″,N″,N″,N″-hexaacetic acid (HEHA), N′-[5-(Acetyl-hydroxy-amino)pentyl]-N-[5-[3-(5-aminopentyl-hydroxy-carbamoyl) propanoylamino]pentyl]-N-hydroxy-butane diamide (DFO), and 1-(1-carboxy-3-carboxypropyl)-4,7-bis-(carboxymethyl)-1,4,7-triazacyclononane (NODAGA).

35. (canceled)

36. The cyclic peptide of claim 1, or a pharmaceutically acceptable salt thereof, wherein the cyclic peptide of Formula I or Formula V is selected from

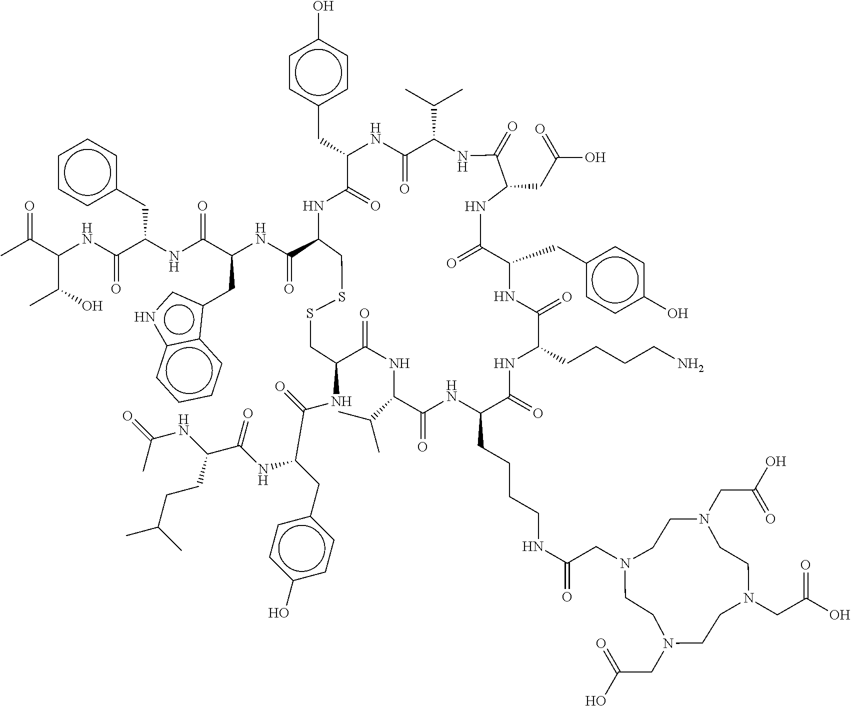

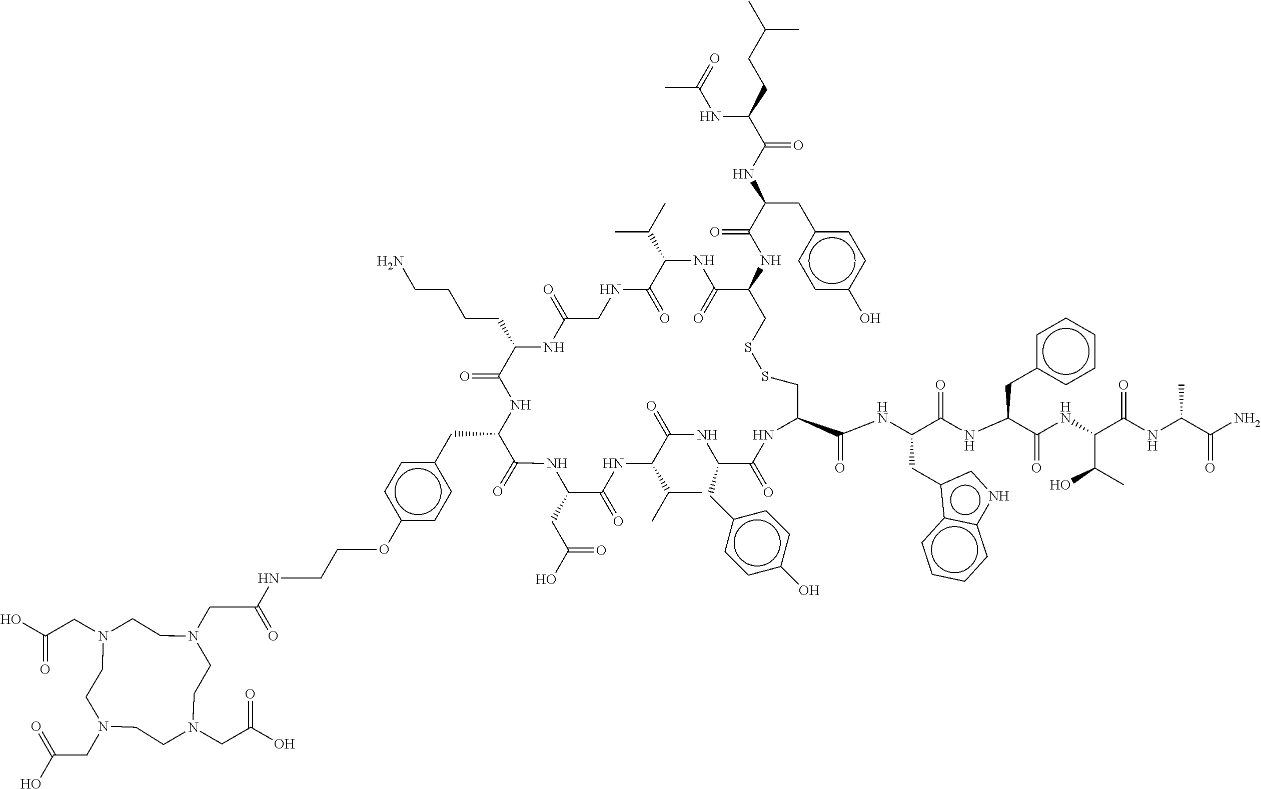

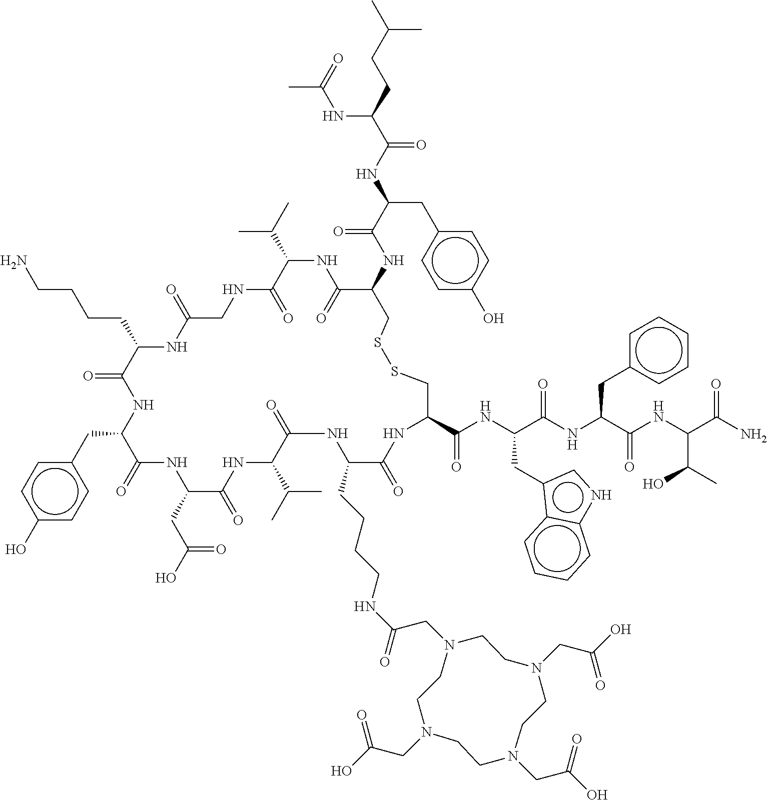

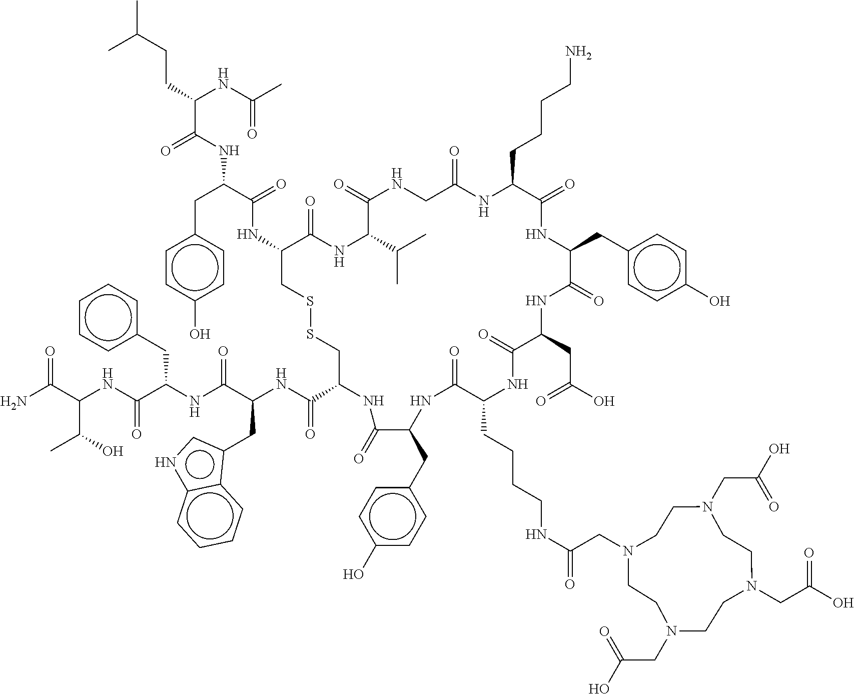

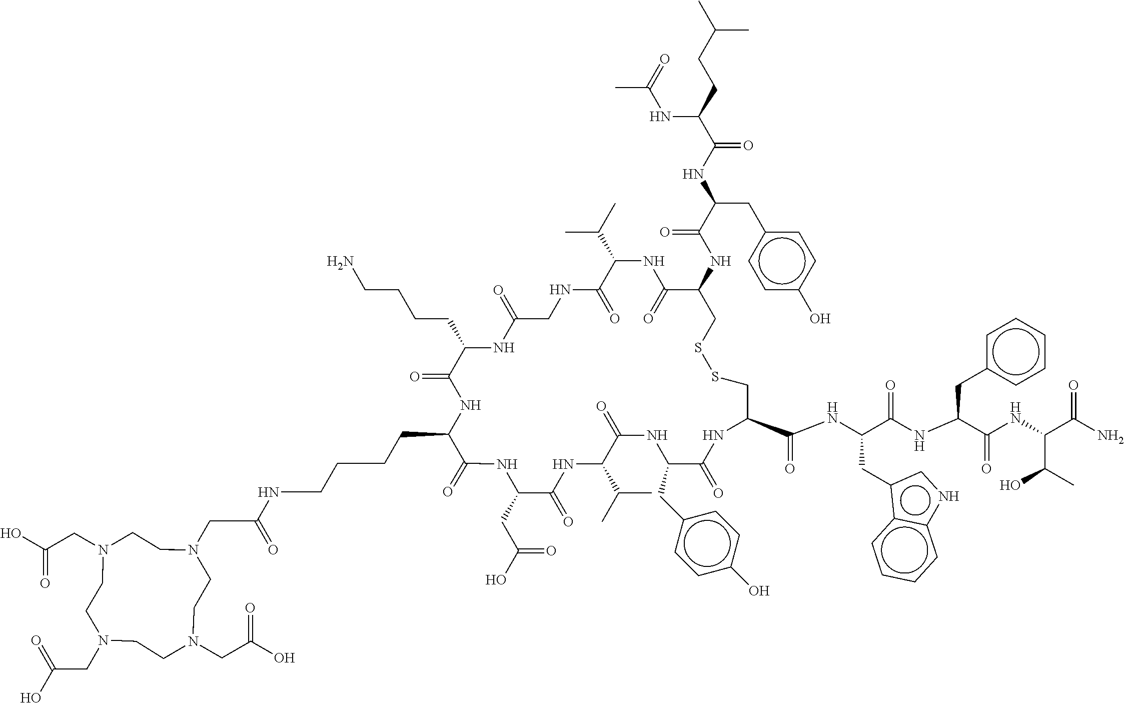

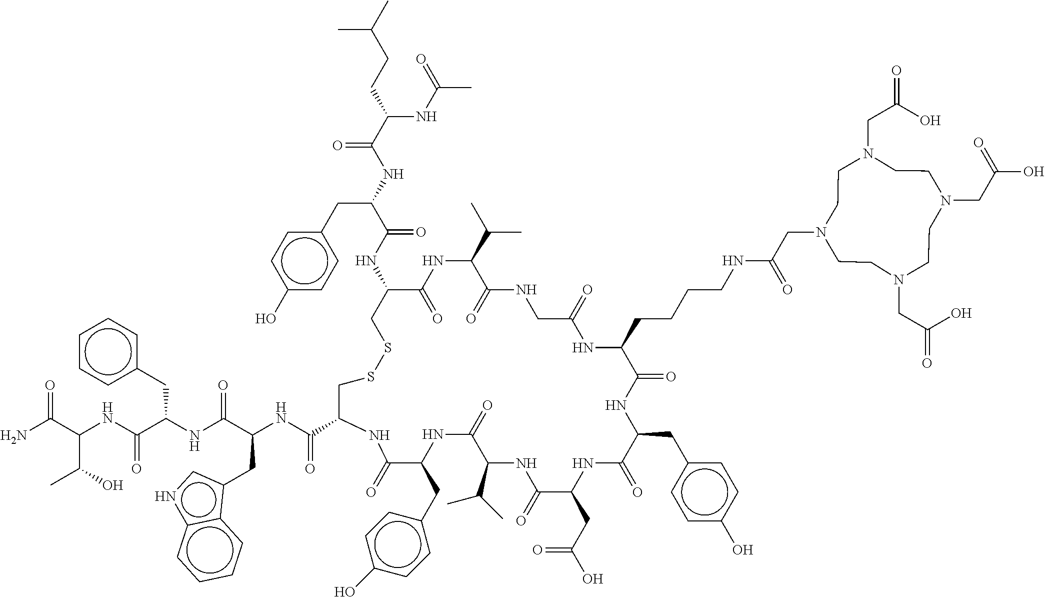

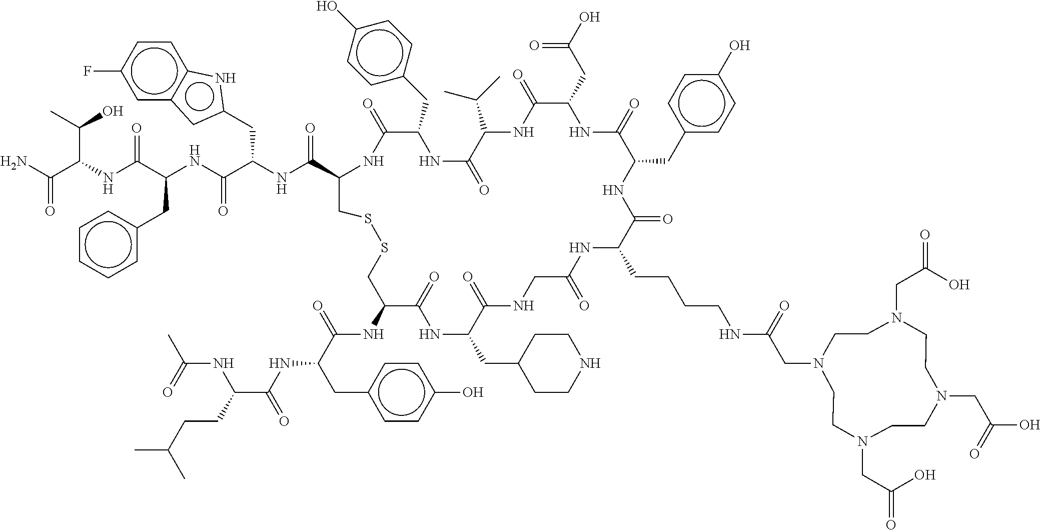

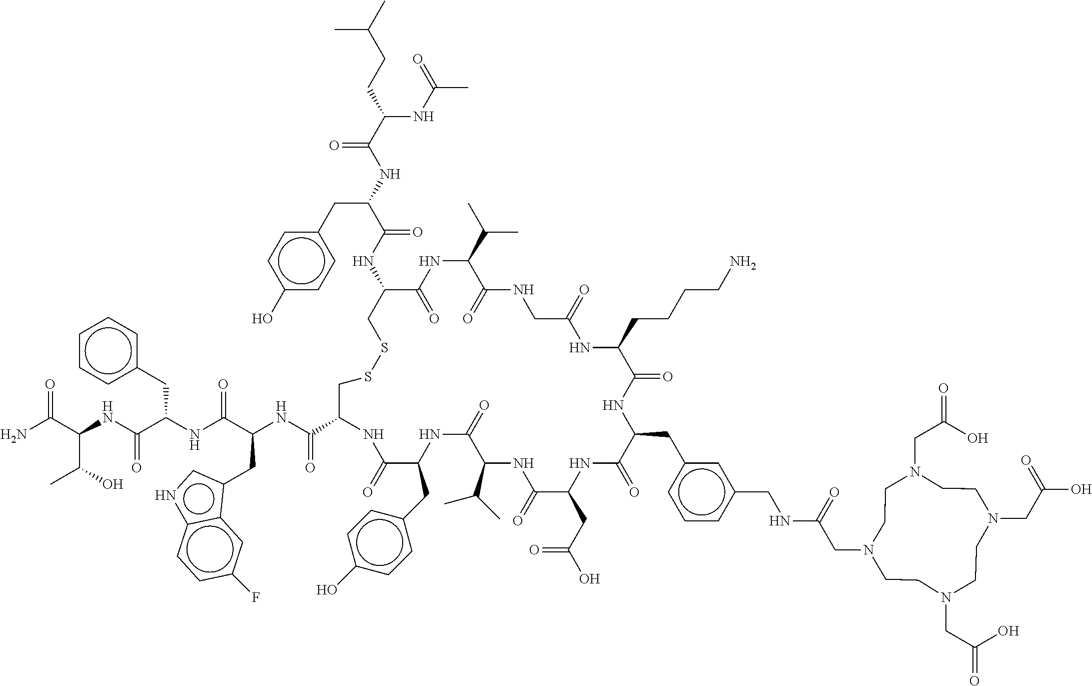

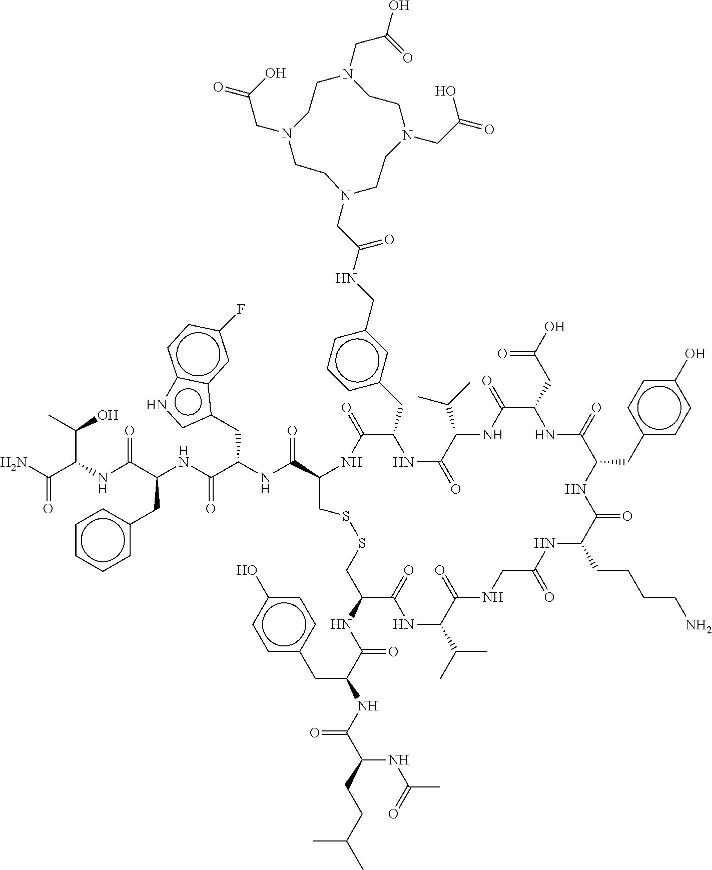

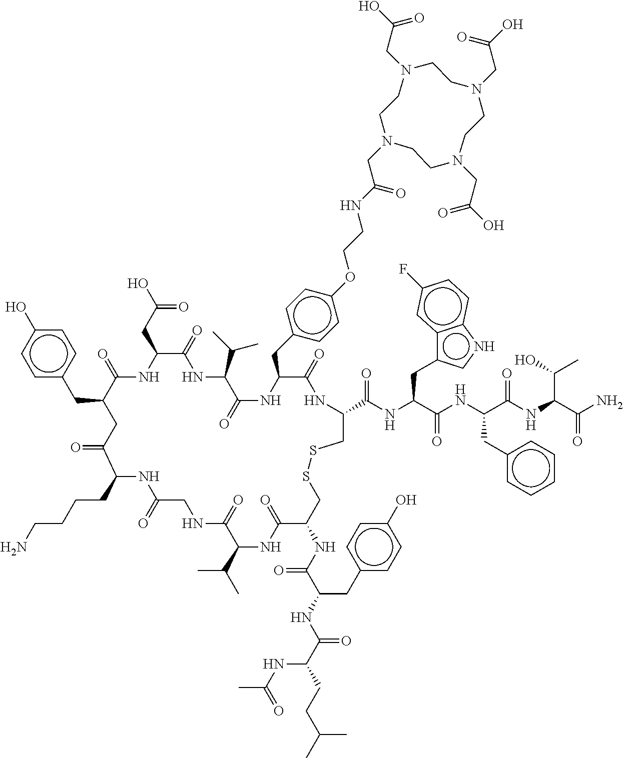

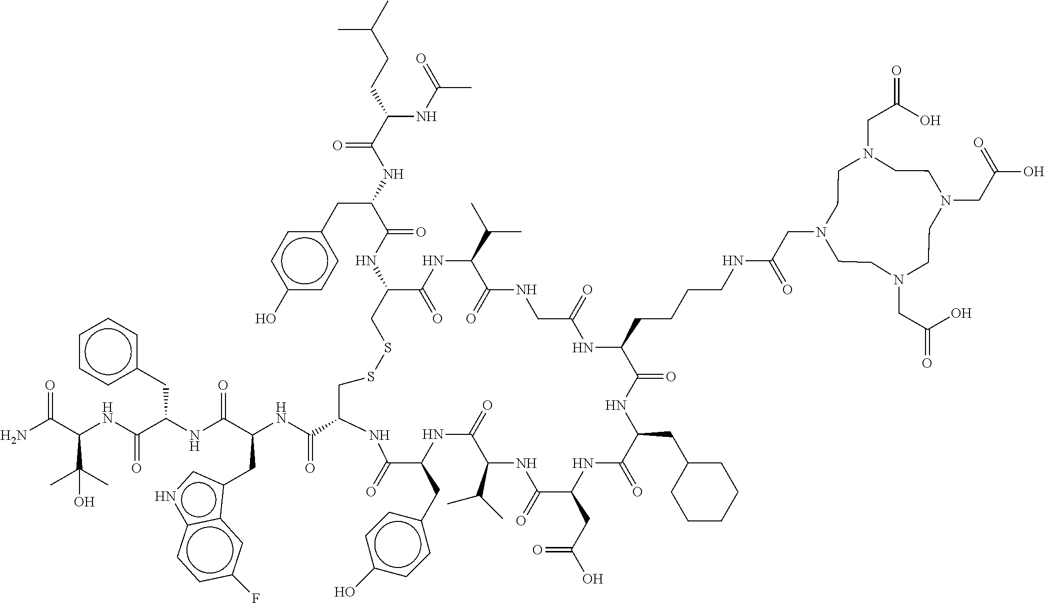

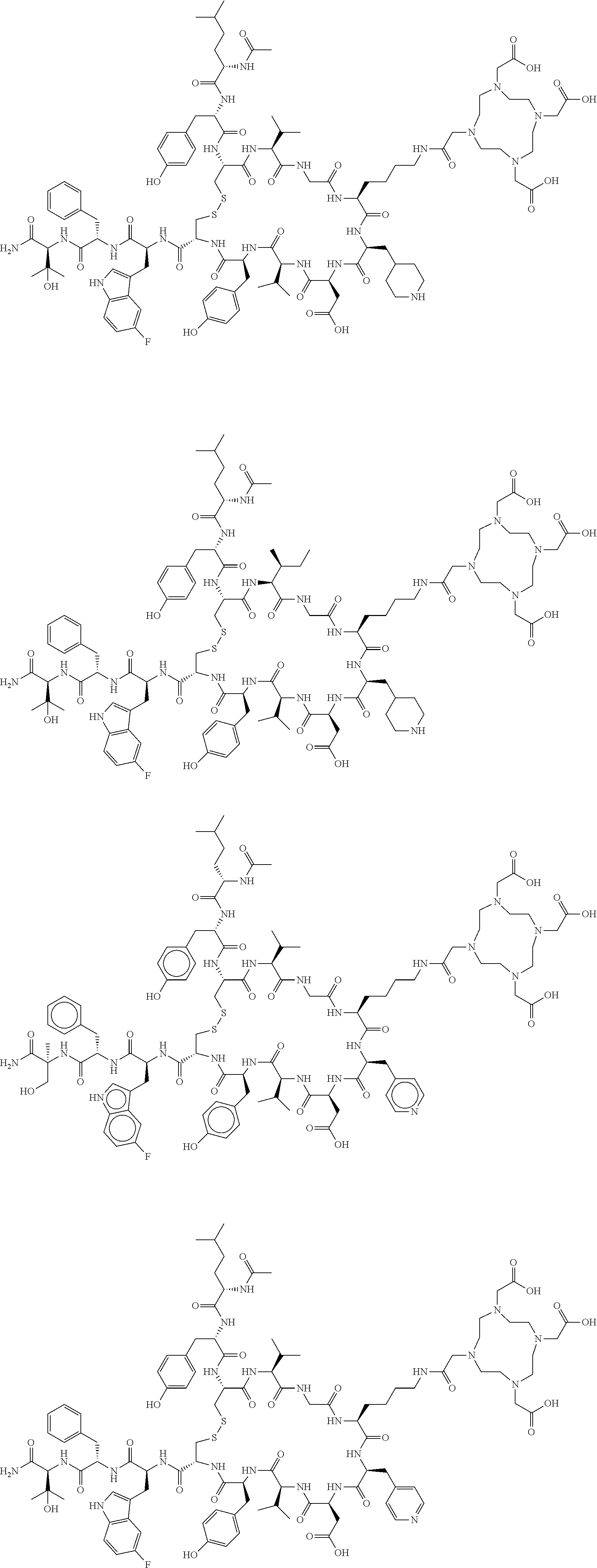

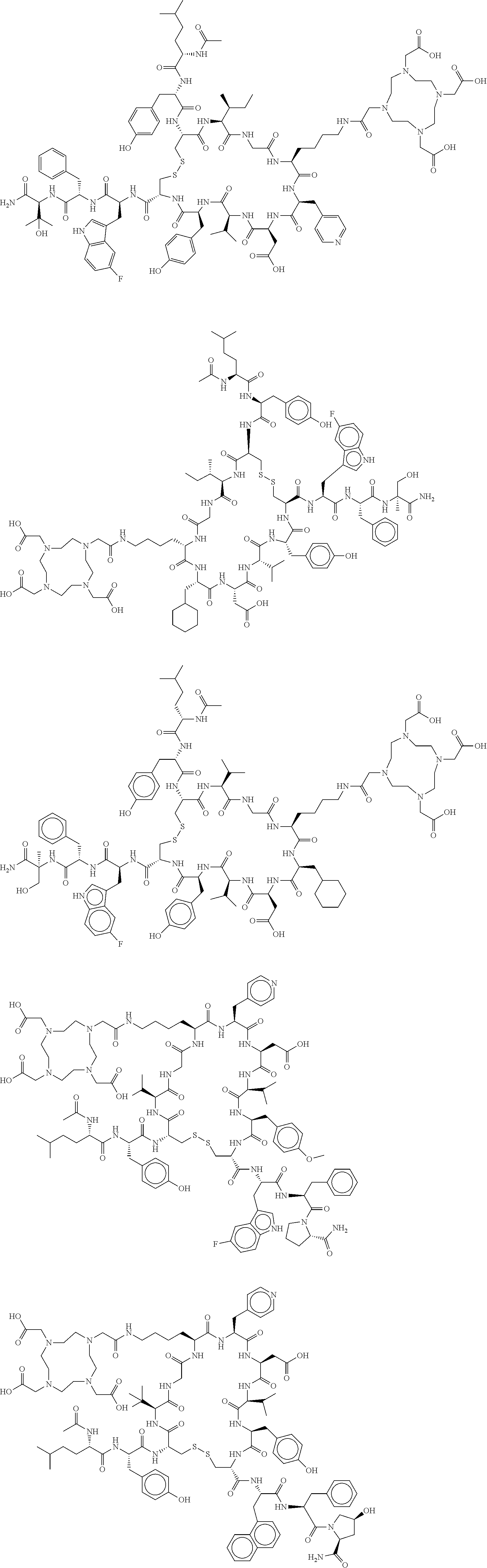

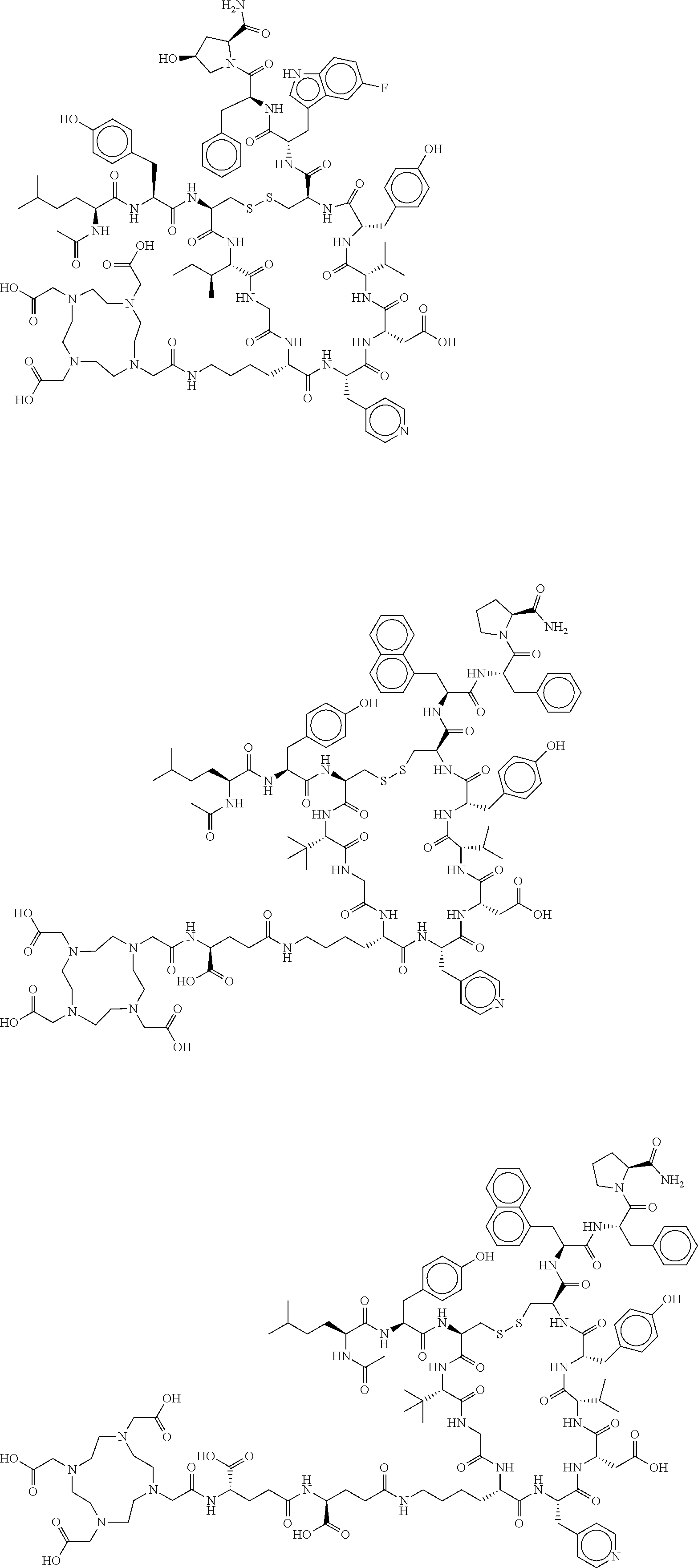

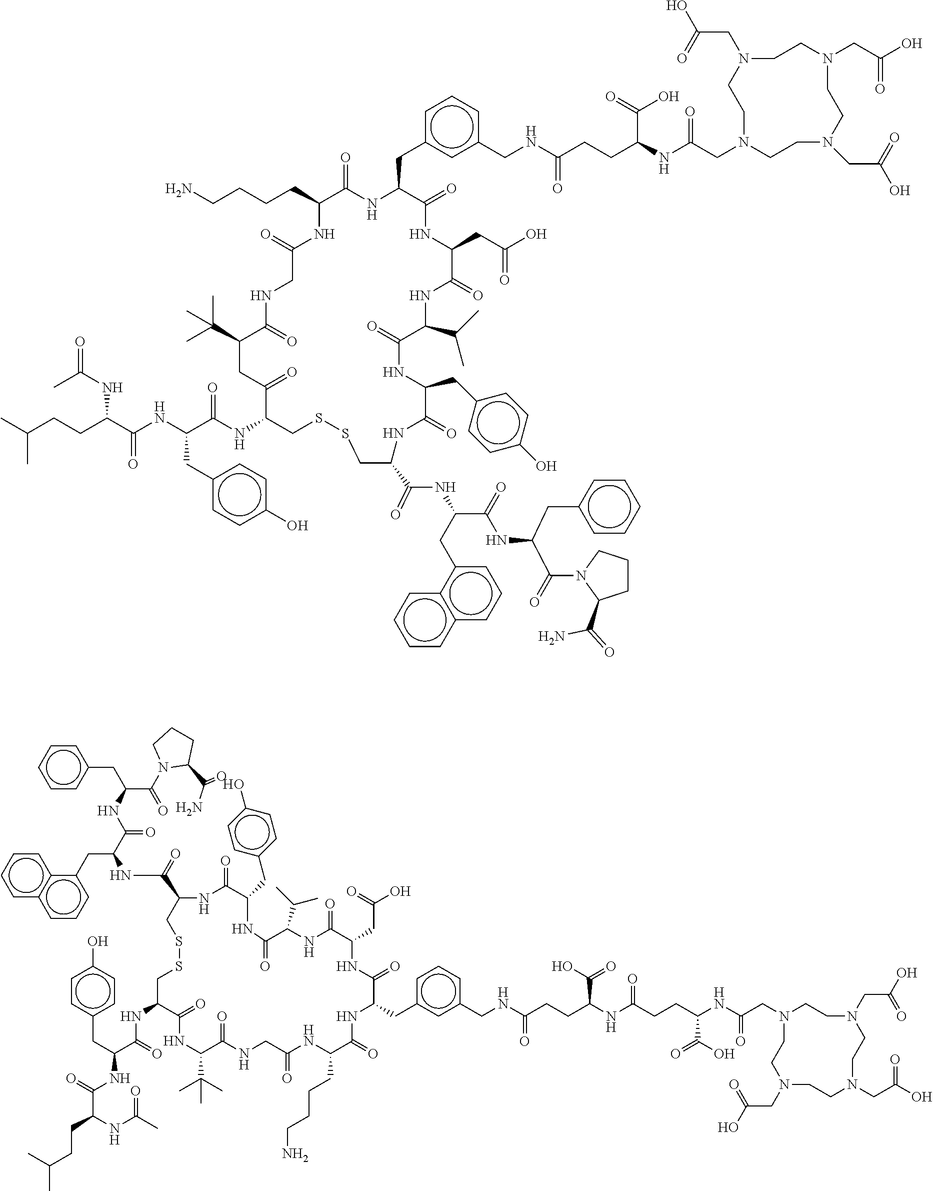

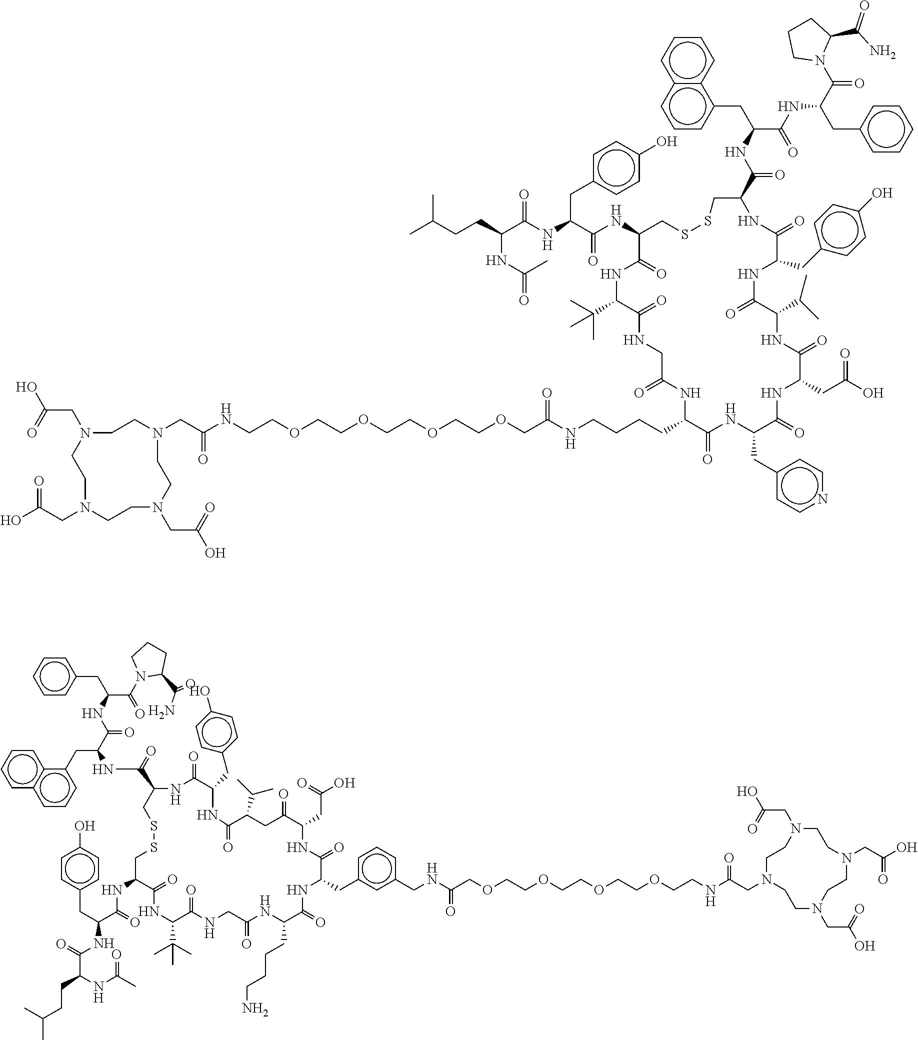

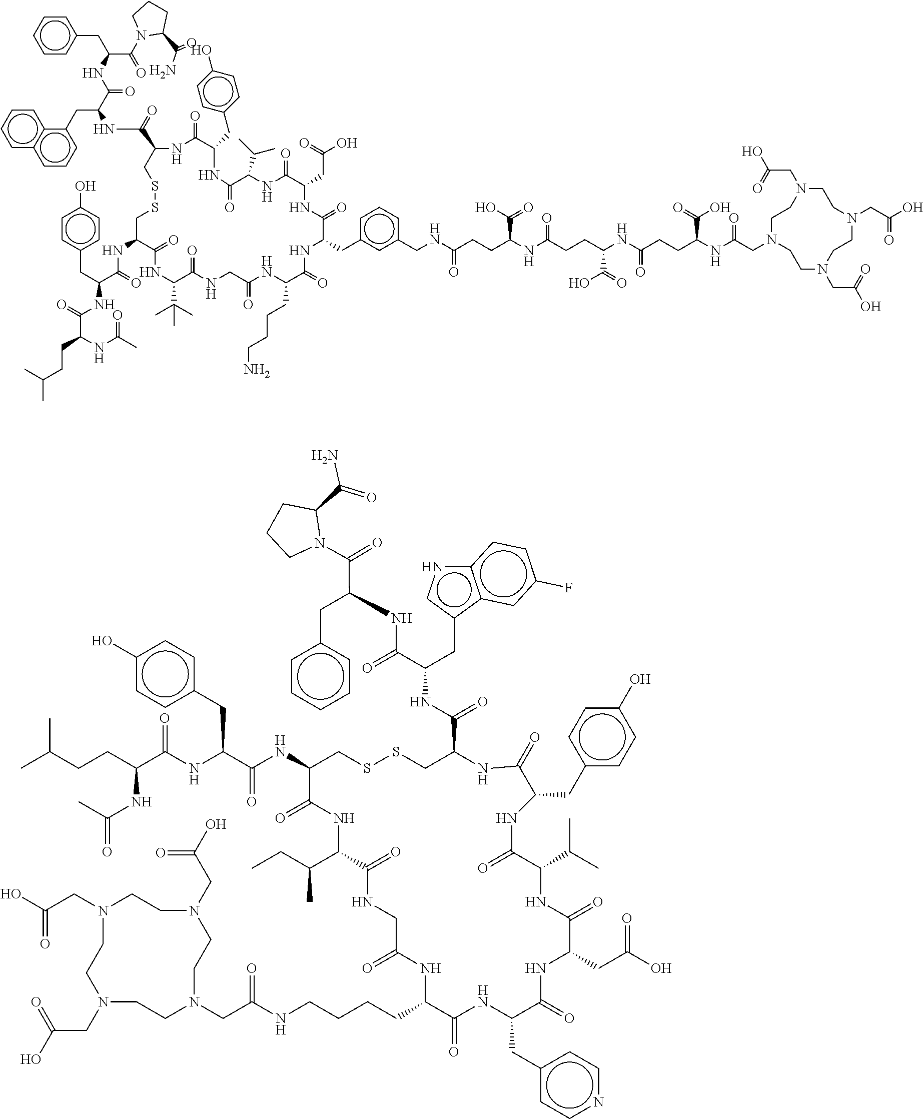

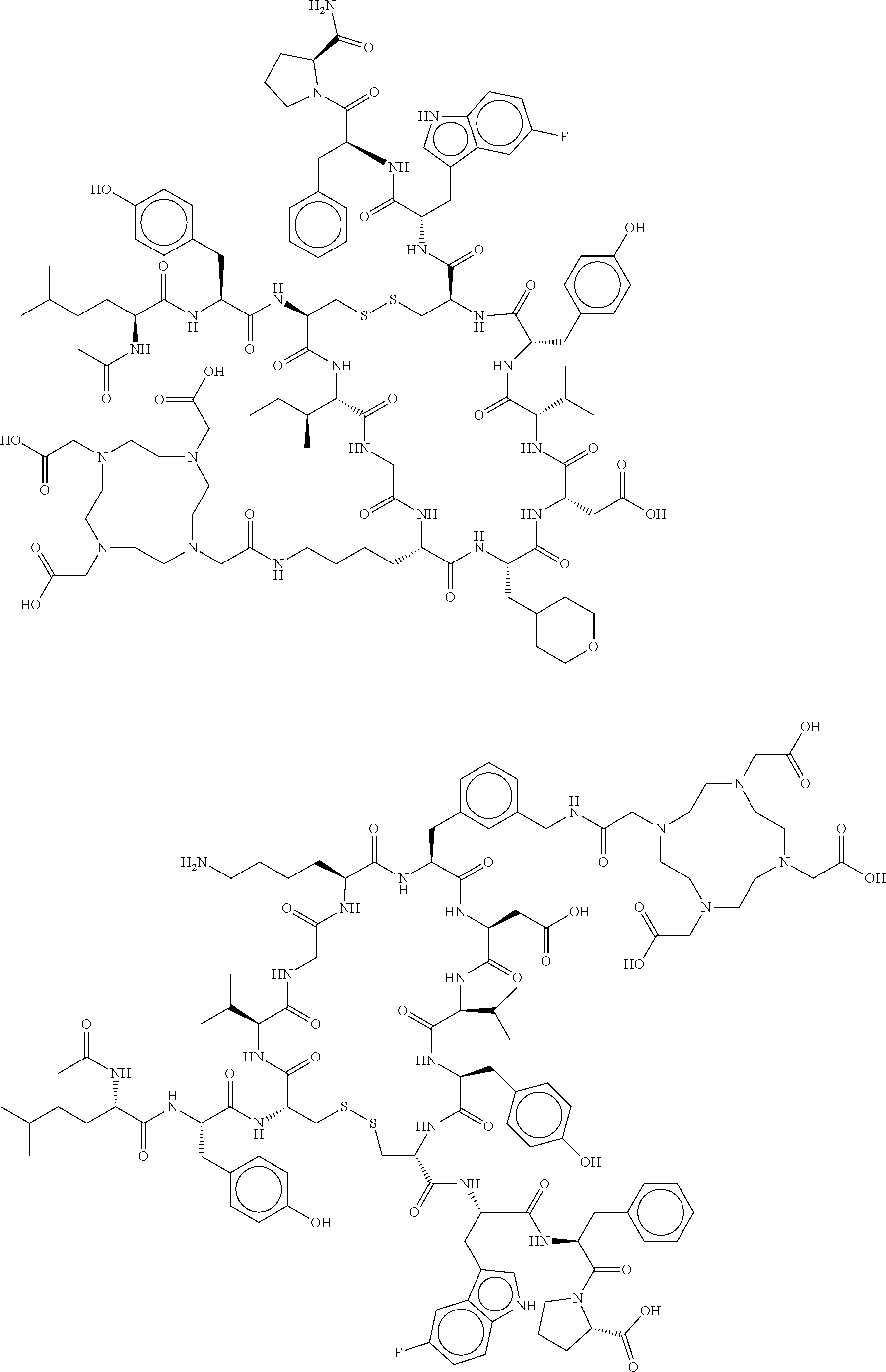

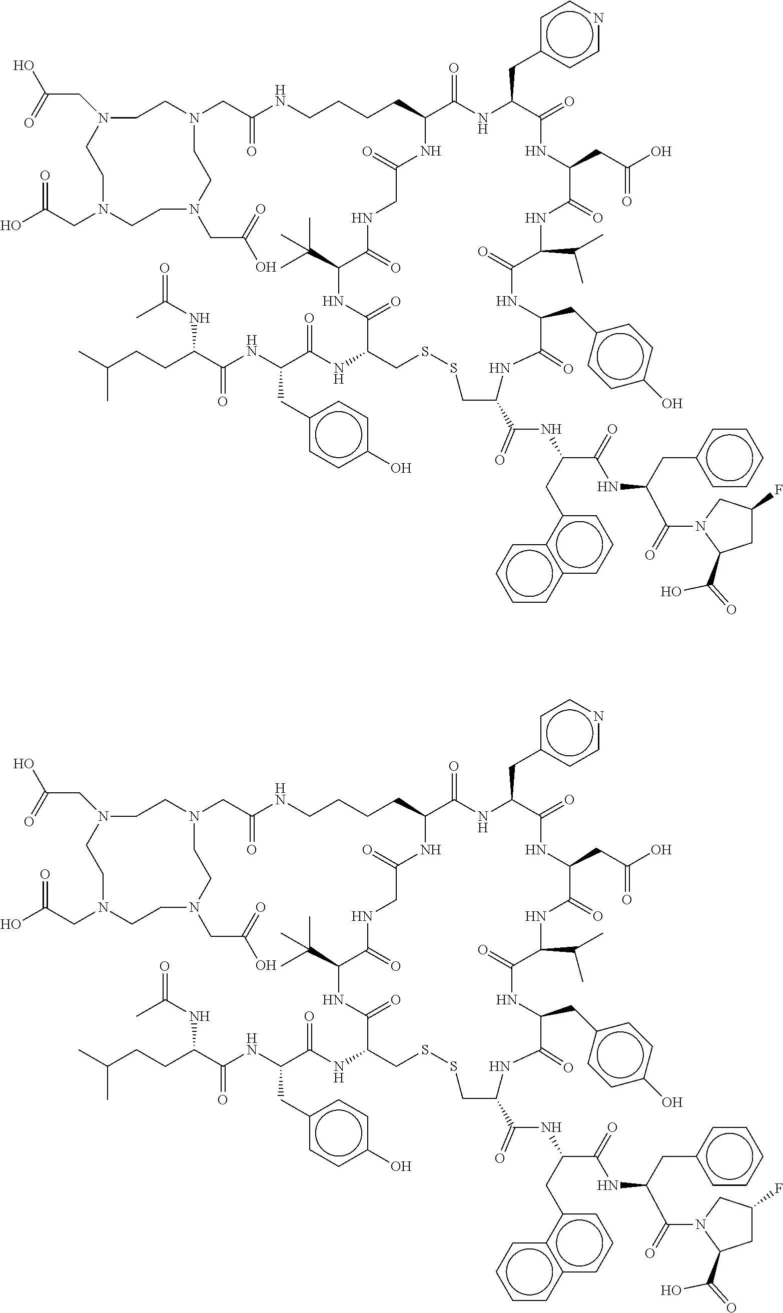

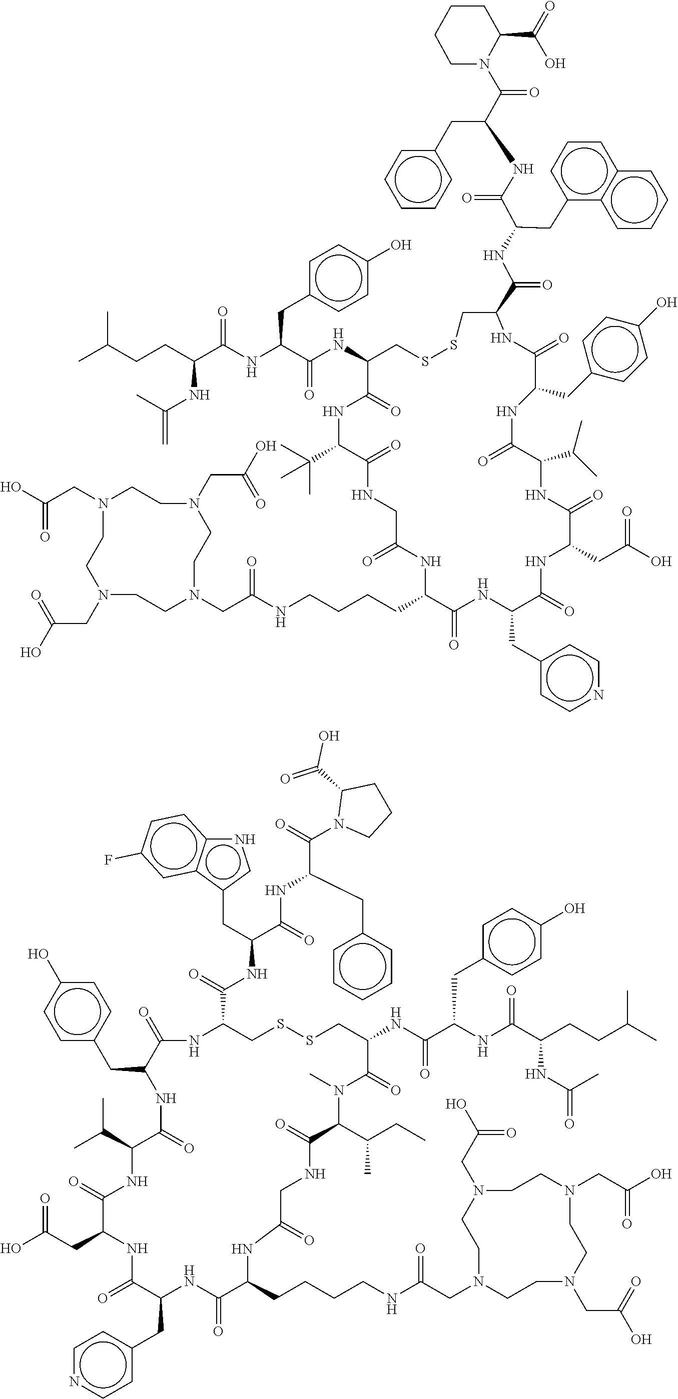

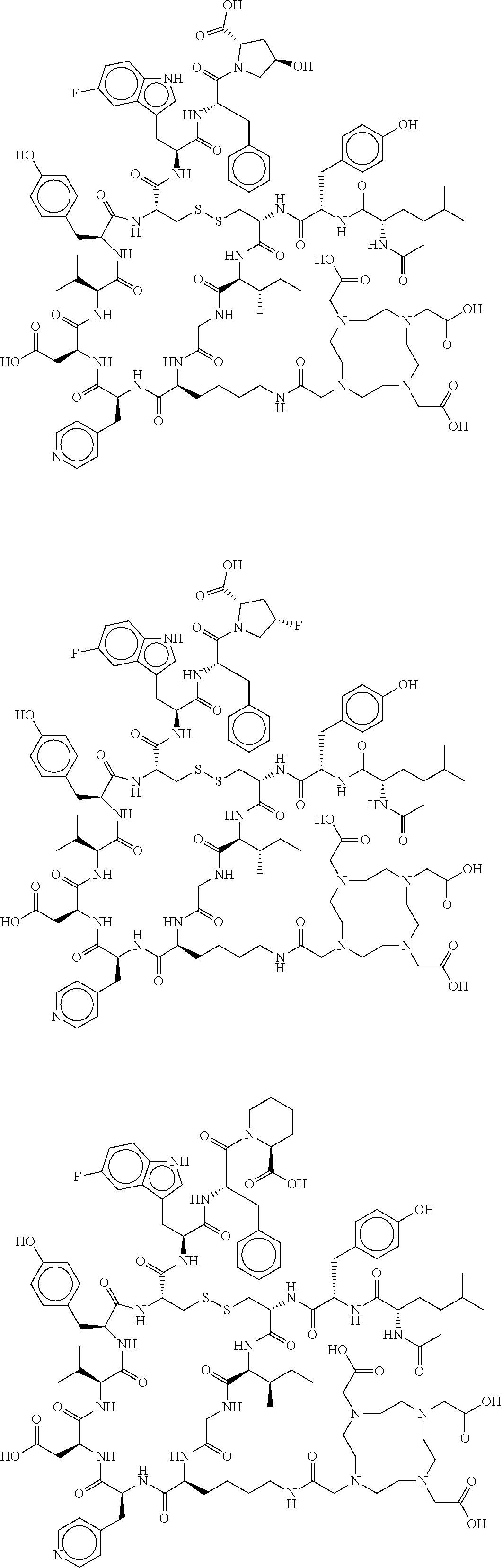

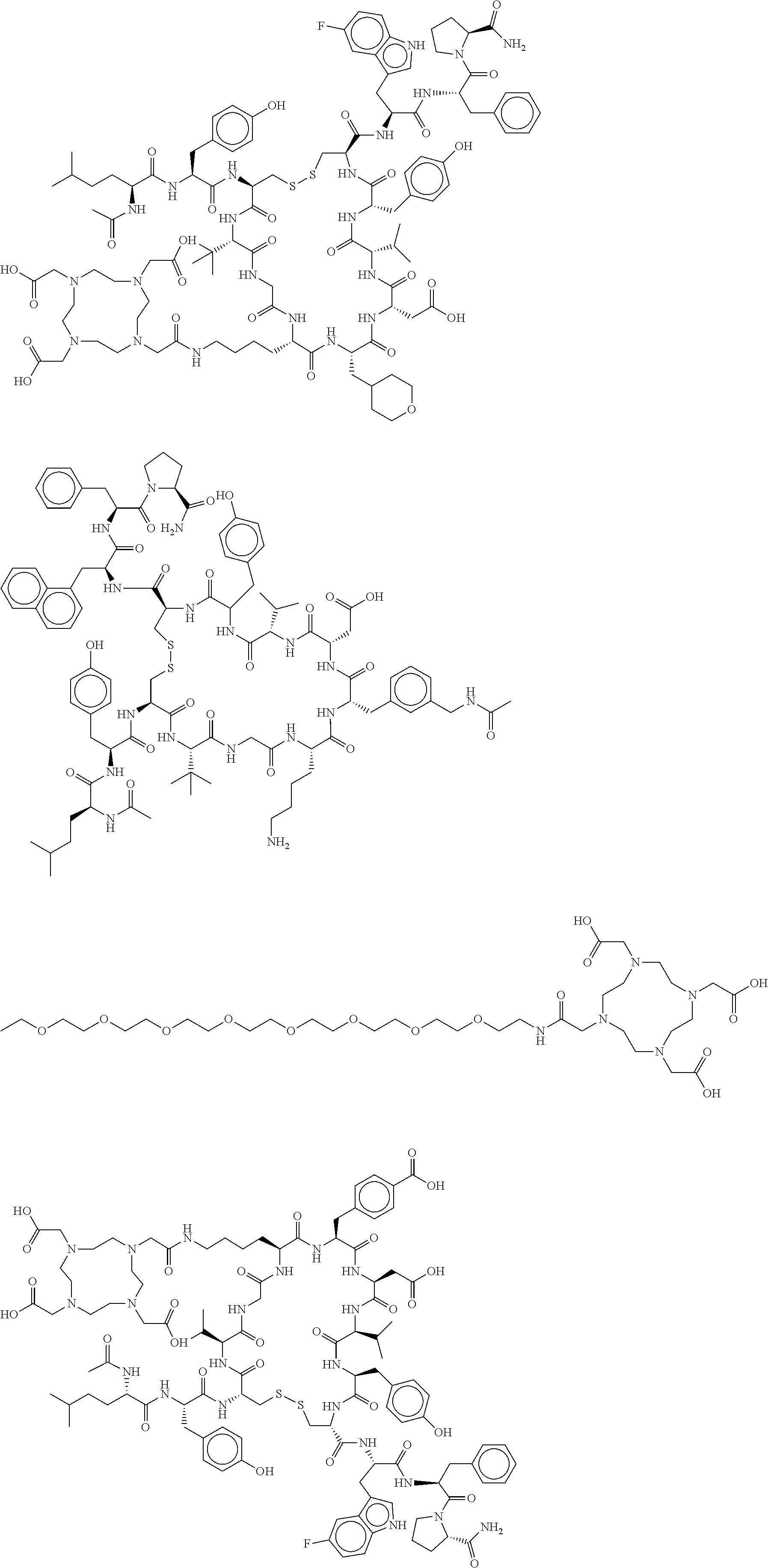

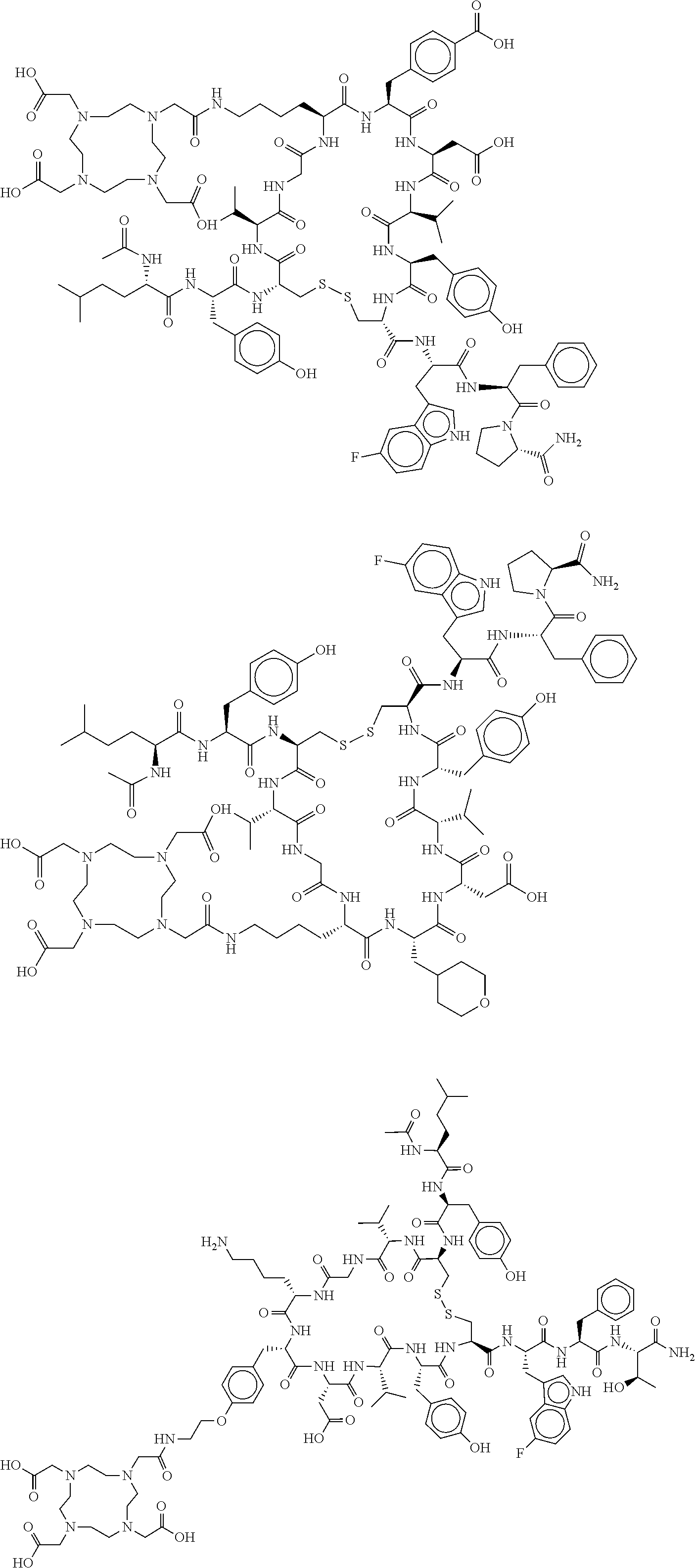

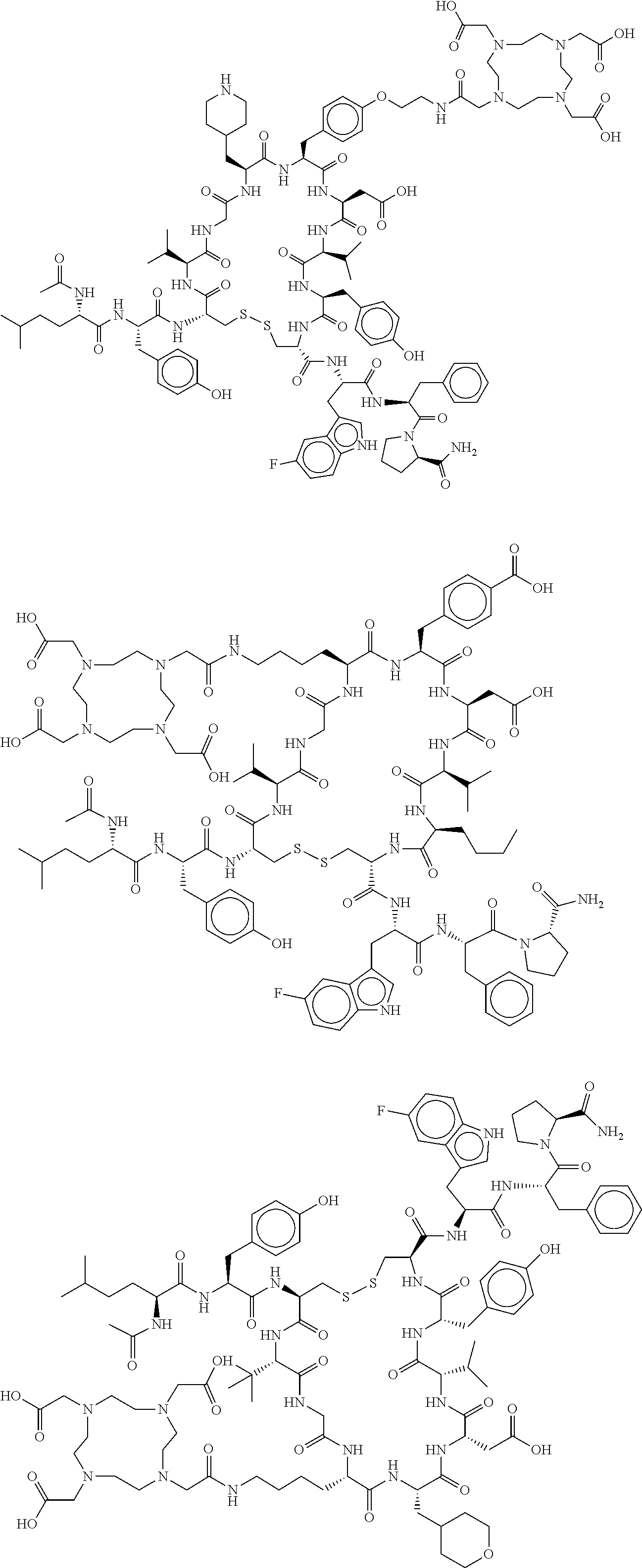

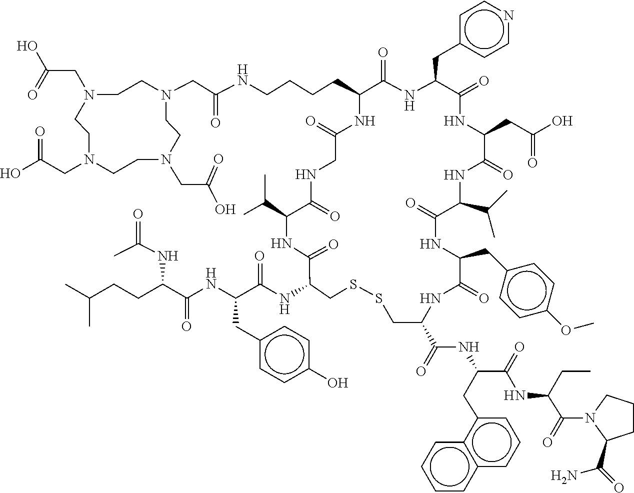

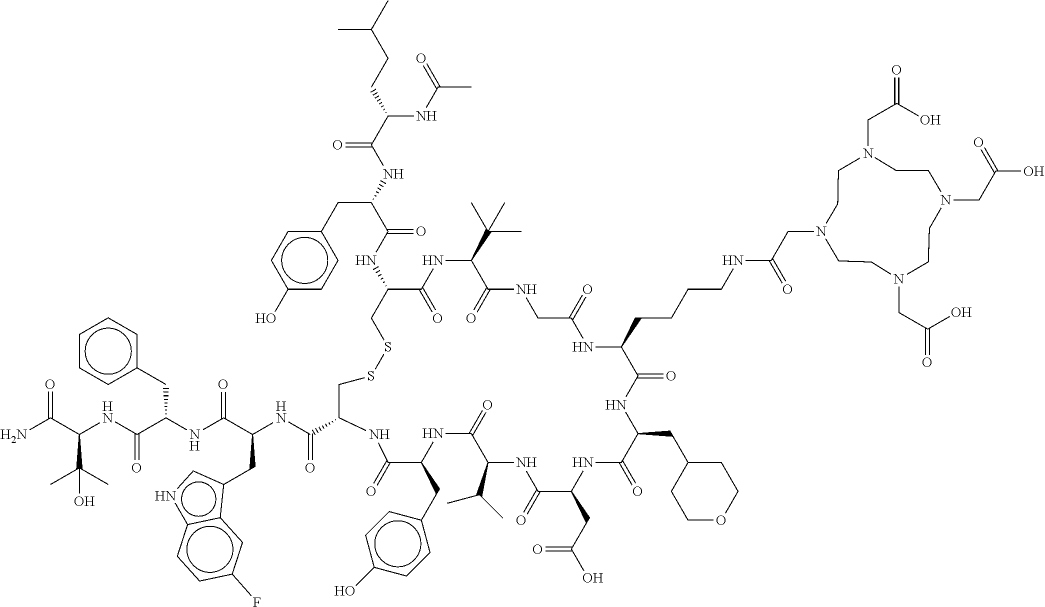

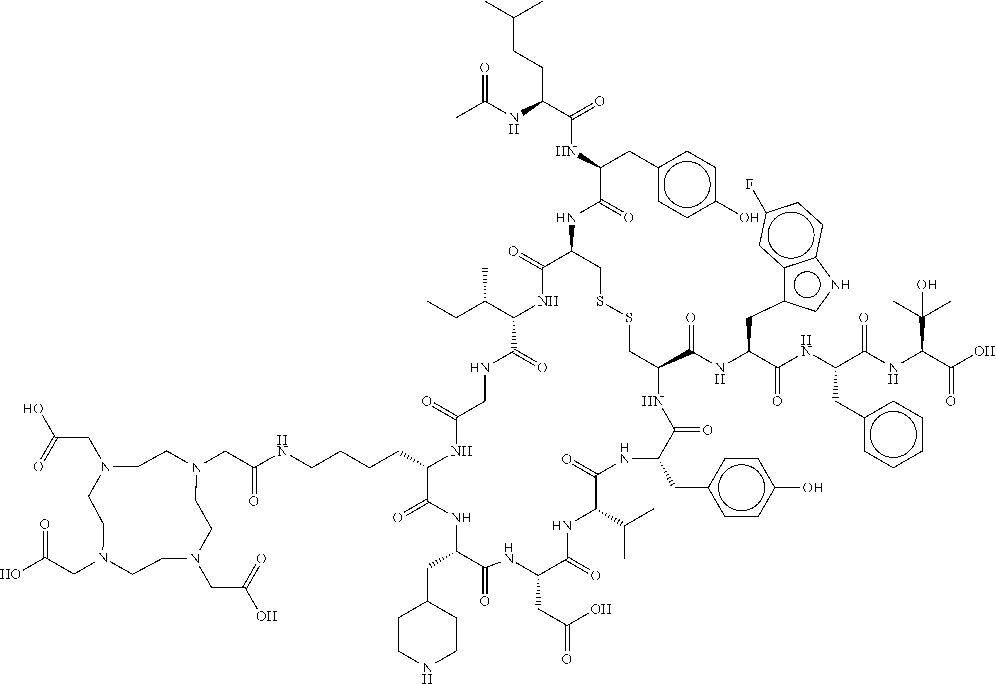

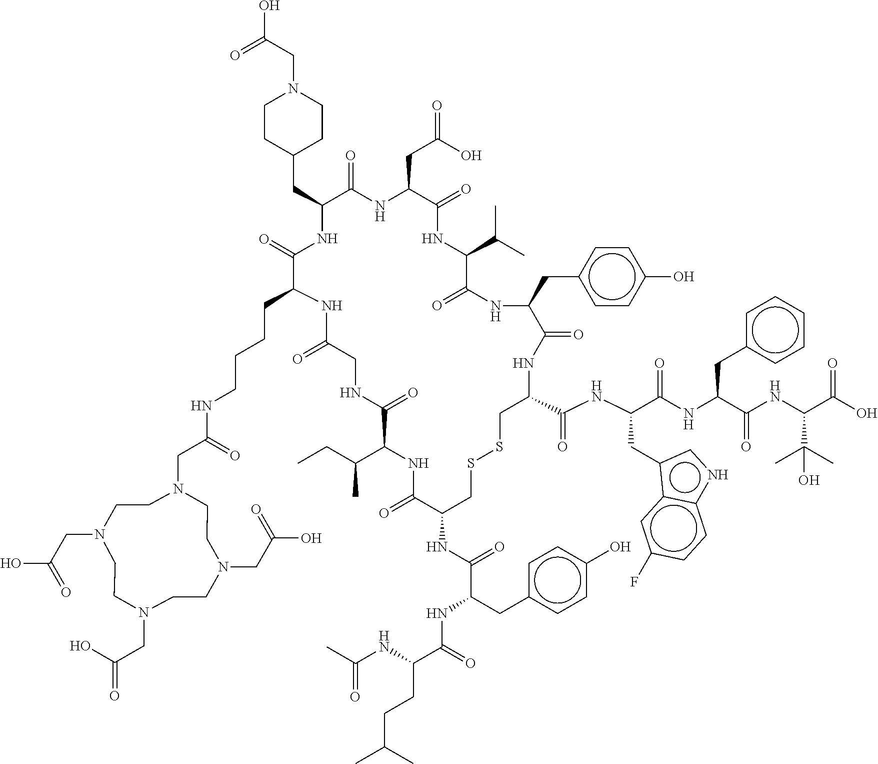

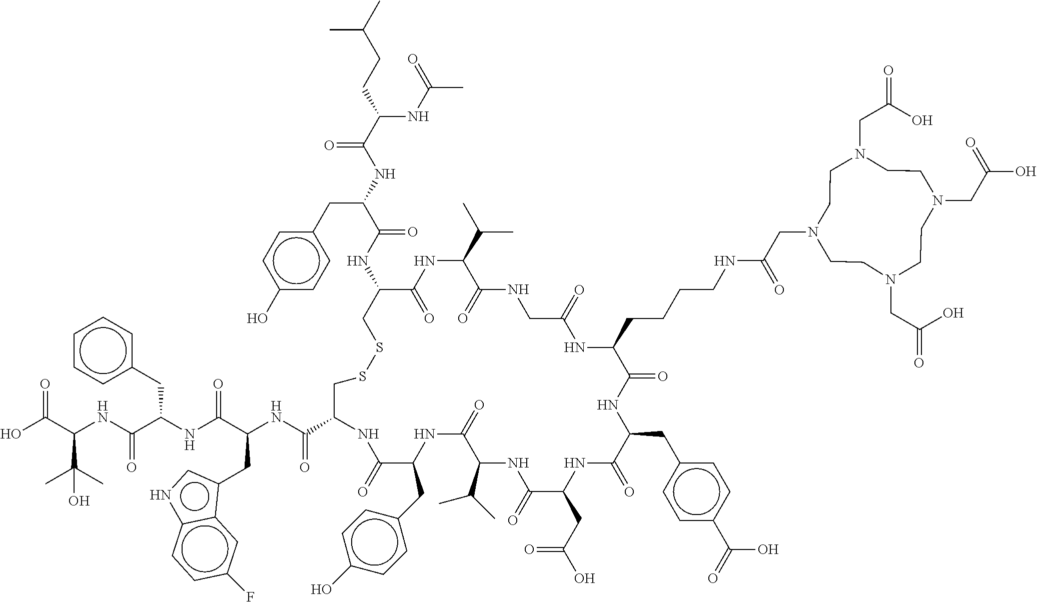

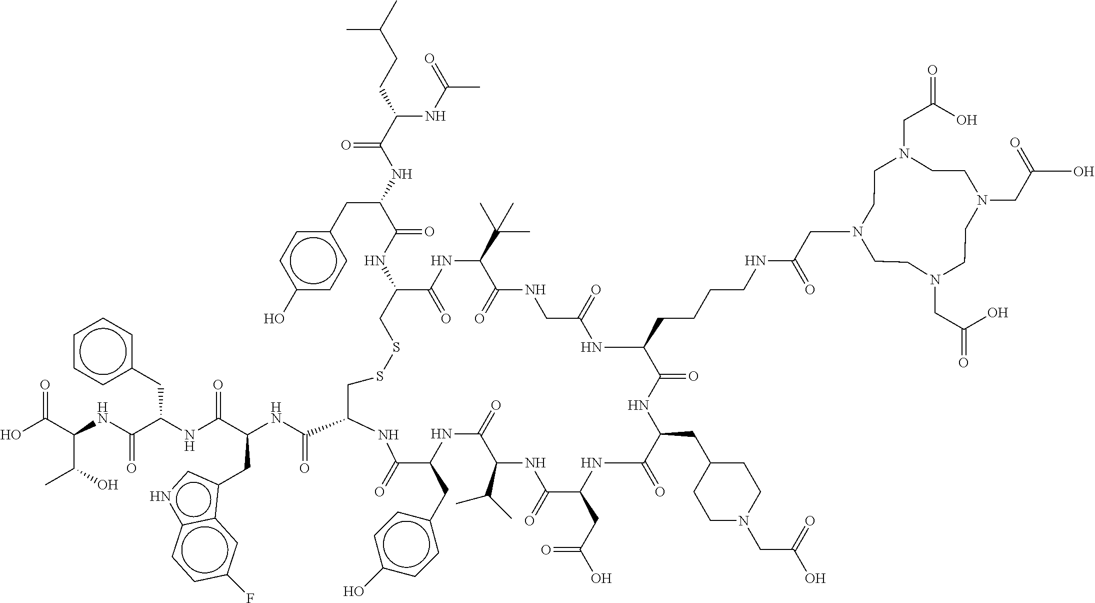

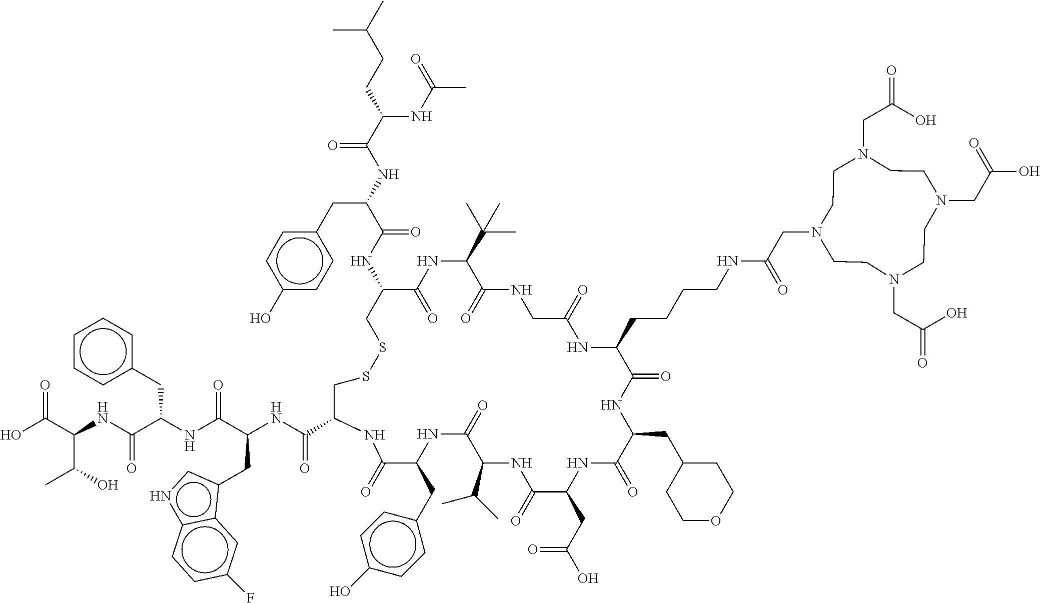

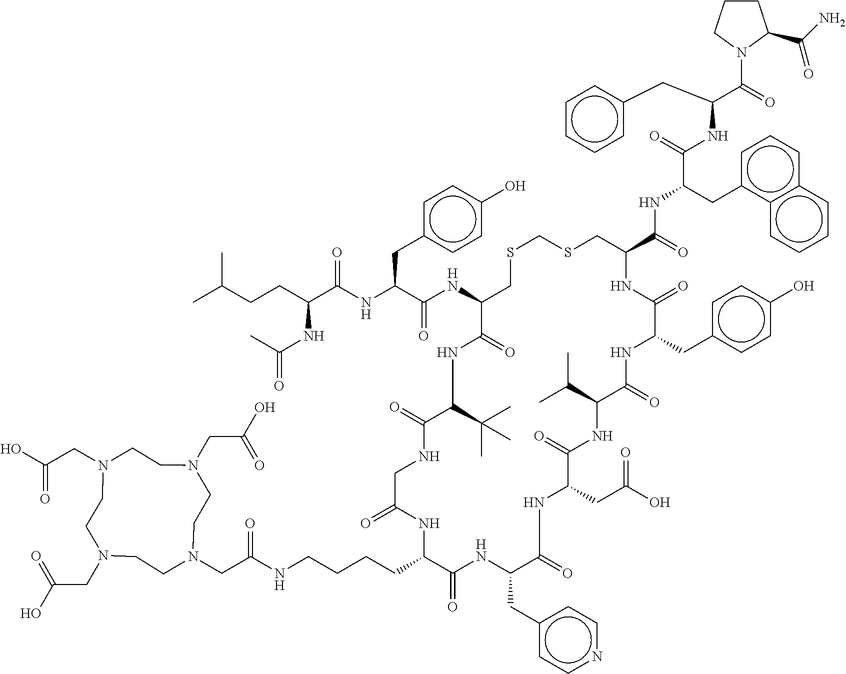

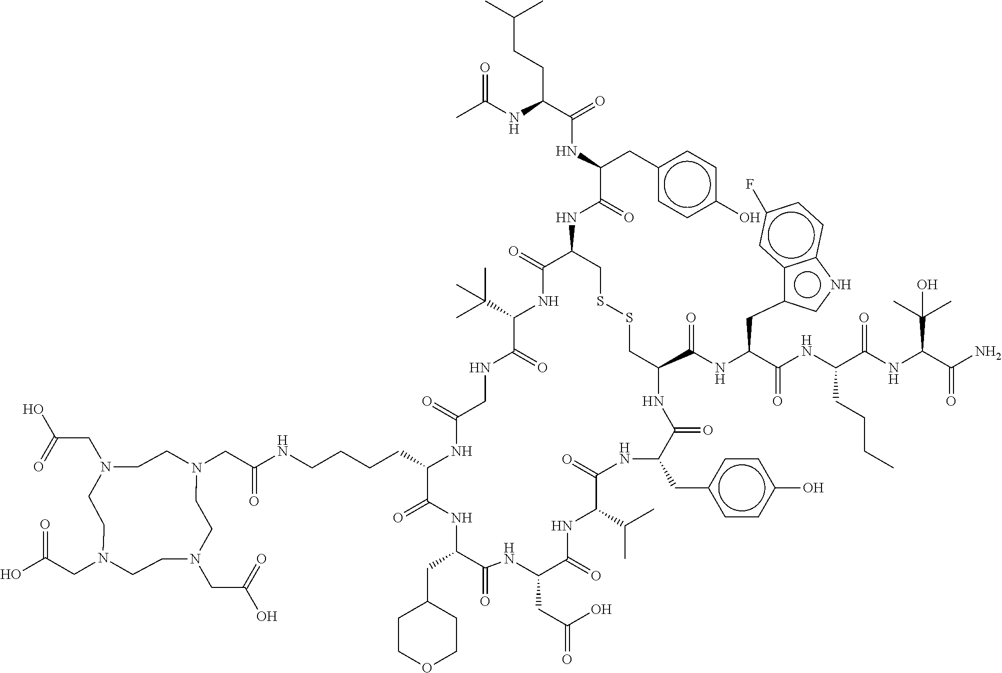

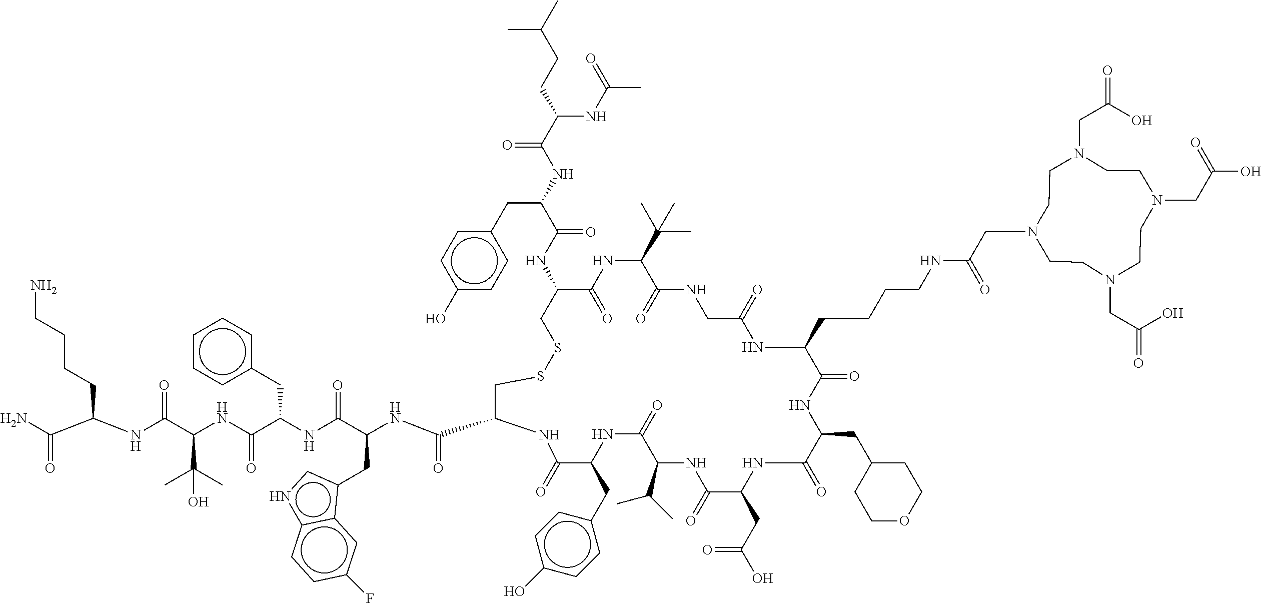

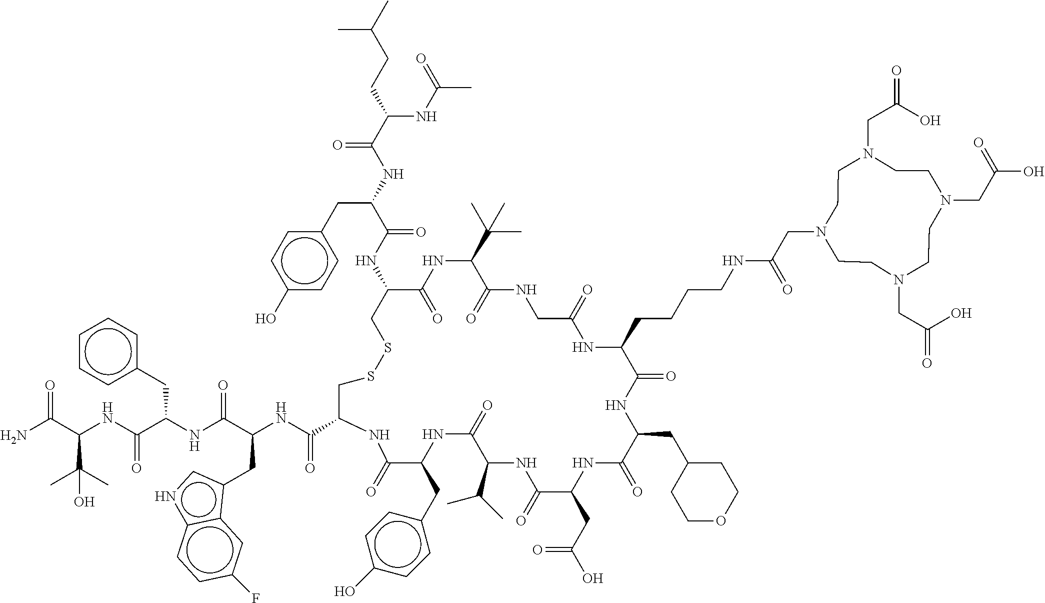

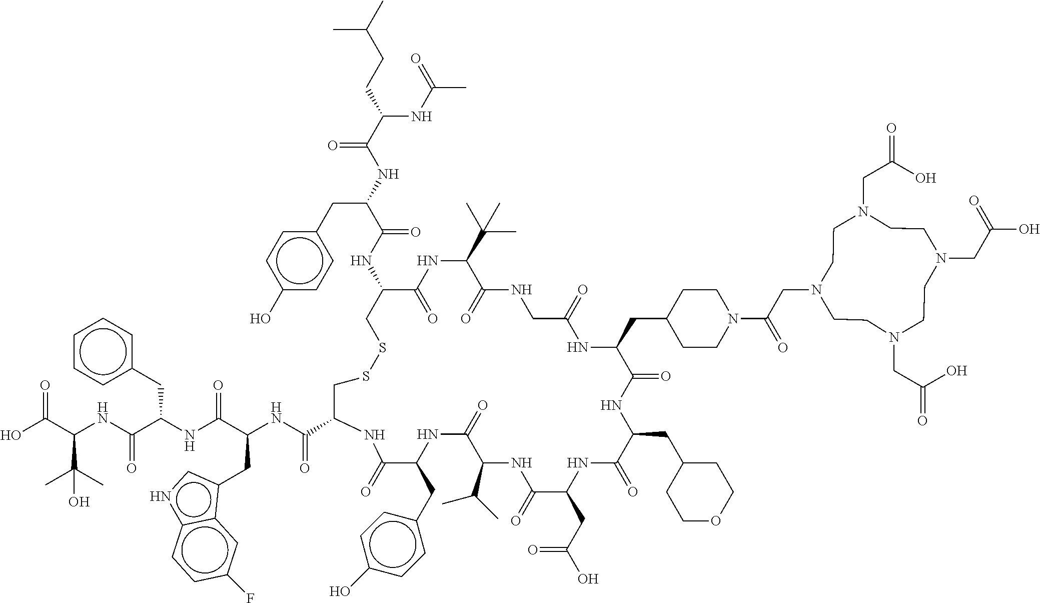

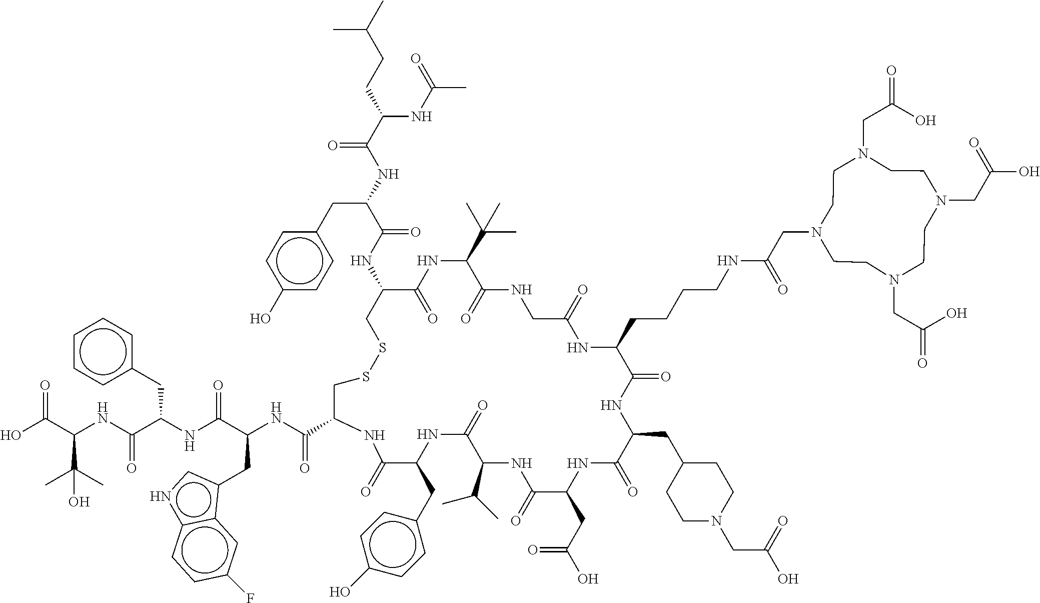

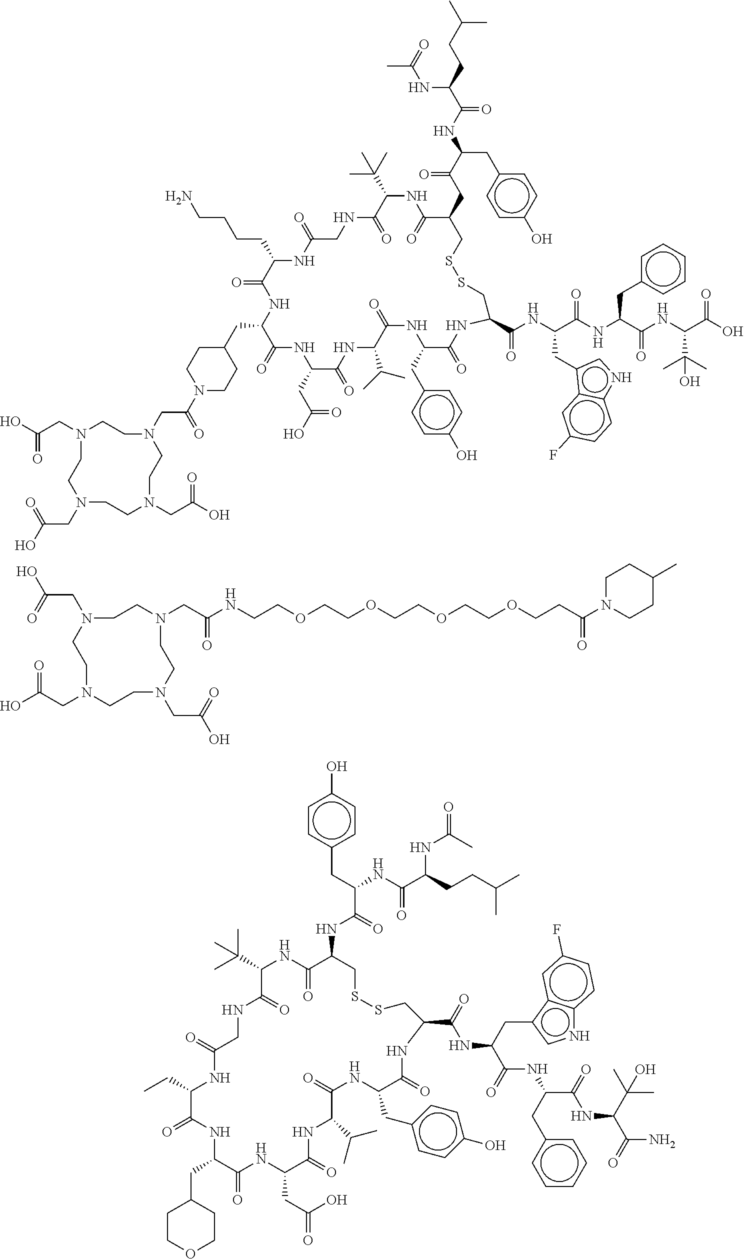

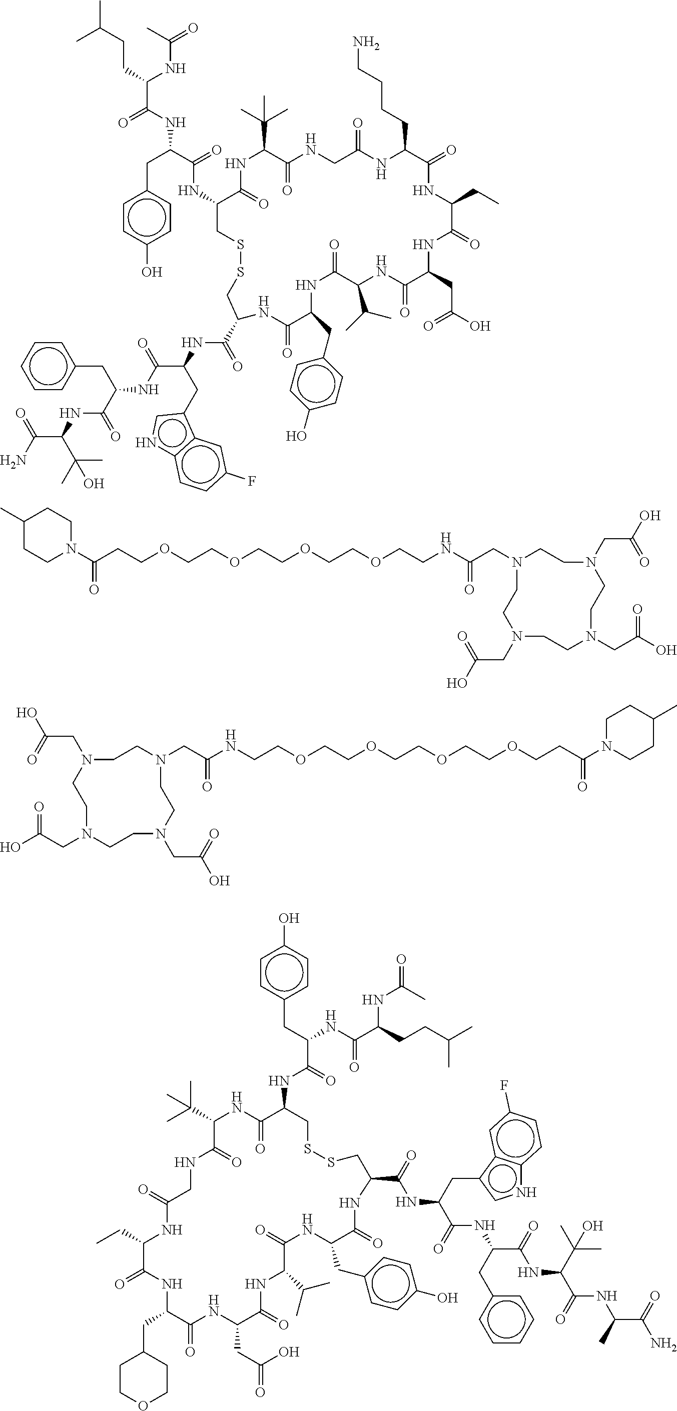

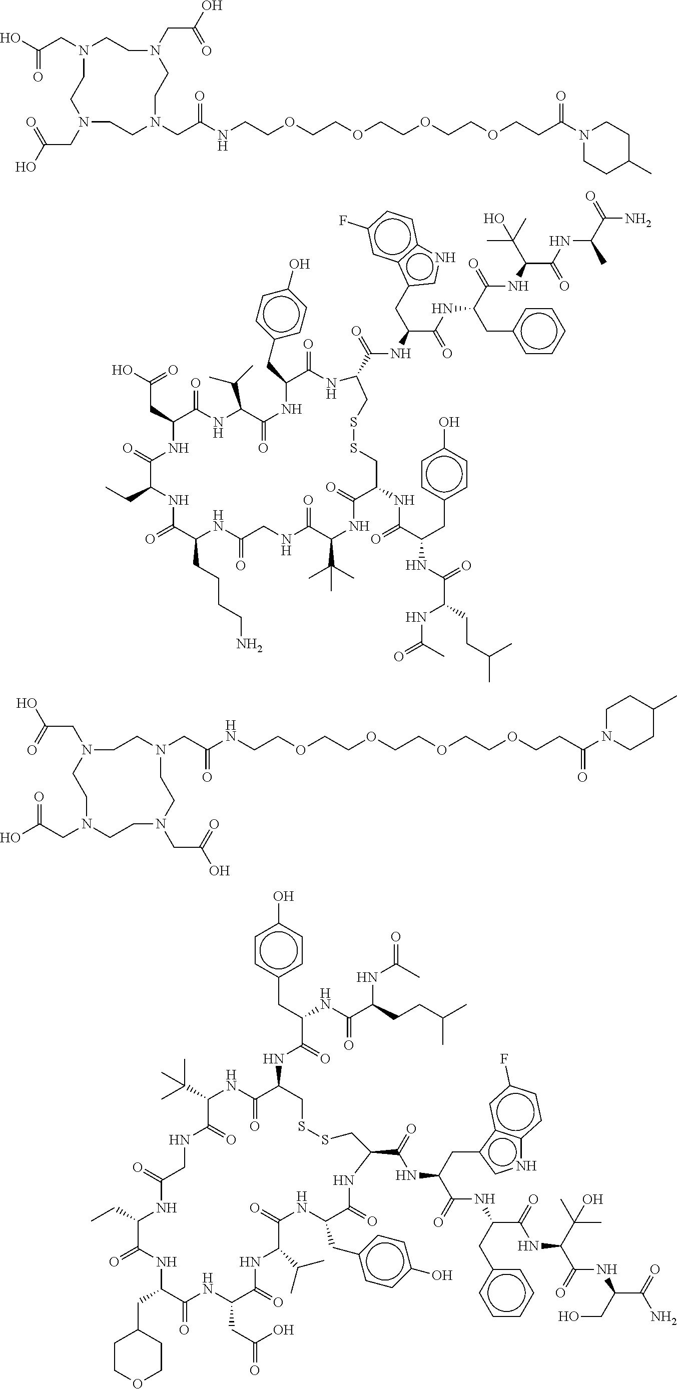

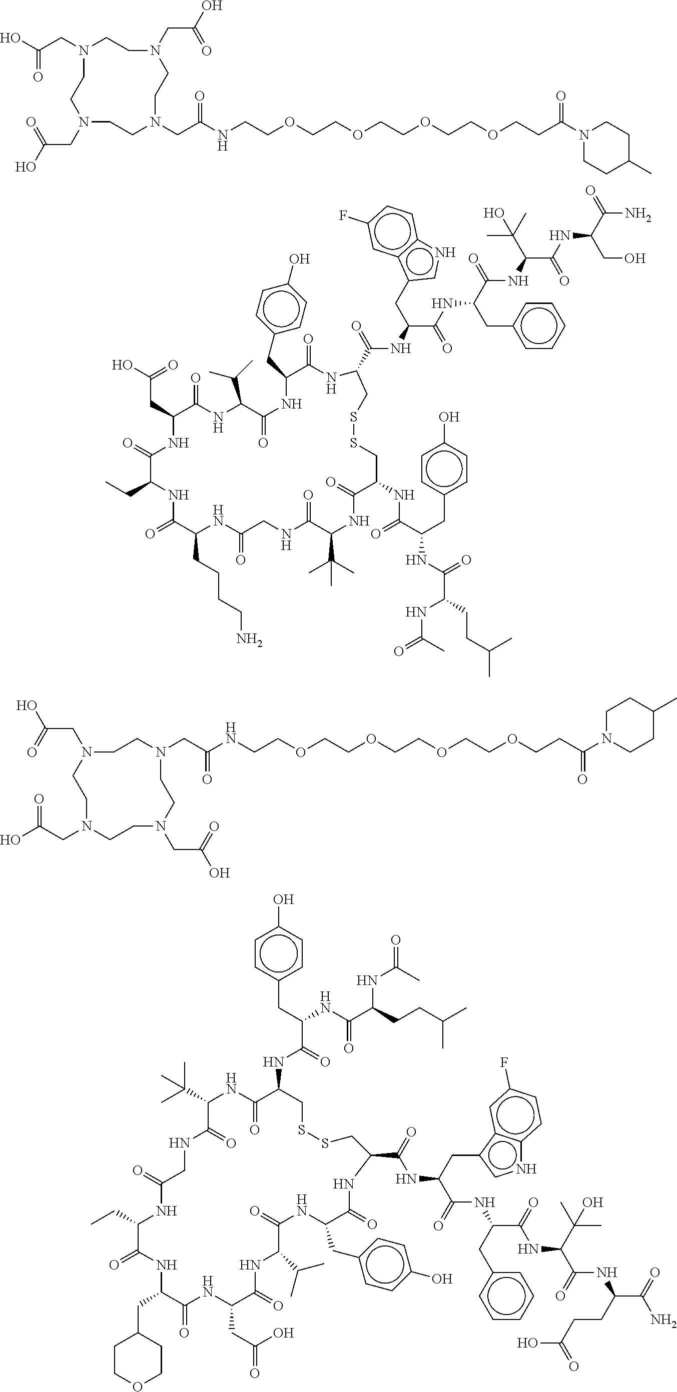

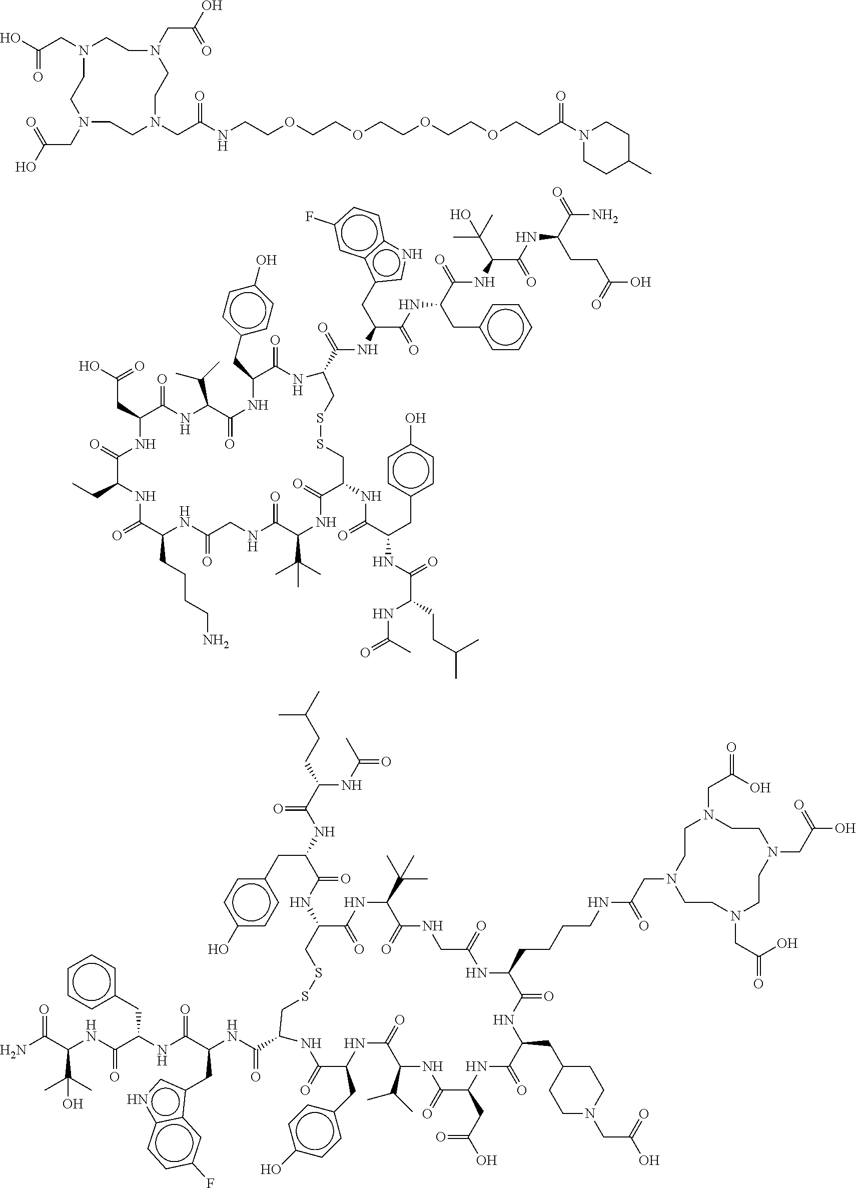

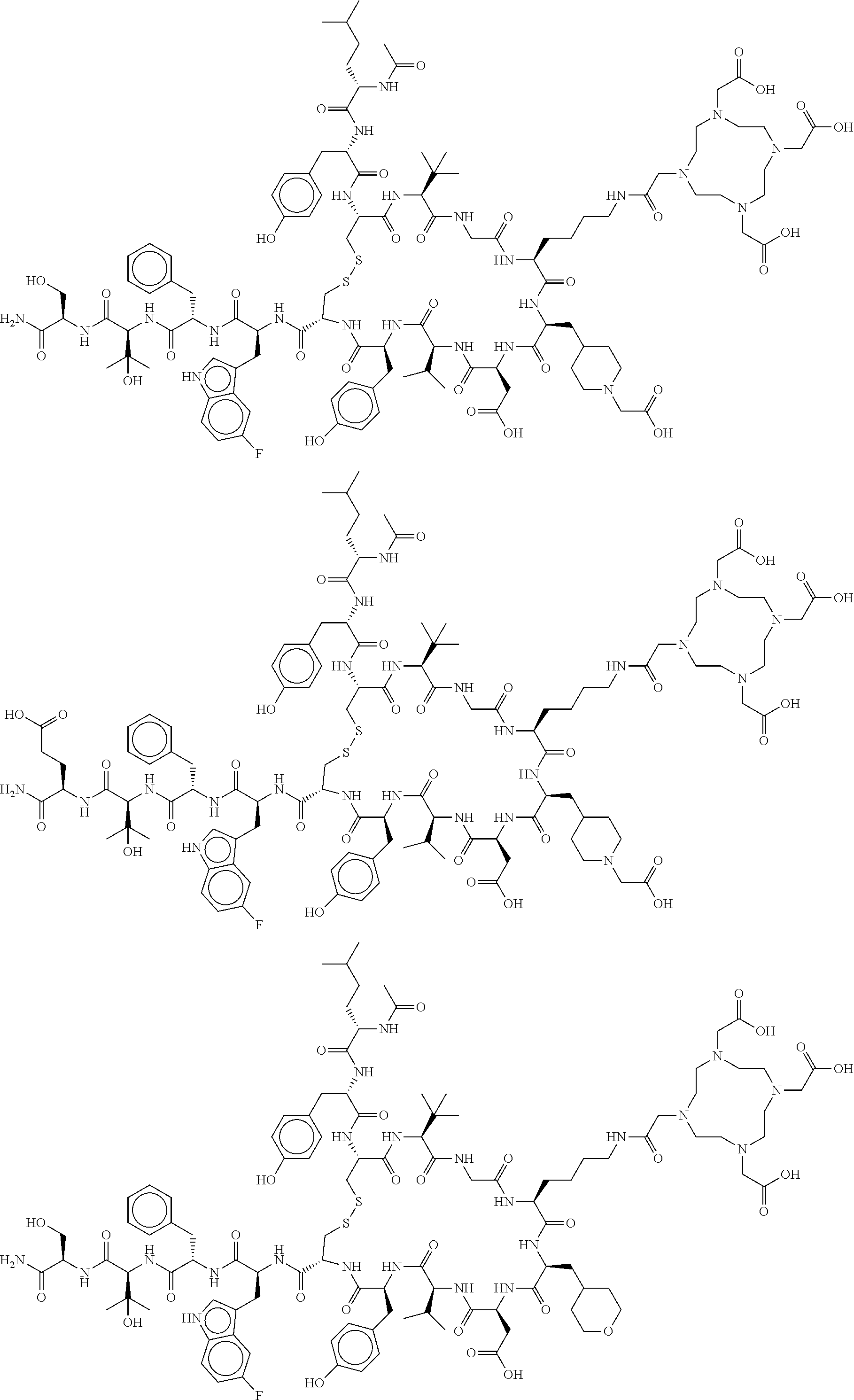

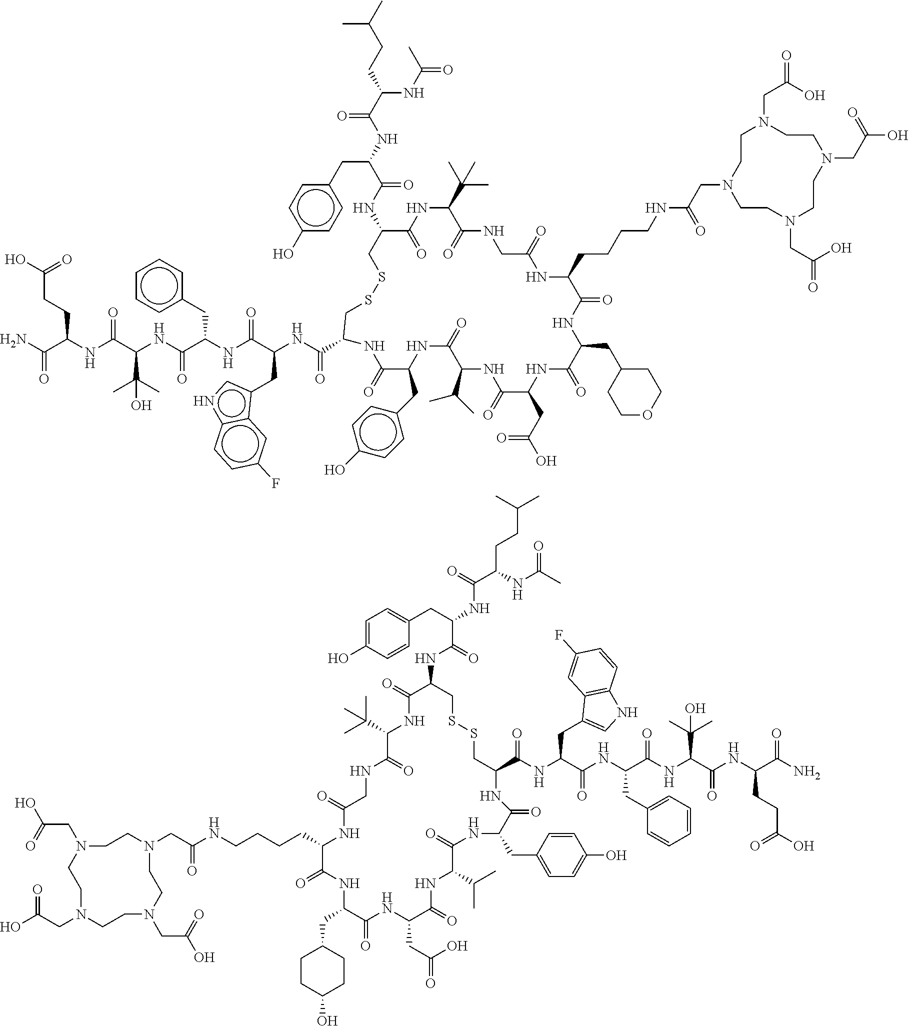

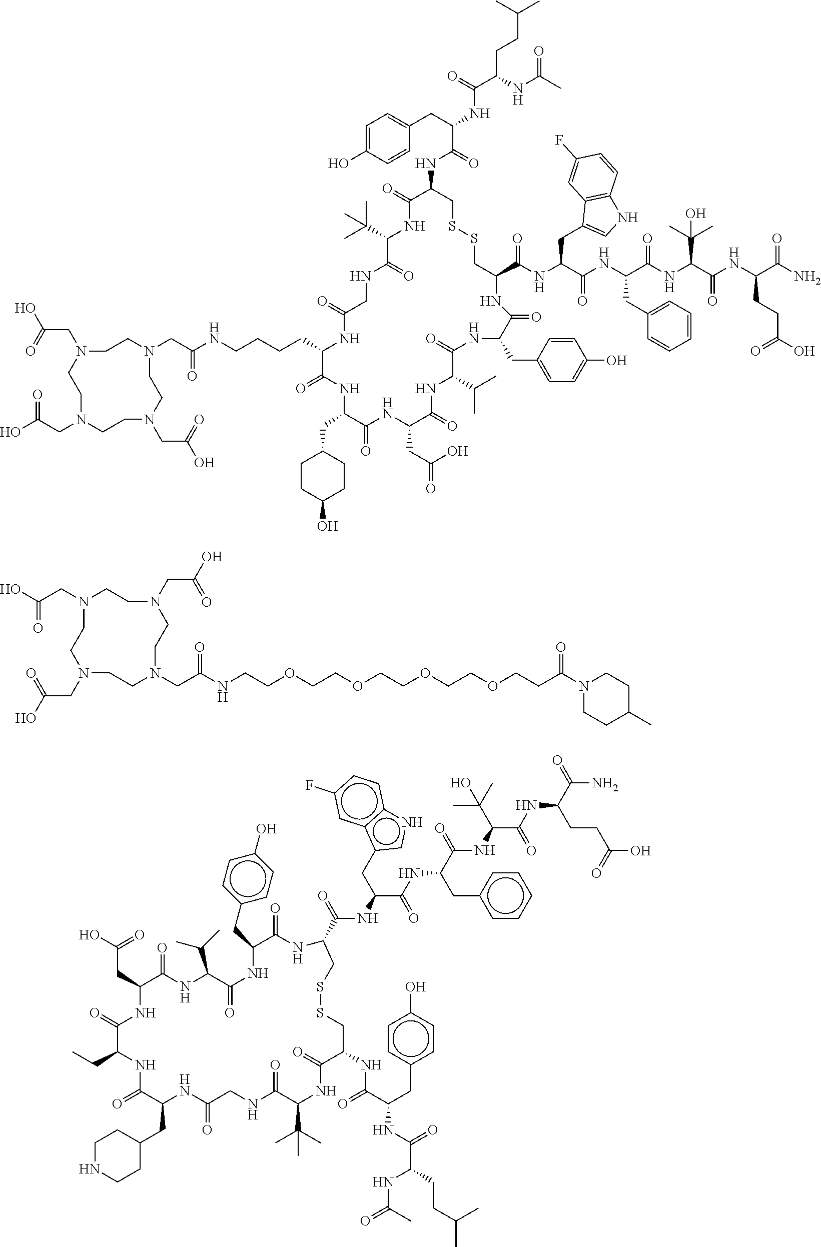

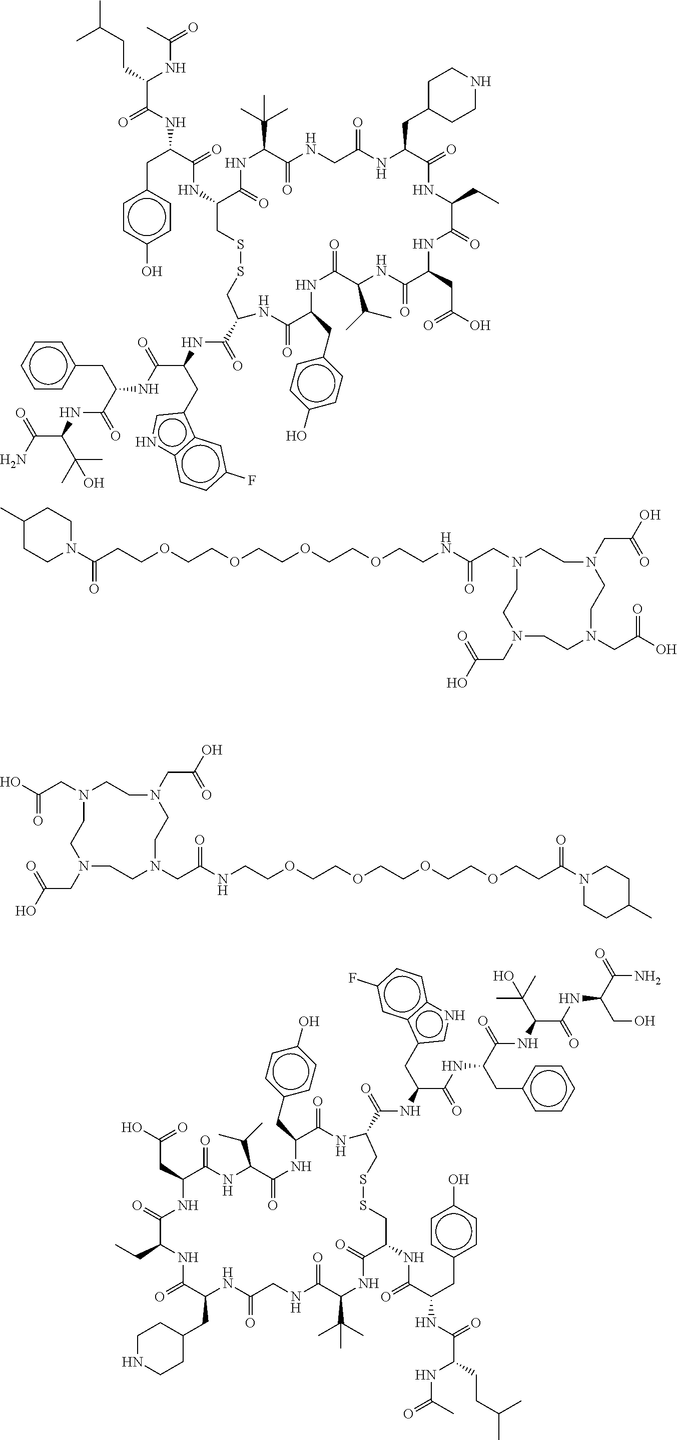

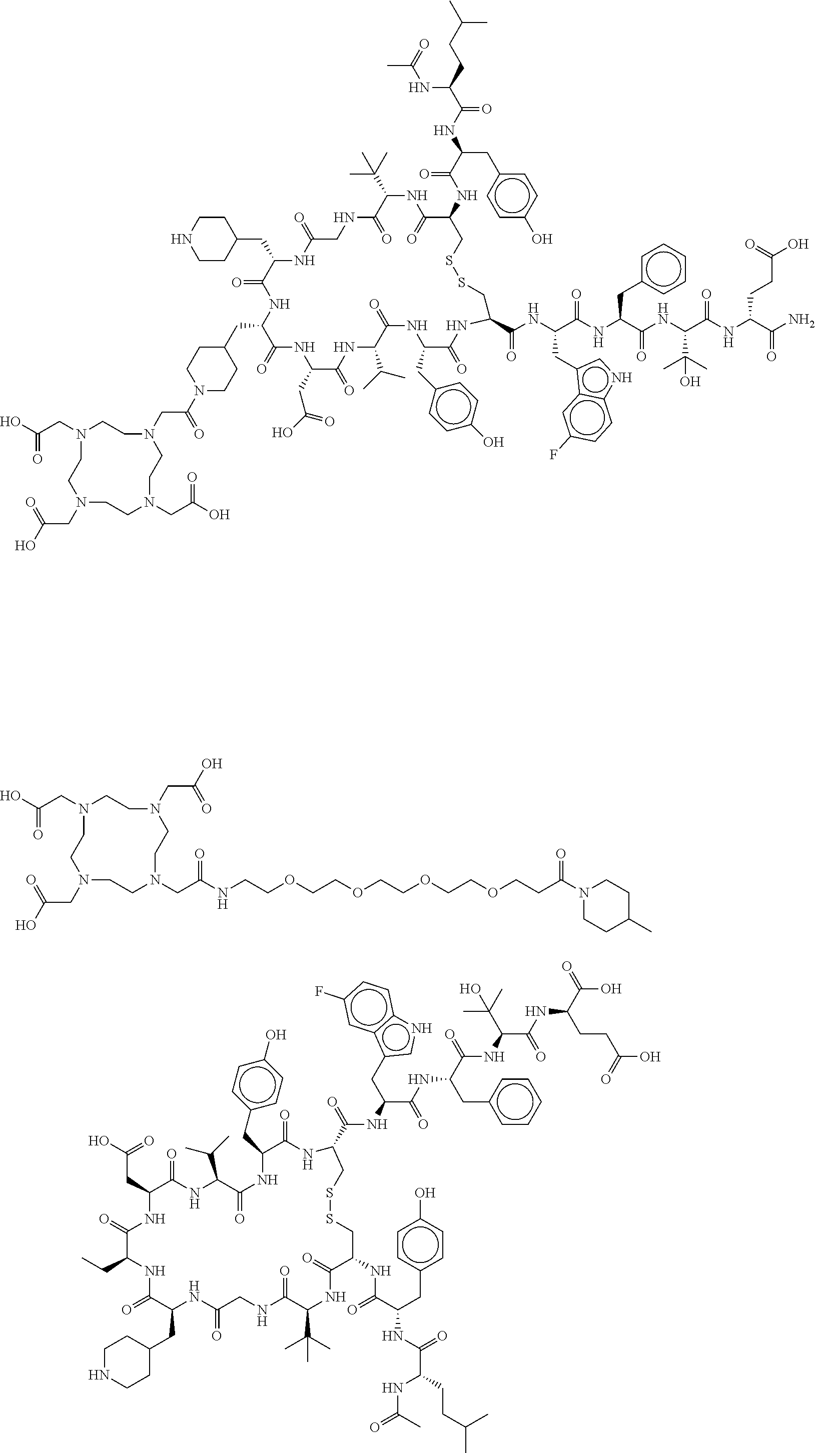

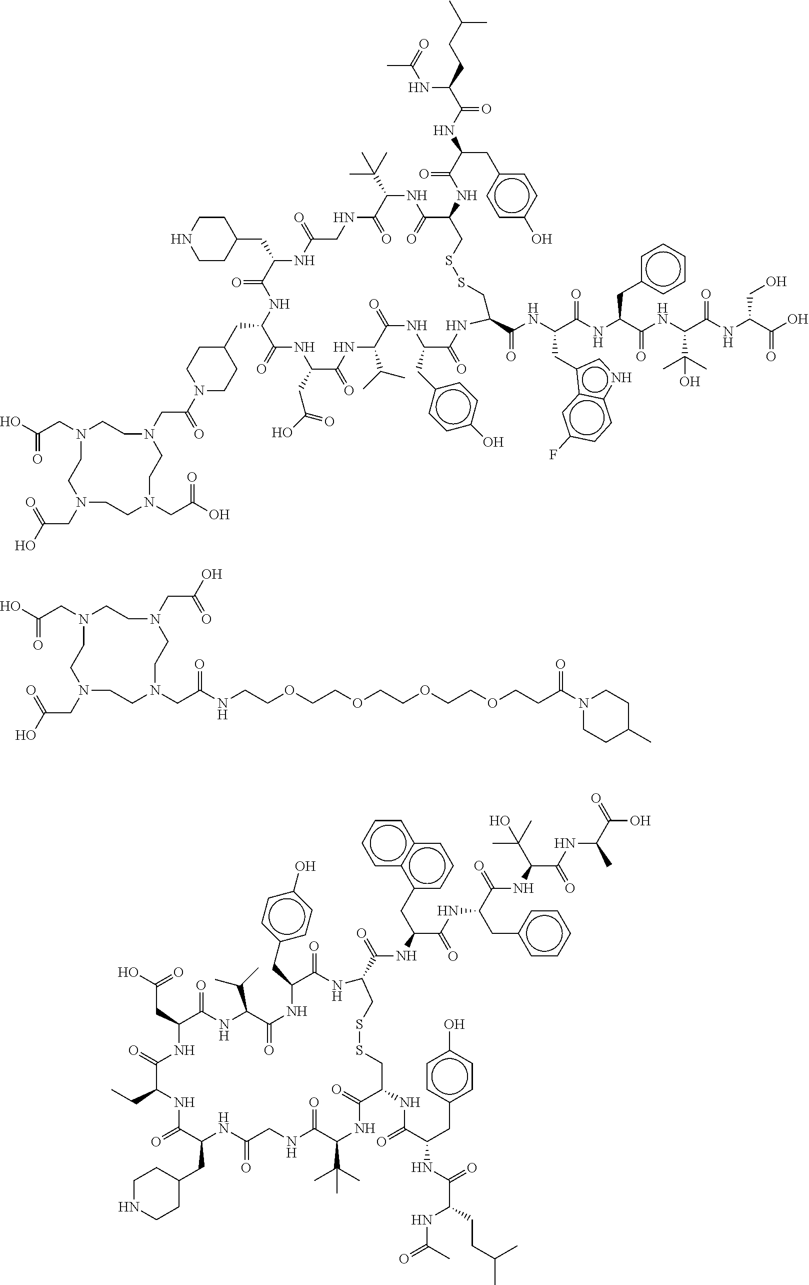

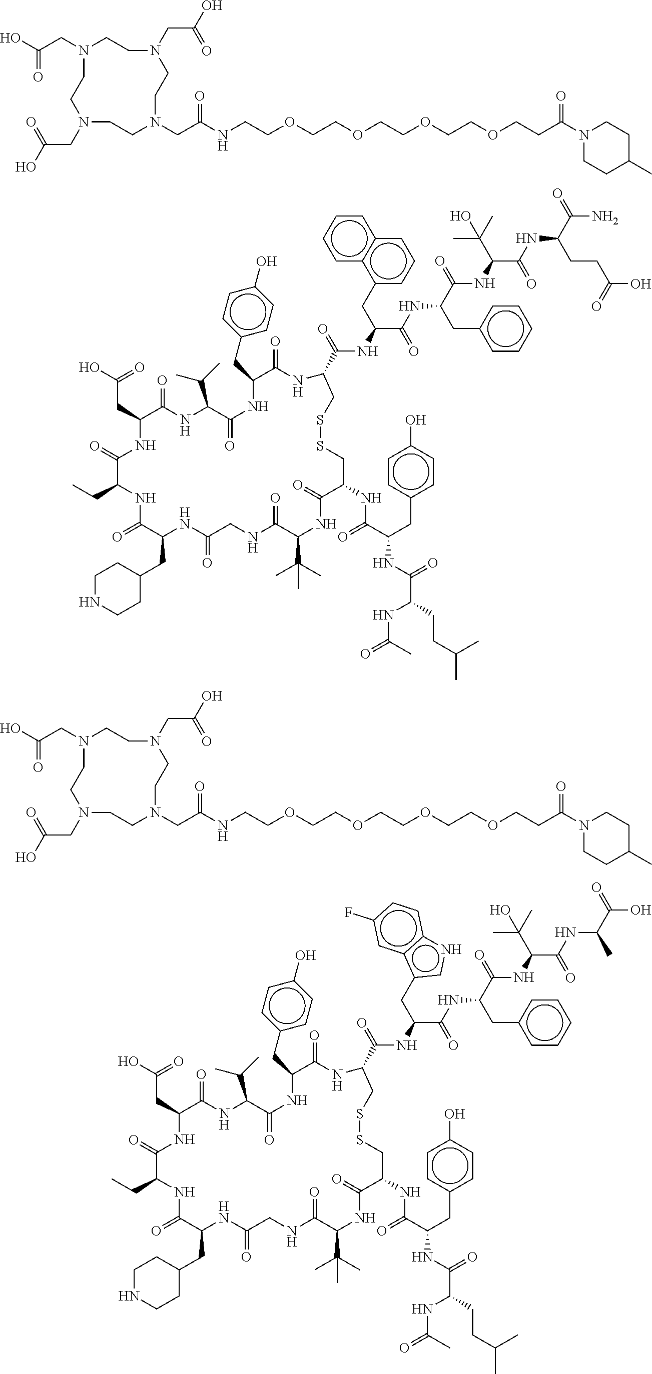

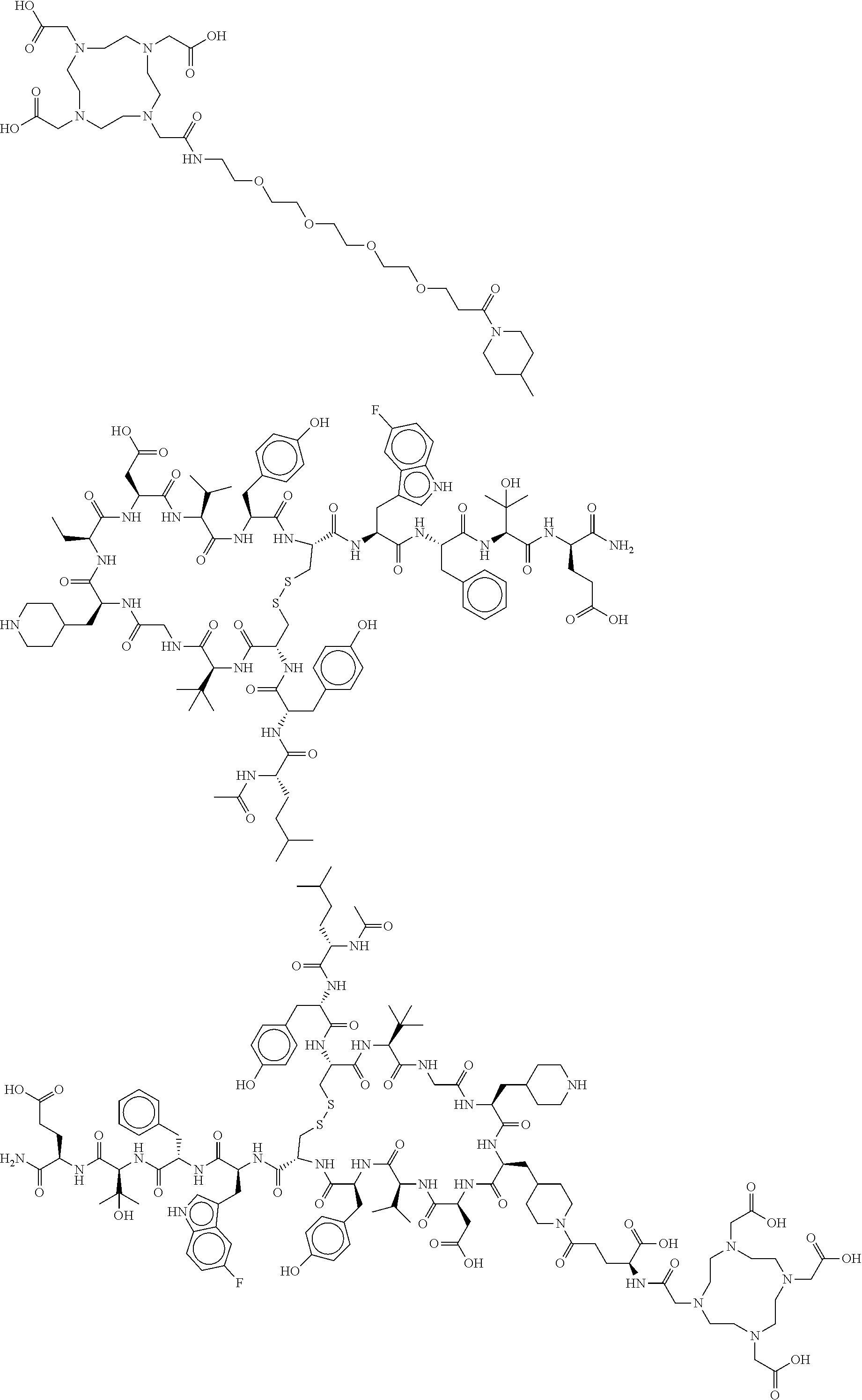

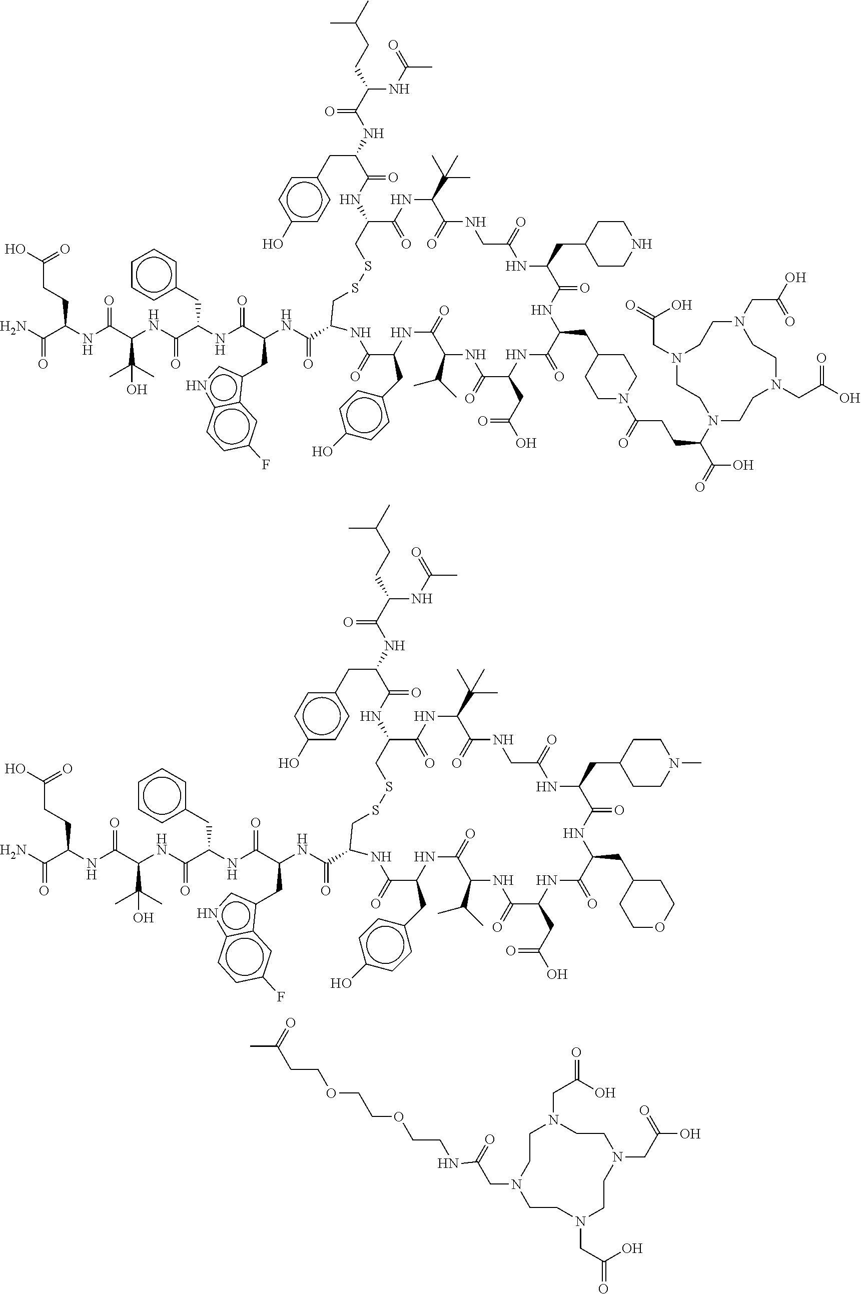

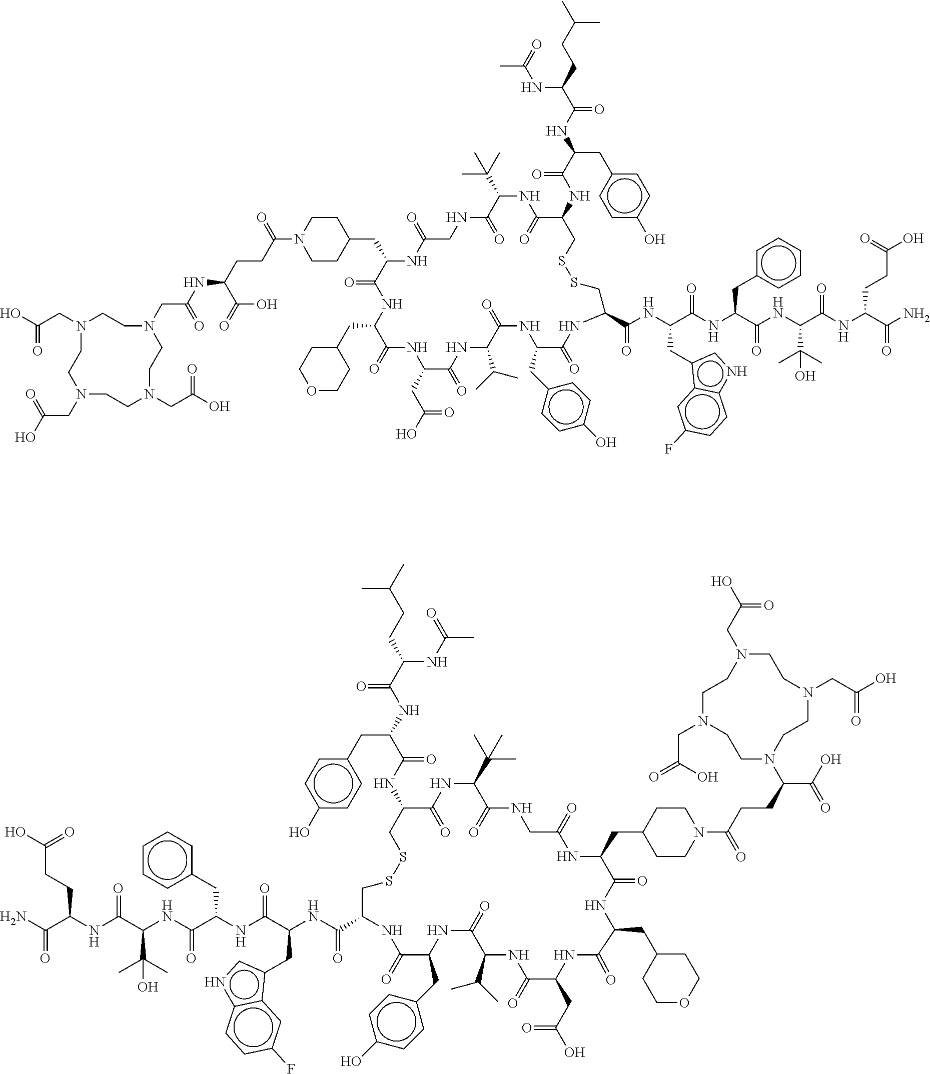

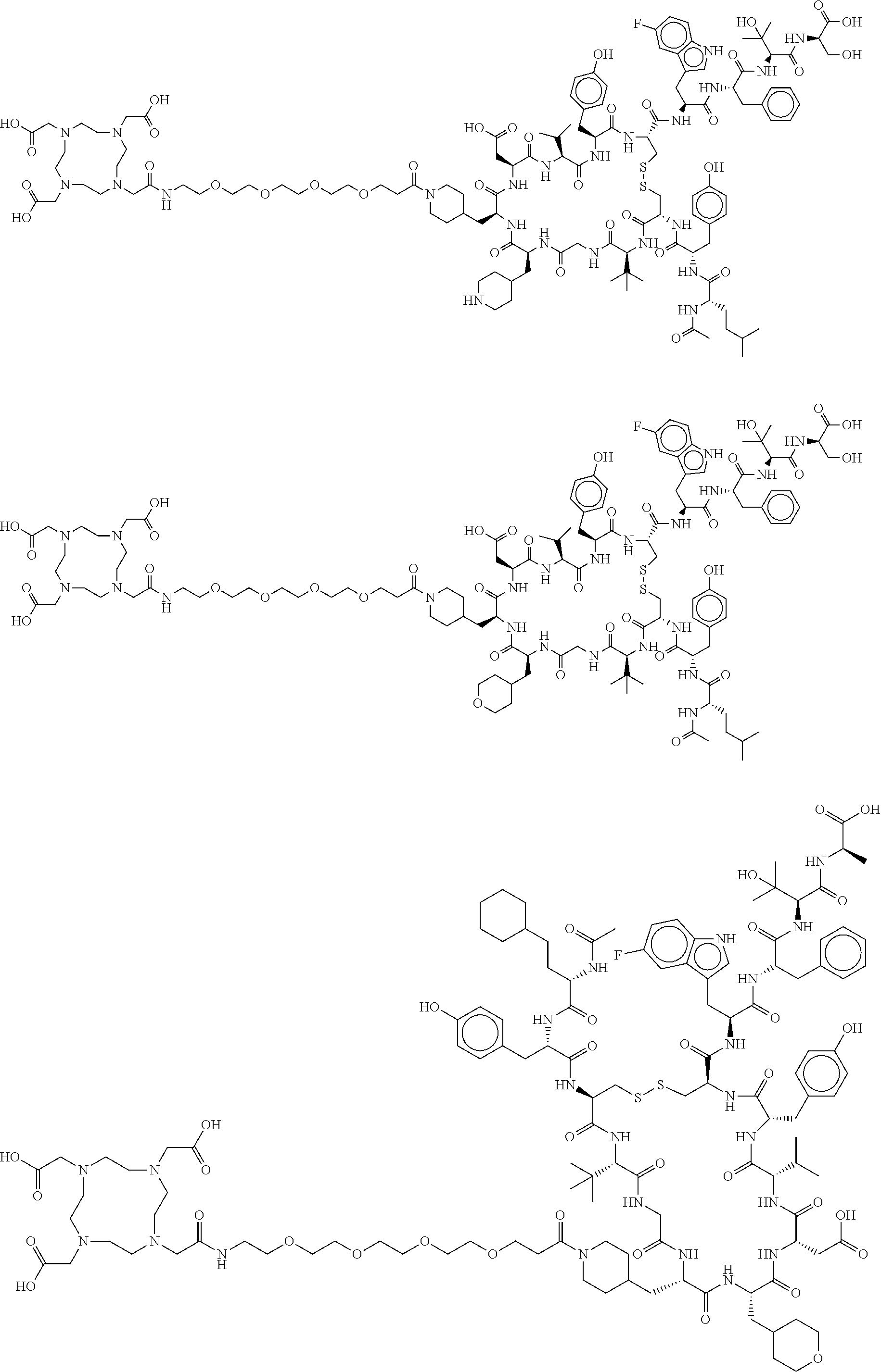

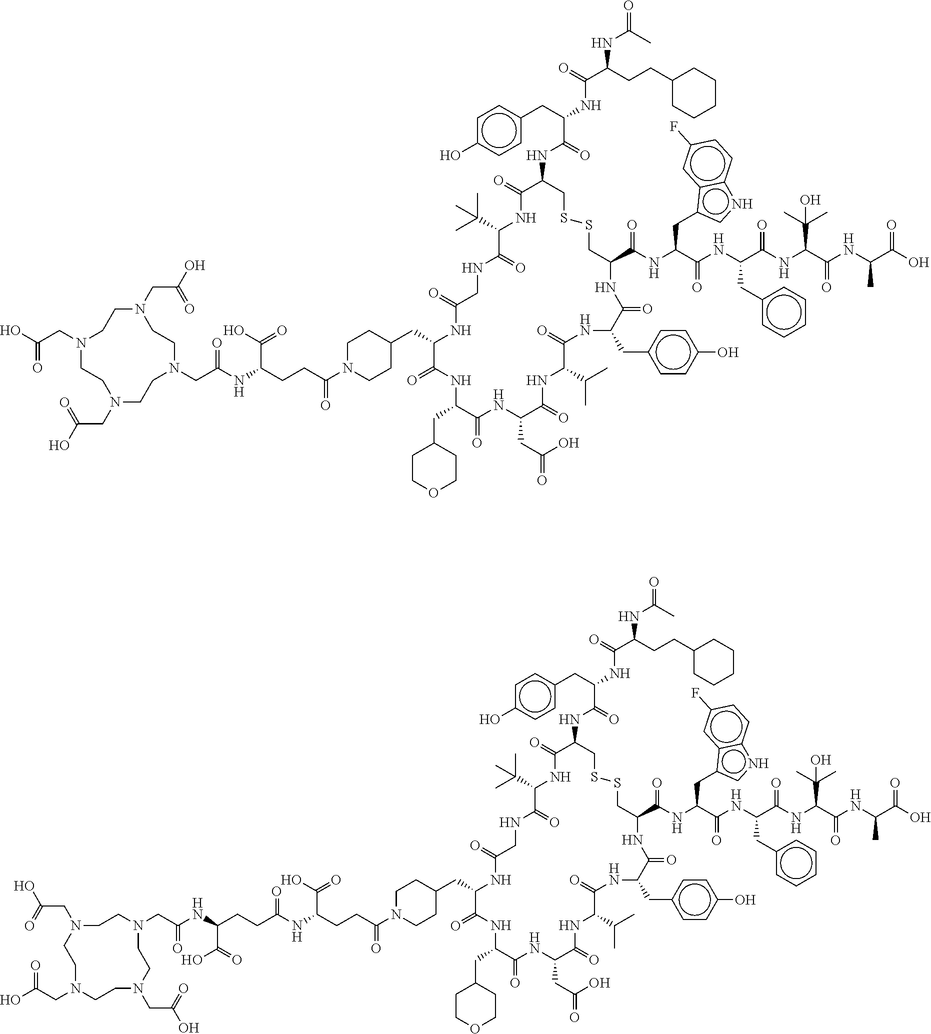

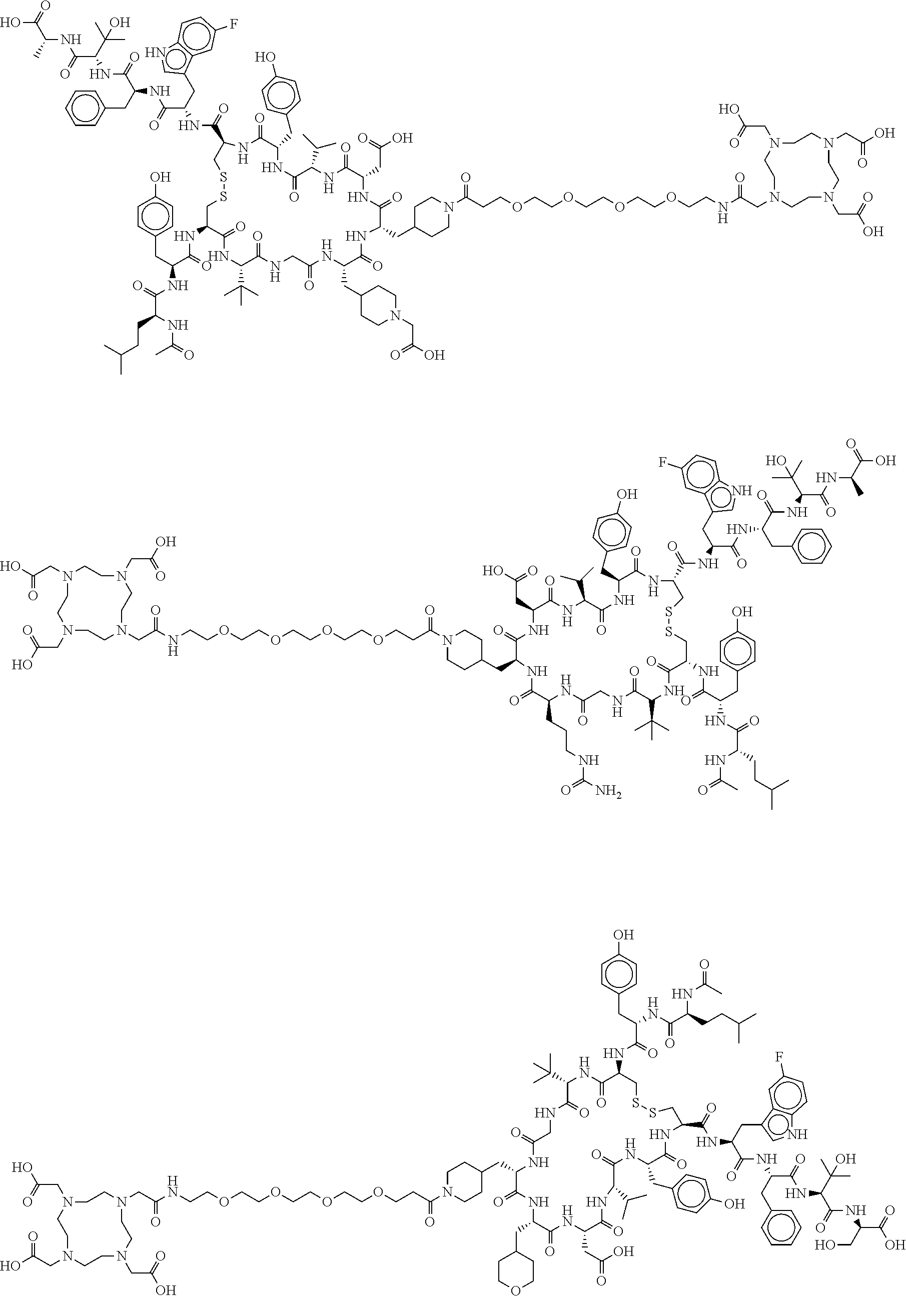

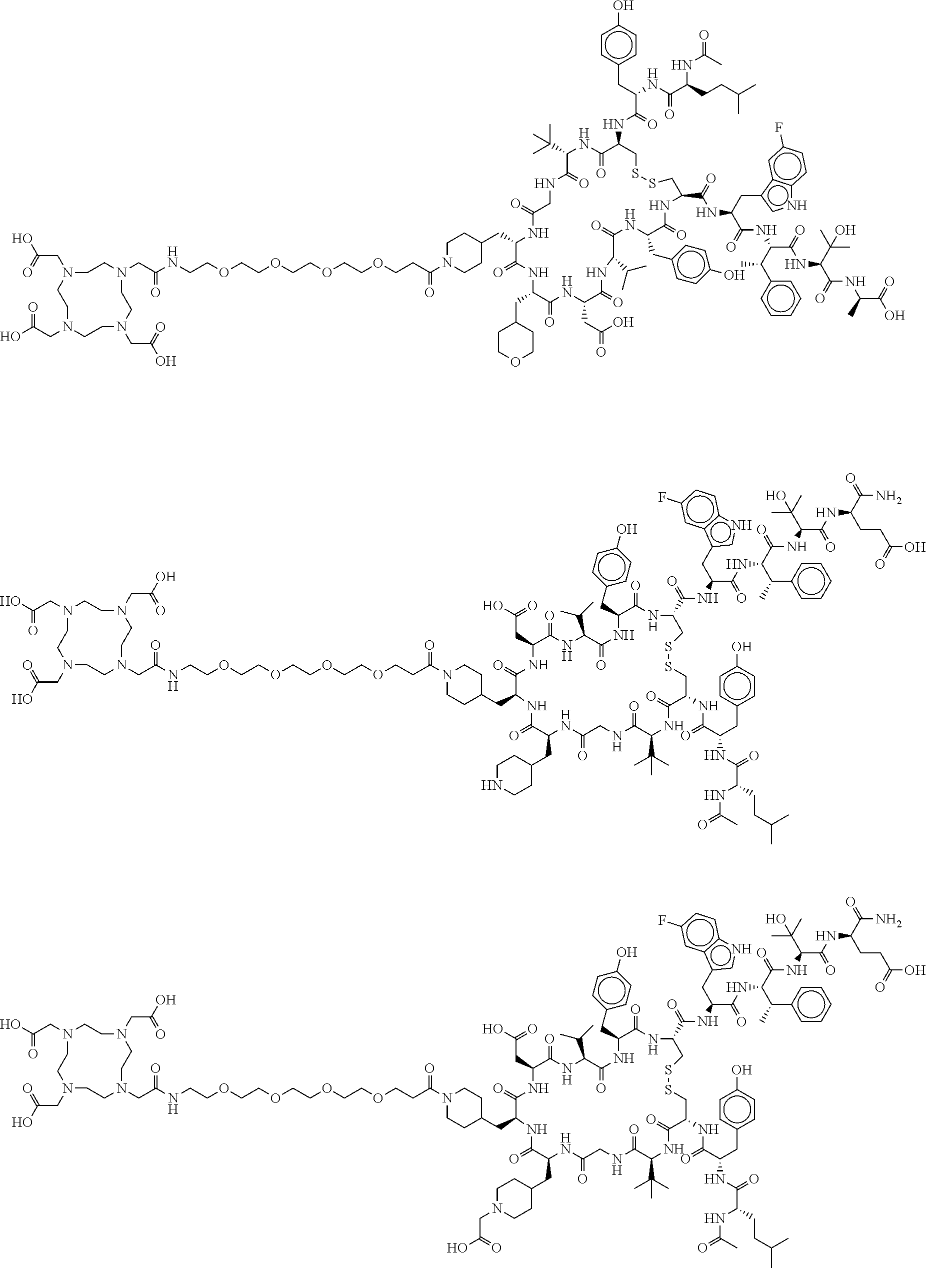

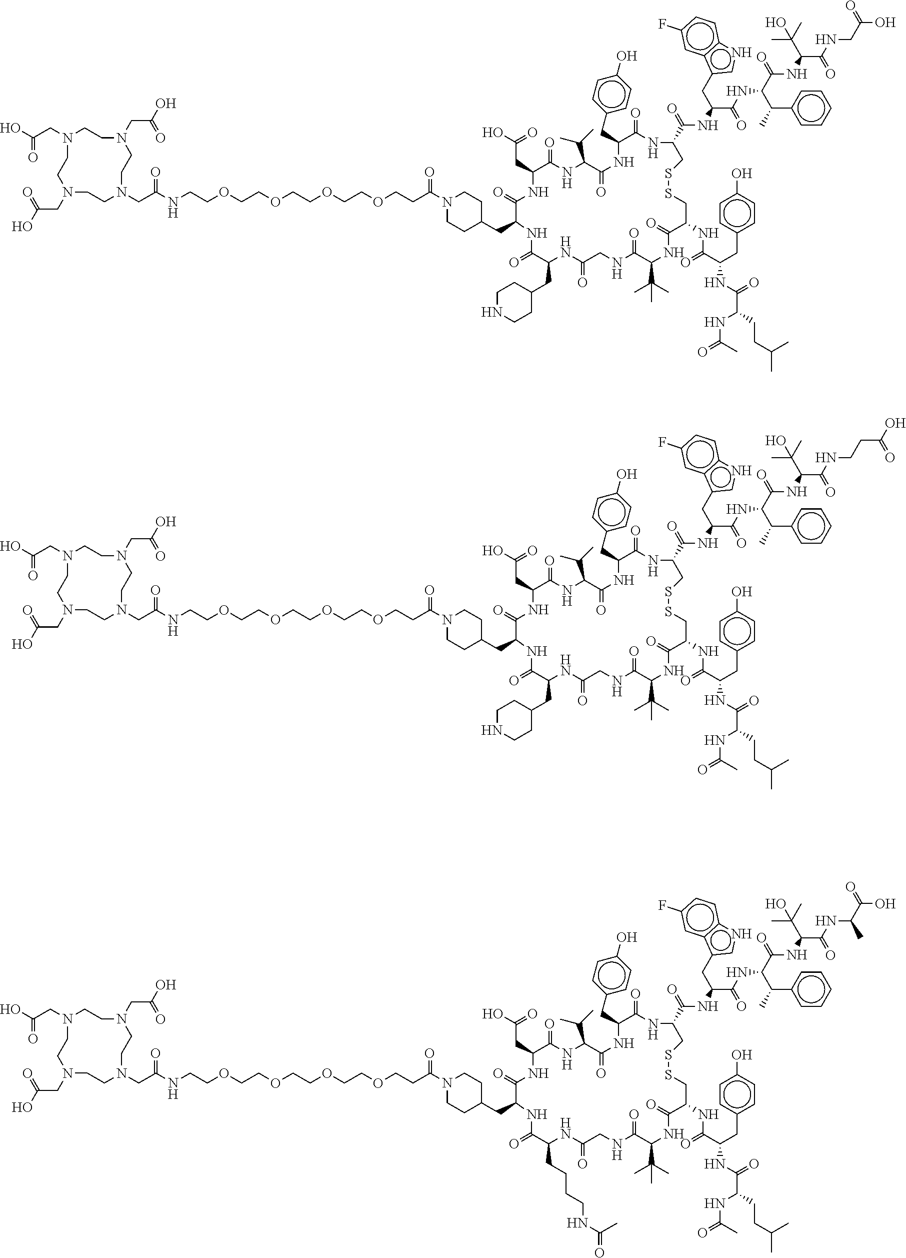

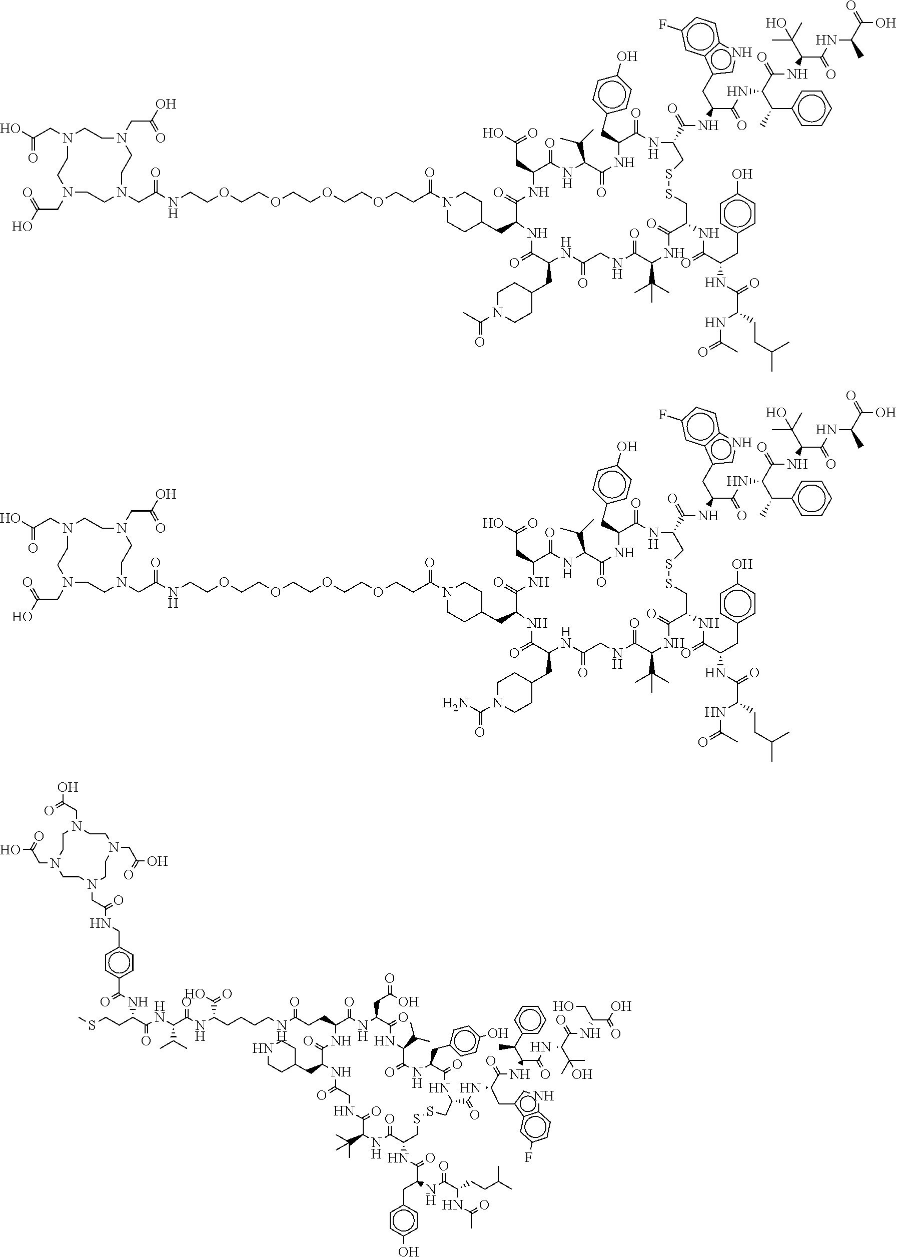

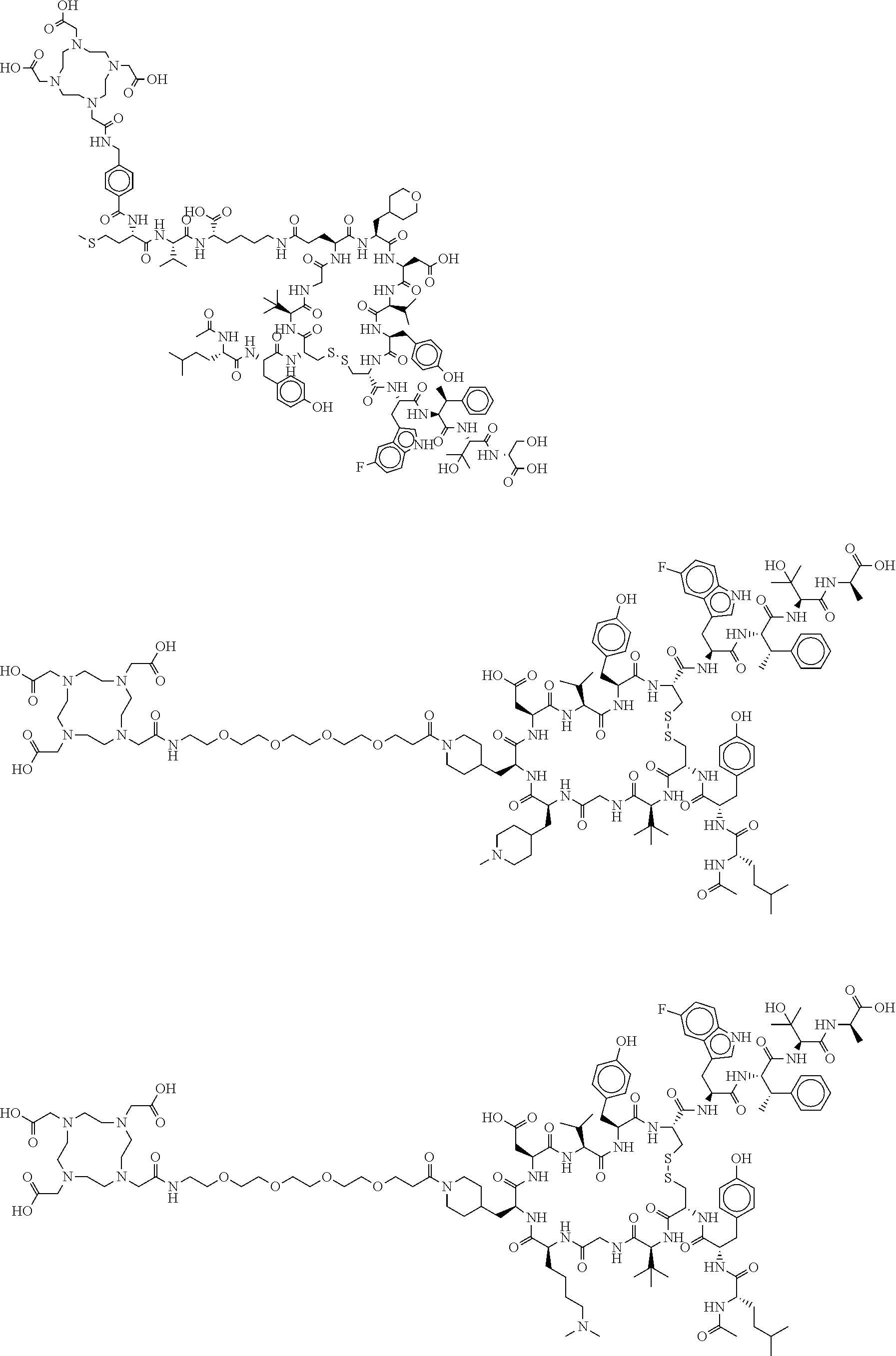

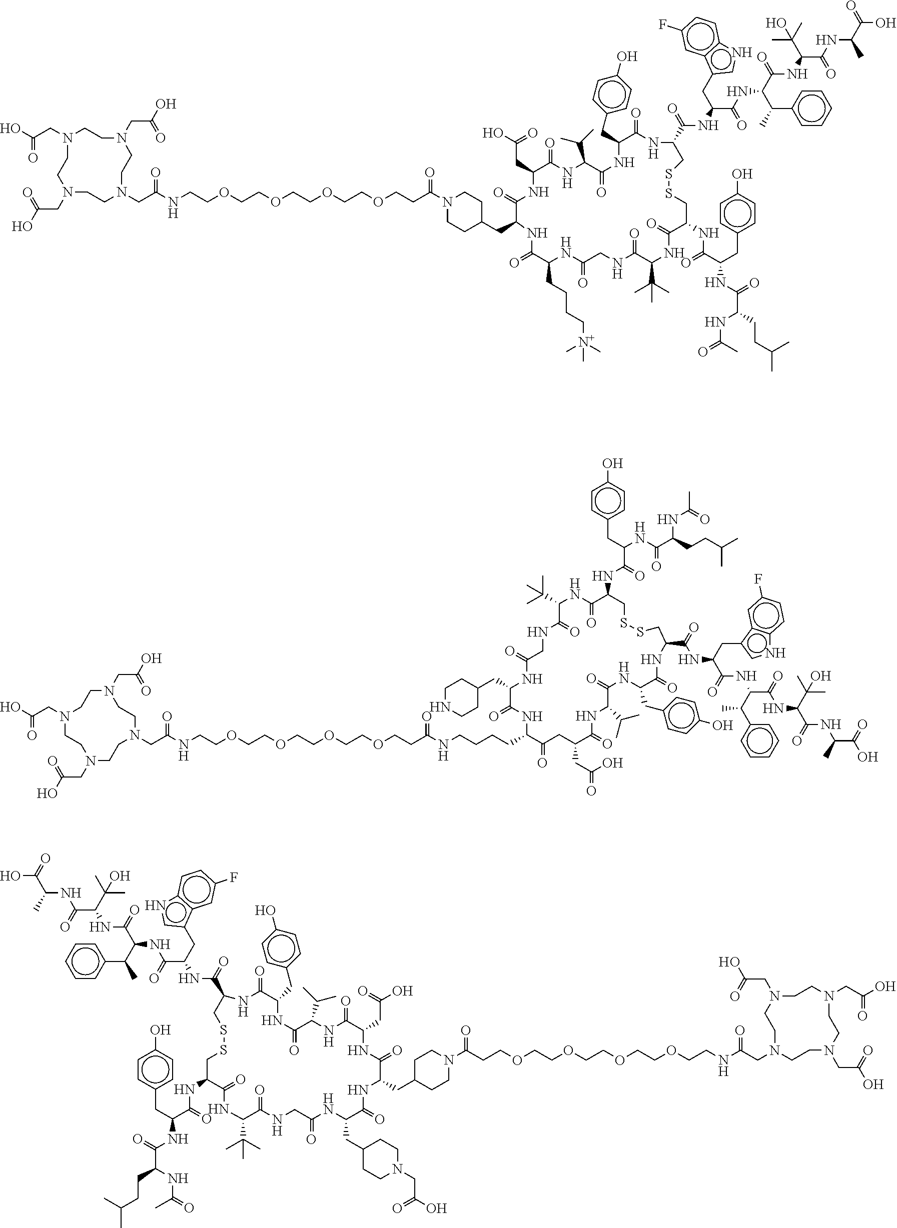

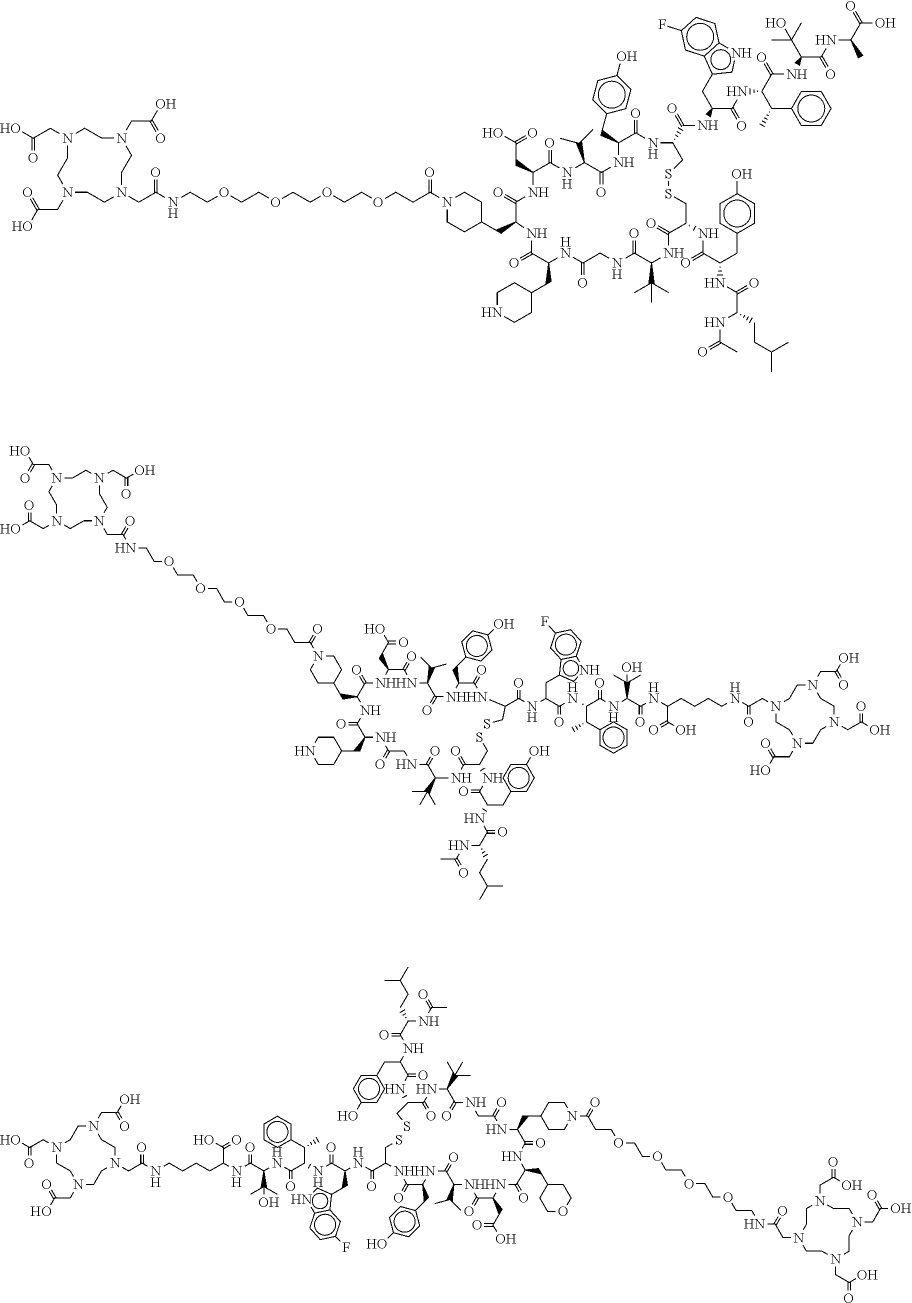

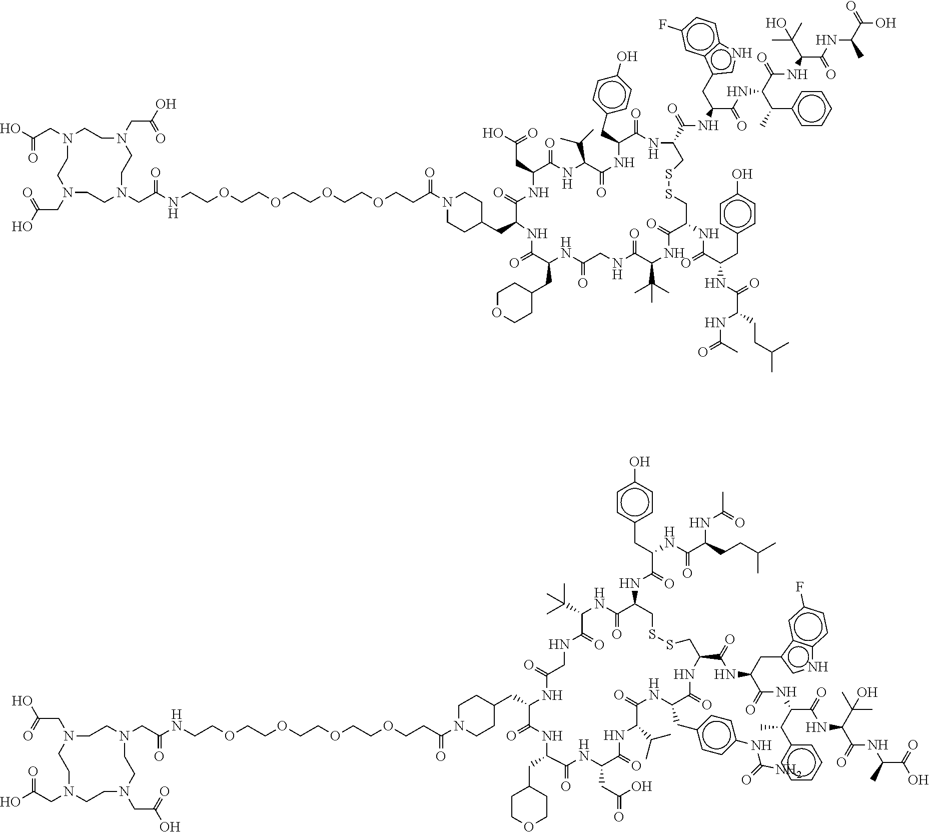

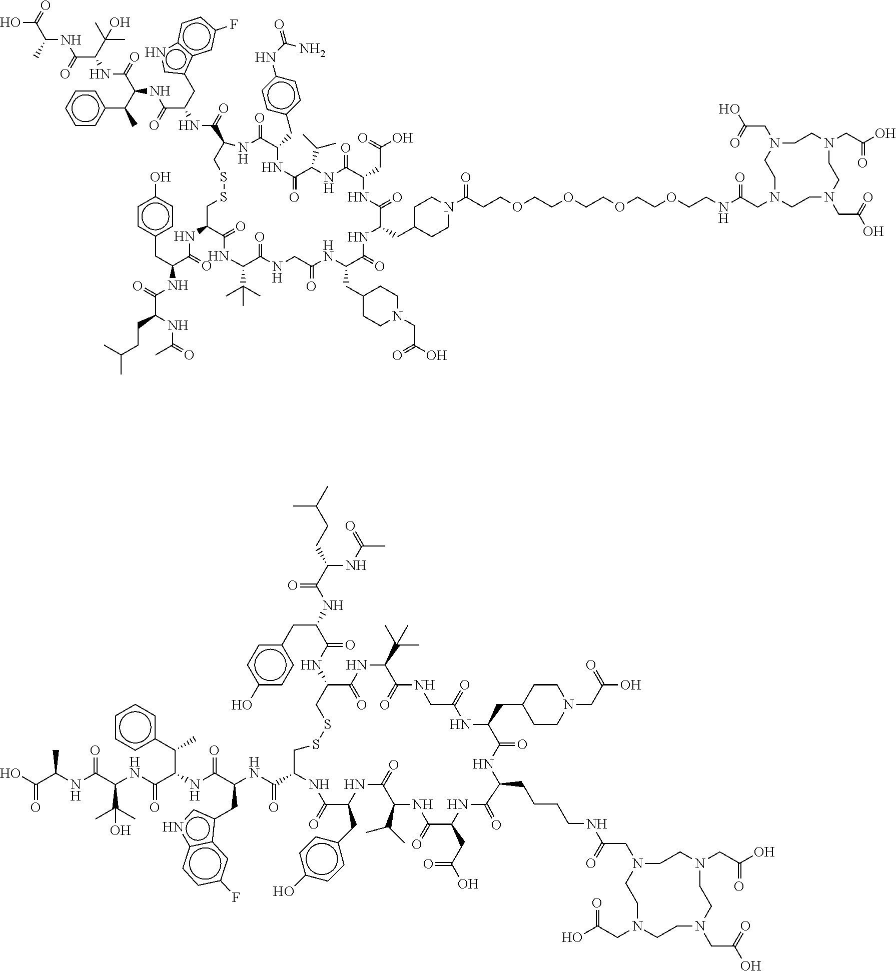

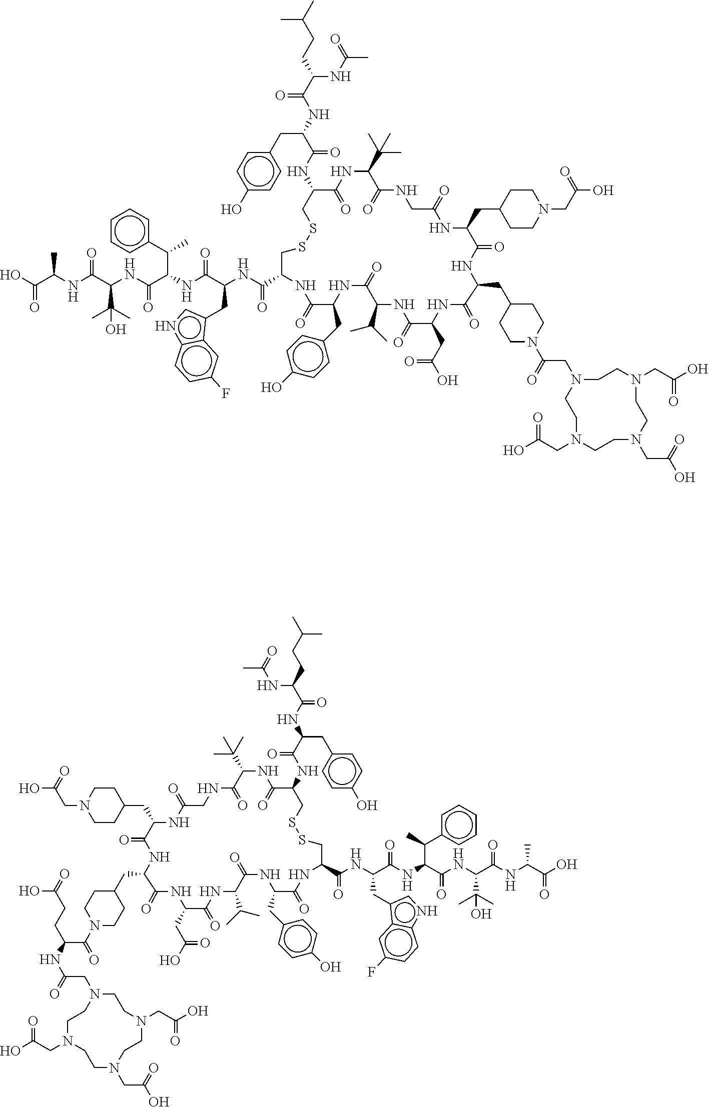

37. The cyclic peptide of claim 1, wherein the cyclic peptide is further complexed with a radionuclide.

38-40. (canceled)

41. A pharmaceutical composition comprising the cyclic peptide of claim 1, or a pharmaceutically acceptable salt thereof, and a pharmaceutically acceptable carrier.

42. A method of treating cancer in a subject in need thereof comprising administering to the subject the cyclic peptide of claim 1, or a pharmaceutically acceptable salt thereof.

43. The method of claim 42, wherein the cancer is a B7H3-mediated cancer.

44. The method of claim 42, wherein the cancer is lung cancer, urothelial cancer, melanoma, squamous cell carcinoma, endometrial cancer, breast cancer, acute myeloid leukemia (AML), gastric cancer, colorectal cancer, prostate cancer, glioma, ovarian cancer, liver cancer, cervical cancer, esophageal cancer, and head and neck cancer.

45-48. (canceled)

49-61. (canceled)

62. A method of inhibiting the activity of B7-H3 isoform 4lg (4lg-B7-H3) on a cell, comprising contacting a cell with a cyclic peptide having binding specificity for 4lg-B7-H3, thereby inhibiting the activity of 4lg-B7-H3 on the cell.

63-69. (canceled)

70. A method of imaging a subject, comprising administering to the subject a cyclic peptide according to claim 1, or a pharmaceutical composition thereof, and obtaining an image of the subject.


SEQUENCE LISTING (574 sequences)

SEQ ID NO: 1 - protein, 13 aa, synthetic construct
  SITE 13: note = Amidated residue
  SITE 3..11: note = Intrachain disulfide bridge
MYCVGKYDVYCWF

SEQ ID NO: 2 - protein, 14 aa, synthetic construct
  SITE 14: note = Amidated D-Lysine modified with 1,4,7,10-tetra-azacylcododecane-N,N,N,N-tetraacetic acid via the side chain
  SITE 3..11: note = Intrachain disulfide bridge
MYCVGKYDVYCWFK

SEQ ID NO: 3 - protein, 13 aa, synthetic construct
  SITE 13: note = Amidated residue
  SITE 3..11: note = Intrachain disulfide bridge
MYCVGKYDVYCWF

SEQ ID NO: 4 - protein, 14 aa, synthetic construct
  SITE 1: note = Norleucine
  SITE 14: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFK

SEQ ID NO: 5 - protein, 13 aa, synthetic construct
  SITE 1: note = Norleucine
  SITE 13: note = Amidated residue
  SITE 3..11: note = Intrachain disulfide bridge
XYCVGKYDVYCWF

SEQ ID NO: 6 - protein, 13 aa, synthetic construct
  SITE 3..11: note = Intrachain disulfide bridge
MYCVGKYDVYCWF

SEQ ID NO: 7 - protein, 13 aa, synthetic construct
  SITE 1: note = Norleucine
  SITE 13: note = Amidated residue
  SITE 3..11: note = Intrachain disulfide bridge
XYCVGKYDVYCWF

SEQ ID NO: 8 - protein, 13 aa, synthetic construct
  SITE 2: note = D-Alanine
  SITE 13: note = Amidated residue
  SITE 3..11: note = Intrachain disulfide bridge
MACVGKYDVYCWF

SEQ ID NO: 9 - protein, 13 aa, synthetic construct
  SITE 5: note = D-Alanine
  SITE 13: note = Amidated residue
  SITE 3..11: note = Intrachain disulfide bridge
MYCVAKYDVYCWF

SEQ ID NO: 10 - protein, 13 aa, synthetic construct
  SITE 13: note = Amidated residue
  SITE 3..11: note = Intrachain disulfide bridge
MYCVGKADVYCWF

SEQ ID NO: 11 - protein, 13 aa, synthetic construct
  SITE 7: note = D-Alanine
  SITE 13: note = Amidated residue
  SITE 3..11: note = Intrachain disulfide bridge
MYCVGKADVYCWF

SEQ ID NO: 12 - protein, 13 aa, synthetic construct
  SITE 13: note = Amidated residue
  SITE 3..11: note = Intrachain disulfide bridge
MYCVGKYAVYCWF

SEQ ID NO: 13 - protein, 13 aa, synthetic construct
  SITE 8: note = D-Alanine
  SITE 13: note = Amidated residue
  SITE 3..11: note = Intrachain disulfide bridge
MYCVGKYAVYCWF

SEQ ID NO: 14 - protein, 13 aa, synthetic construct
  SITE 13: note = Amidated residue
  SITE 3..11: note = Intrachain disulfide bridge
MYCVGKYDAYCWF

SEQ ID NO: 15 - protein, 13 aa, synthetic construct
  SITE 9: note = D-Alanine
  SITE 13: note = Amidated residue
  SITE 3..11: note = Intrachain disulfide bridge
MYCVGKYDAYCWF

SEQ ID NO: 16 - protein, 13 aa, synthetic construct
  SITE 13: note = Amidated residue
  SITE 3..11: note = Intrachain disulfide bridge
MYCVGKYDVACWF

SEQ ID NO: 17 - protein, 13 aa, synthetic construct
  SITE 13: note = Amidated residue
  SITE 3..11: note = Intrachain disulfide bridge
MYCVGKYDVYCAF

SEQ ID NO: 18 - protein, 13 aa, synthetic construct
  SITE 1: note = Homoleucine
  SITE 13: note = Amidated residue
  SITE 3..11: note = Intrachain disulfide bridge
XYCVGKYDVYCWF

SEQ ID NO: 19 - protein, 13 aa, synthetic construct
  SITE 13: note = Amidated residue
  SITE 3..11: note = Intrachain disulfide bridge
LYCVGKYDVYCWF

SEQ ID NO: 20 - protein, 13 aa, synthetic construct
  SITE 1: note = Norleucine
  SITE 2: note = 3-fluorotyrosine
  SITE 13: note = Amidated residue
  SITE 3..11: note = Intrachain disulfide bridge
XXCVGKYDVYCWF

SEQ ID NO: 21 - protein, 13 aa, synthetic construct
  SITE 1: note = Norleucine
  SITE 2: note = 3-chlorotyrosine
  SITE 13: note = Amidated residue
  SITE 3..11: note = Intrachain disulfide bridge
XXCVGKYDVYCWF

SEQ ID NO: 22 - protein, 13 aa, synthetic construct
  SITE 1: note = Norleucine
  SITE 2: note = 4-carboxyphenylalanine
  SITE 13: note = Amidated residue
  SITE 3..11: note = Intrachain disulfide bridge
XXCVGKYDVYCWF

SEQ ID NO: 23 - protein, 13 aa, synthetic construct
  SITE 1: note = Norleucine
  SITE 2: note = 2-amino-3-(pyridin-4-yl)propanoic acid
  SITE 13: note = Amidated residue
  SITE 3..11: note = Intrachain disulfide bridge
XXCVGKYDVYCWF

SEQ ID NO: 24 - protein, 13 aa, synthetic construct
  SITE 1: note = Norleucine
  SITE 7: note = 4-carboxyphenylalanine
  SITE 13: note = Amidated residue
  SITE 3..11: note = Intrachain disulfide bridge
XYCVGKXDVYCWF

SEQ ID NO: 25 - protein, 13 aa, synthetic construct
  SITE 1: note = Norleucine
  SITE 7: note = 2-amino-3-(pyridin-4-yl)propanoic acid
  SITE 13: note = Amidated residue
  SITE 3..11: note = Intrachain disulfide bridge
XYCVGKXDVYCWF

SEQ ID NO: 26 - protein, 13 aa, synthetic construct
  SITE 1: note = Norleucine
  SITE 5: note = Sarcosine
  SITE 13: note = Amidated residue
  SITE 3..11: note = Intrachain disulfide bridge
XYCVXKYDVYCWF

SEQ ID NO: 27 - protein, 15 aa, synthetic construct
  SITE 1: note = Norleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFTK

SEQ ID NO: 28 - protein, 15 aa, synthetic construct
  SITE 1: note = Norleucine
  SITE 15: note = D-Lysine modified with 1,4,7,10-tetra-azacylcododecane-N,N,N,N-tetraacetic acid
  SITE 3..11: note = Intrachain disulfide bridge
XYCVGKYDVYCWFTK

SEQ ID NO: 29 - protein, 15 aa, synthetic construct
  SITE 1: note = Norleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFEK

SEQ ID NO: 30 - protein, 16 aa, synthetic construct
  SITE 1: note = Norleucine
  SITE 14: note = Gamma-glutamic acid
  SITE 15: note = Gamma-glutamic acid
  SITE 16: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFEEK

SEQ ID NO: 31 - protein, 15 aa, synthetic construct
  SITE 1: note = Norleucine
  SITE 14: note = Gamma-glutamic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFEK

SEQ ID NO: 32 - protein, 14 aa, synthetic construct
  SITE 1: note = Norleucine
  SITE 14: note = Amidated residue
  SITE 3..11: note = Intrachain disulfide bridge
XYCVGKYDVYCWFT

SEQ ID NO: 33 - protein, 14 aa, synthetic construct
  SITE 1: note = Norleucine
  SITE 3..11: note = Intrachain disulfide bridge
XYCVGKYDVYCWFT

SEQ ID NO: 34 - protein, 14 aa, synthetic construct
  SITE 1: note = Norleucine
  SITE 14: note = Amidated residue
  SITE 3..11: note = Intrachain disulfide bridge
XYCVGKYDVYCWFE

SEQ ID NO: 35 - protein, 14 aa, synthetic construct
  SITE 1: note = Norleucine
  SITE 14: note = Amidated gamma-glutamic acid
  SITE 3..11: note = Intrachain disulfide bridge
XYCVGKYDVYCWFE

SEQ ID NO: 36 - protein, 15 aa, synthetic construct
  SITE 1: note = Norleucine
  SITE 14: note = Gamma-glutamic acid
  SITE 15: note = Amidated gamma-glutamic acid
  SITE 3..11: note = Intrachain disulfide bridge
XYCVGKYDVYCWFEE

SEQ ID NO: 37 - protein, 13 aa, synthetic construct
  SITE 13: note = Amidated residue
  SITE 3..11: note = Intrachain disulfide bridge
AYCVGKYDVYCWF

SEQ ID NO: 38 - protein, 13 aa, synthetic construct
  SITE 1: note = D-Alanine
  SITE 13: note = Amidated residue
  SITE 3..11: note = Intrachain disulfide bridge
AYCVGKYDVYCWF

SEQ ID NO: 39 - protein, 13 aa, synthetic construct
  SITE 4: note = D-Alanine
  SITE 13: note = Amidated residue
  SITE 3..11: note = Intrachain disulfide bridge
MYCAGKYDVYCWF

SEQ ID NO: 40 - protein, 13 aa, synthetic construct
  SITE 5: note = Sarcosine
  SITE 13: note = Amidated residue
  SITE 3..11: note = Intrachain disulfide bridge
MYCVXKYDVYCWF

SEQ ID NO: 41 - protein, 13 aa, synthetic construct
  SITE 13: note = Amidated residue
  SITE 3..11: note = Intrachain disulfide bridge
MYCVAKYDVYCWF

SEQ ID NO: 42 - protein, 13 aa, synthetic construct
  SITE 13: note = Amidated residue
  SITE 3..11: note = Intrachain disulfide bridge
MYCVGAYDVYCWF

SEQ ID NO: 43 - protein, 13 aa, synthetic construct
  SITE 6: note = D-Alanine
  SITE 13: note = Amidated residue
  SITE 3..11: note = Intrachain disulfide bridge
MYCVGAYDVYCWF

SEQ ID NO: 44 - protein, 13 aa, synthetic construct
  SITE 10: note = D-Alanine
  SITE 13: note = Amidated residue
  SITE 3..11: note = Intrachain disulfide bridge
MYCVGKYDVACWF

SEQ ID NO: 45 - protein, 13 aa, synthetic construct
  SITE 12: note = D-Alanine
  SITE 13: note = Amidated residue
  SITE 3..11: note = Intrachain disulfide bridge
MYCVGKYDVYCAF

SEQ ID NO: 46 - protein, 13 aa, synthetic construct
  SITE 13: note = Amidated residue
  SITE 3..11: note = Intrachain disulfide bridge
MYCVGKYDVYCWA

SEQ ID NO: 47 - protein, 13 aa, synthetic construct
  SITE 13: note = Amidated D-Alanine
  SITE 3..11: note = Intrachain disulfide bridge
MYCVGKYDVYCWA

SEQ ID NO: 48 - protein, 13 aa, synthetic construct
  SITE 3: note = D-Cysteine
  SITE 13: note = Amidated residue
  SITE 3..11: note = Intrachain disulfide bridge
MYCVGKYDVYCWF

SEQ ID NO: 49 - protein, 13 aa, synthetic construct
  SITE 11: note = D-Cysteine
  SITE 13: note = Amidated residue
  SITE 3..11: note = Intrachain disulfide bridge
MYCVGKYDVYCWF

SEQ ID NO: 50 - protein, 14 aa, synthetic construct
  SITE 1: note = Norleucine
  SITE 14: note = Amidated residue modified with 1,4,7,10-tetra-azacylcododecane-N,N,N,N-tetraacetic acid via the side chain
  SITE 3..11: note = Intrachain disulfide bridge
XYCVGKYDVYCWFK

SEQ ID NO: 51 - protein, 13 aa, synthetic construct
  SITE 1: note = Norleucine
  SITE 3..11: note = Intrachain disulfide bridge
XYCVSKYDVYCWF

SEQ ID NO: 52 - protein, 13 aa, synthetic construct
  SITE 1: note = Norleucine
  SITE 5: note = N-ethyl glycine
  SITE 13: note = Amidated residue
  SITE 3..11: note = Intrachain disulfide bridge
XYCVXKYDVYCWF

SEQ ID NO: 53 - protein, 13 aa, synthetic construct
  SITE 1: note = Norleucine
  SITE 5: note = 3-((carboxymethyl)amino)propanoic acid
  SITE 13: note = Amidated residue
  SITE 3..11: note = Intrachain disulfide bridge
XYCVXKYDVYCWF

SEQ ID NO: 54 - protein, 15 aa, synthetic construct
  SITE 1: note = Norleucine
  SITE 4: note = (S)-2-amino-2-cyclohexylacetic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCWFTK

SEQ ID NO: 55 - protein, 15 aa, synthetic construct
  SITE 1: note = Norleucine
  SITE 9: note = (S)-2-amino-2-cyclohexylacetic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCWFTK

SEQ ID NO: 56 - protein, 15 aa, synthetic construct
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
LYCVGKYDVYCWFTK

SEQ ID NO: 57 - protein, 15 aa, synthetic construct
  SITE 1: note = Norleucine
  SITE 2: note = 2-amino-3-(pyridin-4-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGKYDVYCWFTK

SEQ ID NO: 58 - protein, 15 aa, synthetic construct
  SITE 1: note = Norleucine
  SITE 7: note = 2-amino-3-(pyridin-4-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCWFTK

SEQ ID NO: 59 - protein, 14 aa, synthetic construct
  SITE 1: note = Norleucine
  SITE 12: note = 5-fluorotryptophan
  SITE 14: note = Amidated residue
  SITE 3..11: note = Intrachain disulfide bridge
XYCVGKYDVYCXFT

SEQ ID NO: 60 - protein, 14 aa, synthetic construct
  SITE 1: note = Norleucine
  SITE 12: note = 7-aza-tryptophan
  SITE 14: note = Amidated residue
  SITE 3..11: note = Intrachain disulfide bridge
XYCVGKYDVYCXFT

SEQ ID NO: 61 - protein, 15 aa, synthetic construct
  SITE 1: note = Norleucine
  SITE 4: note = (S)-2-amino-3,3-dimethylbutanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCWFTK

SEQ ID NO: 62 - protein, 15 aa, synthetic construct
  SITE 1: note = Norleucine
  SITE 9: note = (S)-2-amino-3,3-dimethylbutanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCWFTK

SEQ ID NO: 63 - protein, 15 aa, synthetic construct
  SITE 1: note = Norleucine
  SITE 6: note = Ornithine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGXYDVYCWFTK

SEQ ID NO: 64 - protein, 15 aa, synthetic construct
  SITE 1: note = Norleucine
  SITE 3: note = Homocysteine
  SITE 11: note = Homocysteine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYXVGKYDVYXWFTK

SEQ ID NO: 65 - protein, 15 aa, synthetic construct
  SITE 1: note = Norleucine
  SITE 6: note = Acetylated Lysine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFTK

SEQ ID NO: 66 - protein, 15 aa, synthetic construct
  SITE 1: note = Norleucine
  SITE 6: note = Norleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGXYDVYCWFTK

SEQ ID NO: 67 - protein, 15 aa, synthetic construct
  SITE 1: note = Homoleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFTK

SEQ ID NO: 68 - protein, 15 aa, synthetic construct
  SITE 1: note = Norleucine
  SITE 13: note = Homophenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWXTK

SEQ ID NO: 69 - protein, 15 aa, synthetic construct
  SITE 1: note = Norleucine
  SITE 13: note = 4-cyano phenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWXTK

SEQ ID NO: 70 - protein, 15 aa, synthetic construct
  SITE 1: note = Norleucine
  SITE 13: note = 3-cyanophenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWXTK

SEQ ID NO: 71 - protein, 15 aa, synthetic construct
  SITE 1: note = Norleucine
  SITE 4: note = (S)-alpha-aminobutyric acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCWFTK

SEQ ID NO: 72 - protein, 15 aa, synthetic construct
  SITE 1: note = Homoleucine
  SITE 4: note = (S)-2-amino-3,3-dimethylbutanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCWFTK

SEQ ID NO: 73 - protein, 15 aa, synthetic construct
  SITE 1: note = Homoleucine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFTK

SEQ ID NO: 74 - protein, 15 aa, synthetic construct
  SITE 1: note = Homoleucine
  SITE 5: note = Sarcosine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVXKYDVYCWFTK

SEQ ID NO: 75 - protein, 15 aa, synthetic construct
  SITE 1: note = Homoleucine
  SITE 4: note = (S)-2-amino-3,3-dimethylbutanoic acid
  SITE 5: note = Sarcosine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXXKYDVYCWFTK

SEQ ID NO: 76 - protein, 15 aa, synthetic construct
  SITE 1: note = Homoleucine
  SITE 4: note = (S)-2-amino-3,3-dimethylbutanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCXFTK

SEQ ID NO: 77 - protein, 15 aa, synthetic construct
  SITE 1: note = Homoleucine
  SITE 5: note = Sarcosine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVXKYDVYCXFTK

SEQ ID NO: 78 - protein, 15 aa, synthetic construct
  SITE 1: note = Homoleucine
  SITE 4: note = (S)-2-amino-3,3-dimethylbutanoic acid
  SITE 5: note = Sarcosine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXXKYDVYCXFTK

SEQ ID NO: 79 - protein, 15 aa, synthetic construct
  SITE 1: note = Norleucine
  SITE 6: note = 2-amino-3-(pyridin-4-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGXYDVYCWFTK

SEQ ID NO: 80 - protein, 15 aa, synthetic construct
  SITE 1: note = Norleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYNVYCWFTK

SEQ ID NO: 81 - protein, 15 aa, synthetic construct
  SITE 1: note = Norleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYSVYCWFTK

SEQ ID NO: 82 - protein, 15 aa, synthetic construct
  SITE 1: note = Norleucine
  SITE 13: note = (S)-2-amino-3-cyclohexylpropanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWXTK

SEQ ID NO: 83 - protein, 15 aa, synthetic construct
  SITE 1: note = Norleucine
  SITE 13: note = 3-aminomethyl phenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWXTK

SEQ ID NO: 84 - protein, 15 aa, synthetic construct
  SITE 1: note = Norleucine
  SITE 13: note = 4-aminomethyl phenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWXTK

SEQ ID NO: 85 - protein, 15 aa, synthetic construct
  SITE 1: note = Norleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGRYDVYCWFTK

SEQ ID NO: 86 - protein, 15 aa, synthetic construct
  SITE 1: note = Norleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFKK

SEQ ID NO: 87 - protein, 15 aa, synthetic construct
  SITE 1: note = Norleucine
  SITE 14: note = Ornithine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFXK

SEQ ID NO: 88 - protein, 15 aa, synthetic construct
  SITE 1: note = Norleucine
  SITE 14: note = (S)-2,3-diaminopropanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFXK

SEQ ID NO: 89 - protein, 15 aa, synthetic construct
  SITE 1: note = Norleucine
  SITE 14: note = 3-(4-piperidinyl)-alanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFXK

SEQ ID NO: 90 - protein, 15 aa, synthetic construct
  SITE 1: note = Norleucine
  SITE 14: note = D-Lysine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFKK

SEQ ID NO: 91 - protein, 15 aa, synthetic construct
  SITE 1: note = Norleucine
  SITE 14: note = D-Glutamic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFEK

SEQ ID NO: 92 - protein, 15 aa, synthetic construct
  SITE 1: note = Norleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFSK

SEQ ID NO: 93 - protein, 15 aa, synthetic construct
  SITE 1: note = Norleucine
  SITE 14: note = Allothreonine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFXK

SEQ ID NO: 94 - protein, 14 aa, synthetic construct
  SITE 1: note = Norleucine
  SITE 14: note = Amidated residue
  SITE 3..11: note = Intrachain disulfide bridge
XYCVGKYDVYCWFT

SEQ ID NO: 95 - protein, 15 aa, synthetic construct
  SITE 1: note = Norleucine
  SITE 4: note = N-methyl valine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCWFTK

SEQ ID NO: 96 - protein, 15 aa, synthetic construct
  SITE 1: note = Norleucine
  SITE 9: note = (S)-alpha-aminobutyric acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCWFTK

SEQ ID NO: 97 - protein, 15 aa, synthetic construct
  SITE 1: note = Norleucine
  SITE 9: note = N-methyl valine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCWFTK

SEQ ID NO: 98 - protein, 14 aa, synthetic construct
  SITE 1: note = Homoleucine
  SITE 13: note = Residue modified with 1,4,7,10-tetra-azacylcododecane-N,N,N,N-tetraacetic acid via the side chain
  SITE 14: note = Amidated residue
  SITE 3..11: note = Intrachain disulfide bridge
XYCVGKYDVYCWKT

SEQ ID NO: 99 - protein, 14 aa, synthetic construct
  SITE 1: note = Homoleucine
  SITE 12: note = Residue modified with 1,4,7,10-tetra-azacylcododecane-N,N,N,N-tetraacetic acid via the side chain
  SITE 14: note = Amidated residue
  SITE 3..11: note = Intrachain disulfide bridge
XYCVGKYDVYCKFT

SEQ ID NO: 100 - protein, 14 aa, synthetic construct
  SITE 1: note = Homoleucine
  SITE 5: note = D-Lysine modified with 1,4,7,10-tetra-azacylcododecane-N,N,N,N-tetraacetic acid
  SITE 14: note = Amidated residue
  SITE 3..11: note = Intrachain disulfide bridge
XYCVKKYDVYCWFT

SEQ ID NO: 101 - protein, 15 aa, synthetic construct
  SITE 1: note = Norleucine
  SITE 10: note = Homoarginine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVXCWFTK

SEQ ID NO: 102 - protein, 15 aa, synthetic construct
  SITE 1: note = Norleucine
  SITE 8: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XYCVGKYXVYCWFTK

SEQ ID NO: 103 - protein, 15 aa, synthetic construct
  SITE 1: note = Norleucine
  SITE 6: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XYCVGXYDVYCWFTK

SEQ ID NO: 104 - protein, 15 aa, synthetic construct
  SITE 1: note = Norleucine
  SITE 5: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XYCVXKYDVYCWFTK

SEQ ID NO: 105 - protein, 15 aa, synthetic construct
  SITE 1: note = Norleucine
  SITE 4: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XYCXGKYDVYCWFTK

SEQ ID NO: 106 - protein, 15 aa, synthetic construct
  SITE 1: note = Norleucine
  SITE 2: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XXCVGKYDVYCWFTK

SEQ ID NO: 107 - protein, 15 aa, synthetic construct
  SITE 1: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XYCVGKYDVYCWFTK

SEQ ID NO: 108 - protein, 15 aa, synthetic construct
  SITE 1: note = Norleucine
  SITE 13: note = Homoarginine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWXTK

SEQ ID NO: 109 - protein, 15 aa, synthetic construct
  SITE 1: note = Norleucine
  SITE 12: note = Homoarginine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFTK

SEQ ID NO: 110 - protein, 15 aa, synthetic construct
  SITE 1: note = Norleucine
  SITE 2: note = Homoarginine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGKYDVYCWFTK

SEQ ID NO: 111 - protein, 15 aa, synthetic construct
  SITE 1: note = Homoarginine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFTK

SEQ ID NO: 112 - protein, 15 aa, synthetic construct
  SITE 1: note = Norleucine
  SITE 13: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XYCVGKYDVYCWXTK

SEQ ID NO: 113 - protein, 15 aa, synthetic construct
  SITE 1: note = Norleucine
  SITE 12: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XYCVGKYDVYCXFTK

SEQ ID NO: 114 - protein, 15 aa, synthetic construct
  SITE 1: note = Norleucine
  SITE 10: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XYCVGKYDVXCWFTK

SEQ ID NO: 115 - protein, 15 aa, synthetic construct
  SITE 1: note = Norleucine
  SITE 9: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XYCVGKYDXYCWFTK

SEQ ID NO: 116 - protein, 15 aa, synthetic construct
  SITE 1: note = Norleucine
  SITE 7: note = Homoglutamic acid
  SITE 15: note = D-Lysine modified with 1,4,7,10-tetra-azacylcododecane-N,N,N,N-tetraacetic acid
  SITE 3..11: note = Intrachain disulfide bridge
XYCVGKXDVYCWFTK

SEQ ID NO: 117 - protein, 15 aa, synthetic construct
  SITE 1: note = Homoleucine
  SITE 3: note = Penicillamine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYXVGKYDVYCWFTK

SEQ ID NO: 118 - protein, 15 aa, synthetic construct
  SITE 1: note = Homoleucine
  SITE 11: note = Penicillamine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYXWFTK

SEQ ID NO: 119 - protein, 15 aa, synthetic construct
  SITE 1: note = Homoleucine
  SITE 4: note = (S)-2-amino-2-cyclopropylacetic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCWFTK

SEQ ID NO: 120 - protein, 15 aa, synthetic construct
  SITE 1: note = Homoleucine
  SITE 4: note = Allothreonine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCWFTK

SEQ ID NO: 121 - protein, 15 aa, synthetic construct
  SITE 1: note = Homoleucine
  SITE 4: note = Alloisoleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCWFTK

SEQ ID NO: 122 - protein, 15 aa, synthetic construct
  SITE 1: note = Homoleucine
  SITE 4: note = 3-hydroxyvaline
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCWFTK

SEQ ID NO: 123 - protein, 15 aa, synthetic construct
  SITE 1: note = Homoleucine
  SITE 9: note = (S)-2-amino-2-cyclopropylacetic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCWFTK

SEQ ID NO: 124 - protein, 15 aa, synthetic construct
  SITE 1: note = Homoleucine
  SITE 9: note = 2-aminoisobutyric acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCWFTK

SEQ ID NO: 125 - protein, 15 aa, synthetic construct
  SITE 1: note = Homoleucine
  SITE 9: note = Allothreonine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCWFTK

SEQ ID NO: 126 - protein, 15 aa, synthetic construct
  SITE 1: note = Homoleucine
  SITE 9: note = Alloisoleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCWFTK

SEQ ID NO: 127 - protein, 15 aa, synthetic construct
  SITE 1: note = Homoleucine
  SITE 9: note = 3-hydroxyvaline
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCWFTK

SEQ ID NO: 128 - protein, 15 aa, synthetic construct
  SITE 1: note = Norleucine
  SITE 12: note = (S)-2-amino-3-(naphthalen-1-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFTK

SEQ ID NO: 129 - protein, 15 aa, synthetic construct
  SITE 1: note = Norleucine
  SITE 12: note = (S)-2-amino-3-(naphthalen-2-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFTK

SEQ ID NO: 130 - protein, 15 aa, synthetic construct
  SITE 1: note = Norleucine
  SITE 15: note = Amidated D-Lysine modified with 1,4,7,10-tetra-azacylcododecane-N,N,N,N-tetraacetic acid via the side chain
  SITE 3..11: note = Intrachain thioacetal bridge
XYCVGKYDVYCWFTK

SEQ ID NO: 131 - protein, 15 aa, synthetic construct
  SITE 1: note = Norleucine
  SITE 14: note = (S)-2,4-diaminobutanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFXK

SEQ ID NO: 132 - protein, 15 aa, synthetic construct
  SITE 1: note = Norleucine
  SITE 12: note = 1-methyl-tryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFTK

SEQ ID NO: 133 - protein, 15 aa, synthetic construct
  SITE 1: note = Norleucine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFTK

SEQ ID NO: 134 - protein, 15 aa, synthetic construct
  SITE 1: note = Norleucine
  SITE 12: note = 7-aza-tryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFTK

SEQ ID NO: 135 - protein, 15 aa, synthetic construct
  SITE 1: note = Homoleucine
  SITE 7: note = 3-(4-piperidinyl)-Ala
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCWFTK

SEQ ID NO: 136 - protein, 15 aa, synthetic construct
  SITE 1: note = Homoleucine
  SITE 7: note = (S)-2-amino-3-(4-(2-aminoethoxy)phenyl)propanoic acid modified with 1,4,7,10-tetra-azacylcododecane-N,N,N,N-tetraacetic acid
  SITE 15: note = Amidated D-Alanine
  SITE 3..11: note = Intrachain disulfide bridge
XYCVGKXDVYCWFTA

SEQ ID NO: 137 - protein, 15 aa, synthetic construct
  SITE 1: note = Homoleucine
  SITE 2: note = (S)-2-amino-3-(4-(2-aminoethoxy)phenyl)propanoic acid modified with 1,4,7,10-tetra-azacylcododecane-N,N,N,N-tetraacetic acid
  SITE 15: note = Amidated D-Alanine
  SITE 3..11: note = Intrachain disulfide bridge
XXCVGKYDVYCWFTA

SEQ ID NO: 138 - protein, 15 aa, synthetic construct
  SITE 1: note = Homoleucine
  SITE 4: note = (S)-2-amino-3,3-dimethylbutanoic acid
  SITE 6: note = Ornithine
  SITE 9: note = (S)-2-amino-2-cyclohexylacetic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGXYDXYCWFTK

SEQ ID NO: 139 - protein, 15 aa, synthetic construct
  SITE 1: note = Homoleucine
  SITE 4: note = (S)-2-amino-3,3-dimethylbutanoic acid
  SITE 7: note = 4-carboxyphenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVYCWFTK

SEQ ID NO: 140 - protein, 15 aa, synthetic construct
  SITE 1: note = Homoleucine
  SITE 4: note = (S)-2-amino-3,3-dimethylbutanoic acid
  SITE 5: note = Sarcosine
  SITE 7: note = 4-carboxyphenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXXKXDVYCWFTK

SEQ ID NO: 141 - protein, 15 aa, synthetic construct
  SITE 1: note = Homoleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFPK

SEQ ID NO: 142 - protein, 14 aa, synthetic construct
  SITE 1: note = Homoleucine
  SITE 10: note = Residue modified with 1,4,7,10-tetra-azacylcododecane-N,N,N,N-tetraacetic acid via the side chain
  SITE 14: note = Amidated residue
  SITE 3..11: note = Intrachain disulfide bridge
XYCVGKYDVKCWFT

SEQ ID NO: 143 - protein, 14 aa, synthetic construct
  SITE 1: note = Homoleucine
  SITE 9: note = Residue modified with 1,4,7,10-tetra-azacylcododecane-N,N,N,N-tetraacetic acid via the side chain
  SITE 14: note = Amidated residue
  SITE 3..11: note = Intrachain disulfide bridge
XYCVGKYDKYCWFT

SEQ ID NO: 144 - protein, 14 aa, synthetic construct
  SITE 1: note = Homoleucine
  SITE 7: note = Residue modified with 1,4,7,10-tetra-azacylcododecane-N,N,N,N-tetraacetic acid via the side chain
  SITE 14: note = Amidated residue
  SITE 3..11: note = Intrachain disulfide bridge
XYCVGKKDVYCWFT

SEQ ID NO: 145 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 14: note = Amidated residue
  SITE 3..11: note = Intrachain disulfide bridge
XYCVGKYDVYCWFT

SEQ ID NO: 146 - protein, 15 aa, synthetic construct
  SITE 1: note = Norleucine
  SITE 4: note = Alloisoleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCWFTK

SEQ ID NO: 147 - protein, 15 aa, synthetic construct
  SITE 1: note = Homoleucine
  SITE 12: note = 5-fluorotryptophan
  SITE 14: note = Alpha-methyl-serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFXK

SEQ ID NO: 148 - protein, 15 aa, synthetic construct
  SITE 1: note = Homoleucine
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFXK

SEQ ID NO: 149 - protein, 15 aa, synthetic construct
  SITE 1: note = Homoleucine
  SITE 14: note = 3-hydroxyvaline
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFXK

SEQ ID NO: 150 - protein, 15 aa, synthetic construct
  SITE 1: note = Homoleucine
  SITE 12: note = 1-methyl-tryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCWFTK

SEQ ID NO: 151 - protein, 15 aa, synthetic construct
  SITE 1: note = Homoleucine
  SITE 10: note = 3-fluorotyrosine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVXCWFTK

SEQ ID NO: 152 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 9: note = (S)-2-amino-2-cyclohexylacetic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDXYCWFTK

SEQ ID NO: 153 - protein, 15 aa, synthetic construct
  SITE 1: note = Homoleucine
  SITE 7: note = 2-amino-3-(pyridin-4-yl)propanoic acid
  SITE 9: note = (S)-2-amino-2-cyclohexylacetic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDXYCWFTK

SEQ ID NO: 154 - protein, 15 aa, synthetic construct
  SITE 1: note = Homoleucine
  SITE 6: note = 3-(4-piperidinyl)-Ala
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGXYDVYCWFTK

SEQ ID NO: 155 - protein, 15 aa, synthetic construct
  SITE 1: note = Homoleucine
  SITE 7: note = 2-amino-3-(pyridin-4-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCWFTK

SEQ ID NO: 156 - protein, 15 aa, synthetic construct
  SITE 1: note = Homoleucine
  SITE 12: note = 7-aza-tryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFTK

SEQ ID NO: 157 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVYCWFTK

SEQ ID NO: 158 - protein, 15 aa, synthetic construct
  SITE 1: note = Homoleucine
  SITE 12: note = (S)-2-amino-3-(naphthalen-1-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFTK

SEQ ID NO: 159 - protein, 15 aa, synthetic construct
  SITE 1: note = Homoleucine
  SITE 12: note = (S)-2-amino-3-(naphthalen-2-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFTK

SEQ ID NO: 160 - protein, 15 aa, synthetic construct
  SITE 1: note = Homoleucine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFPK

SEQ ID NO: 161 - protein, 15 aa, synthetic construct
  SITE 1: note = Homoleucine
  SITE 12: note = 5-fluorotryptophan
  SITE 14: note = 4-hydroxy-proline
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFXK

SEQ ID NO: 162 - protein, 15 aa, synthetic construct
  SITE 1: note = Homoleucine
  SITE 12: note = 5-fluorotryptophan
  SITE 14: note = Allothreonine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFXK

SEQ ID NO: 163 - protein, 15 aa, synthetic construct
  SITE 1: note = Homoleucine
  SITE 12: note = 5-fluorotryptophan
  SITE 14: note = D-Threonine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFTK

SEQ ID NO: 164 - protein, 15 aa, synthetic construct
  SITE 1: note = Homoleucine
  SITE 12: note = 5-fluorotryptophan
  SITE 13: note = 2-fluorophenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXXTK

SEQ ID NO: 165 - protein, 15 aa, synthetic construct
  SITE 1: note = Homoleucine
  SITE 12: note = 5-fluorotryptophan
  SITE 13: note = 2-methylphenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXXTK

SEQ ID NO: 166 - protein, 15 aa, synthetic construct
  SITE 1: note = Homoleucine
  SITE 12: note = 5-fluorotryptophan
  SITE 13: note = 4-carboxyphenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXXTK

SEQ ID NO: 167 - protein, 15 aa, synthetic construct
  SITE 1: note = Homoleucine
  SITE 7: note = Homotyrosine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCXFTK

SEQ ID NO: 168 - protein, 15 aa, synthetic construct
  SITE 1: note = Homoleucine
  SITE 12: note = 5-fluorotryptophan
  SITE 13: note = Alpha-methyl-phenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXXTK

SEQ ID NO: 169 - protein, 14 aa, synthetic construct
  SITE 1: note = Homoleucine
  SITE 4: note = 3-(4-piperidinyl)-Ala
  SITE 6: note = Residue modified with 1,4,7,10-tetra-azacylcododecane-N,N,N,N-tetraacetic acid via the side chain
  SITE 12: note = 5-fluorotryptophan
  SITE 14: note = Amidated residue
  SITE 3..11: note = Intrachain disulfide bridge
XYCXGKYDVYCXFT

SEQ ID NO: 170 - protein, 15 aa, synthetic construct
  SITE 1: note = Homoleucine
  SITE 10: note = 3-(3-pyridyl)-L-alanine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVXCXFTK

SEQ ID NO: 171 - protein, 15 aa, synthetic construct
  SITE 1: note = Homoleucine
  SITE 10: note = (S)-2-amino-3-(4-methoxyphenyl)propanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVXCXFTK

SEQ ID NO: 172 - protein, 15 aa, synthetic construct
  SITE 1: note = Homoleucine
  SITE 10: note = 4-fluorophenylalanine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVXCXFTK

SEQ ID NO: 173 - protein, 15 aa, synthetic construct
  SITE 1: note = Homoleucine
  SITE 12: note = 5-fluorotryptophan
  SITE 13: note = 3-fluorophenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXXTK

SEQ ID NO: 174 - protein, 15 aa, synthetic construct
  SITE 1: note = Homoleucine
  SITE 4: note = (S)-2-amino-4,4-dimethylpentanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCXFTK

SEQ ID NO: 175 - protein, 15 aa, synthetic construct
  SITE 1: note = Homoleucine
  SITE 12: note = 5-fluorotryptophan
  SITE 13: note = Alpha-methyl-norleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXXTK

SEQ ID NO: 176 - protein, 15 aa, synthetic construct
  SITE 1: note = Homoleucine
  SITE 12: note = 5-fluorotryptophan
  SITE 13: note = N-methyl isoleucine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXXTK

SEQ ID NO: 177 - protein, 15 aa, synthetic construct
  SITE 1: note = Homoleucine
  SITE 4: note = (S)-2-amino-3,3-dimethylbutanoic acid
  SITE 12: note = Alpha-methyl-tryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCXFTK

SEQ ID NO: 178 - protein, 15 aa, synthetic construct
  SITE 1: note = Homoleucine
  SITE 7: note = Allothreonine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCXFTK

SEQ ID NO: 179 - protein, 15 aa, synthetic construct
  SITE 1: note = Homoleucine
  SITE 4: note = (S)-2-amino-3,3-dimethylbutanoic acid
  SITE 12: note = (S)-2-amino-3-(naphthalen-1-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCXFTK

SEQ ID NO: 180 - protein, 15 aa, synthetic construct
  SITE 1: note = Homoleucine
  SITE 4: note = (S)-2-amino-3,3-dimethylbutanoic acid
  SITE 9: note = (S)-2-amino-2-cyclohexylacetic acid
  SITE 12: note = (S)-2-amino-3-(naphthalen-1-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDXYCXFTK

SEQ ID NO: 181 - protein, 15 aa, synthetic construct
  SITE 1: note = Homoleucine
  SITE 4: note = (S)-2-amino-3,3-dimethylbutanoic acid
  SITE 9: note = (S)-2-amino-2-cyclohexylacetic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDXYCXFTK

SEQ ID NO: 182 - protein, 14 aa, synthetic construct
  SITE 1: note = Homoleucine
  SITE 7: note = (S)-2-amino-3-(3-(aminomethyl)phenyl)propanoic acid modified with 1,4,7,10-tetra-azacylcododecane-N,N,N,N-tetraacetic acid
  SITE 12: note = 5-fluorotryptophan
  SITE 14: note = Amidated residue
  SITE 3..11: note = Intrachain disulfide bridge
XYCVGKXDVYCXFT

SEQ ID NO: 183 - protein, 14 aa, synthetic construct
  SITE 1: note = Homoleucine
  SITE 10: note = (S)-2-amino-3-(3-(aminomethyl)phenyl)propanoic acid modified with 1,4,7,10-tetra-azacylcododecane-N,N,N,N-tetraacetic acid
  SITE 12: note = 5-fluorotryptophan
  SITE 14: note = Amidated residue
  SITE 3..11: note = Intrachain disulfide bridge
XYCVGKYDVXCXFT

SEQ ID NO: 184 - protein, 15 aa, synthetic construct
  SITE 1: note = Homoleucine
  SITE 4: note = (S)-2-aminopentanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCXFTK

SEQ ID NO: 185 - protein, 15 aa, synthetic construct
  SITE 1: note = Homoleucine
  SITE 9: note = (S)-2-aminopentanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCXFTK

SEQ ID NO: 186 - protein, 15 aa, synthetic construct
  SITE 1: note = Homoleucine
  SITE 9: note = Norleucine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCXFTK

SEQ ID NO: 187 - protein, 15 aa, synthetic construct
  SITE 1: note = Homoleucine
  SITE 11: note = D-Cysteine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain thioacetal bridge
XYCVGKYDVYCXFTK

SEQ ID NO: 188 - protein, 15 aa, synthetic construct
  SITE 1: note = Homoleucine
  SITE 3: note = D-Cysteine
  SITE 11: note = D-Cysteine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain thioacetal bridge
XYCVGKYDVYCXFTK

SEQ ID NO: 189 - protein, 15 aa, synthetic construct
  SITE 1: note = Homoleucine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain thioacetal bridge
XYCVGKYDVYCXFTK

SEQ ID NO: 190 - protein, 15 aa, synthetic construct
  SITE 1: note = Homoleucine
  SITE 7: note = 2-amino-3-(pyridin-4-yl)propanoic acid
  SITE 9: note = (S)-2-amino-2-cyclohexylacetic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDXYCXFTK

SEQ ID NO: 191 - protein, 14 aa, synthetic construct
  SITE 1: note = Homoleucine
  SITE 10: note = (S)-2-amino-3-(4-(2-aminoethoxy)phenyl)propanoic acid modified with 1,4,7,10-tetra-azacylcododecane-N,N,N,N-tetraacetic acid
  SITE 12: note = 5-fluorotryptophan
  SITE 14: note = Amidated residue
  SITE 3..11: note = Intrachain disulfide bridge
XYCVGKYDVXCXFT

SEQ ID NO: 192 - protein, 15 aa, synthetic construct
  SITE 1: note = Homoleucine
  SITE 2: note = 4-fluorophenylalanine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGKYDVYCXFTK

SEQ ID NO: 193 - protein, 15 aa, synthetic construct
  SITE 1: note = Homoleucine
  SITE 2: note = (S)-2-amino-3-(4-methoxyphenyl)propanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGKYDVYCXFTK

SEQ ID NO: 194 - protein, 15 aa, synthetic construct
  SITE 1: note = Homoleucine
  SITE 2: note = Homotyrosine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGKYDVYCXFTK

SEQ ID NO: 195 - protein, 15 aa, synthetic construct
  SITE 1: note = Homoleucine
  SITE 2: note = Alpha-methyl-tyrosine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGKYDVYCXFTK

SEQ ID NO: 196 - protein, 15 aa, synthetic construct
  SITE 1: note = Homoleucine
  SITE 2: note = Alpha-methyl-norleucine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGKYDVYCXFTK

SEQ ID NO: 197 - protein, 15 aa, synthetic construct
  SITE 1: note = Homoleucine
  SITE 2: note = N-methyl isoleucine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGKYDVYCXFTK

SEQ ID NO: 198 - protein, 15 aa, synthetic construct
  SITE 1: note = Homoleucine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKTDVYCXFTK

SEQ ID NO: 199 - protein, 15 aa, synthetic construct
  SITE 1: note = Homoleucine
  SITE 7: note = Homoserine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCXFTK

SEQ ID NO: 200 - protein, 15 aa, synthetic construct
  SITE 1: note = Homoleucine
  SITE 7: note = (S)-2-amino-3-(tetrahydro-2H-pyran-4-yl)propanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCXFTK

SEQ ID NO: 201 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = (S)-2-amino-3-cyclohexylpropanoic acid
  SITE 12: note = 5-fluorotryptophan
  SITE 14: note = Amidated 3-hydroxyvaline
  SITE 3..11: note = Intrachain disulfide bridge
XYCVGKXDVYCXFX

SEQ ID NO: 202 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 3-(4-piperidinyl)-Ala
  SITE 12: note = 5-fluorotryptophan
  SITE 14: note = Amidated 3-hydroxyvaline
  SITE 3..11: note = Intrachain disulfide bridge
XYCVGKXDVYCXFX

SEQ ID NO: 203 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 3-(4-piperidinyl)-Ala
  SITE 12: note = 5-fluorotryptophan
  SITE 14: note = Amidated 3-hydroxyvaline
  SITE 3..11: note = Intrachain disulfide bridge
XYCIGKXDVYCXFX

SEQ ID NO: 204 - protein, 15 aa, synthetic construct
  SITE 1: note = Homoleucine
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XTCVGXYDVYCXFXK

SEQ ID NO: 205 - protein, 15 aa, synthetic construct
  SITE 1: note = Homoleucine
  SITE 12: note = 5-fluorotryptophan
  SITE 13: note = N-methyl phenylalanine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXXTK

SEQ ID NO: 206 - protein, 15 aa, synthetic construct
  SITE 1: note = Homoleucine
  SITE 7: note = (S)-2-amino-3-cyclohexylpropanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCXFTK

SEQ ID NO: 207 - protein, 15 aa, synthetic construct
  SITE 1: note = Homoleucine
  SITE 2: note = (S)-2-amino-3-(tetrahydro-2H-pyran-4-yl)propanoic acid
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGXYDVYCXFXK

SEQ ID NO: 208 - protein, 15 aa, synthetic construct
  SITE 1: note = Homoleucine
  SITE 2: note = (S)-2-amino-4,4-dimethylpentanoic acid
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGXYDVYCXFXK

SEQ ID NO: 209 - protein, 15 aa, synthetic construct
  SITE 1: note = Homoleucine
  SITE 2: note = Norleucine
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGXYDVYCXFXK

SEQ ID NO: 210 - protein, 15 aa, synthetic construct
  SITE 1: note = Homoleucine
  SITE 2: note = Allothreonine
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGXYDVYCXFXK

SEQ ID NO: 211 - protein, 15 aa, synthetic construct
  SITE 1: note = Homoleucine
  SITE 2: note = 3-hydroxyvaline
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGXYDVYCXFXK

SEQ ID NO: 212 - protein, 15 aa, synthetic construct
  SITE 1: note = Homoleucine
  SITE 2: note = (S)-2-amino-2-cyclohexylacetic acid
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGXYDVYCXFXK

SEQ ID NO: 213 - protein, 15 aa, synthetic construct
  SITE 1: note = Homoleucine
  SITE 2: note = (S)-2-amino-2-(tetrahydro-2H-pyran-4-yl)acetic acid
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGXYDVYCXFXK

SEQ ID NO: 214 - protein, 14 aa, synthetic construct
  SITE 1: note = Homoleucine
  SITE 2: note = (S)-2-amino-3-(4-(2-aminoethoxy)phenyl)propanoic acid modified with 1,4,7,10-tetra-azacylcododecane-N,N,N,N-tetraacetic acid
  SITE 12: note = 5-fluorotryptophan
  SITE 14: note = Amidated residue
  SITE 3..11: note = Intrachain disulfide bridge
XXCVGKYDVYCXFT

SEQ ID NO: 215 - protein, 15 aa, synthetic construct
  SITE 1: note = Homoleucine
  SITE 9: note = (S)-2-amino-2-cyclohexylacetic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDXYCXFTK

SEQ ID NO: 216 - protein, 15 aa, synthetic construct
  SITE 1: note = Homoleucine
  SITE 4: note = (S)-2-amino-3,3-dimethylbutanoic acid
  SITE 9: note = (S)-2-amino-2-cyclohexylacetic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDXYCWFTK

SEQ ID NO: 217 - protein, 15 aa, synthetic construct
  SITE 1: note = Homoleucine
  SITE 4: note = Norleucine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCXFTK

SEQ ID NO: 218 - protein, 14 aa, synthetic construct
  SITE 1: note = Homoleucine
  SITE 2: note = (S)-2-amino-3-(3-(aminomethyl)phenyl)propanoic acid modified with 1,4,7,10-tetra-azacylcododecane-N,N,N,N-tetraacetic acid
  SITE 12: note = 5-fluorotryptophan
  SITE 14: note = Amidated residue
  SITE 3..11: note = Intrachain disulfide bridge
XXCVGKYDVYCXFT

SEQ ID NO: 219 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVYCXFXK

SEQ ID NO: 220 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 10: note = (S)-2-amino-3-cyclohexylpropanoic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVXCXFXK

SEQ ID NO: 221 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 10: note = (S)-2-amino-4-cyanobutanoic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVXCXFXK

SEQ ID NO: 222 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVTCXFXK

SEQ ID NO: 223 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 10: note = (S)-2-amino-3-(tetrahydro-2H-pyran-4-yl)propanoic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVXCXFXK

SEQ ID NO: 224 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 10: note = (S)-2-amino-4,4-dimethylpentanoic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVXCXFXK

SEQ ID NO: 225 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 10: note = Norleucine
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVXCXFXK

SEQ ID NO: 226 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 10: note = Allothreonine
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVXCXFXK

SEQ ID NO: 227 - protein, 15 aa, synthetic construct
  SITE 1: note = Homoleucine
  SITE 6: note = 3-(4-piperidinyl)-Ala
  SITE 7: note = (S)-2-amino-3-cyclohexylpropanoic acid
  SITE 12: note = 5-fluorotryptophan
  SITE 14: note = 3-hydroxyvaline
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGXXDVYCXFXK

SEQ ID NO: 228 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = 5-fluorotryptophan
  SITE 14: note = Amidated N-methyl serine
  SITE 3..11: note = Intrachain disulfide bridge
XYCVGKXDVYCXFX

SEQ ID NO: 229 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = 5-fluorotryptophan
  SITE 14: note = Amidated 3-hydroxyvaline
  SITE 3..11: note = Intrachain disulfide bridge
XYCVGKXDVYCXFX

SEQ ID NO: 230 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = 5-fluorotryptophan
  SITE 14: note = Amidated 3-hydroxyvaline
  SITE 3..11: note = Intrachain disulfide bridge
XYCIGKXDVYCXFX

SEQ ID NO: 231 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 10: note = 3-hydroxyvaline
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVXCXFXK

SEQ ID NO: 232 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = (S)-2-amino-3-cyclohexylpropanoic acid
  SITE 12: note = 5-fluorotryptophan
  SITE 14: note = Amidated N-methyl serine
  SITE 3..11: note = Intrachain disulfide bridge
XYCIGKXDVYCXFX

SEQ ID NO: 233 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = (S)-2-amino-3-cyclohexylpropanoic acid
  SITE 12: note = 5-fluorotryptophan
  SITE 14: note = Amidated N-methyl serine
  SITE 3..11: note = Intrachain disulfide bridge
XYCVGKXDVYCXFX

SEQ ID NO: 234 - protein, 15 aa, synthetic construct
  SITE 1: note = Homoleucine
  SITE 2: note = (S)-2-amino-3-cyclobutylpropanoic acid
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XXCVGXYDVYCXFXK

SEQ ID NO: 235 - protein, 15 aa, synthetic construct
  SITE 1: note = Homoleucine
  SITE 4: note = (S)-2-amino-3,3-dimethylbutanoic acid
  SITE 12: note = 5-chlorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCXFTK

SEQ ID NO: 236 - protein, 15 aa, synthetic construct
  SITE 1: note = Homoleucine
  SITE 6: note = 3-(4-piperidinyl)-Ala
  SITE 7: note = (S)-2-amino-3-cyclohexylpropanoic acid
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGXXDVYCXFXK

SEQ ID NO: 237 - protein, 15 aa, synthetic construct
  SITE 1: note = Homoleucine
  SITE 3: note = N-methyl cysteine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYXVGKYDVYCXFTK

SEQ ID NO: 238 - protein, 15 aa, synthetic construct
  SITE 1: note = Homoleucine
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain thioacetal bridge
XACVGXYDVYCXFXK

SEQ ID NO: 239 - protein, 15 aa, synthetic construct
  SITE 1: note = Homoleucine
  SITE 6: note = 3-(4-piperidinyl)-Ala
  SITE 12: note = 5-fluorotryptophan
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain thioacetal bridge
XYCVGXYDVYCXFXK

SEQ ID NO: 240 - protein, 15 aa, synthetic construct
  SITE 1: note = Homoleucine
  SITE 7: note = 2-amino-3-(pyridin-4-yl)propanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCXFTK

SEQ ID NO: 241 - protein, 15 aa, synthetic construct
  SITE 1: note = Homoleucine
  SITE 3: note = Propargyl alanine
  SITE 11: note = (S)-2-amino-3-azidopropanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain triazole bridge
XYXVGKYDVYXXFTK

SEQ ID NO: 242 - protein, 15 aa, synthetic construct
  SITE 1: note = Homoleucine
  SITE 3: note = Propargyl alanine
  SITE 11: note = (S)-2-amino-3-azidopropanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain triazole bridge
XYXVGKYDVYXXFTK

SEQ ID NO: 243 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 10: note = (S)-2-amino-2-cyclohexylacetic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVXCXFXK

SEQ ID NO: 244 - protein, 15 aa, synthetic construct
  SITE 1: note = Homoleucine
  SITE 4: note = (S)-2-amino-3,3-dimethylbutanoic acid
  SITE 12: note = (S)-2-amino-3-(naphthalen-1-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCXFPK

SEQ ID NO: 245 - protein, 15 aa, synthetic construct
  SITE 1: note = Homoleucine
  SITE 12: note = (S)-2-amino-3-(naphthalen-1-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFPK

SEQ ID NO: 246 - protein, 15 aa, synthetic construct
  SITE 1: note = Homoleucine
  SITE 4: note = (S)-2-amino-3,3-dimethylbutanoic acid
  SITE 12: note = (S)-2-amino-3-(naphthalen-1-yl)propanoic acid
  SITE 14: note = Alpha-methyl-proline
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCXFXK

SEQ ID NO: 247 - protein, 15 aa, synthetic construct
  SITE 1: note = Homoleucine
  SITE 12: note = (S)-2-amino-3-(naphthalen-1-yl)propanoic acid
  SITE 14: note = Alpha-methyl-proline
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYDVYCXFXK

SEQ ID NO: 248 - protein, 15 aa, synthetic construct
  SITE 1: note = Homoleucine
  SITE 4: note = (S)-2-amino-3,3-dimethylbutanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKYDVYCXFPK

SEQ ID NO: 249 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 10: note = (S)-2-amino-3-(4-methoxyphenyl)propanoic acid
  SITE 12: note = 5-fluorotryptophan
  SITE 14: note = Amidated residue
  SITE 3..11: note = Intrachain disulfide bridge
XYCVGKXDVXCXFP

SEQ ID NO: 250 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = (S)-2-amino-3-(naphthalen-1-yl)propanoic acid
  SITE 14: note = Amidated cis-4-hydroxy-proline
  SITE 3..11: note = Intrachain disulfide bridge
XYCXGKXDVYCXFX

SEQ ID NO: 251 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = 5-fluorotryptophan
  SITE 14: note = Amidated cis-4-hydroxy-proline
  SITE 3..11: note = Intrachain disulfide bridge
XYCIGKXDVYCXFX

SEQ ID NO: 252 - protein, 14 aa, synthetic construct
  SITE 1: note = Homoleucine
  SITE 4: note = (S)-2-amino-3,3-dimethylbutanoic acid
  SITE 6: note = Residue modified with gamma-glutamic acid-1,4,7,10-tetra-azacylcododecane-N,N,N,N-tetraacetic acid via the side chain
  SITE 7: note = 2-amino-3-(pyridin-4-yl)propanoic acid
  SITE 12: note = (S)-2-amino-3-(naphthalen-1-yl)propanoic acid
  SITE 14: note = Amidated residue
  SITE 3..11: note = Intrachain disulfide bridge
XYCXGKXDVYCXFP

SEQ ID NO: 253 - protein, 14 aa, synthetic construct
  SITE 1: note = Homoleucine
  SITE 4: note = (S)-2-amino-3,3-dimethylbutanoic acid
  SITE 6: note = Residue modified with gamma-glutamic acid-gamma-glutamic acid-1,4,7,10-tetra-azacylcododecane-N,N,N,N-tetraacetic acid via the side chain
  SITE 7: note = 2-amino-3-(pyridin-4-yl)propanoic acid
  SITE 12: note = (S)-2-amino-3-(naphthalen-1-yl)propanoic acid
  SITE 14: note = Amidated residue
  SITE 3..11: note = Intrachain disulfide bridge
XYCXGKXDVYCXFP

SEQ ID NO: 254 - protein, 14 aa, synthetic construct
  SITE 1: note = Homoleucine
  SITE 4: note = (S)-2-amino-3,3-dimethylbutanoic acid
  SITE 7: note = (S)-2-amino-3-(3-(aminomethyl)phenyl)propanoic acid modified with gamma-glutamic acid-1,4,7,10-tetra-azacylcododecane-N,N,N,N-tetraacetic acid via the side chain
  SITE 12: note = (S)-2-amino-3-(naphthalen-1-yl)propanoic acid
  SITE 14: note = Amidated residue
  SITE 3..11: note = Intrachain disulfide bridge
XYCXGKXDVYCXFP

SEQ ID NO: 255 - protein, 14 aa, synthetic construct
  SITE 1: note = Homoleucine
  SITE 4: note = (S)-2-amino-3,3-dimethylbutanoic acid
  SITE 7: note = (S)-2-amino-3-(3-(aminomethyl)phenyl)propanoic acid modified with gamma-glutamic acid-gamma-glutamic acid-1,4,7,10-tetra-azacylcododecane-N,N,N,N-tetraacetic acid via the side chain
  SITE 12: note = (S)-2-amino-3-(naphthalen-1-yl)propanoic acid
  SITE 14: note = Amidated residue
  SITE 3..11: note = Intrachain disulfide bridge
XYCXGKXDVYCXFP

SEQ ID NO: 256 - protein, 14 aa, synthetic construct
  SITE 1: note = Homoleucine
  SITE 4: note = (S)-2-amino-3,3-dimethylbutanoic acid
  SITE 6: note = Residue modified with 4X 3-(2-aminoethoxy)propanoic acid-1,4,7,10-tetra-azacylcododecane-N,N,N,N-tetraacetic acid via the side chain
  SITE 7: note = 2-amino-3-(pyridin-4-yl)propanoic acid
  SITE 12: note = (S)-2-amino-3-(naphthalen-1-yl)propanoic acid
  SITE 14: note = Amidated residue
  SITE 3..11: note = Intrachain disulfide bridge
XYCXGKXDVYCXFP

SEQ ID NO: 257 - protein, 14 aa, synthetic construct
  SITE 1: note = Homoleucine
  SITE 4: note = (S)-2-amino-3,3-dimethylbutanoic acid
  SITE 7: note = (S)-2-amino-3-(3-(aminomethyl)phenyl)propanoic acid modified with 4x 3-(2-aminoethoxy)propanoic acid-1,4,7,10-tetra-azacylcododecane-N,N,N,N-tetraacetic acid via the side chain
  SITE 12: note = (S)-2-amino-3-(naphthalen-1-yl)propanoic acid
  SITE 14: note = Amidated residue
  SITE 3..11: note = Intrachain disulfide bridge
XYCXGKXDVYCXFP

SEQ ID NO: 258 - protein, 14 aa, synthetic construct
  SITE 1: note = Homoleucine
  SITE 4: note = (S)-2-amino-3,3-dimethylbutanoic acid
  SITE 7: note = (S)-2-amino-3-(3-(aminomethyl)phenyl)propanoic acid modified with gamma-glutamic acid-gamma-glutamic acid-gamma-glutamic acid-1,4,7,10-tetra-azacylcododecane-N,N,N,N-tetraacetic acid via the side chain
  SITE 12: note = (S)-2-amino-3-(naphthalen-1-yl)propanoic acid
  SITE 14: note = Amidated residue
  SITE 3..11: note = Intrachain disulfide bridge
XYCXGKXDVYCXFP

SEQ ID NO: 259 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = (S)-2-amino-3-(naphthalen-1-yl)propanoic acid
  SITE 15: note = Amidated D-Lysine modified with 4x 3-(2-aminoethoxy)propanoic acid-1,4,7,10-tetra-azacylcododecane-N,N,N,N-tetraacetic acid via the side chain
  SITE 3..11: note = Intrachain disulfide bridge
XYCXGKXDVYCXFPK

SEQ ID NO: 260 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = 5-fluorotryptophan
  SITE 14: note = Amidated residue
  SITE 3..11: note = Intrachain disulfide bridge
XYCIGKXDVYCXFP

SEQ ID NO: 261 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = Amidated residue
  SITE 3..11: note = Intrachain disulfide bridge
XYCIGKXDVYCXFP

SEQ ID NO: 262 - protein, 14 aa, synthetic construct
  SITE 1: note = Homoleucine
  SITE 7: note = (S)-2-amino-3-(3-(aminomethyl)phenyl)propanoic acid modified with 1,4,7,10-tetra-azacylcododecane-N,N,N,N-tetraacetic acid
  SITE 12: note = 5-fluorotryptophan
  SITE 3..11: note = Intrachain disulfide bridge
XYCVGKXDVYCXFP

SEQ ID NO: 263 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = (S)-2-amino-3-(naphthalen-1-yl)propanoic acid
  SITE 14: note = Amidated cis-4-fluoro-proline
  SITE 3..11: note = Intrachain disulfide bridge
XYCXGKXDVYCXFX

SEQ ID NO: 264 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = (S)-2-amino-3-(naphthalen-1-yl)propanoic acid
  SITE 14: note = Amidated trans-4-fluoro-proline
  SITE 3..11: note = Intrachain disulfide bridge
XYCXGKXDVYCXFX

SEQ ID NO: 265 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = (S)-2-amino-3-(naphthalen-1-yl)propanoic acid
  SITE 14: note = Amidated (S)-piperidine-2-carboxylic acid
  SITE 3..11: note = Intrachain disulfide bridge
XYCXGKXDVYCXFX

SEQ ID NO: 266 - protein, 14 aa, synthetic construct
  SITE 1: note = Homoleucine
  SITE 4: note = N-methyl isoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = 5-fluorotryptophan
  SITE 14: note = Amidated residue
  SITE 3..11: note = Intrachain disulfide bridge
XYCXGKXDVYCXFP

SEQ ID NO: 267 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = 5-fluorotryptophan
  SITE 14: note = Amidated trans-4-hydroxy-proline
  SITE 3..11: note = Intrachain disulfide bridge
XYCIGKXDVYCXFX

SEQ ID NO: 268 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = 5-fluorotryptophan
  SITE 14: note = Amidated cis-4-fluoro-proline
  SITE 3..11: note = Intrachain disulfide bridge
XYCIGKXDVYCXFX

SEQ ID NO: 269 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = 5-fluorotryptophan
  SITE 14: note = Amidated (S)-piperidine-2-carboxylic acid
  SITE 3..11: note = Intrachain disulfide bridge
XYCIGKXDVYCXFX

SEQ ID NO: 270 - protein, 15 aa, synthetic construct
  SITE 1: note = Homoleucine
  SITE 7: note = (S)-2-amino-3-(tetrahydro-2H-pyran-4-yl)propanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCXFPK

SEQ ID NO: 271 - protein, 17 aa, synthetic construct
  SITE 1: note = Gamma-glutamic acid modified with 1,4,7,10-tetra-azacylcododecane-N,N,N,N-tetraacetic acid
  SITE 2: note = Gamma-glutamic acid
  SITE 3: note = Gamma-glutamic acid
  SITE 4: note = Homoleucine
  SITE 5: note = 4-fluorophenylalanine
  SITE 7: note = (S)-2-amino-3,3-dimethylbutanoic acid
  SITE 9: note = 3-(4-piperidinyl)-Ala
  SITE 10: note = (S)-2-amino-3-(tetrahydro-2H-pyran-4-yl)propanoic acid
  SITE 15: note = (S)-2-amino-3-(naphthalen-1-yl)propanoic acid
  SITE 17: note = Amidated residue
  SITE 6..14: note = Intrachain disulfide bridge
EEEXXCXGXXDVYCXFP

SEQ ID NO: 272 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = Amidated residue
  SITE 3..11: note = Intrachain disulfide bridge
XYCXGKXDVYCXFP

SEQ ID NO: 273 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = (S)-2-amino-3-(naphthalen-1-yl)propanoic acid
  SITE 15: note = Amidated D-Lysine modified with gamma-glutamic acid-1,4,7,10-tetra-azacylcododecane-N,N,N,N-tetraacetic acid via the side chain
  SITE 3..11: note = Intrachain disulfide bridge
XYCXGKXDVYCXFPK

SEQ ID NO: 274 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = (S)-2-amino-3-(naphthalen-1-yl)propanoic acid
  SITE 15: note = Amidated D-Lysine modified with gamma-glutamic acid-gamma-glutamic acid-1,4,7,10-tetra-azacylcododecane-N,N,N,N-tetraacetic acid via the side chain
  SITE 3..11: note = Intrachain disulfide bridge
XYCXGKXDVYCXFPK

SEQ ID NO: 275 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = (S)-2-amino-3-(naphthalen-1-yl)propanoic acid
  SITE 15: note = Amidated D-Lysine modified with gamma-glutamic acid-gamma-glutamic acid-gamma-glutamic acid-1,4,7,10-tetra-azacylcododecane-N,N,N,N-tetraacetic acid via the side chain
  SITE 3..11: note = Intrachain disulfide bridge
XYCXGKXDVYCXFPK

SEQ ID NO: 276 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-cyclohexylpropanoic acid
  SITE 12: note = (S)-2-amino-3-(naphthalen-1-yl)propanoic acid
  SITE 15: note = Amidated D-Lysine modified with gamma-glutamic acid-1,4,7,10-tetra-azacylcododecane-N,N,N,N-tetraacetic acid via the side chain
  SITE 3..11: note = Intrachain disulfide bridge
XYCXGXXDVYCXFPK

SEQ ID NO: 277 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-cyclohexylpropanoic acid
  SITE 12: note = (S)-2-amino-3-(naphthalen-1-yl)propanoic acid
  SITE 15: note = Amidated D-Lysine modified with gamma-glutamic acid-gamma-glutamic acid-1,4,7,10-tetra-azacylcododecane-N,N,N,N-tetraacetic acid via the side chain
  SITE 3..11: note = Intrachain disulfide bridge
XYCXGXXDVYCXFPK

SEQ ID NO: 278 - protein, 14 aa, synthetic construct
  SITE 1: note = Homoleucine
  SITE 4: note = (S)-2-amino-3,3-dimethylbutanoic acid
  SITE 7: note = (S)-2-amino-3-(3-(aminomethyl)phenyl)propanoic acid modified with 8x 3-(2-aminoethoxy)propanoic acid-1,4,7,10-tetra-azacylcododecane-N,N,N,N-tetraacetic acid via the side chain
  SITE 12: note = (S)-2-amino-3-(naphthalen-1-yl)propanoic acid
  SITE 14: note = Amidated residue
  SITE 3..11: note = Intrachain disulfide bridge
XYCXGKXDVYCXFP

SEQ ID NO: 279 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = (S)-2-amino-3-(naphthalen-1-yl)propanoic acid
  SITE 15: note = Amidated D-Lysine modified with 8x 3-(2-aminoethoxy)propanoic acid-1,4,7,10-tetra-azacylcododecane-N,N,N,N-tetraacetic acid via the side chain
  SITE 3..11: note = Intrachain disulfide bridge
XYCXGKXDVYCXFPK

SEQ ID NO: 280 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-cyclohexylpropanoic acid
  SITE 12: note = (S)-2-amino-3-(naphthalen-1-yl)propanoic acid
  SITE 15: note = Amidated D-Lysine modified with 8x 3-(2-aminoethoxy)propanoic acid-1,4,7,10-tetra-azacylcododecane-N,N,N,N-tetraacetic acid via the side chain
  SITE 3..11: note = Intrachain disulfide bridge
XYCXGXXDVYCXFPK

SEQ ID NO: 281 - protein, 15 aa, synthetic construct
  SITE 1: note = Homoleucine
  SITE 7: note = (S)-2-amino-3-(tetrahydro-2H-pyran-4-yl)propanoic acid
  SITE 12: note = 5-fluorotryptophan
  SITE 15: note = Amidated D-Lysine modified with 4x 3-(2-aminoethoxy)propanoic acid-1,4,7,10-tetra-azacylcododecane-N,N,N,N-tetraacetic acid via the side chain
  SITE 3..11: note = Intrachain disulfide bridge
XYCVGKXDVYCXFPK

SEQ ID NO: 282 - protein, 14 aa, synthetic construct
  SITE 1: note = Homoleucine
  SITE 2: note = 4-fluorophenylalanine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = (S)-2-amino-3-(naphthalen-1-yl)propanoic acid
  SITE 14: note = Amidated residue
  SITE 3..11: note = Intrachain disulfide bridge
XXCXGXXDVYCXFP

SEQ ID NO: 283 - protein, 15 aa, synthetic construct
  SITE 1: note = Gamma-glutamic acid modified with 1,4,7,10-tetra-azacylcododecane-N,N,N,N-tetraacetic acid
  SITE 2: note = Homoleucine
  SITE 3: note = 7-aza-tryptophan
  SITE 5: note = (S)-2-amino-3,3-dimethylbutanoic acid
  SITE 7: note = 3-(4-piperidinyl)-Ala
  SITE 8: note = (S)-2-amino-3-(tetrahydro-2H-pyran-4-yl)propanoic acid
  SITE 13: note = (S)-2-amino-3-(naphthalen-1-yl)propanoic acid
  SITE 15: note = Amidated residue
  SITE 4..12: note = Intrachain disulfide bridge
EXXCXGXXDVYCXFP

SEQ ID NO: 284 - protein, 14 aa, synthetic construct
  SITE 1: note = Homoleucine
  SITE 6: note = 3-(4-piperidinyl)-Ala
  SITE 7: note = 4-carboxyphenylalanine
  SITE 12: note = 5-fluorotryptophan
  SITE 14: note = Amidated residue
  SITE 3..11: note = Intrachain disulfide bridge
XYCVGXXDVYCXFP

SEQ ID NO: 285 - protein, 15 aa, synthetic construct
  SITE 1: note = Homoleucine
  SITE 6: note = 3-(4-piperidinyl)-Ala
  SITE 7: note = 4-carboxyphenylalanine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGXXDVYCXFPK

SEQ ID NO: 286 - protein, 15 aa, synthetic construct
  SITE 1: note = Homoleucine
  SITE 7: note = 4-carboxyphenylalanine
  SITE 10: note = Norleucine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVXCXFPK

SEQ ID NO: 287 - protein, 15 aa, synthetic construct
  SITE 1: note = Homoleucine
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCVGKXDVYCXFPK

SEQ ID NO: 288 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 4-carboxyphenylalanine
  SITE 12: note = 5-fluorotryptophan
  SITE 14: note = Amidated residue
  SITE 3..11: note = Intrachain disulfide bridge
XYCVGKXDVYCXFP

SEQ ID NO: 289 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 4-carboxyphenylalanine
  SITE 12: note = 5-fluorotryptophan
  SITE 3..11: note = Intrachain disulfide bridge
XYCVGKXDVYCXFP

SEQ ID NO: 290 - protein, 15 aa, synthetic construct
  SITE 1: note = Homoleucine
  SITE 7: note = 4-carboxyphenylalanine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKXDVYCXFPK

SEQ ID NO: 291 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = Amidated residue
  SITE 3..11: note = Intrachain disulfide bridge
XYCVGKXDVYCXFP

SEQ ID NO: 292 - protein, 14 aa, synthetic construct
  SITE 1: note = Homoleucine
  SITE 7: note = 4-carboxyphenylalanine
  SITE 10: note = Norleucine
  SITE 12: note = 5-fluorotryptophan
  SITE 14: note = Amidated residue
  SITE 3..11: note = Intrachain disulfide bridge
XYCVGKXDVXCXFP

SEQ ID NO: 293 - protein, 14 aa, synthetic construct
  SITE 1: note = Homoleucine
  SITE 7: note = 4-carboxyphenylalanine
  SITE 12: note = 5-fluorotryptophan
  SITE 14: note = Amidated residue
  SITE 3..11: note = Intrachain disulfide bridge
XYCVGKXDVYCXFP

SEQ ID NO: 294 - protein, 14 aa, synthetic construct
  SITE 1: note = Homoleucine
  SITE 7: note = (S)-2-amino-3-(4-(2-aminoethoxy)phenyl)propanoic acid modified with 1,4,7,10-tetra-azacylcododecane-N,N,N,N-tetraacetic acid
  SITE 12: note = 5-fluorotryptophan
  SITE 14: note = Amidated residue
  SITE 3..11: note = Intrachain disulfide bridge
XYCVGKXDVYCXFT

SEQ ID NO: 295 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain thioacetal bridge
XYCXGKXDVYCXFXK

SEQ ID NO: 296 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain thioacetal bridge
XACXGKXDVYCXFXK

SEQ ID NO: 297 - protein, 15 aa, synthetic construct
  SITE 1: note = Homoleucine
  SITE 4: note = (S)-2-amino-3,3-dimethylbutanoic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain thioacetal bridge
XYCXGKADVYCXFXK

SEQ ID NO: 298 - protein, 15 aa, synthetic construct
  SITE 1: note = Homoleucine
  SITE 4: note = (S)-2-amino-3,3-dimethylbutanoic acid
  SITE 7: note = 2-amino-3-(pyridin-4-yl)propanoic acid
  SITE 12: note = (S)-2-amino-3-(naphthalen-1-yl)propanoic acid
  SITE 14: note = N-methyl serine
  SITE 15: note = Amidated D-Lysine modified with 1,4,7,10-tetra-azacylcododecane-N,N,N,N-tetraacetic acid via the side chain
  SITE 3..11: note = Intrachain thioacetal bridge
XYCXGKXAVYCXFXK

SEQ ID NO: 299 - protein, 14 aa, synthetic construct
  SITE 1: note = Homoleucine
  SITE 6: note = 3-(4-piperidinyl)-Ala
  SITE 7: note = (S)-2-amino-3-(4-(2-aminoethoxy)phenyl)propanoic acid modified with 1,4,7,10-tetra-azacylcododecane-N,N,N,N-tetraacetic acid
  SITE 12: note = 5-fluorotryptophan
  SITE 14: note = Amidated residue
  SITE 3..11: note = Intrachain disulfide bridge
XYCVGXXDVYCXFP

SEQ ID NO: 300 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 4-carboxyphenylalanine
  SITE 10: note = Norleucine
  SITE 12: note = 5-fluorotryptophan
  SITE 14: note = Amidated residue
  SITE 3..11: note = Intrachain disulfide bridge
XYCVGKXDVXCXFP

SEQ ID NO: 301 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFXK

SEQ ID NO: 302 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFPK

SEQ ID NO: 303 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 3..11: note = Intrachain disulfide bridge
XYCXGKXDVYCXFP

SEQ ID NO: 304 - protein, 14 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 3..11: note = Intrachain disulfide bridge
XYCXGKXDVYCXFP

SEQ ID NO: 305 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 15: note = D-Lysine modified with 4x 3-(2-aminoethoxy)propanoic acid-1,4,7,10-tetra-azacylcododecane-N,N,N,N-tetraacetic acid via the side chain
  SITE 3..11: note = Intrachain disulfide bridge
XYCXGKXDVYCXFPK

SEQ ID NO: 306 - protein, 15 aa, synthetic construct
  SITE 1: note = Homoleucine
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 4x 3-(2-aminoethoxy)propanoic acid-1,4,7,10-tetra-azacylcododecane-N,N,N,N-tetraacetic acid via the side chain
  SITE 3..11: note = Intrachain disulfide bridge
XYCIGKXDVYCXFXK

SEQ ID NO: 307 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 10: note = (S)-2-amino-3-(4-methoxyphenyl)propanoic acid
  SITE 12: note = (S)-2-amino-3-(naphthalen-1-yl)propanoic acid
  SITE 14: note = Amidated residue
  SITE 3..11: note = Intrachain disulfide bridge
XYCVGKXDVXCXFP

SEQ ID NO: 308 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 4x 3-(2-aminoethoxy)propanoic acid-1,4,7,10-tetra-azacylcododecane-N,N,N,N-tetraacetic acid via the side chain
  SITE 3..11: note = Intrachain disulfide bridge
XYCXGKXDVYCXFXK

SEQ ID NO: 309 - protein, 15 aa, synthetic construct
  SITE 1: note = Homoleucine
  SITE 4: note = (S)-2-amino-3,3-dimethylbutanoic acid
  SITE 6: note = Homoserine
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFPK

SEQ ID NO: 310 - protein, 15 aa, synthetic construct
  SITE 1: note = Homoleucine
  SITE 4: note = (S)-2-amino-3,3-dimethylbutanoic acid
  SITE 6: note = Ornithine
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFPK

SEQ ID NO: 311 - protein, 15 aa, synthetic construct
  SITE 1: note = Homoleucine
  SITE 6: note = 3-(4-piperidinyl)-Ala
  SITE 7: note = 4-carboxyphenylalanine
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCVGXXDVYCXFPK

SEQ ID NO: 312 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 4-carboxyphenylalanine
  SITE 12: note = (S)-2-amino-3-(naphthalen-1-yl)propanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGXXDVYCXFPK

SEQ ID NO: 313 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 4-carboxyphenylalanine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGXXDVYCXFPK

SEQ ID NO: 314 - protein, 15 aa, synthetic construct
  SITE 1: note = Homoleucine
  SITE 6: note = 3-(4-piperidinyl)-Ala
  SITE 7: note = 4-carboxyphenylalanine
  SITE 12: note = 5-fluorotryptophan
  SITE 15: note = Amidated D-Lysine modified with 4x 3-(2-aminoethoxy)propanoic acid-Lysine-1,4,7,10-tetra-azac ylcododecane-N,N,N,N-tetraacetic acid-gamma-glutamic acid-palmitoyl via the side chain
  SITE 3..11: note = Intrachain disulfide bridge
XYCVGXXDVYCXFPK

SEQ ID NO: 315 - protein, 15 aa, synthetic construct
  SITE 1: note = Homoleucine
  SITE 6: note = 3-(4-piperidinyl)-Ala
  SITE 7: note = 4-carboxyphenylalanine
  SITE 12: note = 5-fluorotryptophan
  SITE 15: note = Amidated D-Lysine modified with 24x 3-(2-aminoethoxy)propanoic acid-Lysine-1,4,7,10-tetra-azac ylcododecane-N,N,N,N-tetraacetic acid-gamma-glutamic acid-palmitoyl via the side chain
  SITE 3..11: note = Intrachain disulfide bridge
XYCVGXXDVYCXFPK

SEQ ID NO: 316 - protein, 15 aa, synthetic construct
  SITE 1: note = Homoleucine
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCIGXXDVYCXFXK

SEQ ID NO: 317 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFPK

SEQ ID NO: 318 - protein, 15 aa, synthetic construct
  SITE 1: note = Homoleucine
  SITE 6: note = (S)-2-amino-3-(1-(carboxymethyl)piperidin-4-yl)prop anoic acid
  SITE 7: note = 4-carboxyphenylalanine
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGXXDVYCXFPK

SEQ ID NO: 319 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 12: note = 5-fluorotryptophan
  SITE 13: note = Norleucine
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXPK

SEQ ID NO: 320 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 3..11: note = Intrachain disulfide bridge
XYCXGKXDVYCXFX

SEQ ID NO: 321 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 3-(4-piperidinyl)-Ala
  SITE 12: note = 5-fluorotryptophan
  SITE 14: note = 3-hydroxyvaline
  SITE 3..11: note = Intrachain disulfide bridge
XYCIGKXDVYCXFX

SEQ ID NO: 322 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = (S)-2-amino-3-(1-(carboxymethyl)piperidin-4-yl)prop anoic acid
  SITE 12: note = 5-fluorotryptophan
  SITE 14: note = 3-hydroxyvaline
  SITE 3..11: note = Intrachain disulfide bridge
XYCIGKXDVYCXFX

SEQ ID NO: 323 - protein, 14 aa, synthetic construct
  SITE 1: note = Homoleucine
  SITE 6: note = Residue modified with 1,4,7,10-tetra-azacylcododecane-N,N,N,N-tetraacetic acid via the side chain
  SITE 7: note = 4-carboxyphenylalanine
  SITE 12: note = 5-fluorotryptophan
  SITE 14: note = 3-hydroxyvaline
  SITE 3..11: note = Intrachain disulfide bridge
XYCVGKXDVYCXFX

SEQ ID NO: 324 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(1-(carboxymethyl)piperidin-4-yl)prop anoic acid
  SITE 12: note = 5-fluorotryptophan
  SITE 3..11: note = Intrachain disulfide bridge
XYCXGKXDVYCXFT

SEQ ID NO: 325 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 13: note = Norleucine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXXXK

SEQ ID NO: 326 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 3..11: note = Intrachain disulfide bridge
XYCXGKXDVYCXFT

SEQ ID NO: 327 - protein, 15 aa, synthetic construct
  SITE 1: note = Homoleucine
  SITE 3: note = (S)-2-aminohex-5-enoic acid
  SITE 11: note = (S)-2-aminohex-5-enoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain alkylene bridge
XYXVGKYDVYXXFTK

SEQ ID NO: 328 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = 2-amino-3-(pyridin-4-yl)propanoic acid
  SITE 12: note = (S)-2-amino-3-(naphthalen-1-yl)propanoic acid
  SITE 14: note = Amidated residue
  SITE 3..11: note = Intrachain thioacetal bridge
XYCXGKXDVYCXFP

SEQ ID NO: 329 - protein, 15 aa, synthetic construct
  SITE 1: note = Homoleucine
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCVGKXDVYCXFTK

SEQ ID NO: 330 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFTK

SEQ ID NO: 331 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 13: note = Norleucine
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXXTK

SEQ ID NO: 332 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXLTK

SEQ ID NO: 333 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 13: note = (S)-2-amino-3,3-dimethylbutanoic acid
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXXTK

SEQ ID NO: 334 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 13: note = Norleucine
  SITE 14: note = Amidated 3-hydroxyvaline
  SITE 3..11: note = Intrachain disulfide bridge
XYCXGKXDVYCXXX

SEQ ID NO: 335 - protein, 15 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Lysine
  SITE 3..11: note = Intrachain disulfide bridge
XYCXGKXDVYCXFXK

SEQ ID NO: 336 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = Amidated 3-hydroxyvaline
  SITE 3..11: note = Intrachain disulfide bridge
XYCXGKXDVYCXFX

SEQ ID NO: 337 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 338 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 339 - protein, 15 aa, synthetic construct
  SITE 1: note = Homoleucine
  SITE 4: note = (S)-2-amino-3,3-dimethylbutanoic acid
  SITE 6: note = Residue modified with carboxylated 21x 3-(2-aminoethoxy)propanoic acid
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFTK

SEQ ID NO: 340 - protein, 14 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 3..11: note = Intrachain disulfide bridge
XYCXGXXDVYCXFX

SEQ ID NO: 341 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(1-(carboxymethyl)piperidin-4-yl)prop anoic acid
  SITE 12: note = 5-fluorotryptophan
  SITE 14: note = 3-hydroxyvaline
  SITE 3..11: note = Intrachain disulfide bridge
XYCXGKXDVYCXFX

SEQ ID NO: 342 - protein, 14 aa, synthetic construct
  SITE 1: note = Homoleucine
  SITE 4: note = (S)-2-amino-3,3-dimethylbutanoic acid
  SITE 7: note = 3-(4-piperidinyl)-alanine modified with 1,4,7,10-tetra-azacylcododecane-N,N,N,N-tetraacetic acid via the side chain
  SITE 12: note = 5-fluorotryptophan
  SITE 14: note = 3-hydroxyvaline
  SITE 3..11: note = Intrachain disulfide bridge
XYCXGKXDVYCXFX

SEQ ID NO: 343 - protein, 15 aa, synthetic construct
  SITE 1: note = Homoleucine
  SITE 4: note = (S)-2-amino-3,3-dimethylbutanoic acid
  SITE 7: note = Alpha-methyl-leucine
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFTK

SEQ ID NO: 344 - protein, 15 aa, synthetic construct
  SITE 1: note = Homoleucine
  SITE 5: note = Sarcosine
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCVXKXDVYCXFXK

SEQ ID NO: 345 - protein, 15 aa, synthetic construct
  SITE 1: note = Homoleucine
  SITE 4: note = (S)-2-amino-3,3-dimethylbutanoic acid
  SITE 5: note = Sarcosine
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXXKXDVYCXFXK

SEQ ID NO: 346 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGPXDVYCXFXK

SEQ ID NO: 347 - protein, 15 aa, synthetic construct
  SITE 1: note = Homoleucine
  SITE 4: note = (S)-2-amino-3,3-dimethylbutanoic acid
  SITE 6: note = (S)-piperidine-2-carboxylic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 348 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 11: note = (2-mercaptoethyl)glycine
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYGXFTK

SEQ ID NO: 349 - protein, 15 aa, synthetic construct
  SITE 1: note = Homoleucine
  SITE 4: note = (S)-2-amino-3,3-dimethylbutanoic acid
  SITE 6: note = N-methyl lysine
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFTK

SEQ ID NO: 350 - protein, 15 aa, synthetic construct
  SITE 1: note = Homoleucine
  SITE 4: note = (S)-2-amino-3,3-dimethylbutanoic acid
  SITE 6: note = Alpha-methyl-lysine
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFTK

SEQ ID NO: 351 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 9: note = Alpha-methyl-valine
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDXYCXFTK

SEQ ID NO: 352 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 10: note = N-methyl tyrosine
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVXCXFTK

SEQ ID NO: 353 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 10: note = Alpha-methyl-tyrosine
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVXCXFTK

SEQ ID NO: 354 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 10: note = Alpha-methyl-leucine
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVXCXFTK

SEQ ID NO: 355 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 11: note = N-methyl cysteine
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYXXFTK

SEQ ID NO: 356 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = Homophenylalanine
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFTK

SEQ ID NO: 357 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 13: note = (S)-2-amino-4,4-dimethylpentanoic acid
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXXTK

SEQ ID NO: 358 - protein, 15 aa, synthetic construct
  SITE 1: note = Homoleucine
  SITE 3: note = N-methyl cysteine
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYXVGKXDVYCXFTK

SEQ ID NO: 359 - protein, 15 aa, synthetic construct
  SITE 1: note = Homoleucine
  SITE 5: note = Sarcosine
  SITE 7: note = (S)-2-amino-3-(tetrahydro-2H-pyran-4-yl)propanoic acid
  SITE 12: note = 5-fluorotryptophan
  SITE 15: note = D-Lysine modified with 1,4,7,10-tetra-azacylcododecane-N,N,N,N-tetraacetic acid
  SITE 3..11: note = Intrachain disulfide bridge
XYCVXKXDVYCXFTK

SEQ ID NO: 360 - protein, 15 aa, synthetic construct
  SITE 1: note = Homoleucine
  SITE 4: note = (S)-2-amino-3,3-dimethylbutanoic acid
  SITE 6: note = Residue modified with gamma-glutamic acid-gamma-glutamic acid-gamma-glutamic acid-octadecanedioic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFXK

SEQ ID NO: 361 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-4-(4-iodophenyl)butanoic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 362 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFXK

SEQ ID NO: 363 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGKXDVYCXFXK

SEQ ID NO: 364 - protein, 15 aa, synthetic construct
  SITE 1: note = (S)-2-amino-3-cyclohexylpropanoic acid
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFXK

SEQ ID NO: 365 - protein, 15 aa, synthetic construct
  SITE 1: note = (S)-2-amino-4-cyclohexylbutanoic acid
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFXK

SEQ ID NO: 366 - protein, 15 aa, synthetic construct
  SITE 1: note = Homoleucine
  SITE 4: note = Alpha-methyl-valine
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFXK

SEQ ID NO: 367 - protein, 15 aa, synthetic construct
  SITE 1: note = Homoleucine
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCTGKXDVYCXFXK

SEQ ID NO: 368 - protein, 15 aa, synthetic construct
  SITE 1: note = Homoleucine
  SITE 4: note = (S)-2-amino-3,3-dimethylbutanoic acid
  SITE 6: note = (S)-2,4-diaminobutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 369 - protein, 15 aa, synthetic construct
  SITE 1: note = Homoleucine
  SITE 4: note = (S)-2-amino-3,3-dimethylbutanoic acid
  SITE 6: note = (S)-2,3-diaminopropanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 370 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 9: note = Homoserine
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDXYCXFXK

SEQ ID NO: 371 - protein, 15 aa, synthetic construct
  SITE 1: note = Homoleucine
  SITE 4: note = (S)-2-amino-3,3-dimethylbutanoic acid
  SITE 6: note = D-Proline
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGPPDVYCXFXK

SEQ ID NO: 372 - protein, 15 aa, synthetic construct
  SITE 1: note = Homoleucine
  SITE 4: note = (S)-2-amino-3,3-dimethylbutanoic acid
  SITE 6: note = (S)-azetidine-2-carboxylic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 373 - protein, 15 aa, synthetic construct
  SITE 1: note = Homoleucine
  SITE 4: note = (S)-2-amino-3,3-dimethylbutanoic acid
  SITE 6: note = D-Proline
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGPXDVYCXFXK

SEQ ID NO: 374 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDTYCXFXK

SEQ ID NO: 375 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDSYCXFXK

SEQ ID NO: 376 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 9: note = Alpha-methyl-serine
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDXYCXFXK

SEQ ID NO: 377 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 13: note = 3-fluorophenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXXXK

SEQ ID NO: 378 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 13: note = 4-fluorophenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXXXK

SEQ ID NO: 379 - protein, 14 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = Amidated 3-hydroxyvaline
  SITE 3..11: note = Intrachain disulfide bridge
XYCXGXXDVYCXFX

SEQ ID NO: 380 - protein, 14 aa, synthetic construct
  SITE 1: note = Homoleucine
  SITE 4: note = (S)-2-amino-3,3-dimethylbutanoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = Amidated 3-hydroxyvaline
  SITE 3..11: note = Intrachain disulfide bridge
XYCXGKXDVYCXFX

SEQ ID NO: 381 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Alanine
  SITE 3..11: note = Intrachain disulfide bridge
XYCXGXXDVYCXFXA

SEQ ID NO: 382 - protein, 15 aa, synthetic construct
  SITE 1: note = Homoleucine
  SITE 4: note = (S)-2-amino-3,3-dimethylbutanoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = Amidated D-Alanine
  SITE 3..11: note = Intrachain disulfide bridge
XYCXGKXDVYCXFXA

SEQ ID NO: 383 - protein, 15 aa, synthetic construct
  SITE 1: note = Homoleucine
  SITE 5: note = Sarcosine
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCTXKXDVYCXFXK

SEQ ID NO: 384 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 10: note = (S)-2-amino-3-(4-((fluorosulfonyl)oxy)phenyl)propan oic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVXCXFXK

SEQ ID NO: 385 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 8: note = (S)-2-amino-3-(4-((fluorosulfonyl)oxy)phenyl)propan oic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXXVYCXFXK

SEQ ID NO: 386 - protein, 14 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = Amidated 3-hydroxyvaline
  SITE 3..11: note = Intrachain disulfide bridge
XYCXGKXDVYCXFX

SEQ ID NO: 387 - protein, 16 aa, synthetic construct
  SITE 1: note = Residue modified with 1,4,7,10-tetra-azacylcododecane-N,N,N,N-tetraacetic acid
  SITE 2: note = 2-(2-aminoethoxy)acetic acid
  SITE 3: note = Homoleucine
  SITE 6: note = (S)-2-amino-3,3-dimethylbutanoic acid
  SITE 9: note = (S)-2-amino-3-(tetrahydro-2H-pyran-4-yl)propanoic acid
  SITE 14: note = 5-fluorotryptophan
  SITE 16: note = Amidated 3-hydroxyvaline
  SITE 5..13: note = Intrachain disulfide bridge
EXXYCXGKXDVYCXFX

SEQ ID NO: 388 - protein, 16 aa, synthetic construct
  SITE 1: note = Gamma-glutamic acid modified with 1,4,7,10-tetra-azacylcododecane-N,N,N,N-tetraacetic acid
  SITE 2: note = 2-(2-aminoethoxy)acetic acid
  SITE 3: note = Homoleucine
  SITE 6: note = (S)-2-amino-3,3-dimethylbutanoic acid
  SITE 9: note = (S)-2-amino-3-(tetrahydro-2H-pyran-4-yl)propanoic acid
  SITE 14: note = 5-fluorotryptophan
  SITE 16: note = Amidated 3-hydroxyvaline
  SITE 5..13: note = Intrachain disulfide bridge
EXXYCXGKXDVYCXFX

SEQ ID NO: 389 - protein, 14 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = Amidated 3-hydroxyvaline
  SITE 3..11: note = Intrachain disulfide bridge
XYCXGKXDVYCXFX

SEQ ID NO: 390 - protein, 14 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = Amidated 3-hydroxyvaline
  SITE 3..11: note = Intrachain disulfide bridge
XYCXGKXDVYCXFX

SEQ ID NO: 391 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 1-(carboxymethyl)-tryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCWFXK

SEQ ID NO: 392 - protein, 15 aa, synthetic construct
  SITE 1: note = (S)-2-amino-5,5-dimethylhexanoic acid
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFXK

SEQ ID NO: 393 - protein, 15 aa, synthetic construct
  SITE 1: note = (S)-2-amino-4-cyclobutylbutanoic acid
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFXK

SEQ ID NO: 394 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 10: note = 3-hydroxyphenylalanine
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVXCXFXK

SEQ ID NO: 395 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 10: note = 4-carbamoyl-phenylalanine
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVXCXFXK

SEQ ID NO: 396 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXHXK

SEQ ID NO: 397 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 13: note = 2-amino-3-(pyridin-4-yl)propanoic acid
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXXXK

SEQ ID NO: 398 - protein, 15 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 13: note = 3-(3-pyridyl)-L-alanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXXXK

SEQ ID NO: 399 - protein, 14 aa, synthetic construct
  SITE 1: note = Homoleucine
  SITE 4: note = (S)-2-amino-3,3-dimethylbutanoic acid
  SITE 7: note = (S)-2-amino-3-(tetrahydro-2H-pyran-4-yl)propanoic acid
  SITE 12: note = 5-fluorotryptophan
  SITE 14: note = Amidated 3-hydroxyvaline
  SITE 3..11: note = Intrachain disulfide bridge
XYCXGKXDVYCXFX

SEQ ID NO: 400 - protein, 15 aa, synthetic construct
  SITE 1: note = Homoleucine
  SITE 4: note = (S)-2-amino-3,3-dimethylbutanoic acid
  SITE 6: note = Residue modified with gamma-glutamic acid-hexadecanedioic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGKXDVYCXFXK

SEQ ID NO: 401 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Serine
  SITE 3..11: note = Intrachain disulfide bridge
XYCXGXXDVYCXFXS

SEQ ID NO: 402 - protein, 15 aa, synthetic construct
  SITE 1: note = Homoleucine
  SITE 4: note = (S)-2-amino-3,3-dimethylbutanoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = Amidated D-Serine
  SITE 3..11: note = Intrachain disulfide bridge
XYCXGKXDVYCXFXS

SEQ ID NO: 403 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGXXDVYCXFXE

SEQ ID NO: 404 - protein, 15 aa, synthetic construct
  SITE 1: note = Homoleucine
  SITE 4: note = (S)-2-amino-3,3-dimethylbutanoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGKXDVYCXFXE

SEQ ID NO: 405 - protein, 14 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(1-(carboxymethyl)piperidin-4-yl)prop anoic acid
  SITE 12: note = 5-fluorotryptophan
  SITE 14: note = Amidated 3-hydroxyvaline
  SITE 3..11: note = Intrachain disulfide bridge
XYCXGKXDVYCXFX

SEQ ID NO: 406 - protein, 15 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(1-(carboxymethyl)piperidin-4-yl)prop anoic acid
  SITE 12: note = 5-fluorotryptophan
  SITE 14: note = 3-hydroxyvaline
  SITE 15: note = Amidated D-Serine
  SITE 3..11: note = Intrachain disulfide bridge
XYCXGKXDVYCXFXS

SEQ ID NO: 407 - protein, 15 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(1-(carboxymethyl)piperidin-4-yl)prop anoic acid
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGKXDVYCXFXE

SEQ ID NO: 408 - protein, 15 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Serine
  SITE 3..11: note = Intrachain disulfide bridge
XYCXGKXDVYCXFXS

SEQ ID NO: 409 - protein, 15 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGKXDVYCXFXE

SEQ ID NO: 410 - protein, 15 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-((1s,4R)-4-hydroxycyclohexyl)propanoi c acid
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGKXDVYCXFXE

SEQ ID NO: 411 - protein, 15 aa, synthetic construct
  SITE 1: note = Homoleucine
  SITE 4: note = (S)-2-amino-3,3-dimethylbutanoic acid
  SITE 6: note = Residue modified with 1,4,7,10-tetra-azacylcododecane-N,N,N,N-tetraacetic acid via the side chain
  SITE 7: note = (S)-2-amino-3-((1r,4S)-4-hydroxycyclohexyl)propanoi c acid
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGKXDVYCXFXE

SEQ ID NO: 412 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 10: note = (S)-2-amino-3-(4-ureidophenyl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVXCXFXK

SEQ ID NO: 413 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with glutamic acid-1,4,7,10-tetra-azacylcododecane-N,N,N,N-tetraacetic acid via the side chain
  SITE 3..11: note = Intrachain disulfide bridge
XYCXGXXDVYCXFXK

SEQ ID NO: 414 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with D-Glutamic acid-1,4,7,10-tetra-azacylcododecane-N,N,N,N-tetraacetic acid via the side chain
  SITE 3..11: note = Intrachain disulfide bridge
XYCXGXXDVYCXFXK

SEQ ID NO: 415 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = 3-chlorophenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 416 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = 3-hydroxyphenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 417 - protein, 15 aa, synthetic construct
  SITE 1: note = Homoleucine
  SITE 4: note = Beta-methyl isoleucine
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 418 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 419 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3R)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 420 - protein, 15 aa, synthetic construct
  SITE 1: note = Homoleucine
  SITE 4: note = (S)-2-amino-3,3-dimethylbutanoic acid
  SITE 6: note = (S)-2,4-diaminobutanoic acid
  SITE 7: note = (S)-2-amino-3-(tetrahydro-2H-pyran-4-yl)propanoic acid
  SITE 12: note = (S)-2-amino-3-(naphthalen-1-yl)propanoic acid
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 421 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = (S)-2-amino-3-(naphthalen-1-yl)propanoic acid
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 422 - protein, 15 aa, synthetic construct
  SITE 1: note = Methylsulfonyl modified homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 423 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with R-2-(4,7,10-tris(carboxymeth yl)-1,4,7,10-tetraazacyclododecan-1-yl)pentanedioic acid via the side chain
  SITE 3..11: note = Intrachain disulfide bridge
XYCXGXXDVYCXFXK

SEQ ID NO: 424 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with S-2-(4,7,10-tris(carboxymeth yl)-1,4,7,10-tetraazacyclododecan-1-yl)pentanedioic acid via the side chain
  SITE 3..11: note = Intrachain disulfide bridge
XYCXGXXDVYCXFXK

SEQ ID NO: 425 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with gamma-glutamic acid-1,4,7,10-tetra-azacylcododecane-N,N,N,N-tetraacetic acid via the side chain
  SITE 3..11: note = Intrachain disulfide bridge
XYCXGXXDVYCXFXK

SEQ ID NO: 426 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = (S)-2-amino-3-(naphthalen-1-yl)propanoic acid
  SITE 14: note = 3-hydroxyvaline
  SITE 15: note = D-Lysine modified with gamma-glutamic acid-1,4,7,10-tetra-azacylcododecane-N,N,N,N-tetraacetic acid via the side chain
  SITE 3..11: note = Intrachain disulfide bridge
XYCXGXXDVYCXFXK

SEQ ID NO: 427 - protein, 15 aa, synthetic construct
  SITE 1: note = (S)-2-amino-4-cyclohexylbutanoic acid
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 428 - protein, 15 aa, synthetic construct
  SITE 1: note = (S)-2-amino-4-cyclobutylbutanoic acid
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 429 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGXXDVYCXFXE

SEQ ID NO: 430 - protein, 14 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = Amidated 3-hydroxyvaline
  SITE 3..11: note = Intrachain disulfide bridge
XYCXGXXDVYCXFX

SEQ ID NO: 431 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = Amidated D-Serine
  SITE 3..11: note = Intrachain disulfide bridge
XYCXGXXDVYCXFXS

SEQ ID NO: 432 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGXXDVYCXFXE

SEQ ID NO: 433 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = D-Glutamic acid
  SITE 3..11: note = Intrachain disulfide bridge
XYCXGXXDVYCXFXE

SEQ ID NO: 434 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = D-Serine
  SITE 3..11: note = Intrachain disulfide bridge
XYCXGXXDVYCXFXS

SEQ ID NO: 435 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 2S,3S-beta-methyl-5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 436 - protein, 16 aa, synthetic construct
  SITE 1: note = Homoleucine modified with succinic acid
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 4..12: note = Intrachain disulfide bridge
XXYCXGXXDVYCXFXK

SEQ ID NO: 437 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Lysine modified with gamma-glutamic acid-1,4,7,10-tetra-azacylcododecane-N,N,N,N-tetraacetic acid via the side chain
  SITE 3..11: note = Intrachain disulfide bridge
XYCXGXXDVYCXFXK

SEQ ID NO: 438 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = (S)-2-amino-3-(naphthalen-1-yl)propanoic acid
  SITE 14: note = 3-hydroxyvaline
  SITE 15: note = D-Alanine
  SITE 3..11: note = Intrachain disulfide bridge
XYCXGXXDVYCXFXA

SEQ ID NO: 439 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = (S)-2-amino-3-(naphthalen-1-yl)propanoic acid
  SITE 14: note = 3-hydroxyvaline
  SITE 15: note = Amidated D-Glutamic acid
  SITE 3..11: note = Intrachain disulfide bridge
XYCXGXXDVYCXFXE

SEQ ID NO: 440 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = D-Alanine
  SITE 3..11: note = Intrachain disulfide bridge
XYCXGXXDVYCXFXA

SEQ ID NO: 441 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = 2-methylphenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 442 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-chlorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 443 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 3-(2-(2-aminoethoxy)ethoxy)propanoic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGXXDVYCXFXE

SEQ ID NO: 444 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with gamma-glutamic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGXXDVYCXFXE

SEQ ID NO: 445 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with R-1,4,7,10-Tetraazacyclododecane-1,4,7,10-tetraacetic acid, alpha-(2-carboxyethyl) via the side chain
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGXXDVYCXFXE

SEQ ID NO: 446 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 4-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 447 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 1-methyl-tryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCWFXK

SEQ ID NO: 448 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-methoxytryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 449 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with homoglutamic acid-1,4,7,10-tetra-azacylcododecane-N,N,N,N-tetraacetic acid via the side chain
  SITE 3..11: note = Intrachain disulfide bridge
XYCXGXXDVYCXFXK

SEQ ID NO: 450 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 4x 3-(2-aminoethoxy)propanoic acid-1,4,7,10-tetra-azacylcododecane-N,N,N,N-tetraacetic acid via the side chain
  SITE 3..11: note = Intrachain disulfide bridge
XYCXGXXDVYCXFXK

SEQ ID NO: 451 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 10: note = 2-hydroxyphenylalanine
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVXCXFXK

SEQ ID NO: 452 - protein, 15 aa, synthetic construct
  SITE 1: note = Urea modified homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 453 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3R)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 454 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = 2-fluorophenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 455 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-methyltryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 456 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 4-chlorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 457 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 4-methyltryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 458 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 3-(2-(2-aminoethoxy)ethoxy)propanoic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGXXDVYCXFXE

SEQ ID NO: 459 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with gamma-glutamic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGXXDVYCXFXE

SEQ ID NO: 460 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with R-1,4,7,10-Tetraazacyclododecane-1,4,7,10-tetraacetic acid, alpha-(2-carboxyethyl) via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Amidated D-Glutamic acid
  SITE 3..11: note = Intrachain disulfide bridge
XYCXGXXDVYCXFXE

SEQ ID NO: 461 - protein, 16 aa, synthetic construct
  SITE 1: note = 5-oxoproline
  SITE 2: note = Homoleucine
  SITE 5: note = (S)-2-amino-3,3-dimethylbutanoic acid
  SITE 7: note = 3-(4-piperidinyl)-Ala
  SITE 8: note = (S)-2-amino-3-(tetrahydro-2H-pyran-4-yl)propanoic acid
  SITE 13: note = 5-fluorotryptophan
  SITE 15: note = 3-hydroxyvaline
  SITE 16: note = D-Lysine modified with 1,4,7,10-tetra-azacylcododecane-N,N,N,N-tetraacetic acid
  SITE 4..12: note = Intrachain disulfide bridge
XXYCXGXXDVYCXFXK

SEQ ID NO: 462 - protein, 15 aa, synthetic construct
  SITE 1: note = Homoleucine
  SITE 2: note = (S)-2-amino-3-(4-((fluorosulfonyl)oxy)phenyl)propan oic acid
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XXCXGXXDVYCXFXK

SEQ ID NO: 463 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-((1s,4R)-4-hydroxycyclohexyl)propanoi c acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 464 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-((1r,4S)-4-hydroxycyclohexyl)propanoi c acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 465 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Lysine modified with gamma-glutamic acid-tranexamic acid-1,4,7,10-tetra-azacylcododecane-N,N,N,N-tetraacetic acid via the side chain
  SITE 3..11: note = Intrachain disulfide bridge
XYCXGXXDVYCXFXK

SEQ ID NO: 466 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = Epsilon-Lysine modified with Val-Met-4-(aminomethyl)benzoic acid-1,4,7,10-tetra-azacylcododecane-N,N,N,N-tetraacetic acid via the side chain
  SITE 3..11: note = Intrachain disulfide bridge
XYCXGXXDVYCXFXK

SEQ ID NO: 467 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = D-Serine
  SITE 3..11: note = Intrachain disulfide bridge
XYCXGXXDVYCXFXS

SEQ ID NO: 468 - protein, 15 aa, synthetic construct
  SITE 1: note = Homoleucine
  SITE 4: note = (S)-2-amino-3,3-dimethylbutanoic acid
  SITE 6: note = (S)-2-amino-3-(tetrahydro-2H-pyran-4-yl)propanoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = D-Serine
  SITE 3..11: note = Intrachain disulfide bridge
XYCXGXXDVYCXFXS

SEQ ID NO: 469 - protein, 15 aa, synthetic construct
  SITE 1: note = (S)-2-amino-4-cyclohexylbutanoic acid
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Alanine
  SITE 3..11: note = Intrachain disulfide bridge
XYCXGXXDVYCXFXA

SEQ ID NO: 470 - protein, 15 aa, synthetic construct
  SITE 1: note = (S)-2-amino-4-cyclohexylbutanoic acid
  SITE 4: note = (S)-2-amino-3,3-dimethylbutanoic acid
  SITE 6: note = 3-(4-piperidinyl)-alanine modified with gamma-glutamic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Alanine
  SITE 3..11: note = Intrachain disulfide bridge
XYCXGXXDVYCXFXA

SEQ ID NO: 471 - protein, 15 aa, synthetic construct
  SITE 1: note = (S)-2-amino-4-cyclohexylbutanoic acid
  SITE 4: note = (S)-2-amino-3,3-dimethylbutanoic acid
  SITE 6: note = 3-(4-piperidinyl)-alanine modified with gamma-glutamic acid-gamma-glutamic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Alanine
  SITE 3..11: note = Intrachain disulfide bridge
XYCXGXXDVYCXFXA

SEQ ID NO: 472 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = D-Alanine
  SITE 3..11: note = Intrachain disulfide bridge
XYCXGXXDVYCXFXA

SEQ ID NO: 473 - protein, 15 aa, synthetic construct
  SITE 1: note = Homoleucine
  SITE 4: note = (S)-2-amino-3,3-dimethylbutanoic acid
  SITE 6: note = (S)-2-amino-5-ureidopentanoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 14: note = 3-hydroxyvaline
  SITE 15: note = D-Alanine
  SITE 3..11: note = Intrachain disulfide bridge
XYCXGXXDVYCXFXA

SEQ ID NO: 474 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Serine
  SITE 3..11: note = Intrachain disulfide bridge
XYCXGXXDVYCXFXS

SEQ ID NO: 475 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-(methylamino)-3-(tetrahydro-2H-pyran-4-yl)pro panoic acid
  SITE 12: note = 5-fluorotryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 476 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 2-methyltryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 477 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 4-methoxyltryptophan
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXFXK

SEQ ID NO: 478 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 479 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = Amidated D-Glutamic acid
  SITE 3..11: note = Intrachain disulfide bridge
XYCXGXXDVYCXXXE

SEQ ID NO: 480 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = Amidated D-Glutamic acid
  SITE 3..11: note = Intrachain disulfide bridge
XYCXGXXDVYCXXXE

SEQ ID NO: 481 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 482 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with glutamic acid-1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 483 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 10: note = (S)-2-amino-3-(4-ureidophenyl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVXCXXXK

SEQ ID NO: 484 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 10: note = (S)-2-amino-3-(4-ureidophenyl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 4x 3-(2-aminoethoxy)propanoic acid-1,4,7,10-tetra-azacylcododecane-N,N,N,N-tetraacetic acid via the side chain
  SITE 3..11: note = Intrachain disulfide bridge
XYCXGXXDVXCXXXK

SEQ ID NO: 485 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with D-Glutamic acid-1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 486 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 487 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 10: note = (S)-2-amino-3-(4-ureidophenyl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with glutamic acid-1,4,7,10-tetra-azacylcododecane-N,N,N,N-tetraacetic acid via the side chain
  SITE 3..11: note = Intrachain disulfide bridge
XYCXGXXDVXCXXXK

SEQ ID NO: 488 - protein, 16 aa, synthetic construct
  SITE 1: note = 5-oxoproline
  SITE 2: note = Homoleucine
  SITE 5: note = (S)-2-amino-3,3-dimethylbutanoic acid
  SITE 7: note = 3-(4-piperidinyl)-Ala
  SITE 8: note = (S)-2-amino-3-(tetrahydro-2H-pyran-4-yl)propanoic acid
  SITE 11: note = (S)-2-amino-3-(4-ureidophenyl)propanoic acid
  SITE 13: note = 5-fluorotryptophan
  SITE 14: note = (2S,3S)beta-methyl-phenylalanine
  SITE 15: note = 3-hydroxyvaline
  SITE 16: note = D-Lysine modified with 1,4,7,10-tetra-azacylcododecane-N,N,N,N-tetraacetic acid
  SITE 4..12: note = Intrachain disulfide bridge
XXYCXGXXDVXCXXXK

SEQ ID NO: 489 - protein, 16 aa, synthetic construct
  SITE 1: note = 5-oxoproline
  SITE 2: note = Homoleucine
  SITE 5: note = (S)-2-amino-3,3-dimethylbutanoic acid
  SITE 7: note = 3-(4-piperidinyl)-Ala
  SITE 8: note = (S)-2-amino-3-(tetrahydro-2H-pyran-4-yl)propanoic acid
  SITE 11: note = (S)-2-amino-3-(4-ureidophenyl)propanoic acid
  SITE 13: note = 5-fluorotryptophan
  SITE 14: note = (2S,3S)beta-methyl-phenylalanine
  SITE 15: note = 3-hydroxyvaline
  SITE 16: note = D-Lysine modified with glutamic acid-1,4,7,10-tetra-azacylcododecane-N,N,N,N-tetraacetic acid via the side chain
  SITE 4..12: note = Intrachain disulfide bridge
XXYCXGXXDVXCXXXK

SEQ ID NO: 490 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 10: note = (S)-2-amino-3-(4-ureidophenyl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVXCXXXK

SEQ ID NO: 491 - protein, 16 aa, synthetic construct
  SITE 1: note = 5-oxoproline
  SITE 2: note = Homoleucine
  SITE 5: note = (S)-2-amino-3,3-dimethylbutanoic acid
  SITE 7: note = 3-(4-piperidinyl)-Ala
  SITE 8: note = (S)-2-amino-3-(tetrahydro-2H-pyran-4-yl)propanoic acid
  SITE 13: note = 5-fluorotryptophan
  SITE 14: note = (2S,3S)beta-methyl-phenylalanine
  SITE 15: note = 3-hydroxyvaline
  SITE 16: note = D-Lysine modified with 1,4,7,10-tetra-azacylcododecane-N,N,N,N-tetraacetic acid
  SITE 4..12: note = Intrachain disulfide bridge
XXYCXGXXDVYCXXXK

SEQ ID NO: 492 - protein, 16 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 10: note = (S)-2-amino-3-(4-ureidophenyl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 16: note = D-Glutamic acid
  SITE 3..11: note = Intrachain disulfide bridge
XYCXGXXDVXCXXXKE

SEQ ID NO: 493 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = Residue modified with 1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 494 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 3..11: note = Intrachain disulfide bridge
XYCXGXXDVYCXXXG

SEQ ID NO: 495 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = Beta-alanine
  SITE 3..11: note = Intrachain disulfide bridge
XYCXGXXDVYCXXXX

SEQ ID NO: 496 - protein, 15 aa, synthetic construct
  SITE 1: note = Homoleucine
  SITE 4: note = (S)-2-amino-3,3-dimethylbutanoic acid
  SITE 6: note = Acetylated Lysine
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGKXDVYCXXXA

SEQ ID NO: 497 - protein, 15 aa, synthetic construct
  SITE 1: note = Homoleucine
  SITE 4: note = (S)-2-amino-3,3-dimethylbutanoic acid
  SITE 6: note = 3-(1-acetylpiperidin-4-yl)alanine
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 498 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(2-amino-2-oxoethyl)piperidin-4-yl )propanoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 499 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (3-(4-piperidinyl)-alanine)-(1-amino-3,6,9,12-tetra oxapentadecan-15-oic acid modified with acetyl)
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 500 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = (S)-2-amino-3-(naphthalen-1-yl)propanoic acid
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 501 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = Epsilon-Lysine modified with Val-Met-4-(aminomethyl)benzoic acid-1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 502 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = Residue modified with epsilon-Lysine-Val-Met-4-(aminomethyl)benzoic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Serine
  SITE 3..11: note = Intrachain disulfide bridge
XYCXGXEDVYCXXXS

SEQ ID NO: 503 - protein, 15 aa, synthetic construct
  SITE 1: note = Homoleucine
  SITE 4: note = (S)-2-amino-3,3-dimethylbutanoic acid
  SITE 6: note = Residue modified with epsilon-Lysine-Val-Met-4-(aminomethyl)benzoic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Serine
  SITE 3..11: note = Intrachain disulfide bridge
XYCXGEXDVYCXXXS

SEQ ID NO: 504 - protein, 15 aa, synthetic construct
  SITE 1: note = Homoleucine
  SITE 4: note = (S)-2-amino-3,3-dimethylbutanoic acid
  SITE 6: note = 3-(1-methylpiperidin-4-yl)alanine
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 505 - protein, 15 aa, synthetic construct
  SITE 1: note = Homoleucine
  SITE 4: note = (S)-2-amino-3,3-dimethylbutanoic acid
  SITE 6: note = N6,N6-dimethyl-L-lysine
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 506 - protein, 15 aa, synthetic construct
  SITE 1: note = Homoleucine
  SITE 4: note = (S)-2-amino-3,3-dimethylbutanoic acid
  SITE 6: note = (S)-5-amino-5-carboxy-N,N,N-trimethylpentan-1-amini um
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 507 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = Residue modified with 4x 3-(2-aminoethoxy)propanoic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXKDVYCXXXA

SEQ ID NO: 508 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 8: note = (R)-2-amino-3-sulfopropanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXXVYCXXXK

SEQ ID NO: 509 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with homoglutamic acid-1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 510 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 511 - protein, 15 aa, synthetic construct
  SITE 1: note = Homoleucine
  SITE 4: note = Beta-methyl isoleucine
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 512 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with homoglutamic acid-1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 513 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 15: note = D-Lysine modified with homoglutamic acid-1,4,7,10-tetra-azacylcododecane-N,N,N,N-tetraacetic acid via the side chain
  SITE 3..11: note = Intrachain disulfide bridge
XYCXGXXDVYCXXPK

SEQ ID NO: 514 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with homoglutamic acid-homoglutamic acid-1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 515 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-(methylamino)-3-(tetrahydro-2H-pyran-4-yl)pro panoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 516 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(1-(carboxymethyl)piperidin-4-yl)prop anoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 517 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(1-(carboxymethyl)piperidin-4-yl)prop anoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 518 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-4-(4-(2-amino-2-carboxyethyl)piperidin-1-yl)-4- oxobutanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 519 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-succinic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 520 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 521 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 522 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXXK

SEQ ID NO: 523 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 15: note = D-Lysine modified with 1,4,7,10-tetra-azacylcododecane-N,N,N,N-tetraacetic acid
  SITE 3..11: note = Intrachain disulfide bridge
XYCXGXXDVYCXXPK

SEQ ID NO: 524 - protein, 15 aa, synthetic construct
  SITE 1: note = Homoleucine
  SITE 4: note = (S)-2-amino-3,3-dimethylbutanoic acid
  SITE 6: note = (S)-2-amino-3-(tetrahydro-2H-pyran-4-yl)propanoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 525 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 10: note = (S)-2-amino-3-(4-ureidophenyl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVXCXXXA

SEQ ID NO: 526 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  SITE 10: note = (S)-2-amino-3-(4-ureidophenyl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVXCXXXA

SEQ ID NO: 527 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-Ala
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with homoglutamic acid-homoglutamic acid-homoglutamic acid-1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 528 - protein, 15 aa, synthetic construct
  SITE 1: note = Homoleucine
  SITE 4: note = (S)-2-amino-3,3-dimethylbutanoic acid
  SITE 6: note = (S)-4-(4-(2-amino-2-carboxyethyl)piperidin-1-yl)-4- oxobutanoic acid
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with homoglutamic acid-1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 529 - protein, 16 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with homoglutamic acid-1,4,7,10-tetra-azacylcododecane-N,N,N,N-tetraacetic acid via the side chain
  SITE 16: note = D-Glutamic acid
  SITE 3..11: note = Intrachain disulfide bridge
XYCXGXXDVYCXXXKE

SEQ ID NO: 530 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with homoglutamic acid-tranexamic acid-1,4,7,10-tetra-azacylcododecane-N,N,N,N-tetraacetic acid via the side chain
  SITE 3..11: note = Intrachain disulfide bridge
XYCXGXXDVYCXXXK

SEQ ID NO: 531 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 10: note = (S)-2-amino-3-(4-ureidophenyl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with homoglutamic acid-1,4,7,10-tetra-azacylcododecane-N,N,N,N-tetraacetic acid via the side chain
  SITE 3..11: note = Intrachain disulfide bridge
XYCXGXXDVXCXXXK

SEQ ID NO: 532 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = Residue modified with 1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXKDVYCXXXA

SEQ ID NO: 533 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = 3-(4-piperidinyl)-alanine modified with 1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 534 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = 3-(4-piperidinyl)-alanine modified with glutamic acid-1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 535 - protein, 16 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = (S)-2-amino-3-(tetrahydro-2H-pyran-4-yl)propanoic acid
  SITE 10: note = (S)-2-amino-3-(4-ureidophenyl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Lysine modified with 1,4,7,10-tetra-azacylcododecane-N,N,N,N-tetraacetic acid
  SITE 16: note = D-Glutamic acid
  SITE 3..11: note = Intrachain disulfide bridge
XYCXGXXDVXCXXXKE

SEQ ID NO: 536 - protein, 15 aa, synthetic construct
  SITE 1: note = Norleucine
  SITE 8: note = (S)-2,4-diaminobutanoic acid
  SITE 15: note = Amidated D-Lysine modified with 1,4,7,10-tetra-azacylcododecane-N,N,N,N-tetraacetic acid via the side chain
  SITE 3..11: note = Intrachain disulfide bridge
XYCVGKYXVYCWFTK

SEQ ID NO: 537-552 - skipped (16 entries)

SEQ ID NO: 553 - protein, 534 aa, synthetic construct
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYDKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 554 - protein, 15 aa, synthetic construct
  VARIANT 1: note = (S) or (R)-2-amino-4-cyclohexylbutanoic acid
  VARIANT 2: note = D or L Tyrosine
  VARIANT 3: note = D or L Cysteine
  VARIANT 4: note = (S) or (R)-2-amino-3,3-dimethylbutanoic acid
  VARIANT 5: note = D or L Glycine
  VARIANT 6: note = D or L 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  VARIANT 7: note = (S) or (R)-2-amino-3-(tetrahydro-2H-pyran-4-yl)propanoic acid
  VARIANT 8: note = D or L Aspartic Acid
  VARIANT 9: note = D or L Valine
  VARIANT 10: note = D or L Tyrosine
  VARIANT 11: note = D or L Cysteine
  VARIANT 12: note = D or L 5-fluorotryptophan
  VARIANT 13: note = D or L Phenylalanine
  VARIANT 14: note = D or L 3-hydroxyvaline
  VARIANT 15: note = D or L Alanine
  SITE 3..11: note = Intrachain disulfide bridge
XYCXGXXDVYCXFXA

SEQ ID NO: 555 - protein, 15 aa, synthetic construct
  VARIANT 1: note = D or L Homoleucine
  VARIANT 2: note = D or L Tyrosine
  VARIANT 3: note = D or L Cysteine
  VARIANT 4: note = (S) or (R)-2-amino-3,3-dimethylbutanoic acid
  VARIANT 5: note = D or L Glycine
  VARIANT 6: note = D or L 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  VARIANT 7: note = (S) or (R)-2-amino-3-(tetrahydro-2H-pyran-4-yl)propanoic acid
  VARIANT 8: note = D or L Aspartic Acid
  VARIANT 9: note = D or L Valine
  VARIANT 10: note = D or L Tyrosine
  VARIANT 11: note = D or L Cysteine
  VARIANT 12: note = D or L 5-fluorotryptophan
  VARIANT 13: note = (2S,3S) or (2R,3R)beta-methyl-phenylalanine
  VARIANT 14: note = D or L 3-hydroxyvaline
  VARIANT 15: note = D or L Alanine
  SITE 3..11: note = Intrachain disulfide bridge
XYCXGXXDVYCXXXA

SEQ ID NO: 556 - protein, 15 aa, synthetic construct
  VARIANT 1: note = D or L Homoleucine
  VARIANT 2: note = D or L Tyrosine
  VARIANT 3: note = D or L Cysteine
  VARIANT 4: note = (S) or (R)-2-amino-3,3-dimethylbutanoic acid
  VARIANT 5: note = D or L Glycine
  VARIANT 6: note = (S) or (R)-2-amino-3-(1-(carboxymethyl)piperidin-4-yl)propanoic acid
  VARIANT 7: note = D or L 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  VARIANT 8: note = D or L Aspartic Acid
  VARIANT 9: note = D or L Valine
  VARIANT 10: note = D or L Tyrosine
  VARIANT 11: note = D or L Cysteine
  VARIANT 12: note = D or L 5-fluorotryptophan
  VARIANT 13: note = (2S,3S) or (2R,3R)beta-methyl-phenylalanine
  VARIANT 14: note = D or L 3-hydroxyvaline
  VARIANT 15: note = Amidated D or L Glutamic acid
  SITE 3..11: note = Intrachain disulfide bridge
XYCXGXXDVYCXXXE

SEQ ID NO: 557 - protein, 15 aa, synthetic construct
  VARIANT 1: note = D or L Homoleucine
  VARIANT 2: note = D or L Tyrosine
  VARIANT 3: note = D or L Cysteine
  VARIANT 4: note = (S) or (R)-2-amino-3,3-dimethylbutanoic acid
  VARIANT 5: note = D or L Glycine
  VARIANT 6: note = (S) or (R)-2-amino-3-(1-(carboxymethyl)piperidin-4-yl)propanoic acid
  VARIANT 7: note = D or L 3-(4-piperidinyl)-alanine modified with 1-amino-3,6,9,12-tetraoxapentadecan-15-oic acid-1,4,7,10-tetra-azacylcododecane-N,N,N,N-tetraacetic acid via the side chain
  VARIANT 8: note = D or L Aspartic Acid
  VARIANT 9: note = D or L Valine
  VARIANT 10: note = D or L Tyrosine
  VARIANT 11: note = D or L Cysteine
  VARIANT 12: note = D or L 5-fluorotryptophan
  VARIANT 13: note = (2S,3S) or (2R,3R)beta-methyl-phenylalanine
  VARIANT 14: note = D or L 3-hydroxyvaline
  VARIANT 15: note = D or L Alanine
  SITE 3..11: note = Intrachain disulfide bridge
XYCXGXXDVYCXXXA

SEQ ID NO: 558 - protein, 9 aa, synthetic construct
  VARIANT 2..9: note = This region may encompass 1-8 residues
KGGGGGGGG

SEQ ID NO: 559 - protein, 9 aa, synthetic construct
  VARIANT 2..9: note = This region may encompass 1-8 residues
YGGGGGGGG

SEQ ID NO: 560 - protein, 15 aa, synthetic construct
  SITE 3..11: note = Intrachain disulfide bridge
LYCVGKADVYCWFVE

SEQ ID NO: 561 - protein, 15 aa, synthetic construct
  SITE 1: note = (S)-2-amino-4-cyclohexylbutanoic acid
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-[177Lu]Lu-labeled 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Alanine
  SITE 3..11: note = Intrachain disulfide bridge
XYCXGXXDVYCXFXA

SEQ ID NO: 562 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-[177Lu]Lu-labeled 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 563 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = 3-(4-piperidinyl)-alanine modified with 1-amino-3,6,9,12-tetraoxapentadecan-15-oic acid-[177Lu]Lu-labeled 1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = Amidated D-Glutamic acid
  SITE 3..11: note = Intrachain disulfide bridge
XYCXGXXDVYCXXXE

SEQ ID NO: 564 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = 3-(4-piperidinyl)-alanine modified with 1-amino-3,6,9,12-tetraoxapentadecan-15-oic acid-[177Lu]Lu-labeled 1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 565 - protein, 15 aa, synthetic construct
  SITE 1: note = (S)-2-amino-4-cyclohexylbutanoic acid
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-[225Ac]Ac-labeled 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 14: note = 3-hydroxyvaline
  SITE 15: note = D-Alanine
  SITE 3..11: note = Intrachain disulfide bridge
XYCXGXXDVYCXFXA

SEQ ID NO: 566 - protein, 15 aa, synthetic construct
  SITE 1: note = Homoleucine
  SITE 4: note = (S)-2-amino-3,3-dimethylbutanoic acid
  SITE 6: note = 3-(4-piperidinyl)-alanine modified with 1-amino-3,6,9,12-tetraoxapentadecan-15-oic acid-[225Ac]Ac-labeled 1,4,7,10-tetra-azacylcododecane-N,N,N,N-tetraacetic acid via the side chain
  SITE 7: note = (S)-2-amino-3-(tetrahydro-2H-pyran-4-yl)propanoic acid
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 567 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = 3-(4-piperidinyl)-alanine modified with 1-amino-3,6,9,12-tetraoxapentadecan-15-oic acid-[225Ac]Ac-labeled 1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = Amidated D-Glutamic acid
  SITE 3..11: note = Intrachain disulfide bridge
XYCXGXXDVYCXXXE

SEQ ID NO: 568 - protein, 15 aa, synthetic construct
  SITE 1: note = Homoleucine
  SITE 4: note = (S)-2-amino-3,3-dimethylbutanoic acid
  SITE 6: note = (S)-2-amino-3-(1-(carboxymethyl)piperidin-4-yl)prop anoic acid
  SITE 7: note = 3-(4-piperidinyl)-alanine modified with 1-amino-3,6,9,12-tetraoxapentadecan-15-oic acid-[225Ac]Ac-labeled 1,4,7,10-tetra-azacylcododecane-N,N,N,N-tetraacetic acid via the side chain
  SITE 12: note = 5-fluorotryptophan
  SITE 13: note = (2S,3S)beta-methyl-phenylalanine
  SITE 14: note = 3-hydroxyvaline
  SITE 15: note = D-Alanine
  SITE 3..11: note = Intrachain disulfide bridge
XYCXGXXDVYCXXXA

SEQ ID NO: 569 - protein, 13 aa, synthetic construct
  SITE 1: note = Norleucine
  SITE 13: note = Amidated residue
XYCVGKYDVYCWF

SEQ ID NO: 570 - protein, 15 aa, synthetic construct
  SITE 1: note = Norleucine
  SITE 15: note = Amidated D-Lysine modified with 1,4,7,10-tetra-azacylcododecane-N,N,N,N-tetraacetic acid via the side chain
XYCVGKYDVYCWFTK

SEQ ID NO: 571 - protein, 15 aa, synthetic construct
  SITE 1: note = Norleucine
  SITE 15: note = Amidated D-Lysine modified with 1,4,7,10-tetra-azacylcododecane-N,N,N,N-tetraacetic acid via the side chain
XYCVGKYDVYCWFTK

SEQ ID NO: 572 - protein, 15 aa, synthetic construct
  SITE 1: note = Homoleucine
  SITE 3: note = Propargyl alanine
  SITE 6: note = N6-(1-(4,4-dimethyl-2,6-dioxocyclohexylidene)ethyl) -D-Lysine
  SITE 11: note = (S)-2-amino-3-azidopropanoic acid
  SITE 12: note = 5-fluorotryptophan
  SITE 15: note = Amidated D-Lysine
XYXVGKYDVYXXFTK

SEQ ID NO: 573 - protein, 15 aa, synthetic construct
  SITE 1: note = Homoleucine
  SITE 3: note = Propargyl alanine
  SITE 6: note = N6-(1-(4,4-dimethyl-2,6-dioxocyclohexylidene)ethyl) -D-Lysine
  SITE 11: note = (S)-2-amino-3-azidopropanoic acid
  SITE 12: note = 5-fluorotryptophan
  SITE 15: note = Amidated D-Lysine
  SITE 3..11: note = Intrachain triazole bridge
XYXVGKYDVYXXFTK

SEQ ID NO: 574 - protein, 15 aa, synthetic construct
  SITE 1: note = Homoleucine
  SITE 3: note = Propargyl alanine
  SITE 6: note = N6-(1-(4,4-dimethyl-2,6-dioxocyclohexylidene)ethyl) -D-Lysine
  SITE 11: note = (S)-2-amino-3-azidopropanoic acid
  SITE 12: note = 5-fluorotryptophan
  SITE 15: note = Amidated D-Lysine modified with 1,4,7,10-tetra-azacylcododecane-N,N,N,N-tetraacetic acid via the side chain
  SITE 3..11: note = Intrachain triazole bridge
XYXVGKYDVYXXFTK